(19) United States
(12) Patent Application Publication
(10) Pub. No.: US 2023/0150972 A1
(43) Pub. Date: May 18, 2023

(54) PYRIDINYL DERIVATIVES AS SODIUM CHANNEL ACTIVATORS

(71) Applicant: Xenon Pharmaceuticals Inc., Burnaby (CA)

(72) Inventors: Verner Alexander Lofstrand, Burnaby (CA); Jung Yun Kim, Burnaby (CA); Helen Clement, Burnaby (CA); Kristen Nicole Burford, Burnaby (CA); Paul Charifson, Burnaby (CA); Michael Clark, Burnaby (CA)

(21) Appl. No.: 17/951,549

(22) Filed: Sep. 23, 2022

Related U.S. Application Data
(60) Provisional application No. 63/248,341, filed on Sep. 24, 2021.

(51) Int. Cl. C07D 401/12; C07D 405/14; C07D 401/14; C07D 413/14; C07D 413/12; C07D 403/12; C07D 211/56; C07D 405/12; C07D 498/08; C07D 487/08; C07D 471/08; C07D 487/10; C07D 407/14; C07D 417/12; C07D 498/04; C07D 401/04; C07D 497/08; C07D 239/42; C07D 409/14

(52) U.S. Cl. CPC C07D 401/12; C07D 405/14; C07D 401/14; C07D 413/14; C07D 413/12; C07D 403/12; C07D 211/56; C07D 405/12; C07D 498/08; C07D 487/08; C07D 471/08; C07D 487/10; C07D 407/14; C07D 417/12; C07D 498/04; C07D 401/04; C07D 497/08; C07D 239/42; C07D 409/14

(57) ABSTRACT

The present disclosure is directed to compounds of formula (I):

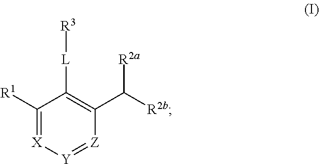

wherein X, Y, Z, R¹, R^{2a}, R^{2b}, R³, and L are as described herein, as stereoisomers, enantiomers, or tautomers thereof or mixtures thereof; or pharmaceutically acceptable salts, solvates, or prodrugs thereof, and pharmaceutical compositions comprising the compounds of formula (I), as described herein, which are useful as voltage-gated sodium channel modulators and are therefore are useful in treating seizure disorders such as epilepsy.

### TECHNICAL FIELD

This disclosure is directed to pyridinyl derivatives, as stereoisomers, enantiomers, or tautomers thereof or mixtures thereof; or pharmaceutically acceptable salts, solvates, or prodrugs thereof, and pharmaceutical compositions comprising the pyridinyl derivatives, which are useful as voltage-gated sodium channel activators and are therefore are useful in treating seizure disorders such as epilepsy.

### BACKGROUND

Epilepsy is a common seizure disorder, with a worldwide estimated prevalence of 0.7% of the population (50 million people) (see Hirtz, D. et al., Neurology. (2007), 68:326-337). It is characterized by abnormal electrical activities in the brain leading to seizures. For epidemiological purposes, the definition requires more than one unprovoked seizure of any type.

Patients with epilepsy have an increased mortality risk compared with the general population due primarily to the etiology of the disease. However, in patients with uncontrolled epilepsy, the greatest seizure-related risk of mortality is due to sudden unexpected death in epilepsy (SUDEP) (see, Hitiris, N. et al., Epilepsy and Behavior (2007), 10:363-376. Patients who participate in clinical trials of investigational antiepileptic drugs (AEDs) generally have had epilepsy for more than 10 years and have failed multiple AED therapies.

The pathophysiology of most forms of epilepsy remains poorly understood, but it is known that epileptic seizures arise from an excessively synchronous and sustained firing of a group of neurons. Persistent increase in neuronal excitability is common to all epileptic syndromes. The therapeutic strategy in treating epilepsy involves reducing neuronal excitability through various mechanistic pathways. Over the past two decades, several new AEDs were developed and marketed to expand the therapeutic spectrum by targeting different mechanisms of action and to improve the risk/benefit profile. Currently available AEDs are considered to act by inhibition of synaptic vesicle glycoprotein, potentiation of the inhibitory GABAergic neurotransmission, reduction of glutamate-mediated excitatory neurotransmission, or inhibition of voltage-gated sodium or calcium channels. Despite this, up to 30% of patients remain refractory to conventional treatment and continue to have uncontrolled seizures (see Brown, D. A. et al., Nature (1980), 283:673-676, and Elger, C. E. et al., Epilepsy Behav. (2008), 12:501-539. The quality of life in refractory patients is poor; they cannot drive a car, and they have difficulty working or living independently. Additionally, many patients have behavioral, neurological, and/or intellectual disturbances as sequelae of their seizure disorder. Current agents have minimal to no effects on neuronal sodium-gated channels, in spite of the fact that these channels have a major role in the control of neuronal excitability. Medicines with novel mechanisms of action, or medicines that improve on the already marketed AEDs are therefore needed to address the significant unmet clinical need for seizure control in patients with treatment-resistant epilepsy.

Naᵥ1.1 is a voltage-gated sodium channel (Naᵥ), comprising one pore-forming α-subunit encoded by SCN1A and two associated β-subunits encoded by SCN1B-SCN4B. Naᵥ1.1 as well as its subfamilies (Naᵥ1.2, Naᵥ1.3 and Naᵥ1.6), is predominantly expressed in the central nervous system (CNS) (Catterall, W. A., J Physiol (2012), Vol. 590, pp. 2577-2589, and Catterall, W. A., Neurochem Res (2017), Vol. 42, pp. 2495-2504). Naᵥ1.1 is largely expressed in parvalbuminpositive fast spiking interneurons (FSINs) and is involved in membrane depolarization and action potential (AP) firing (Ogiwara, I. et al., J Neurosci (2007), Vol. 27, pp. 5903-5914). Therefore, loss of function of the Naᵥ1.1 channels could lead to disinhibition of excitatory pyramidal neurons causing various diseases of the CNS (Han, S. et al., Nature (2012), Vol. 489, pp. 385-390, Oakley, J. C. et al. Epilepsia (2011), Vol. 52 (Suppl. 2), pp. 59-61, and Verret, L. et al., Cell (2012), Vol. 149, pp. 708-721). Dravet syndrome is a rare genetic epileptic encephalopathy, where more than 70% of patients have de novo heterozygous mutations of the SCN1A gene (Catterall, W. A., Ann Rev Pharmacol Toxicol (2014), Vol. 54, pp. 317-338). In these mutations, a loss of function of the Naᵥ1.1 channels has been reported (Mantegazza, M. et al., Proc Natl Acad Sci USA (2005), Vol. 102, pp. 18177-18182). The genetic link between Dravet syndrome patients and Naᵥ1.1 channels suggest that a brain penetrant Naᵥ1.1 activator could possess significant therapeutic potential for treating Dravet syndrome (Jensen, H. S. et al., Trends Pharmacol Sci (2014), Vol. 35, pp. 113-118, and Richards, K. L. et al., Proc Natl Acad Sci USA (2018), Vol. 115, pp. E8077-E8085). However, potent and selective Naᵥ1.1 activators have not been reported to date. Recently, a few Naᵥ1.1 activators have been reported by Lundbeck: a 2-methylbenzamide derivative (Crestey, F. et al., ACS Chem Neurosci (2015), Vol. 6, pp. 1302-1308), AA43279 (Frederiksen, K. et al., Eur J Neurosci (2017), Vol. 46, pp. 1887-1896) and Lu AE98134 (von Schoubyea, N. L. et al., Neurosci Lett (2018), Vol. 662, pp. 29-35). The most recently developed activator, Lu AE98134, increases the total area under the curve for the duration of the depolarizing pulse from 1 μM in Naᵥ1.1-expressing HEK cells, while issues of low selectivity against Naᵥ1.5 and moderate selectivity against Naᵥ1.2 were observed. Biologically, Naᵥ1.5 is a major cardiac sodium channel (Vincent, G. M., Annu Rev Med (1998), Vol. 49, pp. 263-274) and Naᵥ1.2 is dominantly expressed in excitatory neurons (Gong, B. et al., J Comp Neurol (1999), Vol. 412, pp. 342-352, and Hu, W. et al., Nat Neurosci (2009), Vol. 12, pp. 996-1002). Therefore, high selectivity against Naᵥ1.5 and Naᵥ1.2 is preferable for drug candidates. On the other hand, the electrophysiology data regarding Lu AE98134 reveals promising potency as a Naᵥ1.1 activator for increasing the excitability of FSINs. The discovery of a 4-phenyl-2-(pyrrolidinyl)nicotinamide derivative as a highly potent Naᵥ1.1 activator with improved selectivity against Naᵥ1.2 and Naᵥ1.5 compared with previously reported Naᵥ1.1 activators was recently published (Miyazaki, T. et al., Bioorg Med Chem Lett (2019), Vo. 29, No. 6, pp. 815-820).

While significant advances have been made in this field, there remains a substantial need for compounds which are voltage-gated sodium channel activators, thereby being useful in treating seizure disorders, preferably epilepsy, in a mammal, preferably a human.

### BRIEF SUMMARY

The present disclosure is directed to pyridinyl derivatives, as stereoisomers, enantiomers, or tautomers thereof or mixtures thereof; or pharmaceutically acceptable salts, solvates, or prodrugs thereof, and pharmaceutical compositions comprising the pyridinyl derivatives, which are useful as voltage-gated sodium channel activators, particularly Naᵥ1.1 activators, and are therefore are useful in treating seizure disorders such as epilepsy and Dravet syndrome.

Accordingly, some embodiments of the present disclosure provide compounds of formula (I):

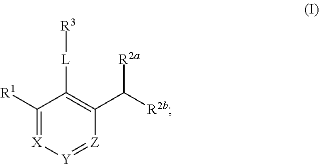

wherein:
X, Y, and Z are each independently N or CR^{1b}, provided that at least one and no more than two of X, Y, and Z are N;
L is a direct bond, —NR⁴C(═O)—, or —C(═O)NR⁴—;
R¹ is methoxy, —R⁵N(R⁶)₂, alkenyl, or has one of the following structures:

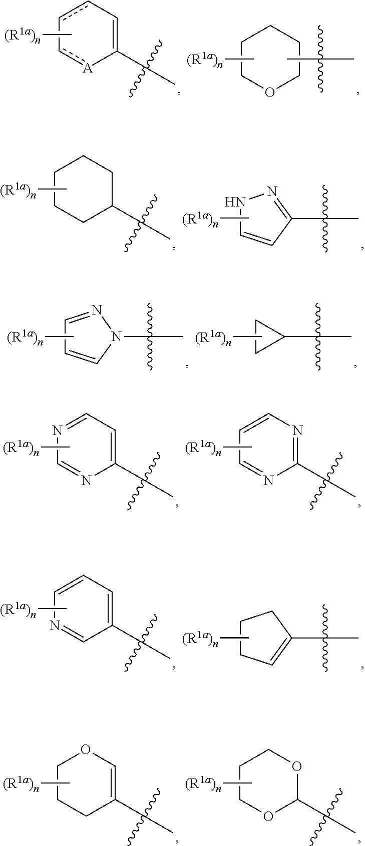

wherein:
each [symbol] is independently a single or double bond such that all valences are satisfied;
   each R^{1a} is independently alkyl, halo, haloalkyl, —R⁵OR⁶, —R⁵N(R⁶)₂, —R⁵OC(═O)R⁶, optionally substituted cycloalkyl, or —R⁵C(═O)OR⁶;
A is O, N, or C;
each R^{1b} is independently hydrogen, halo, alkyl, or haloalkyl;
R^{2a} and R^{2b}, together with the carbon to which they are attached, form an optionally substituted cycloalkyl, an optionally substituted cycloalkenyl, an optionally substituted aryl, an optionally substituted heterocyclyl, or an optionally substituted heteroaryl;
   or R^{2a} is hydrogen or alkyl and R^{2b} is an optionally substituted heterocyclyl or an optionally substituted cycloalkyl;
   or R^{2a} and R^{2b} are both alkyl;
   or R^{2a} is alkyl and R^{2b} is haloalkoxy;
R³ is alkyl, cyanoalkyl, —R⁵OR⁶, —R⁵N(R⁶)₂, an optionally substituted cycloalkyl, an optionally substituted aryl, an optionally substituted heterocyclyl, an optionally substituted heteroaryl, an optionally substituted cycloalkylalkyl, an optionally substituted heterocyclylalkyl;
R⁴ is hydrogen or alkyl;
each R⁵ is independently a direct bond or an optionally substituted alkylene chain;
each R⁶ is independently hydrogen, alkyl, haloalkyl, optionally substituted cycloalkyl, or optionally substituted arylalkyl;
or two R⁶'s, together with the nitrogen to which they are both attached, form an optionally substituted heterocyclyl; and
n is 0, 1, 2, 3, 4, or 5,
   as a stereoisomer, enantiomer, or tautomer thereof or a mixture thereof; or a pharmaceutically acceptable salt, solvate, isotopologue, or prodrug thereof.

In some embodiments, the present disclosure is directed to compounds of formula (II):

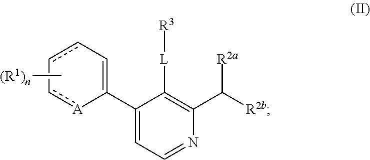

wherein:
each [symbol] is independently a single or double bond;
A is O, N, or C;
L is a direct bond, —NR⁴C(═O)—, or —C(═O)NR⁴—;
each R¹ is independently alkyl, halo, haloalkyl, —R⁵OR⁶, —R⁵N(R⁶)₂, —R⁵OC(═O)R⁶, or —R⁵C(═O)OR⁶;
R^{2a} and R^{2b}, together with the carbon to which they are attached, form an optionally substituted cycloalkyl, an optionally substituted cycloalkenyl, an optionally substituted aryl, an optionally substituted heterocyclyl, or an optionally substituted heteroaryl;
R³ is an optionally substituted cycloalkyl, an optionally substituted aryl, an optionally substituted heterocyclyl, or an optionally substituted heteroaryl;
R⁴ is hydrogen or alkyl;
each R⁵ is independently a direct bond or an optionally substituted alkylene chain;
each R⁶ is independently hydrogen, alkyl, haloalkyl, or optionally substituted cycloalkyl;
or two R⁶'s, together with the nitrogen to which they are both attached, form an optionally substituted heterocyclyl; and
n is 0, 1, 2, 3, 4, or 5,
   as a stereoisomer, enantiomer, or tautomer thereof or a mixture thereof; or a pharmaceutically acceptable salt, solvate, or prodrug thereof.

In other embodiments, this disclosure is directed to pharmaceutical compositions comprising a pharmaceutically acceptable excipient and a compound of formula (I) or (II), as a stereoisomer, enantiomer, or tautomer thereof or a mixture thereof; or a pharmaceutically acceptable salt, solvate, or prodrug thereof, as described above.

In other embodiments, this disclosure is directed to methods of treating a disease or condition in a mammal modulated by a voltage-gated sodium channel, wherein the methods comprise administering to a mammal in need thereof a therapeutically effective amount of a compound of formula (I) or (II), as a stereoisomer, enantiomer, or tautomer thereof or a mixture thereof; or a pharmaceutically acceptable salt, solvate, or prodrug thereof, as described above.

In other embodiments, this disclosure is directed to methods for the treatment of epilepsy and/or epileptic seizure disorder in a mammal, preferably a human, wherein the methods comprise administering to the mammal in need thereof a therapeutically effective amount of a compound of formula (I) or (II), as set forth above, as a stereoisomer, enantiomer, or tautomer thereof or mixtures thereof; or a pharmaceutically acceptable salt, solvate, or prodrug thereof, or a pharmaceutical composition comprising a therapeutically effective amount of a compound of formula (I) or (II), as set forth above, as a stereoisomer, enantiomer, or tautomer thereof or mixtures thereof, or a pharmaceutically acceptable salt, solvate, or prodrug thereof, and a pharmaceutically acceptable excipient.

In other embodiments, this disclosure is directed to methods of preparing a compound of formula (I) or (II), as set forth above, as a stereoisomer, enantiomer, or tautomer thereof or mixtures thereof; or a pharmaceutically acceptable salt, solvate, or prodrug thereof, or a pharmaceutical composition comprising a therapeutically effective amount of a compound of formula (I) or (II), as set forth above, as a stereoisomer, enantiomer, or tautomer thereof or mixtures thereof, or a pharmaceutically acceptable salt, solvate, or prodrug thereof, and a pharmaceutically acceptable excipient.

In other embodiments, this disclosure is directed to pharmaceutical therapy in combination with one or more other compounds of formula (I) or (II) or one or more other accepted therapies or as any combination thereof to increase the potency of an existing or future drug therapy or to decrease the adverse events associated with the accepted therapy. In one embodiment, this disclosure is directed to a pharmaceutical composition combining a compound of formula (I) or (II) with established or future therapies for the indications listed herein.

### DETAILED DESCRIPTION

### Definitions

Certain chemical groups named herein may be preceded by a shorthand notation indicating the total number of carbon atoms that are to be found in the indicated chemical group. For example; C₇-C₁₂alkyl describes an alkyl group, as defined below, having a total of 7 to 12 carbon atoms, and C₄-C₁₂cycloalkylalkyl describes a cycloalkylalkyl group, as defined below, having a total of 4 to 12 carbon atoms. The total number of carbons in the shorthand notation does not include carbons that may exist in substituents of the group described.

In addition to the foregoing, as used in the specification and appended claims, unless specified to the contrary, the following terms have the meaning indicated:

“Compound of the disclosure” or “compounds of the disclosure” refer to compounds of formula (I) or (II), as described above in the Brief Summary, as stereoisomers, enantiomers, or tautomers thereof or mixtures thereof; or pharmaceutically acceptable salts, solvates, or prodrugs thereof.

“Amino” refers to the —NH₂ radical.

“Cyano” refers to the —CN radical.

“Hydroxy” refers to the —OH radical.

“Imino” refers to the ═NH substituent.

“Nitro” refers to the —NO₂ radical.

“Oxo” refers to the ═O substituent.

“Thioxo” refers to the ═S substituent.

“Trifluoromethyl” refers to the —CF₃ radical.

“Alkyl” refers to a straight or branched hydrocarbon chain radical consisting solely of carbon and hydrogen atoms, containing no unsaturation, having from one to twelve carbon atoms, preferably one to eight carbon atoms or one to six carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, an alkyl group may be optionally substituted by one of the following groups: alkyl, alkenyl, halo, haloalkenyl, cyano, nitro, aryl, cycloalkyl, heterocyclyl, heteroaryl, oxo,

trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²², —N(R²⁰)C(O)R²², —N(R²⁰)S(O)ₜR²² (where t is 1 to 2), —S(O)ₜOR²² (where t is 1 to 2), —S(O)ₚR²² (where p is 0 to 2), and —S(O)ₜN(R²⁰)₂ (where t is 1 to 2) where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R²² is alkyl, haloalkyl, cycloalkyl, cycloalkylalkyl, aryl, aralkyl, heterocyclyl, heterocyclylalkyl, heteroaryl or heteroarylalkyl.

“Alkoxy” refers to a radical having the following formula —OR^{a} where R^{a} is an alkyl radical as defined above.

“Alkenyl” refers to a straight or branched hydrocarbon chain radical group consisting solely of carbon and hydrogen atoms, containing at least one double bond, having from two to twelve carbon atoms, preferably two to eight carbon atoms and which is attached to the rest of the molecule by a single bond, e.g., ethenyl, prop-1-enyl, but-1-enyl, pent-1-enyl, penta-1,4-dienyl, and the like. Unless stated otherwise specifically in the specification, an alkenyl group may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²², —N(R²⁰)C(O)R²², —N(R²⁰)S(O)ₜR²² (where t is 1 to 2), —S(O)ₜOR²² (where t is 1 to 2), —S(O)ₚR²² (where p is 0 to 2), and —S(O)ₜN(R²⁰)₂ (where t is 1 to 2) where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R²² is alkyl, haloalkyl, cycloalkyl, cycloalkylalkyl, aryl, aralkyl, heterocyclyl, heterocyclylalkyl, heteroaryl or heteroarylalkyl.

“Alkynyl” refers to a straight or branched hydrocarbon chain radical group consisting solely of carbon and hydrogen atoms, containing at least one triple bond, having from two to twelve carbon atoms, preferably one to eight carbon atoms and which is attached to the rest of the molecule by a single bond, e.g., ethynyl, propynyl, butynyl, pentynyl, hexynyl, and the like. Unless stated otherwise specifically in the specification, an alkynyl group is optionally substituted by one or more of the following groups: alkyl, alkenyl, halo, haloalkenyl, cyano, nitro, aryl, cycloalkyl, heterocyclyl, heteroaryl, oxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²², —N(R²⁰)C(O)R²², —N(R²⁰)S(O)ₜR²² (where t is 1 to 2), —S(O)ₜOR²² (where t is 1 to 2), —S(O)ₚR²² (where p is 0 to 2), or —S(O)ₜN(R²⁰)₂ (where t is 1 to 2), where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R²² is alkyl, haloalkyl, cycloalkyl, cycloalkylalkyl, aryl, aralkyl, heterocyclyl, heterocyclylalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²², —N(R²⁰)C(O)R²², —N(R²⁰)S(O)ₜR²² (where t is 1 to 2), —S(O)ₜOR²² (where t is 1 to 2), —S(O)ₚR²² (where p is 0 to 2), and —S(O)ₜN(R²⁰)₂ (where t is 1 to 2) where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R²² is alkyl, haloalkyl, cycloalkyl, cycloalkylalkyl, aryl, aralkyl, heterocyclyl, heterocyclylalkyl, heteroaryl or heteroarylalkyl.

“Alkenylene” or “alkenylene chain” refers to a straight or branched divalent hydrocarbon chain linking the rest of the molecule to a radical group, consisting solely of carbon and hydrogen, containing at least one double bond and having from two to twelve carbon atoms, e.g., ethenylene, propenylene, n-butenylene, and the like. The alkenylene chain is attached to the rest of the molecule through a single bond and to the radical group through a double bond or a single bond. The points of attachment of the alkenylene chain to the rest of the molecule and to the radical group can be through one carbon or any two carbons within the chain. Unless stated otherwise specifically in the specification, an alkenylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²², —N(R²⁰)C(O)R²², —N(R²⁰)S(O)ₜR²² (where t is 1 to 2), —S(O)ₜOR²² (where t is 1 to 2), —S(O)ₚR²² (where p is 0 to 2), and —S(O)ₜN(R²⁰)₂ (where t is 1 to 2) where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R²² is alkyl, haloalkyl, cycloalkyl, cycloalkylalkyl, aryl, aralkyl, heterocyclyl, heterocyclylalkyl, heteroaryl or heteroarylalkyl.

“Aryl” refers to a hydrocarbon ring system radical comprising hydrogen, 6 to 18 carbon atoms and at least one aromatic ring. For purposes of this disclosure, the aryl radical may be a monocyclic, bicyclic, tricyclic or tetracyclic ring system, which may included fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group may be optionally substituted by one or more substituents independently selected from the group consisting of alkyl, alkenyl, halo, haloalkyl, haloalkenyl, cyano, nitro, aryl, aralkyl, cycloalkyl, cycloalkylalkyl, heterocyclyl, heterocyclylalkyl, heteroaryl, heteroarylalkyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—N(R²⁰)C(O)OR²², —R²¹—N(R²⁰)C(O)R²², —R²¹—N(R²⁰)S(O)ₜR²² (where t is 1 to 2), —R²¹—N═C(OR²⁰)R²⁰, —R²¹—S(O)ₜOR²² (where t is 1 to 2), —R²¹—S(O)ₚR²² (where p is 0 to 2), and —R²¹—S(O)ₜN(R²⁰)₂ (where t is 1 to 2) where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; each R²¹ is independently a direct bond or a straight or branched alkylene or alkenylene chain; and each R²² is alkyl, haloalkyl, cycloalkyl, cycloalkylalkyl, aryl, aralkyl, heterocyclyl, heterocyclylalkyl, heteroaryl or heteroarylalkyl.

“Aralkyl” or “arylalkyl” refers to a radical of the formula —R_{b}—R_{c} where R_{b} is an alkylene chain as defined above and R_{c} is one or more aryl radicals as defined above, for example, benzyl, diphenylmethyl and the like. The alkylene chain part of the aralkyl radical may be optionally substituted as described above for an alkylene chain. The aryl part of the aralkyl radical may be optionally substituted as described above for an aryl group.

“Aralkenyl” refers to a radical of the formula —R_{d}—R_{c} where R_{d} is an alkenylene chain as defined above and R_{c} is one or more aryl radicals as defined above. The aryl part of the aralkenyl radical may be optionally substituted as described above for an aryl group. The alkenylene chain part of the aralkenyl radical may be optionally substituted as defined above for an alkenylene group.

“Cycloalkyl” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptly, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group may be optionally substituted by one or more substituents independently selected from the group consisting of alkyl, alkenyl, halo, haloalkyl, haloalkenyl, cyano, nitro, oxo, aryl, aralkyl, cycloalkyl, cycloalkylalkyl, heterocyclyl, heterocyclylalkyl, heteroaryl, heteroarylalkyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—N(R²⁰)C(O)OR²², —R²¹—N(R²⁰)C(O)R²², —R²¹—N(R²⁰)S(O)ₜR²² (where t is 1 to 2), —R²¹—N═C(OR²⁰)R²⁰, —R²¹—S(O)ₜOR²² (where t is 1 to 2), —R²¹—S(O)ₚR²² (where p is 0 to 2), and —R²¹—S(O)ₜN(R²⁰)₂ (where t is 1 to 2) where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; each R²¹ is independently a direct bond or a straight or branched alkylene or alkenylene chain; and each R²² is alkyl, haloalkyl, cycloalkyl, cycloalkylalkyl, aryl, aralkyl, heterocyclyl, heterocyclylalkyl, heteroaryl or heteroarylalkyl.

“Cycloalkylalkyl” refers to a radical of the formula —R_{b}R_{g} where R_{b} is an alkylene chain as defined above and R_{g} is a cycloalkyl radical as defined above. The alkylene chain and the cycloalkyl radical may be optionally substituted as defined above.

“Fused” refers to any ring system described herein which is fused to an existing ring structure in the compounds of the disclosure. When the fused ring system is a heterocyclyl or a heteroaryl, any carbon in the existing ring structure which becomes part of the fused ring system may be replaced with a nitrogen.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like. The alkyl part of the haloalkyl radical may be optionally substituted as defined above for an alkyl group.

“Haloalkoxy” refers to a radical having the following formula —OR^{a} where R^{a} is an haloalkyl radical as defined above.

“Haloalkenyl” refers to an alkenyl radical, as defined above, that is substituted by one or more halo radicals, as defined above. The alkenyl part of the haloalkyl radical may be optionally substituted as defined above for an alkenyl group.

“Cyanoalkyl” refers to an alkyl radical, as defined above, that is substituted by one or more cyano radicals (i.e., —CN). The alkyl part of the cyanoalkyl radical may be optionally substituted as defined above for an alkyl group.

“Hydroxyalkyl” refers to an alkyl radical, as defined above, that is substituted by one or more hydroxy radicals (i.e., —OH). The alkyl part of the hydroxyalkyl radical may be optionally substituted as defined above for an alkyl group.

“Alkoxyalkyl” refers to an alkyl radical, as defined above, that is substituted by one or more alkoxy radicals. The alkyl part of the hydroxyalkyl radical may be optionally substituted as defined above for an alkyl group.

“Haloalkoxyalkyl” refers to an alkyl radical, as defined above, that is substituted by one or more haloalkoxy radicals. The alkyl part of the hydroxyalkyl radical may be optionally substituted as defined above for an alkyl group.

“Heterocyclyl” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, dioxinyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trioxanyl, trithianyl, triazinanyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted by one or more substituents selected from the group consisting of alkyl, alkenyl, halo, haloalkyl, haloalkenyl, cyano, oxo, thioxo, nitro, aryl, aralkyl, cycloalkyl, cycloalkylalkyl, heterocyclyl, heterocyclylalkyl, heteroaryl, heteroarylalkyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—N(R²⁰)C(O)OR²², —R²¹—N(R²⁰)C(O)R²², —R²¹—N(R²⁰)S(O)ₜR²² (where t is 1 to 2), —R²¹—N═C(OR²⁰)R²⁰, —R²¹—S(O)ₜOR²² (where t is 1 to 2), —R²¹—S(O)ₚR²² (where p is 0 to 2), and —R²¹—S(O)ₜN(R²⁰)₂ (where t is 1 to 2) where each R²⁰ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R²¹ is independently a direct bond or a straight or branched alkylene or alkenylene chain; and each R²² is alkyl, alkenyl, haloalkyl, cycloalkyl, cycloalkylalkyl, aryl, aralkyl, heterocyclyl, heterocyclylalkyl, heteroaryl or heteroarylalkyl.

“O-heterocyclyl” refers to a heterocycyl radical as defined above containing at least one oxygen atom and no nitrogen atom. An O-heterocyclyl radical may be optionally substituted as described above for heterocyclyl radicals.

“Heterocyclylalkyl” refers to a radical of the formula —R_{b}Rₕ where R_{b} is an alkylene chain as defined above and Rₕ is a heterocyclyl radical as defined above, and if the heterocyclyl is a nitrogen-containing heterocyclyl, the heterocyclyl may be attached to the alkyl radical at the nitrogen atom. The alkylene chain of the heterocyclylalkyl radical may be optionally substituted as defined above for an alkyene chain. The heterocyclyl part of the heterocyclylalkyl radical may be optionally substituted as defined above for a heterocyclyl group.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of this disclosure, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, benzoxazolinonyl, benzimidazolthionyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, pteridinonyl, purinyl, pyrrolyl, pyrazolyl, pyridinyl, pyridinonyl, pyrazinyl, pyrimidinyl, pryrimidinonyl, pyridazinyl, pyrrolyl, pyrido[2,3-d]pyrimidinonyl, quinazolinyl, quinazolinonyl, quinoxalinyl, quinoxalinonyl, quinolinyl, isoquinolinyl, tetrahydroquinolinyl, thiazolyl, thiadiazolyl, thieno[3,2-d]pyrimidin-4-onyl, thieno[2,3-d]pyrimidin-4-onyl, triazolyl, tetrazolyl, triazinyl, and thiophenyl (i.e. thienyl). Unless stated otherwise specifically in the specification, a heteroaryl group may be optionally substituted by one or more substituents selected from the group consisting of alkyl, alkenyl, halo, haloalkyl, haloalkenyl, cyano, oxo, thioxo, nitro, thioxo, aryl, aralkyl, cycloalkyl, cycloalkylalkyl, heterocyclyl, heterocyclylalkyl, heteroaryl, heteroarylalkyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—N(R²⁰)C(O)OR²², —R²¹—N(R²⁰)C(O)R²², —R²¹—N(R²⁰)S(O)ₜR²² (where t is 1 to 2), —R²¹—N═C(OR²⁰)R²⁰, —R²¹—S(O)ₜOR²² (where t is 1 to 2), —R²¹—S(O)ₚR²² (where p is 0 to 2), and —R²¹—S(O)ₜN(R²⁰)₂ (where t is 1 to 2) where each R²⁰ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R²¹ is independently a direct bond or a straight or branched alkylene or alkenylene chain; and each R²² is alkyl, alkenyl, haloalkyl, cycloalkyl, cycloalkylalkyl, aryl, aralkyl, heterocyclyl, heterocyclylalkyl, heteroaryl or heteroarylalkyl.

“N-heteroaryl” refers to a heteroaryl radical as defined above containing at least one nitrogen. An N-heteroaryl radical may be optionally substituted as described above for heteroaryl radicals.

“Heteroarylalkyl” refers to a radical of the formula —R_{b}Rᵢ where R_{b} is an alkylene chain as defined above and Rᵢ is a heteroaryl radical as defined above. The heteroaryl part of the heteroarylalkyl radical may be optionally substituted as defined above for a heteroaryl group. The alkylene chain part of the heteroarylalkyl radical may be optionally substituted as defined above for an alkylene chain.

“Prodrugs” is meant to indicate a compound that may be converted under physiological conditions or by solvolysis to a biologically active compound of the disclosure. Thus, the term “prodrug” refers to a metabolic precursor of a compound of the disclosure that is pharmaceutically acceptable. A prodrug may be inactive when administered to a subject in need thereof, but is converted in vivo to an active compound of the disclosure. Prodrugs are typically rapidly transformed in vivo to yield the parent compound of the disclosure, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., “Pro-drugs as Novel Delivery Systems,” A.C.S. Symposium Series, Vol. 14, and in Bioreversible Carriers in Drug Design, Ed. Edward B. Roche, American Pharmaceutical Association and Pergamon Press, 1987, both of which are incorporated in full by reference herein.

The term “prodrug” is also meant to include any covalently bonded carriers, which release the active compound of the disclosure in vivo when such prodrug is administered to a mammalian subject. Prodrugs of a compound of the disclosure may be prepared by modifying functional groups present in the compound of the disclosure in such a way that the modifications are cleaved, either in routine manipulation or in vivo, to the parent compound of the disclosure. Prodrugs include compounds of the disclosure wherein a hydroxy, amino or mercapto group is bonded to any group that, when the prodrug of the compound of the disclosure is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in the compounds of the disclosure and the like.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets, (e.g., cats, dogs, swine, cattle, sheep, goats, horses, rabbits), and non-domestic animals such as wildelife and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution (“unsubstituted). When a functional group is described as “optionally substituted,” and in turn, substituents on the functional group are also “optionally substituted” and so on, for the purposes of this disclosure, such iterations are limited to five, preferably such iterations are limited to two.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the disclosure. As used herein, the term “solvate” refers to an aggregate or solid form that comprises one or more molecules of a compound of the disclosure with one or more molecules of solvent. The solvent may be water, in which case the solvate may be a hydrate. Alternatively, the solvent may be an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. The compound of the disclosure may be true solvates, while in other cases; the compound of the disclosure may merely retain adventitious water or be a mixture of water plus some adventitious solvent.

A “pharmaceutical composition” refers to a formulation of a compound of the disclosure and a medium generally accepted in the art for the delivery of the biologically active compound to mammals, e.g., humans. Such a medium includes all pharmaceutically acceptable carriers, diluents or excipients therefor.

“Seizure disorders” refers to seizures and disorders associated with seizures such as partial onset (focal) seizures, photosensitive epilepsy, self-induced syncope, intractable epilepsy, Angelman syndrome, benign rolandic epilepsy, CDKL5 disorder, childhood and juvenile absence epilepsy, Dravet syndrome, frontal lobe epilepsy, Glut1 deficiency syndrome, hypothalamic hamartoma, infantile spasms/West's syndrome, juvenile myoclonic epilepsy, Landau-Kleffner syndrome, Lennox-Gastaut syndrome (LGS), epilepsy with myoclonic-absences, Ohtahara syndrome, Panayiotopoulos syndrome, PCDH19 epilepsy, progressive myoclonic epilepsies, Rasmussen's syndrome, ring chromosome 20 syndrome, reflex epilepsies, temporal lobe epilepsy, Lafora progressive myoclonus epilepsy, neurocutaneous syndromes, tuberous sclerosis complex, early infantile epileptic encephalopathy, early onset epileptic encephalopathy, generalized epilepsy with febrile seizures +, Rett syndrome, multiple sclerosis, Alzheimer's disease, autism, ataxia, hypotonia and paroxysmal dyskinesia. Preferably, the term “seizure disorder” refers to partial onset (focal) epilepsy.

“Therapeutically effective amount” refers to a range of amounts of a compound of the disclosure, which, upon administration to a human, treats, ameliorates or prevents a seizure disorder, preferably epilepsy, in the human, or exhibits a detectable therapeutic or preventative effect in the human having a seizure disorder. The effect is detected by, for example, a reduction in seizures (frequency) or by the severity of seizures (quality). The precise therapeutically effective amount for a given human will depend upon the human's size and health, the nature and extent of the seizure disorder, the presence of any concomitant medications, and other variables known to those of skill in the art. The therapeutically effective amount for a given situation is determined by routine experimentation and is within the judgment of the clinician.

“Treatment” refers to therapeutic applications to slow or stop progression of a seizure disorder, prophylactic application to prevent development of a seizure disorder, and/or reversal of a seizure disorder. Reversal of a seizure disorder differs from a therapeutic application which slows or stops a seizure disorder in that with a method of reversing, not only is progression of a seizure disorder completely stopped, cellular behavior is moved to some degree toward a normal state that would be observed in the absence of the seizure disorder.

“Treating” or “treatment” as used herein covers the treatment of the disease or condition of interest in a mammal, preferably a human, having the disease or condition of interest, and includes:

(a) preventing the disease or condition from occurring in a mammal, in particular, when such mammal is predisposed to the condition but has not yet been diagnosed as having it;

(b) inhibiting the disease or condition, i.e., arresting its development;

(c) relieving (or ameliorating) the disease or condition, i.e., causing regression of the disease or condition; or

(d) relieving (or ameliorating) the symptoms resulting from the disease or condition, i.e., relieving a seizure disorder without addressing the underlying disease or condition.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of this disclosure may contain at least one asymmetric carbon atom and thus may exist as racemates, enantiomers, and/or diastereoisomers. For the present disclosure, the words diastereomer and diastereoisomer and related terms are equivalent and interchangeable. Unless otherwise indicated, this disclosure includes all enantiomeric and diastereoisomeric forms of the compounds of formula (I). Pure stereoisomers, mixtures of enantiomers and/or diastereoisomers, and mixtures of different compounds of the disclosure are included herein. Thus, compounds of formula (I) or (II) may occur as racemates, racemic or diastereoisomeric mixtures and as individual diastereoisomers, or enantiomers, unless a specific stereoisomer enantiomer or diastereoisomer is identified, with all isomeric forms being included in the present disclosure. For this disclosure, a racemate or racemic mixture implies a 50:50 mixture of stereoisomers only. Other enantiomerically or diastereomerically enriched mixtures of varying ratios of stereoisomers are also contemplated.

“Enantiomers” refer to asymmetric molecules that can exist in two different isomeric forms which have different configurations in space. Other terms used to designate or refer to enantiomers include “stereoisomers” (because of the different arrangement or stereochemistry around the chiral center; although all enantiomers are stereoisomers, not all stereoisomers are enantiomers) or “optical isomers” (because of the optical activity of pure enantiomers, which is the ability of different pure enantiomers to rotate plane-polarized light in different directions). Because they do not have a plane of symmetry, enantiomers are not identical with their mirror images; molecules which exist in two enantiomeric forms are chiral, which means that they can be regarded as occurring in “left” and “right” handed forms. The most common cause of chirality in organic molecules is the presence of a tetrahedral carbon bonded to four different substituents or groups. Such a carbon is referred to as a chiral center, or stereogenic center.

Enantiomers have the same empirical chemical formula, and are generally chemically identical in their reactions, their physical properties, and their spectroscopic properties. However, enantiomers show different chemical reactivity toward other asymmetric compounds, and respond differently toward asymmetric physical disturbances. The most common asymmetric disturbance is polarized light.

An enantiomer can rotate plane-polarized light; thus, an enantiomer is optically active. Two different enantiomers of the same compound will rotate plane-polarized light in the opposite direction; thus, the light can be rotated to the left or counterclockwise for a hypothetical observer (this is levarotatory or “I”, or minus or “−”) or it can be rotated to the right or clockwise (this is dextrorotatory or “d” or plus or “+”). The sign of optical rotation (+) or (−), is not related to the R,S designation. A mixture of equal amounts of two chiral enantiomers is called a racemic mixture, or racemate, and is denoted either by the symbol (+/−) or by the prefix “d,l” to indicate a mixture of dextrorotatory and levorotatory forms. Racemates or racemic mixtures show zero optical rotation because equal amounts of the (+) and (−) forms are present. In general, the presence of a single enantiomer rotates polarized light in only one direction; thus, a single enantiomer is referred to as optically pure.

The designations “R” and “S” are used to denote the three-dimensional arrangement of atoms (or the configuration) of the stereogenic center. The designations may appear as a prefix or as a suffix; they may or may not be separated from the enantiomer name by a hyphen; they may or may not be hyphenated; and they may or may not be surrounded by parentheses. A method for determining the designation is to refer to the arrangement of the priority of the groups at the stereogenic center when the lowest priority group is oriented away from a hypothetical observer: If the arrangement of the remaining three groups from the higher to the lower priority is clockwise, the stereogenic center has an “R” configuration; if the arrangement is counterclockwise, the stereogenic center has an “S” configuration.

“Resolution” or “resolving” when used in reference to a racemic compound or mixture refers to the separation of a racemate into its two enantiomeric forms (i.e., (+) and (−); (R) and (S) forms).

“Enantiomeric excess” or “ee” refers to a product wherein one enantiomer is present in excess of the other, and is defined as the absolute difference in the mole fraction of each enantiomer. Enantiomeric excess is typically expressed as a percentage of an enantiomer present in a mixture relative to the other enantiomer. For purposes of this disclosure, the (S)-enantiomer of a compound prepared by the methods disclosed herein is considered to be “substantially free” of the corresponding (R)-enantiomer when the (S)-enantiomer is present in enantiomeric excess of greater than 80%, preferably greater than 90%, more preferably greater than 95% and most preferably greater than 99%.

Certain compounds have been labeled with “P1”, “P2”, et seq. or “D1”, “D2”, et seq. This demarcation indicates a compound is a first eluting peak (i.e., P1) from a chiral separation technique and does not necessarily indicate a specific stereochemistry.

A “tautomer” refers to a proton shift from one atom of a molecule to another atom of the same molecule. The present disclosure includes tautomers of any compound of formula (I) or (II) as described herein.

“Isotopologue” refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound will depend upon a number of factors including the isotopic purity of deuterated reagents used to make the compound and the efficiency of incorporation of deuterium in the various synthesis steps used to prepare the compound. The relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The use of parentheses and brackets in substituent groups may be used herein to conserve space. Accordingly, the use of parenthesis in a substituent group indicates that the group enclosed within the parentheses is attached directly to the atom preceding the parenthesis. The use of brackets in a substituent group indicates that the group enclosed within the brackets is also attached directly to the atom preceding the parenthesis.

For example, a compound of formula (I) or (II) wherein a compound having the following structure:

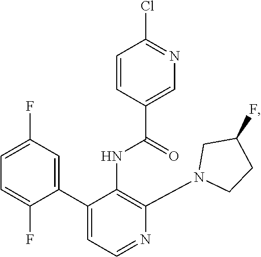 [SMILES: O=C(Nc1c(-c2cc(F)ccc2F)ccnc1N1CC[C@H](F)C1)c1ccc(Cl)nc1]

is named herein as (S)-6-chloro-N-(4-(2,5-difluorophenyl)-2-(3-fluoropyrrolidin-1-yl)pyridin-3-yl)nicotinamide.

### Compounds

One embodiment of the disclosure is compounds of formula (I) or (II), as set forth above in the Brief Summary, as individual stereoisomers, enantiomers, or tautomers thereof or as mixtures thereof; or pharmaceutically acceptable salts, solvates, or prodrugs thereof.

One embodiment provides a compound of formula (I):

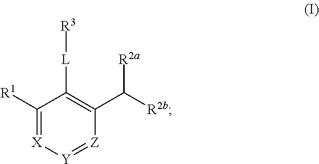

wherein:
X, Y, and Z are each independently N or CR^{1b}, provided that at least one and no more than two of X, Y, and Z are N;
L is a direct bond, —NR⁴C(═O)—, or —C(═O)NR⁴—;
R¹ is methoxy, —R⁵N(R⁶)₂, alkenyl, or has one of the following structures:

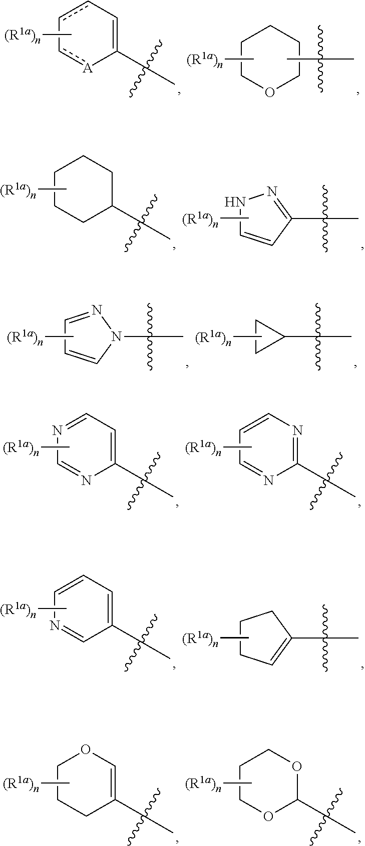

wherein:
each [symbol] is independently a single or double bond such that all valences are satisfied;
each R^{1a} is independently alkyl, halo, haloalkyl, —R⁵OR⁶, —R⁵N(R⁶)₂, —R⁵OC(═O)R⁶, optionally substituted cycloalkyl, or —R⁵C(═O)OR⁶;
A is O, N, or C;
each R^{1b} is independently hydrogen, halo, alkyl, or haloalkyl;
R^{2a} and R^{2b}, together with the carbon to which they are attached, form an optionally substituted cycloalkyl, an optionally substituted cycloalkenyl, an optionally substituted aryl, an optionally substituted heterocyclyl, or an optionally substituted heteroaryl;
   or R^{2a} is hydrogen or alkyl and R^{2b} is an optionally substituted heterocyclyl or an optionally substituted cycloalkyl;
   or R^{2a} and R^{2b} are both alkyl;
   or R^{2a} is alkyl and R^{2b} is haloalkoxy;
R³ is alkyl, cyanoalkyl, —R⁵OR⁶, —R⁵N(R⁶)₂, an optionally substituted cycloalkyl, an optionally substituted aryl, an optionally substituted heterocyclyl, an optionally substituted heteroaryl, an optionally substituted cycloalkylalkyl, an optionally substituted heterocyclylalkyl;
R⁴ is hydrogen or alkyl;
each R⁵ is independently a direct bond or an optionally substituted alkylene chain;
each R⁶ is independently hydrogen, alkyl, haloalkyl, optionally substituted cycloalkyl, or optionally substituted arylalkyl;
or two R⁶'s, together with the nitrogen to which they are both attached, form an optionally substituted heterocyclyl; and
n is 0, 1, 2, 3, 4, or 5,
as a stereoisomer, enantiomer, or tautomer thereof or a mixture thereof; or a pharmaceutically acceptable salt, solvate, isotopologue, or prodrug thereof.

In some embodiments, the compound has the following formula (Ia):

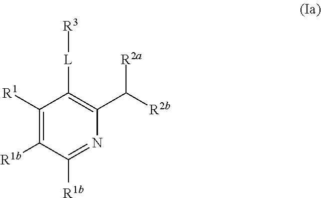

and R¹, R^{1b}, R^{2a}, R^{2b}, L and R³ are as defined in the Brief Description,
as a stereoisomer, enantiomer, or tautomer thereof or a mixture thereof; or a pharmaceutically acceptable salt, solvate, isotopologue, or prodrug thereof.

In certain embodiments, the compound has the following formula (Ia1):

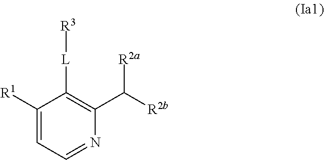

and R¹, R^{1b}, R^{2a}, R^{2b}, L and R³ are as defined in the Brief Description,
as a stereoisomer, enantiomer, or tautomer thereof or a mixture thereof; or a pharmaceutically acceptable salt, solvate, isotopologue, or prodrug thereof.

In some embodiments, the compound has the following formula (Ib):

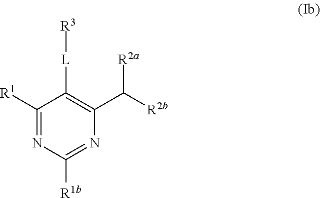

and R¹, R^{1b}, R^{2a}, R^{2b}, L and R³ are as defined in the Brief Description,
as a stereoisomer, enantiomer, or tautomer thereof or a mixture thereof; or a pharmaceutically acceptable salt, solvate, isotopologue, or prodrug thereof.

In certain embodiments, the compound has the following formula (Ib1):

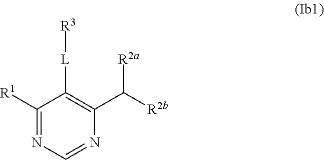

and R¹, R^{1b}, R^{2a}, R^{2b}, L and R³ are as defined in the Brief Description,
as a stereoisomer, enantiomer, or tautomer thereof or a mixture thereof; or a pharmaceutically acceptable salt, solvate, isotopologue, or prodrug thereof.

In some embodiments, the compound has the following formula (Ic):

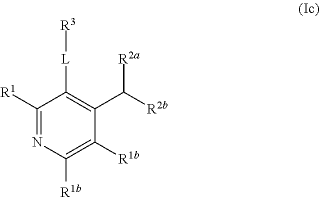

and R¹, R^{1b}, R^{2a}, R^{2b}, L and R³ are as defined in the Brief Description,
as a stereoisomer, enantiomer, or tautomer thereof or a mixture thereof; or a pharmaceutically acceptable salt, solvate, isotopologue, or prodrug thereof.

In certain embodiments, the compound has the following formula (Ic1):

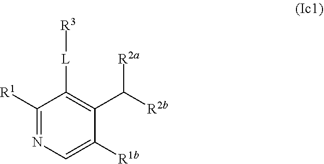

and R¹, R^{1b}, R^{2a}, R^{2b}, L and R³ are as defined in the Brief Description,
as a stereoisomer, enantiomer, or tautomer thereof or a mixture thereof; or a pharmaceutically acceptable salt, solvate, isotopologue, or prodrug thereof.

In some embodiments, the compound has the following formula (Id):

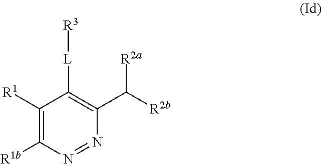

and R¹, R^{1b}, R^{2a}, R^{2b}, L and R³ are as defined in the Brief Description,
as a stereoisomer, enantiomer, or tautomer thereof or a mixture thereof; or a pharmaceutically acceptable salt, solvate, isotopologue, or prodrug thereof.

In certain embodiments, the compound has the following formula (Ie):

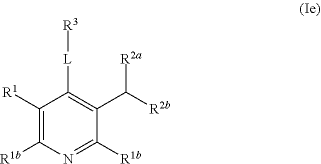

and R¹, R^{1b}, R^{2a}, R^{2b}, L and R³ are as defined in the Brief Description,
   as a stereoisomer, enantiomer, or tautomer thereof or a mixture thereof; or a pharmaceutically acceptable salt, solvate, isotopologue, or prodrug thereof.

One embodiment provides a compound of formula (II):

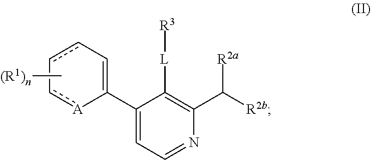

wherein:
each [symbol] is independently a single or double bond;
A is O, N, or C;
L is a direct bond, —NR⁴C(═O)—, or —C(═O)NR⁴—;
each R¹ is independently alkyl, halo, haloalkyl, —R⁵OR⁶, —R⁵N(R⁶)₂, —R⁵OC(═O)R⁶, or —R⁵C(═O)OR⁶;
R^{2a} and R^{2b}, together with the carbon to which they are attached, form an optionally substituted cycloalkyl, an optionally substituted cycloalkenyl, an optionally substituted aryl, an optionally substituted heterocyclyl, or an optionally substituted heteroaryl;
R³ is an optionally substituted cycloalkyl, an optionally substituted aryl, an optionally substituted heterocyclyl, or an optionally substituted heteroaryl;
R⁴ is hydrogen or alkyl;
each R⁵ is independently a direct bond or an optionally substituted alkylene chain;
each R⁶ is independently hydrogen, alkyl, haloalkyl, or optionally substituted cycloalkyl;
or two R⁶'s, together with the nitrogen to which they are both attached, form an optionally substituted heterocyclyl; and
n is 0, 1, 2, 3, 4, or 5,
as a stereoisomer, enantiomer, or tautomer thereof or a mixture thereof; or a pharmaceutically acceptable salt, solvate, or prodrug thereof.

Another embodiment provides compound of formula (IIa):

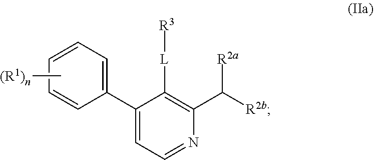

wherein:
L is —NR⁴C(═O)— or —C(═O)NR⁴—;
   each R¹ is independently alkyl, halo, haloalkyl, —R⁵OR⁶, —R⁵N(R⁶)₂, —R⁵OC(═O)R⁶, or —R⁵C(═O)OR⁶;
R^{2a} and R^{2b}, together with the carbon to which they are attached, form an optionally substituted cycloalkyl, an optionally substituted cycloalkenyl, an optionally substituted aryl, an optionally substituted heterocyclyl, or an optionally substituted heteroaryl;
R³ is an optionally substituted cycloalkyl, an optionally substituted aryl, an optionally substituted heterocyclyl, or an optionally substituted heteroaryl;
R⁴ is hydrogen or alkyl;
each R⁵ is independently a direct bond or an optionally substituted alkylene chain;
each R⁶ is independently hydrogen, alkyl, haloalkyl, or optionally substituted cycloalkyl;
or two R⁶'s, together with the nitrogen to which they are both attached, form an optionally substituted heterocyclyl; and
n is 0, 1, 2, 3, 4, or 5,
   as a stereoisomer, enantiomer, or tautomer thereof or a mixture thereof; or a pharmaceutically acceptable salt, solvate, or prodrug thereof.

In some embodiments, one occurrence of [symbol] is a single bond. In certain other embodiments, one occurrence of [symbol] is a double bond. In some other embodiments, one occurrence of [symbol] is a single bond and the other occurrence of [symbol] is a double bond. In some other specific embodiments, both occurrences of [symbol] are single bonds. In some specific embodiments, both occurrences of [symbol] are double bonds.

In some embodiments, A is N, or C. In some embodiments, A is O. In some embodiments, A is C. In certain specific embodiments, A is N.

In some specific embodiments, both occurrences of [symbol] are double bonds and A is C.

In some embodiments, L is a direct bond. In certain embodiments, L is —C(═O)NR⁴—. In some specific embodiments, L is —NR⁴C(═O)—. In some more specific embodiments, L is —C(═O)NR⁴— or —NR⁴C(═O)—.

In some embodiments, each R¹ is independently alkyl, halo, haloalkyl, —R⁵OR⁶, —R⁵N(R⁶)₂, or —R⁵OC(═O)R⁶. In certain embodiments, each R¹ is independently alkyl, halo, haloalkyl, —R⁵OR⁶, or —R⁵N(R⁶)₂. In some specific embodiments, each R¹ is independently alkyl, halo, haloalkyl, or —R⁵OR⁶. In certain specific embodiments, each R¹ is independently alkyl, halo, or haloalkyl. In some more specific embodiments, each R¹ is independently alkyl or halo. In certain more specific embodiments, each R¹ is independently halo. In some embodiments, each R¹ is independently fluoro and n is 1 or 2. In some embodiments, R¹ is fluoro and n is 1. In some embodiments, R¹ has one of the following structures:

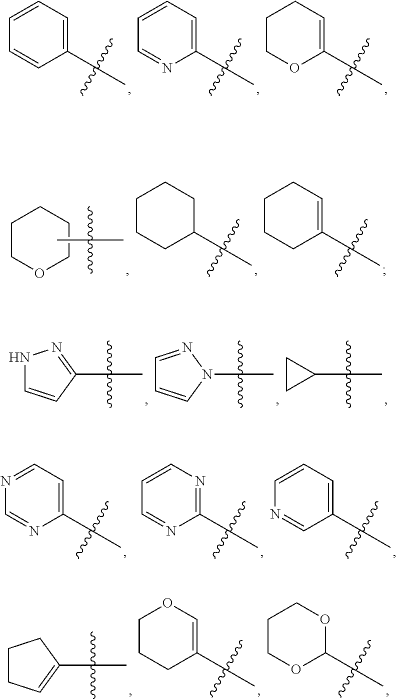

In certain embodiments, R¹ has one of the following structures:

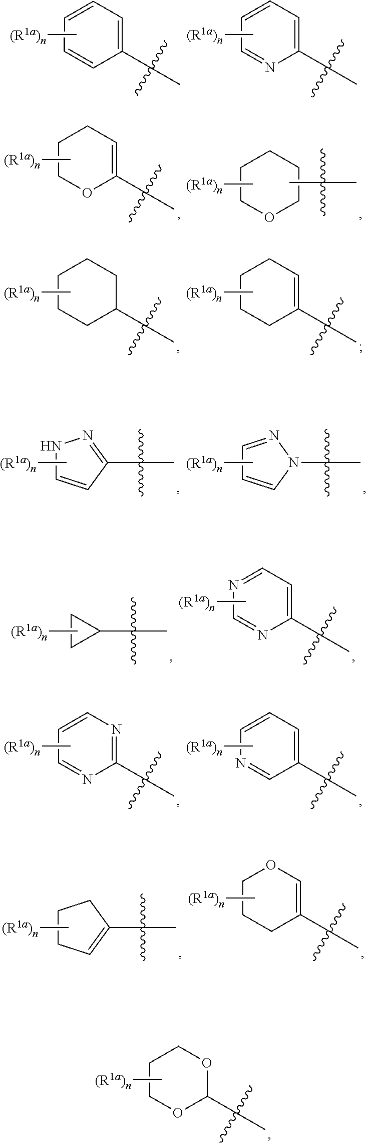

wherein:
n is 1, 2, 3, 4, or 5.

In some embodiments, each R^{1a} is independently methyl, methoxy, trifluoromethyl, fluoro, chloro, or has the following structure:

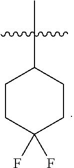 [SMILES: *C1CCC(F)(F)CC1]

In some embodiments, R¹ has one of the following structures:

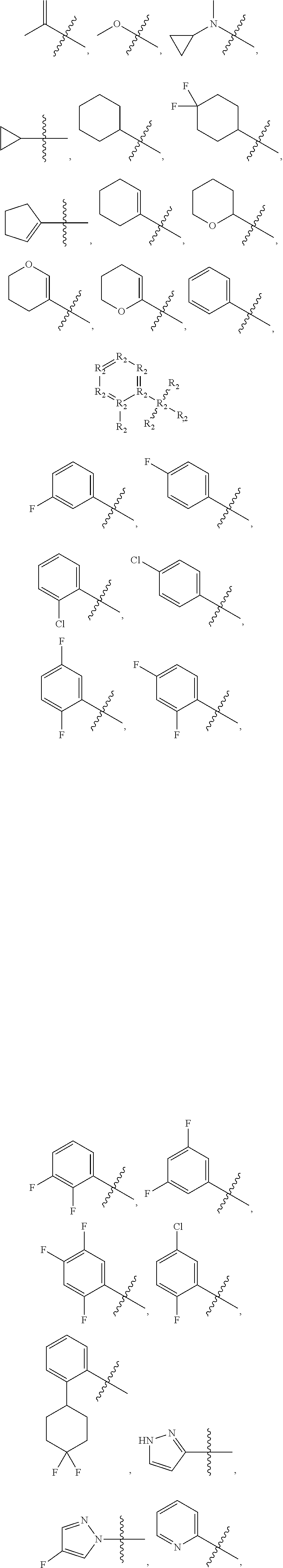

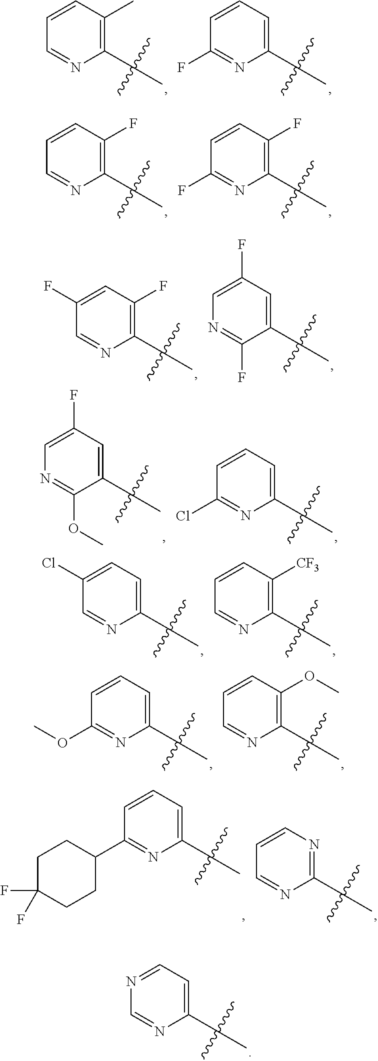

In some embodiments, R¹ has one of the following structures:

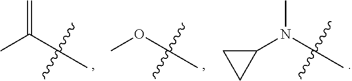

In certain embodiments, R¹ has one of the following structures:

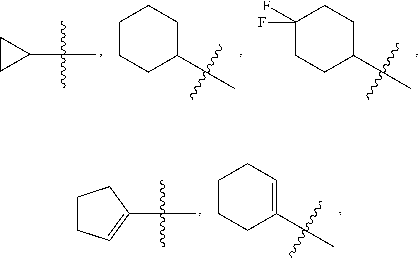

In some embodiments, R¹ has one of the following structures:

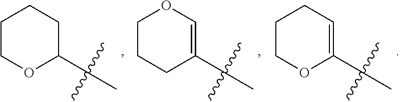 [SMILES: *C1CCCCO1 ; *C1=COCCC1 ; *C1=CCCCO1]

In certain embodiments, R¹ has one of the following structures:

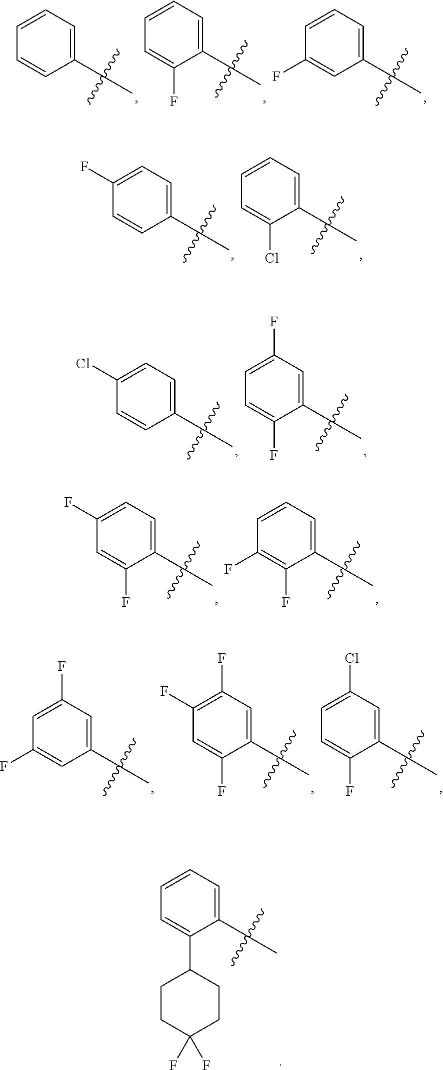

In some embodiments, R¹ has one of the following structures:

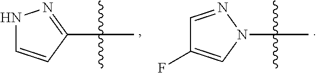 [SMILES: *c1cc[nH]n1 ; *n1cc(F)cn1]

In certain embodiments, R¹ has one of the following structures:

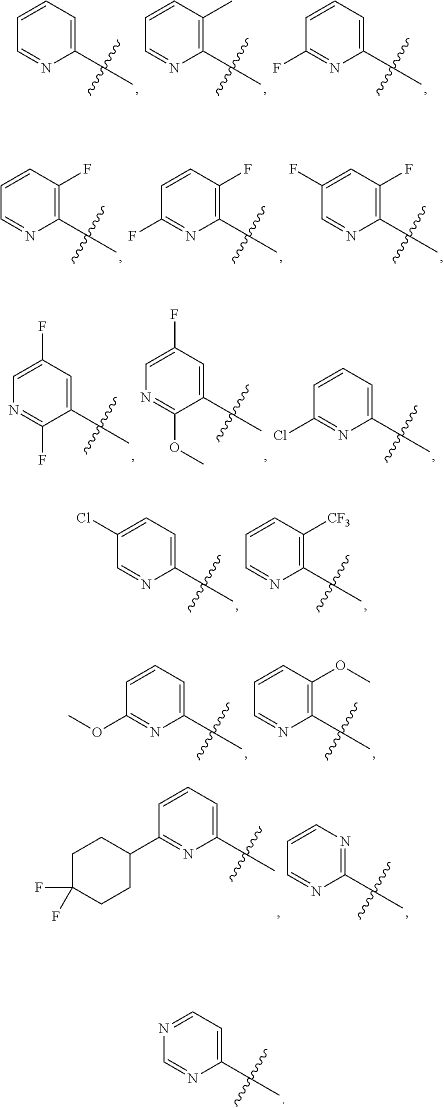

In some embodiments, R^{2a} and R^{2b}, together with the carbon to which they are attached, form an optionally substituted cycloalkyl. In certain embodiments, R^{2a} and R^{2b}, together with the carbon to which they are attached, form an optionally substituted monocyclic, fused, or spirocyclic cycloalkyl. In more specific embodiments, R^{2a} and R^{2b}, together with the carbon to which they are attached, form an optionally substituted cycloalkenyl. In some specific embodiments, R^{2a} and R^{2b}, together with the carbon to which they are attached, form an optionally substituted aryl. In certain embodiments, R^{2a} and R^{2b}, together with the carbon to which they are attached, form an optionally substituted heterocyclyl. In some embodiments, R^{2a} and R^{2b}, together with the carbon to which they are attached, form an optionally substituted N-heterocyclyl. In some specific embodiments, R^{2a} and R^{2b}, together with the carbon to which they are attached, form an optionally substituted monocyclic N-heterocyclyl. In some specific embodiments, R^{2a} and R^{2b}, together with the carbon to which they are attached, form an optionally substituted O-heterocyclyl. In more specific embodiments, R^{2a} and R^{2b}, together with the carbon to which they are attached, form an optionally substituted monocyclic or fused O-heterocyclyl. In some more specific embodiments, R^{2a} and R^{2b}, together with the carbon to which they are attached, form an optionally substituted heteroaryl. In some specific embodiments, R^{2a} and R^{2b}, together with the carbon to which they are attached, form one of the following structures:

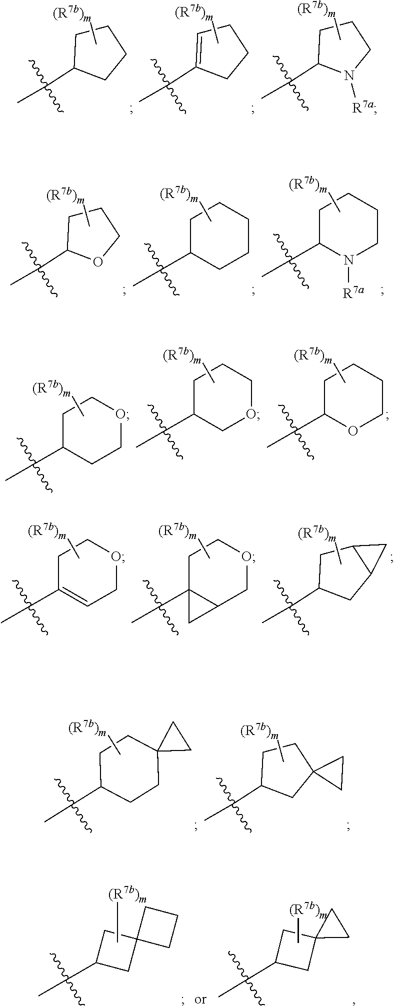

as a stereoisomer, enantiomer, or tautomer thereof or a mixture thereof; or a pharmaceutically acceptable salt, solvate, or prodrug thereof.

In some embodiments, R³ is an optionally substituted cycloalkyl. In certain embodiments, R³ is an optionally substituted aryl. In some specific embodiments, R³ is an optionally substituted heterocyclyl. In certain specific embodiments, R³ is an optionally substituted N-heterocyclyl. In some more specific embodiments, R³ is an optionally substituted heteroaryl. In certain more specific embodiments, R³ is an optionally substituted N-heteroaryl. In some embodiments, R³ is an optionally substituted 5- or 6-membered heteroaryl. In certain embodiments, R³ is an optionally substituted fused bicyclic heteroaryl. In some embodiments, R^{2a} hydrogen or alkyl and R^{2b} is an optionally substituted heterocyclyl or an optionally substituted cycloalkyl. In certain embodiments, R^{2a} and R^{2b} are both alkyl. In some embodiments, R^{2a} is alkyl and R^{2b} is haloalkoxy.

In certain embodiments, R^{2a} and R^{2b}, together with the carbon to which they are attached, form one of the following structures:

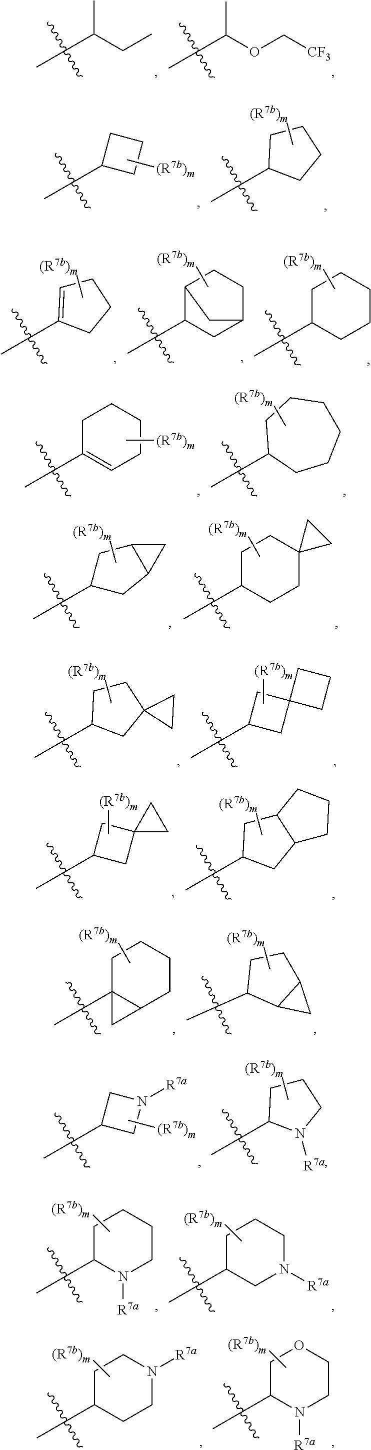

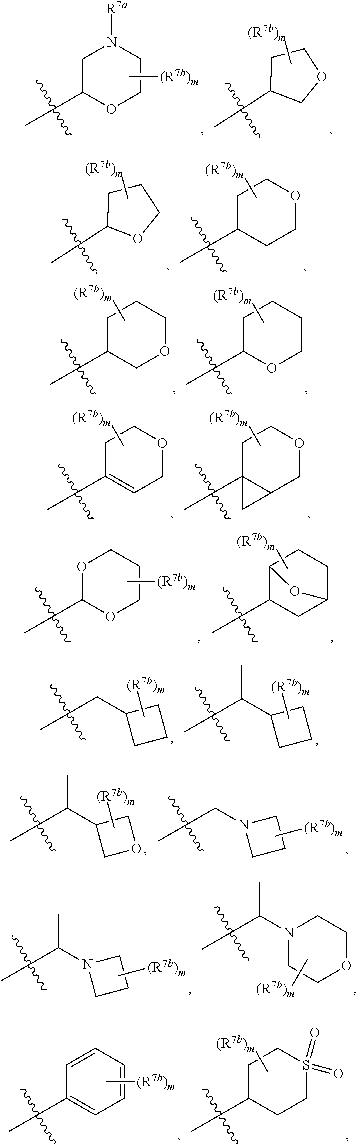

R^{7a} is hydrogen, alkyl, haloalkyl, —R^{7c}C(═O)R^{7d}, —R^{7c}C(═O)OR^{7d}, or heterocyclyl;
each R^{7b} is independently alkyl, halo, haloalkyl, cyano, —R^{7c}OR^{7d}, or —R^{7c}OC(═O)R^{7d}, or two R^{7b}'s, together with the carbon to which they are both attached join to form —C(═O)—;
each R^{7c} is independently a direct bond or an optionally substituted alkylene chain;
each R^{7d} is independently hydrogen, alkyl, haloalkyl, optionally substituted cycloalkyl, or optionally substituted arylalkyl;
or two R^{7d}'s, together with the nitrogen to which they are both attached, form an optionally substituted heterocyclyl; and
m is 0, 1, 2, 3, 4, or 5.

In some embodiments, R^{2a} and R^{2b}, together with the carbon to which they are attached, form one of the following structures:

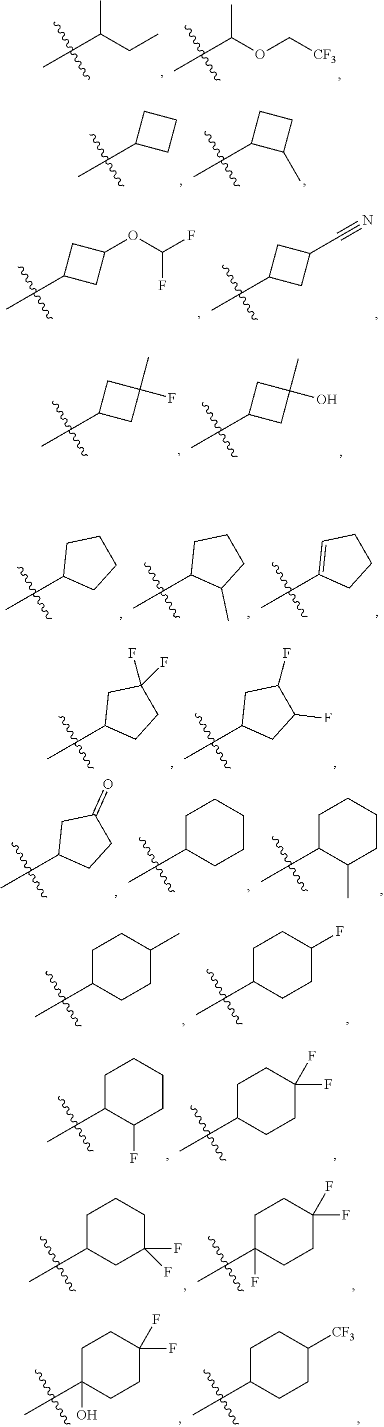

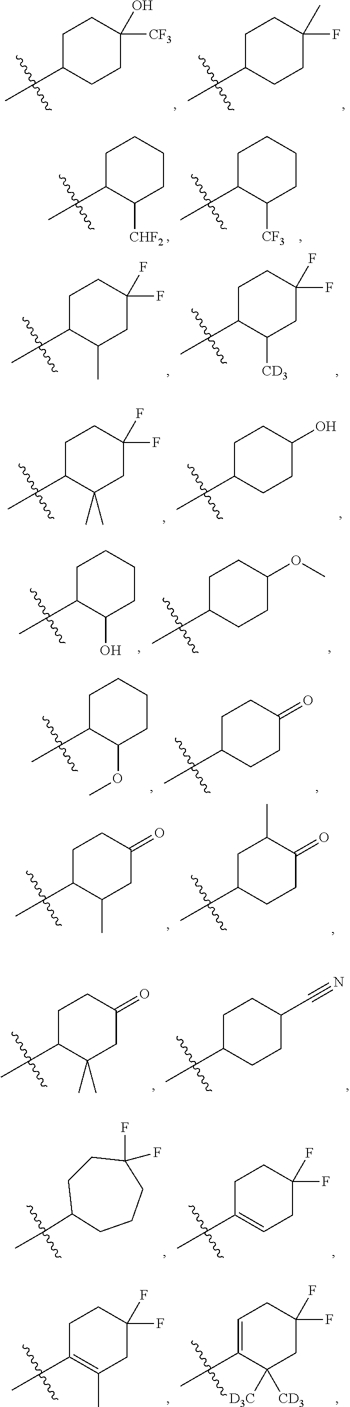

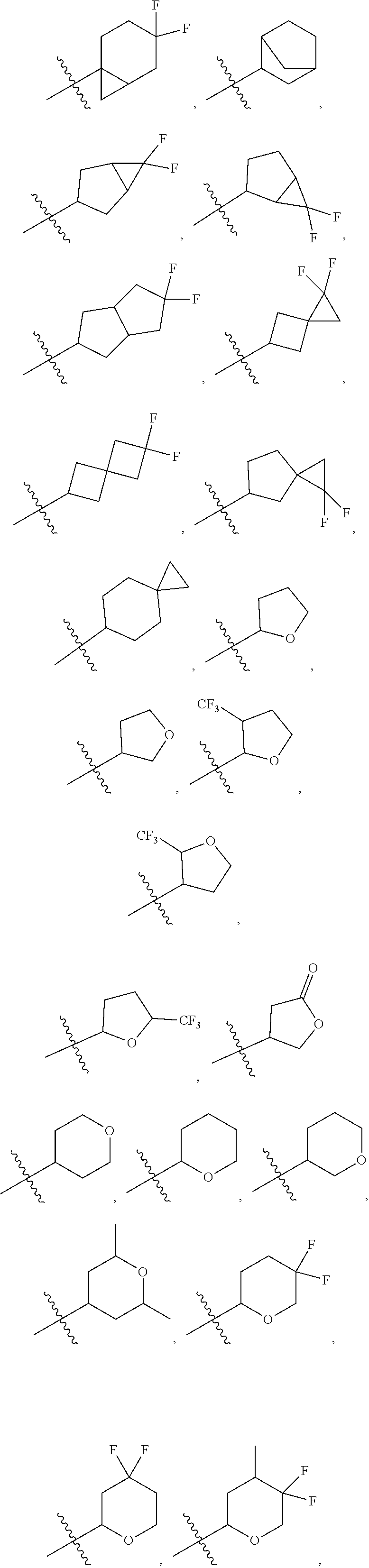

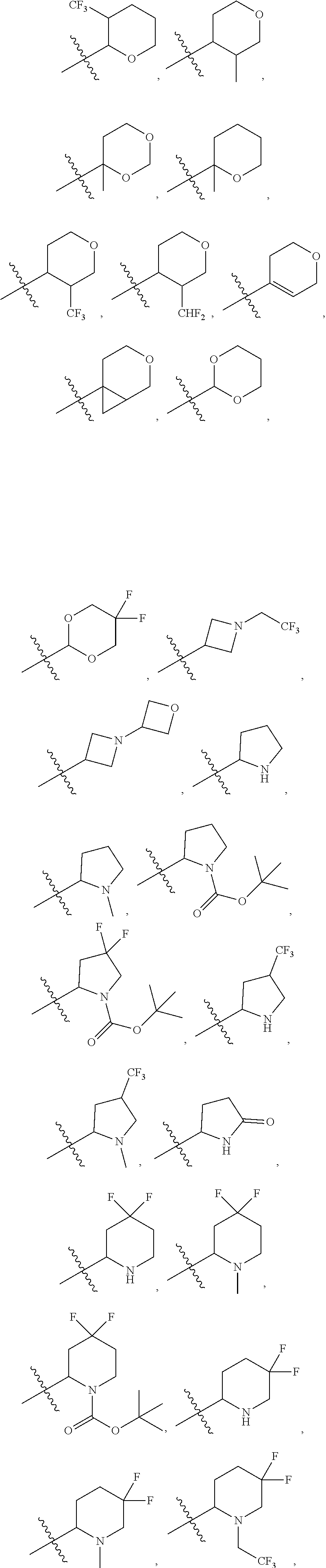

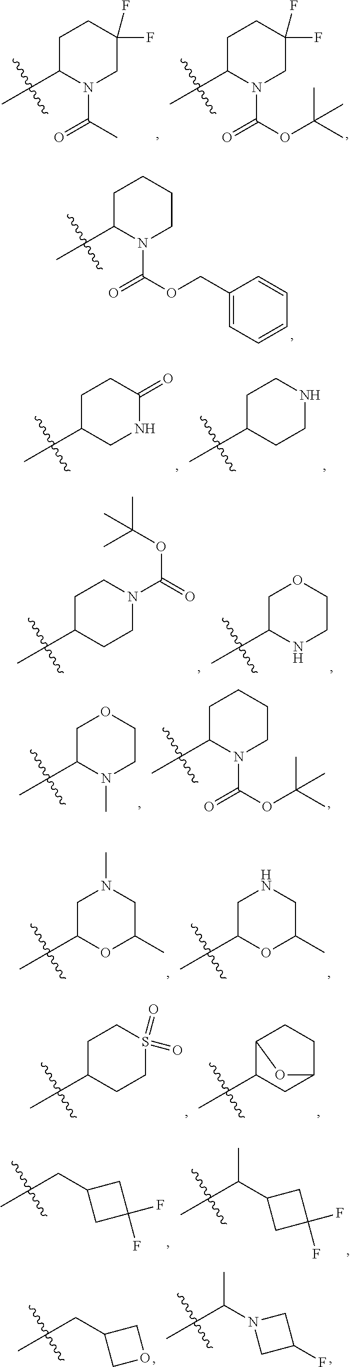

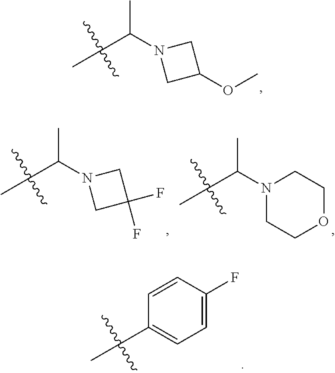

In some embodiments, R^{2a} and R^{2b}, together with the carbon to which they are attached, form one of the following structures:

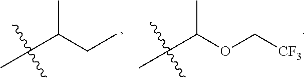

In some embodiments, R^{2a} and R^{2b}, together with the carbon to which they are attached, form one of the following structures:

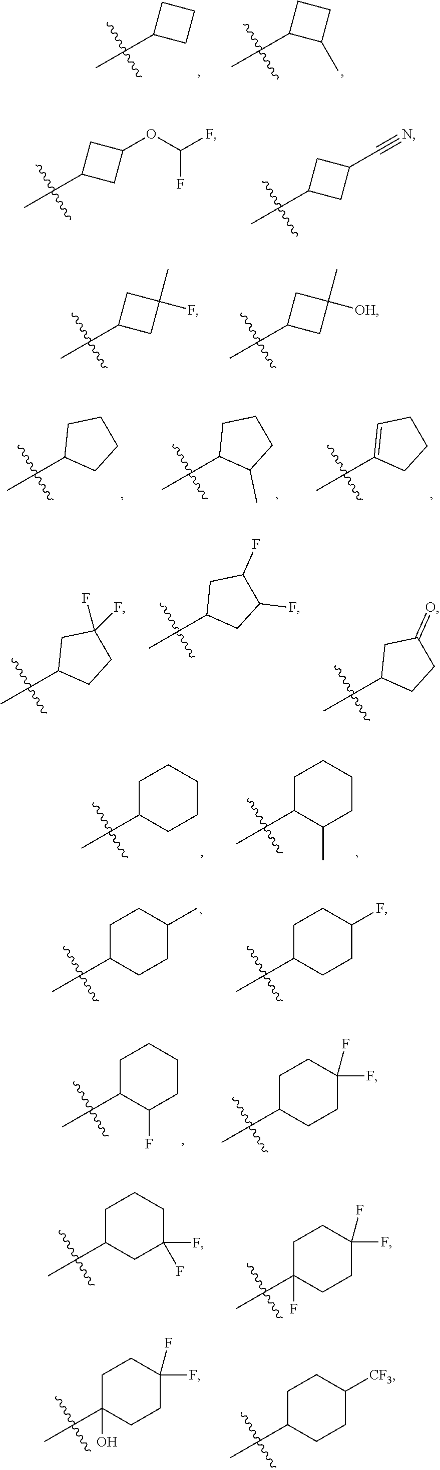

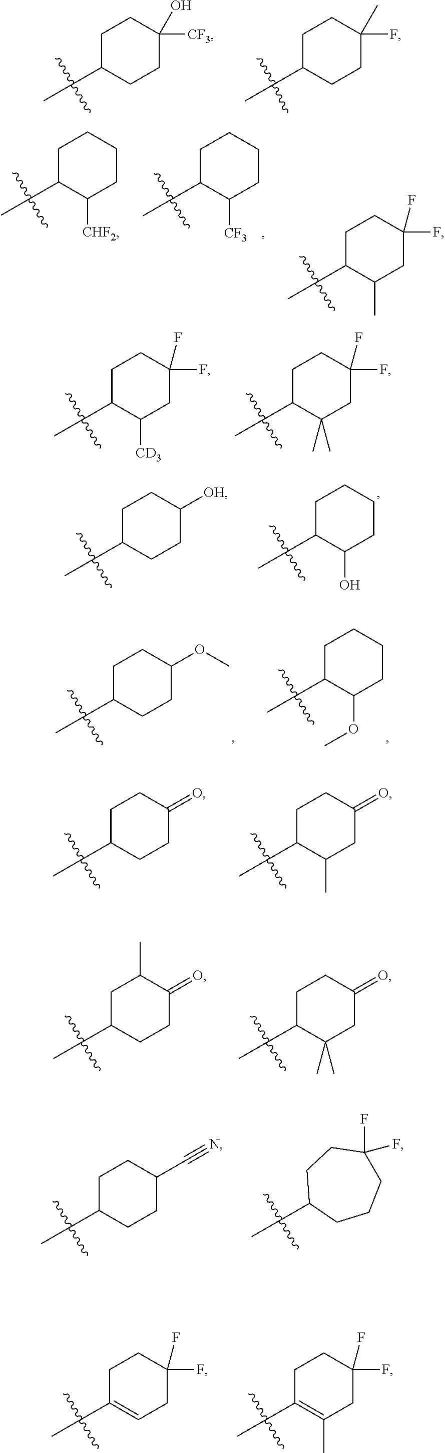

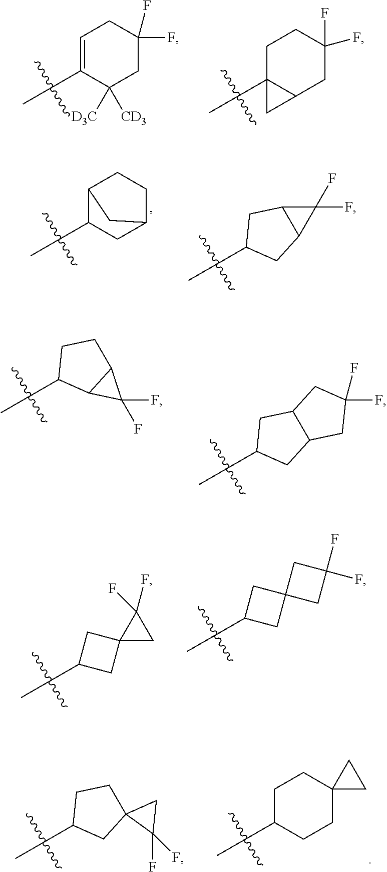

In some embodiments, R^{2a} and R^{2b}, together with the carbon to which they are attached, form one of the following structures:

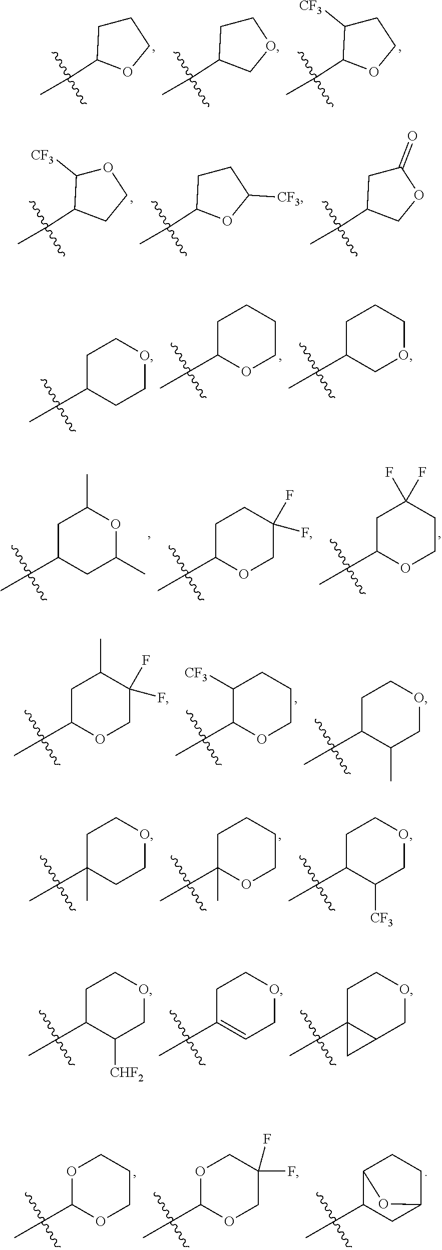

In some embodiments, R^{2a} and R^{2b}, together with the carbon to which they are attached, form one of the following structures:

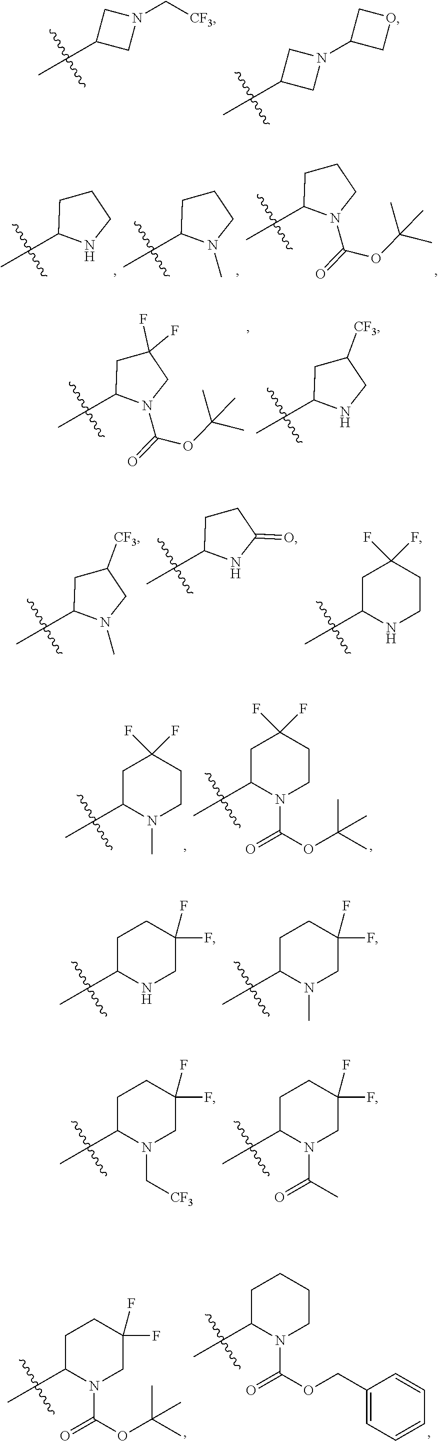

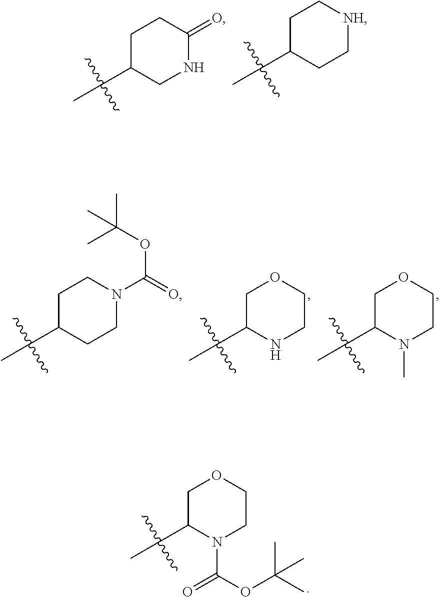

In some embodiments, R^{2a} and R^{2b}, together with the carbon to which they are attached, form one of the following structures:

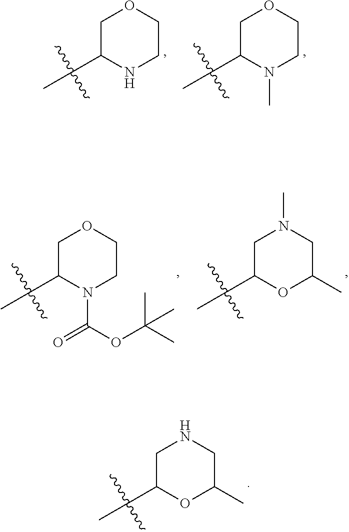

In certain embodiments, R^{2a} and R^{2b}, together with the carbon to which they are attached, form one of the following structures:

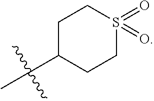 [SMILES: *C1CCS(=O)(=O)CC1]

In some embodiments, R^{2a} and R^{2b}, together with the carbon to which they are attached, form one of the following structures:

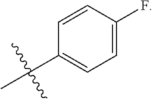 [SMILES: *c1ccc(F)cc1]

In certain embodiments, R^{2a} and R^{2b}, together with the carbon to which they are attached, form one of the following structures:

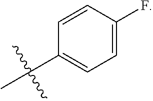 [SMILES: *c1ccc(F)cc1]

In some specific embodiments, R³ has one of the following structures:

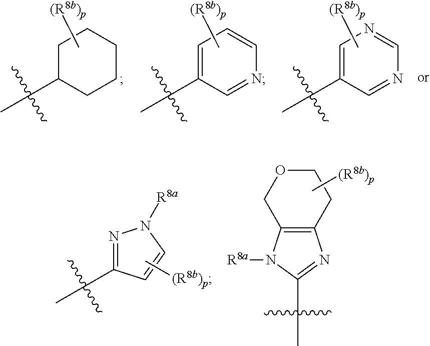

as a stereoisomer, enantiomer, or tautomer thereof or a mixture thereof; or a pharmaceutically acceptable salt, solvate, or prodrug thereof.

In some embodiments, R³ is alkyl. In certain embodiments, R³ is cyanoalkyl. In some embodiments, R³ is —R⁵OR⁶. In certain embodiments, R³ is —R⁵N(R⁶)₂. In some embodiments, R³ is an optionally substituted cycloalkyl. In some embodiments, R³ is an optionally substituted aryl. In certain other embodiments, R³ is an optionally substituted heterocyclyl. In more specific embodiments, R³ is an optionally substituted N-heterocyclyl. In some embodiments, R³ is an optionally substituted heteroaryl. In some embodiments, R³ is an optionally substituted N-heteroaryl. In certain embodiments, R³ is an optionally substituted 5- or 6-membered heteroaryl. In some embodiments, R³ is an optionally substituted fused bicyclic heteroaryl. In some embodiments, R³ is an optionally substituted cycloalkylalkyl. In certain embodiments, R³ is an optionally substituted heterocyclylalkyl.

In some embodiments, R³ has one of the following structures:

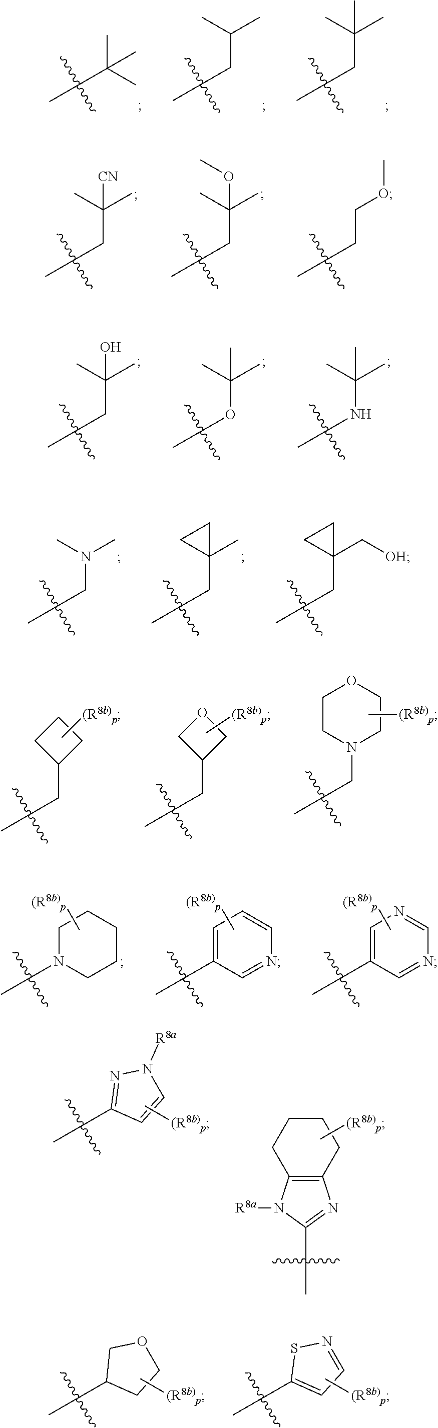

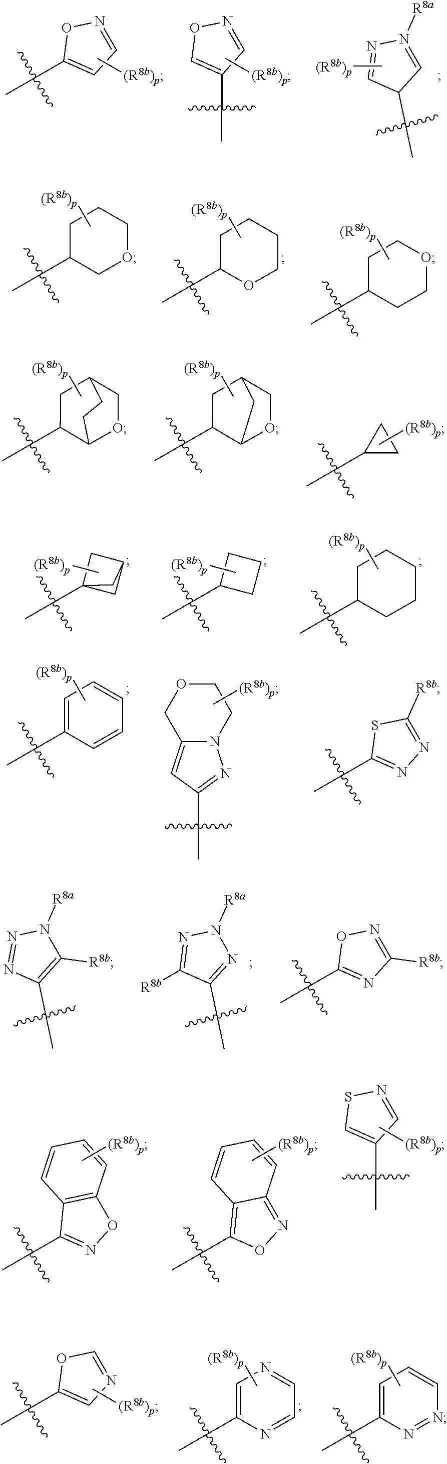

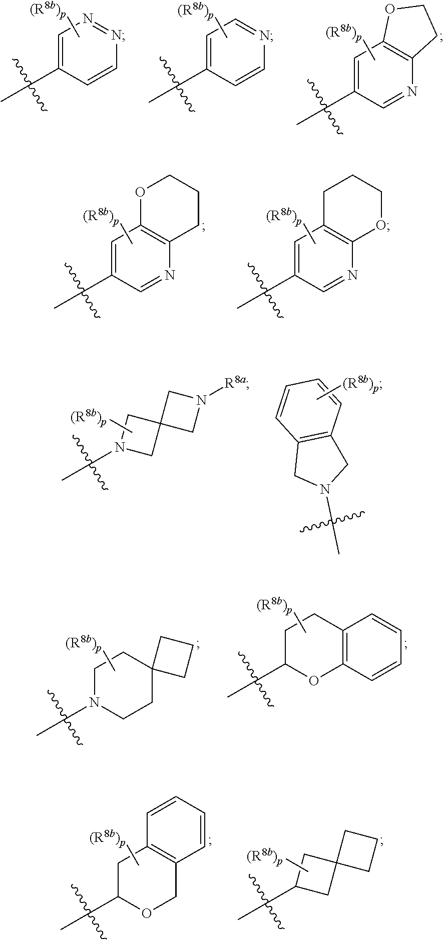

wherein:
R^{8a} is hydrogen, alkyl, haloalkyl, —C(═O)OR^{8d}, optionally substituted aryl, optionally substituted heterocyclylalkyl, optionally substituted cycloalkylalkyl, or optionally substituted cycloalkyl;
each R^{8b} is independently alkyl, optionally substituted cycloalkyl, cyano, halo, —R^{8c}OR^{8d}, —OR^{8c}N(R^{8d})₂, —C(═O)N(R^{8d})₂, —R^{8c}N(R^{8d})₂, or optionally substituted heterocyclyl,
each R^{8c} is independently a direct bond or an optionally substituted alkylene chain;
each R^{8d} is independently hydrogen, alkyl, haloalkyl, hydroxyalkyl, alkoxyalkyl, haloalkoxyalkyl, optionally substituted heterocyclyl, optionally substituted heterocyclylalkyl, optionally substituted cycloalkyl, or optionally substituted cycloalkylalkyl;
or two R_{8d}'s, together with the nitrogen to which they are both attached, form an optionally substituted heterocyclyl;
p is 0, 1, 2, 3, 4, or 5; and

In certain embodiments, R³ has one of the following structures:

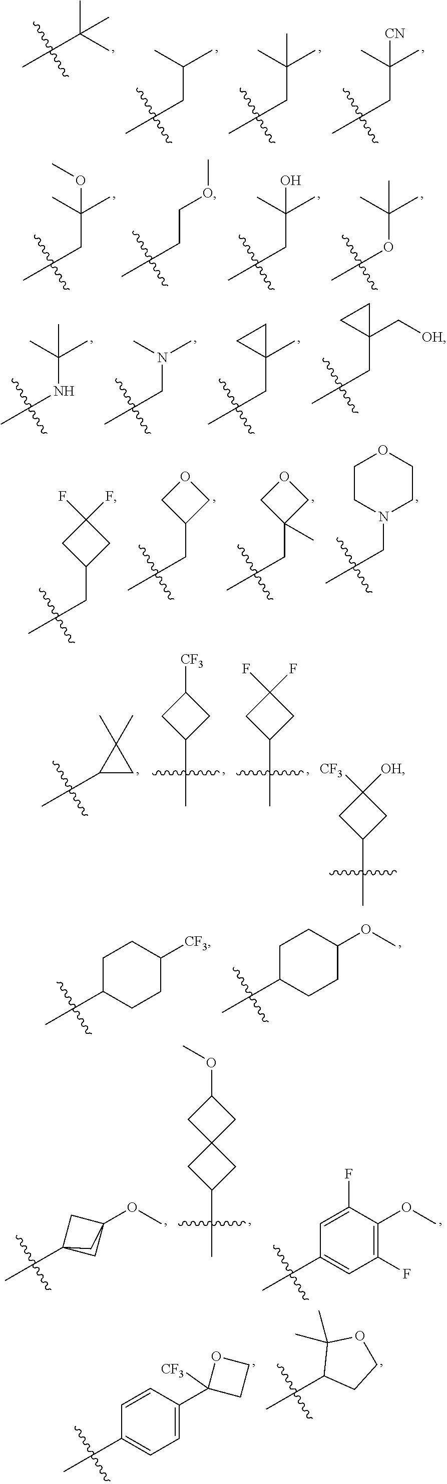

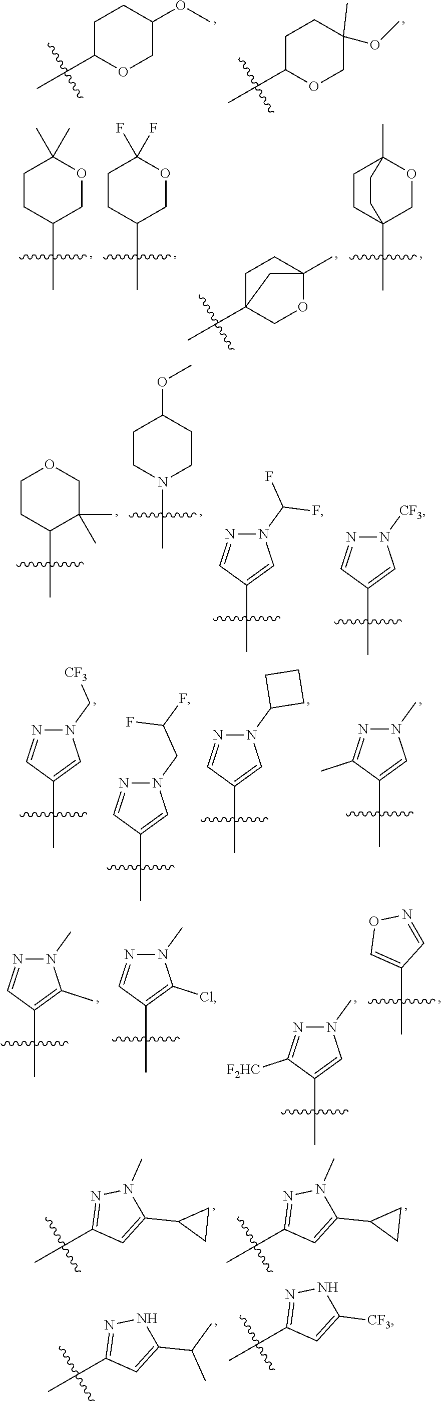

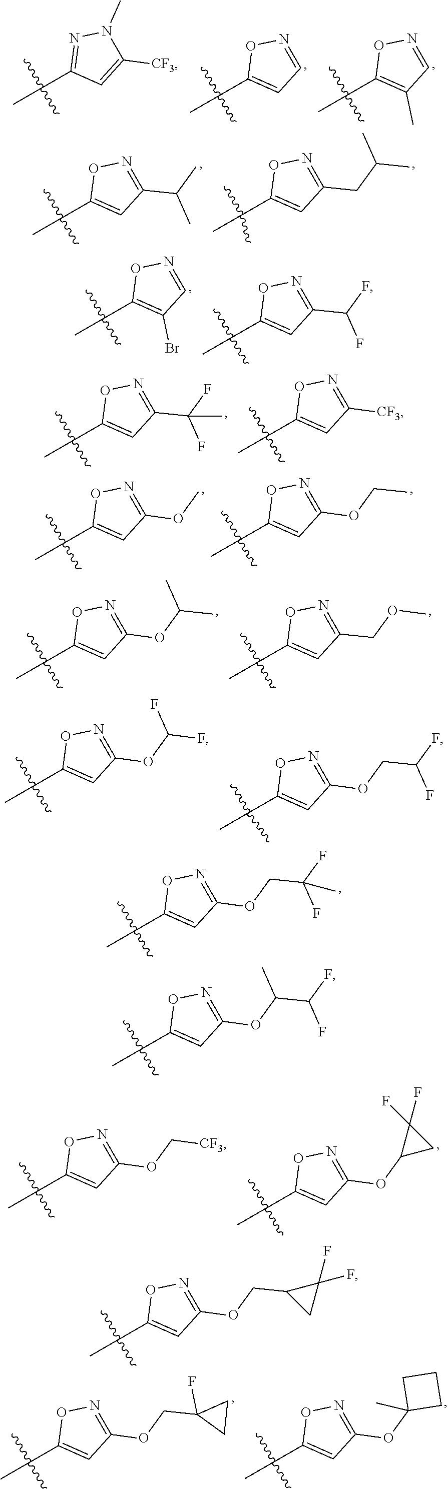

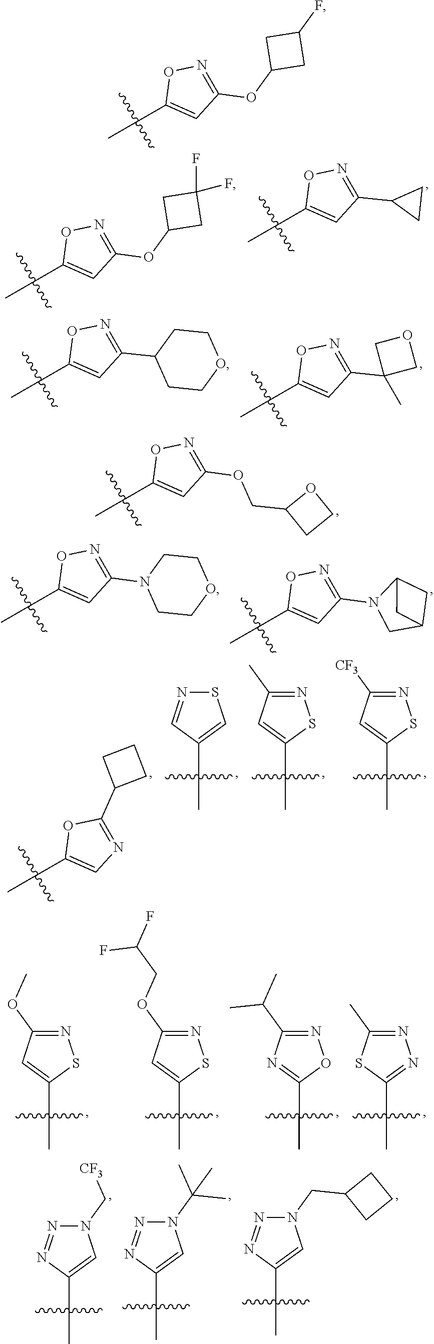

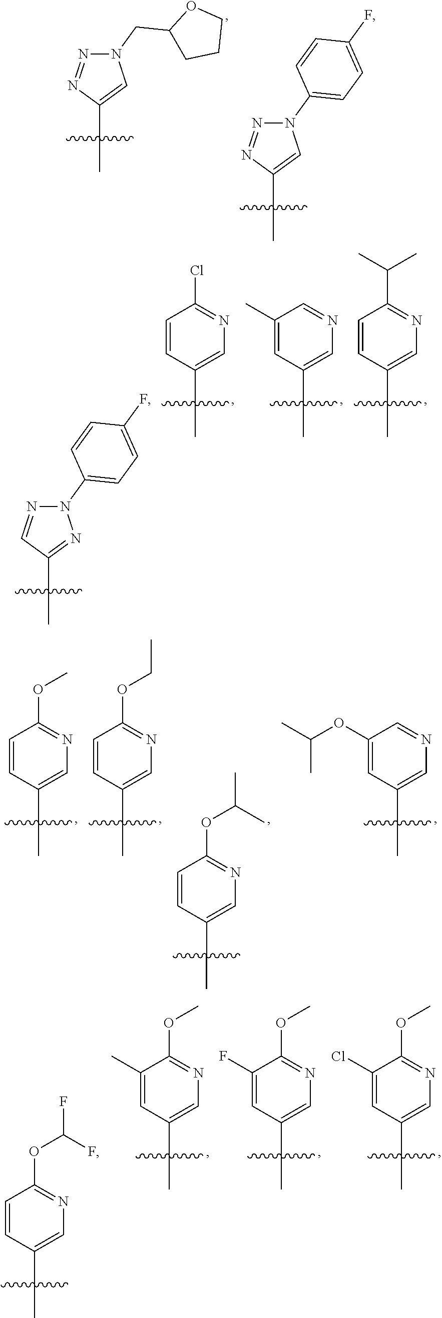

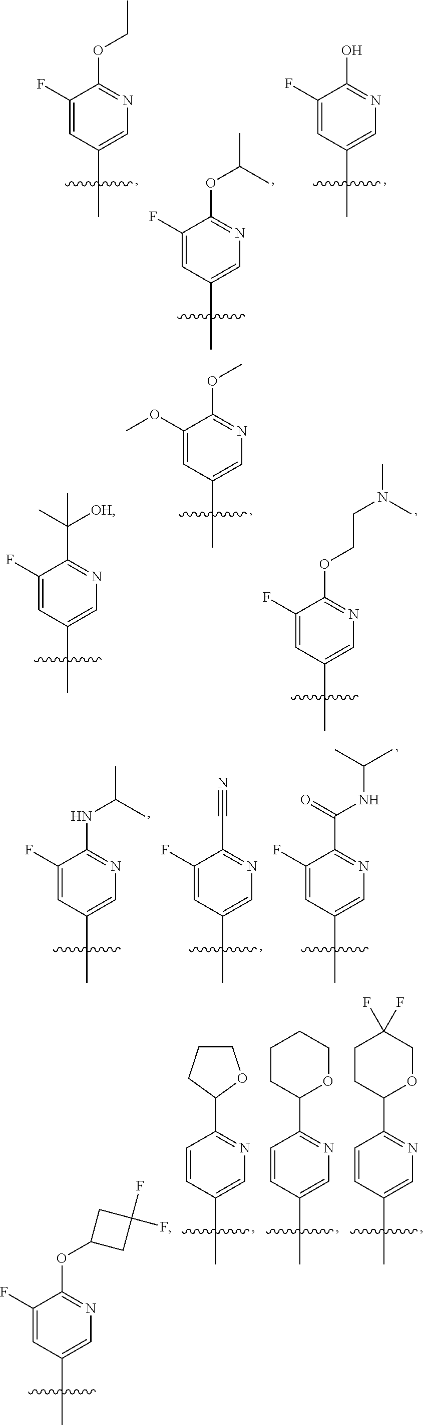

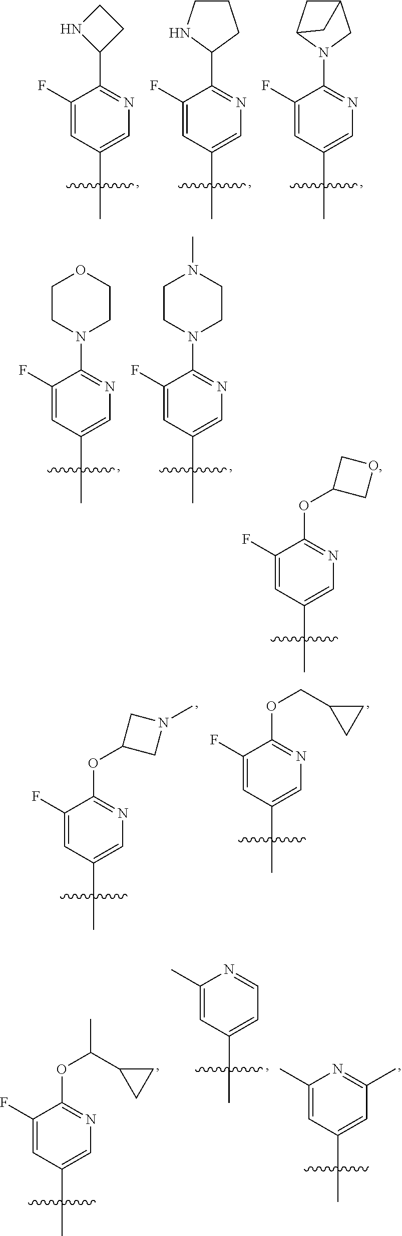

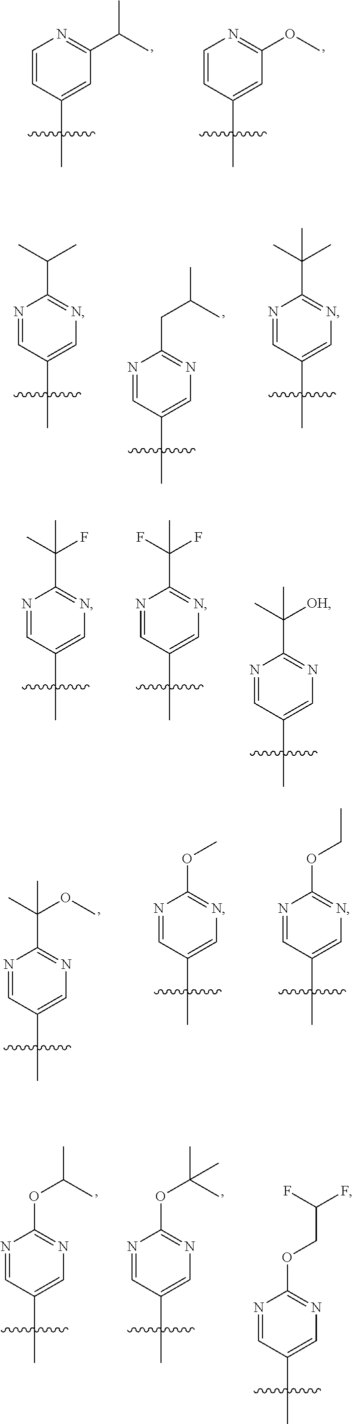

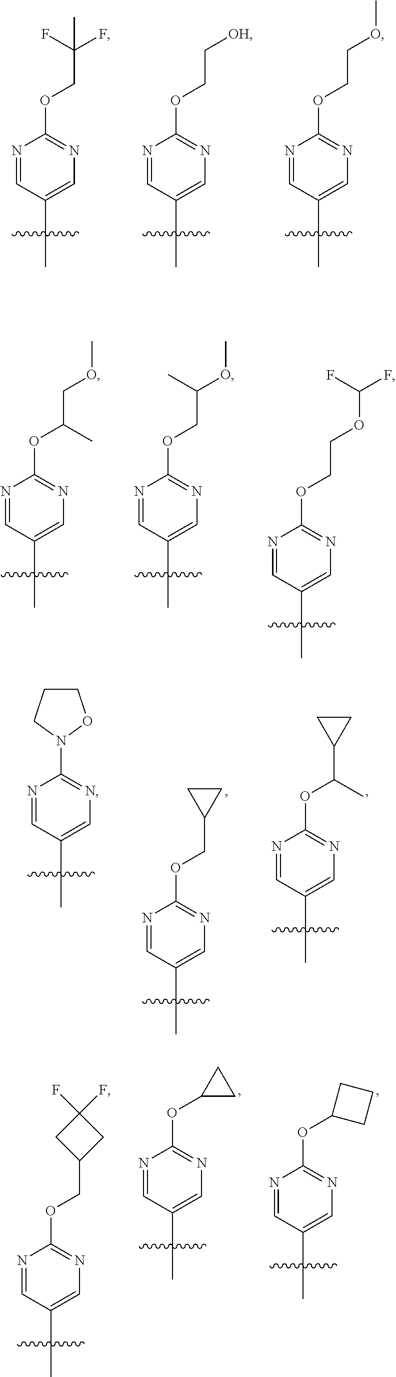

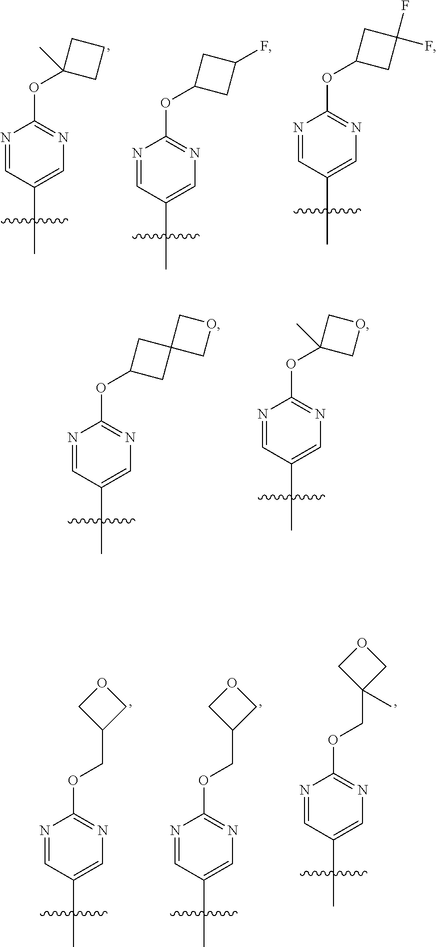

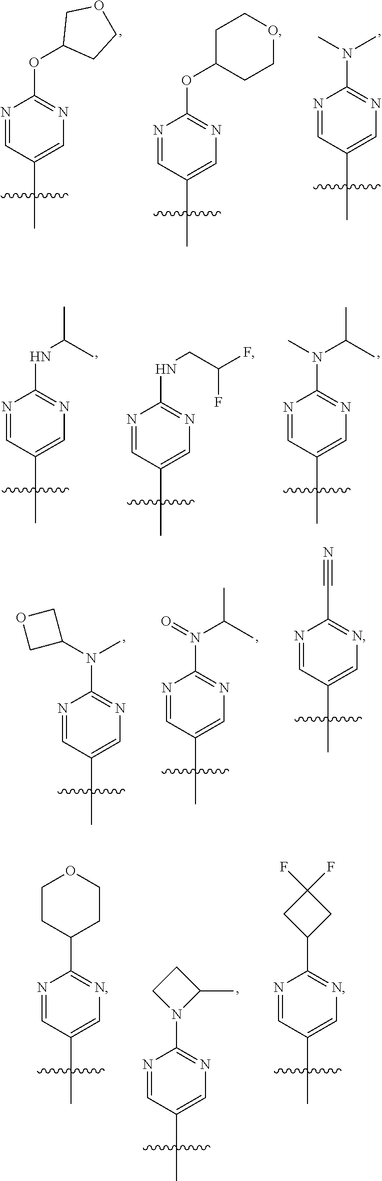

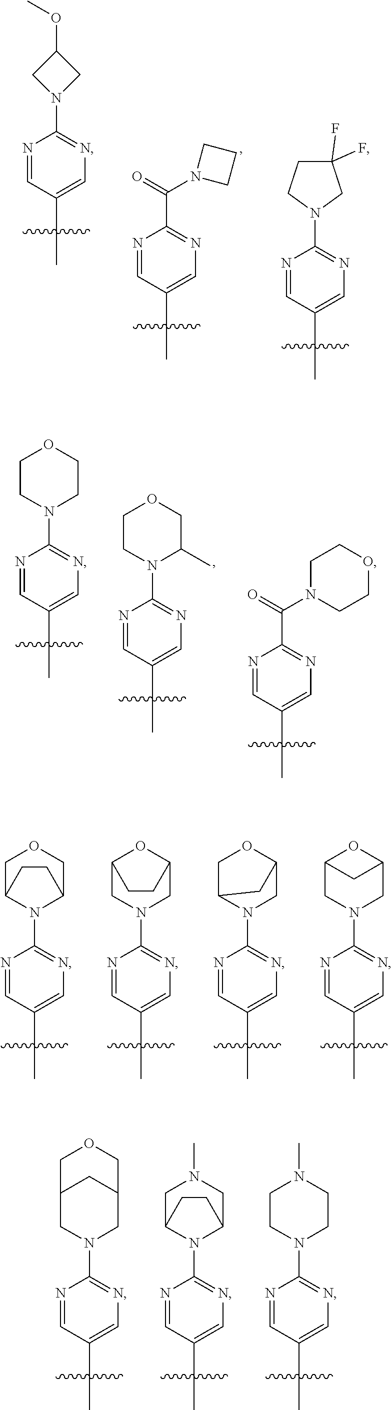

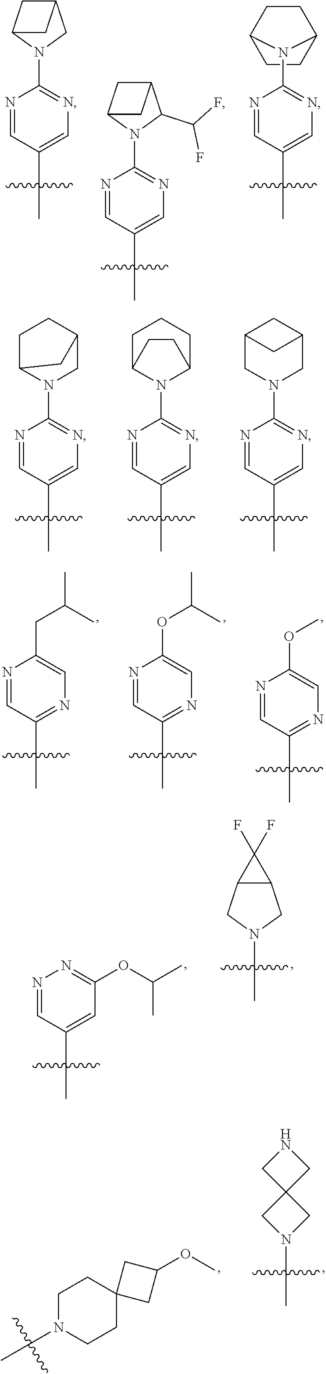

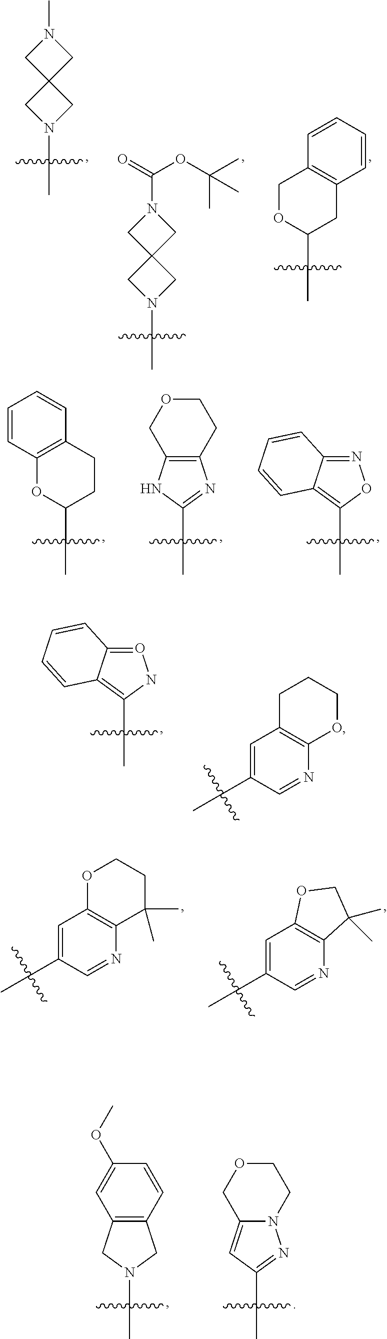

In some embodiments, R³ has one of the following structures:

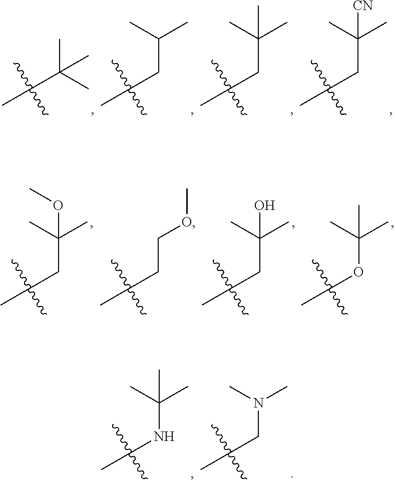

In certain embodiments, R³ has one of the following structures:

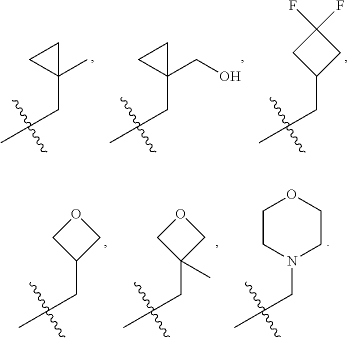

In some embodiments, R³ has one of the following structures:

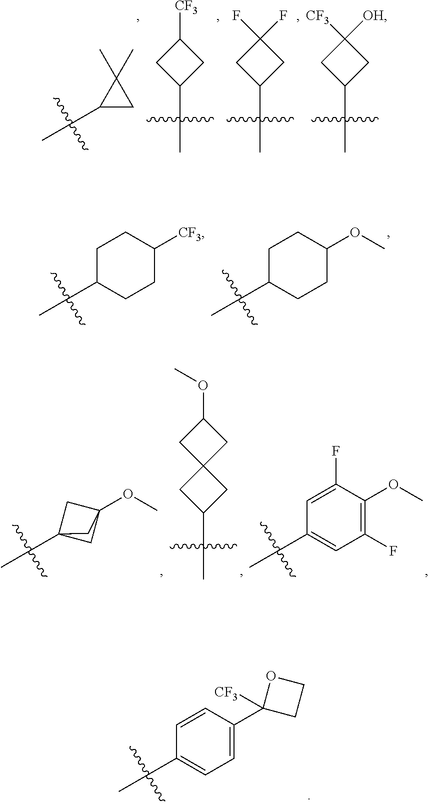

In certain embodiments, R³ has one of the following structures:

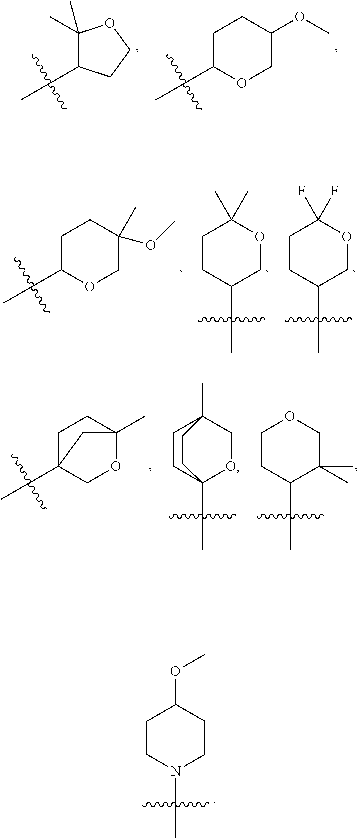

In some embodiments, R³ has one of the following structures:

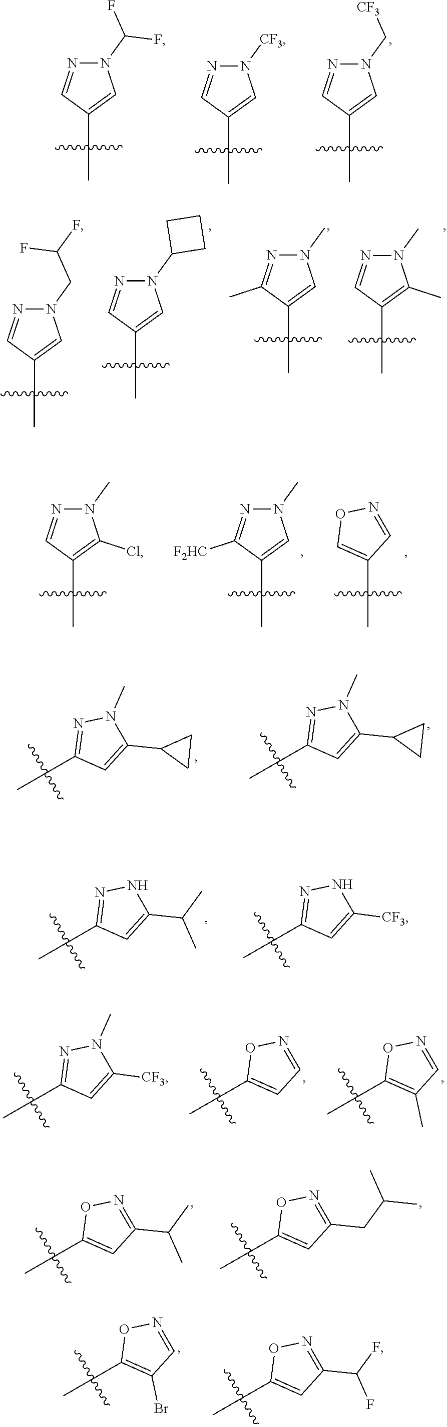

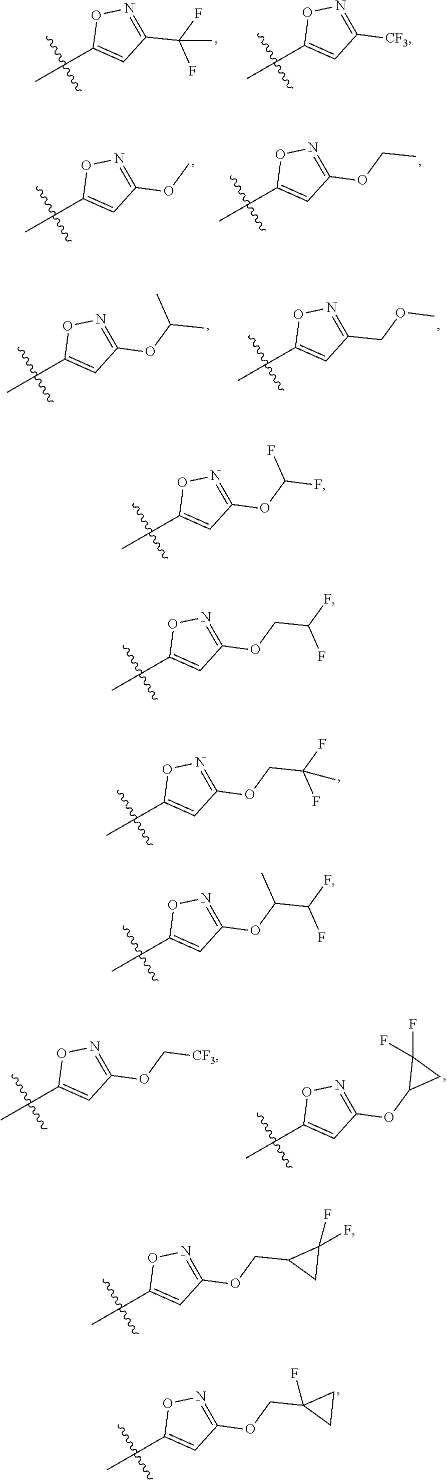

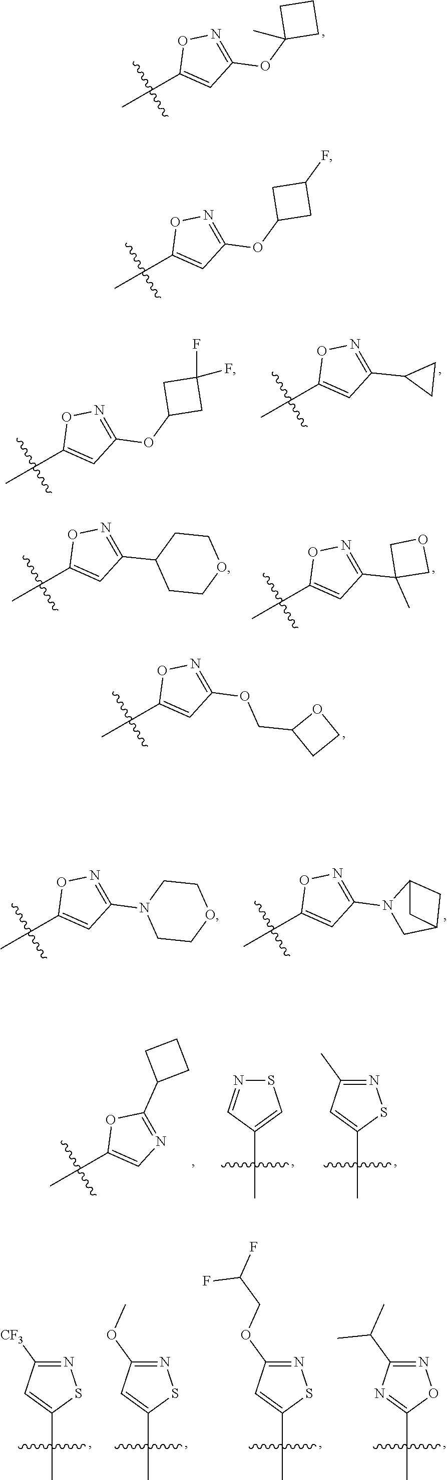

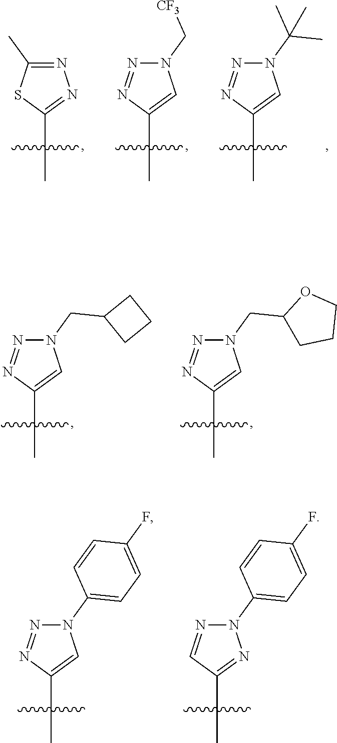

In some embodiments, R³ has one of the following structures:

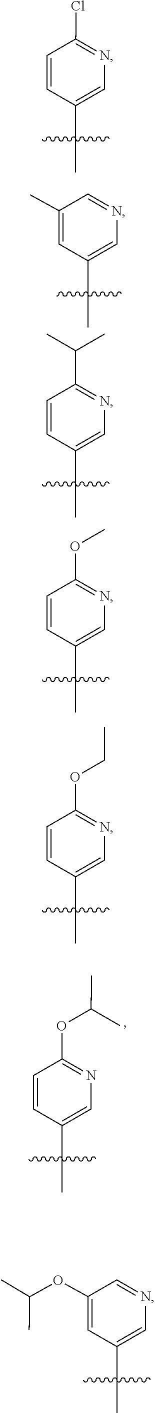

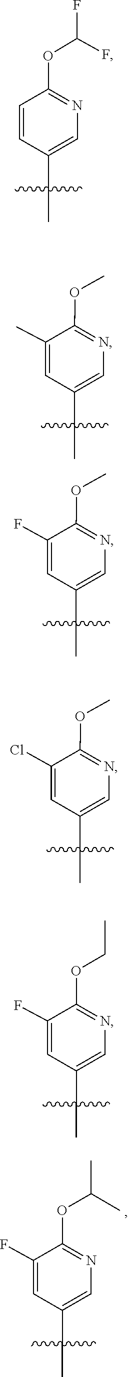

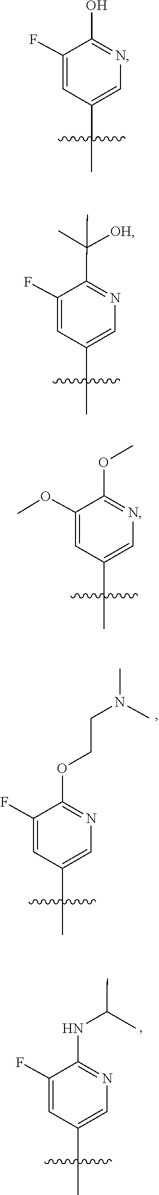 [SMILES: *c1cnc(O)c(F)c1 ; *c1cnc(C(C)(C)O)c(F)c1 ; *c1cnc(OC)c(OC)c1 ; *c1cnc(OCCN(C)C)c(F)c1 ; *c1cnc(NC(C)C)c(F)c1]

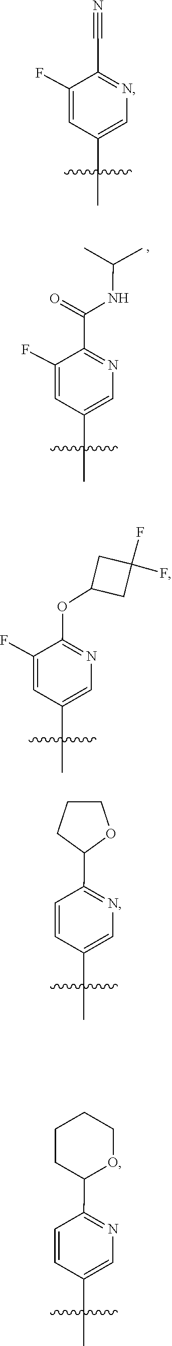 [SMILES: *c1cnc(C#N)c(F)c1 ; *c1cnc(C(=O)NC(C)C)c(F)c1 ; *c1cnc(OC2CC(F)(F)C2)c(F)c1 ; *c1ccc(C2CCCO2)nc1 ; *c1ccc(C2CCCCO2)nc1]

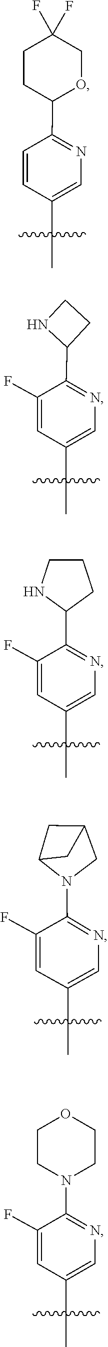 [SMILES: *c1ccc(C2CCC(F)(F)CO2)nc1 ; *c1cnc(C2CCN2)c(F)c1 ; *c1cnc(C2CCCN2)c(F)c1 ; *c1cnc(N2CC3CC2C3)c(F)c1 ; *c1cnc(N2CCOCC2)c(F)c1]

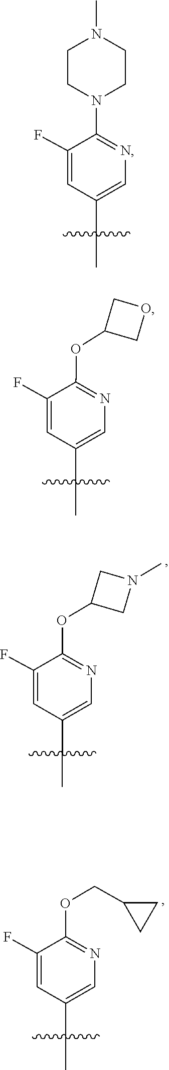 [SMILES: *c1cnc(N2CCN(C)CC2)c(F)c1 ; *c1cnc(OC2COC2)c(F)c1 ; *c1cnc(OC2CN(C)C2)c(F)c1 ; *c1cnc(OCC2CC2)c(F)c1]

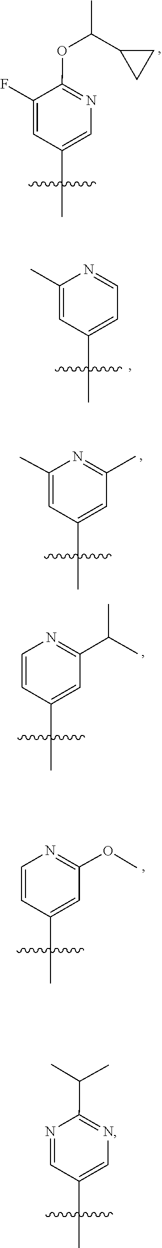

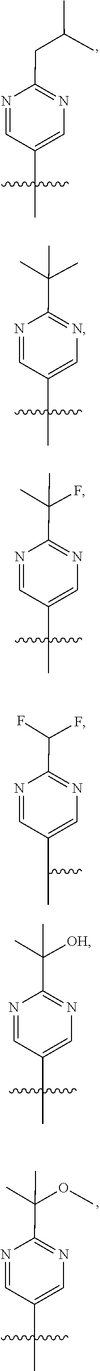

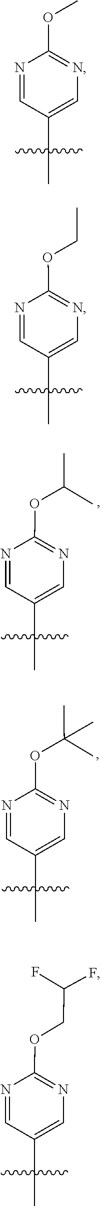

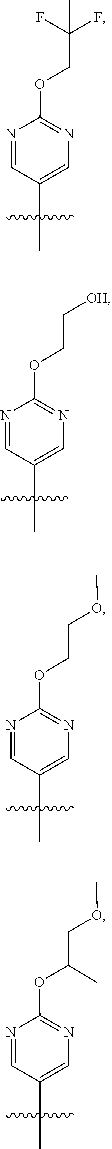 [SMILES: *c1cnc(OCC(C)(F)F)nc1 ; *c1cnc(OCCO)nc1 ; *c1cnc(OCCOC)nc1 ; *c1cnc(OC(C)COC)nc1]

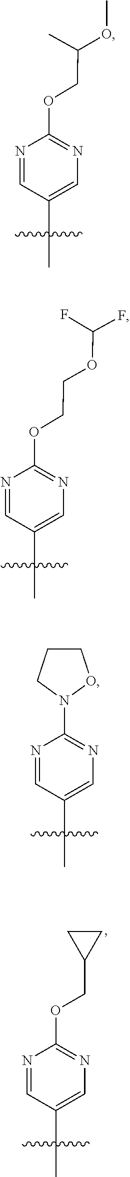 [SMILES: *c1cnc(OCC(C)OC)nc1 ; *c1cnc(OCCOC(F)F)nc1 ; *c1cnc(N2CCCO2)nc1 ; *c1cnc(OCC2CC2)nc1]

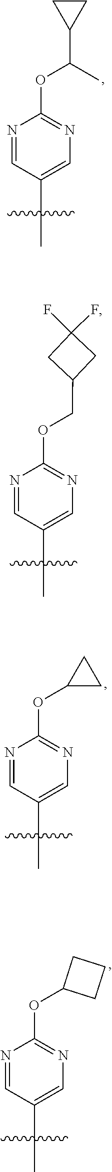 [SMILES: *c1cnc(OC(C)C2CC2)nc1 ; *c1cnc(OCC2CC(F)(F)C2)nc1 ; *c1cnc(OC2CC2)nc1 ; *c1cnc(OC2CCC2)nc1]

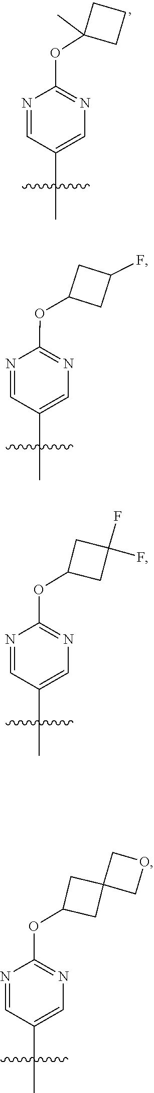 [SMILES: *c1cnc(OC2(C)CCC2)nc1 ; *c1cnc(OC2CC(F)C2)nc1 ; *c1cnc(OC2CC(F)(F)C2)nc1 ; *c1cnc(OC2CC3(COC3)C2)nc1]

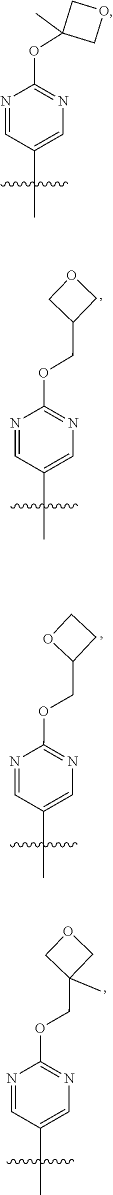 [SMILES: *c1cnc(OC2(C)COC2)nc1 ; *c1cnc(OCC2COC2)nc1 ; *c1cnc(OCC2CCO2)nc1 ; *c1cnc(OCC2(C)COC2)nc1]

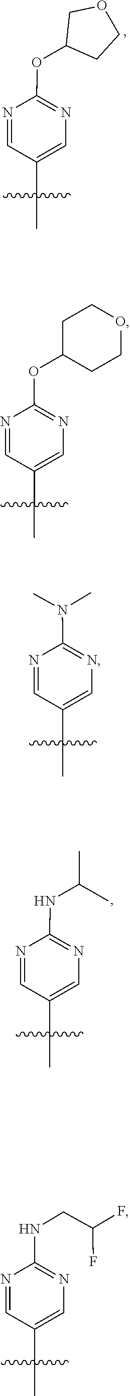

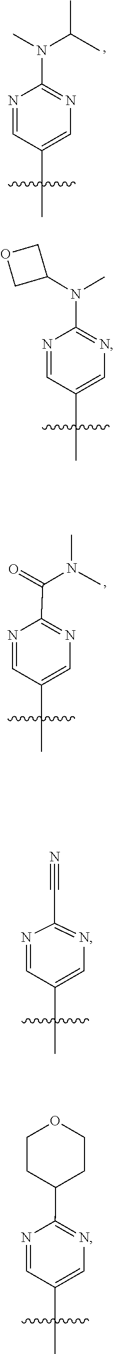 [SMILES: *c1cnc(N(C)C(C)C)nc1 ; *c1cnc(N(C)C2COC2)nc1 ; *c1cnc(C(=O)N(C)C)nc1 ; *c1cnc(C#N)nc1 ; *c1cnc(C2CCOCC2)nc1]

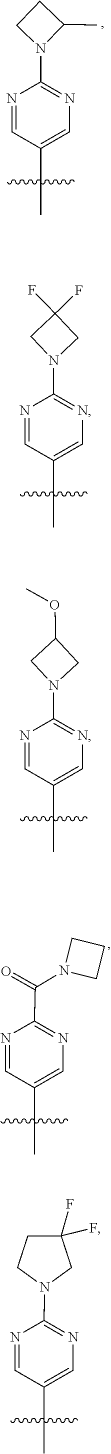 [SMILES: *c1cnc(N2CCC2C)nc1 ; *c1cnc(N2CC(F)(F)C2)nc1 ; *c1cnc(N2CC(OC)C2)nc1 ; *c1cnc(C(=O)N2CCC2)nc1 ; *c1cnc(N2CCC(F)(F)C2)nc1]

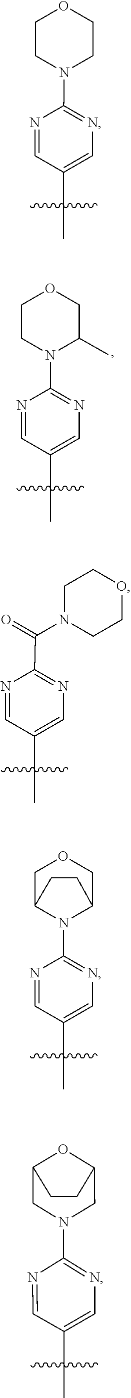 [SMILES: *c1cnc(N2CCOCC2)nc1 ; *c1cnc(N2CCOCC2C)nc1 ; *c1cnc(C(=O)N2CCOCC2)nc1 ; *c1cnc(N2C3CCC2COC3)nc1 ; *c1cnc(N2CC3CCC(C2)O3)nc1]

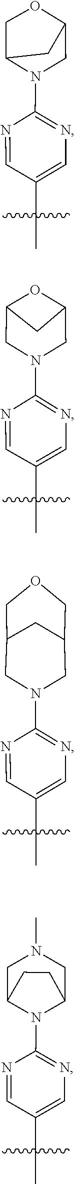 [SMILES: *c1cnc(N2CC3CC2CO3)nc1 ; *c1cnc(N2CC3CC(C2)O3)nc1 ; *c1cnc(N2CC3COCC(C3)C2)nc1 ; *c1cnc(N2C3CCC2CN(C)C3)nc1]

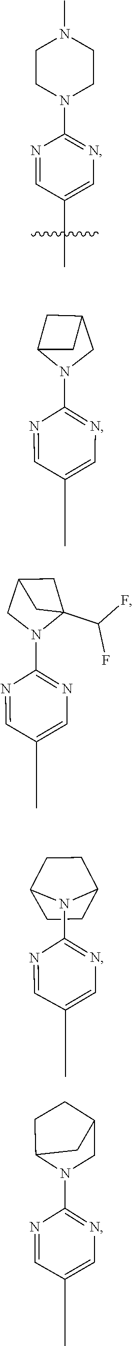

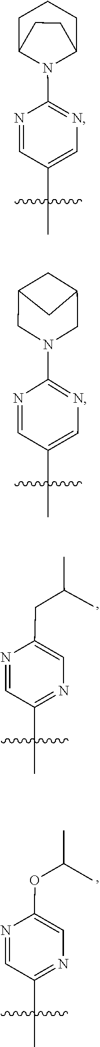 [SMILES: *c1cnc(N2C3CCCC2CC3)nc1 ; *c1cnc(N2CC3CC(C3)C2)nc1 ; *c1cnc(CC(C)C)cn1 ; *c1cnc(OC(C)C)cn1]

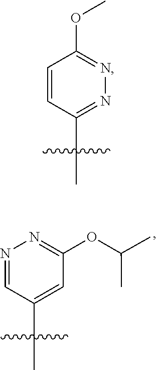

In certain embodiments, R³ has one of the following structures:

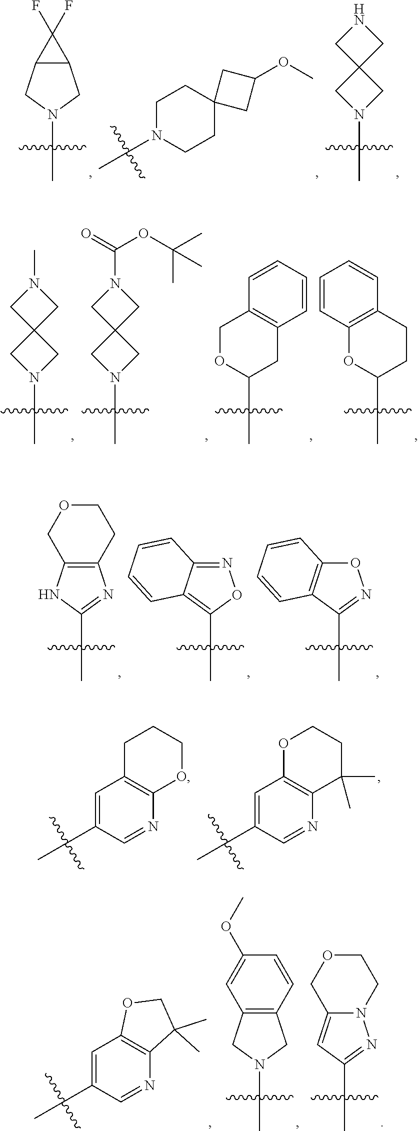

In certain embodiments, L is a direct bond. In some embodiments, L is —C(═O)NR⁴— (i.e., L is —C(═O)NR⁴—* where * indicates a connection to R³). In some embodiments, L is —NR⁴C(═O)— (i.e., L is —NR⁴C(═O)—* where * indicates a connection to R³).

In some embodiments, R⁴ is hydrogen. In some embodiments, R⁴ is methyl. In some embodiments, R⁴ is ethyl, propyl, iso-propyl, butyl, isobutyl, or sec-butyl.

In certain embodiments,

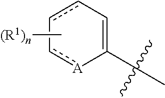

has one of the following structures:

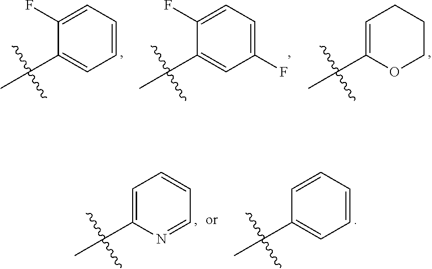 [SMILES: *c1ccccc1F ; *c1cc(F)ccc1F ; *C1=CCCCO1 ; *c1ccccn1 ; *c1ccccc1]

In more specific embodiments,

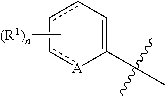

has the following structure:

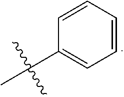 [SMILES: *c1ccccc1]

In some specific embodiments,

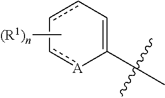

has the following structure:

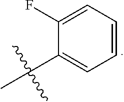 [SMILES: *c1ccccc1F]

In certain embodiments,

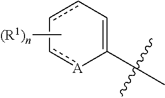

has the following structure:

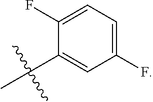 [SMILES: *c1cc(F)ccc1F]

In some more specific embodiments,

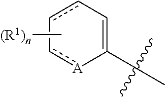

has the following structure:

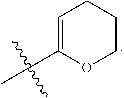 [SMILES: *C1=CCCCO1]

In certain embodiments,

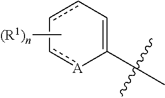

has the following structure:

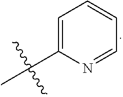 [SMILES: *c1ccccn1]

In certain more specific embodiments,

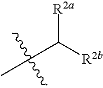

has one of the following structures:

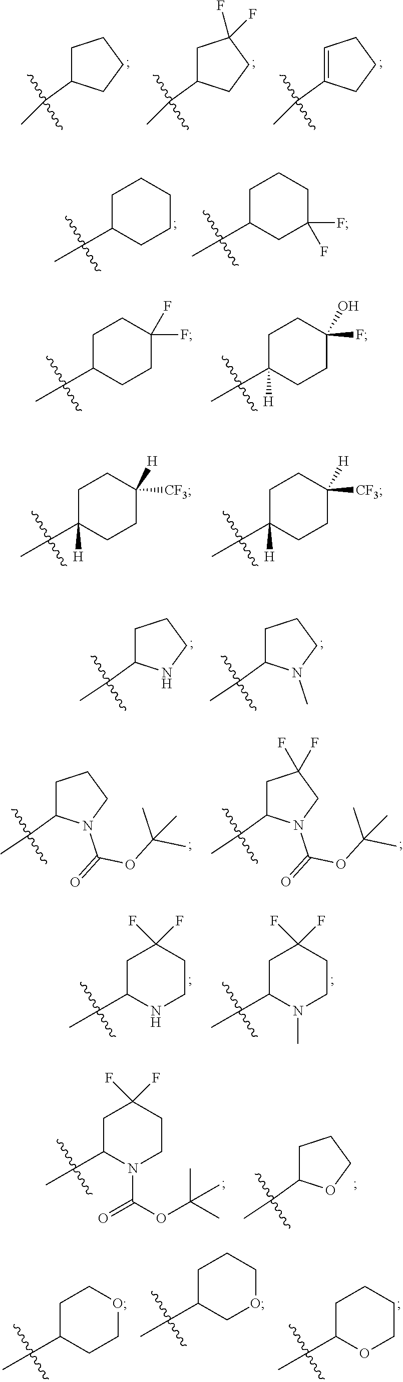

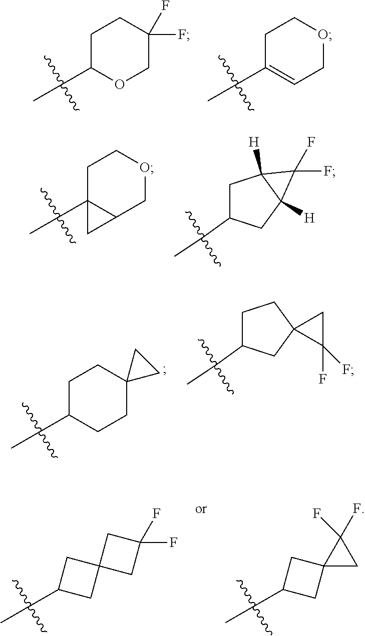

In some embodiments,

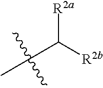

has one of the following structures:

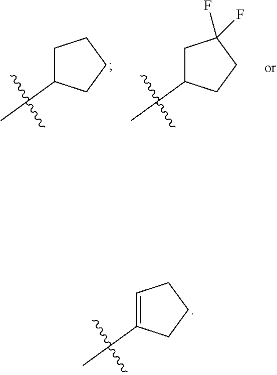 [SMILES: *C1CCCC1 ; *C1CCC(F)(F)C1 ; *C1=CCCC1]

In some embodiments,

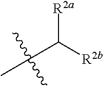

has one of the following structures:

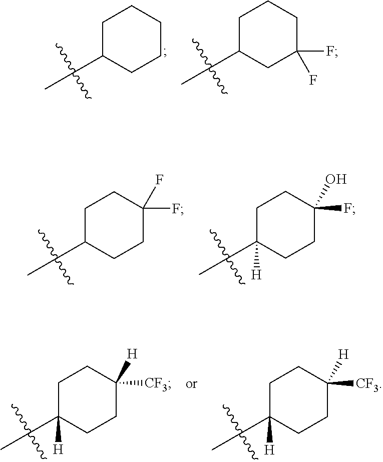 [SMILES: *C1CCCCC1 ; *C1CCCC(F)(F)C1 ; *C1CCC(F)(F)CC1 ; *[C@H]1CC[C@@](O)(F)CC1 ; *[C@H]1CC[C@@H](C(F)(F)F)CC1 ; *[C@H]1CC[C@H](C(F)(F)F)CC1]

In certain embodiments,

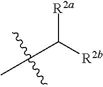

has one o the following structures:

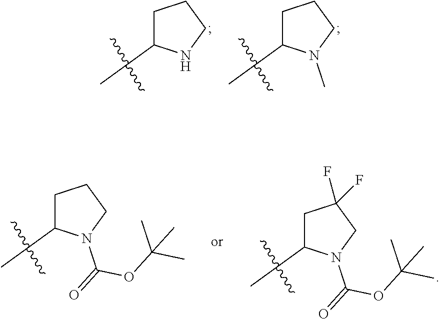

In certain embodiments,

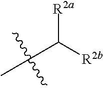

has one of the following structures:

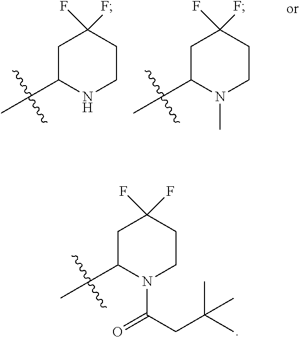

In some embodiments,

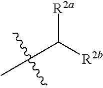

has the following structure:

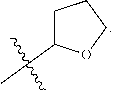 [SMILES: *C1CCCO1]

In some specific embodiments,

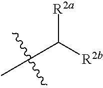

has one of the following structures:

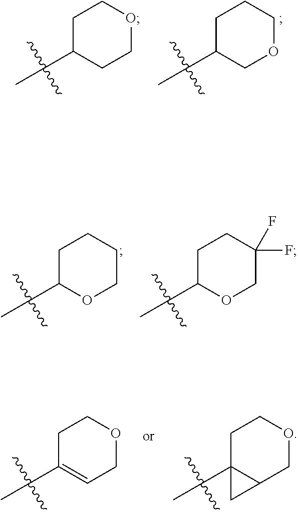 [SMILES: *C1CCOCC1 ; *C1CCCOC1 ; *C1CCCCO1 ; *C1CCC(F)(F)CO1 ; *C1=CCOCC1 ; *C12CCOCC1C2]

In some embodiments,

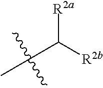

has one of the following structures:

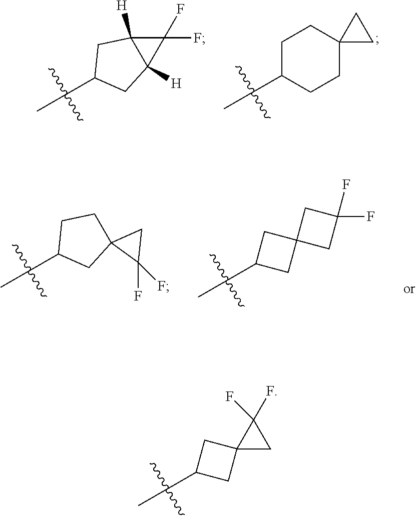 [SMILES: *C1C[C@@H]2[C@H](C1)C2(F)F ; *C1CCC2(CC1)CC2 ; *C1CCC2(C1)CC2(F)F ; *C1CC2(C1)CC(F)(F)C2 ; *C1CC2(C1)CC2(F)F]

In more specific embodiments, -L-R³ has one of the following structures:

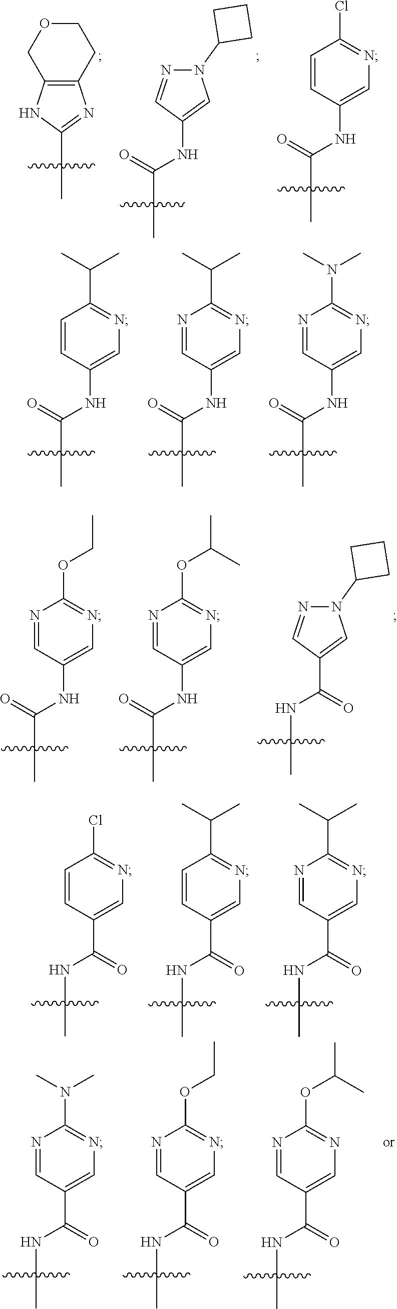

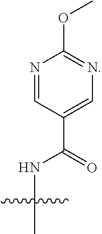 [SMILES: *NC(=O)c1cnc(OC)nc1]

In some embodiments, -L-R³ has the following structure:

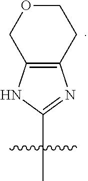 [SMILES: *c1nc2c([nH]1)COCC2]

In more specific embodiments, -L-R³ has one of the following structures:

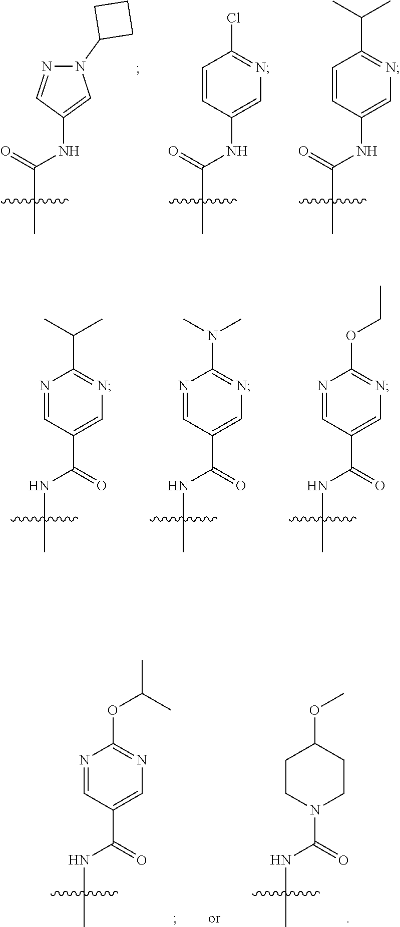

In certain embodiments, -L-R³ has the following structure:

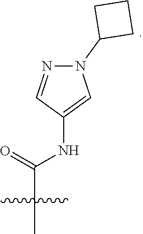 [SMILES: *C(=O)Nc1cnn(C2CCC2)c1]

In some specific embodiments, -L-R³ has one of the following structures:

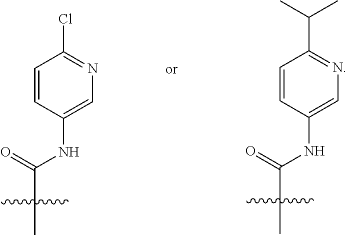 [SMILES: *C(=O)Nc1ccc(Cl)nc1 ; *C(=O)Nc1ccc(C(C)C)nc1]

In some embodiments, -L-R³ has one of the following structures:

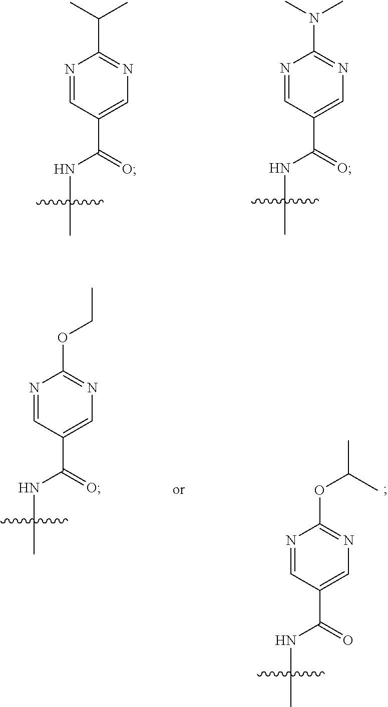 [SMILES: *NC(=O)c1cnc(C(C)C)nc1 ; *NC(=O)c1cnc(N(C)C)nc1 ; *NC(=O)c1cnc(OCC)nc1 ; *NC(=O)c1cnc(OC(C)C)nc1]

In certain more specific embodiments, -L-R³ has the following structure:

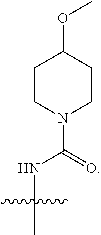 [SMILES: *NC(=O)N1CCC(OC)CC1]

In some embodiments, the compound is a compound as set forth in Table 1 below as a stereoisomer, enantiomer, or tautomer thereof or mixtures thereof; or a pharmaceutically acceptable salt, solvate, or prodrug thereof.

TABLE 1
Representative compounds of formula (I) or (II)

| Number | Compound Structure | Compound Name |
|---|---|---|
| 1 | 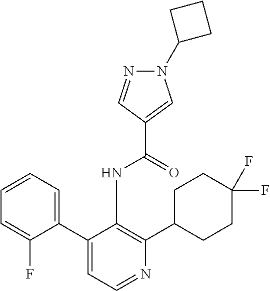 [SMILES: O=C(Nc1c(-c2ccccc2F)ccnc1C1CCC(F)(F)CC1)c1cnn(C2CCC2)c1] | 1-cyclobutyl-N-[2-(4,4- difluorocyclohexyl)-4-(2- fluorophenyl)-3-pyridyl]pyrazole- 4-carboxamide |
| 2 | 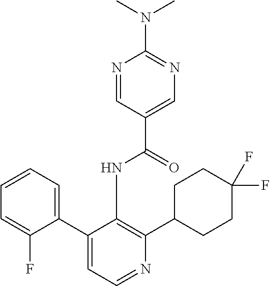 [SMILES: CN(C)c1ncc(C(=O)Nc2c(-c3ccccc3F)ccnc2C2CCC(F)(F)CC2)cn1] | N-[2-(4,4-difluorocyclohexyl)-4- (2-fluorophenyl)-3-pyridyl]-2- (dimethylamino)pyrimidine-5- carboxamide |
| 3 | 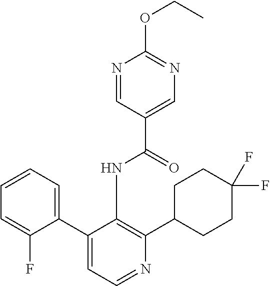 [SMILES: CCOc1ncc(C(=O)Nc2c(-c3ccccc3F)ccnc2C2CCC(F)(F)CC2)cn1] | N-[2-(4,4-difluorocyclohexyl)-4- (2-fluorophenyl)-3-pyridyl]-2- ethoxy-pyrimidine-5- carboxamide |
| 4 | 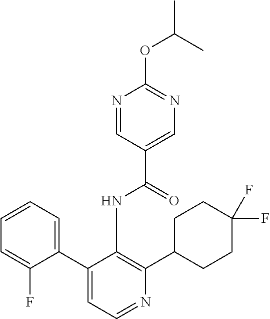 [SMILES: CC(C)Oc1ncc(C(=O)Nc2c(-c3ccccc3F)ccnc2C2CCC(F)(F)CC2)cn1] | N-[2-(4,4-difluorocyclohexyl)-4- (2-fluorophenyl)-3-pyridyl]-2- isopropoxy-pyrimidine-5- carboxamide |
| 5 | 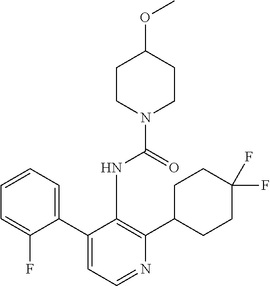 [SMILES: COC1CCN(C(=O)Nc2c(-c3ccccc3F)ccnc2C2CCC(F)(F)CC2)CC1] | N-[2-(4,4-difluorocyclohexyl)-4- (2-fluorophenyl)-3-pyridyl]-4- methoxy-piperidine-1- carboxamide |
| 6 | 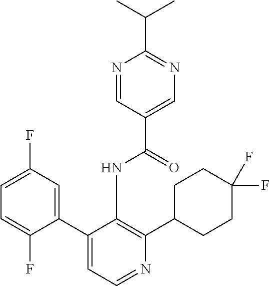 [SMILES: CC(C)c1ncc(C(=O)Nc2c(-c3cc(F)ccc3F)ccnc2C2CCC(F)(F)CC2)cn1] | N-[2-(4,4-difluorocyclohexyl)-4- (2,5-difluorophenyl)-3-pyridyl]-2- isopropyl-pyrimidine-5- carboxamide |
| 7 | 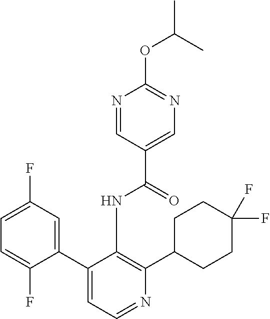 [SMILES: CC(C)Oc1ncc(C(=O)Nc2c(-c3cc(F)ccc3F)ccnc2C2CCC(F)(F)CC2)cn1] | N-[2-(4,4-difluorocyclohexyl)-4- (2,5-difluorophenyl)-3-pyridyl]-2- isopropoxy-pyrimidine-5- carboxamide |
| 8 | 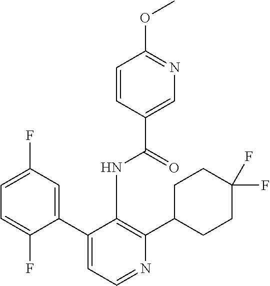 [SMILES: COc1ccc(C(=O)Nc2c(-c3cc(F)ccc3F)ccnc2C2CCC(F)(F)CC2)cn1] | N-[2-(4,4-difluorocyclohexyl)-4- (2,5-difluorophenyl)-3-pyridyl]-6- methoxy-pyridine-3-carboxamide |
| 9 | 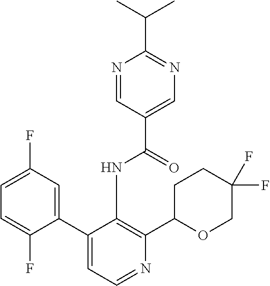 [SMILES: CC(C)c1ncc(C(=O)Nc2c(-c3cc(F)ccc3F)ccnc2C2CCC(F)(F)CO2)cn1] | N-[4-(2,5-difluorophenyl)-2-(5,5- difluorotetrahydropyran-2-yl)-3- pyridyl]-2-isopropyl-pyrimidine-5- carboxamide |
| 10 | 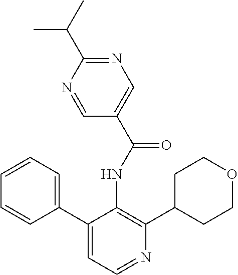 [SMILES: CC(C)c1ncc(C(=O)Nc2c(-c3ccccc3)ccnc2C2CCOCC2)cn1] | 2-isopropyl-N-(4-phenyl-2- tetrahydropyran-4-yl-3- pyridyl)pyrimidine-5- carboxamide |
| 11 | 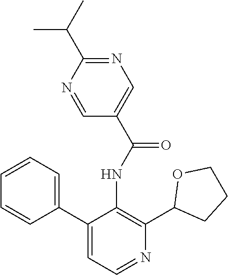 [SMILES: CC(C)c1ncc(C(=O)Nc2c(-c3ccccc3)ccnc2C2CCCO2)cn1] | 2-isopropyl-N-(4-phenyl-2- tetrahydrofuran-2-yl-3- pyridyl)pyrimidine-5- carboxamide |
| 12 | 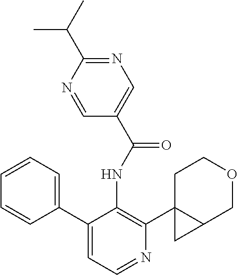 [SMILES: CC(C)c1ncc(C(=O)Nc2c(-c3ccccc3)ccnc2C23CCOCC2C3)cn1] | 2-isopropyl-N-[2-(3- oxabicyclo[4.1.0]heptan-6-yl)-4- phenyl-3-pyridyl]pyrimidine-5- carboxamide |
| 13 | 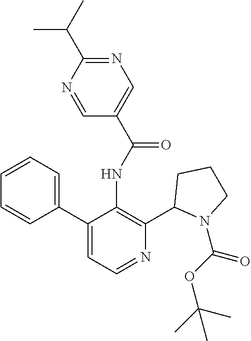 [SMILES: CC(C)c1ncc(C(=O)Nc2c(-c3ccccc3)ccnc2C2CCCN2C(=O)OC(C)(C)C)cn1] | tert-butyl 2-[3-[(2- isopropylpyrimidine-5- carbonyl)amino]-4-phenyl-2- pyridyl]pyrrolidine-1-carboxylate |
| 14 | 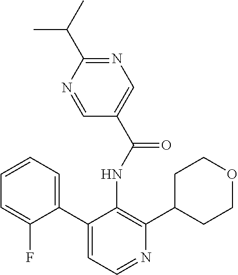 [SMILES: CC(C)c1ncc(C(=O)Nc2c(-c3ccccc3F)ccnc2C2CCOCC2)cn1] | N-[4-(2-fluorophenyl)-2- tetrahydropyran-4-yl-3-pyridyl]-2- isopropyl-pyrimidine-5- carboxamide |
| 15 | 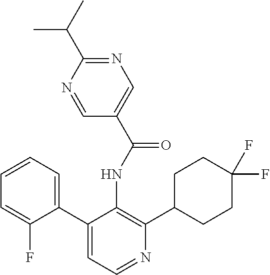 [SMILES: CC(C)c1ncc(C(=O)Nc2c(-c3ccccc3F)ccnc2C2CCC(F)(F)CC2)cn1] | N-[2-(4,4-difluorocyclohexyl)-4- (2-fluorophenyl)-3-pyridyl]-2- isopropyl-pyrimidine-5- carboxamide |
| 16 | 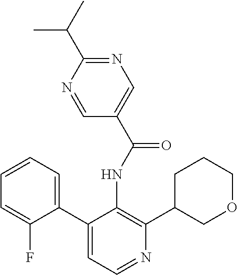 [SMILES: CC(C)c1ncc(C(=O)Nc2c(-c3ccccc3F)ccnc2C2CCCOC2)cn1] | N-[4-(2-fluorophenyl)-2- tetrahydropyran-3-yl-3-pyridyl]-2- isopropyl-pyrimidine-5- carboxamide |
| 17 | 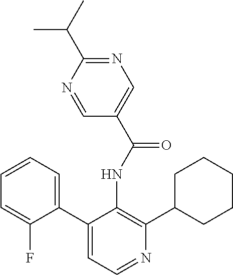 [SMILES: CC(C)c1ncc(C(=O)Nc2c(-c3ccccc3F)ccnc2C2CCCCC2)cn1] | N-[2-cyclohexyl-4-(2- fluorophenyl)-3-pyridyl]-2- isopropyl-pyrimidine-5- carboxamide |
| 18 | 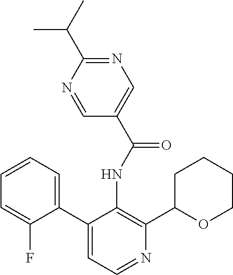 [SMILES: CC(C)c1ncc(C(=O)Nc2c(-c3ccccc3F)ccnc2C2CCCCO2)cn1] | N-[4-(2-fluorophenyl)-2- tetrahydropyran-2-yl-3-pyridyl]-2- isopropyl-pyrimidine-5- carboxamide |
| 19 | 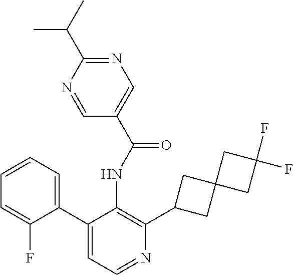 [SMILES: CC(C)c1ncc(C(=O)Nc2c(-c3ccccc3F)ccnc2C2CC3(C2)CC(F)(F)C3)cn1] | N-[2-(2,2- difluorospiro[3.3]heptan-6-yl)-4- (2-fluorophenyl)-3-pyridyl]-2- isopropyl-pyrimidine-5- carboxamide |
| 20 | 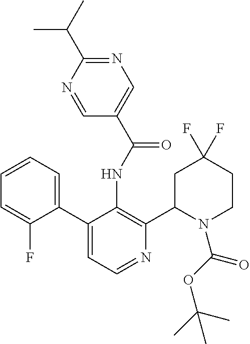 [SMILES: CC(C)c1ncc(C(=O)Nc2c(-c3ccccc3F)ccnc2C2CC(F)(F)CCN2C(=O)OC(C)(C)C)cn1] | tert-butyl 4,4-difluoro-2-[4-(2- fluorophenyl)-3-[(2- isopropylpyrimidine-5- carbonyl)amino]-2- pyridyl]piperidine-1-carboxylate |
| 21 | 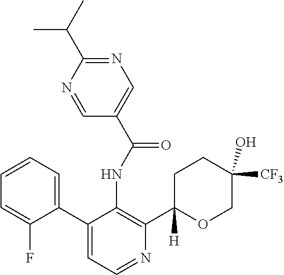 [SMILES: CC(C)c1ncc(C(=O)Nc2c(-c3ccccc3F)ccnc2[C@@H]2CC[C@@](O)(C(F)(F)F)CO2)cn1] | N-[4-(2-fluorophenyl)-2-[4- hydroxy-4- (trifluoromethyl)cyclohexyl]-3- pyridyl]-2-isopropyl-pyrimidine-5- carboxamide |
| 22 | 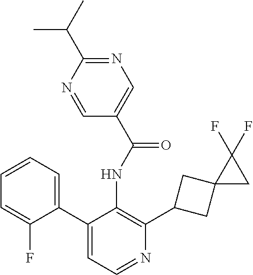 [SMILES: CC(C)c1ncc(C(=O)Nc2c(-c3ccccc3F)ccnc2C2CC3(C2)CC3(F)F)cn1] | N-[2-(2,2- difluorospiro[2.3]hexan-5-yl)-4- (2-fluorophenyl)-3-pyridyl]-2- isopropyl-pyrimidine-5- carboxamide |
| 23 | 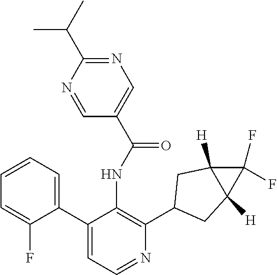 [SMILES: CC(C)c1ncc(C(=O)Nc2c(-c3ccccc3F)ccnc2C2C[C@@H]3[C@H](C2)C3(F)F)cn1] | N-[2-[(1R,5S)-6,6-difluoro-3- bicyclo[3.1.0]hexanyl]-4-(2- fluorophenyl)-3-pyridyl]-2- isopropyl-pyrimidine-5- carboxamide |
| 24 | 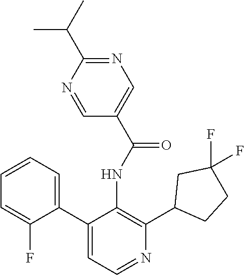 [SMILES: CC(C)c1ncc(C(=O)Nc2c(-c3ccccc3F)ccnc2C2CCC(F)(F)C2)cn1] | N-[2-(3,3-difluorocyclopentyl)-4- (2-fluorophenyl)-3-pyridyl]-2- isopropyl-pyrimidine-5- carboxamide |
| 25 | 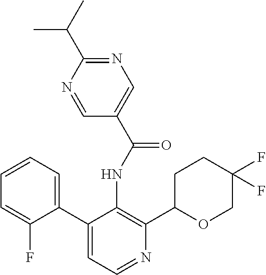 [SMILES: CC(C)c1ncc(C(=O)Nc2c(-c3ccccc3F)ccnc2C2CCC(F)(F)CO2)cn1] | N-[2-(5,5- difluorotetrahydropyran-2-yl)-4- (2-fluorophenyl)-3-pyridyl]-2- isopropyl-pyrimidine-5- carboxamide |
| 26 | 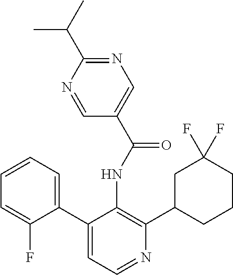 [SMILES: CC(C)c1ncc(C(=O)Nc2c(-c3ccccc3F)ccnc2C2CCCC(F)(F)C2)cn1] | N-[2-(3,3-difluorocyclohexyl)-4- (2-fluorophenyl)-3-pyridyl]-2- isopropyl-pyrimidine-5- carboxamide |
| 27 | 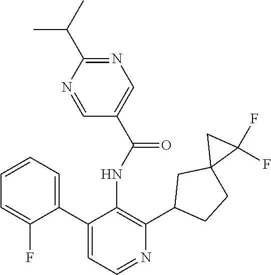 [SMILES: CC(C)c1ncc(C(=O)Nc2c(-c3ccccc3F)ccnc2C2CCC3(C2)CC3(F)F)cn1] | N-[2-(2,2- difluorospiro[2.4]heptan-6-yl)-4- (2-fluorophenyl)-3-pyridyl]-2- isopropyl-pyrimidine-5- carboxamide |
| 28 | 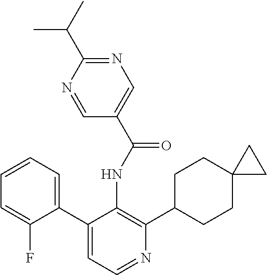 [SMILES: CC(C)c1ncc(C(=O)Nc2c(-c3ccccc3F)ccnc2C2CCC3(CC2)CC3)cn1] | N-[4-(2-fluorophenyl)-2- spiro[2.5]octan-6-yl-3-pyridyl]-2- isopropyl-pyrimidine-5- carboxamide |
| 29 | 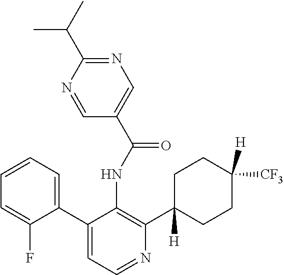 [SMILES: CC(C)c1ncc(C(=O)Nc2c(-c3ccccc3F)ccnc2[C@H]2CC[C@@H](C(F)(F)F)CC2)cn1] | N-[4-(2-fluorophenyl)-2-[4- (trifluoromethyl)cyclohexyl]-3- pyridyl]-2-isopropyl-pyrimidine-5- carboxamide |
| 30 | 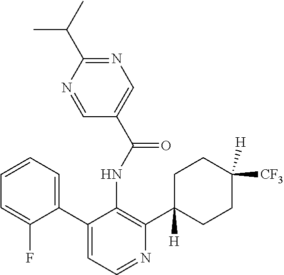 [SMILES: CC(C)c1ncc(C(=O)Nc2c(-c3ccccc3F)ccnc2[C@H]2CC[C@H](C(F)(F)F)CC2)cn1] | N-[4-(2-fluorophenyl)-2-[4- (trifluoromethyl)cyclohexyl]-3- pyridyl]-2-isopropyl-pyrimidine-5- carboxamide |
| 31 | 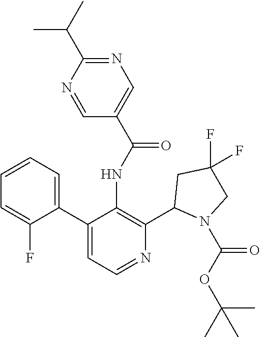 [SMILES: CC(C)c1ncc(C(=O)Nc2c(-c3ccccc3F)ccnc2C2CC(F)(F)CN2C(=O)OC(C)(C)C)cn1] | tert-butyl 4,4-difluoro-2-[4-(2- fluorophenyl)-3-[(2- isopropylpyrimidine-5- carbonyl)amino]-2- pyridyl]pyrrolidine-1-carboxylate |
| 32 | 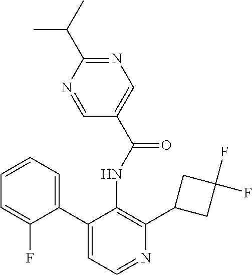 [SMILES: CC(C)c1ncc(C(=O)Nc2c(-c3ccccc3F)ccnc2C2CC(F)(F)C2)cn1] | N-(2-(3,3-difluorocyclobutyl)-4- (2-fluorophenyl)pyridin-3-yl)-2- isopropylpyrimidine-5- carboxamide |
| 33 | 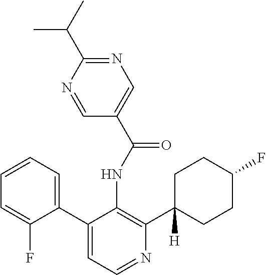 [SMILES: CC(C)c1ncc(C(=O)Nc2c(-c3ccccc3F)ccnc2[C@H]2CC[C@@H](F)CC2)cn1] | N-(2-((1s,4s)-4-fluorocyclohexyl)- 4-(2-fluorophenyl)pyridin-3-yl)-2- isopropylpyrimidine-5- carboxamide |
| 34 | 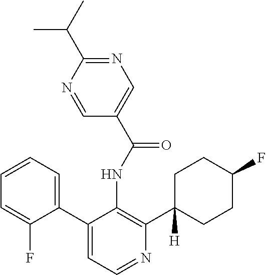 [SMILES: CC(C)c1ncc(C(=O)Nc2c(-c3ccccc3F)ccnc2[C@H]2CC[C@H](F)CC2)cn1] | N-(2-((1r,4r)-4-fluorocyclohexyl)- 4-(2-fluorophenyl)pyridin-3-yl)-2- isopropylpyrimidine-5- carboxamide |
| 35 | 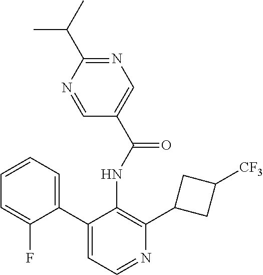 [SMILES: CC(C)c1ncc(C(=O)Nc2c(-c3ccccc3F)ccnc2C2CC(C(F)(F)F)C2)cn1] | N-(4-(2-fluorophenyl)-2-(3- (trifluoromethyl)cyclobutyl)pyridin- 3-yl)-2-isopropylpyrimidine-5- carboxamide |
| 36 | 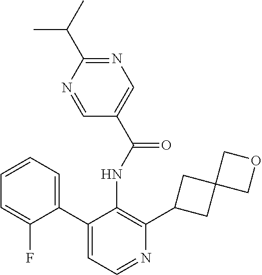 [SMILES: CC(C)c1ncc(C(=O)Nc2c(-c3ccccc3F)ccnc2C2CC3(COC3)C2)cn1] | N-(4-(2-fluorophenyl)-2-(2- oxaspiro[3.3]heptan-6-yl)pyridin- 3-yl)-2-isopropylpyrimidine-5- carboxamide |
| 37 | 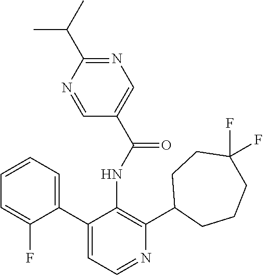 [SMILES: CC(C)c1ncc(C(=O)Nc2c(-c3ccccc3F)ccnc2C2CCCC(F)(F)CC2)cn1] | (±)-N-(2-(4,4- difluorocycloheptyl)-4-(2- fluorophenyl)pyridin-3-yl)-2- isopropylpyrimidine-5- carboxamide |
| 38 | 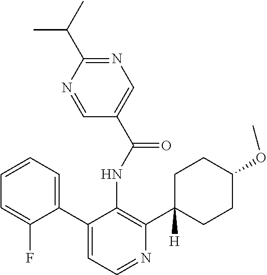 [SMILES: CO[C@H]1CC[C@@H](c2nccc(-c3ccccc3F)c2NC(=O)c2cnc(C(C)C)nc2)CC1] | N-(4-(2-fluorophenyl)-2-((1s,4s)- 4-methoxycyclohexyl)pyridin-3- yl)-2-isopropylpyrimidine-5- carboxamide |
| 39 | 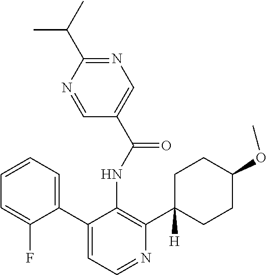 [SMILES: CO[C@H]1CC[C@H](c2nccc(-c3ccccc3F)c2NC(=O)c2cnc(C(C)C)nc2)CC1] | N-(4-(2-fluorophenyl)-2-((1r,4r)- 4-methoxycyclohexyl)pyridin-3- yl)-2-isopropylpyrimidine-5- carboxamide |
| 40 | 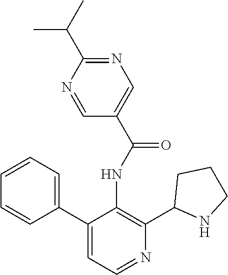 [SMILES: CC(C)c1ncc(C(=O)Nc2c(-c3ccccc3)ccnc2C2CCCN2)cn1] | 2-isopropyl-N-(4-phenyl-2- pyrrolidin-2-yl-3- pyridyl)pyrimidine-5- carboxamide |
| 41 | 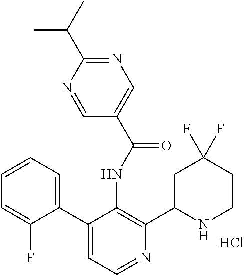 [SMILES: CC(C)c1ncc(C(=O)Nc2c(-c3ccccc3F)ccnc2C2CC(F)(F)CCN2)cn1 ; Cl] | N-[2-(4,4-difluoro-2-piperidyl)-4- (2-fluorophenyl)-3-pyridyl]-2- isopropyl-pyrimidine-5- carboxamide |
| 42 | 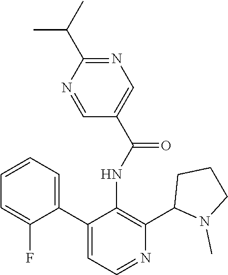 [SMILES: CC(C)c1ncc(C(=O)Nc2c(-c3ccccc3F)ccnc2C2CCCN2C)cn1] | N-[4-(2-fluorophenyl)-2-(1- methylpyrrolidin-2-yl)-3-pyridyl]- 2-isopropyl-pyrimidine-5- carboxamide |
| 43 | 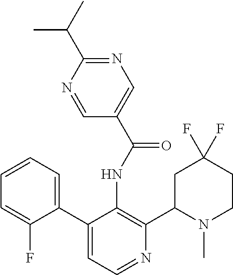 [SMILES: CC(C)c1ncc(C(=O)Nc2c(-c3ccccc3F)ccnc2C2CC(F)(F)CCN2C)cn1] | N-[2-(4,4-difluoro-1-methyl-2- piperidyl)-4-(2-fluorophenyl)-3- pyridyl]-2-isopropyl-pyrimidine-5- carboxamide |
| 44 | 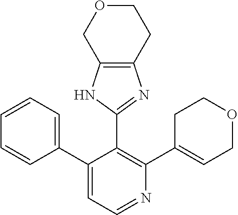 [SMILES: C1=C(c2nccc(-c3ccccc3)c2-c2nc3c([nH]2)COCC3)CCOC1] | 2-(2-(3,6-dihydro-2H-pyran-4-yl)- 4-phenylpyridin-3-yl)-3,4,6,7- tetrahydropyrano[3,4-d]imidazole |
| 45 | 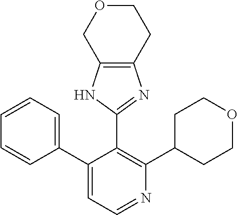 [SMILES: c1ccc(-c2ccnc(C3CCOCC3)c2-c2nc3c([nH]2)COCC3)cc1] | 2-(4-phenyl-2-(tetrahydro-2H- pyran-4-yl)pyridin-3-yl)-3,4,6,7- tetrahydropyrano[3,4-d]imidazole |
| 46 | 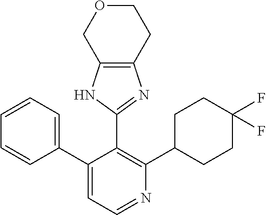 [SMILES: FC1(F)CCC(c2nccc(-c3ccccc3)c2-c2nc3c([nH]2)COCC3)CC1] | 2-(2-(4,4-difluorocyclohexyl)-4- phenylpyridin-3-yl)-3,4,6,7- tetrahydropyrano[3,4-d]imidazole |
| 47 | 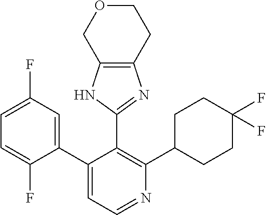 [SMILES: Fc1ccc(F)c(-c2ccnc(C3CCC(F)(F)CC3)c2-c2nc3c([nH]2)COCC3)c1] | 2-(2-(4,4-difluorocyclohexyl)-4- (2,5-difluorophenyl)pyridin-3-yl)- 3,4,6,7-tetrahydropyrano[3,4- d]imidazole |
| 48 | 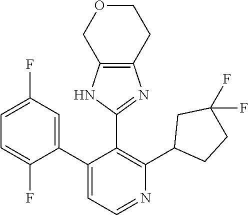 [SMILES: Fc1ccc(F)c(-c2ccnc(C3CCC(F)(F)C3)c2-c2nc3c([nH]2)COCC3)c1] | 2-(2-(3,3-difluorocyclopentyl)-4- (2,5-difluorophenyl)pyridin-3-yl)- 3,4,6,7-tetrahydropyrano[3,4- d]imidazole |
| 49 | 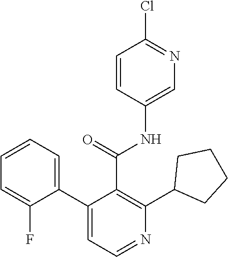 [SMILES: O=C(Nc1ccc(Cl)nc1)c1c(-c2ccccc2F)ccnc1C1CCCC1] | N-(6-chloropyridin-3-yl)-2- cyclopentyl-4-(2- fluorophenyl)nicotinamide |
| 50 | 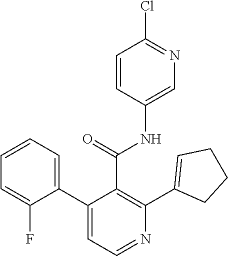 [SMILES: O=C(Nc1ccc(Cl)nc1)c1c(-c2ccccc2F)ccnc1C1=CCCC1] | N-(6-chloropyridin-3-yl)-2- (cyclopent-1-en-1-yl)-4-(2- fluorophenyl)nicotinamide |
| 51 | 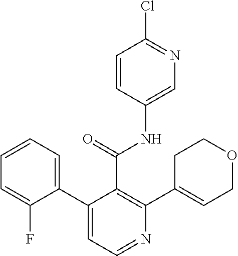 [SMILES: O=C(Nc1ccc(Cl)nc1)c1c(-c2ccccc2F)ccnc1C1=CCOCC1] | N-(6-chloropyridin-3-yl)-2-(3,6- dihydro-2H-pyran-4-yl)-4-(2- fluorophenyl)nicotinamide |
| 52 | 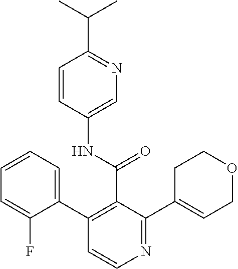 [SMILES: CC(C)c1ccc(NC(=O)c2c(-c3ccccc3F)ccnc2C2=CCOCC2)cn1] | 2-(3,6-dihydro-2H-pyran-4-yl)-4- (2-fluorophenyl)-N-(6- isopropylpyridin-3- yl)nicotinamide |
| 53 | 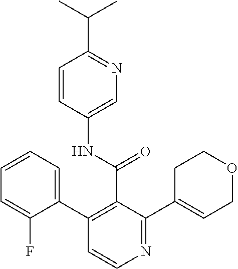 [SMILES: CC(C)c1ccc(NC(=O)c2c(-c3ccccc3F)ccnc2C2=CCOCC2)cn1] | 4-(2-fluorophenyl)-N-(6- isopropylpyridin-3-yl)-2- (tetrahydro-2H-pyran-4- yl)nicotinamide |
| 54 | 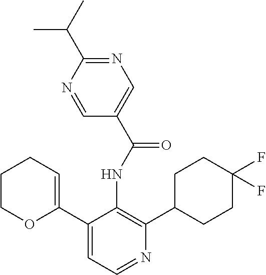 [SMILES: CC(C)c1ncc(C(=O)Nc2c(C3=CCCCO3)ccnc2C2CCC(F)(F)CC2)cn1] | N-(2-(4,4-difluorocyclohexyl)-4- (3,4-dihydro-2H-pyran-6- yl)pyridin-3-yl)-2- isopropylpyrimidine-5- carboxamide |
| 55 | 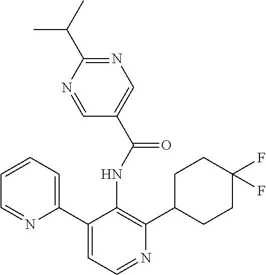 [SMILES: CC(C)c1ncc(C(=O)Nc2c(-c3ccccn3)ccnc2C2CCC(F)(F)CC2)cn1] | N-(2′-(4,4-difluorocyclohexyl)- [2,4′-bipyridin]-3′-yl)-2- isopropylpyrimidine-5- carboxamide |
| 56 | 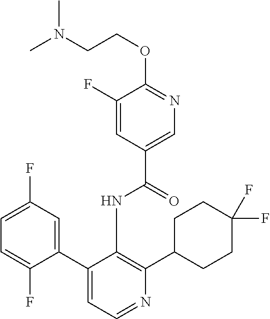 [SMILES: CN(C)CCOc1ncc(C(=O)Nc2c(-c3cc(F)ccc3F)ccnc2C2CCC(F)(F)CC2)cc1F] | N-(2-(4,4-difluorocyclohexyl)-4- (2,5-difluorophenyl)pyridin-3-yl)- 6-(2-(dimethylamino)ethoxy)-5- fluoronicotinamide |
| 57 | 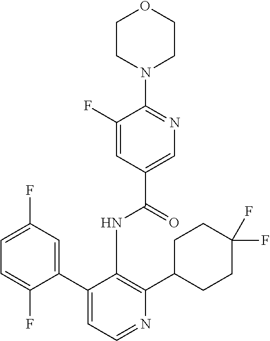 [SMILES: O=C(Nc1c(-c2cc(F)ccc2F)ccnc1C1CCC(F)(F)CC1)c1cnc(N2CCOCC2)c(F)c1] | N-(2-(4,4-difluorocyclohexyl)-4- (2,5-difluorophenyl)pyridin-3-yl)- 5-fluoro-6- morpholinonicotinamide |
| 58 | 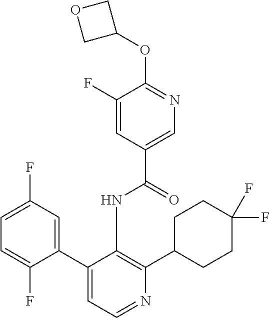 [SMILES: O=C(Nc1c(-c2cc(F)ccc2F)ccnc1C1CCC(F)(F)CC1)c1cnc(OC2COC2)c(F)c1] | N-(2-(4,4-difluorocyclohexyl)-4- (2,5-difluorophenyl)pyridin-3-yl)- 5-fluoro-6-(oxetan-3- yloxy)nicotinamide |
| 59 | 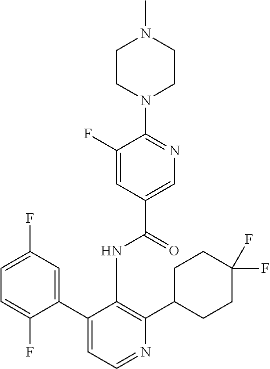 [SMILES: CN1CCN(c2ncc(C(=O)Nc3c(-c4cc(F)ccc4F)ccnc3C3CCC(F)(F)CC3)cc2F)CC1] | N-(2-(4,4-difluorocyclohexyl)-4- (2,5-difluorophenyl)pyridin-3-yl)- 5-fluoro-6-(4-methylpiperazin-1- yl)nicotinamide |
| 60 | 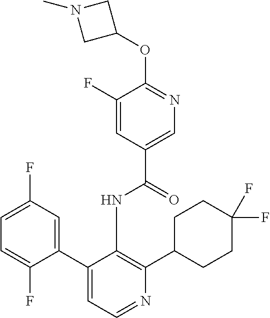 [SMILES: CN1CC(Oc2ncc(C(=O)Nc3c(-c4cc(F)ccc4F)ccnc3C3CCC(F)(F)CC3)cc2F)C1] | N-(2-(4,4-difluorocyclohexyl)-4- (2,5-difluorophenyl)pyridin-3-yl)- 5-fluoro-6-((1-methylazetidin-3- yl)oxy)nicotinamide |
| 61 | 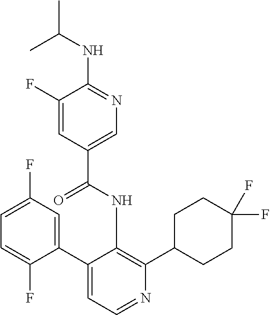 [SMILES: CC(C)Nc1ncc(C(=O)Nc2c(-c3cc(F)ccc3F)ccnc2C2CCC(F)(F)CC2)cc1F] | N-(2-(4,4-difluorocyclohexyl)-4- (2,5-difluoro-phenyl)pyridin-3- yl)-5-fluoro-6- (isopropylamino)nicotinamide |
| 62 | 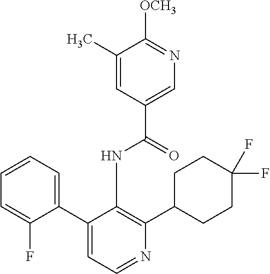 [SMILES: COc1ncc(C(=O)Nc2c(-c3ccccc3F)ccnc2C2CCC(F)(F)CC2)cc1C] | N-(2-(4,4-difluorocyclohexyl)-4- (2-fluorophenyl)pyridin-3-yl)-6- methoxy-5-methylnicotinamide |
| 63 | 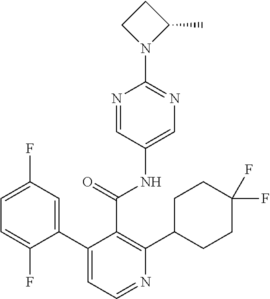 [SMILES: C[C@H]1CCN1c1ncc(NC(=O)c2c(-c3cc(F)ccc3F)ccnc2C2CCC(F)(F)CC2)cn1] | (S)-2-(4,4-difluorocyclohexyl)-4- (2,5-difluorophenyl)-N-(2-(2- methylazetidin-1-yl)pyrimidin-5- yl)nicotinamide |
| 64 | 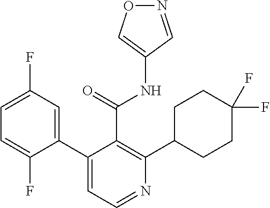 [SMILES: O=C(Nc1cnoc1)c1c(-c2cc(F)ccc2F)ccnc1C1CCC(F)(F)CC1] | 2-(4,4-difluorocyclohexyl)-4- (2,5-difluorophenyl)-N-(isoxazol- 4-yl)nicotinamide |
| 65 | 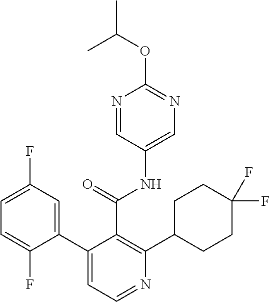 [SMILES: CC(C)Oc1ncc(NC(=O)c2c(-c3cc(F)ccc3F)ccnc2C2CCC(F)(F)CC2)cn1] | 2-(4,4-difluorocyclohexyl)-4-(2,5- difluorophenyl)-N-(2- isopropoxypyrimidin-5- yl)nicotinamide |
| 66 | 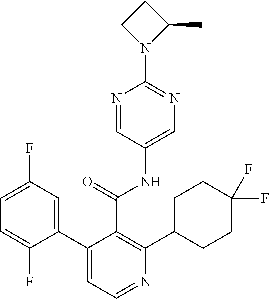 [SMILES: C[C@@H]1CCN1c1ncc(NC(=O)c2c(-c3cc(F)ccc3F)ccnc2C2CCC(F)(F)CC2)cn1] | (R)-2-(4,4-difluorocyclohexyl)-4- (2,5-difluorophenyl)-N-(2-(2- methylazetidin-1-yl)pyrimidin-5- yl)nicotinamide |
| 67 | 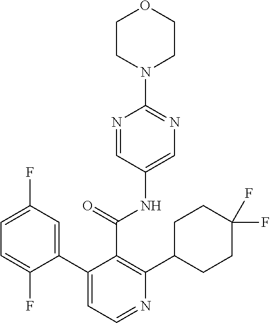 [SMILES: O=C(Nc1cnc(N2CCOCC2)nc1)c1c(-c2cc(F)ccc2F)ccnc1C1CCC(F)(F)CC1] | 2-(4,4-difluorocyclohexyl)-4-(2,5- difluorophenyl)-N-(2- morpholinopyrimidin-5- yl)nicotinamide |
| 68 | 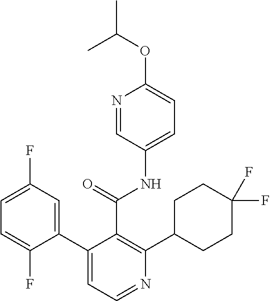 [SMILES: CC(C)Oc1ccc(NC(=O)c2c(-c3cc(F)ccc3F)ccnc2C2CCC(F)(F)CC2)cn1] | 2-(4,4-difluorocyclohexyl)-4-(2,5- difluorophenyl)-N-(6- isopropoxypyridin-3- yl)nicotinamide |
| 69 | 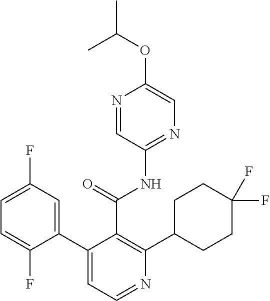 [SMILES: CC(C)Oc1cnc(NC(=O)c2c(-c3cc(F)ccc3F)ccnc2C2CCC(F)(F)CC2)cn1] | 2-(4,4-difluorocyclohexyl)-4-(2,5- difluorophenyl)-N-(5- isopropoxypyrazin-2- yl)nicotinamide |
| 70 | 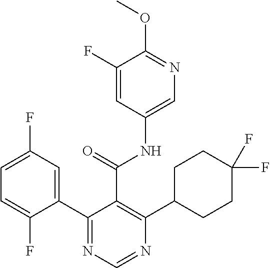 [SMILES: COc1ncc(NC(=O)c2c(-c3cc(F)ccc3F)ncnc2C2CCC(F)(F)CC2)cc1F] | 4-(4,4-difluorocyclohexyl)-6-(2,5- difluorophenyl)-N-(5-fluoro-6- methoxypyridin-3-yl)pyrimidine- 5-carboxamide |
| 71 | 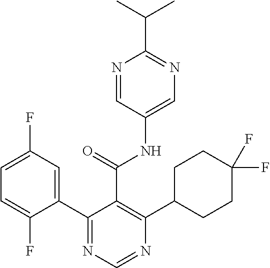 [SMILES: CC(C)c1ncc(NC(=O)c2c(-c3cc(F)ccc3F)ncnc2C2CCC(F)(F)CC2)cn1] | 4-(4,4-difluorocyclohexyl)-6-(2,5- difluorophenyl)-N-(2- isopropylpyrimidin-5- yl)pyrimidine-5-carboxamide |
| 72 | 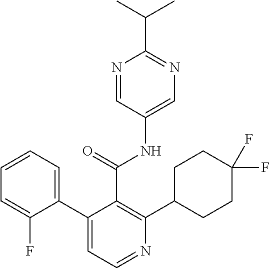 [SMILES: CC(C)c1ncc(NC(=O)c2c(-c3ccccc3F)ccnc2C2CCC(F)(F)CC2)cn1] | 2-(4,4-difluorocyclohexyl)-4-(2- fluorophenyl)-N-(2- isopropylpyrimidin-5- yl)nicotinamide |
| 73 | 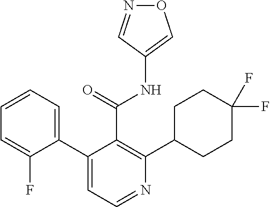 [SMILES: O=C(Nc1cnoc1)c1c(-c2ccccc2F)ccnc1C1CCC(F)(F)CC1] | 2-(4,4-difluorocyclohexyl)-4-(2- fluorophenyl)-N-(isoxazol-4- yl)nicotinamide |
| 74 | 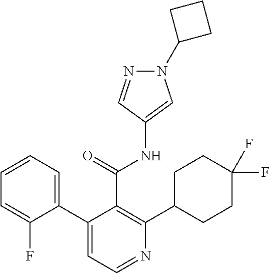 [SMILES: O=C(Nc1cnn(C2CCC2)c1)c1c(-c2ccccc2F)ccnc1C1CCC(F)(F)CC1] | N-(1-cyclobutyl-1H-pyrazol-4-yl)- 2-(4,4-difluorocyclohexyl)-4-(2- fluorophenyl)nicotinamide |
| 75 | 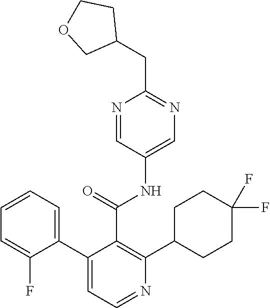 [SMILES: O=C(Nc1cnc(CC2CCOC2)nc1)c1c(-c2ccccc2F)ccnc1C1CCC(F)(F)CC1] | 2-(4,4-difluorocyclohexyl)-4-(2- fluorophenyl)-N-(2- ((tetrahydrofuran-3- yl)oxy)pyrimidin-5- yl)nicotinamide |
| 76 | 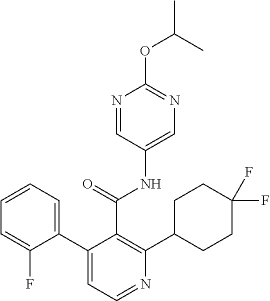 [SMILES: CC(C)Oc1ncc(NC(=O)c2c(-c3ccccc3F)ccnc2C2CCC(F)(F)CC2)cn1] | 2-(4,4-difluorocyclohexyl)-4-(2- fluorophenyl)-N-(2- isopropoxypyrimidin-5- yl)nicotinamide |
| 77 | 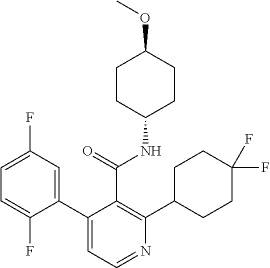 [SMILES: CO[C@H]1CC[C@H](NC(=O)c2c(-c3cc(F)ccc3F)ccnc2C2CCC(F)(F)CC2)CC1] | 2-(4,4-difluorocyclohexyl)-4- (2,5-difluorophenyl)-N-((1r,4r)-4- methoxycyclohexyl)nicotinamide |
| 78 | 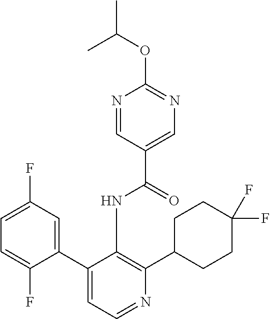 [SMILES: CC(C)Oc1ncc(C(=O)Nc2c(-c3cc(F)ccc3F)ccnc2C2CCC(F)(F)CC2)cn1] | N-(2-(4,4-difluorocyclohexyl)-4- (2,5-difluorophenyl)pyridin-3-yl)- 2-(2-methylprop-1-en-1- yl)pyrimidine-5-carboxamide |
| 79 | 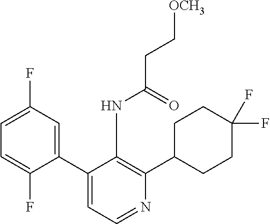 [SMILES: COCCC(=O)Nc1c(-c2cc(F)ccc2F)ccnc1C1CCC(F)(F)CC1] | N-(2-(4,4-difluorocyclohexyl)-4- (2,5-difluorophenyl)pyridin-3-yl)- 3-methoxypropanamide |
| 80 | 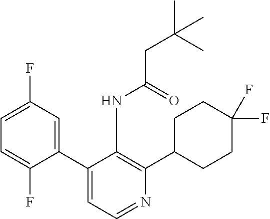 [SMILES: CC(C)(C)CC(=O)Nc1c(-c2cc(F)ccc2F)ccnc1C1CCC(F)(F)CC1] | N-(2-(4,4-difluorocyclohexyl)-4- (2,5-difluorophenyl)pyridin-3-yl)- 3,3-dimethylbutanamide |
| 81 | 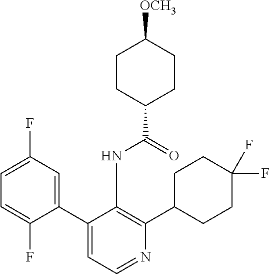 [SMILES: CO[C@H]1CC[C@H](C(=O)Nc2c(-c3cc(F)ccc3F)ccnc2C2CCC(F)(F)CC2)CC1] | (1r,4r)-N-(2-(4,4- difluorocyclohexyl)-4-(2,5- difluorophenyl)pyridin-3-yl)-4- methoxycyclohexane-1- carboxamide |
| 82 | 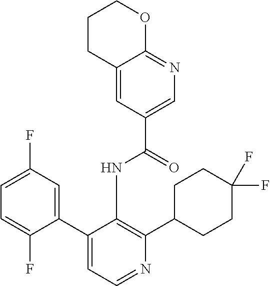 [SMILES: O=C(Nc1c(-c2cc(F)ccc2F)ccnc1C1CCC(F)(F)CC1)c1cnc2c(c1)CCCO2] | N-(2-(4,4-difluorocyclohexyl)-4- (2,5-difluorophenyl)pyridin-3-yl)- 3,4-dihydro-2H-pyrano[2,3- b]pyridine-6-carboxamide |
| 83 | 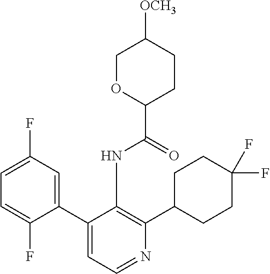 [SMILES: COC1CCC(C(=O)Nc2c(-c3cc(F)ccc3F)ccnc2C2CCC(F)(F)CC2)OC1] | N-(2-(4,4-difluorocyclohexyl)-4- (2,5-difluorophenyl)pyridin-3-yl)- 5-methoxytetrahydro-2H-pyran- 2-carboxamide |
| 84 | 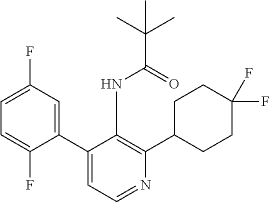 [SMILES: CC(C)(C)C(=O)Nc1c(-c2cc(F)ccc2F)ccnc1C1CCC(F)(F)CC1] | N-(2-(4,4-difluorocyclohexyl)-4- (2,5-difluorophenyl)pyridin-3- yl)pivalamide |
| 85 | 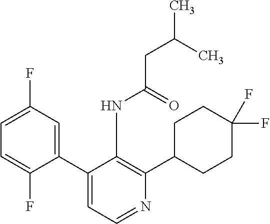 [SMILES: CC(C)CC(=O)Nc1c(-c2cc(F)ccc2F)ccnc1C1CCC(F)(F)CC1] | N-(2-(4,4-difluorocyclohexyl)-4- (2,5-difluorophenyl)pyridin-3-yl)- 3-methylbutanamide |
| 86 | 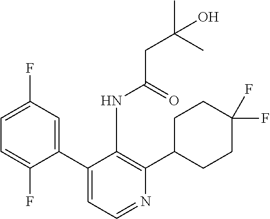 [SMILES: CC(C)(O)CC(=O)Nc1c(-c2cc(F)ccc2F)ccnc1C1CCC(F)(F)CC1] | N-(2-(4,4-difluorocyclohexyl)-4- (2,5-difluorophenyl)pyridin-3-yl)- 3-hydroxy-3-methylbutanamide |
| 87 | 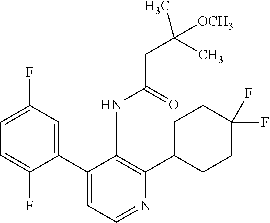 [SMILES: COC(C)(C)CC(=O)Nc1c(-c2cc(F)ccc2F)ccnc1C1CCC(F)(F)CC1] | N-(2-(4,4-difluorocyclohexyl)-4- (2,5-difluorophenyl)pyridin-3-yl)- 3-methoxy-3-methylbutanamide |
| 88 | 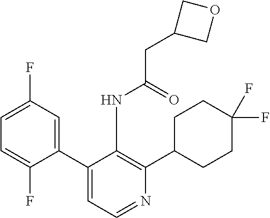 [SMILES: O=C(CC1COC1)Nc1c(-c2cc(F)ccc2F)ccnc1C1CCC(F)(F)CC1] | N-(2-(4,4-difluorocyclohexyl)-4- (2,5-difluorophenyl)pyridin-3-yl)- 2-(oxetan-3-yl)acetamide |
| 89 | 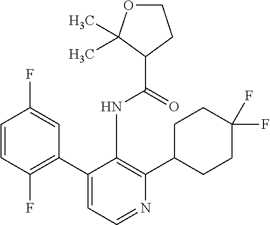 [SMILES: CC1(C)OCCC1C(=O)Nc1c(-c2cc(F)ccc2F)ccnc1C1CCC(F)(F)CC1] | N-(2-(4,4-difluorocyclohexyl)-4- (2,5-difluorophenyl)pyridin-3-yl)- 2,2-dimethyltetrahydrofuran-3- carboxamide |
| 90 | 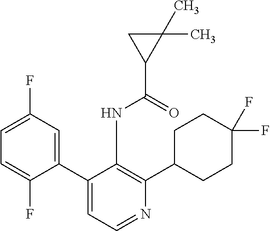 [SMILES: CC1(C)CC1C(=O)Nc1c(-c2cc(F)ccc2F)ccnc1C1CCC(F)(F)CC1] | N-(2-(4,4-difluorocyclohexyl)-4- (2,5-difluorophenyl)pyridin-3-yl)- 2,2-dimethylcyclopropane-1- carboxamide |
| 91 | 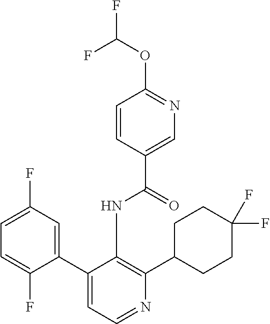 [SMILES: O=C(Nc1c(-c2cc(F)ccc2F)ccnc1C1CCC(F)(F)CC1)c1ccc(OC(F)F)nc1] | 5-chloro-4′-(4-methylpiperazin-1- yl)-[1,1′-biphenyl]-2-ol |
| 92 | 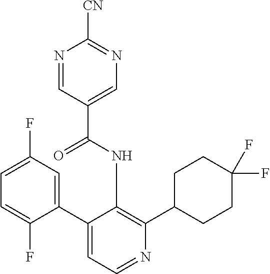 [SMILES: N#Cc1ncc(C(=O)Nc2c(-c3cc(F)ccc3F)ccnc2C2CCC(F)(F)CC2)cn1] | 2-cyano-N-(2-(4,4- difluorocyclohexyl)-4-(2,5- difluorophenyl)pyridin-3- yl)pyrimidine-5-carboxamide |
| 93 | 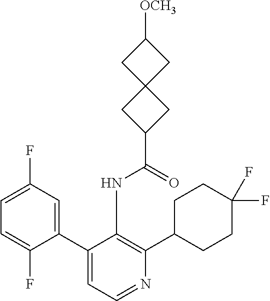 [SMILES: COC1CC2(C1)CC(C(=O)Nc1c(-c3cc(F)ccc3F)ccnc1C1CCC(F)(F)CC1)C2] | N-(2-(4,4-difluorocyclohexyl)-4- (2,5-difluorophenyl)pyridin-3-yl)- 6-methoxyspiro[3.3]heptane-2- carboxamide |
| 94 | 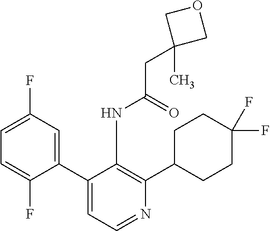 [SMILES: CC1(CC(=O)Nc2c(-c3cc(F)ccc3F)ccnc2C2CCC(F)(F)CC2)COC1] | N-(2-(4,4-difluorocyclohexyl)-4- (2,5-difluorophenyl)pyridin-3-yl)- 2-(3-methyloxetan-3- yl)acetamide |
| 95 | 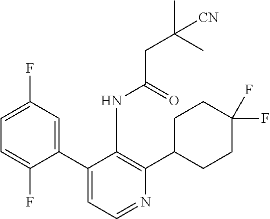 [SMILES: CC(C)(C#N)CC(=O)Nc1c(-c2cc(F)ccc2F)ccnc1C1CCC(F)(F)CC1] | N-(2-(4,4-difluorocyclohexyl)-4- (2,5-difluorophenyl)pyridin-3-yl)- 2-(oxetan-3-yl)acetamide |
| 96 | 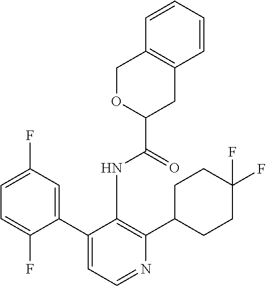 [SMILES: O=C(Nc1c(-c2cc(F)ccc2F)ccnc1C1CCC(F)(F)CC1)C1Cc2ccccc2CO1] | N-(2-(4,4-difluorocyclohexyl)-4- (2,5-difluorophenyl)pyridin-3- yl)isochromane-3-carboxamide |
| 97 | 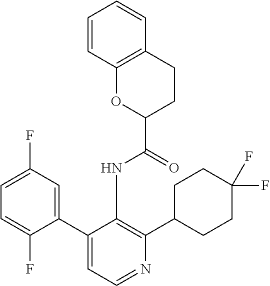 [SMILES: O=C(Nc1c(-c2cc(F)ccc2F)ccnc1C1CCC(F)(F)CC1)C1CCc2ccccc2O1] | N-(2-(4,4-difluorocyclohexyl)-4- (2,5-difluorophenyl)pyridin-3- yl)isochromane-3-carboxamide |
| 98 | 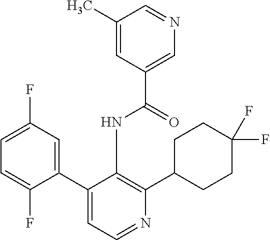 [SMILES: Cc1cncc(C(=O)Nc2c(-c3cc(F)ccc3F)ccnc2C2CCC(F)(F)CC2)c1] | N-(2-(4,4-difluorocyclohexyl)-4- (2,5-difluorophenyl)pyridin-3-yl)- 5-methylnicotinamide |
| 99 | 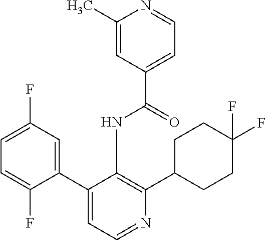 [SMILES: Cc1cc(C(=O)Nc2c(-c3cc(F)ccc3F)ccnc2C2CCC(F)(F)CC2)ccn1] | N-(2-(4,4-difluorocyclohexyl)-4- (2,5-difluorophenyl)pyridin-3-yl)- 2-methylisonicotinamide |
| 100 | 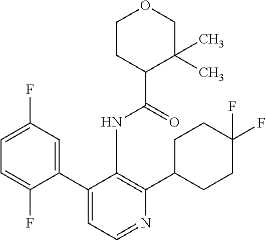 [SMILES: CC1(C)COCCC1C(=O)Nc1c(-c2cc(F)ccc2F)ccnc1C1CCC(F)(F)CC1] | N-(2-(4,4-difluorocyclohexyl)-4- (2,5-difluorophenyl)pyridin-3-yl)- 3,3-dimethyltetrahydro-2H- pyran-4-carboxamide |
| 101 | 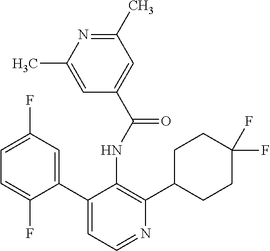 [SMILES: Cc1cc(C(=O)Nc2c(-c3cc(F)ccc3F)ccnc2C2CCC(F)(F)CC2)cc(C)n1] | N-(2-(4,4-difluorocyclohexyl)-4- (2,5-difluorophenyl)-pyridin-3- yl)-2,6-dimethylisonicotinamide |
| 102 | 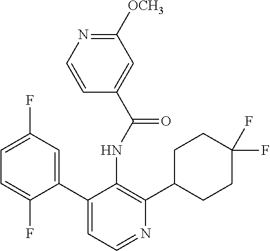 [SMILES: COc1cc(C(=O)Nc2c(-c3cc(F)ccc3F)ccnc2C2CCC(F)(F)CC2)ccn1] | N-(2-(4,4-difluorocyclohexyl)-4- (2,5-difluorophenyl)-pyridin-3- yl)-2-methoxyisonicotinamide |
| 103 | 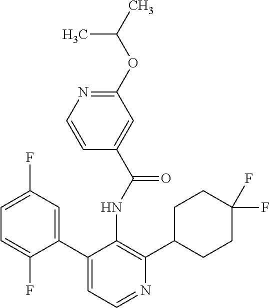 [SMILES: CC(C)Oc1cc(C(=O)Nc2c(-c3cc(F)ccc3F)ccnc2C2CCC(F)(F)CC2)ccn1] | N-(2-(4,4-difluorocyclohexyl)-4- (2,5-difluorophenyl)pyridin-3-yl)- 5-isopropoxynicotinamide |
| 104 | 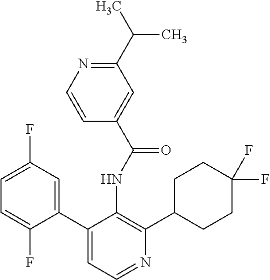 [SMILES: CC(C)c1cc(C(=O)Nc2c(-c3cc(F)ccc3F)ccnc2C2CCC(F)(F)CC2)ccn1] | N-(2-(4,4-difluorocyclohexyl)-4- (2,5-difluorophenyl)pyridin-3-yl)- 2-isopropylisonicotinamide |
| 105 | 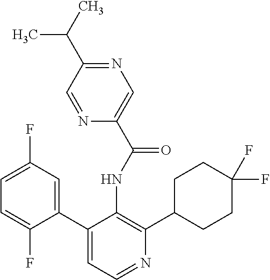 [SMILES: CC(C)c1cnc(C(=O)Nc2c(-c3cc(F)ccc3F)ccnc2C2CCC(F)(F)CC2)cn1] | N-(2-(4,4-difluorocyclohexyl)-4- (2,5-difluorophenyl)pyridin-3-yl)- 5-isopropylpyrazine-2- carboxamide |
| 106 | 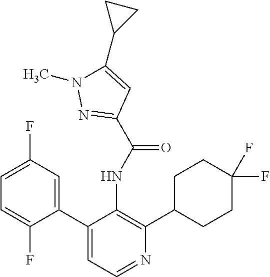 [SMILES: Cn1nc(C(=O)Nc2c(-c3cc(F)ccc3F)ccnc2C2CCC(F)(F)CC2)cc1C1CC1] | 5-cyclopropyl-N-(2-(4,4- difluorocyclohexyl)-4-(2,5- difluorophenyl)pyridin-3-yl)-1- methyl-1H-pyrazole-3- carboxamide |
| 107 | 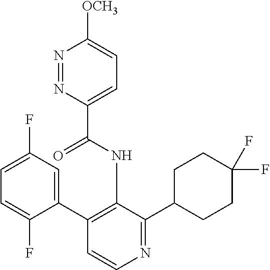 [SMILES: COc1ccc(C(=O)Nc2c(-c3cc(F)ccc3F)ccnc2C2CCC(F)(F)CC2)nn1] | N-(2-(4,4-difluorocyclohexyl)-4- (2,5-difluorophenyl)pyridin-3-yl)- 6-methoxypyridazine-3- carboxamide |
| 108 | 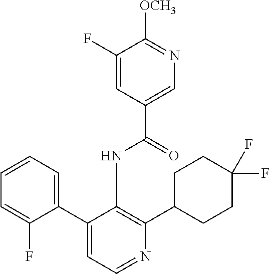 [SMILES: COc1ncc(C(=O)Nc2c(-c3ccccc3F)ccnc2C2CCC(F)(F)CC2)cc1F] | N-(2-(4,4-difluorocyclohexyl)-4- (2-fluorophenyl)pyridin-3-yl)-5- fluoro-6-methoxynicotinamide |
| 109 | 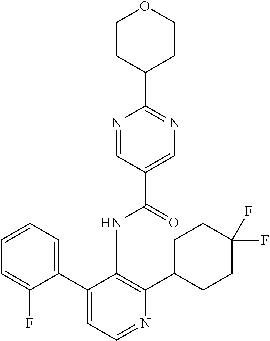 [SMILES: O=C(Nc1c(-c2ccccc2F)ccnc1C1CCC(F)(F)CC1)c1cnc(C2CCOCC2)nc1] | N-(2-(4,4-difluorocyclohexyl)-4- (2-fluorophenyl)pyridin-3-yl)-2- (tetrahydro-2H-pyran-4-yl) pyrimidine-5-carboxamide |
| 110 | 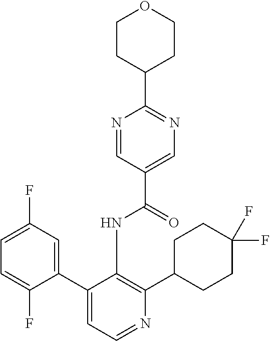 [SMILES: O=C(Nc1c(-c2cc(F)ccc2F)ccnc1C1CCC(F)(F)CC1)c1cnc(C2CCOCC2)nc1] | N-(2-(4,4-difluorocyclohexyl)-4- (2,5-difluorophenyl)pyridin-3-yl)- 2-(tetrahydro-2H-pyran-4- yl)pyrimidine-5-carboxamide |
| 111 | 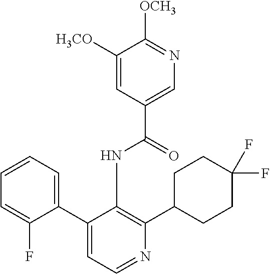 [SMILES: COc1cc(C(=O)Nc2c(-c3ccccc3F)ccnc2C2CCC(F)(F)CC2)cnc1OC] | N-(2-(4,4-difluorocyclohexyl)-4- (2-fluorophenyl)pyridin-3-yl)-5,6- dimethoxynicotinamide |
| 112 | 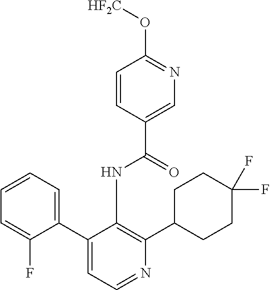 | N-(2-(4,4-difluorocyclohexyl)-4- (2-fluorophenyl)pyridin-3-yl)-6- (difluoromethoxy)nicotinamide |
| 113 | 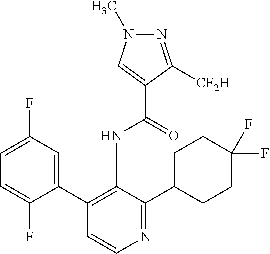 [SMILES: Cn1cc(C(=O)Nc2c(-c3cc(F)ccc3F)ccnc2C2CCC(F)(F)CC2)c(C(F)F)n1] | N-(2-(4,4-difluorocyclohexyl)-4- (2,5-difluorophenyl)pyridin-3-yl)- 3-(difluoromethyl)-1-methyl-1H- pyrazole-4-carboxamide |
| 114 | 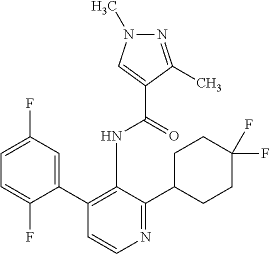 [SMILES: Cc1nn(C)cc1C(=O)Nc1c(-c2cc(F)ccc2F)ccnc1C1CCC(F)(F)CC1] | N-(2-(4,4-difluorocyclohexyl)-4- (2,5-difluorophenyl)pyridin-3-yl)- 1,3-dimethyl-1H-pyrazole-4- carboxamide |
| 115 | 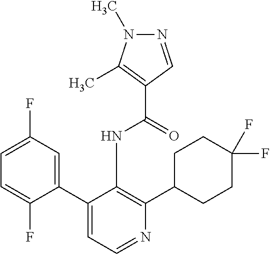 [SMILES: Cc1c(C(=O)Nc2c(-c3cc(F)ccc3F)ccnc2C2CCC(F)(F)CC2)cnn1C] | N-(2-(4,4-difluorocyclohexyl)-4- (2,5-difluorophenyl)pyridin-3-yl)- 1,5-dimethyl-1H-pyrazole-4- carboxamide |
| 116 | 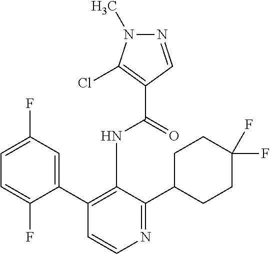 [SMILES: Cn1ncc(C(=O)Nc2c(-c3cc(F)ccc3F)ccnc2C2CCC(F)(F)CC2)c1Cl] | 5-chloro-N-(2-(4,4- difluorocyclohexyl)-4-(2,5- difluorophenyl)pyridin-3-yl)-1- methyl-1H-pyrazole-4- carboxamide |
| 117 | 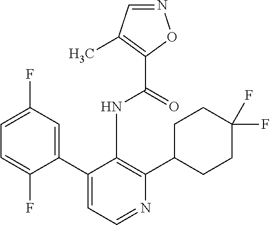 [SMILES: Cc1cnoc1C(=O)Nc1c(-c2cc(F)ccc2F)ccnc1C1CCC(F)(F)CC1] | N-(2-(4,4-difluorocyclohexyl)-4- (2,5-difluorophenyl)pyridin-3-yl)- 4-methylisoxazole-5- carboxamide |
| 118 | 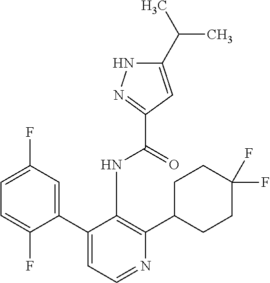 [SMILES: CC(C)c1cc(C(=O)Nc2c(-c3cc(F)ccc3F)ccnc2C2CCC(F)(F)CC2)n[nH]1] | N-(2-(4,4-difluorocyclohexyl)-4- (2,5-difluorophenyl)pyridin-3-yl)- 5-isopropyl-1H-pyrazole-3- carboxamide |
| 119 | 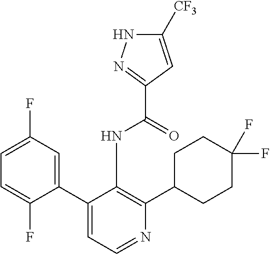 [SMILES: O=C(Nc1c(-c2cc(F)ccc2F)ccnc1C1CCC(F)(F)CC1)c1cc(C(F)(F)F)[nH]n1] | N-(2-(4,4-difluorocyclohexyl)-4- (2,5-difluorophenyl)pyridin-3-yl)- 5-(trifluoromethyl)-1H-pyrazole- 3-carboxamide |
| 120 | 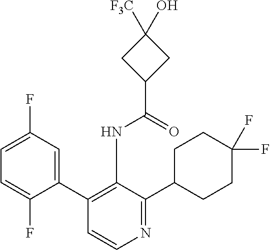 [SMILES: O=C(Nc1c(-c2cc(F)ccc2F)ccnc1C1CCC(F)(F)CC1)C1CC(O)(C(F)(F)F)C1] | N-(2-(4,4-difluorocyclohexyl)-4- (2,5-difluorophenyl)pyridin-3-yl)- 3-hydroxy-3- (trifluoromethyl)cyclobutane-1- carboxamide |
| 121 | 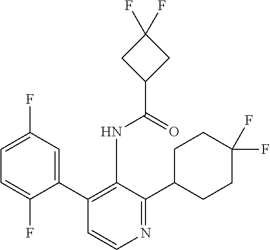 [SMILES: O=C(Nc1c(-c2cc(F)ccc2F)ccnc1C1CCC(F)(F)CC1)C1CC(F)(F)C1] | N-(2-(4,4-difluorocyclohexyl)-4- (2,5-difluorophenyl)pyridin-3-yl)- 3,3-difluorocyclobutane-1- carboxamide |
| 122 | 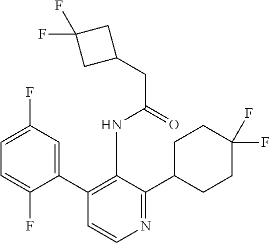 [SMILES: O=C(CC1CC(F)(F)C1)Nc1c(-c2cc(F)ccc2F)ccnc1C1CCC(F)(F)CC1] | 2-(3,3-difluorocyclobutyl)-N-(2- (4,4-difluorocyclohexyl)-4-(2,5- difluorophenyl)pyridin-3- yl)acetamide |
| 123 | 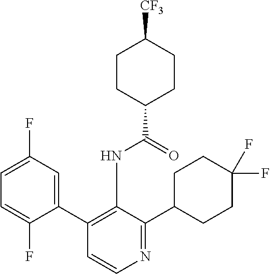 [SMILES: O=C(Nc1c(-c2cc(F)ccc2F)ccnc1C1CCC(F)(F)CC1)[C@H]1CC[C@H](C(F)(F)F)CC1] | (1r,4r)-N-(2-(4,4- difluorocyclohexyl)-4-(2,5- difluorophenyl)pyridin-3-yl)-4- (trifluoromethyl)cyclohexane-1- carboxamide |
| 124 | 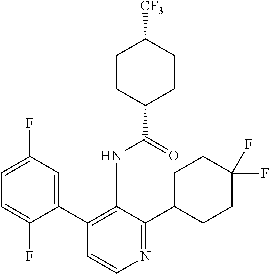 [SMILES: O=C(Nc1c(-c2cc(F)ccc2F)ccnc1C1CCC(F)(F)CC1)[C@H]1CC[C@@H](C(F)(F)F)CC1] | (1r,3r)-N-(2-(4,4- difluorocyclohexyl)-4-(2,5- difluorophenyl)pyridin-3-yl)-3- (trifluoromethyl)cyclobutane-1- carboxamide |
| 125 | 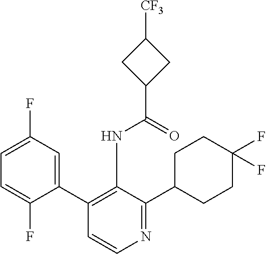 [SMILES: O=C(Nc1c(-c2cc(F)ccc2F)ccnc1C1CCC(F)(F)CC1)C1CC(C(F)(F)F)C1] | N-(2-(4,4-difluorocyclohexyl)-4- (2,5-difluorophenyl)pyridin-3-yl)- 3-(trifluoromethyl)cyclobutane-1- carboxamide (racemic mix) |
| 126 | 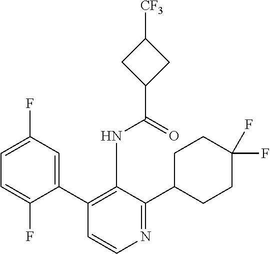 [SMILES: O=C(Nc1c(-c2cc(F)ccc2F)ccnc1C1CCC(F)(F)CC1)C1CC(C(F)(F)F)C1] | N-(2-(4,4-difluorocyclohexyl)-4- (2,5-difluorophenyl)pyridin-3-yl)- 3-(trifluoromethyl)cyclobutane-1- carboxamide P1 |
| 127 | 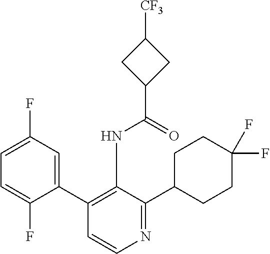 [SMILES: O=C(Nc1c(-c2cc(F)ccc2F)ccnc1C1CCC(F)(F)CC1)C1CC(C(F)(F)F)C1] | N-(2-(4,4-difluorocyclohexyl)-4- (2,5-difluorophenyl)pyridin-3-yl)- 3-(trifluoromethyl)cyclobutane-1- carboxamide P2 |
| 128 | 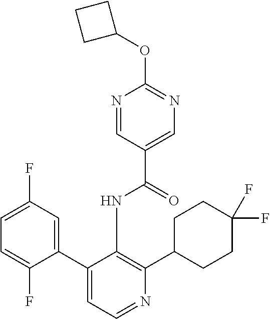 [SMILES: O=C(Nc1c(-c2cc(F)ccc2F)ccnc1C1CCC(F)(F)CC1)c1cnc(OC2CCC2)nc1] | N-(2-(4,4-difluorocyclohexyl)-4- (2,5-difluorophenyl)pyridin-3-yl)- 2-(2-methylprop-1-en-1- yl)pyrimidine-5-carboxamide |
| 129 | 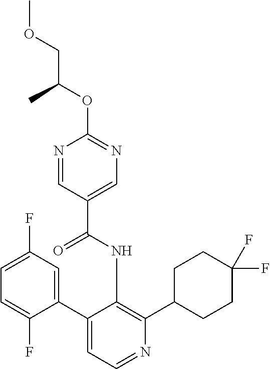 [SMILES: COC[C@H](C)Oc1ncc(C(=O)Nc2c(-c3cc(F)ccc3F)ccnc2C2CCC(F)(F)CC2)cn1] | (S)-N-(2-(4,4-difluorocyclohexyl)- 4-(2,5-difluorophenyl)pyridin-3- yl)-2-((1-methoxypropan-2- yl)oxy)pyrimidine-5-carboxamide |
| 130 | 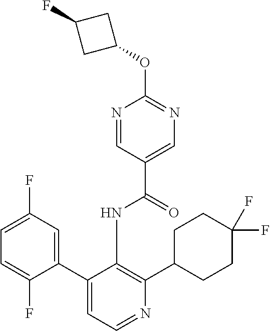 [SMILES: O=C(Nc1c(-c2cc(F)ccc2F)ccnc1C1CCC(F)(F)CC1)c1cnc(O[C@H]2C[C@H](F)C2)nc1] | trans-N-(2-(4,4- difluorocyclohexyl)-4-(2,5- difluorophenyl)pyridin-3-yl)-2- ((1r,3r)-3- fluorocyclobutoxy)pyrimidine-5- carboxamide |
| 131 | 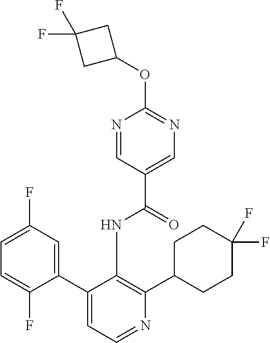 [SMILES: O=C(Nc1c(-c2cc(F)ccc2F)ccnc1C1CCC(F)(F)CC1)c1cnc(OC2CC(F)(F)C2)nc1] | 2-(3,3-difluorocyclobutoxy)-N-(2- (4,4-difluorocyclohexyl)-4-(2,5- difluorophenyl)pyridin-3- yl)pyrimidine-5-carboxamide |
| 132 | 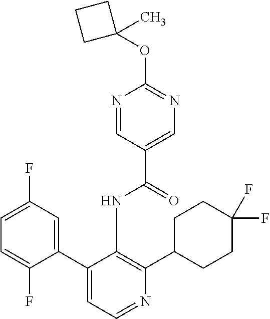 [SMILES: CC1(Oc2ncc(C(=O)Nc3c(-c4cc(F)ccc4F)ccnc3C3CCC(F)(F)CC3)cn2)CCC1] | N-(2-(4,4-difluorocyclohexyl)-4- (2,5-difluorophenyl)pyridin-3-yl)- 2-(1-methylcyclobutoxy) pyrimidine-5-carboxamide |
| 133 | 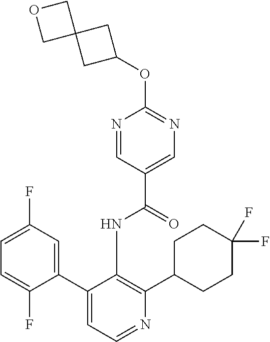 [SMILES: O=C(Nc1c(-c2cc(F)ccc2F)ccnc1C1CCC(F)(F)CC1)c1cnc(OC2CC3(COC3)C2)nc1] | 2-((2-oxaspiro[3.3]heptan-6- yl)oxy)-N-(2-(4,4- difluorocyclohexyl)-4-(2,5- difluorophenyl)pyridin-3- yl)pyrimidine-5-carboxamide |
| 134 | 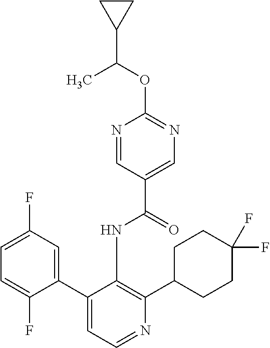 [SMILES: CC(Oc1ncc(C(=O)Nc2c(-c3cc(F)ccc3F)ccnc2C2CCC(F)(F)CC2)cn1)C1CC1] | 2-(1-cyclopropylethoxy)-N-(2- (4,4-difluorocyclohexyl)-4-(2,5- difluorophenyl)pyridin-3- yl)pyrimidine-5-carboxamide |
| 135 | 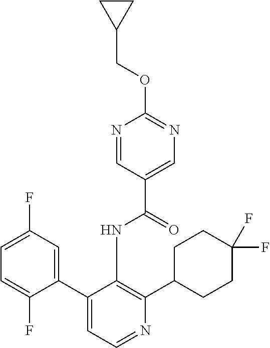 [SMILES: O=C(Nc1c(-c2cc(F)ccc2F)ccnc1C1CCC(F)(F)CC1)c1cnc(OCC2CC2)nc1] | 2-(cyclopropylmethoxy)-N-(2- (4,4-difluorocyclohexyl)-4-(2,5- difluorophenyl)pyridin-3- yl)pyrimidine-5-carboxamide |
| 136 | 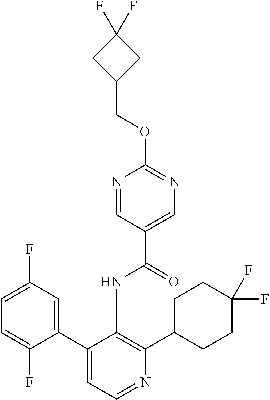 [SMILES: O=C(Nc1c(-c2cc(F)ccc2F)ccnc1C1CCC(F)(F)CC1)c1cnc(OCC2CC(F)(F)C2)nc1] | 2-((3,3- difluorocyclobutyl)methoxy)-N- (2-(4,4-difluorocyclohexyl)-4- (2,5-difluorophenyl)pyridin-3- yl)pyrimidine-5-carboxamide |
| 137 | 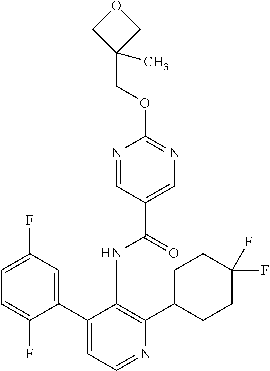 [SMILES: CC1(COc2ncc(C(=O)Nc3c(-c4cc(F)ccc4F)ccnc3C3CCC(F)(F)CC3)cn2)COC1] | N-(2-(4,4-difluorocyclohexyl)-4- (2,5-difluorophenyl)pyridin-3-yl)- 2-((3-methyloxetan-3- yl)methoxy)pyrimidine-5- carboxamide |
| 138 | 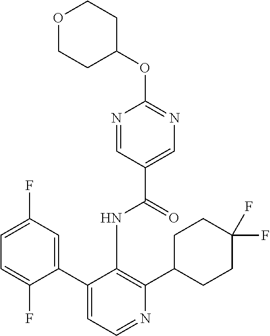 [SMILES: O=C(Nc1c(-c2cc(F)ccc2F)ccnc1C1CCC(F)(F)CC1)c1cnc(OC2CCOCC2)nc1] | N-(2-(4,4-difluorocyclohexyl)-4- (2,5-difluorophenyl)pyridin-3-yl)- 2-((tetrahydro-2H-pyran-4- yl)oxy)pyrimidine-5-carboxamide |
| 139 | 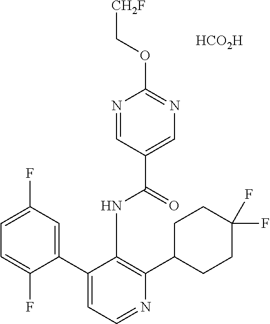 | N-(2-(4,4-difluorocyclohexyl)-4- (2,5-difluorophenyl)pyridin-3-yl)- 2-(2-fluoroethoxy)pyrimidine-5- carboxamide formate salt |
| 140 | 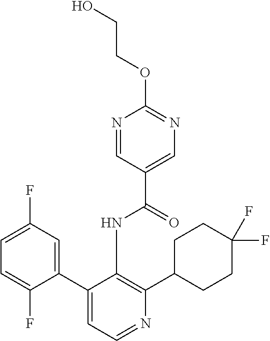 [SMILES: O=C(Nc1c(-c2cc(F)ccc2F)ccnc1C1CCC(F)(F)CC1)c1cnc(OCCO)nc1] | N-(2-(4,4-difluorocyclohexyl)-4- (2,5-difluorophenyl)pyridin-3-yl)- 2-(2-hydroxyethoxy)pyrimidine-5- carboxamide |
| 141 | 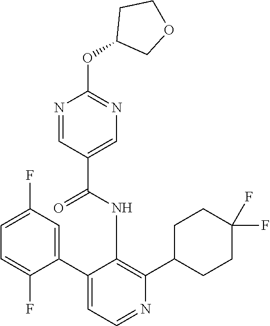 [SMILES: O=C(Nc1c(-c2cc(F)ccc2F)ccnc1C1CCC(F)(F)CC1)c1cnc(O[C@@H]2CCOC2)nc1] | (R)-N-(2-(4,4-difluorocyclohexyl)- 4-(2,5-difluorophenyl)pyridin-3- yl)-2-((tetrahydrofuran-3- yl)oxy)pyrimidine-5-carboxamide |
| 142 | 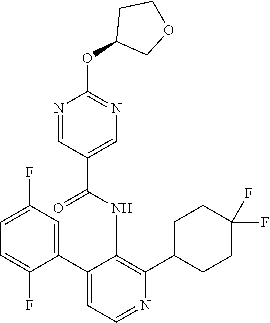 [SMILES: O=C(Nc1c(-c2cc(F)ccc2F)ccnc1C1CCC(F)(F)CC1)c1cnc(O[C@H]2CCOC2)nc1] | (S)-N-(2-(4,4-difluorocyclohexyl)- 4-(2,5-difluorophenyl)pyridin-3- yl)-2-((tetrahydrofuran-3- yl)oxy)pyrimidine-5-carboxamide |
| 143 | 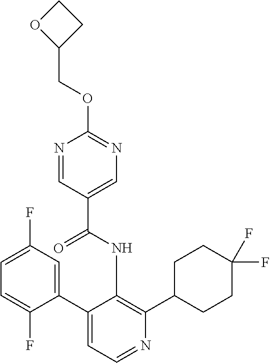 [SMILES: O=C(Nc1c(-c2cc(F)ccc2F)ccnc1C1CCC(F)(F)CC1)c1cnc(OCC2CCO2)nc1] | N-(2-(4,4-difluorocyclohexyl)-4- (2,5-difluorophenyl)pyridin-3-yl)- 2-(oxetan-2- ylmethoxy)pyrimidine-5- carboxamide |
| 144 | 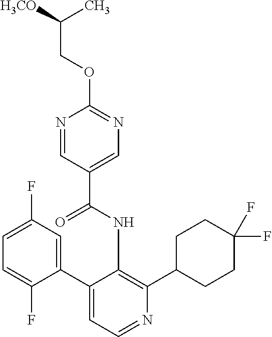 [SMILES: CO[C@@H](C)COc1ncc(C(=O)Nc2c(-c3cc(F)ccc3F)ccnc2C2CCC(F)(F)CC2)cn1] | (S)-N-(2-(4,4-difluorocyclohexyl)- 4-(2,5-difluorophenyl)pyridin-3- yl)-2-(2- methoxypropoxy)pyrimidine-5- carboxamide |
| 145 | 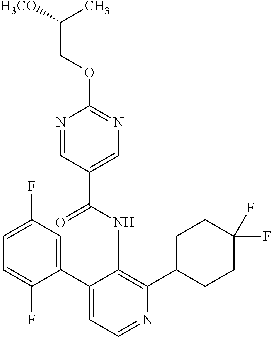 [SMILES: CO[C@H](C)COc1ncc(C(=O)Nc2c(-c3cc(F)ccc3F)ccnc2C2CCC(F)(F)CC2)cn1] | (R)-N-(2-(4,4-difluorocyclohexyl)- 4-(2,5-difluorophenyl)pyridin-3- yl)-2-(2- methoxypropoxy)pyrimidine-5- carboxamide |
| 146 | 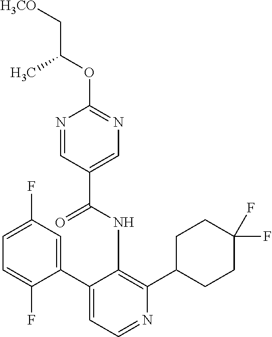 [SMILES: COC[C@@H](C)Oc1ncc(C(=O)Nc2c(-c3cc(F)ccc3F)ccnc2C2CCC(F)(F)CC2)cn1] | (R)-N-(2-(4,4-difluorocyclohexyl)- 4-(2,5-difluorophenyl)pyridin-3- yl)-2-((1-methoxypropan-2- yl)oxy)pyrimidine-5-carboxamide |
| 147 | 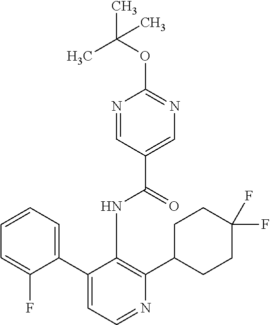 [SMILES: CC(C)(C)Oc1ncc(C(=O)Nc2c(-c3ccccc3F)ccnc2C2CCC(F)(F)CC2)cn1] | 2-(tert-butoxy)-N-(2-(4,4- difluorocyclohexyl)-4-(2- fluorophenyl)pyridin-3- yl)pyrimidine-5-carboxamide |
| 148 | 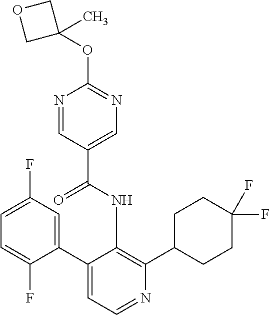 [SMILES: CC1(Oc2ncc(C(=O)Nc3c(-c4cc(F)ccc4F)ccnc3C3CCC(F)(F)CC3)cn2)COC1] | N-(2-(4,4-difluorocyclohexyl)-4- (2,5-difluorophenyl)pyridin-3-yl)- 2-((3-methyloxetan-3- yl)oxy)pyrimidine-5-carboxamide |
| 149 | 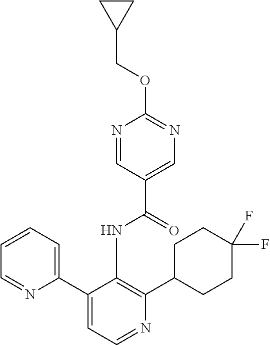 [SMILES: O=C(Nc1c(-c2ccccn2)ccnc1C1CCC(F)(F)CC1)c1cnc(OCC2CC2)nc1] | 2-(cyclopropylmethoxy)-N-(2′- (4,4-difluorocyclohexyl)-[2,4′- bipyridin]-3′-yl)pyrimidine-5- carboxamide |
| 150 | 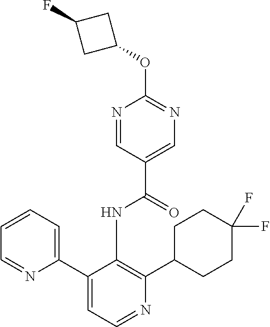 [SMILES: O=C(Nc1c(-c2ccccn2)ccnc1C1CCC(F)(F)CC1)c1cnc(O[C@H]2C[C@H](F)C2)nc1] | N-(2′-(4,4-difluorocyclohexyl)- [2,4′-bipyridin]-3′-yl)-2-((1r,3r)-3- fluorocyclobutoxy)pyrimidine-5- carboxamide |
| 151 | 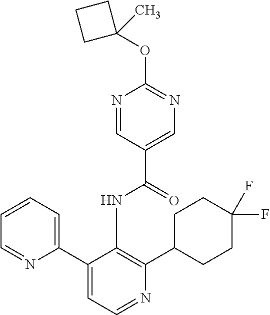 [SMILES: CC1(Oc2ncc(C(=O)Nc3c(-c4ccccn4)ccnc3C3CCC(F)(F)CC3)cn2)CCC1] | N-(2′-(4,4-difluorocyclohexyl)- [2,4′-bipyridin]-3′-yl)-2-(1- methylcyclobutoxy)pyrimidine-5- carboxamide |
| 152 | 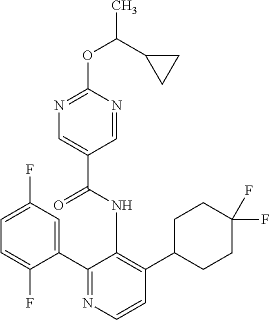 [SMILES: CC(Oc1ncc(C(=O)Nc2c(C3CCC(F)(F)CC3)ccnc2-c2cc(F)ccc2F)cn1)C1CC1] | 2-(1-cyclopropylethoxy)-N-(4- (4,4-difluorocyclohexyl)-2-(2,5- difluorophenyl)pyridin-3- yl)pyrimidine-5-carboxamide |
| 153 | 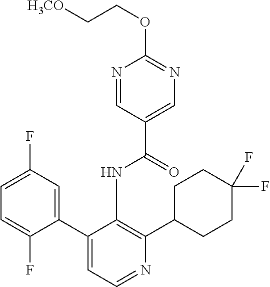 [SMILES: COCCOc1ncc(C(=O)Nc2c(-c3cc(F)ccc3F)ccnc2C2CCC(F)(F)CC2)cn1] | N-(2-(4,4-difluorocyclohexyl)-4- (2,5-difluorophenyl)pyridin-3-yl)- 2-(2-methoxyethoxy)pyrimidine- 5-carboxamide |
| 154 | 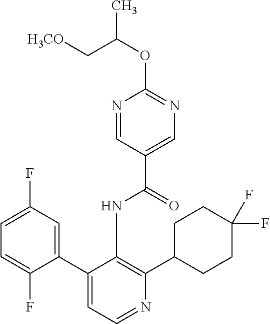 [SMILES: COCC(C)Oc1ncc(C(=O)Nc2c(-c3cc(F)ccc3F)ccnc2C2CCC(F)(F)CC2)cn1] | N-(2-(4,4-difluorocyclohexyl)-4- (2,5-difluorophenyl)pyridin-3-yl)- 2-((1-methoxypropan-2- yl)oxy)pyrimidine-5-carboxamide |
| 155 | 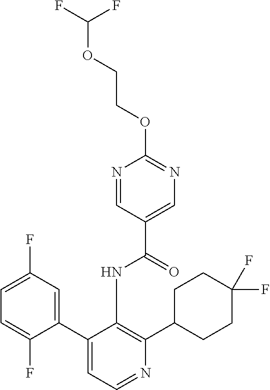 [SMILES: O=C(Nc1c(-c2cc(F)ccc2F)ccnc1C1CCC(F)(F)CC1)c1cnc(OCCOC(F)F)nc1] | N-(2-(4,4-difluorocyclohexyl)-4- (2,5-difluorophenyl)pyridin-3-yl)- 2-(2-(difluoromethoxy)ethoxy) pyrimidine-5-carboxamide |
| 156 | 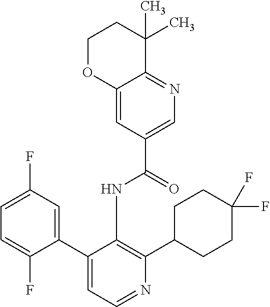 [SMILES: CC1(C)CCOc2cc(C(=O)Nc3c(-c4cc(F)ccc4F)ccnc3C3CCC(F)(F)CC3)cnc21] | N-(2-(4,4-difluorocyclohexyl)-4- (2,5-difluorophenyl)pyridin-3-yl)- 2-(2-(difluoromethoxy)ethoxy) pyrimidine-5-carboxamide |
| 157 | 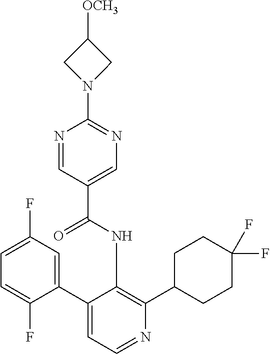 [SMILES: COC1CN(c2ncc(C(=O)Nc3c(-c4cc(F)ccc4F)ccnc3C3CCC(F)(F)CC3)cn2)C1] | N-(2-(4,4-difluorocyclohexyl)-4- (2,5-difluorophenyl)pyridin-3-yl)- 2-(3-methoxyazetidin-1- yl)pyrimidine-5-carboxamide |
| 158 | 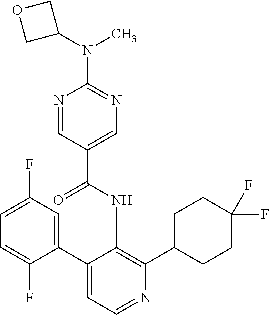 [SMILES: CN(c1ncc(C(=O)Nc2c(-c3cc(F)ccc3F)ccnc2C2CCC(F)(F)CC2)cn1)C1COC1] | N-(2-(4,4-difluorocyclohexyl)-4- (2,5-difluorophenyl)pyridin-3-yl)- 2-(methyl(oxetan-3- yl)amino)pyrimidine-5- carboxamide |
| 159 | 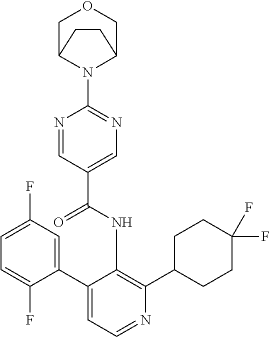 [SMILES: O=C(Nc1c(-c2cc(F)ccc2F)ccnc1C1CCC(F)(F)CC1)c1cnc(N2C3CCC2COC3)nc1] | 2-(3-oxa-8- azabicyclo[3.2.1]octan-8-yl)-N- (2-(4,4-difluorocyclohexyl)-4- (2,5-difluorophenyl)pyridin-3- yl)pyrimidine-5-carboxamide |
| 160 | 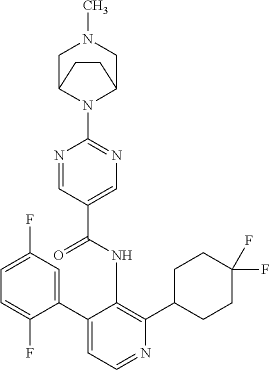 [SMILES: CN1CC2CCC(C1)N2c1ncc(C(=O)Nc2c(-c3cc(F)ccc3F)ccnc2C2CCC(F)(F)CC2)cn1] | N-(2-(4,4-difluorocyclohexyl)-4- (2,5-difluorophenyl)pyridin-3-yl)- 2-((1R,5S)-3-methyl-3,8- diazabicyclo[3.2.1]octan-8- yl)pyrimidine-5-carboxamide |
| 161 | 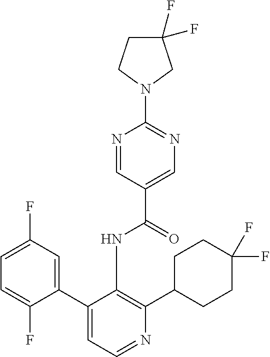 [SMILES: O=C(Nc1c(-c2cc(F)ccc2F)ccnc1C1CCC(F)(F)CC1)c1cnc(N2CCC(F)(F)C2)nc1] | N-(2-(4,4-difluorocyclohexyl)-4- (2,5-difluorophenyl)pyridin-3-yl)- 2-(3,3-difluoropyrrolidin-1- yl)pyrimidine-5-carboxamide |
| 162 | 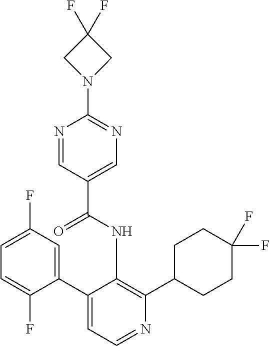 [SMILES: O=C(Nc1c(-c2cc(F)ccc2F)ccnc1C1CCC(F)(F)CC1)c1cnc(N2CC(F)(F)C2)nc1] | 2-(3,3-difluoroazetidin-1-yl)-N-(2- (4,4-difluorocyclohexyl)-4-(2,5- difluorophenyl)pyridin-3- yl)pyrimidine-5-carboxamide |
| 163 | 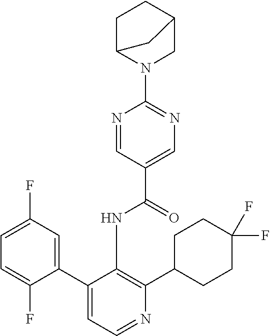 [SMILES: O=C(Nc1c(-c2cc(F)ccc2F)ccnc1C1CCC(F)(F)CC1)c1cnc(N2CC3CCC2C3)nc1] | 2-(2-azabicyclo[2.2.1]heptan-2- yl)-N-(2-(4,4-difluorocyclohexyl)- 4-(2,5-difluorophenyl)pyridin-3- yl)pyrimidine-5-carboxamide |
| 164 | 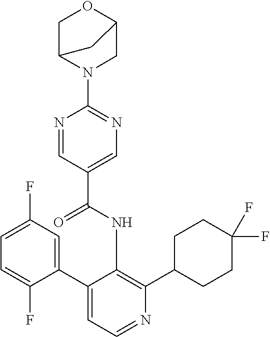 [SMILES: O=C(Nc1c(-c2cc(F)ccc2F)ccnc1C1CCC(F)(F)CC1)c1cnc(N2CC3CC2CO3)nc1] | 2-(2-oxa-5- azabicyclo[2.2.1]heptan-5-yl)-N- (2-(4,4-difluorocyclohexyl)-4- (2,5-difluorophenyl)pyridin-3- yl)pyrimidine-5-carboxamide |
| 165 | 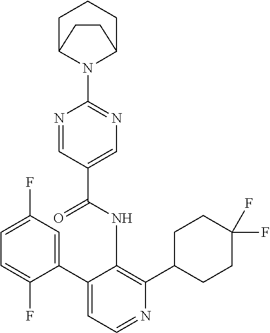 [SMILES: O=C(Nc1c(-c2cc(F)ccc2F)ccnc1C1CCC(F)(F)CC1)c1cnc(N2C3CCCC2CC3)nc1] | 2-(8-azabicyclo[3.2.1]octan-8-yl)- N-(2-(4,4-difluorocyclohexyl)-4- (2,5-difluorophenyl)pyridin-3- yl)pyrimidine-5-carboxamide |
| 166 | 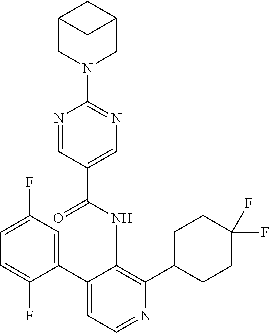 [SMILES: O=C(Nc1c(-c2cc(F)ccc2F)ccnc1C1CCC(F)(F)CC1)c1cnc(N2CC3CC(C3)C2)nc1] | 2-(3-azabicyclo[3.1.1]heptan-3- yl)-N-(2-(4,4-difluorocyclohexyl)- 4-(2,5-difluorophenyl)pyridin-3- yl)pyrimidine-5-carboxamide |
| 167 | 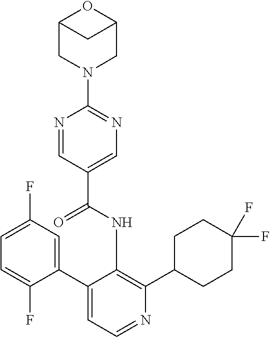 [SMILES: O=C(Nc1c(-c2cc(F)ccc2F)ccnc1C1CCC(F)(F)CC1)c1cnc(N2CC3CC(C2)O3)nc1] | 2-(6-oxa-3- azabicyclo[3.1.1]heptan-3-yl)-N- (2-(4,4-difluorocyclohexyl)-4- (2,5-difluorophenyl)pyridin-3- yl)pyrimidine-5-carboxamide |
| 168 | 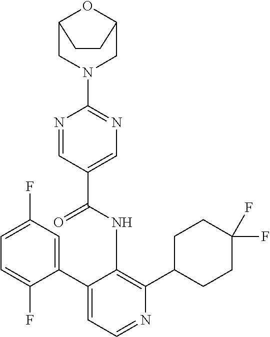 [SMILES: O=C(Nc1c(-c2cc(F)ccc2F)ccnc1C1CCC(F)(F)CC1)c1cnc(N2CC3CCC(C2)O3)nc1] | 2-(8-oxa-3- azabicyclo[3.2.1]octan-3-yl)-N- (2-(4,4-difluorocyclohexyl)-4- (2,5-difluorophenyl)pyridin-3- yl)pyrimidine-5-carboxamide |
| 169 | 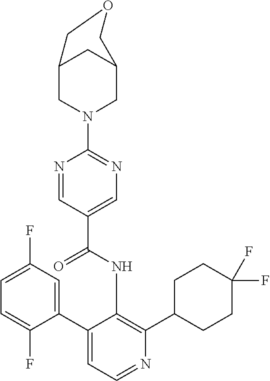 [SMILES: O=C(Nc1c(-c2cc(F)ccc2F)ccnc1C1CCC(F)(F)CC1)c1cnc(N2CC3COCC(C3)C2)nc1] | 2-(3-oxa-7- azabicyclo[3.3.1]nonan-7-yl)-N- (2-(4,4-difluorocyclohexyl)-4- (2,5-difluorophenyl)pyridin-3- yl)pyrimidine-5-carboxamide |
| 170 | 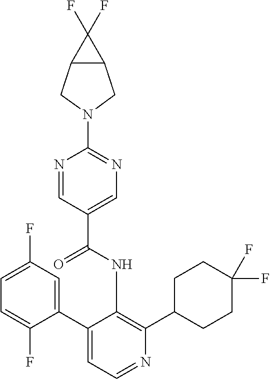 [SMILES: O=C(Nc1c(-c2cc(F)ccc2F)ccnc1C1CCC(F)(F)CC1)c1cnc(N2CC3C(C2)C3(F)F)nc1] | 2-(6,6-difluoro-3- azabicyclo[3.1.0]hexan-3-yl)-N- (2-(4,4-difluorocyclohexyl)-4- (2,5-difluorophenyl)pyridin-3- yl)pyrimidine-5-carboxamide |
| 171 | 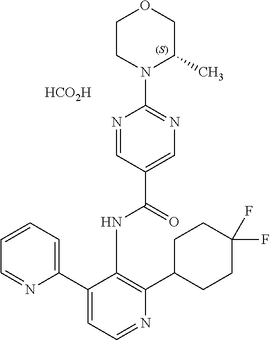 | (S)-N-(2′-(4,4- difluorocyclohexyl)-[2,4′- bipyridin]-3′-yl)-2-(3- methylmorpholino)pyrimidine-5- carboxamide formate salt |
| 172 | 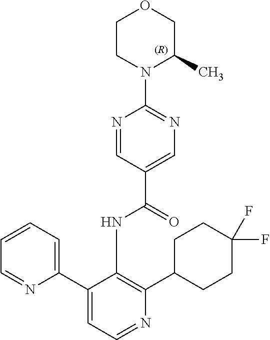 | (R)-N-(2′-(4,4- difluorocyclohexyl)-[2,4′- bipyridin]-3′-yl)-2-(3- methylmorpholino)pyrimidine-5- carboxamide |
| 173 | 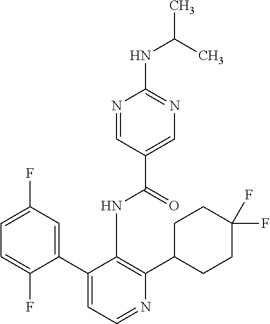 [SMILES: CC(C)Nc1ncc(C(=O)Nc2c(-c3cc(F)ccc3F)ccnc2C2CCC(F)(F)CC2)cn1] | N-(2-(4,4-difluorocyclohexyl)-4- (2,5-difluorophenyl)pyridin-3-yl)- 2-(isopropylamino)pyrimidine-5- carboxamide |
| 174 | 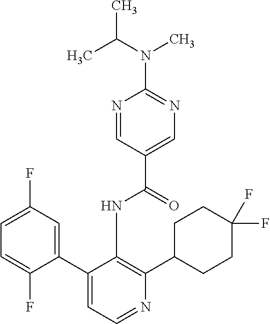 [SMILES: CC(C)N(C)c1ncc(C(=O)Nc2c(-c3cc(F)ccc3F)ccnc2C2CCC(F)(F)CC2)cn1] | N-(2-(4,4-difluorocyclohexyl)-4- (2,5-difluorophenyl)pyridin-3-yl)- 2-(isopropyl(methyl)amino) pyrimidine-5-carboxamide |
| 175 | 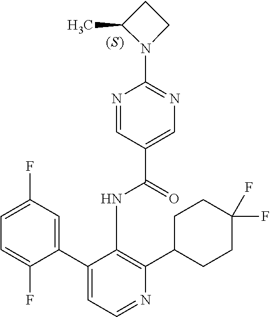 | (S)-N-(2-(4,4-difluorocyclohexyl)- 4-(2,5-difluorophenyl)pyridin-3- yl)-2-(2-methylazetidin-1- yl)pyrimidine-5-carboxamide |
| 176 | 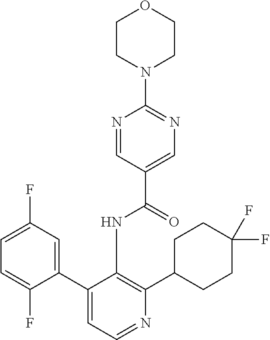 [SMILES: O=C(Nc1c(-c2cc(F)ccc2F)ccnc1C1CCC(F)(F)CC1)c1cnc(N2CCOCC2)nc1] | N-(2-(4,4-difluorocyclohexyl)-4- (2,5-difluorophenyl)pyridin-3-yl)- 2-morpholinopyrimidine-5- carboxamide |
| 177 | 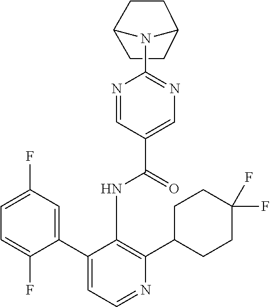 [SMILES: O=C(Nc1c(-c2cc(F)ccc2F)ccnc1C1CCC(F)(F)CC1)c1cnc(N2C3CCC2CC3)nc1] | 2-(7-azabicyclo[2.2.1]heptan-7- yl)-N-(2-(4,4-difluorocyclohexyl)- 4-(2,5-difluorophenyl)pyridin-3- yl)pyrimidine-5-carboxamide |
| 178 | 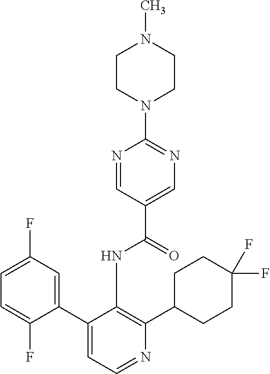 [SMILES: CN1CCN(c2ncc(C(=O)Nc3c(-c4cc(F)ccc4F)ccnc3C3CCC(F)(F)CC3)cn2)CC1] | N-(2-(4,4-difluorocyclohexyl)-4- (2,5-difluorophenyl)pyridin-3-yl)- 2-(4-methylpiperazin-1- yl)pyrimidine-5-carboxamide |
| 179 | 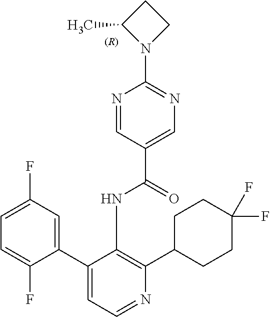 | (R)-N-(2-(4,4-difluorocyclohexyl)- 4-(2,5-difluorophenyl)pyridin-3- yl)-2-(2-methylazetidin-1- yl)pyrimidine-5-carboxamide |
| 180 | 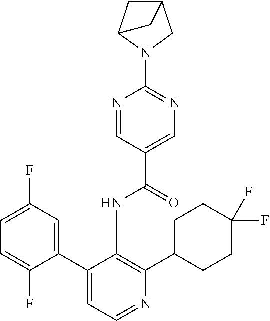 [SMILES: O=C(Nc1c(-c2cc(F)ccc2F)ccnc1C1CCC(F)(F)CC1)c1cnc(N2CC3CC2C3)nc1] | 2-(2-azabicyclo[2.1.1]hexan-2- yl)-N-(2-(4,4-difluorocyclohexyl)- 4-(2,5-difluorophenyl)pyridin-3- yl)pyrimidine-5-carboxamide |
| 181 | 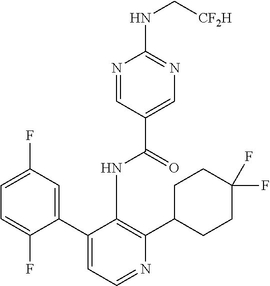 [SMILES: O=C(Nc1c(-c2cc(F)ccc2F)ccnc1C1CCC(F)(F)CC1)c1cnc(NCC(F)F)nc1] | N-(2-(4,4-difluorocyclohexyl)-4- (2,5-difluorophenyl)pyridin-3-yl)- 2-((2,2- difluoroethyl)amino)pyrimidine-5- carboxamide |
| 182 | 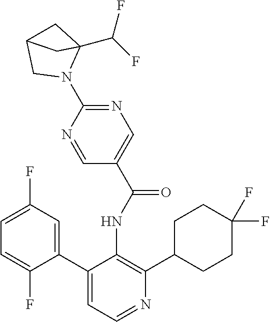 [SMILES: O=C(Nc1c(-c2cc(F)ccc2F)ccnc1C1CCC(F)(F)CC1)c1cnc(N2CC3CC2(C(F)F)C3)nc1] | N-(2-(4,4-difluorocyclohexyl)-4- (2,5-difluorophenyl)pyridin-3-yl)- 2-(1-(difluoromethyl)-2- azabicyclo[2.1.1]hexan-2- yl)pyrimidine-5-carboxamide |
| 183 | 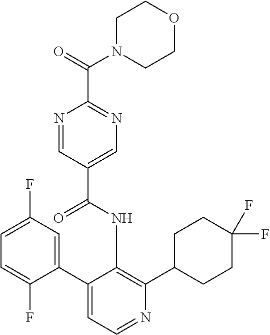 [SMILES: O=C(Nc1c(-c2cc(F)ccc2F)ccnc1C1CCC(F)(F)CC1)c1cnc(C(=O)N2CCOCC2)nc1] | N-(2-(4,4-difluorocyclohexyl)-4- (2,5-difluorophenyl)pyridin-3-yl)- 2-(morpholine-4- carbonyl)pyrimidine-5- carboxamide |
| 184 | 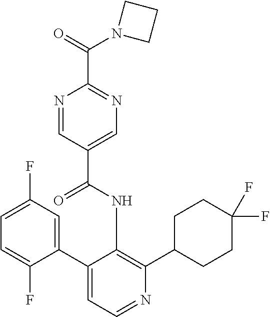 [SMILES: O=C(Nc1c(-c2cc(F)ccc2F)ccnc1C1CCC(F)(F)CC1)c1cnc(C(=O)N2CCC2)nc1] | 2-(azetidine-1-carbonyl)-N-(2- (4,4-difluorocyclohexyl)-4-(2,5- difluorophenyl)pyridin-3- yl)pyrimidine-5-carboxamide |
| 185 | 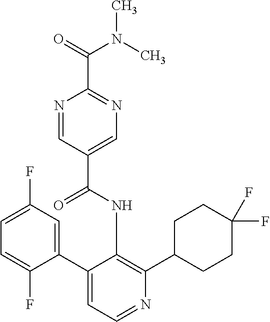 [SMILES: CN(C)C(=O)c1ncc(C(=O)Nc2c(-c3cc(F)ccc3F)ccnc2C2CCC(F)(F)CC2)cn1] | N⁵-[2-(4,4-difluorocyclohexyl)-4- (2,5-difluorophenyl)-3-pyridyl]- N²,N²-dimethyl-pyrimidine-2,5- dicarboxamide |
| 186 | 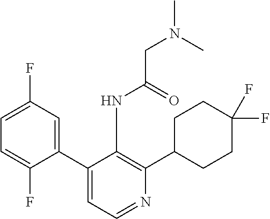 [SMILES: CN(C)CC(=O)Nc1c(-c2cc(F)ccc2F)ccnc1C1CCC(F)(F)CC1] | N-(2-(4,4-difluorocyclohexyl)-4- (2,5-difluorophenyl)pyridin-3-yl)- 2-(dimethylamino)acetamide |
| 187 | 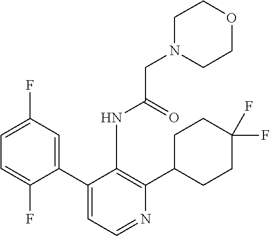 [SMILES: O=C(CN1CCOCC1)Nc1c(-c2cc(F)ccc2F)ccnc1C1CCC(F)(F)CC1] | N-(2-(4,4-difluorocyclohexyl)-4- (2,5-difluorophenyl)pyridin-3-yl)- 2-morpholinoacetamide |
| 188 | 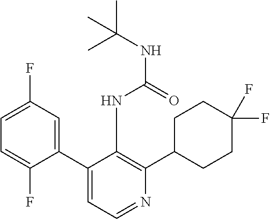 [SMILES: CC(C)(C)NC(=O)Nc1c(-c2cc(F)ccc2F)ccnc1C1CCC(F)(F)CC1] | 1-(tert-butyl)-3-(2-(4,4- difluorocyclohexyl)-4-(2,5- difluorophenyl)pyridin-3-yl)urea |
| 189 | 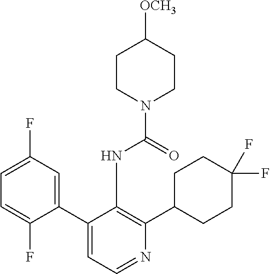 [SMILES: COC1CCN(C(=O)Nc2c(-c3cc(F)ccc3F)ccnc2C2CCC(F)(F)CC2)CC1] | N-(2-(4,4-difluorocyclohexyl)-4- (2,5-difluoro phenyl)pyridin-3-yl)- 4-methoxypiperidine-1- carboxamide |
| 190 | 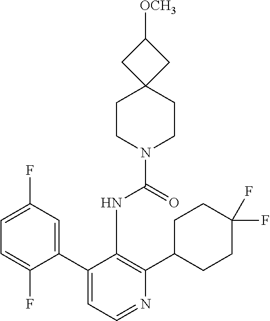 [SMILES: COC1CC2(CCN(C(=O)Nc3c(-c4cc(F)ccc4F)ccnc3C3CCC(F)(F)CC3)CC2)C1] | N-(2-(4,4-difluorocyclohexyl)-4- (2,5-difluorophenyl)pyridin-3-yl)- 2-methoxy-7- azaspiro[3.5]nonane-7- carboxamide |
| 191 | 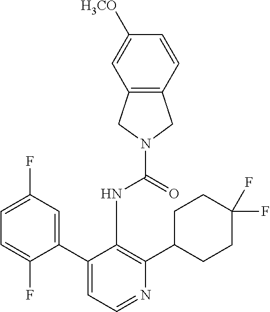 [SMILES: COc1ccc2c(c1)CN(C(=O)Nc1c(-c3cc(F)ccc3F)ccnc1C1CCC(F)(F)CC1)C2] | N-(2-(4,4-difluorocyclohexyl)-4- (2,5-difluorophenyl)pyridin-3-yl)- 5-methoxyisoindoline-2- carboxamide |
| 192 | 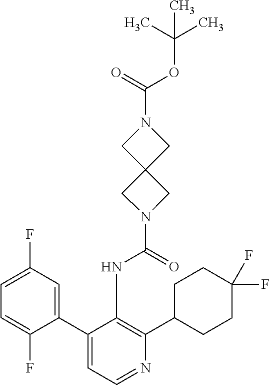 [SMILES: CC(C)(C)OC(=O)N1CC2(CN(C(=O)Nc3c(-c4cc(F)ccc4F)ccnc3C3CCC(F)(F)CC3)C2)C1] | tert-butyl 6-((2-(4,4- difluorocyclohexyl)-4-(2,5- difluorophenyl)pyridin-3- yl)carbamoyl)-2,6- diazaspiro[3.3]heptane-2- carboxylate |
| 193 | 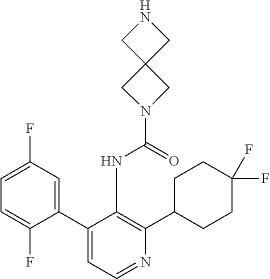 [SMILES: O=C(Nc1c(-c2cc(F)ccc2F)ccnc1C1CCC(F)(F)CC1)N1CC2(CNC2)C1] | N-(2-(4,4-difluorocyclohexyl)-4- (2,5-difluorophenyl)pyridin-3-yl)- 2,6-diazaspiro[3.3]heptane-2- carboxamide |
| 194 | 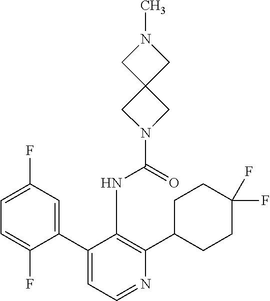 [SMILES: CN1CC2(C1)CN(C(=O)Nc1c(-c3cc(F)ccc3F)ccnc1C1CCC(F)(F)CC1)C2] | N-(2-(4,4-difluorocyclohexyl)-4- (2,5-difluorophenyl)pyridin-3-yl)- 6-methyl-2,6- diazaspiro[3.3]heptane-2- carboxamide |
| 195 | 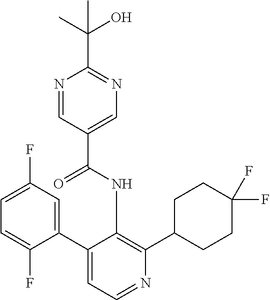 [SMILES: CC(C)(O)c1ncc(C(=O)Nc2c(-c3cc(F)ccc3F)ccnc2C2CCC(F)(F)CC2)cn1] | N-(2-(4,4-difluorocyclohexyl)-4- (2,5-difluorophenyl)pyridin-3-yl)- 2-(2-hydroxypropan-2- yl)pyrimidine-5-carboxamide |
| 196 | 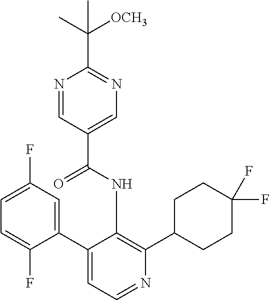 [SMILES: COC(C)(C)c1ncc(C(=O)Nc2c(-c3cc(F)ccc3F)ccnc2C2CCC(F)(F)CC2)cn1] | N-(2-(4,4-difluorocyclohexyl)-4- (2,5-difluorophenyl)pyridin-3-yl)- 2-(2-methoxypropan-2- yl)pyrimidine-5-carboxamide |
| 197 | 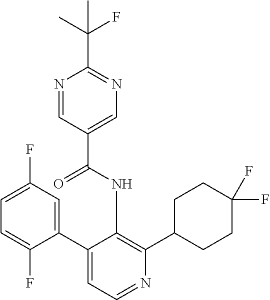 [SMILES: CC(C)(F)c1ncc(C(=O)Nc2c(-c3cc(F)ccc3F)ccnc2C2CCC(F)(F)CC2)cn1] | N-(2-(4,4-difluorocyclohexyl)-4- (2,5-difluorophenyl)pyridin-3-yl)- 2-(2-fluoropropan-2- yl)pyrimidine-5-carboxamide |
| 198 | 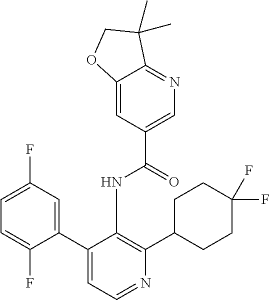 [SMILES: CC1(C)COc2cc(C(=O)Nc3c(-c4cc(F)ccc4F)ccnc3C3CCC(F)(F)CC3)cnc21] | N-(2-(4,4-difluorocyclohexyl)-4- (2,5-difluorophenyl)pyridin-3-yl)- 2-(2-fluoropropan-2- yl)pyrimidine-5-carboxamide |
| 199 | 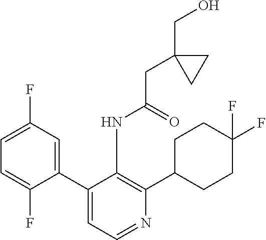 [SMILES: O=C(CC1(CO)CC1)Nc1c(-c2cc(F)ccc2F)ccnc1C1CCC(F)(F)CC1] | N-(2-(4,4-difluorocyclohexyl)-4- (2,5-difluorophenyl)pyridin-3-yl)- 2-(1-(hydroxymethyl)cyclopropyl) acetamide |
| 200 | 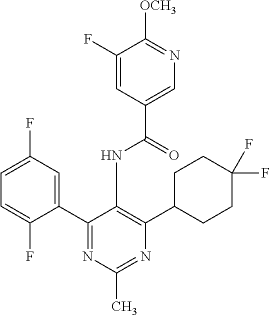 [SMILES: COc1ncc(C(=O)Nc2c(-c3cc(F)ccc3F)nc(C)nc2C2CCC(F)(F)CC2)cc1F] | N-(4-(4,4-difluorocyclohexyl)-6- (2,5-difluorophenyl)-2- methylpyrimidin-5-yl)-5-fluoro-6- methoxynicotinamide |
| 201 | 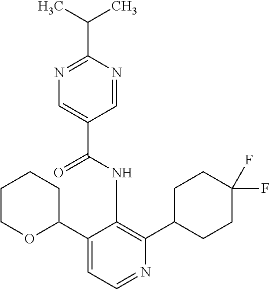 [SMILES: CC(C)c1ncc(C(=O)Nc2c(C3CCCCO3)ccnc2C2CCC(F)(F)CC2)cn1] | N-(2-(4,4-difluorocyclohexyl)-4- (tetrahydro-2H-pyran-2- yl)pyridin-3-yl)-2- isopropylpyrimidine-5- carboxamide |
| 202 | 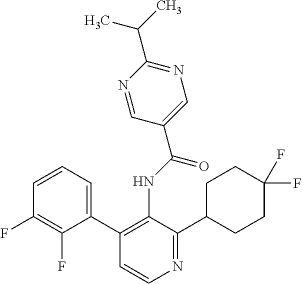 [SMILES: CC(C)c1ncc(C(=O)Nc2c(-c3cccc(F)c3F)ccnc2C2CCC(F)(F)CC2)cn1] | N-(2-(4,4-difluorocyclohexyl)-4- (2,3-difluorophenyl)pyridin-3-yl)- 2-isopropylpyrimidine-5- carboxamide |
| 203 | 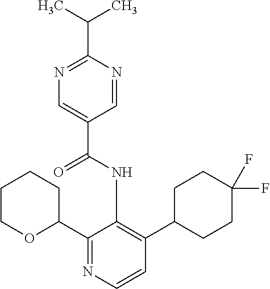 [SMILES: CC(C)c1ncc(C(=O)Nc2c(C3CCC(F)(F)CC3)ccnc2C2CCCCO2)cn1] | N-(2-(4,4-difluorocyclohexyl)-4- (tetrahydro-2H-pyran-2- yl)pyridin-3-yl)-2- isopropylpyrimidine-5- carboxamide |
| 204 | 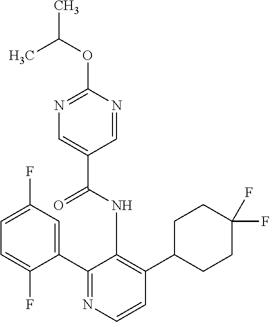 [SMILES: CC(C)Oc1ncc(C(=O)Nc2c(C3CCC(F)(F)CC3)ccnc2-c2cc(F)ccc2F)cn1] | N-(4-(4,4-difluorocyclohexyl)-2- (2,5-difluorophenyl)pyridin-3-yl)- 2-isopropoxypyrimidine-5- carboxamide |
| 205 | 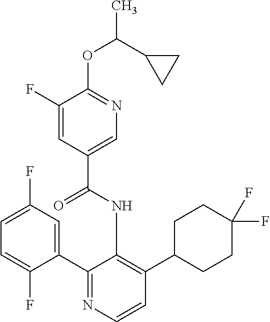 [SMILES: CC(Oc1ncc(C(=O)Nc2c(C3CCC(F)(F)CC3)ccnc2-c2cc(F)ccc2F)cc1F)C1CC1] | 6-(1-cyclopropylethoxy)-N-(4- (4,4-difluorocyclohexyl)-2-(2,5- difluorophenyl)pyridin-3-yl)-5- fluoronicotinamide |
| 206 | 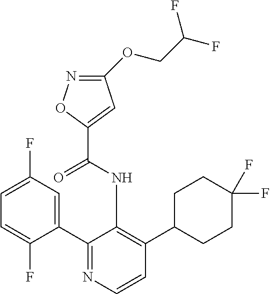 [SMILES: O=C(Nc1c(C2CCC(F)(F)CC2)ccnc1-c1cc(F)ccc1F)c1cc(OCC(F)F)no1] | N-(4-(4,4-difluorocyclohexyl)-2- phenylpyridin-3-yl)-2- isopropylpyrimidine-5- carboxamide |
| 207 | 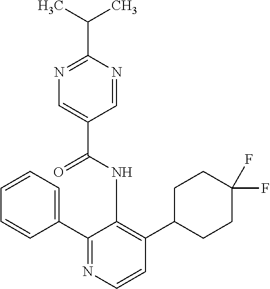 [SMILES: CC(C)c1ncc(C(=O)Nc2c(C3CCC(F)(F)CC3)ccnc2-c2ccccc2)cn1] | N-(4-(4,4-difluorocyclohexyl)-2- phenylpyridin-3-yl)-2- isopropylpyrimidine-5- carboxamide |
| 208 | 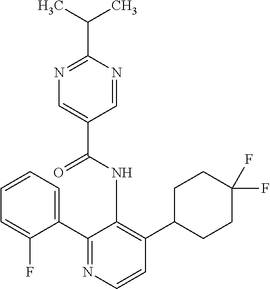 [SMILES: CC(C)c1ncc(C(=O)Nc2c(C3CCC(F)(F)CC3)ccnc2-c2ccccc2F)cn1] | N-(4-(4,4-difluorocyclohexyl)-2- (2-fluorophenyl)pyridin-3-yl)-2- isopropylpyrimidine-5- carboxamide |
| 209 | 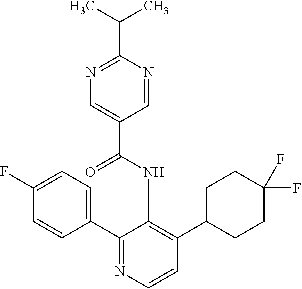 [SMILES: CC(C)c1ncc(C(=O)Nc2c(C3CCC(F)(F)CC3)ccnc2-c2ccc(F)cc2)cn1] | N-(4-(4,4-difluorocyclohexyl)-2- (4-fluorophenyl)pyridin-3-yl)-2- isopropylpyrimidine-5- carboxamide |
| 210 | 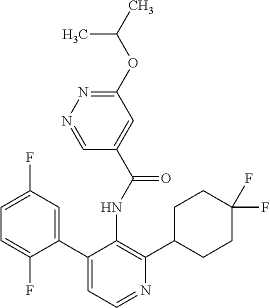 [SMILES: CC(C)Oc1cc(C(=O)Nc2c(-c3cc(F)ccc3F)ccnc2C2CCC(F)(F)CC2)cnn1] | N-(2-(4,4-difluorocyclohexyl)-4- (2,5-difluorophenyl)pyridin-3-yl)- 6-isopropoxypyridazine-4- carboxamide |
| 211 | 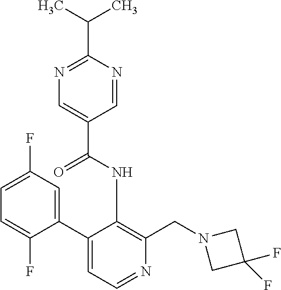 [SMILES: CC(C)c1ncc(C(=O)Nc2c(-c3cc(F)ccc3F)ccnc2CN2CC(F)(F)C2)cn1] | N-(2-((3,3-difluoroazetidin-1- yl)methyl)-4-(2,5- difluorophenyl)pyridin-3-yl)-2- isopropylpyrimidine-5- carboxamide |
| 212 | 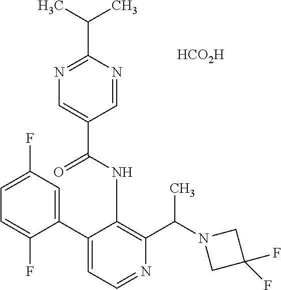 [SMILES: CC(C)c1ncc(C(=O)Nc2c(-c3cc(F)ccc3F)ccnc2C(C)N2CC(F)(F)C2)cn1 ; "HCO2H"] | N-(2-(1-(3,3-difluoroazetidin-1- yl) ethyl)-4-(2,5- difluorophenyl)pyridin-3-yl)-2- isopropylpyrimidine-5- carboxamide formate salt P1 |
| 213 | 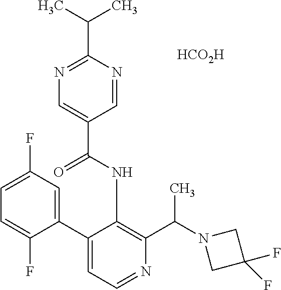 [SMILES: CC(C)c1ncc(C(=O)Nc2c(-c3cc(F)ccc3F)ccnc2C(C)N2CC(F)(F)C2)cn1 ; "HCO2H"] | N-(2-(1-(3,3-difluoroazetidin-1- yl) ethyl)-4-(2,5- difluorophenyl)pyridin-3-yl)-2- isopropylpyrimidine-5- carboxamide formate salt P2 |
| 214 | 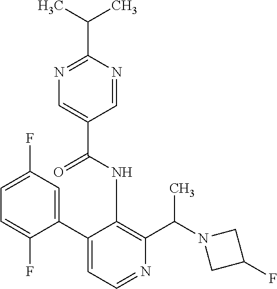 [SMILES: CC(C)c1ncc(C(=O)Nc2c(-c3cc(F)ccc3F)ccnc2C(C)N2CC(F)C2)cn1] | N-(4-(2,5-difluorophenyl)-2-(1- (3-fluoroazetidin-1- yl)ethyl)pyridin-3-yl)-2- isopropylpyrimidine-5- carboxamide P1 |
| 215 | 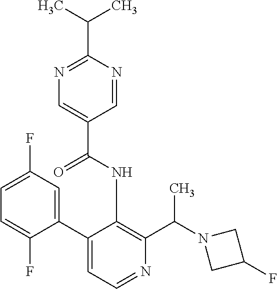 [SMILES: CC(C)c1ncc(C(=O)Nc2c(-c3cc(F)ccc3F)ccnc2C(C)N2CC(F)C2)cn1] | N-(4-(2,5-difluorophenyl)-2-(1-(3- fluoroazetidin-1-yl)ethyl)pyridin- 3-yl)-2-isopropylpyrimidine-5- carboxamide P2 |
| 216 | 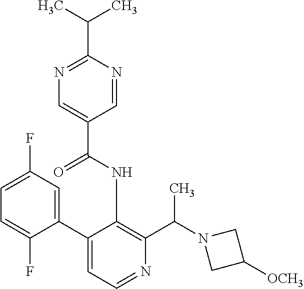 [SMILES: COC1CN(C(C)c2nccc(-c3cc(F)ccc3F)c2NC(=O)c2cnc(C(C)C)nc2)C1] | N-(4-(2,5-difluorophenyl)-2-(1- (3-methoxyazetidin-1- yl)ethyl)pyridin-3-yl)-2- isopropylpyrimidine-5- carboxamide P1 |
| 217 | 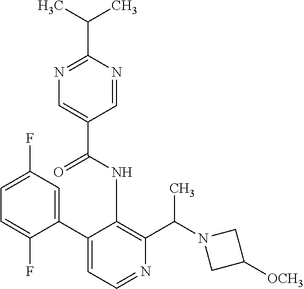 [SMILES: COC1CN(C(C)c2nccc(-c3cc(F)ccc3F)c2NC(=O)c2cnc(C(C)C)nc2)C1] | N-(4-(2,5-difluorophenyl)-2-(1-(3- methoxyazetidin-1- yl)ethyl)pyridin-3-yl)-2- isopropylpyrimidine-5- carboxamide P2 |
| 218 | 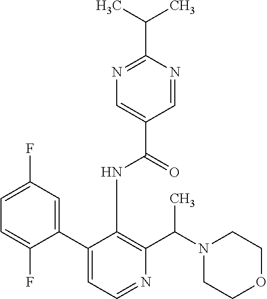 [SMILES: CC(C)c1ncc(C(=O)Nc2c(-c3cc(F)ccc3F)ccnc2C(C)N2CCOCC2)cn1] | N-(4-(2,5-difluorophenyl)-2-(1- morpholinoethyl)pyridin-3-yl)-2- isopropylpyrimidine-5- carboxamide P1 |
| 219 | 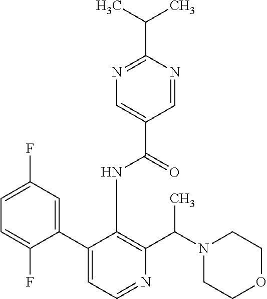 [SMILES: CC(C)c1ncc(C(=O)Nc2c(-c3cc(F)ccc3F)ccnc2C(C)N2CCOCC2)cn1] | N-(4-(2,5-difluorophenyl)-2-(1-(3- methoxyazetidin-1- yl)ethyl)pyridin-3-yl)-2- isopropylpyrimidine-5- carboxamide P2 |
| 220 | 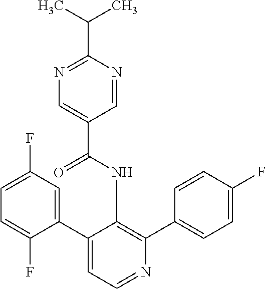 [SMILES: CC(C)c1ncc(C(=O)Nc2c(-c3cc(F)ccc3F)ccnc2-c2ccc(F)cc2)cn1] | N-(4-(2,5-difluorophenyl)-2-(4- fluorophenyl)pyridin-3-yl)-2- isopropylpyrimidine-5- carboxamide |
| 221 | 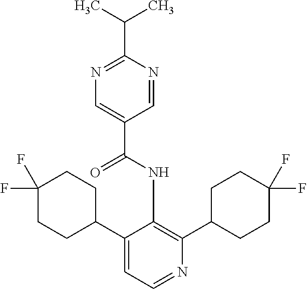 [SMILES: CC(C)c1ncc(C(=O)Nc2c(C3CCC(F)(F)CC3)ccnc2C2CCC(F)(F)CC2)cn1] | N-(2,4-bis(4,4- difluorocyclohexyl)pyridin-3-yl)- 2-isopropylpyrimidine-5- carboxamide |
| 222 | 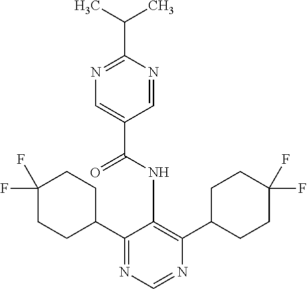 [SMILES: CC(C)c1ncc(C(=O)Nc2c(C3CCC(F)(F)CC3)ncnc2C2CCC(F)(F)CC2)cn1] | N-(4,6-bis(4,4- difluorocyclohexyl)-pyrimidin-5- yl)-2-isopropylpyrimidine-5- carboxamide |
| 223 | 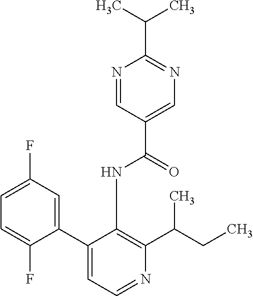 [SMILES: CCC(C)c1nccc(-c2cc(F)ccc2F)c1NC(=O)c1cnc(C(C)C)nc1] | N-(2-(sec-butyl)-4-(2,5- difluorophenyl) pyridin-3-yl)-2- isopropylpyrimidine-5- carboxamide |
| 224 | 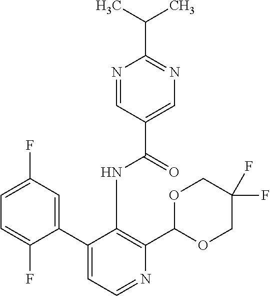 [SMILES: CC(C)c1ncc(C(=O)Nc2c(-c3cc(F)ccc3F)ccnc2C2OCC(F)(F)CO2)cn1] | N-(2-(5,5-difluoro-1,3-dioxan-2- yl)-4-(2,5-difluorophenyl)pyridin- 3-yl)-2-isopropylpyrimidine-5- carboxamide |
| 225 | 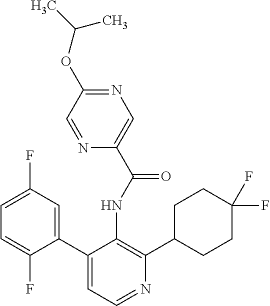 [SMILES: CC(C)Oc1cnc(C(=O)Nc2c(-c3cc(F)ccc3F)ccnc2C2CCC(F)(F)CC2)cn1] | N-(2-(4,4-difluorocyclohexyl)-4- (2,5-difluorophenyl)pyridin-3-yl)- 5-isopropoxypyrazine-2- carboxamide |
| 226 | 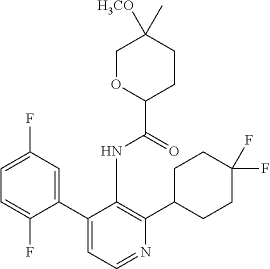 [SMILES: COC1(C)CCC(C(=O)Nc2c(-c3cc(F)ccc3F)ccnc2C2CCC(F)(F)CC2)OC1] | trans-N-(2-(4,4- difluorocyclohexyl)-4-(2,5- difluorophenyl)pyridin-3-yl)-5- methoxy-5-methyltetrahydro-2H- pyran-2-carboxamide P1 |
| 227 | 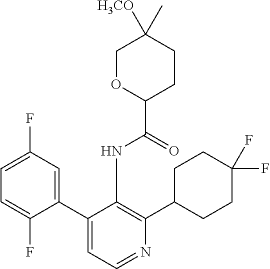 [SMILES: COC1(C)CCC(C(=O)Nc2c(-c3cc(F)ccc3F)ccnc2C2CCC(F)(F)CC2)OC1] | trans-N-(2-(4,4- difluorocyclohexyl)-4-(2,5- difluorophenyl)pyridin-3-yl)-5- methoxy-5-methyltetrahydro-2H- pyran-2-carboxamide P2 |
| 228 | 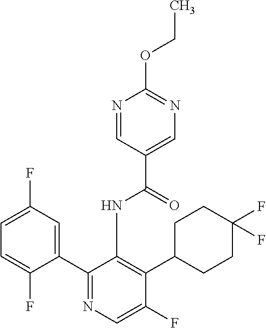 [SMILES: CCOc1ncc(C(=O)Nc2c(-c3cc(F)ccc3F)ncc(F)c2C2CCC(F)(F)CC2)cn1] | N-(4-(4,4-difluorocyclohexyl)-2- (2,5-difluorophenyl)-5- fluoropyridin-3-yl)-2- ethoxypyrimidine-5-carboxamide |
| 229 | 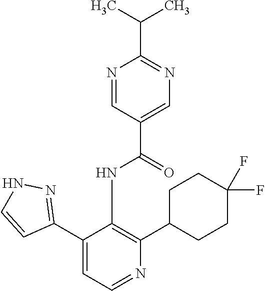 [SMILES: CC(C)c1ncc(C(=O)Nc2c(-c3cc[nH]n3)ccnc2C2CCC(F)(F)CC2)cn1] | N-(2-(4,4-difluorocyclohexyl)-4- (1H-pyrazol-3-yl)pyridin-3-yl)-2- isopropylpyrimidine-5- carboxamide |
| 230 | 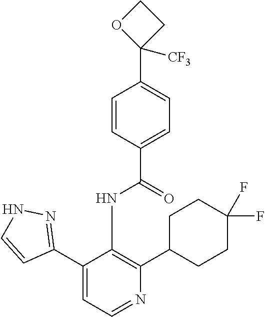 [SMILES: O=C(Nc1c(-c2cc[nH]n2)ccnc1C1CCC(F)(F)CC1)c1ccc(C2(C(F)(F)F)CCO2)cc1] | N-(2-(4,4-difluorocyclohexyl)-4- (1H-pyrazol-3-yl)pyridin-3-yl)-4- (2-(trifluoromethyl)oxetan-2- yl)benzamide |
| 231 | 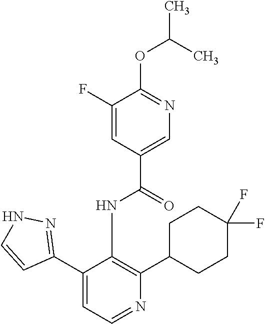 [SMILES: CC(C)Oc1ncc(C(=O)Nc2c(-c3cc[nH]n3)ccnc2C2CCC(F)(F)CC2)cc1F] | N-(2-(4,4-difluorocyclohexyl)-4- (1H-pyrazol-3-yl)pyridin-3-yl)-2- isopropylpyrimidine-5- carboxamide |
| 232 | 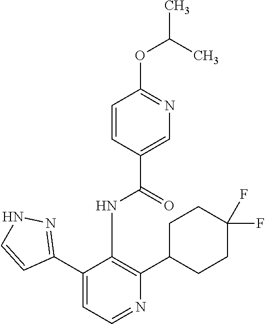 [SMILES: CC(C)Oc1ccc(C(=O)Nc2c(-c3cc[nH]n3)ccnc2C2CCC(F)(F)CC2)cn1] | N-(2-(4,4-difluorocyclohexyl)-4- (1H-pyrazol-3-yl)pyridin-3-yl)-6- isopropoxynicotinamide |
| 233 | 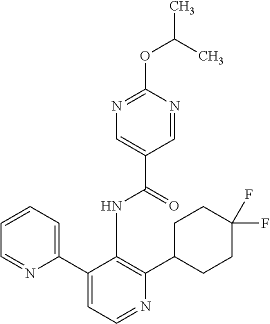 [SMILES: CC(C)Oc1ncc(C(=O)Nc2c(-c3ccccn3)ccnc2C2CCC(F)(F)CC2)cn1] | N-(2-(4,4-difluorocyclohexyl)-4- (1H-pyrazol-3-yl)pyridin-3-yl)-2- isopropylpyrimidine-5- carboxamide |
| 234 | 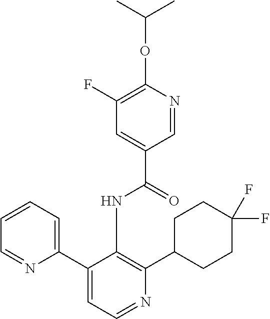 [SMILES: CC(C)Oc1ncc(C(=O)Nc2c(-c3ccccn3)ccnc2C2CCC(F)(F)CC2)cc1F] | N-(2′-(4,4-difluorocyclohexyl)- [2,4′-bipyridin]-3′-yl)-5-fluoro-6- isopropoxynicotinamide |
| 235 | 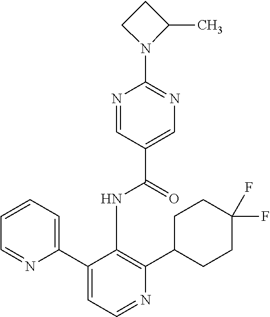 [SMILES: CC1CCN1c1ncc(C(=O)Nc2c(-c3ccccn3)ccnc2C2CCC(F)(F)CC2)cn1] | N-(2′-(4,4-difluorocyclohexyl)- [2,4′-bipyridin]-3′-yl)-2-(2- methylazetidin-1-yl)pyrimidine-5- carboxamide P1 |
| 236 | 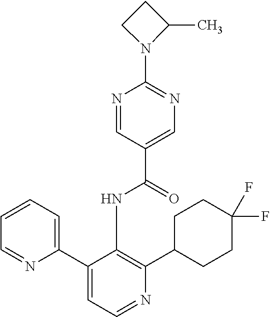 [SMILES: CC1CCN1c1ncc(C(=O)Nc2c(-c3ccccn3)ccnc2C2CCC(F)(F)CC2)cn1] | N-(2′-(4,4-difluorocyclohexyl)- [2,4′-bipyridin]-3′-yl)-2-(2- methylazetidin-1-yl)pyrimidine-5- carboxamide P2 |
| 237 | 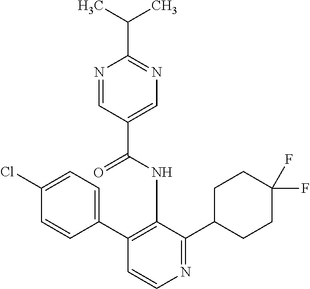 [SMILES: CC(C)c1ncc(C(=O)Nc2c(-c3ccc(Cl)cc3)ccnc2C2CCC(F)(F)CC2)cn1] | N-(4-(4-chlorophenyl)-2-(4,4- difluorocyclo hexyl)pyridin-3-yl)- 2-isopropylpyrimidine-5- carboxamide |
| 238 | 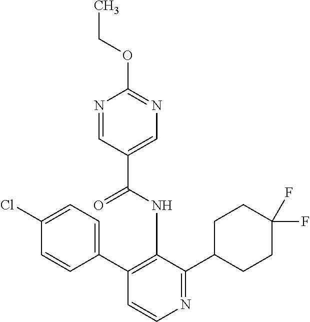 [SMILES: CCOc1ncc(C(=O)Nc2c(-c3ccc(Cl)cc3)ccnc2C2CCC(F)(F)CC2)cn1] | N-(4-(4-chloropheny)-2-(4,4- difluorocyclo hexyl)pyridin-3-yl)- 2-isopropylpyrimidine-5- carboxamide |
| 239 | 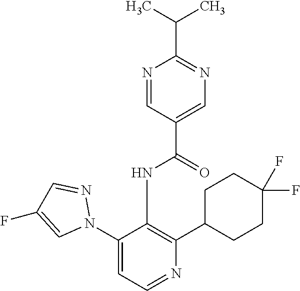 [SMILES: CC(C)c1ncc(C(=O)Nc2c(-n3cc(F)cn3)ccnc2C2CCC(F)(F)CC2)cn1] | N-(2-(4,4-difluorocyclohexyl)-4- (4-fluoro-1H-pyrazol-1-yl)pyridin- 3-yl)-2-isopropylpyrimidine-5- carboxamide |
| 240 | 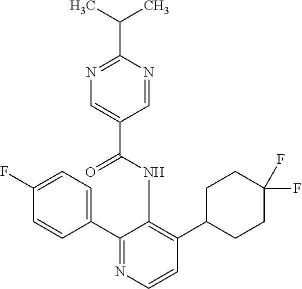 [SMILES: CC(C)c1ncc(C(=O)Nc2c(C3CCC(F)(F)CC3)ccnc2-c2ccc(F)cc2)cn1] | N-(4-(4,4-difluorocyclohexyl)-2- (4-fluorophenyl)pyridin-3-yl)-2- isopropylpyrimidine-5- carboxamide |
| 241 | 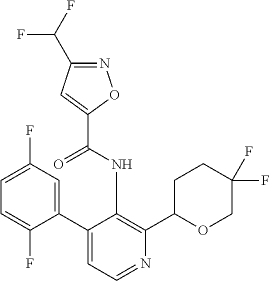 [SMILES: O=C(Nc1c(-c2cc(F)ccc2F)ccnc1C1CCC(F)(F)CO1)c1cc(C(F)F)no1] | rac-3-(difluoromethyl)-N-(4-(2,5- difluorophenyl)-2-(5,5- difluorotetrahydro-2H-pyran-2- yl)pyridin-3-yl)isoxazole-5- carboxamide |
| 242 | 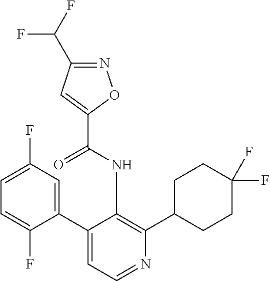 [SMILES: O=C(Nc1c(-c2cc(F)ccc2F)ccnc1C1CCC(F)(F)CC1)c1cc(C(F)F)no1] | N-(2-(4,4-difluorocyclohexyl)-4- (2,5-difluorophenyl)pyridin-3-yl)- 3-(difluoromethyl)isoxazole-5- carboxamide |
| 243 | 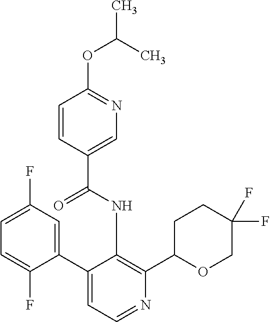 [SMILES: CC(C)Oc1ccc(C(=O)Nc2c(-c3cc(F)ccc3F)ccnc2C2CCC(F)(F)CO2)cn1] | rac-N-(4-(2,5-difluorophenyl)-2- (5,5-difluorotetrahydro-2H-pyran- 2-yl)pyridin-3-yl)-6- isopropoxynicotinamide |
| 244 | 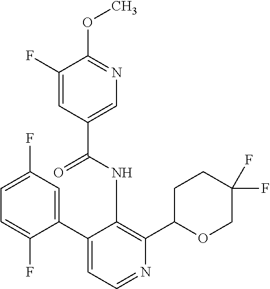 [SMILES: COc1ncc(C(=O)Nc2c(-c3cc(F)ccc3F)ccnc2C2CCC(F)(F)CO2)cc1F] | rac-N-(4-(2,5-difluorophenyl)-2- (5,5-difluorotetrahydro-2H-pyran- 2-yl)pyridin-3-yl)-5-fluoro-6- methoxynicotinamide |
| 245 | 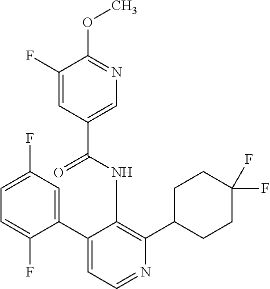 [SMILES: COc1ncc(C(=O)Nc2c(-c3cc(F)ccc3F)ccnc2C2CCC(F)(F)CC2)cc1F] | N-(2-(4,4-difluorocyclohexyl)-4- (2,5-difluorophenyl)pyridin-3-yl)- 5-fluoro-6-methoxynicotinamide |
| 246 | 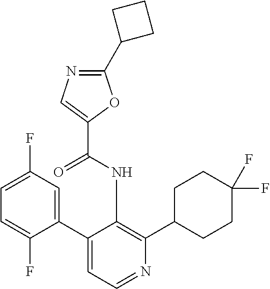 [SMILES: O=C(Nc1c(-c2cc(F)ccc2F)ccnc1C1CCC(F)(F)CC1)c1cnc(C2CCC2)o1] | 2-cyclobutyl-N-(2-(4,4- difluorocyclohexyl)-4-(2,5- difluorophenyl)pyridin-3- yl)oxazole-5-carboxamide |
| 247 | 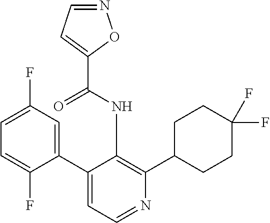 [SMILES: O=C(Nc1c(-c2cc(F)ccc2F)ccnc1C1CCC(F)(F)CC1)c1ccno1] | N-(2-(4,4-difluorocyclohexyl)-4- (2,5-difluorophenyl)pyridin-3- yl)isoxazole-5-carboxamide |
| 248 | 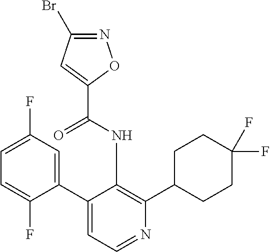 [SMILES: O=C(Nc1c(-c2cc(F)ccc2F)ccnc1C1CCC(F)(F)CC1)c1cc(Br)no1] | 3-bromo-N-(2-(4,4- difluorocyclohexyl)-4-(2,5- difluorophenyl)pyridin-3- yl)isoxazole-5-carboxamide |
| 249 | 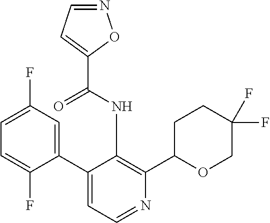 [SMILES: O=C(Nc1c(-c2cc(F)ccc2F)ccnc1C1CCC(F)(F)CO1)c1ccno1] | rac-N-(4-(2,5-difluorophenyl)-2- (5,5-difluorotetrahydro-2H-pyran- 2-yl)pyridin-3-yl)isoxazole-5- carboxamide |
| 250 | 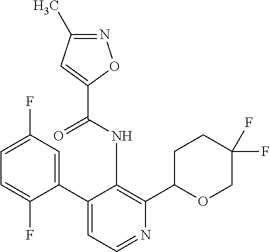 [SMILES: Cc1cc(C(=O)Nc2c(-c3cc(F)ccc3F)ccnc2C2CCC(F)(F)CO2)on1] | rac-N-(4-(2,5-difluorophenyl)-2- (5,5-difluorotetrahydro-2H-pyran- 2-yl)pyridin-3-yl)-3- methylisoxazole-5-carboxamide |
| 251 | 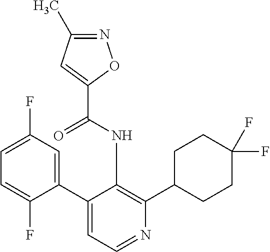 [SMILES: Cc1cc(C(=O)Nc2c(-c3cc(F)ccc3F)ccnc2C2CCC(F)(F)CC2)on1] | N-(2-(4,4-difluorocyclohexyl)-4- (2,5-difluorophenyl)pyridin-3-yl)- 3-methylisoxazole-5- carboxamide |
| 252 | 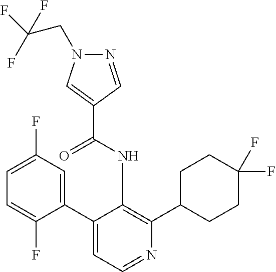 [SMILES: O=C(Nc1c(-c2cc(F)ccc2F)ccnc1C1CCC(F)(F)CC1)c1cnn(CC(F)(F)F)c1] | N-(2-(4,4-difluorocyclohexyl)-4- (2,5-difluorophenyl)pyridin-3-yl)- 1-(2,2,2-trifluoroethyl)-1H- pyrazole-4-carboxamide |
| 253 | 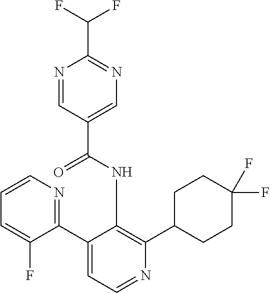 [SMILES: O=C(Nc1c(-c2ncccc2F)ccnc1C1CCC(F)(F)CC1)c1cnc(C(F)F)nc1] | N-(2′-(4,4-difluorocyclohexyl)-3- fluoro-[2,4′-bipyridin]-3′-yl)-2- (difluoromethyl)pyrimidine-5- carboxamide |
| 254 | 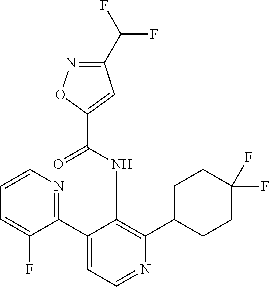 [SMILES: O=C(Nc1c(-c2ncccc2F)ccnc1C1CCC(F)(F)CC1)c1cc(C(F)F)no1] | N-(2′-(4,4-difluorocyclohexyl)-3- fluoro-[2,4′-bipyridin]-3′-yl)-3- (difluoromethyl)isoxazole-5- carboxamide |
| 255 | 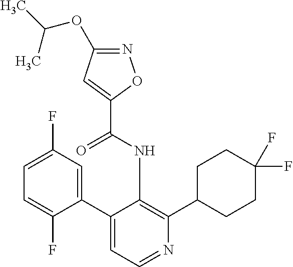 [SMILES: CC(C)Oc1cc(C(=O)Nc2c(-c3cc(F)ccc3F)ccnc2C2CCC(F)(F)CC2)on1] | N-(2-(4,4-difluorocyclohexyl)-4- (2,5-difluorophenyl)pyridin-3-yl)- 3-isopropylisoxazole-5- carboxamide |
| 256 | 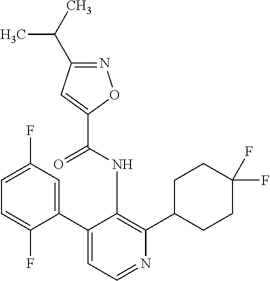 [SMILES: CC(C)c1cc(C(=O)Nc2c(-c3cc(F)ccc3F)ccnc2C2CCC(F)(F)CC2)on1] | N-(2-(4,4-difluorocyclohexyl)-4- (2,5-difluorophenyl)pyridin-3-yl)- 3-isopropoxyisoxazole-5- carboxamide |
| 257 | 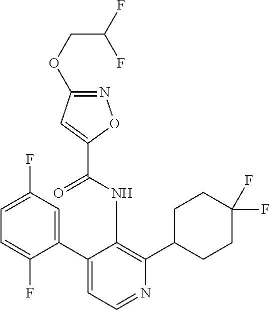 [SMILES: O=C(Nc1c(-c2cc(F)ccc2F)ccnc1C1CCC(F)(F)CC1)c1cc(OCC(F)F)no1] | N-(2-(4,4-difluorocyclohexyl)-4- (2,5-difluorophenyl)pyridin-3-yl)- 3-(2,2-difluoroethoxy)isoxazole- 5-carboxamide |
| 258 | 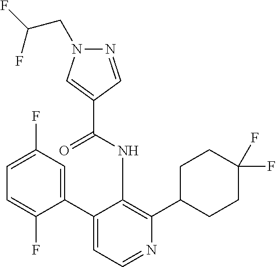 [SMILES: O=C(Nc1c(-c2cc(F)ccc2F)ccnc1C1CCC(F)(F)CC1)c1cnn(CC(F)F)c1] | N-(2-(4,4-difluorocyclohexyl)-4- (2,5-difluorophenyl)pyridin-3-yl)- 1-(2,2-difluoroethyl)-1H- pyrazole-4-carboxamide |
| 259 | 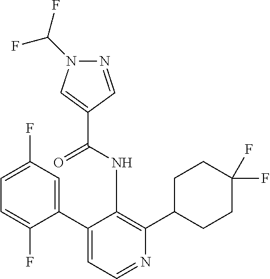 [SMILES: O=C(Nc1c(-c2cc(F)ccc2F)ccnc1C1CCC(F)(F)CC1)c1cnn(C(F)F)c1] | N-(2-(4,4-difluorocyclohexyl)-4- (2,5-difluorophenyl)pyridin-3-yl)- 1-(difluoromethyl)-1H-pyrazole- 4-carboxamide |
| 260 | 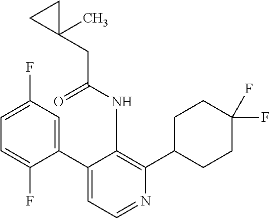 [SMILES: CC1(CC(=O)Nc2c(-c3cc(F)ccc3F)ccnc2C2CCC(F)(F)CC2)CC1] | N-(2-(4,4-difluorocyclohexyl)-4- (2,5-difluorophenyl)pyridin-3-yl)- 2-(1-methylcyclopropyl)acetamide |
| 261 | 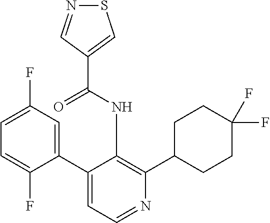 [SMILES: O=C(Nc1c(-c2cc(F)ccc2F)ccnc1C1CCC(F)(F)CC1)c1cnsc1] | N-(2-(4,4-difluorocyclohexyl)-4- (2,5-difluorophenyl)pyridin-3- yl)isothiazole-4-carboxamide |
| 262 | 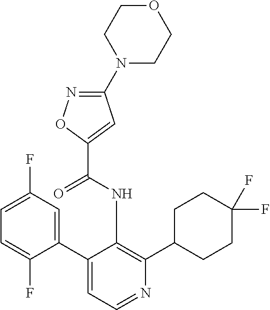 [SMILES: O=C(Nc1c(-c2cc(F)ccc2F)ccnc1C1CCC(F)(F)CC1)c1cc(N2CCOCC2)no1] | N-(2-(4,4-difluorocyclohexyl)-4- (2,5-difluorophenyl)pyridin-3-yl)- 3-morpholinoisoxazole-5- carboxamide |
| 263 | 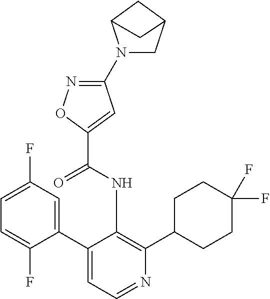 [SMILES: O=C(Nc1c(-c2cc(F)ccc2F)ccnc1C1CCC(F)(F)CC1)c1cc(N2CC3CC2C3)no1] | 3-(2-azabicyclo[2.1.1]hexan-2- yl)-N-(2-(4,4-difluorocyclohexyl)- 4-(2,5-difluorophenyl)pyridin-3- yl)isoxazole-5-carboxamide |
| 264 | 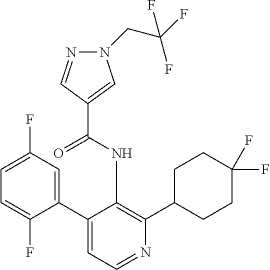 [SMILES: O=C(Nc1c(-c2cc(F)ccc2F)ccnc1C1CCC(F)(F)CC1)c1cnn(CC(F)(F)F)c1] | N-(2-(4,4-difluorocyclohexyl)-4- (2,5-difluorophenyl)pyridin-3-yl)- 1-(2,2,2-trifluoroethyl)-1H- pyrazole-4-carboxamide |
| 265 | 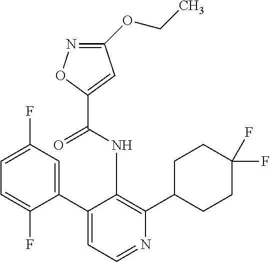 [SMILES: CCOc1cc(C(=O)Nc2c(-c3cc(F)ccc3F)ccnc2C2CCC(F)(F)CC2)on1] | N-(2-(4,4-difluorocyclohexyl)-4- (2,5-difluorophenyl)pyridin-3-yl)- 3-ethoxyisoxazole-5- carboxamide |
| 266 | 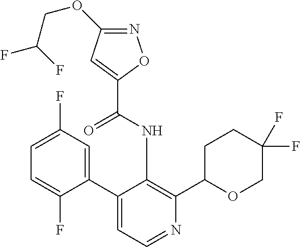 [SMILES: O=C(Nc1c(-c2cc(F)ccc2F)ccnc1C1CCC(F)(F)CO1)c1cc(OCC(F)F)no1] | rac-3-(2,2-difluoroethoxy)-N-(4- (2,5-difluorophenyl)-2-(5,5- difluorotetrahydro-2H-pyran-2- yl)pyridin-3-yl)isoxazole-5- carboxamide |
| 267 | 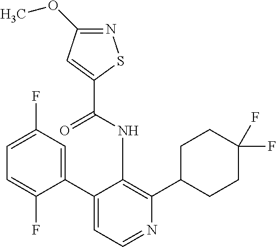 [SMILES: COc1cc(C(=O)Nc2c(-c3cc(F)ccc3F)ccnc2C2CCC(F)(F)CC2)sn1] | N-(2-(4,4-difluorocyclohexyl)-4- (2,5-difluorophenyl)pyridin-3-yl)- 3-methoxyisothiazole-5- carboxamide |
| 268 | 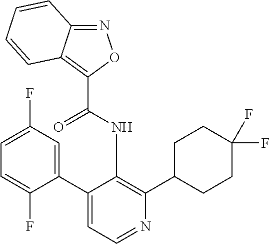 [SMILES: O=C(Nc1c(-c2cc(F)ccc2F)ccnc1C1CCC(F)(F)CC1)c1onc2ccccc12] | N-(2-(4,4-difluorocyclohexyl)-4- (2,5-difluorophenyl)pyridin-3- yl)benzo[c]isoxazole-3- carboxamide |
| 269 | 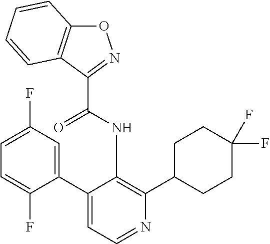 [SMILES: O=C(Nc1c(-c2cc(F)ccc2F)ccnc1C1CCC(F)(F)CC1)c1noc2ccccc12] | N-(2-(4,4-difluorocyclohexyl)-4- (2,5-difluorophenyl)pyridin-3- yl)benzo[d]isoxazole-3- carboxamide |
| 270 | 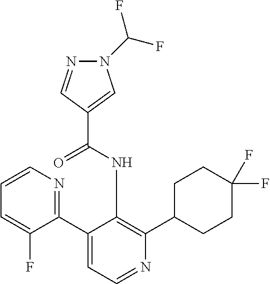 [SMILES: O=C(Nc1c(-c2ncccc2F)ccnc1C1CCC(F)(F)CC1)c1cnn(C(F)F)c1] | N-(2′-(4,4-difluorocyclohexyl)-3- fluoro-[2,4′-bipyridin]-3′-yl)-1- (difluoromethyl)-1H-pyrazole-4- carboxamide |
| 271 | 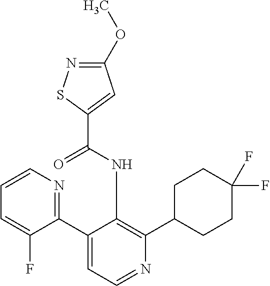 [SMILES: COc1cc(C(=O)Nc2c(-c3ncccc3F)ccnc2C2CCC(F)(F)CC2)sn1] | N-(2′-(4,4-difluorocyclohexyl)-3- fluoro-[2,4′-bipyridin]-3′-yl)-3- methoxyisothiazole-5- carboxamide |
| 272 | 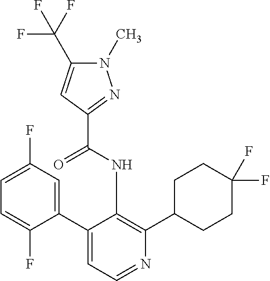 [SMILES: Cn1nc(C(=O)Nc2c(-c3cc(F)ccc3F)ccnc2C2CCC(F)(F)CC2)cc1C(F)(F)F] | N-(2-(4,4-difluorocyclohexyl)-4- (2,5-difluorophenyl)pyridin-3-yl)- 1-methyl-5-(trifluoromethyl)-1H- pyrazole-3-carboxamide |
| 273 | 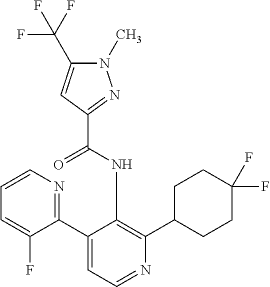 [SMILES: Cn1nc(C(=O)Nc2c(-c3ncccc3F)ccnc2C2CCC(F)(F)CC2)cc1C(F)(F)F] | N-(2′-(4,4-difluorocyclohexyl)-3- fluoro-[2,4′-bipyridin]-3′-yl)-1- methyl-5-(trifluoromethyl)-1H- pyrazole-3-carboxamide |
| 274 | 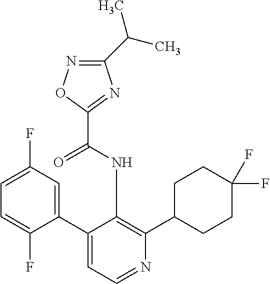 [SMILES: CC(C)c1noc(C(=O)Nc2c(-c3cc(F)ccc3F)ccnc2C2CCC(F)(F)CC2)n1] | N-(2-(4,4-difluorocyclohexyl)-4- (2,5-difluorophenyl)pyridin-3-yl)- 3-isopropyl-1,2,4-oxadiazole-5- carboxamide |
| 275 | 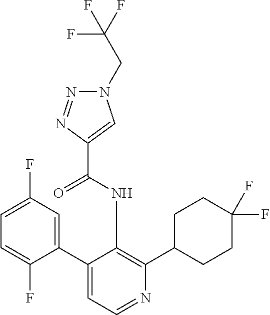 [SMILES: O=C(Nc1c(-c2cc(F)ccc2F)ccnc1C1CCC(F)(F)CC1)c1cn(CC(F)(F)F)nn1] | N-(2-(4,4-difluorocyclohexyl)-4- (2,5-difluorophenyl)pyridin-3-yl)- 1-(2,2,2-trifluoroethyl)-1H-1,2,3- triazole-4-carboxamide |
| 276 | 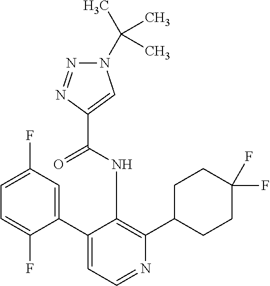 [SMILES: CC(C)(C)n1cc(C(=O)Nc2c(-c3cc(F)ccc3F)ccnc2C2CCC(F)(F)CC2)nn1] | 1-(tert-butyl)-N-(2-(4,4- difluorocyclohexyl)-4-(2,5- difluorophenyl)pyridin-3-yl)-1H- 1,2,3-triazole-4-carboxamide |
| 277 | 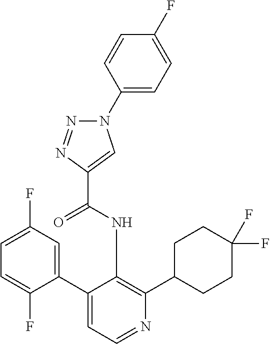 [SMILES: O=C(Nc1c(-c2cc(F)ccc2F)ccnc1C1CCC(F)(F)CC1)c1cn(-c2ccc(F)cc2)nn1] | N-(2-(4,4-difluorocyclohexyl)-4- (2,5-difluorophenyl)pyridin-3-yl)- 1-(4-fluorophenyl)-1H-1,2,3- triazole-4-carboxamide |
| 278 | 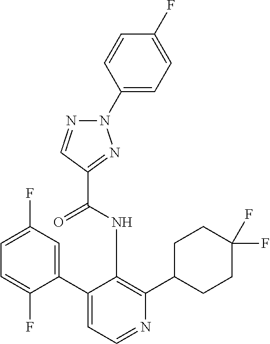 [SMILES: O=C(Nc1c(-c2cc(F)ccc2F)ccnc1C1CCC(F)(F)CC1)c1cnn(-c2ccc(F)cc2)n1] | N-(2-(4,4-difluorocyclohexyl)-4- (2,5-difluorophenyl)pyridin-3-yl)- 2-(4-fluorophenyl)-2H-1,2,3- triazole-4-carboxamide |
| 279 | 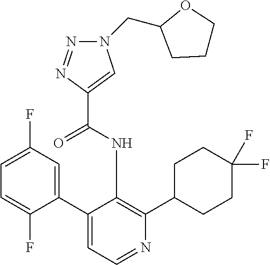 [SMILES: O=C(Nc1c(-c2cc(F)ccc2F)ccnc1C1CCC(F)(F)CC1)c1cn(CC2CCCO2)nn1] | N-(2-(4,4-difluorocyclohexyl)-4- (2,5-difluorophenyl)pyridin-3-yl)- 1-(rac-(tetrahydrofuran-2- yl)methyl)-1H-1,2,3-triazole-4- carboxamide |
| 280 | 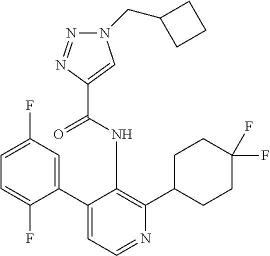 [SMILES: O=C(Nc1c(-c2cc(F)ccc2F)ccnc1C1CCC(F)(F)CC1)c1cn(CC2CCC2)nn1] | 1-(cyclobutylmethyl)-N-(2-(4,4- difluorocyclohexyl)-4-(2,5- difluorophenyl)pyridin-3-yl)-1H- 1,2,3-triazole-4-carboxamide |
| 281 | 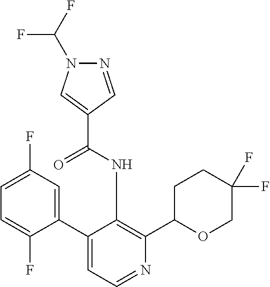 [SMILES: O=C(Nc1c(-c2cc(F)ccc2F)ccnc1C1CCC(F)(F)CO1)c1cnn(C(F)F)c1] | rac-1-(difluoromethyl)-N-(4-(2,5- difluorophenyl)-2-(5,5- difluorotetrahydro-2H-pyran-2- yl)pyridin-3-yl)-1H-pyrazole-4- carboxamide |
| 282 | 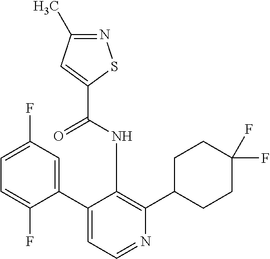 [SMILES: Cc1cc(C(=O)Nc2c(-c3cc(F)ccc3F)ccnc2C2CCC(F)(F)CC2)sn1] | N-(2-(4,4-difluorocyclohexyl)-4- (2,5-difluorophenyl)pyridin-3-yl)- 3-methylisothiazole-5- carboxamide |
| 283 | 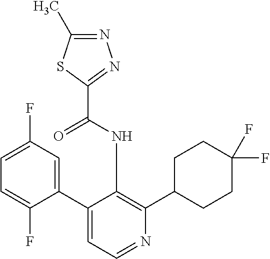 [SMILES: Cc1nnc(C(=O)Nc2c(-c3cc(F)ccc3F)ccnc2C2CCC(F)(F)CC2)s1] | N-(2-(4,4-difluorocyclohexyl)-4- (2,5-difluorophenyl)pyridin-3-yl)- 5-methyl-1,3,4-thiadiazole-2- carboxamide |
| 284 | 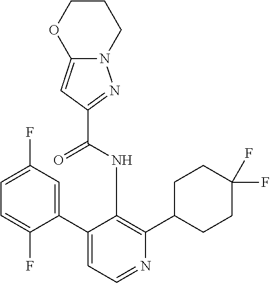 [SMILES: O=C(Nc1c(-c2cc(F)ccc2F)ccnc1C1CCC(F)(F)CC1)c1cc2n(n1)CCCO2] | N-(2-(4,4-difluorocyclohexyl)-4- (2,5-difluorophenyl)pyridin-3-yl)- 6,7-dihydro-5H-pyrazolo[5,1- b][1,3]oxazine-2-carboxamide |
| 285 | 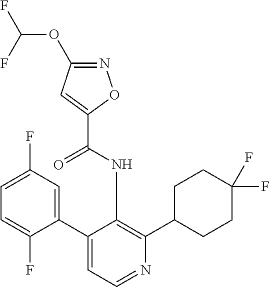 [SMILES: O=C(Nc1c(-c2cc(F)ccc2F)ccnc1C1CCC(F)(F)CC1)c1cc(OC(F)F)no1] | N-(2-(4,4-difluorocyclohexyl)-4- (2,5-difluorophenyl)pyridin-3-yl)- 3-(difluoromethoxy)isoxazole-5- carboxamide |
| 286 | 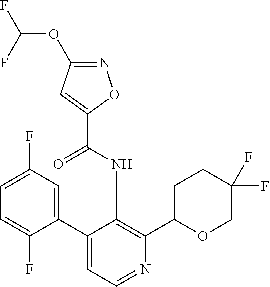 [SMILES: O=C(Nc1c(-c2cc(F)ccc2F)ccnc1C1CCC(F)(F)CO1)c1cc(OC(F)F)no1] | rac-3-(difluoromethoxy)-N-(4- (2,5-difluorophenyl)-2-(5,5- difluorotetrahydro-2H-pyran-2- yl)pyridin-3-yl)isoxazole-5- carboxamide |
| 287 | 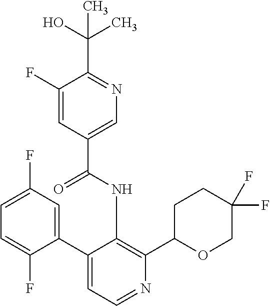 [SMILES: CC(C)(O)c1ncc(C(=O)Nc2c(-c3cc(F)ccc3F)ccnc2C2CCC(F)(F)CO2)cc1F] | rac-N-(4-(2,5-difluorophenyl)-2- (5,5-difluorotetrahydro-2H-pyran- 2-yl)pyridin-3-yl)-5-fluoro-6-(2- hydroxypropan-2-yl)nicotinamide |
| 288 | 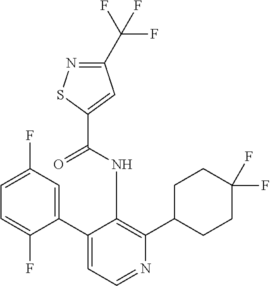 [SMILES: O=C(Nc1c(-c2cc(F)ccc2F)ccnc1C1CCC(F)(F)CC1)c1cc(C(F)(F)F)ns1] | N-(2-(4,4-difluorocyclohexyl)-4- (2,5-difluorophenyl)pyridin-3-yl)- 3-(trifluoromethyl)isothiazole-5- carboxamide |
| 289 | 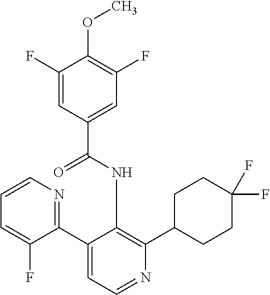 [SMILES: COc1c(F)cc(C(=O)Nc2c(-c3ncccc3F)ccnc2C2CCC(F)(F)CC2)cc1F] | N-(2′-(4,4-difluorocyclohexyl)-3- fluoro-[2,4′-bipyridin]-3′-yl)-3,5- difluoro-4-methoxybenzamide |
| 290 | 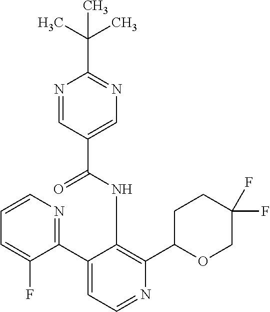 [SMILES: CC(C)(C)c1ncc(C(=O)Nc2c(-c3ncccc3F)ccnc2C2CCC(F)(F)CO2)cn1] | rac-2-(tert-butyl)-N-(2′-(5,5- difluorotetrahydro-2H-pyran-2- yl)-3-fluoro-[2,4′-bipyridin]-3′- yl)pyrimidine-5-carboxamide |
| 291 | 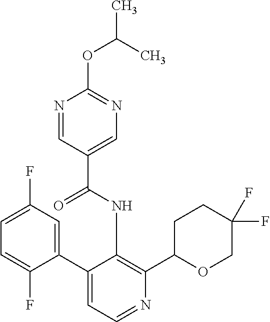 [SMILES: CC(C)Oc1ncc(C(=O)Nc2c(-c3cc(F)ccc3F)ccnc2C2CCC(F)(F)CO2)cn1] | rac-N-(4-(2,5-difluorophenyl)-2- (5,5-difluorotetrahydro-2H-pyran- 2-yl)pyridin-3-yl)-2- isopropoxypyrimidine-5- carboxamide |
| 292 | 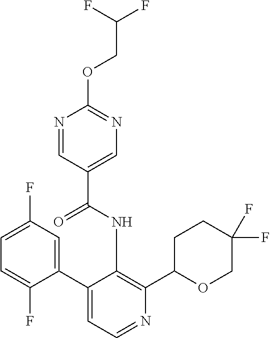 [SMILES: O=C(Nc1c(-c2cc(F)ccc2F)ccnc1C1CCC(F)(F)CO1)c1cnc(OCC(F)F)nc1] | rac-2-(2,2-difluoroethoxy)-N-(4- (2,5-difluorophenyl)-2-(5,5- difluorotetrahydro-2H-pyran-2- yl)pyridin-3-yl)pyrimidine-5- carboxamide |
| 293 | 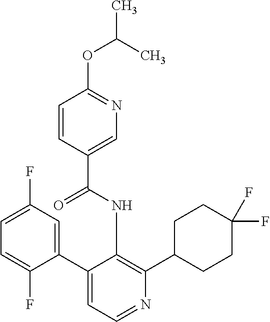 [SMILES: CC(C)Oc1ccc(C(=O)Nc2c(-c3cc(F)ccc3F)ccnc2C2CCC(F)(F)CC2)cn1] | N-(2-(4,4-difluorocyclohexyl)-4- (2,5-difluorophenyl)pyridin-3-yl)- 6-isopropoxynicotinamide |
| 294 | 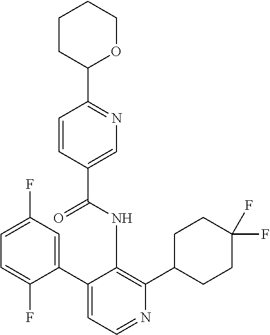 [SMILES: O=C(Nc1c(-c2cc(F)ccc2F)ccnc1C1CCC(F)(F)CC1)c1ccc(C2CCCCO2)nc1] | rac-N-(2-(4,4-difluorocyclohexyl)- 4-(2,5-difluorophenyl)pyridin-3- yl)-6-(tetrahydro-2H-pyran-2- yl)nicotinamide |
| 295 | 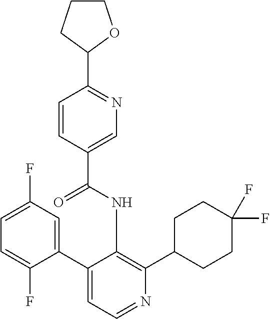 [SMILES: O=C(Nc1c(-c2cc(F)ccc2F)ccnc1C1CCC(F)(F)CC1)c1ccc(C2CCCO2)nc1] | rac-N-(2-(4,4-difluorocyclohexyl)- 4-(2,5-difluorophenyl)pyridin-3- yl)-6-(tetrahydrofuran-2- yl)nicotinamide |
| 296 | 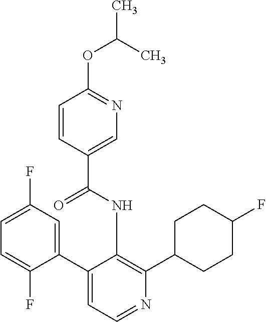 [SMILES: CC(C)Oc1ccc(C(=O)Nc2c(-c3cc(F)ccc3F)ccnc2C2CCC(F)CC2)cn1] | N-(4-(2,5-difluorophenyl)-2-(4- fluorocyclohexyl)pyridin-3-yl)-6- isopropoxynicotinamide P1 |
| 297 | 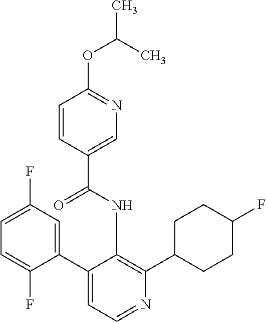 [SMILES: CC(C)Oc1ccc(C(=O)Nc2c(-c3cc(F)ccc3F)ccnc2C2CCC(F)CC2)cn1] | N-(4-(2,5-difluorophenyl)-2-(4- fluorocyclohexyl)pyridin-3-yl)-6- isopropoxynicotinamide P2 |
| 298 | 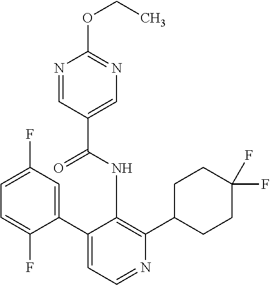 [SMILES: CCOc1ncc(C(=O)Nc2c(-c3cc(F)ccc3F)ccnc2C2CCC(F)(F)CC2)cn1] | N-(2-(4,4-difluorocyclohexyl)-4- (2,5-difluorophenyl)pyridin-3-yl)- 2-ethoxypyrimidine-5- carboxamide |
| 299 | 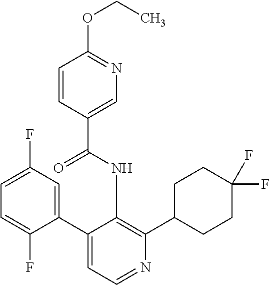 [SMILES: CCOc1ccc(C(=O)Nc2c(-c3cc(F)ccc3F)ccnc2C2CCC(F)(F)CC2)cn1] | N-(2-(4,4-difluorocyclohexyl)-4- (2,5-difluorophenyl)pyridin-3-yl)- 6-ethoxynicotinamide |
| 300 | 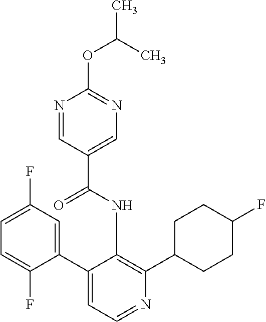 [SMILES: CC(C)Oc1ncc(C(=O)Nc2c(-c3cc(F)ccc3F)ccnc2C2CCC(F)CC2)cn1] | N-(4-(2,5-difluorophenyl)-2-(4- fluorocyclohexyl)pyridin-3-yl)-2- isopropoxypyrimidine-5- carboxamide P1 |
| 301 | 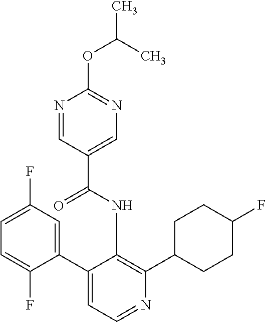 [SMILES: CC(C)Oc1ncc(C(=O)Nc2c(-c3cc(F)ccc3F)ccnc2C2CCC(F)CC2)cn1] | N-(4-(2,5-difluorophenyl)-2-(4- fluorocyclohexyl)pyridin-3-yl)-2- isopropoxypyrimidine-5- carboxamide P2 |
| 302 | 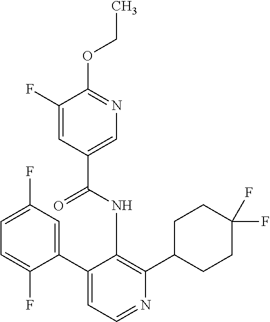 [SMILES: CCOc1ncc(C(=O)Nc2c(-c3cc(F)ccc3F)ccnc2C2CCC(F)(F)CC2)cc1F] | N-(2-(4,4-difluorocyclohexyl)-4- (2,5-difluorophenyl)pyridin-3-yl)- 6-ethoxy-5-fluoronicotinamide |
| 303 | 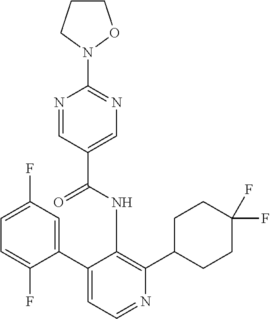 [SMILES: O=C(Nc1c(-c2cc(F)ccc2F)ccnc1C1CCC(F)(F)CC1)c1cnc(N2CCCO2)nc1] | N-(2-(4,4-difluorocyclohexyl)-4- (2,5-difluorophenyl)pyridin-3-yl)- 2-(isoxazolidin-2-yl)pyrimidine-5- carboxamide |
| 304 | 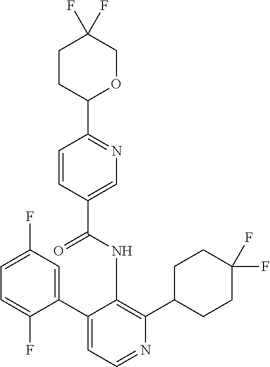 [SMILES: O=C(Nc1c(-c2cc(F)ccc2F)ccnc1C1CCC(F)(F)CC1)c1ccc(C2CCC(F)(F)CO2)nc1] | rac-N-(2-(4,4-difluorocyclohexyl)- 4-(2,5-difluorophenyl)pyridin-3- yl)-6-(5,5-difluorotetrahydro-2H- pyran-2-yl)nicotinamide |
| 305 | 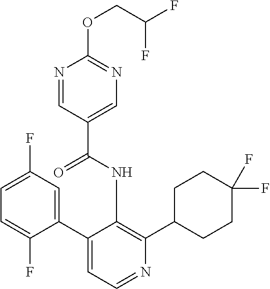 [SMILES: O=C(Nc1c(-c2cc(F)ccc2F)ccnc1C1CCC(F)(F)CC1)c1cnc(OCC(F)F)nc1] | N-(2-(4,4-difluorocyclohexyl)-4- (2,5-difluorophenyl)pyridin-3-yl)- 2-(2,2-difluoroethoxy)pyrimidine- 5-carboxamide |
| 306 | 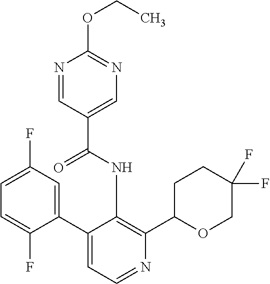 [SMILES: CCOc1ncc(C(=O)Nc2c(-c3cc(F)ccc3F)ccnc2C2CCC(F)(F)CO2)cn1] | rac-N-(4-(2,5-difluorophenyl)-2- (5,5-difluorotetrahydro-2H-pyran- 2-yl)pyridin-3-yl)-2- ethoxypyrimidine-5-carboxamide |
| 307 | 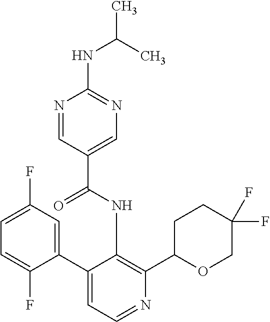 [SMILES: CC(C)Nc1ncc(C(=O)Nc2c(-c3cc(F)ccc3F)ccnc2C2CCC(F)(F)CO2)cn1] | rac-N-(4-(2,5-difluorophenyl)-2- (5,5-difluorotetrahydro-2H-pyran- 2-yl)pyridin-3-yl)-2- (isopropylamino)pyrimidine-5- carboxamide |
| 308 | 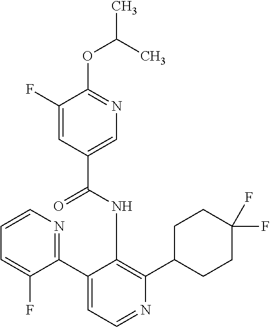 [SMILES: CC(C)Oc1ncc(C(=O)Nc2c(-c3ncccc3F)ccnc2C2CCC(F)(F)CC2)cc1F] | N-(2′-(4,4-difluorocyclohexyl)-3- fluoro-[2,4′-bipyridin]-3′-yl)-5- fluoro-6-isopropoxynicotinamide |
| 309 | 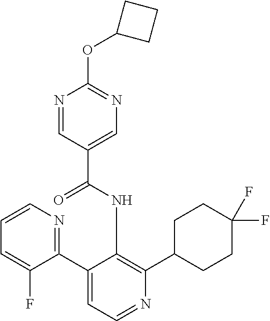 [SMILES: O=C(Nc1c(-c2ncccc2F)ccnc1C1CCC(F)(F)CC1)c1cnc(OC2CCC2)nc1] | 2-cyclobutoxy-N-(2′-(4,4- difluorocyclohexyl)-3-fluoro-[2,4′- bipyridin]-3′-yl)pyrimidine-5- carboxamide |
| 310 | 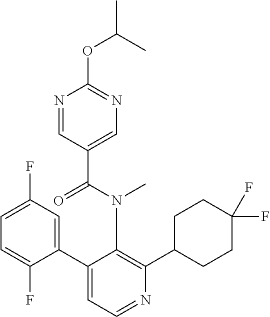 [SMILES: CC(C)Oc1ncc(C(=O)N(C)c2c(-c3cc(F)ccc3F)ccnc2C2CCC(F)(F)CC2)cn1] | N-(2-(4,4-difluorocyclohexyl)-4- (2,5-difluorophenyl)pyridin-3-yl)- 2-isopropoxy-N- methylpyrimidine-5-carboxamide |
| 311 | 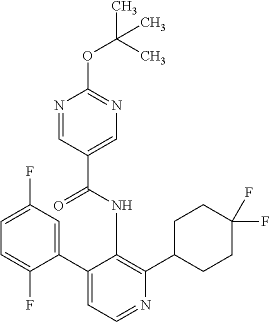 [SMILES: CC(C)(C)Oc1ncc(C(=O)Nc2c(-c3cc(F)ccc3F)ccnc2C2CCC(F)(F)CC2)cn1] | 2-(tert-butoxy)-N-(2-(4,4- difluorocyclohexyl)-4-(2,5- difluorophenyl)pyridin-3- yl)pyrimidine-5-carboxamide |
| 312 | 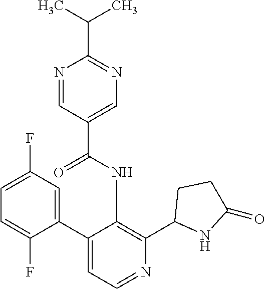 [SMILES: CC(C)c1ncc(C(=O)Nc2c(-c3cc(F)ccc3F)ccnc2C2CCC(=O)N2)cn1] | rac-N-(4-(2,5-difluorophenyl)-2- (5-oxopyrrolidin-2-yl)pyridin-3- yl)-2-isopropylpyrimidine-5- carboxamide |
| 313 | 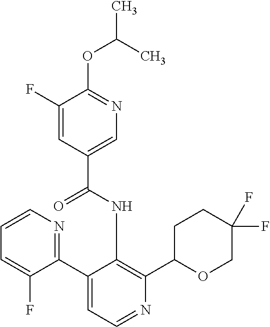 [SMILES: CC(C)Oc1ncc(C(=O)Nc2c(-c3ncccc3F)ccnc2C2CCC(F)(F)CO2)cc1F] | rac-N-(2′-(5,5-difluorotetrahydro- 2H-pyran-2-yl)-3-fluoro-[2,4′- bipyridin]-3′-yl)-5-fluoro-6- isopropoxynicotinamide |
| 314 | 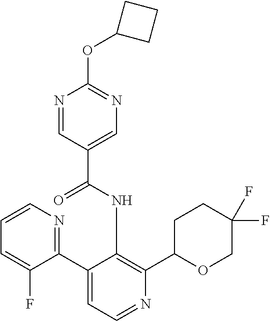 [SMILES: O=C(Nc1c(-c2ncccc2F)ccnc1C1CCC(F)(F)CO1)c1cnc(OC2CCC2)nc1] | rac-2-cyclobutoxy-N-(2′-(5,5- difluorotetrahydro-2H-pyran-2- yl)-3-fluoro-[2,4′-bipyridin]-3′- yl)pyrimidine-5-carboxamide |
| 315 | 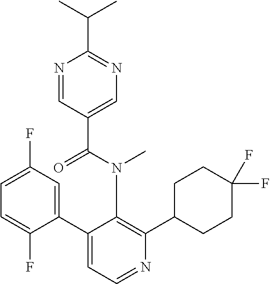 [SMILES: CC(C)c1ncc(C(=O)N(C)c2c(-c3cc(F)ccc3F)ccnc2C2CCC(F)(F)CC2)cn1] | N-(2-(4,4-difluorocyclohexyl)-4- (2,5-difluorophenyl)pyridin-3-yl)- 2-isopropyl-N-methylpyrimidine- 5-carboxamide |
| 316 | 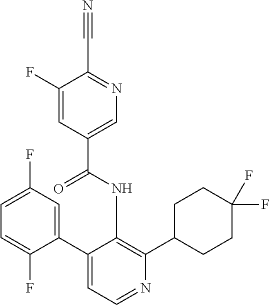 [SMILES: N#Cc1ncc(C(=O)Nc2c(-c3cc(F)ccc3F)ccnc2C2CCC(F)(F)CC2)cc1F] | 6-cyano-N-(2-(4,4- difluorocyclohexyl)-4-(2,5- difluorophenyl)pyridin-3-yl)-5- fluoronicotinamide |
| 317 | 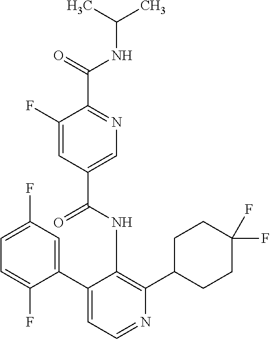 [SMILES: CC(C)NC(=O)c1ncc(C(=O)Nc2c(-c3cc(F)ccc3F)ccnc2C2CCC(F)(F)CC2)cc1F] | N-5-(2-(4,4-difluorocyclohexyl)-4- (2,5-difluorophenyl)pyridin-3-yl)- 3-fluoro-N²-isopropylpyridine- 2,5-dicarboxamide |
| 318 | 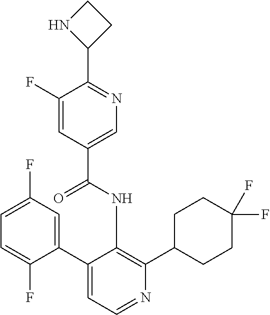 [SMILES: O=C(Nc1c(-c2cc(F)ccc2F)ccnc1C1CCC(F)(F)CC1)c1cnc(C2CCN2)c(F)c1] | rac-6-(azetidin-2-yl)-N-(2-(4,4- difluorocyclohexyl)-4-(2,5- difluorophenyl)pyridin-3-yl)-5- fluoronicotinamide |
| 319 | 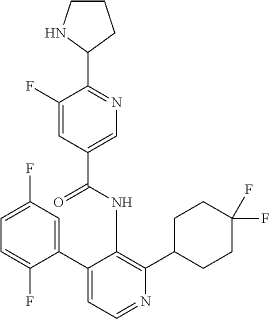 [SMILES: O=C(Nc1c(-c2cc(F)ccc2F)ccnc1C1CCC(F)(F)CC1)c1cnc(C2CCCN2)c(F)c1] | rac-N-(2-(4,4-difluorocyclohexyl)- 4-(2,5-difluorophenyl)pyridin-3- yl)-5-fluoro-6-(pyrrolidin-2- yl)nicotinamide trifluoroacetic acid salt |
| 320 | 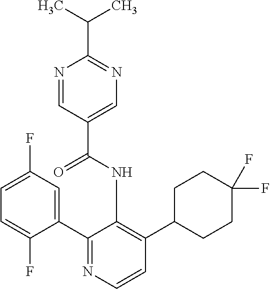 [SMILES: CC(C)c1ncc(C(=O)Nc2c(C3CCC(F)(F)CC3)ccnc2-c2cc(F)ccc2F)cn1] | N-(4-(4,4-difluorocyclohexyl)-2- (2,5-difluorophenyl)pyridin-3-yl)- 2-isopropylpyrimidine-5- carboxamide |
| 321 | 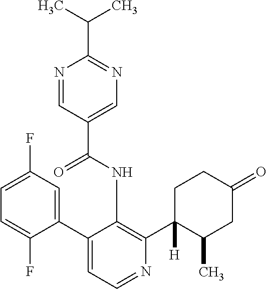 [SMILES: CC(C)c1ncc(C(=O)Nc2c(-c3cc(F)ccc3F)ccnc2[C@@H]2CCC(=O)C[C@H]2C)cn1] | rac-N-(4-(2,5-difluorophenyl)-2- ((anti)-2-methyl-4- oxocyclohexyl)pyridin-3-yl)-2- isopropylpyrimidine-5- carboxamide |
| 322 | 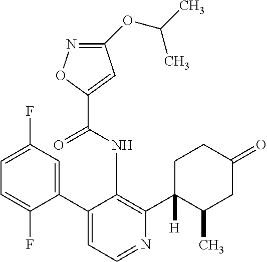 [SMILES: CC(C)Oc1cc(C(=O)Nc2c(-c3cc(F)ccc3F)ccnc2[C@@H]2CCC(=O)C[C@H]2C)on1] | rac-N-(4-(2,5-difluorophenyl)-2- ((anti)-2-methyl-4- oxocyclohexyl)pyridin-3-yl)-2- isopropylpyrimidine-5- carboxamide |
| 323 | 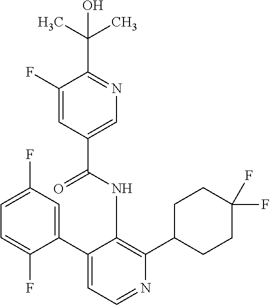 [SMILES: CC(C)(O)c1ncc(C(=O)Nc2c(-c3cc(F)ccc3F)ccnc2C2CCC(F)(F)CC2)cc1F] | N-(2-(4,4-difluorocyclohexyl)-4- (2,5-difluorophenyl)pyridin-3-yl)- 5-fluoro-6-(2-hydroxypropan-2- yl)nicotinamide |
| 324 | 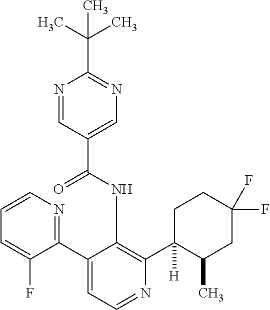 [SMILES: C[C@@H]1CC(F)(F)CC[C@@H]1c1nccc(-c2ncccc2F)c1NC(=O)c1cnc(C(C)(C)C)nc1] | 2-(tert-butyl)-N-(2′-(rac-(syn)-4,4- difluoro-2-methylcyclohexyl)-3- fluoro-[2,4′-bipyridin]-3′- yl)pyrimidine-5-carboxamide |
| 325 | 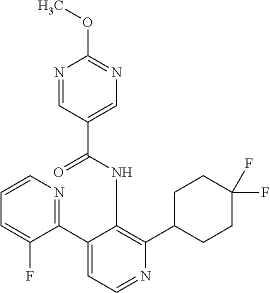 [SMILES: COc1ncc(C(=O)Nc2c(-c3ncccc3F)ccnc2C2CCC(F)(F)CC2)cn1] | N-(2′-(4,4-difluorocyclohexyl)-3- fluoro-[2,4′-bipyridin]-3′-yl)-2- methoxypyrimidine-5- carboxamide |
| 326 | 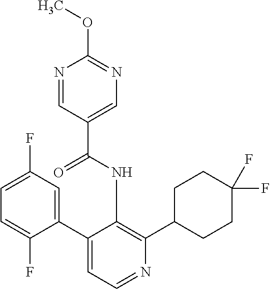 [SMILES: COc1ncc(C(=O)Nc2c(-c3cc(F)ccc3F)ccnc2C2CCC(F)(F)CC2)cn1] | N-(2-(4,4-difluorocyclohexyl)-4- (2,5-difluorophenyl)pyridin-3-yl)- 2-methoxypyrimidine-5- carboxamide |
| 327 | 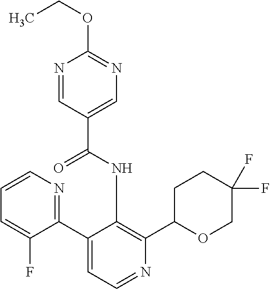 [SMILES: CCOc1ncc(C(=O)Nc2c(-c3ncccc3F)ccnc2C2CCC(F)(F)CO2)cn1] | rac-N-(2′-(5,5-difluorotetrahydro- 2H-pyran-2-yl)-3-fluoro-[2,4′- bipyridin]-3′-yl)-2- ethoxypyrimidine-5-carboxamide |
| 328 | 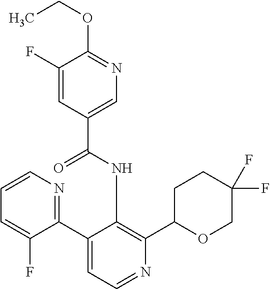 [SMILES: CCOc1ncc(C(=O)Nc2c(-c3ncccc3F)ccnc2C2CCC(F)(F)CO2)cc1F] | rac-N-(2′-(5,5-difluorotetrahydro- 2H-pyran-2-yl)-3-fluoro-[2,4′- bipyridin]-3′-yl)-6-ethoxy-5- fluoronicotinamide |
| 329 | 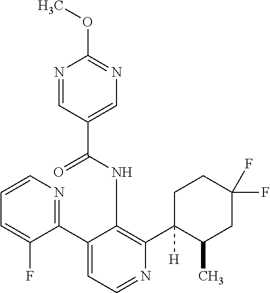 [SMILES: COc1ncc(C(=O)Nc2c(-c3ncccc3F)ccnc2[C@H]2CCC(F)(F)C[C@H]2C)cn1] | N-(2′-(rac-(syn)-4,4-difluoro-2- methylcyclohexyl)-3-fluoro-[2,4′- bipyridin]-3′-yl)-2- methoxypyrimidine-5- carboxamide |
| 330 | 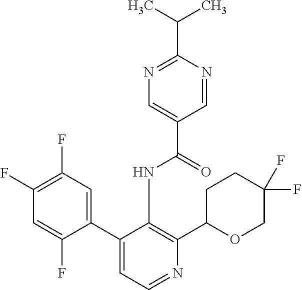 [SMILES: CC(C)c1ncc(C(=O)Nc2c(-c3cc(F)c(F)cc3F)ccnc2C2CCC(F)(F)CO2)cn1] | rac-N-(2-(5,5-difluorotetrahydro- 2H-pyran-2-yl)-4-(2,4,5- trifluorophenyl)pyridin-3-yl)-2- isopropylpyrimidine-5- carboxamide |
| 331 | 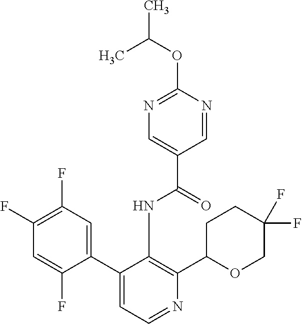 [SMILES: CC(C)Oc1ncc(C(=O)Nc2c(-c3cc(F)c(F)cc3F)ccnc2C2CCC(F)(F)CO2)cn1] | rac-N-(2-(5,5-difluorotetrahydro- 2H-pyran-2-yl)-4-(2,4,5- trifluorophenyl)pyridin-3-yl)-2- isopropoxypyrimidine-5- carboxamide |
| 332 | 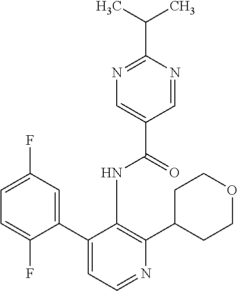 [SMILES: CC(C)c1ncc(C(=O)Nc2c(-c3cc(F)ccc3F)ccnc2C2CCOCC2)cn1] | N-(4-(2,5-difluorophenyl)-2- (tetrahydro-2H-pyran-4- yl)pyridin-3-yl)-2- isopropylpyrimidine-5- carboxamide |
| 333 | 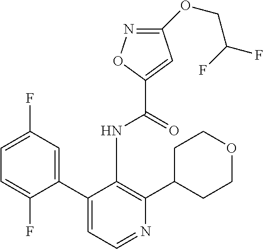 [SMILES: O=C(Nc1c(-c2cc(F)ccc2F)ccnc1C1CCOCC1)c1cc(OCC(F)F)no1] | 3-(2,2-difluoroethoxy)-N-(4-(2,5- difluorophenyl)-2-(tetrahydro-2H- pyran-4-yl)pyridin-3-yl)isoxazole- 5-carboxamide |
| 334 | 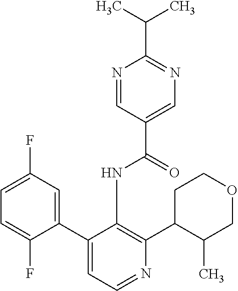 [SMILES: CC(C)c1ncc(C(=O)Nc2c(-c3cc(F)ccc3F)ccnc2C2CCOCC2C)cn1] | N-(4-(2,5-difluorophenyl)-2- ((3S,4R)-3-methyltetrahydro-2H- pyran-4-yl)pyridin-3-yl)-2- isopropylpyrimidine-5- carboxamide (racemic mix) |
| 335 | 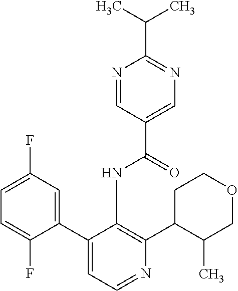 [SMILES: CC(C)c1ncc(C(=O)Nc2c(-c3cc(F)ccc3F)ccnc2C2CCOCC2C)cn1] | N-(4-(2,5-difluorophenyl)-2- ((3S,4R)-3-methyltetrahydro-2H- pyran-4-yl)pyridin-3-yl)-2- isopropylpyrimidine-5- carboxamide P1 |
| 336 | 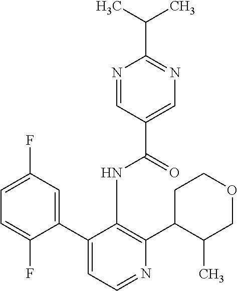 [SMILES: CC(C)c1ncc(C(=O)Nc2c(-c3cc(F)ccc3F)ccnc2C2CCOCC2C)cn1] | N-(4-(2,5-difluorophenyl)-2- ((3S,4R)-3-methyltetrahydro-2H- pyran-4-yl)pyridin-3-yl)-2- isopropylpyrimidine-5- carboxamide P2 |
| 337 | 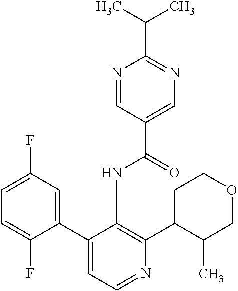 [SMILES: CC(C)c1ncc(C(=O)Nc2c(-c3cc(F)ccc3F)ccnc2C2CCOCC2C)cn1] | N-(4-(2,5-difluorophenyl)-2- ((3S,4R)-3-methyltetrahydro-2H- pyran-4-yl)pyridin-3-yl)-2- isopropylpyrimidine-5- carboxamide P3 |
| 338 | 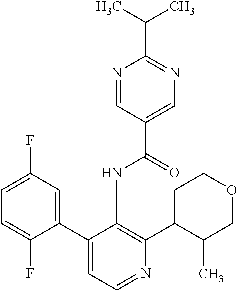 [SMILES: CC(C)c1ncc(C(=O)Nc2c(-c3cc(F)ccc3F)ccnc2C2CCOCC2C)cn1] | N-(4-(2,5-difluorophenyl)-2- ((3S,4R)-3-methyltetrahydro-2H- pyran-4-yl)pyridin-3-yl)-2- isopropylpyrimidine-5- carboxamide P4 |
| 339 | 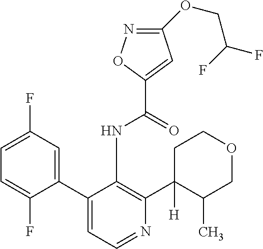 [SMILES: CC1COCCC1c1nccc(-c2cc(F)ccc2F)c1NC(=O)c1cc(OCC(F)F)no1] | 3-(2,2-difluoroethoxy)-N-(4-(2,5- difluorophenyl)-3- methyltetrahydro-2H-pyran-4- yl)pyridin-3-yl)isoxazole-5- carboxamide P1 |
| 340 | 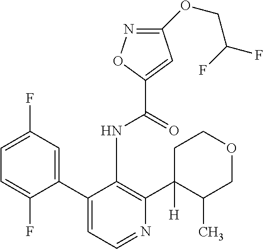 [SMILES: CC1COCCC1c1nccc(-c2cc(F)ccc2F)c1NC(=O)c1cc(OCC(F)F)no1] | 3-(2,2-difluoroethoxy)-N-(4-(2,5- difluorophenyl)-3- methyltetrahydro-2H-pyran-4- yl)pyridin-3-yl)isoxazole-5- carboxamide P2 |
| 341 | 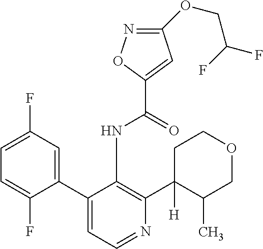 [SMILES: CC1COCCC1c1nccc(-c2cc(F)ccc2F)c1NC(=O)c1cc(OCC(F)F)no1] | 3-(2,2-difluoroethoxy)-N-(4-(2,5- difluorophenyl)-3- methyltetrahydro-2H-pyran-4- yl)pyridin-3-yl)isoxazole-5- carboxamide P3 |
| 342 | 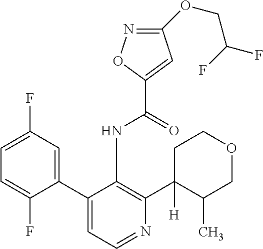 [SMILES: CC1COCCC1c1nccc(-c2cc(F)ccc2F)c1NC(=O)c1cc(OCC(F)F)no1] | 3-(2,2-difluoroethoxy)-N-(4-(2,5- difluorophenyl)-3- methyltetrahydro-2H-pyran-4- yl)pyridin-3-yl)isoxazole-5- carboxamide P4 |
| 343 | 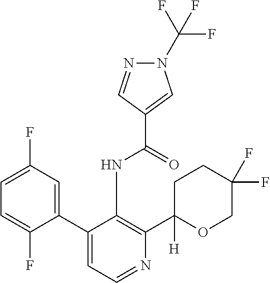 [SMILES: O=C(Nc1c(-c2cc(F)ccc2F)ccnc1C1CCC(F)(F)CO1)c1cnn(C(F)(F)F)c1] | N-(4-(2,5-difluorophenyl)-2-(5,5- difluorotetrahydro-2H-pyran-2- yl)pyridin-3-yl)-1- (trifluoromethyl)-1H-pyrazole-4- carboxamide P1 |
| 344 | 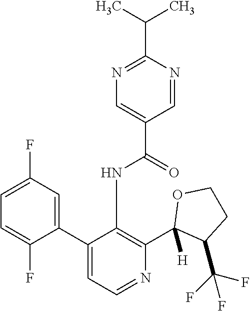 [SMILES: CC(C)c1ncc(C(=O)Nc2c(-c3cc(F)ccc3F)ccnc2[C@@H]2OCC[C@H]2C(F)(F)F)cn1] | N-(4-(2,5-difluorophenyl)3- (trifluoromethyl)tetrahydrofuran- 2-yl)pyridin-3-yl)-2- isopropylpyrimidine-5- carboxamide P1 |
| 345 | 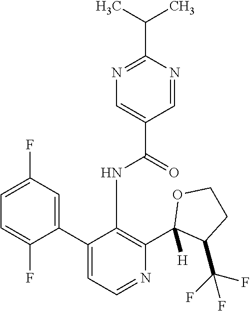 [SMILES: CC(C)c1ncc(C(=O)Nc2c(-c3cc(F)ccc3F)ccnc2[C@@H]2OCC[C@H]2C(F)(F)F)cn1] | N-(4-(2,5-difluorophenyl)3- (trifluoromethyl)tetrahydrofuran- 2-yl)pyridin-3-yl)-2- isopropylpyrimidine-5- carboxamide P2 |
| 346 | 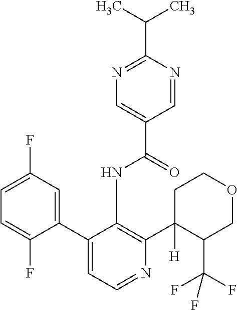 [SMILES: CC(C)c1ncc(C(=O)Nc2c(-c3cc(F)ccc3F)ccnc2C2CCOCC2C(F)(F)F)cn1] | N-(4-(2,5-difluorophenyl)-2-(3- (trifluoromethyl)tetrahydro-2H- pyran-4-yl)pyridin-3-yl)-2- isopropylpyrimidine-5- carboxamide P1 |
| 347 | 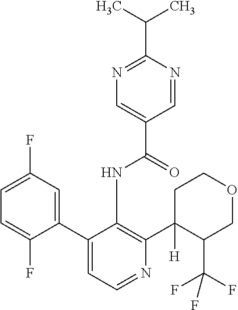 [SMILES: CC(C)c1ncc(C(=O)Nc2c(-c3cc(F)ccc3F)ccnc2C2CCOCC2C(F)(F)F)cn1] | N-(4-(2,5-difluorophenyl)-2-(3- (trifluoromethyl)tetrahydro-2H- pyran-4-yl)pyridin-3-yl)-2- isopropylpyrimidine-5- carboxamide P2 |
| 348 | 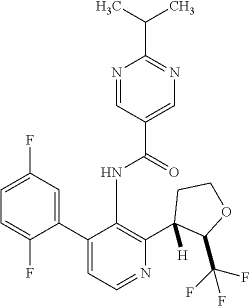 [SMILES: CC(C)c1ncc(C(=O)Nc2c(-c3cc(F)ccc3F)ccnc2[C@@H]2CCO[C@H]2C(F)(F)F)cn1] | N-(4-(2,5-difluorophenyl)-2-(rac- anti-2- (trifluoromethyl)tetrahydrofuran- 3-yl)pyridin-3-yl)-2- isopropylpyrimidine-5- carboxamide |
| 349 | 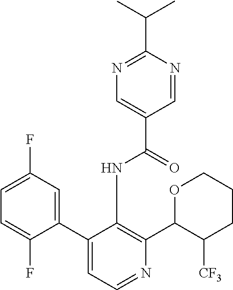 [SMILES: CC(C)c1ncc(C(=O)Nc2c(-c3cc(F)ccc3F)ccnc2C2OCCCC2C(F)(F)F)cn1] | N-(4-(2,5-difluorophenyl)-2-(3- (trifluoromethyl)tetrahydro-2H- pyran-2-yl)pyridin-3-yl)-2- isopropylpyrimidine-5- carboxamide P1 |
| 350 | 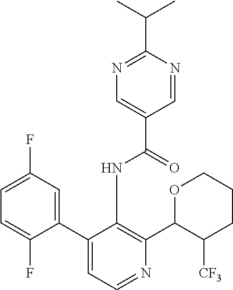 [SMILES: CC(C)c1ncc(C(=O)Nc2c(-c3cc(F)ccc3F)ccnc2C2OCCCC2C(F)(F)F)cn1] | N-(4-(2,5-difluorophenyl)-2-(3- (trifluoromethyl)tetrahydro-2H- pyran-2-yl)pyridin-3-yl)-2- isopropylpyrimidine-5- carboxamide P2 |
| 351 | 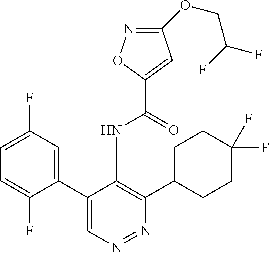 [SMILES: O=C(Nc1c(-c2cc(F)ccc2F)cnnc1C1CCC(F)(F)CC1)c1cc(OCC(F)F)no1] | N-(3-(4,4-difluorocyclohexyl)-5- (2,5-difluorophenyl)pyridazin-4- yl)-3-(2,2- difluoroethoxy)isoxazole-5- carboxamide |
| 352 | 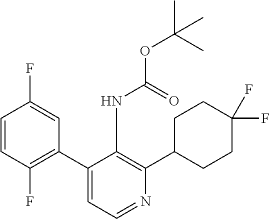 [SMILES: CC(C)(C)OC(=O)Nc1c(-c2cc(F)ccc2F)ccnc1C1CCC(F)(F)CC1] | N-[2-(4,4-difluorocyclohexyl)-4- (2,5-difluorophenyl)-3- pyridyl]carbamate |
| 353 | 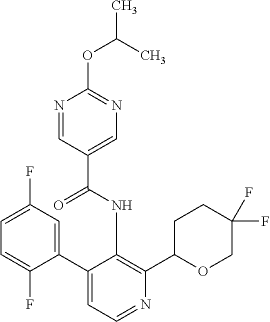 [SMILES: CC(C)Oc1ncc(C(=O)Nc2c(-c3cc(F)ccc3F)ccnc2C2CCC(F)(F)CO2)cn1] | N-(4-(2,5-difluorophenyl)-2-(5,5- difluorotetrahydro-2H-pyran-2- yl)pyridin-3-yl)-2- isopropoxypyrimidine-5- carboxamide P1 |
| 354 | 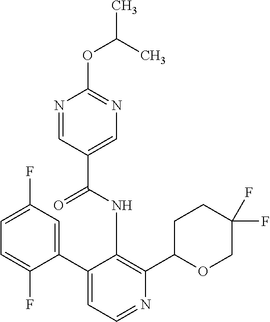 [SMILES: CC(C)Oc1ncc(C(=O)Nc2c(-c3cc(F)ccc3F)ccnc2C2CCC(F)(F)CO2)cn1] | N-(4-(2,5-difluorophenyl)-2-(5,5- difluorotetrahydro-2H-pyran-2- yl)pyridin-3-yl)-2- isopropoxypyrimidine-5- carboxamide P2 |
| 355 | 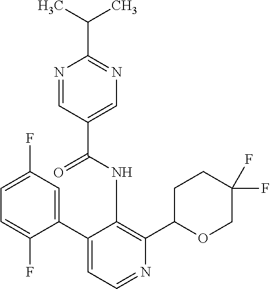 [SMILES: CC(C)c1ncc(C(=O)Nc2c(-c3cc(F)ccc3F)ccnc2C2CCC(F)(F)CO2)cn1] | N-(4-(2,5-difluorophenyl)-2-(5,5- difluorotetrahydro-2H-pyran-2- yl)pyridin-3-yl)-2- isopropylpyrimidine-5- carboxamide P1 |
| 356 | 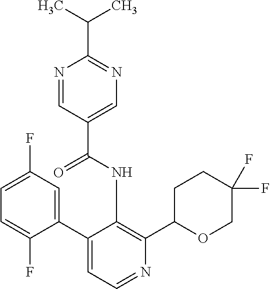 [SMILES: CC(C)c1ncc(C(=O)Nc2c(-c3cc(F)ccc3F)ccnc2C2CCC(F)(F)CO2)cn1] | N-(4-(2,5-difluorophenyl)-2-(5,5- difluorotetrahydro-2H-pyran-2- yl)pyridin-3-yl)-2- isopropylpyrimidine-5- carboxamide P2 |
| 357 | 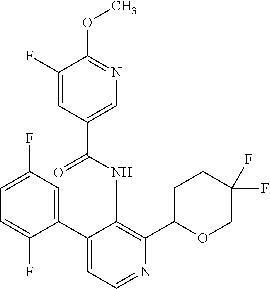 [SMILES: COc1ncc(C(=O)Nc2c(-c3cc(F)ccc3F)ccnc2C2CCC(F)(F)CO2)cc1F] | N-(4-(2,5-difluorophenyl)-2-(5,5- difluorotetrahydro-2H-pyran-2- yl)pyridin-3-yl)-5-fluoro-6- methoxynicotinamide P1 |
| 358 | 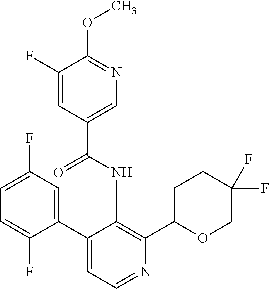 [SMILES: COc1ncc(C(=O)Nc2c(-c3cc(F)ccc3F)ccnc2C2CCC(F)(F)CO2)cc1F] | N-(4-(2,5-difluorophenyl)-2-(5,5- difluorotetrahydro-2H-pyran-2- yl)pyridin-3-yl)-5-fluoro-6- methoxynicotinamide P2 |
| 359 | 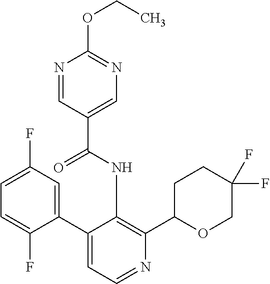 [SMILES: CCOc1ncc(C(=O)Nc2c(-c3cc(F)ccc3F)ccnc2C2CCC(F)(F)CO2)cn1] | N-(4-(2,5-difluorophenyl)-2-(5,5- difluorotetrahydro-2H-pyran-2- yl)pyridin-3-yl)-2- ethoxypyrimidine-5-carboxamide P1 |
| 360 | 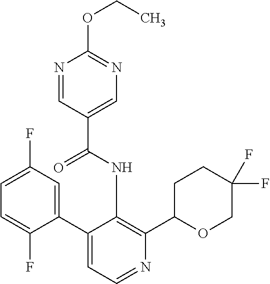 [SMILES: CCOc1ncc(C(=O)Nc2c(-c3cc(F)ccc3F)ccnc2C2CCC(F)(F)CO2)cn1] | N-(4-(2,5-difluorophenyl)-2-(5,5- difluorotetrahydro-2H-pyran-2- yl)pyridin-3-yl)-2- ethoxypyrimidine-5-carboxamide P2 |
| 361 | 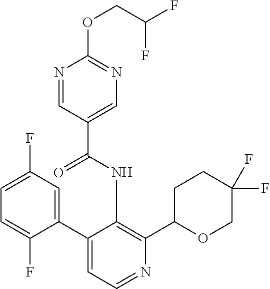 [SMILES: O=C(Nc1c(-c2cc(F)ccc2F)ccnc1C1CCC(F)(F)CO1)c1cnc(OCC(F)F)nc1] | 2-(2,2-difluoroethoxy)-N-(4-(2,5- difluorophenyl)-2-(5,5- difluorotetrahydro-2H-pyran-2- yl)pyridin-3-yl)pyrimidine-5- carboxamide P1 |
| 362 | 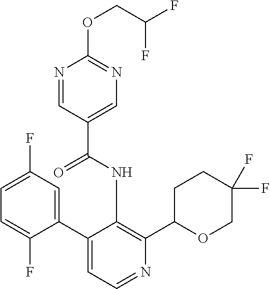 [SMILES: O=C(Nc1c(-c2cc(F)ccc2F)ccnc1C1CCC(F)(F)CO1)c1cnc(OCC(F)F)nc1] | 2-(2,2-difluoroethoxy)-N-(4-(2,5- difluorophenyl)-2-(5,5- difluorotetrahydro-2H-pyran-2- yl)pyridin-3-yl)pyrimidine-5- carboxamide P2 |
| 363 | 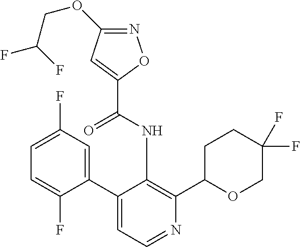 [SMILES: O=C(Nc1c(-c2cc(F)ccc2F)ccnc1C1CCC(F)(F)CO1)c1cc(OCC(F)F)no1] | 3-(2,2-difluoroethoxy)-N-(4-(2,5- difluorophenyl)-2-(5,5- difluorotetrahydro-2H-pyran-2- yl)pyridin-3-yl)isoxazole-5- carboxamide P1 |
| 364 | 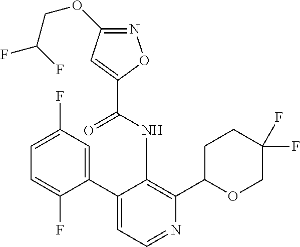 [SMILES: O=C(Nc1c(-c2cc(F)ccc2F)ccnc1C1CCC(F)(F)CO1)c1cc(OCC(F)F)no1] | 3-(2,2-difluoroethoxy)-N-(4-(2,5- difluorophenyl)-2-(5,5- difluorotetrahydro-2H-pyran-2- yl)pyridin-3-yl)isoxazole-5- carboxamide P2 |
| 365 | 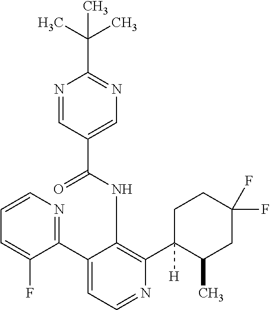 [SMILES: C[C@@H]1CC(F)(F)CC[C@@H]1c1nccc(-c2ncccc2F)c1NC(=O)c1cnc(C(C)(C)C)nc1] | 2-(tert-butyl)-N-(2′-(syn)-4,4- difluoro-2-methylcyclohexyl)-3- fluoro-[2,4′-bipyridin]-3′- yl)pyrimidine-5-carboxamide P1 |
| 366 | 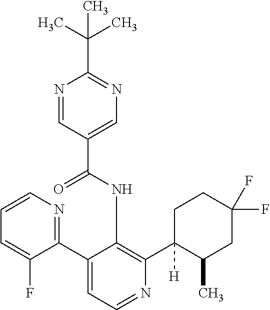 [SMILES: C[C@@H]1CC(F)(F)CC[C@@H]1c1nccc(-c2ncccc2F)c1NC(=O)c1cnc(C(C)(C)C)nc1] | 2-(tert-butyl)-N-(2′-(syn)-4,4- difluoro-2-methylcyclohexyl)-3- fluoro-[2,4′-bipyridin]-3′- yl)pyrimidine-5-carboxamide P2 |
| 367 | 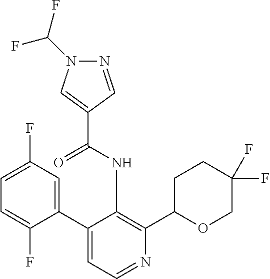 [SMILES: O=C(Nc1c(-c2cc(F)ccc2F)ccnc1C1CCC(F)(F)CO1)c1cnn(C(F)F)c1] | 1-(difluoromethyl)-N-(4-(2,5- difluorophenyl)-2-(5,5- difluorotetrahydro-2H-pyran-2- yl)pyridin-3-yl)-1H-pyrazole-4- carboxamide P1 |
| 368 | 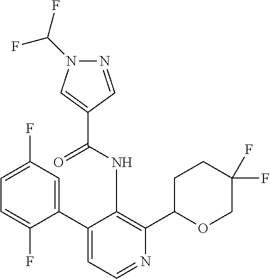 [SMILES: O=C(Nc1c(-c2cc(F)ccc2F)ccnc1C1CCC(F)(F)CO1)c1cnn(C(F)F)c1] | 1-(difluoromethyl)-N-(4-(2,5- difluorophenyl)-2-(5,5- difluorotetrahydro-2H-pyran-2- yl)pyridin-3-yl)-1H-pyrazole-4- carboxamide P2 |
| 369 | 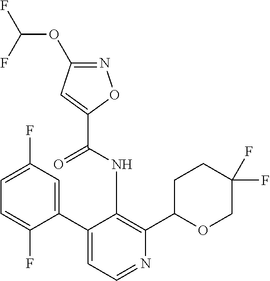 [SMILES: O=C(Nc1c(-c2cc(F)ccc2F)ccnc1C1CCC(F)(F)CO1)c1cc(OC(F)F)no1] | 3-(difluoromethoxy)-N-(4-(2,5- difluorophenyl)-2-(5,5- difluorotetrahydro-2H-pyran-2- yl)pyridin-3-yl)isoxazole-5- carboxamide P1 |
| 370 | 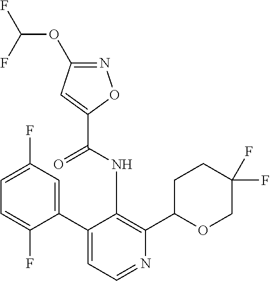 [SMILES: O=C(Nc1c(-c2cc(F)ccc2F)ccnc1C1CCC(F)(F)CO1)c1cc(OC(F)F)no1] | 3-(difluoromethoxy)-N-(4-(2,5- difluorophenyl)-2-(5,5- difluorotetrahydro-2H-pyran-2- yl)pyridin-3-yl)isoxazole-5- carboxamide P2 |
| 371 | 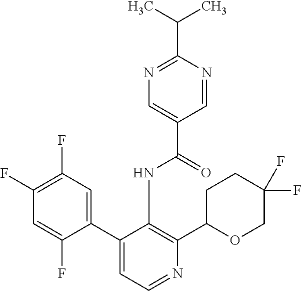 [SMILES: CC(C)c1ncc(C(=O)Nc2c(-c3cc(F)c(F)cc3F)ccnc2C2CCC(F)(F)CO2)cn1] | N-(2-(5,5-difluorotetrahydro-2H- pyran-2-yl)-4-(2,4,5- trifluorophenyl)pyridin-3-yl)-2- isopropylpyrimidine-5- carboxamide P1 |
| 372 | 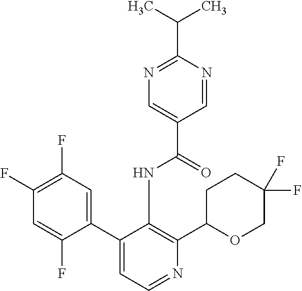 [SMILES: CC(C)c1ncc(C(=O)Nc2c(-c3cc(F)c(F)cc3F)ccnc2C2CCC(F)(F)CO2)cn1] | N-(2-(5,5-difluorotetrahydro-2H- pyran-2-yl)-4-(2,4,5- trifluorophenyl)pyridin-3-yl)-2- isopropylpyrimidine-5- carboxamide P2 |
| 373 | 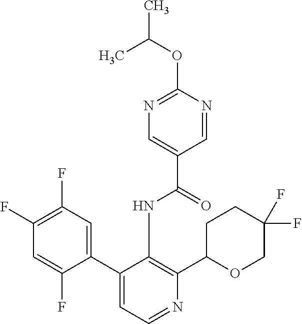 [SMILES: CC(C)Oc1ncc(C(=O)Nc2c(-c3cc(F)c(F)cc3F)ccnc2C2CCC(F)(F)CO2)cn1] | N-(2-(5,5-difluorotetrahydro-2H- pyran-2-yl)-4-(2,4,5- trifluorophenyl)pyridin-3-yl)-2- isopropoxypyrimidine-5- carboxamide P1 |
| 374 | 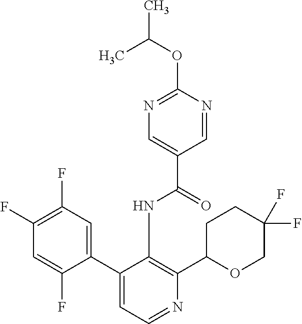 [SMILES: CC(C)Oc1ncc(C(=O)Nc2c(-c3cc(F)c(F)cc3F)ccnc2C2CCC(F)(F)CO2)cn1] | N-(2-(5,5-difluorotetrahydro-2H- pyran-2-yl)-4-(2,4,5- trifluorophenyl)pyridin-3-yl)-2- isopropoxypyrimidine-5- carboxamide P2 |
| 375 | 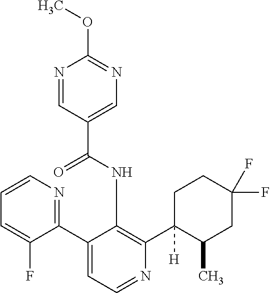 [SMILES: COc1ncc(C(=O)Nc2c(-c3ncccc3F)ccnc2[C@H]2CCC(F)(F)C[C@H]2C)cn1] | N-(2′-(syn)-4,4-difluoro-2- methylcyclohexyl)-3-fluoro-[2,4′- bipyridin]-3′-yl)-2- methoxypyrimidine-5- carboxamide P1 |
| 376 | 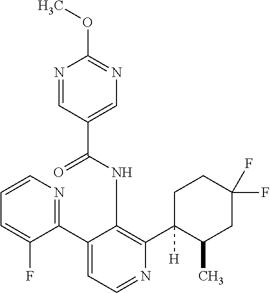 [SMILES: COc1ncc(C(=O)Nc2c(-c3ncccc3F)ccnc2[C@H]2CCC(F)(F)C[C@H]2C)cn1] | N-(2′-(syn)-4,4-difluoro-2- methylcyclohexyl)-3-fluoro-[2,4′- bipyridin]-3′-yl)-2- methoxypyrimidine-5- carboxamide P2 |
| 377 | 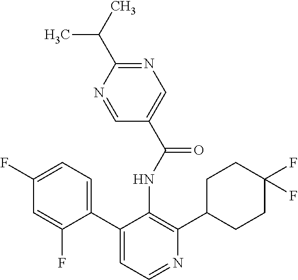 [SMILES: CC(C)c1ncc(C(=O)Nc2c(-c3ccc(F)cc3F)ccnc2C2CCC(F)(F)CC2)cn1] | N-(2-(4,4-difluorocyclohexyl)-4- (2,4-difluorophenyl)pyridin-3-yl)- 2-isopropylpyrimidine-5- carboxamide |
| 378 | 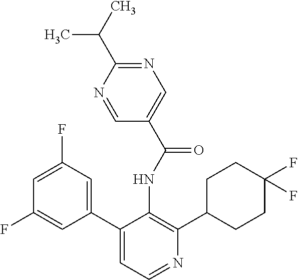 [SMILES: CC(C)c1ncc(C(=O)Nc2c(-c3cc(F)cc(F)c3)ccnc2C2CCC(F)(F)CC2)cn1] | N-[2-(4,4-difluorocyclohexyl)-4- (3,5-difluorophenyl)-3-pyridyl]-2- isopropyl-pyrimidine-5- carboxamide |
| 379 | 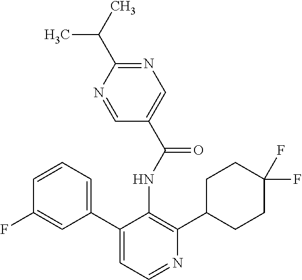 [SMILES: CC(C)c1ncc(C(=O)Nc2c(-c3cccc(F)c3)ccnc2C2CCC(F)(F)CC2)cn1] | N-(2-(4,4-difluorocyclohexyl)-4- (3-fluorophenyl)pyridin-3-yl)-2- isopropylpyrimidine-5- carboxamide |
| 380 | 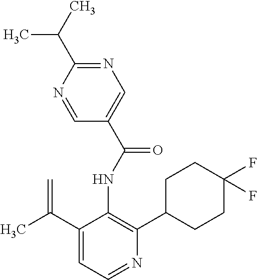 [SMILES: C=C(C)c1ccnc(C2CCC(F)(F)CC2)c1NC(=O)c1cnc(C(C)C)nc1] | N-(2-(4,4-difluorocyclohexyl)-4- (prop-1-en-2-yl)pyridin-3-yl)-2- isopropylpyrimidine-5- carboxamide |
| 381 | 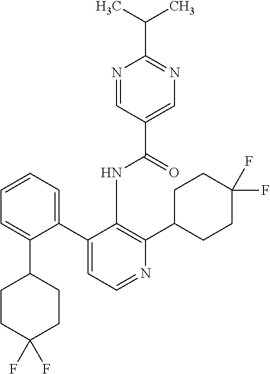 [SMILES: CC(C)c1ncc(C(=O)Nc2c(-c3ccccc3C3CCC(F)(F)CC3)ccnc2C2CCC(F)(F)CC2)cn1] | N-(2-(4,4-difluorocyclohexyl)-4- (2-(4,4- difluorocyclohexyl)phenyl)pyridin- 3-yl)-2-isopropylpyrimidine-5- carboxamide |
| 382 | 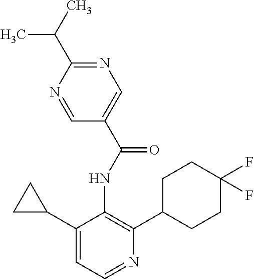 [SMILES: CC(C)c1ncc(C(=O)Nc2c(C3CC3)ccnc2C2CCC(F)(F)CC2)cn1] | N-(4-cyclopropyl-2-(4,4- difluorocyclohexyl)pyridin-3-yl)-2- isopropylpyrimidine-5- carboxamide |
| 383 | 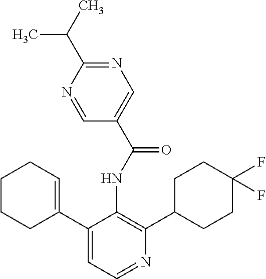 [SMILES: CC(C)c1ncc(C(=O)Nc2c(C3=CCCCC3)ccnc2C2CCC(F)(F)CC2)cn1] | N-(4-(cyclohex-1-en-1-yl)-2-(4,4- difluorocyclohexyl)pyridin-3-yl)-2- isopropylpyrimidine-5- carboxamide |
| 384 | 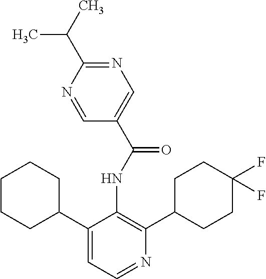 [SMILES: CC(C)c1ncc(C(=O)Nc2c(C3CCCCC3)ccnc2C2CCC(F)(F)CC2)cn1] | N-(4-cyclohexyl-2-(4,4- difluorocyclohexyl)pyridin-3-yl)-2- isopropylpyrimidine-5- carboxamide |
| 385 | 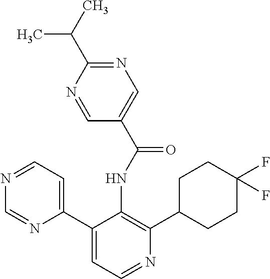 [SMILES: CC(C)c1ncc(C(=O)Nc2c(-c3ccncn3)ccnc2C2CCC(F)(F)CC2)cn1] | N-(2-(4,4-difluorocyclohexyl)-4- (pyrimidin-4-yl)pyridin-3-yl)-2- isopropylpyrimidine-5- carboxamide |
| 386 | 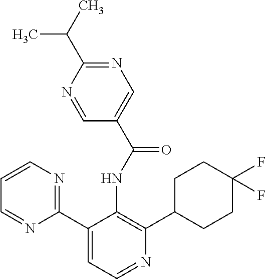 [SMILES: CC(C)c1ncc(C(=O)Nc2c(-c3ncccn3)ccnc2C2CCC(F)(F)CC2)cn1] | N-(2-(4,4-difluorocyclohexyl)-4- (pyrimidin-2-yl)pyridin-3-yl)-2- isopropylpyrimidine-5- carboxamide |
| 387 | 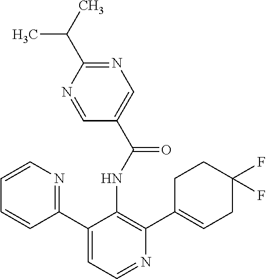 [SMILES: CC(C)c1ncc(C(=O)Nc2c(-c3ccccn3)ccnc2C2=CCC(F)(F)CC2)cn1] | N-(2′-(4,4-difluorocyclohex-1-en- 1-yl)-[2,4′-bipyridin]-3′-yl)-2- isopropylpyrimidine-5- carboxamide |
| 388 | 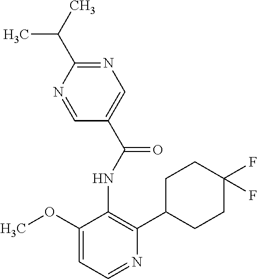 [SMILES: COc1ccnc(C2CCC(F)(F)CC2)c1NC(=O)c1cnc(C(C)C)nc1] | N-(2-(4,4-difluorocyclohexyl)-4- methoxypyridin-3-yl)-2- isopropylpyrimidine-5- carboxamide |
| 389 | 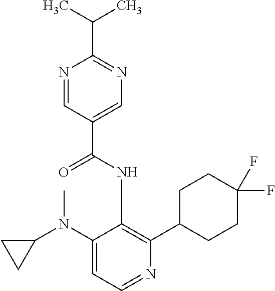 [SMILES: CC(C)c1ncc(C(=O)Nc2c(N(C)C3CC3)ccnc2C2CCC(F)(F)CC2)cn1] | N-(4-(cyclopropyl(methyl)amino)- 2-(4,4-difluorocyclohexyl)pyridin- 3-yl)-2-isopropylpyrimidine-5- carboxamide |
| 390 | 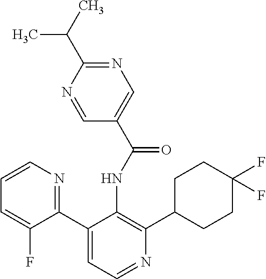 [SMILES: CC(C)c1ncc(C(=O)Nc2c(-c3ncccc3F)ccnc2C2CCC(F)(F)CC2)cn1] | N-(2′-(4,4-difluorocyclohexyl)-3- fluoro-[2,4′-bipyridin]-3′-yl)-2- isopropylpyrimidine-5- carboxamide |
| 391 | 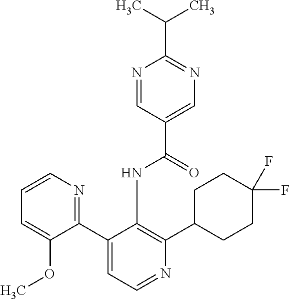 [SMILES: COc1cccnc1-c1ccnc(C2CCC(F)(F)CC2)c1NC(=O)c1cnc(C(C)C)nc1] | N-(2′-(4,4-difluorocyclohexyl)-3- methoxy-[2,4′-bipyridin]-3′-yl)-2- isopropylpyrimidine-5- carboxamide |
| 392 | 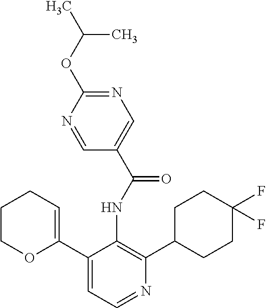 [SMILES: CC(C)Oc1ncc(C(=O)Nc2c(C3=CCCCO3)ccnc2C2CCC(F)(F)CC2)cn1] | N-(2-(4,4-difluorocyclohexyl)-4- (3,4-dihydro-2H-pyran-6- yl)pyridin-3-yl)-2- isopropoxypyrimidine-5- carboxamide |
| 393 | 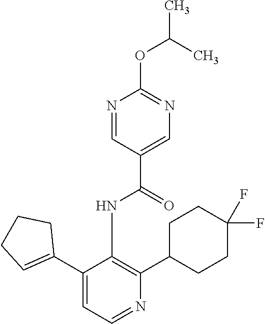 [SMILES: CC(C)Oc1ncc(C(=O)Nc2c(C3=CCCC3)ccnc2C2CCC(F)(F)CC2)cn1] | N-(4-(cyclopent-1-en-1-yl)-2- (4,4-difluorocyclohexyl)pyridin-3- yl)-2-isopropoxypyrimidine-5- carboxamide |
| 394 | 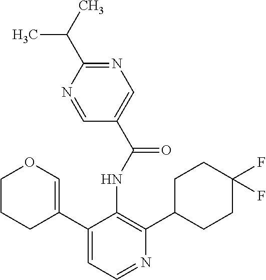 [SMILES: CC(C)c1ncc(C(=O)Nc2c(C3=COCCC3)ccnc2C2CCC(F)(F)CC2)cn1] | N-(2-(4,4-difluorocyclohexyl)-4- (3,4-dihydro-2H-pyran-5- yl)pyridin-3-yl)-2- isopropylpyrimidine-5- carboxamide |
| 395 | 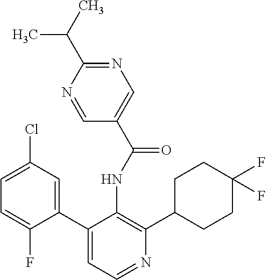 [SMILES: CC(C)c1ncc(C(=O)Nc2c(-c3cc(Cl)ccc3F)ccnc2C2CCC(F)(F)CC2)cn1] | N-(4-(5-chloro-2-fluorophenyl)-2- (4,4-difluorocyclohexyl)pyridin-3- yl)-2-isopropylpyrimidine-5- carboxamide |
| 396 | 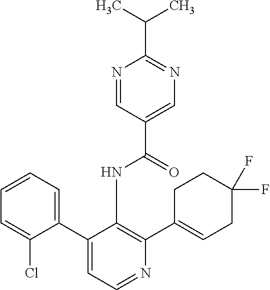 [SMILES: CC(C)c1ncc(C(=O)Nc2c(-c3ccccc3Cl)ccnc2C2=CCC(F)(F)CC2)cn1] | N-(4-(2-chlorophenyl)-2-(4,4- difluorocyclohex-1-en-1- yl)pyridin-3-yl)-2- isopropylpyrimidine-5- carboxamide |
| 397 | 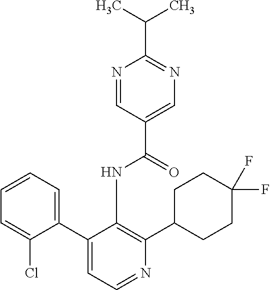 [SMILES: CC(C)c1ncc(C(=O)Nc2c(-c3ccccc3Cl)ccnc2C2CCC(F)(F)CC2)cn1] | N-(4-(2-chlorophenyl)-2-(4,4- difluorocyclohexyl)pyridin-3-yl)-2- isopropylpyrimidine-5- carboxamide |
| 398 | 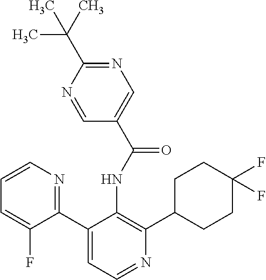 [SMILES: CC(C)(C)c1ncc(C(=O)Nc2c(-c3ncccc3F)ccnc2C2CCC(F)(F)CC2)cn1] | 2-(tert-butyl)-N-(2′-(4,4- difluorocyclohexyl)-3-fluoro-[2,4′- bipyridin]-3′-yl)pyrimidine-5- carboxamide |
| 399 | 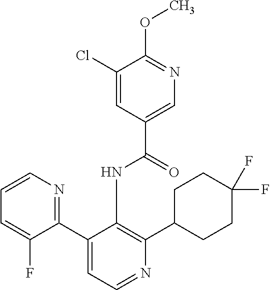 [SMILES: COc1ncc(C(=O)Nc2c(-c3ncccc3F)ccnc2C2CCC(F)(F)CC2)cc1Cl] | 5-chloro-N-(2′-(4,4- difluorocyclohexyl)-3-fluoro-[2,4′- bipyridin]-3′-yl)-6- methoxynicotinamide |
| 400 | 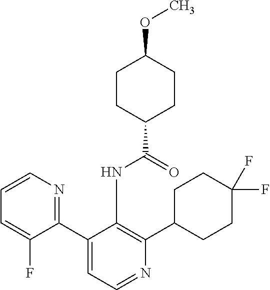 [SMILES: CO[C@H]1CC[C@H](C(=O)Nc2c(-c3ncccc3F)ccnc2C2CCC(F)(F)CC2)CC1] | (anti)-N-(2-(4,4- difluorocyclohexyl)-3-fluoro-[2,4′- bipyridin]-3′-yl)-4- methoxycyclohexane-1- carboxamide |
| 401 | 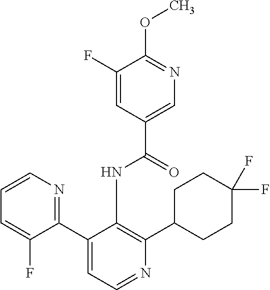 [SMILES: COc1ncc(C(=O)Nc2c(-c3ncccc3F)ccnc2C2CCC(F)(F)CC2)cc1F] | N-(2′-(4,4-difluorocyclohexyl)-3- fluoro-[2,4′-bipyridin]-3′-yl)-5- fluoro-6-methoxynicotinamide |
| 402 | 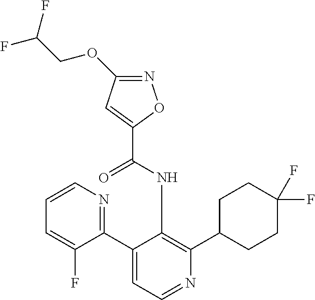 [SMILES: O=C(Nc1c(-c2ncccc2F)ccnc1C1CCC(F)(F)CC1)c1cc(OCC(F)F)no1] | N-(2′-(4,4-difluorocyclohexyl)-3- fluoro-[2,4′-bipyridin]-3′-yl)-3- (2,2-difluoroethoxy)isoxazole-5- carboxamide |
| 403 | 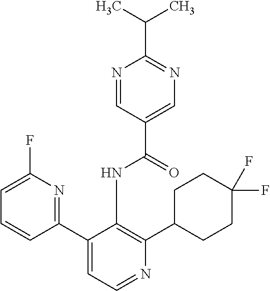 [SMILES: CC(C)c1ncc(C(=O)Nc2c(-c3cccc(F)n3)ccnc2C2CCC(F)(F)CC2)cn1] | N-(2′-(4,4-difluorocyclohexyl)-6- fluoro-[2,4′-bipyridin]-3′-yl)-2- isopropylpyrimidine-5- carboxamide |
| 404 | 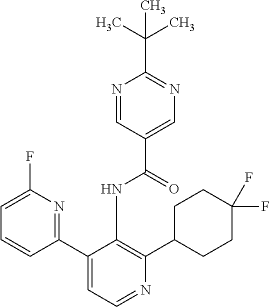 [SMILES: CC(C)(C)c1ncc(C(=O)Nc2c(-c3cccc(F)n3)ccnc2C2CCC(F)(F)CC2)cn1] | 2-(tert-butyl)-N-(2′-(4,4- difluorocyclohexyl)-6-fluoro-[2,4′- bipyridin]-3′-yl)pyrimidine-5- carboxamide |
| 405 | 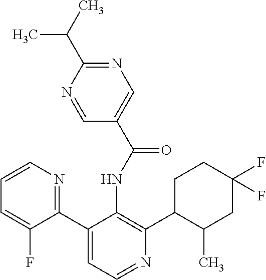 [SMILES: CC(C)c1ncc(C(=O)Nc2c(-c3ncccc3F)ccnc2C2CCC(F)(F)CC2C)cn1] | N-(2′-(4,4-difluoro-2- methylcyclohexyl)-3-fluoro-[2,4′- bipyridin]-3′-yl)-2- isopropylpyrimidine-5- carboxamide |
| 406 | 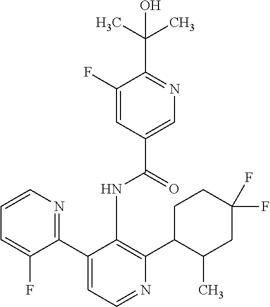 [SMILES: CC1CC(F)(F)CCC1c1nccc(-c2ncccc2F)c1NC(=O)c1cnc(C(C)(C)O)c(F)c1] | N-(2′-(4,4-difluoro-2- methylcyclohexyl)-3-fluoro-[2,4′- bipyridin]-3′-yl)-5-fluoro-6-(2- hydroxypropan-2-yl)nicotinamide D1 |
| 407 | 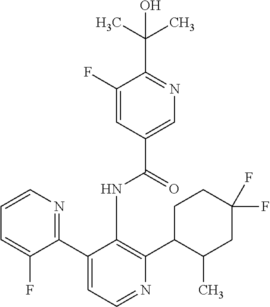 [SMILES: CC1CC(F)(F)CCC1c1nccc(-c2ncccc2F)c1NC(=O)c1cnc(C(C)(C)O)c(F)c1] | N-(2′-(4,4-difluoro-2- methylcyclohexyl)-3-fluoro-[2,4′- bipyridin]-3′-yl)-5-fluoro-6-(2- hydroxypropan-2-yl)nicotinamide D2 |
| 408 | 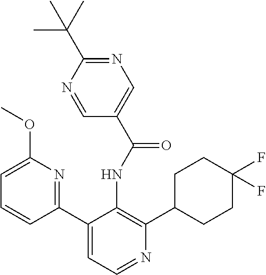 [SMILES: COc1cccc(-c2ccnc(C3CCC(F)(F)CC3)c2NC(=O)c2cnc(C(C)(C)C)nc2)n1] | 2-(tert-butyl)-N-(2′-(4,4- difluorocyclohexyl)-6-methoxy- [2,4′-bipyridin]-3′-yl)pyrimidine-5- carboxamide |
| 409 | 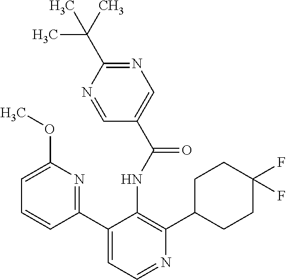 [SMILES: COc1cccc(-c2ccnc(C3CCC(F)(F)CC3)c2NC(=O)c2cnc(C(C)(C)C)nc2)n1] | 6-(3,3-difluorocyclobutoxy)-N-(2′- (5,5-difluorotetrahydro-2H-pyran- 2-yl)-3-fluoro-[2,4′-bipyridin]-3′- yl)-5-fluoronicotinamide P1 |
| 410 | 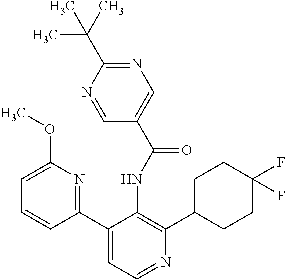 [SMILES: COc1cccc(-c2ccnc(C3CCC(F)(F)CC3)c2NC(=O)c2cnc(C(C)(C)C)nc2)n1] | 6-(3,3-difluorocyclobutoxy)-N-(2′- (5,5-difluorotetrahydro-2H-pyran- 2-yl)-3-fluoro-[2,4′-bipyridin]-3′- yl)-5-fluoronicotinamide P2 |
| 411 | 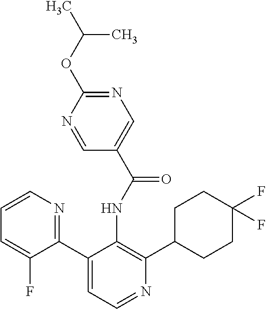 [SMILES: CC(C)Oc1ncc(C(=O)Nc2c(-c3ncccc3F)ccnc2C2CCC(F)(F)CC2)cn1] | N-(2′-(4,4-difluorocyclohexyl)-3- fluoro-[2,4′-bipyridin]-3′-yl)-2- isopropoxypyrimidine-5- carboxamide |
| 412 | 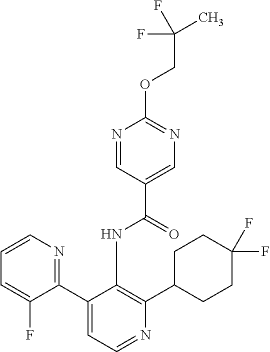 [SMILES: CC(F)(F)COc1ncc(C(=O)Nc2c(-c3ncccc3F)ccnc2C2CCC(F)(F)CC2)cn1] | N-(2′-(4,4-difluorocyclohexyl)-3- fluoro-[2,4′-bipyridin]-3′-yl)-2- (2,2-difluoropropoxy)pyrimidine- 5-carboxamide |
| 413 | 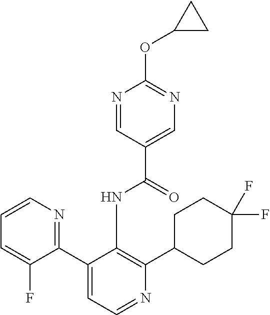 [SMILES: O=C(Nc1c(-c2ncccc2F)ccnc1C1CCC(F)(F)CC1)c1cnc(OC2CC2)nc1] | 2-cyclopropoxy-N-(2′-(4,4- difluorocyclohexyl)-3-fluoro-[2,4′- bipyridin]-3′-yl)pyrimidine-5- carboxamide |
| 414 | 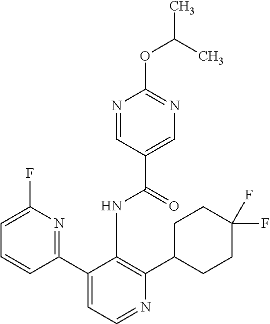 [SMILES: CC(C)Oc1ncc(C(=O)Nc2c(-c3cccc(F)n3)ccnc2C2CCC(F)(F)CC2)cn1] | N-(2′-(4,4-difluorocyclohexyl)-6- fluoro-[2,4′-bipyridin]-3′-yl)-2- isopropoxypyrimidine-5- carboxamide |
| 415 | 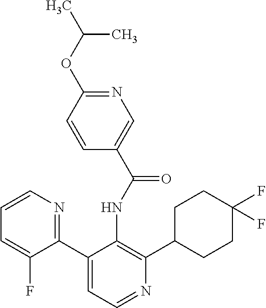 [SMILES: CC(C)Oc1ccc(C(=O)Nc2c(-c3ncccc3F)ccnc2C2CCC(F)(F)CC2)cn1] | N-(2′-(4,4-difluorocyclohexyl)-3- fluoro-[2,4′-bipyridin]-3′-yl)-6- isopropoxynicotinamide |
| 416 | 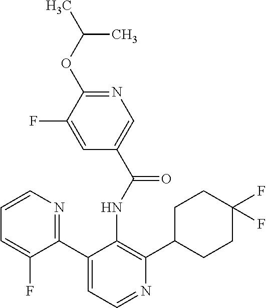 [SMILES: CC(C)Oc1ncc(C(=O)Nc2c(-c3ncccc3F)ccnc2C2CCC(F)(F)CC2)cc1F] | N-(2′-(4,4-difluorocyclohexyl)-3- fluoro-[2,4′-bipyridin]-3′-yl)-5- fluoro-6-isopropoxynicotinamide |
| 417 | 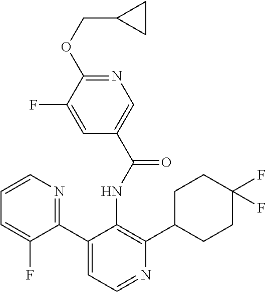 [SMILES: O=C(Nc1c(-c2ncccc2F)ccnc1C1CCC(F)(F)CC1)c1cnc(OCC2CC2)c(F)c1] | 6-(cyclopropylmethoxy)-N-(2′- (4,4-difluorocyclohexyl)-3-fluoro- [2,4′-bipyridin]-3′-yl)-5- fluoronicotinamide |
| 418 | 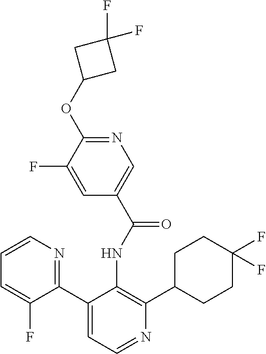 [SMILES: O=C(Nc1c(-c2ncccc2F)ccnc1C1CCC(F)(F)CC1)c1cnc(OC2CC(F)(F)C2)c(F)c1] | 6-(3,3-difluorocyclobutoxy)-N-(2′- (4,4-difluorocyclohexyl)-3-fluoro- [2,4′-bipyridin]-3′-yl)-5- fluoronicotinamide |
| 419 | 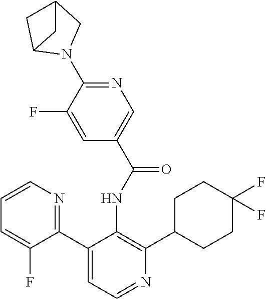 [SMILES: O=C(Nc1c(-c2ncccc2F)ccnc1C1CCC(F)(F)CC1)c1cnc(N2CC3CC2C3)c(F)c1] | 6-(2-azabicyclo[2.1.1]hexan-2- yl)-N-(2′-(4,4-difluorocyclohexyl)- 3-fluoro-[2,4′-bipyridin]-3′-yl)-5- fluoronicotinamide |
| 420 | 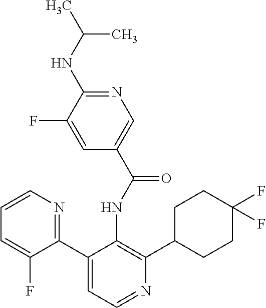 [SMILES: CC(C)Nc1ncc(C(=O)Nc2c(-c3ncccc3F)ccnc2C2CCC(F)(F)CC2)cc1F] | N-(2′-(4,4-difluorocyclohexyl)-3- fluoro-[2,4′-bipyridin]-3′-yl)-5- fluoro-6- (isopropylamino)nicotinamide |
| 421 | 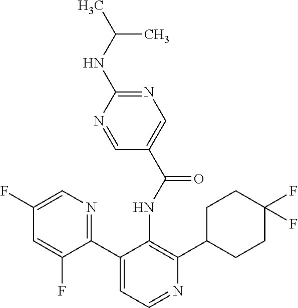 [SMILES: CC(C)Nc1ncc(C(=O)Nc2c(-c3ncc(F)cc3F)ccnc2C2CCC(F)(F)CC2)cn1] | N-(2′-(4,4-difluorocyclohexyl)- 3,5-difluoro-[2,4′-bipyridin]-3′-yl)- 2-isopropoxypyrimidine-5- carboxamide |
| 422 | 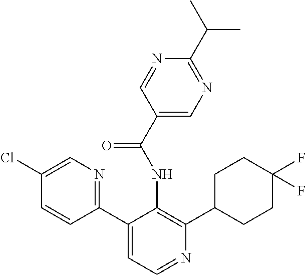 [SMILES: CC(C)c1ncc(C(=O)Nc2c(-c3ccc(Cl)cn3)ccnc2C2CCC(F)(F)CC2)cn1] | N-(5-chloro-2′-(4,4- difluorocyclohexyl)-[2,4′- bipyridin]-3′-yl)-2- isopropylpyrimidine-5- carboxamide |
| 423 | 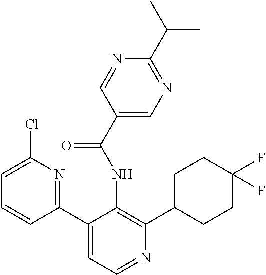 [SMILES: CC(C)c1ncc(C(=O)Nc2c(-c3cccc(Cl)n3)ccnc2C2CCC(F)(F)CC2)cn1] | N-(6-chloro-2′-(4,4- difluorocyclohexyl)-[2,4′- bipyridin]-3′-yl)-2- isopropylpyrimidine-5- carboxamide |
| 424 | 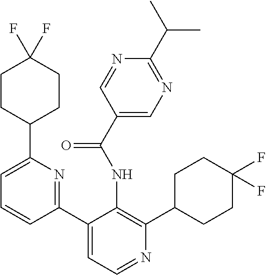 [SMILES: CC(C)c1ncc(C(=O)Nc2c(-c3cccc(C4CCC(F)(F)CC4)n3)ccnc2C2CCC(F)(F)CC2)cn1] | N-(2′,6-bis(4,4- difluorocyclohexyl)-[2,4′- bipyridin]-3′-yl)-2- isopropylpyrimidine-5- carboxamide |
| 425 | 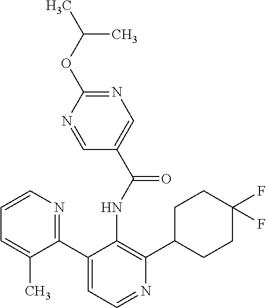 [SMILES: Cc1cccnc1-c1ccnc(C2CCC(F)(F)CC2)c1NC(=O)c1cnc(OC(C)C)nc1] | N-(2′-(4,4-difluorocyclohexyl)-3- methyl-[2,4′-bipyridin]-3′-yl)-2- isopropoxypyrimidine-5- carboxamide |
| 426 | 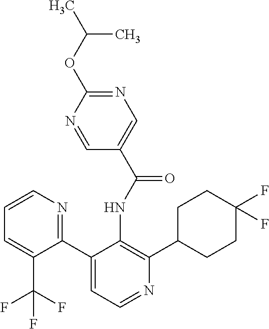 [SMILES: CC(C)Oc1ncc(C(=O)Nc2c(-c3ncccc3C(F)(F)F)ccnc2C2CCC(F)(F)CC2)cn1] | N-(2′-(4,4-difluorocyclohexyl)-3- (trifluoromethyl)-[2,4′-bipyridin]- 3′-yl)-2-isopropoxypyrimidine-5- carboxamide |
| 427 | 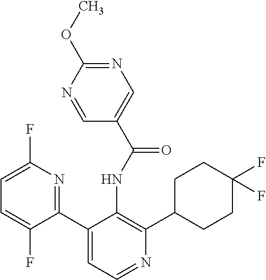 [SMILES: COc1ncc(C(=O)Nc2c(-c3nc(F)ccc3F)ccnc2C2CCC(F)(F)CC2)cn1] | N-(2′-(4,4-difluorocyclohexyl)- 3,6-difluoro-[2,4′-bipyridin]-3′-yl)- 2-methoxypyrimidine-5- carboxamide |
| 428 | 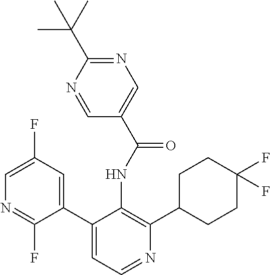 [SMILES: CC(C)(C)c1ncc(C(=O)Nc2c(-c3cc(F)cnc3F)ccnc2C2CCC(F)(F)CC2)cn1] | 2-(tert-butyl)-N-(2′-(4,4- difluorocyclohexyl)-2,5-difluoro- [3,4′-bipyridin]-3′-yl)pyrimidine-5- carboxamide |
| 429 | 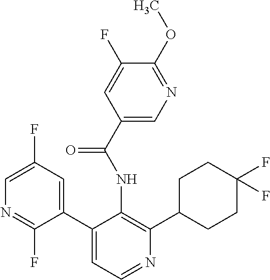 [SMILES: COc1ncc(C(=O)Nc2c(-c3cc(F)cnc3F)ccnc2C2CCC(F)(F)CC2)cc1F] | N-(2′-(4,4-difluorocyclohexyl)- 2,5-difluoro-[3,4′-bipyridin]-3′-yl)- 5-fluoro-6-methoxynicotinamide |
| 430 | 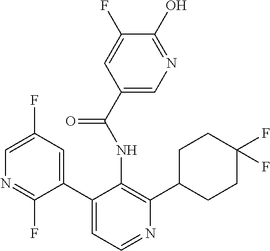 [SMILES: O=C(Nc1c(-c2cc(F)cnc2F)ccnc1C1CCC(F)(F)CC1)c1cnc(O)c(F)c1] | N-(2′-(4,4-difluorocyclohexyl)- 2,5-difluoro-[3,4′-bipyridin]-3′-yl)- 5-fluoro-6-hydroxynicotinamide |
| 431 | 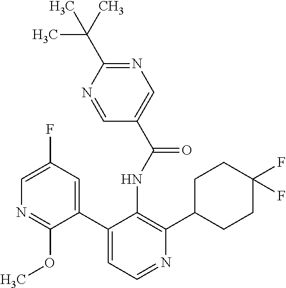 [SMILES: COc1ncc(F)cc1-c1ccnc(C2CCC(F)(F)CC2)c1NC(=O)c1cnc(C(C)(C)C)nc1] | 2-(tert-butyl)-N-(2′-(4,4- difluorocyclohexyl)-5-fluoro-2- methoxy-[3,4′-bipyridin]-3′- yl)pyrimidine-5-carboxamide |
| 432 | 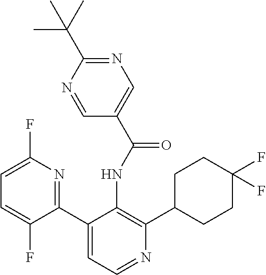 [SMILES: CC(C)(C)c1ncc(C(=O)Nc2c(-c3nc(F)ccc3F)ccnc2C2CCC(F)(F)CC2)cn1] | 2-(tert-butyl)-N-(2′-(4,4- difluorocyclohexyl)-3,6-difluoro- [2,4′-bipyridin]-3′-yl)pyrimidine-5- carboxamide |
| 433 | 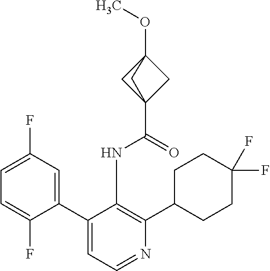 [SMILES: COC12CC(C(=O)Nc3c(-c4cc(F)ccc4F)ccnc3C3CCC(F)(F)CC3)(C1)C2] | N-(2-(4,4-difluorocyclohexyl)-4- (2,5-difluorophenyl)pyridin-3-yl)- 3-methoxybicyclo[1.1.1]pentane- 1-carboxamide |
| 434 | 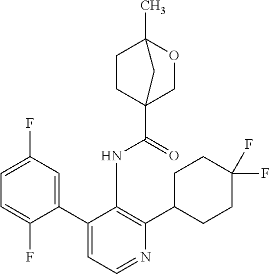 [SMILES: CC12CCC(C(=O)Nc3c(-c4cc(F)ccc4F)ccnc3C3CCC(F)(F)CC3)(CO1)C2] | N-(2-(4,4-difluorocyclohexyl)-4- (2,5-difluorophenyl)pyridin-3-yl)- 1-methyl-2- oxabicyclo[2.2.1]heptane-4- carboxamide |
| 435 | 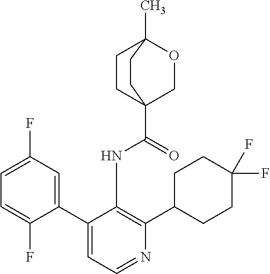 [SMILES: CC12CCC(C(=O)Nc3c(-c4cc(F)ccc4F)ccnc3C3CCC(F)(F)CC3)(CC1)CO2] | N-(2-(4,4-difluorocyclohexyl)-4- (2,5-difluorophenyl)pyridin-3-yl)- 1-methyl-2- oxabicyclo[2.2.2]octane-4- carboxamide |
| 436 | 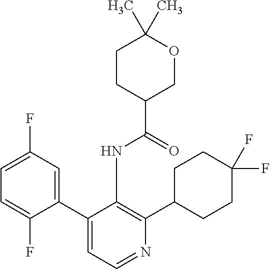 [SMILES: CC1(C)CCC(C(=O)Nc2c(-c3cc(F)ccc3F)ccnc2C2CCC(F)(F)CC2)CO1] | N-(2-(4,4-difluorocyclohexyl)-4- (2,5-difluorophenyl)pyridin-3-yl)- 6,6-dimethyltetrahydro-2H- pyran-3-carboxamide |
| 437 | 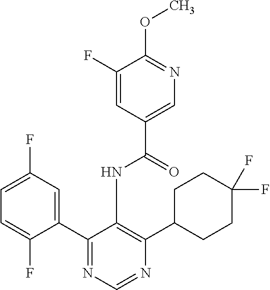 [SMILES: COc1ncc(C(=O)Nc2c(-c3cc(F)ccc3F)ncnc2C2CCC(F)(F)CC2)cc1F] | N-(4-(4,4-difluorocyclohexyl)-6- (2,5-difluorophenyl)pyrimidin-5- yl)-5-fluoro-6- methoxynicotinamide |
| 438 | 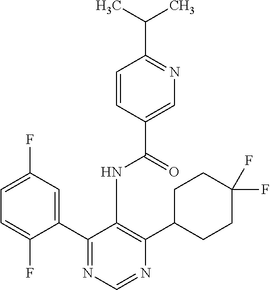 [SMILES: CC(C)c1ccc(C(=O)Nc2c(-c3cc(F)ccc3F)ncnc2C2CCC(F)(F)CC2)cn1] | N-(4-(4,4-difluorocyclohexyl)-6- (2,5-difluorophenyl)pyrimidin-5- yl)-6-isopropylnicotinamide |
| 439 | 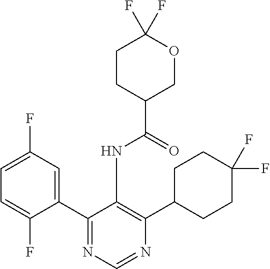 [SMILES: O=C(Nc1c(-c2cc(F)ccc2F)ncnc1C1CCC(F)(F)CC1)C1CCC(F)(F)OC1] | N-(4-(4,4-difluorocyclohexyl)-6- (2,5-difluorophenyl)pyrimidin-5- yl)-6,6-difluorotetrahydro-2H- pyran-3-carboxamide |
| 440 | 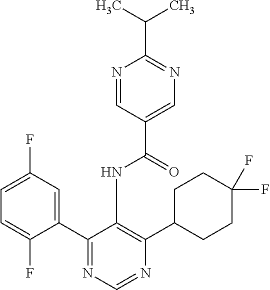 [SMILES: CC(C)c1ncc(C(=O)Nc2c(-c3cc(F)ccc3F)ncnc2C2CCC(F)(F)CC2)cn1] | N-(4-(4,4-difluorocyclohexyl)-6- (2,5-difluorophenyl)pyrimidin-5- yl)-2-isopropylpyrimidine-5- carboxamide |
| 441 | 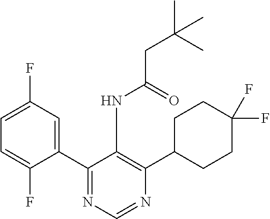 [SMILES: CC(C)(C)CC(=O)Nc1c(-c2cc(F)ccc2F)ncnc1C1CCC(F)(F)CC1] | N-(4-(4,4-difluorocyclohexyl)-6- (2,5-difluorophenyl)pyrimidin-5- yl)-3,3-dimethylbutanamide |
| 442 | 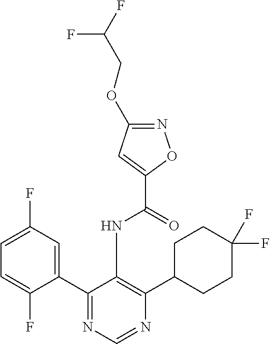 [SMILES: O=C(Nc1c(-c2cc(F)ccc2F)ncnc1C1CCC(F)(F)CC1)c1cc(OCC(F)F)no1] | N-(4-(4,4-difluorocyclohexyl)-6- (2,5-difluorophenyl)pyrimidin-5- yl)-3-(2,2- difluoroethoxy)isoxazole-5- carboxamide |
| 443 | 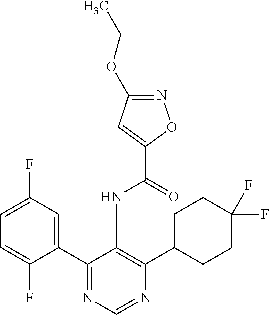 [SMILES: CCOc1cc(C(=O)Nc2c(-c3cc(F)ccc3F)ncnc2C2CCC(F)(F)CC2)on1] | N-(4-(4,4-difluorocyclohexyl)-6- (2,5-difluorophenyl)pyrimidin-5- yl)-3-ethoxyisoxazole-5- carboxamide |
| 444 | 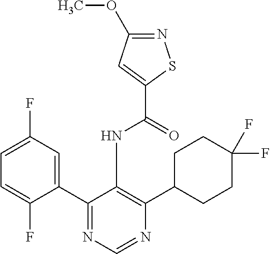 [SMILES: COc1cc(C(=O)Nc2c(-c3cc(F)ccc3F)ncnc2C2CCC(F)(F)CC2)sn1] | N-(4-(4,4-difluorocyclohexyl)-6- (2,5-difluorophenyl)pyrimidin-5- yl)-3-methoxyisothiazole-5- carboxamide |
| 445 | 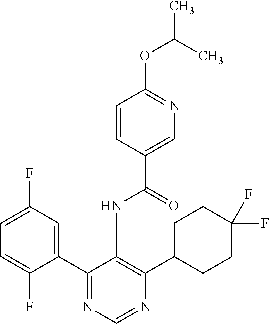 [SMILES: CC(C)Oc1ccc(C(=O)Nc2c(-c3cc(F)ccc3F)ncnc2C2CCC(F)(F)CC2)cn1] | N-(4-(4,4-difluorocyclohexyl)-6- (2,5-difluorophenyl)pyrimidin-5- yl)-6-isopropoxynicotinamide |
| 446 | 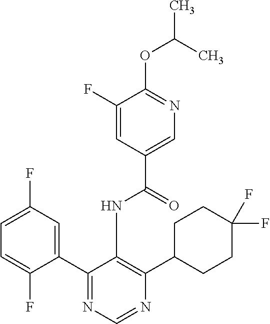 [SMILES: CC(C)Oc1ncc(C(=O)Nc2c(-c3cc(F)ccc3F)ncnc2C2CCC(F)(F)CC2)cc1F] | N-(4-(4,4-difluorocyclohexyl)-6- (2,5-difluorophenyl)pyrimidin-5- yl)-5-fluoro-6- isopropoxynicotinamide |
| 447 | 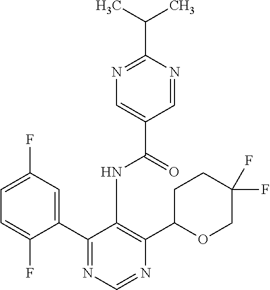 [SMILES: CC(C)c1ncc(C(=O)Nc2c(-c3cc(F)ccc3F)ncnc2C2CCC(F)(F)CO2)cn1] | N-(4-(2,5-difluorophenyl)-6-(5,5- difluorotetrahydro-2H-pyran-2- yl)pyrimidin-5-yl)-2- isopropylpyrimidine-5- carboxamide P1 |
| 448 | 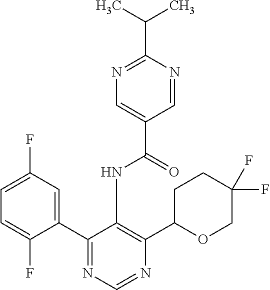 [SMILES: CC(C)c1ncc(C(=O)Nc2c(-c3cc(F)ccc3F)ncnc2C2CCC(F)(F)CO2)cn1] | N-(4-(2,5-difluorophenyl)-6-(5,5- difluorotetrahydro-2H-pyran-2- yl)pyrimidin-5-yl)-2- isopropylpyrimidine-5- carboxamide P2 |
| 449 | 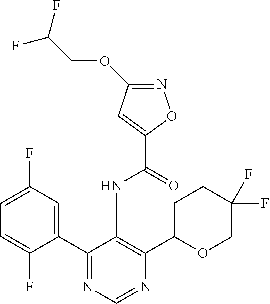 [SMILES: O=C(Nc1c(-c2cc(F)ccc2F)ncnc1C1CCC(F)(F)CO1)c1cc(OCC(F)F)no1] | 3-(2,2-difluoroethoxy)-N-(4-(2,5- difluorophenyl)-6-(5,5- difluorotetrahydro-2H-pyran-2- yl)pyrimidin-5-yl)isoxazole-5- carboxamide |
| 450 | 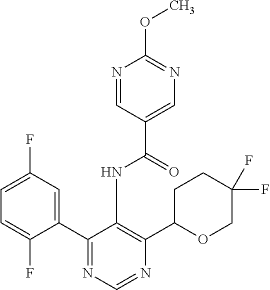 [SMILES: COc1ncc(C(=O)Nc2c(-c3cc(F)ccc3F)ncnc2C2CCC(F)(F)CO2)cn1] | N-(4-(2,5-difluorophenyl)-6-(5,5- difluorotetrahydro-2H-pyran-2- yl)pyrimidin-5-yl)-2- methoxypyrimidine-5- carboxamide |
| 451 | 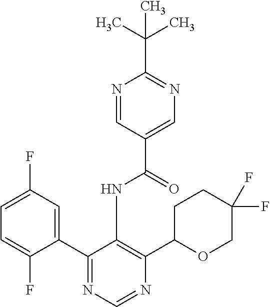 [SMILES: CC(C)(C)c1ncc(C(=O)Nc2c(-c3cc(F)ccc3F)ncnc2C2CCC(F)(F)CO2)cn1] | 2-(tert-butyl)-N-(4-(2,5- difluorophenyl)-6-(5,5- difluorotetrahydro-2H-pyran-2- yl)pyrimidin-5-yl)pyrimidine-5- carboxamide |
| 452 | 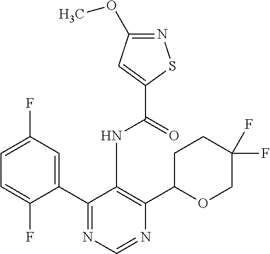 [SMILES: COc1cc(C(=O)Nc2c(-c3cc(F)ccc3F)ncnc2C2CCC(F)(F)CO2)sn1] | N-(4-(2,5-difluorophenyl)-6-(5,5- difluorotetrahydro-2H-pyran-2- yl)pyrimidin-5-yl)-3- methoxyisothiazole-5- carboxamide |
| 453 | 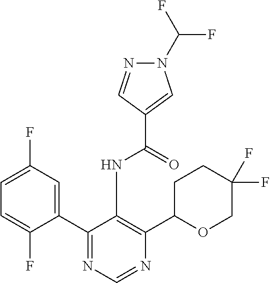 [SMILES: O=C(Nc1c(-c2cc(F)ccc2F)ncnc1C1CCC(F)(F)CO1)c1cnn(C(F)F)c1] | 1-(difluoromethyl)-N-(4-(2,5- difluorophenyl)-6-(5,5- difluorotetrahydro-2H-pyran-2- yl)pyrimidin-5-yl)-1H-pyrazole-4- carboxamide |
| 454 | 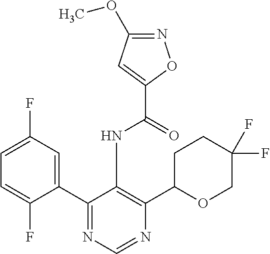 [SMILES: COc1cc(C(=O)Nc2c(-c3cc(F)ccc3F)ncnc2C2CCC(F)(F)CO2)on1] | N-(4-(2,5-difluorophenyl)-6-(5,5- difluorotetrahydro-2H-pyran-2- yl)pyrimidin-5-yl)-3- methoxyisoxazole-5- carboxamide |
| 455 | 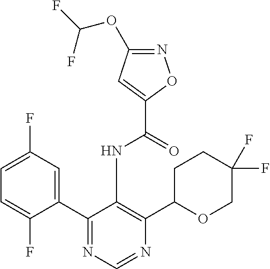 [SMILES: O=C(Nc1c(-c2cc(F)ccc2F)ncnc1C1CCC(F)(F)CO1)c1cc(OC(F)F)no1] | 3-(difluoromethoxy)-N-(4-(2,5- difluorophenyl)-6-(5,5- difluorotetrahydro-2H-pyran-2- yl)pyrimidin-5-yl)isoxazole-5- carboxamide |
| 456 | 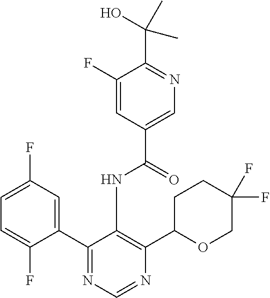 [SMILES: CC(C)(O)c1ncc(C(=O)Nc2c(-c3cc(F)ccc3F)ncnc2C2CCC(F)(F)CO2)cc1F] | N-(4-(2,5-difluorophenyl)-6-(5,5- difluorotetrahydro-2H-pyran-2- yl)pyrimidin-5-yl)-5-fluoro-6-(2- hydroxypropan-2-yl)nicotinamide |
| 457 | 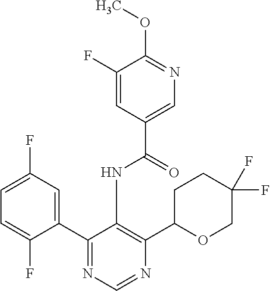 [SMILES: COc1ncc(C(=O)Nc2c(-c3cc(F)ccc3F)ncnc2C2CCC(F)(F)CO2)cc1F] | N-(4-(2,5-difluorophenyl)-6-(5,5- difluorotetrahydro-2H-pyran-2- yl)pyrimidin-5-yl)-5-fluoro-6- methoxynicotinamide |
| 458 | 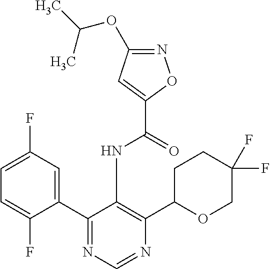 [SMILES: CC(C)Oc1cc(C(=O)Nc2c(-c3cc(F)ccc3F)ncnc2C2CCC(F)(F)CO2)on1] | N-(4-(2,5-difluorophenyl)-6-(5,5- difluorotetrahydro-2H-pyran-2- yl)pyrimidin-5-yl)-3- isopropoxyisoxazole-5- carboxamide |
| 459 | 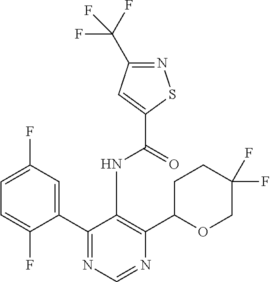 [SMILES: O=C(Nc1c(-c2cc(F)ccc2F)ncnc1C1CCC(F)(F)CO1)c1cc(C(F)(F)F)ns1] | N-(4-(2,5-difluorophenyl)-6-(5,5- difluorotetrahydro-2H-pyran-2- yl)pyrimidin-5-yl)-3- (trifluoromethyl)isothiazole-5- carboxamide |
| 460 | 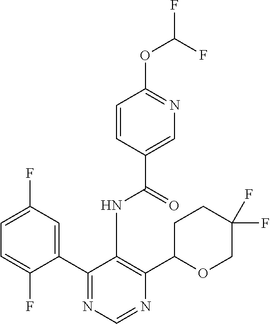 [SMILES: O=C(Nc1c(-c2cc(F)ccc2F)ncnc1C1CCC(F)(F)CO1)c1ccc(OC(F)F)nc1] | 6-(difluoromethoxy)-N-(4-(2,5- difluorophenyl)-6-(5,5- difluorotetrahydro-2H-pyran-2- yl)pyrimidin-5-yl)nicotinamide |
| 461 | 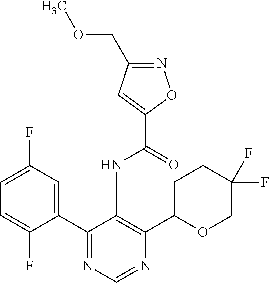 [SMILES: COCc1cc(C(=O)Nc2c(-c3cc(F)ccc3F)ncnc2C2CCC(F)(F)CO2)on1] | N-(4-(2,5-difluorophenyl)-6-(5,5- difluorotetrahydro-2H-pyran-2- yl)pyrimidin-5-yl)-3- (methoxymethyl)isoxazole-5- carboxamide |
| 462 | 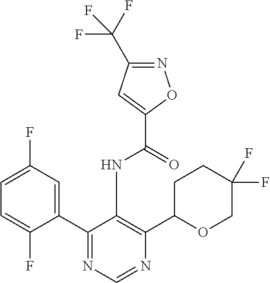 [SMILES: O=C(Nc1c(-c2cc(F)ccc2F)ncnc1C1CCC(F)(F)CO1)c1cc(C(F)(F)F)no1] | N-(4-(2,5-difluorophenyl)-6-(5,5- difluorotetrahydro-2H-pyran-2- yl)pyrimidin-5-yl)-3- (trifluoromethyl)isoxazole-5- carboxamide |
| 463 | 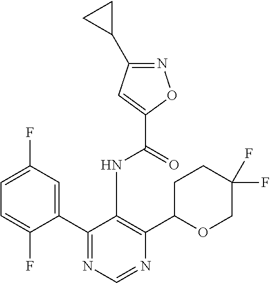 [SMILES: O=C(Nc1c(-c2cc(F)ccc2F)ncnc1C1CCC(F)(F)CO1)c1cc(C2CC2)no1] | 3-cyclopropyl-N-(4-(2,5- difluorophenyl)-6-(5,5- difluorotetrahydro-2H-pyran-2- yl)pyrimidin-5-yl)isoxazole-5- carboxamide |
| 464 | 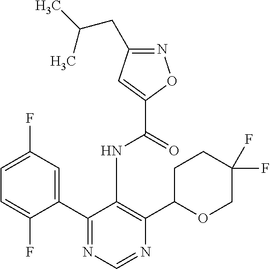 [SMILES: CC(C)Cc1cc(C(=O)Nc2c(-c3cc(F)ccc3F)ncnc2C2CCC(F)(F)CO2)on1] | N-(4-(2,5-difluorophenyl)-6-(5,5- difluorotetrahydro-2H-pyran-2- yl)pyrimidin-5-yl)-3- isobutylisoxazole-5-carboxamide |
| 465 | 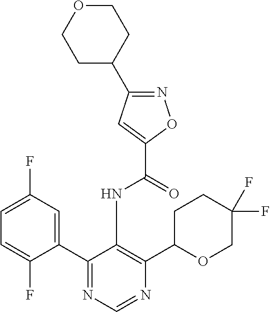 [SMILES: O=C(Nc1c(-c2cc(F)ccc2F)ncnc1C1CCC(F)(F)CO1)c1cc(C2CCOCC2)no1] | N-(4-(2,5-difluorophenyl)-6-(5,5- difluorotetrahydro-2H-pyran-2- yl)pyrimidin-5-yl)-3-(tetrahydro- 2H-pyran-4-yl)isoxazole-5- carboxamide |
| 466 | 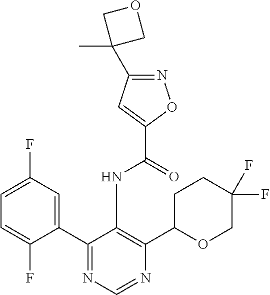 [SMILES: CC1(c2cc(C(=O)Nc3c(-c4cc(F)ccc4F)ncnc3C3CCC(F)(F)CO3)on2)COC1] | N-(4-(2,5-difluorophenyl)-6-(5,5- difluorotetrahydro-2H-pyran-2- yl)pyrimidin-5-yl)-3-(3- methyloxetan-3-yl)isoxazole-5- carboxamide |
| 467 | 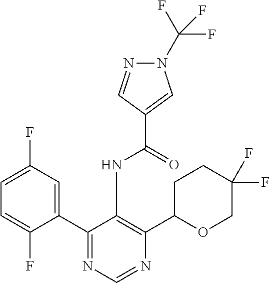 [SMILES: O=C(Nc1c(-c2cc(F)ccc2F)ncnc1C1CCC(F)(F)CO1)c1cnn(C(F)(F)F)c1] | N-(4-(2,5-difluorophenyl)-6-(5,5- difluorotetrahydro-2H-pyran-2- yl)pyrimidin-5-yl)-1- (trifluoromethyl)-1H-pyrazole-4- carboxamide |
| 468 | 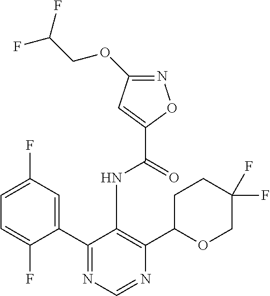 [SMILES: O=C(Nc1c(-c2cc(F)ccc2F)ncnc1C1CCC(F)(F)CO1)c1cc(OCC(F)F)no1] | 3-(2,2-difluoroethoxy)-N-(4-(2,5- difluorophenyl)-6-(5,5- difluorotetrahydro-2H-pyran-2- yl)pyrimidin-5-yl)isoxazole-5- carboxamide P1 |
| 469 | 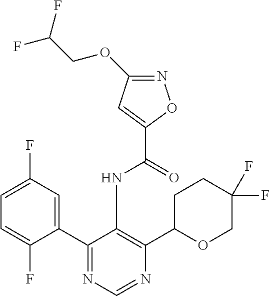 [SMILES: O=C(Nc1c(-c2cc(F)ccc2F)ncnc1C1CCC(F)(F)CO1)c1cc(OCC(F)F)no1] | 3-(2,2-difluoroethoxy)-N-(4-(2,5- difluorophenyl)-6-(5,5- difluorotetrahydro-2H-pyran-2- yl)pyrimidin-5-yl)isoxazole-5- carboxamide P2 |
| 470 | 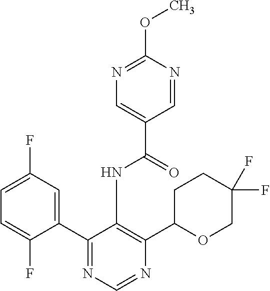 [SMILES: COc1ncc(C(=O)Nc2c(-c3cc(F)ccc3F)ncnc2C2CCC(F)(F)CO2)cn1] | N-(4-(2,5-difluorophenyl)-6-(5,5- difluorotetrahydro-2H-pyran-2- yl)pyrimidin-5-yl)-2- methoxypyrimidine-5- carboxamide P1 |
| 471 | 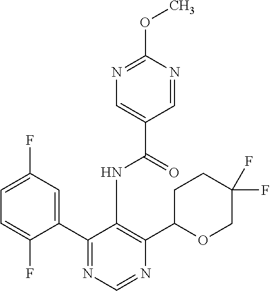 [SMILES: COc1ncc(C(=O)Nc2c(-c3cc(F)ccc3F)ncnc2C2CCC(F)(F)CO2)cn1] | -(4-(2,5-difluorophenyl)-6-(5,5- difluorotetrahydro-2H-pyran-2- yl)pyrimidin-5-yl)-2- methoxypyrimidine-5- carboxamide P2 |
| 472 | 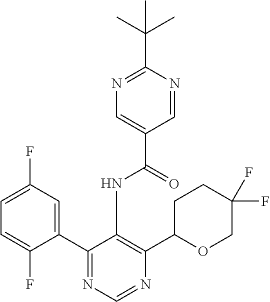 [SMILES: CC(C)(C)c1ncc(C(=O)Nc2c(-c3cc(F)ccc3F)ncnc2C2CCC(F)(F)CO2)cn1] | -(tert-butyl)-N-(4-(2,5- difluorophenyl)-6-(5,5- difluorotetrahydro-2H-pyran-2- yl)pyrimidin-5-yl)pyrimidine-5- carboxamide P1 |
| 473 | 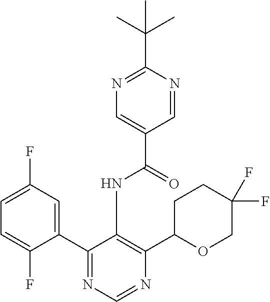 [SMILES: CC(C)(C)c1ncc(C(=O)Nc2c(-c3cc(F)ccc3F)ncnc2C2CCC(F)(F)CO2)cn1] | 2-(tert-butyl)-N-(4-(2,5- difluorophenyl)-6-(5,5- difluorotetrahydro-2H-pyran-2- yl)pyrimidin-5-yl)pyrimidine-5- carboxamide P2 |
| 474 | 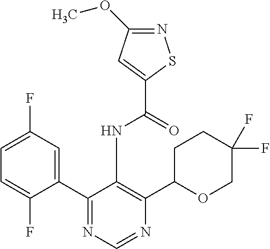 [SMILES: COc1cc(C(=O)Nc2c(-c3cc(F)ccc3F)ncnc2C2CCC(F)(F)CO2)sn1] | N-(4-(2,5-difluorophenyl)-6-(5,5- difluorotetrahydro-2H-pyran-2- yl)pyrimidin-5-yl)-3- methoxyisothiazole-5- carboxamide P1 |
| 475 | 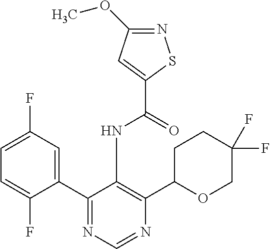 [SMILES: COc1cc(C(=O)Nc2c(-c3cc(F)ccc3F)ncnc2C2CCC(F)(F)CO2)sn1] | N-(4-(2,5-difluorophenyl)-6-(5,5- difluorotetrahydro-2H-pyran-2- yl)pyrimidin-5-yl)-3- methoxyisothiazole-5- carboxamide P2 |
| 476 | 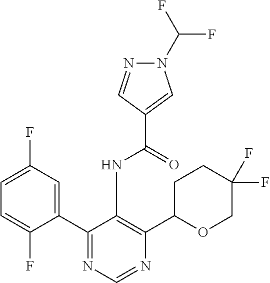 [SMILES: O=C(Nc1c(-c2cc(F)ccc2F)ncnc1C1CCC(F)(F)CO1)c1cnn(C(F)F)c1] | 1-(difluoromethyl)-N-(4-(2,5- difluorophenyl)-6-(5,5- difluorotetrahydro-2H-pyran-2- yl)pyrimidin-5-yl)-1H-pyrazole-4- carboxamide P1 |
| 477 | 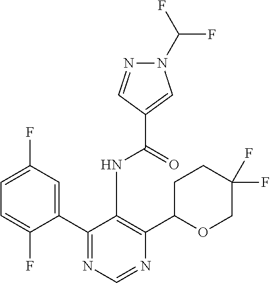 [SMILES: O=C(Nc1c(-c2cc(F)ccc2F)ncnc1C1CCC(F)(F)CO1)c1cnn(C(F)F)c1] | 1-(difluoromethyl)-N-(4-(2,5- difluorophenyl)-6-(5,5- difluorotetrahydro-2H-pyran-2- yl)pyrimidin-5-yl)-1H-pyrazole-4- carboxamide P2 |
| 478 | 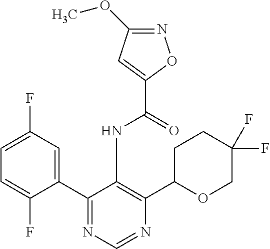 [SMILES: COc1cc(C(=O)Nc2c(-c3cc(F)ccc3F)ncnc2C2CCC(F)(F)CO2)on1] | N-(4-(2,5-difluorophenyl)-6-(5,5- difluorotetrahydro-2H-pyran-2- yl)pyrimidin-5-yl)-3- methoxyisoxazole-5- carboxamide P1 |
| 479 | 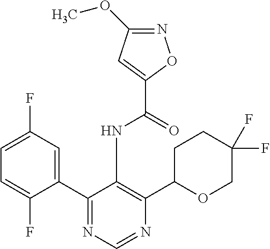 [SMILES: COc1cc(C(=O)Nc2c(-c3cc(F)ccc3F)ncnc2C2CCC(F)(F)CO2)on1] | N-(4-(2,5-difluorophenyl)-6-(5,5- difluorotetrahydro-2H-pyran-2- yl)pyrimidin-5-yl)-3- methoxyisoxazole-5- carboxamide P2 |
| 480 | 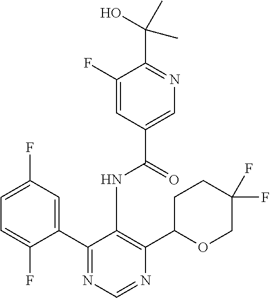 [SMILES: CC(C)(O)c1ncc(C(=O)Nc2c(-c3cc(F)ccc3F)ncnc2C2CCC(F)(F)CO2)cc1F] | N-(4-(2,5-difluorophenyl)-6-(5,5- difluorotetrahydro-2H-pyran-2- yl)pyrimidin-5-yl)-5-fluoro-6-(2- hydroxypropan-2-yl)nicotinamide P1 |
| 481 | 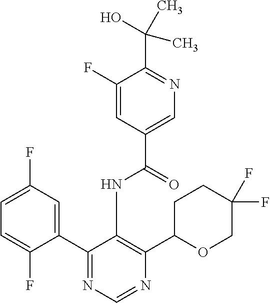 [SMILES: CC(C)(O)c1ncc(C(=O)Nc2c(-c3cc(F)ccc3F)ncnc2C2CCC(F)(F)CO2)cc1F] | N-(4-(2,5-difluorophenyl)-6-(5,5- difluorotetrahydro-2H-pyran-2- yl)pyrimidin-5-yl)-5-fluoro-6-(2- hydroxypropan-2-yl)nicotinamide P2 |
| 482 | 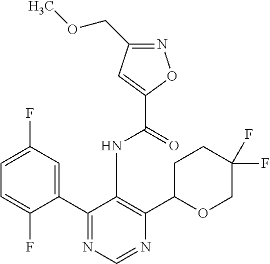 [SMILES: COCc1cc(C(=O)Nc2c(-c3cc(F)ccc3F)ncnc2C2CCC(F)(F)CO2)on1] | N-(4-(2,5-difluorophenyl)-6-(5,5- difluorotetrahydro-2H-pyran-2- yl)pyrimidin-5-yl)-3- (methoxymethyl)isoxazole-5- carboxamide P1 |
| 483 | 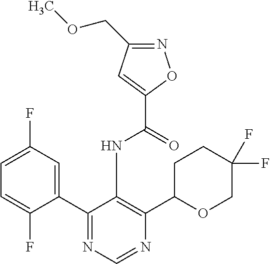 [SMILES: COCc1cc(C(=O)Nc2c(-c3cc(F)ccc3F)ncnc2C2CCC(F)(F)CO2)on1] | N-(4-(2,5-difluorophenyl)-6-(5,5- difluorotetrahydro-2H-pyran-2- yl)pyrimidin-5-yl)-3- (methoxymethyl)isoxazole-5- carboxamide P2 |
| 484 | 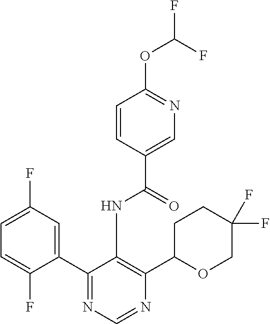 [SMILES: O=C(Nc1c(-c2cc(F)ccc2F)ncnc1C1CCC(F)(F)CO1)c1ccc(OC(F)F)nc1] | 6-(difluoromethoxy)-N-(4-(2,5- difluorophenyl)-6-(5,5- difluorotetrahydro-2H-pyran-2- yl)pyrimidin-5-yl)nicotinamide P1 |
| 485 | 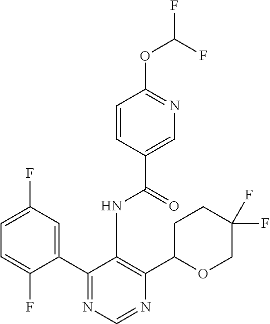 [SMILES: O=C(Nc1c(-c2cc(F)ccc2F)ncnc1C1CCC(F)(F)CO1)c1ccc(OC(F)F)nc1] | 6-(difluoromethoxy)-N-(4-(2,5- difluorophenyl)-6-(5,5- difluorotetrahydro-2H-pyran-2- yl)pyrimidin-5-yl)nicotinamide P2 |
| 486 | 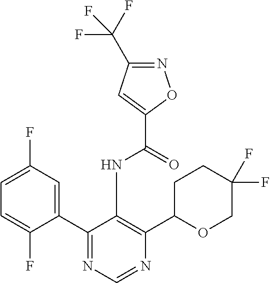 [SMILES: O=C(Nc1c(-c2cc(F)ccc2F)ncnc1C1CCC(F)(F)CO1)c1cc(C(F)(F)F)no1] | N-(4-(2,5-difluorophenyl)-6-(5,5- difluorotetrahydro-2H-pyran-2- yl)pyrimidin-5-yl)-3- (trifluoromethyl)isoxazole-5- carboxamide P1 |
| 487 | 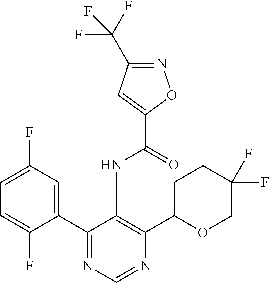 [SMILES: O=C(Nc1c(-c2cc(F)ccc2F)ncnc1C1CCC(F)(F)CO1)c1cc(C(F)(F)F)no1] | N-(4-(2,5-difluorophenyl)-6-(5,5- difluorotetrahydro-2H-pyran-2- yl)pyrimidin-5-yl)-3- (trifluoromethyl)isoxazole-5- carboxamide P2 |
| 488 | 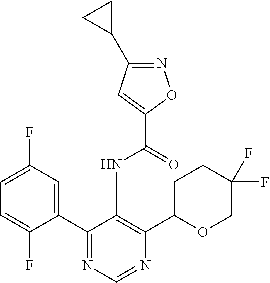 [SMILES: O=C(Nc1c(-c2cc(F)ccc2F)ncnc1C1CCC(F)(F)CO1)c1cc(C2CC2)no1] | 3-cyclopropyl-N-(4-(2,5- difluorophenyl)-6-(5,5- difluorotetrahydro-2H-pyran-2- yl)pyrimidin-5-yl)isoxazole-5- carboxamide P1 |
| 489 | 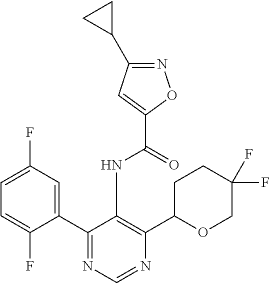 [SMILES: O=C(Nc1c(-c2cc(F)ccc2F)ncnc1C1CCC(F)(F)CO1)c1cc(C2CC2)no1] | 3-cyclopropyl-N-(4-(2,5- difluorophenyl)-6-(5,5- difluorotetrahydro-2H-pyran-2- yl)pyrimidin-5-yl)isoxazole-5- carboxamide P2 |
| 490 | 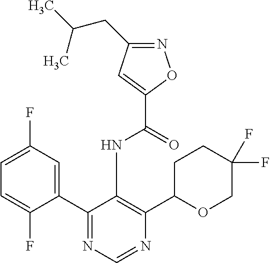 [SMILES: CC(C)Cc1cc(C(=O)Nc2c(-c3cc(F)ccc3F)ncnc2C2CCC(F)(F)CO2)on1] | N-(4-(2,5-difluorophenyl)-6-(5,5- difluorotetrahydro-2H-pyran-2- yl)pyrimidin-5-yl)-3- isobutylisoxazole-5-carboxamide P1 |
| 491 | 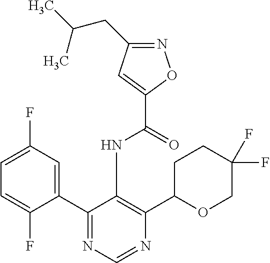 [SMILES: CC(C)Cc1cc(C(=O)Nc2c(-c3cc(F)ccc3F)ncnc2C2CCC(F)(F)CO2)on1] | N-(4-(2,5-difluorophenyl)-6-(5,5- difluorotetrahydro-2H-pyran-2- yl)pyrimidin-5-yl)-3- isobutylisoxazole-5-carboxamide P2 |
| 492 | 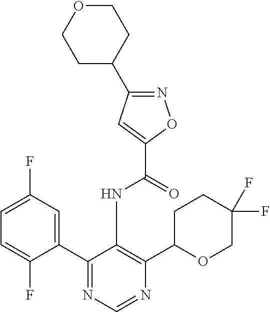 [SMILES: O=C(Nc1c(-c2cc(F)ccc2F)ncnc1C1CCC(F)(F)CO1)c1cc(C2CCOCC2)no1] | N-(4-(2,5-difluorophenyl)-6-(5,5- difluorotetrahydro-2H-pyran-2- yl)pyrimidin-5-yl)-3-(tetrahydro- 2H-pyran-4-yl)isoxazole-5- carboxamide P1 |
| 493 | 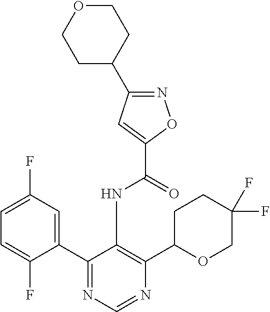 [SMILES: O=C(Nc1c(-c2cc(F)ccc2F)ncnc1C1CCC(F)(F)CO1)c1cc(C2CCOCC2)no1] | N-(4-(2,5-difluorophenyl)-6-(5,5- difluorotetrahydro-2H-pyran-2- yl)pyrimidin-5-yl)-3-(tetrahydro- 2H-pyran-4-yl)isoxazole-5- carboxamide P2 |
| 494 | 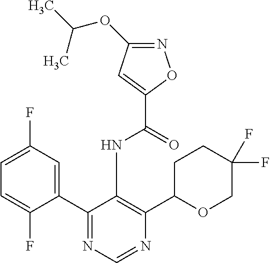 [SMILES: CC(C)Oc1cc(C(=O)Nc2c(-c3cc(F)ccc3F)ncnc2C2CCC(F)(F)CO2)on1] | N-(4-(2,5-difluorophenyl)-6-(5,5- difluorotetrahydro-2H-pyran-2- yl)pyrimidin-5-yl)-3- isopropoxyisoxazole-5- carboxamide P1 |
| 495 | 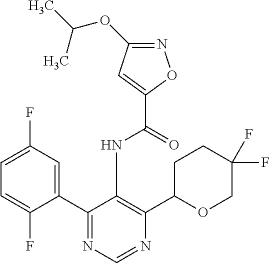 [SMILES: CC(C)Oc1cc(C(=O)Nc2c(-c3cc(F)ccc3F)ncnc2C2CCC(F)(F)CO2)on1] | N-(4-(2,5-difluorophenyl)-6-(5,5- difluorotetrahydro-2H-pyran-2- yl)pyrimidin-5-yl)-3- isopropoxyisoxazole-5- carboxamide P2 |
| 496 | 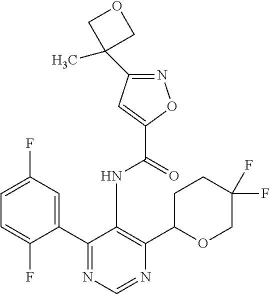 [SMILES: CC1(c2cc(C(=O)Nc3c(-c4cc(F)ccc4F)ncnc3C3CCC(F)(F)CO3)on2)COC1] | N-(4-(2,5-difluorophenyl)-6-(5,5- difluorotetrahydro-2H-pyran-2- yl)pyrimidin-5-yl)-3-(3- methyloxetan-3-yl)isoxazole-5- carboxamide P1 |
| 497 | 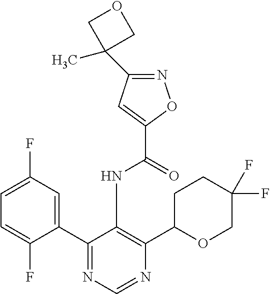 [SMILES: CC1(c2cc(C(=O)Nc3c(-c4cc(F)ccc4F)ncnc3C3CCC(F)(F)CO3)on2)COC1] | N-(4-(2,5-difluorophenyl)-6-(5,5- difluorotetrahydro-2H-pyran-2- yl)pyrimidin-5-yl)-3-(3- methyloxetan-3-yl)isoxazole-5- carboxamide P2 |
| 498 | 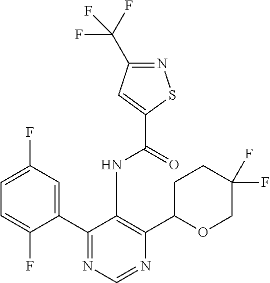 [SMILES: O=C(Nc1c(-c2cc(F)ccc2F)ncnc1C1CCC(F)(F)CO1)c1cc(C(F)(F)F)ns1] | N-(4-(2,5-difluorophenyl)-6-(5,5- difluorotetrahydro-2H-pyran-2- yl)pyrimidin-5-yl)-3- (trifluoromethyl)isothiazole-5- carboxamide P1 |
| 499 | 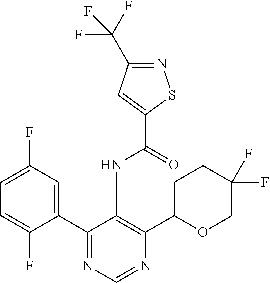 [SMILES: O=C(Nc1c(-c2cc(F)ccc2F)ncnc1C1CCC(F)(F)CO1)c1cc(C(F)(F)F)ns1] | N-(4-(2,5-difluorophenyl)-6-(5,5- difluorotetrahydro-2H-pyran-2- yl)pyrimidin-5-yl)-3- (trifluoromethyl)isothiazole-5- carboxamide P2 |
| 500 | 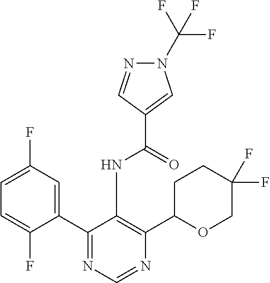 [SMILES: O=C(Nc1c(-c2cc(F)ccc2F)ncnc1C1CCC(F)(F)CO1)c1cnn(C(F)(F)F)c1] | N-(4-(2,5-difluorophenyl)-6-(5,5- difluorotetrahydro-2H-pyran-2- yl)pyrimidin-5-yl)-1- (trifluoromethyl)-1H-pyrazole-4- carboxamide P1 |
| 501 | 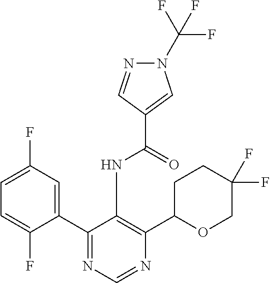 [SMILES: O=C(Nc1c(-c2cc(F)ccc2F)ncnc1C1CCC(F)(F)CO1)c1cnn(C(F)(F)F)c1] | N-(4-(2,5-difluorophenyl)-6-(5,5- difluorotetrahydro-2H-pyran-2- yl)pyrimidin-5-yl)-1- (trifluoromethyl)-1H-pyrazole-4- carboxamide P2 |
| 502 | 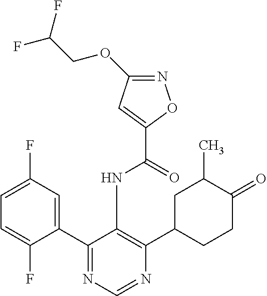 [SMILES: CC1CC(c2ncnc(-c3cc(F)ccc3F)c2NC(=O)c2cc(OCC(F)F)no2)CCC1=O] | 3-(2,2-difluoroethoxy)-N-(4-(2,5- difluorophenyl)-6-(3-methyl-4- oxocyclohexyl)pyrimidin-5- yl)isoxazole-5-carboxamide |
| 503 | 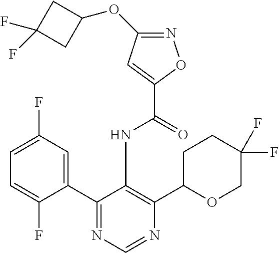 [SMILES: O=C(Nc1c(-c2cc(F)ccc2F)ncnc1C1CCC(F)(F)CO1)c1cc(OC2CC(F)(F)C2)no1] | 3-(3,3-difluorocyclobutoxy)-N-(4- (2,5-difluorophenyl)-6-(5,5- difluorotetrahydro-2H-pyran-2- yl)pyrimidin-5-yl)isoxazole-5- carboxamide P1 |
| 504 | 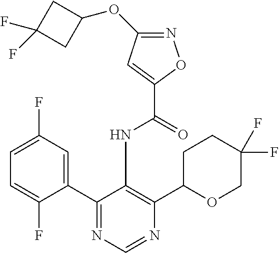 [SMILES: O=C(Nc1c(-c2cc(F)ccc2F)ncnc1C1CCC(F)(F)CO1)c1cc(OC2CC(F)(F)C2)no1] | 3-(3,3-difluorocyclobutoxy)-N-(4- (2,5-difluorophenyl)-6-(5,5- difluorotetrahydro-2H-pyran-2- yl)pyrimidin-5-yl)isoxazole-5- carboxamide P2 |
| 505 | 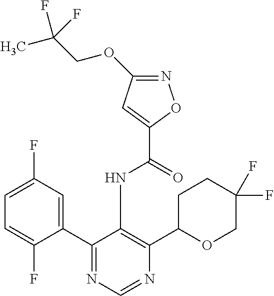 [SMILES: CC(F)(F)COc1cc(C(=O)Nc2c(-c3cc(F)ccc3F)ncnc2C2CCC(F)(F)CO2)on1] | N-(4-(2,5-difluorophenyl)-6-(5,5- difluorotetrahydro-2H-pyran-2- yl)pyrimidin-5-yl)-3-(2,2- difluoropropoxy)isoxazole-5- carboxamide |
| 506 | 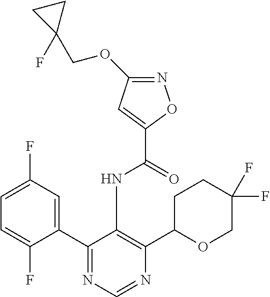 [SMILES: O=C(Nc1c(-c2cc(F)ccc2F)ncnc1C1CCC(F)(F)CO1)c1cc(OCC2(F)CC2)no1] | N-(4-(2,5-difluorophenyl)-6-(5,5- difluorotetrahydro-2H-pyran-2- yl)pyrimidin-5-yl)-3-((1- fluorocyclopropyl)methoxy) isoxazole-5-carboxamide |
| 507 | 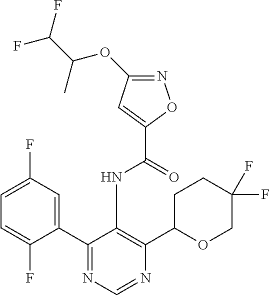 [SMILES: CC(Oc1cc(C(=O)Nc2c(-c3cc(F)ccc3F)ncnc2C2CCC(F)(F)CO2)on1)C(F)F] | N-(4-(2,5-difluorophenyl)-6-(5,5- difluorotetrahydro-2H-pyran-2- yl)pyrimidin-5-yl)-3-((1,1- difluoropropan-2- yl)oxy)isoxazole-5-carboxamide |
| 508 | 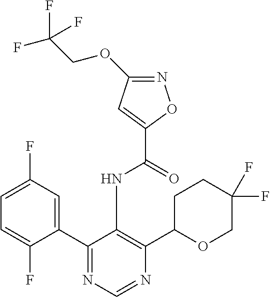 [SMILES: O=C(Nc1c(-c2cc(F)ccc2F)ncnc1C1CCC(F)(F)CO1)c1cc(OCC(F)(F)F)no1] | N-(4-(2,5-difluorophenyl)-6-(5,5- difluorotetrahydro-2H-pyran-2- yl)pyrimidin-5-yl)-3-(2,2,2- trifluoroethoxy)isoxazole-5- carboxamide |
| 509 | 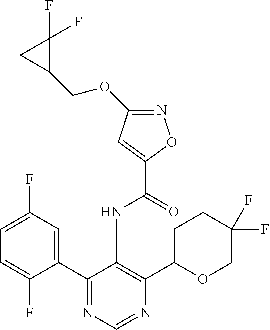 [SMILES: O=C(Nc1c(-c2cc(F)ccc2F)ncnc1C1CCC(F)(F)CO1)c1cc(OCC2CC2(F)F)no1] | 3-((2,2- difluorocyclopropyl)methoxy)-N- (4-(2,5-difluorophenyl)-6-(5,5- difluorotetrahydro-2H-pyran-2- yl)pyrimidin-5-yl)isoxazole-5- carboxamide |
| 510 | 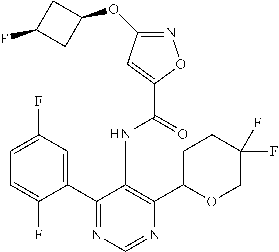 [SMILES: O=C(Nc1c(-c2cc(F)ccc2F)ncnc1C1CCC(F)(F)CO1)c1cc(O[C@H]2C[C@@H](F)C2)no1] | N-(4-(2,5-difluorophenyl)-6-(5,5- difluorotetrahydro-2H-pyran-2- yl)pyrimidin-5-yl)-syn-3- fluorocyclobutoxy)isoxazole-5- carboxamide |
| 511 | 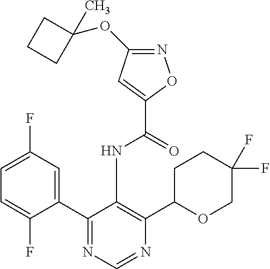 [SMILES: CC1(Oc2cc(C(=O)Nc3c(-c4cc(F)ccc4F)ncnc3C3CCC(F)(F)CO3)on2)CCC1] | N-(4-(2,5-difluorophenyl)-6-(5,5- difluorotetrahydro-2H-pyran-2- yl)pyrimidin-5-yl)-3-(1- methylcyclobutoxy)isoxazole-5- carboxamide |
| 512 | 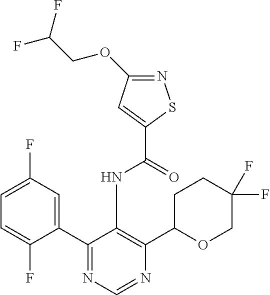 [SMILES: O=C(Nc1c(-c2cc(F)ccc2F)ncnc1C1CCC(F)(F)CO1)c1cc(OCC(F)F)ns1] | 3-(2,2-difluoroethoxy)-N-(4-(2,5- difluorophenyl)-6-(5,5- difluorotetrahydro-2H-pyran-2- yl)pyrimidin-5-yl)isothiazole-5- carboxamide |
| 513 | 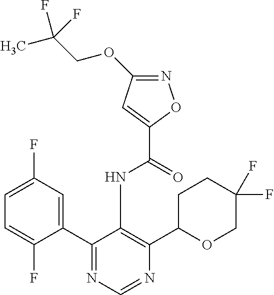 [SMILES: CC(F)(F)COc1cc(C(=O)Nc2c(-c3cc(F)ccc3F)ncnc2C2CCC(F)(F)CO2)on1] | N-(4-(2,5-difluorophenyl)-6-(5,5- difluorotetrahydro-2H-pyran-2- yl)pyrimidin-5-yl)-3-((1- fluorocyclopropyl)methoxy) isoxazole-5-carboxamide P1 |
| 514 | 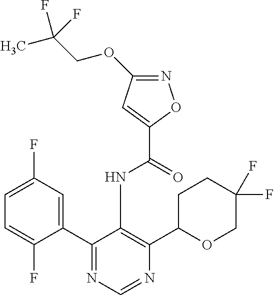 [SMILES: CC(F)(F)COc1cc(C(=O)Nc2c(-c3cc(F)ccc3F)ncnc2C2CCC(F)(F)CO2)on1] | N-(4-(2,5-difluorophenyl)-6-(5,5- difluorotetrahydro-2H-pyran-2- yl)pyrimidin-5-yl)-3-((1- fluorocyclopropyl)methoxy) isoxazole-5-carboxamide P2 |
| 515 | 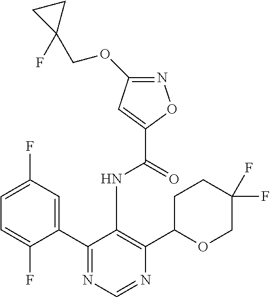 [SMILES: O=C(Nc1c(-c2cc(F)ccc2F)ncnc1C1CCC(F)(F)CO1)c1cc(OCC2(F)CC2)no1] | N-(4-(2,5-difluorophenyl)-6-(5,5- difluorotetrahydro-2H-pyran-2- yl)pyrimidin-5-yl)-3-(2,2- difluoropropoxy)isoxazole-5- carboxamide P1 |
| 516 | 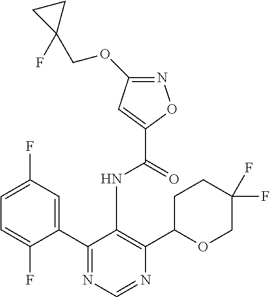 [SMILES: O=C(Nc1c(-c2cc(F)ccc2F)ncnc1C1CCC(F)(F)CO1)c1cc(OCC2(F)CC2)no1] | N-(4-(2,5-difluorophenyl)-6-(5,5- difluorotetrahydro-2H-pyran-2- yl)pyrimidin-5-yl)-3-(2,2- difluoropropoxy)isoxazole-5- carboxamide P2 |
| 517 | 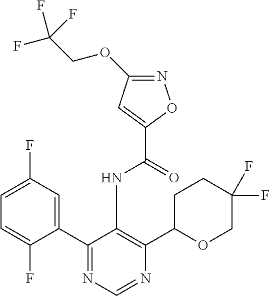 [SMILES: O=C(Nc1c(-c2cc(F)ccc2F)ncnc1C1CCC(F)(F)CO1)c1cc(OCC(F)(F)F)no1] | N-(4-(2,5-difluorophenyl)-6-(5,5- difluorotetrahydro-2H-pyran-2- yl)pyrimidin-5-yl)-3-(2,2,2- trifluoroethoxy)isoxazole-5- carboxamide P1 |
| 518 | 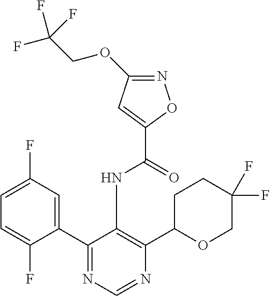 [SMILES: O=C(Nc1c(-c2cc(F)ccc2F)ncnc1C1CCC(F)(F)CO1)c1cc(OCC(F)(F)F)no1] | N-(4-(2,5-difluorophenyl)-6-(5,5- difluorotetrahydro-2H-pyran-2- yl)pyrimidin-5-yl)-3-(2,2,2- trifluoroethoxy)isoxazole-5- carboxamide P2 |
| 519 | 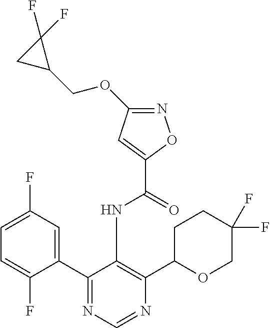 [SMILES: O=C(Nc1c(-c2cc(F)ccc2F)ncnc1C1CCC(F)(F)CO1)c1cc(OCC2CC2(F)F)no1] | 3-((2,2- difluorocyclopropyl)methoxy)-N- (4-(2,5-difluorophenyl)-6-(5,5- difluorotetrahydro-2H-pyran-2- yl)pyrimidin-5-yl)isoxazole-5- carboxamide P1 |
| 520 | 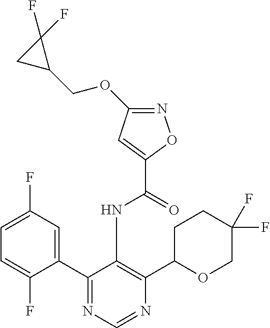 [SMILES: O=C(Nc1c(-c2cc(F)ccc2F)ncnc1C1CCC(F)(F)CO1)c1cc(OCC2CC2(F)F)no1] | 3-((2,2- difluorocyclopropyl)methoxy)-N- (4-(2,5-difluorophenyl)-6-(5,5- difluorotetrahydro-2H-pyran-2- yl)pyrimidin-5-yl)isoxazole-5- carboxamide P2 |
| 521 | 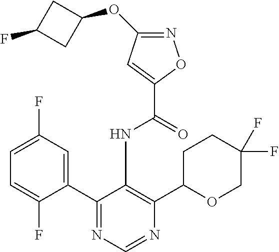 [SMILES: O=C(Nc1c(-c2cc(F)ccc2F)ncnc1C1CCC(F)(F)CO1)c1cc(O[C@H]2C[C@@H](F)C2)no1] | N-(4-(2,5-difluorophenyl)-6-(5,5- difluorotetrahydro-2H-pyran-2- yl)pyrimidin-5-yl)-3-(syn-3- fluorocyclobutoxy)isoxazole-5- carboxamide P1 |
| 522 | 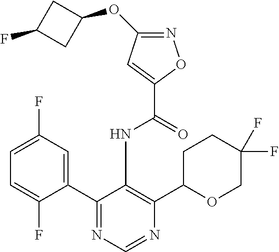 [SMILES: O=C(Nc1c(-c2cc(F)ccc2F)ncnc1C1CCC(F)(F)CO1)c1cc(O[C@H]2C[C@@H](F)C2)no1] | N-(4-(2,5-difluorophenyl)-6-(5,5- difluorotetrahydro-2H-pyran-2- yl)pyrimidin-5-yl)-3-(syn-3- fluorocyclobutoxy)isoxazole-5- carboxamide P2 |
| 523 | 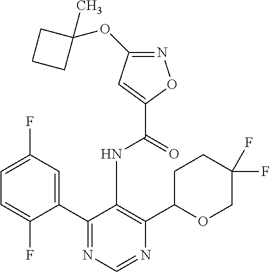 [SMILES: CC1(Oc2cc(C(=O)Nc3c(-c4cc(F)ccc4F)ncnc3C3CCC(F)(F)CO3)on2)CCC1] | N-(4-(2,5-difluorophenyl)-6-(5,5- difluorotetrahydro-2H-pyran-2- yl)pyrimidin-5-yl)-3-(1- methylcyclobutoxy)isoxazole-5- carboxamide P1 |
| 524 | 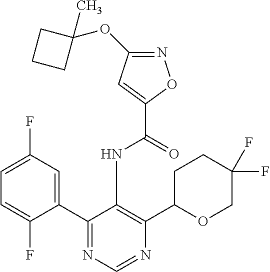 [SMILES: CC1(Oc2cc(C(=O)Nc3c(-c4cc(F)ccc4F)ncnc3C3CCC(F)(F)CO3)on2)CCC1] | N-(4-(2,5-difluorophenyl)-6-(5,5- difluorotetrahydro-2H-pyran-2- yl)pyrimidin-5-yl)-3-(1- methylcyclobutoxy)isoxazole-5- carboxamide P2 |
| 525 | 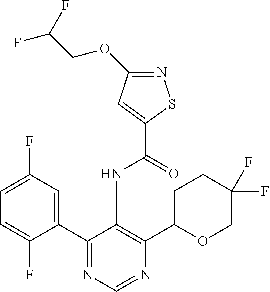 [SMILES: O=C(Nc1c(-c2cc(F)ccc2F)ncnc1C1CCC(F)(F)CO1)c1cc(OCC(F)F)ns1] | 3-(2,2-difluoroethoxy)-N-(4-(2,5- difluorophenyl)-6-(5,5- difluorotetrahydro-2H-pyran-2- yl)pyrimidin-5-yl)isothiazole-5- carboxamide P1 |
| 526 | 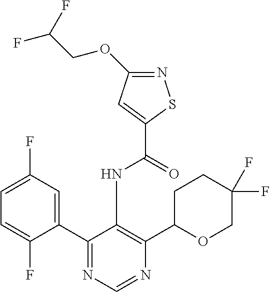 [SMILES: O=C(Nc1c(-c2cc(F)ccc2F)ncnc1C1CCC(F)(F)CO1)c1cc(OCC(F)F)ns1] | 3-(2,2-difluoroethoxy)-N-(4-(2,5- difluorophenyl)-6-(5,5- difluorotetrahydro-2H-pyran-2- yl)pyrimidin-5-yl)isothiazole-5- carboxamide P2 |
| 527 | 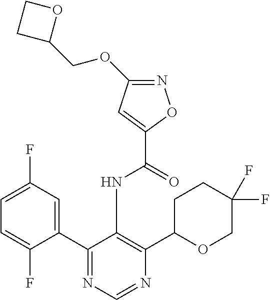 [SMILES: O=C(Nc1c(-c2cc(F)ccc2F)ncnc1C1CCC(F)(F)CO1)c1cc(OCC2CCO2)no1] | N-(4-(2,5-difluorophenyl)-6-(5,5- difluorotetrahydro-2H-pyran-2- yl)pyrimidin-5-yl)-3-(oxetan-2- ylmethoxy)isoxazole-5- carboxamide |
| 528 | 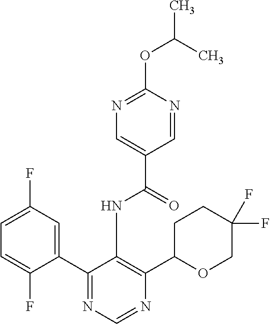 [SMILES: CC(C)Oc1ncc(C(=O)Nc2c(-c3cc(F)ccc3F)ncnc2C2CCC(F)(F)CO2)cn1] | N-(4-(2,5-difluorophenyl)-6-(5,5- difluorotetrahydro-2H-pyran-2- yl)pyrimidin-5-yl)-2- isopropoxypyrimidine-5- carboxamide P1 |
| 529 | 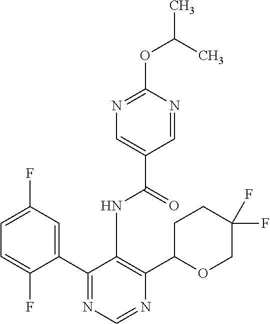 [SMILES: CC(C)Oc1ncc(C(=O)Nc2c(-c3cc(F)ccc3F)ncnc2C2CCC(F)(F)CO2)cn1] | N-(4-(2,5-difluorophenyl)-6-(5,5- difluorotetrahydro-2H-pyran-2- yl)pyrimidin-5-yl)-2- isopropoxypyrimidine-5- carboxamide P2 |
| 530 | 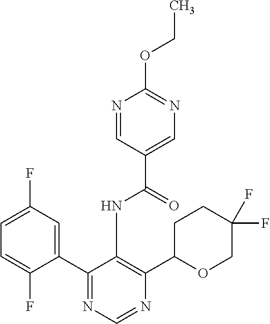 [SMILES: CCOc1ncc(C(=O)Nc2c(-c3cc(F)ccc3F)ncnc2C2CCC(F)(F)CO2)cn1] | N-(4-(2,5-difluorophenyl)-6-(5,5- difluorotetrahydro-2H-pyran-2- yl)pyrimidin-5-yl)-2- ethoxypyrimidine-5-carboxamide |
| 531 | 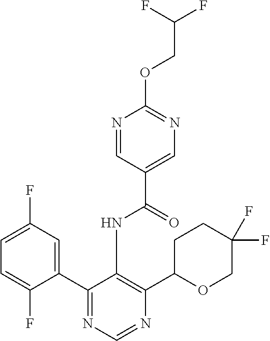 [SMILES: O=C(Nc1c(-c2cc(F)ccc2F)ncnc1C1CCC(F)(F)CO1)c1cnc(OCC(F)F)nc1] | 2-(2,2-difluoroethoxy)-N-(4-(2,5- difluorophenyl)-6-(5,5- difluorotetrahydro-2H-pyran-2- yl)pyrimidin-5-yl)pyrimidine-5- carboxamide |
| 532 | 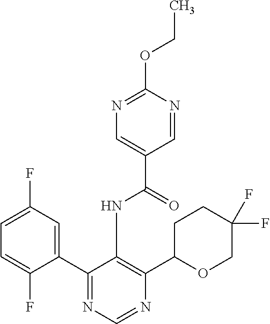 [SMILES: CCOc1ncc(C(=O)Nc2c(-c3cc(F)ccc3F)ncnc2C2CCC(F)(F)CO2)cn1] | N-(4-(2,5-difluorophenyl)-6-(5,5- difluorotetrahydro-2H-pyran-2- yl)pyrimidin-5-yl)-2- ethoxypyrimidine-5-carboxamide P1 |
| 533 | 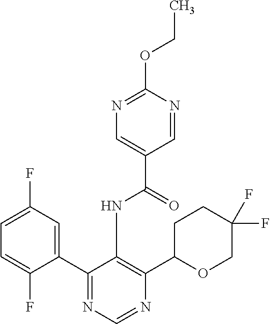 [SMILES: CCOc1ncc(C(=O)Nc2c(-c3cc(F)ccc3F)ncnc2C2CCC(F)(F)CO2)cn1] | N-(4-(2,5-difluorophenyl)-6-(5,5- difluorotetrahydro-2H-pyran-2- yl)pyrimidin-5-yl)-2- ethoxypyrimidine-5-carboxamide P2 |
| 534 | 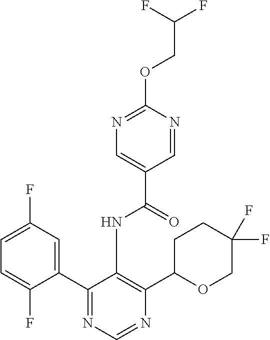 [SMILES: O=C(Nc1c(-c2cc(F)ccc2F)ncnc1C1CCC(F)(F)CO1)c1cnc(OCC(F)F)nc1] | 2-(2,2-difluoroethoxy)-N-(4-(2,5- difluorophenyl)-6-(5,5- difluorotetrahydro-2H-pyran-2- yl)pyrimidin-5-yl)pyrimidine-5- carboxamide P1 |
| 535 | 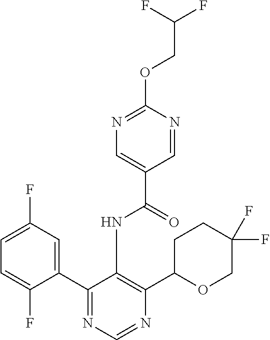 [SMILES: O=C(Nc1c(-c2cc(F)ccc2F)ncnc1C1CCC(F)(F)CO1)c1cnc(OCC(F)F)nc1] | 2-(2,2-difluoroethoxy)-N-(4-(2,5- difluorophenyl)-6-(5,5- difluorotetrahydro-2H-pyran-2- yl)pyrimidin-5-yl)pyrimidine-5- carboxamide P2 |
| 536 | 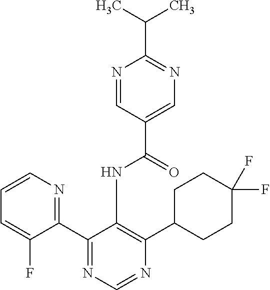 [SMILES: CC(C)c1ncc(C(=O)Nc2c(-c3ncccc3F)ncnc2C2CCC(F)(F)CC2)cn1] | N-(4-(4,4-difluorocyclohexyl)-6- (3-fluoropyridin-2-yl)pyrimidin-5- yl)-2-isopropylpyrimidine-5- carboxamide |
| 537 | 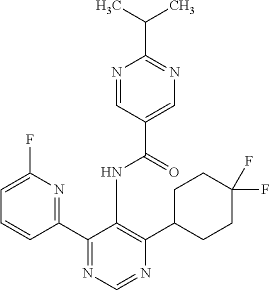 [SMILES: CC(C)c1ncc(C(=O)Nc2c(-c3cccc(F)n3)ncnc2C2CCC(F)(F)CC2)cn1] | N-(4-(4,4-difluorocyclohexyl)-6- (6-fluoropyridin-2-yl)pyrimidin-5- yl)-2-isopropylpyrimidine-5- carboxamide |
| 538 | 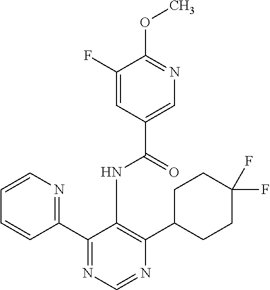 [SMILES: COc1ncc(C(=O)Nc2c(-c3ccccn3)ncnc2C2CCC(F)(F)CC2)cc1F] | N-(4-(4,4-difluorocyclohexyl)-6- (pyridin-2-yl)pyrimidin-5-yl)-5- fluoro-6-methoxynicotinamide |
| 539 | 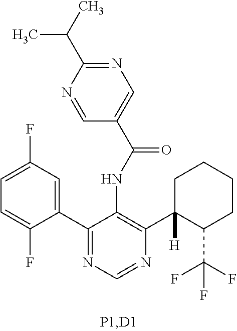 [SMILES: CC(C)c1ncc(C(=O)Nc2c(-c3cc(F)ccc3F)ncnc2[C@@H]2CCCC[C@@H]2C(F)(F)F)cn1] | N-(4-(2,5-difluorophenyl)-6-(syn- 2-(trifluoromethyl)cyclohexyl) pyrimidin-5-yl)-2- isopropylpyrimidine-5- carboxamide P1,D1 |
| 540 | 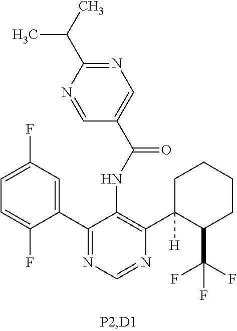 [SMILES: CC(C)c1ncc(C(=O)Nc2c(-c3cc(F)ccc3F)ncnc2[C@H]2CCCC[C@H]2C(F)(F)F)cn1] | N-(4-(2,5-difluorophenyl)-6-(syn- 2-(trifluoromethyl)cyclohexyl) pyrimidin-5-yl)-2- isopropylpyrimidine-5- carboxamide P2,D1 |
| 541 | 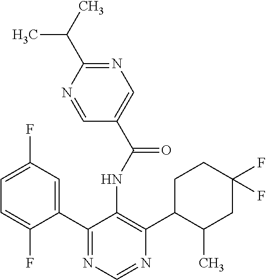 [SMILES: CC(C)c1ncc(C(=O)Nc2c(-c3cc(F)ccc3F)ncnc2C2CCC(F)(F)CC2C)cn1] | N-(4-(4,4-difluoro-2- methylcyclohexyl)-6-(2,5- difluorophenyl)pyrimidin-5-yl)-2- isopropylpyrimidine-5- carboxamide P1 |
| 542 | 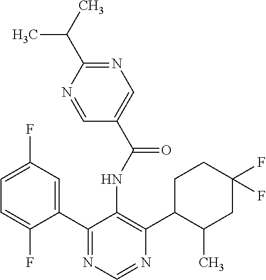 [SMILES: CC(C)c1ncc(C(=O)Nc2c(-c3cc(F)ccc3F)ncnc2C2CCC(F)(F)CC2C)cn1] | N-(4-(4,4-difluoro-2- methylcyclohexyl)-6-(2,5- difluorophenyl)pyrimidin-5-yl)-2- isopropylpyrimidine-5- carboxamide P2 |
| 543 | 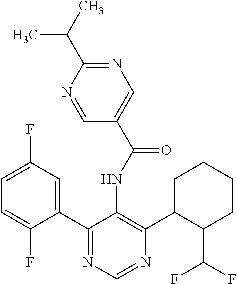 [SMILES: CC(C)c1ncc(C(=O)Nc2c(-c3cc(F)ccc3F)ncnc2C2CCCCC2C(F)F)cn1] | N-(4-(2- (difluoromethyl)cyclohexyl)-6- (2,5-difluorophenyl)pyrimidin-5- yl)-2-isopropylpyrimidine-5- carboxamide |
| 544 | 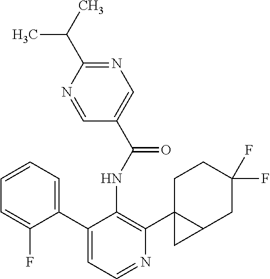 [SMILES: CC(C)c1ncc(C(=O)Nc2c(-c3ccccc3F)ccnc2C23CCC(F)(F)CC2C3)cn1] | N-(2-(4,4- difluorobicyclo[4.1.0]heptan-1- yl)-4-(2-fluorophenyl)pyridin-3- yl)-2-isopropylpyrimidine-5- carboxamide |
| 545 | 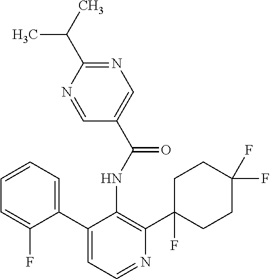 [SMILES: CC(C)c1ncc(C(=O)Nc2c(-c3ccccc3F)ccnc2C2(F)CCC(F)(F)CC2)cn1] | N-(4-(2-fluorophenyl)-2-(1,4,4- trifluorocyclohexyl)pyridin-3-yl)- 2-isopropylpyrimidine-5- carboxamide |
| 546 | 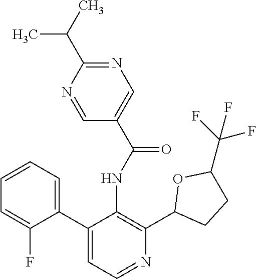 [SMILES: CC(C)c1ncc(C(=O)Nc2c(-c3ccccc3F)ccnc2C2CCC(C(F)(F)F)O2)cn1] | N-(4-(2-fluorophenyl)-2-(5- (trifluoromethyl)tetrahydrofuran- 2-yl)pyridin-3-yl)-2- isopropylpyrimidine-5- carboxamide D1 |
| 547 | 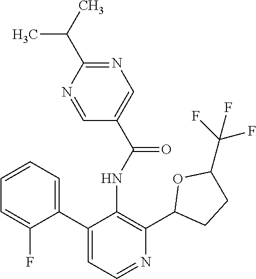 [SMILES: CC(C)c1ncc(C(=O)Nc2c(-c3ccccc3F)ccnc2C2CCC(C(F)(F)F)O2)cn1] | N-(4-(2-fluorophenyl)-2-(5- (trifluoromethyl)tetrahydrofuran- 2-yl)pyridin-3-yl)-2- isopropylpyrimidine-5- carboxamide D2 |
| 548 | 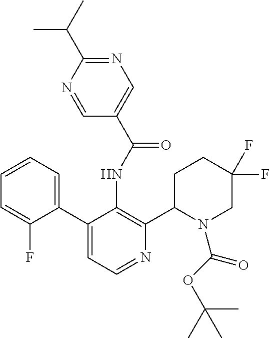 [SMILES: CC(C)c1ncc(C(=O)Nc2c(-c3ccccc3F)ccnc2C2CCC(F)(F)CN2C(=O)OC(C)(C)C)cn1] | tert-butyl 5,5-difluoro-2-(4-(2- fluorophenyl)-3-(2- isopropylpyrimidine-5- carboxamido)pyridin-2- yl)piperidine-1-carboxylate |
| 549 | 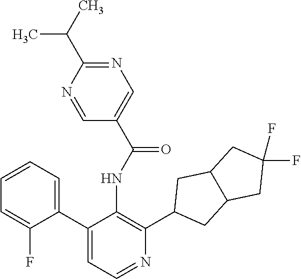 [SMILES: CC(C)c1ncc(C(=O)Nc2c(-c3ccccc3F)ccnc2C2CC3CC(F)(F)CC3C2)cn1] | N-(2-(5,5- difluorooctahydropentalen-2-yl)- 4-(2-fluorophenyl)pyridin-3-yl)-2- isopropylpyrimidine-5- carboxamide dr = 1:2 |
| 550 | 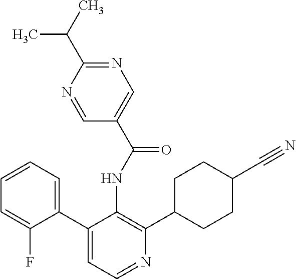 [SMILES: CC(C)c1ncc(C(=O)Nc2c(-c3ccccc3F)ccnc2C2CCC(C#N)CC2)cn1] | N-(2-(4-cyanocyclohexyl)-4-(2- fluorophenyl)pyridin-3-yl)-2- isopropylpyrimidine-5- carboxamide dr = 1:1 |
| 551 | 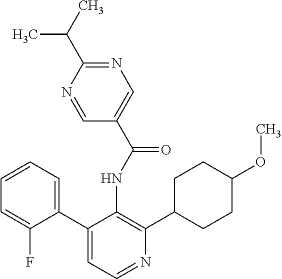 [SMILES: COC1CCC(c2nccc(-c3ccccc3F)c2NC(=O)c2cnc(C(C)C)nc2)CC1] | N-(4-(2-fluorophenyl)-2-(4- methoxycyclohexyl)pyridin-3-yl)- 2-isopropylpyrimidine-5- carboxamide D1 |
| 552 | 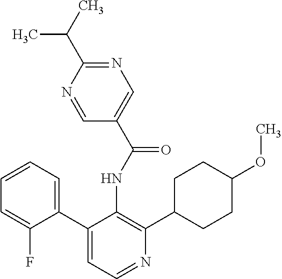 [SMILES: COC1CCC(c2nccc(-c3ccccc3F)c2NC(=O)c2cnc(C(C)C)nc2)CC1] | N-(4-(2-fluorophenyl)-2-(4- methoxycyclohexyl)pyridin-3-yl)- 2-isopropylpyrimidine-5- carboxamide D2 |
| 553 | 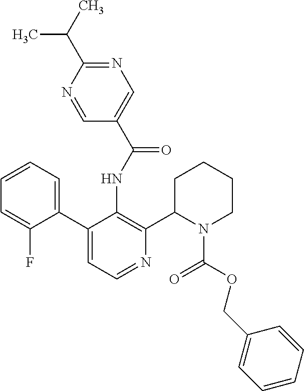 [SMILES: CC(C)c1ncc(C(=O)Nc2c(-c3ccccc3F)ccnc2C2CCCCN2C(=O)OCc2ccccc2)cn1] | Benzyl 2-(4-(2-fluorophenyl)-3- (2-isopropylpyrimidine-5- carboxamido)pyridin-2- yl)piperidine-1-carboxylate |
| 554 | 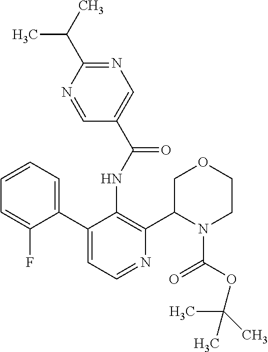 [SMILES: CC(C)c1ncc(C(=O)Nc2c(-c3ccccc3F)ccnc2C2COCCN2C(=O)OC(C)(C)C)cn1] | Tert-butyl 3-(4-(2-fluorophenyl)- 3-(2-isopropylpyrimidine-5- carboxamido)pyridin-2- yl)morpholine-4-carboxylate |
| 555 | 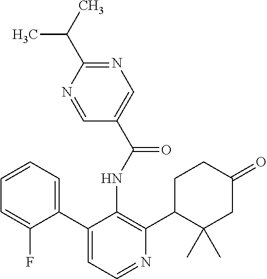 [SMILES: CC(C)c1ncc(C(=O)Nc2c(-c3ccccc3F)ccnc2C2CCC(=O)CC2(C)C)cn1] | (2-(2,2-dimethyl-4- oxocyclohexyl)-4-(2- fluorophenyl)pyridin-3-yl)-2- isopropylpyrimidine-5- carboxamide |
| 556 | 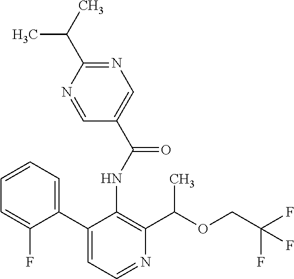 [SMILES: CC(C)c1ncc(C(=O)Nc2c(-c3ccccc3F)ccnc2C(C)OCC(F)(F)F)cn1] | N-(4-(2-fluorophenyl)-2-(1-(2,2,2- trifluoroethoxy)ethyl)pyridin-3-yl)- 2-isopropylpyrimidine-5- carboxamide |
| 557 | 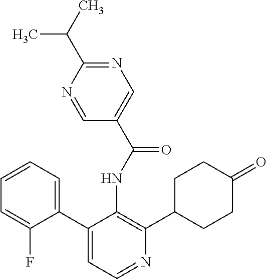 [SMILES: CC(C)c1ncc(C(=O)Nc2c(-c3ccccc3F)ccnc2C2CCC(=O)CC2)cn1] | N-(4-(2-fluorophenyl)-2-(4- oxocyclohexyl)pyridin-3-yl)-2- isopropylpyrimidine-5- carboxamide |
| 558 | 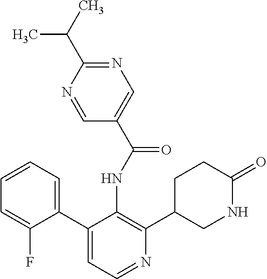 [SMILES: CC(C)c1ncc(C(=O)Nc2c(-c3ccccc3F)ccnc2C2CCC(=O)NC2)cn1] | N-(4-(2-fluorophenyl)-2-(6- oxopiperidin-3-yl)pyridin-3-yl)-2- isopropylpyrimidine-5- carboxamide |
| 559 | 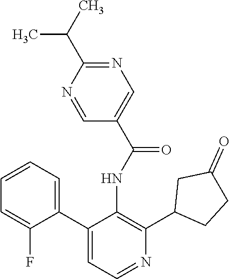 [SMILES: CC(C)c1ncc(C(=O)Nc2c(-c3ccccc3F)ccnc2C2CCC(=O)C2)cn1] | N-(4-(2-fluorophenyl)-2-(3- oxocyclopentyl)pyridin-3-yl)-2- isopropylpyrimidine-5- carboxamide |
| 560 | 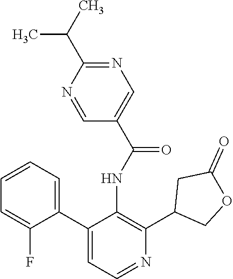 [SMILES: CC(C)c1ncc(C(=O)Nc2c(-c3ccccc3F)ccnc2C2COC(=O)C2)cn1] | N-(4-(2-fluorophenyl)-2-(5- oxotetrahydrofuran-3-yl)pyridin- 3-yl)-2-isopropylpyrimidine-5- carboxamide |
| 561 | 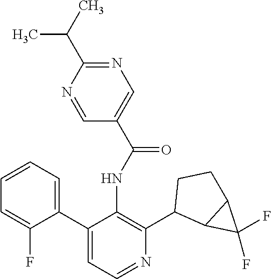 [SMILES: CC(C)c1ncc(C(=O)Nc2c(-c3ccccc3F)ccnc2C2CCC3C2C3(F)F)cn1] | N-(2-(6,6- difluorobicyclo[3.1.0]hexan-2-yl)- 4-(2-fluorophenyl)pyridin-3-yl)-2- isopropylpyrimidine-5- carboxamide |
| 562 | 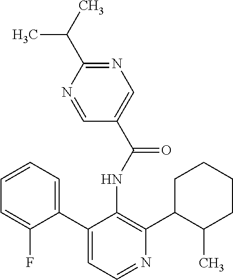 [SMILES: CC(C)c1ncc(C(=O)Nc2c(-c3ccccc3F)ccnc2C2CCCCC2C)cn1] | N-(4-(2-fluorophenyl)-2-(2- methylcyclohexyl)pyridin-3-yl)-2- isopropylpyrimidine-5- carboxamide |
| 563 | 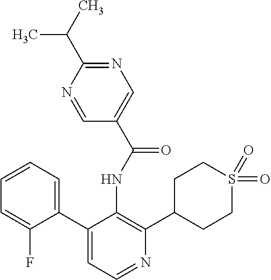 [SMILES: CC(C)c1ncc(C(=O)Nc2c(-c3ccccc3F)ccnc2C2CCS(=O)(=O)CC2)cn1] | N-(2-(1,1-dioxidotetrahydro-2H- thiopyran-4-yl)-4-(2- fluorophenyl)pyridin-3-yl)-2- isopropylpyrimidine-5- carboxamide |
| 564 | 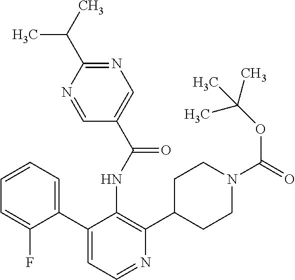 [SMILES: CC(C)c1ncc(C(=O)Nc2c(-c3ccccc3F)ccnc2C2CCN(C(=O)OC(C)(C)C)CC2)cn1] | Tert-butyl 4-(4-(2-fluorophenyl)- 3-(2-isopropylpyrimidine-5- carboxamido)pyridin-2- yl)piperidine-1-carboxylate |
| 565 | 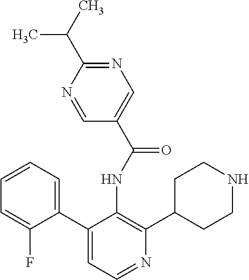 [SMILES: CC(C)c1ncc(C(=O)Nc2c(-c3ccccc3F)ccnc2C2CCNCC2)cn1] | Tert-butyl 4-(4-(2-fluorophenyl)- 3-(2-isopropylpyrimidine-5- carboxamido)pyridin-2- yl)piperidine-1-carboxylate |
| 566 | 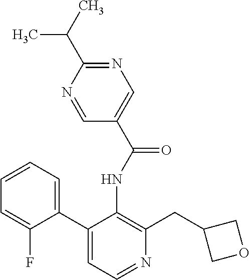 [SMILES: CC(C)c1ncc(C(=O)Nc2c(-c3ccccc3F)ccnc2CC2COC2)cn1] | N-(4-(2-fluorophenyl)-2-(oxetan- 3-ylmethyl)pyridin-3-yl)-2- isopropylpyrimidine-5- carboxamide |
| 567 | 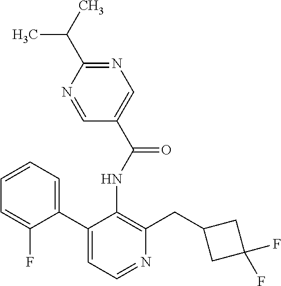 [SMILES: CC(C)c1ncc(C(=O)Nc2c(-c3ccccc3F)ccnc2CC2CC(F)(F)C2)cn1] | N-(2-((3,3- difluorocyclobutyl)methyl)-4-(2- fluorophenyl)pyridin-3-yl)-2- isopropylpyrimidine-5- carboxamide |
| 568 | 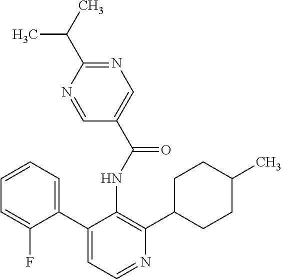 [SMILES: CC1CCC(c2nccc(-c3ccccc3F)c2NC(=O)c2cnc(C(C)C)nc2)CC1] | N-(4-(2-fluorophenyl)-2-(4- methylcyclohexyl)pyridin-3-yl)-2- isopropylpyrimidine-5- carboxamide dr = 2:1 |
| 569 | 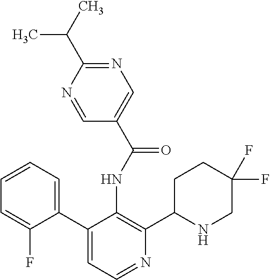 [SMILES: CC(C)c1ncc(C(=O)Nc2c(-c3ccccc3F)ccnc2C2CCC(F)(F)CN2)cn1] | N-(2-(5,5-difluoropiperidin-2-yl)- 4-(2-fluorophenyl)pyridin-3-yl)-2- isopropylpyrimidine-5- carboxamide |
| 570 | 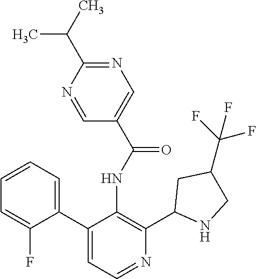 [SMILES: CC(C)c1ncc(C(=O)Nc2c(-c3ccccc3F)ccnc2C2CC(C(F)(F)F)CN2)cn1] | N-(4-(2-fluorophenyl)-2-(4- (trifluoromethyl)pyrrolidin-2- yl)pyridin-3-yl)-2- isopropylpyrimidine-5- carboxamide |
| 571 | 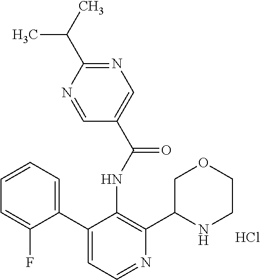 [SMILES: CC(C)c1ncc(C(=O)Nc2c(-c3ccccc3F)ccnc2C2COCCN2)cn1 ; Cl] | N-(4-(2-fluorophenyl)-2- (morpholin-3-yl)pyridin-3-yl)-2- isopropylpyrimidine-5- carboxamide hydrochloric acid salt |
| 572 | 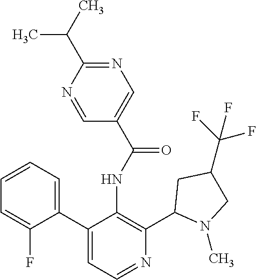 [SMILES: CC(C)c1ncc(C(=O)Nc2c(-c3ccccc3F)ccnc2C2CC(C(F)(F)F)CN2C)cn1] | N-(4-(2-fluorophenyl)-2-(1- methyl-4- (trifluoromethyl)pyrrolidin-2- yl)pyridin-3-yl)-2- isopropylpyrimidine-5- carboxamide |
| 573 | 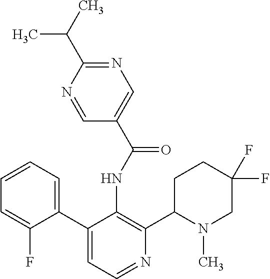 [SMILES: CC(C)c1ncc(C(=O)Nc2c(-c3ccccc3F)ccnc2C2CCC(F)(F)CN2C)cn1] | N-(2-(5,5-difluoro-1- methylpiperidin-2-yl)-4-(2- fluorophenyl)pyridin-3-yl)-2- isopropylpyrimidine-5- carboxamide |
| 574 | 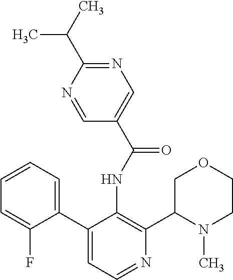 [SMILES: CC(C)c1ncc(C(=O)Nc2c(-c3ccccc3F)ccnc2C2COCCN2C)cn1] | N-(4-(2-fluorophenyl)-2-(4- methylmorpholin-3-yl)pyridin-3- yl)-2-isopropylpyrimidine-5- carboxamide |
| 575 | 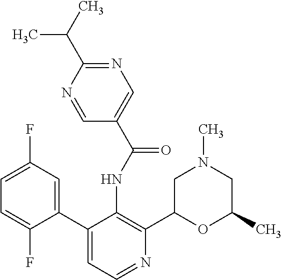 [SMILES: CC(C)c1ncc(C(=O)Nc2c(-c3cc(F)ccc3F)ccnc2C2CN(C)C[C@@H](C)O2)cn1] | N-(4-(2,5-difluorophenyl)-2- ((6R)-4,6-dimethylmorpholin-2- yl)pyridin-3-yl)-2- isopropylpyrimidine-5- carboxamide dr = 4.5:1 |
| 576 | 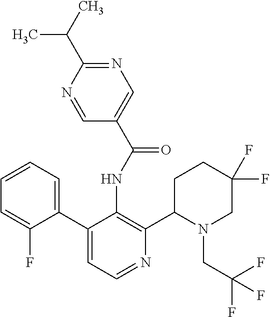 [SMILES: CC(C)c1ncc(C(=O)Nc2c(-c3ccccc3F)ccnc2C2CCC(F)(F)CN2CC(F)(F)F)cn1] | N-(2-(5,5-difluoro-1-(2,2,2- trifluoroethyl)piperidin-2-yl)-4-(2- fluorophenyl)pyridin-3-yl)-2- isopropylpyrimidine-5- carboxamide |
| 577 | 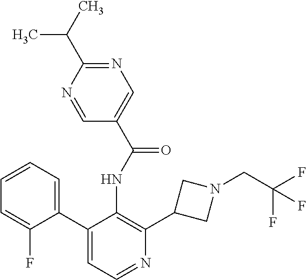 [SMILES: CC(C)c1ncc(C(=O)Nc2c(-c3ccccc3F)ccnc2C2CN(CC(F)(F)F)C2)cn1] | N-(4-(2-fluorophenyl)-2-(1-(2,2,2- trifluoroethyl)azetidin-3- yl)pyridin-3-yl)-2- isopropylpyrimidine-5- carboxamide |
| 578 | 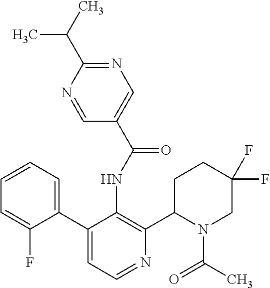 [SMILES: CC(=O)N1CC(F)(F)CCC1c1nccc(-c2ccccc2F)c1NC(=O)c1cnc(C(C)C)nc1] | N-(2-(1-acetyl-5,5- difluoropiperidin-2-yl)-4-(2- fluorophenyl)pyridin-3-yl)-2- isopropylpyrimidine-5- carboxamide |
| 579 | 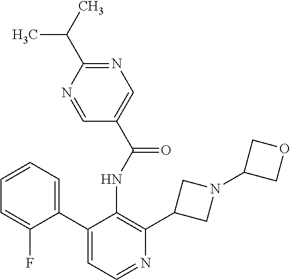 [SMILES: CC(C)c1ncc(C(=O)Nc2c(-c3ccccc3F)ccnc2C2CN(C3COC3)C2)cn1] | N-(4-(2-fluorophenyl)-2-(1- (oxetan-3-yl)azetidin-3-yl)pyridin- 3-yl)-2-isopropylpyrimidine-5- carboxamide |
| 580 | 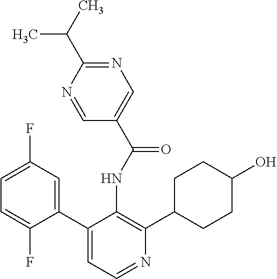 [SMILES: CC(C)c1ncc(C(=O)Nc2c(-c3cc(F)ccc3F)ccnc2C2CCC(O)CC2)cn1] | N-(4-(2,5-difluorophenyl)-2-(4- hydroxycyclohexyl)pyridin-3-yl)- 2-isopropylpyrimidine-5- carboxamide |
| 581 | 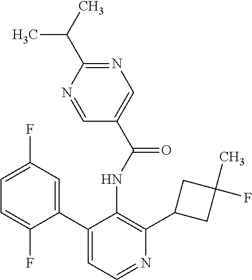 [SMILES: CC(C)c1ncc(C(=O)Nc2c(-c3cc(F)ccc3F)ccnc2C2CC(C)(F)C2)cn1] | N-(4-(2,5-difluorophenyl)-2-(3- fluoro-3-methylcyclobutyl)pyridin- 3-yl)-2-isopropylpyrimidine-5- carboxamide P1 |
| 582 | 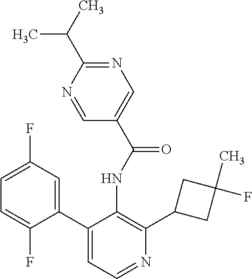 [SMILES: CC(C)c1ncc(C(=O)Nc2c(-c3cc(F)ccc3F)ccnc2C2CC(C)(F)C2)cn1] | N-(4-(2,5-difluorophenyl)-2-(3- fluoro-3-methylcyclobutyl)pyridin- 3-yl)-2-isopropylpyrimidine-5- carboxamide P2 |
| 583 | 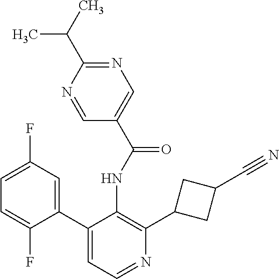 [SMILES: CC(C)c1ncc(C(=O)Nc2c(-c3cc(F)ccc3F)ccnc2C2CC(C#N)C2)cn1] | N-(2-(3-cyanocyclobutyl)-4-(2,5- difluorophenyl)pyridin-3-yl)-2- isopropylpyrimidine-5- carboxamide dr = 3:1 |
| 584 | 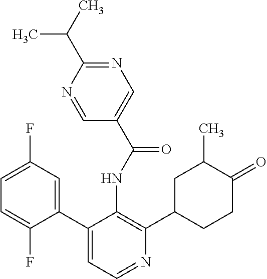 [SMILES: CC1CC(c2nccc(-c3cc(F)ccc3F)c2NC(=O)c2cnc(C(C)C)nc2)CCC1=O] | N-(4-(2,5-difluorophenyl)-2-(3- methyl-4-oxocyclohexyl)pyridin- 3-yl)-2-isopropylpyrimidine-5- carboxamide P1 |
| 585 | 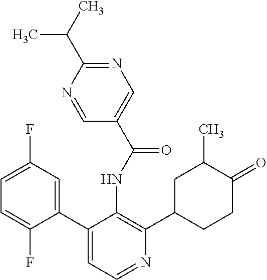 [SMILES: CC1CC(c2nccc(-c3cc(F)ccc3F)c2NC(=O)c2cnc(C(C)C)nc2)CCC1=O] | N-(4-(2,5-difluorophenyl)-2-(3- methyl-4-oxocyclohexyl)pyridin- 3-yl)-2-isopropylpyrimidine-5- carboxamide P2 |
| 586 | 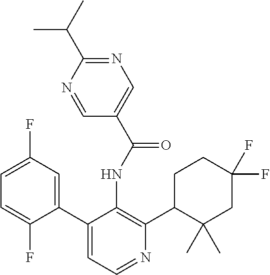 [SMILES: CC(C)c1ncc(C(=O)Nc2c(-c3cc(F)ccc3F)ccnc2C2CCC(F)(F)CC2(C)C)cn1] | N-(2-(4,4-difluoro-2,2- dimethylcyclohexyl)-4-(2,5- difluorophenyl)pyridin-3-yl)-2- isopropylpyrimidine-5- carboxamide |
| 587 | 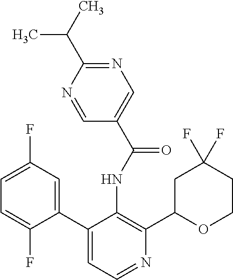 [SMILES: CC(C)c1ncc(C(=O)Nc2c(-c3cc(F)ccc3F)ccnc2C2CC(F)(F)CCO2)cn1] | N-(4-(2,5-difluorophenyl)-2-(4,4- difluorotetrahydro-2H-pyran-2- yl)pyridin-3-yl)-2- isopropylpyrimidine-5- carboxamide |
| 588 | 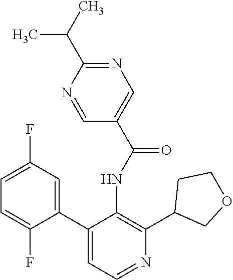 [SMILES: CC(C)c1ncc(C(=O)Nc2c(-c3cc(F)ccc3F)ccnc2C2CCOC2)cn1] | N-(4-(2,5-difluorophenyl)-2- (tetrahydrofuran-3-yl)pyridin-3- yl)-2-isopropylpyrimidine-5- carboxamide |
| 589 | 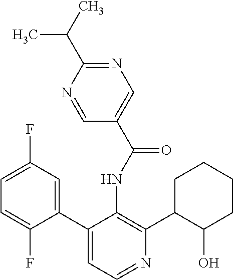 [SMILES: CC(C)c1ncc(C(=O)Nc2c(-c3cc(F)ccc3F)ccnc2C2CCCCC2O)cn1] | N-(4-(2,5-difluorophenyl)-2-(2- hydroxycyclohexyl)pyridin-3-yl)- 2-isopropylpyrimidine-5- carboxamide P1 |
| 590 | 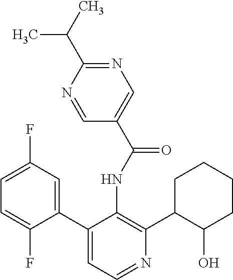 [SMILES: CC(C)c1ncc(C(=O)Nc2c(-c3cc(F)ccc3F)ccnc2C2CCCCC2O)cn1] | N-(4-(2,5-difluorophenyl)-2-(2- hydroxycyclohexyl)pyridin-3-yl)- 2-isopropylpyrimidine-5- carboxamide P2 |
| 591 | 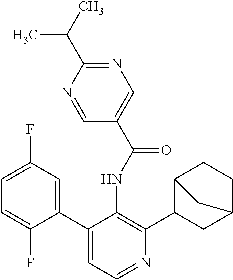 [SMILES: CC(C)c1ncc(C(=O)Nc2c(-c3cc(F)ccc3F)ccnc2C2CC3CCC2C3)cn1] | N-(2-(bicyclo[2.2.1]heptan-2-yl)- 4-(2,5-difluorophenyl)pyridin-3- yl)-2-isopropylpyrimidine-5- carboxamide |
| 592 | 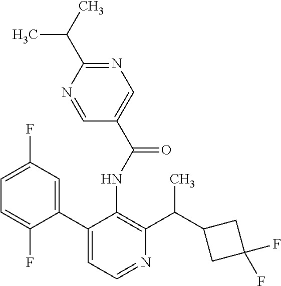 [SMILES: CC(C)c1ncc(C(=O)Nc2c(-c3cc(F)ccc3F)ccnc2C(C)C2CC(F)(F)C2)cn1] | N-(2-(1-(3,3- difluorocyclobutyl)ethyl)-4-(2,5- difluorophenyl)pyridin-3-yl)-2- isopropylpyrimidine-5- carboxamide |
| 593 | 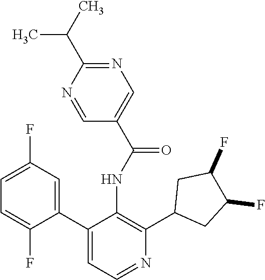 [SMILES: CC(C)c1ncc(C(=O)Nc2c(-c3cc(F)ccc3F)ccnc2C2C[C@@H](F)[C@@H](F)C2)cn1] | N-(2-(anti-3,4- difluorocyclopentyl)-4-(2,5- difluorophenyl)pyridin-3-yl)-2- isopropylpyrimidine-5- carboxamide |
| 594 | 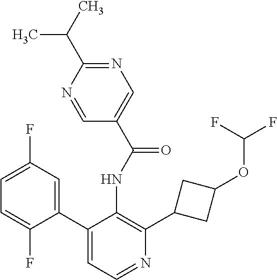 [SMILES: CC(C)c1ncc(C(=O)Nc2c(-c3cc(F)ccc3F)ccnc2C2CC(OC(F)F)C2)cn1] | N-(2-(3- (difluoromethoxy)cyclobutyl)-4- (2,5-difluorophenyl)pyridin-3-yl)- 2-isopropylpyrimidine-5- carboxamide D1 |
| 595 | 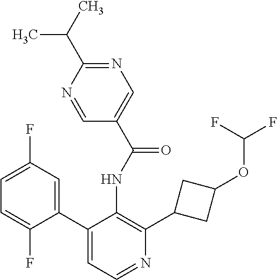 [SMILES: CC(C)c1ncc(C(=O)Nc2c(-c3cc(F)ccc3F)ccnc2C2CC(OC(F)F)C2)cn1] | N-(2-(3- (difluoromethoxy)cyclobutyl)-4- (2,5-difluorophenyl)pyridin-3-yl)- 2-isopropylpyrimidine-5- carboxamide D2 |
| 596 | 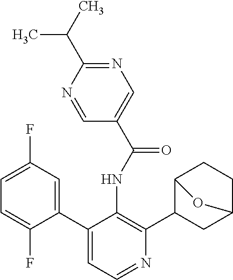 [SMILES: CC(C)c1ncc(C(=O)Nc2c(-c3cc(F)ccc3F)ccnc2C2CC3CCC2O3)cn1] | N-(2-(7-oxabicyclo[2.2.1]heptan- 2-yl)-4-(2,5- difluorophenyl)pyridin-3-yl)-2- isopropylpyrimidine-5- carboxamide |
| 597 | 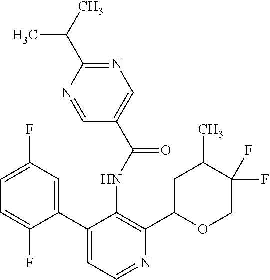 [SMILES: CC(C)c1ncc(C(=O)Nc2c(-c3cc(F)ccc3F)ccnc2C2CC(C)C(F)(F)CO2)cn1] | N-(2-(5,5-difluoro-4- methyltetrahydro-2H-pyran-2-yl)- 4-(2,5-difluorophenyl)pyridin-3- yl)-2-isopropylpyrimidine-5- carboxamide D1 |
| 598 | 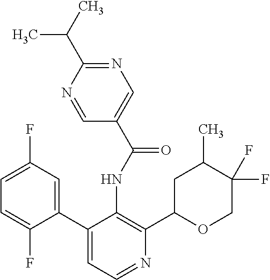 [SMILES: CC(C)c1ncc(C(=O)Nc2c(-c3cc(F)ccc3F)ccnc2C2CC(C)C(F)(F)CO2)cn1] | N-(2-(5,5-difluoro-4- methyltetrahydro-2H-pyran-2-yl)- 4-(2,5-difluorophenyl)pyridin-3- yl)-2-isopropylpyrimidine-5- carboxamide D2 |
| 599 | 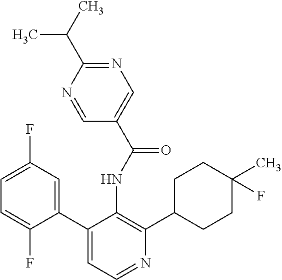 [SMILES: CC(C)c1ncc(C(=O)Nc2c(-c3cc(F)ccc3F)ccnc2C2CCC(C)(F)CC2)cn1] | N-(4-(2,5-difluorophenyl)-2-(4- fluoro-4- methylcyclohexyl)pyridin-3-yl)-2- isopropylpyrimidine-5- carboxamide D1 |
| 600 | 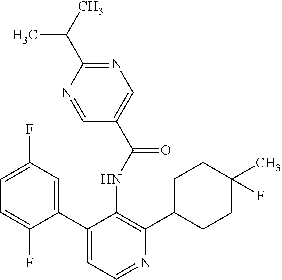 [SMILES: CC(C)c1ncc(C(=O)Nc2c(-c3cc(F)ccc3F)ccnc2C2CCC(C)(F)CC2)cn1] | N-(4-(2,5-difluorophenyl)-2-(4- fluoro-4- methylcyclohexyl)pyridin-3-yl)-2- isopropylpyrimidine-5- carboxamide D2 |
| 601 | 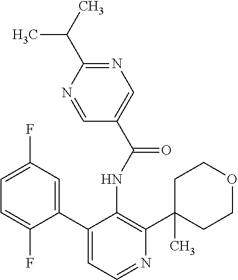 [SMILES: CC(C)c1ncc(C(=O)Nc2c(-c3cc(F)ccc3F)ccnc2C2(C)CCOCC2)cn1] | N-(4-(2,5-difluorophenyl)-2-(4- methyltetrahydro-2H-pyran-4- yl)pyridin-3-yl)-2- isopropylpyrimidine-5- carboxamide |
| 602 | 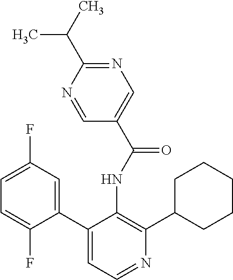 [SMILES: CC(C)c1ncc(C(=O)Nc2c(-c3cc(F)ccc3F)ccnc2C2CCCCC2)cn1] | N-(2-cyclohexyl-4-(2,5- difluorophenyl)pyridin-3-yl)-2- isopropylpyrimidine-5- carboxamide |
| 603 | 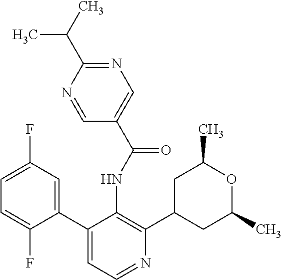 [SMILES: CC(C)c1ncc(C(=O)Nc2c(-c3cc(F)ccc3F)ccnc2C2C[C@@H](C)O[C@@H](C)C2)cn1] | N-(4-(2,5-difluorophenyl)-2- ((2R,6S)-2,6-dimethyltetrahydro- 2H-pyran-4-yl)pyridin-3-yl)-2- isopropylpyrimidine-5- carboxamide D1 |
| 604 | 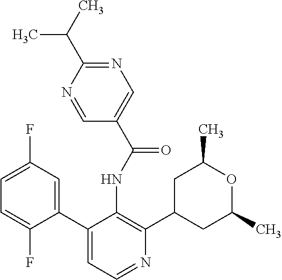 [SMILES: CC(C)c1ncc(C(=O)Nc2c(-c3cc(F)ccc3F)ccnc2C2C[C@@H](C)O[C@@H](C)C2)cn1] | N-(4-(2,5-difluorophenyl)-2- ((2R,6S)-2,6-dimethyltetrahydro- 2H-pyran-4-yl)pyridin-3-yl)-2- isopropylpyrimidine-5- carboxamide D2 |
| 605 | 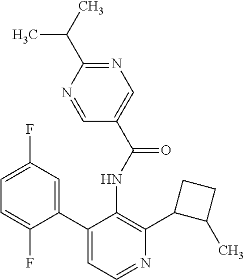 [SMILES: CC(C)c1ncc(C(=O)Nc2c(-c3cc(F)ccc3F)ccnc2C2CCC2C)cn1] | N-(4-(2,5-difluorophenyl)-2-(2- methylcyclobutyl)pyridin-3-yl)-2- isopropylpyrimidine-5- carboxamide |
| 606 | 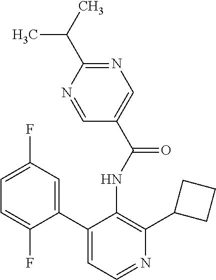 [SMILES: CC(C)c1ncc(C(=O)Nc2c(-c3cc(F)ccc3F)ccnc2C2CCC2)cn1] | N-(2-cyclobutyl-4-(2,5- difluorophenyl)pyridin-3-yl)-2- isopropylpyrimidine-5- carboxamide |
| 607 | 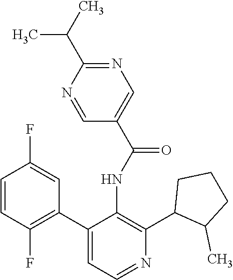 [SMILES: CC(C)c1ncc(C(=O)Nc2c(-c3cc(F)ccc3F)ccnc2C2CCCC2C)cn1] | N-(4-(2,5-difluorophenyl)-2-(2- methylcyclopentyl)pyridin-3-yl)-2- isopropylpyrimidine-5- carboxamide |
| 608 | 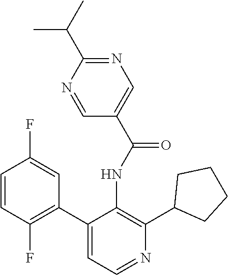 [SMILES: CC(C)c1ncc(C(=O)Nc2c(-c3cc(F)ccc3F)ccnc2C2CCCC2)cn1] | N-(2-cyclopentyl-4-(2,5- difluorophenyl)pyridin-3-yl)-2- isopropylpyrimidine-5- carboxamide |
| 609 | 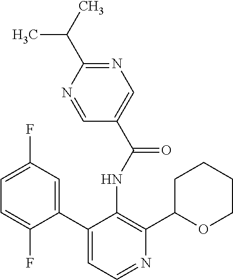 [SMILES: CC(C)c1ncc(C(=O)Nc2c(-c3cc(F)ccc3F)ccnc2C2CCCCO2)cn1] | N-(4-(2,5-difluorophenyl)-2- (tetrahydro-2H-pyran-2- yl)pyridin-3-yl)-2- isopropylpyrimidine-5- carboxamide |
| 610 | 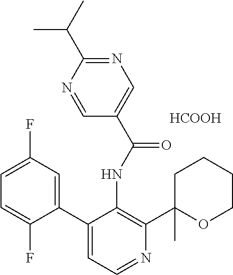 [SMILES: CC(C)c1ncc(C(=O)Nc2c(-c3cc(F)ccc3F)ccnc2C2(C)CCCCO2)cn1 ; "HCOOH"] | N-(4-(2,5-difluorophenyl)-2-(2- methyltetrahydro-2H-pyran-2- yl)pyridin-3-yl)-2- isopropylpyrimidine-5- carboxamide formic acid salt |
| 611 | 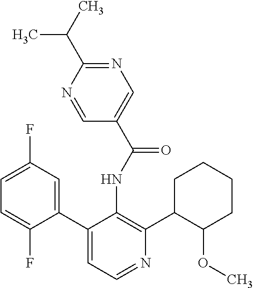 [SMILES: COC1CCCCC1c1nccc(-c2cc(F)ccc2F)c1NC(=O)c1cnc(C(C)C)nc1] | N-(4-(2,5-difluorophenyl)-2-(2- methoxycyclohexyl)pyridin-3-yl)- 2-isopropylpyrimidine-5- carboxamide |
| 612 | 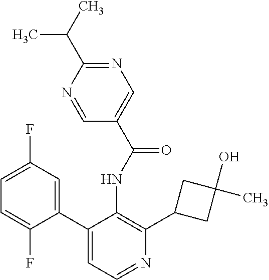 [SMILES: CC(C)c1ncc(C(=O)Nc2c(-c3cc(F)ccc3F)ccnc2C2CC(C)(O)C2)cn1] | N-(4-(2,5-difluorophenyl)-2-(3- hydroxy-3- methylcyclobutyl)pyridin-3-yl)-2- isopropylpyrimidine-5- carboxamide dr = 1:1 |
| 613 | 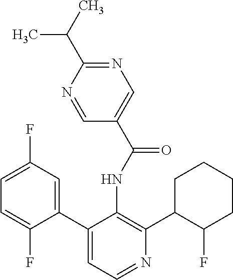 [SMILES: CC(C)c1ncc(C(=O)Nc2c(-c3cc(F)ccc3F)ccnc2C2CCCCC2F)cn1] | N-(4-(2,5-difluorophenyl)-2-(2- fluorocyclohexyl)pyridin-3-yl)-2- isopropylpyrimidine-5- carboxamide |
| 614 | 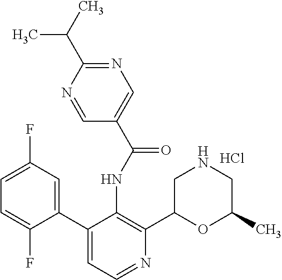 [SMILES: CC(C)c1ncc(C(=O)Nc2c(-c3cc(F)ccc3F)ccnc2C2CNC[C@@H](C)O2)cn1 ; Cl] | N-(4-(2,5-difluorophenyl)-2- ((6R)-6-methylmorpholin-2- yl)pyridin-3-yl)-2- isopropylpyrimidine-5- carboxamide hydrochloric formic acid mixed salt |
| 615 | 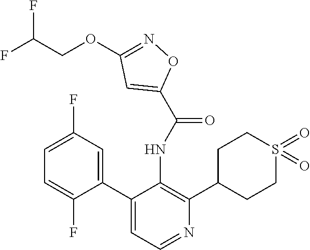 [SMILES: O=C(Nc1c(-c2cc(F)ccc2F)ccnc1C1CCS(=O)(=O)CC1)c1cc(OCC(F)F)no1] | 3-(2,2-difluoroethoxy)-N-(4-(2,5- difluorophenyl)-2-(1,1- dioxidotetrahydro-2H-thiopyran- 4-yl)pyridin-3-yl)isoxazole-5- carboxamide |
| 616 | 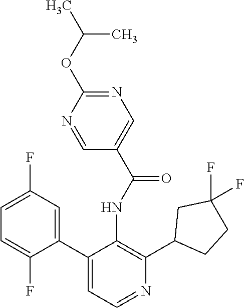 [SMILES: CC(C)Oc1ncc(C(=O)Nc2c(-c3cc(F)ccc3F)ccnc2C2CCC(F)(F)C2)cn1] | N-(2-(3,3-difluorocyclopentyl)-4- (2,5-difluorophenyl)pyridin-3-yl)- 2-isopropoxypyrimidine-5- carboxamide |
| 617 | 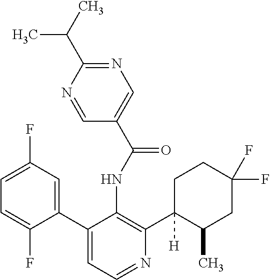 [SMILES: CC(C)c1ncc(C(=O)Nc2c(-c3cc(F)ccc3F)ccnc2[C@H]2CCC(F)(F)C[C@H]2C)cn1] | N-(2-(syn-4,4-difluoro-2- methylcyclohexyl)-4-(2,5- difluorophenyl)pyridin-3-yl)-2- isopropylpyrimidine-5- carboxamide |
| 618 | 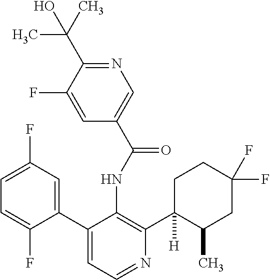 [SMILES: C[C@@H]1CC(F)(F)CC[C@@H]1c1nccc(-c2cc(F)ccc2F)c1NC(=O)c1cnc(C(C)(C)O)c(F)c1] | N-(2-(syn-4,4-difluoro-2- methylcyclohexyl)-4-(2,5- difluorophenyl)pyridin-3-yl)-5- fluoro-6-(2-hydroxypropan-2- yl)nicotinamide |
| 619 | 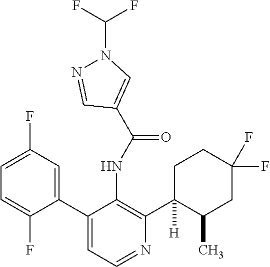 [SMILES: C[C@@H]1CC(F)(F)CC[C@@H]1c1nccc(-c2cc(F)ccc2F)c1NC(=O)c1cnn(C(F)F)c1] | N-(2-(syn-4,4-difluoro-2- methylcyclohexyl)-4-(2,5- difluorophenyl)pyridin-3-yl)-1- (difluoromethyl)-1H-pyrazole-4- carboxamide |
| 620 | 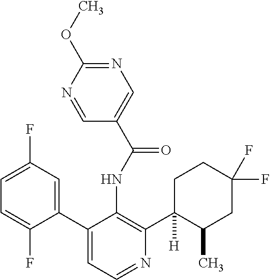 [SMILES: COc1ncc(C(=O)Nc2c(-c3cc(F)ccc3F)ccnc2[C@H]2CCC(F)(F)C[C@H]2C)cn1] | N-(2-(syn-4,4-difluoro-2- methylcyclohexyl)-4-(2,5- difluorophenyl)pyridin-3-yl)-2- methoxypyrimidine-5- carboxamide |
| 621 | 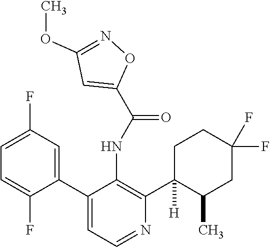 [SMILES: COc1cc(C(=O)Nc2c(-c3cc(F)ccc3F)ccnc2[C@H]2CCC(F)(F)C[C@H]2C)on1] | N-(2-syn-4,4-difluoro-2- methylcyclohexyl)-4-(2,5- difluorophenyl)pyridin-3-yl)-3- methoxyisoxazole-5- carboxamide |
| 622 | 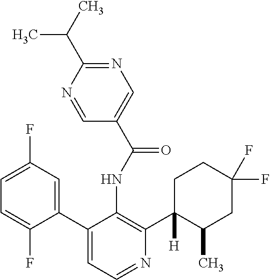 [SMILES: CC(C)c1ncc(C(=O)Nc2c(-c3cc(F)ccc3F)ccnc2[C@@H]2CCC(F)(F)C[C@H]2C)cn1] | N-(2-(anti-4,4-difluoro-2- methylcyclohexyl)-4-(2,5- difluorophenyl)pyridin-3-yl)-2- isopropylpyrimidine-5- carboxamide |
| 623 | 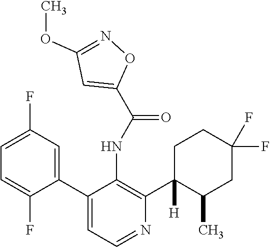 [SMILES: COc1cc(C(=O)Nc2c(-c3cc(F)ccc3F)ccnc2[C@@H]2CCC(F)(F)C[C@H]2C)on1] | N-(2-(anti-4,4-difluoro-2- methylcyclohexyl)-4-(2,5- difluorophenyl)pyridin-3-yl)-3- methoxyisoxazole-5- carboxamide |
| 624 | 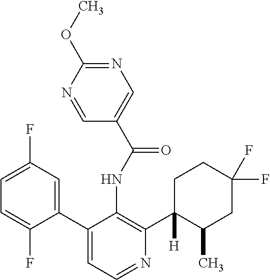 [SMILES: COc1ncc(C(=O)Nc2c(-c3cc(F)ccc3F)ccnc2[C@@H]2CCC(F)(F)C[C@H]2C)cn1] | N-(2-(anti-4,4-difluoro-2- methylcyclohexyl)-4-(2,5- difluorophenyl)pyridin-3-yl)-2- methoxypyrimidine-5- carboxamide |
| 625 | 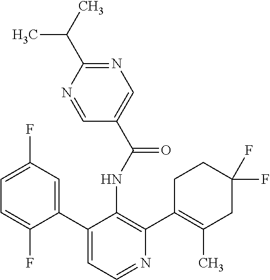 [SMILES: CC1=C(c2nccc(-c3cc(F)ccc3F)c2NC(=O)c2cnc(C(C)C)nc2)CCC(F)(F)C1] | N-(2-(4,4-difluoro-2- methylcyclohex-1-en-1-yl)-4- (2,5-difluorophenyl)pyridin-3-yl)- 2-isopropylpyrimidine-5- carboxamide |
| 626 | 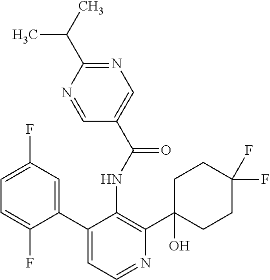 [SMILES: CC(C)c1ncc(C(=O)Nc2c(-c3cc(F)ccc3F)ccnc2C2(O)CCC(F)(F)CC2)cn1] | N-(2-(4,4-difluoro-1- hydroxycyclohexyl)-4-(2,5- difluorophenyl)pyridin-3-yl)-2- isopropylpyrimidine-5- carboxamide |
| 627 | 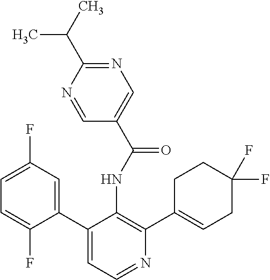 [SMILES: CC(C)c1ncc(C(=O)Nc2c(-c3cc(F)ccc3F)ccnc2C2=CCC(F)(F)CC2)cn1] | N-(2-(4,4-difluorocyclohex-1-en- 1-yl)-4-(2,5- difluorophenyl)pyridin-3-yl)-2- isopropylpyrimidine-5- carboxamide |
| 628 | 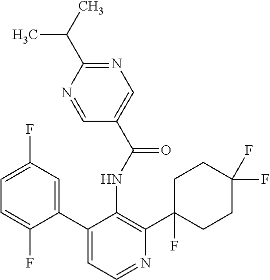 [SMILES: CC(C)c1ncc(C(=O)Nc2c(-c3cc(F)ccc3F)ccnc2C2(F)CCC(F)(F)CC2)cn1] | N-(4-(2,5-difluorophenyl)-2- (1,4,4-trifluorocyclohexyl)pyridin- 3-yl)-2-isopropylpyrimidine-5- carboxamide |
| 629 | 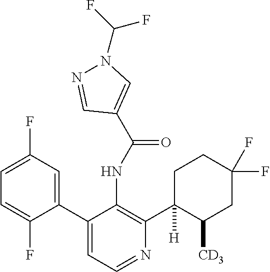 [SMILES: [2H]C([2H])([2H])[C@@H]1CC(F)(F)CC[C@@H]1c1nccc(-c2cc(F)ccc2F)c1NC(=O)c1cnn(C(F)F)c1] | N-(2-(syn-4,4-difluoro-2-(methyl- d₃)cyclohexyl)-4-(2,5- difluorophenyl)pyridin-3-yl)-1- (difluoromethyl)-1H-pyrazole-4- carboxamide |
| 630 | 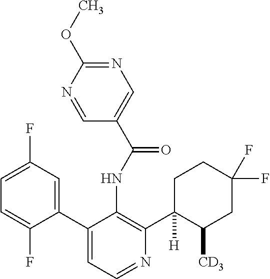 [SMILES: [2H]C([2H])([2H])[C@@H]1CC(F)(F)CC[C@@H]1c1nccc(-c2cc(F)ccc2F)c1NC(=O)c1cnc(OC)nc1] | N-(2-(syn-4,4-difluoro-2-(methyl- d₃)cyclohexyl)-4-(2,5- difluorophenyl)pyridin-3-yl)-2- methoxypyrimidine-5- carboxamide |
| 631 | 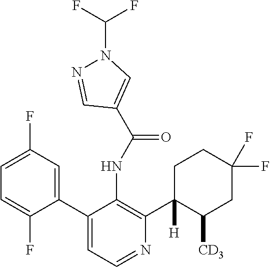 [SMILES: [2H]C([2H])([2H])[C@@H]1CC(F)(F)CC[C@H]1c1nccc(-c2cc(F)ccc2F)c1NC(=O)c1cnn(C(F)F)c1] | (2-(anti-4,4-difluoro-2-(methyl- d₃)cyclohexyl)-4-(2,5- difluorophenyl)pyridin-3-yl)-1- (difluoromethyl)-1H-pyrazole-4- carboxamide |
| 632 | 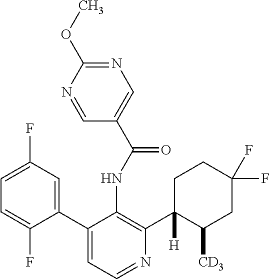 [SMILES: [2H]C([2H])([2H])[C@@H]1CC(F)(F)CC[C@H]1c1nccc(-c2cc(F)ccc2F)c1NC(=O)c1cnc(OC)nc1] | N-(2-(anti-4,4-difluoro-2-(methyl- d₃)cyclohexyl)-4-(2,5- difluorophenyl)pyridin-3-yl)-2- methoxypyrimidine-5- carboxamide |
| 633 | 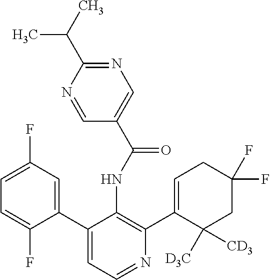 [SMILES: [2H]C([2H])([2H])C1(C([2H])([2H])[2H])CC(F)(F)CC=C1c1nccc(-c2cc(F)ccc2F)c1NC(=O)c1cnc(C(C)C)nc1] | N-(2-(4,4-difluoro-6,6-bis(methyl- d₃)cyclohex-1-en-1-yl)-4-(2,5- difluorophenyl)pyridin-3-yl)-2- isopropylpyrimidine-5- carboxamide |
| 634 | 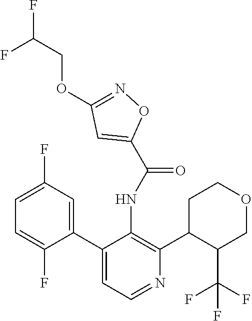 [SMILES: O=C(Nc1c(-c2cc(F)ccc2F)ccnc1C1CCOCC1C(F)(F)F)c1cc(OCC(F)F)no1] | 3-(2,2-difluoroethoxy)-N-(4-(2,5- difluorophenyl)-2-(3- (trifluoromethyl)tetrahydro-2H- pyran-4-yl)pyridin-3-yl)isoxazole- 5-carboxamide |
| 635 | 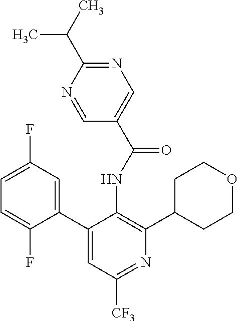 [SMILES: CC(C)c1ncc(C(=O)Nc2c(-c3cc(F)ccc3F)cc(C(F)(F)F)nc2C2CCOCC2)cn1] | N-(4-(2,5-difluorophenyl)-2- (tetrahydro-2H-pyran-4-yl)-6- (trifluoromethyl)pyridin-3-yl)-2- isopropylpyrimidine-5- carboxamide |
| 636 | 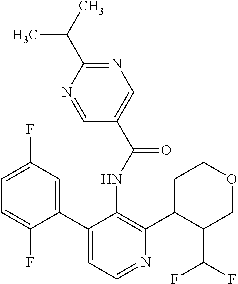 [SMILES: CC(C)c1ncc(C(=O)Nc2c(-c3cc(F)ccc3F)ccnc2C2CCOCC2C(F)F)cn1] | N-(2-(3- (difluoromethyl)tetrahydro-2H- pyran-4-yl)-4-(2,5- difluorophenyl)pyridin-3-yl)-2- isopropylpyrimidine-5- carboxamide |
| 637 | 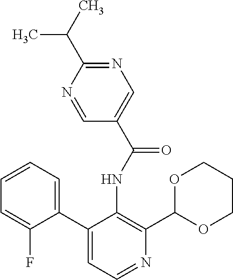 [SMILES: CC(C)c1ncc(C(=O)Nc2c(-c3ccccc3F)ccnc2C2OCCCO2)cn1] | N-(2-(1,3-dioxan-2-yl)-4-(2- fluorophenyl)pyridin-3-yl)-2- isopropylpyrimidine-5- carboxamide |
| 638 | 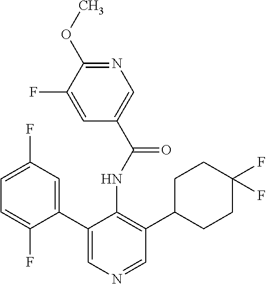 [SMILES: COc1ncc(C(=O)Nc2c(-c3cc(F)ccc3F)cncc2C2CCC(F)(F)CC2)cc1F] | N-(3-(4,4-difluorocyclohexyl)-5- (2,5-difluorophenyl)pyridin-4-yl)- 5-fluoro-6-methoxynicotinamide |
| 639 | 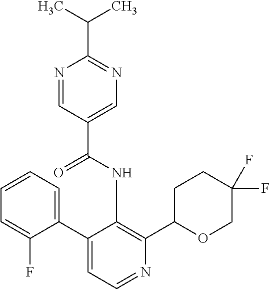 [SMILES: CC(C)c1ncc(C(=O)Nc2c(-c3ccccc3F)ccnc2C2CCC(F)(F)CO2)cn1] | N-(2-(5,5-difluorotetrahydro-2H- pyran-2-yl)-4-(2- fluorophenyl)pyridin-3-yl)-2- isopropylpyrimidine-5- carboxamide P1 |
| 640 | 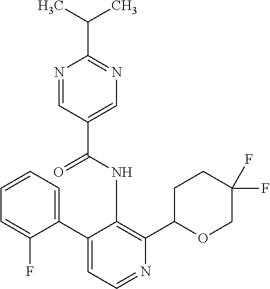 [SMILES: CC(C)c1ncc(C(=O)Nc2c(-c3ccccc3F)ccnc2C2CCC(F)(F)CO2)cn1] | N-(2-(5,5-difluorotetrahydro-2H- pyran-2-yl)-4-(2- fluorophenyl)pyridin-3-yl)-2- isopropylpyrimidine-5- carboxamide P2 |
| 641 | 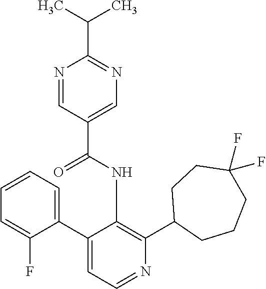 [SMILES: CC(C)c1ncc(C(=O)Nc2c(-c3ccccc3F)ccnc2C2CCCC(F)(F)CC2)cn1] | N-(2-(4,4-difluorocycloheptyl)-4- (2-fluorophenyl)pyridin-3-yl)-2- isopropylpyrimidine-5- carboxamide P1 |
| 642 | 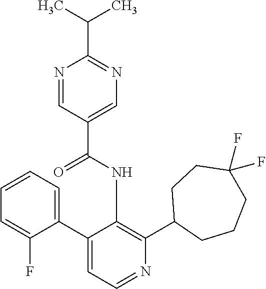 [SMILES: CC(C)c1ncc(C(=O)Nc2c(-c3ccccc3F)ccnc2C2CCCC(F)(F)CC2)cn1] | N-(2-(4,4-difluorocycloheptyl)-4- (2-fluorophenyl)pyridin-3-yl)-2- isopropylpyrimidine-5- carboxamide P2 |
| 643 | 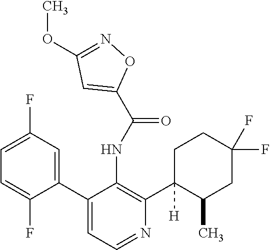 [SMILES: COc1cc(C(=O)Nc2c(-c3cc(F)ccc3F)ccnc2[C@H]2CCC(F)(F)C[C@H]2C)on1] | N-(2-(syn-4,4-difluoro-2- methylcyclohexyl)-4-(2,5- difluorophenyl)pyridin-3-yl)-3- methoxyisoxazole-5- carboxamide P1 |
| 644 | 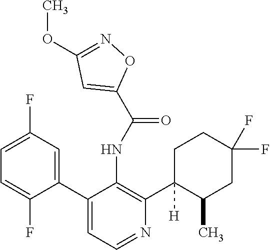 [SMILES: COc1cc(C(=O)Nc2c(-c3cc(F)ccc3F)ccnc2[C@H]2CCC(F)(F)C[C@H]2C)on1] | N-(2-(syn-4,4-difluoro-2- methylcyclohexyl)-4-(2,5- difluorophenyl)pyridin-3-yl)-3- methoxyisoxazole-5- carboxamide P2 |
| 645 | 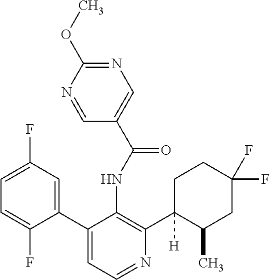 [SMILES: COc1ncc(C(=O)Nc2c(-c3cc(F)ccc3F)ccnc2[C@H]2CCC(F)(F)C[C@H]2C)cn1] | N-(2-(syn-4,4-difluoro-2- methylcyclohexyl)-4-(2,5- difluorophenyl)pyridin-3-yl)-2- methoxypyrimidine-5- carboxamide P1 |
| 646 | 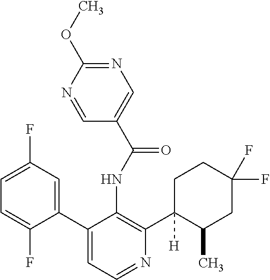 [SMILES: COc1ncc(C(=O)Nc2c(-c3cc(F)ccc3F)ccnc2[C@H]2CCC(F)(F)C[C@H]2C)cn1] | N-(2-(syn-4,4-difluoro-2- methylcyclohexyl)-4-(2,5- difluorophenyl)pyridin-3-yl)-2- methoxypyrimidine-5- carboxamide P2 |
| 647 | 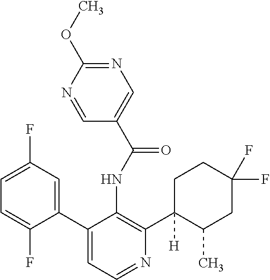 [SMILES: COc1ncc(C(=O)Nc2c(-c3cc(F)ccc3F)ccnc2[C@H]2CCC(F)(F)C[C@@H]2C)cn1] | N-(2-(anti-4,4-difluoro-2- methylcyclohexyl)-4-(2,5- difluorophenyl)pyridin-3-yl)-2- methoxypyrimidine-5- carboxamide P1 |
| 648 | 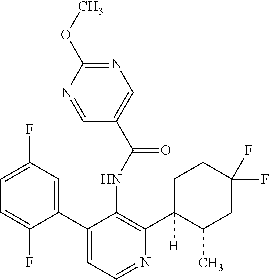 [SMILES: COc1ncc(C(=O)Nc2c(-c3cc(F)ccc3F)ccnc2[C@H]2CCC(F)(F)C[C@@H]2C)cn1] | N-(2-(anti-4,4-difluoro-2- methylcyclohexyl)-4-(2,5- difluorophenyl)pyridin-3-yl)-2- methoxypyrimidine-5- carboxamide P2 |
| 649 | 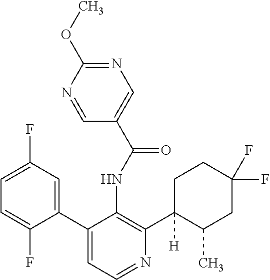 [SMILES: COc1ncc(C(=O)Nc2c(-c3cc(F)ccc3F)ccnc2[C@H]2CCC(F)(F)C[C@@H]2C)cn1] | N-(2-(anti-4,4-difluoro-2- methylcyclohexyl)-4-(2,5- difluorophenyl)pyridin-3-yl)-3- methoxyisoxazole-5- carboxamide P1 |
| 650 | 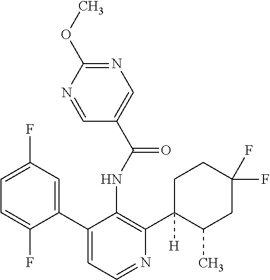 [SMILES: COc1ncc(C(=O)Nc2c(-c3cc(F)ccc3F)ccnc2[C@H]2CCC(F)(F)C[C@@H]2C)cn1] | N-(2-(anti-4,4-difluoro-2- methylcyclohexyl)-4-(2,5- difluorophenyl)pyridin-3-yl)-3- methoxyisoxazole-5- carboxamide P2 |
| 651 | 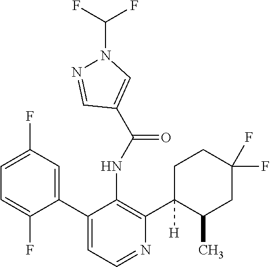 [SMILES: C[C@@H]1CC(F)(F)CC[C@@H]1c1nccc(-c2cc(F)ccc2F)c1NC(=O)c1cnn(C(F)F)c1] | N-(2-(syn-4,4-difluoro-2- methylcyclohexyl)-4-(2,5- difluorophenyl)pyridin-3-yl)-1- (difluoromethyl)-1H-pyrazole-4- carboxamide P1 |
| 652 | 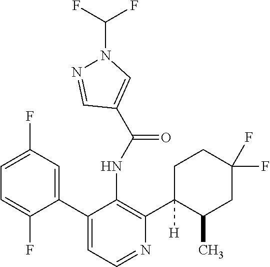 [SMILES: C[C@@H]1CC(F)(F)CC[C@@H]1c1nccc(-c2cc(F)ccc2F)c1NC(=O)c1cnn(C(F)F)c1] | N-(2-(syn-4,4-difluoro-2- methylcyclohexyl)-4-(2,5- difluorophenyl)pyridin-3-yl)-1- (difluoromethyl)-1H-pyrazole-4- carboxamide P2 |
| 653 | 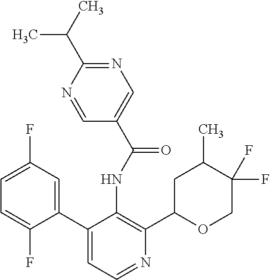 [SMILES: CC(C)c1ncc(C(=O)Nc2c(-c3cc(F)ccc3F)ccnc2C2CC(C)C(F)(F)CO2)cn1] | N-(2-(5,5-difluoro-4- methyltetrahydro-2H-pyran-2-yl)- 4-(2,5-difluorophenyl)pyridin-3- yl)-2-isopropylpyrimidine-5- carboxamide P1, D1 |
| 654 | 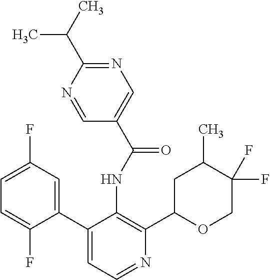 [SMILES: CC(C)c1ncc(C(=O)Nc2c(-c3cc(F)ccc3F)ccnc2C2CC(C)C(F)(F)CO2)cn1] | N-(2-(5,5-difluoro-4- methyltetrahydro-2H-pyran-2-yl)- 4-(2,5-difluorophenyl)pyridin-3- yl)-2-isopropylpyrimidine-5- carboxamide P2, D1 |
| 655 | 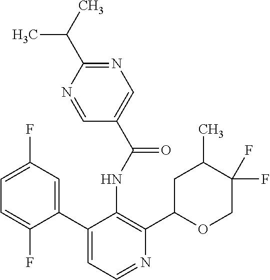 [SMILES: CC(C)c1ncc(C(=O)Nc2c(-c3cc(F)ccc3F)ccnc2C2CC(C)C(F)(F)CO2)cn1] | N-(2-(5,5-difluoro-4- methyltetrahydro-2H-pyran-2-yl)- 4-(2,5-difluorophenyl)pyridin-3- yl)-2-isopropylpyrimidine-5- carboxamide P1, D2 |
| 656 | 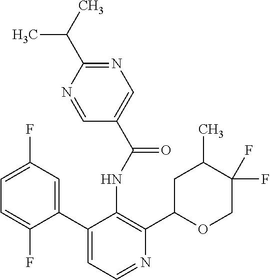 [SMILES: CC(C)c1ncc(C(=O)Nc2c(-c3cc(F)ccc3F)ccnc2C2CC(C)C(F)(F)CO2)cn1] | N-(2-(5,5-difluoro-4- methyltetrahydro-2H-pyran-2-yl)- 4-(2,5-difluorophenyl)pyridin-3- yl)-2-isopropylpyrimidine-5- carboxamide P2, D2 |
| 657 | 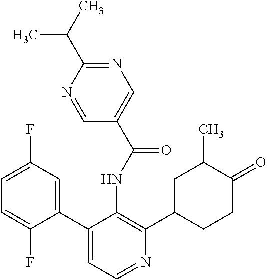 [SMILES: CC1CC(c2nccc(-c3cc(F)ccc3F)c2NC(=O)c2cnc(C(C)C)nc2)CCC1=O] | N-(4-(2,5-difluorophenyl)-2-(3- methyl-4-oxocyclohexyl)pyridin- 3-yl)-2-isopropylpyrimidine-5- carboxamide P1, D2 |
| 658 | 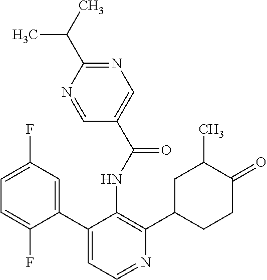 [SMILES: CC1CC(c2nccc(-c3cc(F)ccc3F)c2NC(=O)c2cnc(C(C)C)nc2)CCC1=O] | N-(4-(2,5-difluorophenyl)-2-(3- methyl-4-oxocyclohexyl)pyridin- 3-yl)-2-isopropylpyrimidine-5- carboxamide P2, D2 |
| 659 | 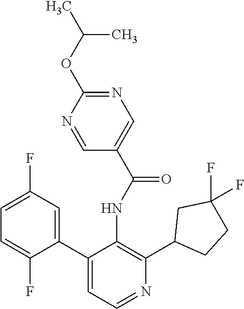 [SMILES: CC(C)Oc1ncc(C(=O)Nc2c(-c3cc(F)ccc3F)ccnc2C2CCC(F)(F)C2)cn1] | N-(2-(3,3-difluorocyclopentyl)-4- (2,5-difluorophenyl)pyridin-3-yl)- 2-isopropoxypyrimidine-5- carboxamide P1 |
| 660 | 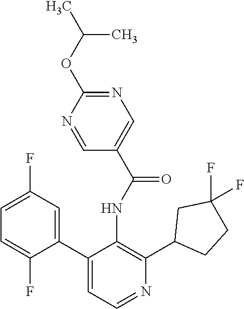 [SMILES: CC(C)Oc1ncc(C(=O)Nc2c(-c3cc(F)ccc3F)ccnc2C2CCC(F)(F)C2)cn1] | N-(2-(3,3-difluorocyclopenty)-4- (2,5-difluorophenyl)pyridin-3-yl)- 2-isopropoxypyrimidine-5- carboxamide P2 |
| 661 | 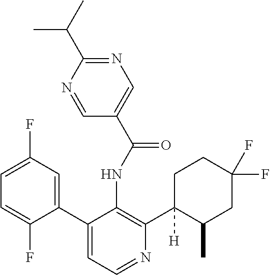 [SMILES: CC(C)c1ncc(C(=O)Nc2c(-c3cc(F)ccc3F)ccnc2[C@H]2CCC(F)(F)C[C@H]2C)cn1] | N-(2-(syn-4,4-difluoro-2- methylcyclohexyl)-4-(2,5- difluorophenyl)pyridin-3-yl)-2- isopropylpyrimidine-5- carboxamide P1 |
| 662 | 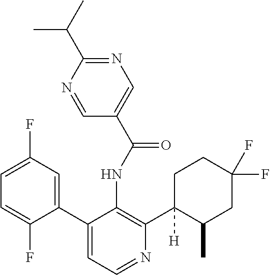 [SMILES: CC(C)c1ncc(C(=O)Nc2c(-c3cc(F)ccc3F)ccnc2[C@H]2CCC(F)(F)C[C@H]2C)cn1] | N-(2-(syn-4,4-difluoro-2- methylcyclohexyl)-4-(2,5- difluorophenyl)pyridin-3-yl)-2- isopropylpyrimidine-5- carboxamide P2 |
| 663 | 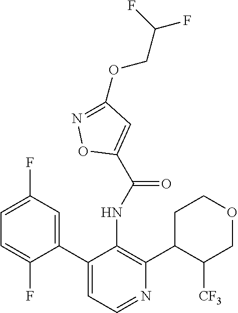 [SMILES: O=C(Nc1c(-c2cc(F)ccc2F)ccnc1C1CCOCC1C(F)(F)F)c1cc(OCC(F)F)no1] | 3-(2,2-difluoroethoxy)-N-(4-(2,5- difluorophenyl)-2-(3- (trifluoromethyl)tetrahydro-2H- pyran-4-yl)pyridin-3-yl)isoxazole- 5-carboxamide P1 |
| 664 | 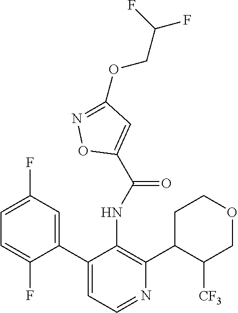 [SMILES: O=C(Nc1c(-c2cc(F)ccc2F)ccnc1C1CCOCC1C(F)(F)F)c1cc(OCC(F)F)no1] | 3-(2,2-difluoroethoxy)-N-(4-(2,5- difluorophenyl)-2-(3- (trifluoromethyl)tetrahydro-2H- pyran-4-yl)pyridin-3-yl)isoxazole- 5-carboxamide P2 |
| 665 | 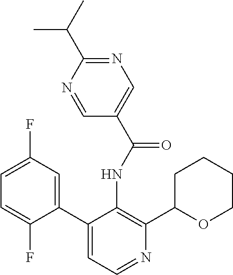 [SMILES: CC(C)c1ncc(C(=O)Nc2c(-c3cc(F)ccc3F)ccnc2C2CCCCO2)cn1] | N-(4-(2,5-difluorophenyl)-2- (tetrahydro-2H-pyran-2- yl)pyridin-3-yl)-2- isopropylpyrimidine-5- carboxamide P1 |
| 666 | 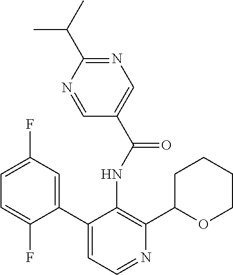 [SMILES: CC(C)c1ncc(C(=O)Nc2c(-c3cc(F)ccc3F)ccnc2C2CCCCO2)cn1] | N-(4-(2,5-difluorophenyl)-2- (tetrahydro-2H-pyran-2- yl)pyridin-3-yl)-2- isopropylpyrimidine-5- carboxamide P2 |
| 667 | 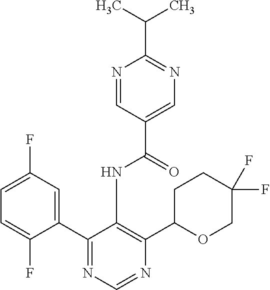 [SMILES: CC(C)c1ncc(C(=O)Nc2c(-c3cc(F)ccc3F)ncnc2C2CCC(F)(F)CO2)cn1] | N-(4-(2,5-difluorophenyl)-6-(5,5- difluorotetrahydro-2H-pyran-2- yl)pyrimidin-5-yl)-2- isopropylpyrimidine-5- carboxamide |
| 668 | 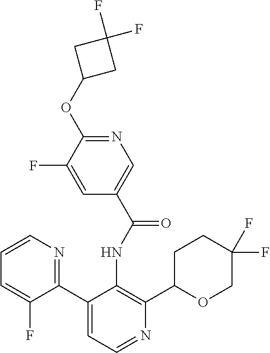 [SMILES: O=C(Nc1c(-c2ncccc2F)ccnc1C1CCC(F)(F)CO1)c1cnc(OC2CC(F)(F)C2)c(F)c1] | rac-6-(3,3-difluorocyclobutoxy)- N-(2′-(5,5-difluorotetrahydro-2H- pyran-2-yl)-3-fluoro-[2,4′- bipyridin]-3′-yl)-5- fluoronicotinamide |
| 669 | 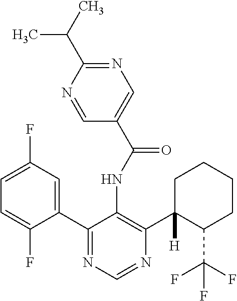 [SMILES: CC(C)c1ncc(C(=O)Nc2c(-c3cc(F)ccc3F)ncnc2[C@@H]2CCCC[C@@H]2C(F)(F)F)cn1] | rac-N-(4-(2,5-difluorophenyl)-6- (2-(trifluoromethyl)cyclohexyl) pyrimidin-5-yl)-2-isopropylpyrimidine- 5-carboxamide |
| 670 | 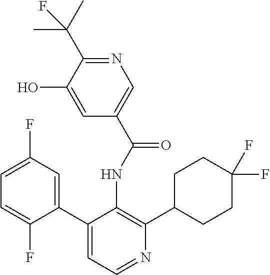 [SMILES: CC(C)(F)c1ncc(C(=O)Nc2c(-c3cc(F)ccc3F)ccnc2C2CCC(F)(F)CC2)cc1O] | N-(2-(4,4-difluorocyclohexyl)-4- (2,5-difluorophenyl)pyridin-3-yl)- 6-(2-fluoropropan-2-yl)-5- hydroxynicotinamide |
| 671 | 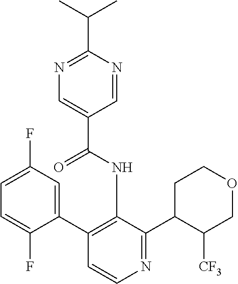 [SMILES: CC(C)c1ncc(C(=O)Nc2c(-c3cc(F)ccc3F)ccnc2C2CCOCC2C(F)(F)F)cn1] | N-(4-(2,5-difluorophenyl)-2-(3- (trifluoromethyl)tetrahydro-2H- pyran-4-yl)pyridin-3-yl)-2- isopropylpyrimidine-5- carboxamide (racemic mixture) |
| 672 | 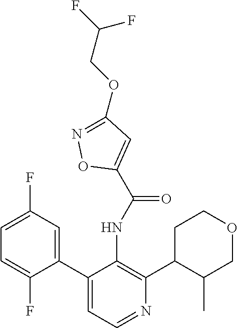 [SMILES: CC1COCCC1c1nccc(-c2cc(F)ccc2F)c1NC(=O)c1cc(OCC(F)F)no1] | 3-(2,2-difluoroethoxy)-N-(4-(2,5- difluorophenyl)-2-(3- methyltetrahydro-2H-pyran-4- yl)pyridin-3-yl)isoxazole-5- carboxamide (racemic mixture) |
| 673 | 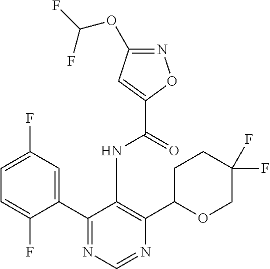 [SMILES: O=C(Nc1c(-c2cc(F)ccc2F)ncnc1C1CCC(F)(F)CO1)c1cc(OC(F)F)no1] | 3-(difluoromethoxy)-N-(4-(2,5- difluorophenyl)-6-(5,5- difluorotetrahydro-2H-pyran-2- yl)pyrimidin-5-yl)isoxazole-5- carboxamide P1 |
| 674 | 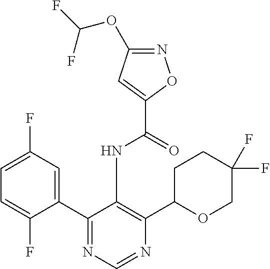 [SMILES: O=C(Nc1c(-c2cc(F)ccc2F)ncnc1C1CCC(F)(F)CO1)c1cc(OC(F)F)no1] | 3-(difluoromethoxy)-N-(4-(2,5- difluorophenyl)-6-(5,5- difluorotetrahydro-2H-pyran-2- yl)pyrimidin-5-yl)isoxazole-5- carboxamide P2 |
| 675 | 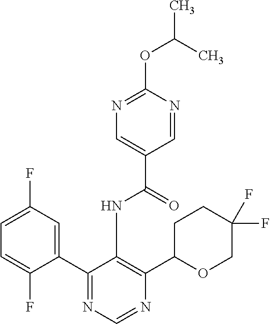 [SMILES: CC(C)Oc1ncc(C(=O)Nc2c(-c3cc(F)ccc3F)ncnc2C2CCC(F)(F)CO2)cn1] | N-(4-(2,5-difluorophenyl)-6-(5,5- difluorotetrahydro-2H-pyran-2- yl)pyrimidin-5-yl)-2- isopropoxypyrimidine-5- carboxamide |
| 676 | 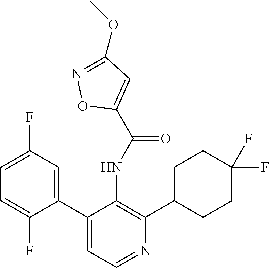 [SMILES: COc1cc(C(=O)Nc2c(-c3cc(F)ccc3F)ccnc2C2CCC(F)(F)CC2)on1] | N-(2-(4,4-difluorocyclohexyl)-4- (2,5-difluorophenyl)pyridin-3-yl)- 3-methoxyisoxazole-5- carboxamide |
| 677 | 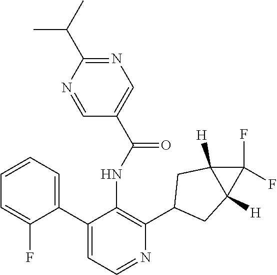 [SMILES: CC(C)c1ncc(C(=O)Nc2c(-c3ccccc3F)ccnc2C2C[C@@H]3[C@H](C2)C3(F)F)cn1] | N-[2-[(1R,5S)-6,6-difluoro-3- bicyclo[3.1.0]hexanyl]-4-(2- fluorophenyl)-3-pyridyl]-2- isopropyl-pyrimidine-5- carboxamide P1 |
| 678 | 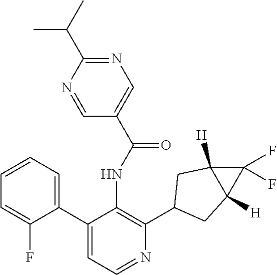 [SMILES: CC(C)c1ncc(C(=O)Nc2c(-c3ccccc3F)ccnc2C2C[C@@H]3[C@H](C2)C3(F)F)cn1] | N-[2-[(1R,5S)-6,6-difluoro-3- bicyclo[3.1.0]hexanyl]-4-(2- fluorophenyl)-3-pyridyl]-2- isopropyl-pyrimidine-5- carboxamide P2 |
| 679 | 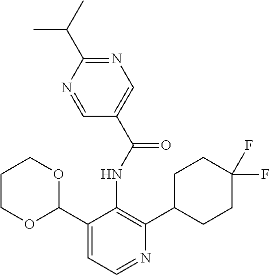 [SMILES: CC(C)c1ncc(C(=O)Nc2c(C3OCCCO3)ccnc2C2CCC(F)(F)CC2)cn1] | N-(2-(4,4-difluorocyclohexyl)-4- (1,3-dioxan-2-yl)pyridin-3-yl)-2- isopropylpyrimidine-5- carboxamide |

Another embodiment of the disclosure is a pharmaceutical composition comprising one or more pharmaceutically acceptable excipient(s) and a therapeutically effective amount of a compound of formula (I) or (II), as described above in the Brief Summary, as a stereoisomer, enantiomer, or tautomer thereof or mixtures thereof; or a pharmaceutically acceptable salt, solvate, or prodrug thereof.

Another embodiment of the disclosure is a method of treating a disease or condition in a mammal modulated by a voltage-gated sodium channel, wherein the method comprises administering to a mammal in need thereof a therapeutically effective amount of a compound of formula (I) or (II), as described above in the Summary of the disclosure, as a stereoisomer, enantiomer, or tautomer thereof or mixtures thereof; or a pharmaceutically acceptable salt, solvate, or prodrug thereof.

Another embodiment of the disclosure is a method of using the compounds of formula (I) or (II) as standards or controls in in vitro or in vivo assays in determining the efficacy of test compounds in modulating voltage-dependent sodium channels.

Specific embodiments of the compounds of the disclosure are described in more detail below in the Compound Preparation section.

### Utility and Testing of the Compounds of the Disclosure

In an embodiment, the present disclosure is directed to compounds of formula (I) or (II), as individual stereoisomers, enantiomers, or tautomers thereof or mixtures thereof; or pharmaceutically acceptable salts, solvates, or prodrugs thereof, which are useful in treating seizure disorders, for example, epilepsy and/or epileptic seizure disorders, in a mammal, preferably a human.

In another embodiment, compounds of formula (I) or (II), as individual stereoisomers, enantiomers, or tautomers thereof or mixtures thereof; or pharmaceutically acceptable salts, solvates, or prodrugs thereof, disclosed herein are useful in treating epilepsy, seizure disorders, partial seizures (such as simple, complex, secondary generalized, and focal onset), generalized seizures (such as absence, myoclonic, atonic, tonic and tonic clonic), and disorders including photosensitive epilepsy, self-induced syncope, intractable epilepsy, Angelman syndrome, benign rolandic epilepsy, CDKL5 disorder, childhood and juvenile absence epilepsy, Dravet syndrome, frontal lobe epilepsy, Glut1 deficiency syndrome, hypothalamic hamartoma, infantile spasms/West's syndrome, juvenile myoclonic epilepsy, Landau-Kleffner syndrome, Lennox-Gastaut syndrome (LGS), epilepsy with myoclonic-absences, Ohtahara syndrome, Panayiotopoulos syndrome, PCDH19 epilepsy, progressive myoclonic epilepsies, Rasmussen's syndrome, ring chromosome 20 syndrome, reflex epilepsies, temporal lobe epilepsy, Lafora progressive myoclonus epilepsy, neurocutaneous syndromes, tuberous sclerosis complex, early infantile epileptic encephalopathy, early onset epileptic encephalopathy, generalized epilepsy with febrile seizures plus (GEFS+), Rett syndrome, multiple sclerosis, Schizophrenia, autism, ataxia, hypotonia, paroxysmal dyskinesia, Alzheimer's disease and Tauopathies, including but not limited to Alzheimer's disease, Pick's disease, progressive supranuclear palsy, corticobasal syndrome, frontotemporal dementias, Argyrophilic grain disease, frontotemporal lobar degeneration, globular glial tauopathies, MAPT mutation, primary age-related tauopathy, neurofibrillary tangle dementia, chronic traumatic encephalopathy (CTE), aging-related tau astrogliopathy, Richardson syndrome, Down Syndrome, parkinsonism, pure akinesia with gait freezing, motor neuron symptoms or cerebellar ataxia, posttraumatic stress disorders (PTSD), or any combination of the these.

The present disclosure readily affords many different means for identification of sodium channel modulating agents that are useful as therapeutic agents. Identification of modulators of sodium channels can be assessed using a variety of in vitro and in vivo assays, e.g., measuring current, measuring membrane potential, measuring ion flux, (e.g., sodium), measuring sodium concentration, measuring second messengers and transcription levels, measuring neurotransmitter levels and using voltage-sensitive dyes, radioactive tracers, multi-electrode-arrays and patch-clamp electrophysiology.

One such protocol involves the screening of chemical agents for ability to modulate the activity of a sodium channel thereby identifying it as a modulating agent.

A typical assay described in (Crestey, F. et al., ACS Chem Neurosci (2015), Vol. 6, pp. 1302-1308), AA43279 (Frederiksen, K. et al., Eur J Neurosci (2017), Vol. 46, pp. 1887-1896) and Lu AE98134 (von Schoubyea, N. L. et al., Neurosci Lett (2018), Vol. 662, pp. 29-35) employs the use of automated planar patch clamp techniques to study the effects of the chemical agent on the gating of sodium channels. The sodium channel isoforms of interest are stably expressed in Human Embryonic Kidney Cells and the currents that flow through those channels in response to a depolarizing voltage clamp step from −120 mV to 0 mV are measured in the presence of increasing concentrations of the chemical agents. The area under the sodium current trace which correlates to the magnitude of sodium flux through the cell membrane is used to quantify the effects on gating of the channels. Other parameters that are measured in the assay include the peak current, time constant of open state inactivation and the voltage dependence of steady state inactivation properties. The concentration responses are used to determine potency of each chemical agents effects on modulating the sodium channel isoform gating. Such techniques are known to those skilled in the art, and may be developed, using current technologies, into low or medium throughput assays for evaluating compounds for their ability to modulate sodium channel behaviour.

The results of these assays provide the basis for analysis of the structure-activity relationship (SAR) between compounds of the disclosure and the sodium channel. Certain substituents on the core structure of a compound of the disclosure tend to provide more potent inhibitory or potentiating compounds. SAR analysis is one of the tools those skilled in the art may now employ to identify preferred embodiments of the compounds of the disclosure for use as therapeutic agents.

In an alternative use of the disclosure, the compounds of the disclosure can be used in in vitro or in vivo studies as exemplary agents for comparative purposes to find other compounds also useful in treatment of, or protection from, the various diseases disclosed herein.

In another embodiment, the compounds of formula (I) or (II), as individual stereoisomers, enantiomers, or tautomers thereof or mixtures thereof; or pharmaceutically acceptable salts, solvates, or prodrugs thereof, as set forth above in the Brief Summary, as stereoisomers, enantiomers, tautomers thereof or mixtures thereof, or pharmaceutically acceptable salts, solvates, or prodrugs thereof, and/or the pharmaceutical compositions described herein which comprise a pharmaceutically acceptable excipient and one or more compounds of the disclosure, as set forth above in the Brief Summary, as a stereoisomer, enantiomer, or tautomer thereof or mixtures thereof, or a pharmaceutically acceptable salt, solvate, or prodrug thereof, can be used in the preparation of a medicament for the treatment of a sodium channel-mediated disease or condition in a mammal.

### Pharmaceutical Compositions and Administration

This disclosure is also directed to pharmaceutical compositions containing the compounds of formula (I) or (II), as described above in the Brief Summary, as stereoisomers, enantiomers, or tautomers thereof or mixtures thereof; or pharmaceutically acceptable salts, solvates, or prodrugs thereof. In one embodiment, the present disclosure relates to a pharmaceutical composition comprising compounds of formula (I) or (II), as described above in the Brief Summary, as stereoisomers, enantiomers, or tautomers thereof or mixtures thereof; or pharmaceutically acceptable salts, solvates, or prodrugs thereof, in a pharmaceutically acceptable carrier, excipient or diluent and in an amount effective to modulate, preferably inhibit, voltage-gated sodium channels to treat certain diseases or conditions, such as epilepsy, when administered to an animal, preferably a mammal, most preferably a human patient.

Administration of the compounds of formula (I) or (II), as described above in the Brief Summary, as stereoisomers, enantiomers, or tautomers thereof or mixtures thereof; or pharmaceutically acceptable salts, solvates, or prodrugs thereof, in pure form or in an appropriate pharmaceutical composition, can be carried out via any of the accepted modes of administration of agents for serving similar utilities. The pharmaceutical compositions of the disclosure can be prepared by combining a compound of the disclosure with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, rectal, vaginal, and intranasal. The term “parenteral” as used herein includes subcutaneous injections, intravenous, intramuscular, intrathecal, intrasternal injection or infusion techniques. Pharmaceutical compositions of the disclosure are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the disclosure in aerosol form may hold a plurality of dosage units. Actual methods of preparing such dosage forms are known, or will be apparent, to those skilled in this art; for example, see The Science and Practice of Pharmacy, 20th Edition (Philadelphia College of Pharmacy and Science, 2000). The composition to be administered will, in any event, contain a therapeutically effective amount of a compound of the disclosure, or a pharmaceutically acceptable salt thereof, for treatment of a disease or condition of interest in accordance with the teachings of this disclosure.

The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids, such as water, saline, glycerol and ethanol, and the like. A thorough discussion of pharmaceutically acceptable carriers, diluents, and other excipients is presented in REMINGTON'S PHARMACEUTICAL SCIENCES (Mack Pub. Co., N.J. current edition).

A pharmaceutical composition of the disclosure may be in the form of a solid or liquid. In one aspect, the carrier(s) are particulate, so that the compositions are, for example, in tablet or powder form. The carrier(s) may be liquid, with the compositions being, for example, an oral syrup, injectable liquid, or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the disclosure, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the disclosure intended for either parenteral or oral administration should contain an amount of a compound of the disclosure such that a suitable dosage will be obtained. Typically, this amount is at least 0.01% of a compound of the disclosure in the composition. When intended for oral administration, this amount may be varied to be between 0.1 and about 70% of the weight of the composition. Preferred oral pharmaceutical compositions contain between about 4% and about 50% of the compound of the disclosure. Preferred pharmaceutical compositions and preparations according to the present disclosure are prepared so that a parenteral dosage unit contains between 0.01 to 10% by weight of the compound prior to dilution of the disclosure.

The pharmaceutical composition of the disclosure may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the disclosure from about 0.1 to about 10% w/v (weight per unit volume).

The pharmaceutical composition of the disclosure may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the disclosure may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the disclosure in solid or liquid form may include an agent that binds to the compound of the disclosure and thereby assists in the delivery of the compound. Suitable agents that may act in this capacity include a monoclonal or polyclonal antibody, a protein or a liposome.

The pharmaceutical composition of the disclosure may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the disclosure may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the disclosure may be prepared by methodology well known in the pharmaceutical art. For example, a pharmaceutical composition intended to be administered by injection can be prepared by combining a compound of the disclosure with sterile, distilled water so as to form a solution. A surfactant may be added to facilitate the formation of a homogeneous solution or suspension. Surfactants are compounds that non-covalently interact with the compound of the disclosure so as to facilitate dissolution or homogeneous suspension of the compound in the aqueous delivery system.

The compounds of the disclosure, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 Kg mammal) from about 0.001 mg/Kg (i.e., 0.07 mg) to about 100 mg/Kg (i.e., 7.0 g); preferably a therapeutically effective dose is (for a 70 Kg mammal) from about 0.01 mg/Kg (i.e., 0.7 mg) to about 50 mg/Kg (i.e., 3.5 g); more preferably a therapeutically effective dose is (for a 70 Kg mammal) from about 1 mg/kg (i.e., 70 mg) to about 25 mg/Kg (i.e., 1.75 g).

The ranges of effective doses provided herein are not intended to be limiting and represent preferred dose ranges. However, the most preferred dosage will be tailored to the individual subject, as is understood and determinable by one skilled in the relevant arts. (see, e.g., Berkow et al., eds., The Merck Manual, 16^{th} edition, Merck and Co., Rahway, N.J., 1992; Goodmanetna., eds., Goodman and Cilman's The Pharmacological Basis of Therapeutics, 10^{th} edition, Pergamon Press, Inc., Elmsford, N.Y., (2001); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985); Osolci al., eds., Remington's Pharmaceutical Sciences, 18^{th} edition, Mack Publishing Co., Easton, Pa. (1990); Katzung, Basic and Clinical Pharmacology, Appleton and Lange, Norwalk, Conn. (1992)).

The total dose required for each treatment can be administered by multiple doses or in a single dose over the course of the day, if desired. Generally, treatment is initiated with smaller dosages, which are less than the optimum dose of the compound. Thereafter, the dosage is increased by small increments until the optimum effect under the circumstances is reached. The diagnostic pharmaceutical compound or composition can be administered alone or in conjunction with other diagnostics and/or pharmaceuticals directed to the pathology, or directed to other symptoms of the pathology. The recipients of administration of compounds and/or compositions of the disclosure can be any vertebrate animal, such as mammals. Among mammals, the preferred recipients are mammals of the Orders Primate (including humans, apes and monkeys), Arteriodactyla (including horses, goats, cows, sheep, pigs), Rodenta (including mice, rats, rabbits, and hamsters), and Carnivora (including cats, and dogs). Among birds, the preferred recipients are turkeys, chickens and other members of the same order. The most preferred recipients are humans.

For topical applications, it is preferred to administer an effective amount of a pharmaceutical composition according to the disclosure to target area, e.g., skin surfaces, mucous membranes, and the like, which are adjacent to peripheral neurons which are to be treated. This amount will generally range from about 0.0001 mg to about 1 g of a compound of the disclosure per application, depending upon the area to be treated, whether the use is diagnostic, prophylactic or therapeutic, the severity of the symptoms, and the nature of the topical vehicle employed. A preferred topical preparation is an ointment, wherein about 0.001 to about 50 mg of active ingredient is used per cc of ointment base. The pharmaceutical composition can be formulated as transdermal compositions or transdermal delivery devices (“patches”). Such compositions include, for example, a backing, active compound reservoir, a control membrane, liner and contact adhesive. Such transdermal patches may be used to provide continuous pulsatile, or on demand delivery of the compounds of the present disclosure as desired.

The compositions of the disclosure can be formulated so as to provide quick, sustained or delayed release of the active ingredient after administration to the patient by employing procedures known in the art. Controlled release drug delivery systems include osmotic pump systems and dissolutional systems containing polymer-coated reservoirs or drug-polymer matrix formulations. Examples of controlled release systems are given in U.S. Pat. Nos. 3,845,770 and 4,326,525 and in P. J. Kuzma et al., Regional Anesthesia 22 (6): 543-551 (1997), all of which are incorporated herein by reference.

The compositions of the disclosure can also be delivered through intra-nasal drug delivery systems for local, systemic, and nose-to-brain medical therapies. Controlled Particle Dispersion (CPD)™ technology, traditional nasal spray bottles, inhalers or nebulizers are known by those skilled in the art to provide effective local and systemic delivery of drugs by targeting the olfactory region and paranasal sinuses.

The disclosure also relates to an intravaginal shell or core drug delivery device suitable for administration to the human or animal female. The device may be comprised of the active pharmaceutical ingredient in a polymer matrix, surrounded by a sheath, and capable of releasing the compound in a substantially zero order pattern on a daily basis similar to devises used to apply testosterone as described in PCT Published Patent Application No. WO 98/50016.

Current methods for ocular delivery include topical administration (eye drops), subconjunctival injections, periocular injections, intravitreal injections, surgical implants and iontophoresis (uses a small electrical current to transport ionized drugs into and through body tissues). Those skilled in the art would combine the best suited excipients with the compound for safe and effective intra-occular administration.

The most suitable route will depend on the nature and severity of the condition being treated. Those skilled in the art are also familiar with determining administration methods (e.g., oral, intravenous, inhalation, sub-cutaneous, rectal etc.), dosage forms, suitable pharmaceutical excipients and other matters relevant to the delivery of the compounds to a subject in need thereof.

### Combination Therapy

The compounds of the disclosure may be usefully combined with one or more other compounds of the disclosure or one or more other therapeutic agent or as any combination thereof, in the treatment of sodium channel-mediated diseases and conditions. For example, a compound of this disclosure may be administered simultaneously, sequentially, or separately in combination with other therapeutic agents, including, but not limited to:

Acetazolamide (Diamox), Brivaracetam (Briviact), Cannabidiol (Epidiolex), Carbamazepine (Tegretol), Cenobamate (Xcopri), Clobazam (Frisium), Clonazepam (Klonopin), Eslicarbazepine acetate (Aptiom, Zebinix), Ethosuximide (Zarontin), Felbamate (Felbatol), Fenfluramine (Fintepla), Gabapentin (Neurontin), Lacosamide (Vimpat), Lamotrigine (Lamictal), Levetiracetam (Keppra), Oxcarbazepine (Trileptal), Perampanel (Fycompa), Phenobarbital (Luminal), Phenytoin (Dilantin), Pregabalin (Lyrica), Primidone, Retigabine (Ezogabine), Rufinamide (Banzel), Stiripentol (Diacomit), Sulthiame, Tiagabine (Gabitril), Topiramate (Topamax), Valproate (Depakote), Vigabatrin (Sabril), Zonisamide (Zonegran).

As used herein “combination” refers to any mixture or permutation of one or more compounds of the disclosure and one or more other compounds of the disclosure or one

or more additional therapeutic agent. Unless the context makes clear otherwise, “combination” may include simultaneous or sequentially delivery of a compound of the disclosure with one or more therapeutic agents. Unless the context makes clear otherwise, “combination” may include dosage forms of a compound of the disclosure with another therapeutic agent. Unless the context makes clear otherwise, “combination” may include routes of administration of a compound of the disclosure with another therapeutic agent. Unless the context makes clear otherwise, “combination” may include formulations of a compound of the disclosure with another therapeutic agent. Dosage forms, routes of administration and pharmaceutical compositions include, but are not limited to, those described herein.

### Kits-of-Parts

The present disclosure also provides kits that contain a pharmaceutical composition which includes one or more compounds of the disclosure. The kit also includes instructions for the use of the pharmaceutical composition for modulating the activity of sodium channels, for the treatment of a seizure disorder, such as epilepsy, as well as other utilities as disclosed herein. Preferably, a commercial package will contain one or more unit doses of the pharmaceutical composition. For example, such a unit dose may be an amount sufficient for the preparation of an intravenous injection. It will be evident to those of ordinary skill in the art that compounds which are light and/or air sensitive may require special packaging and/or formulation. For example, packaging may be used which is opaque to light, and/or sealed from contact with ambient air, and/or formulated with suitable coatings or excipients.

### Compound Preparation

The following Reaction Schemes illustrate methods to make compounds of the disclosure, i.e., compounds of formula (I) or (II), as described above in the Brief Summary, as stereoisomers, enantiomers, or tautomers thereof or mixtures thereof; or pharmaceutically acceptable salts, solvates, or prodrugs thereof.

It is also understood that one skilled in the art would be able to make the compounds of the disclosure by similar methods or by methods known to one skilled in the art. It is also understood that one skilled in the art would be able to make in a similar manner as described below other compounds of the disclosure not specifically illustrated below by using the appropriate starting components and modifying the parameters of the synthesis as needed. In general, starting components may be obtained from sources such as Sigma Aldrich, Alfa Aesar, Combi-Blocks, Oakwood Chemicals, Matrix Scientific, and TCI, etc. or synthesized according to sources known to those skilled in the art (see, e.g., M. B. Smith and J. March, Advanced Organic Chemistry: Reactions, Mechanisms, and Structure, 6th edition (Wiley, 2007)) or prepared as described herein.

It is also understood that in the following description, combinations of substituents and/or variables of the depicted formulae are permissible only if such contributions result in stable compounds.

It will also be appreciated by those skilled in the art that in the process described below the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (e.g., t-butyldimethylsilyl, t-butyldiphenylsilyl or trimethylsilyl), tetrahydropyranyl, benzyl, and the like. Suitable protecting groups for amino, include t-butoxycarbonyl, benzyloxycarbonyl, p-methoxybenzyl, trityl and the like.

Protecting groups may be added or removed in accordance with standard techniques, which are known to one skilled in the art and as described herein.

The use of protecting groups is described in detail in Greene, T. W. and P. G. M. Wuts, Greene's Protective Groups in Organic Synthesis (2006), 4^{th} Ed., Wiley. The protecting group may also be a polymer resin such as a Wang resin or a 2-chlorotrityl-chloride resin.

It will also be appreciated by those skilled in the art, although such protected derivatives of compounds of this disclosure may not possess pharmacological activity as such, they may be administered to a mammal and thereafter metabolized in the body to form compounds of the disclosure which are pharmacologically active. Such derivatives may therefore be described as “prodrugs”. All prodrugs of compounds of formula (I) or (II) are included within the scope of the disclosure.

The compounds of formula (I) or (II) may contain at least one asymmetric carbon atom and thus can exist as racemates, enantiomers, and/or diastereoisomers. Specific enantiomers, or diastereoisomers may be prepared by utilizing the appropriate chiral starting material or through the use of suitable asymmetric synthetic methods. Alternatively, diastereoisomeric mixtures or racemic mixtures of compounds of formula (I) or (II) may be resolved into their respective enantiomers or diastereoisomers.

Methods for resolution of diastereoisomeric mixtures or racemic mixtures of the compounds of formula (I) or (II), as described herein, or intermediates prepared herein, are well known in the art (e.g., E. L. Eliel and S. H. Wilen, in Stereochemistry of Organic Compounds; John Wiley & Sons: New York, 1994; Chapter 7, and references cited therein). Suitable processes such as crystallization (e.g., preferential crystallization, preferential crystallization in the presence of additives), asymmetric transformation of racemates, chemical separation (e.g., formation and separation of diastereomers such as diastereomeric salt mixtures or the use of other resolving agents; separation via complexes and inclusion compounds), kinetic resolution (e.g., with titanium tartrate catalyst), enzymatic resolution (e.g., lipase mediated) and chromatographic separation (e.g., HPLC with chiral stationary phase and/or with simulated moving bed technology, or supercritical fluid chromatography and related techniques) are some of the examples that may be applied (see e.g., T. J. Ward, Analytical Chemistry, 2002, 2863-2872).

In general, compounds of formula (I) or (II), as described above in the Brief Summary, can be synthesized following the general procedure described below in Reaction Schemes 1-13 wherein appropriate starting materials, reagents, substituent groups, coupling partners, protecting groups, etc. are selected to carry out the reactions as shown to arrive at desired compound of formula (I) or (II).

For example, in some embodiments, of Reaction Schemes 1-13, R¹ represents

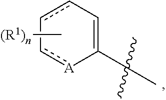

R² represents

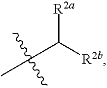

PG is a protecting group (e.g., an amine protecting group such as Boc or Fmoc), Z¹ is a halogen (e.g., F, Cl, Br, or I), Z² is a halogen (e.g., F, Cl, Br, or I), Z³ is a suitable coupling partner to Z¹ (e.g., a boronic acid or ester), Z⁴ is a suitable coupling partner to Z² (e.g., a carboxylic acid, boronic acid or ester), Z⁵ is a halogen (e.g., F, Cl, Br, or I), Z⁶ is a suitable coupling partner to Z⁵ (e.g., a dialkyl amine), Z⁷ is a halogen (e.g., F, Cl, Br, or I), and X is oxygen or carbon. X¹ is a suitable coupling group (i.e., that is capable of reacting with Z⁵) that forms a linker. In some embodiments, —X¹—R^{8b′} is R^{8b}. Other variable groups (e.g., R³) are as defined throughout the disclosure.

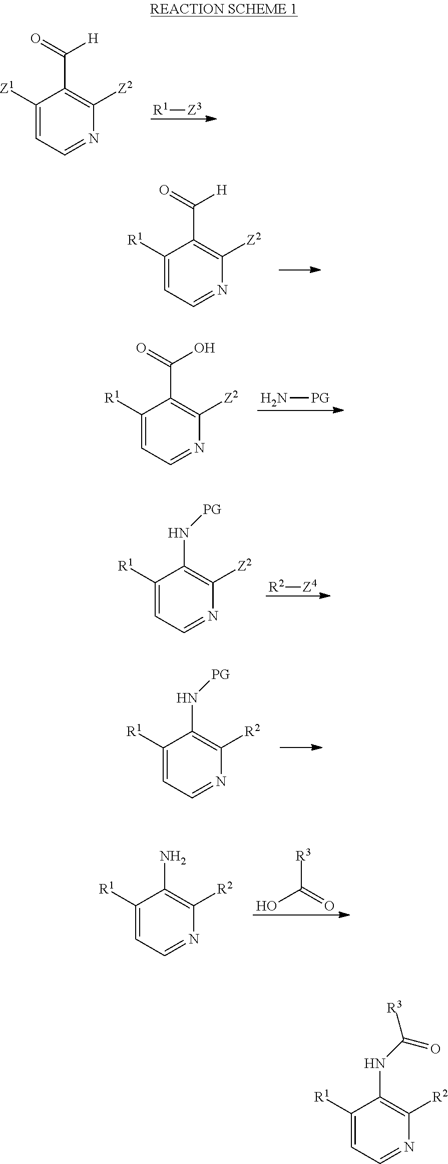

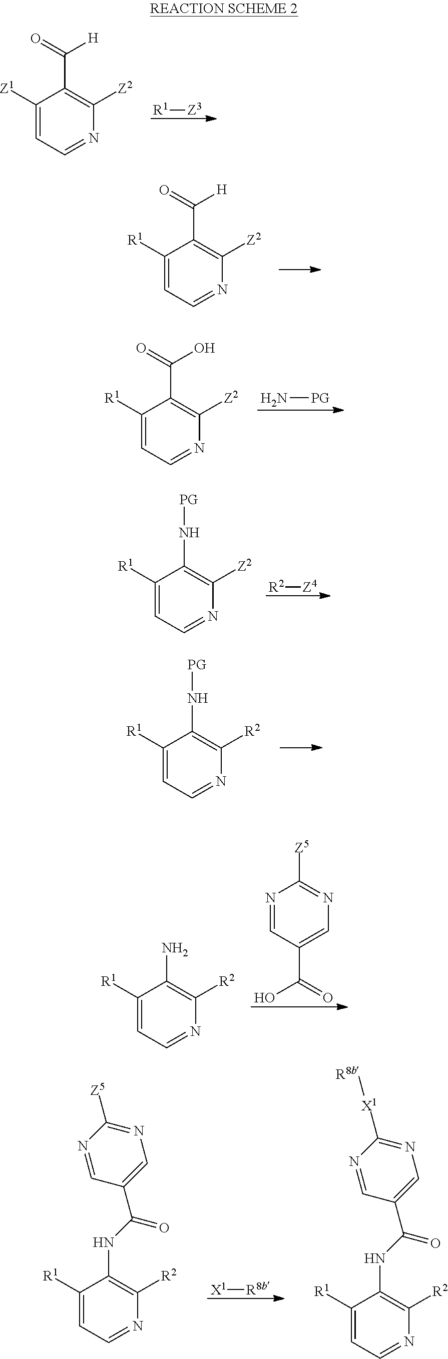

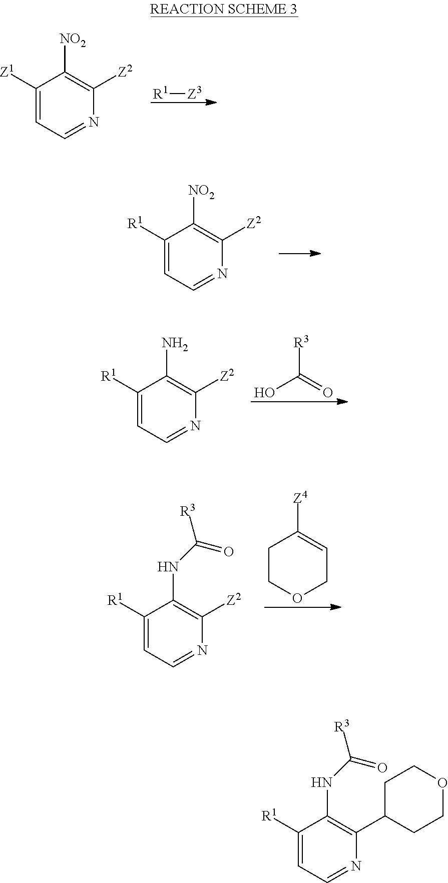

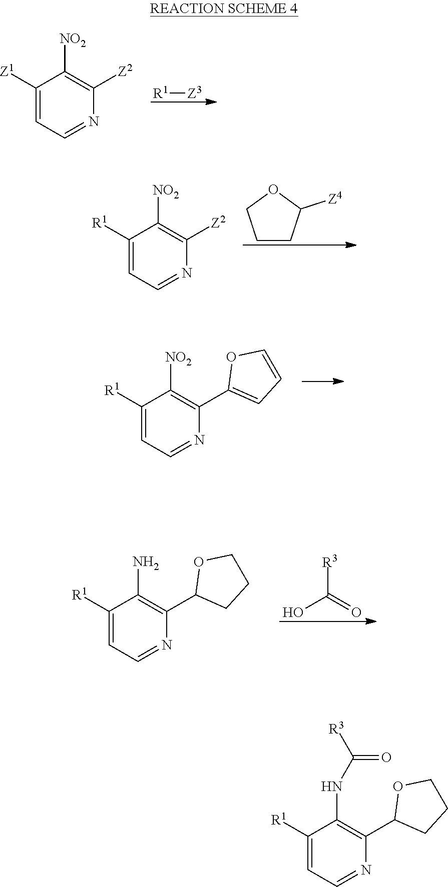

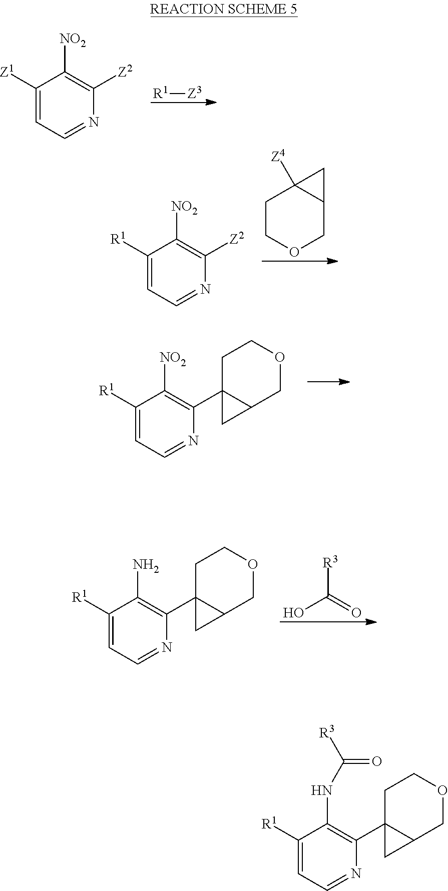

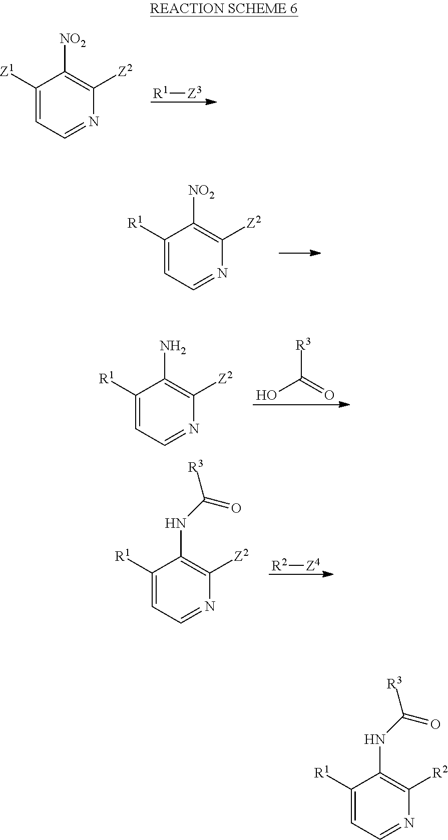

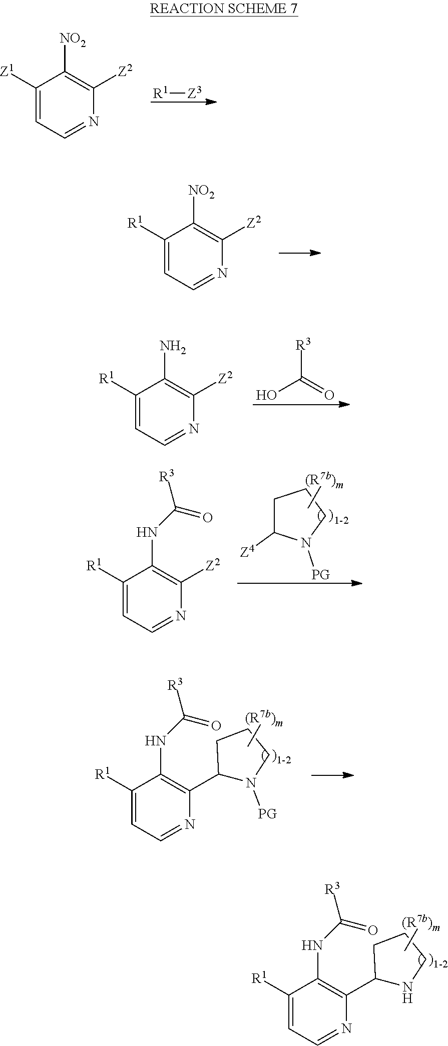

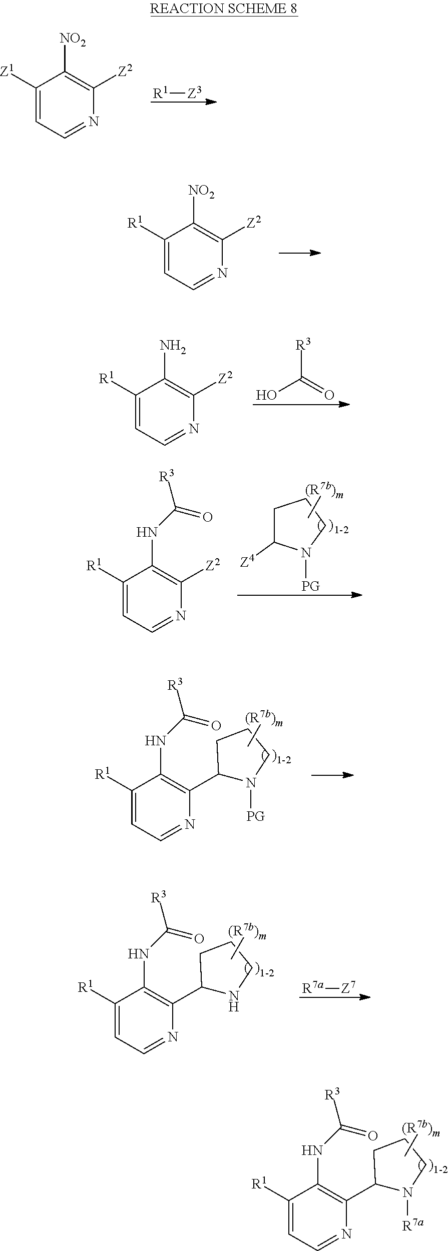

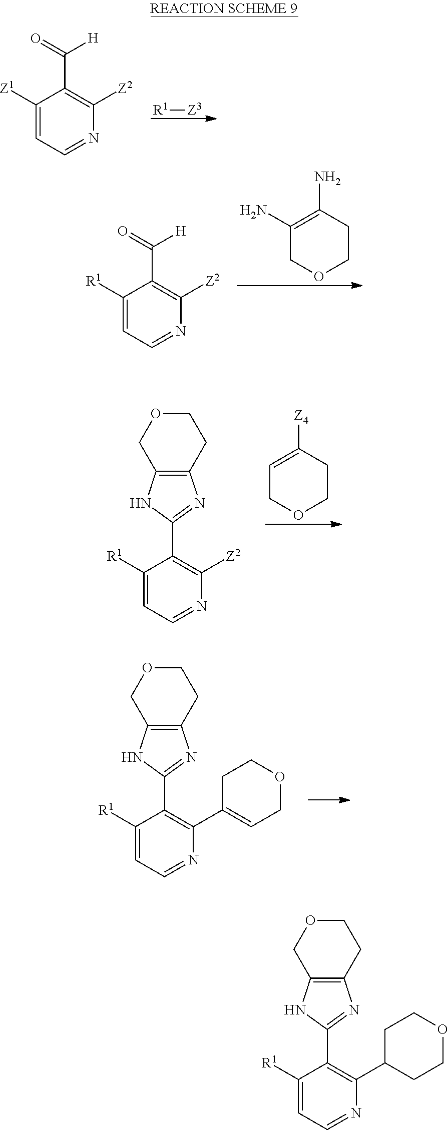

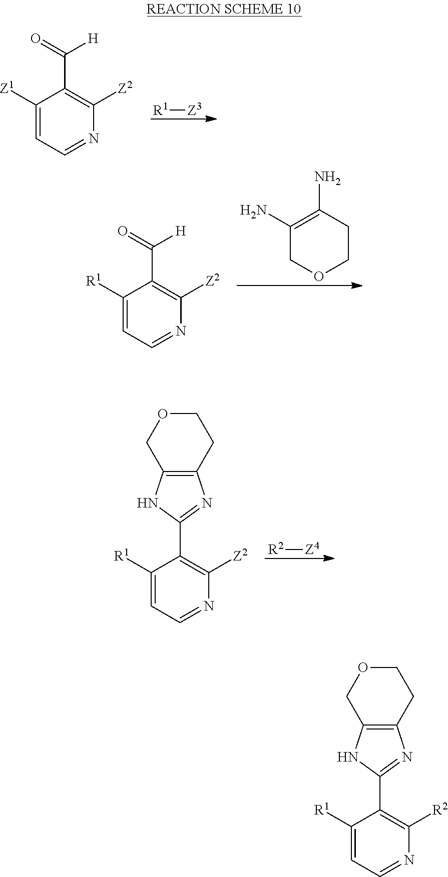

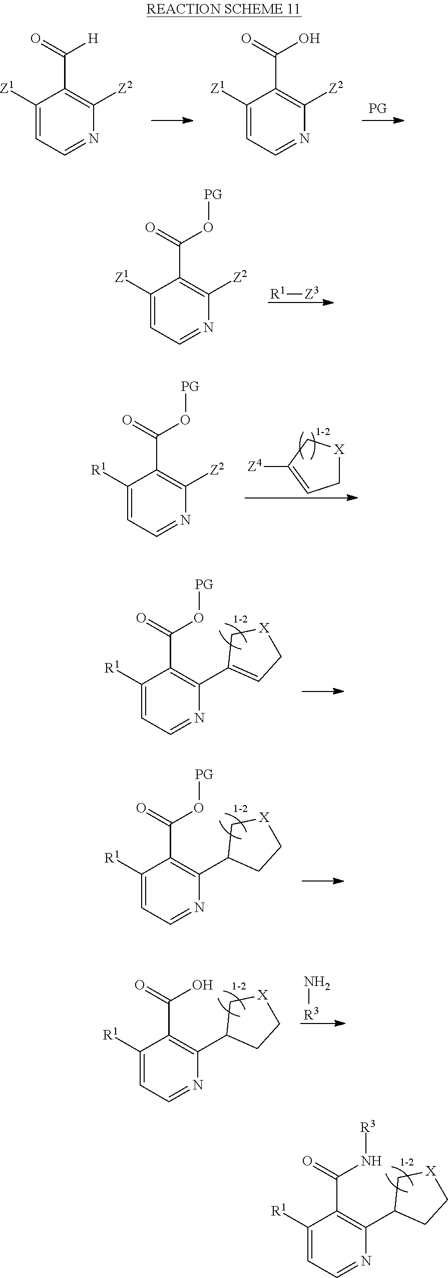

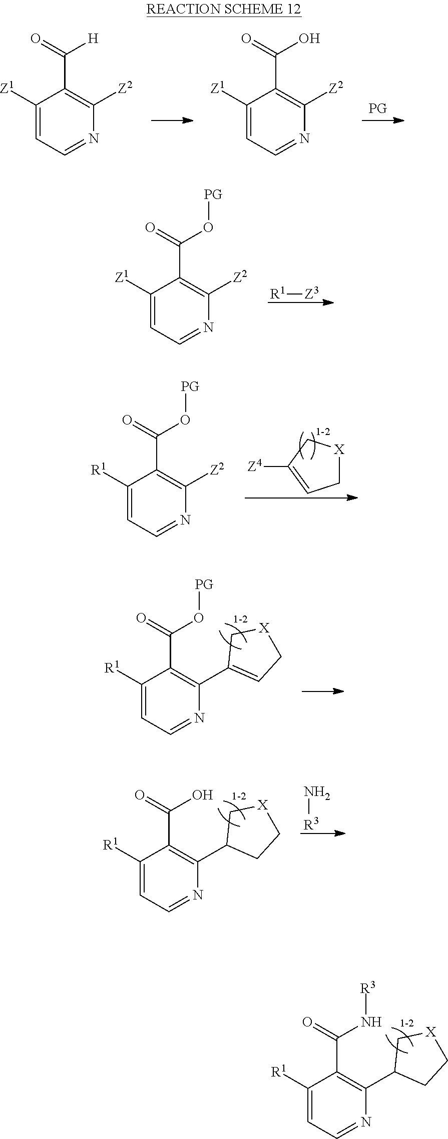

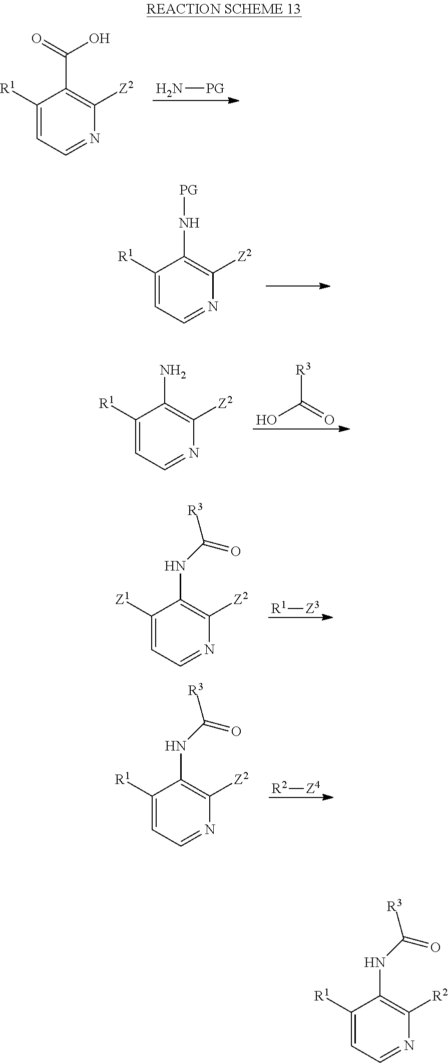

All of the compounds described below as being prepared which may exist in free base or acid form may be converted to their pharmaceutically acceptable salts by treatment with the appropriate inorganic or organic base or acid. Salts of the compounds prepared below may be converted to their free base or acid form by standard techniques.

Furthermore, all compounds of the disclosure which contain an acid or an ester group can be converted to the corresponding ester or acid, respectively, by methods known to one skilled in the art or by methods described herein.

The present disclosure also relates to novel intermediate compounds as defined above, all salts, solvates, and complexes thereof and all solvates and complexes of salts thereof as defined hereinbefore for compounds of formula (I) or (II). The disclosure includes all polymorphs of the aforementioned species and crystal habits thereof.

Embodiments disclosed herein are also meant to encompass all compounds being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶CI, ¹²³I, and ¹²⁵I, respectively.

Isotopically-labeled compounds can generally be prepared by conventional techniques known to those skilled in the art or by processes analogous to those described below and in the following Examples using an appropriate isotopically-labeled reagent in place of the non-labeled reagent previously employed.

The following Examples, which are directed to the synthesis of the compounds of the disclosure; and the following Biological Examples are provided as a guide to assist in the practice of the disclosure, and are not intended as a limitation on the scope of the disclosure.

In the Preparations and Examples below, unless otherwise indicated all temperatures are set forth in degrees Celsius. Commercially available reagents were purchased from suppliers such as Sigma Aldrich, Alfa Aesar, Combi-Blocks, Oakwood Chemicals, Matrix Scientific, and TCI, etc. and were used without further purification unless otherwise indicated. The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried. Yields were not optimized. Melting points were determined on a Buchi hot-stage apparatus and are uncorrected. ¹H NMR, ¹⁹F and ¹³C NMR data were obtained in deuterated CDCl₃, DMSO-d₆, CD₃OD, CD₃CN, or acetone-d₆ solvent solutions with chemical shifts (b) reported in parts-per-million (ppm) relative to trimethylsilane (TMS) or the residual non-deuterated solvent peaks as the reference standard. Data are reported as follows, if applicable: chemical shift, multiplicity, coupling constant in Hz, and number of protons, fluorine or carbon atoms. When peak multiplicities are reported, the following abbreviates are used: s (singlet), d (doublet), t (triplet), q (quartet), m (multiplet), br (broadened), dd (doublet of doublets), dt (doublet of triplets). Coupling constants, when given, are reported in Hz (Hertz).

### Example 1

### Synthesis of 1-cyclobutyl-N-[2-(4,4-difluorocyclohexyl)-4-(2-fluorophenyl)-3-pyridyl]pyrazole-4-carboxamide

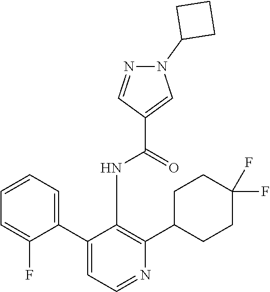 [SMILES: O=C(Nc1c(-c2ccccc2F)ccnc1C1CCC(F)(F)CC1)c1cnn(C2CCC2)c1]

### Step 1. Preparation of 2-chloro-4-(2-fluorophenyl)nicotinaldehyde

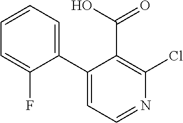 [SMILES: O=C(O)c1c(-c2ccccc2F)ccnc1Cl]

A mixture of 2-chloro-4-iodonicotinaldehyde (6.95 g, 26.0 mmol), 1,4-dioxane (86 mL), and water (10 mL) was sparged with nitrogen for 10 min. The flask was charged with 2-fluorophenylboronic acid (4.0 g, 29 mmol), potassium carbonate (9.0 g, 65 mmol), and [1,1′-bis(diphenylphosphino)ferrocene]dichloropalladium(II), complex with dichloromethane (2.22 g, 2.60 mmol), and sparged for 2 min. The reaction mixture was stirred at 80° C. for 2 h. After cooling to ambient temperature, the reaction mixture was diluted with ethyl acetate (300 mL). The organic layer was washed with saturated ammonium chloride (2×100 mL) and brine (150 mL). The organic solution was dried over anhydrous magnesium sulfate, filtered, and concentrated in vacuo. Purification of the residue by column chromatography, eluting with 0-30% ethyl acetate in heptane, afforded the title compound as a colorless oil that solidified upon standing (5.82 g, 95% yield): MS (ESI+) m/z 235.0 (M+1), 237.0 (M+1).

### Step 2. Preparation of 2-chloro-4-(2-fluorophenyl)nicotinic acid

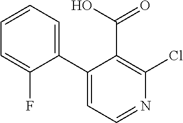 [SMILES: O=C(O)c1c(-c2ccccc2F)ccnc1Cl]

A mixture of 2-chloro-4-(2-fluorophenyl)nicotinaldehyde (5.0 g, 21 mmol), 1,4-dioxane (185 mL), and water (36 mL) was cooled in an ice/water bath. To this solution was added potassium permanganate (5.02 g, 31.8 mmol). The solution was warmed to ambient temperature and stirred for 4 h. The pH of the solution was adjusted to >12 with 5M sodium hydroxide solution (5 mL), and filtered. The pH of the filtrate was adjusted to <5 using concentrated hydrochloric acid (8 mL). The aqueous mixture was diluted with ethyl acetate (300 mL) and separated. The organic layer was washed with brine (2×100 mL). The organic solution was dried over magnesium sulfate, filtered, and concentrated in vacuo. The colorless oil was used without further purification (4.8 g, 90% yield): MS (ESI+) m/z 252.0 (M+1), 254.0 (M+1).

### Step 3. Preparation of tert-butyl N-[2-chloro-4-(2-fluorophenyl)-3-pyridyl]carbamate

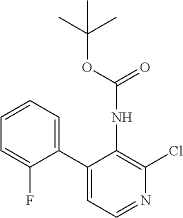 [SMILES: CC(C)(C)OC(=O)Nc1c(-c2ccccc2F)ccnc1Cl]

To a solution of 2-chloro-4-(2-fluorophenyl)nicotinic acid (1.20 g, 4.77 mmol) in tert-butanol (22.6 mL) and N,N-dimethylformamide (22.6 mL) was added diphenylphosphonic azide, and triethylamine (1.21 g, 11.9 mmol). The solution was heated at 95° C. for 2 h. The reaction mixture was cooled to ambient temperature and diluted with ethyl acetate (500 mL). The reaction mixture was washed with saturated sodium bicarbonate solution (3×100 mL), water (50 mL), and brine (50 mL). The organic layer was dried over anhydrous magnesium sulfate, filtered and concentrated in vacuo. Purification of the residue by column chromatography, eluting with 0-25% ethyl acetate in heptane, afforded the title compound as a colorless oil (1.27 g, 83% yield): MS (ES+) m/z 323.2 (M+1), 325.2 (M+1).

### Step 4. Preparation of tert-butyl N-[2-(4,4-difluorocyclohexyl)-4-(2-fluorophenyl)-3-pyridyl]carbamate

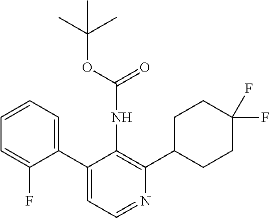 [SMILES: CC(C)(C)OC(=O)Nc1c(-c2ccccc2F)ccnc1C1CCC(F)(F)CC1]

To a vial containing tert-butyl N-[2-chloro-4-(2-fluorophenyl)-3-pyridyl]carbamate (1.07 g, 3.32 mmol) was added 4,4-difluorocyclohexanecarboxylic acid (0.93 g, 5.7 mmol), (4,4″-di-t-butyl-2,2″-bipyridine)bis[3,5-difluoro-2-[5-trifluoromethyl-2-pyridinyl-kN)phenyl-kC]iridium(II) hexafluorophosphate (0.037 g, 0.033 mmol), dichloro(dimethoxyethane)nickel (0.073 g, 0.33 mmol), 4-tert-butyl-2-(4-tert-butyl-2-pyridyl)pyridine (0.13 g, 0.50 mmol), cesium carbonate (1.95 g, 5.98 mmol), and N,N-dimethylformamide (55 mL). The vial was sealed and the reaction mixture was stirred in front of Kessil PR160L lights (440 nm) for 24 h. The reaction mixture was diluted with ethyl acetate (250 mL) and washed with saturated ammonium chloride solution (2×50 mL), water (50 mL), and brine (50 mL). The organic layer was dried over anhydrous magnesium sulfate, filtered and concentrated in vacuo. Purification of the residue by column chromatography, eluting with 0-20% ethyl acetate in heptane, afforded the title compound as a colorless solid (0.82 g, 61% yield): MS (ES+) m/z 407.2 (M+1).

### Step 5. Preparation of 2-(4,4-difluorocyclohexyl)-4-(2-fluorophenyl)pyridin-3-amine

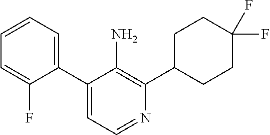 [SMILES: Nc1c(-c2ccccc2F)ccnc1C1CCC(F)(F)CC1]

To a mixture of tert-butyl N-[2-(4,4-difluorocyclohexyl)-4-(2-fluorophenyl)-3-pyridyl]carbamate (0.82 g, 2.5 mmol) in 1,4-dioxane (4.0 mL) was added 4 M hydrogen chloride in 1,4-dioxane (7.5 mL, 30 mmol). The reaction mixture was stirred for 18 h. The reaction mixture was diluted with ethyl acetate (90 mL) and washed with saturated ammonium hydroxide solution (2×20 mL), and brine (20 mL). The organic layer was dried over anhydrous magnesium sulfate, filtered and concentrated in vacuo. Purification of the residue by column chromatography, eluting with 0-40% ethyl acetate in heptane, afforded the title compound as a yellow solid (0.61 g, 98% yield): ¹H-NMR (400 MHz; CDCl₃) δ 8.10 (d, J=4.9 Hz, 1H), 7.26-7.07 (m, 4H), 6.93 (d, J=4.9 Hz, 1H), 3.71-3.65 (s, 2H), 2.82-2.77 (m, 1H), 2.35-2.27 (m, 2H), 2.18-2.08 (m, 2H), 2.04-1.98 (m, 2H), 1.96-1.82 (m, 2H); MS (ES+) m/z 307.2 (M+1).

### Step 6. Preparation of 1-cyclobutyl-N-[2-(4,4-difluorocyclohexyl)-4-(2-fluorophenyl)-3-pyridyl]pyrazole-4-carboxamide

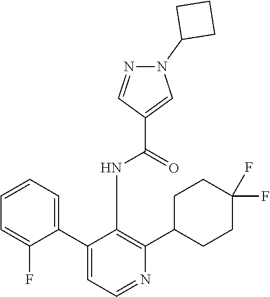 [SMILES: O=C(Nc1c(-c2ccccc2F)ccnc1C1CCC(F)(F)CC1)c1cnn(C2CCC2)c1]

To a mixture of 2-(4,4-difluorocyclohexyl)-4-(2-fluorophenyl)pyridin-3-amine (0.050 g, 0.16 mmol), 1-cyclobutyl-1H-pyrazole-4-carboxylic acid (0.041 g, 0.24 mmol) and 2-chloro-1-methylpyridinium iodide (0.10 g, 0.41 mmol) was added anhydrous tetrahydrofuran (3.3 mL). The solution was heated at 55° C. for 1 min before N-ethyl-N-isopropylpropan-2-amine (0.21 g, 1.6 mmol) was added. The reaction mixture was stirred at 55° C. for 18 h. The reaction mixture was cooled to ambient temperature and diluted with ethyl acetate (100 mL). The reaction mixture was washed with saturated ammonium chloride solution (2×50 mL). The organic layer was dried over anhydrous magnesium sulfate, filtered and concentrated in vacuo. Purification of the residue by column chromatography, eluting with 10-80% ethyl acetate in heptane, provided the title compound as a colorless solid (0.021 g, 28% yield): ¹H-NMR (400 MHz; DMSO-d₆) δ 9.56 (s, 1H), 8.56 (d, J=4.9 Hz, 1H), 8.21 (d, J=0.4 Hz, 1H), 7.87 (s, 1H), 7.41-7.23 (m, 4H), 7.18 (td, J=7.5, 1.1 Hz, 1H), 4.83 (quintet, J=8.4 Hz, 1H), 3.15-3.10 (m, 1H), 2.47-2.32 (m, 4H), 2.12-2.06 (m, 2H), 1.93-1.73 (m, 8H); MS (ES+) m/z 455.2 (M+1).

### Example 2

### Synthesis of N-[2-(4,4-difluorocyclohexyl)-4-(2-fluorophenyl)-3-pyridyl]-2-(dimethylamino)pyrimidine-5-carboxamide

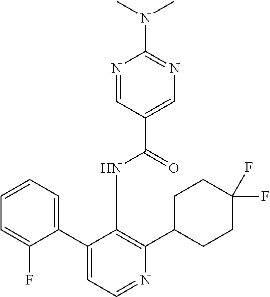 [SMILES: CN(C)c1ncc(C(=O)Nc2c(-c3ccccc3F)ccnc2C2CCC(F)(F)CC2)cn1]

### Step 1. Preparation of 2-chloro-N-[2-(4,4-difluorocyclohexyl)-4-(2-fluorophenyl)-3-pyridyl]pyrimidine-5-carboxamide

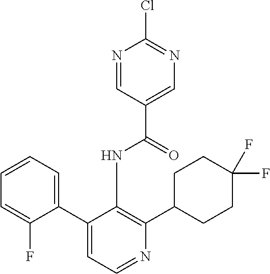 [SMILES: O=C(Nc1c(-c2ccccc2F)ccnc1C1CCC(F)(F)CC1)c1cnc(Cl)nc1]

To a mixture of 2-(4,4-difluorocyclohexyl)-4-(2-fluorophenyl)pyridin-3-amine (0.20 g, 0.65 mmol), 2-chloropyrimidine-5-carboxylic acid (0.16 g, 0.98 mmol) and 2-chloro-1-methylpyridinium iodide (0.42 g, 1.6 mmol) was added anhydrous tetrahydrofuran (13 mL). The solution was heated at 55° C. for 1 min before N-ethyl-N-isopropylpropan-2-amine (0.84 g, 6.5 mmol) was added. The reaction mixture was stirred at 55° C. for 45 h. The reaction mixture was cooled to ambient temperature and diluted with ethyl acetate (100 mL). The reaction mixture was washed with saturated ammonium chloride solution (2×50 mL). The organic layer was dried over anhydrous magnesium sulfate, filtered and concentrated in vacuo. Purification of the residue by column chromatography, eluting with 5-50% ethyl acetate in heptane, provided the title compound as a colorless solid (0.23 g, 80% yield): MS (ES+) m/z 447.2 (M+1), 447.2 (M+1).

### Step 2. Preparation of N-[2-(4,4-difluorocyclohexyl)-4-(2-fluorophenyl)-3-pyridyl]-2-(dimethylamino)pyrimidine-5-carboxamide

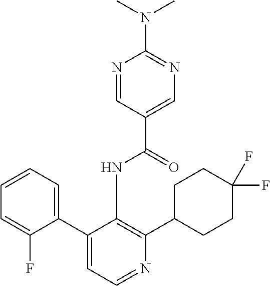 [SMILES: CN(C)c1ncc(C(=O)Nc2c(-c3ccccc3F)ccnc2C2CCC(F)(F)CC2)cn1]

To a mixture of 2-chloro-N-[2-(4,4-difluorocyclohexyl)-4-(2-fluorophenyl)-3-pyridyl]pyrimidine-5-carboxamide (0.080 g, 0.18 mmol) in anhydrous 1-methyl-2-pyrrolidinone (0.4 mL) was added dimethylamine hydrochloride (0.15 g, 1.8 mmol) and 60% sodium hydride dispersion in mineral oil (0.014 g, 0.36 mmol). The solution was stirred at ambient temperature for 1 h. The reaction mixture was diluted with ethyl acetate (100 mL) and washed with saturated ammonium chloride solution (2×50 mL). The organic layer was dried over anhydrous magnesium sulfate, filtered and concentrated in vacuo. Purification of the residue by column chromatography, eluting with 10-100% ethyl acetate in heptane, provided the title compound as a colorless solid (0.024 g, 29% yield): ¹H-NMR (400 MHz; DMSO-d₆) δ 9.82 (s, 1H), 8.63 (s, 2H), 8.58 (d, J=4.9 Hz, 1H), 7.41-7.31 (m, 3H), 7.29-7.24 (m, 1H), 7.20 (td, J=7.5, 1.1 Hz, 1H), 3.16 (d, J=6.3 Hz, 7H), 2.11-2.07 (m, 2H), 1.94-1.86 (m, 6H); MS (ES+) m/z 456.2 (M+1).

### Example 3

### Synthesis of N-[2-(4,4-difluorocyclohexyl)-4-(2-fluorophenyl)-3-pyridyl]-2-ethoxy-pyrimidine-5-carboxamide

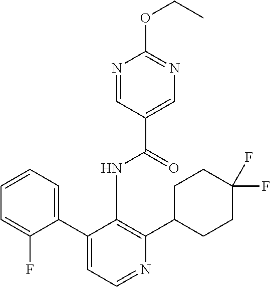 [SMILES: CCOc1ncc(C(=O)Nc2c(-c3ccccc3F)ccnc2C2CCC(F)(F)CC2)cn1]

To 2-chloro-N-[2-(4,4-difluorocyclohexyl)-4-(2-fluorophenyl)-3-pyridyl]pyrimidine-5-carboxamide (0.080 g, 0.18 mmol) was added anhydrous ethanol (0.6 mL) and 60% sodium hydride dispersion in mineral oil (0.014 g, 0.36 mmol). The solution was stirred at ambient temperature for 1 h. The reaction mixture was diluted with ethyl acetate (100 mL) and washed with saturated ammonium chloride solution (2×50 mL). The organic layer was dried over anhydrous magnesium sulfate, filtered and concentrated in vacuo. Purification of the residue by column chromatography, eluting with 10-100% ethyl acetate in heptane, provided the title compound as a colorless solid (0.018 g, 21% yield): ¹H-NMR (400 MHz; DMSO-d₆) δ 10.17 (s, 1H), 8.85-8.83 (m, 2H), 8.60 (d, J=4.9 Hz, 1H), 7.44-7.34 (m, 3H), 7.30-7.26 (m, 1H), 7.22 (td, J=7.5, 1.1 Hz, 1H), 4.42 (q, J=7.1 Hz, 2H), 3.20-3.15 (m, 1H), 2.13-2.06 (m, 2H), 1.98-1.83 (m, 6H), 1.35 (t, J=7.1 Hz, 3H); MS (ES+) m/z 457.0 (M+1).

### Example 4

### Synthesis of N-[2-(4,4-difluorocyclohexyl)-4-(2-fluorophenyl)-3-pyridyl]-2-isopropoxy-pyrimidine-5-carboxamide

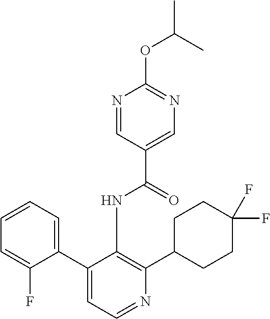 [SMILES: CC(C)Oc1ncc(C(=O)Nc2c(-c3ccccc3F)ccnc2C2CCC(F)(F)CC2)cn1]

To 2-chloro-N-[2-(4,4-difluorocyclohexyl)-4-(2-fluorophenyl)-3-pyridyl]pyrimidine-5-carboxamide (0.080 g, 0.18 mmol) was added anhydrous isopropanol (0.6 mL), anhydrous 1-methyl-2-pyrrolidinone (0.40 mL), and 60% sodium hydride dispersion in mineral oil (0.014 g, 0.36 mmol). The solution was stirred at 50° C. for 1 h. The reaction mixture was diluted with ethyl acetate (100 mL) and washed with saturated ammonium chloride solution (2×50 mL). The organic layer was dried over anhydrous magnesium sulfate, filtered and concentrated in vacuo. Purification of the residue by column chromatography, eluting with 10-100% ethyl acetate in heptane, provided the title compound as a colorless solid (0.020 g, 21% yield): ¹H-NMR (400 MHz; DMSO-d₆) δ 10.15 (d, J=6.7 Hz, 1H), 8.83 (d, J=3.7 Hz, 2H), 8.60 (d, J=4.9 Hz, 1H), 7.44-7.33 (m, 3H), 7.31-7.26 (m, 1H), 7.22 (td, J=7.5, 1.1 Hz, 1H), 5.26 (quintet, J=6.2 Hz, 1H), 3.19-3.15 (m, 1H), 2.12-2.06 (m, 2H), 2.02-1.85 (m, 6H), 1.34 (t, J=4.1 Hz, 6H); MS (ES+) m/z 471.4 (M+1).

### Example 5

### Synthesis of N-[2-(4,4-difluorocyclohexyl)-4-(2-fluorophenyl)-3-pyridyl]-4-methoxy-piperidine-1-carboxamide

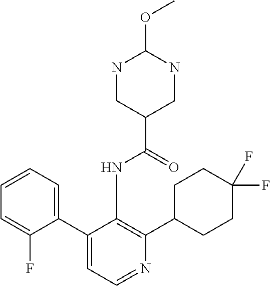 [SMILES: COC1NCC(C(=O)Nc2c(-c3ccccc3F)ccnc2C2CCC(F)(F)CC2)CN1]

To 2-(4,4-difluorocyclohexyl)-4-(2-fluorophenyl)pyridin-3-amine (0.10 g, 0.34 mmol) was added anhydrous tetrahydrofuran (1.1 mL) and the mixture was cooled in an ice-water bath. Solid triphosgene (0.058 g, 0.20 mmol) was added. The solution was stirred at 0° C. for 2 h then warmed to ambient temperature for 1 h. To the solution was added 4-methoxypiperidine (0.1 g, 1.0 mmol), anhydrous tetrahydrofuran (1.1 mL), and N-ethyl-N-isopropylpropan-2-amine (0.42 g, 3.3 mmol) was added. The reaction was stirred at ambient temperature for 12 h. The reaction mixture was diluted with ethyl acetate (100 mL), washed with saturated ammonium chloride (2×50 mL), dried over anhydrous magnesium sulfate, filtered, and concentrated in vacuo. Purification of the residue by column chromatography, eluting with a gradient of 10 to 100% of ethyl acetate in heptane, followed by trituration with diethyl ether (5 mL) and filtration provided the title compound as a colorless solid (0.036 g, 23% yield): ¹H-NMR (400 MHz; DMSO-d₆) δ 8.46 (d, J=4.9 Hz, 1H), 8.05 (s, 1H), 7.43 (dddd, J=8.5, 7.1, 5.4, 1.6 Hz, 1H), 7.34-7.20 (m, 4H), 3.61-3.58 (m, 2H), 3.33 (s, 3H), 3.23 (s, 3H), 3.13-3.10 (m, 1H), 2.95-2.89 (m, 2H), 2.15-2.09 (m, 2H), 1.90-1.78 (m, 6H), 1.65-1.61 (m, 2H), 1.16-1.07 (m, 2H); MS (ES+) m/z 448.2 (M+1).

### Example 6

### Synthesis of N-[2-(4,4-difluorocyclohexyl)-4-(2,5-difluorophenyl)-3-pyridyl]-2-isopropyl-pyrimidine-5-carboxamide

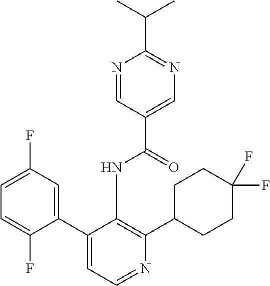 [SMILES: CC(C)c1ncc(C(=O)Nc2c(-c3cc(F)ccc3F)ccnc2C2CCC(F)(F)CC2)cn1]

### Step 1. Preparation of 2-chloro-4-(2,5-difluorophenyl)pyridine-3-carbaldehyde

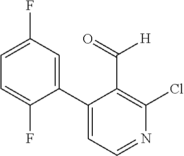 [SMILES: O=Cc1c(-c2cc(F)ccc2F)ccnc1Cl]

To 2-chloro-4-iodonicotinaldehyde (10.0 g, 37.4 mmol) was added 1,4-dioxane (135 mL) and water (15.0 mL) and the mixture was sparged with nitrogen for 10 min. To the mixture was added 2.5-difluorophenylboronc acid (6.49 g, 41.1 mmol), potassium carbonate (15.5 g, 112 mmol), and [1,1′-Bis(diphenylphosphino)ferrocene]dichloropalladium(II), complex with dichloromethane (3.17 g, 3.74 mmol), and the solution was sparged with nitrogen for 2 min. The flask was sealed under a nitrogen atmosphere and heated to 85° C. for 5 h. The reaction mixture was cooled to ambient temperature and diluted with ethyl acetate (300 mL). The organic layer was washed with saturated ammonium chloride (2×50 mL), dried over anhydrous magnesium sulfate, filtered and concentrated in vacuo. Purification of the residue by column chromatography, eluting with 0-25% ethyl acetate in heptane, afforded the title compound as a brown solid (9.4 g, 99% yield): MS (ES+) m/z 254.0 (M+1), 256.0 (M+1).

### Step 2. Preparation of 2-chloro-4-(2,5-difluorophenyl)pyridine-3-carboxylic acid

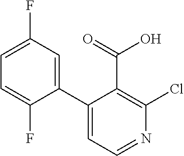 [SMILES: O=C(O)c1c(-c2cc(F)ccc2F)ccnc1Cl]

To 2-chloro-4-(2,5-difluorophenyl)pyridine-3-carbaldehyde (9.4 g, 37 mmol) was added tert-butanol (468 mL), dichloromethane (128 mL), and 2-methyl-2-butene (119 mL). The reaction was cooled to 0° C. before a mixture of sodium dihydrogenphosphate (15.7 g, 131 mmol) and sodium chlorite (10.6 g, 93.6 mmol) in water (205 mL) was added dropwise. The reaction was allowed to warm to ambient temperature and stirred for 18 h. The reaction mixture was diluted with ethyl acetate (200 mL) and washed with 1 M hydrochloric acid until the pH of the aqueous solution was <2. The aqueous layer was washed with ethyl acetate (3×500 mL), and the combined organic phases were washed with 1 M hydrochloric acid (2×100 mL) and brine (2×100 mL). The organic solution was dried over anhydrous magnesium sulfate, filtered, and concentrated in vacuo. The colorless oil was used without further purification (10.1 g, 100% yield): MS (ES+) m/z: 270.0 (M+1), 272.0 (M+1).

### Step 3. Preparation of tert-butyl N-[2-chloro-4-(2,5-difluorophenyl)-3-pyridyl]carbamate

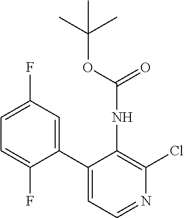 [SMILES: CC(C)(C)OC(=O)Nc1c(-c2cc(F)ccc2F)ccnc1Cl]

To 2-chloro-4-(2,5-difluorophenyl)pyridine-3-carboxylic acid (10.1 g, 37.5 mmol) was added tert-butanol (150 mL) and N,N-dimethylformamide (150 mL), diphenylphosphonic azide (15.5 g, 56.2 mmol), and triethylamine (9.48 g, 93.6 mmol). The solution was heated at 95° C. for 2 h. The reaction mixture was cooled to ambient temperature and diluted with ethyl acetate (600 mL). The reaction mixture was washed with saturated ammonium chloride (150 mL), saturated sodium bicarbonate (2×150 mL), water (150 mL), and brine (50 mL). The organic layer was dried over anhydrous magnesium sulfate, filtered and concentrated in vacuo. Purification of the residue by column chromatography, eluting with 0-50% ethyl acetate in heptane, afforded the title compound as a colorless solid (8.5 g, 67% yield): ¹H-NMR (400 MHz; DMSO-d₆) δ 8.34 (d, J=5.0 Hz, 1H), 7.43-7.38 (m, 1H), 7.30-7.25 (m, 1H), 7.17-7.07 (m, 2H), 6.32-6.30 (s, 1H), 1.31 (s, 9H); MS (ES+) m/z 341.2 (M+1), 343.2 (M+1).

### Step 4. Preparation of tert-butyl N-[2-(4,4-difluorocyclohexyl)-4-(2,5-difluorophenyl)-3-pyridyl]carbamate

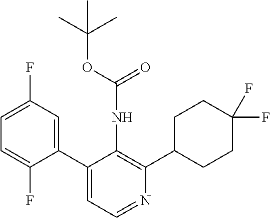 [SMILES: CC(C)(C)OC(=O)Nc1c(-c2cc(F)ccc2F)ccnc1C1CCC(F)(F)CC1]

To tert-butyl N-[2-chloro-4-(2,5-difluorophenyl)-3-pyridyl]carbamate (1.5 g, 4.6 mmol) was added 4,4-difluorocyclohexanecarboxylic acid (1.3 g, 7.9 mmol), (4,4″-Di-t-butyl-2,2″-bipyridine)bis[3,5-difluoro-2-[5-trifluoromethyl-2-pyridinyl-kN)phenyl-kC]iridium(III) hexafluorophosphate (0.052 g, 0.046 mmol), dichloro(dimethoxyethane)nickel (0.10 g, 0.46 mmol), 4-tert-butyl-2-(4-tert-butyl-2-pyridyl)pyridine (0.19 g, 0.70 mmol), cesium carbonate (2.7 g, 8.4 mmol), and N,N-dimethylformamide (77 mL). The vial was sealed and the reaction mixture was stirred in front of Kessil PR160L lights (440 nm) for 24 h. The reaction mixture was diluted with ethyl acetate (250 mL) and washed with saturated ammonium chloride solution (2×50 mL), water (50 mL), and brine (50 mL). The organic layer was dried over anhydrous magnesium sulfate, filtered and concentrated in vacuo. Purification of the residue by column chromatography, eluting with 0-40% ethyl acetate in heptane, afforded the title compound as a yellow solid (0.98 g, 52% yield): MS (ES+) m/z 425.4 (M+1).

### Step 5. Preparation of 2-(4,4-difluorocyclohexyl)-4-(2,5-difluorophenyl)pyridin-3-amine

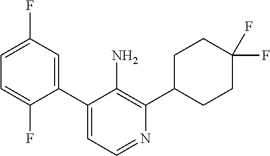 [SMILES: Nc1c(-c2cc(F)ccc2F)ccnc1C1CCC(F)(F)CC1]

To tert-butyl N-[2-(4,4-difluorocyclohexyl)-4-(2,5-difluorophenyl)-3-pyridyl]carbamate (0.98 g, 2.4 mmol) was added a 4 M solution of hydrochloric acid in 1,4-dioxane (10 mL). The reaction mixture was stirred for 18 h. The reaction mixture was diluted with ethyl acetate (100 mL), washed with saturated sodium bicarbonate (2×50 mL), dried over anhydrous magnesium sulfate, filtered and concentrated in vacuo. The resulting solid was used without further purification (0.84 g, 100% yield): ¹H-NMR (400 MHz; CDCl₃) δ 8.10 (d, J=4.9 Hz, 1H), 7.23-7.18 (m, 1H), 7.16-7.07 (m, 2H), 6.93 (d, J=4.9 Hz, 1H), 3.70-3.66 (m, 2H), 2.83-2.76 (m, 1H), 2.36-2.26 (m, 2H), 2.18-2.08 (m, 2H), 2.04-1.98 (m, 2H), 1.96-1.82 (m, 2H); MS (ES+) m/z 325.2 (M+1).

### Step 6. Preparation of N-[2-(4,4-difluorocyclohexyl)-4-(2,5-difluorophenyl)-3-pyridyl]-2-isopropyl-pyrimidine-5-carboxamide

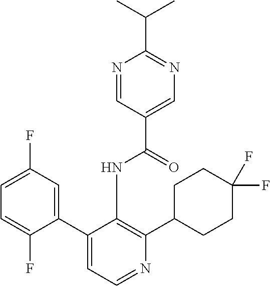 [SMILES: CC(C)c1ncc(C(=O)Nc2c(-c3cc(F)ccc3F)ccnc2C2CCC(F)(F)CC2)cn1]

To a mixture of 2-(4,4-difluorocyclohexyl)-4-(2,5-difluorophenyl)pyridin-3-amine (0.100 g, 0.31 mmol), 2-isopropylpyrimidine-5-carboxylic acid (0.077 g, 0.46 mmol) and 2-chloro-1-methylpyridinium iodide (0.20 g, 0.77 mmol) was added anhydrous tetrahydrofuran (6.2 mL). The solution was heated at 65° C. for 1 min before N-ethyl-N-isopropylpropan-2-amine (0.40 g, 3.1 mmol) was added. The reaction mixture was stirred at 65° C. for 3 h. The reaction mixture was cooled to ambient temperature and diluted with methanol (5 mL), and 10 M sodium hydroxide (2 mL). The mixture was stirred at ambient temperature for 30 min before it was diluted with ethyl acetate (150 mL). The organic layer was washed with saturated ammonium chloride solution (2×50 mL), dried over anhydrous magnesium sulfate, filtered and concentrated in vacuo. Purification of the residue by reverse phase column chromatography, eluting with 5 to 95% acetonitrile in water with 0.5% formic acid, followed by suspension in diethyl ether (10 mL) and filtration, provided the title compound as a colorless solid (0.019 g, 13% yield): ¹H-NMR (400 MHz; DMSO-d₆) δ 10.36 (s, 1H), 8.97 (s, 2H), 8.63 (d, J=4.9 Hz, 1H), 7.39-7.34 (m, 2H), 7.30-7.23 (m, 2H), 3.24-3.17 (m, 2H), 2.10-1.82 (m, 8H), 1.29 (d, J=6.9 Hz, 6H); MS (ES+) m/z 473.2 (M+1).

### Example 7

### Synthesis of N-[2-(4,4-difluorocyclohexyl)-4-(2,5-difluorophenyl)-3-pyridyl]-2-isopropoxy-pyrimidine-5-carboxamide

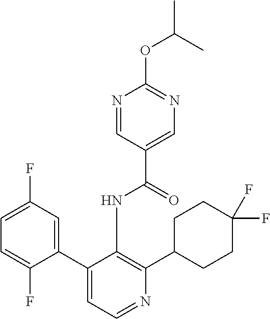 [SMILES: CC(C)Oc1ncc(C(=O)Nc2c(-c3cc(F)ccc3F)ccnc2C2CCC(F)(F)CC2)cn1]

To a mixture of 2-(4,4-difluorocyclohexyl)-4-(2,5-difluorophenyl)pyridin-3-amine (0.27 g, 0.31 mmol), 2-chloropyrimidine-5-carboxylic acid (0.20 g, 1.2 mmol) and 2-chloro-1-methylpyridinium iodide (0.53 g, 2.1 mmol) was added anhydrous tetrahydrofuran (17 mL). The solution was heated at 65° C. for 1 min before N-ethyl-N-isopropylpropan-2-amine (1.1 g, 8.3 mmol) was added. The reaction mixture was stirred at 65° C. for 3 h. The reaction mixture was cooled to ambient temperature before being diluted with ethyl acetate (100 mL). The organic layer was washed with saturated ammonium chloride solution (2×50 mL), dried over anhydrous magnesium sulfate, filtered and concentrated in vacuo. The residue was purified by column chromatography, eluting with 0 to 25% methanol in ethyl acetate. The solid was dissolved in a isopropanol (15 mL) and N,N-dimethylformamide. To the solution was added a 60% suspension of sodium hydride in mineral oil (0.32 g, 8.3 mmol). The reaction mixture was heated to 50° C. for 2 h. After cooling to ambient temperature, the reaction mixture was diluted with ethyl acetate (150 mL), washed with saturated ammonium chloride (2×50 mL), dried over anhydrous magnesium sulfate, filtered, and concentrated in vacuo. Purification of the residue by preparative HPLC, eluting with a gradient of 5 to 95% of acetonitrile in water containing 0.5% formic acid, provided the title compound as a colorless solid, which was triturated with diethyl ether (10 mL) and filtered (0.055 g, 13% yield): ¹H-NMR (400 MHz; DMSO-d₆) δ 10.20 (s, 1H), 8.87 (d, J=2.6 Hz, 2H), 8.62 (d, J=4.9 Hz, 1H), 7.38-7.37 (m, 1H), 7.33 (dd, J=9.2, 4.6 Hz, 1H), 7.30-7.21 (m, 2H), 5.27 (quintet, J=6.2 Hz, 1H), 3.21-3.17 (m, 1H), 2.12-2.06 (m, 2H), 1.99-1.83 (m, 6H), 1.34 (d, J=6.2 Hz, 6H); MS (ES+) m/z 489.2 (M+1).

### Example 8

### Synthesis of N-[2-(4,4-difluorocyclohexyl)-4-(2,5-difluorophenyl)-3-pyridyl]-6-methoxy-pyridine-3-carboxamide

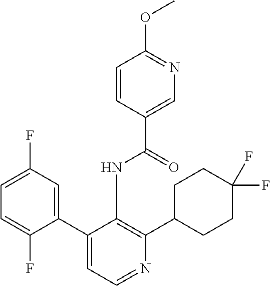 [SMILES: COc1ccc(C(=O)Nc2c(-c3cc(F)ccc3F)ccnc2C2CCC(F)(F)CC2)cn1]

### Step 1. Preparation of 6-chloro-N-[2-(4,4-difluorocyclohexyl)-4-(2,5-difluorophenyl)-3-pyridyl]pyridine-3-carboxamide

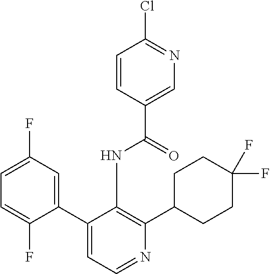 [SMILES: O=C(Nc1c(-c2cc(F)ccc2F)ccnc1C1CCC(F)(F)CC1)c1ccc(Cl)nc1]

To a mixture of 2-(4,4-difluorocyclohexyl)-4-(2,5-difluorophenyl)pyridin-3-amine (0.25 g, 0.77 mmol), 6-chloronicotinic acid (0.18 g, 1.2 mmol) and 2-chloro-1-methylpyridinium iodide (0.49 g, 1.9 mmol) was added anhydrous tetrahydrofuran (15 mL). The solution was heated at 55° C. for 1 min before N-ethyl-N-isopropylpropan-2-amine (1.0 g, 7.7 mmol) was added. The reaction mixture was stirred at 55° C. for 3 h. The reaction mixture was cooled to ambient temperature and diluted with methanol (5 mL), and 10 M sodium hydroxide (2 mL). The mixture was stirred for 18 h. The reaction was diluted with ethyl acetate (100 mL), washed with saturated ammonium chloride (2×20 mL), dried over anhydrous magnesium sulfate, filtered, and concentrated in vacuo. Purification of the residue by column chromatography, eluting with a gradient of 10 to 90% of ethyl acetate in heptane, provided the title compound as a colorless solid (0.18 g, 50% yield): ¹H-NMR (400 MHz; DMSO-d₆) δ 8.72-8.70 (m, 1H), 8.68-8.66 (m, 1H), 8.06-8.02 (m, 1H), 7.55-7.53 (m, 1H), 7.48-7.45 (m, 1H), 7.24-7.22 (m, 1H), 7.17-7.07 (m, 3H), 3.04-2.97 (m, 1H), 2.30-2.12 (m, 4H), 1.99-1.93 (m, 2H), 1.88-1.71 (m, 2H); MS (ES+) m/z 464.4 (M+1), 466.2 (M+1).

### Step 2. Preparation of N-[2-(4,4-difluorocyclohexyl)-4-(2,5-difluorophenyl)-3-pyridyl]-6-methoxy-pyridine-3-carboxamide

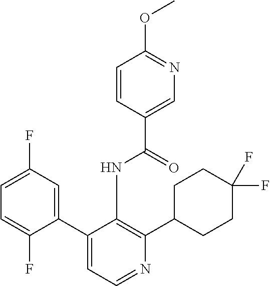 [SMILES: COc1ccc(C(=O)Nc2c(-c3cc(F)ccc3F)ccnc2C2CCC(F)(F)CC2)cn1]

To a mixture of 6-chloro-N-[2-(4,4-difluorocyclohexyl)-4-(2,5-difluorophenyl)-3-pyridyl]pyridine-3-carboxamide (0.090 g, 0.19 mmol) in anhydrous methanol (4.0 mL) was added a 60% dispersion of sodium hydride in mineral oil (0.021 g, 0.57 mmol). The solution was heated at 100° C. for 24 h. The reaction mixture was cooled to ambient temperature and diluted with ethyl acetate (100 mL). The organic layer was washed with saturated ammonium chloride (2×50 mL), dried over anhydrous magnesium sulfate, filtered and concentrated in vacuo. Purification of the residue by column chromatography, eluting with 10 to 90% ethyl acetate in heptane, followed by preparative HPLC, eluting with a gradient of 10 to 90% of acetonitrile in water containing 0.5% formic acid, provided the title compound as a colorless solid which was filtered from a 10:1 mixture of water and acetonitrile (0.009 g, 10% yield): ¹H-NMR (400 MHz; DMSO-d₆) δ 10.06 (s, 1H), 8.61 (d, J=4.9 Hz, 1H), 8.57 (d, J=2.2 Hz, 1H), 8.00 (dd, J=8.7, 2.5 Hz, 1H), 7.36 (d, J=4.8 Hz, 1H), 7.33 (td, J=9.1, 4.6 Hz, 1H), 7.24 (tdd, J=11.1, 7.6, 3.4 Hz, 2H), 6.90 (d, J=8.7 Hz, 1H), 3.91 (s, 3H), 3.20-3.15 (m, 1H), 2.11-2.08 (m, 2H), 1.97-1.86 (m, 6H); MS (ES+) m/z 460.2 (M+1).

### Example 9

### Synthesis of N-[4-(2,5-difluorophenyl)-2-(5,5-difluorotetrahydropyran-2-yl)-3-pyridyl]-2-isopropyl-pyrimidine-5-carboxamide

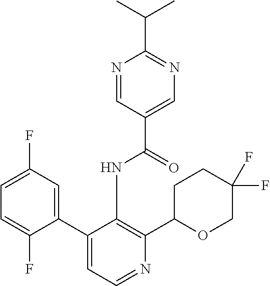 [SMILES: CC(C)c1ncc(C(=O)Nc2c(-c3cc(F)ccc3F)ccnc2C2CCC(F)(F)CO2)cn1]

### Step 1. Preparation of tert-butyl N-[4-(2,5-difluorophenyl)-2-(5,5-difluorotetrahydropyran-2-yl)-3-pyridyl]carbamate

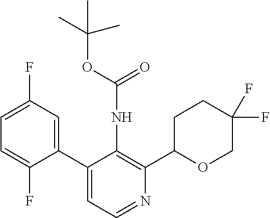 [SMILES: CC(C)(C)OC(=O)Nc1c(-c2cc(F)ccc2F)ccnc1C1CCC(F)(F)CO1]

To tert-butyl N-[2-chloro-4-(2,5-difluorophenyl)-3-pyridyl]carbamate (0.69 g, 2.0 mmol) was added 4,4-difluorocyclohexanecarboxylic acid (0.33 g, 2.0 mmol), (4,4″-Di-t-butyl-2,2″-bipyridine)bis[3,5-difluoro-2-[5-trifluoromethyl-2-pyridinyl-kN)phenyl-kC]iridium(III) hexafluorophosphate (0.017 g, 0.015 mmol), dichloro(dimethoxyethane)nickel (0.034 g, 0.15 mmol), 4-tert-butyl-2-(4-tert-butyl-2-pyridyl)pyridine (0.062 g, 0.23 mmol), cesium carbonate (0.71 g, 2.2 mmol), and N,N-dimethylformamide (26 mL). The headspace of the vial was replaced with nitrogen, the vial was sealed, and the reaction mixture was stirred in front of Kessil PR160L lights (440 nm) for 24 h. The reaction mixture was diluted with ethyl acetate (250 mL) and washed with saturated ammonium chloride solution (2×50 mL), dried over anhydrous magnesium sulfate, filtered and concentrated in vacuo. Purification of the residue by column chromatography, eluting with 0-50% ethyl acetate in heptane, afforded the title compound as a yellow oil (0.43 g, 65% yield): MS (ES+) m/z 427.2 (M+1).

### Step 2. Preparation of 4-(2,5-difluorophenyl)-2-(5,5-difluorotetrahydropyran-2-yl)pyridin-3-amine

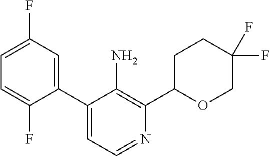 [SMILES: Nc1c(-c2cc(F)ccc2F)ccnc1C1CCC(F)(F)CO1]

To tert-butyl N-[4-(2,5-difluorophenyl)-2-(5,5-difluorotetrahydropyran-2-yl)-3-pyridyl]carbamate (0.43 g, 1.0 mmol) was added a 4 M solution of hydrochloric acid in 1,4-dioxane (10 mL). The reaction mixture was stirred for 3 h. The reaction mixture was diluted with ethyl acetate (100 mL), washed with saturated sodium bicarbonate (2×50 mL), dried over anhydrous magnesium sulfate, filtered and concentrated in vacuo. The resulting brown oil was used without further purification (0.36 g, 100% yield): ¹H-NMR (400 MHz; CDCl₃) δ 7.85 (d, J=4.8 Hz, 1H), 7.43-7.31 (m, 2H), 7.25 (ddd, J=8.8, 5.6, 3.1 Hz, 1H), 7.01 (d, J=4.8 Hz, 1H), 4.97 (s, 2H), 4.81 (dd, J=10.6, 1.9 Hz, 1H), 4.00-3.90 (m, 2H), 2.41-2.13 (m, 3H), 2.02-1.97 (m, 1H); MS (ES+) m/z 327.2 (M+1).

### Step 3. Preparation of N-[4-(2,5-difluorophenyl)-2-(5,5-difluorotetrahydropyran-2-yl)-3-pyridyl]-2-isopropyl-pyrimidine-5-carboxamide

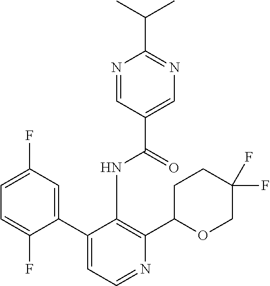 [SMILES: CC(C)c1ncc(C(=O)Nc2c(-c3cc(F)ccc3F)ccnc2C2CCC(F)(F)CO2)cn1]

To a mixture of 4-(2,5-difluorophenyl)-2-(5,5-difluorotetrahydropyran-2-yl)pyridin-3-amine (0.065 g, 0.20 mmol), 2-isopropylpyrimidine-5-carboxylic acid (0.043 g, 0.26 mmol) and 2-chloro-1-methylpyridinium iodide (0.13 g, 0.51 mmol) was added anhydrous tetrahydrofuran (3.3 mL). The solution was heated at 60° C. for 1 min before N-ethyl-N-isopropylpropan-2-amine (0.26 g, 2.0 mmol) was added. The reaction mixture was stirred at 60° C. for 3 h. The reaction mixture was cooled to ambient temperature and diluted with methanol (3 mL), and 10 M sodium hydroxide (2 mL). The mixture was stirred for 20 min at ambient temperature before it was diluted with ethyl acetate (100 mL). The organic layer was washed with saturated ammonium chloride solution (2×50 mL), dried over anhydrous magnesium sulfate, filtered and concentrated in vacuo. Purification of the residue by column chromatography, eluting with 10 to 100% of ethyl acetate in heptane, provided the title compound as a colorless solid (0.042 g, 44% yield): ¹H-NMR (400 MHz; DMSO-d₆) δ 10.39 (s, 1H), 8.98 (s, 2H), 8.68 (d, J=4.9 Hz, 1H), 7.54-7.53 (m, 1H), 7.40-7.34 (m, 1H), 7.32-7.26 (m, 2H), 4.95-4.92 (m, 1H), 3.94-3.89 (m, 1H), 3.86-3.74 (m, 1H), 3.20 (dt, J=13.8, 6.9 Hz, 1H), 2.33-2.21 (m, 3H), 1.98-1.95 (m, 1H), 1.29 (d, J=6.9 Hz, 6H); MS (ES+) m/z 475.2 (M+1).

### Example 10

### Synthesis of 2-isopropyl-N-(4-phenyl-2-(tetrahydro-2H-pyran-4-yl)pyridin-3-yl)pyrimidine-5-carboxamide

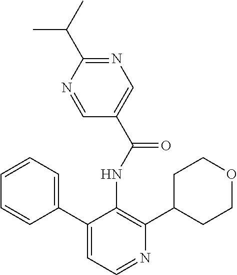 [SMILES: CC(C)c1ncc(C(=O)Nc2c(-c3ccccc3)ccnc2C2CCOCC2)cn1]

### Step 1. Preparation of 2-chloro-3-nitro-4-phenylpyridine

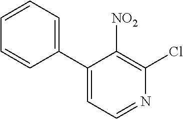 [SMILES: O=[N+]([O-])c1c(-c2ccccc2)ccnc1Cl]

A mixture of 2,4-dichloro-3-nitropyridine (3.00 g, 15.5 mmol) in dioxane (100 mL) and water (10 mL) was degassed with nitrogen for 10 minutes. To the reaction mixture was added phenylboronic acid (1.90 g, 15.5 mmol), dichloro 1,1′-bis(diphenylphosphino)ferrocene palladium (II) dichloromethane (1.32 g, 1.55 mmol), and potassium carbonate (3.22 g, 23.3 mmol). The reaction was stirred at 60° C. for 4 h. After cooling to ambient temperature, the mixture was filtered through a bed of diatomaceous earth (i.e., Celite®) and diluted with ethyl acetate (150 mL). The combined filtrate was washed with saturated ammonium chloride (3×100 mL), dried over anhydrous magnesium sulfate, filtered and concentrated in vacuo. The residue was purified by column chromatography, eluting with a gradient of 5 to 45% ethyl acetate in heptane, to afford the title compound as a colorless solid (2.95 g, 81% yield): MS (ES+) m/z 235.0 (M+1).

### Step 2. Preparation of 2-chloro-4-phenylpyridin-3-amine

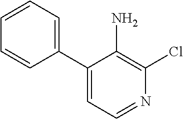 [SMILES: Nc1c(-c2ccccc2)ccnc1Cl]

To a mixture of 2-chloro-3-nitro-4-phenylpyridine (6.00 g, 25.6 mmol) in ethanol (51 mL) and water (51 mL) was added ammonium chloride (13.7 g, 256 mmol) and iron (7.14 g, 128 mmol). The reaction was stirred at 80° C. for 1.5 h. After cooling to ambient temperature, the mixture was diluted in ethyl acetate (600 mL) and filtered through a bed of diatomaceous earth (i.e., Celite®). The filtrate was washed with saturated sodium bicarbonate (2×200 mL), brine (200 mL), dried with magnesium sulfate, filtered and concentrated in vacuo, to afford the title compound as a colorless solid (5.30 g, 101% yield): MS (ES+) m/z 206.0 (M+1), 208.0 (M+1).

### Step 3. Preparation of N-(2-chloro-4-phenylpyridin-3-yl)-2-isopropylpyrimidine-5-carboxamide

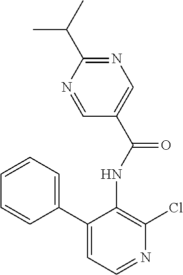 [SMILES: CC(C)c1ncc(C(=O)Nc2c(-c3ccccc3)ccnc2Cl)cn1]

To a mixture of 2-chloro-4-phenylpyridin-3-amine (2.50 g, 12.2 mmol) in anhydrous tetrahydrofuran (61 mL) and pyridine (9.80 mL, 122 mmol) was added 2-chloro-1-methylpyridinium iodide (9.36 g, 36.6 mmol) and 2-isopropylpyrimidine-5-carboxylic acid (2.23 g, 13.4 mmol). The reaction was stirred at 65° C. for 2 days. After cooling to ambient temperature, the mixture diluted in saturated ammonium chloride (100 mL), extracted with ethyl acetate (2×200 mL). The combined organic phase was washed with saturated ammonium chloride (100 mL), dried with magnesium sulfate, filtered and concentrated in vacuo. The residue was purified by column chromatography, eluting with a gradient of 0 to 75% ethyl acetate in heptane, to afford the title compound as a yellow solid (2.85 g, 66% yield): ¹H NMR (300 MHz, CDCl₃) δ 8.96 (s, 2H), 8.41 (d, J=5.0 Hz, 1H), 7.58 (s, 1H), 7.41 (s, 5H), 7.32 (d, J=5.0 Hz, 1H), 3.27 (sept, J=6.8 Hz, 1H), 1.35 (d, J=6.9 Hz, 6H); MS (ES+) m/z 353.0 (M+1), 355.0 (M+1).

### Step 4. N-(2-(3,6-dihydro-2H-pyran-4-yl)-4-phenylpyridin-3-yl)-2-isopropylpyrimidine-5-carboxamide

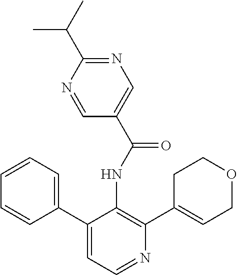 [SMILES: CC(C)c1ncc(C(=O)Nc2c(-c3ccccc3)ccnc2C2=CCOCC2)cn1]

A mixture of N-(2-chloro-4-phenylpyridin-3-yl)-2-isopropylpyrimidine-5-carboxamide (0.300 g, 0,850 mmol) in dioxane (8.5 mL) and water (2.1 mL) was degassed with nitrogen for 10 minutes. To the reaction mixture was added potassium phosphate tribasic (1.26 g, 5.95 mmol), 3,6-dihydro-2H-pyran-4-boronic acid pinacol ester (0.536 g, 2.55 mmol), palladium(II) acetate (0.057 g, 0.26 mmol), and tricyclohexylphosphine tetrafluoroborate (0.188 g, 0.510 mmol). The reaction was stirred at 110° C. for 20 h. After cooling to ambient temperature, the mixture was filtered through a bed of diatomaceous earth (i.e., Celite®) and the filter pad was washed with ethyl acetate (2×100 mL). The combined filtrate was washed with saturated ammonium chloride (2×75 mL) and concentrated in vacuo. The residue was purified by column chromatography, eluting with a gradient of 40 to 100% ethyl acetate in heptane, to afford the title compound as a colorless solid (0.325 g, 95% yield): ¹H NMR (300 MHz, CDCl₃) δ 10.30 (s, 1H), 8.94 (s, 2H), 8.60 (d, J=4.9 Hz, 1H), 7.48-7.36 (m, 6H), 6.08 (dt, J=2.8, 1.3 Hz, 1H), 4.08-4.07 (m, 2H), 3.76 (t, J=5.4 Hz, 2H), 3.18 (sept, J=6.9 Hz, 1H), 2.49-2.45 (m, 2H), 1.28 (d, J=6.9 Hz, 6H); MS (ES+) m/z 401.2 (M+1).

### Step 5. 2-isopropyl-N-(4-phenyl-2-(tetrahydro-2H-pyran-4-yl)pyridin-3-yl)pyrimidine-5-carboxamide

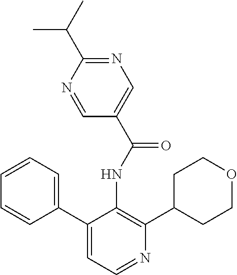 [SMILES: CC(C)c1ncc(C(=O)Nc2c(-c3ccccc3)ccnc2C2CCOCC2)cn1]

To a mixture of N-(2-(3,6-dihydro-2H-pyran-4-yl)-4-phenylpyridin-3-yl)-2-isopropylpyrimidine-5-carboxamide (0.078 g, 0.19 mmol) in methanol (1.0 mL) and ethyl acetate (1.0 mL) was added ammonium formate (0.123 g, 1.95 mmol) and 10% palladium on carbon (0.021 g). The reaction was stirred at 65° C. for 30 minutes. After cooling to ambient temperature, the mixture was diluted with ethyl acetate (50 mL), filtered through a bed of diatomaceous earth (i.e., Celite®) and the filtrate was concentrated in vacuo. The residue was purified by column chromatography, eluting with a gradient of 50 to 100% ethyl acetate in heptane, to afford the title compound as a colorless solid (0.037 g, 47% yield): ¹H NMR (300 MHz, DMSO-d₆) δ 10.31 (s, 1H), 8.99 (s, 2H), 8.61 (d, J=4.9 Hz, 1H), 7.48-7.35 (m, 5H), 7.32 (d, J=4.9 Hz, 1H), 3.92 (dd, J=11.1, 3.9 Hz, 2H), 3.39 (t, J=11.6 Hz, 2H), 3.27-3.15 (m, 2H), 1.90 (qd, J=12.3, 4.1 Hz, 2H), 1.62 (d, J=12.5 Hz, 2H), 1.28 (d, J=6.9 Hz, 6H); MS (ES+) m/z 403.2 (M+1).

### Example 11

### Synthesis of (±)-2-isopropyl-N-(4-phenyl-2-(tetrahydrofuran-2-yl)pyridin-3-yl)pyrimidine-5-carboxamide

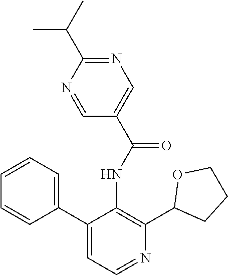 [SMILES: CC(C)c1ncc(C(=O)Nc2c(-c3ccccc3)ccnc2C2CCCO2)cn1]

### Step 1. Preparation of 2-(furan-2-yl)-3-nitro-4-phenylpyridine

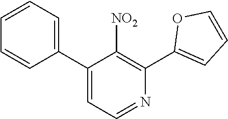 [SMILES: O=[N+]([O-])c1c(-c2ccccc2)ccnc1-c1ccco1]

A mixture of 2-chloro-3-nitro-4-phenylpyridine (0.500 g, 2.13 mmol) in dioxane (11 mL) and water (3.6 mL) was degassed with nitrogen for 10 minutes. To the reaction mixture was added potassium carbonate (0.442 g, 3.20 mmol), 2-furanboronic acid (0.715 g, 2.55 mmol), and 1,1′-bis(diphenylphosphino)ferrocene palladium (II) dichloromethane (0.183 g, 0.213 mmol). The reaction was stirred at 100° C. for 18 h. After cooling to ambient temperature, the mixture was filtered through a bed of diatomaceous earth (i.e., Celite®) and the filter pad was washed with ethyl acetate (2×100 mL). The combined filtrate was washed with saturated ammonium chloride (2×75 mL), dried with magnesium sulfate, filtered, and concentrated in vacuo. The residue was purified by column chromatography, eluting with a gradient of 0 to 30% ethyl acetate in heptane, to afford the title compound as a yellow solid (0.540 g, 95% yield): MS (ES+) m/z 267.0 (M+1).

### Step 2. Preparation of 2-(furan-2-yl)-4-phenylpyridin-3-amine

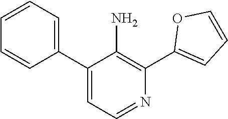 [SMILES: Nc1c(-c2ccccc2)ccnc1-c1ccco1]

To a mixture of 2-(furan-2-yl)-3-nitro-4-phenylpyridine (0.540 g, 2.03 mmol) in methanol (10.0 mL) and ethyl acetate (10.0 mL) was added ammonium formate (2.56 g, 40.6 mmol) and 10% palladium on carbon (0.108 g). The reaction was stirred at 65° C. for 1.5 h. After cooling to ambient temperature, the mixture was diluted in ethyl acetate (300 mL) and filtered through a bed of diatomaceous earth (i.e., Celite®). The filtrate was washed with saturated sodium bicarbonate (2×100 mL), dried with magnesium sulfate, filtered, and concentrated in vacuo. The residue was purified by column chromatography, eluting with a gradient of 5 to 100% ethyl acetate in heptane, to afford the title compound as a red oil (0.392 g, 82% yield): MS (ES+) m/z 237.2 (M+1).

### Step 3. Preparation of (±)-4-phenyl-2-(tetrahydrofuran-2-yl)pyridin-3-amine

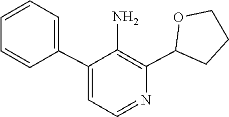 [SMILES: Nc1c(-c2ccccc2)ccnc1C1CCCO1]

To a mixture of 2-(furan-2-yl)-4-phenylpyridin-3-amine (0.392 g, 1.66 mmol) in methanol (15.0 mL) was added 10% palladium on carbon (0.160 g) and formic acid (0.60 mL, 20 mmol). The reaction mixture was stirred under 50 psi of hydrogen for 3.5 h. The mixture was diluted with ethyl acetate (150 mL) and filtered through a bed of diatomaceous earth (i.e., Celite®). The filtrate was washed with saturated sodium bicarbonate (2×50 mL), dried with magnesium sulfate, filtered, and concentrated in vacuo. The residue was purified by column chromatography, eluting with a gradient of 5 to 60% ethyl acetate in heptane, to afford the title compound as a yellow oil (0.218 g, 55% yield): MS (ES+) m/z 241.0 (M+1).

### Step 4. Preparation of (±)-2-isopropyl-N-(4-phenyl-2-(tetrahydrofuran-2-yl)pyridin-3-yl)pyrimidine-5-carboxamide

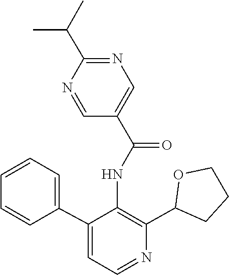 [SMILES: CC(C)c1ncc(C(=O)Nc2c(-c3ccccc3)ccnc2C2CCCO2)cn1]

To a mixture of 4-phenyl-2-(tetrahydrofuran-2-yl)pyridin-3-amine (0.063 g, 0.26 mmol) in tetrahydrofuran (2.6 mL) was added N,N-diisopropylethylamine (0.46 mL, 2.6 mmol), 2-chloro-1-methylpyridinium iodide (0.201 g, 0.787 mmol), 2-isopropylpyrimidine-5-carboxylic acid (0.052 g, 0.31 mmol). The reaction was stirred at 65° C. for 18 h. After cooling to ambient temperature, the mixture was diluted in ethyl acetate (100 mL) and washed with saturated ammonium chloride (3×25 mL), dried with magnesium sulfate, filtered and concentrated in vacuo. The residue was purified by column chromatography, eluting with a gradient of 5 to 100% ethyl acetate in heptane, to afford the title compound as a colorless solid (0.056 g, 55% yield): ¹H NMR (300 MHz, DMSO-d₆) δ 10.37 (s, 1H), 9.01 (s, 2H), 8.61 (d, J=4.9 Hz, 1H), 7.49-7.33 (m, 6H), 5.17 (t, J=6.8 Hz, 1H), 3.94 (q, J=7.3 Hz, 1H), 3.78 (td, J=7.6, 5.5 Hz, 1H), 3.19 (sept, J=6.9 Hz, 1H), 2.24-1.97 (m, 3H), 1.94-1.82 (m, 1H), 1.27 (d, J=6.9 Hz, 6H); MS (ES+) m/z 389.2 (M+1).

### Example 12

### Synthesis of (±)-N-(2-(3-oxabicyclo[4.1.0]heptan-6-yl)-4-phenylpyridin-3-yl)-2-isopropylpyrimidine-5-carboxamide

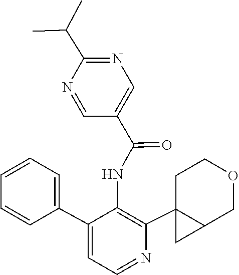 [SMILES: CC(C)c1ncc(C(=O)Nc2c(-c3ccccc3)ccnc2C23CCOCC2C3)cn1]

### Step 1. Preparation of (±)-2-(3-oxabicyclo[4.1.0]heptan-6-yl)-3-nitro-4-phenylpyridine

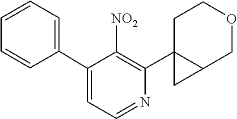 [SMILES: O=[N+]([O-])c1c(-c2ccccc2)ccnc1C12CCOCC1C2]

A mixture of 2-chloro-3-nitro-4-phenylpyridine (0.200 g, 0.852 mmol), cesium carbonate (0.555 g, 1.70 mmol), and (3-oxabicyclo[4.1.0]heptan-6-yl)trifluoroborate potassium salt (0.209 g, 1.02 mmol) in toluene (2.1 mL) and water (0.21 mL) was degassed with nitrogen for 10 minutes (note: the BF₃K salt was synthesized following a literature procedure described in J. Med. Chem. 2019, 62, 6972.). To the reaction mixture was added 1,1′-bis(diphenylphosphino)ferrocene palladium (II) dichloromethane (0.073 g, 0.085 mmol), and the reaction was stirred at 110° C. for 18 h. After cooling to ambient temperature, the mixture was filtered through a bed of diatomaceous earth (i.e., Celite®) and the filter pad was washed with ethyl acetate (2×75 mL). The combined filtrate was washed with saturated ammonium chloride (2×50 mL), dried with magnesium sulfate, filtered, and concentrated in vacuo. The residue was purified by column chromatography, eluting with a gradient of 0 to 30% ethyl acetate in heptane, to afford the title compound as a yellow oil (0.062 g, 25% yield): MS (ES+) m/z 297.0 (M+1).

### Step 2. Preparation of (±)-2-(3-oxabicyclo[4.1.0]heptan-6-yl)-4-phenylpyridin-3-amine

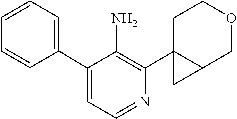 [SMILES: Nc1c(-c2ccccc2)ccnc1C12CCOCC1C2]

A mixture of 2-(3-oxabicyclo[4.1.0]heptan-6-yl)-3-nitro-4-phenylpyridine (0.062 g, 0.21 mmol) in methanol (2.1 mL) and ethyl acetate (2.1 mL) was flushed with hydrogen for 10 minutes. To the reaction mixture was added 10% palladium on carbon (0.050 g). The reaction was stirred at ambient temperature for 30 minutes. The mixture was diluted with ethyl acetate (50 mL), filtered through a bed of diatomaceous earth (i.e., Celite®), and concentrated in vacuo to afford a colorless oil which was used in the next step without further purification: MS (ES+) m/z 267.2 (M+1).

### Step 3. Preparation of (±)-N-(2-(3-oxabicyclo[4.1.0]heptan-6-yl)-4-phenylpyridin-3-yl)-2-isopropylpyrimidine-5-carboxamide

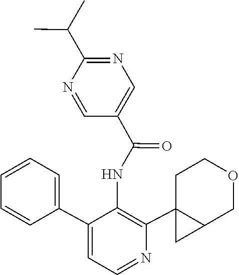 [SMILES: CC(C)c1ncc(C(=O)Nc2c(-c3ccccc3)ccnc2C23CCOCC2C3)cn1]

To a mixture of 2-(3-oxabicyclo[4.1.0]heptan-6-yl)-4-phenylpyridin-3-amine in anhydrous tetrahydrofuran (4.2 mL) was added N,N-diisopropylethylamine (0.37 mL, 2.1 mmol), 2-chloro-1-methylpyridinium iodide (0.161 g, 0.629 mmol), 2-isopropylpyrimidine-5-carboxylic acid (0.042 g, 0.25 mmol). The reaction was stirred at 65° C. for 1 h. After cooling to ambient temperature, the mixture was diluted in ethyl acetate (100 mL) and washed with saturated ammonium chloride (2×25 mL), dried with magnesium sulfate, filtered and concentrated in vacuo. The residue was purified by column chromatography, eluting with a gradient of 20 to 100% ethyl acetate in heptane, to afford a colorless solid. Further purification of the residue by reverse-phase column chromatography, using a gradient of 10 to 75% acetonitrile in water containing 0.5% formic acid as eluent, afforded the title compound as a colorless solid (0.019 g, 22% yield over 2 steps): ¹H NMR (300 MHz, DMSO-d₆) δ 10.28 (s, 1H), 9.03 (s, 2H), 8.51 (d, J=4.9 Hz, 1H), 7.50-7.35 (m, 6H), 3.85-3.58 (m, 2H), 3.51-3.44 (m, 1H), 3.25-3.01 (m, 2H), 2.02-1.89 (m, 2H), 1.29 (d, J=6.9 Hz, 6H), 0.87-0.72 (m, 2H); MS (ES+) m/z 415.2 (M+1).

### Example 13

### Synthesis of (±)-tert-butyl 2-(3-(2-isopropylpyrimidine-5-carboxamido)-4-phenylpyridin-2-yl)pyrrolidine-1-carboxylate

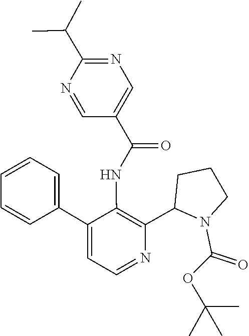 [SMILES: CC(C)c1ncc(C(=O)Nc2c(-c3ccccc3)ccnc2C2CCCN2C(=O)OC(C)(C)C)cn1]

To a mixture of N-(2-chloro-4-phenylpyridin-3-yl)-2-isopropylpyrimidine-5-carboxamide (0.142 g, 0.402 mmol), cesium carbonate (0.196 g, 1.70 mmol), nickel(II) chloride dimethoxyethane adduct (0.009 g, 0.04 mmol), 4-tert-butyl-2-(4-tert-butyl-2-pyridyl)pyridine (0.017 g, 0.060 mmol), and (4,4″-di-tert-butyl-2,2″-bipyridine)bis[3,5-difluoro-2-[5-trifluoromethyl-2-pyridinyl-kN)phenyl-kC]iridium(II) hexafluorophosphate (0.005 g, 0.004 mmol) was added N,N-dimethylformamide (10 mL) and the headspace was flushed with nitrogen for 10 seconds. The vial was sealed and placed in front of 4 Kessil PR160L lights (440 nm) for 18 h. The reaction mixture was diluted with ethyl acetate (100 mL) and the organic phase was washed with saturated sodium bicarbonate (30 mL), water (3×30 mL), and brine (30 mL). The organic phase was dried with magnesium sulfate, filtered, and concentrated in vacuo. Purification of the residue by reverse-phase column chromatography, using a gradient of 0 to 100% acetonitrile in water containing 0.5% formic acid as eluent, afforded the title compound as a colorless solid (0.176 g, 90% yield): ¹H NMR (400 MHz, DMSO-d₆, rotamers are present) δ 10.52 (s, 0.4H), 10.20 (s, 0.5H), 9.02 (d, J=1.3 Hz, 1H), 8.94 (d, J=1.1 Hz, 1H), 8.55-8.53 (m, 1H), 7.49-7.33 (m, 6H), 5.18 (d, J=7.5 Hz, 0.5H), 5.08 (d, J=5.4 Hz, 0.5H), 3.59-3.52 (m, 1H), 3.45-3.37 (m, 1H), 3.19 (sept, J=6.9 Hz, 1H), 2.33-2.14 (m, 1H), 1.98-1.75 (m, 3H), 1.37 (s, 4.5H), 1.28 (d, J=6.9 Hz, 6H), 1.10 (s, 4.5H); MS (ES+) m/z 488.2 (M+1).

### Example 14

### Synthesis of N-(4-(2-fluorophenyl)-2-(tetrahydro-2H-pyran-4-yl)pyridin-3-yl)-2-isopropylpyrimidine-5-carboxamide

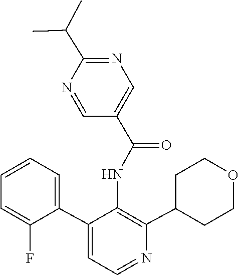 [SMILES: CC(C)c1ncc(C(=O)Nc2c(-c3ccccc3F)ccnc2C2CCOCC2)cn1]

### Step 1. Preparation of 2-chloro-4-(2-fluorophenyl)pyridin-3-amine

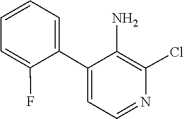 [SMILES: Nc1c(-c2ccccc2F)ccnc1Cl]

To a mixture of 2-chloro-4-(2-fluorophenyl)-3-nitro-pyridine (3.29 g, 13.0 mmol) in ethanol (26 mL) and water (26 mL) was added ammonium chloride (6.97 g, 130 mmol) and iron (5.46 g, 97.7 mmol). The reaction was stirred at 80° C. for 3 h. After cooling to ambient temperature, the mixture was diluted in ethyl acetate (500 mL) and filtered through a bed of diatomaceous earth (i.e., Celite®). The filtrate was washed with saturated sodium bicarbonate (150 mL), water (150 mL), brine (150 mL), dried with magnesium sulfate, filtered and concentrated in vacuo, to afford the title compound as a colorless solid (1.97 g, 68% yield): MS (ES+) m/z 223.0 (M+1), 225.0 (M+1).

### Step 2. Preparation of N-(2-chloro-4-(2-fluorophenyl)pyridin-3-yl)-2-isopropylpyrimidine-5-carboxamide

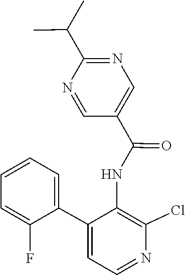 [SMILES: CC(C)c1ncc(C(=O)Nc2c(-c3ccccc3F)ccnc2Cl)cn1]

To a mixture of 2-chloro-4-(2-fluorophenyl)pyridin-3-amine (1.00 g, 4.49 mmol) in anhydrous tetrahydrofuran (30 mL) and pyridine (3.6 mL, 45 mmol) was added 2-chloro-1-methylpyridinium iodide (3.44 g, 13.5 mmol) and 2-isopropylpyrimidine-5-carboxylic acid (0.821 g, 4.94 mmol). The reaction was stirred at 65° C. for 2 days. After cooling to ambient temperature, the mixture diluted in saturated ammonium chloride (100 mL), extracted with ethyl acetate (2×200 mL). The combined organic phase was washed with saturated ammonium chloride (100 mL), dried with magnesium sulfate, filtered and concentrated in vacuo. The residue was purified by column chromatography, eluting with a gradient of 10 to 75% ethyl acetate in heptane, to afford the title compound as a yellow solid (1.700 g, 102% yield): ¹H NMR (400 MHz, CDCl₃) δ 8.94 (s, 2H), 8.45 (d, J=5.0 Hz, 1H), 7.75 (s, 1H), 7.42-7.33 (m, 3H), 7.25-7.21 (m, 1H), 7.15 (dd, J=10.3, 8.5 Hz, 1H), 3.27 (sept, J=6.9 Hz, 1H), 1.35 (d, J=6.9 Hz, 6H); ¹⁹F NMR (400 MHz, CDCl₃) δ −114.9; MS (ES+) m/z 371.0 (M+1), 373.2 (M+1).

### Step 3. Preparation of N-(4-(2-fluorophenyl)-2-(tetrahydro-2H-pyran-4-yl)pyridin-3-yl)-2-isopropylpyrimidine-5-carboxamide

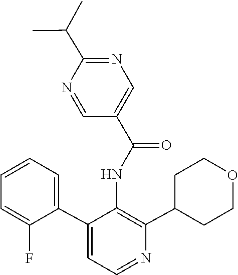 [SMILES: CC(C)c1ncc(C(=O)Nc2c(-c3ccccc3F)ccnc2C2CCOCC2)cn1]

A mixture of N-(2-chloro-4-(2-fluorophenyl)pyridin-3-yl)-2-isopropylpyrimidine-5-carboxamide (0.100 g, 0.270 mmol), cesium carbonate (0.132 g, 0.405 mmol), nickel(II) chloride dimethoxyethane adduct (0.006 g, 0.03 mmol), 4-tert-butyl-2-(4-tert-butyl-2-pyridyl)pyridine (0.011 g, 0.041 mmol), (4,4″-di-tert-butyl-2,2″-bipyridine)bis[3,5-difluoro-2-[5-trifluoromethyl-2-pyridinyl-kN)phenyl-kC]iridium(II) hexafluorophosphate (0.003 g, 0.003 mmol) was dissolved in N,N-dimethylformamide (6.7 mL) and flushed with nitrogen for 10 seconds. The vial was sealed and placed in front of 4 Kessil PR160L lights (440 nm) for 4 h. The reaction mixture was diluted with ethyl acetate (100 mL) and the organic phase was washed with saturated sodium bicarbonate (30 mL), water (3×30 mL), and brine (30 mL). The organic extracts were dried with magnesium sulfate, filtered, and concentrated in vacuo. The residue was purified by reverse-phase column chromatography, using a gradient of 0 to 100% acetonitrile in water containing 0.5% formic acid as eluent, to afford a colorless solid. The residue was further purified by column chromatography, using a gradient of 50 to 100% ethyl acetate in heptane as eluent, to afford the title compound as a colorless solid (0.024 g, 21% yield): ¹H NMR (400 MHz, DMSO-d₆, rotamers are present) δ 10.32 (s, 1H), 8.92 (s, 2H), 8.63 (d, J=4.9 Hz, 1H), 7.44-7.34 (m, 3H), 7.31-7.26 (m, 1H), 7.23 (td, J=7.5, 0.9 Hz, 1H), 3.94-3.91 (m, 2H), 3.39 (t, J=11.6 Hz, 2H), 3.30-3.15 (m, 2H), 1.97-1.86 (m, 2H), 1.63 (d, J=12.1 Hz, 2H), 1.28 (d, J=6.9 Hz, 6H); MS (ES+) m/z 421.2 (M+1).

### Example 15-39

In a similar manner as described in EXAMPLE Error! Reference source not found. step 3, utilizing the appropriately substituted starting materials and intermediates, the following compounds were prepared:

| | | | MS | |
|---|---|---|---|---|
| Example | Structure | Amount | (ES+) | |
| No. | Name | (% Yield) | m/z | ¹H NMR; ¹⁹F NMR |
| 15 | 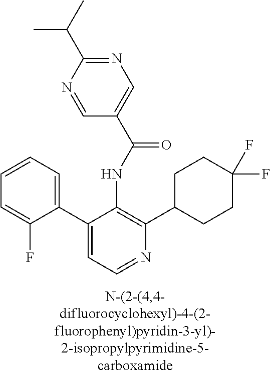 [SMILES: CC(C)c1ncc(C(=O)Nc2c(-c3ccccc3F)ccnc2C2CCC(F)(F)CC2)cn1] | 0.094 g (61%) | 455.2 (M + 1) | (400 MHz, DMSO-d₆) δ 10.30 (s, 1H), 8.93- 8.92 (m, 2H), 8.61 (d, J = 4.9 Hz, 1H), 7.44- 7.34 (m, 3H), 7.29 (ddd, J = 10.1, 8.6, 1.2 Hz, 1H), 7.23 (td, J = 7.5, 1.1 Hz, 1H), 3.24- 3.12 (m, 2H), 2.12- 1.81 (m, 8H), 1.28 (d, J = 6.9 Hz, 6H); (376 MHz, DMSO-d₆) δ −89.1 (d, J = 232 Hz), −99.8 (d, J = 232 Hz), −114.9 |
| 16 | 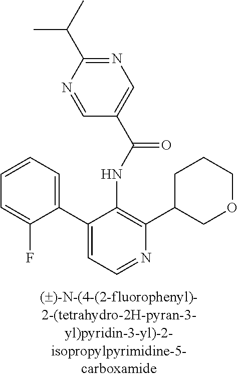 [SMILES: CC(C)c1ncc(C(=O)Nc2c(-c3ccccc3F)ccnc2C2CCCOC2)cn1] | 0.058 g (41%) | 421.2 (M + 1) | (400 MHz, DMSO-d₆) δ 10.37 (s, 1H), 8.94 (s, 2H), 8.61 (d, J = 4.9 Hz, 1H), 7.43-7.33 (m, 3H), 7.31-7.21 (m, 2H), 3.91-3.83 (m, 2H), 3.53 (t, J = 10.6 Hz, 1H), 3.38-3.29 (m, 1H), 3.28-3.15 (m, 2H), 1.99-1.86 (m, 2H), 1.67-1.55 (m, 2H), 1.28 (d, J = 6.9 Hz, 6H) |
| 17 | 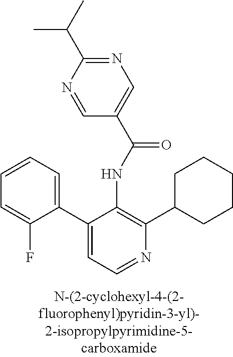 [SMILES: CC(C)c1ncc(C(=O)Nc2c(-c3ccccc3F)ccnc2C2CCCCC2)cn1] | 0.050 g (44%) | 419.2 (M + 1) | (400 MHz, DMSO-d₆) δ 10.30 (s, 1H), 8.92 (s, 2H), 8.59 (d, J = 4.9 Hz, 1H), 7.43-7.20 (m, 5H), 3.18 (sept, J = 6.9 Hz, 1H), 2.99 (tt, J = 11.4, 3.0 Hz, 1H), 1.72-1.70 (m, 4H), 1.70-1.56 (m, 3H), 1.35-1.17 (m, 9H) |
| 18 | 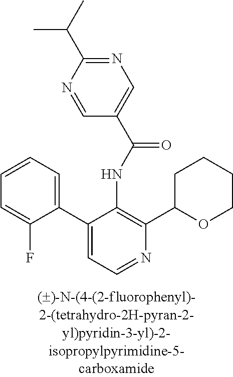 [SMILES: CC(C)c1ncc(C(=O)Nc2c(-c3ccccc3F)ccnc2C2CCCCO2)cn1] | 0.037 g (32%) | 421.2 (M + 1) | (400 MHz, DMSO-d₆) δ 10.32 (s, 1H), 8.95 (s, 2H), 8.62 (d, J = 4.9 Hz, 1H), 7.45-7.36 (m, 3H), 7.30-7.20 (m, 2H), 4.66 (dd, J = 10.8, 1.9 Hz, 1H), 3.94 (d, J = 11.0 Hz, 1H), 3.50-3.43 (m, 1H), 3.18 (sept, J = 6.9 Hz, 1H), 2.01-1.85 (m, 2H), 1.73-1.69 (m, 1H), 1.62-1.47 (m, 3H), 1.27 (d, J = 6.9 Hz, 6H) |
| 19 | 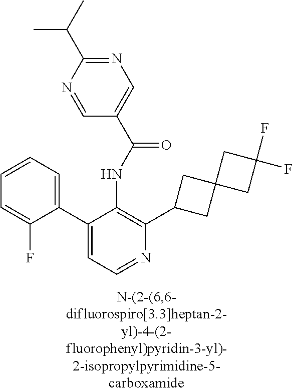 [SMILES: CC(C)c1ncc(C(=O)Nc2c(-c3ccccc3F)ccnc2C2CC3(C2)CC(F)(F)C3)cn1] | 0.032 g (51%) | 467.2 (M + 1) | (400 MHz, DMSO-d₆) δ 10.22 (s, 1H), 8.94 (s, 2H), 8.62 (d, J = 4.9 Hz, 1H), 7.43-7.21 (m, 5H), 3.83 (quintet, J = 8.6 Hz, 1H), 3.19 (sept, J = 6.9 Hz, 1H), 2.73-2.66 (m, 2H), 2.56-2.50 (m, 4H), 2.36 (td, J = 9.1, 2.2 Hz, 2H), 1.28 (d, J = 6.9 Hz, 6H) |
| 20 | 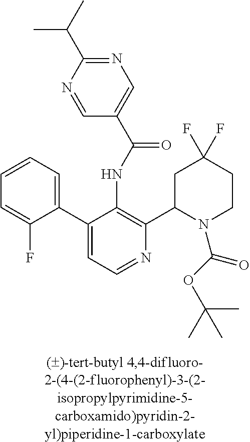 [SMILES: CC(C)c1ncc(C(=O)Nc2c(-c3ccccc3F)ccnc2C2CC(F)(F)CCN2C(=O)OC(C)(C)C)cn1] | 0.210 g (94%) | 556.2 (M + 1) | (400 MHz, DMSO-d₆) δ 10.33 (s, 1H), 8.90 (s, 2H), 8.56 (d, J = 4.9 Hz, 1H), 7.45-7.36 (m, 3H), 7.30 (t, J = 9.3 Hz, 1H), 7.24 (t, J = 7.5 Hz, 1H), 5.71 (s, 1H), 4.07 (d, J = 13.6 Hz, 1H), 3.96-3.86 (m, 1H), 3.18 (sept, J = 6.9 Hz, 1H), 2.50-2.40 (m, 2H), 2.14-1.93 (m, 2H), 1.27 (d, J = 6.9 Hz, 15H) |
| 21 | 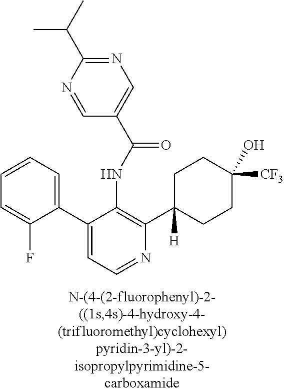 [SMILES: CC(C)c1ncc(C(=O)Nc2c(-c3ccccc3F)ccnc2[C@H]2CC[C@](O)(C(F)(F)F)CC2)cn1] | 0.016 g (15%) | 503.2 (M + 1) | (400 MHz, DMSO-d₆) δ 10.33 (s, 1H), 8.92 (s, 2H), 8.59 (d, J = 4.9 Hz, 1H), 7.43-7.20 (m, 5H), 5.72 (s, 1H), 3.29-3.13 (m, 2H), 2.19-2.16 (m, 2H), 1.86 (m, 4H), 1.56 (t, J = 6.2 Hz, 2H), 1.27 (d, J = 6.9 Hz, 6H) |
| 22 | 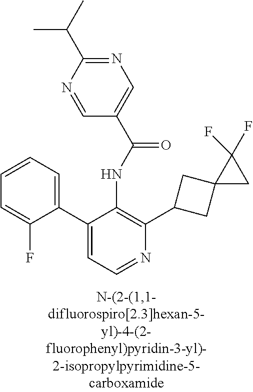 [SMILES: CC(C)c1ncc(C(=O)Nc2c(-c3ccccc3F)ccnc2C2CC3(C2)CC3(F)F)cn1] | 0.026 g (43%) | 453.2 (M + 1) | (400 MHz, DMSO-d₆, dr = 1:1.3 ) δ 10.27 (d, J = 2.1 Hz, 1H), 8.95 (d, J = 1.2 Hz, 2H), 8.67 (dd, J = 4.9, 1.7 Hz, 1H), 7.46-7.34 (m, 3H), 7.31-7.21 (m, 2H), 4.13-3.98 (m, 1H), 3.24-3.13 (m, 1H), 2.72-2.58 (m, 2H), 2.47-2.42 (m, 1H), 2.40-2.34 (m, 1H), 1.45 (t, J = 8.6 Hz, 1H), 1.37 (t, J = 8.6 Hz, 1H), 1.28 (d, J = 6.9 Hz, 6H); (376 MHz, DMSO-d₆, dr = 1:1.3) δ −114.9 (s, 1F), −115.0 (1.3F), −137.1 (2.1F), −137.3 (2.7F) |
| 23 | 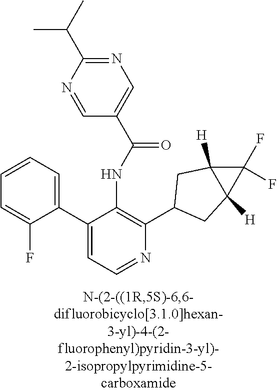 [SMILES: CC(C)c1ncc(C(=O)Nc2c(-c3ccccc3F)ccnc2C2C[C@@H]3[C@H](C2)C3(F)F)cn1] | 0.107 g (59%) | 453.2 (M + 1) | (400 MHz, DMSO-d₆) δ 10.34 (s, 1H), 8.90 (s, 2H), 8.64 (d, J = 4.9 Hz, 1H), 7.45-7.39 (m, 1H), 7.37-7.22 (m, 4H), 3.58-3.48 (m, 1H), 3.18 (sept, J = 6.9 Hz, 1H), 2.37 (s, 2H), 2.22 (d, J = 12.2 Hz, 4H), 1.27 (d, J = 6.9 Hz, 6H); (376 MHz, DMSO-d₆) δ −114.9, −122.8 (J = 156 Hz), −145.2 (J = 156 Hz) |
| 24 | 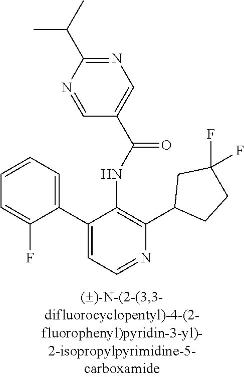 [SMILES: CC(C)c1ncc(C(=O)Nc2c(-c3ccccc3F)ccnc2C2CCC(F)(F)C2)cn1] | 0.046 g (52%) | 441.2 (M + 1) | (400 MHz, DMSO-d₆) δ 10.33 (s, 1H), 8.94 (s, 2H), 8.64 (d, J = 4.9 Hz, 1H), 7.44-7.34 (m, 3H), 7.32-7.21 (m, 2H), 3.83-3.77 (m, 1H), 3.22-3.13 (m, 1H), 2.64-2.53 (m, 1H), 2.47-2.35 (m, 1H), 2.33-1.93 (m, 4H), 1.27 (d, J = 6.9 Hz, 6H) |
| 25 | 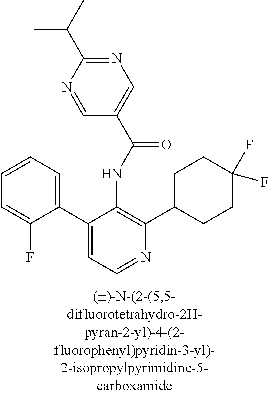 [SMILES: CC(C)c1ncc(C(=O)Nc2c(-c3ccccc3F)ccnc2C2CCC(F)(F)CC2)cn1] | 0.087 g (71%) | 457.2 (M + 1) | (400 MHz, DMSO-d₆) δ 10.35 (s, 1H), 8.94 (s, 2H), 8.66 (d, J = 4.9 Hz, 1H), 7.49 (dd, J = 4.9, 0.7 Hz, 1H), 7.45-7.36 (m, 2H), 7.32-7.27 (m, 1H), 7.23 (td, J = 7.5, 0.9 Hz, 1H), 4.91 (d, J = 10.3 Hz, 1H), 3.94- 3.88 (m, 1H), 3.79 (dt, J = 18.8, 12.6 Hz, 1H), 3.18 (sept, J = 6.9 Hz, 1H), 2.33-2.12 (m, 3H), 1.96 (d, J = 10.4 Hz, 1H), 1.28 (d, J = 6.9 Hz, 6H); (376 MHz, DMSO-d₆) δ −104.7, −115.1 |
| 26 | 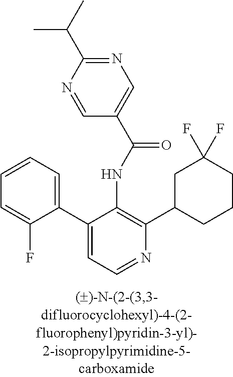 [SMILES: CC(C)c1ncc(C(=O)Nc2c(-c3ccccc3F)ccnc2C2CCCC(F)(F)C2)cn1] | 0.055 g (44%) | 455.2 (M + 1) | (400 MHz, DMSO-d₆) δ 10.40 (s, 1H), 8.92 (s, 2H), 8.63 (d, J = 4.9 Hz, 1H), 7.44-7.35 (m, 3H), 7.32-7.22 (m, 2H), 3.29 (s, 1H), 3.18 (sept, J = 6.9 Hz, 1H), 2.31-2.00 (m, 3H), 1.91-1.73 (m, 3H), 1.69-1.42 (m, 2H), 1.27 (d, J = 6.9 Hz, 6H) |
| 27 | 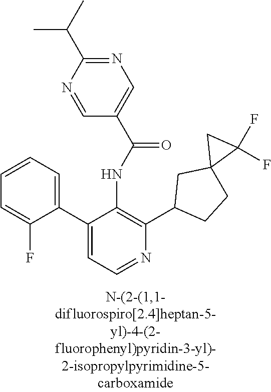 [SMILES: CC(C)c1ncc(C(=O)Nc2c(-c3ccccc3F)ccnc2C2CCC3(C2)CC3(F)F)cn1] | 0.047 g (53%) | 467.2 (M + 1) | (400 MHz, DMSO-d₆, dr = 1.5:1.0) δ 10.33 (s, 2.5H), 8.97-8.94 (m, 2H), 8.92-8.91 (m, 2.8H), 8.63 (d, J = 4.8 Hz, 1.4H), 8.63 (d, J = 4.8 Hz, 1H), 7.42-7.35 (m, 7.5H), 7.32-7.27 (m, 2.6H), 7.24 (t, J = 7.5, 1.5H), 7.24 (t, J = 7.5, 1H), 3.80 (quintet, J = 7.9 Hz, 1H), 3.63 (quintet, J = 8.3 Hz, 1.5H), 3.19 (sept, J = 6.9 Hz, 2.5H), 2.35- 1.86 (m, 12.5H), 1.82- 1.73 (m, 2.7H), 1.48- 1.35 (m, 5.4H), 1.28 (d, J = 6.9 Hz, 16.2H); (376 MHz, DMSO-d₆, dr = 1.5: 1.0) δ −114.9, −115.0, −134.3 (d, J = 147 Hz), 135.2 (d, J = 146 Hz), 135.5 (d, J = 146 Hz), 135.6 (d, J = 147 Hz) |
| 28 | 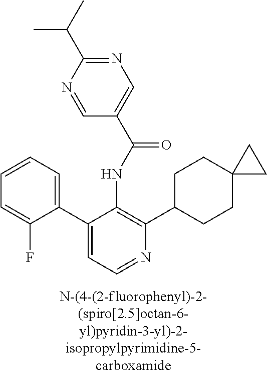 [SMILES: CC(C)c1ncc(C(=O)Nc2c(-c3ccccc3F)ccnc2C2CCC3(CC2)CC3)cn1] | 0.026 g (44%) | 445.2 (M + 1) | (400 MHz, DMSO-d₆) δ 10.32 (s, 1H), 8.92 (s, 2H), 8.60 (d, J = 4.9 Hz, 1H), 7.43-7.34 (m, 2H), 7.31-7.20 (m, 3H), 3.18 (sept, J = 6.9 Hz, 1H), 3.05-2.99 (m, 1H), 1.86-1.69 (m, 6H), 1.28 (d, J = 6.9 Hz, 6H), 0.97-0.84 (m, 2H), 0.30-0.21 (m, 4H); (376 MHz, DMSO-d₆) δ −114.9 |
| 29 | 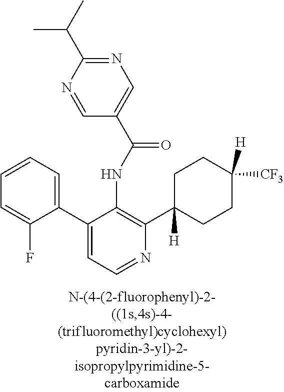 [SMILES: CC(C)c1ncc(C(=O)Nc2c(-c3ccccc3F)ccnc2[C@H]2CC[C@@H](C(F)(F)F)CC2)cn1] | 0.020 g (21%) | 487.2 (M + 1) | (400 MHz, DMSO-d₆) δ 10.32 (s, 1H), 8.92 (s, 2H), 8.60 (d, J = 4.9 Hz, 1H), 7.43-7.32 (m, 3H), 7.28 (t, J = 9.1 Hz, 1H), 7.24-7.20 (m, 1H), 3.17 (sept, J = 6.9 Hz, 1H), 3.04 (tt, J = 11.7, 3.3 Hz, 1H), 2.41-2.28 (m, 1H), 1.91 (m, 4H), 1.79- 1.66 (m, 2H), 1.41- 1.31 (m, 2H), 1.27 (d, J = 6.9 Hz, 6H); (376 MHz, DMSO-d₆) δ −72.2, −115.0 |
| 30 | 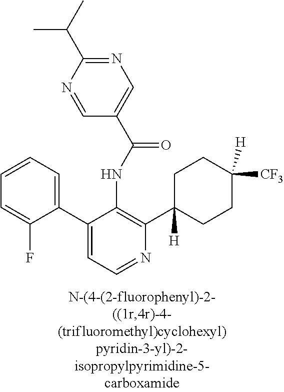 [SMILES: CC(C)c1ncc(C(=O)Nc2c(-c3ccccc3F)ccnc2[C@H]2CC[C@H](C(F)(F)F)CC2)cn1] | 0.018 g (18%) | 487.4 (M + 1) | (400 MHz, DMSO-d₆) δ 10.31 (s, 1H), 8.92 (s, 2H), 8.61 (d, J = 4.9 Hz, 1H), 7.43-7.32 (m, 3H), 7.30-7.26 (m, 1H), 7.22 (t, J = 7.5 Hz, 1H), 3.24-3.13 (m, 2H), 2.47-2.28 (m, 1H), 2.02-1.84 (m, 4H), 1.71-1.69 (m, 4H), 1.27 (d, J = 6.9 Hz, 6H); (376 MHz, DMSO-d₆) δ −67.9, −114.9 |
| 31 | 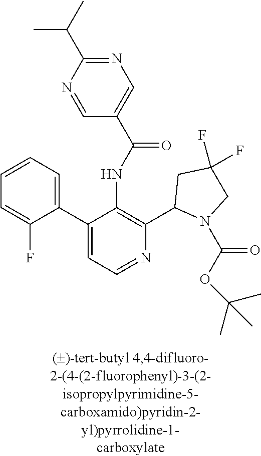 [SMILES: CC(C)c1ncc(C(=O)Nc2c(-c3ccccc3F)ccnc2C2CC(F)(F)CN2C(=O)OC(C)(C)C)cn1] | 0.211 g (96%) | 542.2 (M + 1) | (400 MHz, DMSO-d₆, rotamers are present) δ 10.52 (br s, 0.4H), 10.23 (s, 0.5H), 8.96 (s, 1H), 8.89 (s, 1H), 8.61 (t, J = 5.5 Hz, 1H), 7.48-7.23 (m, 5H), 5.46-5.29 (m, 1H), 4.00-3.87 (m, 2H), 3.20 (sept, J = 6.9 Hz, 1H), 3.12-2.90 (m, 1H), 2.47-2.37 (m, 1H), 1.38 (s, 4H), 1.28 (d, J = 6.9 Hz, 6H), 1.11 (s, 5H); (376 MHz, DMSO-d₆, rotamers are present) δ −94.1, −94.7, −95.1, −95.7, −97.3, −99.6, −114.8, −115.0 |
| 32 | 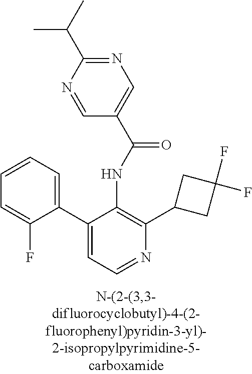 [SMILES: CC(C)c1ncc(C(=O)Nc2c(-c3ccccc3F)ccnc2C2CC(F)(F)C2)cn1] | 0.017 g (12%) | 427.4 (M + 1) | (400 MHz, DMSO-d₆) δ 10.32 (s, 1H), 8.95 (s, 2H), 8.67 (d, J = 4.9 Hz, 1H), 7.46-7.40 (m, 2H), 7.36 (td, J = 7.6, 1.7 Hz, 1H), 7.32- 7.22 (m, 2H), 3.75 (quintet, J = 8.6, 3.1 Hz, 1H), 3.19 (sept, J = 6.9 Hz, 1H), 3.02- 2.83 (m, 4H), 1.28 (d, J = 6.9 Hz, 6H); (376 MHz, DMSO-d₆) δ −79.7 (d, J = 190 Hz), −96.2 (d, J = 190 Hz), −114.9 |
| 33 | 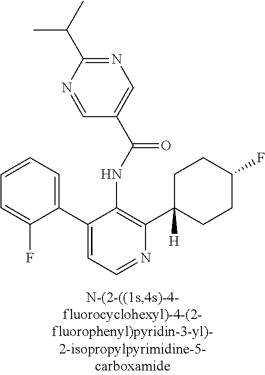 [SMILES: CC(C)c1ncc(C(=O)Nc2c(-c3ccccc3F)ccnc2[C@H]2CC[C@@H](F)CC2)cn1] | 0.026 g (13%) | 437.2 (M + 1) | (400 MHz, DMSO-d₆) δ 10.32 (s, 1H), 8.92 (s, 2H), 8.61 (d, J = 4.9 Hz, 1H), 7.42-7.20 (m, 5H), 4.87 (d, J = 48.5 Hz, 1H), 3.19 (sept, J = 6.9 Hz, 1H), 3.13-3.06 (m, 1H), 2.02-1.90 (m, 4H), 1.71-1.52 (m, 4H), 1.27 (d, J = 6.9 Hz, 6H); (376 MHz, DMSO-d₆) δ −114.9, −183.3 |
| 34 | 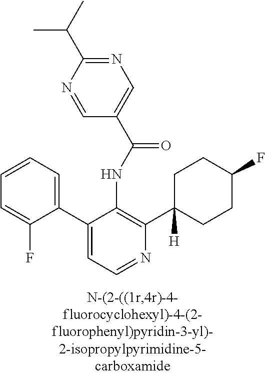 [SMILES: CC(C)c1ncc(C(=O)Nc2c(-c3ccccc3F)ccnc2[C@H]2CC[C@H](F)CC2)cn1] | 0.024 g (12%) | 437.4 (M + 1) | (400 MHz, DMSO-d₆) δ 10.32 (s, 1H), 8.93 (s, 2H), 8.59 (d, J = 4.9 Hz, 1H), 7.44-7.21 (m, 5H), 4.63 (dtt, J = 48.9, 10.4, 5.0 Hz, 1H), 3.19 (sept, J = 6.9 Hz, 1H), 3.03 (tt, J = 11.3, 3.8 Hz, 1H), 2.13-2.09 (m, 2H), 1.86-1.73 (m, 4H), 1.57-1.48 (m, 2H), 1.29 (d, J = 6.9 Hz, 6H); (376 MHz, DMSO-d₆) δ −114.9, −167.1 |
| 35 | 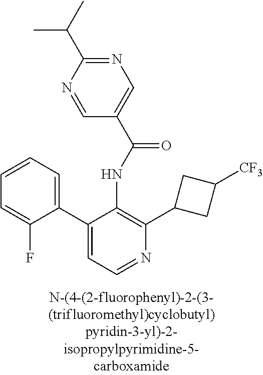 [SMILES: CC(C)c1ncc(C(=O)Nc2c(-c3ccccc3F)ccnc2C2CC(C(F)(F)F)C2)cn1] | 0.087 g (64%) | 459.2 (M + 1) | (400 MHz, DMSO-d₆, mixture of diastereomers, dr = 1.2:1) δ 10.27 (s, 0.85H), 8.96 (s, 2.2H), 8.93 (s, 1.8H), 8.68 (d, J = 5.0 Hz, 0.9H), 8.66 (d, J = 5.0 Hz, 1H), 7.45-7.33 (m, 6.0H), 7.31-7.22 (m, 4.1H), 4.00 (quintet, J = 8.3 Hz, 1H), 3.87 (quintet, J = 8.9 Hz, 1.2H), 3.24-3.12 (m, 4.2H), 2.63 (s, 2H), 2.35-2.34 (m, 6.1H), 1.28 (d, J = 6.9 Hz, 12.6H); (376 MHz, DMSO-d₆, mixture of diastereomers, dr = 1.2:1) δ −71.7, −72.3, −114.9 |
| 36 | 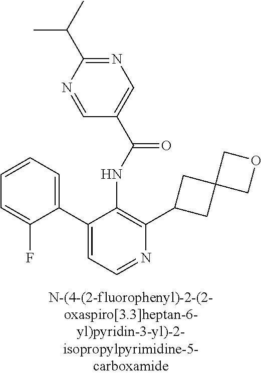 [SMILES: CC(C)c1ncc(C(=O)Nc2c(-c3ccccc3F)ccnc2C2CC3(COC3)C2)cn1] | 0.047 g (37%) | 433.2 (M + 1) | (400 MHz, DMSO-d₆) δ 10.21 (s, 1H), 8.93 (s, 2H), 8.61 (d, J = 4.9 Hz, 1H), 7.43-7.20 (m, 5H), 4.62 (s, 2H), 4.49 (s, 2H), 3.69 (quintet, J = 8.4 Hz, 1H), 3.18 (sept, J = 6.9 Hz, 1H), 2.52 (m 2H), 2.50 (m, 2H), 1.28 (d, J = 6.9 Hz, 6H); (376 MHz, DMSO-d₆) δ −115.0 |
| 37 | 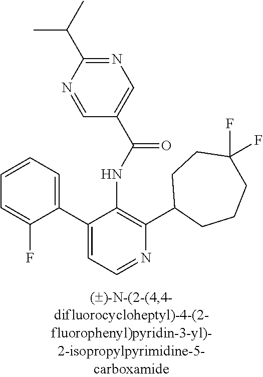 [SMILES: CC(C)c1ncc(C(=O)Nc2c(-c3ccccc3F)ccnc2C2CCCC(F)(F)CC2)cn1] | 0.083 g (60%) | 469.8 (M + 1) | (400 MHz, DMSO-d₆) δ 10.31 (s, 1H), 8.92 (s, 2H), 8.60 (d, J = 4.9 Hz, 1H), 7.44-7.21 (m, 5H), 3.32-3.26 (m, 1H), 3.19 (sept, J = 6.9 Hz, 1H), 2.33-2.20 (m, 1H), 2.20-2.02 (m, 3H), 1.94-1.76 (m, 5H), 1.60-1.49 (m, 1H), 1.28 (d, J = 6.9 Hz, 6H); (376 MHz, DMSO-d₆) δ −82.5 (d, J = 237 Hz), −83.9 (d, J = 237 Hz), −114.9 |
| 38 | 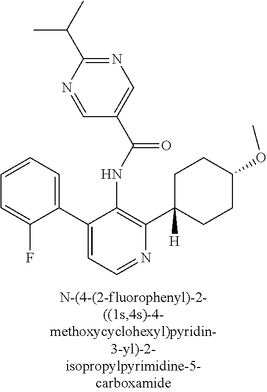 [SMILES: CO[C@H]1CC[C@@H](c2nccc(-c3ccccc3F)c2NC(=O)c2cnc(C(C)C)nc2)CC1] | 0.031 g (13%) | 449.2 (M + 1) | (400 MHz, DMSO-d₆) δ 10.29 (s, 1H), 8.92 (s, 2H), 8.59 (d, J = 4.9 Hz, 1H), 7.43-7.34 (m, 2H), 7.32-7.20 (m, 3H), 3.43 (s, 1H), 3.24 (s, 3H), 3.18 (sept, J = 6.9 Hz, 1H), 3.07-3.00 (m, 1H), 1.96-1.93 (m, 4H), 1.50-1.38 (m, 4H), 1.28 (d, J = 6.9 Hz, 6H); (376 MHz, DMSO-d₆) δ −114.9 |
| 39 | 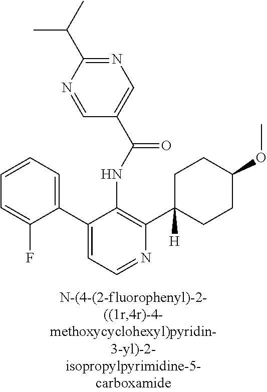 [SMILES: CO[C@H]1CC[C@H](c2nccc(-c3ccccc3F)c2NC(=O)c2cnc(C(C)C)nc2)CC1] | 0.050 g (21%) | 449.2 (M + 1) | (400 MHz, DMSO-d₆) δ 10.30 (s, 1H), 8.91 (s, 2H), 8.58 (d, J = 4.9 Hz, 1H), 7.43-7.33 (m, 2H), 7.32-7.26 (m, 2H), 7.22 (td, J = 7.5, 1.0 Hz, 1H), 3.24-3.22 (m, 3H), 3.22-3.13 (m, 2H), 2.98 (tt, J = 11.5, 3.4 Hz, 1H), 2.10-2.06 (m, 2H), 1.82-1.64 (m, 4H), 1.28 (d, J = 6.9 Hz, 6H), 1.24-1.13 (m, 2H); (376 MHz, DMSO-d₆) δ −115.0 |

### Example 40

### Preparation of 2-isopropyl-N-(4-phenyl-2-(pyrrolidin-2-yl)pyridin-3-yl)pyrimidine-5-carboxamide

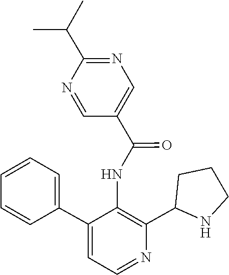 [SMILES: CC(C)c1ncc(C(=O)Nc2c(-c3ccccc3)ccnc2C2CCCN2)cn1]

To a mixture of (±)-tert-butyl 2-(3-(2-isopropylpyrimidine-5-carboxamido)-4-phenylpyridin-2-yl)pyrrolidine-1-carboxylate (0.054 g, 0.11 mmol) in dichloromethane (0.4 mL) was added trifluoroacetic acid (0.20 mL, 2.6 mmol). The reaction mixture was stirred at ambient temperature for 1 h, and the volatiles were removed in vacuo. The residue was purified by reverse-phase column chromatography, using a gradient of 0 to 100% acetonitrile in water containing 0.5% formic acid as eluent, to afford the title compound as a colorless solid (0.027 g, 43% yield): ¹H NMR (400 MHz, DMSO-d₆) δ 9.04 (s, 2H), 8.63-8.62 (m, 1H), 8.32 (s, 1H), 7.50-7.35 (m, 6H), 4.68-4.64 (m, 1H), 3.27-3.13 (m, 2H), 3.01-2.95 (m, 1H), 1.88-1.73 (m, 4H), 1.28 (d, J=6.7 Hz, 6H); MS (ES+) m/z 388.2 (M+1).

### Example 41

### Synthesis of (±)-N-[2-(4,4-difluoro-2-piperidyl)-4-(2-fluorophenyl)-3-pyridyl]-2-isopropyl-pyrimidine-5-carboxamide hydrochloric acid salt

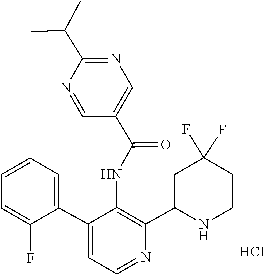

To a mixture of (±)-tert-butyl 4,4-difluoro-2-(4-(2-fluorophenyl)-3-(2-isopropylpyrimidine-5-carboxamido)pyridin-2-yl)piperidine-1-carboxylate (0.174 g, 0.312 mmol) in methanol (3.1 mL) was added hydrochloric acid (3.1 mL, 12 mmol, 4 M in 1,4-dioxane). The reaction mixture was stirred at ambient temperature for 3 h, and concentrated in vacuo to afford the title compound as a colorless solid (0.142 g, 100% yield). Further purification of the title compound (0.020 g) by reverse-phase column chromatography, using a gradient of 5 to 100% acetonitrile in water containing 0.5% formic acid as eluent, afforded a colorless solid. The residue was re-dissolved in anhydrous 1,4-dioxane (0.3 mL), and hydrochloric acid (0.10 mL, 0.38 mmol, 4 M in dioxane) was added at ambient temperature. The mixture was stirred at ambient temperature for 5 minutes, diluted in diethyl ether (2 mL), and the mixture was filtered to afford the title compound as a colorless solid (0.003 g): ¹H NMR (400 MHz, DMSO-d₆) δ 10.76 (s, 1H), 9.89-9.86 (m, 1H), 9.44 (q, J=10.0 Hz, 1H), 9.07-8.99 (m, 2H), 8.81 (d, J=5.0 Hz, 1H), 7.70 (dd, J=5.0, 1.0 Hz, 1H), 7.51-7.41 (m, 2H), 7.37-7.27 (m, 2H), 4.90 (t, J=10.4 Hz, 1H), 3.55-3.46 (m, 1H), 3.34-3.27 (m, 1H), 3.21 (sept, J=6.9 Hz, 1H), 2.89-2.72 (m, 1H), 2.43-2.22 (m, 3H), 1.29 (d, J=6.9 Hz, 6H); MS (ES+) m/z 456.2 (M+1).

### Example 42

### Synthesis of (±)-N-(4-(2-fluorophenyl)-2-(1-methylpyrrolidin-2-yl)pyridin-3-yl)-2-isopropylpyrimidine-5-carboxamide

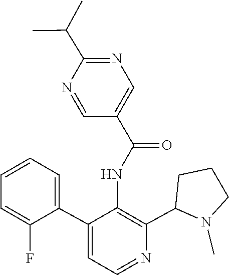 [SMILES: CC(C)c1ncc(C(=O)Nc2c(-c3ccccc3F)ccnc2C2CCCN2C)cn1]

To a mixture of (±)-tert-butyl 2-(3-(2-isopropylpyrimidine-5-carboxamido)-4-phenylpyridin-2-yl)pyrrolidine-1-carboxylate (0.415 g, 0.938 mmol) in N,N,-dimethylformamide (9.4 mL) at 0° C. was added triethylamine (0.82 mL, 4.7 mmol), iodomethane (0.49 mL, 1.126 mmol), and the reaction mixture was stirred at ambient temperature for 2 days. The mixture was diluted with ethyl acetate (150 mL), washed with saturated ammonium chloride (50 mL), water (3×50 mL), dried with magnesium sulfate, filtered and concentrated in vacuo. The residue was purified by column chromatography, eluting with a gradient of 30 to 100% ethyl acetate in heptane, then 0 to 25% methanol in ethyl acetate, to afford a colorless solid (0.077 g, 19% yield): ¹H NMR (400 MHz, DMSO-d₆) δ 10.91 (s, 1H), 8.94 (s, 2H), 8.57 (d, J=4.9 Hz, 1H), 7.44-7.35 (m, 3H), 7.29-7.18 (m, 2H), 3.71-3.67 (m, 1H), 3.24-3.09 (m, 2H), 2.31 (quintet, J=8.3 Hz, 1H), 2.16-2.10 (m, 4H), 1.92-1.83 (m, 1H), 1.80-1.66 (m, 2H), 1.28 (d, J=6.9 Hz, 6H); MS (ES+) m/z 420.2 (M+1).

### Example 43

In a similar manner as described in EXAMPLE 42, utilizing the appropriately substituted starting materials and intermediates, the following compound was prepared:

| | | Amount (g) | |
|---|---|---|---|
| Example | Structure | Yield % | |
| No. | Name | MS (ES+) m/z | ¹H NMR |
| 43 | 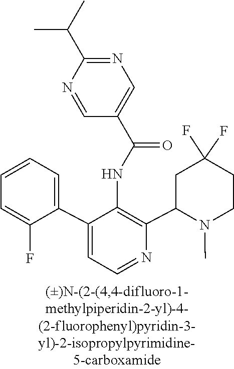 [SMILES: CC(C)c1ncc(C(=O)Nc2c(-c3ccccc3F)ccnc2C2CC(F)(F)CCN2C)cn1] | 0.046 g 32% 470.2 (M + 1) | (400 MHz, DMSO d₆) δ 10.50 (s, 1H), 8.92 (s, 2H), 8.67 (d, J = 4.8 Hz, 1H), 7.45-7.36 (m, 3H), 7.29 (t, J = 9.3 Hz, 1H), 7.24 (t, J = 7.5 Hz, 1H), 3.65 (d, J = 10.1 Hz, 1H), 3.19 (sept, J = 6.9 Hz, 1H), 2.97 (d, J = 9.8 Hz, 1H), 2.47-2.26 (m, 2H), 2.16-2.02 (m, 3H), 1.92 (s, 3H), 1.28 (d, J = 6.9 Hz, 6H) |

### Example 44

### Synthesis of 2-(2-(3,6-dihydro-2H-pyran-4-yl)-4-phenylpyridin-3-yl)-3,4,6,7-tetrahydropyrano[3,4-d]imidazole

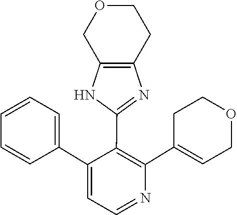 [SMILES: C1=C(c2nccc(-c3ccccc3)c2-c2nc3c([nH]2)COCC3)CCOC1]

### Step 1. Preparation of 2-chloro-4-phenylnicotinaldehyde

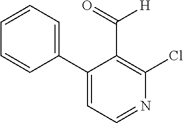 [SMILES: O=Cc1c(-c2ccccc2)ccnc1Cl]

To a mixture of 2-chloro-4-iodonicotinaldehyde (5.0 g, 18.7 mmol) in anhydrous dioxane (63 mL) and water (7 mL) was added phenylboronic acid (2.5 g, 21 mmol) and potassium carbonate (6.5 g, 47 mmol). The mixture was purged with nitrogen for 10 minutes. To the mixture was then added dichloro[1,1′-bis(diphenyl-phosphino)ferrocene]palladium(II) dichloromethane adduct (1.5 g, 1.9 mmol) and the reaction mixture was heated to 90° C. for 3 h. After cooling to ambient temperature, the reaction mixture was filtered through a bed of diatomaceous earth (i.e., Celite®) and the filtrate concentrated under reduced pressure. Purification of the residue by column chromatography, eluting with a gradient of 0 to 30% of ethyl acetate in heptane, afforded the title compound as an off-white solid (4.0 g, 99% yield): ¹H-NMR (300 MHz; DMSO-d₆) δ 10.11 (s, 1H), 8.62 (d, J=5.1 Hz, 1H), 7.57 (d, J=5.1 Hz, 1H), 7.53-7.50 (m, 3H), 7.46-7.43 (m, 2H).

### Step 2. Preparation of 2-(2-chloro-4-phenylpyridin-3-yl)-3,4,6,7-tetrahydropyrano[3,4-d]imidazole

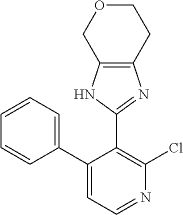 [SMILES: Clc1nccc(-c2ccccc2)c1-c1nc2c([nH]1)COCC2]

To a solution of 2-chloro-4-phenylnicotinaldehyde (0.60 g, 2.8 mmol), 3-bromooxan-4-one (0.74 g, 4.1 mmol) and concentrated ammonium hydroxide (0.65 mL, 4.1 mmol) in anhydrous N,N-dimethylformamide (5.6 mL) was added ammonium acetate (0.96 g, 12 mmol) in portions. The reaction mixture was stirred at ambient temperature for 45 minutes, and oxone (0.16 g, 2.7 mmol) was added to it. The reaction mixture was stirred at 65° C. for another 24 h. After cooling to ambient temperature, the reaction mixture was diluted with ethyl acetate (30 mL) and extracted with water (30 mL×3). The combined organic layers were washed with brine (50 mL), dried over anhydrous sodium sulfate, filtered and concentrated in vacuo. Purification of the residue by column chromatography, eluting with a gradient of 20-100% of ethyl acetate in heptane then 0-10% of methanol in dichloromethane, to afford the title compound as a yellow solid (0.27 g, 31% yield): ¹H-NMR (300 MHz; MeOD): δ 8.46 (d, J=5.2 Hz, 1H), 7.49 (d, J=5.2 Hz, 1H), 7.31-7.28 (m, 3H), 7.19 (d, J=1.8 Hz, 2H), 4.54 (t, J=1.5 Hz, 2H), 3.90 (t, J=5.5 Hz, 2H), 2.62 (ddd, J=5.5, 3.9, 1.5 Hz, 2H); MS (ESI+) m/z=312.2 (M+1); 314.0 (M+1).

### Step 3. Preparation of 2-(2-(3,6-dihydro-2H-pyran-4-yl)-4-phenylpyridin-3-yl)-3,4,6,7-tetrahydropyrano[3,4-d]imidazole

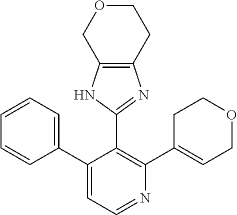 [SMILES: C1=C(c2nccc(-c3ccccc3)c2-c2nc3c([nH]2)COCC3)CCOC1]

A mixture of 2-(2-chloro-4-phenylpyridin-3-yl)-3,4,6,7-tetrahydropyrano[3,4-d]imidazole (0.10 g, 0.32 mmol), 3,6-dihydro-2H-pyran-4-boronic acid pinacol ester (0.20 g, 0.96 mmol) and palladium(II) acetate (0.021 g, 0.096 mmol), tricyclohexylphosphine tetrafluoroborate (0.071 g, 0.19 mmol) and potassium phosphate tribasic (0.48 g, 2.2 mmol) in 1,4-dioxane (3.2 mL) was stirred for 15 min under a nitrogen atmosphere. To this mixture was added water (0.80 mL) and it was stirred at 110° C. for 12 h. The mixture was filtered through diatomaceous earth (i.e., Celite®) and the filtrate was concentrated in vacuo. Purification by reverse phase preparative HPLC, using 15-95% acetonitrile in water containing 0.5% formic acid as eluent, afforded the title compound (0.012 g, 12% yield): ¹H-NMR (300 MHz; DMSO-d₆): δ 8.65 (d, J=5.0 Hz, 1H), 8.16 (s, 1H), 7.37 (d, J=5.0 Hz, 1H), 7.31 (m, 3H), 7.19 (m, 2H), 5.61 (s, 1H), 4.44 (s, 2H), 4.01 (m, 2H), 3.82-3.78 (m, 2H), 3.66 (t, J=5.3 Hz, 2H), 2.54 (m, 2H), 2.18 (m, 2H); MS (ESI+) m/z 360.2 (M+1).

### Example 45

### Synthesis of 2-(4-phenyl-2-(tetrahydro-2H-pyran-4-yl)pyridin-3-yl)-3,4,6,7-tetrahydropyrano[3,4-d]imidazole

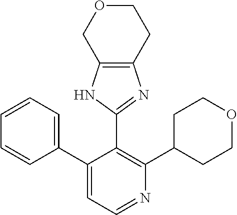 [SMILES: c1ccc(-c2ccnc(C3CCOCC3)c2-c2nc3c([nH]2)COCC3)cc1]

To a stirred solution of 2-(2-(3,6-dihydro-2H-pyran-4-yl)-4-phenylpyridin-3-yl)-3,4,6,7-tetrahydropyrano[3,4-d]imidazole (0.10 g, 0.28 mmol) in methanol (1.4 mL) and ethyl acetate (1.4 mL) under a nitrogen atmosphere was added palladium on carbon (10%, wet support) (0.044 g, 0.42 mmol) and ammonium formate (0.63 g, 10 mmol). The reaction mixture was heated to 65° C. for 1 h. To the mixture was added palladium on carbon (10%, wet support) (0.022 g, 0.21 mmol) and ammonium formate (0.32 g, 5 mmol). The mixture was stirred at 65° C. for 20 h. After cooling to ambient temperature, the mixture was diluted with ethyl acetate (20 mL) and washed with water (2×25 mL). The organic phase was dried over anhydrous magnesium sulfate, filtered, and concentrated in vacuo. Purification of the residue by preparative reverse phase HPLC, using 15-95% acetonitrile in water containing 0.5% of formic acid as eluent, afforded the title compound as a colorless solid (0.0055 g, 5.4% yield). ¹H-NMR (300 MHz; DMSO-d₆): δ 11.66 (s, 1H), 8.66 (d, J=5.1 Hz, 1H), 7.33 (d, J=5.1 Hz, 1H), 7.32-7.29 (m, 3H), 7.20-7.15 (m, 2H), 4.50 (s, 2H), 3.91-3.85 (m, 2H), 3.83-3.80 (m, 2H), 2.96-2.75 (m, 2H), 2.03-1.83 (m, 3H), 1.62-1.56 (m, 2H), 1.29-1.17 (m, 2H); MS (ESI+) m/z 362.2 (M+1).

### Example 46

### Synthesis of 2-(2-(4,4-difluorocyclohexyl)-4-phenylpyridin-3-yl)-3,4,6,7-tetrahydropyrano[3,4-d]imidazole

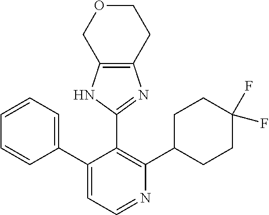 [SMILES: FC1(F)CCC(c2nccc(-c3ccccc3)c2-c2nc3c([nH]2)COCC3)CC1]

To a mixture of (4,4″-di-t-butyl-2,2″-bipyridine)bis[3,5-difluoro-2-[5-trifluoromethyl-2-pyridinyl-KN)phenyl-KC]iridium(III) hexafluorophosphate (2.9 mg, 0.0026 mmol), NiCl₂.glyme (5.6 mg, 0.026 mmol), 4,4′-di-tert-butyl-2,2′-bipyridine (10.3 mg, 0.039 mmol), 2-(2-chloro-4-phenylpyridin-3-yl)-3,4,6,7-tetrahydropyrano[3,4-d]imidazole (0.080 g, 0.26 mmol), cesium carbonate (0.13 g, 0.39 mmol) and 4,4-difluorocyclohexanecarboxylic acid (0.064 g, 0.39 mmol, 1.5 equiv) was added N,N-dimethylformamide (5.8 mL). The mixture was purged with nitrogen for 10 s and sealed. The reaction mixture was then stirred in front of Kessil PR160L lights (440 nm) for 20 h. The reaction mixture was quenched with saturated aqueous sodium bicarbonate solution (10 mL) and extracted with ethyl acetate (3×10 mL). The combined organic extracts were washed with water (3×25 mL) and brine (50 mL), dried over anhydrous sodium sulfate, and concentrated in vacuo. Purification of the residue by preparative reverse phase HPLC, using 5-50% acetonitrile in water containing 0.5% of formic acid as eluent, afforded the title compound as a colorless solid (0.012 g, 12% yield). ¹H-NMR (400 MHz; DMSO-d₆) δ 11.60 (s, 1H), 8.65 (d, J=5.0 Hz, 1H), 7.35 (d, J=5.0 Hz, 1H), 7.31 (m, 3H), 7.18-7.16 (m, 2H), 4.52-4.45 (m, 2H), 3.82 (s, 2H), 2.78-2.70 (m, 1H), 2.11-1.63 (m, 9H). MS (ESI+) m/z 396.2 (M+1).

### Example 47

### Synthesis of 2-(2-(4,4-difluorocyclohexyl)-4-(2,5-difluorophenyl)pyridin-3-yl)-3,4,6,7-tetrahydropyrano[3,4-d]imidazole

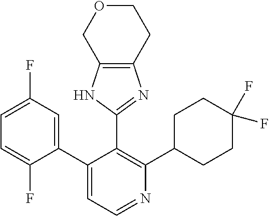 [SMILES: Fc1ccc(F)c(-c2ccnc(C3CCC(F)(F)CC3)c2-c2nc3c([nH]2)COCC3)c1]

### Step 1: Preparation of 2-chloro-4-(2,5-difluorophenyl)nicotinaldehyde

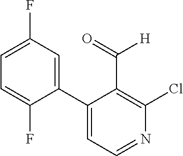 [SMILES: O=Cc1c(-c2cc(F)ccc2F)ccnc1Cl]

To a solution of 2,5-difluorophenylboronic acid (3.3 g, 21 mmol), 2-chloro-4-iodonicotinaldehyde (5.0 g, 19 mmol), [1,1′-bis(diphenylphosphino)ferrocene]dichloropalladium(II), complex with dichloromethane (1:1) (1.6 g, 1.9 mmol) in 1,4-dioxane (67 mL) was added potassium carbonate (7.8 g, 56 mmol) and purged with nitrogen for 5 min. To this was added water (7.5 mL) and the mixture was stirred for 3 h at 90° C. After cooling to ambient temperature, the reaction mixture was filtered through a bed of diatomaceous earth (i.e., Celite®) and the filtrate concentrated under reduced pressure. Purification of the residue by column chromatography, eluting with a gradient of 0 to 40% of ethyl acetate in heptane, afforded the title compound as an off-white solid (4.2 g, 87% yield): MS (ESI+) m/z 254.0 (M+1) 256.0 (M+1).

### Step 2. Preparation of 2-(2-chloro-4-(2,5-difluorophenyl)pyridin-3-yl)-3,4,6,7-tetrahydropyrano[3,4-d]imidazole

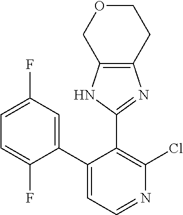 [SMILES: Fc1ccc(F)c(-c2ccnc(Cl)c2-c2nc3c([nH]2)COCC3)c1]

To a solution of 2-chloro-4-(2,5-difluorophenyl)nicotinaldehyde (0.30 g, 1.2 mmol), 3-bromooxan-4-one (0.42 g, 2.4 mmol) and concentrated ammonium hydroxide (0.64 mL, 16 mmol) in anhydrous N,N-dimethylformamide (2.4 mL) was added ammonium acetate (0.41 g, 5.3 mmol). The reaction mixture was stirred at ambient temperature for 45 minutes and then oxone (0.36 g, 0.59 mmol) was added to it. The reaction mixture was stirred at 65° C. for another 12 h. After cooling to ambient temperature, the reaction mixture was diluted with ethyl acetate (20 mL) and washed with water (20 mL×3). The combined organic layers were washed with brine (40 mL), dried over anhydrous sodium sulfate, filtered and concentrated in vacuo. Purification of the residue by column chromatography, eluting with a gradient of 55-100% of ethyl acetate in heptane, afforded the title compound as a yellow solid (0.20 g, 49% yield): MS (ESI+) m/z=348.0 (M+1), 350.2 (M+1).

### Step 3. Preparation of 2-(2-(4,4-difluorocyclohexyl)-4-(2,5-difluorophenyl)pyridin-3-yl)-3,4,6,7-tetrahydropyrano[3,4-d]imidazole

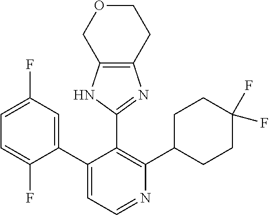 [SMILES: Fc1ccc(F)c(-c2ccnc(C3CCC(F)(F)CC3)c2-c2nc3c([nH]2)COCC3)c1]

To a mixture of (4,4″-di-t-butyl-2,2″-bipyridine)bis[3,5-difluoro-2-[5-trifluoromethyl-2-pyridinyl-KN)phenyl-KC]iridium(III) hexafluorophosphate (2.9 mg, 0.0026 mmol), NiCl₂.glyme (5.6 mg, 0.026 mmol), 4,4′-di-tert-butyl-2,2′-bipyridine (10.3 mg, 0.039 mmol), 2-(2-chloro-4-phenylpyridin-3-yl)-3,4,6,7-tetrahydropyrano[3,4-d]imidazole (0.080 g, 0.26 mmol), cesium carbonate (0.13 g, 0.39 mmol), and 4,4-difluorocyclohexanecarboxylic acid (0.056 g, 0.35 mmol) was added N,N-dimethylformamide (5.8 mL). The reaction mixture was purged with nitrogen for 10s and sealed with a cap. The reaction mixture was stirred in front of Kessil PR160L lights (440 nm) for 20 h. The reaction mixture was quenched with saturated aqueous sodium bicarbonate solution (10 mL) and extracted with ethyl acetate (3×10 mL). The combined organic extracts were washed with water (3×25 mL) and brine (50 mL), dried over anhydrous sodium sulfate, and concentrated in vacuo. Purification of the residue by preparative reverse phase HPLC, using 5-50% acetonitrile in water containing 0.5% of formic acid as eluent, afforded the title compound as a colorless solid (0.0023 g, 2.2% yield); ¹H-NMR (400 MHz; MeOD) δ 8.69 (d, J=4.6 Hz, 1H), 8.39 (s, 1H), 7.37 (d, J=4.2 Hz, 1H), 7.12-7.10 (m, 2H), 6.93-6.90 (m, 1H), 4.59 (s, 2H), 3.94-3.93 (m, 2H), 2.66 (dd, J=3.8, 0.7 Hz, 2H), 2.14-2.02 (m, 4H), 1.90-1.85 (m, 2H), 1.81-1.65 (m, 3H); MS (ESI+) m/z 432.2 (M+1).

### Example 48

### Synthesis of 2-(2-(3,3-difluorocyclopentyl)-4-(2,5-difluorophenyl)pyridin-3-yl)-3,4,6,7-tetrahydropyrano[3,4-d]imidazole

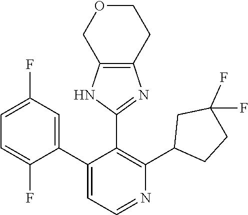 [SMILES: Fc1ccc(F)c(-c2ccnc(C3CCC(F)(F)C3)c2-c2nc3c([nH]2)COCC3)c1]

A 20 mL vial was charged with (4,4″-di-t-butyl-2,2″-bipyridine)bis[3,5-difluoro-2-[5-trifluoromethyl-2-pyridinyl-KN)phenyl-KC]iridium(III) hexafluorophosphate (2.9 mg, 0.0026 mmol), nickel(II) chloride ethylene glycol dimethyl ether complex (5.6 mg, 0.026 mmol), 4,4′-di-tert-butyl-2,2′-bipyridine (10.3 mg, 0.039 mmol), 2-(2-chloro-4-phenylpyridin-3-yl)-3,4,6,7-tetrahydropyrano[3,4-d]imidazole (0.080 g, 0.26 mmol), cesium carbonate (0.13 g, 0.39 mmol), 3,3-difluorocyclopentanecarboxylic acid (0.052 g, 0.35 mmol), a magnetic stirring bar, and DMF (5.8 mL). The reaction mixture was purged with nitrogen for 10 s and sealed with a cap. The reaction mixture was then stirred in front of Kessil PR160L lights (440 nm) for 20 h. The reaction mixture was quenched with saturated aqueous sodium bicarbonate solution (10 mL) and extracted with ethyl acetate (3×10 mL). The combined organic extracts were washed with water (3×25 mL) and brine (50 mL), dried over anhydrous sodium sulfate, and concentrated in vacuo. Purification of the residue by preparative reverse phase HPLC, using 5-50% acetonitrile in water containing 0.5% of formic acid as eluent, afforded the title compound as an off-white solid (0.0025 g, 2.6% yield): ¹H-NMR (400 MHz; MeOD) δ 8.74 (d, J=5.0 Hz, 1H), 8.27 (s, 1H), 7.38 (dd, J=5.0, 0.9 Hz, 1H), 7.16-7.07 (m, 2H), 6.92 (ddd, J=9.0, 5.2, 2.4 Hz, 1H), 4.59 (s, 2H), 3.95 (t, J=5.5 Hz, 2H), 3.46-3.36 (m, 1H), 2.68-2.66 (m, 2H), 2.63-2.50 (m, 1H), 2.38-2.27 (m, 2H), 2.15-1.99 (m, 3H); MS (ESI+) m/z 418.2 (M+1).

### Example 49

### Synthesis of N-(6-chloropyridin-3-yl)-2-cyclopentyl-4-(2-fluorophenyl)nicotinamide

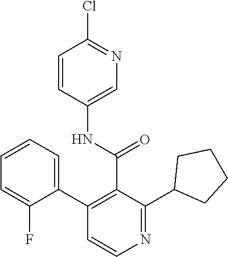 [SMILES: O=C(Nc1ccc(Cl)nc1)c1c(-c2ccccc2F)ccnc1C1CCCC1]

### Step 1. Preparation of 2-chloro-4-iodonicotinic acid

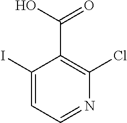 [SMILES: O=C(O)c1c(I)ccnc1Cl]

To a mixture of 2-chloro-4-iodopyridine (38.00 g, 158.7 mmol) in tetrahydrofuran (190 mL) was added 2 M lithium diisopropylamide (87.0 mL, 175 mmol) at −70° C. under an atmosphere of nitrogen and stirred for 1 h. Solid carbon dioxide (209.5 g, 4760 mmol) was added the reaction mixture was stirred at −70° C. for 2 h. After warming to ambient temperature, the mixture was diluted with 12 M hydrochloric acid until pH 2 was obtained. The mixture was extracted with 50% ethyl acetate in tetrahydrofuran (3×400 mL). The combined organic solution was dried over anhydrous sodium sulfate, filtered and concentrated in vacuo. Purification of the residue by trituration with 9% ethyl acetate in petroleum ether (110 mL) and filtration afforded the title compound as a yellow solid (31 g, 69% yield): ¹H NMR (400 MHz, DMSO-d₆) δ 14.3 (br s, 1H), 8.12 (d, J=4.8, 1H), 7.98 (d, J=5.2, 1H).

### Step 2. Preparation of tert-butyl 2-chloro-4-iodonicotinate

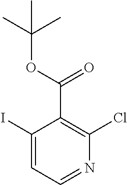

To a mixture of 2-chloro-4-iodonicotinic acid (10.00 g, 35.28 mmol) in tert-butanol (100 mL) was added 4-(dimethylamino)pyridine (0.431 g, 3.53 mmol), triethylamine (3.57 g, 35.3 mmol), and di-tert-butyl dicarbonate (15.40 g, 70.56 mmol) and purged with nitrogen. The reaction mixture was stirred at 50° C. for 12 h. After cooling to ambient temperature, the mixture was concentrated in vacuo. Purification of the residue column chromatography, using 17% ethyl acetate in petroleum ether as eluent, afforded the title compound as a yellow solid (31 g, 69% yield): MS (ES+) m/z 339.9 (M+1) and 441.9 (M+1).

### Step 3. Preparation of tert-butyl 2-chloro-4-(2-fluorophenyl)nicotinate

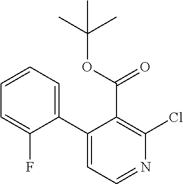 [SMILES: CC(C)(C)OC(=O)c1c(-c2ccccc2F)ccnc1Cl]

To a mixture of tert-butyl 2-chloro-4-iodonicotinate (8.50 g, 25.0 mmol) in dioxane (44 mL) and water (11 mL) was added (2-fluorophenyl)boronic acid (4.20 g, 30.0 mmol), potassium carbonate (6.92 g, 35.3 mmol), and bis(diphenylphosphino)ferrocene palladium (II) dichloromethane (1.83 g, 2.50 mmol) and purged with nitrogen. The reaction mixture was stirred at 80° C. for 12 h. After cooling to ambient temperature, the mixture was diluted with water (250 mL) and extracted with ethyl acetate (3×200 mL). The combined organic solution was dried over anhydrous sodium sulfate, filtered and concentrated in vacuo. Purification of the residue by column chromatography, using 5% ethyl acetate in petroleum ether as eluent, afforded the title compound as a colorless oil (7.00 g, 91% yield): MS (ES+) m/z 308.0 (M+1) and 310.0 (M+1).

### Step 4: Preparation of tert-butyl 2-(cyclopent-1-en-1-yl)-4-(2-fluorophenyl)nicotinate

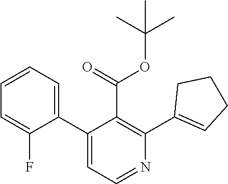 [SMILES: CC(C)(C)OC(=O)c1c(-c2ccccc2F)ccnc1C1=CCCC1]

To a mixture of tert-butyl 2-chloro-4-(2-fluorophenyl)nicotinate (1.00 g, 3.25 mmol), 2-(cyclopent-1-en-1-yl)-4,4,5,5-tetramethyl-1,3,2-dioxaborolane (0.946 g, 4.87 mmol) and sodium carbonate (1.55 g, 14.6 mmol) in 1,4-dioxane (8 mL) and water (0.8 mL) was added [1,1′-bis(diphenylphosphino)ferrocene]-dichloropalladium(II) (0.238 g, 0.325 mmol) in glove box. The mixture was stirred at 120° C. for 2 h under microwave irradiation. After the mixture was cooled to ambient temperature, thiourea resin (0.100 g) was added. The mixture was stirred for 4 h. The mixture was filtered and concentrated in vacuo. The residue was purified by column chromatography, eluting with a 10:1 mixture of petroleum ether in ethyl acetate, afford the title compound as a yellow solid (0.700 g, 63% yield): ¹H NMR (400 MHz, CDCl₃) δ 8.62 (d, J=4.8 Hz, 1H), 7.43-7.36 (m, 1H), 7.32-7.27 (m, 1H), 7.22-7.13 (m, 2H), 7.12 (d, J=4.8 Hz, 1H), 6.17 (quin, J=2.0 Hz, 1H), 2.93-2.86 (m, 2H), 2.55 (qt, J=7.2, 2.4 Hz, 2H), 2.09-1.99 (m, 2H), 1.25 (s, 9H).

### Step 5. Preparation of 4-tert-butyl 2-cyclopentyl-4-(2-fluorophenyl)-nicotinate

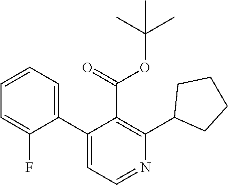 [SMILES: CC(C)(C)OC(=O)c1c(-c2ccccc2F)ccnc1C1CCCC1]

To a solution of tert-butyl 2-(cyclopent-1-en-1-yl)-4-(2-fluorophenyl)-nicotinate (0.500 g, 1.47 mmol) in methanol (10 mL) was added palladium on carbon (0.300 g, 10% purity) under a nitrogen atmosphere. The suspension was degassed in vacuo and purged with hydrogen several times. The mixture was stirred under hydrogen (15 psi) at 25° C. for 12 h. The mixture was filtered. The filtrate was concentrated in vacuo. The residue was directly used in the next step without further purification (0.500 g, crude): ¹H NMR (400 MHz, CDCl₃) δ 8.62 (d, J=4.8 Hz, 1H), 7.43-7.36 (m, 1H), 7.33-7.28 (m, 1H), 7.22-7.13 (m, 2H), 7.09 (d, J=4.8 Hz, 1H), 3.39 (quin, J=8.0 Hz, 1H), 2.10-1.95 (m, 4H), 1.93-1.85 (m, 2H), 1.72-1.66 (m, 2H), 1.28 (s, 9H).

### Step 6. Preparation of 2-cyclopentyl-4-(2-fluorophenyl)nicotinic acid

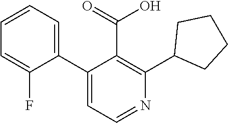 [SMILES: O=C(O)c1c(-c2ccccc2F)ccnc1C1CCCC1]

To a solution of tert-butyl 2-cyclopentyl-4-(2-fluorophenyl)nicotinate (0.500 g, 1.46 mmol) in dichloromethane (5 mL) was added trifluoroacetic acid (3.85 g, 33.8 mmol) at 0° C. The mixture was stirred at 25° C. for 12 h. The mixture was concentrated in vacuo. The mixture was purified by preparative HPLC, eluting with a gradient of 17-47% acetonitrile in 0.225% formic acid in water, to afford the title compound as a colorless solid (0.240 g, 57% yield): ¹H NMR (400 MHz, CDCl₃) δ 8.68 (d, J=5.2 Hz, 1H), 7.44-7.33 (m, 2H), 7.23-7.12 (m, 3H), 3.49-3.38 (m, 1H), 2.13-2.02 (m, 2H), 1.99-1.83 (m, 4H), 1.75-1.62 (m, 2H); MS (ES+) m/z 286.1 (M+1).

### Step 7. Preparation of N-(6-chloropyridin-3-yl)-2-cyclopentyl-4-(2-fluorophenyl)nicotinamide

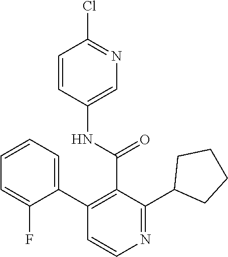 [SMILES: O=C(Nc1ccc(Cl)nc1)c1c(-c2ccccc2F)ccnc1C1CCCC1]

To a mixture of 2-cyclopentyl-4-(2-fluorophenyl)nicotinic acid (0.240 g, 0.841 mmol) and 6-chloropyridin-3-amine (0.216 g, 1.68 mmol) in tetrahydrofuran (0.8 mL) was added N-ethyl-N-isopropylpropan-2-amine (0.326 g, 2.52 mmol) and 2-chloro-1-methylpyridin-1-ium iodide (0.322 g, 1.26 mmol) under a nitrogen atmosphere. The mixture was stirred at 70° C. for 12 h. After cooling to ambient temperature, the mixture was quenched with water (0.1 mL) and concentrated in vacuo. The mixture was purified by preparative HPLC, eluting with a gradient of 50-72% of acetonitrile in 0.225% formic acid in water, to afford the title compound as a colorless solid (0.0129 g, 4% yield): ¹H NMR (400 MHz, CDCl₃) δ 8.72 (d, J=5.2 Hz, 1H), 8.08 (d, J=2.8 Hz, 1H), 7.96 (dd, J=8.8, 2.8 Hz, 1H), 7.41-7.33 (m, 2H), 7.26-7.20 (m, 2H), 7.20-7.11 (m, 3H), 3.40 (m, J=8.4 Hz, 1H), 2.12-1.97 (m, 4H), 1.95-1.86 (m, 2H), 1.75-1.64 (m, 2H); MS (ES+) m/z 396.0 (M+1), 398.0 (M+1).

### Example 50

### Synthesis of N-(6-chloropyridin-3-yl)-2-(cyclopent-1-en-1-yl)-4-(2-fluorophenyl)nicotinamide

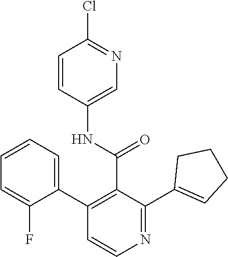 [SMILES: O=C(Nc1ccc(Cl)nc1)c1c(-c2ccccc2F)ccnc1C1=CCCC1]

### Step 1: Preparation of 2-(cyclopent-1-en-1-yl)-4-(2-fluorophenyl)-nicotinic acid

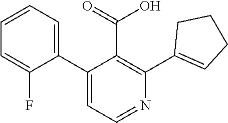 [SMILES: O=C(O)c1c(-c2ccccc2F)ccnc1C1=CCCC1]

To a solution of tert-butyl 2-cyclopentyl-4-(2-fluorophenyl)nicotinate (0.200 g, 0.589 mmol) in dichloromethane (3 mL) was added trifluoroacetic acid (2.31 g, 20.3 mmol) at 0° C. The mixture was stirred at 25° C. for 12 h. The mixture was concentrated in vacuo. The mixture was purified by preparative HPLC, eluting with a gradient of 8-38% of acetonitrile in 0.225% formic acid in water, to afford the title compound as a colorless solid (0.100 g, 59% yield): ¹H NMR (400 MHz, CDCl₃) δ 8.63 (d, J=5.2 Hz, 1H), 7.46-7.39 (m, 1H), 7.35 (dt, J=7.6, 1.8 Hz, 1H), 7.24-7.13 (m, 3H), 6.25 (t, J=2.0 Hz, 1H), 2.92-2.78 (m, 2H), 2.54 (dt, J=7.2, 2.4 Hz, 2H), 2.03 (quin, J=7.6 Hz, 2H); MS (ES+) m/z 284.0 (M+1).

### Step 2. Preparation of N-(6-chloropyridin-3-yl)-2-(cyclopent-1-en-1-yl)-4-(2-fluorophenyl)nicotinamide

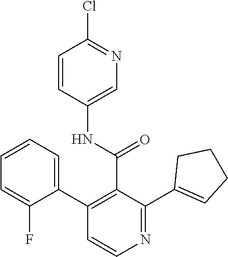 [SMILES: O=C(Nc1ccc(Cl)nc1)c1c(-c2ccccc2F)ccnc1C1=CCCC1]

To a mixture of 2-(cyclopent-1-en-1-yl)-4-(2-fluorophenyl)nicotinic acid (0.0400 g, 0.141 mmol) and 6-chloropyridin-3-amine (0.0547 g, 0.0424 mmol) in tetrahydrofuran (0.4 mL) was added N-ethyl-N-isopropylpropan-2-amine (0.0547 g, 0.424 mmol), 2-chloro-1-methylpyridin-1-ium iodide (0.0541 g, 0.212 mmol) under a nitrogen atmosphere. The mixture was stirred at 70° C. for 12 h. After cooling to ambient temperature, the mixture was quenched with water (0.1 mL) and concentrated in vacuo. The mixture was purified by preparative HPLC, eluting with a gradient of 45-67% of acetonitrile in 0.225% formic acid in water, to afford the title compound as a colorless solid (0.0253 g, 45% yield): ¹H NMR (400 MHz, CDCl₃) δ 8.71 (d, J=4.8 Hz, 1H), 8.13 (d, J=2.4 Hz, 1H), 7.92 (dd, J=2.4, 8.8 Hz, 1H), 7.44-7.32 (m, 3H), 7.26 (s, 1H), 7.25-7.17 (m, 2H), 7.13 (t, J=8.8 Hz, 1H), 6.41 (s, 1H), 2.89 (t, J=6.4 Hz, 2H), 2.58-2.48 (m, 2H), 2.00 (quin, J=7.6 Hz, 2H); MS (ES+) m/z 394.0 (M+1), 396.0 (M+1).

### Example 51

### Synthesis of 4-(2-fluorophenyl)-N-(6-isopropylpyridin-3-yl)-2-(tetrahydro-2H-pyran-4-yl)nicotinamide

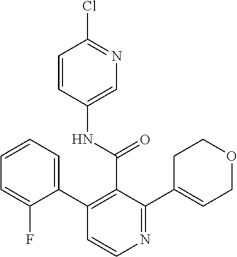 [SMILES: O=C(Nc1ccc(Cl)nc1)c1c(-c2ccccc2F)ccnc1C1=CCOCC1]

### Step 1. Preparation of tert-butyl 2-(3,6-dihydro-2H-pyran-4-yl)-4-(2-fluorophenyl)nicotinate

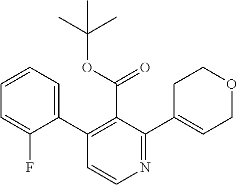 [SMILES: CC(C)(C)OC(=O)c1c(-c2ccccc2F)ccnc1C1=CCOCC1]

To a solution of tert-butyl 2-chloro-4-(2-fluorophenyl)nicotinate (0.400 g, 1.30 mmol) and 2-(3,6-dihydro-2H-pyran-4-yl)-4,4,5,5-tetramethyl-1,3,2-dioxaborolane (0.273 g, 1.30 mmol) in dioxane (8 mL) and water (2 mL) was added sodium carbonate (0.276 g, 2.60 mmol) and [1,1′-bis (diphenylphosphino) ferrocene]dichloropalladium(II) (0.0951 g, 0.130 mmol) under a nitrogen atmosphere. The mixture was stirred at 120° C. for 2 h in microwave. After cooling to ambient temperature, the reaction mixture was concentrated under reduced pressure. The residue was purified by preparative HPLC, eluting with a gradient of 27-47% of acetonitrile in 0.225% formic acid in water, to afford the title compound as a yellow oil (0.368 g, 61% yield): ¹H NMR (400 MHz, CDCl₃) δ 8.89 (d, J=5.6 Hz, 1H), 7.64 (d, J=5.6 Hz, 1H), 7.57-7.49 (m, 1H), 7.38-7.29 (m, 2H), 7.26-7.20 (m, 1H), 6.15 (s, 1H), 4.32 (d, J=2.4 Hz, 2H), 3.98 (t, J=5.2 Hz, 2H), 1.33 (s, 9H).

### Step 2. Preparation of 2-(3,6-dihydro-2H-pyran-4-yl)-4-(2-fluorophenyl) nicotinic acid

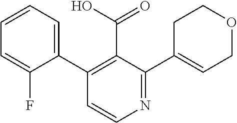 [SMILES: O=C(O)c1c(-c2ccccc2F)ccnc1C1=CCOCC1]

A mixture of tert-butyl 2-(3,6-dihydro-2H-pyran-4-yl)-4-(2-fluorophenyl)nicotinate (0.250 g, 0.703 mmol) in dichloromethane (1.5 mL) and trifluoroacetic acid (1.5 mL) was stirred at 25° C. for 12 h. The reaction mixture was concentrated under reduced pressure. The residue was purified by preparative HPLC, eluting with a gradient of 10-40% of acetonitrile in 0.225% formic acid in water, to afford the title compound as a colorless solid (0.100 g, 47% yield): ¹H NMR (400 MHz, DMSO-d₆) δ 13.66-12.81 (m, 1H), 8.66 (d, J=4.8 Hz, 1H), 7.57-7.45 (m, 1H), 7.43-7.24 (m, 4H), 6.03 (s, 1H), 4.16 (q, J=2.4 Hz, 2H), 3.82 (t, J=5.2 Hz, 2H), 2.61-2.53 (m, 2H); MS (ES+) m/z 300.0 (M+1).

### Step 3. Preparation of 4-(2-fluorophenyl)-N-(6-isopropylpyridin-3-yl)-2-(tetrahydro-2H-pyran-4-yl)nicotinamide

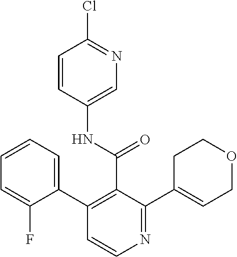 [SMILES: O=C(Nc1ccc(Cl)nc1)c1c(-c2ccccc2F)ccnc1C1=CCOCC1]

To a solution of 2-(3,6-dihydro-2H-pyran-4-yl)-4-(2-fluorophenyl)nicotinic acid (0.0700 g, 0.234 mmol) in tetrahydrofuran (2 mL) were added 2-chloro-1-methylpyridin-1-ium iodide (0.0720 mg, 0.281 mmol), N,N-diisopropylethylamine (0.151 g, 1.17 mmol) and 6-chloropyridin-3-amine (0.0360 g, 0.281 mmol). The mixture was stirred at 70° C. for 36 h. The reaction mixture was concentrated under reduced pressure. The residue was purified by preparative HPLC, eluting with a gradient of 39-59% of acetonitrile in 0.225% formic acid in water, to afford the title compound as a colorless solid (0.00500 g, 50% yield): ¹H NMR (400 MHz, DMSO-d₆) δ 10.81 (s, 1H), 8.73 (d, J=4.8 Hz, 1H), 8.45-8.37 (m, 1H), 7.90-7.84 (m, 1H), 7.48-7.37 (m, 4H), 7.33-7.20 (m, 2H), 6.09 (s, 1H), 4.03 (d, J=2.4 Hz, 2H), 3.76 (t, J=5.2 Hz, 2H), 2.62 (d, J=1.6 Hz, 2H); MS (ES+) m/z 410.0, 412.0 (M+1).

### Example 52

### Synthesis of 4-(2-fluorophenyl)-N-(6-isopropylpyridin-3-yl)-2-(tetrahydro-2H-pyran-4-yl)nicotinamide

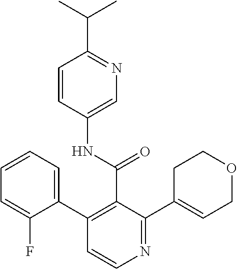 [SMILES: CC(C)c1ccc(NC(=O)c2c(-c3ccccc3F)ccnc2C2=CCOCC2)cn1]

To a solution of 2-(3,6-dihydro-2H-pyran-4-yl)-4-(2-fluorophenyl)nicotinic acid (0.0800 g, 0.267 mmol) in tetrahydrofuran (2 mL) was added 2-chloro-1-methylpyridin-1-ium iodide (0.0820 g, 0.321 mmol), N-ethyl-N-isopropylpropan-2-amine (0.104 g, 0.802 mmol) and 6-isopropylpyridin-3-amine (0.146 g, 1.07 mmol). The mixture was stirred at 70° C. for 12 h. After cooling to ambient temperature, the reaction mixture was concentrated under reduced pressure. The residue was purified by preparative HPLC, eluting with a gradient of 23-43% of acetonitrile in 0.225% formic acid in water, to afford the title compound as a colorless solid (0.00560 g, 0.0112 mmol, 4% yield, 93% purity, formate) as a yellow solid: ¹H NMR (400 MHz, DMSO-d₆) δ 10.56-10.50 (m, 1H), 8.71 (d, J=4.8 Hz, 1H), 8.38 (d, J=2.4 Hz, 1H), 7.69 (dd, J=2.8, 8.4 Hz, 1H), 7.48-7.35 (m, 3H), 7.33-7.15 (m, 3H), 6.15-6.09 (m, 1H), 4.06 (d, J=2.4 Hz, 2H), 3.77 (J=5.2 Hz, 2H), 2.94 (td, J=6.8, 13.6 Hz, 1H), 2.69 (s, 2H), 1.18 (d, J=7.2 Hz, 6H); MS (ES+) m/z 418.1 (M+1).

### Example 53

### Synthesis of 4-(2-fluorophenyl)-N-(6-isopropylpyridin-3-yl)-2-(tetrahydro-2H-pyran-4-yl)nicotinamide

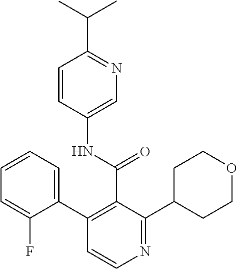 [SMILES: CC(C)c1ccc(NC(=O)c2c(-c3ccccc3F)ccnc2C2CCOCC2)cn1]

### Step 1. Preparation of tert-butyl 4-(2-fluorophenyl)-2-(tetrahydro-2H-pyran-4-yl)nicotinate

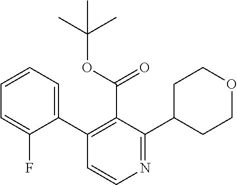 [SMILES: CC(C)(C)OC(=O)c1c(-c2ccccc2F)ccnc1C1CCOCC1]

To a solution of tert-butyl 2-(3,6-dihydro-2H-pyran-4-yl)-4-(2-fluorophenyl)nicotinate (0.450 g, 1.27 mmol) in methanol (5 mL) was added palladium on activated carbon (0.550 g, 0.571 mmol, 10% purity) under a nitrogen atmosphere. The mixture was stirred at 25° C. for 12 h under hydrogen (15 psi). The reaction mixture was filtered and the filtrate was evaporated under reduced pressure to afford the title compound as a colorless solid (0.450 g, 97% yield): ¹H NMR (400 MHz, CDCl₃) δ 8.65 (d, J=5.2 Hz, 1H), 7.45-7.36 (m, 1H), 7.33-7.29 (m, 1H), 7.24-7.11 (m, 3H), 4.12 (dd, J=3.6, 11.2 Hz, 2H), 3.57-3.47 (m, 2H), 3.21 (tt, J=3.6, 11.6 Hz, 1H), 2.16 (dq, J=4.4, 12.6 Hz, 2H), 1.80 (dd, J=1.6, 11.6 Hz, 2H), 1.29 (s, 9H).

### Step 2. Preparation of 4-(2-fluorophenyl)-N-(6-isopropylpyridin-3-yl)-2-(tetrahydro-2H-pyran-4-yl)nicotinamide

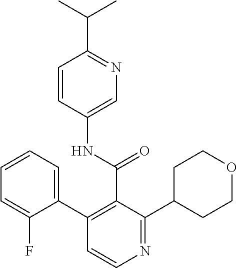 [SMILES: CC(C)c1ccc(NC(=O)c2c(-c3ccccc3F)ccnc2C2CCOCC2)cn1]

To a solution of 4-(2-fluorophenyl)-2-(tetrahydro-2H-pyran-4-yl)nicotinic acid (0.0500 g, 0.166 mmol) in tetrahydrofuran (2 mL) was added 2-chloro-1-methylpyridin-1-ium iodide (0.0510 g, 0.200 mmol), N,N-diisopropylethylamine (0.0640 g, 0.498 mmol) and 6-isopropylpyridin-3-amine (0.0450 g, 0.332 mmol). The mixture was stirred at 70° C. for 12 h. The reaction mixture was concentrated under reduced pressure. The residue was purified by preparative HPLC, eluting with a gradient of 27-47% of acetonitrile in 0.225% formic acid in water, to afford the title compound as a colorless solid (0.00600 g, 7% yield): MS (ES+) m/z 420.0 (M+1).

### Example 54

### Synthesis of N-(2-(4,4-difluorocyclohexyl)-4-(3,4-dihydro-2H-pyran-6-yl)pyridin-3-yl)-2-isopropylpyrimidine-5-carboxamide

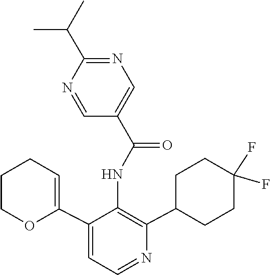 [SMILES: CC(C)c1ncc(C(=O)Nc2c(C3=CCCCO3)ccnc2C2CCC(F)(F)CC2)cn1]

### Step 1. Preparation of tert-butyl (2-chloro-4-iodopyridin-3-yl)carbamate

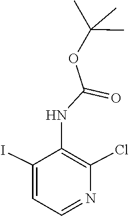 [SMILES: CC(C)(C)OC(=O)Nc1c(I)ccnc1Cl]

To a solution of 2-chloro-4-iodonicotinic acid (5.0 g, 18 mmol) in tert-butanol (84 mL) and N,N-dimethylformamide (71 mL) was added diphenylphosphonic azide (5.7 mL, 26 mmol), and triethylamine (6.1, 44 mmol). The solution was heated at 90° C. for 4 h. The reaction mixture was cooled to ambient temperature and diluted with ethyl acetate (500 mL). The reaction mixture was washed with saturated sodium bicarbonate solution (3×250 mL), water (250 mL), and brine (250 mL). The organic layer was dried over anhydrous magnesium sulfate, filtered and concentrated in vacuo. Purification of the residue by column chromatography, eluting with 5-40% ethyl acetate in heptane, afforded the title compound as an off-white solid (3.4 g, 54% yield): MS (ES+) m/z 355.0 (M+1), 357.0 (M+1).

### Step 2. Preparation of 2-chloro-4-iodopyridin-3-amine trifluoroacetic acid salt

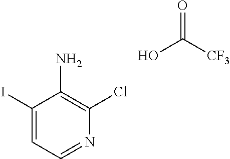 [SMILES: O=C(O)C(F)(F)F ; Nc1c(I)ccnc1Cl]

To a mixture of tert-butyl (2-chloro-4-iodopyridin-3-yl)carbamate (0.82 g, 2.5 mmol) in dichloromethane (25 mL) was added trifluoroacetic acid (15 mL). The reaction mixture was stirred at ambient temperature for 1 h, and the volatiles were removed in vacuo. The light yellow solid was used without further purification (3.4 g, 96% yield): MS (ES+) m/z 254.8 (M+1), 256.8 (M+1).

### Step 3. Preparation of N-(2-chloro-4-iodopyridin-3-yl)-2-isopropylpyrimidine-5-carboxamide

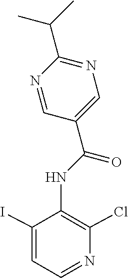 [SMILES: CC(C)c1ncc(C(=O)Nc2c(I)ccnc2Cl)cn1]

To a solution of 2-chloro-4-iodopyridin-3-amine trifluoroacetic acid salt (0.83 g, 3.3 mmol), 2-isopropylpyrimidine-5-carboxylic acid (0.81 g, 4.9 mmol), and 2-chloro-1-methyl-pyridin-1-ium iodide (2.5 g, 9.8 mmol) in tetrahydrofuran (16 mL) was added N,N-diisopropylethylamine (5.7 mL, 33 mmol), and the mixture was stirred at 65° C. for 20 h. After cooling to ambient temperature, the mixture was diluted with ethyl acetate (50 mL), and the organic phase was washed with saturated aqueous sodium bicarbonate (50 mL). The organic phase was dried over anhydrous sodium sulfate, filtered, and concentrated in vacuo. The solution of crude residue was dissolved in methanol/THF (1:1) (18 mL), and sodium hydroxide (16 mL) was then added to the mixture. After stirring for 15 minutes at ambient temperature, the reaction was quenched with saturated ammonium chloride solution (2×30 mL), diluted with ethyl acetate (20 mL), and washed with saturated sodium bicarbonate solution (3×30 mL), water (50 mL), and brine (50 mL). The combined organic layers were dried over anhydrous magnesium sulfate, filtered and concentrated in vacuo. Purification of the residue by column chromatography, eluting with 5-100% ethyl acetate in heptane, afforded the title compound as an off-white solid (0.72 g, 54% yield).

### Step 4. Preparation of N-(2-chloro-4-(3,4-dihydro-2H-pyran-6-yl)pyridin-3-yl)-2-isopropylpyrimidine-5-carboxamide

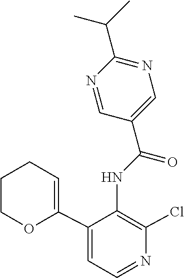 [SMILES: CC(C)c1ncc(C(=O)Nc2c(C3=CCCCO3)ccnc2Cl)cn1]

A mixture of N-(2-chloro-4-iodopyridin-3-yl)-2-isopropylpyrimidine-5-carboxamide (0.090 g, 0.22 mmol) in dioxane (1.2 mL) and water (0.11 mL) was degassed with nitrogen for 10 minutes. To the reaction mixture was added potassium carbonate (0.092 g, 0.67 mmol), 2-(3,4-dihydro-2H-pyran-6-yl)-4,4,5,5-tetramethyl-1,3,2-dioxaborolane (0.070 g, 0.34 mmol), [1,1′-Bis(diphenylphosphino)ferrocene]dichloropalladium(II), complex with CH₂Cl₂ (1:1) (0.019 g, 0.022 mmol), and the reaction mixture was stirred at 90° C. for 3 h. After cooling to ambient temperature, the mixture was filtered through a bed of diatomaceous earth (i.e., Celite®) and the filter pad was washed with ethyl acetate (2×100 mL). The combined filtrate was washed with saturated ammonium chloride (2×75 mL) and concentrated in vacuo. The residue was purified by column chromatography, eluting with a gradient of 10 to 100% ethyl acetate in heptane, to afford the title compound as an off-white solid (0.065 g, 80% yield): MS (ES+) m/z 359.0 (M+1), 361.0 (M+1).

### Step 5. Preparation of N-(2-(4,4-difluorocyclohexyl)-4-(3,4-dihydro-2H-pyran-6-yl)pyridin-3-yl)-2-isopropylpyrimidine-5-carboxamide

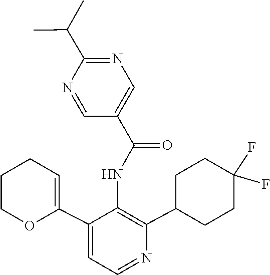 [SMILES: CC(C)c1ncc(C(=O)Nc2c(C3=CCCCO3)ccnc2C2CCC(F)(F)CC2)cn1]

To a mixture of (4,4″-di-t-butyl-2,2″-bipyridine)bis[3,5-difluoro-2-[5-trifluoromethyl-2-pyridinyl-KN)phenyl-KC]iridium(III) hexafluorophosphate (0.0019 g, 0.0017 mmol), NiCl₂.glyme (0.0038 g, 0.017 mmol), 4,4′-di-tert-butyl-2,2′-bipyridine (0.0070 g, 0.026 mmol), N-(2-chloro-4-(3,4-dihydro-2H-pyran-6-yl)pyridin-3-yl)-2-isopropylpyrimidine-5-carboxamide (0.062 g, 0.17 mmol), cesium carbonate (0.11 g, 0.35 mmol) and 4,4-difluorocyclohexanecarboxylic acid (0.056 g, 0.34 mmol) was added N,N-dimethylformamide (4.3 mL). The mixture was purged with nitrogen for 10 s and sealed. The reaction mixture was then stirred in front of Kessil PR160L lights (440 nm) for 20 h. The reaction mixture was quenched with saturated aqueous sodium bicarbonate solution (10 mL) and extracted with ethyl acetate (3×10 mL). The combined organic extracts were washed with water (3×25 mL) and brine (50 mL), dried over anhydrous sodium sulfate, and concentrated in vacuo. Purification of the residue by preparative reverse phase HPLC, using 5-85% acetonitrile in water containing 0.5% of formic acid as eluent, afforded the title compound as a colorless solid (0.010 g, 13% yield): ¹H-NMR (400 MHz; DMSO-d₆) δ 10.21 (s, 1H), 9.21 (s, 2H), 8.49 (d, J=4.9 Hz, 1H), 7.28 (d, J=5.0 Hz, 1H), 5.19 (t, J=3.9 Hz, 1H), 3.96-3.94 (m, 1H), 3.28-3.20 (m, 2H), 3.16-3.08 (m, 1H), 2.10-2.03 (m, 3H), 2.00-1.72 (m, 9H), 1.33 (d, J=6.9 Hz, 6H); MS (ES+) m/z 443.2 (M+1).

### Example 55

### Synthesis of N-(2′-(4,4-difluorocyclohexyl)-[2,4′-bipyridin]-3′-yl)-2-isopropylpyrimidine-5-carboxamide

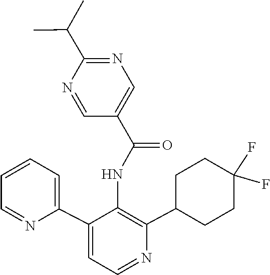 [SMILES: CC(C)c1ncc(C(=O)Nc2c(-c3ccccn3)ccnc2C2CCC(F)(F)CC2)cn1]

### Step 1. Preparation of N-(2′-chloro-[2,4′-bipyridin]-3′-yl)-2-isopropylpyrimidine-5-carboxamide

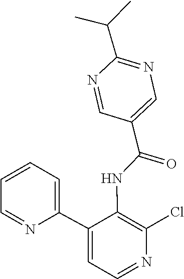 [SMILES: CC(C)c1ncc(C(=O)Nc2c(-c3ccccn3)ccnc2Cl)cn1]

To a solution of N-(2-chloro-4-iodo-3-pyridyl)-2-isopropyl-pyrimidine-5-carboxamide (0.15 g, 0.36 mmol) in THF (2.0 mL) were added tetrakis(triphenylphosphine)palladium(0) (0.042 g, 0.036 mmol) and 2-pyridylzinc bromide (1.4 mL, 0.72 mmol). After stirring the mixture under nitrogen at 70° C. for 16 h, the second tetrakis(triphenylphosphine)palladium(0) (0.042 g, 0.036 mmol) and 2-pyridylzinc bromide (1.4 mL, 0.72 mmol) were added and stirred at 70° C. for additional 8 h. After cooling to ambient temperature, the mixture was diluted with ethyl acetate (15 mL) was washed with 1M HCl solution (2×20 mL), brine (20 mL), and concentrated in vacuo. The residue was purified by column chromatography, eluting with a gradient of 10 to 100% ethyl acetate in heptane, to afford the title compound as a pale yellow solid (0.10 g, 80% yield): MS (ES+) m/z 354.0 (M+1), 356.0 (M+1).

### Step 2. Preparation of N-(2′-(4,4-difluorocyclohexyl)-[2,4′-bipyridin]-3′-yl)-2-isopropylpyrimidine-5-carboxamide

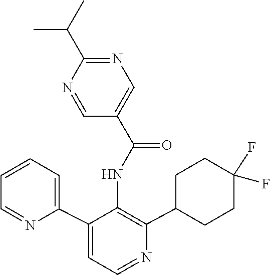 [SMILES: CC(C)c1ncc(C(=O)Nc2c(-c3ccccn3)ccnc2C2CCC(F)(F)CC2)cn1]

To a mixture of (4,4″-di-t-butyl-2,2″-bipyridine)bis[3,5-difluoro-2-[5-trifluoromethyl-2-pyridinyl-KN)phenyl-KC]iridium(III) hexafluorophosphate (0.0024 g, 0.0021 mmol), NiCl₂.glyme (0.0047 g, 0.021 mmol), 4,4′-di-tert-butyl-2,2′-bipyridine (0.0086 g, 0.032 mmol), N-(2-chloro-4-(3,4-dihydro-2H-pyran-6-yl)pyridin-3-yl)-2-isopropylpyrimidine-5-carboxamide (0.073 g, 0.21 mmol), cesium carbonate (0.14 g, 0.43 mmol) and 4,4-difluorocyclohexanecarboxylic acid (0.070 g, 0.43 mmol) was added N,N-dimethylformamide (5.4 mL). The mixture was purged with nitrogen for 10 s and sealed. The reaction mixture was then stirred in front of Kessil PR160L lights (440 nm) for 20 h. The reaction mixture was quenched with saturated aqueous sodium bicarbonate solution (10 mL) and extracted with ethyl acetate (3×10 mL). The combined organic extracts were washed with water (3×25 mL) and brine (50 mL), dried over anhydrous sodium sulfate, and concentrated in vacuo. Purification of the residue by preparative reverse phase HPLC, using 10-85% acetonitrile in water containing 0.5% of formic acid as eluent, afforded the title compound as a colorless solid (0.031 g, 33% yield): ¹H-NMR (400 MHz; DMSO-d₆): δ 10.46 (s, 1H), 9.06 (s, 2H), 8.65 (d, J=5.0 Hz, 2H), 7.87 (td, J=7.7, 1.8 Hz, 1H), 7.65 (s, 1H), 7.52 (d, J=4.9 Hz, 1H), 7.41-7.39 (m, 1H), 3.21 (dquintet, J=13.8, 6.9 Hz, 2H), 2.12-1.99 (m, 3H), 1.92-1.85 (m, 5H), 1.30 (d, J=6.9 Hz, 6H); MS (ES+) m/z 438.2 (M+1).

### Example 56

### Synthesis of N-(2-(4,4-difluorocyclohexyl)-4-(2,5-difluorophenyl)pyridin-3-yl)-6-(2-(dimethylamino)ethoxy)-5-fluoronicotinamide

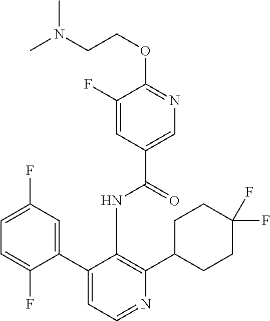 [SMILES: CN(C)CCOc1ncc(C(=O)Nc2c(-c3cc(F)ccc3F)ccnc2C2CCC(F)(F)CC2)cc1F]

### Step 1. Preparation of N-(2-(4,4-difluorocyclohexyl)-4-(2,5-difluorophenyl)pyridin-3-yl)-5,6-difluoronicotinamide

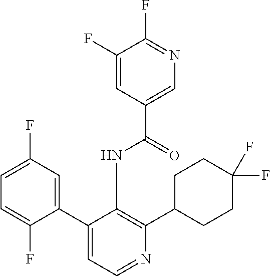 [SMILES: O=C(Nc1c(-c2cc(F)ccc2F)ccnc1C1CCC(F)(F)CC1)c1cnc(F)c(F)c1]

To a mixture of 2-(4,4-difluorocyclohexyl)-4-(2,5-difluorophenyl)pyridin-3-amine (1.00 g, 3.08 mmol), 5,6-difluoropyridine-3-carboxylic acid (0.539 g, 3.39 mmol), 2-chloro-1-methyl-pyridin-1-ium iodide (1.02 g, 4.01 mmol)) in tetrahydrofuran (10 mL) was added N,N-diisopropylethylamine (1.20 g, 9.25 mmol). The mixture was stirred at 70° C. for 12 h. The reaction mixture was cooled to ambient temperature and concentrated under reduced pressure. The residue was purified by flash silica gel chromatography, eluting with a gradient of 0-25% of ethyl acetate in petroleum ether, to afford the title compound as a colorless solid (1.00 g, 2.15 mmol, 78% yield): ¹H NMR (400 MHz, DMSO-d₆) δ 10.38 (s, 1H), 8.64 (d, J=4.8 Hz, 1H), 8.39 (t, J=1.8 Hz, 1H), 8.28 (dt, J=2.0, 9.6 Hz, 1H), 7.39 (d, J=5.4 Hz, 1H), 7.37-7.20 (m, 3H), 3.24-3.11 (m, 1H), 2.17-2.04 (m, 2H), 1.98-1.74 (m, 6H).

### Step 2. Preparation of N-(2-(4,4-difluorocyclohexyl)-4-(2,5-difluorophenyl)pyridin-3-yl)-6-(2-(dimethylamino)ethoxy)-5-fluoronicotinamide

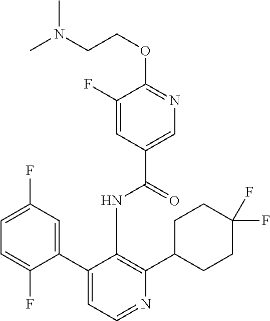 [SMILES: CN(C)CCOc1ncc(C(=O)Nc2c(-c3cc(F)ccc3F)ccnc2C2CCC(F)(F)CC2)cc1F]

To a solution of N-(2-(4,4-difluorocyclohexyl)-4-(2,5-difluorophenyl)pyridin-3-yl)-5,6-difluoronicotinamide (0.0500 g, 0.107 mmol) and 2-(dimethylamino)ethanol (0.0192 g, 0.215 mmol) in tetrahydrofuran (1 mL) was added potassium tert-butoxide (1 M in tetrahydrofuran, 0.200 mL) at 0° C., and the mixture was stirred at 25° C. for 12 h. To the mixture was added water (10 mL), then the mixture was extracted with ethyl acetate (3×10 mL). The combined organic layers were washed with brine (30 mL), dried over sodium sulfate, filtered, and concentrated under reduced pressure. The residue was purified by preparative HPLC, eluting with a gradient of 15-35% of acetonitrile in water containing 0.225% formic acid, to afford the title compound as a colorless solid (0.0335 g, 52% yield): ¹H NMR (400 MHz, MeOD-d₄) δ 8.60 (d, J=4.8 Hz, 1H), 8.53 (s, 1H), 8.36 (d, J=2.0 Hz, 1H), 7.81 (dd, J=2.0, 10.6 Hz, 1H), 7.35 (d, J=4.8 Hz, 1H), 7.27-7.06 (m, 3H), 4.64-4.61 (m, 2H), 3.19-3.11 (m, 1H), 2.91 (t, J=5.6 Hz, 2H), 2.42 (s, 6H), 2.19-2.00 (m, 4H), 1.94-1.77 (m, 4H); MS (ES+) m/z 535.3 (M+1).

### Example 57-60

In a similar manner as described in Example 56, utilizing the appropriately substituted starting materials and intermediates, the following compounds were prepared:

| Example | Structure | | MS (ES+) | |
|---|---|---|---|---|
| No. | Name | Yield | m/z | ¹H-NMR |
| 57 | 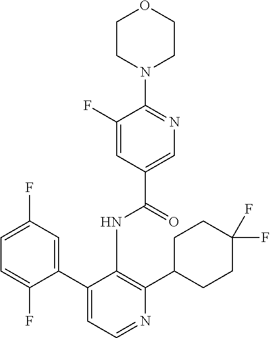 [SMILES: O=C(Nc1c(-c2cc(F)ccc2F)ccnc1C1CCC(F)(F)CC1)c1cnc(N2CCOCC2)c(F)c1] | 45% | 533.2 (M + 1) | (400 MHz, CDCl₃) δ 8.67 (d, J = 4.8 Hz, 1H), 8.30 (s, 1H), 7.70-7.54 (m, 1H), 7.40 (s, 1H), 7.20 (d, J = 4.8 Hz, 1H), 7.16-7.03 (m, 3H), 3.87-3.77 (m, 4H), 3.73-3.65 (m, 4H), 3.08-2.91 (m, 1H), 2.29-2.19 (m, 2H), 2.17-2.05 (m, 2H), 1.95 (d, J = 13.2 Hz, 2H), 1.86-1.72 (m, 2H) |
| | N-(2-(4,4- | | | |
| | difluorocyclohexyl)-4-(2,5- | | | |
| | difluorophenyl)pyridin-3-yl)- | | | |
| | 5-fluoro-6- | | | |
| | morpholinonicotinamide | | | |
| 58 | 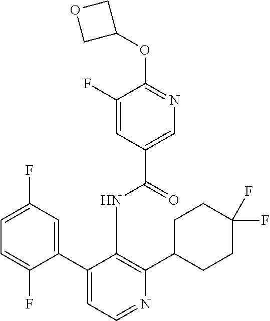 [SMILES: O=C(Nc1c(-c2cc(F)ccc2F)ccnc1C1CCC(F)(F)CC1)c1cnc(OC2COC2)c(F)c1] | 59% | 519.2 (M + 1) | (400 MHz; CDCl₃) δ 8.70 (d, J = 4.9 Hz, 1H), 8.21 (d, J = 1.9 Hz, 1H), 7.76 (dd, J = 9.9, 1.9 Hz, 1H), 7.51 (s, 1H), 7.22 (d, J = 4.9 Hz, 1H), 7.18-7.06 (m, 3H), 5.72 (quintet, J = 5.9 Hz, 1H), 5.03 (t, J = 7.2 Hz, 2H), 4.82 (dd, J = 7.9, 5.5 Hz, 2H), 3.02- 2.95 (m, 1H), 2.29- 2.21 (m, 2H), 2.18- 2.10 (m, 2H), 1.97- 1.93 (m, 2H), 1.86- 1.70 (m, 2H) |
| | N-(2-(4,4- | | | |
| | difluorocyclohexyl)-4-(2,5- | | | |
| | difluorophenyl)pyridin-3-yl)- | | | |
| | 5-fluoro-6-(oxetan-3- | | | |
| | yloxy)nicotinamide | | | |
| 59 | 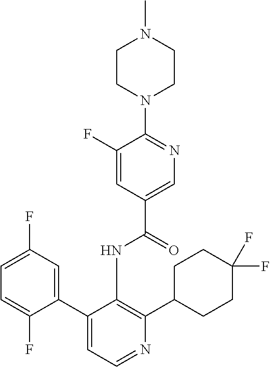 [SMILES: CN1CCN(c2ncc(C(=O)Nc3c(-c4cc(F)ccc4F)ccnc3C3CCC(F)(F)CC3)cc2F)CC1] | 58% | 546.3 (M + 1) | (400 MHz, DMSO- d₆) δ 9.97 (s, 1H), 8.60 (d, J = 4.8 Hz, 1H), 8.40-8.37 (m, 1H), 7.74 (dd, J = 14.8, 2.0 Hz, 1H), 7.36-7.29 (m, 2H), 7.28-7.23 (m, 1H), 7.23-7.17 (m, 1H), 3.59-3.53 (m, 4H), 3.18-3.11 (m, 1H), 2.45-2.37 (m, 4H), 2.20 (s, 3H), 2.15-2.03 (m, 2H), 1.91 (s, 2H), 1.83 (s, 4H) |
| | N-(2-(4,4- | | | |
| | difluorocyclohexyl)-4-(2,5- | | | |
| | difluorophenyl)pyridin-3-yl)- | | | |
| | 5-fluoro-6-(4- | | | |
| | methylpiperazin-1- | | | |
| | yl)nicotinamide | | | |
| 60 | 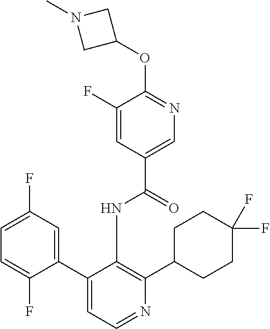 | 35% | 523.2 (M + 1) | (400 MHz; MeOD) δ 8.62 (d, J = 5.0 Hz, 1H), 8.34 (d, J = 2.0 Hz, 1H), 7.86 (dd, J = 10.5, 2.0 Hz, 1H), 7.39- 7.36 (m, 1H), 7.24- 7.12 (m, 3H), 5.42- 5.39 (m, 1H), 4.03- 3.99 (m, 2H), 3.52- 3.49 (m, 2H), 3.19- 3.11 (m, 1H), 2.55 (s, 3H), 2.20-2.01 (m, 4H), 1.94-1.87 (m, 2H), 1.87-1.77 (m, 2H) |
| | N-(2-(4,4- | | | |
| | difluorocyclohexyl)-4-(2,5- | | | |
| | difluorophenyl)pyridin-3-yl)- | | | |
| | 5-fluoro-6-((1- | | | |
| | methylazetidin-3- | | | |
| | yl)oxy)nicotinamide | | | |
| 61 | 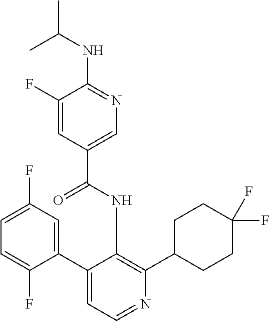 [SMILES: CC(C)Nc1ncc(C(=O)Nc2c(-c3cc(F)ccc3F)ccnc2C2CCC(F)(F)CC2)cc1F] | 38% | 505.2 (M + 1) | ¹H NMR (400 MHz, MeOD-d₄) δ 8.57 (d, J = 4.8 Hz, 1H), 8.28 (d, J = 1.2 Hz, 1H), 7.51 (dd, J = 12.0, 2.0 Hz, 1H), 7.33 (d, J = 4.8 Hz, 1H), 7.25-7.04 (m, 3H), 4.30 (dt, J = 13.2, 6.4 Hz, 1H), 3.22-3.09 (m, 1H), 2.21-2.10 (m, 2H), 2.09-1.97 (m, 2H), 1.90 (d, J = 13.6 Hz, 3H), 1.84-1.72 (m, 1H), 1.25 (d, J = 6.8 Hz, 6H) |
| | N-(2-(4,4- | | | |
| | difluorocyclohexyl)-4-(2,5- | | | |
| | difluoro-phenyl)pyridin-3- | | | |
| | yl)-5-fluoro-6- | | | |
| | (isopropylamino)nicotinamide | | | |
| 62 | 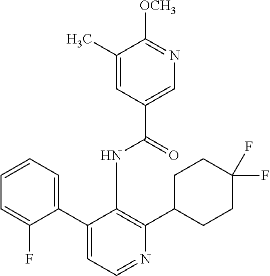 [SMILES: COc1ncc(C(=O)Nc2c(-c3ccccc3F)ccnc2C2CCC(F)(F)CC2)cc1C] | 79% | 456.1 (M + 1) | ¹H NMR (400 MHz, MeOD-d₄) δ 8.57 (d, J = 5.0 Hz, 1H), 8.34 (d, J = 2.0 Hz, 1H), 7.77 (dd, J = 2.2, 0.8 Hz, 1H), 7.42- 7.31 (m, 3H), 7.23-7.13 (m, 2H), 3.99 (s, 3H), 3.21- 3.08 (m, 1H), 2.19 (s, 3H), 2.17-2.00 (m, 4H), 1.98-1.72 (m, 4H) |
| | N-(2-(4,4- | | | |
| | difluorocyclohexyl)-4-(2- | | | |
| | fluorophenyl) pyridin-3-yl)-6- | | | |
| | methoxy-5- | | | |
| | methylnicotinamide | | | |

### Example 63

### Synthesis of (S)-2-(4,4-difluorocyclohexyl)-4-(2,5-difluorophenyl)-N-(2-(2-methylazetidin-1-yl)pyrimidin-5-yl)nicotinamide

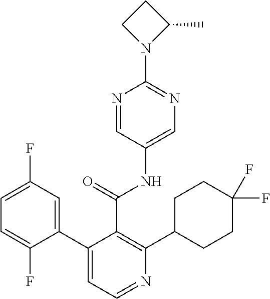 [SMILES: C[C@H]1CCN1c1ncc(NC(=O)c2c(-c3cc(F)ccc3F)ccnc2C2CCC(F)(F)CC2)cn1]

### Step 1. Preparation of tert-butyl 2-chloro-4-(2,5-difluorophenyl) nicotinate

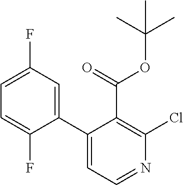 [SMILES: CC(C)(C)OC(=O)c1c(-c2cc(F)ccc2F)ccnc1Cl]

To a solution of 2-chloro-4-(2,5-difluorophenyl)nicotinic acid (1.00 g, 3.71 mmol) in tert-butanol (10 mL) was added di-tert-butyl dicarbonate (1.62 g, 7.42 mmol), triethylamine (1.13 g, 11.1 mmol) and 4-dimethylaminopyridine (0.0453 g, 0.371 mmol). The mixture was stirred at 50° C. for 12 h. The reaction mixture was cooled to ambient temperature and concentrated under reduced pressure. The residue was purified by flash silica gel chromatography, eluting with a gradient of 0-4% of ethyl acetate in petroleum ether, to afford the title compound as a colorless solid (0.800 g, 2.46 mmol, 66% yield): ¹H NMR (400 MHz, CDCl₃) δ 8.49 (d, J=5.0 Hz, 1H), 7.32-7.25 (m, 1H), 7.22-7.05 (m, 3H), 1.41 (s, 9H).

### Step 2. Preparation of tert-butyl 2-(4,4-difluorocyclohex-1-en-1-yl)-4-(2,5-difluorophenyl)nicotinate

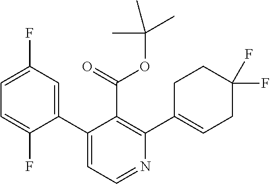 [SMILES: CC(C)(C)OC(=O)c1c(-c2cc(F)ccc2F)ccnc1C1=CCC(F)(F)CC1]

To a mixture of tert-butyl 2-chloro-4-(2,5-difluorophenyl)nicotinate (0.700 g, 2.15 mmol), 2-(4,4-difluorocyclohexen-1-yl)-4,4,5,5-tetramethyl-1,3,2-dioxaborolane (0.305 g, 1.25 mmol), [1,1-bis(diphenylphosphino)ferrocene]dichloropalladium(II) (0.157 g, 0.215 mmol) and potassium carbonate (0.891 g, 6.45 mmol) was added dioxane (10 mL)/water (2 mL). The mixture was degassed and purged with nitrogen 3 times, and then the mixture was stirred at 100° C. for 12 h under a nitrogen atmosphere. The reaction mixture was cooled to ambient temperature. The reaction mixture was concentrated under reduced pressure. The residue was purified by flash silica gel chromatography, eluting with a gradient of 0-10% of ethyl acetate in petroleum ether, to afford the title compound as a colorless solid (0.540 g, 62% yield): ¹H NMR (400 MHz, CDCl₃) δ 8.66 (d, J=4.8 Hz, 1H), 7.20 (d, J=4.8 Hz, 1H), 7.16-7.09 (m, 2H), 7.08-7.01 (m, 1H), 5.74 (s, 1H), 2.88 (t, J=5.6 Hz, 2H), 2.68 (t, J=13.2 Hz, 2H), 2.24 (tt, J=13.6, 6.8 Hz, 2H), 1.35 (s, 9H).

### Step 3. Preparation of tert-butyl 2-(4,4-difluorocyclohexyl)-4-(2,5-difluorophenyl)nicotinate

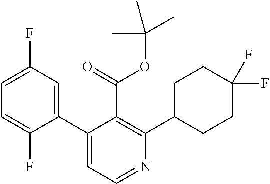 [SMILES: CC(C)(C)OC(=O)c1c(-c2cc(F)ccc2F)ccnc1C1CCC(F)(F)CC1]

To a solution of tert-butyl 2-(4,4-difluorocyclohex-1-en-1-yl)-4-(2,5-difluorophenyl)nicotinate (0.540 g, 1.33 mmol) in methanol (10 mL) was added palladium on carbon(0.450 g, 0.423 mmol, 10 wt %) under a nitrogen atmosphere. The suspension was degassed under vacuum and purged with hydrogen several times. The mixture was stirred under hydrogen (15 psi) at 25° C. for 12 h. The reaction mixture was filtered and concentrated under reduced pressure to give the title compound as a colorless solid (0.450 g, crude): ¹H NMR (400 MHz, CDCl₃) δ 8.64 (d, J=5.0 Hz, 1H), 7.18-7.07 (m, 3H), 7.03 (ddd, J=8.0, 5.6, 2.8 Hz, 1H), 3.03 (t, J=11.6 Hz, 1H), 2.31-2.22 (m, 2H), 2.19-2.08 (m, 2H), 2.03-1.95 (m, 2H), 1.90-1.74 (m, 2H), 1.34 (s, 9H).

### Step 4. Preparation of 2-(4,4-difluorocyclohexyl)-4-(2,5-difluorophenyl)nicotinic acid

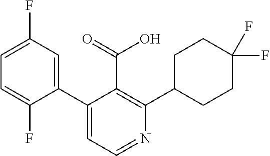 [SMILES: O=C(O)c1c(-c2cc(F)ccc2F)ccnc1C1CCC(F)(F)CC1]

To a solution of tert-butyl 2-(4,4-difluorocyclohexyl)-4-(2,5-difluorophenyl)nicotinate (0.450 g, 1.10 mmol) in dichloromethane (10 mL) was added trifluoroacetic acid (7.70 g, 67.5 mmol). The mixture was stirred at 25° C. for 12 h. The reaction mixture was concentrated under reduced pressure. The residue was purified by reversed-phase column chromatography, eluting with 0.1% formic acid in water. To the product solution was added 1 mL of concentrated hydrochloric acid. The resulting solution was lyophilized to give the title compound as a colorless solid (0.310 g, 72% yield): ¹H NMR (400 MHz, DMSO-d₆) δ 8.69 (d, J=4.8 Hz, 1H), 8.14 (s, 1H), 7.44-7.33 (m, 3H), 7.26 (ddd, J=8.4, 5.2, 2.8 Hz, 1H), 3.16-3.00 (m, 1H), 2.13 (d, J=6.8 Hz, 2H), 2.04-1.84 (m, 6H).

### Step 5. Preparation of (S)-2-(2-methylazetidin-1-yl)-5-nitropyrimidine

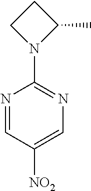

To a solution of (S)-2-methylazetidine hydrochloride (0.148 g, 1.38 mmol) and cesium carbonate (1.23 g, 3.76 mmol) in dimethylformamide (2 mL) was added 2-chloro-5-nitropyrimidine (0.200 g, 1.25 mmol) at 25° C. Then the mixture was stirred at 25° C. for 12 h. The residue was diluted with ethyl acetate (20 mL) and washed with water (3×20 mL), dried over sodium sulfate, filtered, and concentrated under reduced pressure to give the title compound as a colorless solid (0.210 g, 82% yield): ¹H NMR (400 MHz, DMSO-d₆) δ 9.07 (s, 2H), 4.69-4.57 (m, 1H), 4.23-4.05 (m, 2H), 2.60-2.53 (m, 1H), 2.04-1.94 (m, 1H), 1.50 (d, J=6.4 Hz, 3H).

### Step 6. Preparation of (S)-2-(2-methylazetidin-1-yl)pyrimidin-5-amine

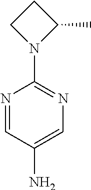

To a solution of (S)-2-(2-methylazetidin-1-yl)-5-nitropyrimidine (0.210 g, 1.08 mmol) in ethyl acetate (10 mL) was added palladium on carbon (0.0300 g, 10 wt %) at 25° C. under a nitrogen atmosphere. The suspension was degassed and purged with hydrogen for three times. The mixture was stirred at 25° C. for 1 h under hydrogen (15 psi, balloon). The resulting mixture was filtered over Celite. The filter cake was washed with ethyl acetate (10 mL). The filtrate was concentrated under reduced pressure to give the title compound as a yellow solid (0.120 g, 64% yield): ¹H NMR (400 MHz, DMSO-d₆) δ 7.85 (s, 2H), 4.57 (s, 2H), 4.22-4.10 (m, 1H), 3.76 (dt, J=8.4, 4.0 Hz, 1H), 3.67 (q, J=8.0 Hz, 1H), 2.26 (dtd, J=10.4, 8.4, 4.0 Hz, 1H), 1.96-1.84 (m, 1H), 1.37 (d, J=6.0 Hz, 3H).

### Step 7. Preparation of (S)-2-(4,4-difluorocyclohexyl)-4-(2,5-difluorophenyl)-N-(2-(2-methylazetidin-1-yl)pyrimidin-5-yl)nicotinamide

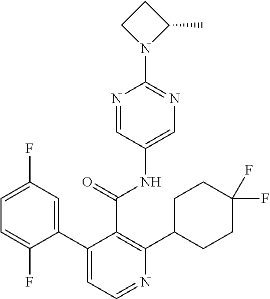 [SMILES: C[C@H]1CCN1c1ncc(NC(=O)c2c(-c3cc(F)ccc3F)ccnc2C2CCC(F)(F)CC2)cn1]

To a solution of (S)-2-(2-methylazetidin-1-yl)pyrimidin-5-amine (0.0400 g, 0.244 mmol), 2-(4,4-difluorocyclohexyl)-4-(2,5-difluorophenyl)nicotinic acid (0.0880 g, 0.249 mmol) and N,N-diisopropylethylamine (0.0960 g, 0.743 mmol) in tetrahydrofuran (2 mL) was added 2-chloro-1-methylpyridinium iodide (0.0750 g, 0.294 mmol) at 25° C. The mixture was stirred at 70° C. for 12 h. After cooling to ambient temperature, the mixture was concentrated under reduced pressure. The residue was purified by preparative HPLC, eluting with a gradient of 46-76% of acetonitrile in water containing 0.225% formic acid, to afford the title compound as a colorless solid (0.0420 g, 34% yield) as a white solid: ¹H NMR (400 MHz, MeOD-d₄) δ 8.67 (d, J=4.8 Hz, 1H), 8.23 (s, 2H), 7.37 (dd, J=4.8, 1.2 Hz, 1H), 7.30-7.14 (m, 3H), 4.52-4.40 (m, 1H), 4.10-3.99 (m, 1H), 3.98-3.85 (m, 1H), 3.12-3.00 (m, 1H), 2.47 (dtd, J=10.8, 8.8, 5.2 Hz, 1H), 2.22-2.07 (m, 4H), 2.06-1.91 (m, 4H), 1.90-1.79 (m, 1H), 1.48 (d, J=6.0 Hz, 3H); MS (ES+) m/z 500.2 (M+1).

### Example 64-74

In a similar manner as described in Example 63, utilizing the appropriately substituted starting materials and intermediates, the following compounds were prepared:

| Example | Structure | | MS (ES+) | |
|---|---|---|---|---|
| No. | Name | Yield | m/z | ¹H-NMR |
| 64 | 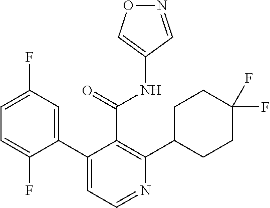 [SMILES: O=C(Nc1cnoc1)c1c(-c2cc(F)ccc2F)ccnc1C1CCC(F)(F)CC1] | 27% | 420.1 (M + 1) | (400 MHz, DMSO- d₆) δ 10.83 (s, 1H), 9.13 (s, 1H), 8.73 (d, J = 4.8 Hz, 1H), 8.51 (s, 1H), 7.40 (dd, J = 4.8, 1.2 Hz, 1H), 7.36 (td, J = 9.2, 4.4 Hz, 1H), 7.33-7.27 (m, 1H), 7.23 (ddd, J = 8.8, 5.6, 3.2 Hz, 1H), 2.97-2.90 (m, 1H), 2.13-2.06 (m, 2H), 2.01-1.84 |
| | 2-(4,4- | | | (m, 6H) |
| | difluorocyclo- | | | |
| | hexyl)-4- (2,5- | | | |
| | difluorophenyl)- | | | |
| | N-(isoxazol-4- | | | |
| | yl)nicotinamide | | | |
| 65 | 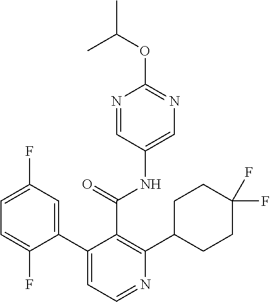 [SMILES: CC(C)Oc1ncc(NC(=O)c2c(-c3cc(F)ccc3F)ccnc2C2CCC(F)(F)CC2)cn1] | 5% | 489.0 (M + 1) | (400 MHz, DMSO- d₆) δ 10.8 (s, 1H), 8.73 (d, J = 5.2 Hz, 1H), 8.55 (s, 1H), 7.43-7.30 (m, 5H), 5.16-5.09 (m, 1H), 3.05 (br s, 1H), 2.08-1.93 (m, 8H), 1.29 (d, J = 6.0 Hz, 6H) |
| | 2-(4,4- | | | |
| | difluorocyclo- | | | |
| | hexyl)-4-(2,5- | | | |
| | difluorophenyl)- | | | |
| | N-(2- | | | |
| | isopropoxy- | | | |
| | pyrimidin-5- | | | |
| | yl)nicotinamide | | | |
| 66 | 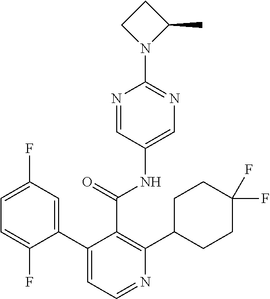 [SMILES: C[C@@H]1CCN1c1ncc(NC(=O)c2c(-c3cc(F)ccc3F)ccnc2C2CCC(F)(F)CC2)cn1] | 22% | 500.2 (M + 1) | (400 MHz, MeOD- d₄) δ 8.67 (d, J = 4.8 Hz, 1H), 8.23 (s, 2H), 7.37 (dd, J = 4.8, 1.2 Hz, 1H), 7.33-7.15 (m, 3H), 4.51-4.40 (m, 1H), 4.03 (dt, J = 8.8, 5.2 Hz, 1H), 3.98- 3.89 (m, 1H), 3.11-3.00 (m, 1H), 2.48 (dtd, J = 10.8, 8.8, 4.8 Hz, 1H), 2.22-2.08 (m, 4H), 2.06-1.92 (m, 4H), 1.90-1.80 (m, 1H), 1.48 (d, J = 6.2 Hz, 3H), 1.29 (s, 2H) |
| | (R)-2-(4,4- | | | |
| | difluorocyclo- | | | |
| | hexyl)-4-(2,5- | | | |
| | difluorophenyl)- | | | |
| | N-(2-(2- | | | |
| | methylazetidin- | | | |
| | 1-yl)pyrimidin-5- | | | |
| | yl)nicotinamide | | | |
| 67 | 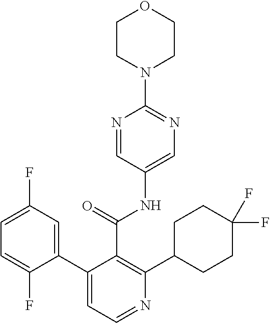 [SMILES: O=C(Nc1cnc(N2CCOCC2)nc1)c1c(-c2cc(F)ccc2F)ccnc1C1CCC(F)(F)CC1] | 8% | 516.1 (M + 1) | (400 MHz, MeOD- d₄) δ 8.84-8.82 (d, J = 5.6 Hz, 1H), 8.51 (s, 2H), 7.86-7.82 (dd, J = 0.8 Hz, 5.6 Hz, 1H), 7.37-7.26 (m, 3H), 3.77 (s, 8H), 3.27-3.21 (m, 1H), 2.26-2.12 (m, 6H), 2.06-1.88 (m, 2H) |
| | 2-(4,4- | | | |
| | difluorocyclo- | | | |
| | hexyl)-4-(2,5- | | | |
| | difluorophenyl)- | | | |
| | N-(2- | | | |
| | morpholino- | | | |
| | pyrimidin-5- | | | |
| | yl)nicotinamide | | | |
| 68 | 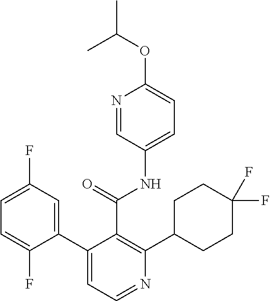 [SMILES: CC(C)Oc1ccc(NC(=O)c2c(-c3cc(F)ccc3F)ccnc2C2CCC(F)(F)CC2)cn1] | 23% | 488.2 (M + 1) | (400 MHz, CDCl₃) δ 8.71 (d, J = 4.8 Hz, 1H), 7.87 (d, J = 2.4 Hz, 1H), 7.62 (dd, J = 8.8, 2.8 Hz, 1H), 7.20 (d, J = 4.8 Hz, 1H), 7.17-7.06 (m, 4H), 6.66 (d, J = 8.8 Hz, 1H), 5.30-5.14 (m, 1H), 3.18-3.00 (m, 1H), 2.27-2.14 (m, 4H), 2.04-1.83 (m, 4H), 1.33 (d, J = 6.0 Hz, 6H) |
| | 2-(4,4- | | | |
| | difluorocyclo- | | | |
| | hexyl)-4-(2,5- | | | |
| | difluorophenyl)- | | | |
| | N-(6- | | | |
| | isopropoxy- | | | |
| | pyridin-3- | | | |
| | yl)nicotinamide | | | |
| 69 | 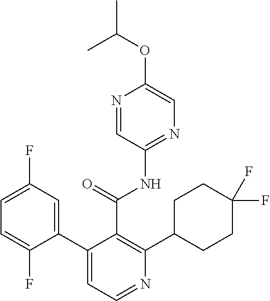 [SMILES: CC(C)Oc1cnc(NC(=O)c2c(-c3cc(F)ccc3F)ccnc2C2CCC(F)(F)CC2)cn1] | 12% | 489.1 (M + 1) | (400 MHz, DMSO- d₆) δ 11.00 (s, 1H), 8.70 (d, J = 5.2 Hz, 1H), 8.66 (s, 1H), 7.99 (d, J = 0.8 Hz, 1H), 7.40- 7.22 (m, 4H), 5.25-5.07 (m, 1H), 3.01 (t, J = 10.0 Hz, 1H), 2.08 (dd, J = 11.2, 6.4 Hz, 2H), 2.03-1.78 (m, 6H), 1.30 (d, J = 6.0 Hz, 6H) |
| | 2-(4,4- | | | |
| | difluorocyclo- | | | |
| | hexyl)-4-(2,5- | | | |
| | difluorophenyl)- | | | |
| | N-(5- | | | |
| | isopropoxy- | | | |
| | pyrazin-2- | | | |
| | yl)nicotinamide | | | |
| 70 | 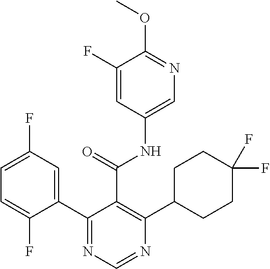 [SMILES: COc1ncc(NC(=O)c2c(-c3cc(F)ccc3F)ncnc2C2CCC(F)(F)CC2)cc1F] | 28% | 479.2 (M + 1) | ¹H NMR (400 MHz, DMSO-d₆) δ 10.79 (s, 1H), 9.35 (s, 1H), 7.92 (d, J = 2.0 Hz, 1H), 7.81 (dd, J = 11.6, 2.0 Hz, 1H), 7.44- 7.34 (m, 3H), 3.91 (s, 3H), 3.16-3.07 (m, 1H), 2.11 (s, 2H), 1.96 (d, J = 5.2 Hz, 6H) |
| | 4-(4,4- | | | |
| | difluorocyclo- | | | |
| | hexyl)-6-(2,5- | | | |
| | difluorophenyl)- | | | |
| | N-(5-fluoro-6- | | | |
| | methoxypyridin- | | | |
| | 3-yl)pyrimidine- | | | |
| | 5-carboxamide | | | |
| 71 | 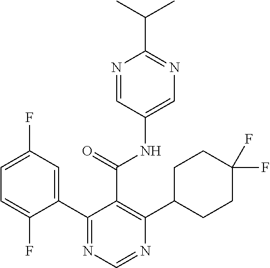 [SMILES: CC(C)c1ncc(NC(=O)c2c(-c3cc(F)ccc3F)ncnc2C2CCC(F)(F)CC2)cn1] | 7% | 473.2 (M + 1) | ¹H-NMR (400 MHz; DMSO-d₆) δ 10.97 (s, 1H), 9.37 (s, 1H), 8.77 (s, 2H), 7.43-7.38 (m, 3H), 3.16-3.08 (m, 2H), 2.14-2.04 (m, 2H), 2.03-1.92 (m, 6H), 1.25 (d, J = 6.9 Hz, 7H) |
| | 4-(4,4- | | | |
| | difluorocyclo- | | | |
| | hexyl)-6-(2,5- | | | |
| | difluorophenyl)- | | | |
| | N-(2- | | | |
| | isopropyl- | | | |
| | pyrimidin-5- | | | |
| | yl)pyrimidine-5- | | | |
| | carboxamide | | | |
| 72 | 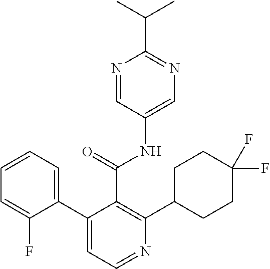 [SMILES: CC(C)c1ncc(NC(=O)c2c(-c3ccccc3F)ccnc2C2CCC(F)(F)CC2)cn1] | 8% | 455.1 (M + 1) | ¹H NMR (400 MHz, MeOD-d₄) δ 8.71 (s, 2H), 8.67 (d, J = 5.0 Hz, 1H), 8.52 (s, 1H), 7.48-7.39 (m, 2H), 7.38 (dd, J = 5.0, 1.4 Hz, 1H), 7.26- 7.17 (m, 2H), 3.14 (quin, J = 7.0 Hz, 1H), 3.06 (t, J = 11.8 Hz, 1H), 2.23-2.08 (m, 4H), 2.04-1.80 (m, 4H), 1.29 (d, J = 7.0 Hz, 6H) |
| | 2-(4,4- | | | |
| | difluorocyclo- | | | |
| | hexyl)-4-(2- | | | |
| | fluorophenyl)- N- | | | |
| | (2- | | | |
| | isopropyl- | | | |
| | pyrimidin-5- | | | |
| | yl)nicotinamide | | | |
| 73 | 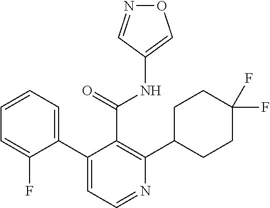 [SMILES: O=C(Nc1cnoc1)c1c(-c2ccccc2F)ccnc1C1CCC(F)(F)CC1] | 18% | 402.0 (M + 1) | ¹H NMR (400 MHz, CDCl₃) δ 8.90 (s, 1H), 8.73 (d, J = 5.0 Hz, 1H), 8.15 (s, 1H), 7.45-7.31 (m, 2H), 7.26-7.14 (m, 3H), 7.07 (s, 1H), 3.00 (t, J = 11.4 Hz, 1H), 2.33-2.12 (m, 4H), 2.05-1.77 (m, 4H) |
| | 2-(4,4- | | | |
| | difluorocyclo- | | | |
| | hexyl)-4- (2- | | | |
| | fluorophenyl)-N- | | | |
| | (isoxazol-4- | | | |
| | yl)nicotinamide | | | |
| 74 | 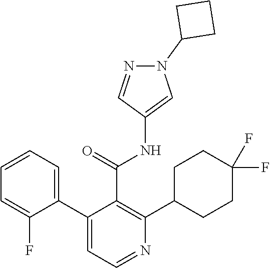 [SMILES: O=C(Nc1cnn(C2CCC2)c1)c1c(-c2ccccc2F)ccnc1C1CCC(F)(F)CC1] | 24% | 455.2 (M + 1) | ¹H NMR (400 MHz, MeOD-d₄) δ 8.63 (d, J = 5.0 Hz, 1H), 7.88 (s, 1H), 7.45-7.37 (m, 2H), 7.35 (s, 1H), 7.33 (dd, J = 5.0, 1.6 Hz, 1H), 7.22- 7.15 (m, 2H), 4.79-4.69 (m, 1H), 3.05-2.92 (m, 1H), 2.55-2.37 (m, 4H), 2.23-2.07 (m, 4H), 2.04-1.94 (m, 2H), 1.91-1.71 (m, 4H) |
| | N-(1-cyclobutyl- | | | |
| | 1H-pyrazol-4-yl)- | | | |
| | 2- (4,4- | | | |
| | difluorocyclo- | | | |
| | hexyl)-4-(2- | | | |
| | fluorophenyl) | | | |
| | nicotinamide | | | |

### Example 75

### Synthesis of 2-(4,4-difluorocyclohexyl)-4-(2-fluorophenyl)-N-(2-((tetrahydrofuran-3-yl)oxy)pyrimidin-5-yl)nicotinamide

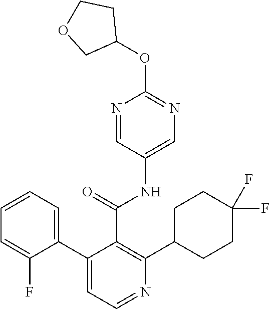 [SMILES: O=C(Nc1cnc(OC2CCOC2)nc1)c1c(-c2ccccc2F)ccnc1C1CCC(F)(F)CC1]

### Step 1. Preparation of 5-nitro-2-((tetrahydrofuran-3-yl)oxy)pyrimidine

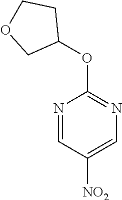 [SMILES: O=[N+]([O-])c1cnc(OC2CCOC2)nc1]

To a solution of 2-chloro-5-nitro-pyrimidine (0.100 g, 0.627 mmol) and tetrahydrofuran-3-ol (0.110 g, 1.25 mmol) in tetrahydrofuran (2 mL) was added potassium 2-methylpropan-2-olate (1 M in tetrahydrofuran, 0.94 mL) dropwise at 0° C. The mixture was stirred at 20° C. under a nitrogen atmosphere for 12 h. The mixture was poured into saturated aqueous sodium bicarbonate (10 mL) and extracted with ethyl acetate (3×20 mL). The combined organic layers were washed with brine (10 mL), dried over anhydrous sodium sulfate, filtered and the filtrate was evaporated under reduced pressure to afford the title compound as a light yellow oil (0.150 g, crude): ¹H NMR (400 MHz, CDCl₃) δ 9.31 (s, 2H), 5.71-5.62 (m, 1H), 4.14-4.09 (m, 1H), 4.07-4.02 (m, 1H), 3.99-3.96 (m, 2H), 2.32-2.20 (m, 2H).

### Step 2. Preparation of 2-((tetrahydrofuran-3-yl)oxy)pyrimidin-5-amine

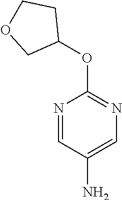 [SMILES: Nc1cnc(OC2CCOC2)nc1]

To a mixture of 5-nitro-2-tetrahydrofuran-3-yloxy-pyrimidine (0.150 g, crude) and ammonium chloride (0.760 g, 1.42 mmol) in ethanol (10 mL) and water (5 mL) was added iron powder (0.198 g, 3.55 mmol) in one portion at 20° C. The mixture was stirred at 90° C. for 12 h. The mixture was cooled to 20° C. and filtered. The filtrate was evaporated under reduced pressure. The residue was purified by flash silica gel chromatography, eluting with ethyl acetate, to afford the title compound as a colorless solid (0.0600 g, 46% yield): ¹H NMR (400 MHz, CDCl₃) δ 8.14 (s, 2H), 5.43 (s, 1H), 4.09 (m, 1H), 4.04-3.98 (m, 1H), 3.96-3.88 (m, 2H), 2.26-2.17 (m, 2H).

### Step 3. Preparation of 2-(4,4-difluorocyclohexyl)-4-(2-fluorophenyl)-N-(2-((tetrahydrofuran-3-yl)oxy)pyrimidin-5-yl)nicotinamide

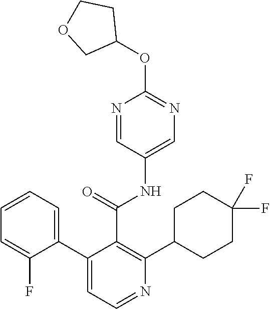 [SMILES: O=C(Nc1cnc(OC2CCOC2)nc1)c1c(-c2ccccc2F)ccnc1C1CCC(F)(F)CC1]

In a similar manner as described in Example 63, utilizing the appropriately substituted starting materials and intermediates, the title compound was afforded as a yellow solid (0.069 g, 24% yield): ¹H NMR (400 MHz, CDCl₃) δ 8.72 (d, J=5.2 Hz, 1H), 8.36 (s, 2H), 7.46-7.36 (m, 2H), 7.31 (br s, 1H), 7.27-7.16 (m, 3H), 5.50-5.42 (m, 1H), 4.12-4.05 (m, 1H), 4.03-3.96 (m, 1H), 3.95-3.88 (m, 2H), 3.15-3.00 (m, 1H), 2.31-2.14 (m, 6H), 2.02 (d, J=13.6 Hz, 2H), 1.94-1.81 (m, 2H); MS (ES+) m/z 499.1 (M+1).

### Example 76

### Synthesis of 2-(4,4-difluorocyclohexyl)-4-(2-fluorophenyl)-N-(2-isopropoxypyrimidin-5-yl)nicotinamide

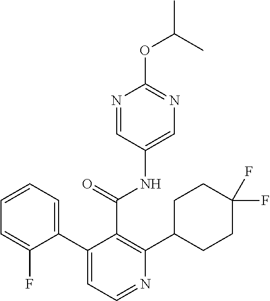 [SMILES: CC(C)Oc1ncc(NC(=O)c2c(-c3ccccc3F)ccnc2C2CCC(F)(F)CC2)cn1]

### Step 1. Preparation of 5-bromo-2-isopropoxypyrimidine

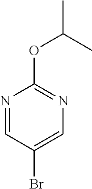

To a mixture of propan-2-ol (40 mL) was added sodium hydride (2.58 g, 64.6 mmol, 60 wt %) at 0° C. with stirring. The mixture was stirred at 60° C. for 30 min. To the mixture was added a solution of 5-bromo-2-chloropyrimidine (5.00 g, 25.9 mmol) in propan-2-ol (20 mL) at 0° C. and the resulting mixture was stirred at 90° C. for 12 h under a nitrogen atmosphere. The reaction mixture was quenched with water (50 mL) at 0° C. with stirring. The resulting mixture was extracted with ethyl acetate (3×50 mL). The combined organic layers were washed with brine (10 mL), dried over anhydrous sodium sulfate, filtered and the filtrate was concentrated in vacuo to give the title compound as a light-yellow oil (3.00 g, crude): ¹H NMR (400 MHz, DMSO-d₆) δ 8.73 (s, 2H), 5.25-5.06 (m, 1H), 1.31 (d, J=6.4 Hz, 6H).

### Step 2. Preparation of tert-butyl (2-isopropoxypyrimidin-5-yl)-carbamate

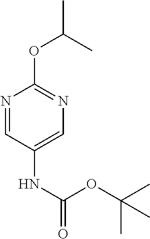 [SMILES: CC(C)Oc1ncc(NC(=O)OC(C)(C)C)cn1]

To a mixture of 5-bromo-2-isopropoxy-pyrimidine (1.00 g, 4.61 mmol), tert-butyl carbamate (0.648 g, 5.53 mmol) and cesium carbonate (4.50 g, 13.8 mmol) in dioxane (20 mL) was added [2-(2-aminophenyl)-phenyl]-methylsulfonyloxy-palladium di-tert-butyl-[2-(2,4,6-triisopropylphenyl)phenyl]phosphane (0.366 g, 0.461 mmol) in one portion at 20° C. The mixture was stirred at 70° C. under a nitrogen atmosphere for 12 h. The mixture was cooled to 20° C. and poured into water (10 mL). The mixture was extracted with ethyl acetate (3×30 mL). The combined organic layers were washed with brine (20 mL), dried over anhydrous sodium sulfate, filtered and the filtrate was evaporated under reduced pressure. The residue was purified by flash silica gel chromatography, eluting with a gradient of 30% of ethyl acetate in petroleum ether, to afford the title compound as a yellow solid (0.600 g, 51% yield): ¹H NMR (400 MHz, CDCl₃) δ 8.55 (s, 2H), 6.75-6.44 (m, 1H), 5.24-5.14 (m, 1H), 1.52 (s, 9H), 1.38 (d, J=6.4 Hz, 6H).

### Step 3. Preparation of 2-isopropoxypyrimidin-5-amine

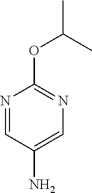

To a solution of tert-butyl N-(2-isopropoxypyrimidin-5-yl)carbamate (0.400 g, 1.58 mmol) in dichloromethane (10 mL) was added hydrogen chloride/dioxane (4 M, 10 mL) dropwise at 20° C. The mixture was stirred at 20° C. for 1 h. The mixture was evaporated under reduced pressure. The residue was purified by preparative HPLC, eluting with a gradient of water containing 0.1% ammonium hydroxide, to afford the title compound as a yellow solid (0.0550 g, 22% yield): ¹H NMR (400 MHz, DMSO-d₆) δ 7.93 (s, 2H), 5.03-4.95 (m, 1H), 4.91 (s, 2H), 1.24 (d, J=6.4 Hz, 6H).

### Step 4. Preparation of 2-(4,4-difluorocyclohexyl)-4-(2-fluorophenyl)-N-(2-isopropoxypyrimidin-5-yl)nicotinamide

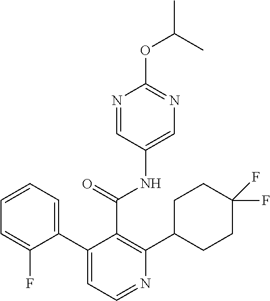 [SMILES: CC(C)Oc1ncc(NC(=O)c2c(-c3ccccc3F)ccnc2C2CCC(F)(F)CC2)cn1]

In a similar manner as described in Example 63, utilizing the appropriately substituted starting materials and intermediates, the title compound was afforded as a yellow solid (0.0059 g, 7% yield): ¹H NMR (400 MHz, CDCl₃) δ 8.71 (d, J=5.2 Hz, 1H), 8.31 (s, 2H), 7.45-7.35 (m, 2H), 7.26-7.17 (m, 3H), 7.15 (s, 1H), 5.26-5.15 (m, 1H), 3.15-3.01 (m, 1H), 2.29-2.14 (m, 4H), 2.05-1.96 (m, 2H), 1.92-1.81 (m, 2H), 1.37 (d, J=6.4 Hz, 6H); MS (ES+) m/z 471.1 (M+1).

### Example 77

### Synthesis of 2-(4,4-difluorocyclohexyl)-4-(2,5-difluorophenyl)-N-((1r,4r)-4-methoxycyclohexyl)nicotinamide

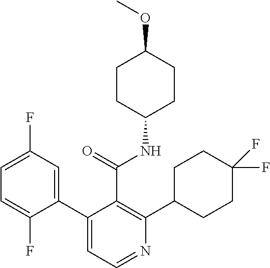 [SMILES: CO[C@H]1CC[C@H](NC(=O)c2c(-c3cc(F)ccc3F)ccnc2C2CCC(F)(F)CC2)CC1]

To a solution of 2-(4,4-difluorocyclohexyl)-4-(2,5-difluorophenyl)pyridine-3-carboxylic acid (0.0500 g, 0.142 mmol) in dichloromethane (1 mL) was added N,N-diisopropylethylamine (0.0549 g, 0.425 mmol) and O-(7-Azabenzotriazol-1-yl)-N,N,N′,N′-tetramethyluronium hexafluorophosphate (0.0646 g, 0.170 mmol). The mixture was stirred at 50° C. for 12 h. The reaction mixture was cooled to ambient temperature and concentrated under reduced pressure. The residue was purified by preparative HPLC, eluting with a gradient of 35-65% of acetonitrile in water containing 0.225% formic acid, to afford the title compound as a colorless solid (0.0388 g, 58% yield): ¹H NMR (400 MHz, CDCl₃) δ 8.64 (d, J=4.8 Hz, 1H), 7.17-7.06 (m, 4H), 5.36 (d, J=8.0 Hz, 1H), 3.79 (dtd, J=14.8, 7.2, 3.6 Hz, 1H), 3.32 (s, 3H), 3.12-2.95 (m, 2H), 2.28-2.19 (m, 2H), 2.18-2.08 (m, 2H), 2.00-1.92 (m, 4H), 1.86-1.70 (m, 4H), 1.35-1.25 (m, 2H), 1.01-0.89 (m, 2H); MS (ES+) m/z 465.2 (M+1).

### Example 78

### Synthesis of N-(2-(4,4-difluorocyclohexyl)-4-(2,5-difluorophenyl)pyridin-3-yl)-2-(2-methylprop-1-en-1-yl)pyrimidine-5-carboxamide

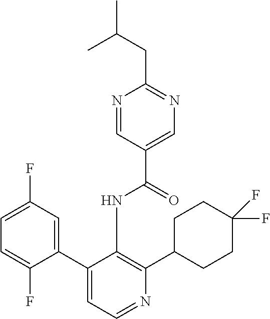 [SMILES: CC(C)Cc1ncc(C(=O)Nc2c(-c3cc(F)ccc3F)ccnc2C2CCC(F)(F)CC2)cn1]

### Step 1. Preparation of N-(2-(4,4-difluorocyclohexyl)-4-(2,5-difluorophenyl)pyridin-3-yl)-2-(2-methylprop-1-en-1-yl)pyrimidine-5-carboxamide

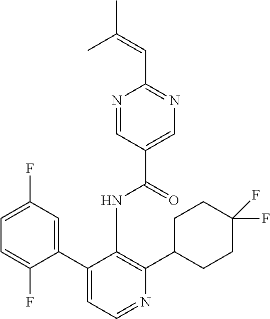 [SMILES: CC(C)=Cc1ncc(C(=O)Nc2c(-c3cc(F)ccc3F)ccnc2C2CCC(F)(F)CC2)cn1]

To a solution of 2-chloro-N-(2-(4,4-difluorocyclohexyl)-4-(2,5-difluorophenyl)pyridin-3-yl)pyrimidine-5-carboxamide (0.100 g, 0.215 mmol) and 4,4,5,5-tetramethyl-2-(2-methylprop-1-en-1-yl)-1,3,2-dioxaborolane (0.0588 g, 0.323 mmol) in dioxane (5 mL) and water (1 mL) was added potassium carbonate (0.0892 g, 0.645 mmol) and [1,1′-bis(diphenylphosphino)ferrocene]dichloropalladium(II) (0.0315 g, 0.0430 mmol). The mixture was stirred at 100° C. for 12 h under a nitrogen atmosphere. After cooling to ambient temperature, the mixture was concentrated under reduced pressure. The residue was diluted with ethyl acetate (20 mL) and water (20 mL). The layers were separated, and the aqueous phase was extracted with ethyl acetate (3×20 mL). The combined organic extracts were washed with brine (20 mL) and then dried over anhydrous sodium sulfate and filtered. The filtrate was concentrated under reduced pressure. The residue was purified by silica gel chromatography, eluting with 25% of ethyl acetate in petroleum ether, to afford the title compound as a colorless solid (0.0600 g, 54% yield): ¹H NMR (400 MHz, DMSO-d₆) δ 10.32 (s, 1H), 8.97 (s, 2H), 8.62 (d, J=4.8 Hz, 1H), 7.38 (d, J=5.2 Hz, 1H), 7.34 (dd, J=9.2, 4.8 Hz, 1H), 7.30-7.20 (m, 2H), 6.43 (s, 1H), 3.26-3.16 (m, 1H), 2.29 (s, 3H), 2.13-2.06 (m, 2H), 2.01 (s, 3H), 1.94-1.76 (m, 6H); MS (ES+) m/z 485.3 (M+1).

### Step 2. Preparation of N-(2-(4,4-difluorocyclohexyl)-4-(2,5-difluorophenyl)pyridin-3-yl)-2-(2-methylprop-1-en-1-yl)pyrimidine-5-carboxamide

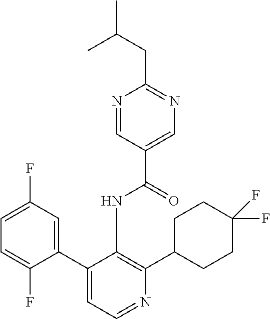 [SMILES: CC(C)Cc1ncc(C(=O)Nc2c(-c3cc(F)ccc3F)ccnc2C2CCC(F)(F)CC2)cn1]

To a solution of N-(2-(4,4-difluorocyclohexyl)-4-(2,5-difluorophenyl)pyridin-3-yl)-2-(2-methylprop-1-en-1-yl)pyrimidine-5-carboxamide (0.0600 g, 0.124 mmol) in methanol (5 mL) was added p palladium on active carbon (0.00600 g, 10 wt %) under a nitrogen atmosphere. The mixture was degassed and purged with hydrogen three times. The mixture was stirred at 25° C. for 3 h under a hydrogen atmosphere (15 psi). The resulting mixture was filtered over Celite, and the filtrate was concentrated under reduced pressure. The residue was purified by preparative HPLC, eluting with a gradient of 48-78% of acetonitrile in water containing 0.225% formic acid, to afford the title compound as an off-white solid (0.0215 g, 0.0438 mmol, 35% yield): ¹H NMR (400 MHz, DMSO-d₆) δ 10.39 (s, 1H), 8.94 (s, 2H), 8.62 (d, J=4.8 Hz, 1H), 8.46 (s, 1H), 7.37 (d, J=4.8 Hz, 1H), 7.33 (dd, J=9.2, 4.4 Hz, 1H), 7.30-7.20 (m, 2H), 3.23-3.17 (m, 1H), 2.80 (d, J=7.2 Hz, 2H), 2.22 (tt, J=13.6, 6.8 Hz, 1H), 2.08 (d, J=4.4 Hz, 2H), 2.03-1.81 (m, 6H), 0.91 (d, J=6.8 Hz, 6H); MS (ES+) m/z 487.3 (M+1).

### Example 79-125

In a similar manner as described in examples disclosed herein (e.g., Example 56 step 6), utilizing the appropriately substituted starting materials and intermediates, the following compounds were prepared:

| Example | Structure | | MS (ES+) | |
|---|---|---|---|---|
| No. | Name | Yield | m/z | ¹H-NMR |
| 79 | 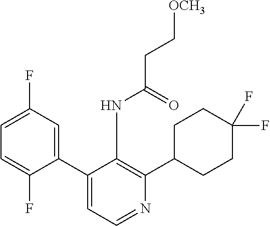 [SMILES: COCCC(=O)Nc1c(-c2cc(F)ccc2F)ccnc1C1CCC(F)(F)CC1] | 27% | 411.2 (M + 1) | (400 MHz, CDCl₃) δ 8.60 (d, J = 4.8 Hz, 1H), 7.94 (s, 1H), 7.20- 7.08 (m, 3H), 7.04 (ddd, J = 8.0, 5.6, 2.8 Hz, 1H), 3.52 (t, J = 5.4 Hz, 2H), 3.33 (s, 3H), 2.98 (t, J = 11.2 Hz, 1H), 2.47 (t, J = 5.6 Hz, 2H), 2.33-2.17 (m, 2H), 2.16-2.01 (m, 2H), 1.97-1.79 (m, 4H) |
| | N-(2-(4,4- | | | |
| | difluorocyclohexyl)-4-(2,5- | | | |
| | difluorophenyl)pyridin-3-yl)- | | | |
| | 3-methoxypropanamide | | | |
| 80 | 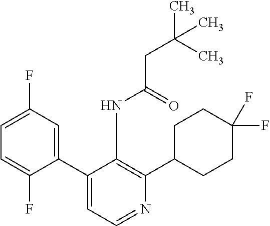 [SMILES: CC(C)(C)CC(=O)Nc1c(-c2cc(F)ccc2F)ccnc1C1CCC(F)(F)CC1] | 37% | 422.2 (M + 1) | (400 MHz; CDCl₃) δ 8.63 (d, J = 4.9 Hz, 1H), 7.17-7.11 (m, 2H), 7.10-7.05 (m, 1H), 6.80 (d, J = 0.8 Hz, 1H), 3.06-2.98 (m, 1H), 2.31- 2.23 (m, 2H), 2.19-2.08 (m, 4H), 1.95-1.73 (m, 4H), 0.95 (s, 9H) |
| | N-(2-(4,4- | | | |
| | difluorocyclohexyl)-4-(2,5- | | | |
| | difluorophenyl)pyridin-3-yl)- | | | |
| | 3,3-dimethylbutanamide | | | |
| 81 | 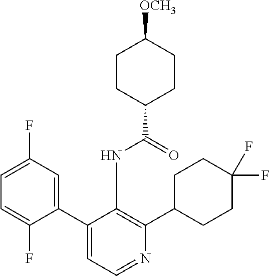 [SMILES: CO[C@H]1CC[C@H](C(=O)Nc2c(-c3cc(F)ccc3F)ccnc2C2CCC(F)(F)CC2)CC1] | 9% | 464.2 (M + 1) | (400 MHz; MeOD) δ 8.55 (d, J = 5.0 Hz, 1H), 7.30 (d, J = 4.9 Hz, 1H), 7.26-7.21 (m, 2H), 7.07 (s, 1H), 4.60 (s, 1H), 3.36-3.32 (s, 3H), 3.17-3.05 (m, 2H), 2.28- 2.15 (m, 2H), 2.15-1.96 (m, 4H), 1.91-1.82 (m, 3H), 1.75-1.27 (m, 3H), 1.33-1.13 (m, 4H) |
| | (1r,4r)-N-(2-(4,4- | | | |
| | difluorocyclohexyl)-4-(2,5- | | | |
| | difluorophenyl)pyridin-3-yl)- | | | |
| | 4-methoxycyclohexane-1- | | | |
| | carboxamide | | | |
| 82 | 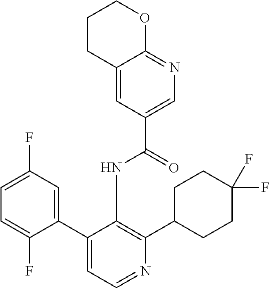 [SMILES: O=C(Nc1c(-c2cc(F)ccc2F)ccnc1C1CCC(F)(F)CC1)c1cnc2c(c1)CCCO2] | 11% | 486.3 (M + 1) | (400 MHz, DMSO-d₆) δ 10.02 (s, 1H), 8.60 (d, J = 4.8 Hz, 1H), 8.39 (d, J = 2.0 Hz, 1H), 7.83 (d, J = 2.0 Hz, 1H), 7.35 (d, J = 4.8 Hz, 1H), 7.31 (dd, J = 9.2, 4.4 Hz, 1H), 7.27-7.19 (m, 2H), 4.34-4.30 (m, 2H), 3.19-3.10 (m, 1H), 2.79 (t, J = 6.4 Hz, 2H), 2.10 (d, J = 4.4 Hz, 2H), 1.95-1.89 (m, 4H), 1.84 (s, 4H) |
| | N-(2-(4,4- | | | |
| | difluorocyclohexyl)-4-(2,5- | | | |
| | difluorophenyl)pyridin-3-yl)- | | | |
| | 3,4-dihydro-2H-pyrano[2,3- | | | |
| | b]pyridine-6-carboxamide | | | |
| 83 | 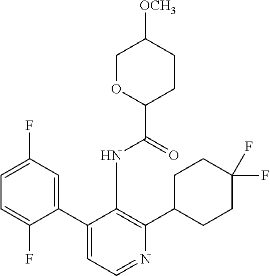 [SMILES: COC1CCC(C(=O)Nc2c(-c3cc(F)ccc3F)ccnc2C2CCC(F)(F)CC2)OC1] | 46% | 467.1 (M + 1) | ¹H NMR (400 MHz, MeOD-d₄) δ 8.54 (d, J = 5.2 Hz, 1H), 7.29 (d, J = 5.2 Hz, 1H), 7.27-7.16 (m, 2H), 7.10 (ddd, J = 8.4, 5.6, 2.8 Hz, 1H), 4.18-4.09 (m, 1H), 3.78 (dd, J = 11.2, 2.4 Hz, 1H), 3.36 (s, 3H), 3.28- 3.02 (m, 3H), 2.23-2.09 (m, 3H), 2.07-1.78 (m, 7H), 1.42-1.26 (m, 1H), 1.23-1.09 (m, 1H) |
| | N-(2-(4,4- | | | |
| | difluorocyclohexyl)-4-(2,5- | | | |
| | difluorophenyl)pyridin-3-yl)- | | | |
| | 5-methoxytetrahydro-2H- | | | |
| | pyran-2-carboxamide | | | |
| 84 | 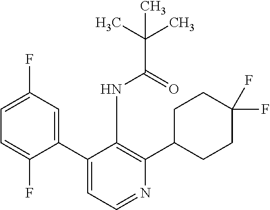 [SMILES: CC(C)(C)C(=O)Nc1c(-c2cc(F)ccc2F)ccnc1C1CCC(F)(F)CC1] | 50% | 409.2 (M + 1) | ¹H NMR (400 MHz, CDCl₃) δ 8.60 (d, J = 4.8 Hz, 1H), 7.16-7.07 (m, 3H), 7.07-6.99 (m, 2H), 2.90 (t, J = 11.2 Hz, 1H), 2.32-2.18 (m, 2H), 2.15-2.01 (m, 2H), 1.97-1.87 (m, 2H), 1.86-1.65 (m, 2H), 1.11 (s, 9H) |
| | N-(2-(4,4- | | | |
| | difluorocyclohexyl)-4-(2,5- | | | |
| | difluorophenyl)pyridin-3- | | | |
| | yl)pivalamide | | | |
| 85 | 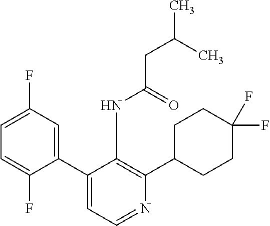 [SMILES: CC(C)CC(=O)Nc1c(-c2cc(F)ccc2F)ccnc1C1CCC(F)(F)CC1] | 12% | 409.2 (M + 1) | ¹H NMR (400 MHz, CDCl₃) δ 8.61 (d, J = 4.8 Hz, 1H), 7.17-7.07 (m, 3H), 7.07-7.01 (m, 1H), 6.80 (s, 1H), 2.98 (t, J = 11.6 Hz, 1H), 2.30-2.19 (m, 2H), 2.17-2.03 (m, 4H),2.00 (td, J = 6.8, 13.2 Hz, 1H), 1.95-1.85 (m, 2H), 1.84-1.69 (m, 2H), 0.82 (d, J = 6.4 Hz, 6H) |
| | N-(2-(4,4- | | | |
| | difluorocyclohexyl)-4-(2,5- | | | |
| | difluorophenyl)pyridin-3-yl)- | | | |
| | 3-methylbutanamide | | | |
| 86 | 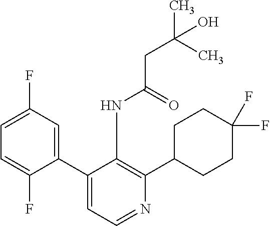 [SMILES: CC(C)(O)CC(=O)Nc1c(-c2cc(F)ccc2F)ccnc1C1CCC(F)(F)CC1] | 19% | 425.4 (M + 1) | ¹H NMR (400 MHz, MeOD-d₄) δ 8.54 (d, J = 4.8 Hz, 1H), 7.28 (d, J = 4.8 Hz, 1H), 7.24-7.16 (m, 2H), 7.12-7.05 (m, 1H), 3.20 (t, J = 11.6 Hz, 1H), 2.33 (s, 2H), 2.21-2.11 (m, 2H), 2.09-1.98 (m, 2H), 1.97-1.87 (m, 2H), 1.87-1.77 (m, 2H), 1.15 (s, 6H) |
| | N-(2-(4,4- | | | |
| | difluorocyclohexyl)-4-(2,5- | | | |
| | difluorophenyl)pyridin-3-yl)- | | | |
| | 3-hydroxy-3- | | | |
| | methylbutanamide | | | |
| 87 | 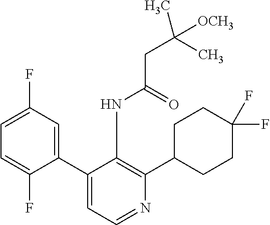 [SMILES: COC(C)(C)CC(=O)Nc1c(-c2cc(F)ccc2F)ccnc1C1CCC(F)(F)CC1] | 12% | 438.2 | ¹H-NMR (400 MHz; DMSO-d₆) δ 9.42 (s, 1H), 8.53 (d, J = 4.9 Hz, 1H), 7.39-7.29 (m, 2H), 7.27 (dd, J = 4.2, 0.7 Hz, 1H), 7.17 (ddd, J = 8.8, 5.7, 3.2 Hz, 1H), 3.15-3.11 (s, 1H), 3.10 (s, 3H), 2.32 (s, 2H), 2.19-2.10 (m, 2H), 1.90- 1.77 (m, 6H), 1.06 (s, 6H) |
| | N-(2-(4,4- | | (M + 1) | |
| | difluorocyclohexyl)-4-(2,5- | | | |
| | difluorophenyl)pyridin-3-yl)- | | | |
| | 3-methoxy-3- | | | |
| | methylbutanamide | | | |
| 88 | 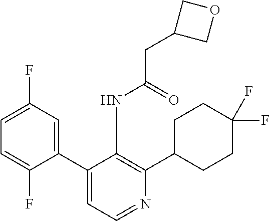 [SMILES: O=C(CC1COC1)Nc1c(-c2cc(F)ccc2F)ccnc1C1CCC(F)(F)CC1] | 7% | 422.2 (M + 1) | ¹H-NMR (400 MHz; CDCl₃) δ 8.64 (d, J = 4.9 Hz, 1H), 7.21-7.14 (m, 3H), 7.03 (ddd, J = 8.2, 5.6, 2.7 Hz, 1H), 6.84 (s, 1H), 4.77 (dd, J = 7.3, 6.6 Hz, 2H), 4.24 (t, J = 6.1 Hz, 2H), 3.29- 3.22 (m, 1H), 2.96-2.89 (m, 1H), 2.66 (d, J = 7.9 Hz, 2H), 2.32-2.22 (m, 2H), 2.18-2.06 (m, 2H), 1.92-1.72 (m, 4H) |
| | N-(2-(4,4- | | | |
| | difluorocyclohexyl)-4-(2,5- | | | |
| | difluorophenyl)pyridin-3-yl)- | | | |
| | 2-(oxetan-3-yl)acetamide | | | |
| 89 | 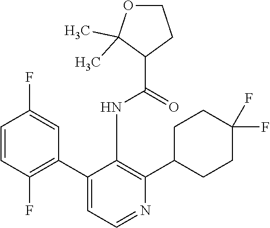 [SMILES: CC1(C)OCCC1C(=O)Nc1c(-c2cc(F)ccc2F)ccnc1C1CCC(F)(F)CC1] | 6% | 450.1 (M + 1) | ¹H-NMR (400 MHz; CDCl₃) δ 8.64 (d, J = 4.9 Hz, 1H), 7.19-7.13 (m, 3H), 7.06 (ddd, J = 8.1, 5.6, 2.7 Hz, 1H), 7.00 (broad singlet, 1H), 3.96 (td, J = 8.5, 5.1 Hz, 1H), 3.87 (q, J = 8.0 Hz, 1H), 3.00-2.94 (m, 1H), 2.59 (t, J = 8.0 Hz, 1H), 2.32-2.24 (m, 2H), 2.20- 2.08 (m, 4H), 1.95-1.90 (m, 2H), 1.89-1.71 (m, 2H), 1.28 (s, 3H), 0.98 |
| | N-(2-(4,4- | | | (s, 3H). |
| | difluorocyclohexyl)-4-(2,5- | | | |
| | difluorophenyl)pyridin-3-yl)-2,2- | | | |
| | dimethyltetrahydrofuran-3- | | | |
| | carboxamide | | | |
| 90 | 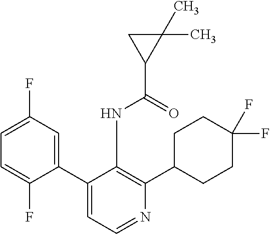 [SMILES: CC1(C)CC1C(=O)Nc1c(-c2cc(F)ccc2F)ccnc1C1CCC(F)(F)CC1] | 12% | 420.2 (M + 1) | ¹H-NMR (400 MHz; DMSO-d₆) δ 9.41 (s, 1H), 8.52 (d, J = 4.9 Hz, 1H), 7.34-7.27 (m, 2H), 7.26-7.23 (m, 1H), 7.15- 7.11 (m, 1H), 3.17-3.12 (m, 2H), 2.20-2.13 (m, 2H), 1.96-1.78 (m, 6H), 1.56 (dd, J = 7.0, 6.1 Hz, 1H), 1.09 (s, 3H), 0.88 (d, J = 6.8 Hz, 2H), 0.82 (t, J = 4.5 Hz, 1H), 0.69-0.66 (m, 1H). |
| | N-(2-(4,4- | | | |
| | difluorocyclohexyl)-4-(2,5- | | | |
| | difluorophenyl)pyridin-3-yl)- | | | |
| | 2,2-dimethylcyclopropane- | | | |
| | 1-carboxamide | | | |
| 91 | 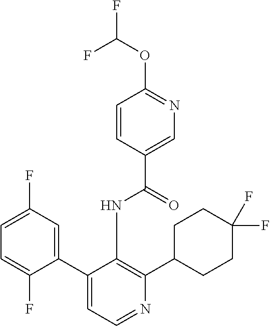 [SMILES: O=C(Nc1c(-c2cc(F)ccc2F)ccnc1C1CCC(F)(F)CC1)c1ccc(OC(F)F)nc1] | 56% | 496.2 (M + 1) | ¹H NMR (400 MHz, CDCl₃) δ 8.69 (d, J = 4.8 Hz, 1H), 8.52 (d, J = 2.4 Hz, 1H), 8.10 (dd, J = 8.8, 2.4 Hz, 1H), 7.70-7.31 (m, 2H), 7.22 (d, J = 4.8 Hz, 1H), 7.17-7.04 (m, 3H), 6.99 (d, J = 8.4 Hz, 1H), 3.06-2.94 (m, 1H), 2.30-2.20 (m, 2H), 2.19-2.09 (m, 2H), 1.95 (d, J = 13.6 Hz, 2H), 1.87-1.69 (m, 2H) |
| | 5-chloro-4′-(4- | | | |
| | methylpiperazin-1-yl)-[1,1′- | | | |
| | biphenyl]-2-ol | | | |
| 92 | 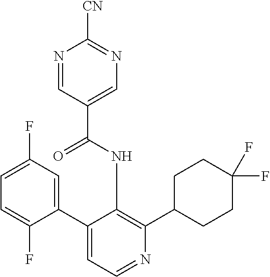 [SMILES: N#Cc1ncc(C(=O)Nc2c(-c3cc(F)ccc3F)ccnc2C2CCC(F)(F)CC2)cn1] | 62% | 455.9 (M + 1) | ¹H NMR (400 MHz, DMSO-d₆) δ 10.74 (br s, 1H), 9.20 (s, 2H), 8.64 (d, J = 4.8 Hz, 1H), 7.39 (d, J = 5.2 Hz, 1H), 7.36-7.21 (m, 3H), 3.23-3.16 (m, 1H), 2.12-1.79 (m, 8H) |
| | 2-cyano-N-(2-(4,4- | | | |
| | difluorocyclohexyl)-4-(2,5- | | | |
| | difluorophenyl)pyridin-3- | | | |
| | yl)pyrimidine-5- | | | |
| | carboxamide | | | |
| 93 | 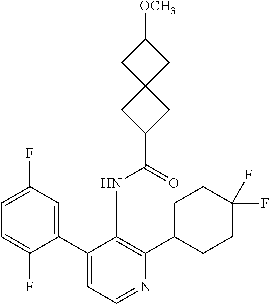 [SMILES: COC1CC2(C1)CC(C(=O)Nc1c(-c3cc(F)ccc3F)ccnc1C1CCC(F)(F)CC1)C2] | 18% | 477.2 (M + 1) | ¹H NMR (400 MHz, DMSO-d₆) δ 9.35 (s, 1H), 8.51 (d, J = 4.8 Hz, 1H), 7.38-7.28 (m, 2H), 7.26 (d, J = 4.8 Hz, 1H), 7.11 (ddd, J = 8.8, 5.6, 3.2 Hz, 1H), 3.64 (quin, J = 7.2 Hz, 1H), 3.06 (s, 3H), 3.04-2.93 (m, 2H), 2.29 (td, J = 10.8, 5.6 Hz, 1H), 2.20-2.06 (m, 2H), 2.05-1.98 (m, 2H), 1.96-1.71 (m, 10H), 1.61 (dd, J = 11.2, 7.2 Hz, 1H) |
| | N-(2-(4,4- | | | |
| | difluorocyclohexyl)-4-(2,5- | | | |
| | difluorophenyl) pyridin-3-yl)-6- | | | |
| | methoxyspiro[3.3]heptane- | | | |
| | 2-carboxamide | | | |
| 94 | 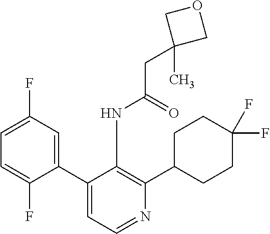 [SMILES: CC1(CC(=O)Nc2c(-c3cc(F)ccc3F)ccnc2C2CCC(F)(F)CC2)COC1] | 4% | 437.1 (M + 1) | ¹H NMR (400 MHz, CDCl₃) δ 8.63-8.62 (d, J = 4.0 Hz, 1H), 7.20- 7.12 (m, 3H), 7.06-7.02 (m, 1H), 6.85 (s, 1H), 4.48-4.47 (d, J = 4.0 Hz, 2H), 4.36-4.35 (d, J = 4.0 Hz, 2H), 2.99- 2.93 (m, 1H), 2.57 (s, 2H), 2.30-2.23 (m, 2H), 2.17-2.05 (m, 2H), 1.92-1.88 (m, 2H), 1.85-1.72 (m, 2H), 1.19 (s, 3H) |
| | N-(2-(4,4- | | | |
| | difluorocyclohexyl)-4-(2,5- | | | |
| | difluorophenyl)pyridin-3-yl)- | | | |
| | 2-(3-methyloxetan-3- | | | |
| | yl)acetamide | | | |
| 95 | 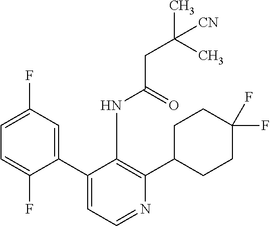 [SMILES: CC(C)(C#N)CC(=O)Nc1c(-c2cc(F)ccc2F)ccnc1C1CCC(F)(F)CC1] | 12% | 434.3 (M + 1) | ¹H NMR (400 MHz, DMSO-d₆) δ 8.69 (d, J = 4.8 Hz, 1H), 8.65 (s, 1H), 7.41 (d, J = 4.8 Hz, 1H), 7.39-7.33 (m, 2H), 7.03-6.93 (m, 1H), 2.78 (d, J = 18.0 Hz, 1H), 2.73-2.65 (m, 1H), 2.27 (d, J = 18.0 Hz, 1H), 2.08 (s, 2H), 1.99-1.85 (m, 3H), 1.81 (d, J = 7.2 Hz, 2H), 1.71 (d, J = 7.2 Hz, 1H), 1.31 (s, 3H), 0.89 (s, 3H) |
| | N-(2-(4,4- | | | |
| | difluorocyclohexyl)-4-(2,5- | | | |
| | difluorophenyl)pyridin-3-yl)- | | | |
| | 2-(oxetan-3-yl)acetamide | | | |
| 96 | 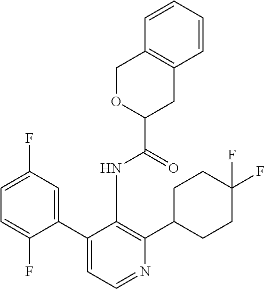 [SMILES: O=C(Nc1c(-c2cc(F)ccc2F)ccnc1C1CCC(F)(F)CC1)C1Cc2ccccc2CO1] | 58% | 485.1 (M + 1) | ¹H NMR (400 MHz, DMSO-d₆) δ 9.63 (s, 1H), 8.56 (d, J = 4.8 Hz, 1H), 7.43-7.27 (m, 3H), 7.25-7.16 (m, 3H), 7.15-7.11 (m, 1H), 7.10-7.03 (m, 1H), 4.84 (q, J = 15.2 Hz, 2H), 4.23 (dd, J = 10.4, 4.0 Hz, 1H), 3.10-2.98 (m, 1H), 2.84 (dd, J = 16.4, 4.0 Hz, 1H), 2.19-2.04 (m, 2H), 1.95-1.71 (m, 6H) |
| | N-(2-(4,4- | | | |
| | difluorocyclohexyl)-4-(2,5- | | | |
| | difluorophenyl)pyridin-3- | | | |
| | yl)isochromane-3- | | | |
| | carboxamide | | | |
| 97 | 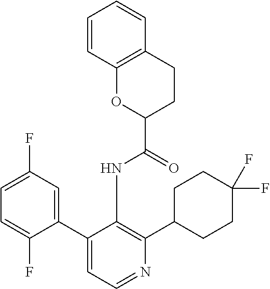 [SMILES: O=C(Nc1c(-c2cc(F)ccc2F)ccnc1C1CCC(F)(F)CC1)C1CCc2ccccc2O1] | 20% | 485.1 (M + 1) | ¹H NMR (400 MHz, CDCl₃) δ 8.77 (d, J = 5.2 Hz, 1H), 8.34 (s, 1H), 7.49-7.35 (m, 1H), 7.21-7.15 (m, 1H), 7.13-7.04 (m, 3H), 7.04-6.94 (m, 2H), 6.94-6.88 (m, 1H), 4.66-4.52 (m, 1H), 3.08-2.93 (m, 1H), 2.87-2.69 (m, 1H), 2.68-2.50 (m, 1H), 2.40-2.15 (m, 5H), 2.04-1.86 (m, 3H), 1.85-1.56 (m, 2H) |
| | N-(2-(4,4- | | | |
| | difluorocyclohexyl)-4-(2,5- | | | |
| | difluorophenyl)pyridin-3- | | | |
| | yl)isochromane-3- | | | |
| | carboxamide | | | |
| 98 | 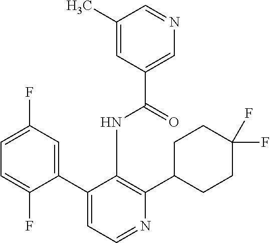 [SMILES: Cc1cncc(C(=O)Nc2c(-c3cc(F)ccc3F)ccnc2C2CCC(F)(F)CC2)c1] | 25% | 444.1 (M + 1) | ¹H NMR (400 MHz, DMSO-d₆) δ 10.22 (s, 1H), 8.68 (d, J = 1.6 Hz, 1H), 8.61 (d, J = 4.8 Hz, 1H), 8.56 (d, J = 1.2 Hz, 1H), 7.86 (s, 1H), 7.39- 7.29 (m, 2H), 7.28-7.18 (m, 2H), 3.22-3.09 (m, 1H), 2.34 (s, 3H), 2.18- 2.02 (m, 2H), 2.00-1.77 (m, 6H) |
| | N-(2-(4,4- | | | |
| | difluorocyclohexyl)-4-(2,5- | | | |
| | difluorophenyl)pyridin-3-yl)- | | | |
| | 5-methylnicotinamide | | | |
| 99 | 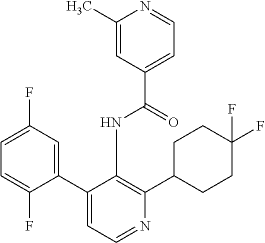 [SMILES: Cc1cc(C(=O)Nc2c(-c3cc(F)ccc3F)ccnc2C2CCC(F)(F)CC2)ccn1] | 31% | 444.2 (M + 1) | ¹H NMR (400 MHz, DMSO-d₆) δ 10.3 (s, 1H), 8.61 (dd, J = 31.2, 5.2 Hz, 2H), 7.47 (s, 1H), 7.44-7.39 (m, 1H), 7.39-7.30 (m, 2H), 7.30-7.19 (m, 2H), 3.23-3.10 (m, 1H), 2.53 (s, 3H), 2.18-2.05 (m, 2H), 2.03-1.75 (m, 6H) |
| | N-(2-(4,4- | | | |
| | difluorocyclohexyl)-4-(2,5- | | | |
| | difluorophenyl)pyridin-3-yl)- | | | |
| | 2-methylisonicotinamide | | | |
| 100 | 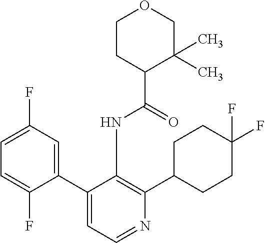 [SMILES: CC1(C)COCCC1C(=O)Nc1c(-c2cc(F)ccc2F)ccnc1C1CCC(F)(F)CC1] | 3% | 465.3 (M + 1) | ¹H NMR (400 MHz, CDCl₃) δ 8.68-8.60 (m, 1H), 7.21-7.03 (m, 4H), 6.88-6.74 (m, 1H), 4.05-3.94 (m, 1H), 3.49 (d, J = 11.2 Hz, 1H), 3.43-3.33 (m, 1H), 3.09 (d, J = 10.8 Hz, 1H), 3.05-2.93 (m, 1H), 2.35-2.22 (m, 2H), 2.21-2.04 (m, 3H), 1.97-1.73 (m, 4H), 1.38-1.26 (m, 2H), 0.99 (s, 3H), 0.91-0.72 (m, 3H) |
| | N-(2-(4,4- | | | |
| | difluorocyclohexyl)-4-(2,5- | | | |
| | difluorophenyl) pyridin-3-yl)- | | | |
| | 3,3-dimethyltetrahydro-2H- | | | |
| | pyran-4-carboxamide | | | |
| 101 | 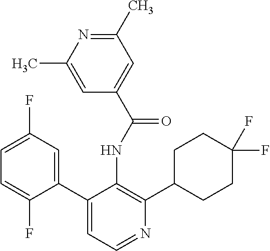 [SMILES: Cc1cc(C(=O)Nc2c(-c3cc(F)ccc3F)ccnc2C2CCC(F)(F)CC2)cc(C)n1] | 46% | 458.1 (M + 1) | ¹H NMR (400 MHz, CDCl₃) δ 8.69 (d, J = 4.8 Hz, 1H), 7.54-7.48 (m, 1H), 7.22 (d, J = 4.8 Hz, 1H), 7.16 (s, 2H), 7.15-7.04 (m, 3H), 2.99 (t, J = 11.2 Hz, 1H), 2.59 (s, 6H), 2.33-2.07 (m, 4H), 1.96 (d, J = 13.6 Hz, 2H), 1.89-1.69 (m, 2H) |
| | N-(2-(4,4- | | | |
| | difluorocyclohexyl)-4-(2,5- | | | |
| | difluorophenyl)-pyridin-3-yl)-2,6- | | | |
| | dimethylisonicotinamide | | | |
| 102 | 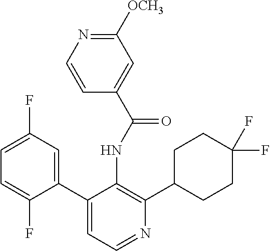 [SMILES: COc1cc(C(=O)Nc2c(-c3cc(F)ccc3F)ccnc2C2CCC(F)(F)CC2)ccn1] | 41% | 459.2 (M + 1) | ¹H-NMR (400 MHz; CDCl₃) δ 8.70 (d, J = 4.8 Hz, 1H), 8.30 (d, J = 5.2 Hz, 1H), 7.52 (singlet, 1H), 7.23 (d, J = 4.9 Hz, 1H), 7.17-7.07 (m, 4H), 6.96 (s, 1H), 3.99 (s, 3H), 3.02-2.96 (m, 1H), 2.29-2.20 (m, 2H), 2.20-2.10 (m, 2H), 1.98-1.92 (m, 2H), 1.88- 1.72 (m, 2H) |
| | N-(2-(4,4- | | | |
| | difluorocyclohexyl)-4-(2,5- | | | |
| | difluorophenyl)-pyridin-3-yl)-2- | | | |
| | methoxyisonicotinamide | | | |
| 103 | 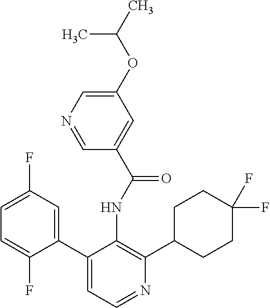 [SMILES: CC(C)Oc1cncc(C(=O)Nc2c(-c3cc(F)ccc3F)ccnc2C2CCC(F)(F)CC2)c1] | 16% | 488.2 (M + 1) | ¹H NMR (400 MHz, DMSO-d₆) δ 10.20 (s, 1H), 8.62 (d, J = 4.8 Hz, 1H), 8.43 (d, J = 1.6 Hz, 1H), 8.40 (d, J = 2.8 Hz, 1H), 7.50 (d, J = 1.6 Hz, 1H), 7.37 (d, J = 4.8 Hz, 1H), 7.33 (dt, J = 9.2, 4.4 Hz, 1H), 7.30-7.19 (m, 2H), 4.72 (dt, J = 12.0, 6.0 Hz, 1H), 3.25- 3.11 (m, 1H), 2.17-2.05 (m, 2H), 2.00-1.79 (m, 6H), 1.29 (d, J = 6.0 Hz, 6H) |
| | N-(2-(4,4- | | | |
| | difluorocyclohexyl)-4-(2,5- | | | |
| | difluorophenyl) pyridin-3-yl)- | | | |
| | 5-isopropoxynicotinamide | | | |
| 104 | 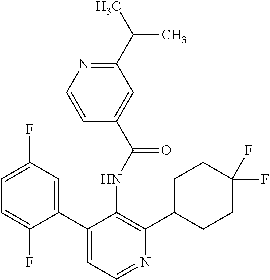 [SMILES: CC(C)c1cc(C(=O)Nc2c(-c3cc(F)ccc3F)ccnc2C2CCC(F)(F)CC2)ccn1] | 42% | 472.2 (M + 1) | ¹H NMR (400 MHz, DMSO-d₆) δ 10.29 (s, 1H), 8.63 (d, J = 4.8 Hz, 2H), 7.46-7.36 (m, 3H), 7.33 (dt, J = 9.2, 4.4 Hz, 1H), 7.30-7.19 (m, 2H), 3.22-3.12 (m, 1H), 3.07 (dt, J = 13.6, 6.8fa Hz, 1H), 2.19-2.14 (s, 2H), 2.01-1.76 (m, 6H), 1.24 (d, J = 6.8 Hz, 6H) |
| | N-(2-(4,4- | | | |
| | difluorocyclohexyl)-4-(2,5- | | | |
| | difluorophenyl) pyridin-3-yl)- | | | |
| | 2-isopropylisonicotinamide | | | |
| 105 | 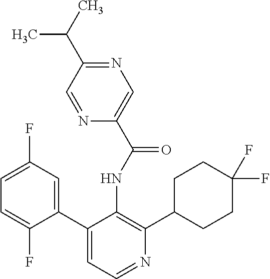 [SMILES: CC(C)c1cnc(C(=O)Nc2c(-c3cc(F)ccc3F)ccnc2C2CCC(F)(F)CC2)cn1] | 53% | 473.1 (M + 1) | ¹H NMR (400 MHz, DMSO-d₆) δ 10.51 (s, 1H), 9.00 (d, J = 1.2 Hz, 1H), 8.69 (d, J = 1.2 Hz, 1H), 8.60 (d, J = 4.8 Hz, 1H), 7.35 (dd, J = 5.2, 0.8 Hz, 1H), 7.33-7.26 (m, 2H), 7.25-7.18 (m, 1H), 3.23 (dt, J = 13.6, 6.8 Hz, 1H), 3.16-3.07 (m, 1H), 2.11-2.02 (m, 2H), 1.94-1.76 (m, 6H), 1.29 (d, J = 6.8 Hz, 6H) |
| | N-(2-(4,4- | | | |
| | difluorocyclohexyl)-4-(2,5- | | | |
| | difluorophenyl)pyridin-3-yl)- | | | |
| | 5-isopropylpyrazine-2- | | | |
| | carboxamide | | | |
| 106 | 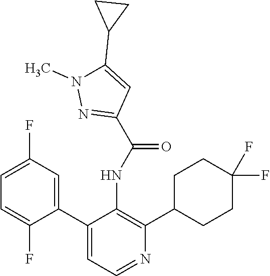 [SMILES: Cn1nc(C(=O)Nc2c(-c3cc(F)ccc3F)ccnc2C2CCC(F)(F)CC2)cc1C1CC1] | 10% | 473.0 (M + 1) | ¹H NMR (400 MHz, DMSO-d₆) δ 9.71 (s, 1H), 8.55 (d, J = 4.8 Hz, 1H), 7.34-7.27 (m, 2H), 7.27-7.19 (m, 2H), 6.24 (s, 1H), 3.89 (s, 3H), 3.12-3.04 (m, 1H), 2.15-2.05 (m, 2H), 1.93-1.80 (m, 6H), 1.79-1.70 (m, 1H), 1.00-0.89 (m, 2H), 0.65 (d, J = 5.2 Hz, 2H) |
| | 5-cyclopropyl-N-(2-(4,4- | | | |
| | difluorocyclohexyl)-4-(2,5- | | | |
| | difluorophenyl)pyridin-3-yl)- | | | |
| | 1-methyl-1H-pyrazole-3- | | | |
| | carboxamide | | | |
| 107 | 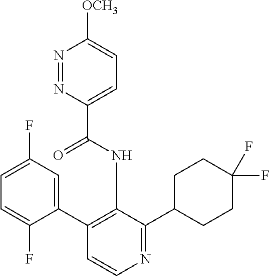 [SMILES: COc1ccc(C(=O)Nc2c(-c3cc(F)ccc3F)ccnc2C2CCC(F)(F)CC2)nn1] | 46% | 460.2 (M + 1) | ¹H-NMR (400 MHz; DMSO-d₆) δ 10.74 (s, 1H), 8.61 (d, J = 4.9 Hz, 1H), 8.00 (d, J = 9.1 Hz, 1H), 7.39-7.36 (m, 1H), 7.34-7.27 (m, 2H), 7.25- 7.20 (m, 1H), 4.13 (s, 3H), 3.20-3.13 (m, 1H), 2.13-2.04 (m, 2H), 1.93- 1.75 (m, 6H) |
| | N-(2-(4,4- | | | |
| | difluorocyclohexyl)-4-(2,5- | | | |
| | difluorophenyl)pyridin-3-yl)- | | | |
| | 6-methoxypyridazine-3- | | | |
| | carboxamide | | | |
| 108 | 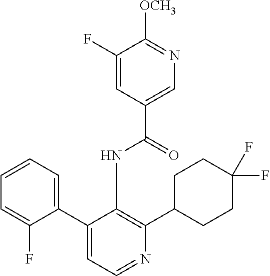 [SMILES: COc1ncc(C(=O)Nc2c(-c3ccccc3F)ccnc2C2CCC(F)(F)CC2)cc1F] | 57% | 460.1 (M + 1) | ¹H NMR (400 MHz, MeOD-d₄) δ 8.60 (d, J = 5.0 Hz, 1H), 8.33 (d, J = 2.0 Hz, 1H), 7.74 (dd, J = 10.6, 2.0 Hz, 1H), 7.45-7.38 (m, 2H), 7.36 (dt, J = 7.6, 2.0 Hz, 1H), 7.25-7.20 (m, 1H), 7.20-7.15 (m, 1H), 4.05 (s, 3H), 3.23-3.11 (m, 1H), 2.22-2.11 (m, 2H), 2.10-2.00 (m, 2H), 1.97-1.76 (m, 4H) |
| | N-(2-(4,4- | | | |
| | difluorocyclohexyl)-4-(2- | | | |
| | fluorophenyl)pyridin-3-yl)-5- | | | |
| | fluoro-6- | | | |
| | methoxynicotinamide | | | |
| 109 | 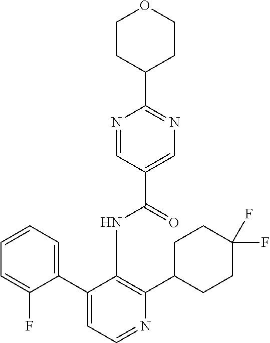 [SMILES: O=C(Nc1c(-c2ccccc2F)ccnc1C1CCC(F)(F)CC1)c1cnc(C2CCOCC2)nc1] | 7% | 497.3 (M + 1) | ¹H NMR (400 MHz, MeOD-d₄) δ 8.96 (s, 2H), 8.62 (d, J = 5.0 Hz, 1H), 7.49-7.35 (m, 3H), 7.28-7.17 (m, 2H), 4.10-4.00 (m, 2H), 3.70-3.55 (m, 2H), 3.26-3.12 (m, 2H), 2.24-2.03 (m, 4H), 2.02-1.84 (m, 8H) |
| | N-(2-(4,4- | | | |
| | difluorocyclohexyl)-4-(2- | | | |
| | fluorophenyl)pyridin-3-yl)-2- | | | |
| | (tetrahydro-2H-pyran-4-yl) | | | |
| | pyrimidine-5-carboxamide | | | |
| 110 | 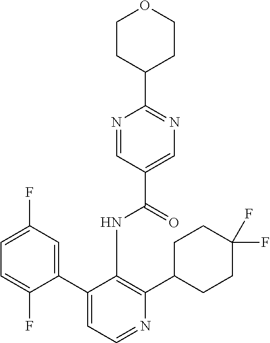 [SMILES: O=C(Nc1c(-c2cc(F)ccc2F)ccnc1C1CCC(F)(F)CC1)c1cnc(C2CCOCC2)nc1] | 53% | 515.2 (M + 1) | ¹H NMR (400 MHz, CDCl₃) δ 8.97 (s, 2H), 8.70 (d, J = 4.8 Hz, 1H), 7.64 (s, 1H), 7.22 (d, J = 4.8 Hz, 1H), 7.19-7.12 (m, 1H), 7.09 (td, J = 7.2, 4.4 Hz, 2H), 4.11 (dt, J = 11.2, 2.8 Hz, 2H), 3.58 (td, J = 11.2, 3.2 Hz, 2H), 3.21 (tt, J = 10.4, 4.8 Hz, 1H), 2.99 (t, J = 11.6 Hz, 1H), 2.29-2.21 (m, 2H), 2.19-2.09 (m, 2H), 2.03-1.93 (m, 6H), 1.88-1.73 (m, 2H) |
| | N-(2-(4,4- | | | |
| | difluorocyclohexyl)-4-(2,5- | | | |
| | difluorophenyl)pyridin-3-yl)- | | | |
| | 2-(tetrahydro-2H-pyran-4- | | | |
| | yl)pyrimidine-5- | | | |
| | carboxamide | | | |
| 111 | 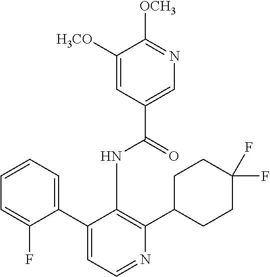 [SMILES: COc1cc(C(=O)Nc2c(-c3ccccc3F)ccnc2C2CCC(F)(F)CC2)cnc1OC] | 31% | 472.1 (M + 1) | ¹H NMR (400 MHz, DMSO-d₆) δ 10.03 (s, 1H), 8.58 (d, J = 4.8 Hz, 1H), 8.12 (d, J = 2.0 Hz, 1H), 7.43 (d, J = 1.6 Hz, 1H), 7.41-7.31 (m, 3H), 7.30-7.24 (m, 1H), 7.23-7.17 (m, 1H), 3.90 (s, 3H), 3.79 (s, 3H), 3.19-3.09 (m, 1H), 2.17-2.05 (m, 2H), 1.98-1.75 (m, 6H) |
| | N-(2-(4,4- | | | |
| | difluorocyclohexyl)-4-(2- | | | |
| | fluorophenyl)pyridin-3-yl)- | | | |
| | 5,6-dimethoxynicotinamide | | | |
| 112 | 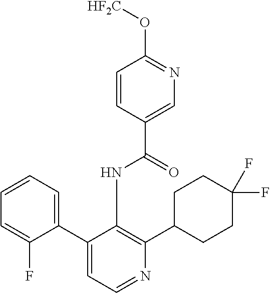 | 29% | 478.1 (M + 1) | ¹H NMR (400 MHz, DMSO-d₆) δ 10.20 (s, 1H), 8.61 (d, J = 4.8 Hz, 1H), 8.56 (d, J = 2.0 Hz, 1H), 8.16 (dd, J = 8.4, 2.4 Hz, 1H), 7.76 (t, J = 72.4 Hz, 1H), 7.45-7.32 (m, 3H), 7.30-7.24 (m, 1H), 7.24-7.15 (m, 2H), 3.21-3.10 (m, 1H), 2.10 (s, 2H), 2.01-1.76 (m, 6H) |
| | N-(2-(4,4- | | | |
| | difluorocyclohexyl)-4-(2- | | | |
| | fluorophenyl)pyridin-3-yl)-6- | | | |
| | (difluoromethoxy)nicotinamide | | | |
| 113 | 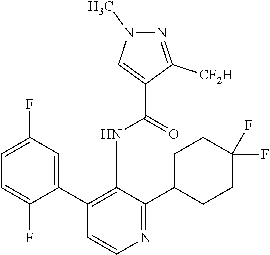 [SMILES: Cn1cc(C(=O)Nc2c(-c3cc(F)ccc3F)ccnc2C2CCC(F)(F)CC2)c(C(F)F)n1] | 9.0% | 483.0 (M + 1) | ¹H NMR (400 MHz, DMSO-d₆) δ 9.85-9.79 (m, 1H), 8.59 (d, J = 4.4 Hz, 1H), 8.23 (s, 1H), 7.34 (s, 1H), 7.33-6.86 (m, 4H), 3.93 (s, 3H), 3.19-3.06 (m, 1H), 2.12-2.09 (m, 2H), 1.96-1.88 (m, 2H), 1.87-1.77 (m, 4H) |
| | N-(2-(4,4- | | | |
| | difluorocyclohexyl)-4-(2,5- | | | |
| | difluorophenyl)pyridin-3-yl)- | | | |
| | 3-(difluoromethyl)-1-methyl- | | | |
| | 1H-pyrazole-4-carboxamide | | | |
| 114 | 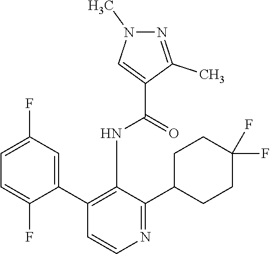 [SMILES: Cc1nn(C)cc1C(=O)Nc1c(-c2cc(F)ccc2F)ccnc1C1CCC(F)(F)CC1] | 12% | 447.0 (M + 1) | ¹H-NMR (400 MHz; DMSO-d₆) δ 9.40 (s, 1H), 8.57 (d, J = 4.6 Hz, 1H), 8.04 (s, 1H), 7.36- 7.31 (m, 2H), 7.31-7.25 (m, 1H), 7.22-7.17 (m, 1H), 3.78 (s, 3H), 3.17- 3.14 (m, 1H), 2.14 (s, 3H), 2.14-2.07 (m, 2H) 1.92-1.81 (m, 6H) |
| | N-(2-(4,4- | | | |
| | difluorocyclohexyl)-4-(2,5- | | | |
| | difluorophenyl)pyridin-3-yl)- | | | |
| | 1,3-dimethyl-1H-pyrazole-4- | | | |
| | carboxamide | | | |
| 115 | 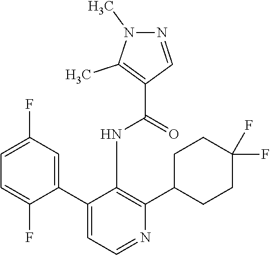 [SMILES: Cc1c(C(=O)Nc2c(-c3cc(F)ccc3F)ccnc2C2CCC(F)(F)CC2)cnn1C] | 22% | 447.0 (M + 1) | ¹H NMR (400 MHz, DMSO-d₆) δ 9.47 (s, 1H), 8.56 (d, J = 4.8 Hz, 1H), 7.78 (s, 1H), 7.35- 7.28 (m, 2H), 7.22 (dtd, J = 11.2, 8.4, 3.6 Hz, 2H), 3.70 (s, 3H), 3.18- 3.07 (m, 1H), 2.32 (s, 3H), 2.17-2.05 (m, 2H), 1.95-1.73 (m, 6H) |
| | N-(2-(4,4- | | | |
| | difluorocyclohexyl)-4-(2,5- | | | |
| | difluorophenyl)pyridin-3-yl)- | | | |
| | 1,5-dimethyl-1H-pyrazole-4- | | | |
| | carboxamide | | | |
| 116 | 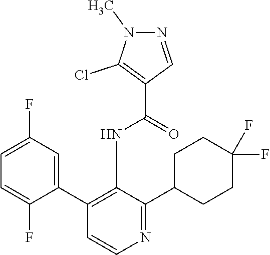 [SMILES: Cn1ncc(C(=O)Nc2c(-c3cc(F)ccc3F)ccnc2C2CCC(F)(F)CC2)c1Cl] | 9% | 467.2 (M + 1), 469.2 (M + 1) | ¹H NMR (400 MHz, DMSO-d₆) δ 9.69 (s, 1H), 8.59 (d, J = 4.4 Hz, 1H), 7.92 (s, 1H), 7.36- 7.30 (m, 2H), 7.27 (dd, J = 3.2, 8.0 Hz, 1H), 7.24-7.17 (m, 1H), 3.79 (s, 3H), 3.22-3.03 (m, 1H), 2.15-2.06 (m, 2H), 1.96-1.78 (m, 6H) |
| | 5-chloro-N-(2-(4,4- | | | |
| | difluorocyclohexyl)-4-(2,5- | | | |
| | difluorophenyl)pyridin-3-yl)- | | | |
| | 1-methyl-1H-pyrazole-4- | | | |
| | carboxamide | | | |
| 117 | 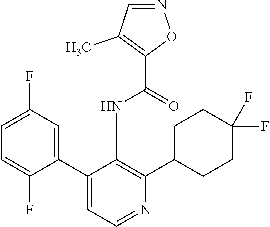 [SMILES: Cc1cnoc1C(=O)Nc1c(-c2cc(F)ccc2F)ccnc1C1CCC(F)(F)CC1] | 46% | 434.0 (M + 1) | ¹H NMR (400 MHz, DMSO-d₆) δ 10.51 (s, 1H), 8.65-8.60 (m, 2H), 7.36 (d, J = 4.4 Hz, 1H), 7.33 (dd, J = 9.2, 4.8 Hz, 1H), 7.29-7.20 (m, 2H), 3.19-3.09 (m, 1H), 2.17-2.10 (m, 2H), 2.08 (s, 3H), 1.95-1.81 (m, 6H) |
| | N-(2-(4,4- | | | |
| | difluorocyclohexyl)-4-(2,5- | | | |
| | difluorophenyl)pyridin-3-yl)- | | | |
| | 4-methylisoxazole-5- | | | |
| | carboxamide | | | |
| 118 | 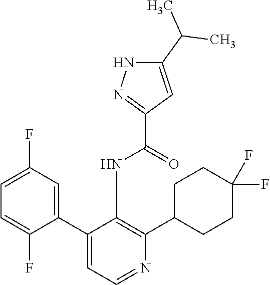 [SMILES: CC(C)c1cc(C(=O)Nc2c(-c3cc(F)ccc3F)ccnc2C2CCC(F)(F)CC2)n[nH]1] | 11% | 461.2 (M + 1) | ¹H NMR (400 MHz, CDCl₃) δ 8.65 (d, J = 4.8 Hz, 1H), 8.33 (s, 1H), 7.20 (d, J = 4.8 Hz, 1H), 7.13-7.00 (m, 3H), 6.59 (s, 1H), 3.15-2.96 (m, 2H), 2.29-2.18 (m, 2H), 2.17-2.08 (m, 2H), 2.03-1.95 (m, 2H), 1.90-1.80 (m, 2H), 1.33 (d, J = 6.8 Hz, 6H) |
| | N-(2-(4,4- | | | |
| | difluorocyclohexyl)-4-(2,5- | | | |
| | difluorophenyl)pyridin-3-yl)- | | | |
| | 5-isopropyl-1H-pyrazole-3- | | | |
| | carboxamide | | | |
| 119 | 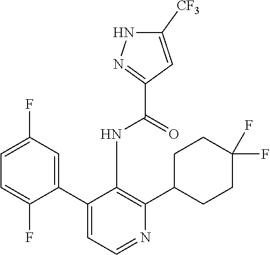 [SMILES: O=C(Nc1c(-c2cc(F)ccc2F)ccnc1C1CCC(F)(F)CC1)c1cc(C(F)(F)F)[nH]n1] | 9% | 487.1 (M + 1) | ¹H NMR (400 MHz, CDCl₃) δ 8.71 (d, J = 4.8 Hz, 1H), 7.97-7.77 (m, 1H), 7.23 (d, J = 4.8 Hz, 1H), 7.14-7.05 (m, 3H), 6.99 (s, 1H), 3.06-2.96 (m, 1H), 2.32-2.10 (m, 4H), 2.00-1.91 (m, 2H), 1.90-1.73 (m, 2H) |
| | N-(2-(4,4- | | | |
| | difluorocyclohexyl)-4-(2,5- | | | |
| | difluorophenyl)pyridin-3-yl)- | | | |
| | 5-(trifluoromethyl)-1H- | | | |
| | pyrazole-3-carboxamide | | | |
| 120 | 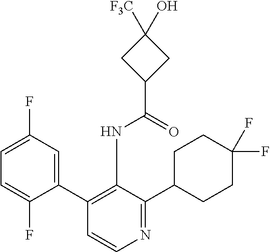 [SMILES: O=C(Nc1c(-c2cc(F)ccc2F)ccnc1C1CCC(F)(F)CC1)C1CC(O)(C(F)(F)F)C1] | 17% | 491.1 (M + 1) | ¹H NMR (400 MHz, MeOD) δ 8.54 (d, J = 4.8 Hz, 1H), 7.29 (d, J = 4.8 Hz, 1H), 7.27-7.13 (m, 2H), 7.07 (ddd, J = 2.8, 5.6, 8.4 Hz, 1H), 3.14-3.00 (m, 1H), 2.90 (quin, J = 8.8 Hz, 1H), 2.51 (t, J = 10.0 Hz, 2H), 2.29-2.10 (m, 3H), 2.09-1.94 (m, 3H), 1.94-1.70 (m, 4H) |
| | N-(2-(4,4- | | | |
| | difluorocyclohexyl)-4-(2,5- | | | |
| | difluorophenyl)pyridin-3-yl)- | | | |
| | 3-hydroxy-3-(trifluoromethyl) | | | |
| | cyclobutane-1-carboxamide | | | |
| 121 | 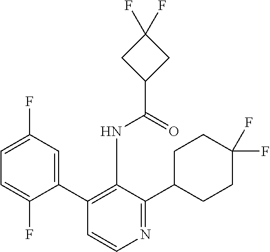 [SMILES: O=C(Nc1c(-c2cc(F)ccc2F)ccnc1C1CCC(F)(F)CC1)C1CC(F)(F)C1] | 53% | 443.0 (M + 1) | ¹H NMR (400 MHz, CDCl₃) δ 8.64 (d, J = 4.8 Hz, 1H), 7.22-7.10 (m, 3H), 7.08-6.99 (m, 1H), 6.84 (s, 1H), 2.98-2.88 (m, 1H), 2.87-2.79 (m, 1H), 2.73-2.50 (m, 4H), 2.34-2.19 (m, 2H), 2.18-2.04 (m, 2H), 1.93-1.73 (m, 4H) |
| | N-(2-(4,4- | | | |
| | difluorocyclohexyl)-4-(2,5- | | | |
| | difluorophenyl)pyridin-3-yl)- | | | |
| | 3,3-difluorocyclobutane-1- | | | |
| | carboxamide | | | |
| 122 | 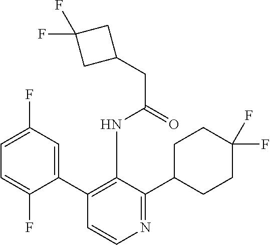 [SMILES: O=C(CC1CC(F)(F)C1)Nc1c(-c2cc(F)ccc2F)ccnc1C1CCC(F)(F)CC1] | 24% | 457.1 (M + 1) | ¹H NMR (400 MHz, CDCl₃) δ 8.63 (d, J = 4.8 Hz, 1H), 7.21-7.11 (m, 3H), 7.09-6.96 (m, 1H), 6.81 (s, 1H), 3.01- 2.86 (m, 1H), 2.70-2.54 (m, 2H), 2.54-2.34 (m, 3H), 2.33-2.20 (m, 2H), 2.18-1.96 (m, 4H), 1.89 (d, J = 15.2 Hz, 2H), 1.85-1.70 (m, 2H) |
| | 2-(3,3-difluorocyclobutyl)-N- | | | |
| | (2-(4,4-difluorocyclohexyl)-4-(2,5- | | | |
| | difluorophenyl)pyridin-3- | | | |
| | yl)acetamide | | | |
| 123 | 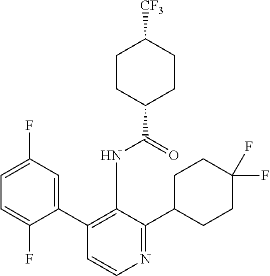 [SMILES: O=C(Nc1c(-c2cc(F)ccc2F)ccnc1C1CCC(F)(F)CC1)[C@H]1CC[C@@H](C(F)(F)F)CC1] | 16% | 503.1 (M + 1) | ¹H NMR (400 MHz, CDCl₃) δ 8.61 (d, J = 4.8 Hz, 1H), 7.18-7.07 (m, 3H), 7.06-6.98 (m, 1H), 6.85-6.75 (m, 1H), 2.98-2.85 (m, 1H), 2.32-2.19 (m, 2H), 2.18-1.95 (m, 6H), 1.94-1.86 (m, 2H), 1.84-1.68 (m, 4H), 1.43-1.27 (m, 4H) |
| | (1r,4r)-N-(2-(4,4- | | | |
| | difluorocyclohexyl)-4-(2,5- | | | |
| | difluorophenyl)pyridin-3-yl)-4- | | | |
| | (trifluoromethyl)cyclohexane-1- | | | |
| | carboxamide | | | |
| 124 | 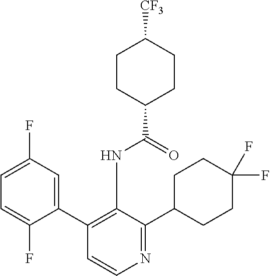 [SMILES: O=C(Nc1c(-c2cc(F)ccc2F)ccnc1C1CCC(F)(F)CC1)[C@H]1CC[C@@H](C(F)(F)F)CC1] | 27% | 503.1 (M + 1) | ¹H NMR (400 MHz, CDCl₃) δ 8.64 (d, J = 4.8 Hz, 1H), 7.20-7.11 (m, 3H), 7.07-7.05 (m, 1H), 7.00-6.91 (m, 1H), 3.04-2.82 (m, 1H), 2.54-2.40 (m, 1H), 2.33-2.21 (m, 2H), 2.19-2.10 (m, 2H), 2.08-2.02 (m, 1H), 1.99-1.89 (m, 4H), 1.88-1.75 (m, 2H), 1.74-1.65 (m, 3H), 1.56-1.43 (m, 3H) |
| | (1r,3r)-N-(2-(4,4- | | | |
| | difluorocyclohexyl)-4-(2,5- | | | |
| | difluorophenyl)pyridin-3-yl)-3- | | | |
| | (trifluoromethyl)cyclobutane-1- | | | |
| | carboxamide | | | |
| 125 | 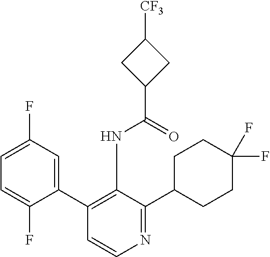 [SMILES: O=C(Nc1c(-c2cc(F)ccc2F)ccnc1C1CCC(F)(F)CC1)C1CC(C(F)(F)F)C1] | 32% | 475.1 (M + 1) | ¹H NMR (400 MHz, MeOD) δ 8.59-8.51 (m, 1H), 7.33-7.26 (m, 1H), 7.22 (qd, J = 4.8, 7.6 Hz, 2H), 7.13-7.01 (m, 1H), 3.23-2.88 (m, 3H), 2.34-2.11 (m, 5H), 1.92-1.73 (m, 4H) |
| | N-(2-(4,4- | | | |
| | difluorocyclohexyl)-4-(2,5- | | | |
| | difluorophenyl)pyridin-3-yl)-3- | | | |
| | (trifluoromethyl)cyclobutane-1- | | | |
| | carboxamide | | | |

### Example 126 and Example 127

### Synthesis of N-(2-(4,4-difluorocyclohexyl)-4-(2,5-difluorophenyl)pyridin-3-yl)-3-(trifluoromethyl)cyclobutane-1-carboxamide P1 and P2

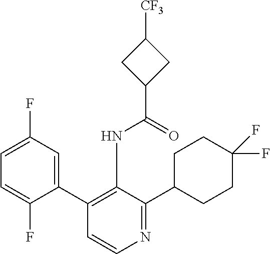 [SMILES: O=C(Nc1c(-c2cc(F)ccc2F)ccnc1C1CCC(F)(F)CC1)C1CC(C(F)(F)F)C1]

N-(2-(4,4-difluorocyclohexyl)-4-(2,5-difluorophenyl)pyridin-3-yl)-3-(trifluoromethyl)cyclobutane-1-carboxamide (0.0500 g, 0.0980 mmol) was purified by chiral SFC (column: DAICEL CHIRALPAK IG (250 mm×30 mm, 5 μm), eluting with 20% of isopropanol containing 0.1% ammonium hydroxide in supercritical carbon dioxide, to afford peak 1 (retention time=4.05 min) as a colorless solid (0.0128 g, 25% yield, 96% ee): ¹H NMR (400 MHz, MeOD) δ 8.55 (d, J=4.8 Hz, 1H), 7.29 (d, J=5.2 Hz, 1H), 7.27-7.14 (m, 2H), 7.14-7.00 (m, 1H), 3.27-3.16 (m, 1H), 3.14-2.99 (m, 1H), 2.96-2.77 (m, 1H), 2.53-2.09 (m, 5H), 2.09-1.93 (m, 3H), 1.92-1.69 (m, 4H); MS (ES+) m/z 475.1 (M+1).

Peak 2 (retention time=4.96 min) was isolated as a colorless solid (0.0286 g, 55% yield, 96% ee): ¹H NMR (400 MHz, MeOD) δ 8.54 (d, J=4.8 Hz, 1H), 7.28 (d, J=4.8 Hz, 1H), 7.25-7.15 (m, 2H), 7.12-7.01 (m, 1H), 3.20-2.90 (m, 3H), 2.31-2.09 (m, 5H), 2.09-1.96 (m, 3H), 1.93-1.77 (m, 4H); MS (ES+) m/z 475.1 (M+1).

### Example 128

### Synthesis of N-(2-(4,4-difluorocyclohexyl)-4-(2,5-difluorophenyl)pyridin-3-yl)-2-(2-methylprop-1-en-1-yl)pyrimidine-5-carboxamide

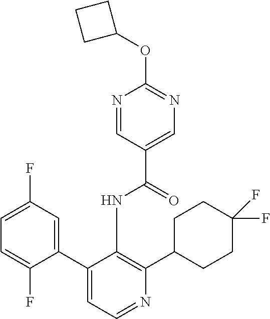 [SMILES: O=C(Nc1c(-c2cc(F)ccc2F)ccnc1C1CCC(F)(F)CC1)c1cnc(OC2CCC2)nc1]

To a solution of 2-chloro-N-(2-(4,4-difluorocyclohexyl)-4-(2,5-difluorophenyl)pyridin-3-yl)pyrimidine-5-carboxamide (0.0500 g, 0.108 mmol) in cyclobutanol (1 mL) was added cesium carbonate (0.105 g, 0.323 mmol). The mixture was stirred at 70° C. for 12 h. The mixture was cooled to ambient temperature and water (10 mL) was added. The mixture was extracted with ethyl acetate (3×20 mL). The combined organic layers were washed with brine (3×10 mL), dried over sodium sulfate, filtered, and concentrated under reduced pressure. The residue was purified by preparative HPLC, eluting with a gradient of 40-70% of acetonitrile in water containing 0.225% formic acid, to afford the title compound as a colorless solid (0.0267 g, 48% yield): ¹H NMR (400 MHz, CDCl₃) δ 8.82 (s, 2H), 8.69 (d, J=4.8 Hz, 1H), 7.48 (s, 1H), 7.21 (d, J=4.8 Hz, 1H), 7.18-7.01 (m, 3H), 5.25 (q, J=7.2 Hz, 1H), 2.99 (t, J=11.6 Hz, 1H), 2.56-2.41 (m, 2H), 2.29-2.08 (m, 6H), 2.00-1.82 (m, 4H), 1.75-1.68 (m, 2H); MS (ES+) m/z 501.3 (M+1).

### Example 129

### Synthesis of (S)—N-(2-(4,4-difluorocyclohexyl)-4-(2,5-difluorophenyl)pyridin-3-yl)-2-((1-methoxypropan-2-yl)oxy)pyrimidine-5-carboxamide

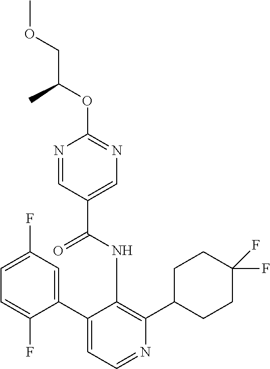 [SMILES: COC[C@H](C)Oc1ncc(C(=O)Nc2c(-c3cc(F)ccc3F)ccnc2C2CCC(F)(F)CC2)cn1]

To a solution of 2-chloro-N-(2-(4,4-difluorocyclohexyl)-4-(2,5-difluorophenyl)pyridin-3-yl)pyrimidine-5-carboxamide (0.0500 g, 0.108 mmol) in dimethylformamide (0.5 mL) and tetrahydrofuran (0.5 mL) was added cesium carbonate (0.105 g, 0.323 mmol), 1,4-diazabicyclo[2.2.2]octane (0.00241 g, 0.0215 mmol) and (S)-1-methoxypropan-2-ol (0.0485 g, 0.538 mmol). The mixture was stirred at 50° C. for 12 h. The reaction mixture was cooled to ambient temperature and concentrated under reduced pressure. The residue was purified by preparative HPLC, eluting with a gradient of 35-65% of acetonitrile in water containing 0.225% formic acid, to afford the title compound as a colorless solid (0.0151 g, 27% yield): ¹H NMR (400 MHz, CDCl₃) δ 8.83 (s, 2H), 8.69 (d, J=4.8 Hz, 1H), 7.42 (s, 1H), 7.21 (d, J=4.8 Hz, 1H), 7.17-7.05 (m, 3H), 5.54-5.43 (m, 1H), 3.69-3.63 (m, 1H), 3.59-3.52 (m, 1H), 3.40 (s, 3H), 3.07-2.93 (m, 1H), 2.28-2.20 (m, 2H), 2.19-2.08 (m, 2H), 1.95 (d, J=13.2 Hz, 2H), 1.87-1.71 (m, 2H), 1.40 (d, J=6.4 Hz, 3H); MS (ES+) m/z 519.3 (M+1).

### Example 130-154

In a similar manner as described in Example 129, utilizing the appropriately substituted starting materials and intermediates, the following compounds were prepared:

| Example | | Yield | MS (ES+) | |
|---|---|---|---|---|
| No. | Structure | | m/z | ¹H-NMR |
| 130 | 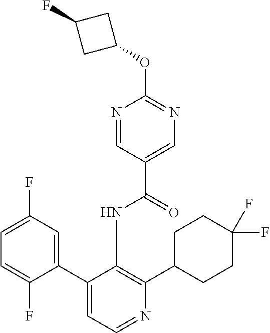 [SMILES: O=C(Nc1c(-c2cc(F)ccc2F)ccnc1C1CCC(F)(F)CC1)c1cnc(O[C@H]2C[C@H](F)C2)nc1] | 46% | 519.2 (M + 1) | (400 MHz, MeOD- d₄) δ 8.86 (s, 2H), 8.61 (d, J = 5.2 Hz, 1H), 8.52 (d, J = 5.2 Hz, 1H), 7.36 (d, J = 5.2 Hz, 1H), 7.25- 7.08 (m, 3H), 5.50 (dq, J = 4.4, 7.2 Hz, 1H), 5.41-5.17 (m, 1H), 3.23-3.10 (m, 1H), 2.84-2.67 (m, 2H), 2.65-2.47 (m, 2H), 2.23-1.97 (m, 4H), 1.96-1.72 (m, 4H) |
| | trans-N-(2-(4,4- | | | |
| | difluorocyclohexyl)-4-(2,5- | | | |
| | difluorophenyl)pyridin-3-yl)-2- | | | |
| | ((1r,3r)-3-fluorocyclobutoxy) | | | |
| | pyrimidine-5-carboxamide | | | |
| 131 | 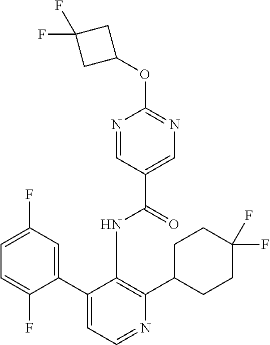 [SMILES: O=C(Nc1c(-c2cc(F)ccc2F)ccnc1C1CCC(F)(F)CC1)c1cnc(OC2CC(F)(F)C2)nc1] | 56% | 536.2 (M + 1) | (400 MHz; MeOD) δ 8.90 (s, 2H), 8.63 (d, J = 4.9 Hz, 1H), 7.38 (d, J = 4.9 Hz, 1H), 7.24-7.14 (m, 3H), 5.26-5.23 (m, 1H), 3.22-3.14 (m, 3H), 2.88-2.75 (m, 2H), 2.20-2.02 (m, 4H), 1.94-1.80 (m, 4H) |
| | 2-(3,3-difluorocyclobutoxy)- | | | |
| | N-(2-(4,4- | | | |
| | difluorocyclohexyl)-4-(2,5- | | | |
| | difluorophenyl)pyridin-3- | | | |
| | yl)pyrimidine-5-carboxamide | | | |
| 132 | 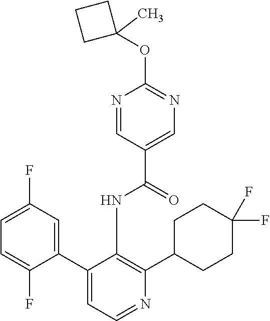 [SMILES: CC1(Oc2ncc(C(=O)Nc3c(-c4cc(F)ccc4F)ccnc3C3CCC(F)(F)CC3)cn2)CCC1] | 5% | 515.2 (M + 1) | (400 MHz; MeOD) δ 8.83 (s, 2H), 8.67 (dd, J = 2.4, 5.2 Hz, 1H), 7.59- 7.49 (m, 1H), 7.30-7.11 (m, 3H), 3.22-3.21 (m, 1H), 2.53-2.38 (m, 2H), 2.31 (tdd, J = 10.0, 7.6, 2.4 Hz, 2H), 2.23-2.11 (m, 2H), 2.09-2.00 (m, 2H), 1.97-1.95 (m, 3H), 1.90-1.81 (m, 2H), 1.80-1.72 (m, 1H), 1.70 (s, 3H) |
| | N-(2-(4,4- | | | |
| | difluorocyclohexyl)-4-(2,5- | | | |
| | difluorophenyl)pyridin-3-yl)-2-(1- | | | |
| | methylcyclobutoxy)pyrimidine-5- | | | |
| | carboxamide | | | |
| 133 | 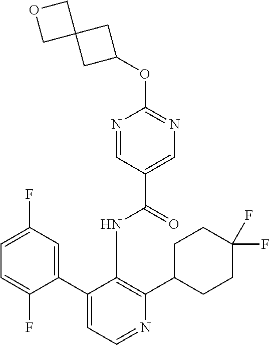 [SMILES: O=C(Nc1c(-c2cc(F)ccc2F)ccnc1C1CCC(F)(F)CC1)c1cnc(OC2CC3(COC3)C2)nc1] | 22% | 543.4 (M + 1) | (400 MHz, DMSO- d₆) δ 10.24 (s, 1H), 8.85 (s, 2H), 8.62 (d, J = 4.8 Hz, 1H), 8.46 (s, 1H), 7.40-7.31 (m, 2H), 7.31-7.21 (m, 2H), 5.04 (quin, J = 7.2 Hz, 1H), 4.64 (s, 2H), 4.56 (s, 2H), 3.25-3.11 (m, 1H), 2.85-2.72 (m, 2H), 2.33-2.27 (m, 2H), 2.17-2.05 (m, 2H), 1.97-1.76 (m, 6H) |
| | 2-((2-oxaspiro[3.3]heptan-6-yl)oxy)- | | | |
| | N-(2-(4,4-difluorocyclohexyl)-4-(2,5- | | | |
| | difluorophenyl)pyridin-3- | | | |
| | yl)pyrimidine-5-carboxamide | | | |
| 134 | 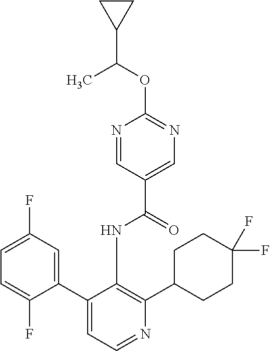 [SMILES: CC(Oc1ncc(C(=O)Nc2c(-c3cc(F)ccc3F)ccnc2C2CCC(F)(F)CC2)cn1)C1CC1] | 18% | 515.2 (M + 1) | (400 MHz, MeOD- d₄) δ 8.85 (s, 2H), 8.63 (d, J = 4.8 Hz, 1H), 7.37 (d, J = 4.8 Hz, 1H), 7.27-7.11 (m, 3H), 4.83-4.60 (m, 1H), 3.24-3.13 (m, 1H), 2.26-2.00 (m, 4H), 1.99-1.78 (m, 4H), 1.46 (d, J = 6.0 Hz, 3H), 1.27-1.15 (m, 1H), 0.67-0.52 (m, 2H), 0.52-0.44 (m, 1H), 0.40-0.33 (m. 1H) |
| | 2-(1-cyclopropylethoxy)- | | | |
| | N-(2-(4,4-difluorocyclohexyl)- | | | |
| | 4-(2,5-difluorophenyl)pyridin-3- | | | |
| | yl)pyrimidine-5-carboxamide | | | |
| 135 | 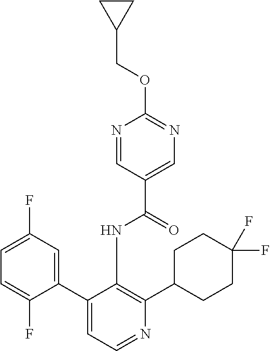 [SMILES: O=C(Nc1c(-c2cc(F)ccc2F)ccnc1C1CCC(F)(F)CC1)c1cnc(OCC2CC2)nc1] | 51% | 501.3 (M + 1) | (400 MHz, DMSO- d₆) δ 10.25 (s, 1H), 8.87 (s, 2H), 8.63 (d, J = 4.8 Hz, 1H), 7.41-7.31 (m, 2H), 7.30-7.21 (m, 2H), 4.21 (d, J = 7.2 Hz, 2H), 3.24- 3.14 (m, 1H), 2.20-2.04 (m, 2H), 2.02-1.74 (m, 6H), 1.40-1.18 (m, 1H), 0.72-0.46 (m, 2H), 0.41-0.25 (m, 2H) |
| | 2-(cyclopropylmethoxy)- | | | |
| | N-(2-(4,4-difluorocyclohexyl)-4- | | | |
| | (2,5-difluorophenyl)pyridin-3- | | | |
| | yl)pyrimidine-5-carboxamide | | | |
| 136 | 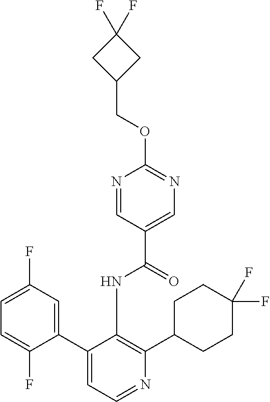 [SMILES: O=C(Nc1c(-c2cc(F)ccc2F)ccnc1C1CCC(F)(F)CC1)c1cnc(OCC2CC(F)(F)C2)nc1] | 39% | 551.3 (M + 1) | (400 MHz, CDCl₃) δ 8.83 (s, 2H), 8.69 (d, J = 4.8 Hz, 1H), 7.49 (s, 1H), 7.21 (d, J = 4.8 Hz, 1H), 7.16- 7.02 (m, 3H), 4.49 (d, J = 6.4 Hz, 2H), 3.05-2.90 (m, 1H), 2.87-2.61 (m, 3H), 2.57-2.37 (m, 2H), 2.32-2.06 (m, 4H), 1.94 (d, J = 13.2 Hz, 2H), 1.87-1.68 (m, 2H) |
| | 2-((3,3-difluorocyclobutyl) | | | |
| | methoxy)-N-(2-(4,4- | | | |
| | difluorocyclohexyl)-4-(2,5- | | | |
| | difluorophenyl)pyridin-3- | | | |
| | yl)pyrimidine-5- | | | |
| | carboxamide | | | |
| 137 | 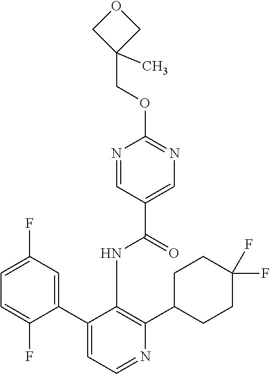 [SMILES: CC1(COc2ncc(C(=O)Nc3c(-c4cc(F)ccc4F)ccnc3C3CCC(F)(F)CC3)cn2)COC1] | 36% | 530.2 (M + 1) | (400 MHz; MeOD) δ 8.91 (s, 2H), 8.63 (d, J = 5.0 Hz, 1H), 7.38 (d, J = 5.0 Hz, 1H), 7.26-7.13 (m, 3H), 4.67 (d, J = 6.0 Hz, 2H), 4.57 (s, 2H), 4.48 (d, J = 6.0 Hz, 2H), 3.22- 3.15 (m, 1H), 2.20- 2.03 (m, 4H), 1.95- 1.83 (m, 4H), 1.46 (s, 3H) |
| | N-(2-(4,4-difluorocyclohexyl)- | | | |
| | 4-(2,5-difluorophenyl)pyridin-3-yl)- | | | |
| | 2-((3-methyloxetan-3- | | | |
| | yl)methoxy)pyrimidine-5- | | | |
| | carboxamide | | | |
| 138 | 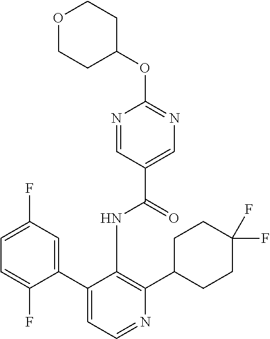 [SMILES: O=C(Nc1c(-c2cc(F)ccc2F)ccnc1C1CCC(F)(F)CC1)c1cnc(OC2CCOCC2)nc1] | 43% | 530.2 (M + 1) | (400 MHz; CDCl₃) δ 8.85 (s, 2H), 8.71 (d, J = 4.8 Hz, 1H), 7.48 (d, J = 1.5 Hz, 1H), 7.23 (d, J = 4.9 Hz, 1H), 7.17-7.07 (m, 3H), 5.31 (dt, J = 7.8, 4.7 Hz, 1H), 4.04 (dt, J = 11.3, 5.2 Hz, 2H), 3.64 (ddd, J = 11.7, 8.9, 2.9 Hz, 2H), 3.03- 2.97 (m, 1H), 2.28- 2.09 (m, 6H), 1.98- 1.72 (m, 6H) |
| | N-(2-(4,4-difluorocyclohexyl)- | | | |
| | 4-(2,5-difluorophenyl)pyridin- | | | |
| | 3-yl)-2-((tetrahydro-2H- | | | |
| | pyran-4-yl)oxy)pyrimidine- | | | |
| | 5-carboxamide | | | |
| 139 | 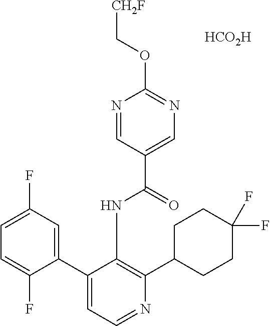 | 33% | 493.3 (M + 1) | (400 MHz, DMSO- d₆) δ 10.32 (d, J = 6.4 Hz, 1H), 8.90 (s, 2H), 8.63 (d, J = 5.2 Hz, 1H), 8.43 (d, J = 3.6 Hz, 1H), 7.45-7.32 (m, 2H), 7.31-7.20 (m, 2H), 4.89-4.79 (m, 1H), 4.78-4.70 (m, 1H), 4.68-4.63 (m, 1H), 4.63-4.55 (m, 1H), 3.25-3.15 (m, 1H), 2.18-2.04 (m, 2H), 2.02-1.79 (m, 6H) |
| | N-(2-(4,4-difluorocyclohexyl)- | | | |
| | 4-(2,5-difluorophenyl)pyridin- | | | |
| | 3-yl)-2-(2-fluoroethoxy)pyrimidine- | | | |
| | 5-carboxamide formate salt | | | |
| 140 | 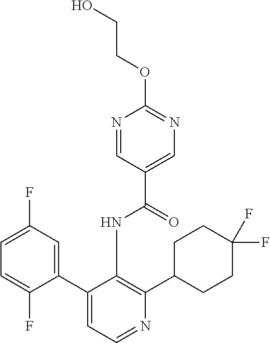 [SMILES: O=C(Nc1c(-c2cc(F)ccc2F)ccnc1C1CCC(F)(F)CC1)c1cnc(OCCO)nc1] | 25% | 491.1 (M + 1) | (400 MHz, DMSO- d₆) δ 10.22 (s, 1H), 8.87 (s, 2H), 8.62 (d, J = 4.8 Hz, 1H), 7.42-7.30 (m, 2H), 7.29-7.16 (m, 2H), 4.94 (t, J = 5.6 Hz, 1H), 4.49- 4.30 (m, 2H), 3.73 (q, J = 5.2 Hz, 2H), 3.19 (s, 1H), 2.08 (s, 2H), 2.02- 1.78 (m, 6H) |
| | N-(2-(4,4-difluorocyclohexyl)- | | | |
| | 4-(2,5-difluorophenyl) | | | |
| | pyridin-3-yl)-2-(2- | | | |
| | hydroxyethoxy)pyrimidine-5- | | | |
| | carboxamide | | | |
| 141 | 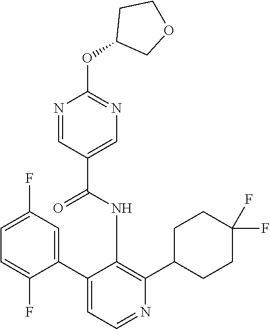 [SMILES: O=C(Nc1c(-c2cc(F)ccc2F)ccnc1C1CCC(F)(F)CC1)c1cnc(O[C@@H]2CCOC2)nc1] | 47% | 516.2 (M + 1) | ¹H-NMR (400 MHz; MeOD) δ 8.89 (s, 2H), 8.63 (d, J = 5.0 Hz, 1H), 7.37 (d, J = 5.0 Hz, 1H), 7.23-7.15 (m, 3H), 5.69-5.65 (m, 1H), 4.07-3.91 (m, 4H), 3.22-3.15 (m, 1H), 2.35 (dtd, J = 14.1, 8.2, 6.0 Hz, 1H), 2.23-2.03 (m, 5H), 1.94-1.80 (m, 4H) |
| | (R)-N-(2-(4,4- | | | |
| | difluorocyclohexyl)-4-(2,5- | | | |
| | difluorophenyl)pyridin-3-yl)-2- | | | |
| | ((tetrahydrofuran-3- | | | |
| | yl)oxy)pyrimidine- | | | |
| | 5-carboxamide | | | |
| 142 | 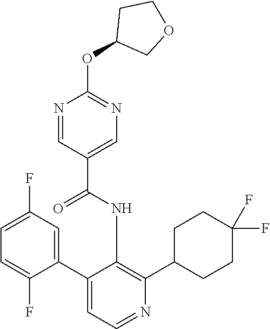 [SMILES: O=C(Nc1c(-c2cc(F)ccc2F)ccnc1C1CCC(F)(F)CC1)c1cnc(O[C@H]2CCOC2)nc1] | 46% | 516.2 (M + 1) | ¹H-NMR (400 MHz; MeOD) δ 8.89 (s, 2H), 8.63 (d, J = 5.0 Hz, 1H), 7.37 (d, J = 4.9 Hz, 1H), 7.23-7.13 (m, 3H), 5.69-5.65 (m, 1H), 4.07-3.90 (m, 4H), 3.21-3.15 (m, 1H), 2.35 (dtd, J = 14.1, 8.2, 6.0 Hz, 1H), 2.23-2.03 (m, 5H), 1.94-1.80 (m, 4H) |
| | (S)-N-(2-(4,4-difluorocyclohexyl)- | | | |
| | 4-(2,5-difluorophenyl)pyridin- | | | |
| | 3-yl)-2-((tetrahydrofuran-3- | | | |
| | yl)oxy)pyrimidine- | | | |
| | 5-carboxamide | | | |
| 143 | 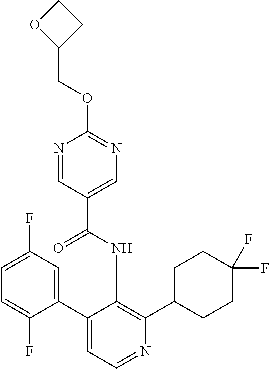 [SMILES: O=C(Nc1c(-c2cc(F)ccc2F)ccnc1C1CCC(F)(F)CC1)c1cnc(OCC2CCO2)nc1] | 26% | 516.2 (M + 1) | ¹H-NMR (400 MHz; DMSO-d₆) δ 10.25-10.24 (m, 1H), 8.89 (s, 2H), 8.63 (d, J = 4.9 Hz, 1H), 7.39-7.32 (m, 2H), 7.30-7.23 (m, 2H), 5.07-5.01 (m, 1H), 4.57-4.46 (m, 4H), 3.23-3.17 (m, 1H), 2.74-2.67 (m, 1H), 2.59-2.48, 2.13-2.01 (m, 2H), 2.01-1.80 (m, 5H) |
| | N-(2-(4,4-difluorocyclohexyl)- | | | |
| | 4-(2,5-difluorophenyl)pyridin- | | | |
| | 3-yl)-2-(oxetan-2- | | | |
| | ylmethoxy)pyrimidine-5- | | | |
| | carboxamide | | | |
| 144 | 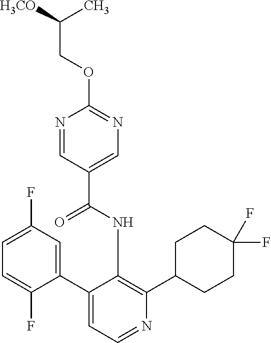 [SMILES: CO[C@@H](C)COc1ncc(C(=O)Nc2c(-c3cc(F)ccc3F)ccnc2C2CCC(F)(F)CC2)cn1] | 26% | 518.2 (M + 1) | ¹H-NMR (400 MHz; CDCl₃) δ 8.84 (s, 2H), 8.71 (d, J = 4.9 Hz, 1H), 7.51 (d, J = 1.2 Hz, 1H), 7.23 (d, J = 4.9 Hz, 1H), 7.16-7.06 (m, 3H), 4.48 (dd, J = 11.2, 6.5 Hz, 1H), 4.39 (dd, J = 11.2, 4.1 Hz, 1H), 3.81-3.77 (m, 1H), 3.45 (s, 3H), 3.05- 2.98 (m, 1H), 2.30- 2.19 (m, 2H), 2.18- 2.12 (m, 1H), 1.98- 1.94 (m, 2H), 1.89- 1.73 (m, 3H), 1.30 (d, J = 6.4 Hz, 3H) |
| | (S)-N-(2-(4,4- | | | |
| | difluorocyclohexyl)- | | | |
| | 4-(2,5-difluorophenyl)pyridin- | | | |
| | 3-yl)-2-(2-methoxypropoxy) | | | |
| | pyrimidine-5-carboxamide | | | |
| 145 | 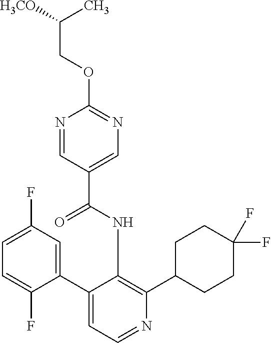 [SMILES: CO[C@H](C)COc1ncc(C(=O)Nc2c(-c3cc(F)ccc3F)ccnc2C2CCC(F)(F)CC2)cn1] | 25% | 518.2 (M + 1) | ¹H-NMR (400 MHz; MeOD) δ 8.89 (s, 2H), 8.63 (d, J = 5.0 Hz, 1H), 7.37 (d, J = 5.0 Hz, 1H), 7.24-7.18 (m, 1H), 7.17-7.13 (m, 2H), 4.45 (qd, J = 10.9, 5.1 Hz, 2H), 3.81- 3.77 (m, 1H), 3.43 (s, 3H), 3.22-3.15 (m, 1H), 2.21-2.03 (m, 4H), 1.94-1.81 (m, 4H), 1.27 (d, J = 6.4 Hz, 3H) |
| | (R)-N-(2-(4,4- | | | |
| | difluorocyclohexyl)- | | | |
| | 4-(2,5-difluorophenyl)pyridin- | | | |
| | 3-yl)-2-(2-methoxypropoxy) | | | |
| | pyrimidine-5-carboxamide | | | |
| 146 | 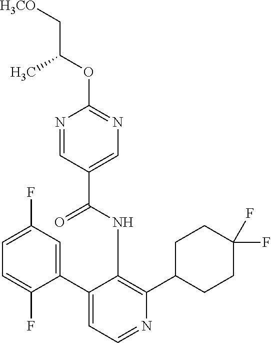 [SMILES: COC[C@@H](C)Oc1ncc(C(=O)Nc2c(-c3cc(F)ccc3F)ccnc2C2CCC(F)(F)CC2)cn1] | 14% | 518.2 (M + 1) | ¹H-NMR (400 MHz; DMSO-d₆) δ 10.23 (s, 1H), 8.87 (d, J = 5.4 Hz, 2H), 8.62 (d, J = 4.9 Hz, 1H), 7.38-7.30 (m, 2H), 7.29-7.22 (m, 2H), 5.40-5.34 (m, 1H), 3.53 (qd, J = 12.1, 5.1 Hz, 2H), 3.29 (s, 3H), 3.23-3.17 (m, 1H), 2.13-2.06 (m, 2H), 2.00-1.82 (m, 6H), 1.29 (d, J = 6.4 Hz, 3H) |
| | (R)-N-(2-(4,4- | | | |
| | difluorocyclohexyl)- | | | |
| | 4-(2,5-difluorophenyl)pyridin-3-yl)- | | | |
| | 2-((1-methoxypropan-2-yl)oxy) | | | |
| | pyrimidine-5-carboxamide | | | |
| 147 | 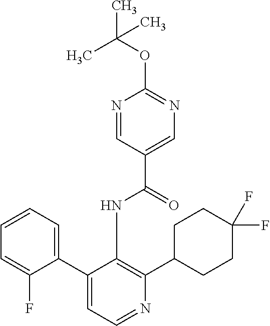 [SMILES: CC(C)(C)Oc1ncc(C(=O)Nc2c(-c3ccccc3F)ccnc2C2CCC(F)(F)CC2)cn1] | 5% | 485.3 (M + 1) | ¹H NMR (400 MHz, DMSO-d₆) δ 10.12 (s, 1H), 8.79 (s, 2H), 8.59 (d, J = 4.8 Hz, 1H), 7.44-7.38 (m, 1H), 7.37 (dd, J = 5.6, 1.6 Hz, 2H), 7.31- 7.24 (m, 1H), 7.24-7.18 (m, 1H), 3.22-3.11 (m, 1H), 2.08 (d, J = 4.0 Hz, 2H), 1.98-1.77 (m, 6H), 1.58 (s, 9H) |
| | 2-(tert-butoxy)-N-(2-(4,4- | | | |
| | difluorocyclohexyl)-4-(2- | | | |
| | fluorophenyl)pyridin-3- | | | |
| | yl)pyrimidine-5- | | | |
| | carboxamide | | | |
| 148 | 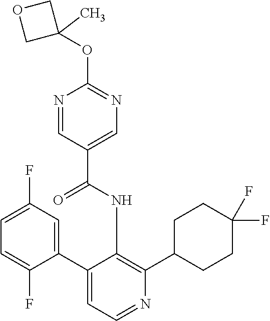 [SMILES: CC1(Oc2ncc(C(=O)Nc3c(-c4cc(F)ccc4F)ccnc3C3CCC(F)(F)CC3)cn2)COC1] | 7% | 517.3 (M + 1) | ¹H NMR (400 MHz, MeOD-d₄) δ 8.85 (s, 2H), 8.61 (d, J = 4.8 Hz, 1H), 7.35 (d, J = 4.8 Hz, 1H), 7.26- 7.10 (m, 3H), 4.90 (d, J = 7.2 Hz, 2H), 4.63 (d, J = 7.6 Hz, 2H), 3.20- 3.11 (m, 1H), 2.20-2.00 (m, 4H), 1.95-1.87 (m, 3H), 1.84 (s, 3H), 1.83- 1.76 (m, 1H) |
| | N-(2-(4,4-difluorocyclohexyl)- | | | |
| | 4-(2,5-difluorophenyl)pyridin- | | | |
| | 3-yl)-2-((3-methyloxetan-3-yl) | | | |
| | oxy)pyrimidine-5-carboxamide | | | |
| 149 | 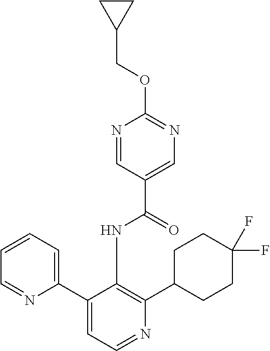 [SMILES: O=C(Nc1c(-c2ccccn2)ccnc1C1CCC(F)(F)CC1)c1cnc(OCC2CC2)nc1] | 39% | 466.2 (M + 1) | ¹H NMR (400 MHz, MeOD-d₄) δ 8.93 (s, 2H), 8.65- 8.59 (m, 2H), 8.53 (br s, 1H), 7.88 (dt, J = 7.6, 1.6 Hz, 1H), 7.64 (d, J = 8.0 Hz, 1H), 7.51 (d, J = 5.2 Hz, 1H), 7.43-7.36 (m, 1H), 4.31 (d, J = 7.2 Hz, 2H), 3.23- 3.12 (m, 1H), 2.22-2.00 (m, 4H), 1.99-1.75 (m, 4H), 1.39-1.28 (m, 1H), 0.68-0.59 (m, 2H), 0.44-0.36 (m, 2H) |
| | 2-(cyclopropylmethoxy)- | | | |
| | N-(2′-(4,4-difluorocyclohexyl)- | | | |
| | [2,4′-bipyridin]-3′-yl)pyrimidine- | | | |
| | 5-carboxamide | | | |
| 150 | 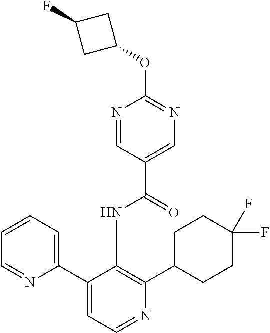 [SMILES: O=C(Nc1c(-c2ccccn2)ccnc1C1CCC(F)(F)CC1)c1cnc(O[C@H]2C[C@H](F)C2)nc1] | 42% | 483.2 (M + 1) | ¹H-NMR (400 MHz; MeOD) δ 8.96 (s, 2H), 8.65- 8.63 (m, 2H), 7.89 (td, J = 7.8, 1.7 Hz, 1H), 7.66 (d, J = 8.0 Hz, 1H), 7.52 (d, J = 5.0 Hz, 1H), 7.43-7.40 (m, 1H), 5.56-5.50 (m, 1H), 5.42-5.37 (m, 1H), 5.28-5.23 (m, 1H), 4.60 (s, 1H), 3.23-3.15 (m, 1H), 2.82-2.71 (m, 2H), 2.67-2.55 (m, 2H), 2.21-2.03 (m, 4H), 1.99-1.91 (m, 2H), 1.91-1.79 (m, 1H) |
| | N-(2′-(4,4-difluorocyclohexyl)- | | | |
| | [2,4′-bipyridin]-3′-yl)-2- | | | |
| | ((1r,3r)-3-fluorocyclobutoxy) | | | |
| | pyrimidine-5-carboxamide | | | |
| 151 | 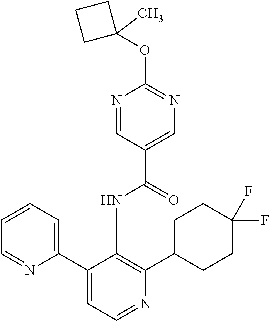 [SMILES: CC1(Oc2ncc(C(=O)Nc3c(-c4ccccn4)ccnc3C3CCC(F)(F)CC3)cn2)CCC1] | 17% | 480.1 (M + 1) | ¹H NMR (400 MHz, CDCl₃) δ 11.28 (s, 1H), 9.03 (s, 2H), 8.67 (dd, J = 4.4, 16.8 Hz, 2H), 7.93-7.84 (m, 1H), 7.74 (d, J = 8.0 Hz, 1H), 7.42- 7.31 (m, 2H), 2.98 (t, J = 10.4 Hz, 1H), 2.57-2.42 (m, 2H), 2.39-2.28 (m, 2H), 2.23 (dd, J = 6.4, 16.8 Hz, 2H), 2.17-2.00 (m, 4H), 1.92-1.81 (m, 2H), 1.80-1.73 (m, 2H), 1.72 (s, 3H) |
| | N-(2′-(4,4-difluorocyclohexyl)- | | | |
| | [2,4′-bipyridin]-3′-yl)- | | | |
| | 2-(1-methylcyclobutoxy) | | | |
| | pyrimidine-5-carboxamide | | | |
| 152 | 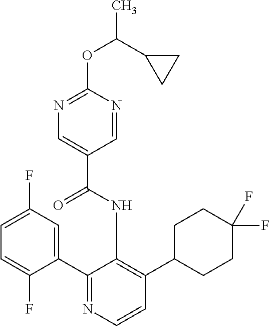 [SMILES: CC(Oc1ncc(C(=O)Nc2c(C3CCC(F)(F)CC3)ccnc2-c2cc(F)ccc2F)cn1)C1CC1] | 51% | 515.1 (M + 1) | ¹H NMR (400 MHz, DMSO-d₆) δ 10.18 (s, 1H), 8.82 (s, 2H), 8.59 (d, J = 4.4 Hz, 1H), 7.54 (d, J = 4.4 Hz, 1H), 7.34-7.16 (m, 3H), 4.77-4.61 (m, 1H), 3.05-2.97 (m, 1H), 2.17-2.04 (m, 2H), 2.03-1.80 (m, 4H), 1.78-1.63 (m, 2H), 1.36 (d, J = 6.0 Hz, 3H), 1.18 (ddt, J = 12.4, 8.8, 4.0 Hz, 1H), 0.59-0.46 (m, 2H), 0.42-0.29 (m, 2H) |
| | 2-(1-cyclopropylethoxy)-N-(4-(4,4- | | | |
| | difluorocyclohexyl)-2-(2,5- | | | |
| | difluorophenyl)pyridin-3- | | | |
| | yl)pyrimidine-5-carboxamide | | | |
| 153 | 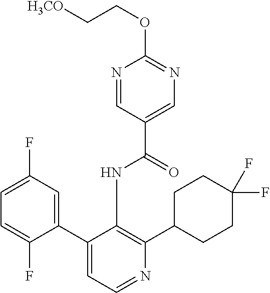 [SMILES: COCCOc1ncc(C(=O)Nc2c(-c3cc(F)ccc3F)ccnc2C2CCC(F)(F)CC2)cn1] | 48% | 505.2 (M + 1) | ¹H NMR (400 MHz, CDCl₃) δ 8.83 (s, 2H), 8.70 (d, J = 4.8 Hz, 1H), 7.50 (s, 1H), 7.21 (d, J = 4.8 Hz, 1H), 7.17-7.04 (m, 3H), 4.71-4.49 (m, 2H), 3.86-3.71 (m, 2H), 3.44 (s, 3H), 3.10-2.92 (m, 1H), 2.29-2.19 (m, 2H), 2.19-2.09 (m, 2H), 1.95 (d, J = 14.0 Hz, 2H), 1.88-1.74 (m, 2H) |
| | N-(2-(4,4-difluorocyclohexyl)- | | | |
| | 4-(2,5-difluorophenyl)pyridin- | | | |
| | 3-yl)-2-(2-methoxyethoxy) | | | |
| | pyrimidine-5-carboxamide | | | |
| 154 | 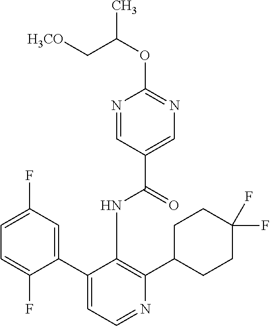 [SMILES: COCC(C)Oc1ncc(C(=O)Nc2c(-c3cc(F)ccc3F)ccnc2C2CCC(F)(F)CC2)cn1] | 35% | 519.3 (M + 1) | ¹H-NMR (400 MHz; MeOD) δ 8.87 (s, 2H), 8.63 (d, J = 5.0 Hz, 1H), 7.37 (d, J = 4.9 Hz, 1H), 7.18 (dtd, J = 17.0, 8.5, 4.4 Hz, 3H), 5.53- 5.45 (m, 1H), 3.65 (dd, J = 10.6, 6.6 Hz, 1H), 3.59 (dd, J = 10.7, 3.8 Hz, 1H), 3.39 (s, 3H), 3.21-3.15 (m, 1H), 2.20-2.02 (m, 4H), 1.94-1.80 (m, 4H), 1.38 (d, J = 6.4 Hz, 3H) |
| | N-(2-(4,4- | | | |
| | difluorocyclohexyl)-4-(2,5- | | | |
| | difluorophenyl)pyridin-3-yl)-2-((1- | | | |
| | methoxypropan-2-yl)oxy) | | | |
| | pyrimidine-5-carboxamide | | | |

### Example 155

### Synthesis of N-(2-(4,4-difluorocyclohexyl)-4-(2,5-difluorophenyl)pyridin-3-yl)-2-(2-(difluoromethoxy)ethoxy)pyrimidine-5-carboxamide

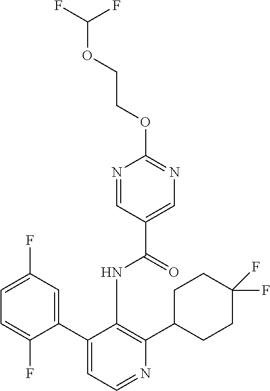 [SMILES: O=C(Nc1c(-c2cc(F)ccc2F)ccnc1C1CCC(F)(F)CC1)c1cnc(OCCOC(F)F)nc1]

To a solution of N-[2-(4,4-difluorocyclohexyl)-4-(2,5-difluorophenyl)-3-pyridyl]-2-(2-hydroxyethoxy) pyrimidine-5-carboxamide (0.0300 g, 0.061 mmol), cuprous iodide (0.00600 g, 0.0320 mmol,) in acetonitrile (2 mL) at 70° C. was added 2,2-difluoro-2-fluorosulfonyl-acetic acid (0.0330 g, 0.183 mmol) in methyl cyanide (1 mL) dropwise over 30 min. The reaction mixture was stirred at this temperature for 1 h, then 2,2-difluoro-2-fluorosulfonyl-acetic acid (0.0330 g, 0.183 mmol) was added dropwise over 30 min again. The reaction mixture was diluted with ethyl acetate (30 mL) and washed with brine (2×10 mL). The organic layer was dried over sodium sulfate and then the organic layer was concentrated under reduced pressure. The residue was purified by preparative HPLC, eluting with a gradient of 40-70% of acetonitrile in water containing 0.225% formic acid, to afford the title compound as a colorless solid (0.00800 g, 23% yield): ¹H NMR (400 MHz, DMSO-d₆) δ 10.26 (s, 1H), 8.90 (s, 2H), 8.63 (d, J=4.8 Hz, 1H), 7.32 (s, 2H), 7.31-7.16 (m, 2H), 7.01-6.50 (t, J=75.6 Hz, 1H), 4.60-4.55 (m, 2H), 4.33-4.09 (m, 2H), 3.23-3.13 (m, 1H), 2.10 (d, J=3.2 Hz, 2H), 2.01-1.76 (m, 6H); MS (ES+) m/z 541.3 (M+1).

### Example 156

### Synthesis of N-(2-(4,4-difluorocyclohexyl)-4-(2,5-difluorophenyl)pyridin-3-yl)-2-(2-(difluoromethoxy)ethoxy) pyrimidine-5-carboxamide

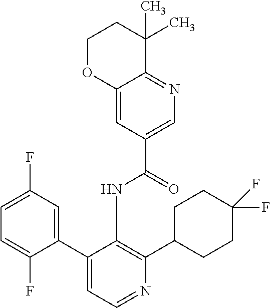 [SMILES: CC1(C)CCOc2cc(C(=O)Nc3c(-c4cc(F)ccc4F)ccnc3C3CCC(F)(F)CC3)cnc21]

### Step 1. Preparation of methyl 5-((3-methylbut-3-en-1-yl) oxy) nicotinate

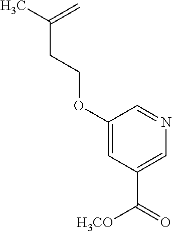 [SMILES: C=C(C)CCOc1cncc(C(=O)OC)c1]

To a mixture of methyl 5-hydroxynicotinate (4.00 g, 26.1 mmol) and 3-methylbut-3-en-1-ol (2.47 g, 28.7 mmol) in tetrahydrofuran (110 mL) was added triphenylphosphine (8.22 g, 31.3 mmol). The mixture was stirred at 25° C. under nitrogen atmosphere for 1 h. Then diethyl (E)-diazene-1,2-dicarboxylate (5.46 g, 31.3 mmol) was added at 0° C. The mixture was stirred at 25° C. for 12 h. The reaction mixture was concentrated under reduced pressure. The residue was poured into water (50 mL) and extracted with ethyl acetate (3×50 mL). The combined organic extract was dried over anhydrous sodium sulfate, filtered and the filtrate was concentrated in vacuo. The residue was purified by flash silica gel chromatography, eluting with 33% of ethyl acetate in petroleum ether, to afford the title compound as a colorless oil (4.00 g, 69% yield) as colorless oil; ¹H NMR (400 MHz, CDCl₃) δ 8.81 (d, J=1.6 Hz, 1H), 8.46 (d, J=2.8 Hz, 1H), 7.76 (dd, J=2.0, 2.8 Hz, 1H), 4.87 (s, 1H), 4.81 (s, 1H), 4.16 (t, J=6.8 Hz, 2H), 3.94 (s, 3H), 2.53 (t, J=6.8 Hz, 2H), 1.81 (s, 3H).

### Step 2. Preparation of methyl 4,4-dimethyl-3,4-dihydro-2H-pyrano[3,2-b]pyridine-7-carboxylate

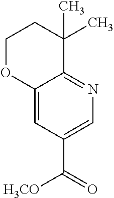 [SMILES: COC(=O)c1cnc2c(c1)OCCC2(C)C]

To a mixture of methyl 5-((3-methylbut-3-en-1-yl)oxy)nicotinate (1.00 g, 4.52 mmol) in ethanol (20 mL) was added trifluoroacetic acid (1.03 g, 9.04 mmol). The mixture was stirred at 25° C. for 0.5 h. To the mixture was added tris [(Z)-1-methyl-3-oxo-but-1-enoxy] iron (0.79 g, 2.26 mmol), phenylsilane (1.22 g, 11.3 mmol) and 2-tert-butylperoxy-2-methyl-propane (1.98 g, 13.6 mmol) under a nitrogen atmosphere. The mixture was stirred at 60° C. for 4 h. After cooling to ambient temperature, the reaction mixture was concentrated under reduced pressure. The residue was poured into water (20 mL) and extracted with ethyl acetate (3×15 mL). The combined organic extracts were dried over anhydrous sodium sulfate, filtered and concentrated in vacuo. The residue was purified by flash silica gel chromatography, eluting with a gradient of 9% of ethyl acetate in petroleum ether, to afford the title compound as a colorless oil (0.500 g, 2.26 mmol, 50% yield): ¹H NMR (400 MHz, CDCl₃) δ 8.74 (d, J=2.0 Hz, 1H), 7.65 (d, J=2.0 Hz, 1H), 4.29-4.19 (m, 2H), 3.91 (s, 3H), 1.99-1.91 (m, 2H), 1.40 (s, 6H).

### Step 3. Preparation of 4,4-dimethyl-3,4-dihydro-2H-pyrano[3,2-b]pyridine-7-carboxylic acid

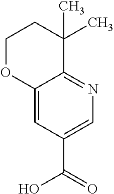

To a mixture of methyl 4,4-dimethyl-3,4-dihydro-2H-pyrano[3,2-b]pyridine-7-carboxylate (0.450 g, 2.03 mmol) in methanol (0.75 mL) and water (0.75 mL) was added sodium hydroxide (4 M, 9 mL). The mixture was stirred at 25° C. for 1 h. The reaction mixture was adjusted to pH=2 with hydrochloric acid (2 M). After filtration, the filter was concentrated in vacuo to afford the title compound as a colorless solid (0.180 g, 0.868 mmol, 43% yield): ¹H NMR (400 MHz, DMSO-d₆) δ 13.50-12.95 (m, 1H), 8.61 (d, J=2.0 Hz, 1H), 7.51 (d, J=2.0 Hz, 1H), 4.30-4.16 (m, 2H), 2.02-1.82 (m, 2H), 1.33 (s, 6H).

### Step 4. Preparation of N-(2-(4,4-difluorocyclohexyl)-4-(2,5-difluorophenyl)pyridin-3-yl)-2-(2-(difluoromethoxy)ethoxy)pyrimidine-5-carboxamide

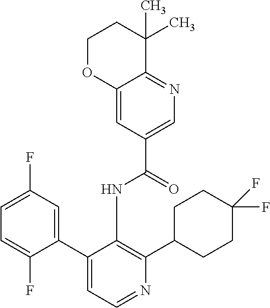 [SMILES: CC1(C)CCOc2cc(C(=O)Nc3c(-c4cc(F)ccc4F)ccnc3C3CCC(F)(F)CC3)cnc21]

To a mixture of 4,4-dimethyl-3,4-dihydro-2H-pyrano[3,2-b]pyridine-7-carboxylic acid (0.0500 g, 0.241 mmol), 2-(4,4-difluorocyclohexyl)-4-(2,5-difluorophenyl)pyridin-3-amine (0.0782 g, 0.241 mmol), 2-chloro-1-methylpyridinium iodide (0.0739 g, 0.289 mmol) in tetrahydrofuran (1.5 mL) was added N,N-diisopropylethylamine (0.0935 g, 0.723 mmol). The mixture was stirred at 65° C. for 12 h. After being cooled to room temperature, reaction mixture was concentrated under reduced pressure. The residue was poured into water (20 mL) and extracted with ethyl acetate (3×15 mL). The combined organic extract was dried over anhydrous sodium sulfate, filtered and The residue was purified by preparative HPLC, eluting with a gradient of 50-80% of acetonitrile in water containing 0.1% formic acid, to afford the title compound as a colorless solid (0.0475 g, 35% yield): ¹H NMR (400 MHz, DMSO-d₆) δ 10.16 (s, 1H), 8.60 (d, J=4.8 Hz, 1H), 8.41-8.37 (m, 1H), 7.39-7.31 (m, 3H), 7.30-7.18 (m, 2H), 4.26-4.18 (m, 2H), 3.21-3.10 (m, 1H), 2.15-2.02 (m, 2H), 2.00-1.76 (m, 8H), 1.31 (s, 6H); MS (ES+) m/z 514.4 (M+1).

### Example 157

### Synthesis of N-(2-(4,4-difluorocyclohexyl)-4-(2,5-difluorophenyl)pyridin-3-yl)-2-(3-methoxyazetidin-1-yl)pyrimidine-5-carboxamide

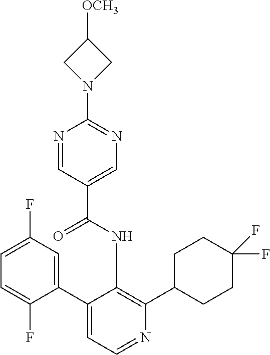 [SMILES: COC1CN(c2ncc(C(=O)Nc3c(-c4cc(F)ccc4F)ccnc3C3CCC(F)(F)CC3)cn2)C1]

To a solution of 2-chloro-N-(2-(4,4-difluorocyclohexyl)-4-(2,5-difluorophenyl)pyridin-3-yl)pyrimidine-5-carboxamide (0.0500 g, 0.108 mmol) and 3-methoxyazetidine (0.0665 g, 0.538 mmol, hydrochloride) in dimethyl sulfoxide (3 mL) was added N-ethyl-N-isopropylpropan-2-amine (0.209 g, 1.61 mmol). The mixture was stirred at 120° C. for 12 h. After being cooled to ambient temperature, ethyl acetate (10 mL) and water (10 mL) were added. The layers were separated, and the aqueous phase was extracted with ethyl acetate (3×10 mL). The combined organic extracts were washed with brine (10 mL) and then dried over anhydrous sodium sulfate and filtered. The filtrate was concentrated under reduced pressure The residue was purified by preparative HPLC, eluting with a gradient of 39-69% of acetonitrile in water containing 0.225% formic acid, to afford the title compound as a colorless solid (0.0306 g, 55% yield): ¹H NMR (400 MHz, DMSO-d₆) δ 9.91 (s, 1H), 8.64 (s, 2H), 8.59 (d, J=5.2 Hz, 1H), 7.37-7.28 (m, 2H), 7.28-7.16 (m, 2H), 4.41-4.20 (m, 3H), 3.95-3.87 (m, 2H), 3.26 (s, 3H), 3.22-3.07 (m, 1H), 2.08 (m, 2H), 2.00-1.74 (m, 6H); MS (ES+) m/z 516.4 (M+1).

### Example 158-182

In a similar manner as described in Example 157, utilizing the appropriately substituted starting materials and intermediates, the following compounds were prepared:

| Example | | | MS (ES+) | |
|---|---|---|---|---|
| No. | Structure | Yield | m/z | ¹H-NMR |
| 158 | 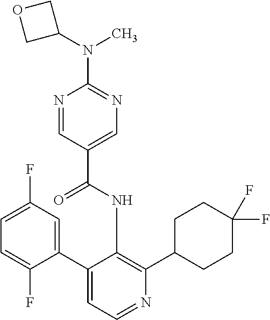 [SMILES: CN(c1ncc(C(=O)Nc2c(-c3cc(F)ccc3F)ccnc2C2CCC(F)(F)CC2)cn1)C1COC1] | 46% | 515.2 (M + 1) | ¹H-NMR (400 MHz; DMSO-d₆) δ 9.93 (s, 1H), 8.69 (s, 2H), 8.60 (d, J = 4.9 Hz, 1H), 7.35 (d, J = 4.9 Hz, 1H), 7.32 (dd, J = 9.2, 4.6 Hz, 1H), 7.28-7.19 (m, 2H), 5.48 (quintet, J = 7.3 Hz, 1H), 4.77- 4.69 (m, 4H), 3.22 (s, 3H), 3.18-3.15 (m, 1H), 2.13-2.07 (m, 2H), 1.96-1.80 (m, 6H) |
| | N-(2-(4,4- | | | |
| | difluorocyclohexyl)-4-(2,5- | | | |
| | difluorophenyl)pyridin-3-yl)- | | | |
| | 2-(methyl(oxetan-3- | | | |
| | yl)amino)pyrimidine-5- | | | |
| | carboxamide | | | |
| 159 | 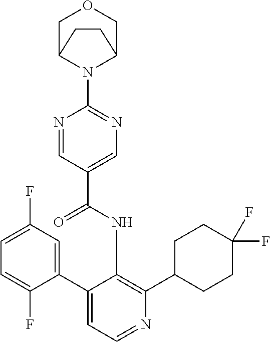 [SMILES: O=C(Nc1c(-c2cc(F)ccc2F)ccnc1C1CCC(F)(F)CC1)c1cnc(N2C3CCC2COC3)nc1] | 43% | 541.2 (M + 1) | ¹H-NMR (400 MHz; DMSO-d₆) δ 9.93 (s, 1H), 8.68 (s, 2H), 8.60 (d, J = 4.9 Hz, 1H), 7.36-7.32 (m, 2H), 7.30-7.20 (m, 2H), 4.70-7.66 (m, 2H), 3.60 (q, J = 11.3 Hz, 4H), 3.19-3.16 (m, 1H), 2.10-2.08 (m, 2H), 2.00-1.84 (m, 10H) |
| | 2-(3-oxa-8- | | | |
| | azabicyclo[3.2.1]octan-8-yl)- | | | |
| | N-(2-(4,4- | | | |
| | difluorocyclohexyl)-4-(2,5- | | | |
| | difluorophenyl)pyridin-3- | | | |
| | yl)pyrimidine-5-carboxamide | | | |
| 160 | 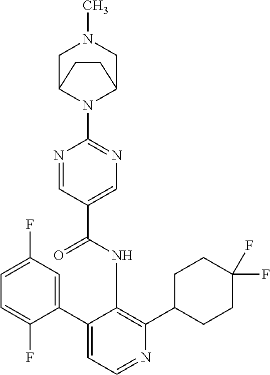 [SMILES: CN1CC2CCC(C1)N2c1ncc(C(=O)Nc2c(-c3cc(F)ccc3F)ccnc2C2CCC(F)(F)CC2)cn1] | 58% | 554.2 (M + 1) | ¹H-NMR (400 MHz; MeOD) δ 8.67 (s, 2H), 8.61 (d, J = 4.9 Hz, 1H), 7.36 (d, J = 5.0 Hz, 1H), 7.25-7.12 (m, 3H), 4.82 (d, J = 1.8 Hz, 3H), 3.21-3.14 (m, 1H), 2.82 (dd, J = 11.1, 1.9 Hz, 2H), 2.32 (d, J = 10.7 Hz, 2H), 2.26 (s, 3H), 2.20-2.11 (m, 2H), 2.09-1.79 (m, 10H) |
| | N-(2-(4,4- | | | |
| | difluorocyclohexyl)-4-(2,5- | | | |
| | difluorophenyl)pyridin-3-yl)- | | | |
| | 2-((1R,5S)-3-methyl-3,8- | | | |
| | diazabicyclo[3.2.1]octan-8- | | | |
| | yl)pyrimidine-5-carboxamide | | | |
| 161 | 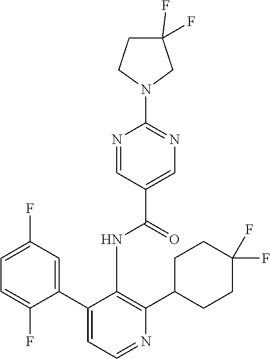 [SMILES: O=C(Nc1c(-c2cc(F)ccc2F)ccnc1C1CCC(F)(F)CC1)c1cnc(N2CCC(F)(F)C2)nc1] | 66% | 536.3 (M + 1) | ¹H NMR (400 MHz, DMSO-d₆) δ 9.99 (s, 1H), 8.72 (s, 2H), 8.60 (d, J = 4.8 Hz, 1H), 7.38-7.29 (m, 2H), 7.28-7.18 (m, 2H), 3.96 (t, J = 13.2 Hz, 2H), 3.77 (t, J = 7.2 Hz, 2H), 3.18 (dd, J = 8.4, 4.0 Hz, 1H), 2.61-2.55 (m, 2H), 2.09 (s, 2H), 1.99- 1.78 (m, 6H) |
| | N-(2-(4,4- | | | |
| | difluorocyclohexyl)-4-(2, 5- | | | |
| | difluorophenyl)pyridin-3-yl)- | | | |
| | 2-(3,3-difluoropyrrolidin-1- | | | |
| | yl)pyrimidine-5-carboxamide | | | |
| 162 | 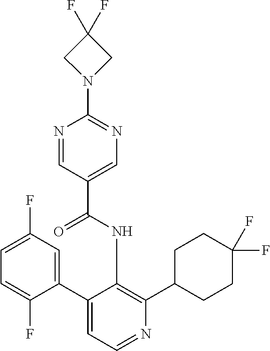 [SMILES: O=C(Nc1c(-c2cc(F)ccc2F)ccnc1C1CCC(F)(F)CC1)c1cnc(N2CC(F)(F)C2)nc1] | 65% | 521.2 (M + 1) | ¹H-NMR (400 MHz; DMSO-d₆) δ 10.06 (s, 1H), 8.74 (s, 2H), 8.61 (d, J = 4.9 Hz, 1H), 7.36 (d, J = 3.5 Hz, 1H), 7.32 (m, 1H), 7.29-7.20 (m, 2H), 4.56 (t, J = 12.4 Hz, 4H), 3.20-3.15 (m, 1H), 2.11-2.09 (m, 2H), 1.95-1.81 (m, 6H) |
| | 2-(3,3-difluoroazetidin-1-yl)- | | | |
| | N-(2-(4,4- | | | |
| | difluorocyclohexyl)-4-(2,5- | | | |
| | difluorophenyl)pyridin-3- | | | |
| | yl)pyrimidine-5-carboxamide | | | |
| 163 | 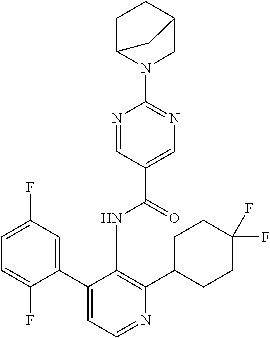 [SMILES: O=C(Nc1c(-c2cc(F)ccc2F)ccnc1C1CCC(F)(F)CC1)c1cnc(N2CC3CCC2C3)nc1] | 47% | 525.2 (M + 1) | ¹H-NMR (400 MHz; DMSO-d₆) δ 9.84 (s, 1H), 8.62 (s, 2H), 8.59 (d, J = 4.9 Hz, 1H), 7.37-7.31 (m, 2H), 7.29-7.19 (m, 2H), 4.68 (s, 1H), 3.43-3.40 (m, 1H), 3.17 (d, J = 10.0 Hz, 2H), 2.68- 2.65 (m, 1H), 2.14- 2.06 (m, 2H), 1.98- 1.77 (m, 6H), 1.73- 1.67 (m, 3H), 1.55- 1.50 (m, 2H), 1.42- 1.37 (m, 1H) |
| | 2-(2- | | | |
| | azabicyclo[2.2.1]heptan-2- | | | |
| | yl)-N-(2-(4,4- | | | |
| | difluorocyclohexyl)-4-(2,5- | | | |
| | difluorophenyl)pyridin-3- | | | |
| | yl)pyrimidine-5-carboxamide | | | |
| 164 | 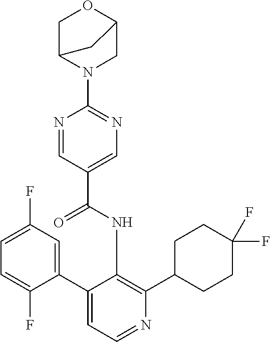 [SMILES: O=C(Nc1c(-c2cc(F)ccc2F)ccnc1C1CCC(F)(F)CC1)c1cnc(N2CC3CC2CO3)nc1] | 57% | 527.2 (M + 1) | ¹H-NMR (400 MHz; DMSO-d₆) δ 9.93 (s, 1H), 8.67 (s, 2H), 8.60 (d, J = 4.9 Hz, 1H), 7.35 (d, J = 3.6 Hz, 1H), 7.34-7.30 (m, 1H), 7.29-7.20 (m, 2H), 5.02 (s, 1H), 4.70 (d, J = 0.6 Hz, 1H), 3.81 (d, J = 0.6 Hz, 1H), 3.66 (d, J = 7.4 Hz, 1H), 3.51 (d, J = 10.2 Hz, 3H), 3.20- 3.15 (m, 1H), 2.11- 2.08 (m, 2H), 1.95- 1.81 (m, 7H) |
| | 2-(2-oxa-5- | | | |
| | azabicyclo[2.2.1]heptan-5- | | | |
| | yl)-N-(2-(4,4- | | | |
| | difluorocyclohexyl)-4-(2,5- | | | |
| | difluorophenyl)pyridin-3- | | | |
| | yl)pyrimidine-5-carboxamide | | | |
| 165 | 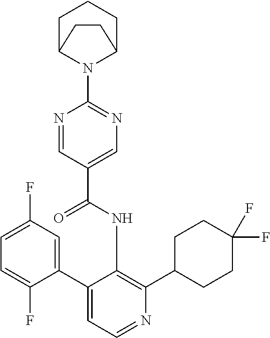 [SMILES: O=C(Nc1c(-c2cc(F)ccc2F)ccnc1C1CCC(F)(F)CC1)c1cnc(N2C3CCCC2CC3)nc1] | 51% | 539.2 (M + 1) | ¹H-NMR (400 MHz; MeOD) δ 8.65 (s, 2H), 8.60 (d, J = 5.0 Hz, 1H), 7.36 (d, J = 5.0 Hz, 1H), 7.25-7.12 (m, 3H), 4.78 (m, 2H), 4.60 (s, 1H), 3.21-3.15 (m, 1H), 2.20-2.13 (m, 2H), 2.09-1.99 (m, 4H), 1.99-1.89 (m, 5H), 1.84-1.75 (m, 3H), 1.63-1.55 (m, 3H) |
| | 2-(8-azabicyclo[3.2.1]octan- | | | |
| | 8-yl)-N-(2-(4,4- | | | |
| | difluorocyclohexyl)-4-(2,5- | | | |
| | difluorophenyl)pyridin-3- | | | |
| | yl)pyrimidine-5-carboxamide | | | |
| 166 | 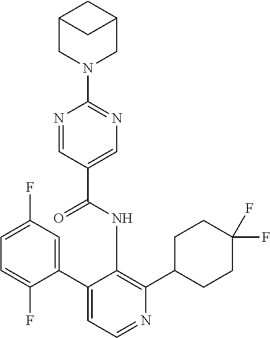 [SMILES: O=C(Nc1c(-c2cc(F)ccc2F)ccnc1C1CCC(F)(F)CC1)c1cnc(N2CC3CC(C3)C2)nc1] | 46% | 525.2 (M + 1) | ¹H-NMR (400 MHz; DMSO-d₆) δ 9.91 (s, 1H), 8.73 (s, 2H), 8.60 (d, J = 4.9 Hz, 1H), 7.36 (d, J = 4.7 Hz, 1H), 7.33 (dd, J = 9.2, 4.6 Hz, 1H), 7.24 (dtt, J = 15.0, 6.4, 3.3 Hz, 2H), 3.76 (m, 4H), 3.19-3.17 (m, 1H), 2.56 (t, J = 5.9 Hz, 2H), 2.21-2.16 (m, 2H), 2.11-2.07 (m, 2H), 1.94-1.85 (m, 6H), 1.40-1.35 (m, 2H) |
| | 2-(3- | | | |
| | azabicyclo[3.1.1]heptan-3- | | | |
| | yl)-N-(2-(4,4- | | | |
| | difluorocyclohexyl)-4-(2,5- | | | |
| | difluorophenyl)pyridin-3- | | | |
| | yl)pyrimidine-5-carboxamide | | | |
| 167 | 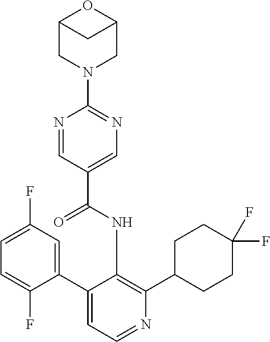 [SMILES: O=C(Nc1c(-c2cc(F)ccc2F)ccnc1C1CCC(F)(F)CC1)c1cnc(N2CC3CC(C2)O3)nc1] | 38% | 527.2 (M + 1) | ¹H-NMR (400 MHz; DMSO-d₆) δ 9.97 (s, 1H), 8.75 (s, 2H), 8.61 (d, J = 4.9 Hz, 1H), 7.36 (d, J = 4.8 Hz, 1H), 7.33 (dd, J = 9.2, 4.6 Hz, 1H), 7.29-7.20 (m, 2H), 4.71 (d, J = 6.5 Hz, 2H), 3.89 (d, J = 13.3 Hz, 2H), 3.69 (dd, J = 13.1, 0.5 Hz, 2H), 3.21-3.12 (m, 3H), 2.13-2.07 (m, 2H), 1.96-1.81 (m, 6H) |
| | 2-(6-oxa-3- | | | |
| | azabicyclo[3.1.1]heptan-3- | | | |
| | yl)-N-(2-(4,4- | | | |
| | difluorocyclohexyl)-4-(2,5- | | | |
| | difluorophenyl)pyridin-3- | | | |
| | yl)pyrimidine-5-carboxamide | | | |
| 168 | 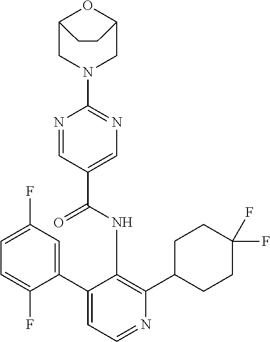 [SMILES: O=C(Nc1c(-c2cc(F)ccc2F)ccnc1C1CCC(F)(F)CC1)c1cnc(N2CC3CCC(C2)O3)nc1] | 41% | 541.2 (M + 1) | ¹H-NMR (400 MHz; MeOD) δ 8.67 (s, 2H), 8.61 (d, J = 5.0 Hz, 1H), 7.36 (d, J = 5.0 Hz, 1H), 7.24-7.11 (m, 3H), 4.63 (s, 1H), 4.47-4.46 (m, 1H), 4.46-4.42 (m, 2H), 3.22 (dd, J = 13.4, 2.1 Hz, 2H), 3.19-3.14 (m, 1H), 2.19-2.12 (m, 2H), 2.10-2.02 (m, 2H), 1.97-1.86 (m, 5H), 1.83-1.81 (m, 1H), 1.78-1.73 (m, 2H) |
| | 2-(8-oxa-3- | | | |
| | azabicyclo[3.2.1]octan-3-yl)- | | | |
| | N-(2-(4,4- | | | |
| | difluorocyclohexyl)-4-(2,5- | | | |
| | difluorophenyl)pyridin-3- | | | |
| | yl)pyrimidine-5-carboxamide | | | |
| 169 | 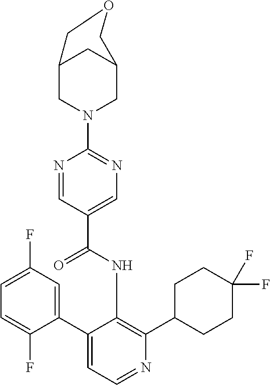 [SMILES: O=C(Nc1c(-c2cc(F)ccc2F)ccnc1C1CCC(F)(F)CC1)c1cnc(N2CC3COCC(C3)C2)nc1] | 47% | 555.2 (M + 1) | ¹H-NMR (400 MHz; DMSO-d₆) δ 9.84 (s, 1H), 8.63 (s, 2H), 8.60 (d, J = 4.9 Hz, 1H), 7.36-7.30 (m, 2H), 7.28-7.19 (m, 2H), 4.85 (d, J = 13.4 Hz, 2H), 3.79 (d, J = 11.2 Hz, 2H), 3.62 (d, J = 11.2 Hz, 2H), 3.23- 3.20 (m, 3H), 2.13- 2.06 (m, 2H), 1.96- 1.83 (m, 10H) |
| | 2-(3-oxa-7- | | | |
| | azabicyclo[3.3.1]nonan-7- | | | |
| | yl)-N-(2-(4,4- | | | |
| | difluorocyclohexyl)-4-(2,5- | | | |
| | difluorophenyl)pyridin-3- | | | |
| | yl)pyrimidine-5-carboxamide | | | |
| 170 | 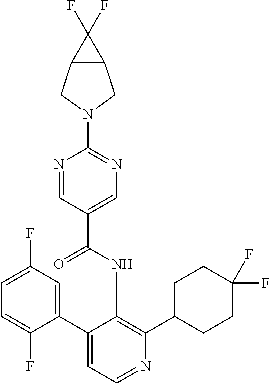 [SMILES: O=C(Nc1c(-c2cc(F)ccc2F)ccnc1C1CCC(F)(F)CC1)c1cnc(N2CC3C(C2)C3(F)F)nc1] | 37% | 547.2 (M + 1) | ¹H-NMR (400 MHz; DMSO-d₆) δ 9.92 (s, 1H), 8.68 (s, 2H), 8.60 (d, J = 4.9 Hz, 1H), 7.36 (d, J = 4.7 Hz, 1H), 7.32 (dd, J = 9.1, 4.6 Hz, 1H), 7.28-7.19 (m, 2H), 3.96 (d, J = 12.2 Hz, 2H), 3.84- 3.80 (m, 2H), 3.21- 3.13 (m, 1H), 2.72- 2.68 (m, 2H), 2.12- 2.06 (m, 2H), 1.96- 1.80 (m, 6H) |
| | 2-(6,6-difluoro-3- | | | |
| | azabicyclo[3.1.0]hexan-3- | | | |
| | yl)-N-(2-(4,4- | | | |
| | difluorocyclohexyl)-4-(2,5- | | | |
| | difluorophenyl)pyridin-3- | | | |
| | yl)pyrimidine-5-carboxamide | | | |
| 171 | 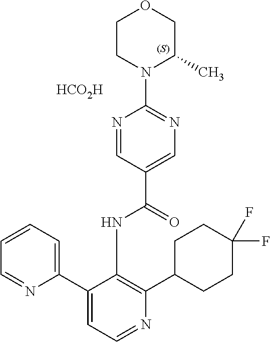 | 27% | 495.2 (M + 1) | ¹H NMR (400 MHz, DMSO-d₆) δ 10.04 (s, 1H), 8.78 (s, 2H), 8.65 (d, J = 4.8 Hz, 1H), 8.61 (d, J = 4.8 Hz, 1H), 8.47-8.41 (m, 1H), 7.84 (t, J = 7.6 Hz, 1H), 7.61 (d, J = 7.8 Hz, 1H), 7.50 (d, J = 4.8 Hz, 1H), 7.37 (dd, J = 7.4, 5.0 Hz, 1H), 4.80-4.61 (m, 1H), 4.36 (d, J = 13.0 Hz, 1H), 3.93 (dd, J = 11.2, 3.2 Hz, 1H), 3.73 (d, J = 11.4 Hz, 1H), 3.57 (dd, J = 11.4, 3.0 Hz, 1H), 3.46-3.41 (m, 1H), 3.26-3.19 (m, 1H), 3.14 (d, J = 6.6 Hz, 1H), 2.15-2.04 (m, |
| | (S)-N-(2′-(4,4- | | | 2H), 1.97-1.81 (m, |
| | difluorocyclohexyl)-[2,4′- | | | 6H), 1.22 (d, J = 6.8 |
| | bipyridin]-3′-yl)-2-(3- | | | Hz, 3H) |
| | methylmorpholino)pyrimidine- | | | |
| | 5-carboxamide formate salt | | | |
| 172 | 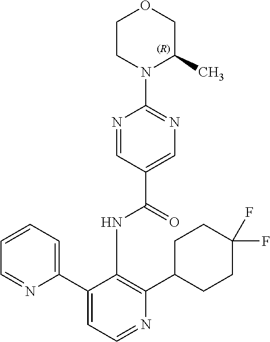 | 53% | 495.2 (M + 1) | ¹H-NMR (400 MHz; DMSO-d₆) δ 10.05 (s, 1H), 8.78 (s, 2H), 8.66-8.61 (m, 2H), 7.87-7.82 (m, 1H), 7.63-7.61 (m, 1H), 7.51 (d, J = 4.9 Hz, 1H), 7.40-7.37 (m, 1H), 4.73-4.69 (m, 1H), 4.38-4.34 (m, 1H), 3.96-3.92 (m, 1H), 3.75-3.72 (m, 1H), 3.60-3.56 (m, 1H), 3.47-3.38 (m, 2H), 3.29-3.13 (m, 2H), 2.12-2.06 (m, 2H), 1.96-1.83 (m, 6H), 1.22 (d, J = 6.8 Hz, 3H). |
| | (R)-N-(2′-(4,4- | | | |
| | difluorocyclohexyl)-[2,4′- | | | |
| | bipyridin]-3′-yl)-2-(3- | | | |
| | methylmorpholino)pyrimidine- | | | |
| | 5-carboxamide | | | |
| 173 | 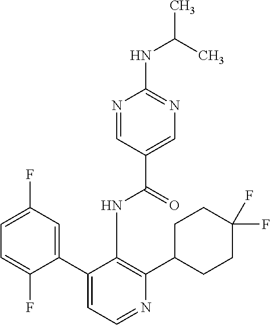 [SMILES: CC(C)Nc1ncc(C(=O)Nc2c(-c3cc(F)ccc3F)ccnc2C2CCC(F)(F)CC2)cn1] | 49% | 488.2 (M + 1) | ¹H NMR (400 MHz, CDCl₃) δ 8.66 (d, J = 4.8 Hz, 1H), 8.62 (s, 2H), 7.19 (d, J = 4.8 Hz, 1H), 7.17-7.02 (m, 3H), 6.99-6.96 (m, 1H), 5.43 (d, J = 7.6 Hz, 1H), 4.30- 4.12 (m, 1H), 3.10- 2.91 (m, 1H), 2.29- 2.19 (m, 2H), 2.18- 2.03 (m, 2H), 1.95 (d, J = 13.6 Hz, 2H), 1.89-1.71 (m, 2H), 1.27 (d, J = 6.4 Hz, 6H) |
| | N-(2-(4,4- | | | |
| | difluorocyclohexyl)-4-(2,5- | | | |
| | difluorophenyl)pyridin-3-yl)-2- | | | |
| | (isopropylamino)pyrimidine- | | | |
| | 5-carboxamide | | | |
| 174 | 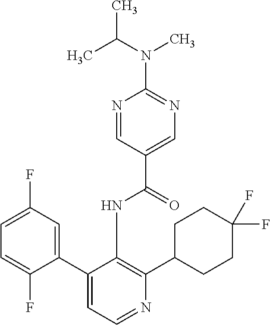 [SMILES: CC(C)N(C)c1ncc(C(=O)Nc2c(-c3cc(F)ccc3F)ccnc2C2CCC(F)(F)CC2)cn1] | 36% | 502.1 (M + 1) | ¹H NMR (400 MHz, DMSO-d₆) δ 9.87 (s, 1H), 8.65 (s, 2H), 8.59 (d, J = 4.8 Hz, 1H), 7.36-7.30 (m, 2H), 7.25 (dd, J = 7.6, 3.6 Hz, 1H), 7.23-7.18 (m, 1H), 5.04 (m, 1H), 3.18-3.12 (m, 1H), 2.97 (s, 3H), 2.14- 2.04 (m, 2H), 1.96- 1.89 (m, 2H), 1.84 (s, 4H), 1.14 (d, J = 6.8 Hz, 6H) |
| | N-(2-(4,4- | | | |
| | difluorocyclohexyl)-4-(2,5- | | | |
| | difluorophenyl)pyridin-3-yl)-2- | | | |
| | (isopropyl(methyl)amino) | | | |
| | pyrimidine-5-carboxamide | | | |
| 175 | 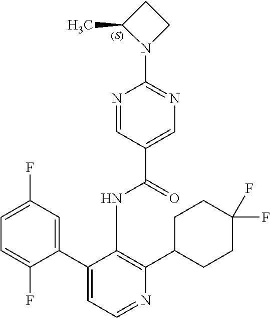 | 37% | 500.3 (M + 1) | ¹H-NMR (400 MHz; CDCl₃) δ 8.67 (d, J = 4.9 Hz, 1H), 8.63 (s, 1H), 7.30-7.26 (m, 3H), 7.20 (d, J = 4.9 Hz, 1H), 7.14-7.06 (m, 2H), 4.64-4.58 (m, 1H), 4.22-4.16 (m, 1H), 4.14-4.07 (m, 1H), 3.04-2.96 (m, 1H), 2.62-2.53 (m, 1H), 2.30-2.18 (m, 2H), 2.16-2.11 (m, 1H), 2.10-2.01 (m, 1H), 1.98-1.92 (m, 1H), 1.88-1.71 (m, 2H), 1.57 (d, J = 6.28 Hz, 3H) |
| | (S)-N-(2-(4,4- | | | |
| | difluorocyclohexyl)-4-(2,5- | | | |
| | difluorophenyl)pyridin-3-yl)- | | | |
| | 2-(2-methylazetidin-1- | | | |
| | yl)pyrimidine-5-carboxamide | | | |
| 176 | 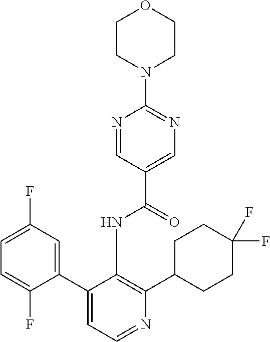 [SMILES: O=C(Nc1c(-c2cc(F)ccc2F)ccnc1C1CCC(F)(F)CC1)c1cnc(N2CCOCC2)nc1] | 48% | 516.3 (M + 1) | ¹H-NMR (400 MHz; CDCl₃) δ 8.69-8.66 (m, 3H), 7.30-7.28 (m, 2H), 7.21 (d, J = 4.9 Hz, 1H), 7.17-7.06 (m, 2H), 3.93 (t, J = 4.8 Hz, 4H), 3.79 (t, J = 4.8 Hz, 4H), 3.04-2.97 (m, 1H), 2.29-2.20 (m, 2H), 2.20-2.09 (m, 2H), 1.98-1.94 (m, 2H), 1.87-1.71 (m, 2H). |
| | N-(2-(4,4- | | | |
| | difluorocyclohexyl)-4-(2,5- | | | |
| | difluorophenyl)pyridin-3-yl)- | | | |
| | 2-morpholinopyrimidine-5- | | | |
| | carboxamide | | | |
| 177 | 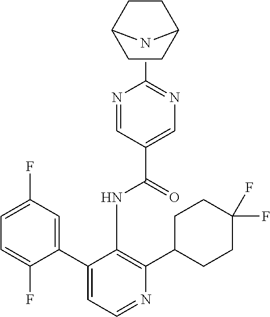 | 38% | 526.2 (M + 1) | ¹H-NMR (400 MHz; CDCl₃) δ 8.68 (d, J = 4.9 Hz, 1H), 8.63 (s, 1H), 7.28 (s, 2H), 7.21-7.16 (m, 1H), 7.15-7.06 (m, 2H), 4.85-4.77 (m, 1H), 3.05-2.97 (m, 1H), 2.28-2.22 (m, 1H), 2.17-2.10 (m, 1H), 1.99-1.94 (m, 1H), 1.86-1.84 (m, 3H), 1.80-1.71 (m, 2H), 1.60-1.54 (m, 9H). |
| | 2-(7- | | | |
| | azabicyclo[2.2.1]heptan-7- | | | |
| | yl)-N-(2-(4,4- | | | |
| | difluorocyclohexyl)-4-(2,5- | | | |
| | difluorophenyl)pyridin-3- | | | |
| | yl)pyrimidine-5-carboxamide | | | |
| 178 | 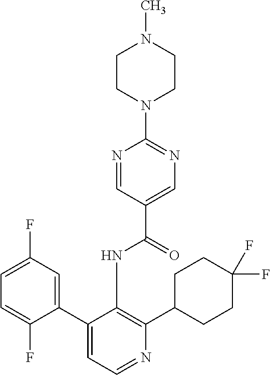 [SMILES: CN1CCN(c2ncc(C(=O)Nc3c(-c4cc(F)ccc4F)ccnc3C3CCC(F)(F)CC3)cn2)CC1] | 52% | 529.3 (M + 1) | ¹H-NMR (400 MHz; CDCl₃) δ 8.68-8.65 (m, 3H), 7.33 (d, J = 1.4 Hz, 1H), 7.21 (d, J = 4.9 Hz, 1H), 7.14- 7.06 (m, 3H), 4.00- 3.98 (m, 4H), 3.05- 2.97 (m, 1H), 2.56 (t, J = 4.9 Hz, 4H), 2.40 (s, 3H), 2.28-2.20 (m, 2H), 2.16-2.09 (m, 2H), 2.03-1.93 (m, 2H), 1.87-1.67 (m, 2H) |
| | N-(2-(4,4- | | | |
| | difluorocyclohexyl)-4-(2,5- | | | |
| | difluorophenyl)pyridin-3-yl)- | | | |
| | 2-(4-methylpiperazin-1- | | | |
| | yl)pyrimidine-5-carboxamide | | | |
| 179 | 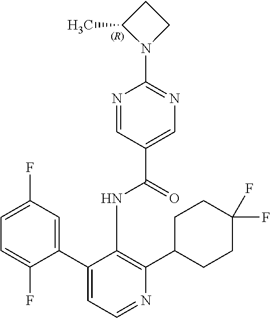 | 13% | 500.3 (M + 1) | ¹H NMR (400 MHz, CDCl₃) δ 8.65 (d, J = 4.8 Hz, 1H), 8.61 (s, 2H), 7.18 (d, J = 4.8 Hz, 1H), 7.15-7.10 (m, 1H), 7.09-7.02 (m, 2H), 4.65-4.55 (m, 1H), 4.22-4.14 (m, 1H), 4.13-4.03 (m, 1H), 2.99 (t, J = 10.8 Hz, 1H), 2.62- 2.49 (m, 1H), 2.23 (s, 2H), 2.18-2.07 (m, 2H), 2.06-2.00 (m, 1H), 2.00-1.90 (m, 2H), 1.84 (d, J = 13.6 Hz, 1H), 1.72 (d, J = 13.2 Hz, 1H), 1.56 (d, J = 6.0 Hz, 3H) |
| | (R)-N-(2-(4,4- | | | |
| | difluorocyclohexyl)-4-(2,5- | | | |
| | difluorophenyl)pyridin-3-yl)- | | | |
| | 2-(2-methylazetidin-1- | | | |
| | yl)pyrimidine-5-carboxamide | | | |
| 180 | 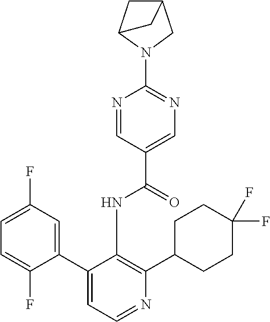 [SMILES: O=C(Nc1c(-c2cc(F)ccc2F)ccnc1C1CCC(F)(F)CC1)c1cnc(N2CC3CC2C3)nc1] | 60% | 512.3 (M + 1) | ¹H NMR (400 MHz, CDCl₃) δ 8.67 (d, J = 4.8 Hz, 1H), 8.63 (d, J = 2.8 Hz, 2H), 7.33- 7.29 (m, 1H), 7.20 (d, J = 4.8 Hz, 1H), 7.17- 7.03 (m, 3H), 5.02 (d, J = 7.0 Hz, 1H), 3.59 (s, 2H), 3.12-2.94 (m, 2H), 2.30-2.18 (m, 2H), 2.17-2.05 (m, 4H), 1.98-1.95 (m, 2H), 1.87-1.71 (m, 2H), 1.52 (dd, J = 4.8, 1.8 Hz, 2H) |
| | 2-(2- | | | |
| | azabicyclo[2.1.1]hexan-2- | | | |
| | yl)-N-(2-(4,4- | | | |
| | difluorocyclohexyl)-4-(2,5- | | | |
| | difluorophenyl)pyridin-3- | | | |
| | yl)pyrimidine-5-carboxamide | | | |
| 181 | 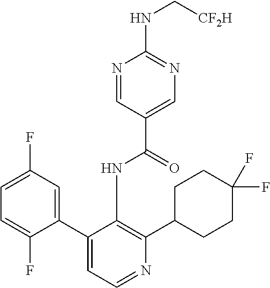 [SMILES: O=C(Nc1c(-c2cc(F)ccc2F)ccnc1C1CCC(F)(F)CC1)c1cnc(NCC(F)F)nc1] | 42% | 510.0 (M + 1) | ¹H NMR (400 MHz, DMSO-d₆) δ 9.93 (s, 1H), 8.70-8.62 (m, 2H), 8.59 (d, J = 4.8 Hz, 1H), 8.20 (t, J = 6.0 Hz, 1H), 7.36- 7.30 (m, 2H), 7.28- 7.24 (m, 1H), 7.23- 7.18 (m, 1H), 6.28- 5.93 (m, 1H), 3.83- 3.68 (m, 2H), 3.19- 3.13 (m, 1H), 2.13- 2.05 (m, 2H), 1.99- 1.91 (m, 2H), 1.89- 1.79 (m, 4H) |
| | N-(2-(4,4- | | | |
| | difluorocyclohexyl)-4-(2,5- | | | |
| | difluorophenyl)pyridin-3-yl)- | | | |
| | 2-((2,2-difluoroethyl)amino) | | | |
| | pyrimidine-5-carboxamide | | | |
| 182 | 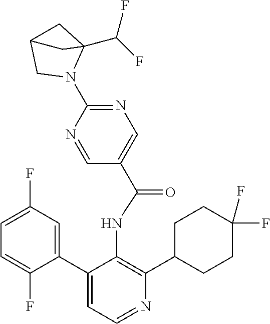 [SMILES: O=C(Nc1c(-c2cc(F)ccc2F)ccnc1C1CCC(F)(F)CC1)c1cnc(N2CC3CC2(C(F)F)C3)nc1] | 19% | 562.3 (M + 1) | ¹H NMR (400 MHz, DMSO-d₆) δ 9.94 (s, 1H), 8.68 (s, 2H), 8.59 (d, J = 4.9 Hz, 1H), 7.45-7.17 (m, 5H), 3.65 (s,2H), 3.18-3.14 (m, 1H), 2.93-2.92 (m, 1H), 2.16-1.80 (m, 10H), 1.67-1.61 (m, 2H) |
| | N-(2-(4,4- | | | |
| | difluorocyclohexyl)-4-(2,5- | | | |
| | difluorophenyl)pyridin-3-yl)- | | | |
| | 2-(1-(difluoromethyl)-2- | | | |
| | azabicyclo[2.1.1]hexan-2- | | | |
| | yl)pyrimidine-5-carboxamide | | | |

### Example 183

### Synthesis of N-(2-(4,4-difluorocyclohexyl)-4-(2,5-difluorophenyl)pyridin-3-yl)-2-(morpholine-4-carbonyl)pyrimidine-5-carboxamide

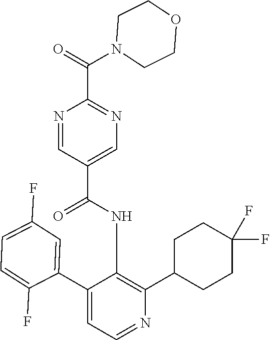 [SMILES: O=C(Nc1c(-c2cc(F)ccc2F)ccnc1C1CCC(F)(F)CC1)c1cnc(C(=O)N2CCOCC2)nc1]

### Step 1. Preparation of (5-bromopyrimidin-2-yl)(morpholino)methanone

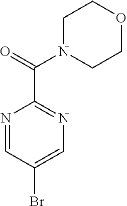 [SMILES: O=C(c1ncc(Br)cn1)N1CCOCC1]

To a mixture of 5-bromopyrimidine-2-carboxylic acid (1.00 g, 4.93 mmol), diisopropylethylamine (1.91 g, 14.8 mmol) and 2-(3H-[1,2,3]triazolo[4,5-b]pyridin-3-yl)-1,1,3,3-tetramethylisouronium hexafluoro phosphate(V) (2.25 g, 5.91 mmol) in dimethyl formamide (10 mL) was added morpholine (0.644 g, 7.39 mmol) in one portion at 20° C. The mixture was stirred at 20° C. for 12 h. The mixture was poured into water (10 mL) and extracted with ethyl acetate (3×10 mL). The combined organic layer was washed with brine (10 mL), dried over anhydrous sodium sulfate, filtered and the filtrate was evaporated under reduced pressure. The residue was purified by reversed phase chromatography, eluting with 0.1% of ammonium hydroxide in water, to afford the title compound as a yellow solid (1.00 g, 74% yield): ¹H NMR (400 MHz, DMSO-d₆) 59.10 (s, 2H), 3.71-3.60 (m, 4H), 3.54-3.48 (m, 2H), 3.24-3.19 (m, 2H).

### Step 2. Preparation of methyl 2-(morpholine-4-carbonyl)pyrimidine-5-carboxylate

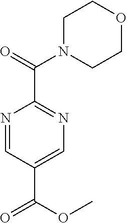 [SMILES: COC(=O)c1cnc(C(=O)N2CCOCC2)nc1]

To a mixture of (5-bromopyrimidin-2-yl)-morpholino-methanone (0.900 g, 3.31 mmol) and triethylamine (1.00 g, 9.92 mmol) in methanol (90 mL) was added [1,1′-bis(diphenylphosphino)ferrocene]dichloro-palladium(II) (0.242 g, 0.331 mmol) in one portion at 20° C. The mixture was stirred at 80° C. under carbon monoxide (50 psi) atmosphere for 16 h. The mixture was cooled to 20° C. and evaporated under reduced pressure. The residue was purified by flash silica gel chromatography, eluting with a gradient of 95-100% of ethyl acetate in petroleum ether, to afford the title compound as a brown solid (0.700 g, 84% yield): ¹H NMR (400 MHz, DMSO-d₆) δ 9.31 (s, 2H), 3.93 (s, 3H), 3.67 (s, 4H), 3.56-3.48 (m, 2H), 3.22-3.15 (m, 2H).

### Step 3. Preparation of 2-(morpholine-4-carbonyl)pyrimidine-5-carboxylic acid

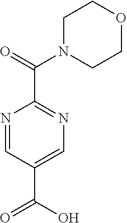 [SMILES: O=C(O)c1cnc(C(=O)N2CCOCC2)nc1]

To a solution of methyl 2-(morpholine-4-carbonyl)pyrimidine-5-carboxylate (0.200 g, 0.796 mmol) in tetrahydrofuran (5 mL) and water (5 mL) was added potassium carbonate (0.165 g, 1.19 mmol) in one portion at 20° C. The mixture was stirred at 20° C. for 12 h. The mixture was adjusted pH=3-4 with 10 wt % aqueous hydrochloric acid. The mixture was extracted with ethyl acetate (20 mL×5). The combined organic layers were washed with brine (10 mL), dried over anhydrous sodium sulfate, filtered and the filtrate was evaporated under reduced pressure to afford the title compound as a brown solid (0.0500 g, 26% yield): ¹H NMR (400 MHz, DMSO-d₆) δ 9.28 (s, 2H), 3.67 (s, 4H), 3.52-3.50 (m, 2H), 3.20-3.17 (m, 2H).

### Step 4. Preparation of N-(2-(4,4-difluorocyclohexyl)-4-(2,5-difluorophenyl)pyridin-3-yl)-2-(morpholine-4-carbonyl)pyrimidine-5-carboxamide

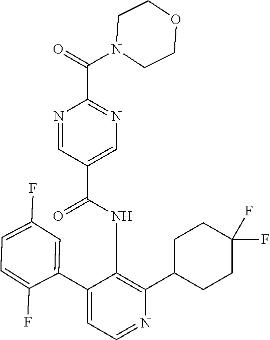 [SMILES: O=C(Nc1c(-c2cc(F)ccc2F)ccnc1C1CCC(F)(F)CC1)c1cnc(C(=O)N2CCOCC2)nc1]

To a mixture of 2-(morpholine-4-carbonyl)pyrimidine-5-carboxylic acid (0.0400 g, 0.169 mmol) and diisopropylethylamine (0.0872 g, 0.675 mmol) in tetrahydrofuran (1.5 mL) was added 2-chloro-1-methyl-pyridin-1-ium iodide (0.0517 g, 0.202 mmol) in one portion at 20° C. The mixture was stirred at 20° C. for 0.5 h then 2-(4,4-difluorocyclohexyl)-4-(2,5-difluorophenyl)pyridin-3-amine (0.0820 g, 0.253 mmol) was added. The mixture was stirred at 70° C. for 12 h. The mixture was cooled to 20° C. and poured into water (10 mL). The mixture was extracted with ethyl acetate (10 mL×3). The combined organic layer was washed with brine (10 mL), dried over anhydrous sodium sulfate, filtered and the filtrate was evaporated under reduced pressure. The residue was purified by flash silica gel chromatography, eluting with ethyl acetate, follow by preparative HPLC, eluting with a gradient of 1-30% of ethanol in hexanes, to afford the title compound as a colorless solid (0.0180 g, 60% yield): ¹H NMR (400 MHz, DMSO-d₆) δ 10.52 (s, 1H), 9.11 (s, 2H), 8.65 (d, J=4.8 Hz, 1H), 7.43-7.33 (m, 2H), 7.33-7.23 (m, 2H), 3.68 (s, 4H), 3.53 (t, J=4.4 Hz, 2H), 3.28-3.17 (m, 3H), 2.14-2.04 (m, 2H), 2.04-1.77 (m, 6H); MS (ES+) m/z 544.1 (M+1).

### Example 184-185

In a similar manner as described in Example 183, utilizing the appropriately substituted starting materials and intermediates, the following compounds were prepared:

| Example | | | MS (ES+) | |
|---|---|---|---|---|
| No. | Structure | Yield | m/z | ¹H-NMR |
| 184 | 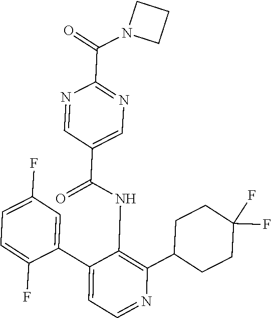 [SMILES: O=C(Nc1c(-c2cc(F)ccc2F)ccnc1C1CCC(F)(F)CC1)c1cnc(C(=O)N2CCC2)nc1] | 13% | 514.1 (M + 1) | ¹H NMR (400 MHz, DMSO-d₆) δ 10.54 (s, 1H), 9.10 (s, 2H), 8.64 (d, J = 4.8 Hz, 1H), 7.39 (d, J = 5.2 Hz, 1H), 7.35 (td, J = 8.8, 4.4 Hz, 1H), 7.31-7.22 (m, 2H), 4.39 (t, J = 7.6 Hz, 2H), 4.10 (t, J = 7.6 Hz, 2H), 3.28- 3.17 (m, 1H), 2.32-2.22 (m, 2H), 2.172-2.05 (m, 2H), 2.04-1.74 (m, 6H) |
| | 2-(azetidine-1- | | | |
| | carbonyl)-N-(2- | | | |
| | (4,4- | | | |
| | difluorocyclo- | | | |
| | hexyl)-4-(2,5- | | | |
| | difluorophenyl) | | | |
| | pyridin-3- | | | |
| | yl)pyrimidine-5- | | | |
| | carboxamide | | | |
| 185 | 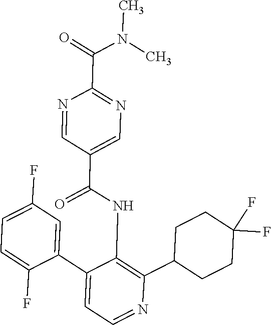 [SMILES: CN(C)C(=O)c1ncc(C(=O)Nc2c(-c3cc(F)ccc3F)ccnc2C2CCC(F)(F)CC2)cn1] | 15% | 502.0 (M + 1) | ¹H NMR (400 MHz, DMSO-d₆) δ 10.50 (s, 1H), 9.09 (s, 2H), 8.64 (d, J = 4.8 Hz, 1H), 7.39 (d, J = 4.8 Hz, 1H), 7.35 (dd, J = 8.8, 4.4 Hz, 1H), 7.31-7.24 (m, 2H), 3.32-3.23 (m, 1H), 3.03 (s, 3H), 2.80 (s, 3H), 1.93-1.92 (m, 4H), 1.91-1.83 (m, 4H) |
| | N⁵-[2-(4,4- | | | |
| | difluorocyclo- | | | |
| | hexyl)-4-(2,5- | | | |
| | difluorophenyl)- | | | |
| | 3-pyridyl]-N²,N²- | | | |
| | dimethyl- | | | |
| | pyrimidine-2,5- | | | |
| | dicarboxamide | | | |

### Example 186

### Synthesis of N-(2-(4,4-difluorocyclohexyl)-4-(2,5-difluorophenyl)pyridin-3-yl)-2-(dimethylamino)acetamide

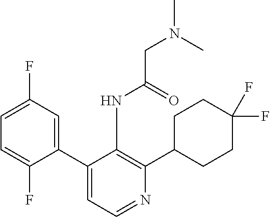 [SMILES: CN(C)CC(=O)Nc1c(-c2cc(F)ccc2F)ccnc1C1CCC(F)(F)CC1]

### Step 1. Preparation of 2-chloro-N-(2-(4,4-difluorocyclohexyl)-4-(2,5-difluorophenyl)pyridin-3-yl)acetamide

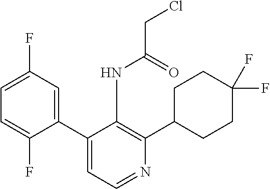 [SMILES: O=C(CCl)Nc1c(-c2cc(F)ccc2F)ccnc1C1CCC(F)(F)CC1]

To a solution of 2-(4,4-difluorocyclohexyl)-4-(2,5-difluorophenyl)pyridin-3-amine (0.100 g, 0.308 mmol), triethylamine (0.0945 g, 0.934 mmol) and N,N-dimethylpyridin-4-amine (0.00400 g, 0.0327 mmol) in dichloromethane (4 mL) was added 2-chloroacetyl chloride (0.0420 g, 0.372 mmol). The mixture was stirred at 25° C. for 12 h. Then triethylamine (0.156 g, 1.54 mmol) and 2-chloroacetyl chloride (0.175 g, 1.55 mmol) were added. The resulting mixture was stirred at 25° C. for 2 h. The reaction was quenched with water (5 mL). The organic solvent was removed under reduced pressure. The aqueous phase was extracted with ethyl acetate (2×10 mL). The combined extracts were washed with brine (10 mL), dried over sodium sulfate and filtered. The filtrate was concentrated under reduced pressure. The residue was purified by preparative HPLC, eluting with a gradient of 37-67% of acetonitrile in water containing 0.225% formic acid, to afford the title compound as a yellow solid (0.0650 g, 53% yield): ¹H NMR (400 MHz, CDCl₃) δ 8.66 (d, J=4.8 Hz, 1H), 8.00 (s, 1H), 7.22-7.09 (m, 3H), 7.06-6.99 (m, 1H), 4.01 (s, 2H), 2.99-2.86 (m, 1H), 2.32-2.19 (m, 2H), 2.19-2.05 (m, 2H), 1.97-1.73 (m, 5H); MS (ES+) m/z 401.1, 403.1 (M+1).

### Step 2. Preparation of N-(2-(4,4-difluorocyclohexyl)-4-(2,5-difluorophenyl)pyridin-3-yl)-2-(dimethylamino)acetamide

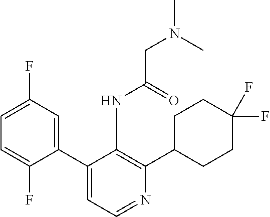 [SMILES: CN(C)CC(=O)Nc1c(-c2cc(F)ccc2F)ccnc1C1CCC(F)(F)CC1]

A mixture of 2-chloro-N-(2-(4,4-difluorocyclohexyl)-4-(2,5-difluorophenyl)pyridin-3-yl)acetamide (0.0650 g, 0.162 mmol), dimethylamine (0.0270 g, 0.331 mmol, hydrochloride) and potassium carbonate (0.113 g, 0.818 mmol) in acetonitrile (5 mL) was stirred at 50° C. for 2 h. The reaction mixture was cooled to ambient temperature and concentrated under reduced pressure. Ethyl acetate (10 mL) and water (10 mL) were added, and the layers were separated. The aqueous phase was extracted with ethyl acetate (2×10 mL). The combined extracts were washed with brine (10 mL), dried over sodium sulfate and filtered. The filtrate was concentrated under reduced pressure. The residue was purified by preparative HPLC, eluting with a gradient of 9-39% of acetonitrile in water containing 0.225% formic acid, to afford the title compound as a colorless solid (0.0296 g, 44% yield): ¹H NMR (400 MHz, MeOD-d₄) δ 8.55 (d, J=4.8 Hz, 1H), 7.29 (d, J=4.8 Hz, 1H), 7.28-7.16 (m, 2H), 7.13 (ddd, J=8.4, 5.6, 3.2 Hz, 1H), 3.14-2.99 (m, 3H), 2.28-2.11 (m, 8H), 2.10-1.97 (m, 2H), 1.97-1.78 (m, 4H); MS (ES+) m/z 410.2, 412.2 (M+1).

### Example 187

### Synthesis of N-(2-(4,4-difluorocyclohexyl)-4-(2,5-difluorophenyl)pyridin-3-yl)-2-morpholinoacetamide

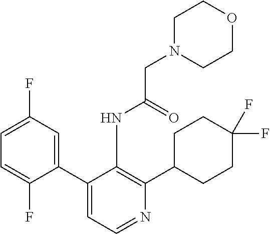 [SMILES: O=C(CN1CCOCC1)Nc1c(-c2cc(F)ccc2F)ccnc1C1CCC(F)(F)CC1]

In a similar manner as described in examples disclosed herein utilizing the appropriately substituted starting materials and intermediates, the title compound was afforded as a colorless solid (0.119 g, 13% yield): ¹H NMR (400 MHz, MeOD-d₄) δ 8.57 (d, J=5.2 Hz, 1H), 7.31 (d, J=5.2 Hz, 1H), 7.29-7.19 (m, 2H), 7.14 (ddd, J=8.4, 5.6, 2.8 Hz, 1H), 3.72-3.61 (m, 4H), 3.13-3.01 (m, 3H), 2.41-2.31 (m, 4H), 2.26-2.12 (m, 2H), 2.05 (d, J=14.0 Hz, 2H), 1.90 (d, J=14.4 Hz, 4H); MS (ES+) m/z 452.1 (M+1).

### Example 188

### Synthesis of 1-(tert-butyl)-3-(2-(4,4-difluorocyclohexyl)-4-(2,5-difluorophenyl)pyridin-3-yl)urea

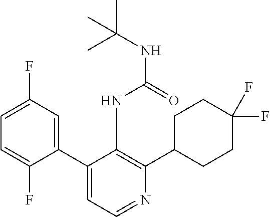 [SMILES: CC(C)(C)NC(=O)Nc1c(-c2cc(F)ccc2F)ccnc1C1CCC(F)(F)CC1]

### Step 1. Preparation of 1-(tert-butyl)-3-(2-chloro-4-(2,5-difluorophenyl)pyridin-3-yl)urea

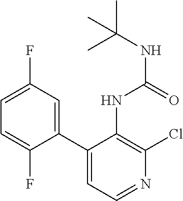 [SMILES: CC(C)(C)NC(=O)Nc1c(-c2cc(F)ccc2F)ccnc1Cl]

A solution of tert-butyl (2-chloro-4-(2,5-difluorophenyl)pyridin-3-yl)carbamate (0.100 g, 0.293 mmol), 2-methylpropan-2-amine (0.0220 g, 0.300 mmol), 4-dimethylaminopyridine (0.0400 g, 0.327 mmol) and 4 Å molecular sieves (0.1 g) in N,N-dimethylformamide (5 mL) was stirred at 25° C. for 1 h under a nitrogen atmosphere. The reaction mixture was warmed to 110° C. and stirred at 110° C. for 12 h. The mixture was cooled to 25° C. The reaction mixture was diluted with water (10 mL) and extracted with ethyl acetate (2×10 mL). The combined organic layers were washed with brine (10 mL), dried over sodium sulfate, filtered, and concentrated under reduced pressure. The residue was purified by flash silica gel chromatography, eluting with a gradient of 50-65% of ethyl acetate in petroleum ether, to afford the title compound as a yellow oil (0.0800 g, 73% yield): ¹H NMR (400 MHz, DMSO-d₆) δ 8.28 (d, J=4.8 Hz, 1H), 7.76 (s, 1H), 7.41 (dd, J=4.8, 1.2 Hz, 1H), 7.35 (dt, J=9.2, 4.4 Hz, 1H), 7.30-7.19 (m, 2H), 6.18 (s, 1H), 1.06 (s, 9H); MS (ES+) m/z 340.1, 342.1 (M+1).

### Step 2. Preparation of 1-(tert-butyl)-3-(2-(4,4-difluorocyclohex-1-en-1-yl)-4-(2,5-difluorophenyl)pyridin-3-yl)urea

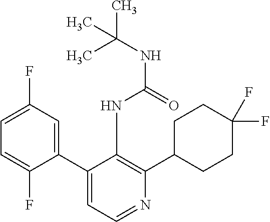 [SMILES: CC(C)(C)NC(=O)Nc1c(-c2cc(F)ccc2F)ccnc1C1CCC(F)(F)CC1]

To a solution of 1-(tert-butyl)-3-(2-chloro-4-(2,5-difluorophenyl)pyridin-3-yl)urea (0.0800 g, 0.235 mmol) and 2-(4,4-difluorocyclohex-1-en-1-yl)-4,4,5,5-tetramethyl-1,3,2-dioxaborolane (0.0630 g, 0.258 mmol) in dioxane (4 mL) and water (1 mL) under a nitrogen atmosphere was added [1,1′-bis(diphenylphosphino)ferrocene]dichloropalladium(II) (0.0170 g, 0.0230 mmol) and potassium carbonate (0.0970 g, 0.701 mmol). The mixture was stirred at 100° C. for 2 h. The reaction mixture was cooled to 25° C. The reaction mixture was diluted with water (10 mL) and extracted with ethyl acetate (2×10 mL). The combined organic layers were washed with brine (10 mL), dried over sodium sulfate, filtered, and concentrated under reduced pressure. The residue was purified by flash silica gel chromatography, eluting with a gradient of 60-75% of ethyl acetate in petroleum ether, to afford the title compound as a yellow oil (0.0330 g, 29% yield): ¹H NMR (400 MHz, DMSO-d₆) δ 8.41 (d, J=4.8 Hz, 1H), 7.35 (dd, J=9.2, 4.4 Hz, 1H), 7.32-7.27 (m, 2H), 7.27-7.21 (m, 2H), 6.00 (s, 1H), 5.77 (s, 1H), 2.70-2.64 (m, 4H), 2.20-2.06 (m, 2H), 1.11 (s, 9H).

### Step 3. Preparation of 1-(tert-butyl)-3-(2-(4,4-difluorocyclohexyl)-4-(2,5-difluorophenyl)pyridin-3-yl)urea

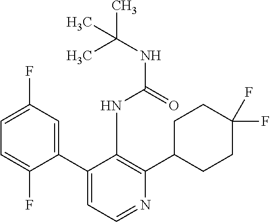 [SMILES: CC(C)(C)NC(=O)Nc1c(-c2cc(F)ccc2F)ccnc1C1CCC(F)(F)CC1]

To a solution of 1-(tert-butyl)-3-(2-(4,4-difluorocyclohex-1-en-1-yl)-4-(2,5-difluorophenyl)pyridin-3-yl)urea (0.0300 g, 0.0710 mmol) in methanol (3 mL) was added palladium on carbon (0.0300 g, 10 wt % purity) under a nitrogen atmosphere. The suspension was degassed and purged with hydrogen 3 times. The mixture was stirred under hydrogen (15 psi, balloon) at 25° C. for 12 h. The resulting mixture was filtered over Celite. The filter cake was washed with methanol (30 mL). The filtrate was concentrated under reduced pressure. The residue was purified by preparative HPLC, eluting with a gradient of 40-70% of acetonitrile in water containing 0.225% formic acid, to afford the title compound as a yellow solid (0.00530 g, 17% yield): ¹H NMR (400 MHz, DMSO-d₆) δ 8.44 (d, J=4.8 Hz, 1H), 7.50-7.41 (m, 1H), 7.34 (td, J=9.2, 4.4 Hz, 1H), 7.31-7.24 (m, 1H), 7.24-7.15 (m, 2H), 6.04-5.95 (m, 1H), 3.10 (d, J=5.2 Hz, 1H), 2.22-2.08 (m, 2H), 1.97-1.74 (i, 6H), 1.12 (H, 9H); MS (ES+) m/z 424.2 (M+1).

### Example 189-192

In a similar manner as described in examples disclosed herein, utilizing the appropriately substituted starting materials and intermediates, the following compounds were prepared:

| Example | Structure | | MS (ES+) | |
|---|---|---|---|---|
| No. | Name | Yield | m/z | ¹H-NMR |
| 189 | 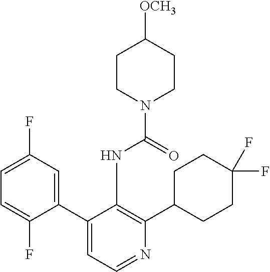 [SMILES: COC1CCN(C(=O)Nc2c(-c3cc(F)ccc3F)ccnc2C2CCC(F)(F)CC2)CC1] | 25% | 466.2 (M + 1) | ¹H NMR (400 MHz, DMSO-d₆) δ 8.48 (d, J = 4.8 Hz, 1H), 8.11 (s, 1H), 7.39-7.26 (m, 2H), 7.24 (dd, J = 4.8, 1.2 Hz, 1H), 7.15 (ddd, J = 8.8, 5.6, 3.2 Hz, 1H), 3.61 (d, J = 13.6 Hz, 2H), 3.29-3.25 (m, 1H), 3.23 (s, 3H), 3.12 (s, 1H), 2.95 (t, J = 10.4 Hz, 2H), 2.13 (d, J = 6.0 Hz, 2H), 1.99-1.75 (m, 6H), 1.70-1.58 (m, 2H), |
| | N-(2-(4,4- | | | 1.18-1.04 (m, 2H) |
| | difluorocyclo- | | | |
| | hexyl)-4-(2,5- | | | |
| | difluoro- | | | |
| | phenyl)pyridin-3- | | | |
| | yl)-4- | | | |
| | methoxypiperidine- | | | |
| | 1-carboxamide | | | |
| 190 | 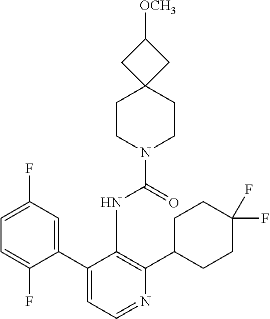 [SMILES: COC1CC2(CCN(C(=O)Nc3c(-c4cc(F)ccc4F)ccnc3C3CCC(F)(F)CC3)CC2)C1] | 19% | 505.2 (M + 1) | ¹H-NMR (400 MHz; DMSO-d₆) δ 8.48 (d, J = 4.9 Hz, 1H), 8.07 (s, 1H), 7.38-7.25 (m, 2H), 7.24 (dd, J = 4.9, 1.2 Hz, 1H), 7.14 (ddd, J = 8.9, 5.7, 3.2 Hz, 1H), 3.83 (quintet, J = 6.9 Hz, 1H), 3.22- 3.14 (m, 5H), 3.10 (s, 3H), 2.14-2.12 (m, 2H), 2.10-2.05 (m, 2H), 1.90-1.81 (m, 6H), 1.57-1.52 (m, 2H), 1.24-1.20 (m, 4H) |
| | N-(2-(4,4- | | | |
| | difluorocyclo- | | | |
| | hexyl)-4-(2,5- | | | |
| | difluorophenyl) | | | |
| | pyridin-3-yl)-2- | | | |
| | methoxy-7- | | | |
| | azaspiro[3.5] | | | |
| | nonane-7- | | | |
| | carboxamide | | | |
| 191 | 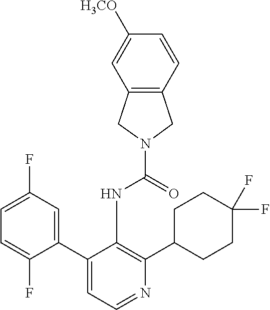 [SMILES: COc1ccc2c(c1)CN(C(=O)Nc1c(-c3cc(F)ccc3F)ccnc1C1CCC(F)(F)CC1)C2] | 33% | 500.2 (M + 1) | ¹H NMR (400 MHz, DMSO-d₆) δ 8.51 (d, J = 4.8 Hz, 1H), 8.01 (s, 1H), 7.37-7.14 (m, 5H), 6.92-6.82 (m, 2H), 4.47 (d, J = 12.0 Hz, 4H), 3.74 (s, 3H), 3.29-3.22 (m, 1H), 2.16-2.05 (m, 2H), 2.00-1.82 (m, 6H) |
| | N-(2-(4,4- | | | |
| | difluorocyclo- | | | |
| | hexyl)-4-(2,5- | | | |
| | difluorophenyl) | | | |
| | pyridin-3-yl)-5- | | | |
| | methoxyiso- | | | |
| | indoline-2- | | | |
| | carboxamide | | | |
| 192 | 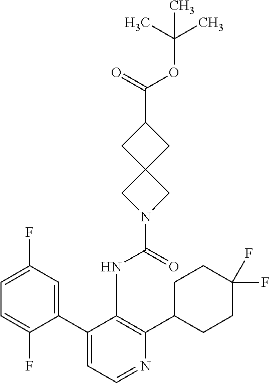 [SMILES: CC(C)(C)OC(=O)C1CC2(C1)CN(C(=O)Nc1c(-c3cc(F)ccc3F)ccnc1C1CCC(F)(F)CC1)C2] | 62% | 549.4 (M + 1) | ¹H NMR (400 MHz, CDCl₃) δ 8.59 (d, J = 4.8 Hz, 1H), 7.21-7.11 (m, 3H), 7.10-7.03 (m, 1H), 5.66 (s, 1H), 3.99 (s, 4H), 3.90 (s, 4H), 3.08 (t, J = 11.6 Hz, 1H), 2.24 (s, 2H), 2.16-2.01 (m, 2H), 1.98-1.79 (m, 4H), 1.44 (s, 9H) |
| | tert-butyl 6-((2- | | | |
| | (4,4- | | | |
| | difluorocyclo- | | | |
| | hexyl)-4-(2,5- | | | |
| | difluorophenyl) | | | |
| | pyridin-3- | | | |
| | yl)carbamoyl)- | | | |
| | 2,6- | | | |
| | diazaspiro[3.3] | | | |
| | heptane-2- | | | |
| | carboxylate | | | |

### Example 193

### Synthesis of N-(2-(4,4-difluorocyclohexyl)-4-(2,5-difluorophenyl)pyridin-3-yl)-2,6-diazaspiro[3.3]heptane-2-carboxamide

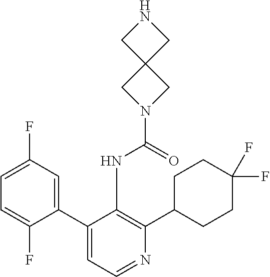 [SMILES: O=C(Nc1c(-c2cc(F)ccc2F)ccnc1C1CCC(F)(F)CC1)N1CC2(CNC2)C1]

To a mixture of tert-butyl 6-((2-(4,4-difluorocyclohexyl)-4-(2,5-difluorophenyl)pyridin-3-yl)carbamoyl)-2,6-diazaspiro[3.3]heptane-2-carboxylate (0.0500 g, 0.0911 mmol) in dichloromethane (1 mL) was added trifluoroacetic acid (0.154 g, 1.35 mmol). The mixture was stirred at 25° C. for 1 h and then saturated sodium bicarbonate (10 mL) was added. The mixture was extracted with ethyl acetate (3×15 mL). The combined organic extracts were dried over anhydrous sodium sulfate, filtered and the filtrate was concentrated in vacuo. The residue was purified by preparative HPLC, eluting with a gradient of 37-67% of acetonitrile in water containing ammonium hydroxide, to afford the title compound as a colorless solid (0.00800 g, 18% yield): ¹H NMR (400 MHz, CDCl₃) δ 8.58 (d, J=4.8 Hz, 1H), 7.21-7.10 (m, 3H), 7.09-7.02 (m, 1H), 5.61 (s, 1H), 3.90 (s, 4H), 3.71 (s, 4H), 3.09 (t, J=11.2 Hz, 1H), 2.23 (d, J=4.0 Hz, 2H), 2.17-2.02 (m, 2H), 1.86-1.84 (m, 4H); MS (ES+) m/z 449.3 (M+1).

### Example 194

### Synthesis of N-(2-(4,4-difluorocyclohexyl)-4-(2,5-difluorophenyl)pyridin-3-yl)-6-methyl-2,6-diazaspiro[3.3]heptane-2-carboxamide

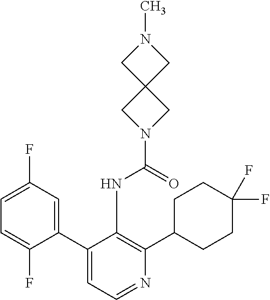 [SMILES: CN1CC2(C1)CN(C(=O)Nc1c(-c3cc(F)ccc3F)ccnc1C1CCC(F)(F)CC1)C2]

To a mixture of N-(2-(4,4-difluorocyclohexyl)-4-(2,5-difluorophenyl)pyridin-3-yl)-2,6-diazaspiro[3.3] heptane-2-carboxamide (0.0600 g, 0.133 mmol) in methanol (2 mL) was added paraformaldehyde (0.0336 g, 0.535 mmol). The mixture was stirred at 25° C. for 0.5 h, then sodium cyanoborohydride (0.0160 g) was added. The mixture was stirred at 25° C. for 12 h. The residue was poured into water (20 mL) and extracted with ethyl acetate (3×15 mL). The combined organic extracts were dried over anhydrous sodium sulfate, filtered, and the filtrate was concentrated in vacuo. The residue was purified by preparative HPLC, eluting with a gradient of 27-57% of acetonitrile in water containing 0.05% ammonium hydroxide, to afford the title compound as a colorless solid (0.0192 g, 30% yield): ¹H NMR (400 MHz, CDCl₃) δ 8.58 (d, J=4.8 Hz, 1H), 7.21-7.09 (m, 3H), 7.06 (ddd, J=8.4, 5.6, 2.8 Hz, 1H), 5.59 (s, 1H), 3.85 (s, 4H), 3.26 (s, 4H), 3.16-3.03 (m, 1H), 2.31-2.27 (m, 3H), 2.26-2.19 (m, 2H), 2.16-2.02 (m, 2H), 1.97-1.75 (m, 4H); MS (ES+) m/z 463.3 (M+1).

### Example 195

### Synthesis of N-(2-(4,4-difluorocyclohexyl)-4-(2,5-difluorophenyl)pyridin-3-yl)-2-(2-hydroxypropan-2-yl)pyrimidine-5-carboxamide

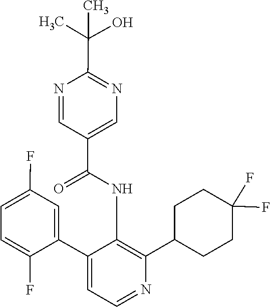 [SMILES: CC(C)(O)c1ncc(C(=O)Nc2c(-c3cc(F)ccc3F)ccnc2C2CCC(F)(F)CC2)cn1]

### Step 1. Preparation of 2-(5-bromopyrimidin-2-yl)propan-2-ol

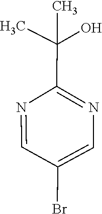

To a solution of methyl 5-bromopyrimidine-2-carboxylate (2.00 g, 9.22 mmol) in dichloromethane (40 mL) was added dropwise methylmagnesium bromide (3.00 M in tetrahydrofuran, 12.3 mL) at 0° C. The reaction mixture was stirred at 0° C. for 30 min. The reaction mixture was slowly warmed up to 20° C. and stirred at 20° C. for 12 h. The reaction was quenched with saturated ammonium chloride solution (20 mL) at 0° C. The mixture was diluted with ethyl acetate (30 mL) and water (30 mL). The layers were separated, and the aqueous phase was extracted with ethyl acetate (2×30 mL). The organic layer was washed with brine (30 mL) and dried over anhydrous sodium sulfate, filtered and concentrated in vacuo. The residue was purified by flash silica gel chromatography, eluting with a gradient of 0-30% of ethyl acetate in petroleum ether to afford the title compound as a yellow solid (1.10 g, 53% yield); ¹H NMR (400 MHz, DMSO-d₆) δ 8.98 (s, 2H), 5.14 (s, 1H), 1.49 (s, 6H).

### Step 2. Preparation of methyl 2-(2-hydroxypropan-2-yl)pyrimidine-5-carboxylate

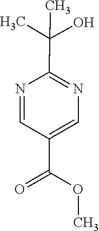

To a solution of 2-(5-bromopyrimidin-2-yl)propan-2-ol (1.40 g, 6.45 mmol) in methanol (40 mL) was added triethylamine (2.00 g, 19.8 mmol) and [1,1′-bis(diphenylphosphino)ferrocene]dichloropalladium(II) (0.476 g, 0.651 mmol) in one portion. The solution was stirred at 80° C. under an atmosphere of carbon monoxide (50 psi) for 16 h. The resulting mixture was cooled to 20° C. and filtered over a pad of Celite. The filtrate was concentrated under reduced pressure. The residue was purified by flash silica gel chromatography, eluting with a gradient of 0-40% of ethyl acetate in petroleum ether to afford the title compound as a white solid (1.00 g, 78% yield); ¹H NMR (400 MHz, DMSO-d₆) δ 9.23 (s, 2H), 5.26 (s, 1H), 3.91 (s, 3H), 1.51 (s, 6H).

### Step 3. Preparation of 2-(2-hydroxypropan-2-yl)pyrimidine-5-carboxylic acid

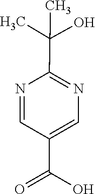

To a solution of methyl 2-(2-hydroxypropan-2-yl)pyrimidine-5-carboxylate (0.400 g, 2.04 mmol) in tetrahydrofuran (10 mL) was added a solution of lithium hydroxide hydrate (0.428 g, 10.2 mmol) in water (10 mL) at 0° C. The reaction mixture was stirred at 15° C. for 1 h. The residue was diluted with ethyl acetate (30 mL) and water (30 mL). The layers were separated, and the aqueous phase was acidified to pH=4 with 1 M hydrochloric acid. The aqueous phase was extracted with ethyl acetate (3×30 mL). The organic layer was washed with brine (30 mL) and dried over anhydrous sodium sulfate, filtered and concentrated in vacuo to afford 2-(2-hydroxypropan-2-yl)pyrimidine-5-carboxylic acid as a colorless solid (0.0500 g, 13% yield): ¹H NMR (400 MHz, DMSO-d₆) δ 9.19 (s, 2H), 5.24 (br s, 1H), 1.50 (s, 6H).

### Step 4. Preparation of N-(2-(4,4-difluorocyclohexyl)-4-(2,5-difluorophenyl)pyridin-3-yl)-2-(2-hydroxypropan-2-yl)pyrimidine-5-carboxamide

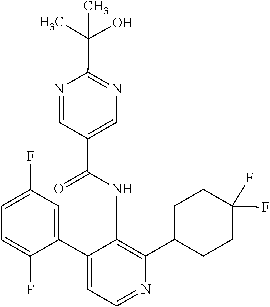 [SMILES: CC(C)(O)c1ncc(C(=O)Nc2c(-c3cc(F)ccc3F)ccnc2C2CCC(F)(F)CC2)cn1]

In a similar manner as described in examples disclosed herein utilizing the appropriately substituted starting materials and intermediates, the title compound was afforded as a colorless solid (0.0440 g, 54% yield): ¹H NMR (400 MHz, DMSO-d₆) δ 10.40 (s, 1H), 9.01 (s, 2H), 8.63 (d, J=4.8 Hz, 1H), 7.42-7.32 (m, 2H), 7.26 (dd, J=8.0, 2.8 Hz, 2H), 5.20 (s, 1H), 3.27-3.14 (m, 1H), 2.20-2.04 (m, 2H), 2.03-1.73 (m, 6H), 1.50 (s, 6H); MS (ES+) m/z 489.2 (M+1).

### Example 196

### Synthesis of N-(2-(4,4-difluorocyclohexyl)-4-(2,5-difluorophenyl)pyridin-3-yl)-2-(2-methoxypropan-2-yl)pyrimidine-5-carboxamide

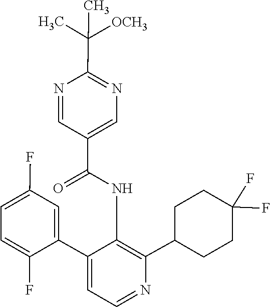 [SMILES: COC(C)(C)c1ncc(C(=O)Nc2c(-c3cc(F)ccc3F)ccnc2C2CCC(F)(F)CC2)cn1]

### Step 1. Preparation of methyl 2-(2-methoxypropan-2-yl)-pyrimidine-5-carboxylate

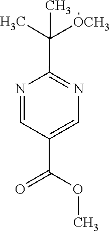

To a solution of sodium hydride (0.245 g, 6.13 mmol, 60% purity) in dimethylformamide (8 mL) was added methyl 2-(2-hydroxypropan-2-yl)pyrimidine-5-carboxylate (0.400 g, 2.04 mmol) in dimethylformamide (3 mL) at 15° C. under nitrogen atmosphere. The reaction mixture was stirred at 50° C. for 0.5 h. To the mixture was added dropwise iodomethane (0.868 g, 6.12 mmol) and the reaction mixture was stirred at 50° C. for 2 h. The reaction mixture was added dropwise to 1 M formic acid aqueous solution (10 mL) at 0° C., and then diluted with ethyl acetate (20 mL). The organic layer was washed with brine (25 mL), dried over anhydrous sodium sulfate, filtered and concentrated under reduced pressure. The residue was purified by flash silica gel chromatography, eluting with a gradient of 0-40% of ethyl acetate in petroleum ether, to afford the title compound as a colorless solid (0.240 g, 56% yield): ¹H NMR (400 MHz, DMSO-d₆) δ 9.25 (s, 2H), 3.91 (s, 3H), 3.01 (s, 3H), 1.55 (s, 6H).

### Step 2. Preparation of 2-(2-methoxypropan-2-yl)pyrimidine-5-carboxylic acid

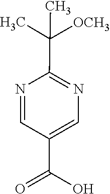

To a solution of methyl 2-(2-methoxypropan-2-yl)pyrimidine-5-carboxylate (0.200 g, 0.951 mmol) in tetrahydrofuran (3 mL) was added lithium hydroxide monohydrate (0.0800 g, 1.91 mmol) in water (3 mL) at 0° C. and the reaction mixture was stirred at 15° C. for 1 h. The mixture was diluted with ethyl acetate (15 mL) and water (15 mL). The layers were separated, and the aqueous phase was acidified to pH=3 with 1 M hydrochloric acid. The aqueous phase was extracted with ethyl acetate (2×20 mL). The organic layer was washed with brine (25 mL) and dried over anhydrous sodium sulfate, filtered and concentrated in vacuo to afford the title compound as a colorless solid (0.120 g, 64% yield): ¹H NMR (400 MHz, DMSO-d₆) δ 13.77 (br s, 1H), 9.22 (s, 2H), 3.01 (s, 3H), 1.55 (s, 6H).

### Step 3. Preparation of N-(2-(4,4-difluorocyclohexyl)-4-(2,5-difluorophenyl)pyridin-3-yl)-2-(2-methoxypropan-2-yl)pyrimidine-5-carboxamide

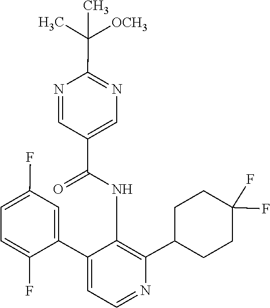 [SMILES: COC(C)(C)c1ncc(C(=O)Nc2c(-c3cc(F)ccc3F)ccnc2C2CCC(F)(F)CC2)cn1]

In a similar manner as described in examples disclosed herein utilizing the appropriately substituted starting materials and intermediates, the title compound was afforded as a colorless solid (0.209 g, 79% yield): ¹H NMR (400 MHz, DMSO-d₆) δ 10.44 (s, 1H), 9.02 (s, 2H), 8.63 (d, J=4.8 Hz, 1H), 7.43-7.33 (m, 2H), 7.33-7.21 (m, 2H), 3.28-3.14 (m, 1H), 3.02 (s, 3H), 2.16-2.04 (m, 2H), 2.03-1.76 (m, 6H), 1.54 (s, 6H); MS (ES+) m/z 503.2 (M+1).

### Example 197

### Synthesis of N-(2-(4,4-difluorocyclohexyl)-4-(2,5-difluorophenyl)pyridin-3-yl)-2-(2-fluoropropan-2-yl)pyrimidine-5-carboxamide

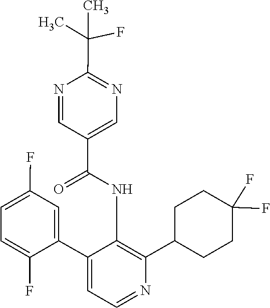 [SMILES: CC(C)(F)c1ncc(C(=O)Nc2c(-c3cc(F)ccc3F)ccnc2C2CCC(F)(F)CC2)cn1]

### Step 1. Preparation of methyl 2-(2-fluoropropan-2-yl)pyrimidine-5-carboxylate

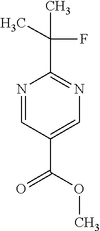

To a solution of methyl 2-(1-hydroxy-1-methyl-ethyl)pyrimidine-5-carboxylate (0.250 g, 1.27 mmol) in dichloromethane (10 mL) was added N,N-diethyl-1,1,1-trifluoro-λ⁴-sulfanamine (0.616 g, 3.82 mmol) dropwise at −65° C. under nitrogen atmosphere. The reaction mixture was stirred at 15° C. for 16 h. To the mixture was added saturated sodium bicarbonate solution (20 mL) at 0° C. The solution was extracted with dichloromethane (3×10 mL). The combined organic extracts were washed with brine (2×20 mL) and then dried over anhydrous sodium sulfate, filtered and concentrated in vacuo The residue was purified by flash silica gel chromatography, eluting with a gradient of 0-40% of ethyl acetate in petroleum ether, to afford the title compound as a colorless solid (0.210 g, 82% yield): ¹H NMR (400 MHz, DMSO-d₆) δ 9.28 (s, 2H), 3.92 (s, 3H), 1.77 (s, 3H), 1.71 (s, 3H).

### Step 2. Preparation of 2-(2-methoxypropan-2-yl)pyrimidine-5-carboxylic acid

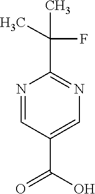

In a similar manner as described in examples disclosed herein, utilizing the appropriately substituted starting materials and intermediates, the title compound was afforded as a colorless solid (0.130 g, 69% yield) as a white solid: ¹H NMR (400 MHz, DMSO-d₆) δ 9.26 (s, 2H), 1.77 (s, 3H), 1.72 (s, 3H).

### Step 3. Preparation of N-(2-(4,4-difluorocyclohexyl)-4-(2,5-difluorophenyl)pyridin-3-yl)-2-(2-fluoropropan-2-yl)pyrimidine-5-carboxamide

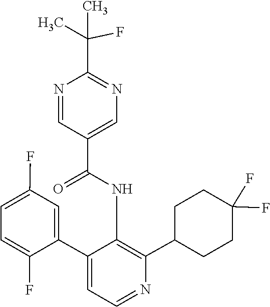 [SMILES: CC(C)(F)c1ncc(C(=O)Nc2c(-c3cc(F)ccc3F)ccnc2C2CCC(F)(F)CC2)cn1]

In a similar manner as described in examples disclosed herein, utilizing the appropriately substituted starting materials and intermediates, the title compound was afforded as a colorless solid (0.125 g, 0.242 mmol): ¹H NMR (400 MHz, DMSO-d₆) δ 10.47 (s, 1H), 9.06 (s, 2H), 8.64 (d, J=4.8 Hz, 1H), 7.42-7.33 (m, 2H), 7.32-7.21 (m, 2H), 3.27-3.12 (m, 1H), 2.15-2.05 (m, 2H), 2.04-1.79 (m, 6H), 1.76 (s, 3H), 1.71 (s, 3H); MS (ES+) m/z 491.3 (M+1).

### Example 198

### Synthesis of N-(2-(4,4-difluorocyclohexyl)-4-(2,5-difluorophenyl)pyridin-3-yl)-2-(2-fluoropropan-2-yl)pyrimidine-5-carboxamide

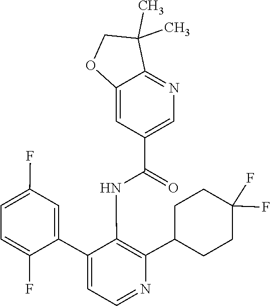 [SMILES: CC1(C)COc2cc(C(=O)Nc3c(-c4cc(F)ccc4F)ccnc3C3CCC(F)(F)CC3)cnc21]

### Step 1. Preparation of methyl 5-((2-methylallyl)oxy)nicotinate

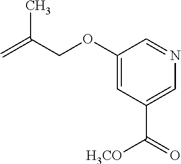

To a mixture of methyl 5-hydroxynicotinate (4.00 g, 26.1 mmol) and 2-methylprop-2-en-1-ol (2.07 g, 28.7 mmol) in tetrahydrofuran (100 mL) was added triphenylphosphine (8.22 g, 31.3 mmol) under a nitrogen atmosphere. The mixture was stirred at 25° C. for 1 h and then diethyl (E)-diazene-1,2-dicarboxylate (5.46 g, 31.3 mmol) was added at 0° C. The mixture was stirred at 25° C. for 12 h. The residue was poured into water (30 mL) and extracted with ethyl acetate (3×30 mL). The combined organic extracts were dried over anhydrous sodium sulfate, filtered and the filtrate was concentrated in vacuo. The residue was purified by flash silica gel chromatography, eluting with a gradient of 33% of ethyl acetate in petroleum ether, to afford the title compound as a yellow oil (2.0 g, 37% yield): ¹H NMR (400 MHz, CDCl₃) δ 8.82 (s, 1H), 8.50 (d, J=2.8 Hz, 1H), 7.79-7.75 (m, 1H), 5.12 (s, 1H), 5.05 (s, 1H), 4.53 (s, 2H), 3.96 (s, 3H), 1.84 (s, 3H).

### Step 2. Preparation of methyl 3,3-dimethyl-2,3-dihydrofuro[3,2-b]pyridine-6-carboxylate

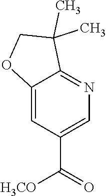

To a mixture of methyl methyl 5-((2-methylallyl)oxy)nicotinate (1.00 g, 4.83 mmol) in ethanol (10 mL) was added trifluoroacetic acid (1.10 g, 9.65 mmol). The mixture was stirred at 25° C. for 1 h, then tris [(Z)-1-methyl-3-oxo-but-1-enoxy] iron (0.852 g, 2.41 mmol), phenylsilane (1.31 g, 12.1 mmol) and 2-tert-butylperoxy-2-methyl-propane (2.12 g, 14.5 mmol) were added under a nitrogen atmosphere. The mixture was stirred at 110° C. in sealed tube for 24 h. After cooling to ambient temperature, the reaction mixture was concentrated under reduced pressure. The residue was poured into water (20 mL) and extracted with ethyl acetate (3×15 mL). The combined organic extracts were dried over anhydrous sodium sulfate, filtered and the filtrate was concentrated in vacuo The residue was purified by flash silica gel chromatography, eluting with a gradient of 33% of ethyl acetate in petroleum ether, to afford the title compound as a yellow oil (0.300 g, 12% yield) as yellow oil: ¹H NMR (400 MHz, CDCl₃) δ 8.66 (s, 1H), 8.27 (s, 1H), 4.28 (s, 2H), 3.94 (s, 3H), 1.51 (s, 6H).

### Step 3. Preparation of 3,3-dimethyl-2,3-dihydrofuro[3,2-b]pyridine-6-carboxylic acid

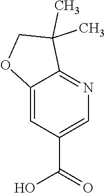

To a mixture of methyl 3,3-dimethyl-2,3-dihydrofuro[3,2-b]pyridine-6-carboxylate (0.250 g, 1.21 mmol) in methanol (1.2 mL) was added a solution of sodium hydroxide (0.0965 g, 2.41 mmol) in water (1.2 mL). The mixture was stirred at 25° C. for 4 h. The reaction mixture was poured into water (10 mL) and adjusted to pH=2 with hydrochloric acid (2 M). The mixture was lyophilized to afford 3,3-dimethyl-2,3-dihydrofuro[3,2-b]pyridine-6-carboxylic acid as a black-brown solid (0.310 g, crude): ¹H NMR (400 MHz, DMSO-d₆) δ 8.57 (s, 1H), 8.44 (s, 1H), 4.35 (s, 2), 1.46 (s, 6H).

### Step 4. Preparation of N-(2-(4,4-difluorocyclohexyl)-4-(2,5-difluorophenyl)pyridin-3-yl)-2-(2-fluoropropan-2-yl)pyrimidine-5-carboxamide

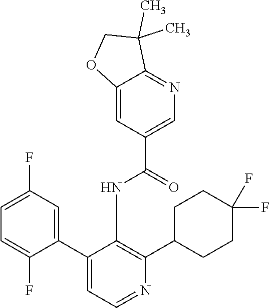 [SMILES: CC1(C)COc2cc(C(=O)Nc3c(-c4cc(F)ccc4F)ccnc3C3CCC(F)(F)CC3)cnc21]

In a similar manner as described in examples disclosed herein, utilizing the appropriately substituted starting materials and intermediates, the title compound was afforded as a colorless solid (0.125 g, 0.242 mmol): ¹H NMR (400 MHz, CDCl₃) δ 8.70 (d, J=4.8 Hz, 1H), 8.26 (s, 1H), 8.11 (s, 1H), 7.47 (s, 1H), 7.23-7.21 (m, 1H), 7.20-7.14 (m, 1H), 7.14-7.07 (m, 2H), 4.23 (s, 2H), 3.19-3.08 (m, 1H), 2.35-2.10 (m, 5H), 1.94 (s, 2H), 1.87-1.82 (m, 1H), 1.35 (s, 6H); MS (ES+) m/z 500.4 (M+1).

### Example 199

### Synthesis of N-(2-(4,4-difluorocyclohexyl)-4-(2,5-difluorophenyl)pyridin-3-yl)-2-(1-(hydroxymethyl)cyclopropyl)acetamide

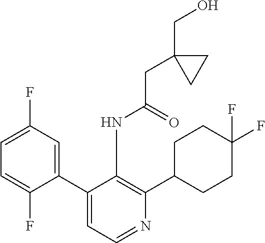 [SMILES: O=C(CC1(CO)CC1)Nc1c(-c2cc(F)ccc2F)ccnc1C1CCC(F)(F)CC1]

### Step 1. Synthesis of 2-(1-((benzyloxy)methyl)cyclopropyl)acetonitrile

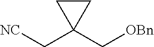 [SMILES: N#CCC1(COCc2ccccc2)CC1]

To a solution of 2-(1-(hydroxymethyl)cyclopropyl)acetonitrile (0.500 g, 4.50 mmol) in tetrahydrofuran (5 mL) was added sodium hydride (0.270 g, 6.75 mmol, 60% purity) at 0° C. under an atmosphere of nitrogen. The mixture was stirred at 25° C. for 0.5 h, then (bromomethyl)benzene (1.54 g, 9.00 mmol) was added at 25° C. The mixture was stirred at 25° C. for 12 h. The mixture was diluted with ethyl acetate (50 mL) and washed with saturated sodium bicarbonate (3×50 mL), dried over sodium sulfate, filtered. The filtrate was concentrated under reduced pressure The residue was purified by flash silica gel chromatography, eluting with a gradient of 0-3% of ethyl acetate in petroleum ether, to afford the title compound as a colorless solid (0.660 g, 69% yield): ¹H NMR (400 MHz, DMSO-d₆) δ 7.40-7.32 (m, 4H), 7.32-7.24 (m, 1H), 4.50 (s, 2H), 3.34 (s, 2H), 2.64 (s, 2H), 0.61-0.46 (m, 4H).

### Step 2. Synthesis of 2-(1-((benzyloxy)methyl)cyclopropyl)acetic acid

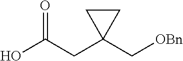 [SMILES: O=C(O)CC1(COCc2ccccc2)CC1]

To a solution of 2-(1-((benzyloxy)methyl)cyclopropyl)acetonitrile (0.560 g, 2.78 mmol) in ethanol (6 mL) was added potassium hydroxide (1.56 g, 27.9 mmol) in water (6 mL) at 25° C. The mixture was stirred at 80° C. for 12 h. The mixture was poured into aqueous 1 M hydrochloric acid (50 mL). The mixture was extracted with ethyl acetate (3×50 mL). The combined organic layers were washed with brine (50 mL), dried over sodium sulfate and filtered. The filtrate was evaporated under reduced pressure to give the title compound as a colorless oil (0.490 g, 68% yield): ¹H NMR (400 MHz, CDCl₃) δ 7.44-7.28 (m, 5H), 4.58 (s, 2H), 3.40 (s, 2H), 2.51 (s, 2H), 0.62-0.55 (m, 4H).

### Step 3. Preparation of 2-(1-((benzyloxy)methyl)cyclopropyl)-N-(2-(4,4-difluorocyclohexyl)-4-(2,5-difluorophenyl)pyridin-3-yl)acetamide

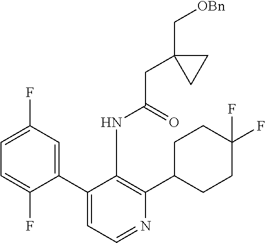 [SMILES: O=C(CC1(COCc2ccccc2)CC1)Nc1c(-c2cc(F)ccc2F)ccnc1C1CCC(F)(F)CC1]

In a similar manner as described in examples disclosed herein, utilizing the appropriately substituted starting materials and intermediates, the title compound was afforded as a colorless solid (0.240 g, 48% yield): ¹H NMR (400 MHz, DMSO-d₆) δ 9.44 (s, 1H), 8.53 (d, J=4.8 Hz, 1H), 7.36-7.24 (m, 8H), 7.15 (ddd, J=8.8, 5.6, 3.2 Hz, 1H), 4.46-4.37 (m, 2H), 3.37-3.28 (m, 4H), 3.10-3.05 (m, 1H), 2.23 (s, 1H), 2.17-2.06 (m, 2H), 1.92-1.70 (m, 5H), 0.45 (d, J=4.4 Hz, 2H), 0.37 (d, J=12.0 Hz, 2H); MS (ES+) m/z 527.3 (M+1).

### Step 4. Preparation of N-(2-(4,4-difluorocyclohexyl)-4-(2,5-difluorophenyl)pyridin-3-yl)-2-(1-(hydroxymethyl)cyclopropyl)acetamide

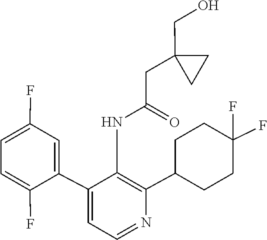 [SMILES: O=C(CC1(CO)CC1)Nc1c(-c2cc(F)ccc2F)ccnc1C1CCC(F)(F)CC1]

To a solution of 2-(1-((benzyloxy)methyl)cyclopropyl)-N-(2-(4,4-difluorocyclohexyl)-4-(2,5-difluorophenyl)pyridin-3-yl)acetamide (0.0400 g, 0.0760 mmol) in methanol (4 mL) was added palladium on carbon (0.0200 g, 10 wt %) at 25° C. under a nitrogen atmosphere. The suspension was degassed and purged with hydrogen three times. The mixture was stirred at 25° C. for 12 h under hydrogen (15 psi, balloon). After completion of the reaction, the resulting mixture was filtered over Celite. The filter cake was washed with methanol (30 mL). The filtrate was concentrated under reduced pressure. The residue was purified by preparative HPLC, eluting with a gradient of 39-59% of acetonitrile in water containing 0.225% formic acid, to afford the title compound as a colorless solid (0.0152 mg, 45% yield): ¹H NMR (400 MHz, DMSO-d₆) δ 9.48 (s, 1H), 8.53 (d, J=4.4 Hz, 1H), 7.41-7.21 (m, 3H), 7.14 (s, 1H), 4.53 (s, 1H), 3.13 (s, 3H), 2.25-2.07 (m, 4H), 1.98-1.71 (m, 6H), 0.31 (d, J=16.0 Hz, 4H); MS (ES+) m/z 437.2 (M+1).

### Example 200

### Synthesis of N-(4-(4,4-difluorocyclohexyl)-6-(2,5-difluorophenyl)-2-methylpyrimidin-5-yl)-5-fluoro-6-methoxynicotinamide

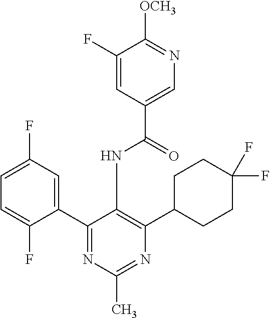 [SMILES: COc1ncc(C(=O)Nc2c(-c3cc(F)ccc3F)nc(C)nc2C2CCC(F)(F)CC2)cc1F]

### Step 1. Preparation of 4-chloro-6-(2,5-difluorophenyl)-2-methylpyrimidin-5-amine

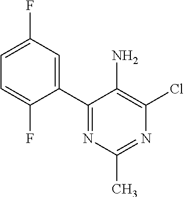 [SMILES: Cc1nc(Cl)c(N)c(-c2cc(F)ccc2F)n1]

To a solution of 4,6-dichloro-2-methylpyrimidin-5-amine (0.200 g, 1.12 mmol), (2,5-difluorophenyl)boronic acid (0.159 g, 1.01 mmol) and potassium carbonate (0.310 g, 2.24 mmol) in dimethyl formamide (5 mL) and water (1 mL) under a nitrogen atmosphere was added tetrakis(triphenylphosphine)palladium(0) (0.130 g, 0.112 mmol). The mixture was stirred at 80° C. The mixture was cooled to 25° C. and concentrated under reduced pressure. The residue was purified by preparative HPLC, eluting with a gradient of 25-55% of acetonitrile in water containing 0.225% formic acid, to afford the title compound as a colorless solid (0.0370 g, 12% yield): ¹H NMR (400 MHz, DMSO-d₆) δ 7.41-7.37 (m, 2H), 7.36-7.31 (m, 1H), 5.43 (s, 2H), 2.45 (s, 3H); MS (ES+) m/z 256.0, 258.0 (M+1).

### Step 2. Preparation of 4-(4,4-difluorocyclohex-1-en-1-yl)-6-(2,5-difluorophenyl)-2-methylpyrimidin-5-amine

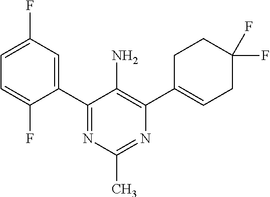 [SMILES: Cc1nc(C2=CCC(F)(F)CC2)c(N)c(-c2cc(F)ccc2F)n1]

To a solution of 4-chloro-6-(2,5-difluorophenyl)-2-methylpyrimidin-5-amine (0.0370 g, 0.144 mmol), 2-(4,4-difluorocyclohex-1-en-1-yl)-4,4,5,5-tetramethyl-1,3,2-dioxaborolane (0.0390 g, 0.159 mmol) and potassium carbonate (0.0600 g, 0.434 mmol) in dioxane (4 mL) and water (1 mL) under a nitrogen atmosphere was added [1,1′-bis(diphenylphosphino)ferrocene]dichloropalladium(II) (0.0110 g, 0.0150 mmol). The mixture was stirred at 100° C. for 2 h. The mixture was cooled to 25° C. The mixture was concentrated under reduced pressure. The residue was purified by flash silica gel chromatography, eluting with a gradient of 30-50% of ethyl acetate in petroleum ether, to afford the title compound as a yellow oil (0.0170 g, 31% yield): ¹H NMR (400 MHz, DMSO-d₆) δ 7.41-7.35 (m, 2H), 7.34-7.29 (m, 1H), 6.03 (s, 1H), 4.92-4.70 (m, 2H), 2.78-2.72 (m, 2H), 2.61 (s, 2H), 2.44 (s, 3H), 2.19 (td, J=7.2, 14.0 Hz, 2H); MS (ES+) m/z 338.1 (M+1).

### Step 3. Preparation of 4-(4,4-difluorocyclohexyl)-6-(2,5-difluorophenyl)-2-methylpyrimidin-5-amine

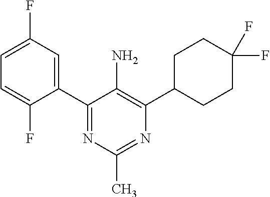 [SMILES: Cc1nc(-c2cc(F)ccc2F)c(N)c(C2CCC(F)(F)CC2)n1]

To a solution of 4-(4,4-difluorocyclohex-1-en-1-yl)-6-(2,5-difluorophenyl)-2-methylpyrimidin-5-amine (0.0170 g, 0.0500 mmol) in methanol (3 mL) was added palladium on carbon (0.0170 g, 10 wt %) under a nitrogen atmosphere. The suspension was degassed and purged with hydrogen 3 times. The mixture was stirred under hydrogen (15 psi, balloon) at 25° C. for 1 h. After completion of the reaction, the resulting mixture was filtered over Celite. The filter cake was washed with methanol (15 mL). The filtrate was concentrated under reduced pressure to afford the title compound as a yellow oil (0.0120 g, 63% yield): MS (ES+) m/z 340.1 (M+1).

### Step 4. Preparation of N-(4-(4,4-difluorocyclohexyl)-6-(2,5-difluorophenyl)-2-methylpyrimidin-5-yl)-5-fluoro-6-methoxynicotinamide

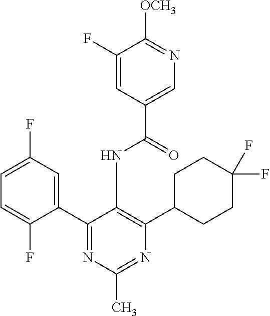 [SMILES: COc1ncc(C(=O)Nc2c(-c3cc(F)ccc3F)nc(C)nc2C2CCC(F)(F)CC2)cc1F]

In a similar manner as described in examples disclosed herein, utilizing the appropriately substituted starting materials and intermediates, the title compound was afforded as a colorless solid (0.00770 g, 43% yield): ¹H NMR (400 MHz, DMSO-d₆) δ 10.23 (s, 1H), 8.40 (d, J=2.0 Hz, 1H), 7.95 (dd, J=10.8, 2.0 Hz, 1H), 7.35-7.25 (m, 3H), 4.00 (s, 3H), 3.18-3.11 (m, 1H), 2.69 (s, 3H), 2.09-2.07 (m, 2H), 2.00-1.89 (m, 2H), 1.88-1.80 (m, 4H); MS (ES+) m/z 493.1 (M+1).

### Example 201

### Synthesis of N-(2-(4,4-difluorocyclohexyl)-4-(tetrahydro-2H-pyran-2-yl)pyridin-3-yl)-2-isopropylpyrimidine-5-carboxamide

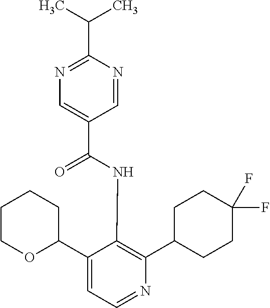 [SMILES: CC(C)c1ncc(C(=O)Nc2c(C3CCCCO3)ccnc2C2CCC(F)(F)CC2)cn1]

### Step 1. Preparation of tert-butyl (2-chloro-4-(3,4-dihydro-2H-pyran-6-yl)pyridin-3-yl)carbamate

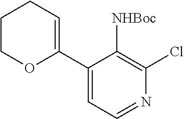 [SMILES: CC(C)(C)OC(=O)Nc1c(C2=CCCCO2)ccnc1Cl]

A mixture of tert-butyl N-(2-chloro-4-iodo-3-pyridyl)carbamate (0.500 g, 1.41 mmol), 2-(3,4-dihydro-2H-pyran-6-yl)-4,4,5,5-tetramethyl-1,3,2-dioxaborolane (0.355 g, 1.69 mmol), [1,1′-bis(diphenylphosphino) ferrocene]dichloropalladium(II) (0.103 g, 0.141 mmol) and potassium carbonate (0.584 g, 4.23 mmol) in dioxane (5 mL)/water(1 mL) was stirred at 70° C. for 12 h under a nitrogen atmosphere. The reaction mixture was cooled to ambient temperature and concentrated under reduced pressure. The residue was purified by flash silica gel chromatography, eluting with a gradient of 0-30% of ethyl acetate in petroleum ether, to afford the title compound as a colorless solid (0.400 g, 90% yield): ¹H NMR (400 MHz, CDCl₃) δ 8.19 (d, J=5.2 Hz, 1H) 7.27 (d, J=5.2 Hz, 1H), 6.63-6.37 (m, 1H), 5.32 (t, J=4.0 Hz, 1H), 4.22-4.15 (m, 2H), 2.24 (dt, J=4.4, 6.4 Hz, 2H), 2.01-1.87 (m, 2H), 1.51 (s, 9H).

### Step 2. Preparation of tert-butyl (2-(4,4-difluorocyclohex-1-en-1-yl)-4-(3,4-dihydro-2H-pyran-6-yl)pyridin-3-yl)carbamate

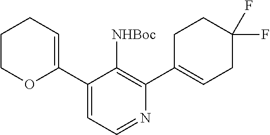 [SMILES: CC(C)(C)OC(=O)Nc1c(C2=CCCCO2)ccnc1C1=CCC(F)(F)CC1]

A mixture of 2-(4,4-difluorocyclohexen-1-yl)-4,4,5,5-tetramethyl-1,3,2-dioxaborolane (0.188 g, 0.772 mmol), tert-butyl (2-chloro-4-(3,4-dihydro-2H-pyran-6-yl)pyridin-3-yl) carbamate (0.200 g, 0.643 mmol), potassium carbonate (0.267 g, 1.93 mmol) and [1,1′-bis(diphenylphosphino)ferrocene]dichloropalladium(II) (0.0471 g, 0.0644 umol) in dioxane (2 mL)/water(0.4 mL) was stirred at 100° C. for 12 h under a nitrogen atmosphere. The reaction mixture was cooled to ambient temperature and concentrated under reduced pressure. The residue was purified by flash silica gel chromatography, eluting with a gradient of 0-32% of ethyl acetate in petroleum ether, to afford the title compound as a colorless solid (0.200 g, 79% yield): ¹H NMR (400 MHz, CDCl₃) δ 8.36 (d, J=4.8 Hz, 1H), 7.14 (d, J=4.8 Hz, 1H), 6.94 (s, 1H), 5.82 (s, 1H), 5.22 (t, J=4.0 Hz, 1H), 4.24-4.17 (m, 2H), 2.84 (s, 2H), 2.67 (t, J=14.4 Hz, 2H), 2.29-2.10 (m, 4H), 2.03-1.91 (m, 2H), 1.47 (s, 9H).

### Step 3. Preparation of tert-butyl (2-(4,4-difluorocyclohexyl)-4-(tetrahydro-2H-pyran-2-yl)pyridin-3-yl)carbamate

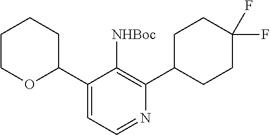 [SMILES: CC(C)(C)OC(=O)Nc1c(C2CCCCO2)ccnc1C1CCC(F)(F)CC1]

To a solution of tert-butyl (2-(4,4-difluorocyclohex-1-en-1-yl)-4-(3,4-dihydro-2H-pyran-6-yl)pyridin-3-yl) carbamate (0.200 g, 0.509 mmol) in methanol (10 mL) was added palladium on carbon (0.200 g, 10 wt %) under a nitrogen atmosphere. The suspension was degassed under vacuum and purged with hydrogen 3 times. The mixture was stirred under hydrogen (15 psi, balloon) at 25° C. for 12 h. The reaction mixture was filtered over Celite. The filtrate was concentrated under reduced pressure to give the title compound as a colorless solid (0.180 g, crude): ¹H NMR (400 MHz, CDCl₃) δ 8.47 (d, J=4.8 Hz, 1H), 7.18-7.17 (m, 1H), 6.94-6.73 (m, 1H), 4.42 (d, J=7.2 Hz, 1H), 4.16 (d, J=10.8 Hz, 1H), 3.69-3.56 (m, 1H), 3.13-2.91 (m, 1H), 2.31-2.17 (m, 2H), 2.08-1.65 (m, 12H), 1.54 (s, 9H).

### Step 4. Preparation of 2-(4,4-difluorocyclohexyl)-4-(tetrahydro-2H-pyran-2-yl)pyridin-3-amine

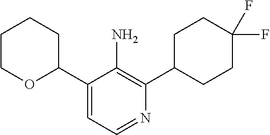 [SMILES: Nc1c(C2CCCCO2)ccnc1C1CCC(F)(F)CC1]

A mixture of tert-butyl (2-(4,4-difluorocyclohexyl)-4-(tetrahydro-2H-pyran-2-yl)pyridin-3-yl)carbamate (0.180 g, 0.454 mmol) in hydrogen chloride/dioxane (4 M, 5 mL) was stirred at 10° C. for 12 h. The reaction mixture was concentrated under reduced pressure to afford the title compound as a colorless solid (0.150 g, crude): ¹H NMR (400 MHz, DMSO-d₆) δ 15.15-14.37 (m, 1H), 7.93 (d, J=5.6 Hz, 1H), 7.58 (d, J=5.2 Hz, 1H), 6.57-6.02 (m, 2H), 4.66 (d, J=10.4 Hz, 1H), 4.09 (d, J=11.2 Hz, 1H), 3.65-3.57 (m, 2H), 2.27-2.01 (m, 3H), 2.00-1.72 (m, 8H), 1.66-1.56 (s, 2H).

### Step 5. Preparation of N-(2-(4,4-difluorocyclohexyl)-4-(tetrahydro-2H-pyran-2-yl)pyridin-3-yl)-2-isopropylpyrimidine-5-carboxamide

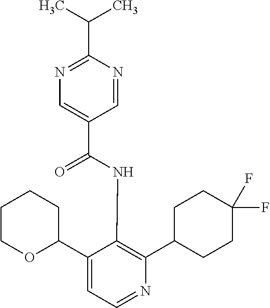 [SMILES: CC(C)c1ncc(C(=O)Nc2c(C3CCCCO3)ccnc2C2CCC(F)(F)CC2)cn1]

In a similar manner as described in examples disclosed herein, utilizing the appropriately substituted starting materials and intermediates, the title compound was afforded as a colorless solid (0.0327 g, 47% yield): ¹H NMR (400 MHz, DMSO-d₆) δ 10.50-10.10 (m, 1H), 9.25 (s, 2H), 8.51 (d, J=5.2 Hz, 1H), 7.36 (d, J=5.2 Hz, 1H), 4.70-4.34 (m, 1H), 4.01 (d, J=12.0 Hz, 1H), 3.59-3.42 (m, 1H), 3.28-3.19 (m, 1H), 3.09-2.98 (m, 1H), 2.12-1.66 (m, 10H), 1.59-1.45 (m, 3H), 1.34 (d, J=7.2 Hz, 6H), 1.30-1.21 (m, 1H); MS (ES+) m/z 445.2 (M+1).

In a similar manner as described in examples disclosed herein, utilizing the appropriately substituted starting materials and intermediates, the following compounds were prepared:

| Example | Structure | | MS (ES+) | |
|---|---|---|---|---|
| No. | Name | Yield | m/z | ¹H-NMR |
| 202 | 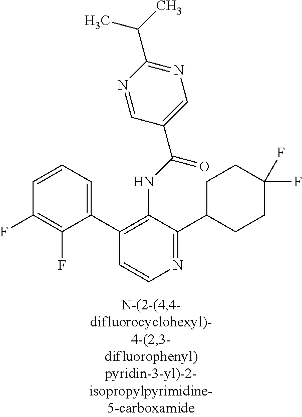 [SMILES: CC(C)c1ncc(C(=O)Nc2c(-c3cccc(F)c3F)ccnc2C2CCC(F)(F)CC2)cn1] | 53% | 473.3 (M + 1) | ¹H NMR (400 MHz, DMSO-d₆) δ 10.34 (s, 1H), 8.95 (s, 2H), 8.63 (d, J = 4.8 Hz, 1H), 7.49-7.36 (m, 2H), 7.30-7.20 (m, 1H), 7.19-7.11 (m, 1H), 3.19 (dt, J = 14.0, 6.8 Hz, 2H), 2.15- 1.74 (m, 8H), 1.28 (d, J = 6.8 Hz, 6H) |

### Example 203

### Synthesis of N-(2-(4,4-difluorocyclohexyl)-4-(tetrahydro-2H-pyran-2-yl)pyridin-3-yl)-2-isopropylpyrimidine-5-carboxamide

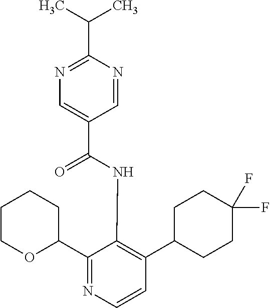 [SMILES: CC(C)c1ncc(C(=O)Nc2c(C3CCC(F)(F)CC3)ccnc2C2CCCCO2)cn1]

### Step 1. Preparation of N-(4-(4,4-difluorocyclohex-1-en-1-yl)-2-(3,4-dihydro-2H-pyran-6-yl)pyridin-3-yl)-2-isopropylpyrimidine-5-carboxamide

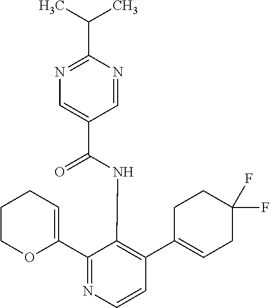 [SMILES: CC(C)c1ncc(C(=O)Nc2c(C3=CCC(F)(F)CC3)ccnc2C2=CCCCO2)cn1]

To a mixture of 2-(3,4-dihydro-2H-pyran-6-yl)-4,4,5,5-tetramethyl-1,3,2-dioxaborolane (0.0641 g, 0.305 mmol) and N-(2-chloro-4-(4,4-difluorocyclohex-1-en-1-yl)pyridin-3-yl)-2-isopropylpyrimidine-5-carboxamide (0.100 g, 0.255 mmol) in dioxane (3 mL) and water (0.6 mL) were added [1,1′-bis(diphenylphosphino)ferrocene]dichloropalladium(II) (0.0186 g, 0.0255 mmol) and potassium carbonate (0.106 g, 0.764 mmol). The mixture was degassed and purged with nitrogen 3 times. The reaction mixture was stirred at 100° C. for 12 h under nitrogen atmosphere. The reaction mixture was cooled to ambient temperature and concentrated under reduced pressure. The residue was purified by flash silica gel chromatography, eluting with a gradient of 0-100% of ethyl acetate in petroleum ether, to afford the title compound as a yellow solid (0.0300 g, 25% yield: ¹H NMR (400 MHz, DMSO-d₆) δ 10.13 (s, 1H), 9.11 (s, 2H), 8.46 (d, J=4.8 Hz, 1H), 7.29 (d, J=4.8 Hz, 1H), 5.60 (s, 1H), 5.39-5.35 (m, 1H), 3.98-3.94 (m, 2H), 3.26-3.21 (m, 1H), 2.36-2.29 (m, 4H), 2.18-2.01 (m, 4H), 1.82-1.74 (m, 2H), 1.33 (d, J=6.8 Hz, 6H).

### Step 2. Preparation of N-(2-(4,4-difluorocyclohexyl)-4-(tetrahydro-2H-pyran-2-yl)pyridin-3-yl)-2-isopropylpyrimidine-5-carboxamide

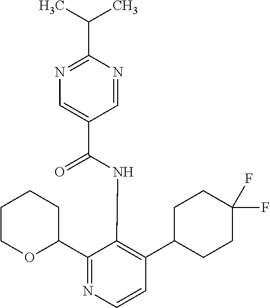 [SMILES: CC(C)c1ncc(C(=O)Nc2c(C3CCC(F)(F)CC3)ccnc2C2CCCCO2)cn1]

To a solution of N-(4-(4,4-difluorocyclohex-1-en-1-yl)-2-(3,4-dihydro-2H-pyran-6-yl)pyridin-3-yl)-2-isopropylpyrimidine-5-carboxamide (0.0200 g, 0.0454 mmol) in methanol (3 mL) was added palladium on activated carbon (0.0200 g, 0.0187 mmol, 10 wt %) under a nitrogen atmosphere. The suspension was degassed under vacuum and purged with hydrogen several times. The mixture was stirred under hydrogen (15 psi, balloon) at 10° C. for 12 h. The reaction mixture was filtered over Celite. The filtrate was concentrated under reduced pressure. The residue was purified by preparative HPLC, eluting with a gradient of 32-67% of acetonitrile in water containing 0.225% formic acid, to afford the title compound as a colorless solid (0.00920 g, 45% yield): ¹H NMR (400 MHz, CDCl₃) δ 9.56 (s, 1H), 9.26 (s, 2H), 8.41 (d, J=5.2 Hz, 1H), 7.25 (d, J=5.2 Hz, 1H), 4.65 (dd, J=11.2, 2.0 Hz, 1H), 4.29-4.17 (m, 1H), 3.73-3.59 (m, 1H), 3.37 (septet, J=6.8 Hz, 1H), 2.82-2.62 (m, 1H), 2.29-2.09 (m, 3H), 2.02-1.87 (m, 3H), 1.85-1.56 (m, 8H), 1.43 (d, J=6.8 Hz, 6H); MS (ES+) m/z 445.3 (M+1).

### Example 204

### Synthesis of N-(4-(4,4-difluorocyclohexyl)-2-(2,5-difluorophenyl)pyridin-3-yl)-2-isopropoxypyrimidine-5-carboxamide

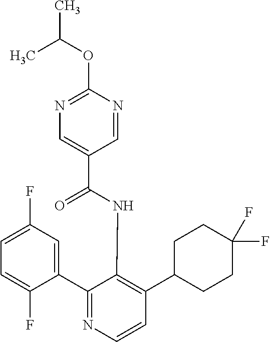 [SMILES: CC(C)Oc1ncc(C(=O)Nc2c(C3CCC(F)(F)CC3)ccnc2-c2cc(F)ccc2F)cn1]

### Step 1. Preparation of 4-(4,4-difluorocyclohex-1-en-1-yl)-2-(2,5-difluorophenyl)-3-nitropyridine

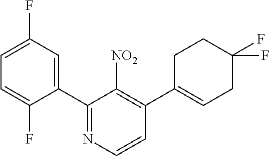 [SMILES: O=[N+]([O-])c1c(C2=CCC(F)(F)CC2)ccnc1-c1cc(F)ccc1F]

To a solution of 4-chloro-2-(2,5-difluorophenyl)-3-nitropyridine (1.00 g, 3.70 mmol), 2-(4,4-difluorocyclohex-1-en-1-yl)-4,4,5,5-tetramethyl-1,3,2-dioxaborolane (1.10 g, 4.51 mmol) and potassium carbonate (1.53 g, 11.0 mmol) in dioxane (12 mL)/water (3 mL) under a nitrogen atmosphere was added [1,1′-bis(diphenylphosphino)ferrocene]dichloropalladium(II) (0.270 g, 0.369 mmol). The mixture was stirred at 80° C. for 12 h. The mixture was cooled to 25° C. and concentrated under reduced pressure. The residue was purified by flash silica gel chromatography, eluting with a gradient of 35-50% of ethyl acetate in petroleum ether, to afford the title compound as a yellow oil (1.20 g, 82% yield): ¹H NMR (400 MHz, DMSO-d₆) δ 8.90 (d, J=5.2 Hz, 1H), 7.72 (d, J=5.2 Hz, 1H), 7.52-7.37 (m, 3H), 5.70 (s, 1H), 2.75-2.64 (m, 2H), 2.61-2.54 (m, 2H), 2.17 (tt, J=13.9, 6.8 Hz, 2H); MS (ES+) m/z 353.1 (M+1).

### Step 2. Preparation of 4-(4,4-difluorocyclohexyl)-2-(2,5-difluorophenyl)pyridin-3-amine

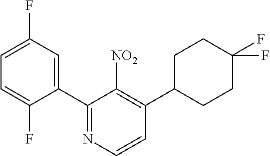 [SMILES: O=[N+]([O-])c1c(C2CCC(F)(F)CC2)ccnc1-c1cc(F)ccc1F]

To a solution of 4-(4,4-difluorocyclohex-1-en-1-yl)-2-(2,5-difluorophenyl)-3-nitropyridine (1.20 g, 3.41 mmol) in methanol (15 mL) was added palladium on carbon (1.20 g, 10 wt %) under a nitrogen atmosphere. The suspension was degassed and purged with hydrogen 3 times. The mixture was stirred under hydrogen (15 psi, balloon) at 25° C. for 12 h. The resulting mixture was filtered over Celite. The filter cake was washed with methanol (30 mL). The filtrate was concentrated under reduced pressure to afford the title compound as a colorless solid (0.900 g, 73% yield): ¹H NMR (400 MHz, DMSO-d₆) δ 7.84 (d, J=4.8 Hz, 1H), 7.38-7.27 (m, 2H), 7.23 (ddd, J=8.8, 5.6, 3.2 Hz, 1H), 7.06 (d, J=4.8 Hz, 1H), 4.89 (s, 2H), 2.89 (t, J=11.2 Hz, 1H), 2.20-1.85 (m, 6H), 1.66-1.52 (m, 2H); MS (ES+) m/z 325.1 (M+1).

### Step 3. Preparation of 2-chloro-N-(4-(4,4-difluorocyclohexyl)-2-(2,5-difluorophenyl)pyridin-3-yl)pyrimidine-5-carboxamide

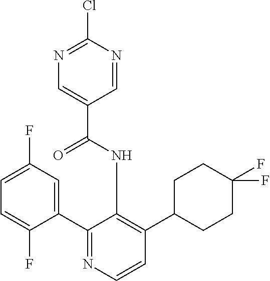 [SMILES: O=C(Nc1c(C2CCC(F)(F)CC2)ccnc1-c1cc(F)ccc1F)c1cnc(Cl)nc1]

In a similar manner as described in examples disclosed herein, utilizing the appropriately substituted starting materials and intermediates, the title compound was afforded as a yellow solid (0.240 g, 37% yield): ¹H NMR (400 MHz, DMSO-d₆) δ 10.49 (s, 1H), 8.98 (s, 2H), 8.61 (d, J=5.2 Hz, 1H), 7.57 (d, J=5.2 Hz, 1H), 7.34-7.26 (m, 2H), 7.25-7.19 (m, 1H), 3.09-2.96 (m, 1H), 2.21-2.01 (m, 3H), 1.93-1.80 (m, 3H), 1.78-1.65 (m, 2H); MS (ES+) m/z 465.1, 467.1 (M+1).

### Step 4. Preparation of N-(4-(4,4-difluorocyclohexyl)-2-(2,5-difluorophenyl)pyridin-3-yl)-2-isopropoxypyrimidine-5-carboxamide

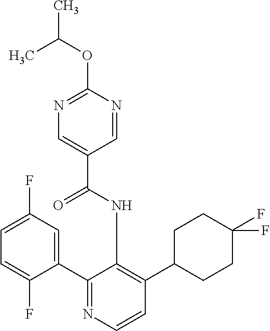 [SMILES: CC(C)Oc1ncc(C(=O)Nc2c(C3CCC(F)(F)CC3)ccnc2-c2cc(F)ccc2F)cn1]

A solution of 2-chloro-N-(4-(4,4-difluorocyclohexyl)-2-(2,5-difluorophenyl)pyridin-3-yl)pyrimidine-5-carboxamide (0.0500 g, 0.107 mmol) and cesium carbonate (0.105 g, 0.322 mmol) in isopropanol (1 mL) was stirred at 80° C. for 12 h. The mixture was cooled to 25° C. and concentrated under reduced pressure. The residue was purified by preparative HPLC, eluting with a gradient of 40-70% of acetonitrile in water containing 0.225% formic acid, to afford the title compound as a colorless solid (0.0108 g, 20% yield): ¹H NMR (400 MHz, MeOD) δ8.85 (s, 2H), 8.62 (d, J=5.2 Hz, 1H), 7.63 (d, J=5.2 Hz, 1H), 7.28-7.02 (m, 3H), 5.40-5.35 (m, 1H), 3.11-2.96 (m, 1H), 2.24-2.11 (m, 2H), 2.00-1.80 (m, 6H), 1.40 (d, J=6.0 Hz, 6H); MS (ES+) m/z 489.2 (M+1).

### Example 205

### Synthesis of 6-(1-cyclopropylethoxy)-N-(4-(4,4-difluorocyclohexyl)-2-(2,5-difluorophenyl)pyridin-3-yl)-5-fluoronicotinamide

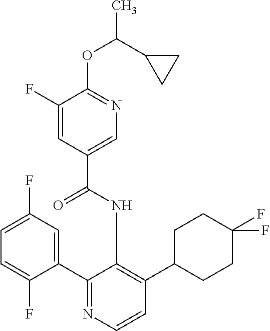 [SMILES: CC(Oc1ncc(C(=O)Nc2c(C3CCC(F)(F)CC3)ccnc2-c2cc(F)ccc2F)cc1F)C1CC1]

### Step 1. Preparation of N-(4-(4,4-difluorocyclohexyl)-2-(2,5-difluorophenyl)pyridin-3-yl)-5,6-difluoronicotinamide

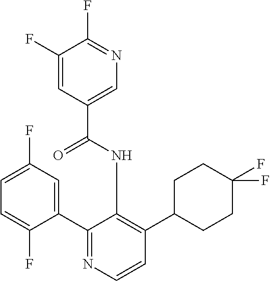 [SMILES: O=C(Nc1c(C2CCC(F)(F)CC2)ccnc1-c1cc(F)ccc1F)c1cnc(F)c(F)c1]

In a similar manner as described in examples disclosed herein, utilizing the appropriately substituted starting materials and intermediates, the title compound was afforded as a yellow solid (0.180 g, 56% yield): ¹H NMR (400 MHz, DMSO-d₆) δ 10.36 (s, 1H), 8.61 (d, J=5.2 Hz, 1H), 8.36 (s, 1H), 8.26 (td, J=9.6, 1.6 Hz, 1H), 7.56 (d, J=5.2 Hz, 1H), 7.36-7.29 (m, 1H), 7.29-7.25 (m, 1H), 7.23-7.19 (m, 1H), 3.00 (t, J=12.0 Hz, 1H), 2.16-2.04 (m, 2H), 1.94-1.59 (m, 6H); MS (ES+) m/z 466.1 (M+1).

### Step 2. Preparation of 6-(1-cyclopropylethoxy)-N-(4-(4,4-difluorocyclohexyl)-2-(2,5-difluorophenyl)pyridin-3-yl)-5-fluoronicotinamide

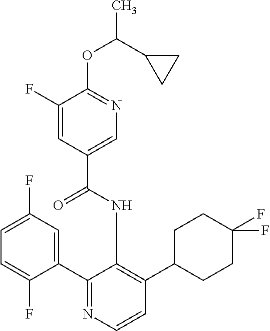 [SMILES: CC(Oc1ncc(C(=O)Nc2c(C3CCC(F)(F)CC3)ccnc2-c2cc(F)ccc2F)cc1F)C1CC1]

To a solution of N-(4-(4,4-difluorocyclohexyl)-2-(2,5-difluorophenyl)pyridin-3-yl)-5,6-difluoronicotinamide (0.0500 g, 0.107 mmol) and 1-cyclopropylethan-1-ol (0.0180 g, 0.208 mmol) in tetrahydrofuran (2 mL) at 0° C. was added potassium tert-butoxide (0.2 mL, 1 M in tetrahydrofuran). The mixture was stirred at 25° C. for 12 h. The reaction mixture was diluted with water (5 mL) and extracted with ethyl acetate (2×7 mL). The combined organic layers were washed with brine (8 mL), dried over sodium sulfate, filtered, and concentrated under reduced pressure. The residue was purified by preparative HPLC, eluting with a gradient of 54-82% of acetonitrile in water containing 0.225% formic acid, to afford the title compound as a colorless solid (0.0354 g, 61% yield): ¹H NMR (400 MHz, DMSO-d₆) δ 10.09 (s, 1H), 8.58 (d, J=5.2 Hz, 1H), 8.31 (d, J=2.0 Hz, 1H), 7.89 (dd, J=2.0, 11.2 Hz, 1H), 7.54 (d, J=5.2 Hz, 1H), 7.33-7.13 (m, 3H), 4.83-4.73 (m, 1H), 2.99 (t, J=12.0 Hz, 1H), 2.17-2.04 (m, 2H), 1.85 (d, J=11.2 Hz, 4H), 1.79-1.60 (m, 2H), 1.37 (d, J=6.0 Hz, 3H), 1.22-1.14 (m, 1H), 0.52 (t, J=9.2 Hz, 2H), 0.37 (d, J=4.8 Hz, 2H); MS (ES+) m/z 532.2 (M+1).

### Example 206

### Synthesis of N-(4-(4,4-difluorocyclohexyl)-2-phenylpyridin-3-yl)-2-isopropylpyrimidine-5-carboxamide

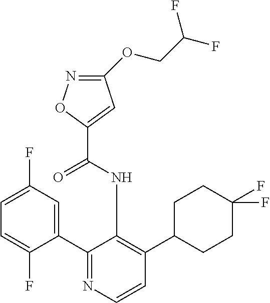 [SMILES: O=C(Nc1c(C2CCC(F)(F)CC2)ccnc1-c1cc(F)ccc1F)c1cc(OCC(F)F)no1]

In a similar manner as described in examples disclosed herein, utilizing the appropriately substituted starting materials and intermediates, the title compound was afforded as a colorless solid (0.0300 g, 23% yield): ¹H NMR (400 MHz, CDCl₃) δ 8.69 (d, J=5.2 Hz, 1H), 8.07 (d, J=4.4 Hz, 1H), 7.40 (d, J=5.2 Hz, 1H), 7.32-7.27 (m, 1H), 7.15-7.05 (m, 2H), 6.59 (s, 1H), 6.13 (tt, J=54, 4.0 Hz, 1H), 4.50 (td, J=13.2, 4.4 Hz, 2H), 2.86-2.75 (m, 1H), 2.31-2.17 (m, 2H), 2.01 (d, J=7.2 Hz, 2H), 1.88-1.74 (m, 4H); MS (ES+) m/z 500.4 (M+1).

### Example 207

### Synthesis of N-(4-(4,4-difluorocyclohexyl)-2-phenylpyridin-3-yl)-2-isopropylpyrimidine-5-carboxamide

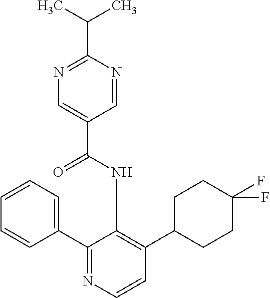 [SMILES: CC(C)c1ncc(C(=O)Nc2c(C3CCC(F)(F)CC3)ccnc2-c2ccccc2)cn1]

### Step 1. Preparation of tert-butyl (2-chloro-4-(4,4-difluorocyclohex-1-en-1-yl)pyridin-3-yl)carbamate

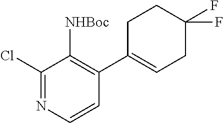 [SMILES: CC(C)(C)OC(=O)Nc1c(C2=CCC(F)(F)CC2)ccnc1Cl]

To a mixture of tert-butyl (2-chloro-4-iodopyridin-3-yl)carbamate (1.00 g, 2.82 mmol), 2-(4,4-difluorocyclohex-1-en-1-yl)-4,4,5,5-tetramethyl-1,3,2-dioxaborolane (0.771 g, 3.16 mmol) and potassium carbonate (1.17 g, 8.46 mmol) in 1,4-dioxane (10 mL) and water (2 mL) was added [1,1′-bis(diphenylphosphino)ferrocene]dichloropalladium(II) (0.206 g, 0.282 mmol) under nitrogen atmosphere. The mixture was stirred at 90° C. for 12 h. After being cooled to ambient temperature, the reaction mixture was concentrated under reduced pressure. The residue was poured into water (20 mL) and extracted with ethyl acetate (3×20 mL). The combined organic extracts were dried over anhydrous sodium sulfate, filtered and the filtrate was concentrated in vacuo. The residue was purified by flash silica gel chromatography, eluting with 50% of ethyl acetate in petroleum ether, to afford the title compound as a yellow solid (0.800 g, 82% yield): ¹H NMR (400 MHz, CDCl₃) 98.19 (d, J=5.2 Hz, 1H), 7.07 (d, J=5.2 Hz, 1H), 6.25 (s, 1H), 5.67 (s, 1H), 2.74-2.56 (m, 4H), 2.15 (tt, J=6.6, 13.6 Hz, 2H), 1.47 (s, 9H).

### Step 2. Preparation of 2-chloro-4-(4,4-difluorocyclohex-1-en-1-yl)pyridin-3-amine

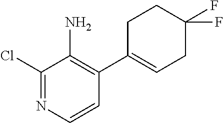 [SMILES: Nc1c(C2=CCC(F)(F)CC2)ccnc1Cl]

A mixture of tert-butyl (2-chloro-4-(4,4-difluorocyclohex-1-en-1-yl)pyridin-3-yl)carbamate (0.800 g, 2.32 mmol) in hydrochloric acid in methanol (3 mL, 4 M) was stirred at 25° C. for 12 h. The reaction mixture was concentrated under reduced pressure. The residue was poured into water (10 mL) and adjusted to pH=8 with aqueous sodium hydroxide (5 mL, 2 M). The mixture was extracted with ethyl acetate (3×20 mL). The combined organic layers were dried over anhydrous sodium sulfate, filtered and concentrated in vacuo to afford the title compound as a yellow oil (0.550 g, 97% yield): ¹H NMR (400 MHz, DMSO-d₆) δ 7.57 (d, J=4.4 Hz, 1H), 6.92 (d, J=4.4 Hz, 1H), 5.66 (s, 1H), 5.21 (s, 2H), 2.77-2.61 (m, 2H), 2.46-2.39 (m, 2H), 2.29-2.13 (m, 2H).

### Step 3. Preparation of N-(2-chloro-4-(4,4-difluorocyclohex-1-en-1-yl)pyridin-3-yl)-2-isopropylpyrimidine-5-carboxamide

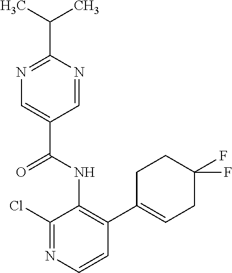 [SMILES: CC(C)c1ncc(C(=O)Nc2c(C3=CCC(F)(F)CC3)ccnc2Cl)cn1]

To a mixture of 2-chloro-4-(4,4-difluorocyclohex-1-en-1-yl)pyridin-3-amine (0.100 g, 0.355 mmol), 2-isopropylpyrimidine-5-carboxylic acid (0.0679 g, 0.408 mmol) and 2-chloro-1-methyl-pyridin-1-ium iodide (0.136 g, 0.533 mmol) in tetrahydrofuran (2 mL) was added N,N-diisopropylethylamine (0.183 g, 1.42 mmol,). The mixture was stirred at 70° C. for 12 h and then aqueous sodium hydroxide (2 mL, 2 M) was added. The mixture was stirred at 25° C. for 12 h. The reaction mixture was concentrated under reduced pressure. The residue was poured into water (20 mL) and extracted with ethyl acetate (3×15 mL). The combined organic extracts were dried over anhydrous sodium sulfate, filtered and the filtrate was concentrated in vacuo. The residue was purified by flash silica gel chromatography, eluting with a gradient of 50% of ethyl acetate in petroleum ether, to afford the title compound as a yellow solid (0.100 g, 72% yield): ¹H NMR (400 MHz, DMSO-d₆) δ 10.54 (s, 1H), 9.16 (s, 2H), 8.37 (d, J=4.4 Hz, 1H), 7.43 (d, J=4.4 Hz, 1H), 5.69 (s, 1H), 3.31-3.18 (m, 1H), 2.62 (t, J=14.6 Hz, 2H), 2.56-2.51 (m, 2H), 2.18-2.02 (m, 2H), 1.32 (d, J=6.8 Hz, 6H).

### Step 4. Preparation of N-(4-(4,4-difluorocyclohex-1-en-1-yl)-2-phenylpyridin-3-yl)-2-isopropylpyrimidine-5-carboxamide

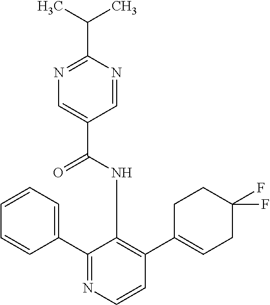 [SMILES: CC(C)c1ncc(C(=O)Nc2c(C3=CCC(F)(F)CC3)ccnc2-c2ccccc2)cn1]

To a mixture of N-(2-chloro-4-(4,4-difluorocyclohex-1-en-1-yl)pyridin-3-yl)-2-isopropylpyrimidine-5-carboxamide (0.100 g, 0.254 mmol), phenylboronic acid (0.0465 g, 0.381 mmol) and potassium carbonate (0.105 g, 0.763 mmol) in dioxane (1.5 mL) and water (0.3 mL) was added [1,1′-bis(diphenylphosphino)ferrocene]dichloropalladium(II) (0.0186 g, 0.0254 mmol) under a nitrogen atmosphere. The mixture was stirred at 90° C. for 12 h. After being cooled to ambient temperature, the reaction mixture was concentrated under reduced pressure. The residue was purified by flash silica gel chromatography, eluting with a gradient of 20% of ethyl acetate in petroleum ether, to afford the title compound as a colorless solid (0.0800 g, 72% yield); ¹H NMR (400 MHz, CDCl₃) δ 8.84 (s, 2H), 8.57 (d, J=4.8 Hz, 1H), 7.51 (s, 2H), 7.46-7.37 (m, 4H), 7.19 (d, J=4.8 Hz, 1H), 5.61 (s, 1H), 3.27 (td, J=6.8, 13.6 Hz, 1H), 2.78-2.48 (m, 4H), 2.14 (tt, J=6.4, 13.6 Hz, 2H), 1.36 (d, J=6.8 Hz, 6H).

### Step 5. Preparation of N-(4-(4,4-difluorocyclohexyl)-2-phenylpyridin-3-yl)-2-isopropylpyrimidine-5-carboxamide

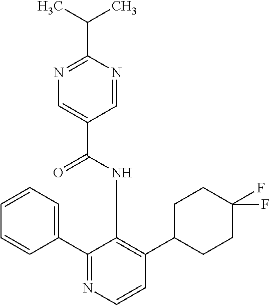 [SMILES: CC(C)c1ncc(C(=O)Nc2c(C3CCC(F)(F)CC3)ccnc2-c2ccccc2)cn1]

To a mixture of N-(4-(4,4-difluorocyclohex-1-en-1-yl)-2-phenylpyridin-3-yl)-2-isopropylpyrimidine-5-carboxamide (0.0800 g, 0.184 mmol) in methanol (3 mL) was added palladium on carbon (0.0800 g, 10 wt %) under nitrogen atmosphere. The mixture was stirred at 25° C. under hydrogen (15 Psi) atmosphere for 12 h. The reaction mixture was filtered over Celite. The residue was purified by preparative HPLC, eluting with a gradient of 34-64% of acetonitrile in water containing 0.225% formic acid, to afford the title compound as a colorless solid (0.00620 g, 8% yield): ¹H NMR (400 MHz, CDCl₃) δ 8.92 (s, 2H), 8.61 (d, J=4.4 Hz, 1H), 7.50 (d, J=4.0 Hz, 2H), 7.42 (d, J=3.2 Hz, 3H), 7.34 (d, J=4.8 Hz, 1H), 3.30 (dt, J=13.8, 6.8 Hz, 1H), 2.88-2.73 (m, 1H), 2.33-2.18 (m, 2H), 2.14-1.97 (m, 2H), 1.89-1.73 (m, 4H), 1.38 (d, J=6.8 Hz, 6H); MS (ES+) m/z 437.3 (M+1).

### Example 208

In a similar manner as described in Example 207, utilizing the appropriately substituted starting materials and intermediates, the following compounds were prepared:

| Example | | | MS (ES+) | |
|---|---|---|---|---|
| No. | Structure | Yield | m/z | ¹H-NMR |
| 208 | 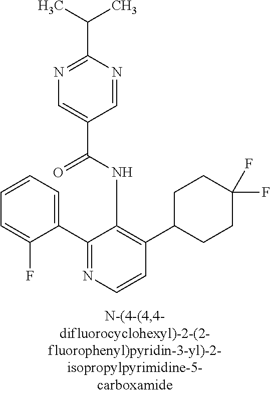 [SMILES: CC(C)c1ncc(C(=O)Nc2c(C3CCC(F)(F)CC3)ccnc2-c2ccccc2F)cn1] | 24% | 455.3 (M + 1) | ¹H NMR (400 MHz,CDCl₃) δ 8.94 (s, 2H), 8.69 (d, J = 4.8 Hz, 1H), 7.72 (d, J = 4.8 Hz, 1H), 7.59 (t, J = 7.2 Hz, 1H), 7.45-7.36 (m, 2H), 7.31-7.28 (m, 1H), 7.21-7.14 (m, 1H), 3.30 (dt, J = 13.6, 6.8 Hz, 1H), 2.92- 2.76 (m, 1H), 2.26 (d, J = 2.8 Hz, 2H), 2.05 (d, J = 9.6 Hz, 2H), 1.93- 1.69 (m,4H), 1.44-1.34 (m, 6H) |
| 209 | 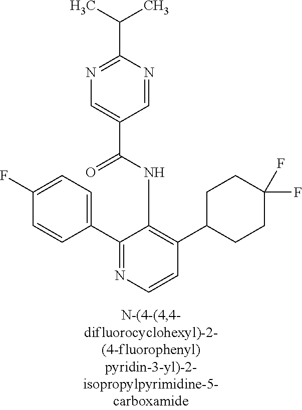 [SMILES: CC(C)c1ncc(C(=O)Nc2c(C3CCC(F)(F)CC3)ccnc2-c2ccc(F)cc2)cn1] | 63% | 455.2 (M + 1) | ¹H NMR (400 MHz, DMSO-d₆) δ 10.36 (s, 1H), 9.03 (s, 2H), 8.58 (d, J = 4.8 Hz, 1H), 7.62 (dd, J = 8.8, 5.6 Hz, 2H), 7.47 (d, J = 5.2 Hz, 1H), 7.23 (t, J = 8.8 Hz, 2H), 3.21 (dt, J = 13.6, 6.8 Hz, 1H), 3.07-2.93 (m, 1H), 2.11-1.83 (m, 6H), 1.76-1.65 (m, 2H), 1.30 (d, J = 6.8 Hz, 6H) |

### Example 210

### Synthesis of N-(2-(4,4-difluorocyclohexyl)-4-(2,5-difluorophenyl)pyridin-3-yl)-6-isopropoxypyridazine-4-carboxamide

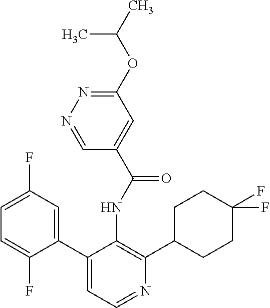 [SMILES: CC(C)Oc1cc(C(=O)Nc2c(-c3cc(F)ccc3F)ccnc2C2CCC(F)(F)CC2)cnn1]

### Step 1. Preparation of 6-chloro-N-(2-(4,4-difluorocyclohexyl)-4-(2,5-difluorophenyl)pyridin-3-yl)pyridazine-4-carboxamide

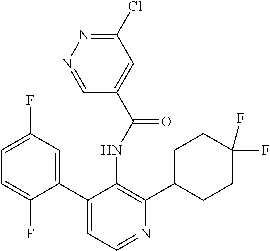 [SMILES: O=C(Nc1c(-c2cc(F)ccc2F)ccnc1C1CCC(F)(F)CC1)c1cnnc(Cl)c1]

In a similar manner as described in examples disclosed herein, utilizing the appropriately substituted starting materials and intermediates, the title compound was afforded as a colorless solid (0.0700 g, 32%): ¹H NMR (400 MHz, DMSO-d₆) δ 10.65 (s, 1H), 9.36 (d, J=1.6 Hz, 1H), 8.65 (d, J=4.8 Hz, 1H), 8.08 (d, J=1.6 Hz, 1H), 7.40-7.20 (m, 4H), 3.22-3.16 (m, 1H), 2.15-2.01 (m, 3H), 1.96-1.75 (m, 5H).

### Step 2. Preparation of N-(2-(4,4-difluorocyclohexyl)-4-(2,5-difluorophenyl)pyridin-3-yl)-6-isopropoxypyridazine-4-carboxamide

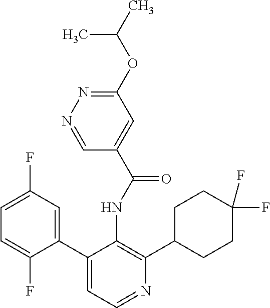 [SMILES: CC(C)Oc1cc(C(=O)Nc2c(-c3cc(F)ccc3F)ccnc2C2CCC(F)(F)CC2)cnn1]

To a mixture of 6-chloro-N-(2-(4,4-difluorocyclohexyl)-4-(2,5-difluorophenyl)pyridin-3-yl)pyridazine-4-carboxamide (0.0600 g, 0.129 mmol) in isopropanol (1 mL) was added cesium carbonate (0.126 g, 0.387 mmol). The resulting mixture was stirred at 90° C. for 36 h under a nitrogen atmosphere. The mixture was diluted with N,N-dimethylfomamide (1 mL) and ethyl acetate (10 mL). The residue was purified by preparative HPLC, eluting with a gradient of 50-75% of acetonitrile in water containing 0.225% formic acid, to afford the title compound as a colorless solid (0.0228 g, 35% yield): ¹H NMR (400 MHz, CDCl₃) δ 8.98 (s, 1H), 8.71 (d, J=4.8 Hz, 1H), 7.63 (d, J=1.6 Hz, 1H), 7.23 (d, J=4.8 Hz, 1H), 7.19-7.04 (m, 4H), 5.63 (td, J=6.4, 12.4 Hz, 1H), 3.02-2.90 (m, 1H), 2.33-2.10 (m, 4H), 1.99-1.90 (m, 2H), 1.87-1.71 (m, 2H), 1.44 (d, J=6.4 Hz, 6H); MS (ES+) m/z 489.2 (M+1).

### Example 211

### Synthesis of N-(2-((3,3-difluoroazetidin-1-yl)methyl)-4-(2,5-difluorophenyl)pyridin-3-yl)-2-isopropylpyrimidine-5-carboxamide

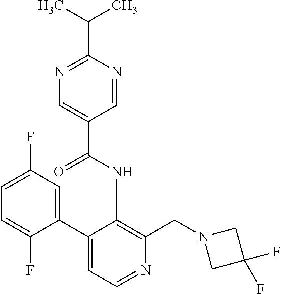 [SMILES: CC(C)c1ncc(C(=O)Nc2c(-c3cc(F)ccc3F)ccnc2CN2CC(F)(F)C2)cn1]

### Step 1. Preparation of 3-amino-4-(2,5-difluorophenyl) picolinaldehyde

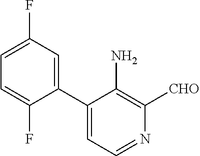 [SMILES: Nc1c(-c2cc(F)ccc2F)ccnc1C=O]

To a solution of 2-chloro-4-(2,5-difluorophenyl)-3-nitropyridine (1.00 g, 3.70 mmol) in dimethyl formamide (15 mL) was added [1,1′-bis(diphenylphosphino)ferrocene]dichloropalladium(II) (0.270 g, 0.369 mmol), triethylsilane (1.30 g, 11.1 mmol), and triethylamine (1.10 g, 10.8 mmol) at 25° C. under argon atmosphere. The suspension was degassed and purged with carbon monoxide three times. The mixture was stirred at 80° C. for 12 h under carbon monoxide (50 psi). The reaction mixture was cooled to ambient temperature, diluted with water (30 mL), and extracted with ethyl acetate (2×30 mL). The combined organic layers were washed with brine (30 mL), dried over sodium sulfate, filtered and concentrated under reduced pressure. The residue was purified by flash silica gel chromatography, eluting with a gradient of 35-50% of ethyl acetate in petroleum ether, to afford the title compound as a yellow solid (0.220 g, 17% yield): ¹H NMR (400 MHz, DMSO-d₆) δ 9.98 (s, 1H), 8.10 (d, J=4.4 Hz, 1H), 7.41-7.30 (m, 4H), 6.95 (s, 2H); MS (ES+) m/z 235.1 (M+1).

### Step 2. Preparation of 2-((3,3-difluoroazetidin-1-yl)methyl)-4-(2,5-difluorophenyl)pyridin-3-amine

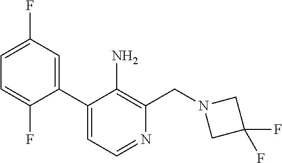 [SMILES: Nc1c(-c2cc(F)ccc2F)ccnc1CN1CC(F)(F)C1]

A solution of 3-amino-4-(2,5-difluorophenyl)picolinaldehyde (0.150 g, 0.429 mmol, 67% purity), 3,3-difluoroazetidine hydrochloride (0.0830 g, 0.640 mmol) and N,N-diisopropylethylamine (0.111 g, 0.858 mmol) in 1, 2-dichloroethane (5 mL) was stirred at 25° C. for 1 h. Sodium triacetoxy borohydride (0.455 g, 2.15 mmol) was added. The final mixture was stirred at 25° C. for 12 h. The reaction mixture was diluted with water (20 mL) and extracted with ethyl acetate (2×20 mL). The combined organic layers were washed with brine (15 mL), dried over sodium sulfate, filtered, and concentrated under reduced pressure. The residue was purified by preparative HPLC, eluting with a gradient of 10-40% of acetonitrile in water containing 0.225% formic acid, to afford the title compound as a yellow oil (0.100 g, 69% yield): ¹H NMR (400 MHz, MeOD) δ7.84 (d, J=5.2 Hz, 1H), 7.32-7.12 (m, 3H), 7.07 (d, J=5.2 Hz, 1H), 3.95 (s, 2H), 3.67 (t, J=12.0 Hz, 4H); MS (ES+) m/z 312.1 (M+1).

### Step 3. Preparation of N-(2-((3,3-difluoroazetidin-1-yl)methyl)-4-(2,5-difluorophenyl)pyridin-3-yl)-2-isopropylpyrimidine-5-carboxamide

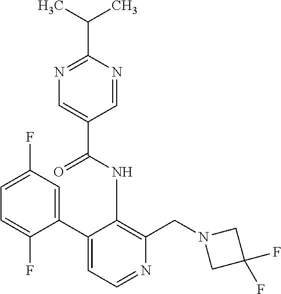 [SMILES: CC(C)c1ncc(C(=O)Nc2c(-c3cc(F)ccc3F)ccnc2CN2CC(F)(F)C2)cn1]

In a similar manner as described in examples disclosed herein, utilizing the appropriately substituted starting materials and intermediates, the title compound was afforded as a gray solid (0.00270 g, 6% yield): ¹H NMR (400 MHz, DMSO-d₆) δ 10.47-10.36 (m, 1H), 8.97 (s, 2H), 8.61 (d, J=4.8 Hz, 1H), 7.46 (d, J=4.8 Hz, 1H), 7.39-7.33 (m, 1H), 7.31-7.21 (m, 2H), 3.95 (s, 2H), 3.70 (t, J=12.4 Hz, 4H), 3.20 (td, J=6.8, 13.6 Hz, 1H), 1.29 (d, J=6.8 Hz, 6H); MS (ES+) m/z 460.3 (M+1).

### Example 212 and Example 213

### Synthesis of N-(2-(1-(3,3-difluoroazetidin-1-yl) ethyl)-4-(2,5-difluorophenyl)pyridin-3-yl)-2-isopropylpyrimidine-5-carboxamide formate salt P1 and P2/

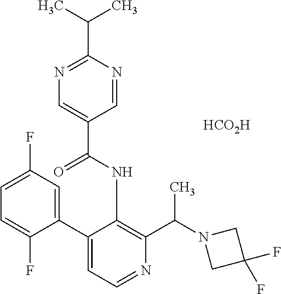 [SMILES: CC(C)c1ncc(C(=O)Nc2c(-c3cc(F)ccc3F)ccnc2C(C)N2CC(F)(F)C2)cn1 ; "HCO2H"]

### Step 1. Preparation of 4-(2,5-difluorophenyl)-2-(1-ethoxyvinyl)-3-nitropyridine

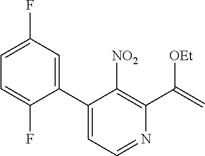 [SMILES: C=C(OCC)c1nccc(-c2cc(F)ccc2F)c1[N+](=O)[O-]]

To a solution of 2-chloro-4-(2,5-difluorophenyl)-3-nitropyridine (2.00 g, 7.39 mmol) in dimethyl formamide (100 mL) was added tributyl(1-ethoxyvinyl)stannane (5.13 g, 14.2 mmol) and [1,1′-bis(diphenylphosphino) ferrocene]dichloropalladium(II) (1.08 g, 1.48 mmol) under nitrogen atmosphere. The mixture was stirred at 100° C. for 1 h under nitrogen atmosphere. The reaction mixture was cooled to ambient temperature. Saturated potassium fluoride (20 mL) was added to the reaction mixture. Then the mixture was extracted with ethyl acetate (3×20 mL). The combined organic layers were washed with brine (3×20 mL), dried over sodium sulfate, filtered and concentrated under reduced pressure to afford the title compound as a black solid (3.10 g, crude): ¹H NMR (400 MHz, CDCl₃) δ 8.73 (d, J=4.8 Hz, 1H), 7.33 (d, J=4.8 Hz, 1H), 7.15 (t, J=6.0 Hz, 2H), 7.01 (t, J=6.8 Hz, 1H), 5.23 (d, J=2.4 Hz, 1H), 4.58 (d, J=2.4 Hz, 1H), 3.90 (q, J=7.2 Hz, 2H), 1.30 (t, J=7.2 Hz, 3H).

### Step 2. Preparation of 1-(4-(2,5-difluorophenyl)-3-nitropyridin-2-yl)ethan-1-one

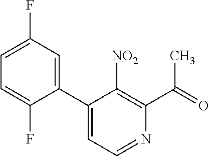 [SMILES: CC(=O)c1nccc(-c2cc(F)ccc2F)c1[N+](=O)[O-]]

To a solution of 4-(2,5-difluorophenyl)-2-(1-ethoxyvinyl)-3-nitropyridine (3.10 g, 10.1 mmol) in tetrahydrofuran (100 mL) was added hydrochloric acid (12 M, 10 mL). The mixture was stirred at 25° C. for 12 h. The pH was adjusted to 7 with saturated sodium bicarbonate. Water (10 mL) was added to the mixture, then the mixture was extracted with ethyl acetate (3×20 mL). The combined organic layers were washed with brine (3×10 mL), dried over sodium sulfate, filtered, and concentrated under reduced pressure. The residue was purified by flash silica gel chromatography, eluting with a gradient of 0-20% of ethyl acetate in petroleum ether, to afford the title compound as a yellow solid (1.10 g, 39% yield): ¹H NMR (400 MHz, CDCl₃) δ 8.83 (d, J=4.8 Hz, 1H), 7.58 (dd, J=4.8, 0.8 Hz, 1H), 7.21-7.14 (m, 2H), 7.02 (tdd, J=7.6, 5.6, 1.6 Hz, 1H), 2.79 (s, 3H).

### Step 3. Preparation of 1-(3-amino-4-(2,5-difluorophenyl)pyridin-2-yl)ethan-1-one

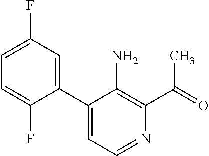 [SMILES: CC(=O)c1nccc(-c2cc(F)ccc2F)c1N]

To a solution of 1-(4-(2,5-difluorophenyl)-3-nitropyridin-2-yl)ethan-1-one (1.10 g, 3.95 mmol) in ethanol (100 mL) and water (10 mL) was added iron powder (5.96 g, 107 mmol) and acetic acid (14.3 g, 237 mmol). The mixture was stirred at 80° C. for 12 h. After cooling to ambient temperature, the resulting mixture was filtered over Celite. The residue was purified by flash silica gel chromatography, eluting with a gradient of 0-25% of ethyl acetate in petroleum ether, to afford the title compound as a yellow solid (0.700 g, 57% yield): ¹H NMR (400 MHz, CDCl₃) δ 8.07 (d, J=4.4 Hz, 1H), 7.24-7.17 (m, 1H), 7.17-7.11 (m, 2H), 7.07 (ddd, J=3.2, 5.2, 8.4 Hz, 1H), 6.32 (s, 2H), 2.76 (s, 3H).

### Step 4. Preparation of 2-(1-(3,3-difluoroazetidin-1-yl)ethyl)-4-(2,5-difluorophenyl)pyridin-3-amine

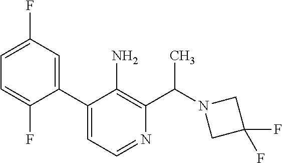 [SMILES: CC(c1nccc(-c2cc(F)ccc2F)c1N)N1CC(F)(F)C1]

To a solution of 1-(3-amino-4-(2,5-difluorophenyl)pyridin-2-yl)ethan-1-one (0.600 g, 2.42 mmol) in 1,2-dichloroethane (10 mL) was added N,N-diisopropylethylamine (0.625 g, 4.83 mmol) and 3,3-difluoroazetidine hydrochloride (0.470 g, 3.63 mmol), and the mixture was stirred at 50° C. for 1 h. Sodium triacetoxyborohydride (2.56 g, 12.1 mmol) was added and the mixture was stirred at 50° C. for 12 h. The reaction mixture was cooled to ambient temperature. Water (10 mL) was added to the mixture, then it was extracted with ethyl acetate (3×20 mL). The combined organic layers were washed with brine (3×10 mL), dried over sodium sulfate, filtered, and concentrated under reduced pressure. The residue was purified by flash silica gel chromatography, eluting with a gradient of 0-40% of ethyl acetate in petroleum ether, to afford the title compound as a yellow solid (0.320 g, 41% yield): ¹H NMR (400 MHz, CDCl₃) δ 7.94 (d, J=4.8 Hz, 1H), 7.23-7.05 (m, 3H), 6.95 (d, J=4.8 Hz, 1H), 5.64-4.83 (m, 2H), 3.90 (quartet, J=6.8 Hz, 1H), 3.74-3.61 (m, 2H), 3.61-3.48 (m, 2H), 1.41 (d, J=6.8 Hz, 3H).

### Step 5. Preparation of N-(2-(1-(3,3-difluoroazetidin-1-yl)ethyl)-4-(2,5-difluorophenyl)pyridin-3-yl)-2-isopropylpyrimidine-5-carboxamide

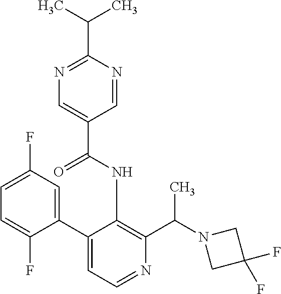 [SMILES: CC(C)c1ncc(C(=O)Nc2c(-c3cc(F)ccc3F)ccnc2C(C)N2CC(F)(F)C2)cn1]

In a similar manner as described in examples disclosed herein, utilizing the appropriately substituted starting materials and intermediates, the title compound was afforded as a colorless solid (0.0500 g, 15% yield): ¹H NMR (400 MHz, DMSO-d₆) δ 10.47 (s, 1H), 8.96 (s, 2H), 8.66 (d, J=4.8 Hz, 1H), 7.44 (d, J=4.8 Hz, 1H), 7.40-7.33 (m, 1H), 7.33-7.24 (m, 2H), 4.11 (quartet, J=6.4 Hz, 1H), 3.71-3.48 (m, 4H), 3.26-3.15 (m, 1H), 1.29 (d, J=7.2 Hz, 6H), 1.25 (d, J=6.4 Hz, 3H).

### Step 6. Preparation of (R) and (S)—N-(2-(1-(3,3-difluoroazetidin-1-yl) ethyl)-4-(2,5-difluorophenyl)pyridin-3-yl)-2-isopropylpyrimidine-5-carboxamide formate salt

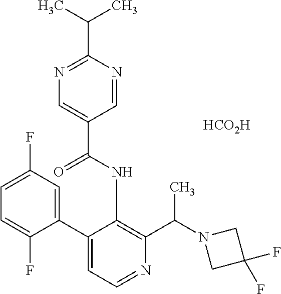 [SMILES: CC(C)c1ncc(C(=O)Nc2c(-c3cc(F)ccc3F)ccnc2C(C)N2CC(F)(F)C2)cn1 ; "HCO2H"]

N-(2-(1-(3,3-difluoroazetidin-1-yl)ethyl)-4-(2,5-difluorophenyl)pyridin-3-yl)-2-isopropylpyrimidine-5-carboxamide (0.0750 g, 0.158 mmol) was purified by chiral SFC (column: DAICEL CHIRALPAK IC (250 mm×30 mm, 5 μm), eluting with 15% of isopropanol containing 0.1% ammonium hydroxide in supercritical carbon dioxide, to afford peak 1 (retention time=0.883 min) as a colorless solid (0.0200 g, 14% yield, 99% ee). The residue was purified by preparative HPLC, eluting with a gradient of 10-40% of acetonitrile in water containing 0.225% formic acid, to afford the title compound as a colorless solid (0.0106 g, 14% yield): ¹H NMR (400 MHz, DMSO-d₆) δ 10.50 (s, 0.5H), 8.96 (s, 2H), 8.65 (d, J=4.8 Hz, 1H), 8.47 (s, 0.42H), 7.43 (d, J=4.4 Hz, 1H), 7.40-7.20 (m, 3H), 4.15-4.07 (m, 1H), 3.69-3.49 (m, 4H), 3.20 (dt, J=13.6, 6.8 Hz, 1H), 1.29 (d, J=6.8 Hz, 6H), 1.24 (d, J=6.4 Hz, 3H); MS (ES+) m/z 474.2 (M+1).

Peak 2 (retention time=0.979) afforded a colorless solid (0.0250 g, 96% ee). This residue was purified by preparative HPLC, eluting with a gradient of 10-40% of acetonitrile in water containing 0.225% formic acid, to afford the title compound as a colorless solid (0.0115 g, 15% yield): ¹H NMR (400 MHz, DMSO-d₆) δ 10.48 (s, 0.5H), 8.96 (s, 2H), 8.65 (d, J=4.8 Hz, 1H), 8.36 (s, 0.1H), 7.43 (d, J=4.8 Hz, 1H), 7.40-7.23 (m, 3H), 4.10 (q, J=6.4 Hz, 1H), 3.68-3.49 (m, 4H), 3.20 (dt, J=13.8, 8.0 Hz, 1H), 1.28 (d, J=6.8 Hz, 6H), 1.24 (d, J=6.4 Hz, 3H); MS (ES+) m/z 474.3 (M+1).

In a similar manner as described in examples disclosed herein, utilizing the appropriately substituted starting materials, intermediates, and chiral separation conditions, the following compounds were prepared:

| | | Amount | | |
|---|---|---|---|---|
| Example | Structure | Yield, | | ¹H NMR, MS (ES+) |
| No. | Name | % ee | Condition | Rt |
| 214 | 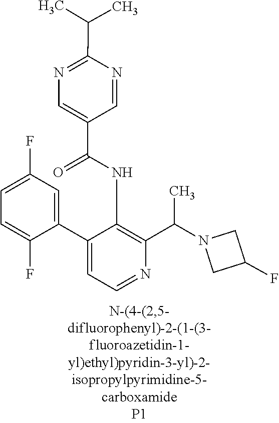 [SMILES: CC(C)c1ncc(C(=O)Nc2c(-c3cc(F)ccc3F)ccnc2C(C)N2CC(F)C2)cn1] | 0.013 g 18%, 100% ee | column: DAICEL CHIRALPAK 1C (250 mm × 30 mm, 10 μm) eluting with 15% of isopropanol containing 0.1% ammonium hydroxide in supercritical carbon dioxide | ¹H NMR (400 MHz, CDCl₃) δ 11.53 (s, 1H), 9.03 (s, 2H), 8.46 (d, J = 5.2 Hz, 1H), 7.26 (s, 1H), 7.12 (s, 1H), 7.08- 6.95 (m, 2H), 5.39- 5.18 (m, 1H), 3.96 (q, J = 6.8 Hz, 1H), 3.79-3.56 (m, 2H), 3.52-3.37 (m, 2H), 3.30 (td, J = 6.8, 13.6 Hz, 1H), 1.39 (d, J = 6.8 Hz, 6H), 1.31 (d, J = 6.8 Hz, 3H); MS (ES+) m/z 456.2 (M + 1); Rₜ = 0.971 min |
| 215 | 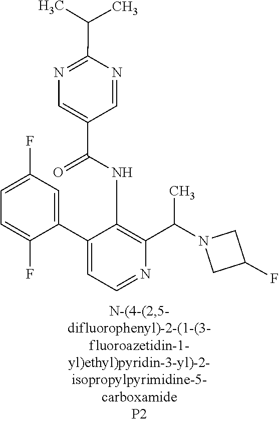 [SMILES: CC(C)c1ncc(C(=O)Nc2c(-c3cc(F)ccc3F)ccnc2C(C)N2CC(F)C2)cn1] | 0.0178 g 43%, 97.9% ee | | ¹H NMR (400 MHz, CDCl₃) δ 11.52 (s, 1H), 9.03 (s, 2H), 8.46 (d, J = 4.8 Hz, 1H), 7.26 (s, 1H), 7.12 (s, 1H), 7.07- 6.96 (m, 2H), 5.40- 5.17 (m, 1H), 3.97 (q, J = 6.8 Hz, 1H), 3.82-3.55 (m, 2H), 3.52-3.37 (m, 2H), 3.30 (dt, J = 14.0, 6.8 Hz, 1H), 1.39 (d, J = 6.8 Hz, 6H), 1.31 (d, J = 6.8 Hz, 3H); MS (ES+) m/z 456.2 (M + 1), Rₜ = 1.164 min |
| 216 | 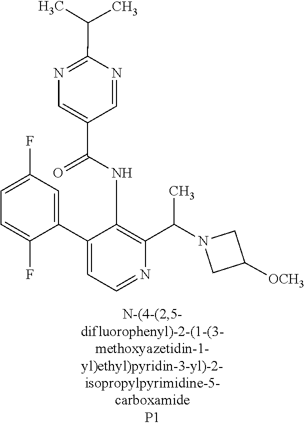 [SMILES: COC1CN(C(C)c2nccc(-c3cc(F)ccc3F)c2NC(=O)c2cnc(C(C)C)nc2)C1] | 0.010 g 14%, 98% ee | column: DAICEL CHIRALPAK IC (250 mm × 30 mm, 10 μm) eluting with 15% of isopropanol containing 0.1% ammonium hydroxide in supercritical carbon dioxide | ¹H NMR (400 MHz, CDCl₃) δ 12.25- 11.78 (m, 1H), 9.06 (s, 2H), 8.44 (d, J = 5.2 Hz, 1H), 7.25 (d, J = 4.8 Hz, 1H), 7.15-7.08 (m, 1H), 7.08-7.01 (m, 1H), 7.01-6.94 (m, 1H), 4.21-4.16 (m, 1H), 3.95-3.90 (m, 1H), 3.70-3.67 (m, 1H), 3.56-3.53 (m, 1H), 3.35 (s, 3H), 3.33- 3.25 (m, 1H), 3.23- 3.11 (m, 2H), 1.39 (d, J = 6.8 Hz, 6H), 1.28 (d, J = 6.8 Hz, 3H); MS (ES+) m/z 468.0 (M + 1); Rₜ = 1.334 min |
| 217 | 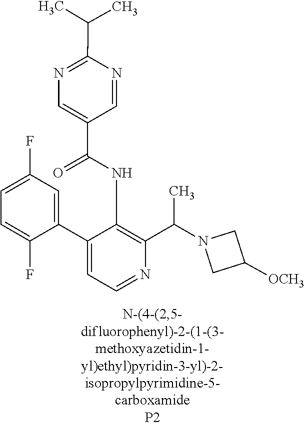 [SMILES: COC1CN(C(C)c2nccc(-c3cc(F)ccc3F)c2NC(=O)c2cnc(C(C)C)nc2)C1] | 0.030 g 42%, 93% ee | | ¹H NMR (400 MHz, CDCl₃) δ 12.12- 12.00 (m, 1H), 9.07 (s, 2H), 8.44 (d, J = 4.8 Hz, 1H), 7.25 (d, J = 5.2 Hz, 1H), 7.15-7.06 (m, 1H), 7.08-7.02 (m, 1H), 7.01-6.95 (m, 1H), 4.21-4.16 (m, 1H), 3.95-3.89 (m, 1H), 3.73-3.64 (m, 1H), 3.58-3.50 (m, 1H), 3.35 (s, 3H), 3.33- 3.25 (m, 1H), 3.24- 3.13 (m, 2H), 1.39 (d, J = 6.8 Hz, 6H), 1.28 (d, J = 6.8 Hz, 3H); MS (ES+) m/z 468.0 (M + 1); Rₜ = 1.480 min |
| 218 | 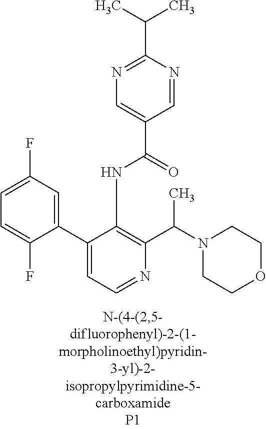 [SMILES: CC(C)c1ncc(C(=O)Nc2c(-c3cc(F)ccc3F)ccnc2C(C)N2CCOCC2)cn1] | 0.013 g 34%, 99% ee | column: DAICEL CHIRALPAK AD (250 mm × 30 mm, 10 μm) eluting with 25% of isopropanol containing 0.1% ammonium hydroxide in supercritical carbon dioxide | ¹H NMR (400 MHz, CDCl₃) δ 11.56- 11.32 (m, 1H), 9.04 (s, 2H), 8.50 (d, J = 5.2 Hz, 1H), 7.26- 7.24 (m, 1H), 7.18- 7.11 (m, 1H), 7.06- 6.96 (m, 2H), 3.96- 3.89 (m, 1H), 3.87- 3.74 (m, 4H), 3.33- 3.26 (m, 1H) 2.69- 2.60 (m, 2H), 257- 2.48 (m, 2H), 1.45 (d, J = 6.8 Hz, 3H), 1.38 (d, J = 6.8 Hz, 6H); MS (ES+) m/z 468.0 (M + 1); Rₜ = 1.192 min |
| 219 | 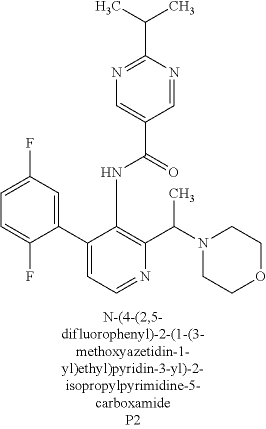 [SMILES: CC(C)c1ncc(C(=O)Nc2c(-c3cc(F)ccc3F)ccnc2C(C)N2CCOCC2)cn1] | 0.030 g 33%, 99% ee | | ¹H NMR (400 MHz, CDCl₃) δ 11.57- 11.18 (m, 1H), 9.05 (s, 2H), 8.51 (d, J = 4.8 Hz, 1H), 7.30- 7.27 (m, 1H), 7.21- 7.08 (m, 1H), 7.07- 6.93 (m, 2H), 4.08- 3.94 (m, 1H), 3.92- 3.71 (m, 4H), 3.32- 3.25 (m, 1H), 2.88- 2.62 (m, 2H), 2.62- 2.39 (m, 2H), 1.46 (d, J = 6.0 Hz, 3H), 1.37 (d, J = 7.2 Hz, 6H); MS (ES+) m/z 468.0 (M + 1); Rₜ = 1.324 min |

### Example 220

### Synthesis of N-(4-(2,5-difluorophenyl)-2-(4-fluorophenyl) pyridin-3-yl)-2-isopropylpyrimidine-5-carboxamide

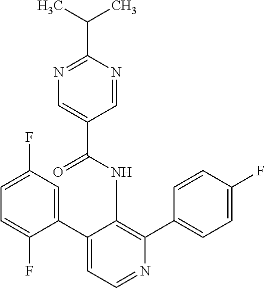 [SMILES: CC(C)c1ncc(C(=O)Nc2c(-c3cc(F)ccc3F)ccnc2-c2ccc(F)cc2)cn1]

### Step 1. Preparation of 2-chloro-4-(2,5-difluorophenyl)-3-nitropyridine & 4-chloro-2-(2,5-difluorophenyl)-3-nitropyridine

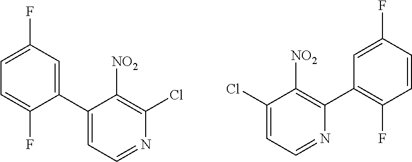 [SMILES: O=[N+]([O-])c1c(-c2cc(F)ccc2F)ccnc1Cl ; O=[N+]([O-])c1c(Cl)ccnc1-c1cc(F)ccc1F]

To a solution of 2,4-dichloro-3-nitropyridine (40.0 g, 207 mmol), (2,5-difluorophenyl)boronic acid (32.7 g, 207 mmol) and potassium carbonate (85.9 g, 622 mmol) in dioxane (600 mL) and water (200 mL) was added [1,1′-bis(diphenylphosphino)ferrocene]dichloropalladium(II) (7.58 g, 10.4 mmol) at 25° C. under a nitrogen atmosphere. The mixture was stirred at 60° C. for 45 min. After being cooling to ambient temperature, the mixture was diluted with ethyl acetate (200 mL). The mixture was filtered, and the filtrate was washed with brine (3×200 mL), dried over sodium sulfate, filtered, and concentrated under reduced pressure. The residue was purified by reverse phase column chromatography, eluting with 0.1% formic acid, and then purified by special normal-phase column chromatography, eluting with a gradient of 1-15% of ethanol containing 0.1% formic acid in hexanes to afford 2-chloro-4-(2,5-difluorophenyl)-3-nitropyridine as a yellow solid (column: Welch Ultimate XB-CN 250 mm×50 mm×10 μm) (21.9 g, 19% yield): ¹H NMR (400 MHz, MeOD) δ 8.78 (dd, J=5.2, 2.0 Hz, 1H), 7.88-7.79 (m, 1H), 7.39-7.19 (m, 3H). Minor byproduct 4-chloro-2-(2,5-difluorophenyl)-3-nitropyridine was afforded as a yellow solid (5.30 g, 5% yield): ¹H NMR (400 MHz, MeOD) δ8.66 (d, J=5.2 Hz, 1H), 7.62 (dd, J=5.2, 0.8 Hz, 1H), 7.38-7.26 (m, 2H), 7.25-7.17 (m, 1H).

### Step 2. Preparation of 4-(2,5-difluorophenyl)-2-(4-fluorophenyl)-3-nitropyridine

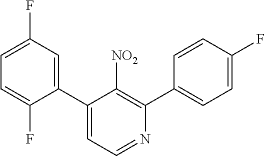 [SMILES: O=[N+]([O-])c1c(-c2cc(F)ccc2F)ccnc1-c1ccc(F)cc1]

To a solution of 2-chloro-4-(2,5-difluorophenyl)-3-nitropyridine (0.217 g, 0.802 mmol), (4-fluorophenyl)boronic acid (0.169 g, 1.21 mmol) and potassium carbonate (0.334 g, 2.42 mmol) in dioxane (3 mL) and water (1 mL) was added [1,1′-bis(diphenylphosphino)ferrocene]dichloropalladium(II) (0.0598 g, 0.0817 mmol) at 25° C. under a nitrogen atmosphere. The mixture was stirred at 60° C. for 1 h. After being cooling to ambient temperature, the mixture was concentrated under reduced pressure. The residue was purified by flash silica gel chromatography, eluting with a gradient of 0-22% of ethyl acetate in petroleum ether, to afford the title compound as a colorless solid (0.260 g, 80% yield): ¹H NMR (400 MHz, DMSO-d₆) δ 9.00 (d, J=5.2 Hz, 1H), 7.78 (d, J=5.2 Hz, 1H), 7.62 (dd, J=8.8, 5.2 Hz, 2H), 7.52-7.43 (m, 3H), 7.42-7.33 (m, 2H).

### Step 3. Preparation of 4-(2,5-difluorophenyl)-2-(4-fluorophenyl) pyridin-3-amine

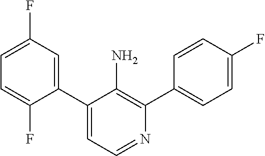 [SMILES: Nc1c(-c2cc(F)ccc2F)ccnc1-c1ccc(F)cc1]

To a solution of 4-(2,5-difluorophenyl)-2-(4-fluorophenyl)-3-nitropyridine (0.100 g, 0.303 mmol) in methanol (10 mL) was added palladium on carbon (20 mg, 10 wt %) at 25° C. under nitrogen atmosphere. The suspension was degassed and purged with hydrogen three times. The mixture was stirred at 25° C. for 12 h under hydrogen (15 psi, balloon). After completion of the reaction, the resulting mixture was filtered over Celite. The filter cake was washed with methanol (20 mL). The filtrate was concentrated under reduced pressure to afford the title compound as a colorless solid (0.0900 g, 94% yield): ¹H NMR (400 MHz, DMSO-d₆) δ 7.98 (d, J=4.8 Hz, 1H), 7.70 (dd, J=8.8, 5.6 Hz, 2H), 7.46-7.38 (m, 1H), 7.37-7.27 (m, 4H), 7.03 (d, J=4.4 Hz, 1H), 4.69-4.61 (m, 2H).

### Step 4. Preparation of N-(4-(2,5-difluorophenyl)-2-(4-fluorophenyl) pyridin-3-yl)-2-isopropylpyrimidine-5-carboxamide

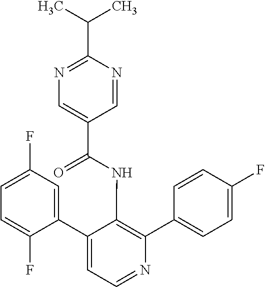 [SMILES: CC(C)c1ncc(C(=O)Nc2c(-c3cc(F)ccc3F)ccnc2-c2ccc(F)cc2)cn1]

In a similar manner as described in examples disclosed herein, utilizing the appropriately substituted starting materials and intermediates, the title compound was afforded as a colorless solid (0.0400 g, 23% yield): ¹H NMR (400 MHz, DMSO-d₆) δ 10.50 (s, 1H), 8.81 (s, 2H), 8.76 (d, J=4.8 Hz, 1H), 7.75-7.67 (m, 2H), 7.55 (d, J=4.8 Hz, 1H), 7.38 (td, J=9.6, 4.4 Hz, 1H), 7.34-7.23 (m, 4H), 3.16 (dt, J=13.6, 6.8 Hz, 1H), 1.25 (d, J=6.8 Hz, 6H); MS (ES+) m/z 449.1 (M+1).

### Example 221

### Synthesis of N-(2,4-bis(4,4-difluorocyclohexyl)pyridin-3-yl)-2-isopropylpyrimidine-5-carboxamide

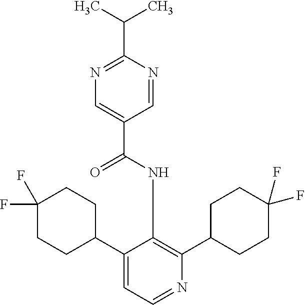 [SMILES: CC(C)c1ncc(C(=O)Nc2c(C3CCC(F)(F)CC3)ccnc2C2CCC(F)(F)CC2)cn1]

### Step 1. Preparation of 2,4-bis(4,4-difluorocyclohex-1-en-1-yl)-3-nitropyridine

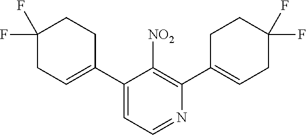 [SMILES: O=[N+]([O-])c1c(C2=CCC(F)(F)CC2)ccnc1C1=CCC(F)(F)CC1]

To a solution of 2,4-dichloro-3-nitropyridine (0.100 g, 0.518 mmol), 2-(4,4-difluorocyclohex-1-en-1-yl)-4,4,5,5-tetramethyl-1,3,2-dioxaborolane (0.253 g, 1.04 mmol) and potassium carbonate (0.179 g, 1.30 mmol,) in dioxane (10 mL) and water (1 mL) was added [1,1′-bis(diphenylphosphino)ferrocene]dichloropalladium(II) (0.0379 g, 0.0518 mmol) under a nitrogen atmosphere. The mixture was stirred at 80° C. for 12 h under a nitrogen atmosphere. The reaction mixture was cooled to ambient temperature and filtered over Celite. The filtrate was concentrated under reduced pressure. The residue was purified by flash silica gel chromatography, eluting with a gradient of 9-33% of ethyl acetate in petroleum ether, to afford the title compound as a yellow oil (0.0700 g, 35% yield): ¹H NMR (400 MHz, CDCl₃) δ 8.62 (d, J=5.2 Hz, 1H), 7.15 (d, J=5.2 Hz, 1H), 5.85-5.78 (m, 1H), 5.66 (s, 1H), 2.81-2.74 (m, 3H), 2.68 (t, J=14.0 Hz, 6H), 2.61-2.52 (m, 3H), 2.26-2.10 (m, 6H).

### Step 2. Preparation of 2,4-bis(4,4-difluorocyclohexyl)pyridin-3-amine

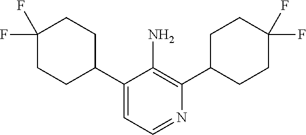 [SMILES: Nc1c(C2CCC(F)(F)CC2)ccnc1C1CCC(F)(F)CC1]

To a solution of 2,4-bis(4,4-difluorocyclohex-1-en-1-yl)-3-nitropyridine (0.0700 g, 0.196 mmol) in methanol (5 mL) was added palladium on carbon (0.0350 g, 10 wt %) under nitrogen atmosphere. The suspension was degassed and purged with hydrogen for 3 times. The mixture was stirred under hydrogen (15 psi, balloon) at 25° C. for 12 h. The reaction mixture was filtered through a pad of Celite and the filtrate was concentrated under reduced pressure to afford the title compound as a yellow oil (0.0600 g, crude): ¹H NMR (400 MHz, MeOD) δ 7.85 (d, J=6.0 Hz, 1H), 7.55 (d, J=5.6 Hz, 1H), 3.40-3.41 (m, 1H), 3.09-3.03 (t, J=12.0 Hz, 1H), 2.32-2.09 (m, 6H), 2.06-1.95 (m, 8H), 1.83-1.68 (m, 2H).

### Step 3. Preparation of N-(2,4-bis(4,4-difluorocyclohexyl)pyridin-3-yl)-2-isopropylpyrimidine-5-carboxamide

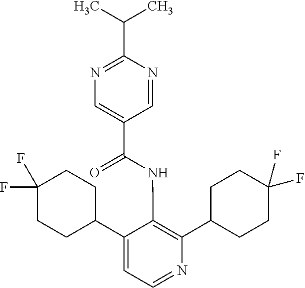 [SMILES: CC(C)c1ncc(C(=O)Nc2c(C3CCC(F)(F)CC3)ccnc2C2CCC(F)(F)CC2)cn1]

In a similar manner as described in other examples disclosed herein (e.g., Example 186, step 1), utilizing the appropriately substituted starting materials and intermediates, the title compound was afforded as a colorless solid (0.00950 g, 16% yield): ¹H NMR (400 MHz, DMSO-d₆) δ 10.3 (d, J=3.2 Hz, 1H), 9.26 (s, 2H), 8.46 (d, J=4.0 Hz, 1H), 7.30 (dd, J=4.8, 2.8 Hz, 1H), 3.26 (dt, J=13.6, 6.8 Hz, 1H), 3.14-3.01 (m, 1H), 2.98-2.85 (m, 1H), 2.12-1.96 (m, 5H), 1.95-1.70 (m, 9H), 1.69-1.55 (m, 2H), 1.34 (d, J=6.8 Hz, 6H); MS (ES+) m/z 479.3 (M+1).

### Example 222

In a similar manner as described in other examples disclosed herein (e.g., Example 221), utilizing the appropriately substituted starting materials and intermediates, the following compounds were prepared:

| Example | Structure | | MS (ES+) | |
|---|---|---|---|---|
| No. | Name | Yield | m/z | ¹H-NMR |
| 222 | 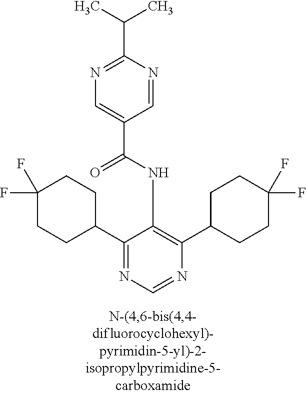 [SMILES: CC(C)c1ncc(C(=O)Nc2c(C3CCC(F)(F)CC3)ncnc2C2CCC(F)(F)CC2)cn1] | 48% | 480.3 (M + 1) | ¹H NMR (400 MHz, DMSO-d₆) δ 10.46 (s, 1H), 9.28 (s, 2H), 9.05 (s, 1H), 3.30-3.23 (m, 1H), 3.19-3.06 (m, 2H), 2.11-1.75 (m, 16H), 1.35 (d, J = 6.8 Hz, 6H) |

### Example 223

### Synthesis of N-(2-(sec-butyl)-4-(2,5-difluorophenyl) pyridin-3-yl)-2-isopropylpyrimidine-5-carboxamide

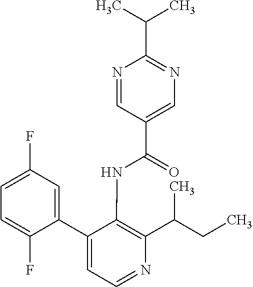 [SMILES: CCC(C)c1nccc(-c2cc(F)ccc2F)c1NC(=O)c1cnc(C(C)C)nc1]

### Step 1. Preparation of (E)-2-(but-2-en-2-yl)-4-(2,5-difluorophenyl) pyridin-3-amine

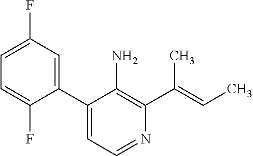 [SMILES: C/C=C(\C)c1nccc(-c2cc(F)ccc2F)c1N]

To a solution of 2-chloro-4-(2,5-difluorophenyl)pyridin-3-amine (0.120 g, 0.499 mmol) in dioxane (2 mL) and water (0.4 mL) was added (Z)-2-(but-2-en-2-yl)-4,4,5,5-tetramethyl-1,3,2-dioxaborolane (0.0908 g, 0.499 mmol), [1,1′-bis(diphenylphosphino)ferrocene]dichloropalladium(II) (0.0365 g, 0.0499 mmol) and potassium carbonate (0.207 g, 1.50 mmol). The mixture was stirred at 80° C. for 12 h under a nitrogen atmosphere. The reaction mixture was cooled to ambient temperature and concentrated under reduced pressure. The residue was purified by flash silica gel chromatography, eluting with a gradient of 0-24% of ethyl acetate in petroleum ether, to afford the title compound as a yellow oil (0.0900 g, 69% yield): ¹H NMR (400 MHz, CDCl₃) δ 8.06 (d, J=4.8 Hz, 1H), 7.21-7.07 (m, 3H), 6.90 (d, J=4.8 Hz, 1H), 5.86 (dq, J=1.2, 6.8 Hz, 1H), 3.87 (s, 2H), 2.08 (s, 3H), 1.86 (d, J=6.8 Hz, 1H).

### Step 2. Preparation of 2-(sec-butyl)-4-(2,5-difluorophenyl) pyridin-3-amine

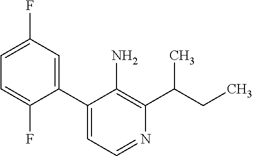 [SMILES: CCC(C)c1nccc(-c2cc(F)ccc2F)c1N]

To a solution of (E)-2-(but-2-en-2-yl)-4-(2,5-difluorophenyl)pyridin-3-amine (0.0900 g, 0.346 mmol) in methanol (5 mL) was added palladium on carbon (0.0700 g, 10 wt %) under a nitrogen atmosphere. The suspension was degassed under vacuum and purged with hydrogen several times. The mixture was stirred under hydrogen (15 psi, balloon) at 25° C. for 12 h. The reaction mixture was filtered over Celite and the filtrate was concentrated under reduced pressure to afford the title compound as a colorless oil (0.0700 g, 77% yield): ¹H NMR (400 MHz, CDCl₃) δ 8.11 (d, J=4.8 Hz, 1H), 7.22-7.15 (m, 1H), 7.14-7.07 (m, 2H), 6.88 (d, J=4.8 Hz, 1H), 3.68 (s, 2H), 2.84 (sextet, J=6.8 Hz, 1H), 1.99-1.85 (m, 1H), 1.68 (dquintet, J=14.0, 7.2 Hz, 1H), 1.33 (d, J=6.8 Hz, 3H), 0.94 (t, J=7.2 Hz, 3H).

### Step 3. Preparation of N-(2-(sec-butyl)-4-(2,5-difluorophenyl) pyridin-3-yl)-2-isopropylpyrimidine-5-carboxamide

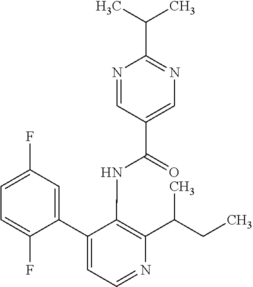 [SMILES: CCC(C)c1nccc(-c2cc(F)ccc2F)c1NC(=O)c1cnc(C(C)C)nc1]

In a similar manner as described in other examples disclosed herein (e.g., Example 186, step 1), utilizing the appropriately substituted starting materials and intermediates, the title compound was afforded as a yellow solid (0.0402 g, 36% yield): ¹H NMR (400 MHz, DMSO-d₆) δ 10.35 (s, 1H), 8.95 (s, 2H), 8.63 (d, J=4.8 Hz, 1H), 7.39-7.31 (m, 2H), 7.30-7.21 (m, 2H), 3.26-3.07 (m, 2H), 1.84-1.67 (m, 1H), 1.62-1.44 (m, 1H), 1.27 (d, J=6.8 Hz, 6H), 1.18 (d, J=5.6 Hz, 3H), 0.75 (t, J=6.8 Hz, 3H); MS (ES+) m/z 411.2 (M+1).

### Example 224

### Synthesis of N-(2-(5,5-difluoro-1,3-dioxan-2-yl)-4-(2,5-difluorophenyl)pyridin-3-yl)-2-isopropylpyrimidine-5-carboxamide

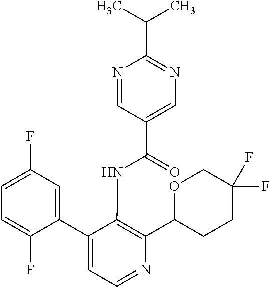 [SMILES: CC(C)c1ncc(C(=O)Nc2c(-c3cc(F)ccc3F)ccnc2C2CCC(F)(F)CO2)cn1]

### Step 1. Preparation of 4-(2,5-difluorophenyl)-3-nitro-2-vinylpyridine

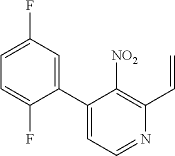 [SMILES: C=Cc1nccc(-c2cc(F)ccc2F)c1[N+](=O)[O-]]

To a solution of 2-chloro-4-(2,5-difluorophenyl)-3-nitropyridine (3.00 g, 11.1 mmol), [1,1′-bis(diphenylphosphino)ferrocene] dichloropalladium(II) (0.81 g, 1.10 mmol) and potassium carbonate (4.60 g, 33.3 mmol) in dioxane (40 mL) and water (10 mL) under a nitrogen atmosphere was added 4,4,5,5-tetramethyl-2-vinyl-1,3,2-dioxaborolane (8.54 g, 55.4 mmol). The mixture was stirred at 90° C. for 3 h. The mixture was cooled to 25° C. and concentrated under reduced pressure. The residue was purified by flash silica gel chromatography, eluting with a gradient of 35-50% of ethyl acetate in petroleum ether, to afford the title compound as a yellow oil (1.30 g, 42% yield): ¹H NMR (400 MHz, DMSO-d₆) δ 8.92 (d, J=4.8 Hz, 1H), 7.68 (d, J=4.8 Hz, 1H), 7.52-7.40 (m, 3H), 6.83 (dd, J=16.6, 10.4 Hz, 1H), 6.61 (dd, J=16.4, 2.0 Hz, 1H), 5.81 (dd, J=10.4, 2.0 Hz, 1H); MS (ES+) m/z 263.1 (M+1).

### Step 2. Preparation of 4-(2,5-difluorophenyl)-3-nitropicolinaldehyde

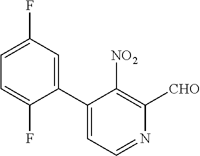 [SMILES: O=Cc1nccc(-c2cc(F)ccc2F)c1[N+](=O)[O-]]

A solution of 4-(2,5-difluorophenyl)-3-nitro-2-vinylpyridine (1.30 g, 4.96 mmol) in dichloromethane (45 mL) at −78° C. was treated with ozone for 15 min. Excess ozone was removed by blowing nitrogen. Triphenylphosphine (3.90 g, 14.8 mmol) was added at −78° C. The mixture was warmed to 25° C. and stirred at 25° C. for 1 h. The residue was purified by flash silica gel chromatography, eluting with a gradient of 60-75% of ethyl acetate in petroleum ether, to afford the title compound as a yellow solid (0.550 g, 38% yield): ¹H NMR (400 MHz, DMSO-d₆) δ 10.03 (s, 1H), 9.15 (d, J=5.2 Hz, 1H), 8.09 (d, J=4.8 Hz, 1H), 7.53-7.47 (m, 2H), 7.45-7.38 (m, 1H).

### Step 3. Preparation of 2-(5,5-difluoro-1,3-dioxan-2-yl)-4-(2,5-difluorophenyl)-3-nitropyridine

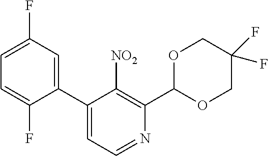 [SMILES: O=[N+]([O-])c1c(-c2cc(F)ccc2F)ccnc1C1OCC(F)(F)CO1]

A solution of 4-(2,5-difluorophenyl)-3-nitropicolinaldehyde (0.0500 g, 0.189 mmol), 2,2-difluoropropane-1,3-diol (0.0220 g, 0.196 mmol) and p-toluene sulfonic acid monohydrate (0.00400 g, 0.0189 mmol) in toluene (1 mL) was stirred at 110° C. for 12 h. The mixture was concentrated under reduced pressure to afford the title compound as a yellow oil (0.0600 g, crude): ¹H NMR (400 MHz, DMSO-d₆) δ 7.48-7.45 (m, 3H), 7.12-7.10 (m, 2H), 6.23-6.20 (m, 1H), 4.31-4.25 (m, 2H), 3.63-3.56 (m, 2H); MS (ES+) m/z 359.1 (M+1).

### Step 4. Preparation of 2-(5,5-difluoro-1,3-dioxan-2-yl)-4-(2,5-difluorophenyl)pyridin-3-amine

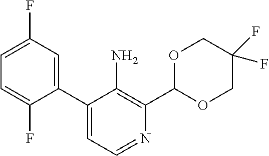 [SMILES: Nc1c(-c2cc(F)ccc2F)ccnc1C1OCC(F)(F)CO1]

To a solution of 2-(5,5-difluoro-1,3-dioxan-2-yl)-4-(2,5-difluorophenyl)-3-nitropyridine (0.0600 g, 0.167 mmol) in methanol (2 mL) was added palladium on carbon (0.0600 g, 10 wt %) at 25° C. under nitrogen atmosphere. The suspension was degassed and purged with hydrogen three times. The mixture was stirred at 25° C. for 12 h under hydrogen (15 psi, balloon). The resulting mixture was filtered over Celite. The filter cake was washed with methanol (20 mL). The filtrate was concentrated under reduced pressure to afford the title compound as a yellow oil (0.0500 g, 81% yield): ¹H NMR (400 MHz, DMSO-d₆) δ 7.48-7.42 (m, 3H), 7.12-7.10 (m, 2H), 5.99 (s, 1H), 4.38-4.36 (m, 2H), 3.63-3.56 (m, 2H); MS (ES+) m/z 329.1 (M+1).

### Step 5. Preparation of N-(2-(5,5-difluoro-1,3-dioxan-2-yl)-4-(2,5-difluorophenyl)pyridin-3-yl)-2-isopropylpyrimidine-5-carboxamide

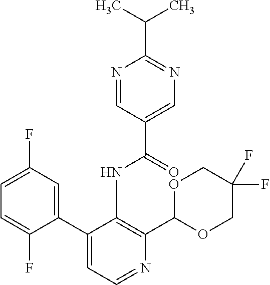 [SMILES: CC(C)c1ncc(C(=O)Nc2c(-c3cc(F)ccc3F)ccnc2C2OCC(F)(F)CO2)cn1]

In a similar manner as described in other examples disclosed herein (e.g., Example 186, step 1), utilizing the appropriately substituted starting materials and intermediates, the title compound was afforded as a yellow solid (0.00750 g, 9% yield): ¹H NMR (400 MHz, DMSO-d₆) δ 10.27 (s, 1H), 8.98 (s, 2H), 8.68 (d, J=4.8 Hz, 1H), 7.61 (d, J=4.4 Hz, 1H), 7.39-7.33 (m, 1H), 7.33-7.24 (m, 2H), 6.01 (s, 1H), 4.26 (s, 2H), 4.24-4.13 (m, 2H), 3.23-3.15 (m, 1H), 1.28 (d, J=6.8 Hz, 6H); MS (ES+) m/z 477.3 (M+1).

### Example 225

### Synthesis of N-(2-(4,4-difluorocyclohexyl)-4-(2,5-difluorophenyl)pyridin-3-yl)-5-isopropoxypyrazine-2-carboxamide

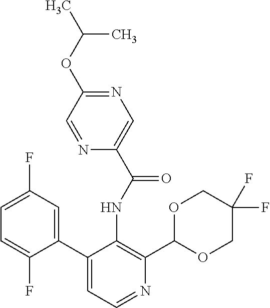 [SMILES: CC(C)Oc1cnc(C(=O)Nc2c(-c3cc(F)ccc3F)ccnc2C2OCC(F)(F)CO2)cn1]

### Step 1. Preparation of 5-chloro-N-(2-(4,4-difluorocyclohexyl)-4-(2,5-difluorophenyl)pyridin-3-yl)pyrazine-2-carboxamide

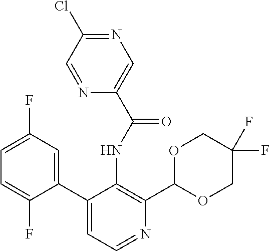 [SMILES: O=C(Nc1c(-c2cc(F)ccc2F)ccnc1C1OCC(F)(F)CO1)c1cnc(Cl)cn1]

In a similar manner as described in other examples disclosed herein (e.g., Example 186, step 1), utilizing the appropriately substituted starting materials and intermediates, the title compound was afforded as a colorless solid (0.0900 g, 47% yield): ¹H NMR (400 MHz, DMSO-d₆) δ 10.61 (s, 1H), 8.93 (dd, J=5.6, 1.2 Hz, 2H), 8.61 (d, J=4.8 Hz, 1H), 7.36 (d, J=4.8 Hz, 1H), 7.32-7.25 (m, 2H), 7.23 (dd, J=7.6, 4.0 Hz, 1H), 3.19-3.01 (m, 1H), 2.08-2.05 (m, 2H) 1.92-1.76 (m, 6H); MS (ES+) m/z 465.2, 467.2 (M+1).

### Step 2. Preparation of N-(2-(4,4-difluorocyclohexyl)-4-(2,5-difluorophenyl)pyridin-3-yl)-5-isopropoxypyrazine-2-carboxamide

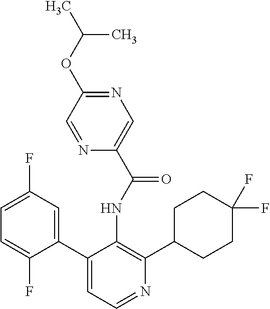 [SMILES: CC(C)Oc1cnc(C(=O)Nc2c(-c3cc(F)ccc3F)ccnc2C2CCC(F)(F)CC2)cn1]

A solution of 5-chloro-N-(2-(4,4-difluorocyclohexyl)-4-(2,5-difluorophenyl)pyridin-3-yl)pyrazine-2-carboxamide (0.0200 g, 0.0430 mmol) and cesium carbonate (0.0420 g, 0.129 mmol) in isopropanol (0.785 g, 0.0130 mmol) was stirred at 80° C. for 12 h. The mixture was cooled to 25° C. and concentrated under reduced pressure. The residue was purified by preparative HPLC, eluting with a gradient of 56-86% of acetonitrile in water containing 0.225% formic acid, to afford the title compound as a colorless solid (0.00730 g, 34% yield): ¹H NMR (400 MHz, DMSO-d₆) δ 10.28 (s, 1H), 8.66 (d, J=1.2 Hz, 1H), 8.59 (d, J=5.2 Hz, 1H), 8.29 (d, J=1.2 Hz, 1H), 7.34 (d, J=4.8 Hz, 1H), 7.29 (td, J=9.2, 4.4 Hz, 2H), 7.24-7.16 (m, 1H), 5.34-5.28 (m, 1H), 3.15-3.06 (m, 1H), 2.08-2.07 (m, 2H), 1.92-1.73 (m, 6H), 1.35 (d, J=6.0 Hz, 6H); MS (ES+) m/z 489.3 (M+1).

### Example 226 and Example 227

### Synthesis of trans-N-(2-(4,4-difluorocyclohexyl)-4-(2,5-difluorophenyl)pyridin-3-yl)-5-methoxy-5-methyltetrahydro-2H-pyran-2-carboxamide P1 and P2/

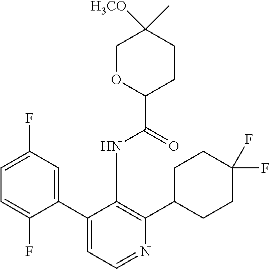 [SMILES: COC1(C)CCC(C(=O)Nc2c(-c3cc(F)ccc3F)ccnc2C2CCC(F)(F)CC2)OC1]

### Step 1. Preparation of trans-6-((benzyloxy)methyl)-3-methyltetrahydro-2H-pyran-3-ol

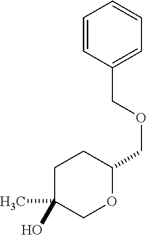

To a mixture of 6-((benzyloxy)methyl)dihydro-2H-pyran-3(4H)-one (3.00 g, 13.6 mmol) in tetrahydrofuran (25 mL) was added methylmagnesium bromide (3 M in tetrahydrofuran, 6.8 mL) at −20° C. under a nitrogen atmosphere. The mixture was stirred at 25° C. for 2 h. The residue was poured into saturated ammonium chloride (20 mL) and extracted with ethyl acetate (3×15 mL). The combined organic layers were dried over anhydrous sodium sulfate, filtered and the filtrate was concentrated in vacuo. The residue was purified by flash silica gel chromatography, eluting with 50% of ethyl acetate in petroleum ether, to afford the title compound as a colorless oil (1.15 g, 36% yield): ¹H NMR (400 MHz, CDCl₃) δ 7.34 (s, 5H), 4.62-4.54 (m, 2H), 3.55-3.38 (m, 4H), 2.11 (s, 3H), 1.74-1.39 (m, 5H).

### Step 2. Preparation of trans-2-((benzyloxy)methyl)-5-methoxy-5-methyltetrahydro-2H-pyran

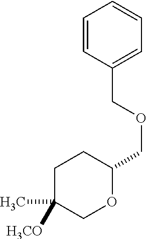

To a mixture of trans-6-((benzyloxy)methyl)-3-methyltetrahydro-2H-pyran-3-ol (1.00 g, 4.23 mmol) in tetrahydrofuran (12 mL) was added sodium hydride (0.254 g, 6.35 mmol, 60% purity) at 0° C. under a nitrogen atmosphere. The mixture was stirred at 25° C. for 0.5 h and then methyl iodide (1.20 g, 8.46 mmol) was added at 0° C. The mixture was stirred at 25° C. for 12 h. The reaction mixture was diluted with water (10 mL) and extracted with ethyl acetate (2×20 mL). The combined organic layers were washed with brine (2×20 mL), dried over sodium sulfate, filtered, and concentrated under reduced pressure. The residue was purified by flash silica gel chromatography, eluting with 50% of ethyl acetate in petroleum ether, to afford the title compound as a colorless oil (0.520 g, 49% yield): ¹H NMR (400 MHz, CDCl₃) δ 7.39-7.32 (m, 4H), 7.32-7.28 (m, 1H), 4.65-4.50 (m, 2H), 3.70 (dd, J=10.8, 2.4 Hz, 1H), 3.58-3.48 (m, 2H), 3.47-3.41 (m, 1H), 3.30 (d, J=10.8 Hz, 1H), 3.26 (s, 3H), 1.97-1.89 (m, 1H), 1.68-1.61 (m, 1H), 1.58-1.54 (m, 1H), 1.49-1.38 (m, 1H), 1.30 (s, 3H).

### Step 3. Preparation of (trans-5-methoxy-5-methyltetrahydro-2H-pyran-2-yl)methanol

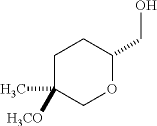

To a mixture of trans-2-((benzyloxy)methyl)-5-methoxy-5-methyltetrahydro-2H-pyran (0.520 g, 2.08 mmol) in methanol (10 mL) was added palladium on active carbon (0.500 g, 10 wt % purity) under a nitrogen atmosphere. The mixture was stirred at 25° C. under a hydrogen (15 Psi, balloon) atmosphere for 72 h. The reaction mixture was filtered over Celite. The filtrate was concentrated under reduced pressure to afford the title compound as a colorless oil (0.330 g, 2.06 mmol, 99% yield): ¹H NMR (400 MHz, CDCl₃) δ 3.71-3.60 (m, 2H), 3.58-3.48 (m, 1H), 3.47-3.39 (m, 1H), 3.34-3.28 (m, 1H), 3.26 (s, 3H), 2.03 (dd, J=4.4, 7.6 Hz, 1H), 1.99-1.89 (m, 1H), 1.67-1.53 (m, 2H), 1.51-1.37 (m, 1H), 1.29 (s, 3H).

### Step 4. Preparation of trans-5-methoxy-5-methyltetrahydro-2H-pyran-2-carboxylic acid

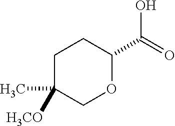

To a mixture of (trans-5-methoxy-5-methyltetrahydro-2H-pyran-2-yl)methanol (0.180 g, 1.12 mmol), sodium bromide(0.0231 g, 0.224 mmol) and saturated sodium bicarbonate (2.2 mL) in acetone (7.5 mL) was added 2,2,6,6-tetramethyl-piperidine-N-oxyl (0.0353 g, 0.224 mmol) and 1,3,5-trichloro-1,3,5-triazinane-2,4,6-trione (0.522 g, 2.25 mmol) under a nitrogen atmosphere at 0° C. The mixture was stirred at 25° C. for 12 h. The reaction mixture was quenched with isopropyl alcohol (1 mL). After filtration through a pad of Celite, the filter cake was washed with dichloromethane (30 mL). The filtrate was washed with hydrochloric acid (1 M, 2 mL) and brine (3×30 mL). The resulting solution was dried over sodium sulfate, filtered, and concentrated under reduced pressure to afford the title compound as a yellow oil (0.120 g, 61% yield): ¹H NMR (400 MHz, DMSO-d₆) δ 12.69 (s, 1H), 4.06-3.95 (m, 1H), 3.53 (d, J=11.2 Hz, 1H), 3.12 (s, 3H), 1.98-1.85 (m, 1H), 1.70-1.56 (m, 3H), 1.35-1.23 (m, 1H), 1.11 (s, 3H).

### Step 5. Preparation of rac-trans-N-(2-(4,4-difluorocyclohexyl)-4-(2,5-difluorophenyl)pyridin-3-yl)-5-methoxy-5-methyltetrahydro-2H-pyran-2-carboxamide

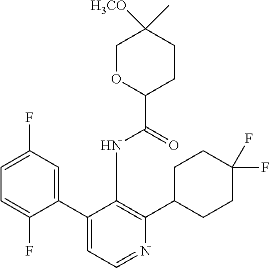 [SMILES: COC1(C)CCC(C(=O)Nc2c(-c3cc(F)ccc3F)ccnc2C2CCC(F)(F)CC2)OC1]

In a similar manner as described in other examples disclosed herein (e.g., Example 186, step 1), utilizing the appropriately substituted starting materials and intermediates, the title compound was afforded as a colorless solid (0.150 g, 53% yield): ¹H NMR (400 MHz, CDCl₃) δ 8.62 (d, J=4.8 Hz, 1H), 8.01 (s, 1H), 7.17-7.14 (m, 1H), 7.14-7.08 (m, 2H), 7.06-7.01 (m, 1H), 3.82 (dd, J=11.4, 2.8 Hz, 1H), 3.63 (dd, J=10.8, 2.0 Hz, 1H), 3.33 (d, J=10.8 Hz, 1H), 3.25 (s, 3H), 2.96 (t, J=12.0, 11.2 Hz, 1H), 2.31-2.19 (m, 2H), 2.17-2.07 (m, 2H), 2.03-1.93 (m, 2H), 1.92-1.84 (m, 3H), 1.83-1.73 (m, 1H), 1.61-1.53 (m, 1H), 1.21-1.19 (m, 3H), 1.18-1.06 (m, 1H).

### Step 6. Preparation of (trans)-N-(2-(4,4-difluorocyclohexyl)-4-(2,5-difluorophenyl)pyridin-3-yl)-5-methoxy-5-methyltetrahydro-2H-pyran-2-carboxamide P1 and P2/

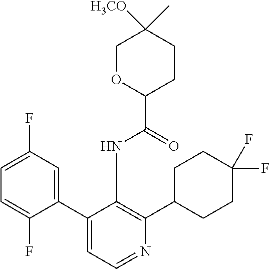 [SMILES: COC1(C)CCC(C(=O)Nc2c(-c3cc(F)ccc3F)ccnc2C2CCC(F)(F)CC2)OC1]

Trans-N-(2-(4,4-difluorocyclohexyl)-4-(2,5-difluorophenyl)pyridin-3-yl)-5-methoxy-5-methyltetrahydro-2H-pyran-2-carboxamide was purified by chiral SFC, eluting with a gradient of 5-40% of methanol containing 0.05% diethylamine in carbon dioxide (Column: Chiralcel OJ-3 50 mm×4.6 mm I.D., 3 μm). Two peaks were collected, dried under reduced pressure, and lyophilized.

Peak 1 (Retention Time=0.634 min): was obtained as a colorless solid (0.0143 g, 8.96% yield): ¹H NMR (400 MHz, CDCl₃) δ 8.62 (d, J=4.8 Hz, 1H), 8.00 (s, 1H), 7.18-7.09 (m, 3H), 7.06-7.01 (m, 1H), 3.82 (dd, J=11.2, 3.2 Hz, 1H), 3.63 (dd, J=10.8, 2.4 Hz, 1H), 3.33 (d, J=10.8 Hz, 1H), 3.25 (s, 3H), 2.96 (t, J=11.2 Hz, 1H), 2.33-2.19 (m, 2H), 2.18-2.04 (m, 2H), 2.03-1.93 (m, 2H), 1.92-1.84 (m, 3H), 1.80 (td, J=9.8, 4.4 Hz, 1H), 1.57-1.52 (m, 1H), 1.20 (s, 3H), 1.18-1.07 (m, 1H); MS (ES+) m/z 481.3 (M+1).
Peak 2 (Retention Time=0.725 min): was obtained as a white solid (0.0198 g, 13% yield); ¹H NMR (400 MHz, CDCl₃) δ 8.62 (d, J=4.8 Hz, 1H), 8.00 (s, 1H), 7.19-7.08 (m, 3H), 7.07-7.00 (m, 1H), 3.82 (dd, J=11.2, 2.8 Hz, 1H), 3.64 (dd, J=10.8, 2.4 Hz, 1H), 3.33 (d, J=11.2 Hz, 1H), 3.25 (s, 3H), 2.96 (t, J=11.6 Hz, 1H), 2.25 (dd, J=6.4, 3.2 Hz, 2H), 2.19-2.05 (m, 2H), 2.04-1.93 (m, 2H), 1.93-1.84 (m, 3H), 1.83-1.72 (m, 1H), 1.62-1.57 (m, 1H), 1.20 (s, 3H), 1.17-1.06 (m, 1H); MS (ES+) m/z 481.3 (M+1).

### Example 228

### Synthesis of N-(4-(4,4-difluorocyclohexyl)-2-(2,5-difluorophenyl)-5-fluoropyridin-3-yl)-2-ethoxypyrimidine-5-carboxamide

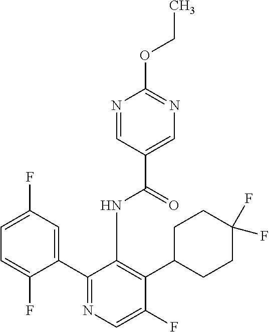 [SMILES: CCOc1ncc(C(=O)Nc2c(-c3cc(F)ccc3F)ncc(F)c2C2CCC(F)(F)CC2)cn1]

### Step 1. Preparation of 2-bromo-4-(4,4-difluorocyclohex-1-en-1-yl)-5-fluoro-3-nitropyridine

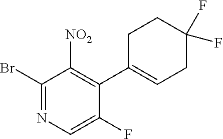 [SMILES: O=[N+]([O-])c1c(Br)ncc(F)c1C1=CCC(F)(F)CC1]

To a mixture of 2,4-dibromo-5-fluoro-3-nitropyridine (0.500 g, 1.67 mmol), 2-(4,4-difluorocyclohex-1-en-1-yl)-4,4,5,5-tetramethyl-1,3,2-dioxaborolane (0.406 g, 1.67 mmol) and potassium carbonate (0.691 g, 5.00 mmol) in dioxane (4.5 mL) and water (0.9 mL) was added [1,1′-bis(diphenylphosphino)ferrocene] dichloropalladium(II) (0.122 g, 0.166 mmol). The mixture was stirred at 65° C. under nitrogen atmosphere for 12 h. After being cooled to ambient temperature, the reaction mixture was concentrated under reduced pressure. The residue was purified by flash silica gel chromatography, eluting with 17% of ethyl acetate in petroleum ether, to afford the title compound as a yellow oil (0.150 g, 27% yield): ¹H NMR (400 MHz, CDCl₃) δ 8.42 (s, 1H), 5.80-5.69 (m, 1H), 2.77-2.63 (m, 2H), 2.62-2.53 (m, 2H), 2.17 (tt, J=13.6, 6.4 Hz, 2H).

### Step 2. Preparation of 4-(4,4-difluorocyclohex-1-en-1-yl)-2-(2,5-difluorophenyl)-5-fluoro-3-nitropyridine

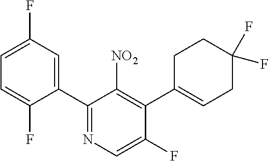 [SMILES: O=[N+]([O-])c1c(-c2cc(F)ccc2F)ncc(F)c1C1=CCC(F)(F)CC1]

To a mixture of 2-bromo-4-(4,4-difluorocyclohex-1-en-1-yl)-5-fluoro-3-nitropyridine (0.150 g, 0.444 mmol), (2,5-difluorophenyl)boronic acid (0.105 g, 0.667 mmol) and potassium carbonate (0.184 g, 1.33 mmol) in dioxane (1.5 mL) and water (0.3 mL) was added [1,1′-bis(diphenylphosphino)ferrocene]dichloropalladium(II) (0.0325 g, 0.0445 mmol under a nitrogen atmosphere. The mixture was stirred at 90° C. under a nitrogen atmosphere for 12 h. After being cooled to ambient temperature, the reaction mixture was concentrated under reduced pressure. The residue was purified by flash silica gel chromatography, eluting with 33% of ethyl acetate in petroleum ether, to afford the title compound as a yellow solid (0.120 g, 73% yield): ¹H NMR (400 MHz, CDCl₃) δ 8.75-8.72 (m, 1H), 7.28-7.24 (m, 1H), 7.21-7.08 (m, 2H), 5.68 (s, 1H), 2.72-2.64 (m, 4H), 2.24 (tt, J=13.6, 6.8 Hz, 2H).

### Step 3. Preparation of 4-(4,4-difluorocyclohexyl)-2-(2,5-difluorophenyl)-5-fluoropyridin-3-amine

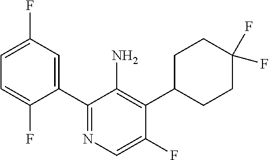 [SMILES: Nc1c(-c2cc(F)ccc2F)ncc(F)c1C1CCC(F)(F)CC1]

To a mixture of 4-(4,4-difluorocyclohex-1-en-1-yl)-2-(2,5-difluorophenyl)-5-fluoro-3-nitropyridine (0.120 g, 0.324 mmol) in methanol (5 mL) was added palladium on active carbon (0.100 g, 10 wt % purity) under a nitrogen atmosphere. The mixture was stirred at 25° C. under hydrogen (15 Psi, balloon) atmosphere for 12 h. The reaction mixture was filtered over Celite and the filtrate was concentrated under reduced pressure to afford the title compound as a colorless solid (0.100 g, 90% yield): ¹H NMR (400 MHz, CDCl₃) δ 7.97 (d, J=2.0 Hz, 1H), 7.21-7.09 (m, 3H), 3.82 (s, 2H), 2.82-2.70 (m, 1H), 2.34-2.23 (m, 4H), 1.94-1.75 (m, 4H).

### Step 4. Preparation of 2-chloro-N-(4-(4,4-difluorocyclohexyl)-2-(2,5-difluorophenyl)-5-fluoropyridin-3-yl)pyrimidine-5-carboxamide

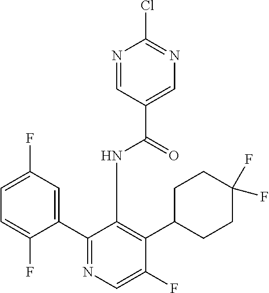 [SMILES: O=C(Nc1c(-c2cc(F)ccc2F)ncc(F)c1C1CCC(F)(F)CC1)c1cnc(Cl)nc1]

In a similar manner as described in other examples disclosed herein (e.g., Example 186, step 1), utilizing the appropriately substituted starting materials and intermediates, the title compound was afforded as a yellow oil (0.045 g, 32% yield): ¹H NMR (400 MHz, CDCl₃) δ 8.93 (s, 2H), 8.58 (d, J=1.6 Hz, 1H), 7.85 (d, J=5.2 Hz, 1H), 7.25 (d, J=1.6 Hz, 1H), 7.16-7.09 (m, 2H), 2.81 (t, J=12.4 Hz, 1H), 2.39-2.17 (m, 4H), 1.98 (d, J=12.4 Hz, 2H), 1.83-1.63 (m, 2H).

### Step 5. Preparation of N-(4-(4,4-difluorocyclohexyl)-2-(2,5-difluorophenyl)-5-fluoropyridin-3-yl)-2-ethoxypyrimidine-5-carboxamide

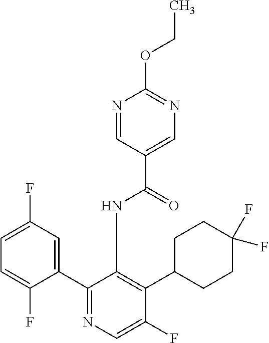 [SMILES: CCOc1ncc(C(=O)Nc2c(-c3cc(F)ccc3F)ncc(F)c2C2CCC(F)(F)CC2)cn1]

To a mixture of 2-chloro-N-(4-(4,4-difluorocyclohexyl)-2-(2,5-difluorophenyl)-5-fluoropyridin-3-yl) pyrimidine-5-carboxamide (0.0300 g, 0.0621 mmol) in ethanol (2 mL) was added cesium carbonate (0.0607 g, 0.186 mmol). The mixture was stirred at 80° C. for 12 h. After being cooled to room temperature, the reaction mixture was concentrated under reduced pressure. The residue was purified by preparative HPLC, eluting with a gradient of 45-75% of acetonitrile in water containing 0.1% formic acid, to afford the title compound as a colorless solid (0.0183 g, 38% yield, 95% purity): ¹H NMR (400 MHz, CDCl₃) δ 8.84 (s, 2H), 8.55 (d, J=1.6 Hz, 1H), 7.69 (d, J=4.8 Hz, 1H), 7.26-7.22 (m, 1H), 7.17-7.05 (m, 2H), 4.52 (q, J=7.2 Hz, 2H), 2.84 (t, J=12.4 Hz, 1H), 2.36-2.18 (m, 4H), 2.01-1.98 (m, 2H), 1.82-1.62 (m, 2H), 1.46 (t, J=7.2 Hz, 3H); (ES+) m/z 493.2 (M+1).

### Example 229

### Synthesis of N-(2-(4,4-difluorocyclohexyl)-4-(1H-pyrazol-3-yl)pyridin-3-yl)-2-isopropylpyrimidine-5-carboxamide

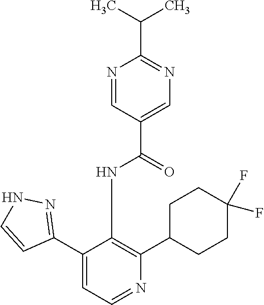 [SMILES: CC(C)c1ncc(C(=O)Nc2c(-c3cc[nH]n3)ccnc2C2CCC(F)(F)CC2)cn1]

### Step 1. Preparation of 2-chloro-3-nitro-4-(1H-pyrazol-3-yl)pyridine

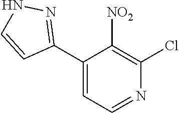 [SMILES: O=[N+]([O-])c1c(-c2cc[nH]n2)ccnc1Cl]

A mixture of 2,4-dichloro-3-nitropyridine (5.00 g, 25.9 mmol), 3-(4,4,5,5-tetramethyl-1,3,2-dioxaborolan-2-yl)-1H-pyrazole (5.03 g, 25.9 mmol), potassium carbonate (10.7 g, 77.7 mmol) and [1,1′-bis(diphenylphosphino)ferrocene]dichloropalladium(II) (1.90 g, 2.59 mmol) in dioxane (40 mL) and water (10 mL) was stirred at 60° C. for 12 h under a nitrogen atmosphere. The mixture was poured into saturated aqueous sodium bicarbonate (50 mL). The mixture was extracted with ethyl acetate (3×50 mL). The combined organic layers were washed with brine (50 mL), dried over sodium sulfate, filtered and the filtrate was evaporated under reduced pressure. The residue was purified by flash silica gel chromatography, eluting with a gradient of 0-38% of ethyl acetate in petroleum ether, to afford the title compound as a yellow solid (2.49 g, 37% yield, 86% purity); ¹H NMR (400 MHz, DMSO-d₆) δ 13.60 (s, 1H), 8.63 (d, J=5.2 Hz, 1H), 8.08 (d, J=5.2 Hz, 1H), 7.97 (d, J=2.4 Hz, 1H), 6.99 (d, J=1.2 Hz, 1H).

### Step 2. Preparation of 2-chloro-3-nitro-4-(1-(tetrahydro-2H-pyran-2-yl)-1H-pyrazol-3-yl)pyridine

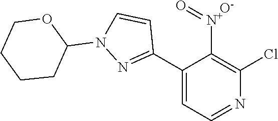

To a solution of 2-chloro-3-nitro-4-(1H-pyrazol-3-yl)pyridine (2.39 g, 10.6 mmol) and 4-methylbenzene-sulfonic acid (0.184 g, 1.07 mmol) in tetrahydrofuran (30 mL) was added 3,4-dihydro-2H-pyran (2.69 g, 31.9 mmol) at 25° C. The mixture was stirred at 70° C. for 12 h. After cooling to ambient temperature, the mixture was concentrated under reduced pressure. The residue was purified by flash silica gel chromatography, eluting with a gradient of 0-35% of ethyl acetate in petroleum ether, to afford the title compound as a yellow solid (1.53 g, 42% yield): ¹H NMR (400 MHz, DMSO-d₆) δ 8.66 (d, J=5.4 Hz, 1H), 8.20-7.99 (m, 2H), 7.03 (d, J=2.4 Hz, 1H), 5.50 (dd, J=8.8, 2.4 Hz, 1H), 3.91-3.80 (m, 1H), 3.70-3.58 (m, 1H), 1.92 (td, J=8.4, 3.8 Hz, 2H), 1.71-1.62 (m, 1H), 1.61-1.47 (m, 3H).

### Step 3. Preparation of 2-(4,4-difluorocyclohex-1-en-1-yl)-3-nitro-4-(1-(tetrahydro-2H-pyran-2-yl)-1H-pyrazol-3-yl)pyridine

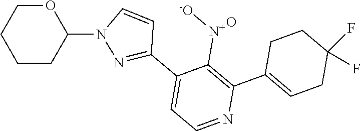

To a solution of 2-chloro-3-nitro-4-(1-(tetrahydro-2H-pyran-2-yl)-1H-pyrazol-3-yl)pyridine (0.500 g, 1.62 mmol), 2-(4,4-difluorocyclohex-1-en-1-yl)-4,4,5,5-tetramethyl-1,3,2-dioxa-borolane (0.435 g, 1.78 mmol) and potassium carbonate (0.336 g, 2.43 mmol) in dioxane (12 mL) and water (4 mL) was added [1,1′-bis(diphenylphosphino)ferrocene]dichloropalladium(II) (0.119 g, 0.163 mmol) at 25° C. under a nitrogen atmosphere. The mixture was stirred at 100° C. for 12 h. After cooling to ambient temperature, the mixture was concentrated under reduced pressure. The residue was purified by flash silica gel chromatography, eluting with a gradient of 0-30% of ethyl acetate in petroleum ether, to afford the title compound as a yellow solid (0.735 g, crude): ¹H NMR (400 MHz, DMSO-d₆) δ 8.76 (d, J=5.2 Hz, 1H), 8.06 (d, J=2.4 Hz, 1H), 7.88 (d, J=5.2 Hz, 1H), 6.84 (d, J=2.4 Hz, 1H), 5.76 (s, 1H), 5.50 (dd, J=8.4, 2.4 Hz, 1H), 3.90-3.81 (m, 1H), 3.70-3.58 (m, 1H), 2.76-2.60 (m, 4H), 2.17 (dt, J=13.8, 7.2 Hz, 2H), 2.08-1.88 (m, 3H), 1.73-1.61 (m, 1H), 1.59-1.48 (m, 2H).

### Step 4. Preparation of 2-(4,4-difluorocyclohexyl)-4-(1-(tetrahydro-2H-pyran-2-yl)-1H-pyrazol-3-yl)pyridin-3-amine

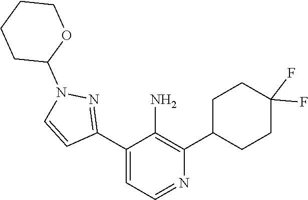 [SMILES: Nc1c(-c2ccn(C3CCCCO3)n2)ccnc1C1CCC(F)(F)CC1]

To a solution of 2-(4,4-difluorocyclohex-1-en-1-yl)-3-nitro-4-(1-(tetrahydro-2H-pyran-2-yl)-1H-pyrazol-3-yl)pyridine (0.180 g, 0.461 mmol) in methanol (5 mL) was added palladium on carbon (0.0350 g, 10 wt %) under nitrogen atmosphere. The suspension was degassed and purged with hydrogen three times. The mixture was stirred under a hydrogen atmosphere (15 Psi) at 25° C. for 12 h. The mixture was filtered and the filtrate was concentrated under reduced pressure to afford the title compound as a colorless oil (0.159 g, 90% yield): ¹H NMR (400 MHz, DMSO-d₆) δ 8.02 (d, J=2.4 Hz, 1H), 7.77 (d, J=5.2 Hz, 1H), 7.38 (d, J=5.2 Hz, 1H), 6.93 (d, J=2.4 Hz, 1H), 6.42 (s, 2H), 5.51 (dd, J=9.6, 2.4, Hz, 1H), 3.95 (s, 1H), 3.72-3.63 (m, 1H), 3.11-3.02 (m, 1H), 2.18-2.08 (m, 3H), 2.00 (s, 1H), 1.96 (dd, J=8.8, 3.2 Hz, 2H), 1.92-1.65 (m, 6H), 1.57 (dd, J=7.2, 3.6 Hz, 2H).

### Step 5. Preparation of N-(2-(4,4-difluorocyclohexyl)-4-(1-(tetrahydro-2H-pyran-2-yl)-1H-pyrazol-3-yl)pyridin-3-yl)-2-isopropylpyrimidine-5-carboxamide

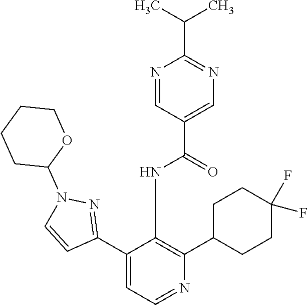 [SMILES: CC(C)c1ncc(C(=O)Nc2c(-c3ccn(C4CCCCO4)n3)ccnc2C2CCC(F)(F)CC2)cn1]

A mixture of 2-isopropylpyrimidine-5-carboxylic acid (0.110 g, 0.662 mmol), 2-chloro-1-methylpyridinium iodide (0.450 g, 1.76 mmol) and N-ethyl-N-isopropylpropan-2-amine (0.227 g, 1.76 mmol) in tetrahydrofuran (5 mL) was stirred at 25° C. for 0.5 h. To the mixture was added 2-(4,4-difluoro-cyclohexyl)-4-(1-(tetrahydro-2H-pyran-2-yl)-1H-pyrazol-3-yl)pyridin-3-amine (0.159 g, 0.439 mmol). The mixture was stirred at 65° C. for 12 h. After cooling to ambient temperature, the mixture was concentrated under reduced pressure. The residue was purified by flash silica gel chromatography, eluting with a gradient of 0-40% of ethyl acetate in petroleum ether, followed by preparative HPLC, eluting with a gradient of 40-70% of 0.225% formic acid in water, to afford the title compound as a colorless solid, (0.160 g, 39% yield, 54% purity); ¹H NMR (400 MHz, CDCl₃) δ 10.81 (d, J=1.2 Hz, 1H), 9.36 (s, 2H), 8.64 (d, J=5.2 Hz, 1H), 7.70 (d, J=2.4 Hz, 1H), 7.58 (s, 1H), 6.78 (s, 1H), 5.35 (dd, J=9.6, 2.4 Hz, 1H), 4.16-4.06 (m, 1H), 3.72 (dt, J=11.4, 2.4 Hz, 1H), 3.34 (q, J=6.8 Hz, 1H), 3.03 (t, J=11.2 Hz, 1H), 2.17-1.91 (m, 7H), 1.88-1.53 (m, 7H), 1.40 (d, J=6.8 Hz, 6H).

### Step 6. Preparation of N-(2-(4,4-difluorocyclohexyl)-4-(1H-pyrazol-3-yl)pyridin-3-yl)-2-isopropylpyrimidine-5-carboxamide

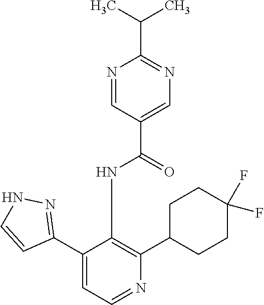 [SMILES: CC(C)c1ncc(C(=O)Nc2c(-c3cc[nH]n3)ccnc2C2CCC(F)(F)CC2)cn1]

To a solution of N-(2-(4,4-difluorocyclohexyl)-4-(1-(tetrahydro-2H-pyran-2-yl)-1H-pyrazol-3-yl)pyridin-3-yl)-2-isopropylpyrimidine-5-carboxamide (0.050 g, 0.0979 mmol) in dichloromethane (1 mL) was added hydrogen chloride/dioxane (4 M, 1 mL) at 25° C. The mixture was stirred at 25° C. for 1 h. The mixture was concentrated under reduced pressure. The residue was purified by preparative HPLC, eluting with a gradient of 25-55% of acetonitrile in 0.1% of ammonium hydroxide in water. The residue was diluted with ethyl acetate (20 mL) and washed with water (3×20 mL), dried over sodium sulfate, filtered and concentrated under reduced pressure to afford the title compound as a colorless solid (0.0163 g, 39% yield): ¹H NMR (400 MHz, DMSO-d₆) δ 13.20 (s, 1H), 10.39 (s, 1H), 9.24 (s, 2H), 8.53 (d, J=5.0 Hz, 1H), 7.79 (s, 1H), 7.69 (d, J=5.0 Hz, 1H), 6.68 (s, 1H), 3.25 (dt, J=13.8, 6.8 Hz, 1H), 3.20-3.09 (m, 1H), 2.13-1.95 (m, 3H), 1.93-1.75 (m, 5H), 1.33 (d, J=6.8 Hz, 6H); MS (ES+) m/z 427.3 (M+1).

### Example 230

### Synthesis of N-(2-(4,4-difluorocyclohexyl)-4-(1H-pyrazol-3-yl)pyridin-3-yl)-4-(2-(trifluoromethyl)oxetan-2-yl)benzamide

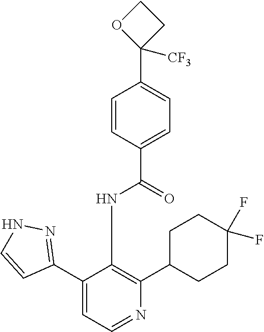 [SMILES: O=C(Nc1c(-c2cc[nH]n2)ccnc1C1CCC(F)(F)CC1)c1ccc(C2(C(F)(F)F)CCO2)cc1]

### Step 1. Preparation of N-(2-(4,4-difluorocyclohexyl)-4-(1-(tetrahydro-2H-pyran-2-yl)-1H-pyrazol-3-yl)pyridin-3-yl)-4-(2,2,2-trifluoroacetyl)benzamide

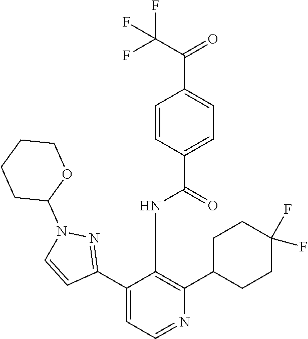 [SMILES: O=C(Nc1c(-c2ccn(C3CCCCO3)n2)ccnc1C1CCC(F)(F)CC1)c1ccc(C(=O)C(F)(F)F)cc1]

In a similar manner as described in other examples disclosed herein (e.g., Example 129, step 5), utilizing the appropriately substituted starting materials and intermediates, the title compound was afforded as a light-yellow solid (0.490 g, 60% yield): ¹H NMR (400 MHz, CDCl₃) δ 10.52-10.31 (m, 1H), 8.55 (dd, J=9.2, 5.2, Hz, 1H), 8.22 (s, 2H), 8.02 (d, J=8.4 Hz, 1H), 7.83 (d, J=8.4 Hz, 1H), 7.65 (dd, J=2.4, 1.2 Hz, 1H), 7.40 (d, J=5.2 Hz, 1H), 6.70 (dd, J=4.0, 2.4 Hz, 1H), 5.25 (d, J=9.2 Hz, 1H), 4.19 (br s, 1H), 4.07-3.96 (m, 1H), 3.71-3.57 (m, 1H), 3.06-2.94 (m, 1H), 2.30-2.06 (m, 6H), 1.97-1.70 (m, 7H); MS (ES+) m/z 581.3 (M+1+18).

### Step 2. Preparation of N-(2-(4,4-difluorocyclohexyl)-4-(1-(tetrahydro-2H-pyran-2-yl)-1H-pyrazol-3-yl)pyridin-3-yl)-4-(2-(trifluoromethyl)oxetan-2-yl)benzamide

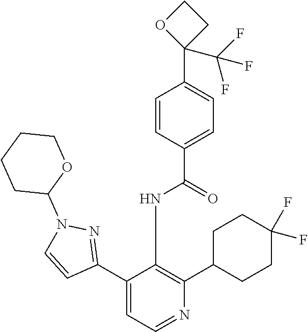 [SMILES: O=C(Nc1c(-c2ccn(C3CCCCO3)n2)ccnc1C1CCC(F)(F)CC1)c1ccc(C2(C(F)(F)F)CCO2)cc1]

To a mixture of potassium 2-methylpropan-2-olate (0.335 g, 2.99 mmol) in dimethylsulfoxide (9.6 mL) was added trimethylsulfonium iodide (0.609 g, 2.99 mmol) in one portion at 25° C. The mixture was stirred at 25° C. for 10 min, then a solution of N-(2-(4,4-difluorocyclohexyl)-4-(1-(tetrahydro-2H-pyran-2-yl)-1H-pyrazol-3-yl)pyridin-3-yl)-4-(2,2,2-trifluoroacetyl)benzamide (0.480 g, 0.853 mmol) in dimethyl-sulfoxide (4.8 mL) was added. The mixture was stirred at 25° C. for 4 h. The mixture was added to saturated ammonium chloride (80 mL), extracted with ethyl acetate (3×80 mL) and concentrated. The residue was purified by preparative HPLC, eluting with a gradient of 50-77% of acetonitrile in water containing ammonium bicarbonate, to afford the title compound as a colorless solid (0.255 g, 50% yield): ¹H NMR (400 MHz, CDCl₃) δ 10.37 (s, 1H), 8.55 (d, J=5.2 Hz, 1H), 8.12 (d, J=8.4 Hz, 2H), 7.67-7.53 (m, 3H), 7.39 (d, J=5.2 Hz, 1H), 6.69 (d, J=2.4 Hz, 1H), 5.32-5.20 (m, 1H), 4.95-4.80 (m, 1H), 4.63 (td, J=6.0, 9.2 Hz, 1H), 4.00 (d, J=11.6 Hz, 1H), 3.72-3.53 (m, 1H), 3.35 (ddd, J=11.8, 8.8, 6.4 Hz, 1H), 3.10-2.87 (m, 2H), 2.33-2.00 (m, 7H), 1.99-1.69 (m, 6H); MS (ES+) m/z 591.1 (M+1).

### Step 3. Preparation of N-(2-(4,4-difluorocyclohexyl)-4-(1H-pyrazol-3-yl)pyridin-3-yl)-4-(2-(trifluoromethyl)oxetan-2-yl)benzamide

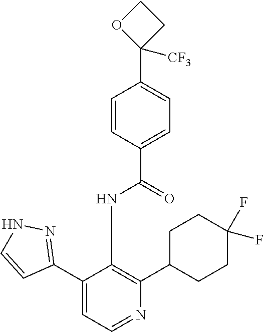 [SMILES: O=C(Nc1c(-c2cc[nH]n2)ccnc1C1CCC(F)(F)CC1)c1ccc(C2(C(F)(F)F)CCO2)cc1]

To a mixture of N-(2-(4,4-difluorocyclohexyl)-4-(1-(tetrahydro-2H-pyran-2-yl)-1H-pyrazol-3-yl) pyridin-3-yl)-4-(2-(trifluoromethyl) oxetan-2-yl)benzamide (0.0500 g, 0.0846 mmol) in dichloromethane (1 mL) was added trifluoroacetic acid (0.308 g, 2.70 mmol). The mixture was stirred at 25° C. for 2 h. The residue was poured into saturated sodium bicarbonate (5 mL) and extracted with ethyl acetate (3×15 mL).

The combined organic extracts were dried over anhydrous sodium sulfate, filtered, and the filtrate was concentrated in vacuo. The residue was purified by flash silica gel chromatography, eluting with 50% of ethyl acetate in petroleum ether, to afford the title compound as a colorless solid (0.0127 g, 28% yield): ¹H NMR (400 MHz, DMSO-d₆) δ 13.19 (s, 1H), 10.26 (s, 1H), 8.61-8.46 (m, 1H), 8.09 (d, J=8.2 Hz, 2H), 7.78 (s, 1H), 7.70 (d, J=5.0 Hz, 1H), 7.58 (d, J=8.2 Hz, 2H), 6.62 (s, 1H), 4.82-4.72 (m, 1H), 4.58 (td, J=6.0, 9.2 Hz, 1H), 3.30-3.25 (m, 1H), 3.15-2.97 (m, 2H), 2.14-2.03 (m, 2H), 1.94-1.75 (m, 6H); MS (ES+) m/z 507.3 (M+1).

### Example 231

### Synthesis of N-(2-(4,4-difluorocyclohexyl)-4-(1H-pyrazol-3-yl)pyridin-3-yl)-2-isopropylpyrimidine-5-carboxamide

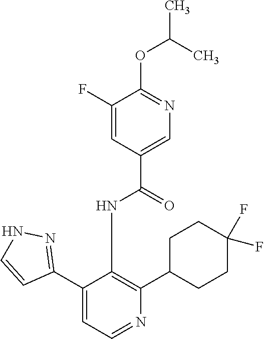 [SMILES: CC(C)Oc1ncc(C(=O)Nc2c(-c3cc[nH]n3)ccnc2C2CCC(F)(F)CC2)cc1F]

### Step 1. Preparation of N-(2-(4,4-difluorocyclohexyl)-4-(1-(tetrahydro-2H-pyran-2-yl)-1H-pyrazol-3-yl)pyridin-3-yl)-5,6-difluoronicotinamide

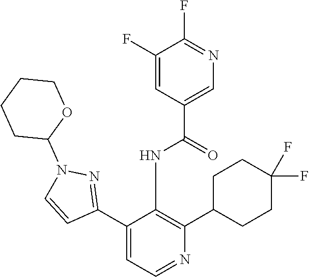 [SMILES: O=C(Nc1c(-c2ccn(C3CCCCO3)n2)ccnc1C1CCC(F)(F)CC1)c1cnc(F)c(F)c1]

In a similar manner as described in Example 129 step 5, utilizing the appropriately substituted starting materials and intermediates, the title compound was afforded as a light-yellow solid (0.519 g, 82% yield): ¹H NMR (400 MHz, DMSO-d₆) δ 10.37 (s, 1H), 8.71 (s, 1H), 8.59-8.47 (m, 2H), 7.96 (d, J=2.4 Hz, 1H), 7.68 (d, J=5.0 Hz, 1H), 6.76 (d, J=2.4 Hz, 1H), 5.33 (dd, J=2.2, 10.0 Hz, 1H), 3.88 (d, J=11.4 Hz, 1H), 3.56 (dt, J=2.6, 11.4 Hz, 1H), 3.20-3.10 (m, 1H), 2.09 (d, J=4.8 Hz, 2H), 1.98-1.72 (m, 9H), 1.63-1.36 (m, 3H).

### Step 2. Preparation of N-(2-(4,4-difluorocyclohexyl)-4-(1-(tetra-hydro-2H-pyran-2-yl)-1H-pyrazol-3-yl)pyridin-3-yl)-5-fluoro-6-isopropoxynicotinamide

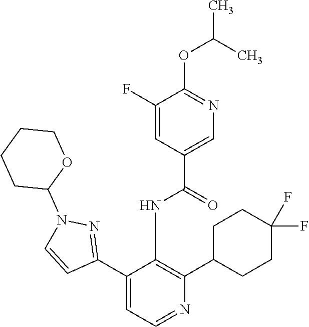 [SMILES: CC(C)Oc1ncc(C(=O)Nc2c(-c3ccn(C4CCCCO4)n3)ccnc2C2CCC(F)(F)CC2)cc1F]

To a solution of N-(2-(4,4-difluorocyclohexyl)-4-(1-(tetrahydro-2H-pyran-2-yl)-1H-pyrazol-3-yl)pyridin-3-yl)-5,6-difluoronicotinamide (0.100 g, 0.198 mmol) in dioxane (1.5 mL) was added sodium propan-2-olate (0.0815 g, 0.993 mmol) in one portion at 20° C. The mixture was stirred at 20° C. for 12 h. The mixture was poured into saturated aqueous sodium bicarbonate (20 mL) and extracted with ethyl acetate (3×30 mL). The combined organic layers were washed with brine (10 mL), dried over anhydrous sodium sulfate, filtered and the filtrate was evaporated under reduced pressure. The residue was purified by flash silica gel chromatography, eluting with 66% of ethyl acetate in petroleum ether, to afford the title compound as a colorless solid (0.130 g): ¹H NMR (400 MHz, CDCl₃) δ 10.38 (s, 1H), 8.71 (d, J=2.0 Hz, 1H), 8.55 (d, J=5.0 Hz, 1H), 8.04-7.89 (m, 1H), 7.67 (d, J=2.6 Hz, 1H), 7.39 (d, J=5.0 Hz, 1H), 6.70 (d, J=2.6 Hz, 1H), 5.64-5.47 (m, 1H), 5.35 (dd, J=9.8, 2.2 Hz, 1H), 4.17-4.01 (m, 1H), 3.82-3.66 (m, 1H), 3.09-2.94 (m, 1H), 2.24 (d, J=3.0 Hz, 2H), 2.16-1.76 (m, 9H), 1.73-1.58 (m, 3H), 1.46 (d, J=6.2 Hz, 6H).

### Step 3. Preparation of N-(2-(4,4-difluorocyclohexyl)-4-(1H-pyrazol-3-yl)pyridin-3-yl)-2-isopropylpyrimidine-5-carboxamide

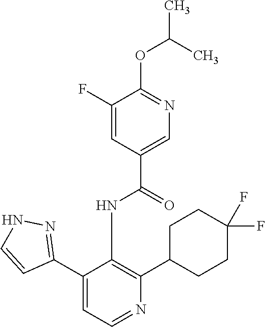 [SMILES: CC(C)Oc1ncc(C(=O)Nc2c(-c3cc[nH]n3)ccnc2C2CCC(F)(F)CC2)cc1F]

To a solution of N-(2-(4,4-difluorocyclohexyl)-4-(1-(tetrahydro-2H-pyran-2-yl)-1H-pyrazol-3-yl)pyridin-3-yl)-5-fluoro-6-isopropoxynicotinamide (0.130 g, 0.239 mmol) in dioxane (4 mL) was added hydrogen chloride in dioxane (4.0 M, 4.0 mL) dropwise at 20° C. The mixture was stirred at 20° C. for 1 h. The mixture was poured into saturated aqueous sodium carbonate (20 mL) and extracted with ethyl acetate (3×20 mL). The combined organic layers were washed with brine (10 mL), dried over anhydrous sodium sulfate, filtered and the filtrate was evaporated under reduced pressure. The residue was purified by preparative HPLC, eluting with a gradient of 34-64% of acetonitrile in water containing 0.225% formic acid, to afford the title compound as a colorless solid (0.0331 g, 30% yield): ¹H NMR (400 MHz, CDCl₃) δ 10.36 (s, 1H), 8.66 (d, J=1.0 Hz, 1H), 8.56 (d, J=4.8 Hz, 1H), 8.01-7.90 (m, 1H), 7.67 (d, J=1.8 Hz, 1H), 7.43 (d, J=4.8 Hz, 1H), 6.75 (s, 1H), 5.53-5.42 (m, 1H), 2.98 (t, J=10.8 Hz, 1H), 2.29-2.17 (m, 2H), 2.17-1.97 (m, 4H), 1.90-1.69 (m, 2H), 1.44 (d, J=6.0 Hz, 6H); MS (ES+) m/z 460.3 (M+1).

### Example 232

In a similar manner as described in Example 231, utilizing the appropriately substituted starting materials and intermediates, the following compounds were prepared:

| Example | Structure | | MS (ES+) | |
|---|---|---|---|---|
| No. | Name | Yield | m/z | ¹H-NMR |
| 232 | 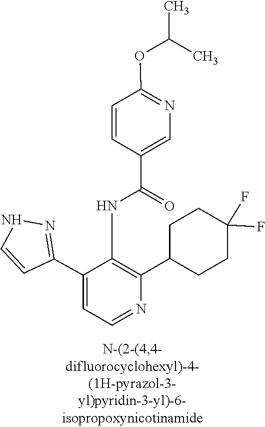 [SMILES: CC(C)Oc1ccc(C(=O)Nc2c(-c3cc[nH]n3)ccnc2C2CCC(F)(F)CC2)cn1] | 16% | 442.3 (M + 1) | ¹H NMR (400 MHz, DMSO-d₆) δ 13.20 (s, 1H), 10.10 (s, 1H), 8.82 (d, J = 2.0 Hz, 1H), 8.51 (d, J = 5.2 Hz, 1H), 8.21 (dd, J = 8.4, 2.4 Hz, 1H), 7.80-7.63 (m, 2H), 6.88 (d, J = 8.4 Hz, 1H), 6.61 (d, J = 2.0 Hz, 1H), 5.36 (td, J = 6.4, 12.4 Hz, 1H), 3.15-3.07 (m, 1H), 2.17-2.02 (m, 2H), 1.97-1.79 (m, 6H), 1.34 (d, J = 8 Hz, 6H) |

### Example 233

### Synthesis of N-(2-(4,4-difluorocyclohexyl)-4-(1H-pyrazol-3-yl)pyridin-3-yl)-2-isopropylpyrimidine-5-carboxamide

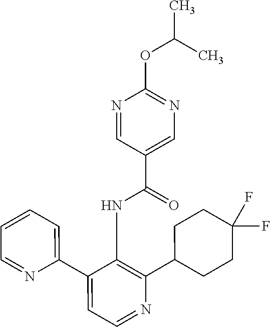 [SMILES: CC(C)Oc1ncc(C(=O)Nc2c(-c3ccccn3)ccnc2C2CCC(F)(F)CC2)cn1]

### Step 1. Preparation of tert-butyl (2′-chloro-[2,4′-bipyridin]-3′-yl)-carbamate

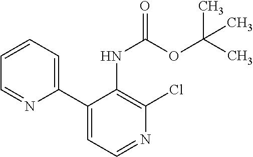 [SMILES: CC(C)(C)OC(=O)Nc1c(-c2ccccn2)ccnc1Cl]

A mixture of tert-butyl (2-chloro-4-iodopyridin-3-yl)carbamate (0.400 g, 1.13 mmol), pyridin-2-ylzinc(II) bromide (0.5 M, 5.64 mL) and tetrakis[triphenylphosphine]palladium(0) (0.130 g, 0.113 mmol) in tetrahydrofuran (5 mL) was stirred at 90° C. for 4 h under argon under microwave irradiation. After cooling to ambient temperature, four batches of this mixture were combined and added to saturated ammonium chloride (80 mL), extracted with ethyl acetate (3×50 mL), dried and filtered. The filtrate was concentrated, and the residue was purified by flash silica gel chromatography, eluting with a gradient 33% of ethyl acetate in petroleum ether, followed by flash silica gel chromatography, eluting with dichloromethane, to afford the title compound as a light-yellow solid: ¹H NMR (400 MHz, CDCl₃) δ 8.73 (d, J=4.8 Hz, 1H), 8.32 (d, J=5.2 Hz, 1H), 7.85 (dt, J=7.6, 1.6 Hz, 1H), 7.78 (br s, 1H), 7.68 (d, J=8.0 Hz, 1H), 7.45 (d, J=5.2 Hz, 1H), 7.36 (ddd, J=7.6, 4.8, 0.8 Hz, 1H), 1.34 (s, 9H).

### Step 2. Preparation of tert-butyl (2′-(4,4-difluorocyclohex-1-en-1-yl)-[2,4′-bipyridin]-3′-yl)carbamate

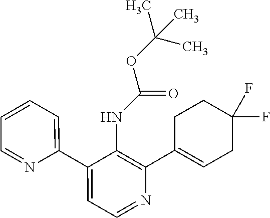 [SMILES: CC(C)(C)OC(=O)Nc1c(-c2ccccn2)ccnc1C1=CCC(F)(F)CC1]

To a mixture of tert-butyl (2′-chloro-[2,4′-bipyridin]-3′-yl)carbamate (0.185 g, 0.605 mmol), 2-(4,4-difluorocyclohex-1-en-1-yl)-4,4,5,5-tetramethyl-1,3,2-dioxaborolane (0.295 g, 1.21 mmol), potassium carbonate (0.502 g, 3.63 mmol) and 1,1-bis(diphenylphosphino)ferrocene-palladium(ii) dichloride dichloromethane complex (0.0988 g, 0.121 mmol) was added 1,4-dioxane (4 mL) and water (1 mL). The mixture was de-gassed and then heated to 100° C. for 12 hours under a nitrogen atmosphere. After cooling to ambient temperature, the combined mixture (3 batches) was diluted with water (20 mL) and extracted with dichloromethane (5×20 mL). The organic layer was dried over sodium sulfate, filtered, and concentrate. The residue was purified by flash silica gel chromatography, eluting with 14% of ethyl containing 0.25% triethylamine in dichloromethane, to afford the title compound as a light-yellow solid (0.600 g, 83% yield): ¹H NMR (400 MHz, MeOD-d₄), 8.69 (d, J=4.4 Hz, 1H), 8.50 (d, J=5.2 Hz, 1H), 7.94 (dt, J=7.6, 1.6 Hz, 1H), 7.71 (d, J=7.6 Hz, 1H), 7.53 (d, J=5.2 Hz, 1H), 7.50-7.43 (m, 1H), 5.83 (s, 1H), 2.82-2.57 (m, 4H), 2.18 (tt, J=13.6, 6.8 Hz, 2H), 1.36 (s, 9H).

### Step 3. Preparation of tert-butyl (2′-(4,4-difluorocyclohexyl)-[2,4′-bipyridin]-3′-yl)carbamate

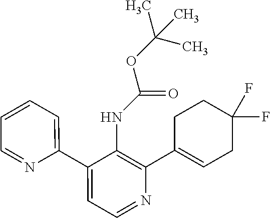 [SMILES: CC(C)(C)OC(=O)Nc1c(-c2ccccn2)ccnc1C1=CCC(F)(F)CC1]

To a solution of tert-butyl (2′-(4,4-difluorocyclohex-1-en-1-yl)-[2,4′-bipyridin]-3′-yl)carbamate (0.550 g, 1.42 mmol) in methanol (100 mL) was added palladium on active carbon (0.275 g, 10 wt % purity) under a nitrogen atmosphere. The suspension was degassed under vacuum and purged with hydrogen several times. The mixture was stirred under hydrogen (15 psi) at 25° C. for 3 h. The mixture was then filtered over Celite. The filtrate was concentrated to afford the title compound as a light-yellow solid (0.410 g, 74% yield): ¹H NMR (400 MHz, MeOD-d₄) δ 8.67 (d, J=4.8 Hz, 1H), 8.52 (d, J=4.8 Hz, 1H), 7.93 (dt, J=7.6, 1.6 Hz, 1H), 7.66 (d, J=6.6 Hz, 1H), 7.50-7.40 (m, 2H), 3.28-3.17 (m, 1H), 2.25-2.12 (m, 2H), 2.10-1.77 (m, 6H), 1.33 (s, 9H).

### Step 4. Preparation of 2′-(4,4-difluorocyclohexyl)-[2,4′-bipyridin]-3′-amine

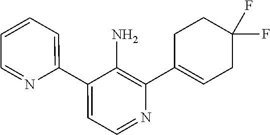 [SMILES: Nc1c(-c2ccccn2)ccnc1C1=CCC(F)(F)CC1]

To a solution of tert-butyl (2′-(4,4-difluorocyclohexyl)-[2,4′-bipyridin]-3′-yl)carbamate (1.20 g, 3.08 mmol) in dichloromethane (6 mL) was added trifluoroacetic acid (9.24 g, 81.0 mmol). The mixture was stirred at 25° C. for 2 h. The mixture was diluted with dichloromethane (200 mL) and concentrated in vacuo. This dilution and concentration cycle was repeated three times. The residue was dissolved in dichloromethane (100 mL) and adjusted to pH=8 with saturated sodium bicarbonate. The resulting mixture was extracted with dichloromethane (2×50 mL), dried over sodium sulfate, and filtered. The filtrate was concentrated under vacuum to afford the title compound as a light-yellow solid (0.800 g, crude): ¹H NMR (400 MHz, MeOD-d₄) S 8.69-8.63 (m, 1H), 7.98-7.90 (m, 1H), 7.88-7.79 (m, 2H), 7.39 (ddd, J=7.6, 4.8, 1.2 Hz, 1H), 7.36 (d, J=5.2 Hz, 1H), 3.10-3.01 (m, 1H), 2.20-2.14 (m, 2H), 2.00-1.90 (m, 6H).

### Step 5. Preparation of 2-chloro-N-(2′-(4,4-difluorocyclohexyl)-[2,4′-bipyridin]-3′-yl)pyrimidine-5-carboxamide

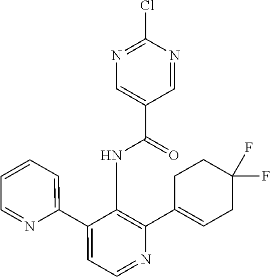 [SMILES: O=C(Nc1c(-c2ccccn2)ccnc1C1=CCC(F)(F)CC1)c1cnc(Cl)nc1]

In a similar manner as described in examples disclosed herein, utilizing the appropriately substituted starting materials and intermediates, the title compound was afforded as a light-yellow solid (0.580 g, 49% yield): ¹H NMR (400 MHz, CDCl₃) δ 11.67 (s, 1H), 9.18 (s, 2H), 8.68 (d, J=4.8 Hz, 2H), 7.92 (td, J=1.6, 7.6, 1.6 Hz, 1H), 7.79 (d, J=7.6 Hz, 1H), 7.46-7.34 (m, 2H), 3.01-2.89 (m, 1H), 2.32-1.99 (m, 6H), 1.91-1.79 (m, 2H); MS (ES+) m/z 430.1 (M+1).

### Step 6. Preparation of N-(2-(4,4-difluorocyclohexyl)-4-(1H-pyrazol-3-yl)pyridin-3-yl)-2-isopropylpyrimidine-5-carboxamide

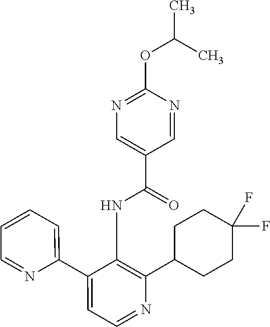 [SMILES: CC(C)Oc1ncc(C(=O)Nc2c(-c3ccccn3)ccnc2C2CCC(F)(F)CC2)cn1]

To a solution of 2-chloro-N-(2′-(4,4-difluorocyclohexyl)-[2,4′-bipyridin]-3′-yl)pyrimidine-5-carboxamide (0.200 g, 0.465 mmol) in propan-2-ol (8 mL) was added cesium carbonate (0.455 g, 1.40 mmol). The mixture was stirred at 80° C. for 12 h. After cooling to ambient temperature, the mixture was combined with two other batches of the same mixture. The resulting mixture was filtered and concentrated. The resulting mixture was dissolved in 4 mL of methanol with 2 drops of triethylamine and then purified by preparative HPLC, eluting with a gradient of 21-51% of acetonitrile in water containing ammonium bicarbonate to afford the title compound as an off-white solid (0.232 g, 65% yield): ¹H NMR (400 MHz, CDCl₃) δ 11.30 (s, 1H), 9.07 (s, 2H), 8.69 (d, J=4.8 Hz, 1H), 8.65 (d, J=5.2 Hz, 1H), 7.90 (td, J=7.6, 1.6 Hz, 1H), 7.75 (d, J=8.0 Hz, 1H), 7.43-7.34 (m, 2H), 5.46-5.33 (m, 1H), 3.06-2.92 (m, 1H), 2.30-2.01 (m, 6H), 1.83-1.71 (m, 2H), 1.44 (d, J=6.0 Hz, 6H); MS (ES+) m/z 454.3 (M+1).

### Example 234

### Synthesis of N-(2′-(4,4-difluorocyclohexyl)-[2,4′-bipyridin]-3′-yl)-5-fluoro-6-isopropoxynicotinamide

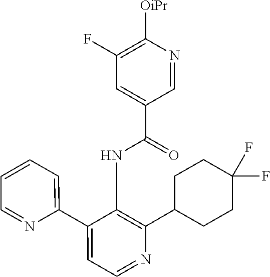 [SMILES: CC(C)Oc1ncc(C(=O)Nc2c(-c3ccccn3)ccnc2C2CCC(F)(F)CC2)cc1F]

### Step 1. Preparation of N-(2′-(4,4-difluorocyclohexyl)-[2,4′-bipyridin]-3′-yl)-5,6-difluoronicotinamide

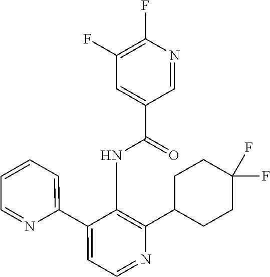 [SMILES: O=C(Nc1c(-c2ccccn2)ccnc1C1CCC(F)(F)CC1)c1cnc(F)c(F)c1]

In a similar manner as described in examples disclosed herein, utilizing the appropriately substituted starting materials and intermediates, the title compound was afforded as a yellow solid (0.0600 g, 67% yield): ¹H NMR (400 MHz, MeOD-d₄) δ 8.66-8.58 (m, 2H), 8.45 (d, J=1.6 Hz, 1H), 8.18 (dt, J=9.2, 2.0 Hz, 1H), 7.88 (dt, J=7.6, 1.6 Hz, 1H), 7.65 (d, J=8.0 Hz, 1H), 7.51 (d, J=5.2 Hz, 1H), 7.40 (dd, J=5.2, 7.2 Hz, 1H), 3.21-3.11 (m, 1H), 2.19-1.99 (m, 4H), 1.98-1.75 (m, 4H).

### Step 2. Preparation of N-(2′-(4,4-difluorocyclohexyl)-[2,4′-bipyridin]-3′-yl)-5-fluoro-6-isopropoxynicotinamide

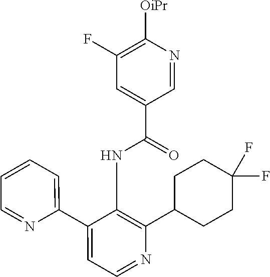 [SMILES: CC(C)Oc1ncc(C(=O)Nc2c(-c3ccccn3)ccnc2C2CCC(F)(F)CC2)cc1F]

To a solution of N-(2′-(4,4-difluorocyclohexyl)-[2,4′-bipyridin]-3′-yl)-5,6-difluoronicotinamide (0.0400 g, 0.0929 mmol) in propan-2-ol (2 mL) was added cesium carbonate (0.0908 g, 0.279 mmol). The mixture was stirred at 80° C. for 12 h. After cooling to ambient temperature, the mixture was diluted with methanol (5 mL) and filtered. The filtrate was concentrated. The residue was purified by preparative HPLC, eluting with a gradient of 48-78% of acetonitrile in water containing ammonium bicarbonate, to afford the title compound as a colorless solid (0.0214 g, 43% yield): ¹H NMR (400 MHz, MeOH-d₄) δ8.62 (d, J=5.2 Hz, 2H), 8.42 (d, J=2.0 Hz, 1H), 7.90-7.79 (m, 2H), 7.64 (d, J=8.0 Hz, 1H), 7.50 (d, J=4.8 Hz, 1H), 7.40 (ddd, J=7.6, 4.8, 0.8 Hz, 1H), 5.46 (td, J=12.4, 6.0 Hz, 1H), 3.21-3.11 (m, 1H), 2.22-1.99 (m, 4H), 1.99-1.73 (m, 4H), 1.40 (d, J=6.0 Hz, 6H); MS (ES+) m/z 292.1 (M+1).

### Example 235 and Example 236

### Synthesis of N-(2′-(4,4-difluorocyclohexyl)-[2,4′-bipyridin]-3′-yl)-2-(2-methylazetidin-1-yl)pyrimidine-5-carboxamide P1 and P2

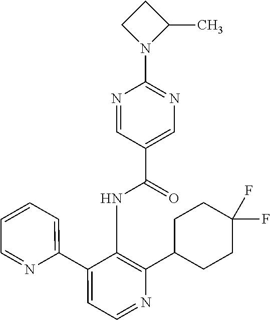 [SMILES: CC1CCN1c1ncc(C(=O)Nc2c(-c3ccccn3)ccnc2C2CCC(F)(F)CC2)cn1]

### Step 1. Preparation of N-(2′-(4,4-difluorocyclohexyl)-[2,4′-bipyridin]-3′-yl)-2-(2-methylazetidin-1-yl)pyrimidine-5-carboxamide

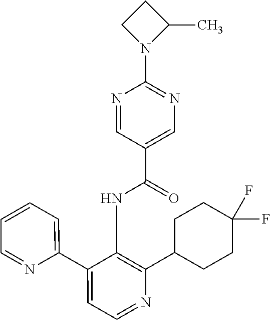 [SMILES: CC1CCN1c1ncc(C(=O)Nc2c(-c3ccccn3)ccnc2C2CCC(F)(F)CC2)cn1]

In a similar manner as described in examples disclosed herein, utilizing the appropriately substituted starting materials and intermediates, the title compound was afforded as a colorless solid (0.090 g, 67% yield): ¹H NMR (400 MHz, MeOD-d₄) S 8.69 (s, 2H), 8.65-8.58 (m, 2H), 7.87 (td, J=7.6, 1.6 Hz, 1H), 7.63 (d, J=7.6 Hz, 1H), 7.49 (d, J=5.2 Hz, 1H), 7.43-7.36 (m, 1H), 4.64-4.59 (m, 1H), 4.20-4.00 (m, 2H), 3.15 (br t, J=10.8 Hz, 1H), 2.62-2.51 (m, 1H), 2.13 (d, J=16.4 Hz, 2H), 2.08-1.98 (m, 3H), 1.97-1.74 (m, 4H), 1.54 (d, J=6.0 Hz, 3H); MS (ES+) m/z 454.1 (M+1).

### Step 2. Preparation of N-(2′-(4,4-difluorocyclohexyl)-[2,4′-bipyridin]-3′-yl)-2-(2-methylazetidin-1-yl)pyrimidine-5-carboxamide P1 and P2

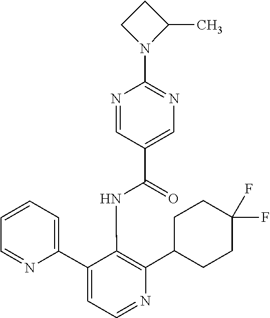 [SMILES: CC1CCN1c1ncc(C(=O)Nc2c(-c3ccccn3)ccnc2C2CCC(F)(F)CC2)cn1]

N-(2′-(4,4-difluorocyclohexyl)-[2,4′-bipyridin]-3′-yl)-2-(2-methyl-azetidin-1-yl)-pyrimidine-5-carboxamide (0.0850 g, 0.183 mmol) (dissolved in 1 mL of methanol) was purified by preparative was purified by chiral SFC (column: DAICEL CHIRALCEL OJ-H (250 mm×30 mm, 5 μm), eluting with 20% of methanol containing 0.1% ammonium hydroxide in supercritical carbon dioxide, to afford peak 1 (retention time=0.946 min as a colorless solid (0.0282 g, 33% yield, 99% ee): ¹H NMR (400 MHz, MeOD-d₄), 8.69 (s, 2H), 8.65-8.58 (m, 2H), 7.87 (td, J=8.0, 1.6 Hz, 1H), 7.63 (d, J=8.0 Hz, 1H), 7.50 (d, J=5.2 Hz, 1H), 7.40 (ddd, J=7.6, 4.8, 1.2 Hz, 1H), 4.61-4.55 (m, 1H), 4.20-4.00 (m, 2H), 3.23-3.08 (m, 1H), 2.65-2.49 (m, 1H), 2.13 (br d, J=16.4 Hz, 2H), 2.09-1.98 (m, 3H), 1.98-1.75 (m, 4H), 1.54 (d, J=6.4 Hz, 3H); MS (ES+) m/z 465.3 (M+1).

Peak 2 (retention time=1.101 min) was afforded as a colorless solid (0.0387 g, 45% yield, 99% ee): ¹H NMR (400 MHz, MeOD-d₄) δ 8.69 (s, 2H), 8.65-8.56 (m, 2H), 7.87 (td, J=7.6, 1.6 Hz, 1H), 7.63 (d, J=7.6 Hz, 1H), 7.50 (d, J=5.2 Hz, 1H), 7.43-7.36 (m, 1H), 4.60-4.53 (m, 1H), 4.22-3.99 (m, 2H), 3.21-3.08 (m, 1H), 2.63-2.51 (m, 1H), 2.22-2.09 (m, 2H), 2.09-1.98 (m, 3H), 1.98-1.87 (m, 3H), 1.86-1.75 (m, 1H), 1.54 (d, J=6.0 Hz, 3H); MS (ES+) m/z 465.3 (M+1).

### Example 237

### Synthesis of N-(4-(4-chlorophenyl)-2-(4,4-difluorocyclohexyl)pyridin-3-yl)-2-isopropylpyrimidine-5-carboxamide

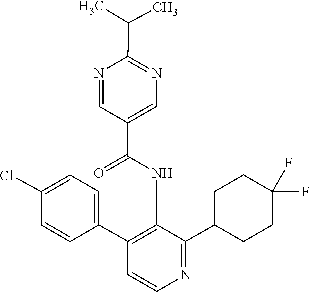 [SMILES: CC(C)c1ncc(C(=O)Nc2c(-c3ccc(Cl)cc3)ccnc2C2CCC(F)(F)CC2)cn1]

### Step 1. Preparation of tert-butyl (2-chloro-4-(4-chlorophenyl) pyridin-3-yl)carbamate

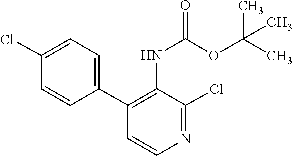 [SMILES: CC(C)(C)OC(=O)Nc1c(-c2ccc(Cl)cc2)ccnc1Cl]

To a solution of tert-butyl (2-chloro-4-iodopyridin-3-yl)carbamate (1.00 g, 2.82 mmol) and (4-chlorophenyl)boronic acid (0.463 g, 2.96 mmol) in dioxane (20 mL) and water (2 mL) was added [1,1-bis(diphenylphosphino)ferrocene]dichloropalladium(II) (0.103 g, 0.141 mmol) and potassium carbonate (1.17 g, 8.46 mmol). The mixture was stirred at 80° C. for 2.5 h under a nitrogen atmosphere. The mixture was cooled to ambient temperature, diluted with water (30 mL), and extracted with ethyl acetate (3×30 mL). The combined organic layers were washed with brine (3×30 mL), dried over sodium sulfate, filtered, and concentrated under reduced pressure. The residue was purified by flash silica gel chromatography, eluting with a gradient of 0-15% of ethyl acetate in petroleum ether, to afford the title compound as a colorless solid (0.744 g, 76% yield): ¹H NMR (400 MHz, CDCl₃) δ 8.30 (d, J=4.8 Hz, 1H), 7.45-7.37 (m, 4H), 7.21 (d, J=4.8 Hz, 1H), 6.13 (s, 1H), 1.30 (s, 9H); MS (ES+) m/z 339.0, 341.0, 343.0 (M+1).

### Step 2. Preparation of tert-butyl (4-(4-chlorophenyl)-2-(4,4-difluorocyclohexyl)pyridin-3-yl)carbamate

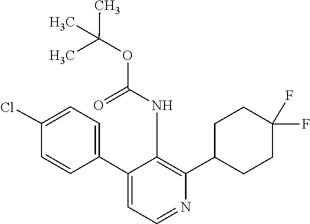 [SMILES: CC(C)(C)OC(=O)Nc1c(-c2ccc(Cl)cc2)ccnc1C1CCC(F)(F)CC1]

In a similar manner as described in examples disclosed herein, utilizing the appropriately substituted starting materials and intermediates, the title compound was afforded as a yellow solid (0.135 g, 20%): ¹H NMR (400 MHz, DMSO-d₆) δ 8.65 (s, 1H), 8.49 (d, J=4.8 Hz, 1H), 7.52 (d, J=8.4 Hz, 2H), 7.47-7.37 (m, 2H), 7.23 (d, J=4.8 Hz, 1H), 3.16-3.10 (m, 1H), 2.18-2.10 (m, 2H), 1.93-1.80 (m, 6H), 1.31 (s, 9H).

### Step 3. Preparation of 4-(4-chlorophenyl)-2-(4,4-difluorocyclohexyl) pyridin-3-amine

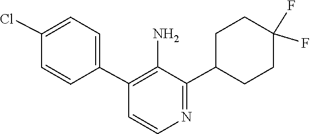 [SMILES: Nc1c(-c2ccc(Cl)cc2)ccnc1C1CCC(F)(F)CC1]

In a similar manner as described in examples disclosed herein, utilizing the appropriately substituted starting materials and intermediates, the title compound was afforded as a yellow solid (0.049 g, crude): MS (ES+) m/z 323.2, 325.2 (M+1).

### Step 4. Preparation of N-(4-(4-chlorophenyl)-2-(4,4-difluorocyclohexyl)pyridin-3-yl)-2-isopropylpyrimidine-5-carboxamide

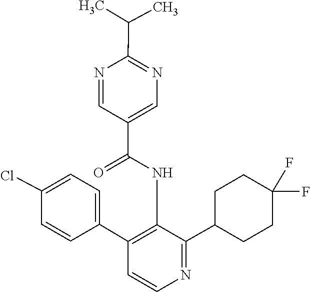 [SMILES: CC(C)c1ncc(C(=O)Nc2c(-c3ccc(Cl)cc3)ccnc2C2CCC(F)(F)CC2)cn1]

In a similar manner as described in examples disclosed herein, utilizing the appropriately substituted starting materials and intermediates, the title compound was afforded as a colorless solid (0.026 g, 51% yield): ¹H NMR (400 MHz, DMSO-d₆) δ 10.32 (s, 1H), 9.03 (s, 2H), 8.60 (d, J=4.8 Hz, 1H), 7.55-7.42 (m, 4H), 7.33 (d, J=4.8 Hz, 1H), 3.26-3.12 (m, 2H), 2.15-1.75 (m, 8H), 1.30 (d, J=6.8 Hz, 6H); MS (ES+) m/z 471.2, 473.2 (M+1).

### Example 238

### Synthesis of N-(4-(4-chlorophenyl)-2-(4,4-difluorocyclohexyl)pyridin-3-yl)-2-isopropylpyrimidine-5-carboxamide

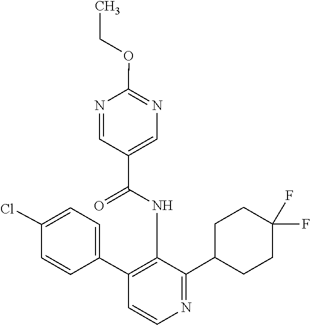 [SMILES: CCOc1ncc(C(=O)Nc2c(-c3ccc(Cl)cc3)ccnc2C2CCC(F)(F)CC2)cn1]

### Step 1. Preparation of 2-chloro-N-(4-(4-chlorophenyl)-2-(4,4-difluorocyclohexyl)pyridin-3-yl)pyrimidine-5-carboxamide

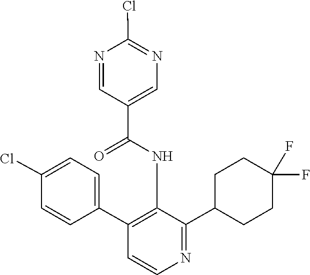 [SMILES: O=C(Nc1c(-c2ccc(Cl)cc2)ccnc1C1CCC(F)(F)CC1)c1cnc(Cl)nc1]

In a similar manner as described in examples disclosed herein, utilizing the appropriately substituted starting materials and intermediates, the title compound was afforded as a colorless solid (0.028 g, 42% yield): ¹H NMR (400 MHz, DMSO-d₆) δ 10.46 (s, 1H), 9.03 (s, 2H), 8.60 (d, J=4.8 Hz, 1H), 7.55-7.42 (m, 4H), 7.34 (d, J=4.8 Hz, 1H), 3.20-3.12 (m, 1H), 2.10-2.03 (m, 2H), 1.95-1.76 (m, 6H).

### Step 2. Preparation of N-(4-(4-chlorophenyl)-2-(4,4-difluorocyclohexyl)pyridin-3-yl)-2-isopropylpyrimidine-5-carboxamide

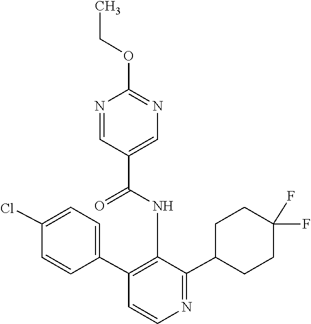 [SMILES: CCOc1ncc(C(=O)Nc2c(-c3ccc(Cl)cc3)ccnc2C2CCC(F)(F)CC2)cn1]

To a solution of sodium ethanolate (0.0206 mg, 0.302 mmol) in ethanol (2 mL) was added 2-chloro-N-(4-(4-chlorophenyl)-2-(4,4-difluorocyclohexyl)pyridin-3-yl)pyrimidine-5-carboxamide (0.0280 g, 0.0604 mmol) at 25° C. The mixture was stirred at 70° C. for 16 h. The mixture was cooled to ambient temperature and concentrated under reduced pressure. The residue was purified by preparative HPLC, eluting with a gradient of 44-74% of acetonitrile in water containing 0.225% formic acid, to afford the title compound as a colorless solid (0.0147 g, 51% yield): ¹H NMR (400 MHz, DMSO-d₆) δ 10.18 (s, 1H), 8.92 (s, 2H), 8.59 (d, J=4.8 Hz, 1H), 7.52-7.43 (m, 4H), 7.32 (d, J=4.8 Hz, 1H), 4.47-4.38 (m, 2H), 3.20-3.09 (m, 1H), 2.07 (s, 2H), 1.99-1.76 (m, 6H), 1.35 (t, J=7.2 Hz, 3H); MS (ES+) m/z 473.1, 475.1 (M+1).

### Example 239

### Synthesis of N-(2-(4,4-difluorocyclohexyl)-4-(4-fluoro-1H-pyrazol-1-yl)pyridin-3-yl)-2-isopropylpyrimidine-5-carboxamide

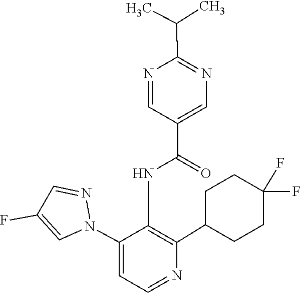 [SMILES: CC(C)c1ncc(C(=O)Nc2c(-n3cc(F)cn3)ccnc2C2CCC(F)(F)CC2)cn1]

### Step 1. Preparation of tert-butyl (2-chloro-4-(4-methyl-1H-pyrazol-1-yl)pyridin-3-yl)carbamate

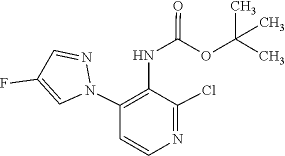 [SMILES: CC(C)(C)OC(=O)Nc1c(-n2cc(F)cn2)ccnc1Cl]

To a solution of tert-butyl (2-chloro-4-iodopyridin-3-yl)carbamate (0.500 g, 1.41 mmol) and 4-fluoro-1H-pyrazole (0.243 g, 2.82 mmol) in dimethylformamide (20 mL) were added copper iodide (0.0537 g, 0.282 mmol) and cesium carbonate (0.919 g, 2.82 mmol). The mixture was stirred at 80° C. for 16 h under a nitrogen atmosphere. The mixture was cooled to ambient temperature, diluted with water (30 mL), and extracted with ethyl acetate (3×30 mL). The combined organic layer was washed with brine (3×30 mL), dried over sodium sulfate, filtered, and concentrated under reduced pressure. The residue was purified by flash silica gel chromatography, eluting with a gradient of 0-10% of ethyl acetate in petroleum ether, followed by flash silica gel chromatography, eluting with a gradient of 0-10% of ethyl acetate in petroleum ether, to afford the title compound as a colorless solid (0.127 g, 27% yield): ¹H NMR (400 MHz, DMSO-d₆) δ 9.13 (s, 1H), 8.42 (d, J=5.2 Hz, 1H), 8.32 (d, J=4.4 Hz, 1H), 8.04-7.99 (m, 1H), 7.72 (d, J=5.2 Hz, 1H), 1.48-1.27 (m, 9H); MS (ES+) m/z 313.1, 315.1 (M+1).

### Step 2. Preparation of tert-butyl (2-(4,4-difluorocyclohexyl)-4-(4-methyl-1H-pyrazol-1-yl)pyridin-3-yl)carbamate

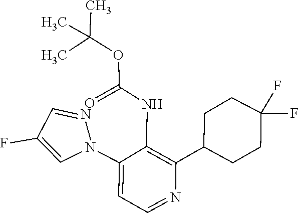 [SMILES: CC(C)(C)OC(=O)Nc1c(-n2cc(F)cn2)ccnc1C1CCC(F)(F)CC1]

In a similar manner as described in examples disclosed herein, utilizing the appropriately substituted starting materials and intermediates, the title compound was afforded as a yellow solid (0.018 g, 28% yield): ¹H NMR (400 MHz, DMSO-d₆) δ 8.84 (s, 1H), 8.55 (d, J=4.8 Hz, 1H), 8.23 (d, J=1.6 Hz, 1H), 7.93 (d, J=3.2 Hz, 1H), 7.49 (d, J=5.2 Hz, 1H), 3.19-3.09 (m, 1H), 2.18-2.11 (m, 2H), 1.93-1.77 (m, 6H), 1.38 (s, 9H); MS (ES+) m/z 297.2 (M+1).

### Step 3. Preparation of 2-(4,4-difluorocyclohexyl)-4-(4-methyl-1H-pyrazol-1-yl)pyridin-3-amine

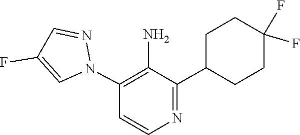 [SMILES: Nc1c(-n2cc(F)cn2)ccnc1C1CCC(F)(F)CC1]

In a similar manner as described in examples disclosed herein, utilizing the appropriately substituted starting materials and intermediates, the title compound was afforded as a yellow solid (0.030 g, crude): MS (ES+) m/z 297.2 (M+1).

### Step 4. Preparation of N-(2-(4,4-difluorocyclohexyl)-4-(4-fluoro-1H-pyrazol-1-yl)pyridin-3-yl)-2-isopropylpyrimidine-5-carboxamide

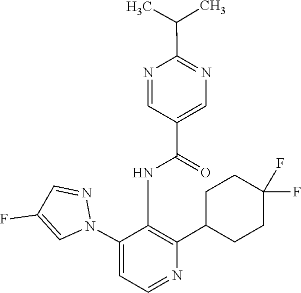 [SMILES: CC(C)c1ncc(C(=O)Nc2c(-n3cc(F)cn3)ccnc2C2CCC(F)(F)CC2)cn1]

In a similar manner as described in examples disclosed herein, utilizing the appropriately substituted starting materials and intermediates, the title compound was afforded as a colorless solid (0.011 g, 26% yield): ¹H NMR (400 MHz, DMSO-d₆) δ 10.41 (s, 1H), 9.15 (s, 2H), 8.65 (d, J=5.2 Hz, 1H), 8.39 (d, J=4.4 Hz, 1H), 7.89 (d, J=4.4 Hz, 1H), 7.54 (d, J=5.2 Hz, 1H), 3.26-3.21 (m, 2H), 2.07 (d, J=6.0 Hz, 2H), 2.01-1.77 (m, 6H), 1.32 (d, J=6.8 Hz, 6H); MS (ES+) m/z 445.1 (M+1).

### Example 240

### Synthesis of N-(4-(4,4-difluorocyclohexyl)-2-(4-fluorophenyl)pyridin-3-yl)-2-isopropylpyrimidine-5-carboxamide

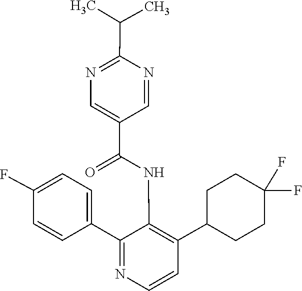 [SMILES: CC(C)c1ncc(C(=O)Nc2c(C3CCC(F)(F)CC3)ccnc2-c2ccc(F)cc2)cn1]

### Step 1. Preparation of N-(4-(4,4-difluorocyclohex-1-en-1-yl)-2-(4-fluorophenyl)pyridin-3-yl)-2-isopropylpyrimidine-5-carboxamide

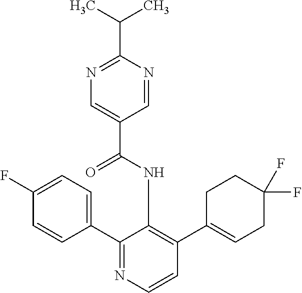 [SMILES: CC(C)c1ncc(C(=O)Nc2c(C3=CCC(F)(F)CC3)ccnc2-c2ccc(F)cc2)cn1]

To a mixture of N-(2-chloro-4-(4,4-difluorocyclohex-1-en-1-yl)pyridin-3-yl)-2-isopropylpyrimidine-5-carboxamide (0.0450 g, 0.115 mmol), (4-fluorophenyl)boronic acid (0.0481 g, 0.344 mmol) and potassium carbonate (0.0633 g, 0.458 mmol) in 1,4-dioxane (1.5 mL) and water (0.3 mL) was added [1,1′-bis(diphenylphosphino)ferrocene]dichloropalladium(II) (0.00838 g, 0.0115 mmol). The mixture was stirred at 90° C. for 12 h. The reaction mixture was cooled to ambient temperature and concentrated under reduced pressure. The residue was purified by column chromatography on silica gel, eluting with 50% of ethyl acetate in petroleum ether to afford the title compound as a colorless oil (0.0400 g, 77% yield): ¹H NMR (400 MHz, CDCl₃) δ 8.89 (s, 2H), 8.61 (d, J=4.8 Hz, 1H), 7.60-7.51 (m, 2H), 7.31 (s, 1H), 7.20 (d, J=4.8 Hz, 1H), 7.11 (t, J=8.4 Hz, 2H), 5.64 (s, 1H), 3.29 (td, J=13.6, 6.8 Hz, 1H), 2.67-2.55 (m, 4H), 2.14 (tt, J=13.6, 6.8 Hz, 2H), 1.38 (d, J=6.8 Hz, 6H).

### Step 2. Preparation of N-(4-(4,4-difluorocyclohexyl)-2-(4-fluorophenyl)pyridin-3-yl)-2-isopropylpyrimidine-5-carboxamide

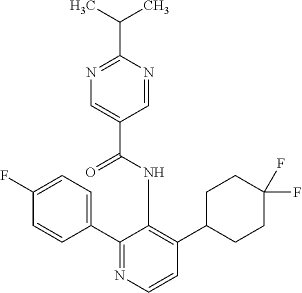 [SMILES: CC(C)c1ncc(C(=O)Nc2c(C3CCC(F)(F)CC3)ccnc2-c2ccc(F)cc2)cn1]

To a mixture of N-(4-(4,4-difluorocyclohex-1-en-1-yl)-2-(4-fluorophenyl)pyridin-3-yl)-2-isopropyl-pyrimidine-5-carboxamide (0.0300 g, 0.0663 mmol) in methanol (3 mL) was added palladium on carbon (0.0300 g, 10% purity) under a nitrogen atmosphere. The mixture was stirred at 25° C. under a hydrogen (15 psi, balloon) atmosphere for 12 h. The reaction mixture was filtered over Celite. The filtrate was concentrated under reduced pressure. The residue was purified by preparative HPLC, eluting with a gradient of 44-64% of acetonitrile in water containing 0.225% formic acid, to afford the title compound as a colorless solid (0.0191 g, 63% yield): ¹H NMR (400 MHz, DMSO-d₆) δ 10.36 (s, 1H), 9.03 (s, 2H), 8.58 (d, J=4.8 Hz, 1H), 7.62 (dd, J=8.8, 5.6 Hz, 2H), 7.47 (d, J=5.2 Hz, 1H), 7.23 (t, J=8.8 Hz, 2H), 3.21 (dt, J=13.6, 6.8 Hz, 1H), 3.07-2.93 (i, 1H), 2.11-1.83 (i, 6H), 1.76-1.65 (m, 2H), 1.30 (d, J=6.8 Hz, 6H); MS (ES+) m/z 455.2 (M+1).

| Example | Structure | Amount | MS (ES+) | |
|---|---|---|---|---|
| No. | Name | Yield | m/z | ¹H NMR |
| 241 | 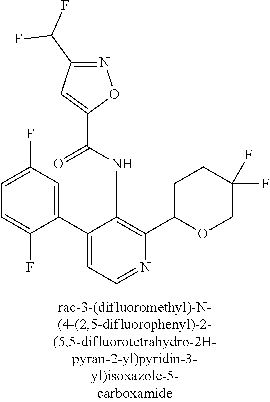 [SMILES: O=C(Nc1c(-c2cc(F)ccc2F)ccnc1C1CCC(F)(F)CO1)c1cc(C(F)F)no1] | 0.027 g 37% | 472.2 (M + 1) | (400 MHz, DMSO-d₆) δ 10.76 (s, 1H), 8.70 (d, J = 4.9 Hz, 1H), 7.54 (dd, J = 4.9, 0.8 Hz, 1H), 7.37 (t, J = 53.2 Hz, 1H), 7.47 (s, 1H), 7.39-7.24 (m, 3H), 4.95-4.92 (m, 1H), 3.93-3.86 (m, 1H), 3.83-3.72 (m, 1H), 2.33-2.15 (m, 3H), 1.99-1.94 (m, 1H) |
| 242 | 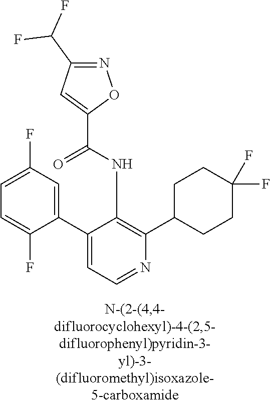 [SMILES: O=C(Nc1c(-c2cc(F)ccc2F)ccnc1C1CCC(F)(F)CC1)c1cc(C(F)F)no1] | 0.043 g 59% | 470.2 (M + 1) | (400 MHz, DMSO-d₆) δ 10.81-10.79 (m, 1H), 8.65 (d, J = 4.9 Hz, 1H), 7.45 (t, J = 19.4 Hz, 1H), 7.39 (dd, J = 4.9, 0.6 Hz, 1H), 7.36- 7.22 (m, 3H), 3.21-3.14 (m, 1H), 2.13-2.04 (m, 2H), 2.04-1.78 (m, 6H) |
| 243 | 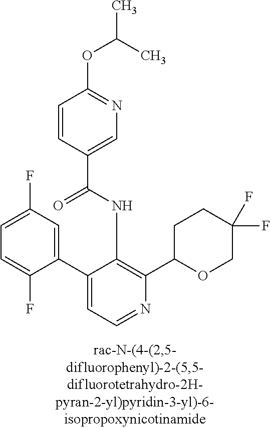 [SMILES: CC(C)Oc1ccc(C(=O)Nc2c(-c3cc(F)ccc3F)ccnc2C2CCC(F)(F)CO2)cn1] | 0.024 g 29% | 490.0 (M + 1) | (400 MHz, DMSO-d₆) δ 10.03 (s, 1H), 8.65 (d, J = 4.9 Hz, 1H), 8.57 (d, J = 2.3 Hz, 1H), 7.97 (dd, J = 8.7, 2.5 Hz, 1H), 7.51-7.50 (m, 1H), 7.34 (td, J = 9.5, 4.6 Hz, 1H), 7.26 (ddd, J = 9.6, 5.9, 3.7 Hz, 2H), 6.81 (d, J = 8.7 Hz, 1H), 5.34-5.28 (m, 1H), 4.91-4.88 (m, 1H), 3.94-3.89 (m, 1H), 3.80-3.69 (m, 1H), 2.32-2.17 (m, 3H), 1.98-1.94 (m, 1H), 1.31 (d, J = 6.2 Hz, 6H) |
| 244 | 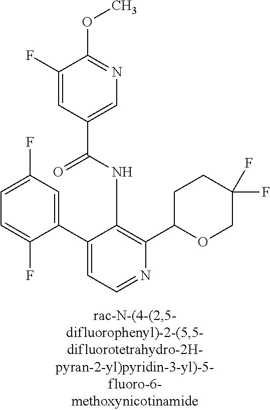 [SMILES: COc1ncc(C(=O)Nc2c(-c3cc(F)ccc3F)ccnc2C2CCC(F)(F)CO2)cc1F] | 0.046 g 61% | 480.2 (M + 1) | (400 MHz, DMSO-d₆) δ 10.14 (s, 1H), 8.67 (d, J = 4.9 Hz, 1H), 8.41 (d, J = 1.9 Hz, 1H), 7.94 (dd, J = 11.0, 2.0 Hz, 1H), 7.52 (dd, J = 4.9, 1.0 Hz, 1H), 7.34 (td, J = 9.5, 4.5 Hz, 1H), 7.30-7.25 (m, 2H), 4.91-4.88 (m, 1H), 4.03-4.01 (m, 3H), 3.93-3.89 (m, 1H), 3.81-3.70 (m, 1H), 2.32-2.23 (m, 3H), 1.98-1.94 (m, 1H) |
| 245 | 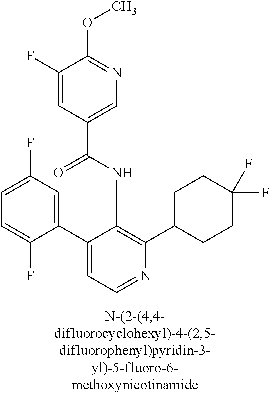 [SMILES: COc1ncc(C(=O)Nc2c(-c3cc(F)ccc3F)ccnc2C2CCC(F)(F)CC2)cc1F] | 0.048 g 33% | 478.2 (M + 1) | (400 MHz, DMSO-d₆) δ 10.14 (s, 1H), 8.62 (d, J = 4.9 Hz, 1H), 8.39 (d, J = 2.0 Hz, 1H), 7.93 (dd, J = 11.0, 2.0 Hz, 1H), 7.37 (dd, J = 4.9, 0.8 Hz, 1H), 7.33 (dt, J = 9.2, 4.6 Hz, 1H), 7.29-7.20 (m, 2H), 4.01 (s, 3H), 3.19-3.16 (m, 1H), 2.10-2.07 (m, 2H), 1.99-1.81 (m, 6H) |
| 246 | 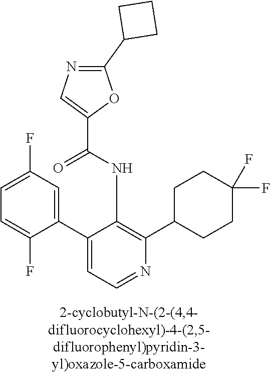 [SMILES: O=C(Nc1c(-c2cc(F)ccc2F)ccnc1C1CCC(F)(F)CC1)c1cnc(C2CCC2)o1] | 0.052 g 47% | 474.2 (M + 1) | (400 MHz, DMSO-d₆) δ 10.13 (s, 1H), 8.62 (d, J = 4.9 Hz, 1H), 7.71 (s, 1H), 7.37-7.21 (m, 4H), 3.69 (quintetd, J = 8.5, 0.9 Hz, 1H), 3.17- 3.11 (m, 1H), 2.36-2.29 (m, 4H), 2.14-1.98 (m, 3H), 1.98-1.78 (m, 7H) |
| 247 | 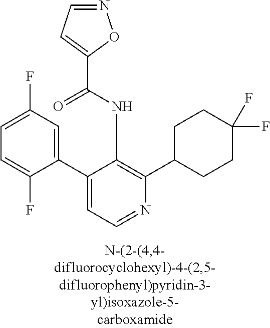 [SMILES: O=C(Nc1c(-c2cc(F)ccc2F)ccnc1C1CCC(F)(F)CC1)c1ccno1] | 0.057 g 58% | 420.0 (M + 1) | (400 MHz, DMSO-d₆) δ 10.66 (d, J = 6.5 Hz, 1H), 8.77 (t, J = 2.5 Hz, 1H), 8.64 (t, J = 3.9 Hz, 1H), 7.39 (t, J = 5.0 Hz, 1H), 7.37- 7.21 (m, 3H), 7.09 (t, J = 2.5 Hz, 1H), 3.20- 3.12 (m, 1H), 2.14-2.04 (m, 2H), 2.00-1.77 (m, 6H) |
| 248 | 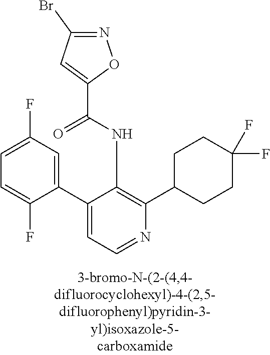 [SMILES: O=C(Nc1c(-c2cc(F)ccc2F)ccnc1C1CCC(F)(F)CC1)c1cc(Br)no1] | 0.025 g 21% | 497.8 (M + 1), 500.2 (M + 1) | (400 MHz, DMSO-d₆) δ 10.77 (s, 1H), 8.64 (d, J = 4.9 Hz, 1H), 7.43- 7.42 (m, 1H), 7.39-7.38 (m, 1H), 7.38-7.28 (m, 2H), 7.24 (ddt, J = 8.7, 5.7, 2.9 Hz, 1H), 3.19- 3.12 (m, 1H), 2.14-2.04 (m, 2H), 2.03-1.77 (m, 6H) |
| 249 | 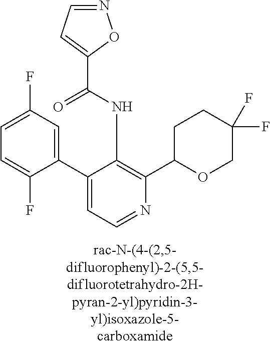 [SMILES: O=C(Nc1c(-c2cc(F)ccc2F)ccnc1C1CCC(F)(F)CO1)c1ccno1] | 0.014 g 22% | 422.0 (M + 1) | (400 MHz, DMSO-d₆) δ 10.59 (s, 1H), 8.77 (d, J = 1.9 Hz, 1H), 8.69 (d, J = 4.9 Hz, 1H), 7.53 (dd, J = 4.9, 0.9 Hz, 1H), 7.38-7.26 (m, 3H), 7.12 (d, J = 1.9 Hz, 1H), 4.93-4.90 (m, 1H), 3.92-3.87 (m, 1H), 3.80-3.69 (m, 1H), 2.33-2.18 (m, 3H), 1.99-1.95 (m, 1H) |
| 250 | 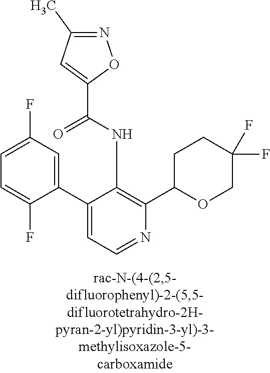 [SMILES: Cc1cc(C(=O)Nc2c(-c3cc(F)ccc3F)ccnc2C2CCC(F)(F)CO2)on1] | 0.040 g 57% | 436.2 (M + 1) | (400 MHz, DMSO-d₆) δ 10.53 (s, 1H), 8.68 (d, J = 4.9 Hz, 1H), 7.52 (dd, J = 4.9, 0.8 Hz, 1H), 7.38-7.25 (m, 3H), 6.96 (s, 1H), 4.91-4.88 (m, 1H), 3.94-3.87 (m, 1H), 3.79-3.68 (m, 1H), 2.30 (s, 3H), 2.33-2.13 (m, 3H), 1.99-1.95 (m, 1H) |
| 251 | 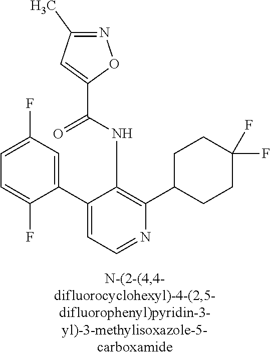 [SMILES: Cc1cc(C(=O)Nc2c(-c3cc(F)ccc3F)ccnc2C2CCC(F)(F)CC2)on1] | 0.052 g 75% | 434.2 (M + 1) | (400 MHz, DMSO-d₆) δ 10.60 (s, 1H), 8.63 (d, J = 4.9 Hz, 1H), 7.37 (dd, J = 4.8, 0.7 Hz, 1H), 7.36-7.21 (m, 3H), 6.95 (s, 1H), 3.16-3.10 (m, 1H), 2.30 (s, 3H), 2.14-2.06 (m, 2H), 1.94-1.79 (m, 6H) |
| 252 | 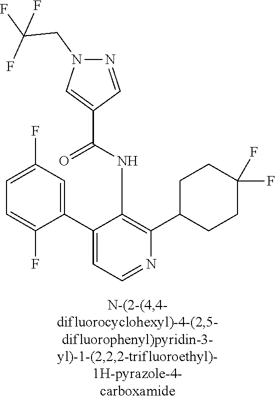 [SMILES: O=C(Nc1c(-c2cc(F)ccc2F)ccnc1C1CCC(F)(F)CC1)c1cnn(CC(F)(F)F)c1] | 0.027 g 23% | 501.2 (M + 1) | (400 MHz, DMSO-d₆) δ 9.80 (s, 1H), 8.59 (d, J = 4.9 Hz, 1H), 8.29 (s, 1H), 7.99 (s, 1H), 7.34 (d, J = 4.9 Hz, 1H), 7.33-7.17 (m, 3H), 5.24-5.17 (m, 2H), 3.18-3.12 (m, 1H), 2.14-2.06 (m, 2H), 1.97-1.78 (m, 6H) |
| 253 | 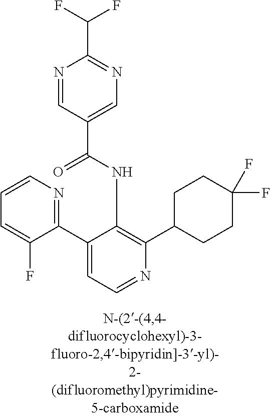 [SMILES: O=C(Nc1c(-c2ncccc2F)ccnc1C1CCC(F)(F)CC1)c1cnc(C(F)F)nc1] | 0.012 g 15% | 464.2 (M + 1) | (400 MHz, DMSO-d₆) δ 10.60 (s, 1H), 9.18 (d, J = 5.0 Hz, 2H), 8.69- 8.68 (m, 1H), 8.48 (dt, J = 4.6, 1.5 Hz, 1H), 7.85 (ddd, J = 10.2, 8.6, 1.4 Hz, 1H), 7.53- 7.49 (m, 2H), 7.20-6.94 (m, 1H), 3.27-3.20 (m, 1H), 2.14-2.07 (m, 2H), 2.02-1.86 (m, 6H) |
| 254 | 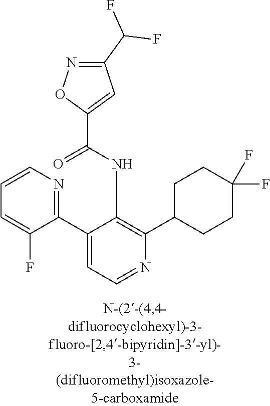 [SMILES: O=C(Nc1c(-c2ncccc2F)ccnc1C1CCC(F)(F)CC1)c1cc(C(F)F)no1] | 0.043 g 70% | 453.2 (M + 1) | (400 MHz, DMSO-d₆) δ 10.78 (s, 1H), 8.68 (d, J = 4.9 Hz, 1H), 8.47- 8.44 (m, 1H), 7.86-7.81 (m, 1H), 7.52 (dd, J = 8.7, 4.3 Hz, 1H), 7.48 (dd, J = 4.9, 1.3 Hz, 1H), 7.43-7.39 (m, 1H), 7.30 (dd, J = 52.9, 4.3 Hz, 1H), 3.22-3.15 (m, 1H), 2.16-2.05 (m, 2H), 1.99-1.84 (m, 6H) |
| 255 | 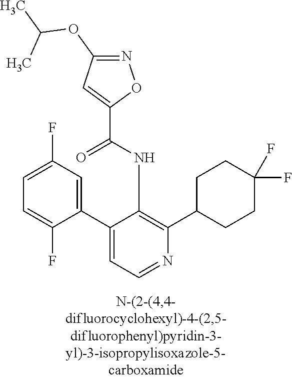 [SMILES: CC(C)Oc1cc(C(=O)Nc2c(-c3cc(F)ccc3F)ccnc2C2CCC(F)(F)CC2)on1] | 0.12 g 81% | 478.2 (M + 1) | (400 MHz, DMSO-d₆) δ 10.59 (s, 1H), 8.63 (d, J = 4.9 Hz, 1H), 7.38- 7.26 (m, 3H), 7.22 (ddd, J = 8.8, 5.6, 3.2 Hz, 1H), 6.81 (s, 1H), 4.82 (quintet, J = 6.1 Hz, 1H), 3.16-3.10 (m, 1H), 2.13-2.07 (m, 2H), 2.00-1.78 (m, 6H), 1.34 (d, J = 6.1 Hz, 6H) |
| 256 | 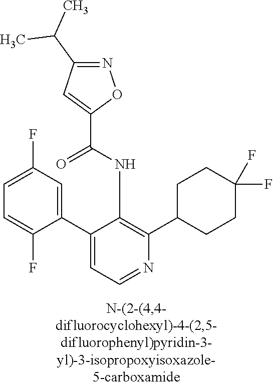 [SMILES: CC(C)c1cc(C(=O)Nc2c(-c3cc(F)ccc3F)ccnc2C2CCC(F)(F)CC2)on1] | 0.12 g 78% | 462.2 (M + 1) | (400 MHz, DMSO-d₆) δ 10.61 (s, 1H), 8.63 (d, J = 4.9 Hz, 1H), 7.37 (d, J = 4.8 Hz, 1H), 7.36-7.27 (m, 2H), 7.27-7.21 (m, 1H), 7.07 (s, 1H), 3.17-3.11 (m, 1H), 3.06 (dt, J = 13.9, 6.9 Hz, 1H), 2.14-2.05 (m, 2H), 1.98-1.79 (m, 6H), 1.23 (t, J = 6.6 Hz, 6H) |
| 257 | 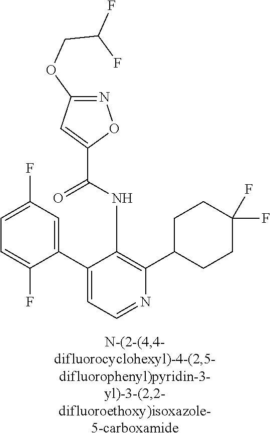 [SMILES: O=C(Nc1c(-c2cc(F)ccc2F)ccnc1C1CCC(F)(F)CC1)c1cc(OCC(F)F)no1] | 0.11 g 72% | 500.2 (M + 1) | (400 MHz, DMSO-d₆) δ 10.67 (s, 1H), 8.63 (t, J = 4.0 Hz, 1H), 7.38 (d, J = 4.9 Hz, 1H), 7.37- 7.26 (m, 2H), 7.23 (ddd, J = 8.8, 5.6, 3.1 Hz, 1H), 6.99 (d, J = 4.4 Hz, 1H), 6.42 (tt, J = 53.9, 3.1 Hz, 1H), 4.62-4.53 (m, 2H), 3.15-3.10 (m, 1H), 2.13-2.04 (m, 2H), 1.99-1.77 (m, 6H) |
| 258 | 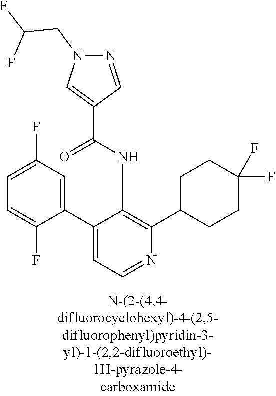 [SMILES: O=C(Nc1c(-c2cc(F)ccc2F)ccnc1C1CCC(F)(F)CC1)c1cnn(CC(F)F)c1] | 0.069 g 91% | 483.2 (M + 1) | (400 MHz, DMSO-d₆) δ 9.74 (d, J = 6.5 Hz, 1H), 8.61-8.58 (m, 1H), 8.23 (s, 1H), 7.96-7.93 (m, 1H), 7.34-7.33 (m, 1H), 7.24 (dddd, J = 29.7, 14.1, 7.4, 3.9 Hz, 3H), 6.39 (tt, J = 54.7, 3.6 Hz, 1H), 4.71-4.62 (m, 2H), 3.17-3.11 (m, 1H), 2.17-2.04 (m, 2H), 1.91-1.75 (m, 6H) |
| 259 | 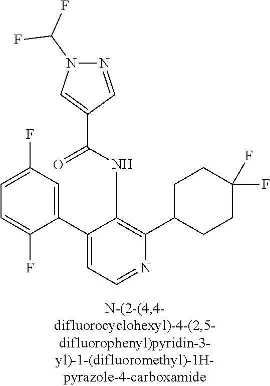 [SMILES: O=C(Nc1c(-c2cc(F)ccc2F)ccnc1C1CCC(F)(F)CC1)c1cnn(C(F)F)c1] | 0.040 g 54% | 469.2 (M + 1) | (400 MHz, DMSO-d₆) δ 9.93 (s, 1H), 8.69 (s, 1H), 8.61 (d, J = 4.9 Hz, 1H), 8.15 (s, 1H), 7.86 (t, J = 58.8 Hz, 1H), 7.36-7.35 (m, 1H), 7.34-7.19 (m, 3H), 3.16-3.15 (m, 1H), 2.11-2.07 (m, 2H), 1.94-1.80 (m, 6H) |
| 260 | 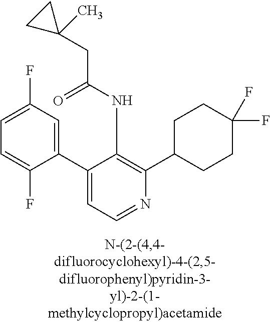 [SMILES: CC1(CC(=O)Nc2c(-c3cc(F)ccc3F)ccnc2C2CCC(F)(F)CC2)CC1] | 0.050 g 77% | 421.2 (M + 1) | (400 MHz, DMSO-d₆) δ 9.38 (s, 1H), 8.54 (d, J = 4.9 Hz, 1H), 7.36 (td, J = 9.1, 4.6 Hz, 1H), 7.33-7.27 (m, 2H), 7.16 (ddd, J = 8.8, 5.6, 3.2 Hz, 1H), 3.13-3.08 (m, 1H), 2.19-2.11 (m, 2H), 2.03 (d, J = 6.5 Hz, 2H), 1.94-1.77 (m, 6H), 0.84 (s, 3H), 0.38 (t, J = 4.4 Hz, 2H), 0.20- 0.17 (m, 2H) |
| 261 | 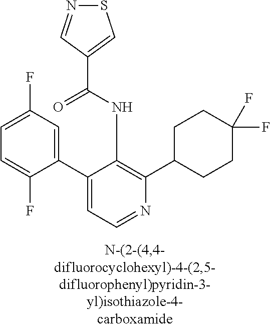 [SMILES: O=C(Nc1c(-c2cc(F)ccc2F)ccnc1C1CCC(F)(F)CC1)c1cnsc1] | 0.048 g 47% | 436.2 (M + 1) | (400 MHz, DMSO-d₆) δ 10.15 (d, J = 6.6 Hz, 1H), 9.53 (d, J = 4.1 Hz, 1H), 8.83-8.81 (m, 1H), 8.63-8.61 (m, 1H), 7.37 (d, J = 4.8 Hz, 1H), 7.36-7.27 (m, 1H), 7.27-7.21 (m, 2H), 3.23-3.15 (m, 1H), 2.11-2.04 (m, 2H), 2.02-1.80 (m, 6H) |
| 262 | 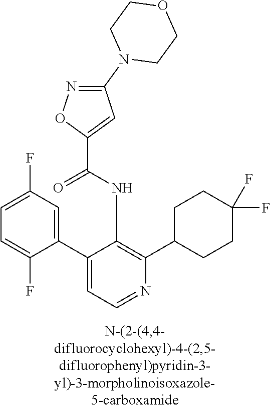 [SMILES: O=C(Nc1c(-c2cc(F)ccc2F)ccnc1C1CCC(F)(F)CC1)c1cc(N2CCOCC2)no1] | 0.12 g 78% | 505.2 (M + 1) | (400 MHz, DMSO-d₆) δ 10.57 (s, 1H), 8.62 (d, J = 4.9 Hz, 1H), 7.38- 7.26 (m, 3H), 7.24-7.20 (m, 1H), 6.97-6.96 (m, 1H), 3.72-3.65 (m, 4H), 3.25-3.18 (m, 4H), 3.16-3.08 (m, 1H), 2.15-2.04 (m, 2H), 1.99-1.77 (m, 6H) |
| 263 | 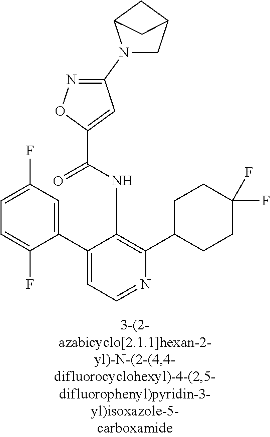 [SMILES: O=C(Nc1c(-c2cc(F)ccc2F)ccnc1C1CCC(F)(F)CC1)c1cc(N2CC3CC2C3)no1] | 0.010 g 13% | 501.2 (M + 1) | (400 MHz, DMSO-d₆) δ 10.52 (s, 1H), 8.62 (d, J = 4.9 Hz, 1H), 7.38- 7.20 (m, 4H), 6.86 (s, 1H), 4.26 (d, J = 6.7 Hz, 1H), 3.26 (s, 2H), 3.14-3.10 (m, 1H), 2.89 (dt, J = 6.4, 3.1 Hz, 1H), 2.14-2.06 (m, 2H), 1.95-1.79 (m, 8H), 1.29-1.26 (m, 2H) |
| 264 | 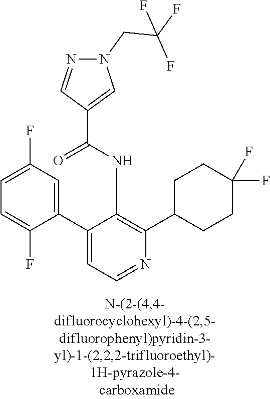 [SMILES: O=C(Nc1c(-c2cc(F)ccc2F)ccnc1C1CCC(F)(F)CC1)c1cnn(CC(F)(F)F)c1] | 0.063 g 54% | 501.2 (M + 1) | (400 MHz, DMSO-d₆) δ 9.88 (s, 1H), 8.58 (d, J = 4.9 Hz, 1H), 7.94 (d, J = 2.4 Hz, 1H), 7.34 (dd, J = 4.9, 1.2 Hz, 1H), 7.33-7.20 (m, 3H), 6.73 (d, J = 2.4 Hz, 1H), 5.25-5.18 (m, 2H), 3.17-3.09 (m, 1H), 2.14-2.06 (m, 2H), 1.95-1.81 (m, 5H), 1.81-1.71 (m, 1H) |
| 265 | 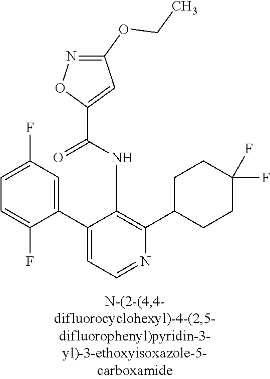 | 0.041 g 57% | 434.4 (M + 1) | (400 MHz, DMSO-d₆) δ 10.62-10.59 (m, 1H), 8.63 (d, J = 4.9 Hz, 1H), 7.38 (t, J = 4.5 Hz, 1H), 7.37-7.26 (m, 2H), 7.23 (ddd, J = 8.8, 5.6, 3.2 Hz, 1H), 6.83 (d, J = 4.1 Hz, 1H), 4.30-4.25 (m, 2H), 3.17-3.10 (m, 1H), 2.14-2.04 (m, 2H), 2.01-1.77 (m, 6H), 1.34 (q, J = 6.3 Hz, 3H) |
| 266 | 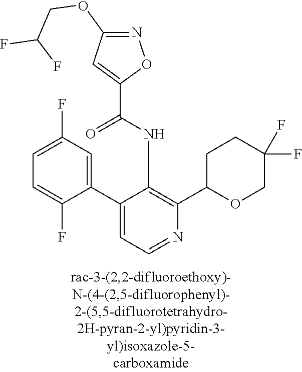 [SMILES: O=C(Nc1c(-c2cc(F)ccc2F)ccnc1C1CCC(F)(F)CO1)c1cc(OCC(F)F)no1] | 0.28 g 90% | 502.2 (M + 1) | (400 MHz, DMSO-d₆) δ 10.59 (d, J = 5.4 Hz, 1H), 8.69 (d, J = 4.9 Hz, 1H), 7.53 (d, J = 4.9 Hz, 1H), 7.39-7.25 (m, 3H), 7.00-6.99 (m, 1H), 6.42 (tt, J = 53.9, 3.1 Hz, 1H), 4.91-4.88 (m, 1H), 4.62-4.53 (m, 2H), 3.94-3.88 (m, 1H), 3.81-3.70 (m, 1H), 2.33-2.14 (m, 3H), 1.98-1.93 (m, 1H) |
| 267 | 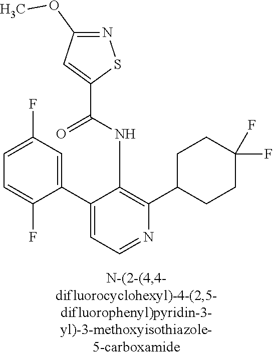 [SMILES: COc1cc(C(=O)Nc2c(-c3cc(F)ccc3F)ccnc2C2CCC(F)(F)CC2)sn1] | 0.081 g 75% | 466.2 (M + 1) | (400 MHz, DMSO-d₆) δ 10.43 (s, 1H), 8.64 (d, J = 4.9 Hz, 1H), 7.38 (t, J = 4.8 Hz, 1H), 7.36-7.25 (m, 3H), 7.22 (ddd, J = 8.8, 5.6, 3.2 Hz, 1H), 3.95 (s, 3H), 3.19-3.13 (m, 1H), 2.15-2.06 (m, 2H), 2.00-1.77 (m, 6H) |
| 268 | 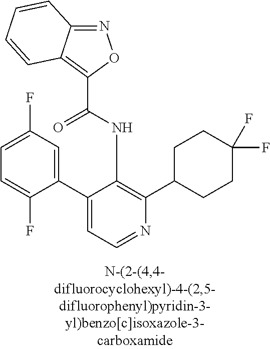 [SMILES: O=C(Nc1c(-c2cc(F)ccc2F)ccnc1C1CCC(F)(F)CC1)c1onc2ccccc12] | 0.046 g 48% | 470.2 (M + 1) | (400 MHz, DMSO-d₆) δ 10.99-10.96 (m, 1H), 8.68-8.64 (m, 1H), 7.82-7.77 (m, 2H), 7.54-7.49 (m, 1H), 7.42-7.38 (m, 1H), 7.34-7.19 (m, 4H), 3.29-3.20 (m, 1H), 2.12-2.06 (m, 2H), 1.98-1.84 (m, 6H) |
| 269 | 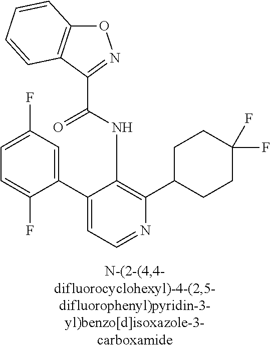 [SMILES: O=C(Nc1c(-c2cc(F)ccc2F)ccnc1C1CCC(F)(F)CC1)c1noc2ccccc12] | 0.029 g 33% | 470.2 (M + 1) | (400 MHz, DMSO-d₆) δ 10.90-10.85 (m, 1H), 8.66-8.64 (m, 1H), 7.92-7.85 (m, 2H), 7.78-7.72 (m, 1H), 7.53-7.46 (m, 1H), 7.43-7.38 (m, 1H), 7.36-7.21 (m, 3H), 3.30-3.20 (m, 1H), 2.18-2.05 (m, 2H), 1.99-1.80 (m, 6H) |
| 270 | 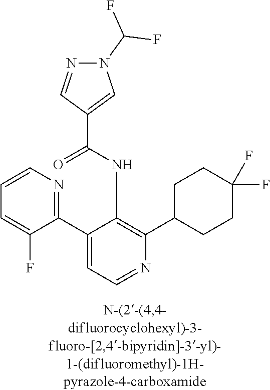 [SMILES: O=C(Nc1c(-c2ncccc2F)ccnc1C1CCC(F)(F)CC1)c1cnn(C(F)F)c1] | 0.022 g 47% | 452.2 (M + 1) | (400 MHz, DMSO-d₆) δ 9.43-9.42 (m, 1H), 8.66-8.64 (m, 1H), 8.60-8.59 (m, 1H), 8.51-8.50 (m, 1H), 8.10 (d, J = 4.3 Hz, 1H), 7.74-7.69 (m, 1H), 7.68 (t, J = 60.0, 1H), 7.52- 7.46 (m, 2H), 3.36-3.28 (m, 1H), 2.18-2.09 (m, 3H), 2.06-2.03 (m, 1H), 2.02-1.92 (m, 3H), 1.91-1.80 (m, 1H) |
| 271 | 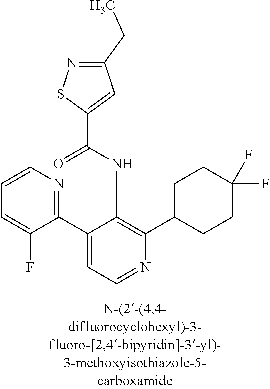 [SMILES: CCc1cc(C(=O)Nc2c(-c3ncccc3F)ccnc2C2CCC(F)(F)CC2)sn1] | 0.026 g 56% | 449.2 (M + 1) | (400 MHz, DMSO-d₆) δ 9.75-9.74 (s, 1H), 8.69 (d, J = 4.9 Hz, 1H), 8.51 (dt, J = 4.5, 1.4 Hz, 1H), 7.74 (ddd, J = 10.1, 8.7, 1.3 Hz, 1H), 7.54-7.50 (m, 2H), 7.20 (d, J = 5.3 Hz, 1H), 4.00-3.98 (m, 3H), 3.37-3.30 (m, 1H), 2.20-2.10 (m, 3H), 2.06-2.03 (m, 1H), 2.01-1.93 (m, 3H), 1.92-1.84 (m, 1H) |
| 272 | 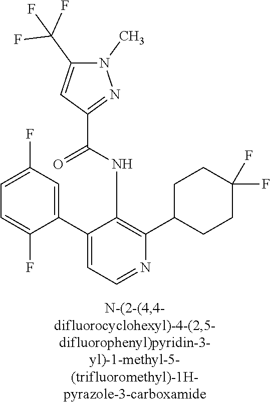 [SMILES: Cn1nc(C(=O)Nc2c(-c3cc(F)ccc3F)ccnc2C2CCC(F)(F)CC2)cc1C(F)(F)F] | 0.026 g 54% | 501.2 (M + 1) | (400 MHz, DMSO-d₆) δ 9.32-9.31 (s, 1H), 8.64 (dd, J = 5.1, 2.6 Hz, 1H), 7.35 (d, J = 4.9 Hz, 1H), 7.28-7.16 (m, 3H), 7.13-7.12 (m, 1H), 4.08-4.06 (m, 3H), 3.33-3.25 (m, 1H), 2.19-2.09 (m, 3H), 2.04-2.01 (m, 1H), 1.97-1.76 (m, 4H) |
| 273 | 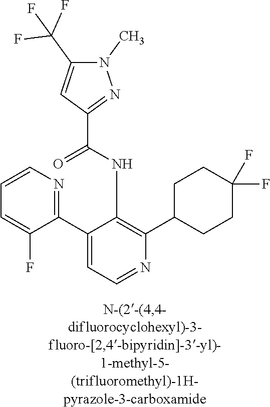 [SMILES: Cn1nc(C(=O)Nc2c(-c3ncccc3F)ccnc2C2CCC(F)(F)CC2)cc1C(F)(F)F] | 0.029 g 60% | 484.2 (M + 1) | (400 MHz, DMSO-d₆) δ 10.07-10.05 (s, 1H), 8.66-8.64 (m, 1H), 8.63-8.60 (m, 1H), 7.80-7.75 (m, 1H), 7.53 (dquintet, J = 8.8, 3.2 Hz, 2H), 7.13-7.11 (m, 1H), 4.14-4.12 (m, 3H), 3.29-3.21 (m, 1H), 2.20-1.99 (m, 6H), 1.96-1.78 (m, 2H) |
| 274 | 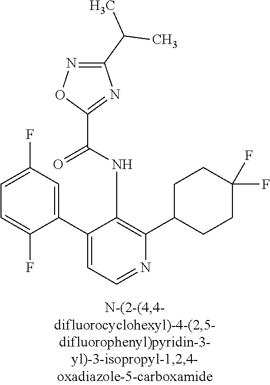 [SMILES: CC(C)c1noc(C(=O)Nc2c(-c3cc(F)ccc3F)ccnc2C2CCC(F)(F)CC2)n1] | 0.021 g 46% | 463.2 (M + 1) | (400 MHz, DMSO-d₆) δ 8.65 (d, J = 5.0 Hz, 1H), 7.38 (d, J = 5.0 Hz, 1H), 7.24-7.14 (m, 3H), 3.22-3.15 (m, 2H), 2.20-2.11 (m, 2H), 2.09-2.02 (m, 2H), 1.99-1.84 (m, 4H), 1.40-1.34 (m, 6H) |
| 275 | 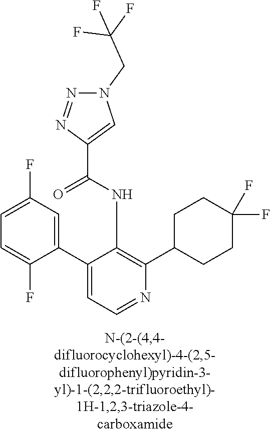 [SMILES: O=C(Nc1c(-c2cc(F)ccc2F)ccnc1C1CCC(F)(F)CC1)c1cn(CC(F)(F)F)nn1] | 0.025 g 53% | 502.2 (M + 1) | (400 MHz, DMSO-d₆) δ 10.43 (s, 1H), 8.72 (s, 1H), 8.61-8.60 (m, 1H), 7.35 (dd, J = 4.9, 1.3 Hz, 1H), 7.33-7.20 (m, 3H), 5.62-5.55 (m, 2H), 3.19-3.15 (m, 1H), 2.13-2.06 (m, 2H), 1.93-1.80 (m, 6H) |
| 276 | 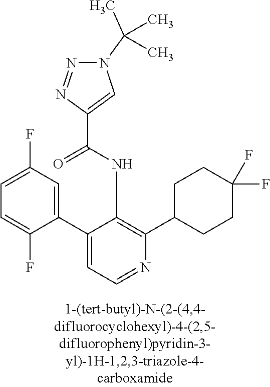 [SMILES: CC(C)(C)n1cc(C(=O)Nc2c(-c3cc(F)ccc3F)ccnc2C2CCC(F)(F)CC2)nn1] | 0.032 g 71% | 476.4 (M + 1) | (400 MHz, DMSO-d₆) δ 10.28 (s, 1H), 8.72 (s, 1H), 8.59 (d, J = 4.9 Hz, 1H), 7.34-7.21 (m, 4H), 3.19-3.12 (m, 1H), 2.14-2.05 (m, 2H), 1.97-1.74 (m, 7H), 1.63 (s, 9H) |
| 277 | 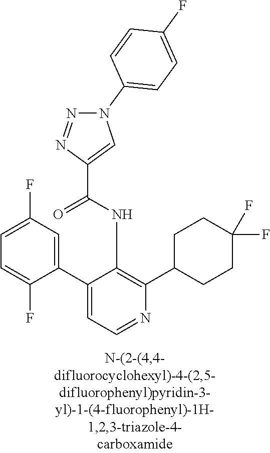 [SMILES: O=C(Nc1c(-c2cc(F)ccc2F)ccnc1C1CCC(F)(F)CC1)c1cn(-c2ccc(F)cc2)nn1] | 0.029 g 60% | 514.2 (M + 1) | (400 MHz, DMSO-d₆) δ 10.49 (s, 1H), 9.34 (s, 1H), 8.62 (d, J = 4.9 Hz, 1H), 8.02 (ddd, J = 9.6, 5.0, 2.6 Hz, 2H), 7.50-7.45 (m, 2H), 7.37-7.35 (m, 1H), 7.35-7.30 (m, 2H), 7.27-7.22 (m, 1H), 3.21-3.17 (m, 1H), 2.12-2.07 (m, 2H), 1.94-1.79 (m, 6H) |
| 278 | 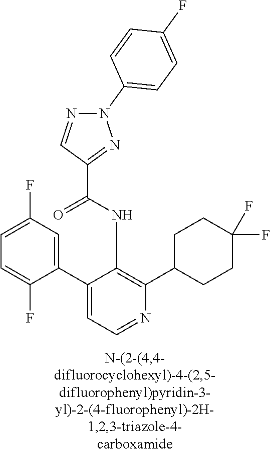 [SMILES: O=C(Nc1c(-c2cc(F)ccc2F)ccnc1C1CCC(F)(F)CC1)c1cnn(-c2ccc(F)cc2)n1] | 0.031 g 64% | 514.2 (M + 1) | (400 MHz, DMSO-d₆) δ 10.37 (s, 1H), 8.64 (d, J = 4.9 Hz, 1H), 8.47 (s, 1H), 8.13-8.07 (m, 2H), 7.53-7.47 (m, 2H), 7.39-7.38 (m, 1H), 7.36-7.22 (m, 3H), 3.23-3.18 (m, 1H), 2.13-2.06 (m, 2H), 1.98-1.83 (m, 6H) |
| 279 | 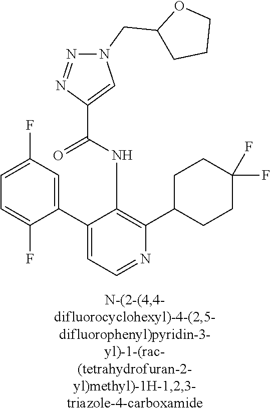 [SMILES: O=C(Nc1c(-c2cc(F)ccc2F)ccnc1C1CCC(F)(F)CC1)c1cn(CC2CCCO2)nn1] | 0.023 g 48% | 504.4 (M + 1) | (400 MHz, DMSO-d₆) δ 10.29 (s, 1H), 8.59 (d, J = 4.9 Hz, 1H), 8.50 (s, 1H), 7.34 (dd, J = 4.9, 1.3 Hz, 1H), 7.33- 7.19 (m, 3H), 4.52 (dd, J = 13.9, 3.8 Hz, 1H), 4.40 (dd, J = 14.0, 7.6 Hz, 1H), 4.23 (qd, J = 6.9, 3.7 Hz, 1H), 3.74- 3.69 (m, 1H), 3.67-3.61 (m, 1H), 3.18-3.12 (m, 1H), 2.11-2.05 (m, 2H), 2.03-1.68 (m, 9H), 1.64-1.55 (m, 1H) |
| 280 | 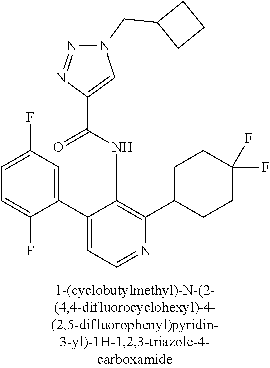 [SMILES: O=C(Nc1c(-c2cc(F)ccc2F)ccnc1C1CCC(F)(F)CC1)c1cn(CC2CCC2)nn1] | 0.034 g 71% | 488.4 (M + 1) | (400 MHz, DMSO-d₆) δ 10.27 (s, 1H), 8.59 (q, J = 4.5 Hz, 1H), 8.56 (d, J = 4.4 Hz, 1H), 7.34 (dd, J = 4.9, 1.3 Hz, 1H), 7.33-7.19 (m, 3H), 4.44-4.42 (m, 2H), 3.18-3.11 (m, 1H), 2.83-2.76 (m, 1H), 2.11-2.05 (m, 2H), 2.02-1.94 (m, 2H), 1.94-1.72 (m, 10H) |
| 281 | 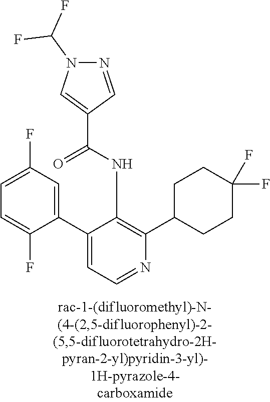 [SMILES: O=C(Nc1c(-c2cc(F)ccc2F)ccnc1C1CCC(F)(F)CC1)c1cnn(C(F)F)c1] | 0.14 g 77% | 471.2 (M + 1) | (400 MHz, CD₃CN) δ 8.62 (t, J = 3.4 Hz, 1H), 8.61-8.58 (m, 1H). 8.40-8.36 (m, 1H), 8.01-7.99 (m, 1H), 7.45-7.42 (m, 1H), 7.56-7.24 (m, 2H), 7.23-7.11 (m, 3H), 4.89-4.85 (m, 1H), 4.09-4.01 (m, 1H), 3.84-3.73 (m, 1H), 2.38-2.04 (m, 4H) |
| 282 | 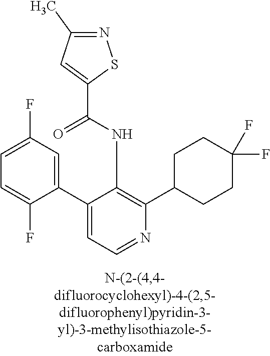 [SMILES: Cc1cc(C(=O)Nc2c(-c3cc(F)ccc3F)ccnc2C2CCC(F)(F)CC2)sn1] | 0.021 g 51% | 450.2 (M + 1) | (400 MHz, CD₃CN) δ 8.65 (d, J = 4.9 Hz, 1H), 8.63 (s, 1H), 7.45 (s, 1H), 7.33 (dd, J = 4.9, 0.7 Hz, 1H), 7.22- 7.11 (m, 3H), 3.19-3.18 (m, 1H), 2.51 (d, J = 3.3 Hz, 3H), 2.18-2.12 (m, 2H), 2.03-1.86 (m, 6H) |
| 283 | 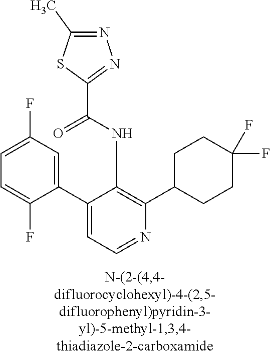 [SMILES: Cc1nnc(C(=O)Nc2c(-c3cc(F)ccc3F)ccnc2C2CCC(F)(F)CC2)s1] | 0.023 g 54% | 451.2 (M + 1) | (400 MHz, CD₃CN) δ 9.19 (s,1H), 8.67 (d, J = 4.9 Hz, 1H), 7.34 (dd, J = 4.9, 0.8 Hz, 1H), 7.21-7.13 (m, 3H), 3.24-3.18 (m, 1H), 2.79 (d, J = 3.9 Hz, 3H), 2.18-2.11 (m, 2H), 2.03-1.99 (m, 2H), 1.95-1.84 (m, 4H) |
| 284 | 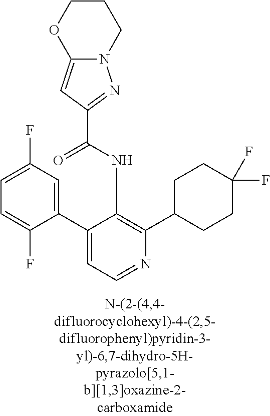 [SMILES: O=C(Nc1c(-c2cc(F)ccc2F)ccnc1C1CCC(F)(F)CC1)c1cc2n(n1)CCCO2] | 0.028 g 63% | 475.2 (M + 1) | (400 MHz, MeOD) δ 8.58 (d, J = 5.0 Hz, 1H), 7.34 (dd, J = 5.0, 0.9 Hz, 1H), 7.21-7.11 (m, 3H), 5.84 (s, 1H), 4.34 (dd, J = 5.8, 4.7 Hz, 2H), 4.21 (t, J = 6.2 Hz, 2H), 3.21-3.15 (m, 1H), 2.32-2.27 (m, 2H), 2.19-2.10 (m, 2H), 2.07-1.99 (m, 2H), 1.94-1.91 (m, 2H), 1.91-1.79 (m, 2H) |
| 285 | 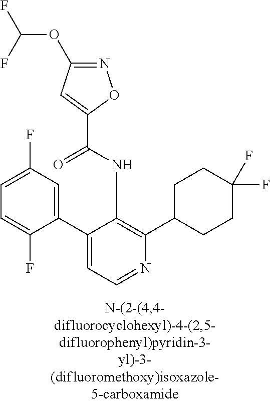 [SMILES: O=C(Nc1c(-c2cc(F)ccc2F)ccnc1C1CCC(F)(F)CC1)c1cc(OC(F)F)no1] | 0.015 g 18% | 486.2 (M + 1) | (400 MHz, MeOD) δ 8.64 (d, J = 5.0 Hz, 1H), 7.38-7.36 (m, 1H), 7.25 (t, J = 71.2 Hz, 1H), 7.23-7.11 (m, 3H), 6.87 (s, 1H), 3.18-3.12 (m, 1H), 2.21-2.11 (m, 2H), 2.09-2.02 (m, 2H), 1.97-1.81 (m, 4H) |
| 286 | 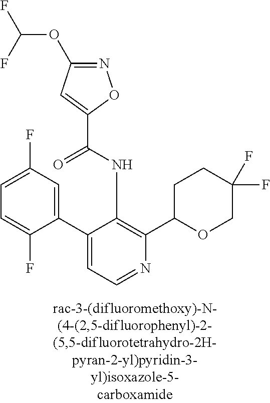 [SMILES: O=C(Nc1c(-c2cc(F)ccc2F)ccnc1C1CCC(F)(F)CO1)c1cc(OC(F)F)no1] | 0.16 g 69% | 488.2 (M + 1) | (400 MHz, MeOD) δ 8.67 (d, J = 5.0 Hz, 1H), 7.52-7.50 (m, 1H), 7.25 (t, J = 71.2 Hz, 1H), 7.25-7.16 (m, 3H), 6.88 (s, 1H), 4.93-4.90 (m, 1H), 4.05-3.98 (m, 1H), 3.82-3.71 (m, 1H), 2.37-2.05 (m, 4H) |
| 287 | 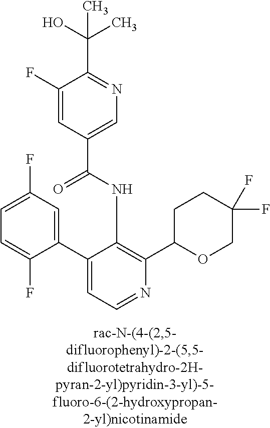 [SMILES: CC(C)(O)c1ncc(C(=O)Nc2c(-c3cc(F)ccc3F)ccnc2C2CCC(F)(F)CO2)cc1F] | 0.11 g 69% | 508.2 (M + 1) | (400 MHz, DMSO-d₆) δ 10.35 (s, 1H), 8.68 (d, J = 4.9 Hz, 1H), 8.66 (t, J = 1.6 Hz, 1H), 7.91 (dd, J = 11.9, 1.7 Hz, 1H), 7.54 (d, J = 4.9 Hz, 1H), 7.37 (td, J = 9.5, 4.6 Hz, 1H), 7.32-7.26 (m, 2H), 5.36 (s, 1H), 4.93-4.90 (m, 1H), 3.94-3.88 (m, 1H), 3.83-3.72 (m, 1H), 2.32-2.20 (m, 3H), 1.98-1.95 (m, 1H), 1.52 (s, 6H) |
| 288 | 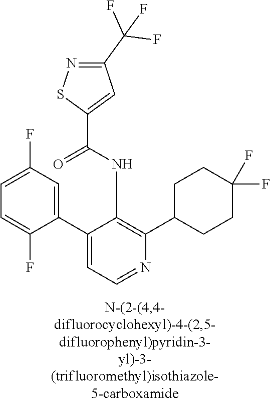 [SMILES: O=C(Nc1c(-c2cc(F)ccc2F)ccnc1C1CCC(F)(F)CC1)c1cc(C(F)(F)F)ns1] | 0.031 g 40% | 504.2 (M + 1) | (400 MHz, DMSO-d₆) δ 10.74-10.68 (m, 1H), 8.66-8.63 (m, 1H), 8.35-8.33 (m, 1H), 7.40-7.38 (m, 1H), 7.37-7.23 (m, 3H), 3.24-3.16 (m, 1H), 2.15-2.05 (m, 2H), 2.04-1.77 (m, 6H) |
| 289 | 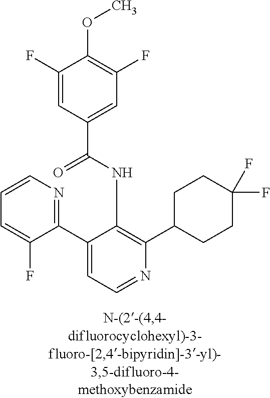 [SMILES: COc1c(F)cc(C(=O)Nc2c(-c3ncccc3F)ccnc2C2CCC(F)(F)CC2)cc1F] | 0.017 g 36% | 478.2 (M + 1) | (400 MHz, CD₃OD) δ 8.66 (d, J = 5.0 Hz, 1H), 8.47 (d, J = 4.7 Hz, 1H), 7.73-7.68 (m, 1H), 7.51-7.42 (m, 4H), 4.06 (s, 3H), 3.21-3.14 (m, 1H), 2.20-2.03 (m, 4H), 1.96-1.78 (m, 4H) |
| 290 | 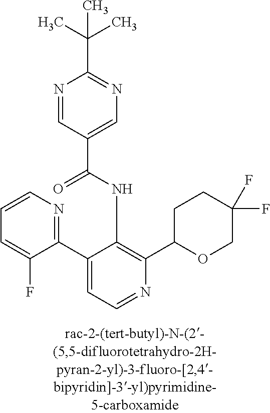 [SMILES: CC(C)(C)c1ncc(C(=O)Nc2c(-c3ncccc3F)ccnc2C2CCC(F)(F)CO2)cn1] | 0.039 g 20% | 472.2 (M + 1) | (400 MHz, DMSO-d₆) δ 10.45 (s, 1H), 8.97 (s, 2H), 8.71 (d, J = 4.9 Hz, 1H), 8.48 (dt, J = 4.6, 1.5 Hz, 1H), 7.85 (ddd, J = 10.1, 8.6, 1.3 Hz, 1H), 7.62 (dd, J = 4.9, 1.1 Hz, 1H), 7.52 (dt, J = 8.6, 4.3 Hz, 1H), 4.96-4.94 (m, 1H), 3.91-3.90 (m, 1H), 3.80 (d, J = 18.9 Hz, 1H), 2.32-2.26 (m, 3H), 2.02 (d, J = 0.3 Hz, 1H), 1.37 (s, 9H) |

### Example 291

### Synthesis of rac-N-(4-(2,5-difluorophenyl)-2-(5,5-difluorotetrahydro-2H-pyran-2-yl)pyridin-3-yl)-2-isopropoxypyrimidine-5-carboxamide

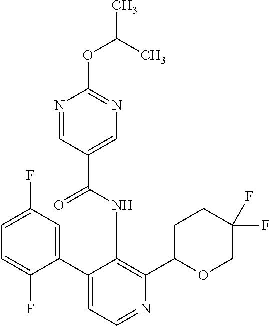 [SMILES: CC(C)Oc1ncc(C(=O)Nc2c(-c3cc(F)ccc3F)ccnc2C2CCC(F)(F)CO2)cn1]

### Step 1. Preparation of rac-2-chloro-N-(2-chloropyrimidine-5-carbonyl)-N-(4-(2,5-difluorophenyl)-2-(5,5-difluorotetrahydro-2H-pyran-2-yl)pyridin-3-yl)pyrimidine-5-carboxamide

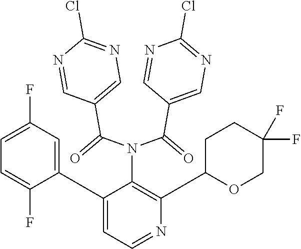 [SMILES: O=C(c1cnc(Cl)nc1)N(C(=O)c1cnc(Cl)nc1)c1c(-c2cc(F)ccc2F)ccnc1C1CCC(F)(F)CO1]

To a mixture of rac-4-(2,5-difluorophenyl)-2-(5,5-difluorotetrahydro-2H-pyran-2-yl)pyridin-3-amine (0.065 g, 0.20 mmol), 2-chloropyrimidine-5-carboxylic acid (0.51 g, 0.32 mmol) and 2-chloro-1-methylpyridinium iodide (0.14 g, 0.56 mmol) was added anhydrous tetrahydrofuran (3.3 mL). The solution was heated at 55° C. for 1 min before N-ethyl-N-isopropylpropan-2-amine (0.26 g, 2.0 mmol) was added. The reaction mixture was stirred at 55° C. for 2 h. The reaction mixture was cooled to ambient temperature and diluted with ethyl acetate (100 mL). The reaction mixture was washed with saturated ammonium chloride solution (2×50 mL). The organic layer was dried over anhydrous magnesium sulfate, filtered, and concentrated in vacuo. Purification of the residue by column chromatography, eluting with 10 to 75% of ethyl acetate in heptane, provided the title compound as a yellow solid (0.11 g, 87% yield): MS (ES+) m/z 607.2 (M+1), 609.2 (M+1).

### Step 2. Preparation of rac-N-(4-(2,5-difluorophenyl)-2-(5,5-difluorotetrahydro-2H-pyran-2-yl)pyridin-3-yl)-2-isopropoxypyrimidine-5-carboxamide

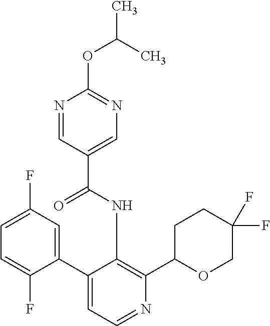 [SMILES: CC(C)Oc1ncc(C(=O)Nc2c(-c3cc(F)ccc3F)ccnc2C2CCC(F)(F)CO2)cn1]

To a mixture of rac-2-chloro-N-(2-chloropyrimidine-5-carbonyl)-N-(4-(2,5-difluorophenyl)-2-(5,5-difluorotetrahydro-2H-pyran-2-yl)pyridin-3-yl)pyrimidine-5-carboxamide (0.11 g, 0.17 mmol) in anhydrous N,N-dimethylformamide (1 mL) was added 2-propanol (0.58 mL) and 60% sodium hydride dispersion in mineral oil (0.035 g, 0.86 mmol). The solution was heated to 65° C. for 1 h. The reaction mixture was diluted with ethyl acetate (100 mL) and washed with saturated ammonium chloride solution (2×50 mL). The organic layer was dried over anhydrous magnesium sulfate, filtered, and concentrated in vacuo. Purification of the residue by column chromatography, eluting with 10 to 80% of ethyl acetate in heptane, provided the title compound as a colorless solid (0.035 g, 40% yield): ¹H-NMR (400 MHz; DMSO-d₆) S 10.21 (s, 1H), 8.89-8.86 (m, 2H), 8.67 (d, J=4.9 Hz, 1H), 7.52 (d, J=4.8 Hz, 1H), 7.38-7.32 (m, 1H), 7.28 (tq, J=7.9, 3.9 Hz, 2H), 5.27 (dt, J=12.4, 6.2 Hz, 1H), 4.92-4.90 (m, 1H), 3.95-3.88 (m, 1H), 3.84-3.73 (m, 1H), 2.32-2.15 (m, 3H), 1.98-1.93 (m, 1H), 1.35 (t, J=5.6 Hz, 6H); MS (ES+) m/z 491.0 (M+1).

### Example 292

### Synthesis of rac-2-(2,2-difluoroethoxy)-N-(4-(2,5-difluorophenyl)-2-(5,5-difluorotetrahydro-2H-pyran-2-yl)pyridin-3-yl)pyrimidine-5-carboxamide

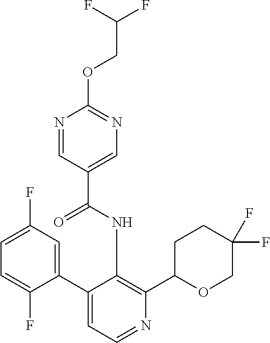 [SMILES: O=C(Nc1c(-c2cc(F)ccc2F)ccnc1C1CCC(F)(F)CO1)c1cnc(OCC(F)F)nc1]

To a mixture of rac-2-chloro-N-(2-chloropyrimidine-5-carbonyl)-N-(4-(2,5-difluorophenyl)-2-(5,5-difluorotetrahydro-2H-pyran-2-yl)pyridin-3-yl)pyrimidine-5-carboxamide (0.15 g, 0.25 mmol) in anhydrous N,N-dimethylformamide (1.2 mL) was added 2.2-difluoroethanol (2.5 mL) and 60% sodium hydride dispersion in mineral oil (0.049 g, 1.2 mmol). The solution was heated to 45° C. for 1 h. The reaction mixture was diluted with ethyl acetate (100 mL) and washed with 1M sodium hydroxide (50 mL), and saturated ammonium chloride solution (2×50 mL). The organic layer was dried over anhydrous magnesium sulfate, filtered, and concentrated in vacuo. Purification of the residue by column chromatography, eluting with 5 to 60% of ethyl acetate in heptane, provided the title compound as a colorless solid (0.090 g, 70% yield): ¹H-NMR (400 MHz; DMSO-d₆) δ 10.32 (s, 1H), 8.94 (s, 2H), 8.68 (d, J=4.9 Hz, 1H), 7.53 (dd, J=4.9, 0.7 Hz, 1H), 7.39-7.33 (m, 1H), 7.32-7.26 (m, 2H), 6.44 (tt, J=54.3, 3.4 Hz, 1H), 4.92 (d, J=10.5 Hz, 1H), 4.71 (td, J=15.0, 3.4 Hz, 2H), 3.93-3.89 (m, 1H), 3.84-3.73 (m, 1H), 2.32-2.24 (m, 3H), 1.98-1.95 (m, 1H); MS (ES+) m/z 513.2 (M+1).

### Example 293

### Synthesis of N-(2-(4,4-difluorocyclohexyl)-4-(2,5-difluorophenyl)pyridin-3-yl)-6-isopropoxynicotinamide

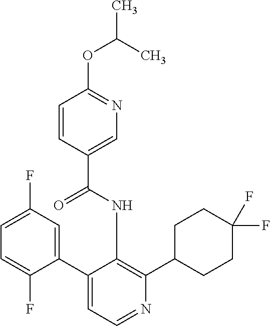 [SMILES: CC(C)Oc1ccc(C(=O)Nc2c(-c3cc(F)ccc3F)ccnc2C2CCC(F)(F)CC2)cn1]

### Step 1. Preparation of a mixture of N-(2-(4,4-difluorocyclohexyl)-4-(2,5-difluorophenyl)pyridin-3-yl)-6-fluoronicotinamide and N-(2-(4,4-difluorocyclohexyl)-4-(2,5-difluorophenyl)pyridin-3-yl)-6-fluoro-N-(6-fluoronicotinoyl)nicotinamide

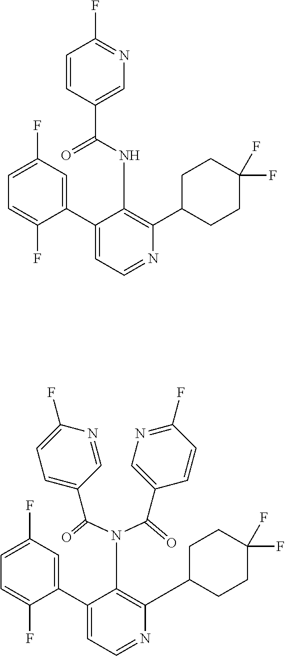 [SMILES: O=C(Nc1c(-c2cc(F)ccc2F)ccnc1C1CCC(F)(F)CC1)c1ccc(F)nc1 ; O=C(c1ccc(F)nc1)N(C(=O)c1ccc(F)nc1)c1c(-c2cc(F)ccc2F)ccnc1C1CCC(F)(F)CC1]

To a mixture of 2-(4,4-difluorocyclohexyl)-4-(2,5-difluorophenyl)pyridin-3-amine (0.10 g, 0.31 mmol), 6-fluoronicotinic acid (0.052 g, 0.37 mmol) and 2-chloro-1-methylpyridinium iodide (0.19 g, 0.74 mmol) was added anhydrous tetrahydrofuran (5.1 mL). The solution was heated at 55° C. for 1 min before N-ethyl-N-isopropylpropan-2-amine (0.40 g, 3.1 mmol) was added. The reaction mixture was stirred at 60° C. for 2 h. The reaction mixture was cooled to ambient temperature and diluted with ethyl acetate (100 mL). The reaction mixture was washed with 1M sodium hydroxide (20 mL), saturated ammonium chloride solution (2×50 mL). The organic layer was dried over anhydrous magnesium sulfate, filtered, and concentrated in vacuo. Purification of the residue by column chromatography, eluting with 5 to 80% of ethyl acetate in heptane, provided the title compounds as a colorless oil (0.14 g, 100% yield): MS (ES+) m/z 448.2 (M+1), 571.2 (M+1).

### Step 2. Preparation of a mixture of N-(2-(4,4-difluorocyclohexyl)-4-(2,5-difluorophenyl)pyridin-3-yl)-6-isopropoxynicotinamide

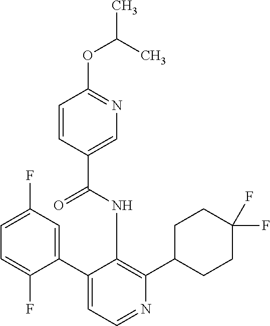 [SMILES: CC(C)Oc1ccc(C(=O)Nc2c(-c3cc(F)ccc3F)ccnc2C2CCC(F)(F)CC2)cn1]

To a mixture of N-(2-(4,4-difluorocyclohexyl)-4-(2,5-difluorophenyl)pyridin-3-yl)-6-fluoronicotinamide and N-(2-(4,4-difluorocyclohexyl)-4-(2,5-difluorophenyl)pyridin-3-yl)-6-fluoro-N-(6-fluoronicotinoyl)nicotinamide (0.14 g, 0.32 mmol) was added potassium tert-butoxide (0.36 g, 3.2 mmol), 2-propanol (0.25 mL), and anhydrous 1,4-dioxane (1.6 mL). The reaction vessel was sealed and heated to 90° C. for 1 h. The reaction mixture was cooled to ambient temperature, diluted with ethyl acetate (100 mL) and washed with 1M sodium hydroxide solution (30 mL), and saturated ammonium chloride solution (2×50 mL). The organic layer was dried over anhydrous magnesium sulfate, filtered and concentrated in vacuo. Purification of the residue by column chromatography, eluting with 10 to 80% of ethyl acetate in heptane, provided the title compound as a colorless solid (0.054 g, 34% yield): ¹H-NMR (400 MHz; DMSO-d₆) δ 10.03 (s, 1H), 8.61 (d, J=4.9 Hz, 1H), 8.55 (d, J=2.2 Hz, 1H), 7.96 (dd, J=8.7, 2.5 Hz, 1H), 7.36 (d, J=4.4 Hz, 1H), 7.33 (td, J=8.8, 4.2 Hz, 1H), 7.28-7.20 (m, 2H), 6.81 (d, J=8.7 Hz, 1H), 5.30 (dt, J=12.4, 6.2 Hz, 1H), 3.20-3.15 (m, 1H), 2.11-2.06 (m, 2H), 1.98-1.80 (m, 6H), 1.31 (d, J=6.2 Hz, 6H); MS (ES+) m/z 488.2 (M+1).

### Example 294

### Synthesis of rac-N-(2-(4,4-difluorocyclohexyl)-4-(2,5-difluorophenyl)pyridin-3-yl)-6-(tetrahydro-2H-pyran-2-yl)nicotinamide

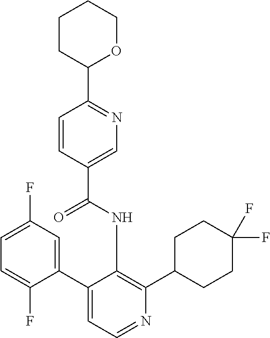 [SMILES: O=C(Nc1c(-c2cc(F)ccc2F)ccnc1C1CCC(F)(F)CC1)c1ccc(C2CCCCO2)nc1]

### Step 1. Preparation of 6-chloro-N-(2-(4,4-difluorocyclohexyl)-4-(2,5-difluorophenyl)pyridin-3-yl)nicotinamide

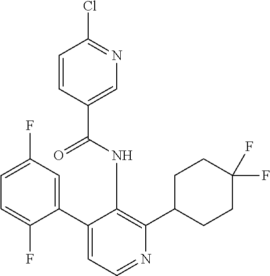 [SMILES: O=C(Nc1c(-c2cc(F)ccc2F)ccnc1C1CCC(F)(F)CC1)c1ccc(Cl)nc1]

To a mixture of 2-(4,4-difluorocyclohexyl)-4-(2,5-difluorophenyl)pyridin-3-amine (0.52 g, 1.6 mmol), 6-chloronicotinic acid (0.38 g, 2.4 mmol) and 2-chloro-1-methylpyridinium iodide (1.3 g, 5.1 mmol) was added anhydrous tetrahydrofuran (32 mL). The solution was heated at 60° C. for 1 min before N-ethyl-N-isopropylpropan-2-amine (2.1 g, 16 mmol) was added. The reaction mixture was stirred at 60° C. for 2 h. The reaction mixture was cooled to ambient temperature and diluted with methanol (5 mL), and 10M sodium hydroxide solution (3 mL) and stirred for 1 h. The reaction mixture was further diluted with ethyl acetate (100 mL) and washed with 1M sodium hydroxide (50 mL), and saturated ammonium chloride solution (2×50 mL). The organic layer was dried over anhydrous magnesium sulfate, filtered, and concentrated in vacuo. Purification of the residue by column chromatography, eluting with 10 to 50% of ethyl acetate in heptane, provided the title compounds as a colorless solid (0.41 g, 56% yield): MS (ES+) m/z 464.2 (M+1); 466.2 (M+1).

### Step 2. Preparation of rac-N-(2-(4,4-difluorocyclohexyl)-4-(2,5-difluorophenyl)pyridin-3-yl)-6-(tetrahydro-2H-pyran-2-yl)nicotinamide

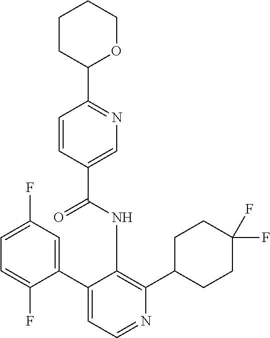 [SMILES: O=C(Nc1c(-c2cc(F)ccc2F)ccnc1C1CCC(F)(F)CC1)c1ccc(C2CCCCO2)nc1]

To 6-chloro-N-(2-(4,4-difluorocyclohexyl)-4-(2,5-difluorophenyl)pyridin-3-yl)nicotinamide (0.050 g, 0.11 mmol) was added tetrahydropyran-2-carboxylic acid (0.24 g, 0.18 mmol), (4,4″-di-t-butyl-2,2″-bipyridine)bis[3,5-difluoro-2-[5-trifluoromethyl-2-pyridinyl-kN)phenyl-kC]iridium(II) hexafluorophosphate (0.0012 g, 0.0011 mmol), dichloro(dimethoxyethane)nickel (0.0024 g, 0.011 mmol), 4-tert-butyl-2-(4-tert-butyl-2-pyridyl)pyridine (0.0043 g, 0.016 mmol), cesium carbonate (0.063 g, 0.19 mmol), and N,N-dimethylformamide (1.8 mL). The headspace of the vial was replaced with nitrogen, the vial was sealed, and the reaction mixture was stirred in front of Kessil PR160L lights (440 nm) for 24 h. The reaction mixture was diluted with ethyl acetate (50 mL) and washed with 1M sodium hydroxide (25 mL), saturated ammonium chloride solution (2×25 mL), dried over anhydrous magnesium sulfate, filtered, and concentrated in vacuo. Purification of the residue by column chromatography, eluting with 5 to 60% of ethyl acetate in heptane, afforded the title compound as a colorless solid (0.27 g, 45% yield): ¹H-NMR (400 MHz; DMSO-d₆) δ 10.25 (s, 1H), 8.80 (dd, J=2.3, 0.8 Hz, 1H), 8.62 (t, J=4.2 Hz, 1H), 8.06 (dd, J=8.2, 2.3 Hz, 1H), 7.53 (d, J=8.2 Hz, 1H), 7.38-7.36 (m, 1H), 7.35-7.31 (m, 1H), 7.30-7.21 (m, 2H), 4.42 (dd, J=11.2, 2.2 Hz, 1H), 4.09-4.04 (m, 1H), 3.61-3.55 (m, 1H), 3.23-3.15 (m, 1H), 2.15-2.04 (m, 2H), 2.03-1.82 (m, 8H), 1.73-1.62 (m, 1H), 1.61-1.52 (m, 2H), 1.43-1.33 (m, 1H); MS (ES+) m/z 514.2 (M+1).

### Example 295

### Synthesis of rac-N-(2-(4,4-difluorocyclohexyl)-4-(2,5-difluorophenyl)pyridin-3-yl)-6-(tetrahydrofuran-2-yl)nicotinamide

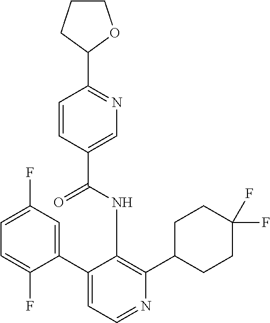 [SMILES: O=C(Nc1c(-c2cc(F)ccc2F)ccnc1C1CCC(F)(F)CC1)c1ccc(C2CCCO2)nc1]

To 6-chloro-N-(2-(4,4-difluorocyclohexyl)-4-(2,5-difluorophenyl)pyridin-3-yl)nicotinamide (0.050 g, 0.11 mmol) was added tetrahydrofuran-2-carboxylic acid (0.21 g, 0.18 mmol), (4,4″-Di-t-butyl-2,2″-bipyridine)bis[3,5-difluoro-2-[5-trifluoromethyl-2-pyridinyl-kN)phenyl-kC]iridium(II) hexafluorophosphate (0.0012 g, 0.0011 mmol), dichloro(dimethoxyethane)nickel (0.0024 g, 0.011 mmol), 4-tert-butyl-2-(4-tert-butyl-2-pyridyl)pyridine (0.0043 g, 0.016 mmol), cesium carbonate (0.063 g, 0.19 mmol), and N,N-dimethylformamide (1.8 mL). The headspace of the vial was replaced with nitrogen, the vial was sealed, and the reaction mixture was stirred in front of Kessil PR160L lights (440 nm) for 24 h. The reaction mixture was diluted with ethyl acetate (50 mL) and washed with 1M sodium hydroxide (25 mL), saturated ammonium chloride solution (2×25 mL), dried over anhydrous magnesium sulfate, filtered, and concentrated in vacuo. Purification of the residue by column chromatography, eluting with 5 to 60% of ethyl acetate in heptane followed by reverse phase column chromatography, eluting with a gradient of 20 to 90% acetonitrile in water with 0.5% formic acid, afforded the title compound as a colorless solid (0.17 g, 30% yield): ¹H-NMR (400 MHz; DMSO-d₆) δ 10.27-10.23 (m, 1H), 8.83-8.80 (m, 1H), 8.64-8.61 (m, 1H), 8.07-8.04 (m, 1H), 7.54-7.52 (m, 1H), 7.37 (dd, J=4.9, 0.8 Hz, 1H), 7.37-7.31 (m, 1H), 7.29-7.22 (m, 2H), 4.96-4.93 (m, 1H), 4.03-3.97 (m, 1H), 3.90-3.85 (m, 1H), 3.23-3.15 (m, 1H), 2.39-2.30 (m, 1H), 2.16-2.05 (m, 2H), 2.02-1.79 (m, 9H); MS (ES+) m/z 500.2 (M+1).

### Example 296 and 297

### Synthesis of N-(4-(2,5-difluorophenyl)-2-(4-fluorocyclohexyl)pyridin-3-yl)-6-isopropoxynicotinamide P1 & P2

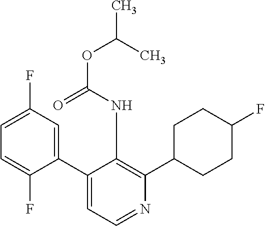 [SMILES: CC(C)OC(=O)Nc1c(-c2cc(F)ccc2F)ccnc1C1CCC(F)CC1]

### Step 1. Preparation of tert-butyl (4-(2,5-difluorophenyl)-2-(4-fluorocyclohexyl)pyridin-3-yl)carbamate

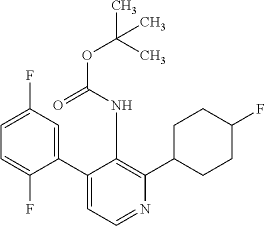 [SMILES: CC(C)(C)OC(=O)Nc1c(-c2cc(F)ccc2F)ccnc1C1CCC(F)CC1]

To tert-butyl N-[2-chloro-4-(2,5-difluorophenyl)-3-pyridyl]carbamate (0.50 g, 1.5 mmol) was added 4-fluorocyclohexanecarboxylic acid (0.28 g, 1.9 mmol), (4,4″-Di-t-butyl-2,2″-bipyridine)bis[3,5-difluoro-2-[5-trifluoromethyl-2-pyridinyl-kN)phenyl-kC]iridium(III) hexafluorophosphate (0.017 g, 0.015 mmol), dichloro(dimethoxyethane)nickel (0.032 g, 0.15 mmol), 4-tert-butyl-2-(4-tert-butyl-2-pyridyl)pyridine (0.059 g, 0.22 mmol), cesium carbonate (0.67 g, 2.1 mmol), and N,N-dimethylformamide (24 mL). The headspace of the vial was replaced with nitrogen, the vial was sealed, and the reaction mixture was stirred in front of Kessil PR160L lights (440 nm) for 24 h. The reaction mixture was diluted with ethyl acetate (200 mL) and washed with saturated ammonium chloride solution (2×50 mL), dried over anhydrous magnesium sulfate, filtered, and concentrated in vacuo. Purification of the residue by column chromatography, eluting with 0-40% of ethyl acetate in heptane, afforded the title compound as a colorless solid (0.27 g, 45% yield): MS (ES+) m/z 407.4 (M+1); 407.4 (M+1).

### Step 2. Preparation of 4-(2,5-difluorophenyl)-2-(4-fluorocyclohexyl)pyridin-3-amine

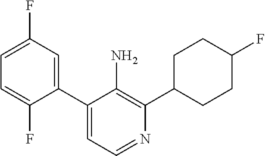 [SMILES: Nc1c(-c2cc(F)ccc2F)ccnc1C1CCC(F)CC1]

To tert-butyl (4-(2,5-difluorophenyl)-2-(4-fluorocyclohexyl)pyridin-3-yl)carbamate (0.27 g, 0.66 mmol) was added a 4 M solution of hydrochloric acid in 1,4-dioxane (3.3 mL). The reaction mixture was stirred for 18 h. The reaction mixture was diluted with ethyl acetate (200 mL), washed with 5M sodium hydroxide (10 mL), dried over anhydrous magnesium sulfate, filtered and concentrated in vacuo. The residue was purified by column chromatography eluting with a gradient of 0 to 50% of ethyl acetate in heptane to provide the title compound as a slightly yellow oil (0.19 g, 94% yield): MS (ES+) m/z 307.2 (M+1).

### Step 3. Preparation of N-(4-(2,5-difluorophenyl)-2-(4-fluorocyclohexyl)pyridin-3-yl)-6-fluoronicotinamide

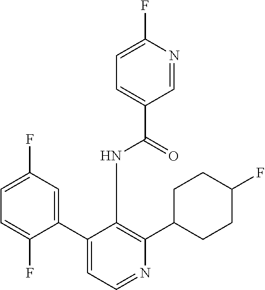 [SMILES: O=C(NCc1c(-c2cc(F)ccc2F)ccnc1C1CCC(F)CC1)c1ccc(F)nc1]

To a mixture of 4-(2,5-difluorophenyl)-2-(4-fluorocyclohexyl)pyridin-3-amine (0.19 g, 0.62 mmol), 6-fluoronicotinic acid (0.12 g, 0.82 mmol) and 2-chloro-1-methylpyridinium iodide (0.43 g, 1.7 mmol) was added anhydrous tetrahydrofuran (10 mL). The solution was heated at 65° C. for 1 min before N-ethyl-N-isopropylpropan-2-amine (0.80 g, 1.1 mmol) was added. The reaction mixture was stirred at 65° C. for 7 h. The reaction mixture was cooled to ambient temperature and diluted with ethyl acetate (125 mL). The reaction mixture was washed with saturated ammonium chloride solution (2×50 mL). The organic layer was dried over anhydrous magnesium sulfate, filtered, and concentrated in vacuo. Purification of the residue by column chromatography, eluting with 5 to 75% of ethyl acetate in heptane, provided the title compound as a colorless solid (0.23 g, 88% yield): MS (ES+) m/z 430.2 (M+1), 430.2 (M+1).

### Step 4. Preparation of N-(4-(2,5-difluorophenyl)-2-(4-fluorocyclohexyl)pyridin-3-yl)-6-isopropoxynicotinamide P1 & P2

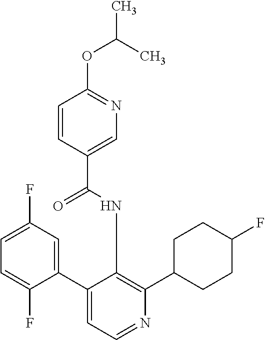 [SMILES: CC(C)Oc1ccc(C(=O)NCc2c(-c3cc(F)ccc3F)ccnc2C2CCC(F)CC2)cn1]

To a mixture of N-(4-(2,5-difluorophenyl)-2-(4-fluorocyclohexyl)pyridin-3-yl)-6-fluoronicotinamide (0.23 g, 0.54 mmol) was added potassium tert-butoxide (0.61 g, 5.4 mmol), 2-propanol (0.41 mL), and anhydrous 1,4-dioxane (2.7 mL). The reaction vessel was sealed and heated to 90° C. for 2 h. The reaction mixture was cooled to ambient temperature, diluted with ethyl acetate (150 mL) and washed with saturated ammonium chloride solution (2×50 mL). The organic layer was dried over anhydrous magnesium sulfate, filtered, and concentrated in vacuo. Purification of the residue by column chromatography, eluting with 10 to 75% of ethyl acetate in heptane followed by reverse phase column chromatography, eluting with 30 to 85% acetonitrile in water with 0.5% formic acid, provided the title compounds as colorless solids N-(4-(2,5-difluorophenyl)-2-(4-fluorocyclohexyl)pyridin-3-yl)-6-isopropoxynicotinamide

P1 (0.024 g, 18% yield): ¹H-NMR (400 MHz; DMSO-d₆) δ 10.02 (s, 1H), 8.61 (d, J=4.9 Hz, 1H), 8.54 (dd, J=2.5, 0.6 Hz, 1H), 7.96 (dd, J=8.7, 2.5 Hz, 1H), 7.35-7.30 (m, 2H), 7.27-7.20 (m, 2H), 6.81 (dd, J=8.7, 0.6 Hz, 1H), 5.30 (quintet, J=6.2 Hz, 1H), 4.93-4.80 (m, 1H), 3.12-3.06 (m, 1H), 2.04-1.90 (m, 4H), 1.67-1.51 (m, 4H), 1.31 (d, J=6.2 Hz, 6H); MS (ES+) m/z 470.2 (M+1). N-(4-(2,5-difluorophenyl)-2-(4-fluorocyclohexyl)pyridin-3-yl)-6-isopropoxynicotinamide
P2 (0.039 g, 30% yield): ¹H-NMR (400 MHz; DMSO-d₆) δ 10.03 (s, 1H), 8.58 (d, J=4.9 Hz, 1H), 8.54 (dd, J=2.5, 0.6 Hz, 1H), 7.97 (dd, J=8.7, 2.5 Hz, 1H), 7.35-7.30 (m, 2H), 7.28-7.19 (m, 2H), 6.82 (dd, J=8.7, 0.6 Hz, 1H), 5.31 (quintet, J=6.2 Hz, 1H), 4.69-4.55 (m, 1H), 3.04-2.98 (m, 1H), 2.14-2.09 (m, 2H), 1.82-1.70 (m, 4H), 1.51-1.42 (m, 2H), 1.30 (t, J=7.0 Hz, 6H); MS (ES+) m/z 470.2 (M+1).

### Example 298

### Synthesis of N-(2-(4,4-difluorocyclohexyl)-4-(2,5-difluorophenyl)pyridin-3-yl)-2-ethoxypyrimidine-5-carboxamide

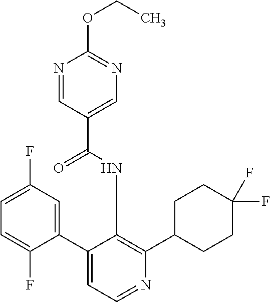 [SMILES: CCOc1ncc(C(=O)NCc2c(-c3cc(F)ccc3F)ccnc2C2CCC(F)(F)CC2)cn1]

### Step 1. Preparation of 2-chloro-N-(2-(4,4-difluorocyclohexyl)-4-(2,5-difluorophenyl)pyridin-3-yl)pyrimidine-5-carboxamide

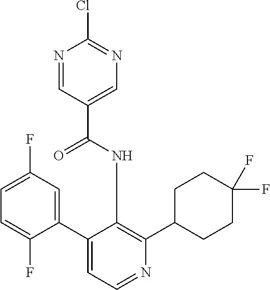 [SMILES: O=C(NCc1c(-c2cc(F)ccc2F)ccnc1C1CCC(F)(F)CC1)c1cnc(Cl)nc1]

To a mixture of 2-(4,4-difluorocyclohexyl)-4-(2,5-difluorophenyl)pyridin-3-amine (0.15 g, 0.46 mmol), 2-chloropyrimidine-5-carboxylic acid (0.088 g, 0.56 mmol) and 2-chloro-1-methylpyridinium iodide (0.30 g, 1.2 mmol) was added anhydrous tetrahydrofuran (7.7 mL). The solution was heated at 60° C. for 1 min before N-ethyl-N-isopropylpropan-2-amine (0.60 g, 4.6 mmol) was added. The reaction mixture was stirred at 60° C. for 2 h. The reaction mixture was cooled to ambient temperature and diluted with ethyl acetate (100 mL). The reaction mixture was washed with 1M sodium hydroxide (50 mL), and saturated ammonium chloride solution (2×50 mL). The organic layer was dried over anhydrous magnesium sulfate, filtered, and concentrated in vacuo. Purification of the residue by column chromatography, eluting with 5 to 50% of ethyl acetate in heptane, provided the title compound as a yellow solid (0.20 g, 91% yield): MS (ES+) m/z 465.0 (M+1), 467.2 (M+1).

### Step 2. Preparation of N-(2-(4,4-difluorocyclohexyl)-4-(2,5-difluorophenyl)pyridin-3-yl)-2-ethoxypyrimidine-5-carboxamide

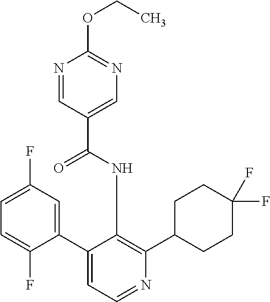 [SMILES: CCOc1ncc(C(=O)NCc2c(-c3cc(F)ccc3F)ccnc2C2CCC(F)(F)CC2)cn1]

To a mixture of 2-chloro-N-(2-(4,4-difluorocyclohexyl)-4-(2,5-difluorophenyl)pyridin-3-yl)pyrimidine-5-carboxamide (0.10 g, 0.22 mmol) in anhydrous ethanol (2.1 mL) was added 60% sodium hydride dispersion in mineral oil (0.021 g, 0.54 mmol). The solution was stirred at ambient temperature for 3 h. The reaction mixture was diluted with ethyl acetate (100 mL), washed with 1M sodium hydroxide (50 mL), and saturated ammonium chloride solution (2×50 mL). The organic layer was dried over anhydrous magnesium sulfate, filtered, and concentrated in vacuo. Purification of the residue by column chromatography, eluting with 10 to 60% of ethyl acetate in heptane, provided the title compound as a colorless solid (0.062 g, 60% yield): ¹H-NMR (400 MHz; DMSO-d₆) δ 10.22 (s, 1H), 8.87 (d, J=2.7 Hz, 2H), 8.63 (d, J=4.9 Hz, 1H), 7.38 (dd, J=4.9, 0.8 Hz, 1H), 7.35 (td, J=8.6, 4.0 Hz, 1H), 7.30-7.21 (m, 2H), 4.42 (q, J=7.1 Hz, 2H), 3.21-3.17 (m, 1H), 2.13-1.82 (m, 8H), 1.37-1.34 (m, 3H); MS (ES+) m/z 475.2 (M+1).

### Example 299

### Synthesis of N-(2-(4,4-difluorocyclohexyl)-4-(2,5-difluorophenyl)pyridin-3-yl)-6-ethoxynicotinamide

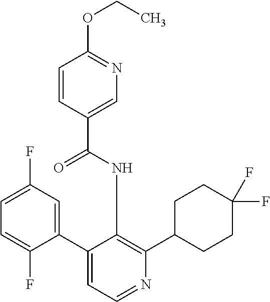 [SMILES: CCOc1ccc(C(=O)NCc2c(-c3cc(F)ccc3F)ccnc2C2CCC(F)(F)CC2)cn1]

To a mixture of 6-chloro-N-(2-(4,4-difluorocyclohexyl)-4-(2,5-difluorophenyl)pyridin-3-yl)nicotinamide (0.10 g, 0.22 mmol), anhydrous ethanol (0.10 mL), and 1,2-dioxane (0.84 mL) was added potassium tert-butoxide (0.19 g, 1.7 mmol). The solution was heated to 90° C. for 2 h. The reaction mixture was diluted with ethyl acetate (125 mL), and washed with 1M sodium hydroxide (50 mL), and saturated ammonium chloride solution (2×50 mL). The organic layer was dried over anhydrous magnesium sulfate, filtered, and concentrated in vacuo. Purification of the residue by column chromatography, eluting with 10 to 60% of ethyl acetate in heptane, provided the title compound as a colorless solid (0.062 g, 60% yield): ¹H-NMR (400 MHz; DMSO-d₆) δ 10.05 (s, 1H), 8.61 (d, J=4.9 Hz, 1H), 8.55 (dd, J=2.5, 0.6 Hz, 1H), 7.98 (dd, J=8.7, 2.5 Hz, 1H), 7.36 (dd, J=4.9, 0.8 Hz, 1H), 7.33 (td, J=8.9, 4.3 Hz, 1H), 7.28-7.20 (m, 2H), 6.87 (dd, J=8.7, 0.6 Hz, 1H), 4.36 (q, J=7.0 Hz, 2H), 3.19-3.17 (m, 1H), 2.14-2.07 (m, 2H), 1.94-1.81 (m, 6H), 1.33 (t, J=7.0 Hz, 3H); MS (ES+) m/z 474.2 (M+1).

### Example 300 and 301

### Synthesis of N-(4-(2,5-difluorophenyl)-2-(4-fluorocyclohexyl)pyridin-3-yl)-2-isopropoxypyrimidine-5-carboxamide P1 and P2

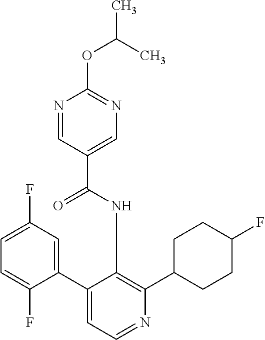 [SMILES: CC(C)Oc1ncc(C(=O)NCc2c(-c3cc(F)ccc3F)ccnc2C2CCC(F)CC2)cn1]

### Step 1. Preparation of 2-chloro-N-(4-(2,5-difluorophenyl)-2-(4-fluorocyclohexyl)pyridin-3-yl)pyrimidine-5-carboxamide

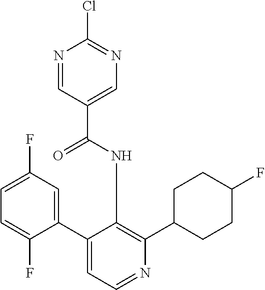 [SMILES: O=C(NCc1c(-c2cc(F)ccc2F)ccnc1C1CCC(F)CC1)c1cnc(Cl)nc1]

To a mixture of 4-(2,5-difluorophenyl)-2-(4-fluorocyclohexyl)pyridin-3-amine (0.15 g, 0.49 mmol), 2-chloropyrimidine-5-carboxylic acid (0.10 g, 0.64 mmol) and 2-chloro-1-methylpyridinium iodide (0.34 g, 1.3 mmol) was added anhydrous tetrahydrofuran (10 mL). The solution was heated at 60° C. for 1 min before N-ethyl-N-isopropylpropan-2-amine (0.63 g, 4.9 mmol) was added. The reaction mixture was stirred at 60° C. for 7 h. The reaction mixture was cooled to ambient temperature and diluted with ethyl acetate (125 mL). The reaction mixture was washed with saturated ammonium chloride solution (2×50 mL). The organic layer was dried over anhydrous magnesium sulfate, filtered, and concentrated in vacuo. Purification of the residue by column chromatography, eluting with 5 to 60% of ethyl acetate in heptane, provided the title compound as a colorless solid (0.076 g, 35% yield): MS (ES+) m/z 447.2 (M+1), 449.0 (M+1).

### Step 2. Preparation of N-(4-(2,5-difluorophenyl)-2-(4-fluorocyclohexyl)pyridin-3-yl)-2-isopropoxypyrimidine-5-carboxamide P1 and P2

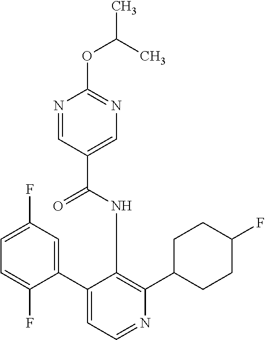 [SMILES: CC(C)Oc1ncc(C(=O)NCc2c(-c3cc(F)ccc3F)ccnc2C2CCC(F)CC2)cn1]

To a vial containing 2-chloro-N-(4-(2,5-difluorophenyl)-2-(4-fluorocyclohexyl)pyridin-3-yl)pyrimidine-5-carboxamide (0.23 g, 0.54 mmol) was added anhydrous N,N-dimethylformamide (1.1 mL), 2-propanol (1.3 mL), and 60% sodium hydride dispersion in mineral oil (0.041 g, 1.7 mmol). The reaction vessel was sealed and heated to 40° C. for 2 h. The reaction mixture was cooled to ambient temperature, diluted with ethyl acetate (100 mL) and washed with 1M sodium hydroxide (50 mL), and saturated ammonium chloride solution (2×50 mL). The organic layer was dried over anhydrous magnesium sulfate, filtered, and concentrated in vacuo. Purification of the residue by column chromatography, eluting with 10 to 70% of ethyl acetate in heptane followed by reverse phase column chromatography, eluting with 30 to 90% acetonitrile in water with 0.5% formic acid, provided the title compounds as colorless solid N-(4-(2,5-difluorophenyl)-2-(4-fluorocyclohexyl)pyridin-3-yl)-2-isopropoxypyrimidine-5-carboxamide P1 (0.024 g, 29% yield): ¹H-NMR (400 MHz; DMSO-d₆) δ 10.19 (s, 1H), 8.86 (s, 2H), 8.63 (d, J=4.9 Hz, 1H), 7.37-7.31 (m, 2H), 7.29-7.21 (m, 2H), 5.27 (quintet, J=6.2 Hz, 1H), 4.94-4.81 (m, 1H), 3.10-3.10 (m, 1H), 2.04-1.92 (m, 4H), 1.67-1.55 (m, 4H), 1.34 (d, J=6.2 Hz, 6H); MS (ES+) m/z 471.0 (M+1). N-(4-(2,5-difluorophenyl)-2-(4-fluorocyclohexyl)pyridin-3-yl)-2-isopropoxypyrimidine-5-carboxamide P2 (0.026 g, 32% yield): ¹H-NMR (400 MHz; DMSO-d₆) δ 10.20 (s, 1H), 8.86 (d, J=3.3 Hz, 2H), 8.60 (d, J=4.9 Hz, 1H), 7.34 (dq, J=9.1, 4.5 Hz, 2H), 7.29-7.21 (m, 2H), 5.27 (quintet, J=6.2 Hz, 1H), 4.68-4.55 (m, 1H), 3.06-3.00 (m, 1H), 2.13-2.09 (m, 2H), 1.85-1.70 (m, 4H), 1.56-1.47 (m, 2H), 1.33 (t, J=7.1 Hz, 6H); MS (ES+) m/z 471.0 (M+1).

### Example 302

### Synthesis of N-(2-(4,4-difluorocyclohexyl)-4-(2,5-difluorophenyl)pyridin-3-yl)-6-ethoxy-5-fluoronicotinamide

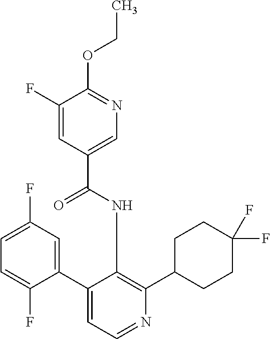 [SMILES: CCOc1ncc(C(=O)NCc2c(-c3cc(F)ccc3F)ccnc2C2CCC(F)(F)CC2)cc1F]

To a mixture of N-(2-(4,4-difluorocyclohexyl)-4-(2,5-difluorophenyl)pyridin-3-yl)-5,6-difluoronicotinamide (0.048 g, 0.10 mmol) and anhydrous ethanol (1.0 mL) was added 60% sodium hydride dispersion in mineral oil (0.008 g, 0.2 mmol). The mixture was heated to 50° C. for 4 h. The reaction mixture was diluted with ethyl acetate (125 mL), and washed with 1M sodium hydroxide (50 mL), and saturated ammonium chloride solution (2×50 mL). The organic layer was dried over anhydrous magnesium sulfate, filtered, and concentrated in vacuo. Purification of the residue by column chromatography, eluting with 10 to 70% of ethyl acetate in heptane, provided the title compound as a colorless solid (0.025 g, 45% yield): ¹H-NMR (400 MHz; DMSO-d₆) S 10.13 (s, 1H), 8.62 (t, J=4.3 Hz, 1H), 8.39-8.37 (m, 1H), 7.92 (dd, J=11.0, 2.0 Hz, 1H), 7.40-7.31 (m, 2H), 7.29-7.20 (m, 2H), 4.47 (q, J=7.0 Hz, 2H), 3.20-3.14 (m, 1H), 2.14-2.05 (m, 2H), 1.99-1.80 (m, 6H), 1.39-1.35 (m, 3H); MS (ES+) m/z 492.0 (M+1).

### Example 303

### Synthesis of N-(2-(4,4-difluorocyclohexyl)-4-(2,5-difluorophenyl)pyridin-3-yl)-2-(isoxazolidin-2-yl)pyrimidine-5-carboxamide

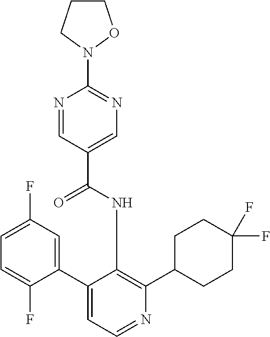 [SMILES: O=C(NCc1c(-c2cc(F)ccc2F)ccnc1C1CCC(F)(F)CC1)c1cnc(N2CCCO2)nc1]

### Step 1. Preparation of 2-chloro-N-(2-chloropyrimidine-5-carbonyl)-N-(2-(4,4-difluorocyclohexyl)-4-(2,5-difluorophenyl)pyridin-3-yl)pyrimidine-5-carboxamide

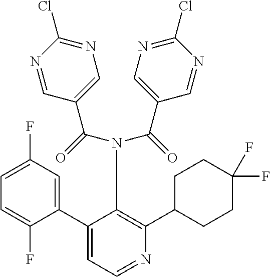 [SMILES: O=C(c1cnc(Cl)nc1)N(Cc1c(-c2cc(F)ccc2F)ccnc1C1CCC(F)(F)CC1)C(=O)c1cnc(Cl)nc1]

To a mixture of 2-(4,4-difluorocyclohexyl)-4-(2,5-difluorophenyl)pyridin-3-amine (0.25 g, 0.77 mmol), 2-chloropyrimidine-5-carboxylic acid (0.18 g, 1.2 mmol) and 2-chloro-1-methylpyridinium iodide (0.53 g, 2.1 mmol) was added anhydrous tetrahydrofuran (15 mL) and N-ethyl-N-isopropylpropan-2-amine (1.0 g, 7.7 mmol). The reaction mixture was stirred at 50° C. for 18 h. The reaction mixture was cooled to 5° C. and diluted with 2-propanol (2 mL), and 10M sodium hydroxide (1 mL). After 1 h the reaction mixture was diluted with ethyl acetate (100 mL). The reaction mixture was washed with 1M sodium hydroxide (50 mL) and saturated ammonium chloride solution (2×50 mL). The organic layer was dried over anhydrous magnesium sulfate, filtered and concentrated in vacuo. Purification of the residue by column chromatography, eluting with 5 to 50% of ethyl acetate in heptane, provided the title compound as a yellow solid (0.36 g, 77% yield): MS (ES+) m/z 605.2 (M+1), 606.2 (M+1), 607.2 (M+1).

### Step 2. Preparation of N-(2-(4,4-difluorocyclohexyl)-4-(2,5-difluorophenyl)pyridin-3-yl)-2-(isoxazolidin-2-yl)pyrimidine-5-carboxamide

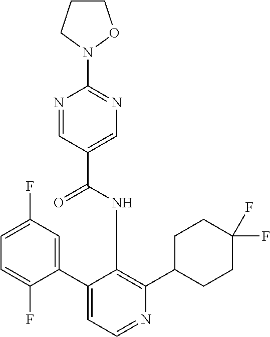 [SMILES: O=C(NCc1c(-c2cc(F)ccc2F)ccnc1C1CCC(F)(F)CC1)c1cnc(N2CCCO2)nc1]

To a mixture of 2-chloro-N-(2-chloropyrimidine-5-carbonyl)-N-(2-(4,4-difluorocyclohexyl)-4-(2,5-difluorophenyl)pyridin-3-yl)pyrimidine-5-carboxamide (0.054 g, 0.089 mmol), anhydrous N,N-dimethylformamide (1.0 mL), and isoxazolidine hydrochloride (0.019 g, 0.18 mmol) was added potassium tert-butoxide (0.049 g, 0.36 mmol). The solution was heated to 40° C. for 1 h. The reaction mixture was diluted with methanol (2 mL) and 50% sodium hydroxide (1 mL) and stirred for 1 h. The reaction mixture was diluted with ethyl acetate (100 mL), and washed with 1M sodium hydroxide (50 mL), and saturated ammonium chloride solution (2×50 mL). The organic layer was dried over anhydrous magnesium sulfate, filtered and concentrated in vacuo. Purification of the residue by column chromatography, eluting with 10 to 100% of ethyl acetate in heptane, provided the title compound as a colorless solid (0.062 g, 60% yield): ¹H-NMR (400 MHz; DMSO-d₆) δ 10.08 (s, 1H), 8.76 (s, 2H), 8.61 (d, J=4.9 Hz, 1H), 7.37 (dd, J=4.9, 0.8 Hz, 1H), 7.34 (dt, J=8.7, 4.1 Hz, 1H), 7.30-7.20 (m, 2H), 3.95 (dd, J=8.9, 5.0 Hz, 2H), 3.85 (dd, J=8.6, 6.1 Hz, 2H), 3.19-3.16 (m, 1H), 2.31-2.24 (m, 2H), 2.13-2.06 (m, 2H), 1.99-1.80 (m, 6H); MS (ES+) m/z 502.0 (M+1).

### Example 304

### Synthesis of rac-N-(2-(4,4-difluorocyclohexyl)-4-(2,5-difluorophenyl)pyridin-3-yl)-6-(5,5-difluorotetrahydro-2H-pyran-2-yl)nicotinamide

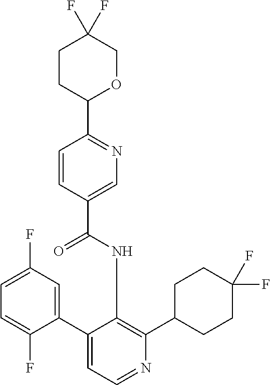 [SMILES: O=C(NCc1c(-c2cc(F)ccc2F)ccnc1C1CCC(F)(F)CC1)c1ccc(C2CCC(F)(F)CO2)nc1]

To 6-chloro-N-(2-(4,4-difluorocyclohexyl)-4-(2,5-difluorophenyl)pyridin-3-yl)nicotinamide (0.050 g, 0.11 mmol) was added 5,5-difluorotetrahydro-2H-pyran-2-carboxylic acid (0.046 g, 0.27 mmol), (4,4″-di-t-butyl-2,2″-bipyridine)bis[3,5-difluoro-2-[5-trifluoromethyl-2-pyridinyl-kN)phenyl-kC]iridium(III) hexafluorophosphate (0.0018 g, 0.0016 mmol), dichloro(dimethoxyethane)nickel (0.0035 g, 0.016 mmol), 4-tert-butyl-2-(4-tert-butyl-2-pyridyl)pyridine (0.0065 g, 0.024 mmol), cesium carbonate (0.095 g, 0.29 mmol), and N,N-dimethylformamide (2.7 mL). The headspace of the vial was replaced with nitrogen, the vial was sealed, and the reaction mixture was stirred in front of Kessil PR160L lights (440 nm) for 48 h. The reaction mixture was diluted with ethyl acetate (100 mL) and washed with 1M sodium hydroxide (25 mL), saturated ammonium chloride solution (2×25 mL), dried over anhydrous magnesium sulfate, filtered, and concentrated in vacuo. Purification of the residue by column chromatography, eluting with 10 to 60% of ethyl acetate in heptane, afforded the title compound as a colorless solid (0.27 g, 45% yield): ¹H-NMR (400 MHz; DMSO-d₆) S 10.28 (s, 1H), 8.83 (dd, J=2.2, 0.7 Hz, 1H), 8.62 (d, J=4.9 Hz, 1H), 8.10 (dd, J=8.2, 2.3 Hz, 1H), 7.57 (d, J=8.2 Hz, 1H), 7.38 (dd, J=4.9, 0.7 Hz, 1H), 7.35-7.31 (m, 1H), 7.28-7.21 (m, 2H), 4.73-4.70 (m, 1H), 4.10-4.04 (m, 1H), 3.93-3.82 (m, 1H), 3.22-3.17 (m, 1H), 2.31-2.21 (m, 3H), 2.14-2.06 (m, 2H), 1.99-1.71 (m, 7H); MS (ES+) m/z 550.2 (M+1).

### Example 305

### Synthesis of N-(2-(4,4-difluorocyclohexyl)-4-(2,5-difluorophenyl)pyridin-3-yl)-2-(2,2-difluoroethoxy)pyrimidine-5-carboxamide

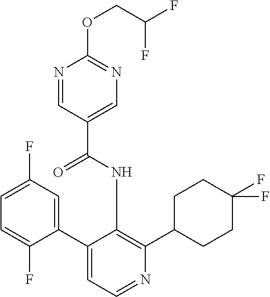 [SMILES: O=C(Nc1c(-c2cc(F)ccc2F)ccnc1C1CCC(F)(F)CC1)c1cnc(OCC(F)F)nc1]

To a mixture of 2-chloro-N-(2-chloropyrimidine-5-carbonyl)-N-(2-(4,4-difluorocyclohexyl)-4-(2,5-difluorophenyl)pyridin-3-yl)pyrimidine-5-carboxamide (0.087 g, 0.14 mmol), anhydrous N,N-dimethylformamide (1.4 mL), and 2,2-difluoroethanol (1.4 mL) was added 60% sodium hydride dispersion in mineral oil (0.010 g, 0.43 mmol). The mixture was stirred at ambient temperature for 1 h. The reaction was diluted with methanol (1 mL) and ethyl acetate (100 mL), and washed with 1M sodium hydroxide (2×50 mL), and saturated ammonium chloride solution (2×50 mL). The organic layer was dried over anhydrous magnesium sulfate, filtered, and concentrated in vacuo. Purification of the residue by column chromatography, eluting with 10 to 50% of ethyl acetate in heptane, provided the title compound as a colorless solid (0.062 g, 60% yield): ¹H-NMR (400 MHz; DMSO-d₆) δ 10.29 (s, 1H), 8.92 (d, J=3.2 Hz, 2H), 8.63 (d, J=4.9 Hz, 1H), 7.39-7.38 (m, 1H), 7.38-7.32 (m, 1H), 7.30-7.22 (m, 2H), 6.44 (tt, J=54.3, 3.4 Hz, 1H), 4.71 (td, J=15.0, 3.4 Hz, 2H), 3.23-3.17 (m, 1H), 2.14-2.05 (m, 2H), 2.03-1.81 (m, 6H); MS (ES+) m/z 511.2 (M+1).

### Example 306

### Synthesis of rac-N-(4-(2,5-difluorophenyl)-2-(5,5-difluorotetrahydro-2H-pyran-2-yl) pyridin-3-yl)-2-ethoxypyrimidine-5-carboxamide

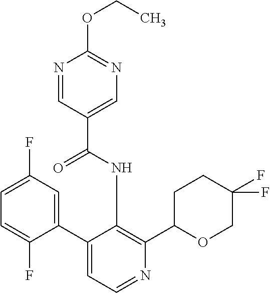 [SMILES: CCOc1ncc(C(=O)Nc2c(-c3cc(F)ccc3F)ccnc2C2CCC(F)(F)CO2)cn1]

To a mixture of rac-2-chloro-N-(2-chloropyrimidine-5-carbonyl)-N-(4-(2,5-difluorophenyl)-2-(5,5-difluorotetrahydro-2H-pyran-2-yl)pyridin-3-yl)pyrimidine-5-carboxamide (0.15 g, 0.25 mmol) in ethanol (2.5 mL) and 60% sodium hydride dispersion in mineral oil (0.049 g, 1.2 mmol). The mixture was heated to 45° C. for 18 h. The reaction mixture was diluted with ethyl acetate (100 mL) and washed with 1M sodium hydroxide (50 mL), and saturated ammonium chloride solution (2×50 mL). The organic layer was dried over anhydrous magnesium sulfate, filtered and concentrated in vacuo. Purification of the residue by column chromatography, eluting with 5 to 60% of ethyl acetate in heptane, provided the title compound as a colorless solid (0.083 g, 70% yield): ¹H-NMR (400 MHz; DMSO-d₆) δ 10.24 (s, 1H), 8.90-8.87 (m, 2H), 8.67 (d, J=4.9 Hz, 1H), 7.53 (dd, J=4.9, 0.8 Hz, 1H), 7.39-7.32 (m, 1H), 7.31-7.25 (m, 2H), 4.93-4.90 (m, 1H), 4.45-4.40 (m, 2H), 3.95-3.88 (m, 1H), 3.84-3.73 (m, 1H), 2.32-2.15 (m, 3H), 1.99-1.93 (m, 1H), 1.35 (dd, J=9.1, 5.0 Hz, 3H); MS (ES+) m/z 477.2 (M+1).

### Example 307

### Synthesis of rac-N-(4-(2,5-difluorophenyl)-2-(5,5-difluorotetrahydro-2H-pyran-2-yl)pyridin-3-yl)-2-(isopropylamino)pyrimidine-5-carboxamide

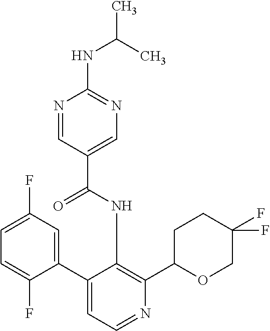 [SMILES: CC(C)Nc1ncc(C(=O)Nc2c(-c3cc(F)ccc3F)ccnc2C2CCC(F)(F)CO2)cn1]

To a mixture of rac-2-chloro-N-(2-chloropyrimidine-5-carbonyl)-N-(4-(2,5-difluorophenyl)-2-(5,5-difluorotetrahydro-2H-pyran-2-yl)pyridin-3-yl)pyrimidine-5-carboxamide (0.13 g, 0.21 mmol) 2-aminopropane (0.062 g, 1.0 mmol), and anhydrous N,N-dimethylformamide (2.1 mL) was added N,N-diisopropylethylamine (0.36 mL). The solution was heated to 60° C. for 6 h. The reaction mixture was diluted with ethyl acetate (125 mL) and washed with 1M sodium hydroxide (50 mL), and saturated ammonium chloride solution (2×50 mL). The organic layer was dried over anhydrous magnesium sulfate, filtered, and concentrated in vacuo. Purification of the residue by column chromatography, eluting with 10 to 100% of ethyl acetate in heptane, provided the title compound as a colorless solid (0.054 g, 52% yield): ¹H-NMR (400 MHz; DMSO-d₆) δ 9.83 (s, 1H), 8.64 (d, J=4.8 Hz, 2H), 8.58 (br s, 1H), 7.79 (d, J=8.0 Hz, 1H), 7.49 (dd, J=4.9, 0.9 Hz, 1H), 7.34 (td, J=8.8, 4.4 Hz, 1H), 7.30-7.23 (m, 2H), 4.89-4.86 (m, 1H), 4.14-4.05 (m, 1H), 3.96-3.89 (m, 1H), 3.77 (dt, J=20.0, 12.0 Hz, 1H), 2.34-2.13 (m, 3H), 1.97-1.92 (m, 1H), 1.15 (d, J=6.4 Hz, 6H); MS (ES+) m/z 490.2 (M+1).

### Example 308

### Synthesis of N-(2′-(4,4-difluorocyclohexyl)-3-fluoro-[2,4′-bipyridin]-3′-yl)-5-fluoro-6-isopropoxynicotinamide

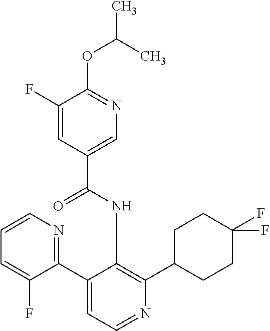 [SMILES: CC(C)Oc1ncc(C(=O)Nc2c(-c3ncccc3F)ccnc2C2CCC(F)(F)CC2)cc1F]

### Step 1. Preparation of N-(2′-(4,4-difluorocyclohexyl)-3-fluoro-[2,4′-bipyridin]-3′-yl)-5,6-difluoronicotinamide

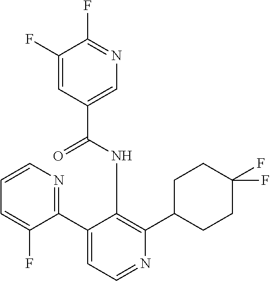 [SMILES: O=C(Nc1c(-c2ncccc2F)ccnc1C1CCC(F)(F)CC1)c1cnc(F)c(F)c1]

To a mixture of 2′-(4,4-difluorocyclohexyl)-3-fluoro-[2,4′-bipyridin]-3′-amine (0.075 g, 0.24 mmol), 2,3-difluoropyridine-5-carboxylic acid (0.058 g, 0.37 mmol) and 2-chloro-1-methylpyridinium iodide (0.17 g, 0.68 mmol) was added anhydrous tetrahydrofuran (4.9 mL) and N-ethyl-N-isopropylpropan-2-amine (0.32 g, 2.4 mmol). The reaction mixture was stirred at ambient temperature for 18 h. The reaction mixture was diluted with ethyl acetate (100 mL). The reaction mixture was washed with saturated ammonium chloride solution (2×50 mL). The organic layer was dried over anhydrous magnesium sulfate, filtered, and concentrated in vacuo. Purification of the residue by column chromatography, eluting with 0 to 80% of ethyl acetate in heptane, provided the title compound as a colorless oil (0.085 g, 78% yield): MS (ES+) m/z 449.4 (M+1).

### Step 2. Preparation of N-(2′-(4,4-difluorocyclohexyl)-3-fluoro-[2,4′-bipyridin]-3′-yl)-5-fluoro-6-isopropoxynicotinamide

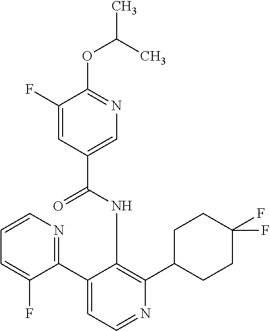 [SMILES: CC(C)Oc1ncc(C(=O)Nc2c(-c3ncccc3F)ccnc2C2CCC(F)(F)CC2)cc1F]

To a mixture of N-(2′-(4,4-difluorocyclohexyl)-3-fluoro-[2,4′-bipyridin]-3′-yl)-5,6-difluoronicotinamide (0.085 g, 0.19 mmol), anhydrous N,N-dimethylformamide (1.0 mL), and anhydrous 2-propanol (1.0 mL) was added 60% sodium hydride dispersion in mineral oil (0.005 g, 0.2 mmol). The solution was heated to 50° C. for 2 h. The reaction mixture was diluted with ethyl acetate (125 mL) then washed with 1M sodium hydroxide (50 mL), and saturated ammonium chloride solution (2×50 mL). The organic layer was dried over anhydrous magnesium sulfate, filtered, and concentrated in vacuo. Purification of the residue by column chromatography, eluting with 10 to 80% of ethyl acetate in heptane followed by reverse phase column chromatography, eluting with a gradient of 5 to 95% of acetonitrile in water, provided the title compound as a colorless solid (0.028 g, 30% yield): ¹H-NMR (400 MHz; DMSO-d₆) δ 10.17-10.13 (m, 1H), 8.65 (d, J=4.9 Hz, 1H), 8.45 (dt, J=4.5, 1.4 Hz, 1H), 8.36 (d, J=2.0 Hz, 1H), 7.89 (dd, J=11.1, 2.0 Hz, 1H), 7.81 (ddd, J=10.1, 8.6, 1.3 Hz, 1H), 7.50-7.45 (m, 2H), 5.38 (dt, J=12.4, 6.2 Hz, 1H), 3.21-3.15 (m, 1H), 2.14-2.06 (m, 2H), 1.95-1.83 (m, 6H), 1.36-1.32 (m, 6H); MS (ES+) m/z 489.2 (M+1).

### Example 309

### Synthesis of 2-cyclobutoxy-N-(2′-(4,4-difluorocyclohexyl)-3-fluoro-[2,4′-bipyridin]-3′-yl)pyrimidine-5-carboxamide

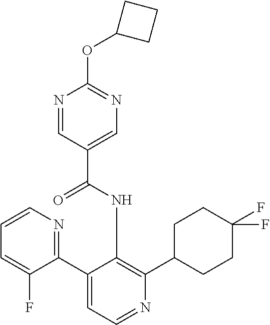 [SMILES: O=C(Nc1c(-c2ncccc2F)ccnc1C1CCC(F)(F)CC1)c1cnc(OC2CCC2)nc1]

### Step 1. Preparation of 2-chloro-N-(2-chloropyrimidine-5-carbonyl)-N-(2′-(4,4-difluorocyclohexyl)-3-fluoro-[2,4′-bipyridin]-3′-yl)pyrimidine-5-carboxamide

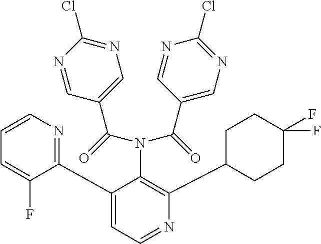 [SMILES: O=C(c1cnc(Cl)nc1)N(C(=O)c1cnc(Cl)nc1)c1c(-c2ncccc2F)ccnc1C1CCC(F)(F)CC1]

To a mixture of 2′-(4,4-difluorocyclohexyl)-3-fluoro-[2,4′-bipyridin]-3′-amine (0.084 g, 0.27 mmol), 2-chloropyrimidine-5-carboxylic acid (0.052 g, 0.33 mmol) and 2-chloro-1-methylpyridinium iodide (0.14 g, 0.55 mmol) was added anhydrous tetrahydrofuran (5.5 mL) and N-ethyl-N-isopropylpropan-2-amine (0.35 g, 2.7 mmol). The reaction mixture was stirred at ambient temperature for 18 h. The reaction mixture was diluted with ethyl acetate (100 mL). The reaction mixture was washed with 1M sodium hydroxide (50 mL), and saturated ammonium chloride solution (2×50 mL). The organic layer was dried over anhydrous magnesium sulfate, filtered, and concentrated in vacuo. Purification of the residue by column chromatography, eluting with 10 to 75% of ethyl acetate in heptane, provided the title compound as a colorless oil (0.085 g, 78% yield): MS (ES+) m/z 588.4 (M+1), 589.4 (M+1), 590.4 (M+1).

### Step 2. Preparation of 2-cyclobutoxy-N-(2′-(4,4-difluorocyclohexyl)-3-fluoro-[2,4′-bipyridin]-3′-yl)pyrimidine-5-carboxamide

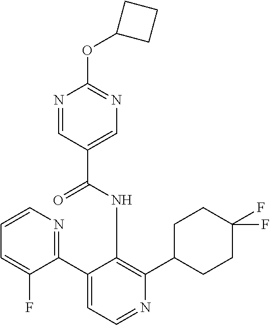 [SMILES: O=C(Nc1c(-c2ncccc2F)ccnc1C1CCC(F)(F)CC1)c1cnc(OC2CCC2)nc1]

To a mixture of 2-chloro-N-(2-chloropyrimidine-5-carbonyl)-N-(2′-(4,4-difluorocyclohexyl)-3-fluoro-[2,4′-bipyridin]-3′-yl)pyrimidine-5-carboxamide (0.061 g, 0.10 mmol), anhydrous N,N-dimethylformamide (0.50 mL), and cyclobutanol (0.37 g, 5.1 mmol) was added 60% sodium hydride dispersion in mineral oil (0.012 g, 0.51 mmol). The solution was heated to 50° C. for 2 h. The reaction mixture was diluted with ethyl acetate (125 mL) and washed with 1M sodium hydroxide (50 mL), and saturated ammonium chloride solution (2×50 mL). The organic layer was dried over anhydrous magnesium sulfate, filtered, and concentrated in vacuo. Purification of the residue by column chromatography, eluting with 5 to 100% of ethyl acetate in heptane followed by reverse phase column chromatography, eluting with a gradient of 5 to 95% acetonitrile in water, provided the title compound as a colorless solid (0.028 g, 30% yield): ¹H-NMR (400 MHz; DMSO-d₆) δ 10.24 (s, 1H), 8.83 (s, 2H), 8.65 (d, J=4.9 Hz, 1H), 8.45 (dt, J=4.6, 1.5 Hz, 1H), 7.82 (ddd, J=10.1, 8.6, 1.3 Hz, 1H), 7.50 (dd, J=8.6, 4.2 Hz, 1H), 7.46 (dd, J=4.9, 1.3 Hz, 1H), 5.18 (t, J=7.4 Hz, 1H), 3.21-3.19 (m, 1H), 2.46-2.38 (m, 2H), 2.14-2.06 (m, 4H), 1.98-1.79 (m, 7H), 1.70-1.64 (m, 1H); MS (ES+) m/z 484.2 (M+1).

### Example 310

### Synthesis of N-(2-(4,4-difluorocyclohexyl)-4-(2,5-difluorophenyl)pyridin-3-yl)-2-isopropoxy-N-methylpyrimidine-5-carboxamide

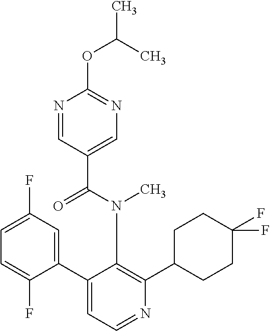 [SMILES: CC(C)Oc1ncc(C(=O)N(C)c2c(-c3cc(F)ccc3F)ccnc2C2CCC(F)(F)CC2)cn1]

To a mixture of N-(2-(4,4-difluorocyclohexyl)-4-(2,5-difluorophenyl)pyridin-3-yl)-2-isopropoxypyrimidine-5-carboxamide (0.10 g, 0.20 mmol) and anhydrous N,N-dimethylformamide (2.0 mL) was added a 60% sodium hydride dispersion in mineral oil (0.025 g, 0.61 mmol). The reaction mixture was stirred at ambient temperature for 30 min before methyl iodide (0.087 g, 0.038 mmol) was added. The reaction mixture was stirred at ambient temperature for 18 h. The reaction mixture was diluted with ethyl acetate (100 mL) and washed with saturated ammonium chloride solution (2×50 mL). The organic layer was dried over anhydrous magnesium sulfate, filtered, and concentrated in vacuo. Purification of the residue by column chromatography, eluting with 10 to 60% of ethyl acetate in heptane, followed by reverse phase column chromatography, eluting with a gradient of 10 to 90% acetonitrile in water, afforded the title compound as a colorless solid (0.052 g, 51% yield): ¹H-NMR (400 MHz; DMSO-d₆, 2:1 mixture of rotamers) δ 8.67 (t, J=4.7 Hz, 3H), 8.53 (s, 2H), 7.81 (s, 4H), 7.49-7.35 (m, 5H), 7.32-7.24 (m, 4H), 7.19-7.16 (m, 1H), 6.93-6.89 (m, 2H), 5.26 (t, J=6.2 Hz, 1H), 5.14 (quintet, J=6.2 Hz, 2H), 3.36-3.27 (m, 13H), 2.21-1.81 (m, 22H), 1.66-1.62 (m, 2H), 1.34 (d, J=6.2 Hz, 5H), 1.26 (t, J=6.7 Hz, 12H); MS (ES+) m/z 503.2 (M+1).

### Example 311

### Synthesis of 2-(tert-butoxy)-N-(2-(4,4-difluorocyclohexyl)-4-(2,5-difluorophenyl)pyridin-3-yl)pyrimidine-5-carboxamide

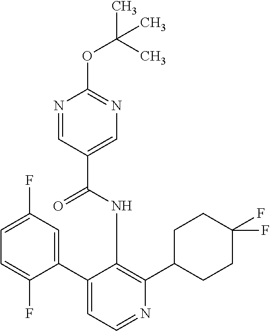 [SMILES: CC(C)(C)Oc1ncc(C(=O)Nc2c(-c3cc(F)ccc3F)ccnc2C2CCC(F)(F)CC2)cn1]

### Step 1. Preparation of 2-cyano-N-(2-(4,4-difluorocyclohexyl)-4-(2,5-difluorophenyl)pyridin-3-yl)pyrimidine-5-carboxamide

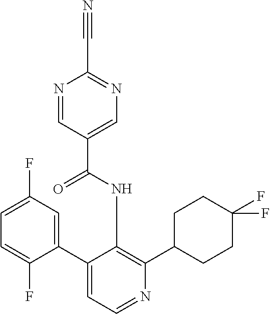 [SMILES: N#Cc1ncc(C(=O)Nc2c(-c3cc(F)ccc3F)ccnc2C2CCC(F)(F)CC2)cn1]

To a mixture of 2-(4,4-difluorocyclohexyl)-4-(2,5-difluorophenyl)pyridin-3-amine (0.13 g, 0.40 mmol), 2-cyanopyrimidine-5-carboxylic acid (0.90 g, 0.60 mmol) and 2-chloro-1-methylpyridinium iodide (0.52 g, 0.40 mmol) was added anhydrous tetrahydrofuran (8.0 mL). The solution was heated at 55° C. for 1 min before N-ethyl-N-isopropylpropan-2-amine (0.31 g, 1.2 mmol) was added. The reaction mixture was stirred at 55° C. for 2 h. The reaction mixture was cooled to ambient temperature and diluted with methanol (1 mL), and ethyl acetate (150 mL). The reaction mixture was washed with 1M sodium hydroxide (20 mL), saturated ammonium chloride solution (2×50 mL). The organic layer was dried over anhydrous magnesium sulfate, filtered and concentrated in vacuo. Purification of the residue by column chromatography, eluting with 10 to 75% of ethyl acetate in heptane, provided the title compound as a colorless oil (0.18 g, 99% yield): MS (ES+) m/z 456.4 (M+1).

### Step 2. Preparation of 2-(tert-butoxy)-N-(2-(4,4-difluorocyclohexyl)-4-(2,5-difluorophenyl)pyridin-3-yl)pyrimidine-5-carboxamide

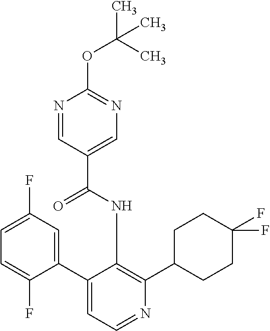 [SMILES: CC(C)(C)Oc1ncc(C(=O)Nc2c(-c3cc(F)ccc3F)ccnc2C2CCC(F)(F)CC2)cn1]

To a mixture of 2-cyano-N-(2-(4,4-difluorocyclohexyl)-4-(2,5-difluorophenyl)pyridin-3-yl)pyrimidine-5-carboxamide (0.18 g, 0.40 mmol), tert-butanol (0.10 mL) and anhydrous N,N-dimethylformamide (1.2 mL) was added potassium tert-butoxide (0.045 g, 0.40 mmol). The reaction mixture was sealed and heated to 85° C. for 18 h. The reaction mixture was diluted with ethyl acetate (150 mL) and washed with saturated ammonium chloride solution (2×50 mL). The organic layer was dried over anhydrous magnesium sulfate, filtered, and concentrated in vacuo. Purification of the residue by column chromatography, eluting with 10 to 60% of ethyl acetate in heptane, afforded the title compound as a colorless solid (0.034 g, 16% yield): ¹H-NMR (400 MHz; DMSO-d₆) δ 10.18 (s, 1H), 8.83 (s, 2H), 8.62 (d, J=4.9 Hz, 1H), 7.38-7.32 (m, 2H), 7.29-7.22 (m, 2H), 3.21-3.19 (m, 1H), 2.11-2.06 (m, 2H), 1.99-1.82 (m, 6H), 1.59 (s, 9H); MS (ES+) m/z 503.3 (M+1).

### Example 312

In a similar manner as described in examples disclosed herein, utilizing the appropriately substituted starting materials and intermediates, the following compounds were prepared:

| Example | Structure | Amount | MS (ES+) | |
|---|---|---|---|---|
| No. | Name | Yield | m/z | ¹H NMR |
| 312 | 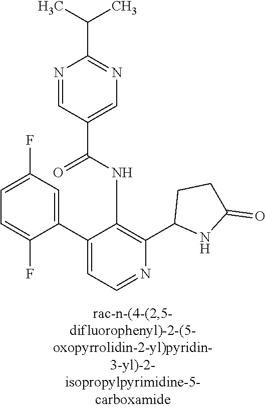 [SMILES: CC(C)c1ncc(C(=O)Nc2c(-c3cc(F)ccc3F)ccnc2C2CCC(=O)N2)cn1] | 0.074 g 63% | 438.2 (M + 1) | (400 MHz, DMSO-d₆) δ 10.43 (d, J = 0.4 Hz, 1H), 8.97 (s, 2H), 8.68 (d, J = 4.9 Hz, 1H), 7.90-7.89 (m, 1H), 7.48 (d, J = 4.8 Hz, 1H), 7.38 (td, J = 9.2, 4.6 Hz, 1H), 7.33-7.24 (m, 2H), 5.07 (dd, J = 8.1, 3.9 Hz, 1H), 3.20 (dt, J = 13.8, 6.9 Hz, 1H), 2.32- 2.24 (m, 1H), 2.16 (dt, J = 10.4, 5.4 Hz, 1H), 2.01-1.96 (m, 1H), 1.29 (d, J = 6.9 Hz, 6H) |

### Example 313

### Synthesis of rac-N-(2′-(5,5-difluorotetrahydro-2H-pyran-2-yl)-3-fluoro-[2,4′-bipyridin]-3′-yl)-5-fluoro-6-isopropoxynicotinamide

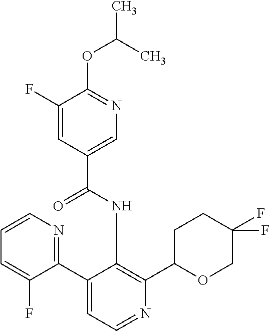 [SMILES: CC(C)Oc1ncc(C(=O)Nc2c(-c3ncccc3F)ccnc2C2CCC(F)(F)CO2)cc1F]

### Step 1. Preparation of rac-tert-butyl (2′-(5,5-difluorotetrahydro-2H-pyran-2-yl)-3-fluoro-[2,4′-bipyridin]-3′-yl)carbamate

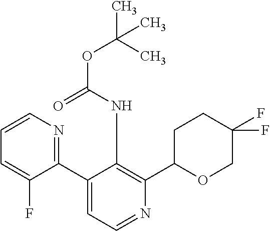 [SMILES: CC(C)(C)OC(=O)Nc1c(-c2ncccc2F)ccnc1C1CCC(F)(F)CO1]

To tert-butyl (2′-chloro-3-fluoro-[2,4′-bipyridin]-3′-yl)carbamate (0.80 g, 2.5 mmol) was added 5,5-difluorotetrahydro-2H-pyran-2-carboxylic acid (0.62 g, 3.7 mmol), (4,4″-di-t-butyl-2,2″-bipyridine)bis[3,5-difluoro-2-[5-trifluoromethyl-2-pyridinyl-kN)phenyl-kC]iridium(III) hexafluorophosphate (0.028 g, 0.025 mmol), dichloro(dimethoxyethane)nickel (0.054 g, 0.25 mmol), 4-tert-butyl-2-(4-tert-butyl-2-pyridyl)pyridine (0.099 g, 0.37 mmol), cesium carbonate (1.4 g, 4.2 mmol), and N,N-dimethylformamide (41 mL). The headspace of the vial was replaced with nitrogen, the vial was sealed, and the reaction mixture was stirred in front of Kessil PR160L lights (440 nm) for 24 h. The reaction mixture was diluted with ethyl acetate (300 mL) and washed with 1M sodium hydroxide (100 mL), and saturated ammonium chloride solution (2×50 mL), dried over anhydrous magnesium sulfate, filtered, and concentrated in vacuo. Purification of the residue by column chromatography, eluting with 0 to 80% of ethyl acetate in heptane, afforded the title compound as a yellow solid (0.81 g, 79% yield): MS (ES+) m/z 410.1 (M+1).

### Step 2. Preparation of rac-2′-(5,5-difluorotetrahydro-2H-pyran-2-yl)-3-fluoro-[2,4′-bipyridin]-3′-amine

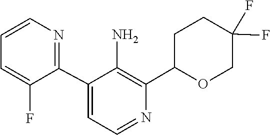 [SMILES: Nc1c(-c2ncccc2F)ccnc1C1CCC(F)(F)CO1]

To rac-tert-butyl (2′-(5,5-difluorotetrahydro-2H-pyran-2-yl)-3-fluoro-[2,4′-bipyridin]-3′-yl)carbamate (0.81 g, 2.0 mmol) was added a 4 M solution of hydrochloric acid in 1,4-dioxane (10 mL). The reaction mixture was heated to 40° C. stirred for 18 h. The reaction mixture was diluted with ethyl acetate (120 mL), washed with 1M sodium hydroxide (2×100 mL), and brine (50 mL), dried over anhydrous magnesium sulfate, filtered, and concentrated in vacuo. The residue was purified by column chromatography eluting with a gradient of 5 to 95% of ethyl acetate in heptane to provide the title compound as a slightly yellow solid (0.52 g, 86% yield): MS (ES+) m/z 310.0 (M+1).

### Step 3. Preparation of rac-N-(2′-(5,5-difluorotetrahydro-2H-pyran-2-yl)-3-fluoro-[2,4′-bipyridin]-3′-yl)-5,6-difluoronicotinamide

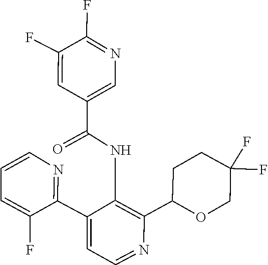 [SMILES: O=C(Nc1c(-c2ncccc2F)ccnc1C1CCC(F)(F)CO1)c1cnc(F)c(F)c1]

To a mixture of rac-2′-(5,5-difluorotetrahydro-2H-pyran-2-yl)-3-fluoro-[2,4′-bipyridin]-3′-amine (0.15 g, 0.49 mmol), 2,3-difluoropyridine-5-carboxylic acid (0.092 g, 0.58 mmol) and 2-chloro-1-methylpyridinium iodide (0.19 g, 0.73 mmol) was added anhydrous tetrahydrofuran (10 mL). The solution was stirred for 1 min before N-ethyl-N-isopropylpropan-2-amine (0.63 g, 4.8 mmol) was added. The reaction mixture was stirred at ambient temperature for 18 h. The reaction mixture was diluted with ethyl acetate (150 mL). The reaction mixture was washed with 1M sodium hydroxide (50 mL), and saturated ammonium chloride solution (2×50 mL). The organic layer was dried over anhydrous magnesium sulfate, filtered, and concentrated in vacuo. Purification of the residue by column chromatography, eluting with 10 to 75% of ethyl acetate in heptane, provided the title compound as a colorless oil (0.054 g, 25% yield): MS (ES+) m/z 450.3 (M+1).

### Step 4. Preparation of rac-N-(2′-(5,5-difluorotetrahydro-2H-pyran-2-yl)-3-fluoro-[2,4′-bipyridin]-3′-yl)-5-fluoro-6-isopropoxynicotinamide

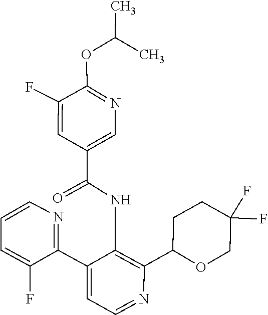 [SMILES: CC(C)Oc1ncc(C(=O)Nc2c(-c3ncccc3F)ccnc2C2CCC(F)(F)CO2)cc1F]

To a mixture of rac-N-(2′-(5,5-difluorotetrahydro-2H-pyran-2-yl)-3-fluoro-[2,4′-bipyridin]-3′-yl)-5,6-difluoronicotinamide (0.054 g, 0.12 mmol), 2-propanol (1.2 mL), and anhydrous N,N-dimethylformamide (1.2 mL) was added 60% sodium hydride dispersion in mineral oil (0.048 g, 1.2 mmol). The reaction mixture was stirred at ambient temperature for 2 h. The reaction mixture was diluted with ethyl acetate (100 mL) and washed with 1M sodium hydroxide (50 mL), and saturated ammonium chloride solution (2×50 mL). The organic layer was dried over anhydrous magnesium sulfate, filtered, and concentrated in vacuo. Purification of the residue by column chromatography, eluting with 10 to 60% of ethyl acetate in heptane provided the title compound as light pink solid (0.017 g, 29% yield): ¹H-NMR (400 MHz; DMSO-d₆) δ 10.19 (s, 1H), 8.69 (d, J=4.9 Hz, 1H), 8.45 (dt, J=3.0, 1.5 Hz, 1H), 8.37 (d, J=1.9 Hz, 1H), 7.90 (dd, J=11.1, 1.9 Hz, 1H), 7.82 (ddd, J=10.1, 8.6, 1.3 Hz, 1H), 7.60 (dd, J=4.8, 0.7 Hz, 1H), 7.50 (ddd, J=8.6, 5.5, 3.1 Hz, 1H), 5.41-5.35 (m, 1H), 4.93-4.90 (m, 1H), 3.95-3.89 (m, 1H), 3.79-3.68 (m, 1H), 2.37-2.13 (m, 3H), 2.02-1.97 (m, 1H), 1.36-1.32 (m, 6H); MS (ES+) m/z 491.2 (M+1).

### Example 314

### Synthesis of rac-2-cyclobutoxy-N-(2′-(5,5-difluorotetrahydro-2H-pyran-2-yl)-3-fluoro-[2,4′-bipyridin]-3′-yl)pyrimidine-5-carboxamide

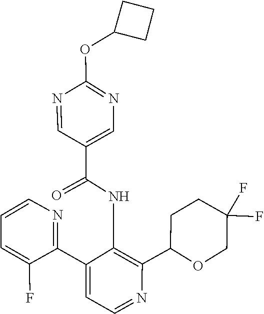 [SMILES: O=C(Nc1c(-c2ncccc2F)ccnc1C1CCC(F)(F)CO1)c1cnc(OC2CCC2)nc1]

### Step 1. Preparation of rac-2-chloro-N-(2′-(5,5-difluorotetrahydro-2H-pyran-2-yl)-3-fluoro-[2,4′-bipyridin]-3′-yl)pyrimidine-5-carboxamide

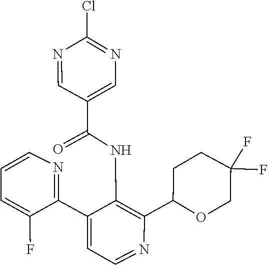 [SMILES: O=C(Nc1c(-c2ncccc2F)ccnc1C1CCC(F)(F)CO1)c1cnc(Cl)nc1]

To a mixture of rac-2′-(5,5-difluorotetrahydro-2H-pyran-2-yl)-3-fluoro-[2,4′-bipyridin]-3′-amine (0.13 g, 0.40 mmol), 2-chloropyrimidine-5-carboxylic acid (0.077 g, 0.49 mmol) and 2-chloro-1-methylpyridinium iodide (0.15 g, 0.61 mmol) was added anhydrous tetrahydrofuran (8.1 mL). The solution was stirred for 1 min before N-ethyl-N-isopropylpropan-2-amine (0.52 g, 4.0 mmol) was added. The reaction mixture was stirred at ambient temperature for 48 h. The reaction mixture was diluted with ethyl acetate (120 mL). The reaction mixture was washed with 1M sodium hydroxide (50 mL), and saturated ammonium chloride solution (2×50 mL). The organic layer was dried over anhydrous magnesium sulfate, filtered, and concentrated in vacuo. Purification of the residue by column chromatography, eluting with 10 to 60% of ethyl acetate in heptane, provided the title compound as a yellow solid (0.024 g, 13% yield): MS (ES+) m/z 450.2 (M+1), m/z 452.4 (M+1).

### Step 2. Preparation of rac-2-cyclobutoxy-N-(2′-(5,5-difluorotetrahydro-2H-pyran-2-yl)-3-fluoro-[2,4′-bipyridin]-3′-yl)pyrimidine-5-carboxamide

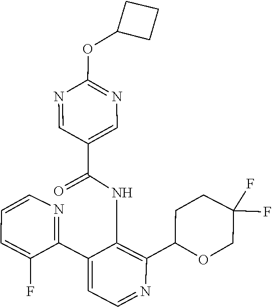 [SMILES: O=C(Nc1c(-c2ncccc2F)ccnc1C1CCC(F)(F)CO1)c1cnc(OC2CCC2)nc1]

To a mixture of rac-2-chloro-N-(2′-(5,5-difluorotetrahydro-2H-pyran-2-yl)-3-fluoro-[2,4′-bipyridin]-3′-yl)pyrimidine-5-carboxamide (0.024 g, 0.053 mmol), cyclobutanol (0.53 mL), and anhydrous N,N-dimethylformamide (0.53 mL) was added 60% sodium hydride dispersion in mineral oil (0.006 g, 0.15 mmol). The reaction mixture was stirred at ambient temperature for 6 h. The reaction mixture was diluted with ethyl acetate (100 mL) and washed with 1M sodium hydroxide (50 mL), and saturated ammonium chloride solution (2×50 mL). The organic layer was dried over anhydrous magnesium sulfate, filtered, and concentrated in vacuo. Purification of the residue by column chromatography, eluting with 10 to 60% of ethyl acetate in heptane provided the title compound as light yellow solid (0.014 g, 51% yield): ¹H-NMR (400 MHz; DMSO-d₆) δ 10.30 (s, 1H), 8.86-8.82 (m, 2H), 8.70 (d, J=4.9 Hz, 1H), 8.46 (dt, J=3.1, 1.5 Hz, 1H), 7.83 (ddd, J=10.1, 8.6, 1.3 Hz, 1H), 7.60 (dd, J=4.8, 1.0 Hz, 1H), 7.52-7.48 (m, 1H), 5.22-5.14 (m, 1H), 4.94-4.92 (m, 1H), 3.95-3.88 (m, 1H), 3.83-3.72 (m, 1H), 2.46-2.37 (m, 2H), 2.35-2.24 (m, 2H), 2.23-2.05 (m, 3H), 2.02-1.96 (m, 1H), 1.85-1.76 (m, 1H), 1.71-1.62 (m, 1H); MS (ES+) m/z 486.2 (M+1).

### Example 315

### Synthesis of N-(2-(4,4-difluorocyclohexyl)-4-(2,5-difluorophenyl)pyridin-3-yl)-2-isopropyl-N-methylpyrimidine-5-carboxamide

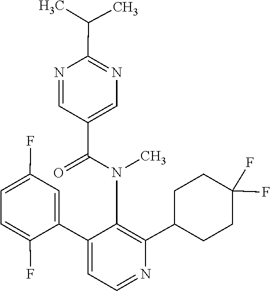 [SMILES: CC(C)c1ncc(C(=O)N(C)c2c(-c3cc(F)ccc3F)ccnc2C2CCC(F)(F)CC2)cn1]

### Step 1. Preparation of 2-(4,4-difluorocyclohexyl)-4-(2,5-difluorophenyl)-N-methylpyridin-3-amine hydrogen chloride salt

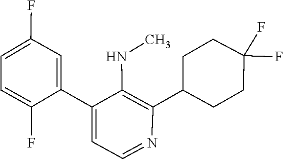 [SMILES: CNc1c(-c2cc(F)ccc2F)ccnc1C1CCC(F)(F)CC1]

To a mixture of tert-butyl (2-(4,4-difluorocyclohexyl)-4-(2,5-difluorophenyl)pyridin-3-yl)carbamate (0.15 g, 0.35 mmol), and anhydrous N,N-dimethylformamide (3.5 mL) was added 60% sodium hydride dispersion in mineral oil (0.056 g, 1.4 mmol). The reaction was stirred at ambient temperature for 20 min. Iodomethane (0.15 g, 1.1 mmol) was added to the reaction mixture at ambient temperature and the reaction mixture was stirred for 30 min. The reaction mixture was diluted with ethyl acetate (100 mL) and washed with saturated ammonium chloride solution (3×50 mL). The organic layer was dried over anhydrous magnesium sulfate, filtered, and concentrated in vacuo. The reside was dissolved in 4M hydrogen chloride in 1,4-dioxane (9 mL) and stirred at 45° C. for 3 h. The reaction mixture was concentrated in vacuo and purification of the residue by column chromatography, eluting with 10 to 100% of ethyl acetate in heptane followed by 0 to 16% of methanol in ethyl acetate, provided the title compound as a colorless oil (0.13 g, 99% yield): MS (ES+) m/z 339.4 (M+1).

### Step 2. Preparation of N-(2-(4,4-difluorocyclohexyl)-4-(2,5-difluorophenyl)pyridin-3-yl)-2-isopropyl-N-methylpyrimidine-5-carboxamide

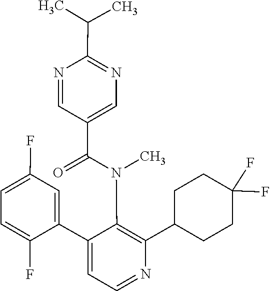 [SMILES: CC(C)c1ncc(C(=O)N(C)c2c(-c3cc(F)ccc3F)ccnc2C2CCC(F)(F)CC2)cn1]

To a mixture of 2-(4,4-difluorocyclohexyl)-4-(2,5-difluorophenyl)-N-methylpyridin-3-amine hydrogen chloride salt (0.13 g, 0.35 mmol), 2-isopropylpyrimidine-5-carboxylic acid (0.16 g, 0.94 mmol) and 2-chloro-1-methylpyridinium iodide (0.54 g, 2.1 mmol) was added anhydrous tetrahydrofuran (9.4 mL). The solution was heated to 50° C. for 1 min before N-ethyl-N-isopropylpropan-2-amine (0.91 g, 7.0 mmol) was added. The reaction mixture was stirred at 50° C. for 18 h. The reaction mixture was diluted with ethyl acetate (100 mL). The reaction mixture was washed with 1M sodium hydroxide (50 mL), and saturated ammonium chloride solution (2×50 mL). The organic layer was dried over anhydrous magnesium sulfate, filtered, and concentrated in vacuo. Purification of the residue by column chromatography, eluting with 10 to 60% of ethyl acetate in heptane, followed by reverse phase column chromatography, eluting with a gradient of 10 to 95% of acetonitrile in water, provided the title compound as a colorless solid (0.024 g, 13% yield): ¹H-NMR (400 MHz; DMSO-d₆, 2:1 mixture of rotamers) δ 8.69-8.68 (m, 1H), 8.67 (dd, J=5.1, 2.9 Hz, 2H), 8.63 (d, J=2.8 Hz, 2H), 7.97-7.95 (m, 4H), 7.53-7.44 (m, 2H), 7.43-7.36 (m, 3H), 7.30-7.24 (m, 2H), 7.23 (dd, J=4.9, 1.2 Hz, 2H), 7.22-7.17 (m, 1H), 6.81-6.77 (m, 2H), 3.37 (s, 6H), 3.34-3.28 (m, 2H), 3.19 (dt, J=13.8, 6.9 Hz, 1H), 3.10-3.04 (m, 2H), 3.03 (d, J=3.1 Hz, 3H), 2.21-1.81 (m, 23H), 1.65-1.61 (m, 2H), 1.29 (t, J=6.0 Hz, 6H), 1.19 (dd, J=6.9, 2.8 Hz, 12H); MS (ES+) m/z 487.2 (M+1).

### Example 316

### Synthesis of 6-cyano-N-(2-(4,4-difluorocyclohexyl)-4-(2,5-difluorophenyl)pyridin-3-yl)-5-fluoronicotinamide

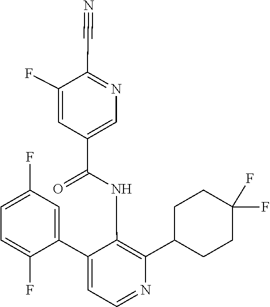 [SMILES: N#Cc1ncc(C(=O)Nc2c(-c3cc(F)ccc3F)ccnc2C2CCC(F)(F)CC2)cc1F]

### Step 1. Preparation of 6-bromo-N-(2-(4,4-difluorocyclohexyl)-4-(2,5-difluorophenyl)pyridin-3-yl)-5-fluoronicotinamide

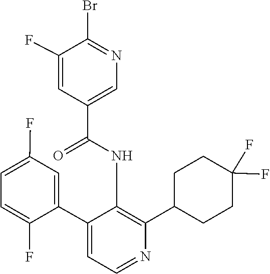 [SMILES: O=C(Nc1c(-c2cc(F)ccc2F)ccnc1C1CCC(F)(F)CC1)c1cnc(Br)c(F)c1]

To a mixture of 2-(4,4-difluorocyclohexyl)-4-(2,5-difluorophenyl)pyridin-3-amine (0.22 g, 0.67 mmol), 6-bromo-5-fluoro-pyridine-3-carboxylic acid (0.22 g, 1.0 mmol) and 2-chloro-1-methylpyridinium iodide (0.51 g, 2.0 mmol) was added anhydrous tetrahydrofuran (13 mL). The solution was heated at 50° C. for 1 min before N-ethyl-N-isopropylpropan-2-amine (1.3 g, 10 mmol) was added. The reaction mixture was stirred at 50° C. for 8 h. The reaction mixture was diluted with methanol (5 mL), and 5M sodium hydroxide (1 mL) and heated to 50° C. for 2 h. The reaction mixture was diluted with ethyl acetate (150 mL). The reaction mixture was washed with 1M sodium hydroxide (50 mL), saturated ammonium chloride solution (2×50 mL). The organic layer was dried over anhydrous magnesium sulfate, filtered, and concentrated in vacuo. Purification of the residue by column chromatography, eluting with 10 to 60% of ethyl acetate in heptane, provided the title compound as a colorless solid (0.30 g, 85% yield): ¹H-NMR (400 MHz; CDCl₃) δ 8.73-8.71 (m, 1H), 8.48-8.47 (m, 1H), 7.83-7.80 (m, 1H), 7.61-7.59 (m, 1H), 7.23 (t, J=3.9 Hz, 1H), 7.18-7.06 (m, 3H), 3.02-2.94 (m, 1H), 2.31-2.12 (m, 4H), 2.00-1.92 (m, 2H), 1.88-1.71 (m, 2H); MS (ES+) m/z 527.3 (M+1), m/z 529.4 (M+1).

### Step 2. Preparation of 6-cyano-N-(2-(4,4-difluorocyclohexyl)-4-(2,5-difluorophenyl)pyridin-3-yl)-5-fluoronicotinamide

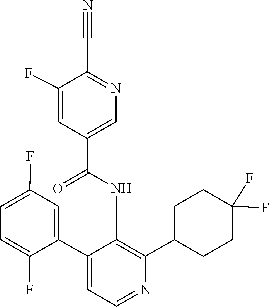 [SMILES: N#Cc1ncc(C(=O)Nc2c(-c3cc(F)ccc3F)ccnc2C2CCC(F)(F)CC2)cc1F]

A mixture of 6-bromo-N-(2-(4,4-difluorocyclohexyl)-4-(2,5-difluorophenyl)pyridin-3-yl)-5-fluoronicotinamide (0.30 g, 0.57 mmol), zinc (II) cyanide (0.049 g, 0.42 mmol), tetrakis(triphenylphosphine)palladium(0) (0.065 g, 0.057 mmol) and anhydrous N,N-dimethylformamide (1.1 mL) was sparged with nitrogen for 5 min.

The microwave vial was sealed and heated to 150° C. for 15 min under microwave irradiation. The reaction mixture was cooled to ambient temperature and diluted with ethyl acetate (125 mL). The organic phase was washed with 1M sodium hydroxide (50 mL), water (50 mL), and brine (50 mL). The organic layer was dried over anhydrous magnesium sulfate, filtered, and concentrated in vacuo. Purification of the residue by column chromatography, eluting with 10 to 60% of ethyl acetate in heptane, afforded the title compound as a colorless solid (0.23 g, 86% yield): ¹H-NMR (400 MHz; DMSO-d₆) δ 10.62-10.60 (m, 1H), 8.82 (d, J=1.3 Hz, 1H), 8.65 (d, J=4.9 Hz, 1H), 8.33-8.30 (m, 1H), 7.40 (d, J=4.9 Hz, 1H), 7.39-7.23 (m, 3H), 3.22-3.15 (m, 1H), 2.13-2.05 (m, 2H), 2.05-1.78 (m, 6H); MS (ES+) m/z 473.2 (M+1).

### Example 317

### Synthesis of N-5-(2-(4,4-difluorocyclohexyl)-4-(2,5-difluorophenyl)pyridin-3-yl)-3-fluoro-N²-isopropylpyridine-2,5-dicarboxamide

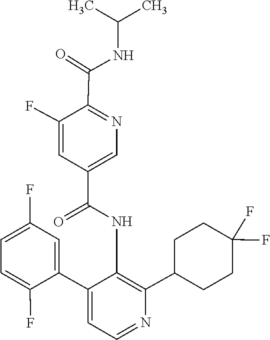 [SMILES: CC(C)NC(=O)c1ncc(C(=O)Nc2c(-c3cc(F)ccc3F)ccnc2C2CCC(F)(F)CC2)cc1F]

### Step 1. Preparation of 5-((2-(4,4-difluorocyclohexyl)-4-(2,5-difluorophenyl)pyridin-3-yl)carbamoyl)-3-fluoropicolinic acid

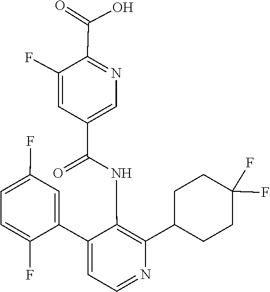 [SMILES: O=C(Nc1c(-c2cc(F)ccc2F)ccnc1C1CCC(F)(F)CC1)c1cnc(C(=O)O)c(F)c1]

To a mixture of 6-cyano-N-(2-(4,4-difluorocyclohexyl)-4-(2,5-difluorophenyl)pyridin-3-yl)-5-fluoronicotinamide (0.22 g, 0.47 mmol) and tetrahydrofuran (1 mL), was added concentrated hydrochloric acid (8.0 mL). The solution was heated at 70° C. for 18 h. The reaction mixture was concentrated in vacuo. The residue was purified by reverse phase column chromatography eluting with a gradient of 10 to 95% of acetonitrile in water, to furnish the title compound as an impure mixture used in the next step as is (0.11 g, 28% yield): MS (ES+) m/z 492.4 (M+1).

### Step 2. Preparation of N5-(2-(4,4-difluorocyclohexyl)-4-(2,5-difluorophenyl)pyridin-3-yl)-3-fluoro-N²-isopropylpyridine-2,5-dicarboxamide

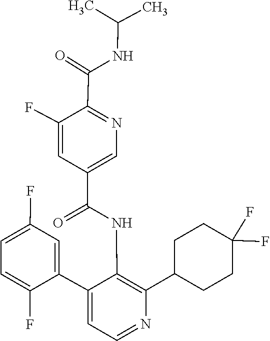 [SMILES: CC(C)NC(=O)c1ncc(C(=O)Nc2c(-c3cc(F)ccc3F)ccnc2C2CCC(F)(F)CC2)cc1F]

A mixture of 5-((2-(4,4-difluorocyclohexyl)-4-(2,5-difluorophenyl)pyridin-3-yl)carbamoyl)-3-fluoropicolinic acid (0.11 g, 0.22 mmol), and anhydrous dichloromethane (2.2 mL) was cooled in an ice-water bath before oxalyl chloride (0.055 g, 0.43 mmol) and anhydrous N,N-dimethylformamide (0.01 mL) was added. The cooled reaction mixture was stirred for 1 h before a mixture of isopropylamine (0.13 g, 2.2 mmol), N-ethyl-N-isopropylpropan-2-amine (0.28 g, 0.38 mmol), and anhydrous dichloromethane (2.2 mL) was added. The reaction mixture was warmed to ambient temperature and stirred for 3 h. The reaction mixture was diluted with ethyl acetate (100 mL) and washed with saturated ammonium chloride (2×50 mL). The organic layer was dried over anhydrous magnesium sulfate, filtered, and concentrated in vacuo. Purification of the residue by column chromatography, eluting with 20 to 90% of ethyl acetate in heptane, afforded the title compound as a colorless solid (0.23 g, 86% yield): ¹H-NMR (400 MHz; DMSO-d₆) δ 1H-NMR (400 MHz; DMSO-d₆): 510.45 (s, 1H), 8.67-8.66 (m, 1H), 8.65 (dd, J=5.2, 2.8 Hz, 1H), 8.60-8.58 (m, 1H), 8.10 (dd, J=10.5, 1.6 Hz, 1H), 7.40 (d, J=4.9 Hz, 1H), 7.38-7.23 (m, 3H), 4.12-4.04 (m, 1H), 3.23-3.16 (m, 1H), 2.15-2.05 (m, 2H), 2.03-1.80 (m, 6H), 1.18-1.14 (m, 6H); MS (ES+) m/z 533.2 (M+1).

### Example 318

### Synthesis of rac-6-(azetidin-2-yl)-N-(2-(4,4-difluorocyclohexyl)-4-(2,5-difluorophenyl)pyridin-3-yl)-5-fluoronicotinamide

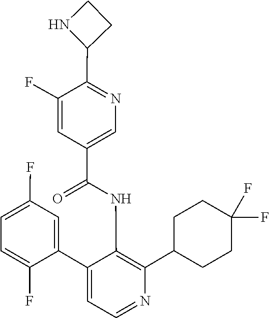 [SMILES: O=C(Nc1c(-c2cc(F)ccc2F)ccnc1C1CCC(F)(F)CC1)c1cnc(C2CCN2)c(F)c1]

### Step 1. Preparation of 6-chloro-N-(2-(4,4-difluorocyclohexyl)-4-(2,5-difluorophenyl)pyridin-3-yl)-5-fluoronicotinamide

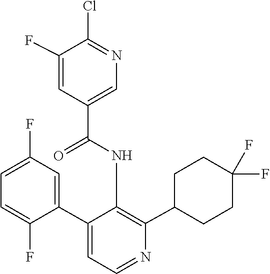 [SMILES: O=C(Nc1c(-c2cc(F)ccc2F)ccnc1C1CCC(F)(F)CC1)c1cnc(Cl)c(F)c1]

To a mixture of 2-(4,4-difluorocyclohexyl)-4-(2,5-difluorophenyl)pyridin-3-amine (0.090 g, 0.28 mmol), 6-chloro-5-fluoronicotinic acid (0.073 g, 0.42 mmol) and 2-chloro-1-methylpyridinium iodide (0.22 g, 0.83 mmol) was added anhydrous tetrahydrofuran (5.6 mL). The solution was heated at 50° C. for 1 min before N-ethyl-N-isopropylpropan-2-amine (0.54 g, 4.2 mmol) was added. The reaction mixture was stirred at 50° C. for 3 h. The reaction mixture was cooled to ambient temperature and diluted with methanol (5 mL), 5M sodium hydroxide solution (3 mL) and stirred for 1 h. The reaction mixture was further diluted with ethyl acetate (100 mL) and washed with 1M sodium hydroxide (50 mL), and saturated ammonium chloride solution (2×50 mL). The organic layer was dried over anhydrous magnesium sulfate, filtered, and concentrated in vacuo. Purification of the residue by column chromatography, eluting with 10 to 60% of ethyl acetate in heptane, provided the title compounds as a colorless solid (0.13 g, 100% yield): ¹H-NMR (400 MHz; CDCl₃) δ 8.75-8.70 (m, 1H), 8.52-8.47 (m, 1H), 7.91-7.86 (m, 1H), 7.63-7.56 (m, 1H), 7.26-7.21 (m, 1H), 7.19-7.06 (m, 3H), 3.03-2.94 (m, 1H), 2.32-2.11 (m, 4H), 2.00-1.91 (m, 2H), 1.89-1.70 (m, 2H); MS (ES+) m/z 482.4 (M+1); 484.4 (M+1).

### Step 2. Preparation of rac-6-(azetidin-2-yl)-N-(2-(4,4-difluorocyclohexyl)-4-(2,5-difluorophenyl)pyridin-3-yl)-5-fluoronicotinamide

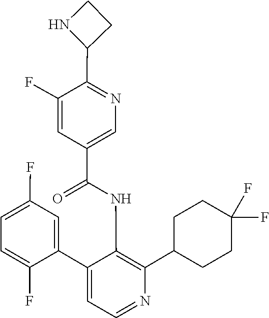 [SMILES: O=C(Nc1c(-c2cc(F)ccc2F)ccnc1C1CCC(F)(F)CC1)c1cnc(C2CCN2)c(F)c1]

To 6-chloro-N-(2-(4,4-difluorocyclohexyl)-4-(2,5-difluorophenyl)pyridin-3-yl)-5-fluoronicotinamide (0.13 g, 0.28 mmol) was added azetidine-1,2-dicarboxylic acid 1-tert-butyl ester (0.088 g, 0.44 mmol), (4,4″-di-t-butyl-2,2″-bipyridine)bis[3,5-difluoro-2-[5-trifluoromethyl-2-pyridinyl-kN)phenyl-kC]iridium(III) hexafluorophosphate (0.0033 g, 0.0029 mmol), dichloro(dimethoxyethane)nickel (0.0064 g, 0.029 mmol), 4-tert-butyl-2-(4-tert-butyl-2-pyridyl)pyridine (0.012 g, 0.044 mmol), cesium carbonate (0.16 g, 0.50 mmol), and N,N-dimethylformamide (7.3 mL). The headspace of the vial was replaced with nitrogen, the vial was sealed, and the reaction mixture was stirred in front of Kessil PR160L lights (440 nm) for 24 h. The reaction mixture was diluted with ethyl acetate (50 mL) and washed with 1M sodium hydroxide (25 mL), saturated ammonium chloride solution (2×25 mL), dried over anhydrous magnesium sulfate, filtered, and concentrated in vacuo. Purification of the residue was achieved by column chromatography, eluting with a gradient of 10 to 70% of ethyl acetate in heptane. The colorless oil was dissolved in trifluoroacetic acid (2.2 mL) and stirred for 2 h at ambient temperature. The reaction mixture was diluted with ethyl acetate (100 mL) and washed with a 1:1 mixture of 1M sodium hydroxide:brine (2×50 mL), dried over anhydrous magnesium sulfate, filtered, and concentrated in vacuo. Purification of the residue by column chromatography, eluting with 20 to 100% ethyl acetate in heptane followed by 0 to 25% methanol in ethyl acetate, afforded the title compound as a colorless solid (0.056 g, 38% yield): MS (ES+) m/z: 503.2 (M+1).

### Example 319

### Synthesis of rac-N-(2-(4,4-difluorocyclohexyl)-4-(2,5-difluorophenyl)pyridin-3-yl)-5-fluoro-6-(pyrrolidin-2-yl)nicotinamide trifluoroacetic acid salt

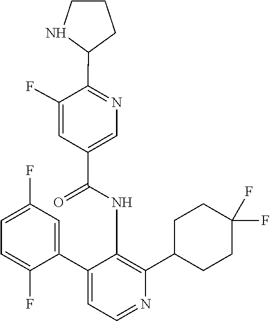 [SMILES: O=C(Nc1c(-c2cc(F)ccc2F)ccnc1C1CCC(F)(F)CC1)c1cnc(C2CCCN2)c(F)c1]

To rac-6-chloro-N-(2-(4,4-difluorocyclohexyl)-4-(2,5-difluorophenyl)pyridin-3-yl)-5-fluoronicotinamide (0.10 g, 0.21 mmol) was added (tert-butoxycarbonyl)proline (0.067 g, 0.31 mmol), (4,4″-di-t-butyl-2,2″-bipyridine)bis[3,5-difluoro-2-[5-trifluoromethyl-2-pyridinyl-kN)phenyl-kC]iridium(II) hexafluorophosphate (0.0023 g, 0.0021 mmol), dichloro(dimethoxyethane)nickel (0.0046 g, 0.021 mmol), 4-tert-butyl-2-(4-tert-butyl-2-pyridyl)pyridine (0.0084 g, 0.031 mmol), cesium carbonate (0.11 g, 0.35 mmol), and N,N-dimethylformamide (5.2 mL). The headspace of the vial was replaced with nitrogen, the vial was sealed, and the reaction mixture was stirred in front of Kessil PR160L lights (440 nm) for 24 h. The reaction mixture was diluted with ethyl acetate (100 mL) and washed with 1M sodium hydroxide (50 mL), saturated ammonium chloride solution (50 mL), dried over anhydrous magnesium sulfate, filtered, and concentrated in vacuo. Purification of the residue by column chromatography, eluting with a gradient of 10 to 70% of ethyl acetate in heptane to a afford an impure mixture which was immediately dissolved in anhydrous dichloromethane (3.0 mL), and trifluoroacetic acid (3.0 mL) and stirred for 1 h. The reaction mixture was concentrated in vacuo. Purification of the residue by column chromatography, eluting with a gradient of 50 to 100% of ethyl acetate in heptane, followed by 0 to 25% of methanol in ethyl acetate, afforded the title compound as a colorless solid (0.031 g, 28% yield): ¹H-NMR (400 MHz; DMSO-d₆) δ 10.53-10.50 (m, 1H), 9.42-9.25 (m, 2H), 8.74-8.72 (m, 1H), 8.67-8.64 (m, 1H), 8.16-8.12 (m, 1H), 7.40-7.38 (m, 1H), 7.37-7.24 (m, 3H), 5.07-5.03 (m, 1H), 3.44-3.37 (m, 1H), 3.22-3.18 (m, 1H), 2.49-2.42 (m, 1H), 2.17-1.78 (m, 12H); MS (ES+) m/z 517.2 (M+1).

### Example 320

### Synthesis of N-(4-(4,4-difluorocyclohexyl)-2-(2,5-difluorophenyl)pyridin-3-yl)-2-isopropylpyrimidine-5-carboxamide

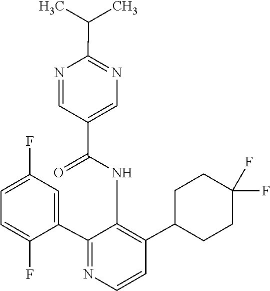 [SMILES: CC(C)c1ncc(C(=O)Nc2c(C3CCC(F)(F)CC3)ccnc2-c2cc(F)ccc2F)cn1]

To a mixture of 4-(4,4-difluorocyclohexyl)-2-(2,5-difluorophenyl)pyridin-3-amine (0.16 g, 0.44 mmol), 2-isopropylpyrimidine-5-carboxylic acid (0.13 g, 0.75 mmol) and 2-chloro-1-methylpyridinium iodide (0.34 g, 1.3 mmol) was added anhydrous tetrahydrofuran (8.9 mL). The solution was heated at 60° C. for 1 min before N-ethyl-N-isopropylpropan-2-amine (0.57 g, 4.4 mmol) was added. The reaction mixture was stirred at 50° C. for 3 h. The reaction mixture was cooled to ambient temperature and diluted with methanol (5 mL), 5M sodium hydroxide solution (3 mL) and stirred for 1 h at 50° C. The reaction mixture was further diluted with ethyl acetate (100 mL) and washed with 1M sodium hydroxide (50 mL), and saturated ammonium chloride solution (2×50 mL). The organic layer was dried over anhydrous magnesium sulfate, filtered, and concentrated in vacuo. Purification of the residue by column chromatography, eluting with a gradient of 10 to 60% of ethyl acetate in heptane, provided the title compound as a colorless solid (0.13 g, 60% yield): ¹H-NMR (400 MHz; DMSO-d₆) S 10.37 (s, 1H), 8.95 (s, 2H), 8.61 (d, J=5.1 Hz, 1H), 7.56 (d, J=5.2 Hz, 1H), 7.35-7.21 (m, 3H), 3.20 (dt, J=13.8, 6.9 Hz, 1H), 3.07-3.00 (m, 1H), 2.11-1.83 (m, 6H), 1.77-1.67 (m, 2H), 1.29 (d, J=6.9 Hz, 6H); MS (ES+) m/z 473.2 (M+1).

### Example 321

### Synthesis of rac-N-(4-(2,5-difluorophenyl)-2-((anti)-2-methyl-4-oxocyclohexyl)pyridin-3-yl)-2-isopropylpyrimidine-5-carboxamide

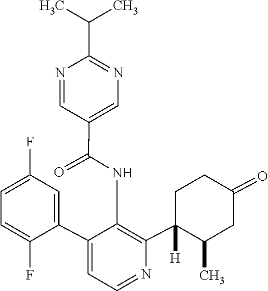 [SMILES: CC(C)c1ncc(C(=O)Nc2c(-c3cc(F)ccc3F)ccnc2[C@@H]2CCC(=O)C[C@H]2C)cn1]

### Step 1. Preparation of rac-tert-butyl (4-(2,5-difluorophenyl)-2-((anti)-2-methyl-4-oxocyclohexyl)pyridin-3-yl)carbamate

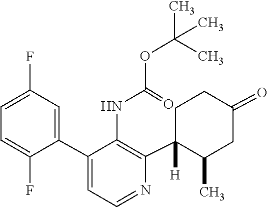 [SMILES: C[C@@H]1CC(=O)CC[C@H]1c1nccc(-c2cc(F)ccc2F)c1NC(=O)OC(C)(C)C]

To tert-butyl (2-chloro-4-(2,5-difluorophenyl)pyridin-3-yl)carbamate (0.50 g, 1.5 mmol) was added 2-methyl-4-oxo-cyclohexanecarboxylic acid (0.46 g, 2.9 mmol), (4,4″-di-t-butyl-2,2″-bipyridine)bis[3,5-difluoro-2-[5-trifluoromethyl-2-pyridinyl-kN)phenyl-kC]iridium(II) hexafluorophosphate (0.016 g, 0.015 mmol), dichloro(dimethoxyethane)nickel (0.032 g, 0.15 mmol), 4-tert-butyl-2-(4-tert-butyl-2-pyridyl)pyridine (0.059 g, 0.22 mmol), cesium carbonate (1.0 g, 3.1 mmol), and N,N-dimethylacetamide (37 mL). The headspace of the vial was replaced with nitrogen, the vial was sealed, and the reaction mixture was stirred in front of Kessil PR160L lights (440 nm) for 24 h. The reaction mixture was diluted with ethyl acetate (250 mL) and washed with 1M sodium hydroxide (50 mL), water (2×50 mL), saturated ammonium chloride solution (100 mL), dried over anhydrous magnesium sulfate, filtered, and concentrated in vacuo. Purification of the residue by column chromatography, eluting with 5 to 75% of ethyl acetate in heptane, afforded the title compound as a colorless oil (0.61 g, 100% yield): MS (ES+) m/z 417.4 (M+1).

### Step 2. Preparation of rac-(anti)-4-(3-amino-4-(2,5-difluorophenyl)pyridin-2-yl)-3-methylcyclohexan-1-one

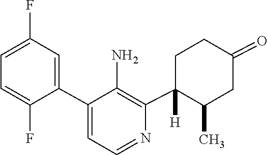 [SMILES: C[C@@H]1CC(=O)CC[C@H]1c1nccc(-c2cc(F)ccc2F)c1N]

To rac-tert-butyl (4-(2,5-difluorophenyl)-2-((anti)-2-methyl-4-oxocyclohexyl)pyridin-3-yl)carbamate (0.61 g, 1.5 mmol) was added 4M hydrogen chloride in 1,4-dioxane (7.8 mL). The reaction mixture was stirred at ambient temperature for 3 h. The reaction mixture was diluted with ethyl acetate (200 mL) and washed with 1M sodium hydroxide (2×50 mL), saturated ammonium chloride solution (50 mL), dried over anhydrous magnesium sulfate, filtered, and concentrated in vacuo. Purification of the residue by column chromatography, eluting with 15 to 100% of ethyl acetate in heptane, afforded the title compound as a colorless solid (0.25 g, 51% yield): MS (ES+) m/z 317.4 (M+1).

### Step 3. Preparation of rac-N-(4-(2,5-difluorophenyl)-2-((anti)-2-methyl-4-oxocyclohexyl)pyridin-3-yl)-2-isopropylpyrimidine-5-carboxamide

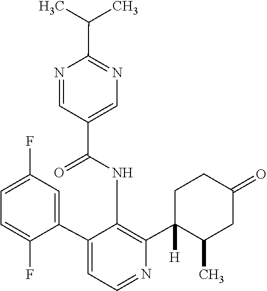 [SMILES: CC(C)c1ncc(C(=O)Nc2c(-c3cc(F)ccc3F)ccnc2[C@@H]2CCC(=O)C[C@H]2C)cn1]

To a mixture of rac-(anti)-4-(3-amino-4-(2,5-difluorophenyl)pyridin-2-yl)-3-methylcyclohexan-1-one (0.063 g, 0.20 mmol), 2-isopropylpyrimidine-5-carboxylic acid (0.068 g, 0.40 mmol) and 2-chloro-1-methylpyridinium iodide (0.14 g, 0.56 mmol) was added anhydrous tetrahydrofuran (8.0 mL). The solution was heated at 60° C. for 1 min before N-ethyl-N-isopropylpropan-2-amine (0.51 g, 4.0 mmol) was added. The reaction mixture was stirred at 50° C. for 3 h. The reaction mixture was cooled to ambient temperature and diluted with methanol (5 mL), 5M sodium hydroxide solution (3 mL) and stirred for 1 h. The reaction mixture was further diluted with ethyl acetate (150 mL) and washed with 1M sodium hydroxide (50 mL), and saturated ammonium chloride solution (50 mL). The organic layer was dried over anhydrous magnesium sulfate, filtered, and concentrated in vacuo. Purification of the residue by column chromatography, eluting with a gradient of 10 to 100% of ethyl acetate in heptane, followed by reverse phase column chromatography, eluting with a gradient of 10 to 95% of acetonitrile in water containing 0.5% formic acid, provided the title compound as a colorless solid (0.053 g, 56% yield): ¹H-NMR (400 MHz; DMSO-d₆) δ 10.47-10.42 (m, 1H), 8.99 (s, 2H), 8.66 (d, J=4.9 Hz, 1H), 7.41-7.35 (m, 2H), 7.29-7.26 (m, 2H), 3.24-3.17 (m, 2H), 2.55-2.47 (m, 1H), 2.36-2.22 (m, 4H), 2.15-2.10 (m, 1H), 1.90-1.85 (m, 1H), 1.30-1.25 (m, 6H), 0.69-0.65 (m, 3H); MS (ES+) m/z 465.4 (M+1).

### Example 322

### Synthesis of rac-N-(4-(2,5-difluorophenyl)-2-((anti)-2-methyl-4-oxocyclohexyl)pyridin-3-yl)-2-isopropylpyrimidine-5-carboxamide

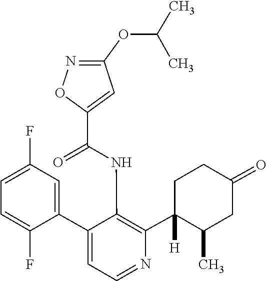 [SMILES: CC(C)Oc1cc(C(=O)Nc2c(-c3cc(F)ccc3F)ccnc2[C@@H]2CCC(=O)C[C@H]2C)on1]

To a mixture of rac-(anti)-4-(3-amino-4-(2,5-difluorophenyl)pyridin-2-yl)-3-methylcyclohexan-1-one (0.063 g, 0.20 mmol), 3-isopropoxyisoxazole-5-carboxylic acid (0.068 g, 0.40 mmol) and 2-chloro-1-methylpyridinium iodide (0.14 g, 0.56 mmol) was added anhydrous tetrahydrofuran (8.0 mL). The solution was heated at 60° C. for 1 min before N-ethyl-N-isopropylpropan-2-amine (0.51 g, 4.0 mmol) was added. The reaction mixture was stirred at 50° C. for 3 h. The reaction mixture was cooled to ambient temperature and diluted with methanol (5 mL), 5M sodium hydroxide solution (3 mL) and stirred for 1 h. The reaction mixture was further diluted with ethyl acetate (150 mL) and washed with 1M sodium hydroxide (50 mL), and saturated ammonium chloride solution (50 mL). The organic layer was dried over anhydrous magnesium sulfate, filtered, and concentrated in vacuo. Purification of the residue by column chromatography, eluting with 10 to 100% of ethyl acetate in heptane followed by reverse phase column chromatography, eluting with a gradient of 10 to 95% of acetonitrile in water containing 0.5% formic acid, provided the title compound as a colorless solid (0.068 g, 72% yield): ¹H-NMR (400 MHz; DMSO-d₆) δ 10.70 (d, J=0.2 Hz, 1H), 8.65 (d, J=4.9 Hz, 1H), 7.39-7.36 (m, 2H), 7.34-7.24 (m, 2H), 6.82 (s, 1H), 4.83 (dt, J=12.2, 6.1 Hz, 1H), 3.15-3.10 (m, 1H), 2.55-2.46 (m, 1H), 2.37-2.23 (m, 4H), 2.13-2.08 (m, 1H), 1.87-1.83 (m, 1H), 1.33 (t, J=7.8 Hz, 6H), 0.67-0.63 (m, 3H); MS (ES+) m/z 470.2 (M+1).

### Example 323

### Synthesis of N-(2-(4,4-difluorocyclohexyl)-4-(2,5-difluorophenyl)pyridin-3-yl)-5-fluoro-6-(2-hydroxypropan-2-yl)nicotinamide

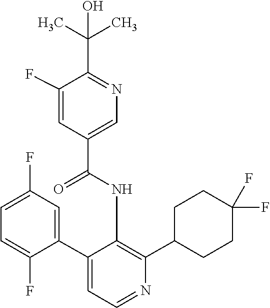 [SMILES: CC(C)(O)c1ncc(C(=O)Nc2c(-c3cc(F)ccc3F)ccnc2C2CCC(F)(F)CC2)cc1F]

### Step 1. Preparation of N-(2-(4,4-difluorocyclohexyl)-4-(2,5-difluorophenyl)pyridin-3-yl)-6-(1-ethoxyvinyl)-5-fluoronicotinamide

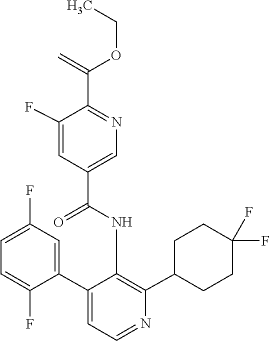 [SMILES: C=C(OCC)c1ncc(C(=O)Nc2c(-c3cc(F)ccc3F)ccnc2C2CCC(F)(F)CC2)cc1F]

A mixture of 6-chloro-N-(2-(4,4-difluorocyclohexyl)-4-(2,5-difluorophenyl)pyridin-3-yl)-5-fluoronicotinamide (0.75 g, 1.6 mmol) and N,N-dimethylformamide (20 mL) was sparged with nitrogen for 10 min. The reaction vessel was sealed following addition of tributyl(1-ethoxyvinyl)stannane (0.84 g, 2.3 mmol) and [1,1′-bis(diphenylphosphino)ferrocene]dichloropalladium(II) dichloromethane complex (0.26 g, 0.31 mmol) and heated to 100° C. for 1 h. The reaction was cooled to ambient temperature the reaction mixture was diluted with ethyl acetate (300 mL) and washed with 1M sodium hydroxide (3×50 mL), water (2×50 mL), and brine (50 mL). The organic fraction was dried over anhydrous magnesium sulfate, filtered, and concentrated in vacuo. Purification of the residue by column chromatography, eluting with a gradient of 15 to 75% of ethyl acetate in heptane, afforded the title compound as a colorless oil (0.41 g, 51% yield): MS (ES+) m/z 518.4 (M+1).

### Step 2. Preparation of 6-acetyl-N-(2-(4,4-difluorocyclohexyl)-4-(2,5-difluorophenyl)pyridin-3-yl)-5-fluoronicotinamide

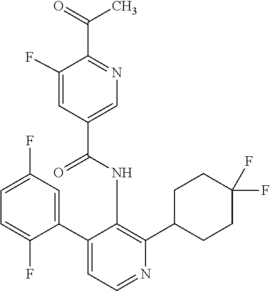 [SMILES: CC(=O)c1ncc(C(=O)Nc2c(-c3cc(F)ccc3F)ccnc2C2CCC(F)(F)CC2)cc1F]

To a mixture of 6-acetyl-N-(2-(4,4-difluorocyclohexyl)-4-(2,5-difluorophenyl)pyridin-3-yl)-5-fluoronicotinamide (0.41 g, 0.79 mmol), and anhydrous tetrahydrofuran (3.9 mL) in an ice/water bath was added 4M hydrochloric acid in water (2.8 mL). The reaction mixture was allowed to warm to ambient temperature and stirred for 2 h. The reaction mixture was diluted with ethyl acetate (300 mL) and washed with 1M sodium hydroxide (10 mL), and saturated ammonium chloride solution (50 mL). The organic layer was dried over anhydrous magnesium sulfate, filtered, and concentrated in vacuo. The residue was used as is without further purification (0.38 g, 99% yield): MS (ES+) m/z 490.4 (M+1).

### Step 3. Preparation of N-(2-(4,4-difluorocyclohexyl)-4-(2,5-difluorophenyl)pyridin-3-yl)-5-fluoro-6-(2-hydroxypropan-2-yl)nicotinamide

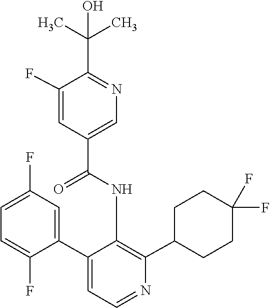 [SMILES: CC(C)(O)c1ncc(C(=O)Nc2c(-c3cc(F)ccc3F)ccnc2C2CCC(F)(F)CC2)cc1F]

To a mixture of 6-acetyl-N-(2-(4,4-difluorocyclohexyl)-4-(2,5-difluorophenyl)pyridin-3-yl)-5-fluoronicotinamide (0.41 g, 0.79 mmol), and anhydrous tetrahydrofuran (8.2 mL) in an ice/water bath was added 3M methylmagnesium bromide in tetrahydrofuran (2.2 mL). The reaction mixture was kept in the ice/water bath for 2 h. The reaction mixture was diluted with ethyl acetate (150 mL) and washed with saturated ammonium chloride solution (2×50 mL). The organic layer was dried over anhydrous magnesium sulfate, filtered, and concentrated in vacuo. Purification of the residue by column chromatography, eluting with a gradient of 15 to 75% of ethyl acetate in heptane, followed by reverse phase column chromatography, eluting with a gradient of 20 to 95% of acetonitrile in water, provided the title compound as a colorless solid (0.065 g, 15% yield): ¹H-NMR (400 MHz; CD₃CN) δ 8.70 (s, 1H), 8.66 (dd, J=4.2, 2.5 Hz, 2H), 7.83 (dd, J=11.4, 1.7 Hz, 1H), 7.33 (dd, J=4.9, 0.7 Hz, 1H), 7.24-7.13 (m, 3H), 4.94 (s, 1H), 3.20-3.18 (m, 1H), 2.19-2.12 (m, 3H), 2.04-1.94 (m, 2H), 1.90-1.86 (m, 3H), 1.57-1.55 (m, 6H); MS (ES+) m/z 506.4 (M+1).

### Example 324

### Synthesis of 2-(tert-butyl)-N-(2′-(rac-(syn)-4,4-difluoro-2-methylcyclohexyl)-3-fluoro-[2,4′-bipyridin]-3′-yl)pyrimidine-5-carboxamide

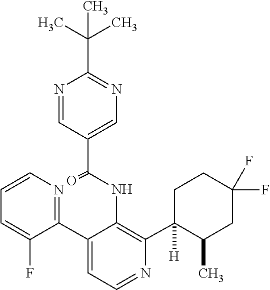 [SMILES: C[C@@H]1CC(F)(F)CC[C@@H]1c1nccc(-c2ncccc2F)c1NC(=O)c1cnc(C(C)(C)C)nc1]

### Step 1. Preparation of rac-tert-butyl (2′-(4,4-difluoro-6-methylcyclohex-1-en-1-yl)-3-fluoro-[2,4′-bipyridin]-3′-yl)carbamate

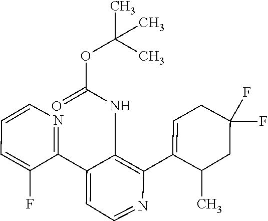 [SMILES: CC1CC(F)(F)CC=C1c1nccc(-c2ncccc2F)c1NC(=O)OC(C)(C)C]

A mixture of tert-butyl (2′-chloro-3-fluoro-[2,4′-bipyridin]-3′-yl)carbamate (2.0 g, 6.2 mmol) in dioxane (37 mL) was degassed with nitrogen for 10 minutes. To the reaction mixture was added N—[(Z)-(4,4-difluoro-2-methyl-cyclohexylidene)amino]-4-methyl-benzenesulfonamide (3.0 g, 9.5 mmol), tris(dibenzylideneacetone)dipalladium(0) (0.57 g, 0.62 mmol), tricyclohexylphosphine tetrafluoroborate (0.48 g, 1.3 mmol) and lithium tert-butoxide (1.5 g, 18.5 mmol). The reaction mixture was stirred at 110° C. for 18 h. After cooling to ambient temperature, the reaction mixture was diluted in ethyl acetate (500 mL) and washed with 1M sodium hydroxide (2×50 mL). The organic phase was washed with brine (50 mL), dried over anhydrous magnesium sulfate, filtered, and concentrated in vacuo. The residue was purified by column chromatography, eluting with a gradient of 5 to 80% of ethyl acetate in heptane, to afford the title compound as a mixture of compounds carried forward, colorless oil (2.4 g): MS (ES+) m/z 420.4 (M+1).

### Step 2. Preparation of rac-2′-(4,4-difluoro-6-methylcyclohex-1-en-1-yl)-3-fluoro-[2,4′-bipyridin]-3′-amine

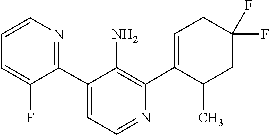 [SMILES: CC1CC(F)(F)CC=C1c1nccc(-c2ncccc2F)c1N]

A mixture of tert-butyl (2′-(4,4-difluoro-6-methylcyclohex-1-en-1-yl)-3-fluoro-[2,4′-bipyridin]-3′-yl)carbamate and 4M hydrochloric acid in 1,4-dioxane (28.3 mL) was heated to 40° C. for 3 h. After cooling to ambient temperature, the reaction mixture was diluted with ethyl acetate (500 mL) and 5M sodium hydroxide (50 mL). The layers were separated, and the organic layer was washed with brine (50 mL). The organic layer was dried over anhydrous magnesium sulfate, filtered, and concentrated in vacuo. The residue was a mixture of compounds used as is without further purification (1.8 g): MS (ES+) m/z 320.4 (M+1).

### Step 3. Preparation of 2′-(rac-(syn)-4,4-difluoro-2-methylcyclohexyl)-3-fluoro-[2,4′-bipyridin]-3′-amine

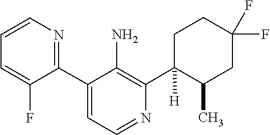 [SMILES: C[C@@H]1CC(F)(F)CC[C@@H]1c1nccc(-c2ncccc2F)c1N]

A mixture of rac-2′-(4,4-difluoro-6-methylcyclohex-1-en-1-yl)-3-fluoro-[2,4′-bipyridin]-3′-amine (1.6 g, 5.0 mmol) in ethanol (84 mL) and acetic acid (50 mL) was degassed with nitrogen for 10 minutes. To the reaction mixture was added ammonium formate (3.2 g, 50 mmol) and 10% palladium hydroxide on carbon (1.8 g). The reaction mixture was stirred at 80° C. for 1 h before being charged with ammonium formate (3.7 g, 59 mmol). The reaction mixture was stirred at 80° C. for 18 h before being charged with ammonium formate (3.7 g, 59 mmol) and 10% palladium hydroxide on carbon (1.4 g). The reaction mixture was stirred at 80° C. for a further 18 h before being charged with ammonium formate (2.4 g, 38 mmol) and 10% palladium hydroxide on carbon (1.4 g). The reaction mixture was stirred for 18 h at 80° C. After cooling to ambient temperature, the mixture was diluted in ethyl acetate (500 mL), filtered through a bed of Celite, and concentrated in vacuo. The residue was purified by column chromatography, eluting with a gradient of 10 to 50% of ethyl acetate in heptane to afford a mixture of compounds used without further purification (0.78 g): MS (ES+) m/z 322.4 (M+1).

### Step 4. Preparation of 2-(tert-butyl)-N-(2′-(rac-(syn)-4,4-difluoro-2-methylcyclohexyl)-3-fluoro-[2,4′-bipyridin]-3′-yl)pyrimidine-5-carboxamide

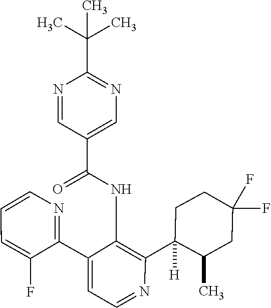 [SMILES: C[C@@H]1CC(F)(F)CC[C@@H]1c1nccc(-c2ncccc2F)c1NC(=O)c1cnc(C(C)(C)C)nc1]

To a mixture of 2′-(rac-(syn)-4,4-difluoro-2-methylcyclohexyl)-3-fluoro-[2,4′-bipyridin]-3′-amine (0.79 g, 2.5 mmol), 2-tert-butylpyrimidine-5-carboxylic acid (0.44 g, 2.5 mmol) and 2-chloro-1-methylpyridinium iodide (1.6 g, 6.2 mmol) was added anhydrous tetrahydrofuran (49 mL). The solution was heated at 70° C. for 1 min before N-ethyl-N-isopropylpropan-2-amine (3.2 g, 25 mmol) was added. The reaction mixture was stirred at 70° C. for 2 h. The reaction mixture was cooled to ambient temperature and diluted with ethyl acetate (125 mL) and washed with 1M sodium hydroxide (50 mL), and brine (50 mL). The organic layer was dried over anhydrous magnesium sulfate, filtered, and concentrated in vacuo. Purification of the residue by column chromatography, eluting with a gradient of 20 to 70% of ethyl acetate in heptane, followed by reverse phase column chromatography, eluting with a gradient of 10 to 60% of acetonitrile in water containing 0.5% formic acid, provided the title compound as a colorless solid (0.21 g, 17% yield): ¹H-NMR (400 MHz; DMSO-d₆) δ 10.41 (s, 1H), 8.94 (s, 2H), 8.67 (d, J=4.9 Hz, 1H), 8.48 (d, J=4.6 Hz, 1H), 7.87-7.82 (m, 1H), 7.50 (quintet, J=4.3 Hz, 2H), 3.50 (t, J=0.5 Hz, 1H), 2.51 (m, 1H), 2.29-2.25 (m, 2H), 2.09 (d, J=9.8 Hz, 1H), 1.94-1.84 (m, 3H), 1.37 (s, 9H), 0.75 (d, J=6.9 Hz, 3H); MS (ES+) m/z 484.4 (M+1).

### Example 325

### Synthesis of N-(2′-(4,4-difluorocyclohexyl)-3-fluoro-[2,4′-bipyridin]-3′-yl)-2-methoxypyrimidine-5-carboxamide

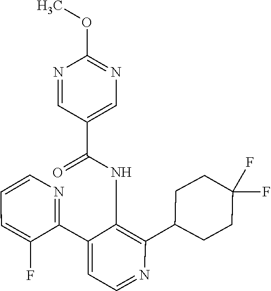 [SMILES: COc1ncc(C(=O)Nc2c(-c3ncccc3F)ccnc2C2CCC(F)(F)CC2)cn1]

To a mixture of 2′-(4,4-difluorocyclohexyl)-3-fluoro-[2,4′-bipyridin]-3′-amine (0.050 g, 0.16 mmol), 2-chloropyrimidine-5-carboxylic acid (0.052 g, 0.33 mmol) and 2-chloro-1-methylpyridinium iodide (0.10 g, 0.41 mmol) was added anhydrous tetrahydrofuran (3.3 mL). The solution was heated at 70° C. for 1 min before N-ethyl-N-isopropylpropan-2-amine (0.21 g, 1.6 mmol) was added. The reaction mixture was stirred at 70° C. for 18 h. The reaction mixture was cooled to 40° C. and diluted with methanol (4.0 mL) and 10M sodium hydroxide (2.0 mL). After stirring at 40° C. for 20 min the reaction mixture was diluted with ethyl acetate (100 mL). The organic layer was washed with 1M sodium hydroxide (50 mL), and brine (50 mL), dried over anhydrous magnesium sulfate, filtered, and concentrated in vacuo. Purification of the residue by column chromatography, eluting with a gradient of 15 to 100% of ethyl acetate in heptane, provided the title compound as a light brown solid (0.026 g, 35% yield): ¹H-NMR (400 MHz; DMSO-d₆) δ 10.27 (s, 1H), 8.87 (d, J=2.7 Hz, 2H), 8.66 (d, J=4.9 Hz, 1H), 8.46-8.44 (m, 1H), 7.82 (ddd, J=10.3, 8.5, 1.3 Hz, 1H), 7.51-7.46 (m, 2H), 3.98 (s, 3H), 3.22-3.19 (m, 1H), 2.14-2.06 (m, 2H), 1.98-1.85 (m, 6H); MS (ES+) m/z 444.2 (M+1).

### Example 326

### Synthesis of N-(2-(4,4-difluorocyclohexyl)-4-(2,5-difluorophenyl)pyridin-3-yl)-2-methoxypyrimidine-5-carboxamide

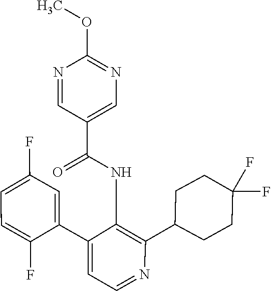 [SMILES: COc1ncc(C(=O)Nc2c(-c3cc(F)ccc3F)ccnc2C2CCC(F)(F)CC2)cn1]

To a mixture of 2-(4,4-difluorocyclohexyl)-4-(2,5-difluorophenyl)pyridin-3-amine (0.050 g, 0.15 mmol), 2-chloropyrimidine-5-carboxylic acid (0.049 g, 0.31 mmol) and 2-chloro-1-methylpyridinium iodide (0.098 g, 0.39 mmol) was added anhydrous tetrahydrofuran (3.1 mL). The solution was heated at 70° C. for 1 min before N-ethyl-N-isopropylpropan-2-amine (0.20 g, 1.5 mmol) was added. The reaction mixture was stirred at 70° C. for 18 h. The reaction mixture was cooled to 40° C. and diluted with methanol (4.0 mL) and 10M sodium hydroxide (2.0 mL). After stirring at 40° C. for 20 min the reaction mixture was diluted with ethyl acetate (100 mL). The organic layer was washed with 1M sodium hydroxide (50 mL), and brine (50 mL), dried over anhydrous magnesium sulfate, filtered, and concentrated in vacuo. Purification of the residue by column chromatography, eluting with 10 to 60% of ethyl acetate in heptane, provided the title compound as a colorless solid (0.048 g, 67% yield): ¹H-NMR (400 MHz; DMSO-d₆) δ 10.24 (s, 1H), 8.89 (s, 2H), 8.63 (d, J=4.9 Hz, 1H), 7.38 (dd, J=4.9, 0.8 Hz, 1H), 7.34 (dd, J=9.1, 4.6 Hz, 1H), 7.30-7.22 (m, 2H), 3.98 (s, 3H), 3.20-3.19 (m, 1H), 2.10-2.06 (m, 2H), 1.98-1.81 (m, 6H); MS (ES+) m/z 461.2 (M+1).

### Example 327

### Synthesis of rac-N-(2′-(5,5-difluorotetrahydro-2H-pyran-2-yl)-3-fluoro-[2,4′-bipyridin]-3′-yl)-2-ethoxypyrimidine-5-carboxamide

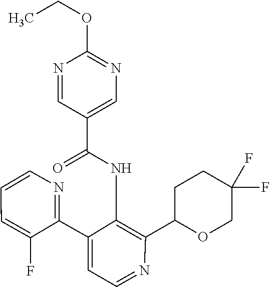 [SMILES: CCOc1ncc(C(=O)Nc2c(-c3ncccc3F)ccnc2C2CCC(F)(F)CO2)cn1]

To a mixture of rac-2′-(5,5-difluorotetrahydro-2H-pyran-2-yl)-3-fluoro-[2,4′-bipyridin]-3′-amine (0.13 g, 0.40 mmol), 2-chloropyrimidine-5-carboxylic acid (0.13 g, 0.81 mmol) and 2-chloro-1-methylpyridinium iodide (0.31 g, 1.2 mmol) was added anhydrous tetrahydrofuran (8.1 mL). The solution was heated at 50° C. for 1 min before N-ethyl-N-isopropylpropan-2-amine (0.52 g, 4.0 mmol) was added. The reaction mixture was stirred at 50° C. for 3 h. The reaction mixture was cooled to ambient temperature, diluted with ethyl acetate (100 mL) and washed with saturated potassium carbonate (50 mL). The organic layer was dried over anhydrous magnesium sulfate, filtered, and concentrated in vacuo. The residue was dissolved in anhydrous ethanol (8.1 mL) and 60% sodium hydride dispersion in mineral oil (0.16 g, 4.0 mmol) was added. The reaction mixture was heated to 40° C. for 1 h. The reaction mixture was diluted with ethyl acetate (100 mL), washed with brine (50 mL), dried over anhydrous magnesium sulfate, filtered, and concentrated in vacuo. Purification of the residue by column chromatography, eluting with a gradient of 20 to 100% of ethyl acetate in heptane, provided the title compound as a red solid (0.068 g, 35% yield): ¹H-NMR (400 MHz; CD₃CN) δ 9.07 (s, 1H), 8.84 (s, 2H), 8.67 (d, J=4.9 Hz, 1H), 8.47-8.45 (m, 1H), 7.66-7.61 (m, 1H), 7.59 (dt, J=4.7, 2.3 Hz, 1H), 7.42 (ddd, J=8.5, 4.5, 4.1 Hz, 1H), 4.94-4.90 (m, 1H), 4.47 (q, J=7.1 Hz, 2H), 4.07-4.00 (m, 1H), 3.83-3.73 (m, 1H), 2.38-2.30 (m, 2H), 2.22-2.10 (m, 2H), 1.41 (q, J=6.1 Hz, 3H); MS (ES+) m/z 460.2 (M+1).

### Example 328

### Synthesis of rac-N-(2′-(5,5-difluorotetrahydro-2H-pyran-2-yl)-3-fluoro-[2,4′-bipyridin]-3′-yl)-6-ethoxy-5-fluoronicotinamide

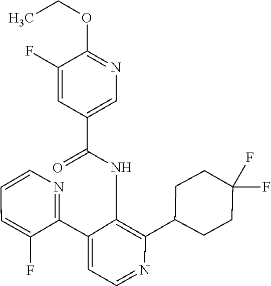 [SMILES: CCOc1ncc(C(=O)Nc2c(-c3ncccc3F)ccnc2C2CCC(F)(F)CC2)cc1F]

To a mixture of rac-2′-(5,5-difluorotetrahydro-2H-pyran-2-yl)-3-fluoro-[2,4′-bipyridin]-3′-amine (0.13 g, 0.40 mmol), 2,3-difluoropyridine-5-carboxylic acid (0.13 g, 0.81 mmol) and 2-chloro-1-methylpyridinium iodide (0.31 g, 1.2 mmol) was added anhydrous tetrahydrofuran (8.1 mL). The solution was heated at 50° C. for 1 min before N-ethyl-N-isopropylpropan-2-amine (0.52 g, 4.0 mmol) was added. The reaction mixture was stirred at 50° C. for 3 h. The reaction mixture was cooled to ambient temperature, diluted with ethyl acetate (100 mL) and washed with saturated potassium carbonate (50 mL). The organic layer was dried over anhydrous magnesium sulfate, filtered, and concentrated in vacuo. The residue was purified by column chromatography, eluting with a gradient of 15 to 100% of ethyl acetate in heptane. The purified material was dissolved in anhydrous ethanol (8.1 mL) and 60% sodium hydride dispersion in mineral oil (0.16 g, 4.0 mmol) was added. The reaction mixture was stirred at ambient temperature for 18 h. The reaction mixture was diluted with ethyl acetate (100 mL), washed with brine (50 mL), dried over anhydrous magnesium sulfate, filtered, and concentrated in vacuo. Purification of the residue by column chromatography, eluting with a gradient of 20 to 100% of ethyl acetate in heptane, provided the title compound as an orange solid (0.059 g, 29% yield): ¹H-NMR (400 MHz; CD₃CN) δ 9.06 (d, J=5.4 Hz, 1H), 8.67 (d, J=4.9 Hz, 1H), 8.47-8.44 (m, 1H), 8.35 (d, J=1.8 Hz, 1H), 7.75 (dd, J=10.9, 1.8 Hz, 1H), 7.65-7.60 (m, 1H), 7.60-7.58 (m, 1H), 7.42 (ddd, J=8.5, 4.5, 4.1 Hz, 1H), 4.93-4.90 (m, 1H), 4.51 (q, J=7.1 Hz, 2H), 4.04 (dtd, J=13.4, 5.7, 2.5 Hz, 1H), 3.83-3.72 (m, 1H), 2.37-2.30 (m, 2H), 2.16-2.10 (m, 2H), 1.45-1.40 (m, 3H); MS (ES+) m/z 477.2 (M+1).

### Example 329

### Synthesis of N-(2′-(rac-(syn)-4,4-difluoro-2-methylcyclohexyl)-3-fluoro-[2,4′-bipyridin]-3′-yl)-2-methoxypyrimidine-5-carboxamide

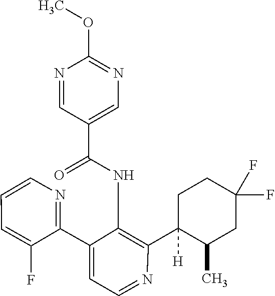 [SMILES: COc1ncc(C(=O)Nc2c(-c3ncccc3F)ccnc2[C@H]2CCC(F)(F)C[C@H]2C)cn1]

To a mixture of 2′-(rac-(syn)-4,4-difluoro-2-methylcyclohexyl)-3-fluoro-[2,4′-bipyridin]-3′-amine (0.15 g, 0.47 mmol), 2-chloropyrimidine-5-carboxylic acid (0.15 g, 0.93 mmol) and 2-chloro-1-methylpyridinium iodide (0.27 g, 1.1 mmol) was added anhydrous tetrahydrofuran (9.3 mL). The solution was heated at 70° C. for 1 min before N-ethyl-N-isopropylpropan-2-amine (0.60 g, 4.7 mmol) was added. The reaction mixture was stirred at 70° C. for 18 h. The reaction mixture was diluted with ethyl acetate (125 mL) and washed with 1M sodium hydroxide (50 mL), and brine (50 mL). The organic layer was dried over anhydrous magnesium sulfate, filtered, and concentrated in vacuo. The residue was dissolved in methanol (10 mL), and 60% sodium hydride dispersion in mineral oil (0.19 g, 4.7 mmol) was added. The reaction mixture was stirred at ambient temperature for 1 h. The reaction mixture was diluted with ethyl acetate (200 mL). The organic layer was washed with brine (50 mL), dried over anhydrous magnesium sulfate, filtered, and concentrated in vacuo. Purification of the residue by column chromatography, eluting with a gradient of 10 to 60% of ethyl acetate in heptane, provided the title compound as a colorless solid (0.047 g, 21% yield): ¹H-NMR (400 MHz; DMSO-d₆) δ 10.29 (s, 1H), 8.85 (s, 2H), 8.66 (d, J=4.9 Hz, 1H), 8.45 (dt, J=3.0, 1.5 Hz, 1H), 7.82 (ddd, J=10.1, 8.6, 1.3 Hz, 1H), 7.48 (quintet, J=4.2 Hz, 2H), 3.97 (s, 3H), 3.49-3.48 (m, 1H), 2.52-2.46 (m, 1H), 2.33-2.23 (m, 2H), 2.14-2.08 (m, 1H), 1.97-1.82 (m, 3H), 0.75 (d, J=7.0 Hz, 3H); MS (ES+) m/z 458.2 (M+1).

### Example 330

### Synthesis of rac-N-(2-(5,5-difluorotetrahydro-2H-pyran-2-yl)-4-(2,4,5-trifluorophenyl)pyridin-3-yl)-2-isopropylpyrimidine-5-carboxamide

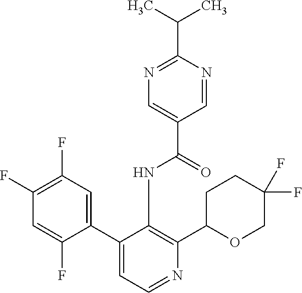 [SMILES: CC(C)c1ncc(C(=O)Nc2c(-c3cc(F)c(F)cc3F)ccnc2C2CCC(F)(F)CO2)cn1]

### Step 1. Preparation of tert-butyl (2-chloro-4-(2,4,5-trifluorophenyl)pyridin-3-yl)carbamate

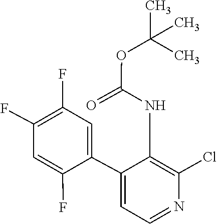 [SMILES: CC(C)(C)OC(=O)Nc1c(-c2cc(F)c(F)cc2F)ccnc1Cl]

A mixture of tert-butyl N-(2-chloro-4-iodo-3-pyridyl)carbamate (1.0 g, 2.8 mmol), water (1.7 mL), and 1,4-dioxane (16 mL) was sparged with nitrogen for 10 min. To the mixture was added 2,4,5-trifluorophenylboronic acid (0.52 g, 3.0 mmol), [1,1′-bis(diphenylphosphino)ferrocene]dichloropalladium(II) dichloromethane complex (0.24 g, 0.28 mmol), and potassium carbonate (1.1 g, 8.5 mmol). The reaction mixture was sealed and heated to 80° C. for 3 h. The reaction mixture was cooled to ambient temperature and diluted with ethyl acetate (100 mL). The reaction mixture was washed with saturated ammonium chloride solution (2×50 mL). The organic layer was dried over anhydrous magnesium sulfate, filtered, and concentrated in vacuo. Purification of the residue by column chromatography, eluting with a gradient of 5 to 25% of ethyl acetate in heptane, provided the title compound as a colorless solid (0.98 g, 97% yield): MS (ES+) m/z 359.2 (M+1), 361.2 (M+1).

### Step 2. Preparation of rac-2-(5,5-difluorotetrahydro-2H-pyran-2-yl)-4-(2,4,5-trifluorophenyl)pyridin-3-amine

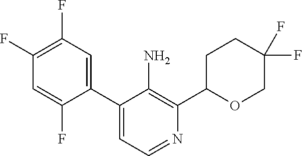 [SMILES: Nc1c(-c2cc(F)c(F)cc2F)ccnc1C1CCC(F)(F)CO1]

To tert-butyl (2-chloro-4-(2,4,5-trifluorophenyl)pyridin-3-yl)carbamate (0.98 g, 2.7 mmol) was added tetrahydropyran-2-carboxylic acid (0.59 g, 3.6 mmol), (4,4″-di-t-butyl-2,2″-bipyridine)bis[3,5-difluoro-2-[5-trifluoromethyl-2-pyridinyl-kN)phenyl-kC]iridium(III) hexafluorophosphate (0.031 g, 0.027 mmol), dichloro(dimethoxyethane)nickel (0.060 g, 0.27 mmol), 4-tert-butyl-2-(4-tert-butyl-2-pyridyl)pyridine (0.11 g, 0.41 mmol), and cesium carbonate (1.2 g, 3.8 mmol) was split into two vials before anhydrous N,N-dimethylformamide (35 mL) was added to each. The headspace of the vials was replaced with nitrogen, the vials were sealed, and the reaction mixtures were stirred in front of Kessil PR160L lights (440 nm) for 24 h. The two reaction mixtures were mixed, diluted with ethyl acetate (600 mL), and washed with 1M sodium hydroxide (25 mL), water (3×50 mL), and saturated ammonium chloride solution (50 mL). The organic layer was dried over anhydrous magnesium sulfate, filtered, and concentrated in vacuo. The residue was dissolved in 4M hydrogen chloride in 1,4-dioxane (14 mL) and stirred at 40° C. for 1 h. After cooling to ambient temperature, the reaction mixture was diluted with ethyl acetate (250 mL) and 5M sodium hydroxide (25 mL). The layers were separated, and the organic layer was washed with saturated ammonium chloride (50 mL). The organic layer was dried over anhydrous magnesium sulfate, filtered, and concentrated in vacuo. Purification of the residue by column chromatography, eluting with a gradient of 5 to 50% of ethyl acetate in heptane, afforded the title compound as a colorless solid (0.64 g, 68% yield): MS (ES+) m/z 345.2 (M+1).

### Step 3. Preparation of rac-N-(2-(5,5-difluorotetrahydro-2H-pyran-2-yl)-4-(2,4,5-trifluorophenyl)pyridin-3-yl)-2-isopropylpyrimidine-5-carboxamide

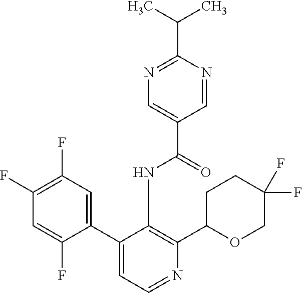 [SMILES: CC(C)c1ncc(C(=O)Nc2c(-c3cc(F)c(F)cc3F)ccnc2C2CCC(F)(F)CO2)cn1]

To a mixture of rac-2-(5,5-difluorotetrahydro-2H-pyran-2-yl)-4-(2,4,5-trifluorophenyl)pyridin-3-amine (0.32 g, 0.93 mmol), 2-isopropylpyrimidine-5-carboxylic acid (0.31 g, 1.9 mmol) and 2-chloro-1-methylpyridinium iodide (0.59 g, 2.3 mmol) was added anhydrous tetrahydrofuran (19 mL). The solution was heated at 55° C. for 1 min before N-ethyl-N-isopropylpropan-2-amine (1.2 g, 9.3 mmol) was added. The reaction mixture was stirred at 55° C. for 18 h. The reaction mixture was cooled to ambient temperature and diluted with ethyl acetate (300 mL) and washed with water (3×50 mL), and saturated ammonium chloride solution (2×50 mL). The organic layer was dried over anhydrous magnesium sulfate, filtered, and concentrated in vacuo. The residue was dissolved in methanol (10 mL) and 5M sodium hydroxide (1.0 mL). The reaction mixture was heated to 40° C. for 1 h. The reaction mixture was diluted with ethyl acetate (250 mL), washed with saturated ammonium chloride (2×50 mL), dried over anhydrous magnesium sulfate, filtered, and concentrated in vacuo. The residue was purified by column chromatography, eluting with a gradient of 10 to 60% of ethyl acetate in heptane, to provide the title compound as a colorless solid (0.31 g, 67% yield): ¹H-NMR (400 MHz; DMSO-d₆) δ 10.39 (s, 1H), 9.01 (s, 2H), 8.69 (d, J=4.9 Hz, 1H), 7.74-7.67 (m, 1H), 7.61-7.56 (m, 1H), 7.53 (d, J=4.9 Hz, 1H), 4.95-4.92 (m, 1H), 3.92-3.90 (m, 1H), 3.83 (t, J=15.8 Hz, 1H), 3.21 (dt, J=13.8, 6.9 Hz, 1H), 2.31-2.23 (m, 3H), 1.97-1.94 (m, 1H), 1.29 (t, J=5.9 Hz, 6H); MS (ES+) m/z 493.2 (M+1).

### Example 331

### Synthesis of rac-N-(2-(5,5-difluorotetrahydro-2H-pyran-2-yl)-4-(2,4,5-trifluorophenyl)pyridin-3-yl)-2-isopropoxypyrimidine-5-carboxamide

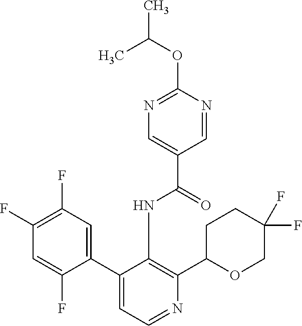 [SMILES: CC(C)Oc1ncc(C(=O)Nc2c(-c3cc(F)c(F)cc3F)ccnc2C2CCC(F)(F)CO2)cn1]

### Step 1. Preparation of a mixture of rac-2-chloro-N-(2-(5,5-difluorotetrahydro-2H-pyran-2-yl)-4-(2,4,5-trifluorophenyl)pyridin-3-yl)pyrimidine-5-carboxamide and rac-2-chloro-N-(2-chloropyrimidine-5-carbonyl)-N-(2-(5,5-difluorotetrahydro-2H-pyran-2-yl)-4-(2,4,5-trifluorophenyl)pyridin-3-yl)pyrimidine-5-carboxamide

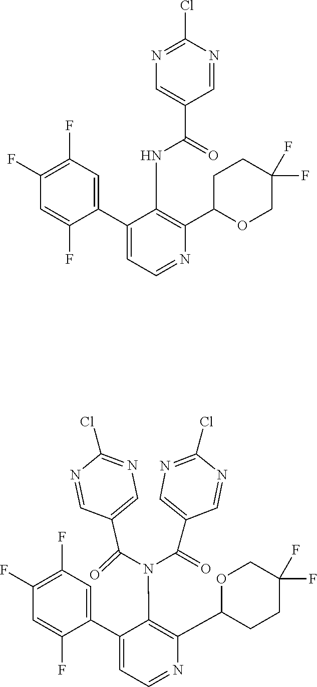 [SMILES: O=C(Nc1c(-c2cc(F)c(F)cc2F)ccnc1C1CCC(F)(F)CO1)c1cnc(Cl)nc1 ; O=C(c1cnc(Cl)nc1)N(C(=O)c1cnc(Cl)nc1)c1c(-c2cc(F)c(F)cc2F)ccnc1C1CCC(F)(F)CO1]

To a mixture of rac-2-(5,5-difluorotetrahydro-2H-pyran-2-yl)-4-(2,4,5-trifluorophenyl)pyridin-3-amine (0.32 g, 0.93 mmol), 2-chloropyrimidine-5-carboxylic acid (0.29 g, 1.9 mmol) and 2-chloro-1-methylpyridinium iodide (0.59 g, 2.3 mmol) was added anhydrous tetrahydrofuran (19 mL). The solution was heated at 55° C. for 1 min and N-ethyl-N-isopropylpropan-2-amine (1.2 g, 9.3 mmol) was added. The reaction mixture was stirred at 55° C. for 18 h. The reaction mixture was cooled to ambient temperature, diluted with ethyl acetate (300 mL), and washed with saturated ammonium chloride solution (2×50 mL). The organic layer was dried over anhydrous magnesium sulfate, filtered, and concentrated in vacuo. The residue was purified by column chromatography, eluting with a gradient of 10 to 60% of ethyl acetate in heptane, to provide the title compounds as a mixture (0.20 g): MS (ES+) m/z 485.4 (M+1), 487.4 (M+1), 625.4 (M+1), 627.4 (M+1).

### Step 2. Preparation of rac-N-(2-(5,5-difluorotetrahydro-2H-pyran-2-yl)-4-(2,4,5-trifluorophenyl)pyridin-3-yl)-2-isopropoxypyrimidine-5-carboxamide

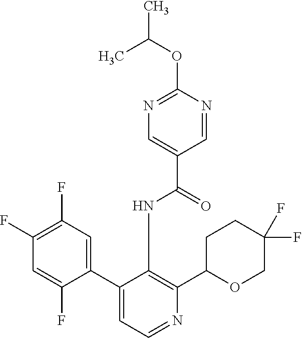 [SMILES: CC(C)Oc1ncc(C(=O)Nc2c(-c3cc(F)c(F)cc3F)ccnc2C2CCC(F)(F)CO2)cn1]

To a mixture of rac-2-chloro-N-(2-(5,5-difluorotetrahydro-2H-pyran-2-yl)-4-(2,4,5-trifluorophenyl)pyridin-3-yl)pyrimidine-5-carboxamide and rac-2-chloro-N-(2-chloropyrimidine-5-carbonyl)-N-(2-(5,5-difluorotetrahydro-2H-pyran-2-yl)-4-(2,4,5-trifluorophenyl)pyridin-3-yl)pyrimidine-5-carboxamide-1-methane (0.20 g) in anhydrous N,N-dimethylformamide (7.3 mL) was added 2-propanol (7.3 mL) and 60% sodium hydride dispersion in mineral oil (0.087 g, 3.6 mmol). The solution was heated to 40° C. for 18 h. The reaction mixture was diluted with ethyl acetate (350 mL) and washed with saturated ammonium chloride solution (50 mL), water (2×50 mL), and saturated ammonium chloride (50 mL). The organic layer was dried over anhydrous magnesium sulfate, filtered, and concentrated in vacuo. Purification of the residue by column chromatography, eluting with a gradient of 10 to 75% of ethyl acetate in heptane, followed by reverse phase column chromatography, eluting with a gradient of 40 to 90% of acetonitrile in water, provided the title compound as a colorless solid (0.084 g, 22% yield): ¹H-NMR (400 MHz; DMSO-d₆) δ 10.22 (s, 1H), 8.90 (d, J=4.2 Hz, 2H), 8.67 (d, J=4.9 Hz, 1H), 7.69 (ddd, J=10.6, 9.9, 6.8 Hz, 1H), 7.58-7.54 (m, 1H), 7.52 (d, J=5.0 Hz, 1H), 5.28 (quintet, J=6.2 Hz, 1H), 4.93-4.90 (m, 1H), 3.92-3.90 (m, 1H), 3.81 (t, J=15.8 Hz, 1H), 2.31-2.24 (m, 3H), 1.96 (dd, J=9.6, 2.3 Hz, 1H), 1.34 (d, J=6.2 Hz, 6H); MS (ES+) m/z 509.2 (M+1).

### Example 332

### Synthesis of N-(4-(2,5-difluorophenyl)-2-(tetrahydro-2H-pyran-4-yl)pyridin-3-yl)-2-isopropylpyrimidine-5-carboxamide

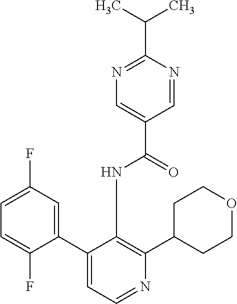 [SMILES: CC(C)c1ncc(C(=O)Nc2c(-c3cc(F)ccc3F)ccnc2C2CCOCC2)cn1]

### Step 1. Preparation of 4-(2,5-difluorophenyl)-2-(tetrahydro-2H-pyran-4-yl)pyridin-3-amine

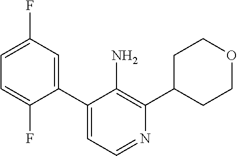 [SMILES: Nc1c(-c2cc(F)ccc2F)ccnc1C1CCOCC1]

To a mixture of tert-butyl (2-chloro-4-(2,5-difluorophenyl)pyridin-3-yl)carbamate (0.50 g, 1.5 mmol) was added tetrahydropyran-4-carboxylic acid (0.31 g, 2.3 mmol), (4,4″-di-t-butyl-2,2″-bipyridine)bis[3,5-difluoro-2-[5-trifluoromethyl-2-pyridinyl-kN)phenyl-kC]iridium(II) hexafluorophosphate (0.016 g, 0.015 mmol), dichloro(dimethoxyethane)nickel (0.032 g, 0.15 mmol), 4-tert-butyl-2-(4-tert-butyl-2-pyridyl)pyridine (0.059 g, 0.22 mmol), and cesium carbonate (0.84 g, 2.6 mmol) was added anhydrous N,N-dimethylformamide (29 mL). The headspace of the vial was replaced with nitrogen, the vial was sealed, and the reaction mixture was stirred in front of Kessil PR160L lights (440 nm) for 18 h. The reaction mixture was diluted with ethyl acetate (200 mL) and washed with water (5×50 mL), and saturated ammonium chloride (2×50 mL). The organic layer was dried over anhydrous magnesium sulfate, filtered, and concentrated in vacuo. The residue was dissolved in 4M hydrogen chloride in 1,4-dioxane (7.3 mL) and stirred at ambient temperature for 2 h. After cooling to ambient temperature, the reaction mixture was diluted with ethyl acetate (300 mL) and 5M sodium hydroxide (10 mL). The layers were separated, and the organic layer was washed with 1M sodium hydroxide (50 mL), and saturated ammonium chloride (50 mL). The organic layer was dried over anhydrous magnesium sulfate, filtered, and concentrated in vacuo. Purification of the residue by column chromatography, eluting with a gradient of 10 to 60% of ethyl acetate in heptane, afforded the title compound as a yellow solid (0.33 g, 76% yield): MS (ES+) m/z 291.4 (M+1).

### Step 2. Preparation of N-(4-(2,5-difluorophenyl)-2-(tetrahydro-2H-pyran-4-yl)pyridin-3-yl)-2-isopropylpyrimidine-5-carboxamide

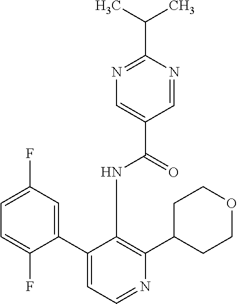 [SMILES: CC(C)c1ncc(C(=O)Nc2c(-c3cc(F)ccc3F)ccnc2C2CCOCC2)cn1]

To a mixture of 4-(2,5-difluorophenyl)-2-(tetrahydro-2H-pyran-4-yl)pyridin-3-amine (0.16 g, 0.55 mmol), 2-isopropylpyrimidine-5-carboxylic acid (0.14 g, 0.83 mmol) and 2-chloro-1-methylpyridinium iodide (0.32 g, 1.3 mmol) was added anhydrous tetrahydrofuran (11 mL). The solution was heated at 50° C. for 1 min before N-ethyl-N-isopropylpropan-2-amine (0.71 g, 5.5 mmol) was added. The reaction mixture was stirred at 50° C. for 18 h. The reaction mixture was cooled to ambient temperature and diluted with ethyl acetate (100 mL) and washed with 1M sodium hydroxide (50 mL), and saturated ammonium chloride solution (50 mL). The organic layer was dried over anhydrous magnesium sulfate, filtered, and concentrated in vacuo. The residue was purified by column chromatography, eluting with a gradient of 10 to 80% of ethyl acetate in heptane, to provide the title compound as a colorless solid (0.11 g, 46% yield): ¹H-NMR (400 MHz; DMSO-d₆) δ 10.39 (s, 1H), 8.96 (s, 2H), 8.66 (d, J=4.9 Hz, 1H), 7.36 (dt, J=12.3, 4.2 Hz, 2H), 7.27 (dtd, J=12.8, 6.6, 3.4 Hz, 2H), 3.94-3.91 (m, 2H), 3.43-3.37 (m, 2H), 3.35-3.26 (m, 1H), 3.20 (dt, J=13.8, 6.9 Hz, 1H), 1.92-1.88 (m, 2H), 1.64-1.61 (m, 2H), 1.29 (d, J=6.9 Hz, 6H); MS (ES+) m/z 439.2 (M+1).

### Example 333

### Synthesis of 3-(2,2-difluoroethoxy)-N-(4-(2,5-difluorophenyl)-2-(tetrahydro-2H-pyran-4-yl)pyridin-3-yl)isoxazole-5-carboxamide

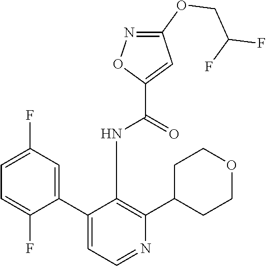 [SMILES: O=C(Nc1c(-c2cc(F)ccc2F)ccnc1C1CCOCC1)c1cc(OCC(F)F)no1]

To a mixture of 4-(2,5-difluorophenyl)-2-(tetrahydro-2H-pyran-4-yl)pyridin-3-amine (0.16 g, 0.55 mmol), 3-(2,2-difluoroethoxy)isoxazole-5-carboxylic acid (0.16 g, 0.83 mmol) and 2-chloro-1-methylpyridinium iodide (0.32 g, 1.3 mmol) was added anhydrous tetrahydrofuran (11 mL). The solution was heated at 50° C. for 1 min before N-ethyl-N-isopropylpropan-2-amine (0.71 g, 5.5 mmol) was added. The reaction mixture was stirred at 50° C. for 1 h. The reaction mixture was diluted with methanol (5 mL) and 5M sodium hydroxide (1 mL) and heated to 50° C. for 20 min. The reaction mixture was cooled to ambient temperature and diluted with ethyl acetate (100 mL) and washed with 1M sodium hydroxide (50 mL), and saturated ammonium chloride solution (2×50 mL). The organic layer was dried over anhydrous magnesium sulfate, filtered, and concentrated in vacuo. The residue was purified by column chromatography, eluting with a gradient of 10 to 75% of ethyl acetate in heptane, to provide the title compound as a colorless solid (0.17 g, 64% yield): ¹H-NMR (400 MHz; DMSO-d₆) δ 10.70 (s, 1H), 8.66 (d, J=4.9 Hz, 1H), 7.39-7.28 (m, 3H), 7.22 (ddd, J=8.8, 5.6, 3.1 Hz, 1H), 7.00 (d, J=3.3 Hz, 1H), 6.42 (tt, J=53.9, 3.2 Hz, 1H), 4.62-4.53 (m, 2H), 3.95-3.92 (m, 2H), 3.41-3.33 (m, 2H), 3.25-3.18 (m, 1H), 1.94-1.83 (m, 2H), 1.62-1.59 (m, 2H); MS (ES+) m/z 466.2 (M+1). Example 334, 335, 336, 337, and 338

### Synthesis of N-(4-(2,5-difluorophenyl)-2-((3S,4R)-3-methyltetrahydro-2H-pyran-4-yl)pyridin-3-yl)-2-isopropylpyrimidine-5-carboxamide P1-P4////

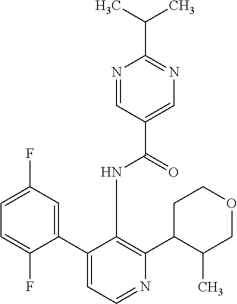 [SMILES: CC(C)c1ncc(C(=O)Nc2c(-c3cc(F)ccc3F)ccnc2C2CCOCC2C)cn1]

### Step 1. Preparation of rac-4-(2,5-difluorophenyl)-2-(3-methyl-3,6-dihydro-2H-pyran-4-yl)pyridin-3-amine

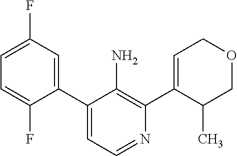 [SMILES: CC1COCC=C1c1nccc(-c2cc(F)ccc2F)c1N]

A mixture of tert-butyl (2′-chloro-3-fluoro-[2,4′-bipyridin]-3′-yl)carbamate (2.0 g, 5.9 mmol) in dioxane (36 mL) was degassed with nitrogen for 10 minutes. To the reaction mixture was added 4-methyl-N—[(Z)-(3-methyltetrahydropyran-4-ylidene)amino]benzenesulfonamide (1.7 g, 5.9 mmol), tris(dibenzylideneacetone)dipalladium(0) (0.54 g, 0.59 mmol), tricyclohexylphosphine tetrafluoroborate (0.45 g, 1.2 mmol) and lithium tert-butoxide (1.4 g, 18 mmol). The reaction mixture was stirred at 110° C. for 18 h. After cooling to ambient temperature, the reaction mixture was diluted in ethyl acetate (350 mL) and washed with saturated ammonium chloride (2×50 mL). The organic layer was dried over anhydrous magnesium sulfate, filtered, and concentrated in vacuo. The residue was purified by column chromatography, eluting with a gradient of 10 to 60% of ethyl acetate in heptane. The aniline was dissolved in 4M hydrogen chloride in 1,4-dioxane (29 mL) and heated to 40° C. for 2 h. After cooling to ambient temperature, the reaction mixture was diluted in ethyl acetate (300 mL) and washed with 5M sodium hydroxide (100 mL). The layers were separated, and the organic layer was dried over anhydrous magnesium sulfate, filtered, and concentrated in vacuo. The residue was purified by column chromatography, eluting with a gradient of 10 to 75% of ethyl acetate in heptane to afford the title compound as a colorless oil (0.94 g, 53% yield): ¹H-NMR (400 MHz; CDCl₃) δ 8.10 (t, J=3.7 Hz, 1H), 7.23-7.17 (m, 1H), 7.16-7.11 (m, 2H), 6.95 (d, J=4.8 Hz, 1H), 6.07 (dd, J=3.6, 1.7 Hz, 1H), 4.39 (ddd, J=17.0, 2.8, 1.9 Hz, 1H), 4.27 (dt, J=17.0, 2.6 Hz, 1H), 3.98 (dd, J=11.1, 4.0 Hz, 1H), 3.77 (dd, J=11.1, 3.7 Hz, 1H), 3.00-2.97 (m, 1H), 1.05 (d, J=7.1 Hz, 3H); MS (ES+) m/z 303.2 (M+1).

### Step 2. Preparation of 4-(2,5-difluorophenyl)-2-(rac-(syn and anti) 3-methyltetrahydro-2H-pyran-4-yl)pyridin-3-amine

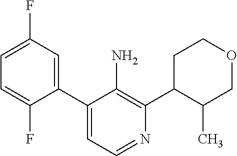 [SMILES: CC1COCCC1c1nccc(-c2cc(F)ccc2F)c1N]

A mixture of rac-4-(2,5-difluorophenyl)-2-(3-methyl-3,6-dihydro-2H-pyran-4-yl)pyridin-3-amine (0.94 g, 3.1 mmol) in ethanol (31 mL) and acetic acid (0.22 mL) was degassed with nitrogen for 10 minutes. To the reaction mixture was added ammonium formate (5.9 g, 93 mmol) and 10% palladium hydroxide on carbon (0.87 g). The reaction mixture was stirred at 80° C. for 1 h before being charged with ammonium formate (5.9 g, 93 mmol) and 10% palladium hydroxide on carbon (0.87 g). The reaction mixture was stirred at 80° C. for 18 h. After cooling to ambient temperature, the mixture was diluted in ethyl acetate (250 mL), filtered through a bed of Celite, and concentrated in vacuo. The residue was purified by column chromatography, eluting with a gradient of 12 to 75% of ethyl acetate in heptane, to afford a mixture of stereoisomers used as is without further purification (0.75 g, 79% yield): MS (ES+) m/z 305.2 (M+1).

### Step 3. Preparation of N-(4-(2,5-difluorophenyl)-3-methyltetrahydro-2H-pyran-4-yl)pyridin-3-yl)-2-isopropylpyrimidine-5-carboxamide

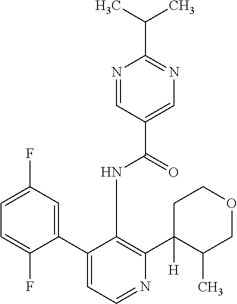 [SMILES: CC(C)c1ncc(C(=O)Nc2c(-c3cc(F)ccc3F)ccnc2C2CCOCC2C)cn1]

To a mixture of 4-(2,5-difluorophenyl)-2-(rac-(syn and anti)-3-methyltetrahydro-2H-pyran-4-yl)pyridin-3-amine (0.37 g, 1.2 mmol), 2-isopropylpyrimidine-5-carboxylic acid (0.31 g, 1.8 mmol) and 2-chloro-1-methylpyridinium iodide (0.72 g, 2.8 mmol) was added anhydrous tetrahydrofuran (25 mL). The solution was heated at 50° C. for 1 min before N-ethyl-N-isopropylpropan-2-amine (1.6 g, 12 mmol) was added. The reaction mixture was stirred at 50° C. for 18 h. The reaction mixture was diluted with methanol (5 mL), and 5M sodium hydroxide (2 mL) and heated to 40° C. for 30 min. The reaction mixture was cooled to ambient temperature, diluted with ethyl acetate (100 mL) and washed with 1M sodium hydroxide (50 mL), and saturated ammonium chloride (50 mL). The organic layer was dried over anhydrous magnesium sulfate, filtered, and concentrated in vacuo. Purification of the residue by column chromatography, eluting with a gradient of 10 to 80% of ethyl acetate in heptane, provided the title compound as a colorless solid.

The mixture of stereoisomers was subjected to:
Conditions A: chiral SFC (column: DAICEL CHIRALPAK IC (250 mm×30 mm, 10 μm), eluting with 20% of isopropanol containing 0.1% ammonium hydroxide in supercritical carbon dioxide, followed by,
Conditions B: chiral SFC (column: Phenomenex-Cellulose-2 (250 mm×30 mm, 10 μm), eluting with 20% of ethanol containing 0.1% ammonium hydroxide in supercritical carbon dioxide, to afford the four stereoisomers:
P1 (retention time=1.294 min, Conditions A) as a colorless solid (0.053 g, 10% yield, 99% ee): ¹H-NMR (400 MHz; CDCl₃) δ 8.92 (s, 2H), 8.71 (d, J=4.8 Hz, 1H), 7.52 (s, 1H), 7.23 (d, J=4.8 Hz, 1H), 7.16-7.02 (m, 3H), 4.23-4.10 (m, 1H), 3.83 (dd, J=11.2, 1.6 Hz, 1H), 3.64-3.56 (m, 1H), 3.55-3.49 (m, 1H), 3.45 (td, J=11.2, 3.6 Hz, 1H), 3.29 (quin, J=6.8 Hz, 1H), 2.73-2.53 (m, 1H), 2.09-1.97 (m, 1H), 1.59-1.50 (m, 1H), 1.38 (d, J=6.8 Hz, 6H), 0.92 (d, J=7.2 Hz, 3H); MS (ES+) m/z 466.4 (M+1).
P2 (retention time=1.418 min, Conditions A) as a colorless solid (0.052 g, 9% yield, 96% ee): ¹H-NMR (400 MHz; CDCl₃) δ 8.92 (s, 2H), 8.71 (d, J=4.8 Hz, 1H), 7.51 (s, 1H), 7.23 (d, J=4.8 Hz, 1H), 7.17-7.00 (m, 3H), 4.25-4.08 (m, 1H), 3.83 (dd, J=11.2, 2.0 Hz, 1H), 3.64-3.56 (m, 1H), 3.55-3.50 (m, 1H), 3.45 (td, J=11.2, 4.0 Hz, 1H), 3.29 (quin, J=6.8 Hz, 1H), 2.75-2.55 (m, 1H), 2.03 (d, J=2.4 Hz, 1H), 1.60-1.52 (m, 1H), 1.38 (d, J=6.8 Hz, 6H), 0.92 (d, J=7.2 Hz, 3H); MS (ES+) m/z 466.4 (M+1).
P3 (retention time=1.169 min, Conditions B) as a colorless solid (0.030 g, 5% yield, 98% ee): ¹H-NMR (400 MHz; DMSO-d₆) δ 10.38 (s, 1H), 8.94 (s, 2H), 8.66 (d, J=4.8 Hz, 1H), 7.40-7.31 (m, 2H), 7.30-7.22 (m, 2H), 3.91 (d, J=9.2 Hz, 1H), 3.82 (dd, J=11.2, 4.4 Hz, 1H), 3.41-3.34 (m, 1H), 3.20 (td, J=13.6, 7.2 Hz, 1H), 3.02 (t, J=11.2 Hz, 1H), 2.95-2.84 (m, 1H), 2.25-2.06 (m, 1H), 1.86-1.73 (m, 1H), 1.73-1.63 (m, 1H), 1.29 (d, J=6.8 Hz, 6H), 0.51 (d, J=5.2 Hz, 3H); MS (ES+) m/z 466.4 (M+1).
P4 (retention time=1.316 min, Conditions B) as a colorless solid (0.029 g, 5% yield, 99% ee): ¹H-NMR (400 MHz; DMSO-d₆) δ 10.36 (s, 1H), 8.94 (s, 2H), 8.66 (d, J=4.8 Hz, 1H), 7.40-7.32 (m, 2H), 7.30-7.21 (m, 2H), 3.95-3.87 (m, 1H), 3.82 (dd, J=11.2, 4.4 Hz, 1H), 3.41-3.34 (m, 1H), 3.20 (td, J=13.6, 6.8 Hz, 1H), 3.02 (t, J=11.2 Hz, 1H), 2.95-2.82 (m, 1H), 2.26-2.08 (m, 1H), 1.85-1.73 (m, 1H), 1.72-1.63 (m, 1H), 1.29 (d, J=6.8 Hz, 6H), 0.51 (d, J=5.2 Hz, 3H); MS (ES+) m/z 466.4 (M+1).

### Example 339, 340, 341, and 342

### Synthesis of 3-(2,2-difluoroethoxy)-N-(4-(2,5-difluorophenyl)-3-methyltetrahydro-2H-pyran-4-yl)pyridin-3-yl)isoxazole-5-carboxamide

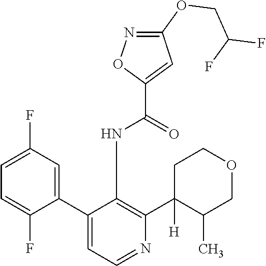 [SMILES: CC1COCCC1c1nccc(-c2cc(F)ccc2F)c1NC(=O)c1cc(OCC(F)F)no1]

To a mixture of 4-(2,5-difluorophenyl)-2-(rac-(syn and anti)-3-methyltetrahydro-2H-pyran-4-yl)pyridin-3-amine (0.37 g, 1.2 mmol), 3-(2,2-difluoroethoxy)isoxazole-5-carboxylic acid (0.36 g, 1.8 mmol) and 2-chloro-1-methylpyridinium iodide (0.72 g, 2.8 mmol) was added anhydrous tetrahydrofuran (25 mL). The solution was heated at 50° C. for 1 min before N-ethyl-N-isopropylpropan-2-amine (1.6 g, 12 mmol) was added. The reaction mixture was stirred at 50° C. for 18 h. The reaction mixture was diluted with methanol (5 mL), and 5M sodium hydroxide (2 mL) and heated to 40° C. for 30 min. The reaction mixture was cooled to ambient temperature and diluted with ethyl acetate (100 mL) and washed with 1M sodium hydroxide (50 mL), and saturated ammonium chloride (50 mL). The organic layer was dried over anhydrous magnesium sulfate, filtered, and concentrated in vacuo. Purification of the residue by column chromatography, eluting with a gradient of 10 to 80% of ethyl acetate in heptane, provided the title compound as an orange oil.

The mixture of stereoisomers was subjected to:
Conditions A: chiral SFC (column: DAICEL CHIRALPAK IC (250 mm×30 mm, 10 μm), eluting with 20% of isopropanol containing 0.1% ammonium hydroxide in supercritical carbon dioxide, followed by,
Conditions B: chiral SFC (column: DAICEL CHIRALPAK OJ-H (250 mm×30 mm, 5 μm), eluting with 15% of ethanol containing 0.1% ammonium hydroxide in supercritical carbon dioxide, to afford the four stereoisomers:
P1 (retention time=3.894 min, Conditions A) as a colorless solid (0.077 g, 13% yield, 99% ee): ¹H-NMR (400 MHz; DMSO-d₆) δ 10.68-10.65 (m, 1H), 8.68-8.65 (m, 1H), 7.42-7.37 (m, 1H), 7.37-7.23 (m, 3H), 6.97 (s, 1H), 6.56-6.28 (m, 1H), 4.62-4.53 (m, 2H), 4.02-3.98 (m, 1H), 3.70-3.65 (m, 1H), 3.53-3.37 (m, 2H), 2.48-2.40 (m, 1H), 1.99-1.91 (m, 1H), 1.43-1.37 (m, 1H), 1.26-1.21 (m, 1H), 0.74-0.69 (m, 3H); MS (ES+) m/z 480.0 (M+1).
P2 (retention time=11.143 min, Conditions A) as a colorless solid (0.049 g, 8% yield, 99% ee): ¹H-NMR (400 MHz; DMSO-d₆) δ 10.71 (s, 1H), 8.67 (d, J=4.8 Hz, 1H), 7.36 (d, J=4.8 Hz, 1H), 7.35-7.22 (m, 3H), 6.98 (s, 1H), 6.42 (tt, J=54.0, 3.4 Hz, 1H), 4.58 (dt, J=14.8, 3.2 Hz, 2H), 3.97-3.89 (m, 1H), 3.83 (dd, J=11.2, 4.4 Hz, 1H), 3.01 (t, J=11.2 Hz, 1H), 2.84 (dt, J=10.8, 4.0 Hz, 1H), 2.45-2.38 (m, 1H), 2.24-2.10 (m, 1H), 1.83-1.61 (m, 2H), 0.49 (d, J=6.4 Hz, 3H); MS (ES+) m/z 480.0 (M+1).
P3 (retention time=0.630 min, Conditions B) as a colorless solid (0.027 g, 5% yield, 99% ee): ¹H-NMR (400 MHz; DMSO-d₆) δ 10.64 (d, J=1.2 Hz, 1H), 8.65 (d, J=4.8 Hz, 1H), 7.38 (d, J=5.2 Hz, 1H), 7.36-7.20 (m, 3H), 6.96 (s, 1H), 6.62-6.19 (m, 1H), 4.56 (dt, J=14.8, 2.8 Hz, 2H), 4.04-3.94 (m, 1H), 3.67 (d, J=11.2 Hz, 1H), 3.52-3.35 (m, 3H), 2.46-2.40 (m, 1H), 2.03-1.83 (m, 1H), 1.39 (d, J=12.0 Hz, 1H), 0.72 (d, J=7.2 Hz, 3H); MS (ES+) m/z 480.0 (M+1).
P4 (retention time=0.748 min, Conditions B) as a colorless solid (0.074 g, 13% yield, 99% ee): ¹H-NMR (400 MHz; DMSO-d₆) δ 10.72 (s, 1H), 8.68 (d, J=4.8 Hz, 1H), 7.36 (d, J=4.4 Hz, 1H), 7.35-7.21 (m, 3H), 6.98 (s, 1H), 6.62-6.11 (m, 1H), 4.58 (dt, J=14.8, 2.8 Hz, 2H), 3.97-3.89 (m, 1H), 3.83 (dd, J=11.2, 4.4 Hz, 1H), 3.01 (t, J=11.2 Hz, 1H), 2.90-2.76 (m, 1H), 2.46-2.41 (m, 1H), 2.23-2.13 (m, 1H), 1.83-1.63 (m, 2H), 0.49 (d, J=6.0 Hz, 3H); MS (ES+) m/z 480.0 (M+1).

### Example 343

### Synthesis of N-(4-(2,5-difluorophenyl)-2-(5,5-difluorotetrahydro-2H-pyran-2-yl)pyridin-3-yl)-1-(trifluoromethyl)-1H-pyrazole-4-carboxamide

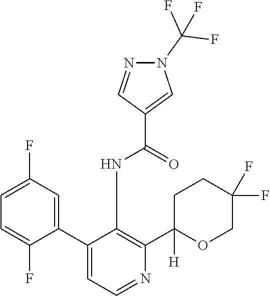 [SMILES: O=C(Nc1c(-c2cc(F)ccc2F)ccnc1C1CCC(F)(F)CO1)c1cnn(C(F)(F)F)c1]

### Step 1. Preparation of pure enantiomer (i.e., P1) 4-(2,5-difluorophenyl)-2-(5,5-difluorotetrahydro-2H-pyran-2-yl)pyridin-3-amine

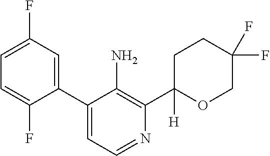 [SMILES: Nc1c(-c2cc(F)ccc2F)ccnc1C1CCC(F)(F)CO1]

A racemic mixture of 4-(2,5-difluorophenyl)-2-(5,5-difluorotetrahydro-2H-pyran-2-yl)pyridin-3-amine (2.7 g, 8.27 mmol) was subjected to chiral SFC purification (column: DAICEL CHIRALPAK AD (250 mm×30 mm, 10 μm), eluting with 20% of isopropanol containing 0.1% of ammonium hydroxide in supercritical carbon dioxide, to afford the 2 pure enantiomers of which P1 is used in further steps as is.

P1 (retention time=0.913 min) as a colorless solid (1.1 g, 41% yield, 99% ee): ¹H-NMR (400 MHz; DMSO-d₆) δ 7.87-7.83 (m, 1H), 7.44-7.30 (m, 2H), 7.28-7.22 (m, 1H), 7.04-6.99 (m, 1H), 5.03-4.89 (m, 2H), 4.84-4.76 (m, 1H), 4.01-3.85 (m, 2H), 2.44-2.27 (m, 2H), 2.26-2.08 (m, 1H), 2.04-1.91 (m, 1H); MS (ES+) m/z 327.2 (M+1).

### Step 2. Preparation of N-(4-(2,5-difluorophenyl)-2-(5,5-difluorotetrahydro-2H-pyran-2-yl)pyridin-3-yl)-1-(trifluoromethyl)-1H-pyrazole-4-carboxamide

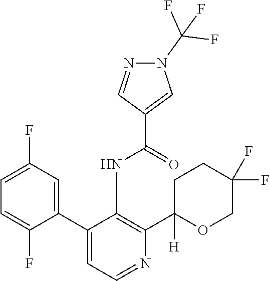 [SMILES: O=C(Nc1c(-c2cc(F)ccc2F)ccnc1C1CCC(F)(F)CO1)c1cnn(C(F)(F)F)c1]

To a mixture of P1-4-(2,5-difluorophenyl)-2-(5,5-difluorotetrahydro-2H-pyran-2-yl)pyridin-3-amine (0.080 g, 0.25 mmol), 1-(trifluoromethyl)-1H-pyrazole-4-carboxylic acid (0.066 g, 0.37 mmol) and 2-chloro-1-methylpyridinium iodide (0.16 g, 0.61 mmol) was added anhydrous tetrahydrofuran (4.9 mL). The solution was heated at 50° C. for 1 min before N-ethyl-N-isopropylpropan-2-amine (0.32 g, 0.43 mmol) was added. The reaction mixture was stirred at 50° C. for 96 h. The reaction mixture was cooled to ambient temperature and diluted with ethyl acetate (120 mL) and washed with 1M sodium hydroxide (50 mL), and saturated ammonium chloride solution (50 mL). The organic layer was dried over anhydrous magnesium sulfate, filtered, and concentrated in vacuo. The residue was purified by column chromatography, eluting with a gradient of 10 to 60% of ethyl acetate in heptane, to provide the title compound as a colorless solid (0.092 g, 74% yield): ¹H-NMR (400 MHz; DMSO-d₆) δ 10.03 (s, 1H), 9.01 (s, 1H), 8.66 (d, J=4.9 Hz, 1H), 8.32 (s, 1H), 7.51 (dd, J=4.9, 0.8 Hz, 1H), 7.37-7.23 (m, 3H), 4.89-4.87 (m, 1H), 3.96-3.90 (m, 1H), 3.82-3.71 (m, 1H), 2.34-2.13 (m, 3H), 1.96-1.92 (m, 1H); MS (ES+) m/z 489.2 (M+1).

### Example 344 and 345

### Synthesis of N-(4-(2,5-difluorophenyl)3-(trifluoromethyl)tetrahydrofuran-2-yl)pyridin-3-yl)-2-isopropylpyrimidine-5-carboxamide P1 and P2

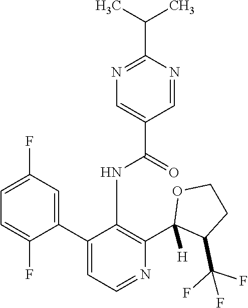 [SMILES: CC(C)c1ncc(C(=O)Nc2c(-c3cc(F)ccc3F)ccnc2[C@@H]2OCC[C@H]2C(F)(F)F)cn1]

### Step 1. Preparation of tert-butyl (E)-(2-(4-((tert-butyldimethylsilyl)oxy)but-1-en-1-yl)-4-(2,5-difluorophenyl)pyridin-3-yl)carbamate

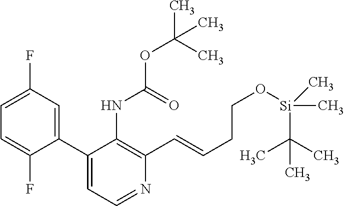 [SMILES: CC(C)(C)OC(=O)Nc1c(-c2cc(F)ccc2F)ccnc1/C=C/CCO[Si](C)(C)C(C)(C)C]

A mixture of tert-butyl (2-chloro-4-(2,5-difluorophenyl)pyridin-3-yl)carbamate (2.1 g, 6.2 mmol) in dioxane (31 mL) and water (3.4 mL) was degassed with nitrogen for 10 minutes. To the reaction mixture was added [1,1′-bis(diphenylphosphino)ferrocene]dichloropalladium(II) dichloromethane complex (0.52 g, 0.62 mmol), (E)-tert-butyldimethyl((4-(4,4,5,5-tetramethyl-1,3,2-dioxaborolan-2-yl)but-3-en-1-yl)oxy)silane (2.3 g, 7.4 mmol), and potassium carbonate (1.5 g, 11 mmol). The reaction mixture was stirred at 100° C. for 5 h. After cooling to ambient temperature, the reaction mixture was diluted in ethyl acetate (200 mL) and washed with saturated ammonium chloride (2×50 mL). The organic phase was dried over anhydrous magnesium sulfate, filtered, and concentrated in vacuo. The residue was purified by column chromatography, eluting with a gradient of 0 to 50% of ethyl acetate in heptane, to afford the title compound as a colorless oil (2.6 g, 88% yield): MS (ES+) m/z 491.6 (M+1).

### Step 2. Preparation of (E)-4-(3-amino-4-(2,5-difluorophenyl)pyridin-2-yl)but-3-en-1-ol

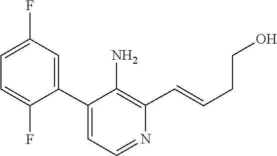 [SMILES: Nc1c(-c2cc(F)ccc2F)ccnc1/C=C/CCO]

A mixture of tert-butyl (E)-(2-(4-((tert-butyldimethylsilyl)oxy)but-1-en-1-yl)-4-(2,5-difluorophenyl)pyridin-3-yl)carbamate (2.6 g, 5.4 mmol) and 4M hydrochloric acid in 1,4-dioxane (27 mL) was stirred at ambient temperature for 18 h. The reaction mixture was diluted with ethyl acetate (500 mL) and 5M sodium hydroxide (40 mL). The layers were separated, and the organic layer was washed with brine (50 mL). The organic layer was dried over anhydrous magnesium sulfate, filtered, and concentrated in vacuo. Purification of the residue by column chromatography, eluting with 10 to 100% of ethyl acetate in heptane provided the title compound as an colorless solid (1.0 g, 67% yield): MS (ES+) m/z 277.2 (M+1).

### Step 3. Preparation of 4-(2,5-difluorophenyl)-2-(rac-(anti)-3-(trifluoromethyl)tetrahydrofuran-2-yl)pyridin-3-amine

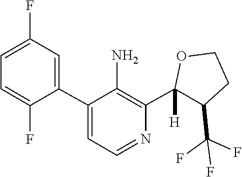 [SMILES: Nc1c(-c2cc(F)ccc2F)ccnc1[C@@H]1OCC[C@H]1C(F)(F)F]

To a mixture of (E)-4-(3-amino-4-(2,5-difluorophenyl)pyridin-2-yl)but-3-en-1-ol (0.50 g, 1.8 mmol), 5-(trifluoromethyl) dibenzothiophenium trifluoromethylsulfonate (1.2 g, 2.9 mmol) and tris(2-phenylpyridine)iridium (0.0059 g, 0.009 mmol) was added anhydrous dichloromethane (18 mL). The headspace of the vial was replaced with nitrogen, the vial was sealed, and the reaction mixture was stirred in front of Kessil PR160L lights (440 nm) for 18 h. The reaction mixture was diluted with ethyl acetate (120 mL) and washed with 1M sodium hydroxide (50 mL), and saturated ammonium chloride (50 mL). The organic layer was dried over anhydrous magnesium sulfate, filtered, and concentrated in vacuo. Purification of the residue by column chromatography, eluting with a gradient of 5 to 35% of ethyl acetate in heptane, provided the title compound as a colorless oil (0.40 g, 64% yield): ¹H-NMR (400 MHz; CDCl₃) δ 8.21 (d, J=5.5 Hz, 1H), 7.43-7.39 (m, 1H), 7.28-7.20 (m, 2H), 7.16 (ddd, J=8.1, 5.3, 2.9 Hz, 1H), 5.37 (d, J=4.6 Hz, 1H), 4.79-4.50 (m, 2H), 4.38-4.31 (m, 1H), 4.00 (q, J=8.0 Hz, 1H), 3.66-3.61 (m, 1H), 2.40 (dq, J=13.5, 8.8 Hz, 1H), 2.30 (dtt, J=10.3, 6.9, 3.4 Hz, 1H); MS (ES+) m/z 345.2 (M+1).

### Step 4. Preparation of N-(4-(2,5-difluorophenyl)-3-(trifluoromethyl)tetrahydrofuran-2-yl)pyridin-3-yl)-2-isopropylpyrimidine-5-carboxamide

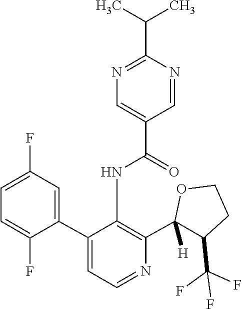 [SMILES: CC(C)c1ncc(C(=O)Nc2c(-c3cc(F)ccc3F)ccnc2[C@@H]2OCC[C@H]2C(F)(F)F)cn1]

To a mixture of 4-(2,5-difluorophenyl)-2-(rac-(anti)-3-(trifluoromethyl)tetrahydrofuran-2-yl)pyridin-3-amine (0.40 g, 1.2 mmol), 2-isopropylpyrimidine-5-carboxylic acid (0.25 g, 1.5 mmol) and 2-chloro-1-methylpyridinium iodide (0.77 g, 3.0 mmol) was added anhydrous tetrahydrofuran (23 mL). The solution was heated at 55° C. for 1 min before N-ethyl-N-isopropylpropan-2-amine (1.5 g, 12 mmol) was added. The reaction mixture was stirred at 55° C. for 18 h. The reaction mixture was diluted with methanol (10 mL), and 5M sodium hydroxide (4 mL), and heated to 45° C. for 1 h. The reaction mixture was cooled to ambient temperature and diluted with ethyl acetate (125 mL) and washed with 1M sodium hydroxide (50 mL), and saturated ammonium chloride solution (50 mL). The organic layer was dried over anhydrous magnesium sulfate, filtered, and concentrated in vacuo. The residue was purified by column chromatography, eluting with a gradient of 10 to 50% of ethyl acetate in heptane, to provide the mixture of enantiomers, which was subjected to chiral SFC (column: DAICEL CHIRALPAK ID (250 mm×30 mm, 10 μm), eluting with 20% of isopropanol in supercritical carbon dioxide, to provide the single enantiomers.

P1 (retention time=2.493 min) as a colorless solid (0.11 g, 19% yield, 99% ee): ¹H-NMR (400 MHz; DMSO-d₃) δ 10.57 (s, 1H), 8.96 (s, 2H), 8.70 (d, J=4.9 Hz, 1H), 7.58 (d, J=4.9 Hz, 1H), 7.40-7.35 (m, 1H), 7.30 (tt, J=8.2, 4.1 Hz, 2H), 5.48 (d, J=4.6 Hz, 1H), 4.00-3.85 (m, 3H), 3.20 (dt, J=13.8, 6.9 Hz, 1H), 2.43-2.38 (m, 1H), 2.14-2.09 (m, 1H), 1.28 (d, J=6.9 Hz, 6H); MS (ES+) m/z 493.2 (M+1).
P2 (retention time=2.876 min) as a colorless solid (0.10 g, 17% yield, 99% ee): ¹H-NMR (400 MHz; DMSO-d₆) δ 10.58 (s, 1H), 8.96 (d, J=3.1 Hz, 2H), 8.70 (d, J=4.9 Hz, 1H), 7.58-7.57 (m, 1H), 7.40-7.35 (m, 1H), 7.30 (qd, J=7.5, 4.0 Hz, 2H), 5.48 (d, J=4.6 Hz, 1H), 3.98 (t, J=7.2 Hz, 1H), 3.94-3.84 (m, 2H), 3.20 (quintet, J=6.9 Hz, 1H), 2.44-2.38 (m, 1H), 2.15-2.09 (m, 1H), 1.28 (d, J=6.9 Hz, 6H); MS (ES+) m/z 493.2 (M+1).

### Example 346 and 347

### Synthesis of N-(4-(2,5-difluorophenyl)-2-3-(trifluoromethyl)tetrahydro-2H-pyran-4-yl)pyridin-3-yl)-2-isopropylpyrimidine-5-carboxamide P1 and P2

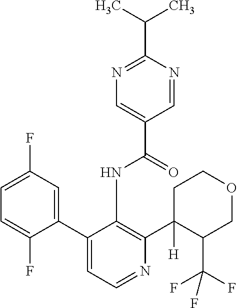 [SMILES: CC(C)c1ncc(C(=O)Nc2c(-c3cc(F)ccc3F)ccnc2C2CCOCC2C(F)(F)F)cn1]

### Step 1. Preparation of tert-butyl (4-(2,5-difluorophenyl)-2-(3,6-dihydro-2H-pyran-4-yl)pyridin-3-yl)carbamate

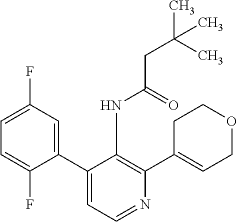 [SMILES: CC(C)(C)CC(=O)Nc1c(-c2cc(F)ccc2F)ccnc1C1=CCOCC1]

A mixture of tert-butyl (2-chloro-4-(2,5-difluorophenyl)pyridin-3-yl)carbamate (2.0 g, 5.9 mmol) in dioxane (29 mL) and water (3.3 mL) was degassed with nitrogen for 10 minutes. To the reaction mixture was added [1,1′-bis(diphenylphosphino)ferrocene]dichloropalladium(II) dichloromethane complex (0.50 g, 0.59 mmol), 3,6-dihydro-2H-pyran-4-boronic acid pinacol ester (1.6 g, 7.6 mmol), and potassium carbonate (2.0 g, 15 mmol). The reaction mixture was stirred at 85° C. for 18 h. After cooling to ambient temperature, the reaction mixture was diluted with ethyl acetate (600 mL) and filtered through Celite. The organic layer was washed with saturated ammonium chloride (200 mL), dried over anhydrous magnesium sulfate, filtered, and concentrated in vacuo. The residue was purified by column chromatography, eluting with a gradient of 5 to 35% of ethyl acetate in heptane, to afford the title compound as a colorless solid (1.5 g, 66% yield): MS (ES+) m/z 389.4 (M+1).

### Step 2. Preparation of 4-(2,5-difluorophenyl)-2-(3,6-dihydro-2H-pyran-4-yl)pyridin-3-amine

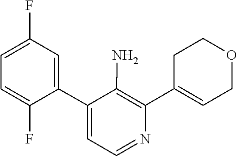 [SMILES: Nc1c(-c2cc(F)ccc2F)ccnc1C1=CCOCC1]

A mixture of tert-butyl (4-(2,5-difluorophenyl)-2-(3,6-dihydro-2H-pyran-4-yl)pyridin-3-yl)carbamate (0.82 g, 2.1 mmol) and 4M hydrochloric acid in 1,4-dioxane (11 mL) was stirred at 40° C. for 1 h. The reaction mixture was diluted with ethyl acetate (300 mL) and 5M sodium hydroxide (15 mL). The layers were separated, and the organic layer was washed with 1M sodium hydroxide (50 mL) and saturated ammonium chloride (50 mL). The organic layer was dried over anhydrous magnesium sulfate, filtered, and concentrated in vacuo. Purification of the residue by column chromatography, eluting with a gradient of 5 to 60% of ethyl acetate in heptane provided the title compound as an colorless solid (0.50 g, 83% yield): MS (ES+) m/z 289.2 (M+1).

### Step 3. Preparation of 4-(2,5-difluorophenyl)-2-(rac-(anti)-3-(trifluoromethyl)tetrahydro-2H-pyran-4-yl)pyridin-3-amine

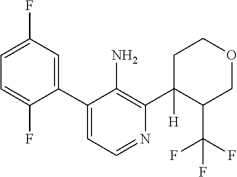 [SMILES: Nc1c(-c2cc(F)ccc2F)ccnc1C1CCOCC1C(F)(F)F]

A mixture of 4-(2,5-difluorophenyl)-2-(3,6-dihydro-2H-pyran-4-yl)pyridin-3-amine (0.61 g, 2.1 mmol), and anhydrous dimethylsulfoxide (21 mL) was sparged with nitrogen for 5 min. To the mixture was added trifluoroacetic acid (0.17 mL), 5-(trifluoromethyl) dibenzothiophenium trifluoromethylsulfonate (1.3 g, 3.1 mmol) and tris(2,2′-bipyridyl)dichlororuthenium(II) hexahydrate (0.034 g, 0.053 mmol). The vial was sealed, and the reaction mixture was stirred in front of Kessil PR160L lights (440 nm) for 18 h. The reaction mixture was diluted with ethyl acetate (120 mL) and washed with saturated ammonium chloride (50 mL). The organic layer was dried over anhydrous magnesium sulfate, filtered, and concentrated in vacuo. The residue was subjected to column chromatography, eluting with a gradient of 5 to 60% of ethyl acetate in heptane. The mixture obtained was dissolved in ethanol (22 mL), and acetic acid (0.5 mL) and sparged with nitrogen for 10 min. A mixture of 10% palladium hydroxide on carbon (0.34 g) and ammonium formate (1.4 g, 22 mmol) was added and the reaction mixture was heated to reflux for 1 h. A mixture of 10% palladium hydroxide on carbon (0.34 g) and ammonium formate (1.4 g, 22 mmol) was added and the reaction was heated to reflux for a further 5 h. The reaction mixture was cooled to ambient temperature and filtered through a bed of Celite, washing the residue with ethyl acetate (5×20 mL). The organic solution was concentrated in vacuo. Purification of the residue by column chromatography, eluting with a gradient of 5 to 60% of ethyl acetate in heptane, provided the title compound as a colorless oil (0.11 g, 14% yield): MS (ES+) m/z 359.4 (M+1).

### Step 4. Preparation of N-(4-(2,5-difluorophenyl)-2-3-(trifluoromethyl)tetrahydro-2H-pyran-4-yl)pyridin-3-yl)-2-isopropylpyrimidine-5-carboxamide P1 and P2

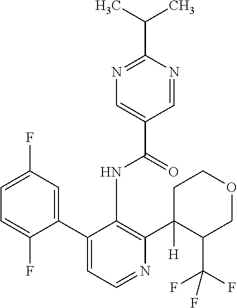 [SMILES: CC(C)c1ncc(C(=O)Nc2c(-c3cc(F)ccc3F)ccnc2C2CCOCC2C(F)(F)F)cn1]

To a mixture of 4-(2,5-difluorophenyl)-2-(rac-(3R,4R)-3-(trifluoromethyl)tetrahydro-2H-pyran-4-yl)pyridin-3-amine (0.11 g, 0.30 mmol), 2-isopropylpyrimidine-5-carboxylic acid (0.099 g, 0.60 mmol) and 2-chloro-1-methylpyridinium iodide (0.31 g, 1.2 mmol) was added anhydrous tetrahydrofuran (6.0 mL). The solution was heated at 65° C. for 1 min before N-ethyl-N-isopropylpropan-2-amine (0.39 g, 3.0 mmol) was added. The reaction mixture was stirred at 65° C. for 18 h. The reaction mixture was cooled to ambient temperature and diluted with ethyl acetate (100 mL) and washed with 1M sodium hydroxide (50 mL), and saturated ammonium chloride solution (50 mL). The organic layer was dried over anhydrous magnesium sulfate, filtered, and concentrated in vacuo. The residue was purified by column chromatography, eluting with a gradient of 10 to 100% of ethyl acetate in heptane, to provide the mixture of enantiomers which was subjected to chiral SFC (column: Lux Cel-2 (250 mm×10 mm, 10 μm), eluting with 8% of isopropanol in supercritical carbon dioxide, to provide the single enantiomers.

P1 (retention time=3.148 min) as a colorless solid (0.011 g, 4% yield, 99% ee): ¹H-NMR (400 MHz; DMSO-d₆) δ 10.34 (d, J=0.6 Hz, 1H), 8.94 (d, J=3.2 Hz, 2H), 8.66 (d, J=4.9 Hz, 1H), 7.44 (d, J=4.9 Hz, 1H), 7.39-7.34 (m, 1H), 7.30-7.23 (m, 2H), 4.21-4.18 (m, 1H), 4.11-4.08 (m, 1H), 3.96-3.69 (bs, 1H), 3.69-3.66 (m, 1H), 3.59-3.53 (m, 1H), 3.20 (dt, J=13.8, 6.9 Hz, 1H), 3.11-2.80 (bs, 1H), 2.65-2.62 (m, 1H), 1.61-1.57 (m, 1H), 1.29 (d, J=6.9 Hz, 6H); MS (ES+) m/z 507.2 (M+1).
P2 (retention time=3.222 min) as a colorless solid (0.0093 g, 3% yield, 98% ee): ¹H-NMR (400 MHz; DMSO-d₆) δ 10.35-10.32 (m, 1H), 8.95-8.93 (m, 2H), 8.67-8.66 (m, 1H), 7.44-7.43 (m, 1H), 7.40-7.33 (m, 1H), 7.32-7.27 (m, 1H), 7.27-7.21 (m, 1H), 4.21-4.17 (m, 1H), 4.11-4.07 (m, 1H), 3.96-3.69 (bs, 1H), 3.70-3.65 (m, 1H), 3.60-3.53 (m, 1H), 3.20 (dt, J=13.8, 6.9 Hz, 1H), 3.11-2.80 (bs, 1H), 2.68-2.61 (m, 1H), 1.61-1.56 (m, 1H), 1.28 (t, J=6.6 Hz, 6H); MS (ES+) m/z 507.2 (M+1).

### Example 348

### Synthesis of N-(4-(2,5-difluorophenyl)-2-(rac-anti-2-(trifluoromethyl)tetrahydrofuran-3-yl)pyridin-3-yl)-2-isopropylpyrimidine-5-carboxamide

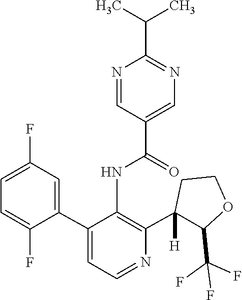 [SMILES: CC(C)c1ncc(C(=O)Nc2c(-c3cc(F)ccc3F)ccnc2[C@@H]2CCO[C@H]2C(F)(F)F)cn1]

### Step 1. Preparation of 4-(2,5-difluorophenyl)-2-(rac-(anti)-2-(trifluoromethyl)tetrahydrofuran-3-yl) pyridin-3-amine

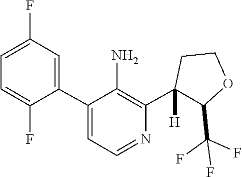 [SMILES: Nc1c(-c2cc(F)ccc2F)ccnc1[C@@H]1CCO[C@H]1C(F)(F)F]

A mixture of tert-butyl (2-chloro-4-(2,5-difluorophenyl)pyridin-3-yl)carbamate (0.55 g, 1.6 mmol), 2-trifluoromethyl-tetrahydro-furan-3-carboxylic acid (0.48 g, 2.6 mmol), (4,4″-di-t-butyl-2,2″-bipyridine)bis[3,5-difluoro-2-[5-trifluoromethyl-2-pyridinyl-kN)phenyl-kC]iridium(II) hexafluorophosphate (0.018 g, 0.016 mmol), dichloro(dimethoxyethane)nickel (0.035 g, 0.16 mmol), 4-tert-butyl-2-(4-tert-butyl-2-pyridyl)pyridine (0.065 g, 0.24 mmol), cesium carbonate (0.92 g, 2.8 mmol), and N,N-dimethylformamide (40 mL). The headspace of the vial was replaced with nitrogen, the vial was sealed, and the reaction mixture was stirred in front of Kessil PR160L lights (440 nm) for 60 h. The reaction mixture was diluted with ethyl acetate (300 mL) and washed with 1M sodium hydroxide (50 mL), water (5×50 mL), and brine (50 mL). The organic layer was dried over anhydrous magnesium sulfate, filtered, and concentrated in vacuo. The residue was dissolved in 4M hydrogen chloride in 1,4-dioxane (8.1 mL) and stirred at ambient temperature for 3 h. The reaction mixture was diluted with ethyl acetate (200 mL) and 5M sodium hydroxide (15 mL). The layers were separated, and the organic layer was washed with saturated ammonium chloride (25 mL), dried over anhydrous magnesium sulfate, filtered, and concentrated in vacuo. The residue was purified by column chromatography, eluting with a gradient of 5 to 80% of ethyl acetate in heptane, to provide the title compound as a colorless solid (0.40 g, 71% yield): MS (ES+) m/z 345.4 (M+1).

### Step 2. Preparation of N-(4-(2,5-difluorophenyl)-2-(rac-(anti)-2-(trifluoromethyl)tetrahydrofuran-3-yl)pyridin-3-yl)-2-isopropylpyrimidine-5-carboxamide

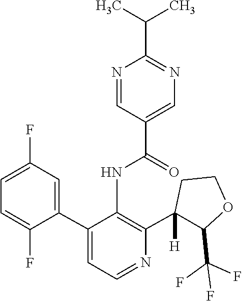 [SMILES: CC(C)c1ncc(C(=O)Nc2c(-c3cc(F)ccc3F)ccnc2[C@@H]2CCO[C@H]2C(F)(F)F)cn1]

To a mixture of 4-(2,5-difluorophenyl)-2-(rac-(2R,3S)-2-(trifluoromethyl)tetrahydrofuran-3-yl)pyridin-3-amine (0.40 g, 1.1 mmol), 2-isopropylpyrimidine-5-carboxylic acid (0.27 g, 1.6 mmol) and 2-chloro-1-methylpyridinium iodide (0.82 g, 3.2 mmol) was added anhydrous tetrahydrofuran (23 mL). The solution was heated at 65° C. for 1 min before N-ethyl-N-isopropylpropan-2-amine (1.5 g, 11 mmol) was added. The reaction mixture was stirred at 65° C. for 18 h. The reaction mixture was diluted with methanol (5 mL) and 5M sodium hydroxide (5 mL), and heated to 40° C. for 20 min. The reaction mixture was cooled to ambient temperature and diluted with ethyl acetate (120 mL) and washed with 1M sodium hydroxide (50 mL), and saturated ammonium chloride solution (50 mL). The organic layer was dried over anhydrous magnesium sulfate, filtered, and concentrated in vacuo. The residue was purified by column chromatography eluting with a gradient of 10 to 50% of ethyl acetate in heptane to provide the title compound as a colorless solid (0.25 g, 43% yield): ¹H-NMR (400 MHz; DMSO-d₆) δ 10.52 (s, 1H), 8.97-8.94 (m, 2H), 8.68 (d, J=4.9 Hz, 1H), 7.50 (d, J=4.9 Hz, 1H), 7.41-7.35 (m, 1H), 7.30 (td, J=7.6, 4.3 Hz, 2H), 5.09-4.70 (bs, 1H), 4.15-4.07 (m, 2H), 4.02-3.95 (m, 1H), 3.20 (quintet, J=6.9 Hz, 1H), 2.49-2.41 (m, 1H), 2.14-2.07 (m, 1H), 1.26 (s, 6H); MS (ES+) m/z 493.2 (M+1).

### Example 349 and 350

### Synthesis of N-(4-(2,5-difluorophenyl)-2-(3-(trifluoromethyl)tetrahydro-2H-pyran-2-yl)pyridin-3-yl)-2-isopropylpyrimidine-5-carboxamide P1, D1 and P2, D1

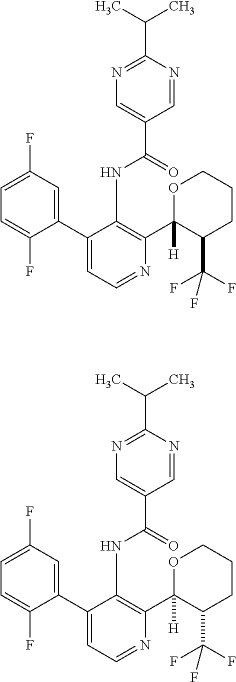 [SMILES: CC(C)c1ncc(C(=O)Nc2c(-c3cc(F)ccc3F)ccnc2[C@@H]2OCCC[C@H]2C(F)(F)F)cn1 ; CC(C)c1ncc(C(=O)Nc2c(-c3cc(F)ccc3F)ccnc2[C@H]2OCCC[C@@H]2C(F)(F)F)cn1]

### Step 1. Preparation of tert-butyl (2-(4-((tert-butyldimethylsilyl)oxy)but-1-en-1-yl)-4-(2,5-difluorophenyl)pyridin-3-yl)carbamate

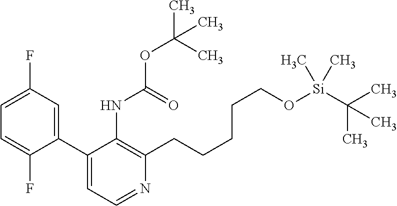 [SMILES: CC(C)(C)OC(=O)Nc1c(-c2cc(F)ccc2F)ccnc1CCCCCO[Si](C)(C)C(C)(C)C]

A mixture of tert-butyl (2-chloro-4-(2,5-difluorophenyl)pyridin-3-yl)carbamate (1.1 g, 3.2 mmol) in dioxane (16 mL) and water (1.8 mL) was degassed with nitrogen for 10 minutes. To the reaction mixture was added [1,1′-bis(diphenylphosphino)ferrocene]dichloropalladium(II) dichloromethane complex (0.27 g, 0.32 mmol), tert-butyl-dimethyl-[(E)-5-(4,4,5,5-tetramethyl-1,3,2-dioxaborolan-2-yl)pent-4-enoxy]silane (1.3 g, 3.9 mmol), and potassium carbonate (0.80 g, 5.8 mmol). The reaction was stirred at 100° C. for 5 h. After cooling to ambient temperature, the reaction mixture was diluted in ethyl acetate (200 mL) and washed with saturated ammonium chloride (2×50 mL). The organic phase was dried over anhydrous magnesium sulfate, filtered, and concentrated in vacuo. The residue was purified by column chromatography, eluting with a gradient of 0 to 50% of ethyl acetate in heptane, to afford the title compound as a mixture of cis and trans isomers used in the next step as is (1.6 g, 100% yield): MS (ES+) m/z 505.6 (M+1).

### Step 2. Preparation of (E)-5-(3-amino-4-(2,5-difluorophenyl)pyridin-2-yl)pent-4-en-1-ol

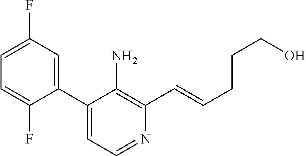 [SMILES: Nc1c(-c2cc(F)ccc2F)ccnc1/C=C/CCCO]

A mixture of tert-butyl (2-(4-((tert-butyldimethylsilyl)oxy)but-1-en-1-yl)-4-(2,5-difluorophenyl)pyridin-3-yl)carbamate (1.6 g, 3.3 mmol) and 4M hydrochloric acid in 1,4-dioxane (16 mL) was stirred at ambient temperature for 90 min. The reaction mixture was diluted with ethyl acetate (250 mL) and 5M sodium hydroxide (25 mL). The layers were separated, and the organic layer was washed with brine (50 mL). The organic layer was dried over anhydrous magnesium sulfate, filtered, and concentrated in vacuo. Purification of the residue by column chromatography, eluting with a gradient of 50 to 100% of ethyl acetate in heptane, provided the title compound as an colorless solid (0.68 g, 72% yield): ¹H-NMR (400 MHz; MeOD-d₄) δ 7.88 (t, J=4.9 Hz, 1H), 7.33-7.15 (m, 3H), 6.97 (d, J=4.9 Hz, 1H), 6.75-6.63 (m, 2H), 3.66 (t, J=6.5 Hz, 2H), 2.42 (q, J=7.1 Hz, 2H), 1.79 (quintet, J=7.2 Hz, 2H); MS (ES+) m/z 291.2 (M+1).

### Step 3. Preparation of 4-(2,5-difluorophenyl)-2-(rac-(2R,3R)-3-(trifluoromethyl)tetrahydro-2H-pyran-2-yl)pyridin-3-amine

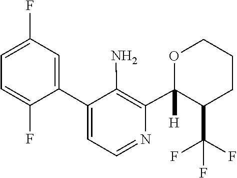

To a mixture of (E)-5-(3-amino-4-(2,5-difluorophenyl)pyridin-2-yl)pent-4-en-1-ol (0.15 g, 0.52 mmol), 5-(trifluoromethyl) dibenzothiophenium trifluoromethylsulfonate (0.25 g, 0.62 mmol) and tris(2-phenylpyridine)iridium (0.0017 g, 0.0026 mmol) was added anhydrous dichloromethane (3.2 mL). The headspace of the vial was replaced with nitrogen, the vial was sealed, and the reaction mixture was stirred in front of Kessil PR160L lights (440 nm) for 90 min. The reaction mixture was concentrated in vacuo. Purification of the residue by column chromatography, eluting with a gradient of 0 to 100% of ethyl acetate in heptane, provided the title compound as a colorless oil (0.18 g, 99% yield): ¹H-NMR (400 MHz; CDCl₃) δ 8.02 (d, J=4.8 Hz, 1H), 7.17 (td, J=8.8, 4.5 Hz, 1H), 7.13-7.04 (m, 2H), 6.99 (d, J=4.8 Hz, 1H), 4.93 (d, J=7.5 Hz, 1H), 4.41 (s, 2H), 3.93-3.88 (m, 1H), 3.64 (ddd, J=11.7, 9.3, 2.7 Hz, 1H), 3.34-3.31 (m, 1H), 2.32-2.27 (m, 1H), 1.86 (tdd, J=11.2, 7.6, 3.5 Hz, 2H), 1.69 (dt, J=8.8, 4.3 Hz, 1H); MS (ES+) m/z 359.2 (M+1).

### Step 4. Preparation of N-(4-(2,5-difluorophenyl)-2-(3-(trifluoromethyl)tetrahydro-2H-pyran-2-yl)pyridin-3-yl)-2-isopropylpyrimidine-5-carboxamide and N-(4-(2,5-difluorophenyl)-2-((2S,3S)-3-(trifluoromethyl)tetrahydro-2H-pyran-2-yl)pyridin-3-yl)-2-isopropylpyrimidine-5-carboxamide

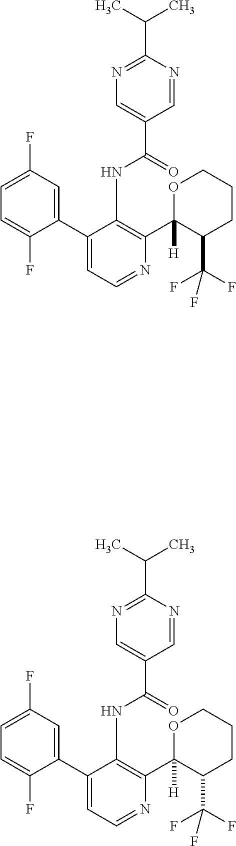 [SMILES: CC(C)c1ncc(C(=O)Nc2c(-c3cc(F)ccc3F)ccnc2[C@@H]2OCCC[C@H]2C(F)(F)F)cn1 ; CC(C)c1ncc(C(=O)Nc2c(-c3cc(F)ccc3F)ccnc2[C@H]2OCCC[C@@H]2C(F)(F)F)cn1]

To a mixture of 4-(2,5-difluorophenyl)-2-(rac-(2R,3R)-3-(trifluoromethyl)tetrahydro-2H-pyran-2-yl)pyridin-3-amine (0.18 g, 0.52 mmol), 2-isopropylpyrimidine-5-carboxylic acid (0.14 g, 0.86 mmol) and 2-chloro-1-methylpyridinium iodide (0.34 g, 1.3 mmol) was added anhydrous tetrahydrofuran (11 mL). The solution was heated at 55° C. for 1 min before N-ethyl-N-isopropylpropan-2-amine (0.74 g, 5.7 mmol) was added. The reaction mixture was stirred at 55° C. for 18 h. The reaction mixture was diluted with methanol (5 mL), and 5M sodium hydroxide (3 mL), and heated to 45° C. for 1 h. The reaction mixture was cooled to ambient temperature and diluted with ethyl acetate (150 mL) and washed with 1M sodium hydroxide (50 mL), and saturated ammonium chloride solution (50 mL). The organic layer was dried over anhydrous magnesium sulfate, filtered, and concentrated in vacuo. The residue was purified by column chromatography, eluting with a gradient of 10 to 60% of ethyl acetate in heptane, to provide the mixture of enantiomers which was subjected to chiral SFC (column: DAICEL CHIRALPAK IC (250 mm×30 mm, 10 μm), eluting with 45% of a 1:1 mixture of acetonitrile:ethanol with 10 mM ammonium formate in supercritical carbon dioxide, to provide the single enantiomers.

P1 (retention time=2.344 min) as a colorless solid (0.028 g, 10% yield, 99% ee): ¹H-NMR (400 MHz; DMSO-d₆) δ 10.51 (s, 1H), 8.95 (s, 2H), 8.69-8.68 (m, 1H), 7.52 (d, J=4.8 Hz, 1H), 7.39-7.24 (m, 3H), 4.88 (d, J=9.4 Hz, 1H), 3.92-3.89 (m, 1H), 3.55-3.49 (m, 1H), 3.25-3.16 (m, 2H), 2.15-2.12 (m, 1H), 1.74-1.63 (m, 3H), 1.30-1.27 (m, 6H); MS (ES+) m/z 507.2 (M+1).
P2 (retention time=2.620 min) as a colorless solid (0.025 g, 9% yield, 99% ee): ¹H-NMR (400 MHz; DMSO-d₆) δ 10.50 (d, J=6.3 Hz, 1H), 8.96 (d, J=4.8 Hz, 2H), 8.68 (d, J=4.9 Hz, 1H), 7.53-7.51 (m, 1H), 7.39-7.24 (m, 3H), 4.88 (d, J=9.4 Hz, 1H), 3.92-3.89 (m, 1H), 3.55-3.49 (m, 1H), 3.26-3.16 (m, 2H), 2.16-2.12 (m, 1H), 1.74-1.62 (m, 3H), 1.30-1.26 (m, 6H); MS (ES+) m/z 507.2 (M+1).

### Example 351

### Synthesis of N-(3-(4,4-difluorocyclohexyl)-5-(2,5-difluorophenyl)pyridazin-4-yl)-3-(2,2-difluoroethoxy)isoxazole-5-carboxamide

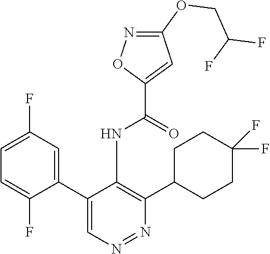 [SMILES: O=C(Nc1c(-c2cc(F)ccc2F)cnnc1C1CCC(F)(F)CC1)c1cc(OCC(F)F)no1]

### Step 1. Preparation of 5-chloro-3-(4,4-difluorocyclohex-1-en-1-yl)pyridazin-4-amine

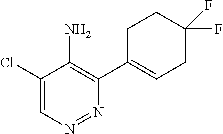 [SMILES: Nc1c(Cl)cnnc1C1=CCC(F)(F)CC1]

To a mixture of 3,5-dichloropyridazin-4-amine (0.50 g, 3.0 mmol) in 1,2-dimethoxyethane (12.5 mL) and a saturated potassium carbonate solution (6.2 mL) was added tetrakis(triphenylphosphine)palladium (0.21 g, 0.18 mmol), and 2-(4,4-difluorocyclohex-1-en-1-yl)-4,4,5,5-tetramethyl-1,3,2-dioxaborolane (1.1 g, 3.3 mmol). The resulting mixture was sealed under a nitrogen atmosphere and heated to 120° C. for 90 min under microwave irradiation. After cooling to ambient temperature, the reaction mixture was diluted in ethyl acetate (100 mL) and washed with saturated ammonium chloride (2×50 mL). The organic layer was dried over anhydrous magnesium sulfate, filtered, and concentrated in vacuo. The residue was purified by column chromatography, eluting with a gradient of 5 to 60% of ethyl acetate in heptane, to afford the title compound as a pink solid (0.33 g, 44% yield): MS (ES+) m/z 246.2 (M+1), 248.2 (M+1).

### Step 2. Preparation of 3-(4,4-difluorocyclohex-1-en-1-yl)-5-(2,5-difluorophenyl)pyridazin-4-amine

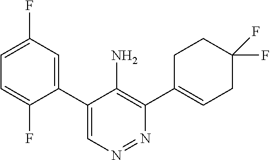 [SMILES: Nc1c(-c2cc(F)ccc2F)cnnc1C1=CCC(F)(F)CC1]

A mixture of 5-chloro-3-(4,4-difluorocyclohex-1-en-1-yl)pyridazin-4-amine (0.33 g, 1.4 mmol) in 1,4-dioxane (6.8 mL) and water (0.75 mL) was degassed with nitrogen for 10 minutes. To the reaction mixture was added [1,1′-bis(diphenylphosphino)ferrocene]dichloropalladium(II) dichloromethane complex (0.11 g, 0.14 mmol), 2,5-difluorophenylboronic acid (0.32 g, 2.0 mmol), and potassium carbonate (0.56 g, 4.0 mmol). The reaction mixture was stirred at 85° C. for 2 h. After cooling to ambient temperature, the reaction mixture was diluted with ethyl acetate (100 mL) and washed with saturated ammonium chloride (2×50 mL). The organic phase was dried over anhydrous magnesium sulfate, filtered, and concentrated in vacuo. The residue was purified by column chromatography, eluting with a gradient of 15 to 75% of ethyl acetate in heptane, to afford the title compound as a mixture with the starting material used as is in the next reaction (0.38 g): MS (ES+) m/z 324.2 (M+1).

### Step 3. Preparation of 3-(4,4-difluorocyclohexyl)-5-(2,5-difluorophenyl)pyridazin-4-amine

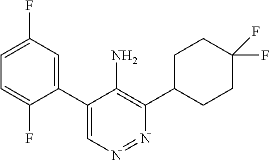 [SMILES: Nc1c(-c2cc(F)ccc2F)cnnc1C1CCC(F)(F)CC1]

A mixture of 3-(4,4-difluorocyclohex-1-en-1-yl)-5-(2,5-difluorophenyl)pyridazin-4-amine (0.38 g, 1.2 mmol), ethyl acetate (6.0 mL), methanol (6.0 mL), and acetic acid (0.2 mL) was sparged with nitrogen for 10 min before 10% palladium on carbon (0.13 g) was added. The solution was sparged with hydrogen for 10 min and left at ambient temperature under a hydrogen atmosphere for 18 h. The reaction mixture was diluted with ethyl acetate (50 mL), and filtered through a bed of Celite, washing the residue with ethyl acetate (3×25 mL). The filtrate was concentrated in vacuo. Purification of the residue by column chromatography, eluting with a gradient of 5 to 50% of ethyl acetate in heptane, provided the title compound as a colorless solid (0.14 g, 36% yield): MS (ES+) m/z 326.2 (M+1).

### Step 4. Preparation of N-(3-(4,4-difluorocyclohexyl)-5-(2,5-difluorophenyl)pyridazin-4-yl)-3-(2,2-difluoroethoxy)isoxazole-5-carboxamide

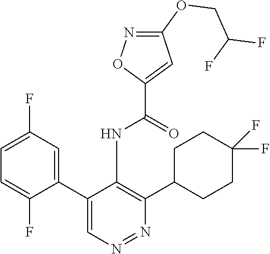 [SMILES: O=C(Nc1c(-c2cc(F)ccc2F)cnnc1C1CCC(F)(F)CC1)c1cc(OCC(F)F)no1]

To a mixture of 3-(4,4-difluorocyclohexyl)-5-(2,5-difluorophenyl)pyridazin-4-amine (0.14 g, 0.43 mmol), 3-(2,2-difluoroethoxy)isoxazole-5-carboxylic acid (0.17 g, 0.85 mmol) and 2-chloro-1-methylpyridinium iodide (0.27 g, 1.1 mmol) was added anhydrous tetrahydrofuran (8.5 mL). The solution was heated at 50° C. for 1 min before N-ethyl-N-isopropylpropan-2-amine (0.55 g, 4.3 mmol) was added. The reaction mixture was stirred at 50° C. for 60 h. The reaction mixture was cooled to ambient temperature and diluted with ethyl acetate (120 mL) and washed with 1M sodium hydroxide (50 mL), and saturated ammonium chloride solution (50 mL). The organic layer was dried over anhydrous magnesium sulfate, filtered, and concentrated in vacuo. The residue was purified by column chromatography, eluting with a gradient of 10 to 90% of ethyl acetate in heptane, followed by reverse phase column chromatography, eluting with a gradient of 30 to 90% acetonitrile in water, to provide the title compound as a colorless solid (0.049 g, 22% yield): ¹H-NMR (400 MHz; DMSO-d₆) δ 11.08-11.05 (m, 1H), 9.25 (s, 1H), 7.44-7.35 (m, 3H), 7.07 (d, J=4.8 Hz, 1H), 6.42 (tt, J=53.8, 3.1 Hz, 1H), 4.63-4.55 (m, 2H), 3.33-3.26 (m, 1H), 2.20-2.12 (m, 2H), 2.06-1.88 (m, 6H); MS (ES+) m/z 501.2 (M+1).

### Example 352

### Synthesis of tert-butyl N-[2-(4,4-difluorocyclohexyl)-4-(2,5-difluorophenyl)-3-pyridyl]carbamate

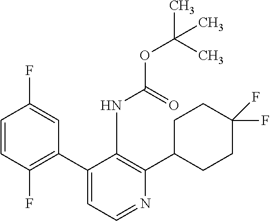 [SMILES: CC(C)(C)OC(=O)Nc1c(-c2cc(F)ccc2F)ccnc1C1CCC(F)(F)CC1]

To tert-butyl N-[2-chloro-4-(2,5-difluorophenyl)-3-pyridyl]carbamate (1.5 g, 4.6 mmol) was added 4,4-difluorocyclohexanecarboxylic acid (1.3 g, 7.9 mmol), (4,4″-di-t-butyl-2,2″-bipyridine)bis[3,5-difluoro-2-[5-trifluoromethyl-2-pyridinyl-kN)phenyl-kC]iridium(III) hexafluorophosphate (0.052 g, 0.046 mmol), dichloro(dimethoxyethane)nickel (0.10 g, 0.46 mmol), 4-tert-butyl-2-(4-tert-butyl-2-pyridyl)pyridine (0.19 g, 0.70 mmol), cesium carbonate (2.7 g, 8.4 mmol), and N,N-dimethylformamide (77 mL). The headspace of the vial was replaced with nitrogen, the vial was sealed, and the reaction mixture was stirred in front of Kessil PR160L lights (440 nm) for 24 h. The reaction mixture was diluted with ethyl acetate (250 mL) and washed with saturated ammonium chloride solution (2×50 mL), water (50 mL), and brine (50 mL). The organic layer was dried over anhydrous magnesium sulfate, filtered, and concentrated in vacuo. Purification of the residue by column chromatography, eluting with 0 to 40% of ethyl acetate in heptane, afforded the title compound as a yellow solid (0.98 g, 52% yield): ¹H-NMR (400 MHz; DMSO-d₆) δ 8.76 (s, 1H), 8.52 (d, J=4.9 Hz, 1H), 7.41-7.30 (m, 2H), 7.30-7.27 (m, 1H), 7.19-7.15 (m, 1H), 3.17-3.11 (m, 1H), 2.18-2.10 (m, 2H), 1.98-1.79 (m, 6H), 1.28-1.08 (m, 9H); MS (ES+) m/z 425.2 (M+1).

### Example 353-376

Single enantiomers of the given examples were obtained by chiral SFC using the conditions specified:

| | Structure | | |
|---|---|---|---|
| Example | Name | SFC | Retention time (Rₜ), ee; ¹H |
| No. | P1 or P2 | Conditions | NMR; MS (ES+) m/z |
| 353 | 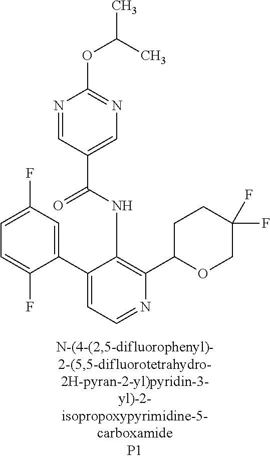 [SMILES: CC(C)Oc1ncc(C(=O)Nc2c(-c3cc(F)ccc3F)ccnc2C2CCC(F)(F)CO2)cn1] | Chiralpak AD Column (4.6 × 50 mm, 3 μm) eluting with 25% isopropanol containing 0.1% ammonium hydroxide in supercritical carbon dioxide | 1.086 min, 99% ee; (400 MHz, DMSO-d₆) δ 10.21 (s, 1H), 8.86 (s, 2H), 8.66 (d, J = 4.8 Hz, 1H), 7.51 (d, J = 4.4 Hz, 1H), 7.34 (td, J = 9.2, 4.4 Hz, 1H), 7.27 (dt, J = 8.0, 4.4 Hz, 2H), 5.26 (td, J = 12.4, 6.0 Hz, 1H), 4.90 (d, J = 9.2 Hz, 1H), 3.95- 3.87 (m, 1H), 3.77 (td, J = 19.2, 12.0 Hz, 1H), 2.31-2.13 (m, 3H), 2.00-1.89 (m, 1H), 1.33 (d, J = 6.0 Hz, 6H); MS (ES+) m/z 491.2 (M + 1) |
| 354 | 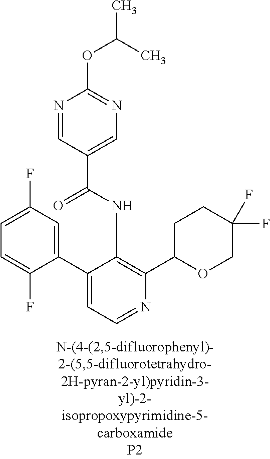 [SMILES: CC(C)Oc1ncc(C(=O)Nc2c(-c3cc(F)ccc3F)ccnc2C2CCC(F)(F)CO2)cn1] | Chiralpak AD Column (4.6 × 50 mm, 3 μm) eluting with 25% isopropanol containing 0.1% ammonium hydroxide in supercritical carbon dioxide | 1.188 min, 99% ee; (400 MHz, DMSO-d₆) δ 10.21 (s, 1H), 8.86 (s, 2H), 8.66 (d, J = 4.8 Hz, 1H), 7.51 (d, J = 4.8 Hz, 1H), 7.38-7.31 (m, 1H), 7.30-7.23 (m, 2H), 5.33-5.21 (m, 1H), 4.90 (d, J = 9.2 Hz, 1H), 3.96- 3.86 (m, 1H), 3.77 (td, J = 19.6, 12.0 Hz, 1H), 2.38-2012 (m, 3H), 2.00-1.90 (m, 1H), 1.33 (d, J = 6.0 Hz, 6H); MS (ES+) m/z 491.2 (M + 1) |
| 355 | 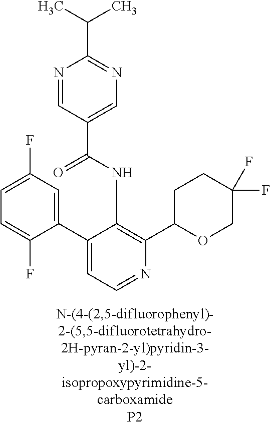 [SMILES: CC(C)c1ncc(C(=O)Nc2c(-c3cc(F)ccc3F)ccnc2C2CCC(F)(F)CO2)cn1] | DIACEL ChiralPak AD Column (250 × 30 mm, 10 μm) eluting with 25% isopropanol containing 0.1% ammonium hydroxide in supercritical carbon dioxide | 1.150 min, 99% ee; (400 MHz, DMSO-d₆) δ 10.39 (s, 1H), 8.98 (s, 2H), 8.68 (d, J = 4.6 Hz, 1H), 7.53 (d, J = 4.8 Hz, 1H), 7.42-7.33 (m, 1H), 7.33-7.24 (m, 2H), 4.93 (d, J = 9.8 Hz, 1H), 4.00-3.72 (m, 2H), 3.20 (td, J = 13.8, 6.8 Hz, 1H), 2.28 (d, J = 10.0 Hz, 3H), 1.96 (d, J = 10.4 Hz, 1H), 1.29 (d, J = 6.8 Hz, 6H); MS (ES+) m/z 475.2 (M + 1) |
| 356 | 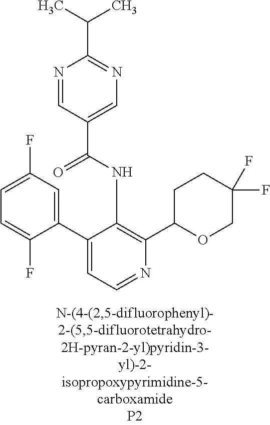 [SMILES: CC(C)c1ncc(C(=O)Nc2c(-c3cc(F)ccc3F)ccnc2C2CCC(F)(F)CO2)cn1] | DIACEL ChiralPak AD Column (250 × 30 mm, 10 μm) eluting with 25% isopropanol containing 0.1% ammonium hydroxide in supercritical carbon dioxide | 1.261 min, 99% ee; (400 MHz, MeOD-d₄) δ 8.99 (s, 2H), 8.67 (d, J = 5.0 Hz, 1H), 7.52 (d, J = 4.8 Hz, 1H), 7.27-7.23 (m, 1H), 7.20 (dd, J = 8.0, 4.8 Hz, 2H), 4.94 (s, 1H), 4.08-3.96 (m, 1H), 3.84-3.70 (m, 1H), 3.30- 3.22 (m, 1H), 2.38-2.25 (m, 2H), 2.21-2.02 (m, 2H), 1.36 (d, J = 6.8 Hz, 6H); MS (ES+) m/z 475.2 (M + 1) |
| 357 | 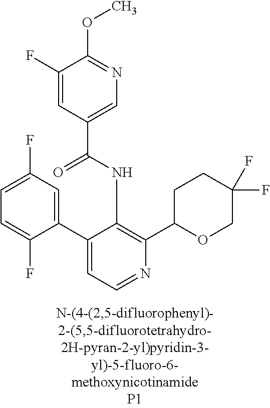 [SMILES: COc1ncc(C(=O)Nc2c(-c3cc(F)ccc3F)ccnc2C2CCC(F)(F)CO2)cc1F] | DIACEL ChiralPak AD Column (250 × 30 mm, 10 μm) eluting with 25% isopropanol containing 0.1% ammonium hydroxide in supercritical carbon dioxide | 0.970 min, 98% ee; (400 MHz, CDCl₃) δ 8.88 (s, 1H), 8.54 (d, J = 5.2 Hz, 1H), 8.34 (d, J = 1.2 Hz, 1H), 7.66 (dd, J = 10.0, 1.6 Hz, 1H), 7.33 (d, J = 4.8 Hz, 1H), 7.15-6.95 (m, 3H), 4.85 (dd, J = 9.6, 4.0 Hz, 1H), 4.18- 4.10 (m, 1H), 4.09 (s, 3H), 3.73 (td, J = 16.8, 12.4 Hz, 1H), 2.41-2.29 (m, 1H), 2.26-2.14 (m, 2H), 2.13-1.98 (m, 1H); MS (ES+) m/z 480.2 (M + 1) |
| 358 | 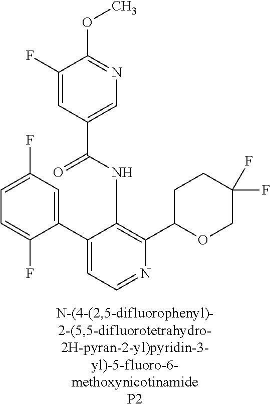 [SMILES: COc1ncc(C(=O)Nc2c(-c3cc(F)ccc3F)ccnc2C2CCC(F)(F)CO2)cc1F] | DIACEL ChiralPak AD Column (250 × 30 mm, 10 μm) eluting with 25% isopropanol containing 0.1% ammonium hydroxide in supercritical carbon dioxide | 1.261 min, 98% ee; (400 MHz, CDCl₃) δ 8.88 (s, 1H), 8.54 (d, J = 5.2 Hz, 1H), 8.34 (d, J = 1.2 Hz, 1H), 7.66 (dd, J = 10.4, 1.6 Hz, 1H), 7.33 (d, J = 4.8 Hz, 1H), 7.14-7.03 (m, 2H), 7.03- 6.96 (m, 1H), 4.85 (dd, J = 4.0, 9.6 Hz, 1H), 4.20-4.11 (m, 1H), 4.09 (s, 3H), 3.82-3.62 (m, 1H), 2.45-2.29 (m, 1H), 2.27- 2.13 (m, 2H), 2.12-1.98 (m, 1H); MS (ES+) m/z 480.2 (M + 1) |
| 359 | 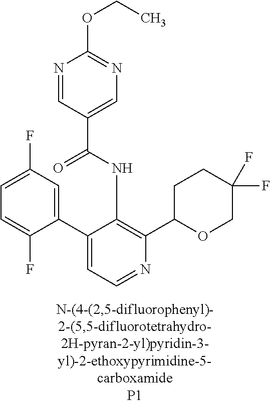 [SMILES: CCOc1ncc(C(=O)Nc2c(-c3cc(F)ccc3F)ccnc2C2CCC(F)(F)CO2)cn1] | DIACEL ChiralPak AD-H Column (250 × 30 mm, 5 μm) eluting with 30% isopropanol containing 0.1% ammonium hydroxide in supercritical carbon dioxide | 1.142 min, 99% ee); (400 MHz, CDCl₃) δ 8.92 (s, 1H), 8.84 (s, 2H), 8.54 (d, J = 5.2 Hz, 1H), 7.33 (d, J = 5.2 Hz, 1H), 7.17- 6.94 (m, 3H), 4.91-4.81 (m, 1H), 4.50 (q, J = 7.2 Hz, 2H), 4.12 (dtd, J = 12.4, 6.4, 2.8 Hz, 1H), 3.82-3.65 (m, 1H), 2.41- 2.27 (m, 1H), 2.16 (dd, J = 7.2, 3.2 Hz, 2H), 2.13-1.93 (m, 1H), 1.46 (t, J = 7.2 Hz, 3H); MS (ES+) m/z 477.3 (M + 1) |
| 360 | 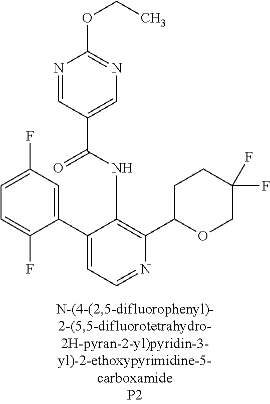 [SMILES: CCOc1ncc(C(=O)Nc2c(-c3cc(F)ccc3F)ccnc2C2CCC(F)(F)CO2)cn1] | DIACEL ChiralPak AD-H Column (250 × 30 mm, 5 μm) eluting with 30% isopropanol containing 0.1% ammonium hydroxide in supercritical carbon dioxide | 1.269 min, 98% ee); (400 MHz, CDCl₃) δ 8.92 (s, 1H), 8.84 (s, 2H), 8.54 (d, J = 5.2 Hz, 1H), 7.33 (d, J = 5.2 Hz, 1H), 7.15- 6.95 (m, 3H), 4.90-4.81 (m, 1H), 4.50 (q, J = 12 Hz, 2H), 4.12 (dtd, J = 12.4, 6.0, 2.4 Hz, 1H), 3.79-3.66 (m, 1H), 2.41- 2.27 (m, 1H), 2.23-2.13 (m, 2H), 2.12-1.95 (m, 1H), 1.46 (t, J = 7.2 Hz, 3H); MS (ES+) m/z 477.3 (M + 1) |
| 361 | 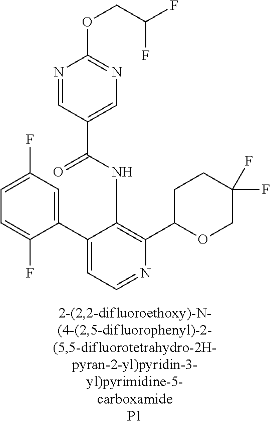 [SMILES: O=C(Nc1c(-c2cc(F)ccc2F)ccnc1C1CCC(F)(F)CO1)c1cnc(OCC(F)F)nc1] | DIACEL ChiralPak IG Column (250 × 30 mm, 10 μm) eluting with 20% ethanol in supercritical carbon dioxide | 1.008 min, 99% ee; (400 MHz, MeOD-d₄) δ 8.90 (s, 2H), 8.64 (d, J = 4.8 Hz, 1H), 7.49 (d, J = 4.8 Hz, 1H), 7.25-7.08 (m, 3H), 6.42-6.06 (m, 1H), 4.91-4.80 (m, 1H), 4.68 (dt, J = 13.6, 3.6 Hz, 2H), 4.06-3.95 (m, 1H), 3.83-3.68 (m, 1H), 2.34-2.02 (m, 4H); MS (ES+) m/z 513.2 (M + 1) |
| 362 | 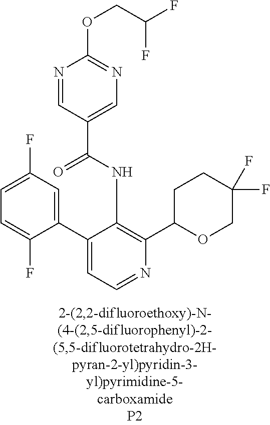 [SMILES: O=C(Nc1c(-c2cc(F)ccc2F)ccnc1C1CCC(F)(F)CO1)c1cnc(OCC(F)F)nc1] | DIACEL ChiralPak IG Column (250 × 30 mm, 10 μm) eluting with 20% ethanol in supercritical carbon dioxide | 1.139 min, 96% ee; (400 MHz, MeOD-d₄) δ 8.91 (s, 2H), 8.65 (d, J = 4.8 Hz, 1H), 7.50 (d, J = 4.8 Hz, 1H), 7.27-7.11 (m, 3H), 6.39-6.09 (m, 1H), 4.92-4.80 (m, 1H), 4.69 (dt, J = 3.6, 13.6 Hz, 2H), 4.59 (s, 1H), 4.08- 3.95 (m, 1H), 3.83-3.68 (m, 1H), 2.35-2.04 (m, 4H); MS (ES+) m/z 513.2 (M + 1) |
| 363 | 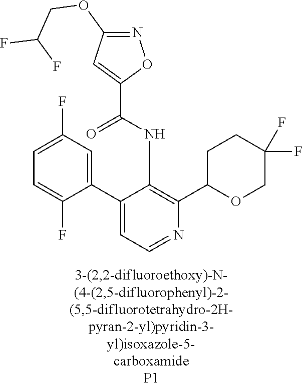 [SMILES: O=C(Nc1c(-c2cc(F)ccc2F)ccnc1C1CCC(F)(F)CO1)c1cc(OCC(F)F)no1] | Chiralcel OD Column (50 × 4.6 mm, 3 μm) eluting with a gradient of 5 to 40% isopropanol with 0.05% diethylamine in supercritical carbon dioxide | 1.070 min, 99% ee; (400 MHz, CDCl₃) δ 9.26 (s, 1H), 8.57 (d, J = 4.8 Hz, 1H), 7.34 (d, J = 4.8 Hz, 1H), 7.15-6.99 (m, 3H), 6.48 (s, 1H), 6.29-5.95 (m, 1H), 4.80 (dd, J = 9.6, 4.0 Hz, 1H), 4.50 (dt, J = 4.0, 13.2 Hz, 2H), 4.27-4.15 (m, 1H), 3.86- 3.70 (m, 1H), 2.47-2.34 (m, 1H), 2.30-2.21 (m, 2H), 2.17- 1.97 (m, 1H); MS (ES+) m/z 502.3 (M + 1) |
| 364 | 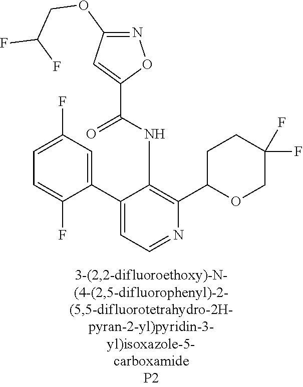 [SMILES: O=C(Nc1c(-c2cc(F)ccc2F)ccnc1C1CCC(F)(F)CO1)c1cc(OCC(F)F)no1] | Chiralcel OD Column (50 × 4.6 mm, 3 μm) eluting with a gradient of 5 to 40% isopropanol with 0.05% diethylamine in supercritical carbon dioxide | Retention time (1.350 min, 99% ee): (400 MHz, CDCl₃) δ 9.26 (s, 1H), 8.57 (d, J = 4.8 Hz, 1H), 7.34 (d, J = 4.8 Hz, 1H), 7.16-6.98 (m, 3H), 6.48 (s, 1H), 6.30-5.94 (m, 1H), 4.80 (dd, J = 4.0, 9.6 Hz, 1H), 4.49 (dt, J = 13.2, 4.0 Hz, 2H), 4.26- 4.14 (m, 1H), 3.85-3.67 (m, 1H). 2.46-2.34 (m, 1H), 2.31- 2.19 (m, 2H), 2.17-1.96 (m, 1H); MS (ES+) m/z 502.3 (M + 1) |
| 365 | 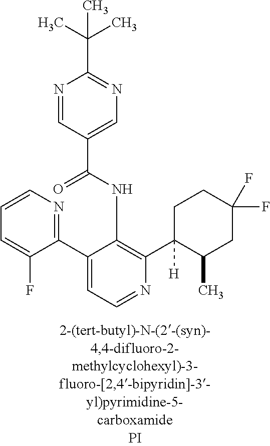 [SMILES: C[C@@H]1CC(F)(F)CC[C@@H]1c1nccc(-c2ncccc2F)c1NC(=O)c1cnc(C(C)(C)C)nc1] | Chiralpak IG Column (250 × 30 mm, 10 μm) eluting with 20% ethanol with 0.1% ammonium hydroxide in supercritical carbon dioxide | 1.052 min, 99% ee; (400 MHz, DMSO-d₆) δ 10.39 (s, 1H), 8.93 (s, 2H), 8.66 (d, J = 4.8 Hz, 1H), 8.47 (d, J = 4.8 Hz, 1H), 7.83 (t, J = 8.8 Hz, 1H), 7.52- 7.46 (m, 2H), 3.49 (s, 1H), 2.46-2.37 (m, 1H), 2.31-2.16 (m, 2H), 2.14-2.06 (m, 1H), 1.99-1.81 (m, 3H), 1.36 (s, 9H), 0.75 (d, J = 7.2 Hz, 3H); MS (ES+) m/z 484.1 (M + 1) |
| 366 | 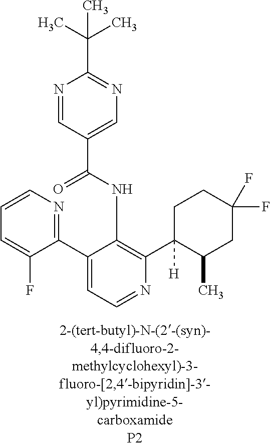 [SMILES: C[C@@H]1CC(F)(F)CC[C@@H]1c1nccc(-c2ncccc2F)c1NC(=O)c1cnc(C(C)(C)C)nc1] | Chiralpak IG Column (250 × 30 mm, 10 μm) eluting with 20% ethanol with 0.1% ammonium hydroxide in supercritical carbon dioxide | 1.285 min, 99% ee; (400 MHz, DMSO-d₆) δ 10.39 (s, 1H), 8.93 (s, 2H), 8.66 (d, J = 4.8 Hz, 1H), 8.47 (d, J = 4.4 Hz, 1H), 7.83 (t, J = 9.6 Hz, 1H), 7.55- 7.44 (m, 2H), 3.49 (d, J = 2.8 Hz, 1H), 2.43 (d, J = 1.2 Hz, 1H), 2.26 (s, 2H), 2.15-2.05 (m, 1H), 2.00-1.80 (m, 3H), 1.36 (s, 9H), 0.75 (d, J = 7.2 Hz, 3H); MS (ES+) m/z 484.1 (M + 1) |
| 367 | 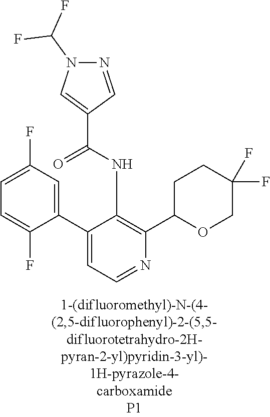 [SMILES: O=C(Nc1c(-c2cc(F)ccc2F)ccnc1C1CCC(F)(F)CO1)c1cnn(C(F)F)c1] | DAICEL Chiralpak AD Column (250 × 30 mm, 5 μm) eluting with 30% isopropanol with 0.1% ammonium hydroxide in supercritical carbon dioxide | 1.169 min, 99% ee; (400 MHz, DMSO-d₆) δ 9.92 (s, 1H), 8.69 (s, 1H), 8.65 (d, J = 4.8 Hz, 1H), 8.15 (s, 1H), 8.03-7.68 (m, 1H), 7.49 (d, J = 4.8 Hz, 1H), 7.37-7.29 (m, 1H), 7.29- 7.20 (m, 2H), 4.92-4.82 (m, 1H), 4.00-3.87 (m, 1H), 3.83- 3.65 (m, 1H), 2.31-2.23 (m, 2H), 2.23-2.08 (m, 1H), 1.94 (d, J = 11.2 Hz, 1H); MS (ES+) m/z 471.0 (M + 1) |
| 368 | 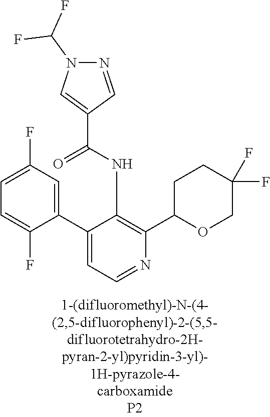 [SMILES: O=C(Nc1c(-c2cc(F)ccc2F)ccnc1C1CCC(F)(F)CO1)c1cnn(C(F)F)c1] | DAICEL Chiralpak AD Column (250 × 30 mm, 5 μm) eluting with 30% isopropanol with 0.1% ammonium hydroxide in supercritical carbon dioxide | 1.332 min, 99% ee; (400 MHz, DMSO-d₆) δ 9.93 (s, 1H), 8.69 (s, 1H), 8.65 (d, J = 4.8 Hz, 1H), 8.15 (s, 1H), 8.04-7.65 (m, 1H), 7.49 (d, J = 4.8 Hz, 1H), 7.36-7.28 (m, 1H), 7.28- 7.20 (m, 2H), 4.87 (d, J = 10.4 Hz, 1H), 3.98-3.86 (m, 1H), 3.81-3.66 (m, 1H), 2.32-2.23 (m, 2H), 2.22-2.10 (m, 1H), 1.99-1.88 (m, 1H); MS (ES+) m/z 471.0 (M + 1) |
| 369 | 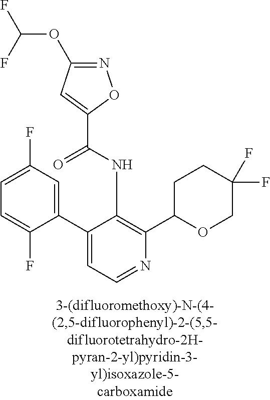 [SMILES: O=C(Nc1c(-c2cc(F)ccc2F)ccnc1C1CCC(F)(F)CO1)c1cc(OC(F)F)no1] | Chiralcel OD Column (50 × 4.6 mm, 3 μm) eluting with a gradient of 5 to 40% ethanol with 0.05% diethylamine in supercritical carbon dioxide | 0.728 min, 99% ee; (400 MHz, CDCl₃) δ 9.33 (s, 1H), 8.58 (d, J = 4.8 Hz, 1H), 7.34 (d, J = 4.2 Hz, 1H), 7.16-6.81 (t, J = 70.8 Hz 1H), 7.14-7.01 (m, 3H), 6.61 (s, 1H), 4.89-4.73 (m, 1H), 4.20 (t, J = 10.4 Hz, 1H), 3.90-3.66 (m, 1H), 2.38 (d, J = 3.2 Hz, 1H), 2.32-2.19 (s, 2H), 2.17-1.97 (m, 1H); MS (ES+) m/z 488.1 (M + 1) |
| 370 | 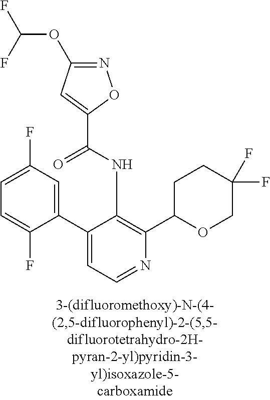 [SMILES: O=C(Nc1c(-c2cc(F)ccc2F)ccnc1C1CCC(F)(F)CO1)c1cc(OC(F)F)no1] | Chiralcel OD Column (50 × 4.6 mm, 3 μm) eluting with a gradient of 5 to 40% ethanol with 0.05% diethylamine in supercritical carbon dioxide | Retention time (0.928 min, 99% ee); (400 MHz, CDCl₃) δ 9.33 (s, 1H), 8.58 (d, J = 4.9 Hz, 1H), 7.34 (d, J = 4.9 Hz, 1H), 7.16-6.81 (t, J = 79.6 Hz 1H), 7.14-7.02 (m, 3H), 6.61 (s, 1H), 4.82 (t, J = 6.8 Hz, 1H), 4.26-4.12 (m, 1H), 3.85-3.69 (m, 1H), 2.38 (dd, J = 6.4, 3.2 Hz, 1H), 2.28-2.17 (m, 2H), 2.17-1.97 (m, 1H); MS (ES+) m/z 488.1 (M + 1) |
| 371 | 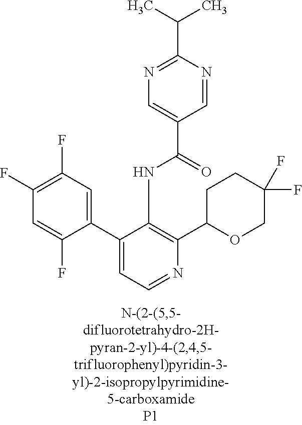 [SMILES: CC(C)c1ncc(C(=O)Nc2c(-c3cc(F)c(F)cc3F)ccnc2C2CCC(F)(F)CO2)cn1] | DAICEL Chiralpak IG Column (250 × 30 mm, 5 μm) eluting with 20% ethanol with 0.1% ammonium hydroxide in supercritical carbon dioxide | 1.062 min, 99% ee; (400 MHz, DMSO-d₆) δ 10.37 (s, 1H), 9.00 (s, 2H), 8.68 (d, J = 4.8 Hz, 1H), 7.69 (dt, J = 10.0, 6.8 Hz, 1H), 7.61-7.54 (m, 1H), 7.52 (d, J = 4.8 Hz, 1H), 4.93 (d, J = 9.2 Hz, 1H), 3.91 (td, J = 11.6, 5.6 Hz, 1H), 3.86-3.73 (m, 1H), 3.20 (td, J = 13.6, 6.8 Hz, 1H), 2.37-2.17 (m, 3H), 2.00-1.91 (m, 1H), 1.29 (d, J = 6.8 Hz, 6H); MS (ES+) m/z 493.2 (M + 1) |
| 372 | 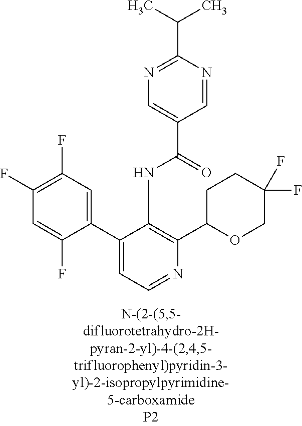 [SMILES: CC(C)c1ncc(C(=O)Nc2c(-c3cc(F)c(F)cc3F)ccnc2C2CCC(F)(F)CO2)cn1] | DAICEL Chiralpak IG Column (250 × 30 mm, 5 μm) eluting with 20% ethanol with 0.1% ammonium hydroxide in supercritical carbon dioxide | 1.183 min, 99% ee; (400 MHz, DMSO-d₆) δ 10.37 (s, 1H), 9.00 (s, 2H), 8.68 (d, J = 4.8 Hz, 1H), 7.69 (dt, J = 10.0, 6.8 Hz, 1H), 7.60-7.54 (m, 1H), 7.52 (d, J = 4.8 Hz, 1H), 4.93 (d, J = 8.8 Hz, 1H), 3.95-3.87 (m, 1H), 3.86-3.74 (m, 1H), 3.20 (td, J = 13.8, 7.0 Hz, 1H), 2.36-2.18 (m, 3H), 1.95 (dd, J = 10.0, 1.6 Hz, 1H), 1.29 (d, J = 7.2 Hz, 6H); MS (ES+) m/z 493.2 (M + 1) |
| 373 | 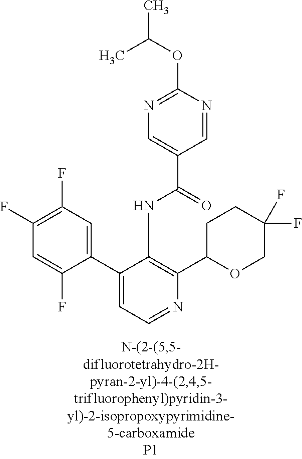 [SMILES: CC(C)Oc1ncc(C(=O)Nc2c(-c3cc(F)c(F)cc3F)ccnc2C2CCC(F)(F)CO2)cn1] | DAICEL Chiralpak OJ Column (250 × 30 mm, 10 μm) eluting with 20% methanol with 0.1% ammonium hydroxide in supercritical carbon dioxide | 0.869 min, 99% ee; (400 MHz, DMSO-d₆) δ 10.20 (s, 1H), 8.89 (s, 2H), 8.67 (d, J = 4.8 Hz, 1H), 7.67 (dt, J = 10.4, 6.8 Hz, 1H), 7.59-7.53 (m, 1H), 7.51 (d, J = 4.4 Hz, 1H), 5.27 (td, J = 12.4, 6.0 Hz, 1H), 4.91 (d, J = 9.6 Hz, 1H), 3.96-3.86 (m, 1H), 3.78 (td, J = 19.2, 12.4 Hz, 1H), 3.32 (s, 4H), 2.36-2.15 (m, 3H), 1.95 (d, J = 11.2 Hz, 1H), 1.34 (d, J = 6.4 Hz, 6H); MS (ES+) m/z 509.2 (M + 1). |
| 374 | 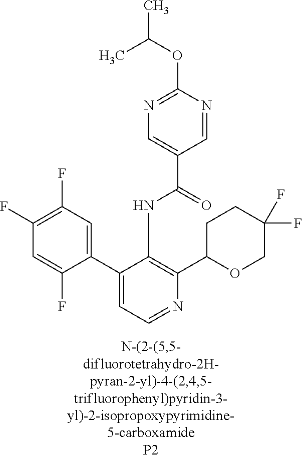 [SMILES: CC(C)Oc1ncc(C(=O)Nc2c(-c3cc(F)c(F)cc3F)ccnc2C2CCC(F)(F)CO2)cn1] | DAICEL Chiralpak OJ Column (250 × 30 mm, 10 μm) eluting with 20% methanol with 0.1% ammonium hydroxide in supercritical carbon dioxide | 1.129 min, 99% ee; (400 MHz, DMSO-d₆) δ 10.20 (s, 1H), 8.89 (s, 2H), 8.66 (d, J = 4.8 Hz, 1H), 7.67 (dt, J = 10.4, 6.8 Hz, 1H), 7.60-7.53 (m, 1H), 7.51 (d, J = 4.8 Hz, 1H), 5.33-5.23 (m, 1H), 4.91 (d, J = 10.4 Hz, 1H), 3.93-3.86 (m, 1H),3.85- 3.71 (m, 1H), 2.36-2.17 (m, 3H), 1.95 (d, J = 10.4 Hz, 1H), 1.34 (d, J = 6.0 Hz, 6H); MS (ES+) m/z 509.2 (M + 1) |
| 375 | 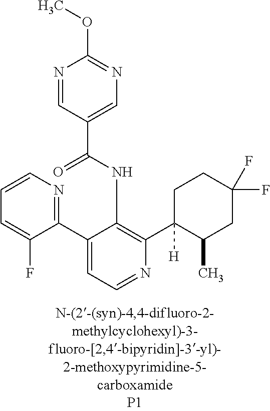 [SMILES: COc1ncc(C(=O)Nc2c(-c3ncccc3F)ccnc2[C@H]2CCC(F)(F)C[C@H]2C)cn1] | DAICEL Chiralpak OJ Column (250 × 30 mm, 10 μm) eluting with 25% methanol with 0.1% ammonium hydroxide in supercritical carbon dioxide | 1.080 min, 99% ee; (400 MHz, CDCl₃) δ 9.89 (s, 1H), 9.00- 8.99 (m, 2H), 8.68 (d, J = 5.0 Hz, 1H), 8.54-8.52 (m, 1H), 7.64 (ddd, J = 9.9, 8.7, 1.2 Hz, 1H), 7.51 (dd, J = 4.9, 3.6 Hz, 1H), 7.42 (dt, J = 8.5, 4.3 Hz, 1H), 4.11 (d, J = 4.9 Hz, 3H), 3.39 (t, J = 4.0 Hz, 1H), 2.64- 2.51 (m, 2H), 2.38-2.30 (m, 2H), 2.09-2.02 (m, 1H), 1.97- 1.86 (m, 2H), 0.86-0.83 (m, 3H); MS (ES+) m/z 458.2 (M + 1) |
| 376 | 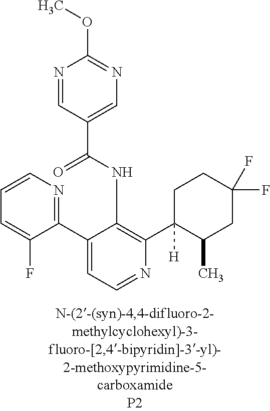 [SMILES: COc1ncc(C(=O)Nc2c(-c3ncccc3F)ccnc2[C@H]2CCC(F)(F)C[C@H]2C)cn1] | DAICEL Chiralpak OJ Column (250 × 30 mm, 10 μm) eluting with 25% methanol with 0.1% ammonium hydroxide in supercritical carbon dioxide | 1.315 min, 99% ee; (400 MHz, CDCl₃) δ 9.91-9.88 (m, 1H), 9.01-8.98 (m, 2H), 8.68 (d, J = 5.0 Hz, 1H), 8.53-8.52 (m, 1H), 7.63 (td, J = 9.3, 1.2 Hz, 1H), 7.51 (dd, J = 4.9, 3.6 Hz, 1H), 7.43-7.39 (m, 1H), 4.11-4.09 (m, 3H), 3.41-3.36 (m, 1H), 2.65-2.50 (m, 2H), 2.39-2.29 (m, 2H), 2.10-2.01 (m, 1H), 1.98-1.85 (m, 2H), 0.86-0.78 (m, 3H); MS (ES+) m/z 458.2 (M + 1) |

### Example 377

### Synthesis of N-(2-(4,4-difluorocyclohexyl)-4-(2,4-difluorophenyl)pyridin-3-yl)-2-isopropylpyrimidine-5-carboxamide

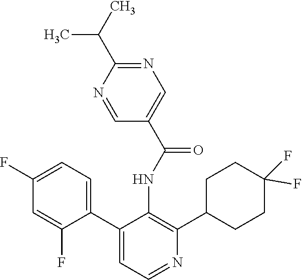 [SMILES: CC(C)c1ncc(C(=O)Nc2c(-c3ccc(F)cc3F)ccnc2C2CCC(F)(F)CC2)cn1]

### Step 1. Preparation of N-[2-chloro-4-(2,4-difluorophenyl)-3-pyridyl]-2-isopropyl-pyrimidine-5-carboxamide

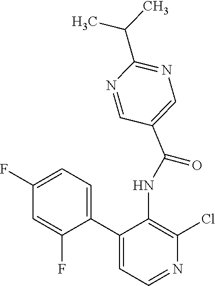 [SMILES: CC(C)c1ncc(C(=O)Nc2c(-c3ccc(F)cc3F)ccnc2Cl)cn1]

A mixture of N-(2-chloro-4-iodopyridin-3-yl)-2-isopropylpyrimidine-5-carboxamide (0.090 g, 0.22 mmol) in dioxane (1.2 mL) and water (0.11 mL) was degassed with nitrogen for 10 minutes. To the reaction mixture was added potassium carbonate (0.092 g, 0.67 mmol), 2-(3,4-dihydro-2H-pyran-6-yl)-4,4,5,5-tetramethyl-1,3,2-dioxaborolane (0.070 g, 0.34 mmol), [1,1′-Bis(diphenylphosphino)ferrocene]dichloropalladium(II), complex with CH₂Cl₂ (1:1) (0.019 g, 0.022 mmol), and the reaction mixture was stirred at 90° C. for 3 h. After cooling to ambient temperature, the mixture was filtered through a bed of Celite, and the filter pad was washed with ethyl acetate (2×20 mL). The filtrate was washed with saturated ammonium chloride (2×25 mL) and concentrated in vacuo. The residue was purified by column chromatography, eluting with a gradient of 10 to 100% of ethyl acetate in heptane, to afford the title compound as a yellow solid (0.070 g, 79% yield): MS (ES+) m/z 389.2 (M+1), 391.2 (M+1).

### Step 2. Preparation of N-(2-(4,4-difluorocyclohexyl)-4-(2,4-difluorophenyl)pyridin-3-yl)-2-isopropylpyrimidine-5-carboxamide

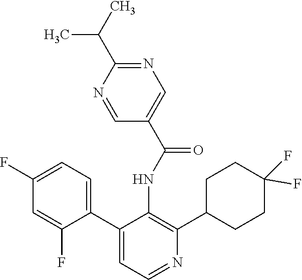 [SMILES: CC(C)c1ncc(C(=O)Nc2c(-c3ccc(F)cc3F)ccnc2C2CCC(F)(F)CC2)cn1]

To a mixture of (4,4″-di-t-butyl-2,2″-bipyridine)bis[3,5-difluoro-2-[5-trifluoromethyl-2-pyridinyl-kN)phenyl-kC]iridium(II) hexafluorophosphate (0.0021 g, 0.0019 mmol), dichloro(dimethoxyethane)nickel (0.0042 g, 0.019 mmol), 4,4′-di-tert-butyl-2,2′-bipyridine (0.0076 g, 0.028 mmol), N-[2-chloro-4-(2,4-difluorophenyl)-3-pyridyl]-2-isopropyl-pyrimidine-5-carboxamide (0.074 g, 0.19 mmol), cesium carbonate (0.12 g, 0.38 mmol) and 4,4-difluorocyclohexanecarboxylic acid (0.0562 g, 0.38 mmol) was added N,N-dimethylformamide (4.7 mL). The mixture was purged with nitrogen for 10 s and sealed. The reaction mixture was then stirred in front of Kessil PR160L lights (440 nm) for 20 h. The reaction mixture was quenched with saturated aqueous sodium bicarbonate solution (10 mL) and extracted with ethyl acetate (3×10 mL). The combined organic extracts were washed with water (3×25 mL) and brine (50 mL), dried over anhydrous sodium sulfate, and concentrated in vacuo. Purification of the residue by preparative reverse phase HPLC, using 5 to 85% of acetonitrile in water containing 0.5% of formic acid as eluent, afforded the title compound as a colorless solid (0.040 g, 45% yield): ¹H-NMR (400 MHz; DMSO-d₆) δ 10.34 (s, 1H), 8.97 (s, 2H), 8.61 (d, J=4.9 Hz, 1H), 7.45-7.34 (m, 3H), 7.18-7.14 (m, 1H), 3.20 (7, J=6.9 Hz, 2H), 2.09-1.85 (m, 8H), 1.29 (d, J=6.9 Hz, 6H); MS (ES+) m/z 473.2 (M+1) In a similar manner as described in examples disclosed herein, utilizing the appropriately substituted starting materials and intermediates, the following compounds were prepared:

| Example | Structure | Amount | | |
|---|---|---|---|---|
| No. | Name | Yield | MS (ES+) m/z | ¹H NMR |
| 378 | 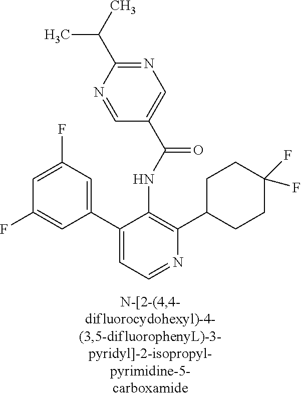 [SMILES: CC(C)c1ncc(C(=O)Nc2c(-c3cc(F)cc(F)c3)ccnc2C2CCC(F)(F)CC2)cn1] | 0.0072 g 18% | 473.2 (M + 1) | (400 MHz, DMSO-d₆) δ 10.40 (s, 1H), 9.04 (s, 2H), 8.63 (d, J = 4.9 Hz, 1H), 7.39 (d, J = 4.9 Hz, 1H), 7.29 (tt, J = 9.4, 2.3 Hz, 1H), 7.24-7.17 (m, 2H), 3.22 (tt, J = 13.8, 6.9 Hz, 2H), 2.09- 2.02 (m, 3H), 1.93-1.79 (m, 5H), 1.30 (d, J = 6.9 Hz, 6H) |
| 379 | 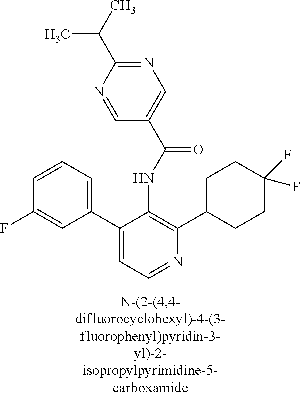 [SMILES: CC(C)c1ncc(C(=O)Nc2c(-c3cccc(F)c3)ccnc2C2CCC(F)(F)CC2)cn1] | 0.071 g 50% | 455.2 (M + 1) | ¹H-NMR (400 MHz; DMSO-d₆) δ 10.36 (s, 1H), 9.02 (s, 2H), 8.62 (d, J = 4.9 Hz, 1H), 7.47 (td, J = 8.1, 6.2 Hz, 1H), 7.37 (d, J = 4.9 Hz, 1H), 7.32-7.28 (m, 2H), 7.25- 7.19 (m, 1H), 3.21 (7, J = 6.9 Hz, 2H), 2.08-1.82 (m, 8H), 1.30 (d, J = 6.9 Hz, 6H) |
| 380 | 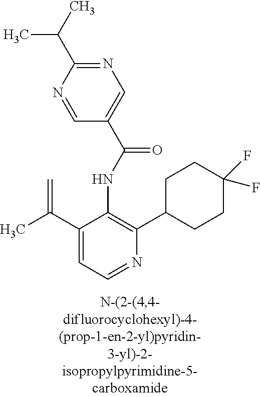 [SMILES: C=C(C)c1ccnc(C2CCC(F)(F)CC2)c1NC(=O)c1cnc(C(C)C)nc1] | 0.033 g 46% | 401.2 (M + 1) | ¹H-NMR (400 MHz; DMSO-d₆) δ 10.24 (s, 1H), 9.19 (s, 2H), 8.48 (d, J = 4.9 Hz, 1H), 7.22 (d, J = 4.9 Hz, 1H), 5.19 (s, 1H), 5.00 (s, 1H), 3.29-3.21 (m, 1H), 3.19- 3.10 (m, 1H), 2.05-2.02 (m, 2H), 1.99 (s, 3H), 1.88-1.80 (m, 5H), 1.33 (d, J = 6.9 Hz, 6H) |

### Example 381

### Synthesis of N-(2-(4,4-difluorocyclohexyl)-4-(2-(4,4-difluorocyclohexyl)phenyl)pyridin-3-yl)-2-isopropylpyrimidine-5-carboxamide

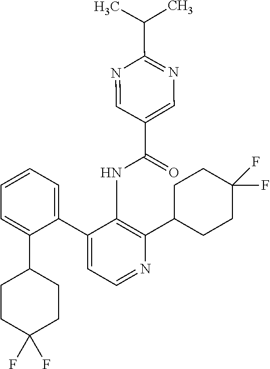 [SMILES: CC(C)c1ncc(C(=O)Nc2c(-c3ccccc3C3CCC(F)(F)CC3)ccnc2C2CCC(F)(F)CC2)cn1]

### Step 1. Preparation of N-[2-chloro-4-(2-chlorophenyl)-3-pyridyl]-2-isopropyl-pyrimidine-5-carboxamide

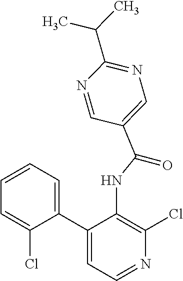 [SMILES: CC(C)c1ncc(C(=O)Nc2c(-c3ccccc3Cl)ccnc2Cl)cn1]

A mixture of N-(2-chloro-4-iodopyridin-3-yl)-2-isopropylpyrimidine-5-carboxamide (0.10 g, 0.25 mmol) in dioxane (1.8 mL) and water (0.20 mL) was degassed with nitrogen for 10 minutes. To the reaction mixture was added potassium carbonate (0.10 g, 0.75 mmol), 2-chlorophenylboronic acid (0.058 g, 0.37 mmol), [1,1′-bis(diphenylphosphino)ferrocene]dichloropalladium(II), complex with CH₂Cl₂ (1:1) (0.020 g, 0.025 mmol), and the reaction mixture was stirred at 90° C. for 3 h. After cooling to ambient temperature, the mixture was filtered through a bed of Celite and the filter pad was washed with ethyl acetate (2×20 mL). The combined filtrate was washed with saturated ammonium chloride (2×25 mL) and concentrated in vacuo. The residue was purified by column chromatography, eluting with a gradient of 5 to 100% ethyl acetate in heptane, to afford the title compound as a yellow solid (0.065 g, 68% yield): MS (ES+) m/z 387.0 (M+1), 389.0 (M+1).

### Step 2. Preparation of N-(2-(4,4-difluorocyclohexyl)-4-(2-(4,4-difluorocyclohexyl)phenyl)pyridin-3-yl)-2-isopropylpyrimidine-5-carboxamide

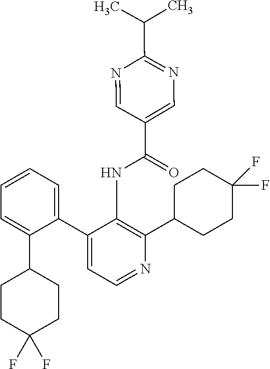 [SMILES: CC(C)c1ncc(C(=O)Nc2c(-c3ccccc3C3CCC(F)(F)CC3)ccnc2C2CCC(F)(F)CC2)cn1]

To a mixture of (4,4″-di-t-butyl-2,2″-bipyridine)bis[3,5-difluoro-2-[5-trifluoromethyl-2-pyridinyl kN)phenyl-kC]iridium(III) hexafluorophosphate (0.0017 g, 0.0015 mmol), dichloro(dimethoxyethane)nickel (0.0034 g, 0.015 mmol), 4,4′-di-tert-butyl-2,2′-bipyridine (0.0062 g, 0.023 mmol), N-[2-chloro-4-(2-chlorophenyl)-3-pyridyl]-2-isopropyl-pyrimidine-5-carboxamide (0.060 g, 0.15 mmol), cesium carbonate (0.10 g, 0.31 mmol) and 4,4-difluorocyclohexanecarboxylic acid (0.051 g, 0.31 mmol) was added N,N-dimethylformamide (3.9 mL). The mixture was purged with nitrogen for 10 s and sealed. The reaction mixture was then stirred in front of Kessil PR160L lights (440 nm) for 20 h. The reaction mixture was quenched with saturated aqueous sodium bicarbonate solution (10 mL) and extracted with ethyl acetate (3×10 mL). The combined organic extracts were washed with water (3×25 mL) and brine (50 mL), dried over anhydrous sodium sulfate, and concentrated in vacuo. Purification of the residue by column chromatography, eluting with a gradient of 5 to 50% ethyl acetate afforded the title compound as a colorless solid (0.030 g, 32% yield): ¹H-NMR (400 MHz; DMSO-d₆) δ 10.12 (s, 1H), 8.80 (s, 2H), 8.57 (d, J=4.9 Hz, 1H), 7.35-7.29 (m, 2H), 7.23-7.20 (m, 2H), 7.13-7.11 (m, 1H), 3.16 (dquintet, J=13.8, 6.9 Hz, 2H), 2.09-1.62 (m, 17H), 1.26 (d, J=6.9 Hz, 6H); MS (ES+) m/z 555.2 (M+1) δ 57.2 (M+1).

### Example 382

### Synthesis of N-(4-cyclopropyl-2-(4,4-difluorocyclohexyl)pyridin-3-yl)-2-isopropylpyrimidine-5-carboxamide

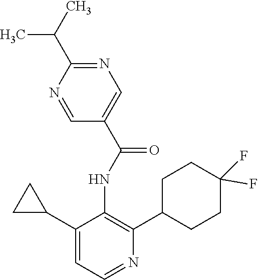 [SMILES: CC(C)c1ncc(C(=O)Nc2c(C3CC3)ccnc2C2CCC(F)(F)CC2)cn1]

### Step 1. Preparation of N-(2-chloro-4-cyclopropylpyridin-3-yl)-2-isopropylpyrimidine-5-carboxamide

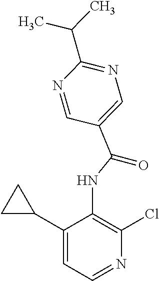 [SMILES: CC(C)c1ncc(C(=O)Nc2c(C3CC3)ccnc2Cl)cn1]

A mixture of N-(2-chloro-4-iodopyridin-3-yl)-2-isopropylpyrimidine-5-carboxamide (0.10 g, 0.25 mmol) in anhydrous (2.0 mL) was degassed with nitrogen for 10 minutes. To the reaction mixture was added tetrakis(triphenylphosphine)palladium(0) (0.029 g, 0.025 mmol), cyclopropylzinc bromide (0.99 mL, 0.50 mmol), and the reaction mixture was stirred at 70° C. for 3 h. After cooling to ambient temperature, the mixture was partitioned between ethyl acetate (2×20 mL) and water (20 mL). The organic layer was washed with saturated ammonium chloride (2×25 mL), water (25 mL), and brine (25 mL), and then concentrated in vacuo. Purification of the residue by column chromatography, eluting with a gradient of 5 to 95% ethyl acetate in heptane afforded the title compound as a yellow solid. The residue was used in the next step without further purification (0.043 g, 55% yield): MS (ES+) m/z 317.0 (M+1) 319.0 (M+1).

### Step 2. Preparation of N-(4-cyclopropyl-2-(4,4-difluorocyclohexyl)pyridin-3-yl)-2-isopropylpyrimidine-5-carboxamide

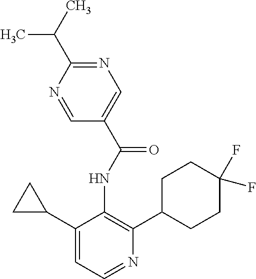 [SMILES: CC(C)c1ncc(C(=O)Nc2c(C3CC3)ccnc2C2CCC(F)(F)CC2)cn1]

To a mixture of (4,4″-di-t-butyl-2,2″-bipyridine)bis[3,5-difluoro-2-[5-trifluoromethyl-2-pyridinyl kN)phenyl-kC]iridium(III) hexafluorophosphate (0.0010 g, 0.90 mmol), dichloro(dimethoxyethane)nickel (0.0019 g, 0.0088 mmol), 4,4′-di-tert-butyl-2,2′-bipyridine (0.0036 g, 0.013 mmol), N-(2-chloro-4-cyclopropylpyridin-3-yl)-2-isopropylpyrimidine-5-carboxamide (0.028 g, 0.088 mmol), cesium carbonate (0.58 g, 0.18 mmol) and 4,4-difluorocyclohexanecarboxylic acid (0.029 g, 0.18 mmol) was added N,N-dimethylformamide (2.2 mL). The mixture was purged with nitrogen for 10 s and sealed. The reaction mixture was then stirred in front of Kessil PR160L lights (440 nm) for 20 h. The reaction mixture was quenched with saturated aqueous sodium bicarbonate solution (10 mL) and extracted with ethyl acetate (3×10 mL). The combined organic extracts were washed with water (3×25 mL) and brine (50 mL), dried over anhydrous sodium sulfate, and concentrated in vacuo. Purification of the residue by preparative reverse-phase HPLC, eluting with 20 to 65% acetonitrile in water containing 0.5% formic acid afforded the title compound as a colorless solid (0.0060 g, 16% yield): ¹H-NMR (400 MHz; DMSO-d₆) δ 10.37 (s, 1H), 9.28 (s, 2H), 8.36 (d, J=5.1 Hz, 1H), 6.81 (d, J=5.2 Hz, 1H), 3.24 (td, J=13.7, 6.8 Hz, 1H), 3.13-3.05 (m, 1H), 2.04-1.94 (m, 4H), 1.86-1.75 (m, 5H), 1.33 (d, J=6.9 Hz, 6H), 0.99 (dq, J=7.8, 3.7 Hz, 2H), 0.76-0.72 (m, 2H); MS (ES+) m/z 401.2 (M+1).

### Example 383

### Synthesis of N-(4-(cyclohex-1-en-1-yl)-2-(4,4-difluorocyclohexyl)pyridin-3-yl)-2-isopropylpyrimidine-5-carboxamide

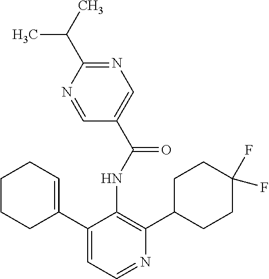 [SMILES: CC(C)c1ncc(C(=O)Nc2c(C3=CCCCC3)ccnc2C2CCC(F)(F)CC2)cn1]

### Step 1. Preparation of N-(2-chloro-4-(cyclohex-1-en-1-yl)pyridin-3-yl)-2-isopropylpyrimidine-5-carboxamide

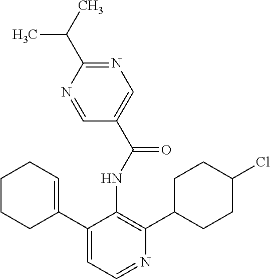 [SMILES: CC(C)c1ncc(C(=O)Nc2c(C3=CCCCC3)ccnc2C2CCC(Cl)CC2)cn1]

A mixture of N-(2-chloro-4-iodopyridin-3-yl)-2-isopropylpyrimidine-5-carboxamide (0.20 g, 0.50 mmol) in dioxane (2.2 mL) and water (0.25 mL) was degassed with nitrogen for 10 minutes. To the reaction mixture was added potassium carbonate (0.20 g, 1.5 mmol), cyclohexen-1-ylboronic acid (0.094 g, 0.74 mmol), [1,1′-bis(diphenylphosphino)ferrocene]dichloropalladium(II), complex with CH₂Cl₂ (1:1) (0.040 g, 0.050 mmol), and the reaction mixture was stirred at 90° C. for 3 h. After cooling to ambient temperature, the mixture was filtered through a bed of Celite, and the filter pad was washed with ethyl acetate (2×20 mL). The filtrate was washed with saturated ammonium chloride (2×25 mL) and concentrated in vacuo. The residue was purified by column chromatography, eluting with a gradient of 5 to 100% ethyl acetate in heptane, to afford the title compound as a yellow solid (0.17 g, 93% yield): MS (ES+) m/z 357.0 (M+1), 359.0 (M+1).

### Step 2. Preparation of N-(4-(cyclohex-1-en-1-yl)-2-(4,4-difluorocyclohexyl)pyridin-3-yl)-2-isopropylpyrimidine-5-carboxamide

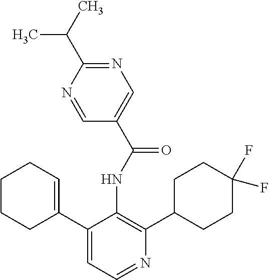 [SMILES: CC(C)c1ncc(C(=O)Nc2c(C3=CCCCC3)ccnc2C2CCC(F)(F)CC2)cn1]

To a mixture of (4,4″-di-t-butyl-2,2″-bipyridine)bis[3,5-difluoro-2-[5-trifluoromethyl-2-pyridinyl kN)phenyl-kC]iridium(III) hexafluorophosphate (0.0050 g, 0.0046 mmol), dichloro(dimethoxyethane)nickel (0.010 g, 0.046 mmol), 4,4′-di-tert-butyl-2,2′-bipyridine (0.019 g, 0.069 mmol), N-(2-chloro-4-(cyclohex-1-en-1-yl)pyridin-3-yl)-2-isopropylpyrimidine-5-carboxamide (0.17 g, 0.46 mmol), cesium carbonate (0.30 g, 0.92 mmol) and 4,4-difluorocyclohexanecarboxylic acid (0.15 g, 0.92 mmol) was added N,N-dimethylformamide (12 mL). The mixture was purged with nitrogen for 10 s and sealed. The reaction mixture was then stirred in front of Kessil PR160L lights (440 nm) for 20 h. The reaction mixture was quenched with saturated aqueous sodium bicarbonate solution (10 mL) and extracted with ethyl acetate (3×20 mL). The combined organic extracts were washed with water (3×50 mL) and brine (50 mL), dried over anhydrous sodium sulfate, and concentrated in vacuo. Purification of the residue by preparative reverse-phase HPLC, eluting with a gradient of 10 to 90% acetonitrile in water containing 0.5% formic acid afforded the title compound as a colorless solid (0.11 g, 53% yield): ¹H-NMR (400 MHz; DMSO-d₆) δ 10.18 (s, 1H), 9.17 (s, 2H), 8.44 (d, J=4.9 Hz, 1H), 7.14 (d, J=4.9 Hz, 1H), 5.71 (dt, J=3.5, 1.9 Hz, 1H), 3.25 (7, J=6.9 Hz, 1H), 3.15-3.09 (m, 1H), 2.19 (s, 2H), 2.06-1.94 (m, 5H), 1.89-1.77 (m, 5H), 1.63-1.48 (m, 4H), 1.33 (d, J=6.9 Hz, 6H); MS (ES+) m/z 441.3 (M+1).

### Example 384

### Synthesis of N-(4-cyclohexyl-2-(4,4-difluorocyclohexyl)pyridin-3-yl)-2-isopropylpyrimidine-5-carboxamide

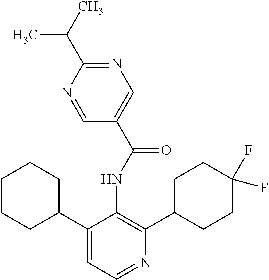 [SMILES: CC(C)c1ncc(C(=O)Nc2c(C3CCCCC3)ccnc2C2CCC(F)(F)CC2)cn1]

To a mixture of N-(4-(cyclohex-1-en-1-yl)-2-(4,4-difluorocyclohexyl)pyridin-3-yl)-2-isopropylpyrimidine-5-carboxamide (0.078 g, 0.18 mmol) in methanol (1.0 mL) and ethyl acetate (1.0 mL) was added ammonium formate (0.22 g, 3.6 mmol) and 10% palladium on carbon (0.028 g). The reaction mixture was stirred at 65° C. for 3 h. After cooling to ambient temperature, the mixture was diluted with ethyl acetate (10 mL), filtered through a bed of Celite and the filtrate was concentrated in vacuo. The residue was purified by preparative reverse-phase HPLC, using a gradient of 10 to 90% acetonitrile in water containing 0.5% formic acid, to afford the title compound as a colorless solid (0.037 g, 47% yield): ¹H NMR (400 MHz, DMSO-d₆) δ 10.27 (s, 1H), 9.25 (s, 2H), 8.45 (d, J=5.1 Hz, 1H), 7.26 (d, J=5.1 Hz, 1H), 3.26 (tt, J=13.8, 6.9 Hz, 1H), 3.11-3.04 (m, 1H), 2.72-2.67 (m, 1H), 2.04-1.66 (m, 13H), 1.44-1.19 (m, 11H); MS (ES+) m/z 443.3 (M+1).

### Example 385

### Synthesis of N-(2-(4,4-difluorocyclohexyl)-4-(pyrimidin-4-yl)pyridin-3-yl)-2-isopropylpyrimidine-5-carboxamide

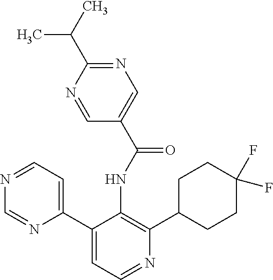 [SMILES: CC(C)c1ncc(C(=O)Nc2c(-c3ccncn3)ccnc2C2CCC(F)(F)CC2)cn1]

### Step 1. Preparation of N-(2-chloro-4-(pyrimidin-4-yl)pyridin-3-yl)-2-isopropylpyrimidine-5-carboxamide

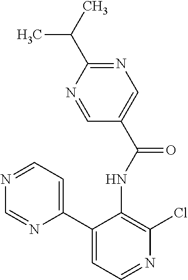 [SMILES: CC(C)c1ncc(C(=O)Nc2c(-c3ccncn3)ccnc2Cl)cn1]

To a solution of N-(2-chloro-4-iodopyridin-3-yl)-2-isopropylpyrimidine-5-carboxamide (0.20 g, 0.50 mmol) in anhydrous dimethylformamide (1.2 mL) was added 4-(tributylstannyl)pyrimidine (0.20 g. 0.55 mmol), copper(I) iodide (0.0095 g, 0.050 mmol) and tetrakis(triphenylphosphine)palladium (0) (0.034 g. 0.030 mmol). The reaction mixture was degassed with nitrogen for 10 minutes and stirred at 115° C. for 3 h. After cooling to ambient temperature, the mixture was filtered through a bed of Celite and the filter pad was washed with ethyl acetate (2×20 mL). The combined filtrates were washed with saturated ammonium chloride (2×25 mL), water (25 mL) and concentrated in vacuo. The residue was purified by column chromatography, eluting with a gradient of 0 to 60% ethyl acetate in heptane, to afford the title compound as a yellow solid (0.086 g, 49% yield): MS (ES+) m/z 355.3 (M+1), 357.2 (M+1).

### Step 2. Preparation of N-(2-(4,4-difluorocyclohexyl)-4-(pyrimidin-4-yl)pyridin-3-yl)-2-isopropylpyrimidine-5-carboxamide

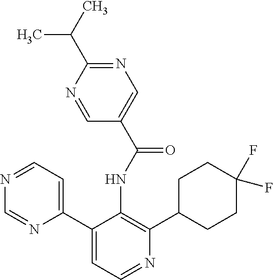 [SMILES: CC(C)c1ncc(C(=O)Nc2c(-c3ccncn3)ccnc2C2CCC(F)(F)CC2)cn1]

To a mixture of (4,4″-di-t-butyl-2,2″-bipyridine)bis[3,5-difluoro-2-[5-trifluoromethyl-2-pyridinyl kN)phenyl-kC]iridium(III) hexafluorophosphate (0.0027 g, 0.0024 mmol), dichloro(dimethoxyethane)nickel (0.0053 g, 0.024 mmol), 4,4′-di-tert-butyl-2,2′-bipyridine (0.0096 g, 0.036 mmol), N-(2-chloro-4-(pyrimidin-4-yl)pyridin-3-yl)-2-isopropylpyrimidine-5-carboxamide (0.085 g, 0.24 mmol), cesium carbonate (0.16 g, 0.48 mmol) and 4,4-difluorocyclohexanecarboxylic acid (0.079 g, 0.48 mmol) was added N,N-dimethylformamide (6.0 mL). The mixture was purged with nitrogen for 10 s and sealed. The reaction mixture was then stirred in front of Kessil PR160L lights (440 nm) for 20 h. The reaction mixture was quenched with saturated aqueous sodium bicarbonate solution (10 mL) and extracted with ethyl acetate (3×20 mL). The combined organic extracts were washed with water (3×50 mL) and brine (50 mL), dried over anhydrous sodium sulfate, and concentrated in vacuo. Purification of the residue by column chromatography, eluting with 5 to 70% ethyl acetate in heptane afforded the title compound as a colorless solid (0.021 g, 20% yield): ¹H-NMR (400 MHz; DMSO-d₆) δ 10.52 (s, 1H), 9.26 (d, J=1.3 Hz, 1H), 9.08 (s, 2H), 8.89 (d, J=5.2 Hz, 1H), 8.72 (d, J=4.9 Hz, 1H), 7.76 (dd, J=5.2, 1.3 Hz, 1H), 7.58 (d, J=4.9 Hz, 1H), 3.29-3.17 (m, 2H), 2.12-1.81 (m, 8H), 1.31 (d, J=6.9 Hz, 6H); MS (ES+) m/z 439.2 (M+1).

In a similar manner as described in examples disclosed herein, utilizing the appropriately substituted starting materials and intermediates, the following compounds were prepared:

| Example | Structure | Amount | | |
|---|---|---|---|---|
| No. | Name | Yield | MS (ES+) m/z | ¹H NMR |
| 386 | 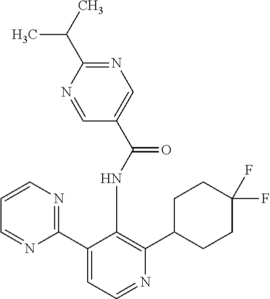 [SMILES: CC(C)c1ncc(C(=O)Nc2c(-c3ncccn3)ccnc2C2CCC(F)(F)CC2)cn1] N-(2-(4,4- difluorocyclohexyl)-4- (pyrimidin-2-yl)pyridin-3- yl)-2-isopropylpyrimidine-5- carboxamide | 0.10 g 57% | 439.2 (M + 1) | (400 MHz, DMSO-d₆) δ 10.47 (s, 1H), 9.10 (s, 2H), 8.89 (d, J = 4.9 Hz, 2H), 8.69 (d, J = 4.9 Hz, 1H), 7.79 (d, J = 4.9 Hz, 1H), 7.49 (t, J = 4.9 Hz, 1H), 3.30-3.18 (m, 2H), 2.10-1.90 (m, 8H), 1.32 (d, J = 6.9 Hz, 6H) |

### Example 387

### Synthesis of N-(2′-(4,4-difluorocyclohex-1-en-1-yl)-[2,4′-bipyridin]-3′-yl)-2-isopropylpyrimidine-5-carboxamide

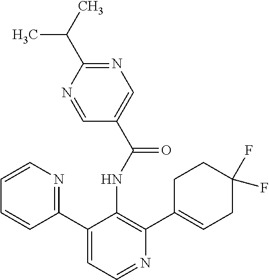 [SMILES: CC(C)c1ncc(C(=O)Nc2c(-c3ccccn3)ccnc2C2=CCC(F)(F)CC2)cn1]

### Step 1. Preparation of N-(2′-chloro-[2,4′-bipyridin]-3′-yl)-2-isopropylpyrimidine-5-carboxamide

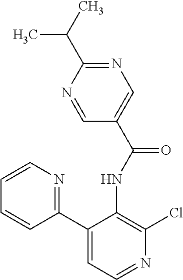 [SMILES: CC(C)c1ncc(C(=O)Nc2c(-c3ccccn3)ccnc2Cl)cn1]

To a solution of N-(2-chloro-4-iodo-3-pyridyl)-2-isopropyl-pyrimidine-5-carboxamide (0.15 g, 0.36 mmol) in THF (2.0 mL) were added tetrakis(triphenylphosphine)palladium(0) (0.042 g, 0.036 mmol) and 2-pyridylzinc bromide (1.4 mL, 0.72 mmol). The mixture was stirred under nitrogen at 70° C. for 4h. After cooling to ambient temperature, the reaction mixture was diluted with ethyl acetate and washed with 1M HCl (20 mL) and water (20 mL), then concentrated in vacuo. Purification by column chromatography, eluting with a gradient of 5 to 100% ethyl acetate in heptane afforded the title compound as a yellow solid (0.10 g, 80% yield): MS (ES+) m/z 354.0 (M+1), 356.0 (M+1).

### Step 2. Preparation of N-(2′-(4,4-difluorocyclohex-1-en-1-yl)-[2,4′-bipyridin]-3′-yl)-2-isopropylpyrimidine-5-carboxamide

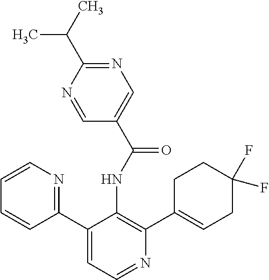 [SMILES: CC(C)c1ncc(C(=O)Nc2c(-c3ccccn3)ccnc2C2=CCC(F)(F)CC2)cn1]

A mixture of N-(2′-chloro-[2,4′-bipyridin]-3′-yl)-2-isopropylpyrimidine-5-carboxamide (0.10 g, 0.29 mmol) in dioxane (1.3 mL) and water (0.14 mL) was degassed with nitrogen for 10 minutes. To the reaction mixture was added potassium carbonate (0.12 g, 0.86 mmol), 2-(4,4-difluorocyclohex-1-en-1-yl)-4,4,5,5-tetramethyl-1,3,2-dioxaborolane (0.11 g, 0.43 mmol), [1,1′-Bis(diphenylphosphino)ferrocene]dichloropalladium(II), complex with dichloromethane (1:1) (0.024 g, 0.029 mmol), and the reaction mixture was stirred at 100° C. for 3 h. After cooling to ambient temperature, the mixture was filtered through a bed of Celite, and the filter pad was washed with ethyl acetate (2×20 mL). The filtrate was washed with saturated ammonium chloride (2×25 mL) and concentrated in vacuo. Purification of the residue by preparative reverse-phase HPLC, eluting with 10-90% acetonitrile in water containing 0.5% formic acid, afforded the title compound as a colorless solid (0.033 g, 25% yield): ¹H-NMR (400 MHz; DMSO-d₆) δ 8.96 (s, 2H), 8.67 (ddd, J=4.9, 1.7, 0.9 Hz, 1H), 8.61 (d, J=5.1 Hz, 1H), 7.90 (td, J=7.8, 1.8 Hz, 1H), 7.70-7.68 (m, 1H), 7.61 (d, J=5.1 Hz, 1H), 7.43 (ddd, J=7.6, 4.9, 1.1 Hz, 1H), 5.79 (m, 1H), 3.23 (dq, J=13.8, 6.9 Hz, 1H), 2.77 (m, 2H), 2.62-2.54 (m, 2H), 2.15 (tt, J=13.7, 6.8 Hz, 2H), 1.34 (d, J=6.9 Hz, 6H); MS (ES+) m/z 436.2 (M+1).

### Example 388

### Synthesis of N-(2-(4,4-difluorocyclohexyl)-4-methoxypyridin-3-yl)-2-isopropylpyrimidine-5-carboxamide 2

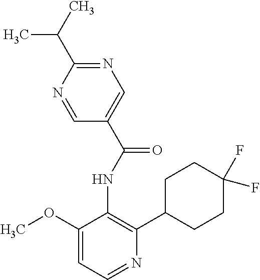 [SMILES: COc1ccnc(C2CCC(F)(F)CC2)c1NC(=O)c1cnc(C(C)C)nc1]

### Step 1. Preparation of N-(2-chloro-4-methoxypyridin-3-yl)-2-isopropylpyrimidine-5-carboxamide

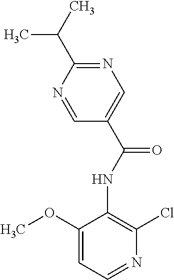 [SMILES: COc1ccnc(Cl)c1NC(=O)c1cnc(C(C)C)nc1]

A vial containing 2-chloro-4-methoxy-pyridin-3-ylamine (0.25 g, 1.6 mmol), 2-chloro-1-methylpyridinium iodide (1.2 g, 4.7 mmol), 2-isopropylpyrimidine-5-carboxylic acid (0.52 g, 3.2 mmol), and anhydrous tetrahydrofuran (7.8 mL) was charged with N,N-diisopropylethylamine (2.7 mL, 16 mmol). The reaction vessel was sealed and stirred at 65° C. for 20 h. The reaction mixture was diluted with ethyl acetate (20 mL). The organic layer was washed with 1M sodium hydroxide (20 mL), saturated ammonium chloride (2×30 mL), and water (30 mL), then dried over anhydrous magnesium sulfate, filtered, and concentrated in vacuo. The residue was purified by column chromatography, eluting with a gradient of 5 to 80% of ethyl acetate in heptane, to provide the title compound as a light-yellow solid (0.25 g, 55% yield): MS (ES+) m/z 307.0 (M+1), 309.0 (M+1).

### Step 2. Preparation of N-(2-(4,4-difluorocyclohexyl)-4-methoxypyridin-3-yl)-2-isopropylpyrimidine-5-carboxamide

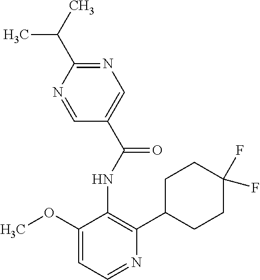 [SMILES: COc1ccnc(C2CCC(F)(F)CC2)c1NC(=O)c1cnc(C(C)C)nc1]

To a mixture of (4,4″-di-t-butyl-2,2″-bipyridine)bis[3,5-difluoro-2-[5-trifluoromethyl-2-pyridinyl kN)phenyl-kC]iridium(III) hexafluorophosphate (0.0088 g, 0.0078 mmol), dichloro(dimethoxyethane)nickel (0.017 g, 0.079 mmol), 4,4′-di-tert-butyl-2,2′-bipyridine (0.032 g, 0.12 mmol), N-(2-chloro-4-methoxypyridin-3-yl)-2-isopropylpyrimidine-5-carboxamide (0.24 g, 0.79 mmol), cesium carbonate (0.51 g, 1.6 mmol) and 4,4-difluorocyclohexanecarboxylic acid (0.26 g, 1.6 mmol) was added N,N-dimethylformamide (20 mL). The mixture was purged with nitrogen for 10 s and sealed. The reaction mixture was then stirred in front of Kessil PR160L lights (440 nm) for 20 h. The reaction mixture was quenched with saturated aqueous sodium bicarbonate solution (10 mL) and extracted with ethyl acetate (3×20 mL). The combined organic extracts were washed with water (3×50 mL) and brine (50 mL), dried over anhydrous sodium sulfate, and concentrated in vacuo. Purification of the residue by reverse-phase column chromatography, eluting with 5 to 95% acetonitrile in water containing 0.5% formic acid, afforded the title compound as a colorless solid (0.27 g, 85% yield): ¹H-NMR (400 MHz; DMSO-d₆) δ 10.04 (s, 1H), 9.24 (s, 2H), 8.41 (d, J=5.6 Hz, 1H), 7.05 (d, J=5.7 Hz, 1H), 3.83 (s, 3H), 3.25 (tt, J=13.8, 6.9 Hz, 1H), 2.04-1.77 (m, 9H), 1.33 (d, J=6.9 Hz, 6H); MS (ESI+) m/z 391.2 (M+1).

### Example 389

### Synthesis of N-(4-(cyclopropyl(methyl)amino)-2-(4,4-difluorocyclohexyl)pyridin-3-yl)-2-isopropylpyrimidine-5-carboxamide

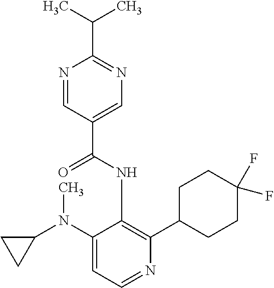 [SMILES: CC(C)c1ncc(C(=O)Nc2c(N(C)C3CC3)ccnc2C2CCC(F)(F)CC2)cn1]

### Step 1. Preparation of 2-chloro-N-cyclopropyl-N-methyl-3-nitropyridin-4-amine

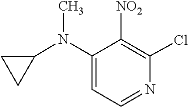

To a mixture of 2,4-dichloro-3-nitropyridine (1.0 g, 5.2 mmol), N-methylcyclopropanamine (0.41 g, 5.7 mmol) in anhydrous DMSO (10 mL) was added N,N-diisopropylethylamine (6.2 mL, 5.2 mmol) and was stirred for 2 h at ambient temperature. The reaction mixture was diluted with water (20 mL) and extracted with ethyl acetate (2×20 mL). The organic layer was washed with brine (20 mL), dried over anhydrous magnesium sulfate, filtered, and concentrated in vacuo. The residue was purified by column chromatography, eluting with a gradient of 5 to 60% of ethyl acetate in heptane, to provide the title compound as a yellow oil (0.60 g, 51% yield): MS (ES+) m/z 228.0 (M+1), 229.9 (M+1).

### Step 2. Preparation of N-cyclopropyl-2-(4,4-difluorocyclohex-1-en-1-yl)-N-methyl-3-nitropyridin-4-amine

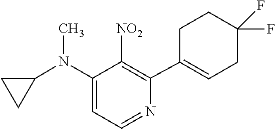 [SMILES: CN(c1ccnc(C2=CCC(F)(F)CC2)c1[N+](=O)[O-])C1CC1]

A mixture of 2-chloro-N-cyclopropyl-N-methyl-3-nitropyridin-4-amine (0.31 g, 1.3 mmol) in 1,4-dioxane (8.6 mL) and water (0.96 mL) was degassed with nitrogen for 10 minutes. To the reaction mixture was added potassium carbonate (0.56 g, 4.0 mmol), 2-(3,4-dihydro-2H-pyran-6-yl)-4,4,5,5-tetramethyl-1,3,2-dioxaborolane (0.29 g, 1.2 mmol), [1,1′-bis(diphenylphosphino)ferrocene]dichloropalladium(II), complex with dichloromethane (1:1) (0.11 g, 0.13 mmol), and the reaction mixture was stirred at 90° C. for 3 h. After cooling to ambient temperature, the mixture was filtered through a bed of Celite, and the filter pad was washed with ethyl acetate (2×20 mL). The filtrate was washed with saturated ammonium chloride (2×25 mL) and concentrated in vacuo. The resulting residue was used as is in the next step (0.41 g, quant. yield, crude): MS (ES+) m/z 310.2 (M+1).

### Step 3. Preparation of N⁴-cyclopropyl-2-(4,4-difluorocyclohexyl)-N⁴-methylpyridine-3,4-diamine

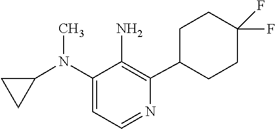 [SMILES: CN(c1ccnc(C2CCC(F)(F)CC2)c1N)C1CC1]

To a solution of 2-chloro-N-cyclopropyl-N-methyl-3-nitropyridin-4-amine (0.42 g, 1.4 mmol) in methanol (14 mL) and ethyl acetate (14 mL) was added palladium on activated carbon (0.16 g, 1.5 mmol, 10% purity) under a nitrogen atmosphere. The mixture was stirred at ambient temperature for 12 h under hydrogen (15 psi). The reaction mixture was filtered, and the filtrate was evaporated under reduced pressure to afford the title compound as an off-white solid (0.10 g, 27% yield): MS (ES+) m/z 282.2 (M+1).

### Step 4. Preparation of N-(4-(cyclopropyl(methyl)amino)-2-(4,4-difluorocyclohexyl)pyridin-3-yl)-2-isopropylpyrimidine-5-carboxamide

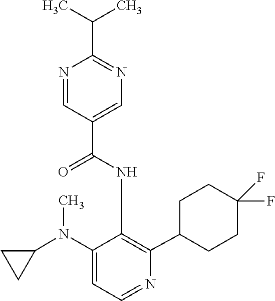 [SMILES: CC(C)c1ncc(C(=O)Nc2c(N(C)C3CC3)ccnc2C2CCC(F)(F)CC2)cn1]

A mixture containing N⁴-cyclopropyl-2-(4,4-difluorocyclohexyl)-N⁴-methylpyridine-3,4-diamine (0.094 g, 0.33 mmol), 2-chloro-1-methylpyridinium iodide (0.26 g, 1.0 mmol), 2-Isopropylpyrimidine-5-carboxylic acid (0.083 g, 0.50 mmol), and anhydrous tetrahydrofuran (3.4 mL) was charged with N,N-diisopropylethylamine (0.35 mL, 2.0 mmol). The reaction vessel was sealed and stirred at 68° C. for 20 h. The reaction mixture was diluted with ethyl acetate (20 mL). The organic layer was washed with 1M sodium hydroxide (20 mL), saturated ammonium chloride (2×30 mL), and water (30 mL), then dried over anhydrous magnesium sulfate, filtered, and concentrated in vacuo. Purification of the residue by preparative reverse-phase HPLC, eluting with 10-50% acetonitrile in water containing 0.5% formic acid, afforded the title compound as a colorless solid (0.10 g, 74% yield): ¹H-NMR (400 MHz; DMSO-d₆) S 10.02 (s, 1H), 9.23 (s, 2H), 8.19 (d, J=5.6 Hz, 1H), 6.98 (d, J=5.7 Hz, 1H), 3.25 (ddddd, J=10.8, 6.5, 4.7, 3.9, 1.3 Hz, 1H), 2.99-2.92 (m, 1H), 2.87 (s, 3H), 2.61 (tt, J=6.6, 3.3 Hz, 1H), 2.08-1.84 (m, 8H), 1.32 (d, J=6.9 Hz, 6H), 0.75-0.71 (m, 2H), 0.42-0.38 (m, 2H); MS (ES+) m/z 430.2 (M+1).

### Example 390

### Synthesis of N-(2′-(4,4-difluorocyclohexyl)-3-fluoro-[2,4′-bipyridin]-3′-yl)-2-isopropylpyrimidine-5-carboxamide

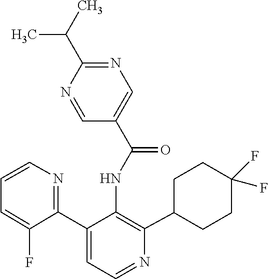 [SMILES: CC(C)c1ncc(C(=O)Nc2c(-c3ncccc3F)ccnc2C2CCC(F)(F)CC2)cn1]

### Step 1. Preparation of N-(2′-chloro-3-fluoro-[2,4′-bipyridin]-3′-yl)-2-isopropylpyrimidine-5-carboxamide

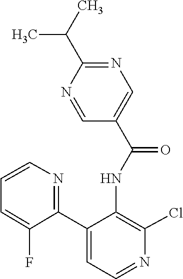 [SMILES: CC(C)c1ncc(C(=O)Nc2c(-c3ncccc3F)ccnc2Cl)cn1]

To a solution of 3-fluoro-2-iodopyridine (0.085 g, 0.38 mmol) in THF (3.0 mL) was added isopropylmagnesium chloride (2M in THF) (0.19 mL, 0.38 mmol) at −40° C. and the mixture was stirred for 20 minutes. To this solution was added zinc chloride (1M in THF) (1.5 mL, 1.5 mmol) in one portion at −40° C. The reaction mixture was allowed to warm to ambient temperature and stirred for another 1.5 h. A solution of N-(2-chloro-4-iodo-3-pyridyl)-2-isopropyl-pyrimidine-5-carboxamide (0.15 g, 0.38 mmol) in THF (3.0 mL) and tetrakis(triphenylphosphine)palladium(0) (0.044 g, 0.038 mmol) was added, and the reaction mixture was held at reflux for 1 h. The reaction mixture was partitioned between ethyl acetate (20 mL) and 10% aqueous NaHCO₃ solution (20 mL). The organic layer was washed with water (30 mL). The combined organic layers were dried over anhydrous magnesium sulfate, filtered, and concentrated in vacuo. Purification by column chromatography, eluting with a gradient of 5 to 100% ethyl acetate in heptane afforded the title compound as an off-white solid (0.087 g, 61% yield): MS (ES+) m/z 372.0 (M+1), 374.0 (M+1).

### Step 2. Preparation of N-(2′-(4,4-difluorocyclohexyl)-3-fluoro-[2,4′-bipyridin]-3′-yl)-2-isopropylpyrimidine-5-carboxamide

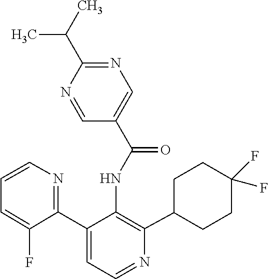 [SMILES: CC(C)c1ncc(C(=O)Nc2c(-c3ncccc3F)ccnc2C2CCC(F)(F)CC2)cn1]

To a mixture of (4,4″-di-t-butyl-2,2″-bipyridine)bis[3,5-difluoro-2-[5-trifluoromethyl-2-pyridinyl kN)phenyl-kC]iridium(III) hexafluorophosphate (0.0022 g, 0.0020 mmol), dichloro(dimethoxyethane)nickel (0.0044 g, 0.020 mmol), 4,4′-di-tert-butyl-2,2′-bipyridine (0.0080 g, 0.030 mmol), N-(2′-chloro-3-fluoro-[2,4′-bipyridin]-3′-yl)-2-isopropylpyrimidine-5-carboxamide (0.074 g, 0.20 mmol), cesium carbonate (0.13 g, 0.40 mmol) and 4,4-difluorocyclohexanecarboxylic acid (0.065 g, 0.40 mmol) was added N,N-dimethylformamide (5.0 mL). The mixture was purged with nitrogen for 10 s and sealed. The reaction mixture was then stirred in front of Kessil PR160L lights (440 nm) for 20 h. The reaction mixture was quenched with saturated aqueous sodium bicarbonate solution (10 mL) and extracted with ethyl acetate (3×20 mL). The combined organic extracts were washed with water (3×50 mL) and brine (50 mL), dried over anhydrous sodium sulfate, and concentrated in vacuo. Purification of the residue by preparative reverse-phase HPLC, eluting with 10 to 90% acetonitrile in water containing 0.5% formic acid afforded the title compound as a colorless solid (0.11 g, 53% yield): ¹H-NMR (400 MHz; DMSO-d₆) δ 10.40 (s, 1H), 8.95 (s, 2H), 8.66 (d, J=4.9 Hz, 1H), 8.47 (dt, J=4.6, 1.6 Hz, 1H), 7.84 (ddd, J=10.2, 8.5, 1.3 Hz, 1H), 7.66-7.53 (m, 2H), 3.20 (dquintet, J=13.8, 6.9 Hz, 2H), 2.11-1.85 (m, 8H), 1.29 (d, J=6.9 Hz, 6H); MS (ES−) m/z 454.6 (M−1).

In a similar manner as described in examples disclosed herein, utilizing the appropriately substituted starting materials and intermediates, the following compounds were prepared:

| Example | Structure | Amount | | |
|---|---|---|---|---|
| No. | Name | Yield | MS (ES+) m/z | ¹H NMR |
| 391 | 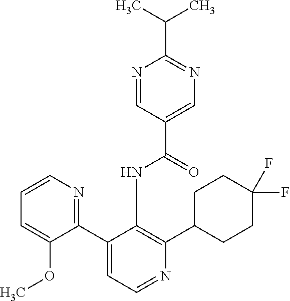 [SMILES: COc1cccnc1-c1ccnc(C2CCC(F)(F)CC2)c1NC(=O)c1cnc(C(C)C)nc1] N-(2′-(4,4- difluorocyclohexyl)-3- methoxy-[2,4′-bipyridin]- 3′-yl)-2-isopropylpyrimidine-5- carboxamide | 0.0094 g 24% | 468.2 (M + 1) | (400 MHz, DMSO-d₆) δ 10.13 (s, 1H), 8.92 (s, 2H), 8.58 (d, J = 4.9 Hz, 1H), 8.19 (dd, J = 4.6, 0.9 Hz, 1H), 7.54- 7.51 (m, 1H), 7.40-7.35 (m, 2H), 3.70 (s, 3H), 3.20 (dq, J = 13.7, 6.9 Hz, 2H), 2.12-2.06 (m, 2H), 1.95-1.83 (m, 6H), 1.28 (d, J = 6.9 Hz, 6H) |

### Example 392

### Synthesis of N-(2-(4,4-difluorocyclohexyl)-4-(3,4-dihydro-2H-pyran-6-yl)pyridin-3-yl)-2-isopropoxypyrimidine-5-carboxamide

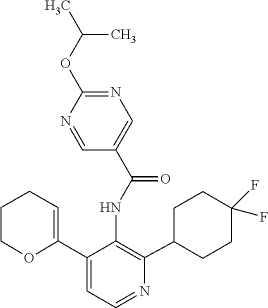 [SMILES: CC(C)Oc1ncc(C(=O)Nc2c(C3=CCCCO3)ccnc2C2CCC(F)(F)CC2)cn1]

### Step 1. Preparation of 2-chloro-4-(3,4-dihydro-2H-pyran-6-yl)pyridin-3-amine

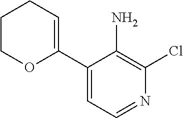 [SMILES: Nc1c(C2=CCCCO2)ccnc1Cl]

A mixture of 2-chloro-4-iodo-pyridin-3-amine (0.20 g, 0.79 mmol) in 1,4-dioxane (3.0 mL) and water (0.30 mL) was degassed with nitrogen for 10 minutes. To the reaction mixture was added potassium carbonate (0.33 g, 2.4 mmol), 2-(3,4-dihydro-2H-pyran-6-yl)-4,4,5,5-tetramethyl-1,3,2-dioxaborolane (0.25 g, 1.2 mmol), [1,1′-bis(diphenylphosphino)ferrocene]dichloropalladium(II), complex with CH₂Cl₂ (1:1) (0.067 g, 0.079 mmol), and the reaction mixture was stirred at 90° C. for 3 h. After cooling to ambient temperature, the mixture was filtered through a bed of Celite, and the filter pad was washed with ethyl acetate (2×20 mL). The combined filtrate was washed with saturated ammonium chloride (2×25 mL) and concentrated in vacuo. The residue was purified by column chromatography, eluting with a gradient of 5 to 100% ethyl acetate in heptane, to afford the title compound as a yellow solid (0.11 g, 64% yield): MS (ES+) m/z 211.0 (M+1), 213.0 (M+1).

### Step 2. Preparation of 2-chloro-N-(2-chloro-4-(3,4-dihydro-2H-pyran-6-yl)pyridin-3-yl)pyrimidine-5-carboxamide and 2-chloro-N-(2-chloro-4-(3,4-dihydro-2H-pyran-6-yl)pyridin-3-yl)-N-(2-chloropyrimidine-5-carbonyl)pyrimidine-5-carboxamide

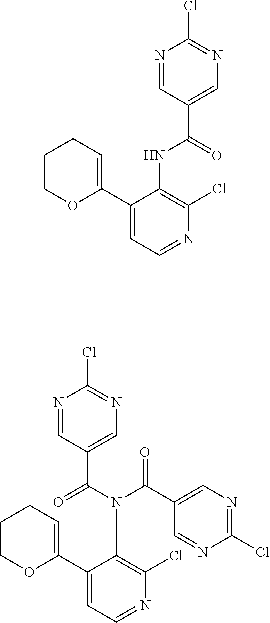 [SMILES: O=C(Nc1c(C2=CCCCO2)ccnc1Cl)c1cnc(Cl)nc1 ; O=C(c1cnc(Cl)nc1)N(C(=O)c1cnc(Cl)nc1)c1c(C2=CCCCO2)ccnc1Cl]

To a mixture of 2-chloro-4-(3,4-dihydro-2H-pyran-6-yl)pyridin-3-amine (0.11 g, 0.50 mmol), 2-chloropyrimidine-5-carboxylic acid (0.12 g, 0.75 mmol), and 2-chloro-1-methylpyridinium iodide (0.26 g, 1.0 mmol) and N,N-diisopropylethylamine (0.50 mL, 2.9 mmol) was added anhydrous THF (3.4 mL). The reaction mixture was stirred at 65° C. for 20 h. After cooling to ambient temperature, the mixture was diluted with ethyl acetate (20 mL). The organic layer was washed with water (2×30 mL), dried over anhydrous magnesium sulfate, filtered, and concentrated in vacuo. Both mono- and bis-alkylated products were isolated. The resulting solid was used in the next step without further purification (0.11 g, 53% yield): MS1 (ES+) m/z 351.2 (M+1), 353.2 (M+1); MS2 (ES+) m/z 493.0 (M+1), 495.0 (M+1).

### Step 3. Preparation of N-(2-chloro-4-(3,4-dihydro-2H-pyran-6-yl)pyridin-3-yl)-2-isopropoxypyrimidine-5-carboxamide

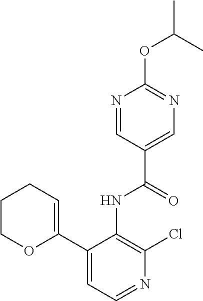 [SMILES: CC(C)Oc1ncc(C(=O)Nc2c(C3=CCCCO3)ccnc2Cl)cn1]

To a crude solid of 2-chloro-N-(2-chloro-4-(3,4-dihydro-2H-pyran-6-yl)pyridin-3-yl)pyrimidine-5-carboxamide and 2-chloro-N-(2-chloro-4-(3,4-dihydro-2H-pyran-6-yl)pyridin-3-yl)-N-(2-chloropyrimidine-5-carbonyl)pyrimidine-5-carboxamide (0.11 g, 0.22 mmol) in anhydrous DMF (1.1 mL) was added 2-propanol (2.8 mL, 2.2 mmol) and 60% sodium hydride dispersion in mineral oil (0.043 g, 1.8 mmol). The solution was stirred at 40° C. for 1 h. The reaction mixture was diluted with ethyl acetate (10 mL) and washed with saturated ammonium chloride solution (2×20 mL). The organic layer was dried over anhydrous magnesium sulfate, filtered, and concentrated in vacuo. Purification of the residue by column chromatography, eluting with 5 to 70% ethyl acetate in heptane, provided the title compound as a colorless solid (0.059 g, 71% yield): MS (ES+) m/z 375.0 (M+1), 377.0 (M+1).

### Step 4. Preparation of N-(2-(4,4-difluorocyclohexyl)-4-(3,4-dihydro-2H-pyran-6-yl)pyridin-3-yl)-2-isopropoxypyrimidine-5-carboxamide

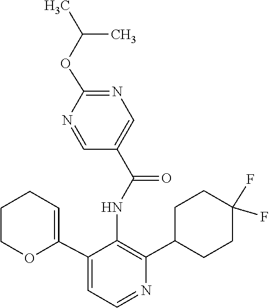 [SMILES: CC(C)Oc1ncc(C(=O)Nc2c(C3=CCCCO3)ccnc2C2CCC(F)(F)CC2)cn1]

To a mixture of (4,4″-di-t-butyl-2,2″-bipyridine)bis[3,5-difluoro-2-[5-trifluoromethyl-2-pyridinyl-kN)phenyl-kC]iridium(II) hexafluorophosphate (0.0015 g, 0.0013 mmol), dichloro(dimethoxyethane)nickel (0.0029 g, 0.013 mmol), 4,4′-di-tert-butyl-2,2′-bipyridine (0.0054 g, 0.020 mmol), N-(2-chloro-4-(3,4-dihydro-2H-pyran-5-yl)pyridin-3-yl)-2-isopropylpyrimidine-5-carboxamide (0.050 g, 0.13 mmol), cesium carbonate (0.087 g, 0.27 mmol) and 4,4-difluorocyclohexanecarboxylic acid (0.044 g, 0.27 mmol) was added N,N-dimethylformamide (3.3 mL). The mixture was purged with nitrogen for 10 s and sealed. The reaction mixture was then stirred in front of Kessil PR160L lights (440 nm) for 20 h. The reaction mixture was quenched with saturated aqueous sodium bicarbonate solution (10 mL) and extracted with ethyl acetate (3×20 mL). The combined organic extracts were washed with water (3×50 mL) and brine (50 mL), dried over anhydrous sodium sulfate, and concentrated in vacuo. Purification of the residue by preparative reverse-phase HPLC, eluting with a gradient of 15-90% acetonitrile in water containing 0.5% formic acid, afforded the title compound as a colorless solid (0.017 g, 26% yield): ¹H-NMR (400 MHz; DMSO-d₆) δ 10.05 (s, 1H), 9.10 (s, 2H), 8.47 (d, J=5.0 Hz, 1H), 7.28-7.26 (m, 1H), 5.32 (quintet, J=6.2 Hz, 1H), 5.18 (t, J=4.0 Hz, 1H), 3.94 (dd, J=5.8, 4.2 Hz, 2H), 3.14-3.06 (m, 1H), 2.09-2.04 (m, 4H), 1.93-1.73 (m, 8H), 1.37 (d, J=6.2 Hz, 6H); MS (ES+) m/z 459.6 (M+1)

In a similar manner as described in examples disclosed herein, utilizing the appropriately substituted starting materials and intermediates, the following compounds were prepared:

| Example | | Amount | | |
|---|---|---|---|---|
| No. | Structure | Yield | MS (ES+) m/z | ¹H NMR |
| 393 | 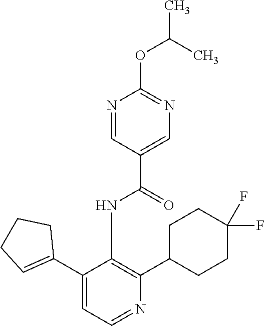 [SMILES: CC(C)Oc1ncc(C(=O)Nc2c(C3=CCCC3)ccnc2C2CCC(F)(F)CC2)cn1] N-(4-(cyclopent-1-en-1- yl)-2-(4,4-difluorocyclohexyl) pyridin-3-yl)-2- isopropoxypyrimidine-5- carboxamide | 0.0072 g 14% | 443.6 (M + 1) | (400 MHz, DMSO-d₆) δ 10.19 (s, 1H), 9.10 (s, 2H), 8.44 (d, J = 5.0 Hz, 1H), 7.26 (d, J = 5.0 Hz, 1H), 6.19 (q, J = 2.1 Hz, 1H), 5.32 (dt, J = 12.4, 6.2 Hz, 1H), 3.12-3.03 (m, 1H), 2.63-2.59 (m, 2H), 2.38 (td, J = 7.3, 2.2 Hz, 2H), 2.09-2.02 (m, 2H), 1.96-1.77 (m, 8H), 1.37 (d, J = 6.2 Hz, 6H) |

### Example 394

### Synthesis of N-(2-(4,4-difluorocyclohexyl)-4-(3,4-dihydro-2H-pyran-5-yl)pyridin-3-yl)-2-isopropylpyrimidine-5-carboxamide

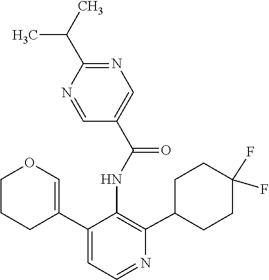 [SMILES: CC(C)c1ncc(C(=O)Nc2c(C3=COCCC3)ccnc2C2CCC(F)(F)CC2)cn1]

### Step 1. Preparation of N-(2-chloro-4-(3,4-dihydro-2H-pyran-5-yl)pyridin-3-yl)-2-isopropylpyrimidine-5-carboxamide

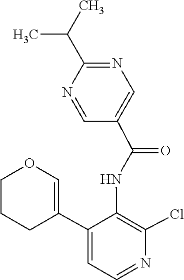 [SMILES: CC(C)c1ncc(C(=O)Nc2c(C3=COCCC3)ccnc2Cl)cn1]

A mixture of N-(2-chloro-4-iodopyridin-3-yl)-2-isopropylpyrimidine-5-carboxamide (0.15 g, 0.37 mmol) in dioxane (2.4 mL) and water (0.27 mL) was degassed with nitrogen for 10 minutes. To the reaction mixture was added potassium carbonate (0.15 g, 1.1 mmol), 2-(3,4-dihydro-2H-pyran-5-yl)-4,4,5,5-tetramethyl-1,3,2-dioxaborolane (0.12 g, 0.56 mmol), [1,1′-bis(diphenylphosphino)ferrocene]dichloropalladium(II), complex with dichloromethane (1:1) (0.030 g, 0.037 mmol), and the reaction mixture was stirred at 90° C. for 3 h. After cooling to ambient temperature, the mixture was filtered through a bed of Celite, and the filter pad was washed with ethyl acetate (2×20 mL). The filtrate was washed with saturated ammonium chloride (2×25 mL) and concentrated in vacuo. The residue was purified by column chromatography, eluting with a gradient of 5 to 100% ethyl acetate in heptane, to afford the title compound as a yellow solid (0.11 g, 80% yield): MS (ES+) m/z 359.0 (M+1), 361.0 (M+1).

### Step 2. Preparation of N-(2-(4,4-difluorocyclohexyl)-4-(3,4-dihydro-2H-pyran-5-yl)pyridin-3-yl)-2-isopropylpyrimidine-5-carboxamide

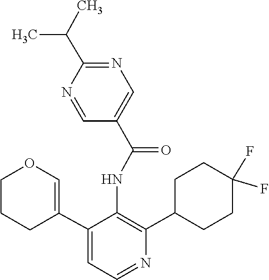 [SMILES: CC(C)c1ncc(C(=O)Nc2c(C3=COCCC3)ccnc2C2CCC(F)(F)CC2)cn1]

To a mixture of (4,4″-di-t-butyl-2,2″-bipyridine)bis[3,5-difluoro-2-[5-trifluoromethyl-2-pyridinyl kN)phenyl-kC]iridium(III) hexafluorophosphate (0.0036 g, 0.0032 mmol), dichloro(dimethoxyethane)nickel (0.0071 g, 0.032 mmol), 4,4′-di-tert-butyl-2,2′-bipyridine (0.013 g, 0.049 mmol), N-(2-chloro-4-(3,4-dihydro-2H-pyran-5-yl)pyridin-3-yl)-2-isopropylpyrimidine-5-carboxamide (0.12 g, 0.32 mmol), cesium carbonate (0.21 g, 0.65 mmol) and 4,4-difluorocyclohexanecarboxylic acid (0.11 g, 0.65 mmol) was added N,N-dimethylformamide (8.1 mL). The mixture was purged with nitrogen for 10 s and sealed. The reaction mixture was then stirred in front of Kessil PR160L lights (440 nm) for 20 h. The reaction mixture was quenched with saturated aqueous sodium bicarbonate solution (10 mL) and extracted with ethyl acetate (3×20 mL). The combined organic extracts were washed with water (3×50 mL) and brine (50 mL), dried over anhydrous sodium sulfate, and concentrated in vacuo. Purification of the residue by preparative reverse-phase HPLC, eluting with a gradient of 10 to 80% acetonitrile in water containing 0.5% formic acid, afforded the title compound as a colorless solid (0.11 g, 53% yield): ¹H-NMR (400 MHz; DMSO-d₆) δ 9.11 (s, 2H), 8.47 (d, J=4.7 Hz, 1H), 7.35 (d, J=4.5 Hz, 1H), 5.56 (d, J=4.4 Hz, 1H), 3.74 (d, J=11.1 Hz, 1H), 3.48-3.45 (m, 1H), 3.26 (tt, J=13.8, 6.9 Hz, 1H), 2.85-2.80 (m, 1H), 2.36-1.41 (m, 12H), 1.34 (d, J=6.9 Hz, 6H); MS (ES+) m/z 443.2 (M+1).

### Example 395

### Synthesis of N-(4-(5-chloro-2-fluorophenyl)-2-(4,4-difluorocyclohexyl)pyridin-3-yl)-2-isopropylpyrimidine-5-carboxamide

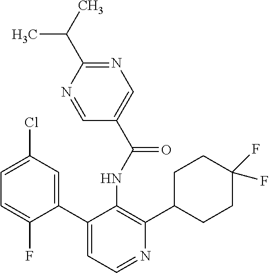 [SMILES: CC(C)c1ncc(C(=O)Nc2c(-c3cc(Cl)ccc3F)ccnc2C2CCC(F)(F)CC2)cn1]

### Step 1. Preparation of N-(2-chloro-4-(5-chloro-2-fluorophenyl)pyridin-3-yl)-2-isopropylpyrimidine-5-carboxamide

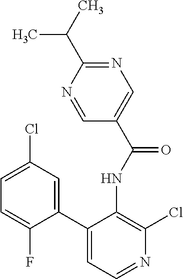 [SMILES: CC(C)c1ncc(C(=O)Nc2c(-c3cc(Cl)ccc3F)ccnc2Cl)cn1]

A mixture of N-(2-chloro-4-iodopyridin-3-yl)-2-isopropylpyrimidine-5-carboxamide (0.15 g, 0.37 mmol) in dioxane (2.8 mL) and water (0.30 mL) was degassed with nitrogen for 10 minutes. To the reaction mixture was added potassium carbonate (0.15 g, 1.1 mmol), 5-chloro-2-fluorophenylboronic acid (0.097 g, 0.56 mmol), [1,1′-bis(diphenylphosphino)ferrocene]dichloropalladium(II), complex with dichloromethane (1:1) (0.030 g, 0.037 mmol), and the reaction mixture was stirred at 90° C. for 3 h. After cooling to ambient temperature, the mixture was filtered through a bed of Celite, and the filter pad was washed with ethyl acetate (2×20 mL). The filtrate was washed with saturated ammonium chloride (2×25 mL) and concentrated in vacuo. The residue was purified by column chromatography, eluting with a gradient of 5 to 50% ethyl acetate in heptane, to afford the title compound as a yellow solid (0.14 g, 89% yield): MS (ES+) m/z 405.0 (M+1), 407.0 (M+1).

### Step 2. Preparation of N-(4-(5-chloro-2-fluorophenyl)-2-(4,4-difluorocyclohex-1-en-1-yl)pyridin-3-yl)-2-isopropylpyrimidine-5-carboxamide

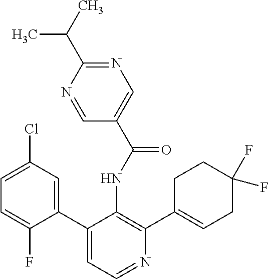 [SMILES: CC(C)c1ncc(C(=O)Nc2c(-c3cc(Cl)ccc3F)ccnc2C2=CCC(F)(F)CC2)cn1]

A mixture of N-(2-chloro-4-iodopyridin-3-yl)-2-isopropylpyrimidine-5-carboxamide (0.11 g, 0.27 mmol) in dioxane (2.0 mL) and water (0.22 mL) was degassed with nitrogen for 10 minutes. To the reaction mixture was added potassium carbonate (0.11 g, 0.81 mmol), 2-(4,4-difluorocyclohex-1-en-1-yl)-4,4,5,5-tetramethyl-1,3,2-dioxaborolane (0.097 g, 0.56 mmol), [1,1′-bis(diphenylphosphino)ferrocene]dichloropalladium(II), complex with dichloromethane (1:1) (0.022 g, 0.037 mmol), and the reaction mixture was stirred at 90° C. for 3 h. After cooling to ambient temperature, the mixture was filtered through a bed of Celite and the filter pad was washed with ethyl acetate (2×20 mL). The combined filtrate was washed with saturated ammonium chloride (2×25 mL) and concentrated in vacuo. The resulting solid was used in the next step without further purification (0.17 g, quant. yield, crude): MS (ES+) m/z 487.2 (M+1), 489.2 (M+1).

### Step 3. Preparation of N-(4-(5-chloro-2-fluorophenyl)-2-(4,4-difluorocyclohexyl)pyridin-3-yl)-2-isopropylpyrimidine-5-carboxamide

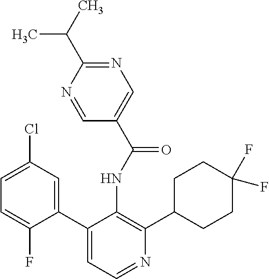 [SMILES: CC(C)c1ncc(C(=O)Nc2c(-c3cc(Cl)ccc3F)ccnc2C2CCC(F)(F)CC2)cn1]

To a solution of N-(4-(5-chloro-2-fluorophenyl)-2-(4,4-difluorocyclohex-1-en-1-yl)pyridin-3-yl)-2-isopropylpyrimidine-5-carboxamide (0.13 g, 0.27 mmol) in methanol (3.4 mL) and ethyl acetate (3.4 mL) was added palladium on activated carbon (0.043 g, 0.40 mmol, 10% purity) under a nitrogen atmosphere. The mixture was stirred at ambient temperature for 12 h under hydrogen (15 psi). The reaction mixture was filtered and the filtrate was evaporated under reduced pressure to afford the title compound as a colorless solid (0.013 g, 9.7% yield):H-NMR (400 MHz; DMSO-d₆) d 10.43 (s, 1H), 8.97 (s, 2H), 8.63 (d, J=4.9 Hz, 1H), 7.50-7.47 (m, 2H), 7.38 (dd, J=16.1, 6.9 Hz, 2H), 3.21 (dquintet, J=13.6, 6.8 Hz, 2H), 2.11-1.81 (, 8H), 1.29 (d, J=6.9 Hz, 6H); MS (ES+) m/z 489.2 (M+1), 491.0 (M+1).

In a similar manner as described in examples disclosed herein, utilizing the appropriately substituted starting materials and intermediates, the following compounds were prepared:

| Example | Structure | Amount | | |
|---|---|---|---|---|
| No. | Name | Yield | MS (ES+) m/z | ¹H NMR |
| 396 | 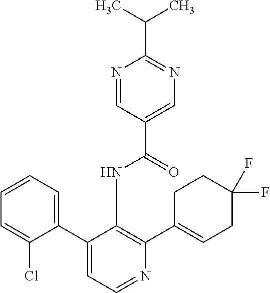 [SMILES: CC(C)c1ncc(C(=O)Nc2c(-c3ccccc3Cl)ccnc2C2=CCC(F)(F)CC2)cn1] N-(4-(2-chlorophenyl)-2- (4,4-difluorocyclohex-1- en-1-yl)pyridin-3-yl)-2- isopropylpyrimidine-5- carboxamide | 0.055 g 45% | 469.0 (M + 1), 471.2 (M + 1) | (400 MHz, DMSO-d₆) δ 10.21 (s, 1H), 8.81- 8.79 (m, 2H), 8.60 (d, J = 4.9 Hz, 1H), 7.58-7.55 (m, 1H), 7.42-7.31 (m, 4H), 5.82 (s, 1H), 3.17 (7, J = 6.9 Hz, 1H), 2.71-2.69 (m, 2H), 2.65-2.57 (m, 2H), 2.19-2.08 (m, 2H), 1.27 (d, J = 6.9 Hz, 6H) |

In a similar manner as described in examples disclosed herein, utilizing the appropriately substituted starting materials and intermediates, the following compounds were prepared:

| Example | Structure | Amount | | |
|---|---|---|---|---|
| No. | Name | Yield | MS (ES+) m/z | ¹H NMR |
| 397 | 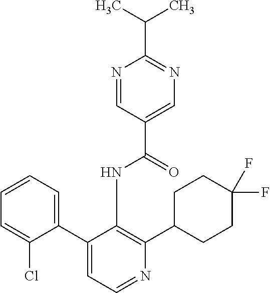 [SMILES: CC(C)c1ncc(C(=O)Nc2c(-c3ccccc3Cl)ccnc2C2CCC(F)(F)CC2)cn1] N-(4-(2-chlorophenyl)-2- (4,4-difluorocyclohexyl) pyridin-3-yl)-2- isopropylpyrimidine-5- carboxamide | 0.0057 g 10% | 471.0 (M + 1), 473.2 (M + 1) | (400 MHz, DMSO-d₆) δ 10.32 (s, 1H), 8.88 (s, 2H), 8.61 (d, J = 4.9 Hz, 1H), 7.55-7.53 (m, 1H), 7.40-7.29 (m, 4H), 3.23-3.14 (m, 2H), 2.11-1.82 (m, 8H), 1.27 (d, J = 6.9 Hz, 6H) |

### Example 398

### Synthesis of 2-(tert-butyl)-N-(2′-(4,4-difluorocyclohexyl)-3-fluoro-[2,4′-bipyridin]-3′-yl)pyrimidine-5-carboxamide

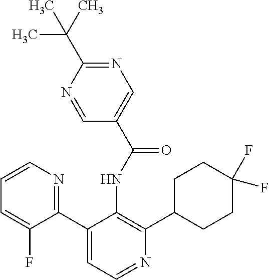 [SMILES: CC(C)(C)c1ncc(C(=O)Nc2c(-c3ncccc3F)ccnc2C2CCC(F)(F)CC2)cn1]

### Step 1. Preparation of tert-butyl (2′-chloro-3-fluoro-[2,4′-bipyridin]-3′-yl)carbamate

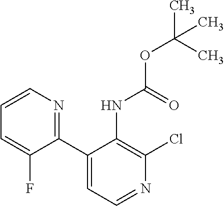 [SMILES: CC(C)(C)OC(=O)Nc1c(-c2ncccc2F)ccnc1Cl]

To a mixture of tert-butyl (2-chloro-4-iodopyridin-3-yl)carbamate (1.3 g, 3.7 mmol), 3-fluoro-2-(tributylstannyl)pyridine (1.6 g, 4.0 mmol), and anhydrous DMF (7.3 mL) were sparged with nitrogen gas for 5 min. To the sparged mixture was added copper(I) iodide (0.070 g, 0.37 mmol) and tetrakis(triphenylphosphine)palladium(0) (0.25 g, 0.22 mmol). The reaction mixture was heated to 115° C. for 3 h. After cooling to ambient temperature, the reaction mixture was diluted with ethyl acetate (100 mL). The organic layer was washed with saturated ammonium chloride (2×50 mL), dried over anhydrous magnesium sulfate, filtered, and concentrated in vacuo. The residue was purified by column chromatography, eluting with a gradient of 0 to 50% of ethyl acetate in heptane, to provide the title compound as a colorless solid (0.84 g, 71% yield): MS (ES+) m/z 324.2 (M+1), 326.2 (M+1).

### Step 2. Preparation of tert-butyl (2′-(4,4-difluorocyclohexyl)-3-fluoro-[2,4′-bipyridin]-3′-yl)carbamate

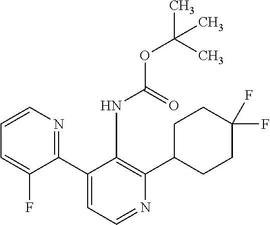 [SMILES: CC(C)(C)OC(=O)Nc1c(-c2ncccc2F)ccnc1C1CCC(F)(F)CC1]

To a mixture of (4,4″-di-t-butyl-2,2″-bipyridine)bis[3,5-difluoro-2-[5-trifluoromethyl-2-pyridinyl kN)phenyl-kC]iridium(III) hexafluorophosphate (0.029 g, 0.026 mmol), dichloro(dimethoxyethane)nickel (0.057 g, 0.26 mmol), 4,4′-di-tert-butyl-2,2′-bipyridine (0.11 g, 0.39 mmol), tert-butyl (2′-chloro-3-fluoro-[2,4′-bipyridin]-3′-yl)carbamate (0.84 g, 2.6 mmol), cesium carbonate (1.4 g, 4.2 mmol) and 4,4-difluorocyclohexanecarboxylic acid (0.64 g, 3.9 mmol) was added N,N-dimethylformamide (40 mL). The mixture was purged with nitrogen for 10 s and sealed. The reaction mixture was then stirred in front of Kessil PR160L lights (440 nm) for 8 h. The reaction mixture was diluted with ethyl acetate (300 mL). The organic layer was washed with saturated ammonium chloride (2×100 mL), dried over anhydrous magnesium sulfate, filtered, and concentrated in vacuo. The residue was purified by column chromatography, eluting with a gradient of 0 to 40% of ethyl acetate in heptane, to provide the title compound as a yellow solid (0.98 g, 92% yield): MS (ES+) m/z 408.4 (M+1).

### Step 3. Preparation of 2′-(4,4-difluorocyclohexyl)-3-fluoro-[2,4′-bipyridin]-3′-amine

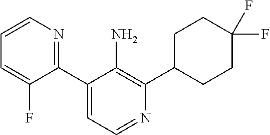 [SMILES: Nc1c(-c2ncccc2F)ccnc1C1CCC(F)(F)CC1]

A flask containing tert-butyl (2′-(4,4-difluorocyclohexyl)-3-fluoro-[2,4′-bipyridin]-3′-yl)carbamate (0.98 g, 2.4 mmol) was charged with 4M hydrogen chloride in 1,4-dioxane (12 mL). The reaction vessel was sealed and heated to 35° C. for 2 h. The heterogeneous reaction mixture was diluted with ethyl acetate (300 mL) and 5M sodium hydroxide (60 mL). The mixture was sonicated for 30 min. The organic layer was separated and washed with brine (100 mL), dried over anhydrous magnesium sulfate, filtered, and concentrated in vacuo. The residue was purified by column chromatography, eluting with a gradient of 10 to 50% of ethyl acetate in heptane, to provide the title compound as a yellow solid (0.41 g, 55% yield): ¹H-NMR (400 MHz; CDCl₃) δ 8.54 (dt, J=4.6, 1.4 Hz, 1H), 8.09 (d, J=5.1 Hz, 1H), 7.62 (ddd, J=10.5, 8.5, 1.5 Hz, 1H), 7.38 (ddd, J=8.4, 4.5, 3.9 Hz, 1H), 7.30-7.27 (m, 2H), 5.13 (s, 2H), 2.87-2.81 (m, 1H), 2.36-2.27 (m, 2H), 2.15-1.85 (m, 6H), MS (ES+) m/z 308.4 (M+1).

### Step 4. Preparation of 2-(tert-butyl)-N-(2′-(4,4-difluorocyclohexyl)-3-fluoro-[2,4′-bipyridin]-3′-yl)pyrimidine-5-carboxamide

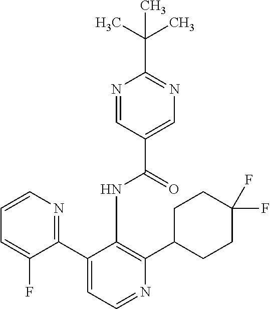 [SMILES: CC(C)(C)c1ncc(C(=O)Nc2c(-c3ncccc3F)ccnc2C2CCC(F)(F)CC2)cn1]

A vial containing 2′-(4,4-difluorocyclohexyl)-3-fluoro-[2,4′-bipyridin]-3′-amine (0.10 g, 0.32 mmol), 2-chloro-1-methylpyridinium iodide (0.25 g, 0.97 mmol), 2-tert-butylpyrimidine-5-carboxylic acid (0.088 g, 0.49 mmol), and anhydrous tetrahydrofuran (3.2 mL) was charged with N,N-diisopropylethylamine (0.34 mL, 1.9 mmol). The reaction vessel was sealed and stirred at 65° C. for 20 h. The reaction mixture was diluted with ethyl acetate (20 mL). The organic layer was washed with 1M sodium hydroxide (20 mL), saturated ammonium chloride (2×30 mL), and water (30 mL), then dried over anhydrous magnesium sulfate, filtered, and concentrated in vacuo. The residue was purified by column chromatography, eluting with a gradient of 5 to 100% of ethyl acetate in heptane, to provide the title compound as a light yellow solid (0.054 g, 35% yield): ¹H-NMR (400 MHz; DMSO-d₆) δ 10.39 (s, 1H), 8.96 (s, 2H), 8.66 (d, J=4.9 Hz, 1H), 8.48 (dt, J=4.6, 1.5 Hz, 1H), 7.85 (ddd, J=10.1, 8.6, 1.3 Hz, 1H), 7.51 (dt, J=8.6, 4.3 Hz, 1H), 7.47 (dd, J=4.9, 1.2 Hz, 1H), 3.24-3.19 (m, 1H), 2.13-2.06 (m, 2H), 2.02-1.86 (m, 6H), 1.37 (s, 9H); MS (ES+) m/z 470.2 (M+1).

In a similar manner as described in examples disclosed herein, utilizing the appropriately substituted starting materials and intermediates, the following compounds were prepared:

| Example | Structure | Amount | | |
|---|---|---|---|---|
| No. | Name | Yield | MS (ES+) m/z | ¹H NMR |
| 399 | 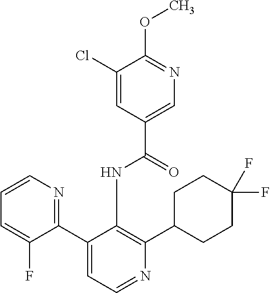 [SMILES: COc1ncc(C(=O)Nc2c(-c3ncccc3F)ccnc2C2CCC(F)(F)CC2)cc1Cl] 5-chloro-N-(2′-(4,4- difluorocyclohexyl)-3- fluoro-[2,4′-bipyridin]-3′- yl)-6-methoxynicotinamide | 0.062 g 39% | 477.2 (M + 1) | (400 MHz, DMSO-d₆) δ 9.90 (s, 1H), 8.63 (d, J = 4.9 Hz, 1H), 8.46 (d, J = 4.6 Hz, 1H), 8.33 (d, J = 2.3 Hz, 1H), 8.14 (d, J = 2.4 Hz, 1H), 7.84-7.79 (m, 1H), 7.49 (dt, J = 8.5, 4.3 Hz, 1H), 7.44 (dd, J = 4.8, 0.8 Hz, 1H), 3.55 (s, 3H), 3.17- 3.12 (m, 1H), 2.12-2.08 (m, 2H), 1.98-1.80 (m, 6H) |
| 400 | 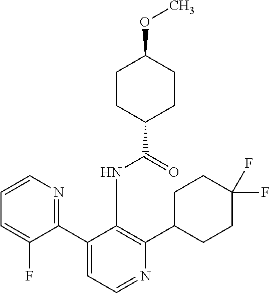 [SMILES: CO[C@H]1CC[C@H](C(=O)Nc2c(-c3ncccc3F)ccnc2C2CCC(F)(F)CC2)CC1] (anti)-N-(2′-(4,4- difluorocyclohexyl)-3- fluoro-[2,4′-bipyridin]-3′- yl)-4-methoxycyclohexane-1- carboxamide | 0.051 g 58% | 448.4 (M + 1) | (400 MHz, DMSO-d₆) δ 9.52 (s, 1H), 8.55 (d, J = 4.9 Hz, 1H), 8.49 (dd, J = 4.6, 1.5 Hz, 1H), 7.82 (td, J = 9.3, 1.2 Hz, 1H), 7.52 (dt, J = 8.5, 4.3 Hz, 1H), 7.35 (d, J = 4.8 Hz, 1H), 3.20 (s, 3H), 3.13-3.06 (m, 1H), 3.03-2.96 (m, 1H), 2.18-2.11 (m, 3H), 1.98-1.78 (m, 8H), 1.59-1.56 (m, 2H), 1.24-1.13 (m, 2H), 1.08-0.98 (m, 2H) |
| 401 | 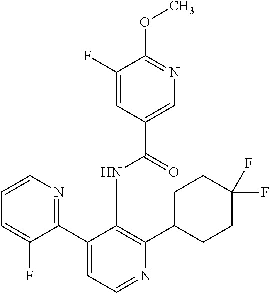 [SMILES: COc1ncc(C(=O)Nc2c(-c3ncccc3F)ccnc2C2CCC(F)(F)CC2)cc1F] N-(2′-(4,4- difluorocyclohexyl)-3- fluoro-[2,4′-bipyridin]-3′- yl)-5-fluoro-6- methoxynicotinamide | 0.044 g 29% | 461.2 (M + 1) | (400 MHz, DMSO-d₆) δ 10.18 (s, 1H), 8.65 (d, J = 4.9 Hz, 1H), 8.45 (dd, J = 3.2, 1.4 Hz, 1H), 8.37 (d, J = 1.9 Hz, 1H), 7.92 (dd, J = 11.1, 1.9 Hz, 1H), 7.81 (ddd, J = 10.0, 8.7, 1.2 Hz, 1H), 7.50-7.45 (m, 2H), 4.00 (s, 3H), 3.21-3.15 (m, 1H), 2.14-2.06 (m, 2H), 1.95-1.82 (m, 6H) |
| 402 | 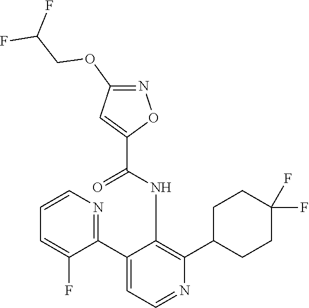 [SMILES: O=C(Nc1c(-c2ncccc2F)ccnc1C1CCC(F)(F)CC1)c1cc(OCC(F)F)no1] N-(2′-(4,4- difluorocyclohexyl)-3- fluoro-[2,4′-bipyridin]-3′- yl)-3-(2,2-difluoroethoxy)isoxazole- 5-carboxamide | 0.062 g 55% | 483.2 (M + 1) | (400 MHz, DMSO-d₆) δ 10.64 (s, 1H), 8.67 (d, J = 4.9 Hz, 1H), 8.47-8.45 (m, 1H), 7.83 (td, J = 9.4, 1.1 Hz, 1H), 7.51 (dt, J = 8.5, 4.3 Hz, 1H), 7.47 (dd, J = 4.9, 0.9 Hz, 1H), 6.96 (s, 1H), 6.42 (tt, J = 53.8, 3.1 Hz, 1H), 4.57 (td, J = 15.0, 3.1 Hz, 2H), 3.17-3.12 (m, 1H), 2.12-2.08 (m, 2H), 1.98-1.83 (m, 6H) |
| 403 | 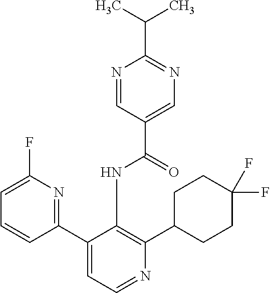 [SMILES: CC(C)c1ncc(C(=O)Nc2c(-c3cccc(F)n3)ccnc2C2CCC(F)(F)CC2)cn1] N-(2′-(4,4- difluorocyclohexyl)-6- fluoro-[2,4′-bipyridin]-3′- yl)-2-isopropylpyrimidine-5- carboxamide | 0.034 g 45% | 456.2 (M + 1) | (400 MHz, DMSO-d₆) δ 10.45 (s, 1H), 9.08 (s, 2H), 8.67 (d, J = 4.9 Hz, 1H), 8.08 (q, J = 8.0 Hz, 1H), 7.62 (dd, J = 7.5, 2.0 Hz, 1H), 7.53 (d, J = 4.9 Hz, 1H), 7.20 (dd, J = 8.2, 2.0 Hz, 1H), 3.22 (dquintet, J = 13.6, 6.8 Hz, 2H), 2.09-1.86 (m, 8H), 1.31 (d, J = 6.9 Hz, 6H) |
| 404 | 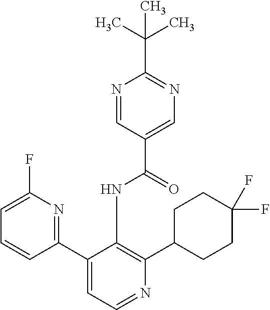 [SMILES: CC(C)(C)c1ncc(C(=O)Nc2c(-c3cccc(F)n3)ccnc2C2CCC(F)(F)CC2)cn1] 2-(tert-butyl)-N-(2′-(4,4- difluorocyclohexyl)-6- fluoro-[2,4′-bipyridin]-3′- yl)pyrimidine-5-carboxamide | 0.020 g 26% | 470.4 (M + 1) | (400 MHz, DMSO-d₆) δ 10.45 (s, 1H), 9.09 (s, 2H), 8.66 (d, J = 4.9 Hz, 1H), 8.08 (q, J = 8.0 Hz, 1H), 7.62 (dd, J = 7.4, 2.3 Hz, 1H), 7.53 (d, J = 4.9 Hz, 1H), 7.20 (dd, J = 8.2, 2.6 Hz, 1H), 3.26- 3.20 (m, 1H), 2.09-1.81 (m, 8H), 1.39 (s, 9H) |

### Example 405

### Synthesis of N-(2′-(4,4-difluoro-2-methylcyclohexyl)-3-fluoro-[2,4′-bipyridin]-3′-yl)-2-isopropylpyrimidine-5-carboxamide D1

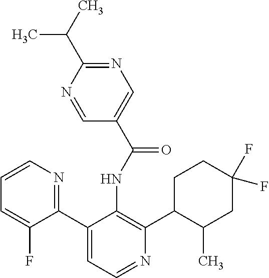 [SMILES: CC(C)c1ncc(C(=O)Nc2c(-c3ncccc3F)ccnc2C2CCC(F)(F)CC2C)cn1]

### Step 1. Preparation of tert-butyl (2′-(4,4-difluoro-2-methylcyclohexyl)-3-fluoro-[2,4′-bipyridin]-3′-yl)carbamate

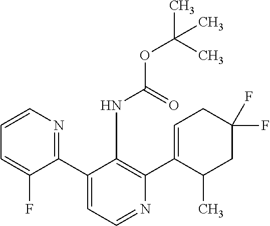 [SMILES: CC1CC(F)(F)CC=C1c1nccc(-c2ncccc2F)c1NC(=O)OC(C)(C)C]

A mixture of tert-butyl (2′-chloro-3-fluoro-[2,4′-bipyridin]-3′-yl)carbamate (0.45 g, 1.4 mmol) in 1,4-dioxane (9.3 mL) was degassed with nitrogen for 10 minutes. To the reaction mixture was added N-[(4,4-difluoro-2-methyl-cyclohexylidene)amino]-4-methyl-benzenesulfonamide (0.66 g, 2.1 mmol), tris(dibenzylideneacetone)dipalladium(0) (0.13 g, 0.14 mmol), tricyclohexylphosphine tetrafluoroborate (0.10 g, 0.28 mmol) and lithium tert-butoxide (0.33 g, 4.2 mmol). The reaction mixture was stirred at 110° C. for 16 h. After cooling to ambient temperature, the reaction mixture was diluted in water (30 mL) and the aqueous phase was extracted with ethyl acetate (3×20 mL). The combined organic phases were washed with brine (20 mL), dried over anhydrous magnesium sulfate, filtered, and concentrated in vacuo. The residue was purified by column chromatography, eluting with a gradient of 5 to 80% ethyl acetate in heptane, to afford the title compound as a yellow solid (0.35 g, 59% yield): MS (ES+) m/z 420.2 (M+1).

### Step 2. 2′-(4,4-difluoro-6-methylcyclohex-1-en-1-yl)-3-fluoro-[2,4′-bipyridin]-3′-amine

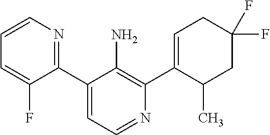 [SMILES: CC1CC(F)(F)CC=C1c1nccc(-c2ncccc2F)c1N]

To tert-butyl (2′-(4,4-difluoro-2-methylcyclohexyl)-3-fluoro-[2,4′-bipyridin]-3′-yl)carbamate (0.34 g, 0.81 mmol) was added 4.0 M hydrochloric acid in anhydrous dioxane (4.0 mL, 16 mmol) and 1,4-dioxane (3.2 mL), and the reaction mixture was stirred at ambient temperature for 16 h. The reaction mixture was diluted in saturated potassium carbonate (15 mL) and the aqueous phase was extracted with ethyl acetate (3×20 mL). The combined organic phase was washed with brine (20 mL), dried over anhydrous magnesium sulfate, filtered, and concentrated in vacuo. The resulting residue was used as is in the next step (0.18 g, 69% yield, crude): MS (ES+) m/z 320.4 (M+1).

### Step 3. Preparation of 2′-(4,4-difluoro-2-methylcyclohexyl)-3-fluoro-[2,4′-bipyridin]-3′-amine

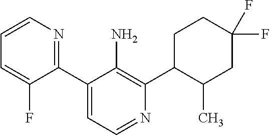 [SMILES: CC1CC(F)(F)CCC1c1nccc(-c2ncccc2F)c1N]

A mixture of 2′-(4,4-difluoro-6-methylcyclohex-1-en-1-yl)-3-fluoro-[2,4′-bipyridin]-3′-amine (0.18 g, 0.56 mmol) in ethanol (5.6 mL) and acetic acid (2.2 mL) was degassed with nitrogen for 10 minutes. To the reaction mixture was added ammonium formate (0.35 g, 5.6 mmol) and 10% palladium hydroxide (0.086 g, 0.61 mmol). The reaction mixture was stirred at 80° C. for 2.5 h. After cooling to ambient temperature, the mixture was diluted in ethyl acetate (25 mL) and filtered through a bed of Celite. The filtrate was washed with sodium bicarbonate (15 mL) and brine (15 mL), dried with anhydrous magnesium sulfate, and concentrated in vacuo. The resulting residue was used as is in the next step (0.060 g, 33% yield): MS (ES+) m/z 322.4 (M+1).

### Step 4. Preparation of N-(2′-(4,4-difluoro-2-methylcyclohexyl)-3-fluoro-[2,4′-bipyridin]-3′-yl)-2-isopropylpyrimidine-5-carboxamide

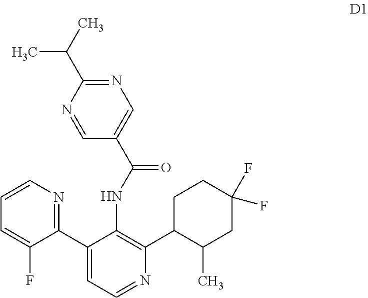 [SMILES: CC(C)c1ncc(C(=O)Nc2c(-c3ncccc3F)ccnc2C2CCC(F)(F)CC2C)cn1]

To a mixture of 2′-(4,4-difluoro-2-methylcyclohexyl)-3-fluoro-[2,4′-bipyridin]-3′-amine (0.058 g, 0.18 mmol) in anhydrous tetrahydrofuran (1.8 mL) was added N,N-diisopropylethylamine (0.19 mL, 1.1 mmol), 2-chloro-1-methylpyridinium iodide (0.14 g, 0.54 mmol) and 2-isopropylpyrimidine-5-carboxylic acid (0.045 g, 0.27 mmol). The reaction mixture was stirred at 65° C. for 8 h. After cooling to ambient temperature, to the reaction mixture was added in methanol (3 mL) and 10 M sodium hydroxide (1 mL) and the mixture was stirred at ambient temperature for 10 minutes. The reaction mixture was diluted with ethyl acetate (10 mL), and the organic phase was washed with 1 M sodium hydroxide (20 mL) and saturated ammonium chloride (20 mL), dried with anhydrous magnesium sulfate, filtered, and concentrated in vacuo. Purification of the residue by preparative reverse phase HPLC, using a gradient of 10-85% acetonitrile in water containing 0.5% of formic acid as eluent, afforded the title compound as a colorless solid (0.0095 g, 11% yield): ¹H NMR (400 MHz, DMSO-d₆) δ 10.44 (d, J=0.2 Hz, 1H), 8.93 (s, 2H), 8.67 (d, J=4.9 Hz, 1H), 8.47 (d, J=4.3 Hz, 1H), 7.84 (t, J=9.3 Hz, 1H), 7.49 (q, J=5.4 Hz, 2H), 3.50 (s, 1H), 3.19 (dquintet, J=13.8, 6.9 Hz, 1H), 2.47-2.42 (m, 1H), 2.35-2.22 (m, 2H), 2.14-2.08 (m, 1H), 2.01-1.82 (m, 3H), 1.28 (d, J=6.9 Hz, 6H), 0.75 (d, J=6.9 Hz, 3H); MS (ES+) m/z 470.3 (M+1).

In a similar manner as described in EXAMPLE 29, utilizing the appropriately substituted starting materials and intermediates, the following compounds were prepared:

| Example | Structure | Amount | | |
|---|---|---|---|---|
| No. | Name | Yield | MS (ES+) m/z | ¹H NMR |
| 406 | 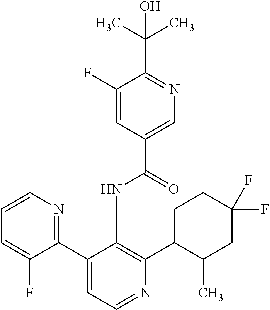 [SMILES: CC1CC(F)(F)CCC1c1nccc(-c2ncccc2F)c1NC(=O)c1cnc(C(C)(C)O)c(F)c1] N-(2′-(4,4-diluoro-2- methylcyclohexyl)-3- fluoro-[2,4′-bipyridin]-3′- yl)-5-fluoro-6-(2- hydroxypropan-2- yl)nicotinamide | 0.011 g 2.8% | 503.3 (M + 1) | (400 MHz, DMSO-d₆) δ 10.38 (s, 1H), 8.68 (d, J = 4.9 Hz, 1H), 8.63 (s, 1H), 8.48-8.47 (m, 1H), 7.90-7.80 (m, 2H), 7.52-7.45 (m, 2H), 5.34 (s, 1H), 2.86-2.80 (m, 1H), 2.30-2.21 (m, 1H), 2.12-2.05 (m, 2H), 1.97-1.79 (m, 3H), 1.77-1.60 (m, 1H), 1.52 (s, 6H), 0.64 (d, J = 6.4 Hz, 3H) |
| 407 | 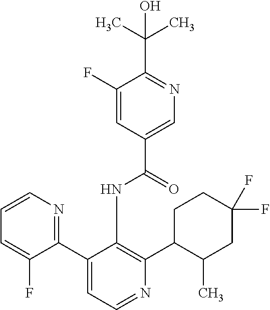 [SMILES: CC1CC(F)(F)CCC1c1nccc(-c2ncccc2F)c1NC(=O)c1cnc(C(C)(C)O)c(F)c1] N-(2′-(4,4-difluoro-2- methylcyclohexyl)-3- fluoro-[2,4′-bipyridin]-3′- yl)-5-fluoro-6-(2- hydroxypropan-2- yl)nicotinamide | 0.019 g 4.9% | 503.2 (M + 1) | (400 MHz, DMSO-d₆) δ 10.39 (s, 1H), 8.67 (d, J = 4.9 Hz, 1H), 8.61 (t, J = 1.6 Hz, 1H), 8.47- 8.46 (m, 1H), 7.89-7.80 (m, 2H), 7.51-7.47 (m, 2H), 5.34 (s, 1H), 3.49- 3.47 (m, 1H), 2.48-2.42 (m, 1H), 2.36-2.22 (m, 2H), 2.14-2.06 (m, 1H), 1.99-1.80 (m, 3H), 1.51 (s, 6H), 0.75 (d, J = 6.9 Hz, 3H) |

### Example 408

### Synthesis of 2-(tert-butyl)-N-(2′-(4,4-difluorocyclohexyl)-6-methoxy-[2,4′-bipyridin]-3′-yl)pyrimidine-5-carboxamide

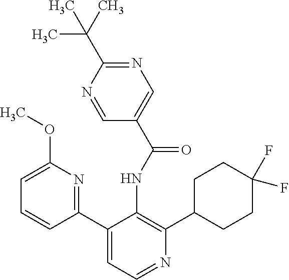 [SMILES: COc1cccc(-c2ccnc(C3CCC(F)(F)CC3)c2NC(=O)c2cnc(C(C)(C)C)nc2)n1]

### Preparation of 2-(tert-butyl)-N-(2′-(4,4-difluorocyclohexyl)-6-methoxy-[2,4′-bipyridin]-3′-yl)pyrimidine-5-carboxamide

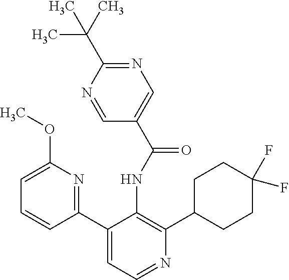 [SMILES: COc1cccc(-c2ccnc(C3CCC(F)(F)CC3)c2NC(=O)c2cnc(C(C)(C)C)nc2)n1]

To a mixture of 2-(tert-butyl)-N-(2′-(4,4-difluorocyclohexyl)-6-fluoro-[2,4′-bipyridin]-3′-yl)pyrimidine-5-carboxamide (0.015 g, 0.032 mmol) in anhydrous DMSO (0.29 mL) was added methanol (0.29 mL, 0.032 mmol) and 60% sodium hydride dispersion in mineral oil (0.013 g, 0.32 mmol). The solution was stirred at 70° C. for 12 h. The reaction mixture was diluted with ethyl acetate (10 mL) and washed with saturated ammonium chloride solution (2×20 mL). The organic layer was dried over anhydrous magnesium sulfate, filtered, and concentrated in vacuo. Purification of the residue by column chromatography, eluting with 10-90% ethyl acetate in heptane, provided the title compound as a colorless solid (0.011 g, 72% yield): ¹H-NMR (400 MHz; DMSO-d₆) δ 10.38 (s, 1H), 9.09 (s, 2H), 8.64 (d, J=4.9 Hz, 1H), 7.77 (dd, J=8.3, 7.4 Hz, 1H), 7.55 (d, J=4.9 Hz, 1H), 7.26-7.24 (m, 1H), 6.82 (dd, J=8.3, 0.5 Hz, 1H), 3.79 (s, 3H), 3.25-3.17 (m, 1H), 2.12-1.98 (m, 3H), 1.97-1.86 (m, 5H), 1.39 (s, 9H); MS (ES+) m/z 482.4 (M+1).

### Example 409 and 410

### Synthesis of 6-(3,3-difluorocyclobutoxy)-N-(2′-(5,5-difluorotetrahydro-2H-pyran-2-yl)-3-fluoro-[2,4′-bipyridin]-3′-yl)-5-fluoronicotinamide P1/P2/

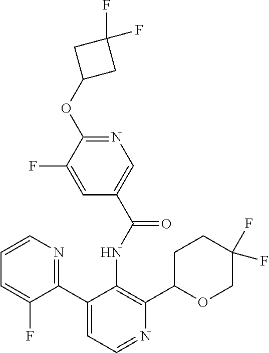 [SMILES: O=C(Nc1c(-c2ncccc2F)ccnc1C1CCC(F)(F)CO1)c1cnc(OC2CC(F)(F)C2)c(F)c1]

### Step 1. Preparation of tert-butyl (2′-(5,5-difluorotetrahydro-2H-pyran-2-yl)-3-fluoro-[2,4′-bipyridin]-3′-yl)carbamate

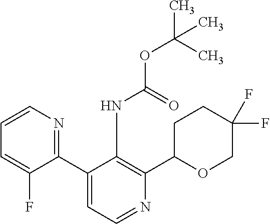 [SMILES: CC(C)(C)OC(=O)Nc1c(-c2ncccc2F)ccnc1C1CCC(F)(F)CO1]

To a mixture of (4,4″-di-t-butyl-2,2″-bipyridine)bis[3,5-difluoro-2-[5-trifluoromethyl-2-pyridinyl-kN)phenyl-kC]iridium(III) hexafluorophosphate (0.012 g, 0.011 mmol), dichloro(dimethoxyethane)nickel (0.024 g, 0.11 mmol), 4,4′-di-tert-butyl-2,2′-bipyridine (0.044 g, 0.16 mmol), tert-butyl N-[2-chloro-4-(3-fluoro-2-pyridyl)-3-pyridyl]carbamate (0.35 g, 1.1 mmol), cesium carbonate (0.63 g, 1.9 mmol) and 5,5-difluorooxane-2-carboxylic acid (0.27 g, 1.6 mmol) was added N,N-dimethylformamide (22 mL). The mixture was purged with nitrogen for 10 s and sealed. The reaction mixture was then stirred in front of Kessil PR160L lights (440 nm) for 8 h. The reaction mixture was diluted with ethyl acetate (300 mL). The organic layer was washed with saturated ammonium chloride (2×100 mL), dried over anhydrous magnesium sulfate, filtered, and concentrated in vacuo. The residue was purified by column chromatography, eluting with a gradient of 0 to 40% of ethyl acetate in heptane, to provide the title compound as a yellow solid (0.36 g, 82% yield): MS (ES+) m/z 410.4 (M+1).

### Step 2. Preparation of 2′-(5,5-difluorotetrahydro-2H-pyran-2-yl)-3-fluoro-[2,4′-bipyridin]-3′-amine

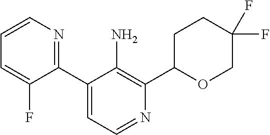 [SMILES: Nc1c(-c2ncccc2F)ccnc1C1CCC(F)(F)CO1]

A flask containing tert-butyl (2′-(5,5-difluorotetrahydro-2H-pyran-2-yl)-3-fluoro-[2,4′-bipyridin]-3′-yl)carbamate (0.36 g, 0.88 mmol) was charged with 4M hydrogen chloride in 1,4-dioxane (4.4 mL) and 1,4-dioxane (3.5 mL). The reaction vessel was sealed and heated to 45° C. for 5 h. The heterogeneous reaction mixture was diluted with ethyl acetate (30 mL) and 5M sodium hydroxide (60 mL). The mixture was sonicated for 30 min. The organic layer was separated and washed with brine (50 mL), dried over anhydrous magnesium sulfate, filtered, and concentrated in vacuo. The residue was purified by column chromatography, eluting with a gradient of 5 to 60% of ethyl acetate in heptane, to provide the title compound as a yellow solid (0.22 g, 81% yield): MS (ES+) m/z 310.2 (M+1).

### Step 3. Preparation of N-(2′-(5,5-difluorotetrahydro-2H-pyran-2-yl)-3-fluoro-[2,4′-bipyridin]-3′-yl)-5,6-difluoronicotinamide

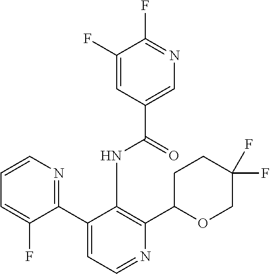 [SMILES: O=C(Nc1c(-c2ncccc2F)ccnc1C1CCC(F)(F)CO1)c1cnc(F)c(F)c1]

A vial containing 2′-(5,5-difluorotetrahydro-2H-pyran-2-yl)-3-fluoro-[2,4′-bipyridin]-3′-amine (0.18 g, 0.59 mmol), 2-chloro-1-methylpyridinium iodide (0.45 g, 1.8 mmol), 2,3-difluoropyridine-5-carboxylic acid (0.14 g, 0.88 mmol), and anhydrous THF (5.9 mL) was charged with N,N-diisopropylethylamine (0.61 mL, 3.5 mmol). The reaction vessel was sealed and stirred at 68° C. for 20 h. The reaction mixture was diluted with ethyl acetate (20 mL). The organic layer was washed with 1M sodium hydroxide (20 mL), saturated ammonium chloride (2×30 mL) and water (30 mL), then dried over anhydrous magnesium sulfate, filtered, and concentrated in vacuo. The residue was purified by column chromatography, eluting with a gradient of 5 to 95% of ethyl acetate in heptane, to provide the title compound as a light-yellow solid (0.13 g, 47% yield): MS (ES+) m/z 451.4 (M+1).

### Step 4. Preparation of rac-6-(3,3-difluorocyclobutoxy)-N-(2′-(5,5-difluorotetrahydro-2H-pyran-2-yl)-3-fluoro-[2,4′-bipyridin]-3′-yl)-5-fluoronicotinamide

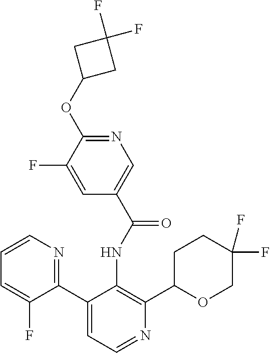 [SMILES: O=C(Nc1c(-c2ncccc2F)ccnc1C1CCC(F)(F)CO1)c1cnc(OC2CC(F)(F)C2)c(F)c1]

To a mixture of N-(2′-(5,5-difluorotetrahydro-2H-pyran-2-yl)-3-fluoro-[2,4′-bipyridin]-3′-yl)-5,6-difluoronicotinamide (0.12 g, 0.27 mmol) in anhydrous dimethylformamide (1.1 mL) was added 3,3-difluorocyclobutan-1-ol (1.2 mL, 0.29 mmol) and 60% sodium hydride dispersion in mineral oil (0.11 g, 2.7 mmol) at 0° C. The reaction mixture was stirred at ambient temperature for 4 h and then diluted with ethyl acetate (10 mL) and washed with saturated ammonium chloride solution (2×20 mL). The organic layer was dried over anhydrous magnesium sulfate, filtered, and concentrated in vacuo. Purification of the residue by column chromatography, eluting with a gradient of 5 to 70% ethyl acetate in heptane, provided the title compound as a pale pink solid (0.70 g, 46% yield): ¹H-NMR (400 MHz; DMSO-d₆) δ 10.25 (s, 1H), 8.70 (d, J=4.9 Hz, 1H), 8.45 (d, J=4.5 Hz, 1H), 8.37 (d, J=1.6 Hz, 1H), 7.96 (dd, J=10.9, 1.7 Hz, 1H), 7.84-7.80 (m, 1H), 7.60 (d, J=4.7 Hz, 1H), 7.50 (dt, J=8.5, 4.3 Hz, 1H), 5.29-5.20 (m, 1H), 4.91 (d, J=10.5 Hz, 1H), 3.95-3.88 (m, 1H), 3.79-3.68 (m, 1H), 3.25-3.14 (m, 2H), 2.90-2.78 (m, 2H), 2.38-2.13 (m, 3H), 2.02-1.99 (m, 1H); MS (ES+) m/z 539.2 (M+1).

### Step 4. Preparation of 6-(3,3-difluorocyclobutoxy)-N-(2′-(5,5-difluorotetrahydro-2H-pyran-2-yl)-3-fluoro-[2,4′-bipyridin]-3′-yl)-5-fluoronicotinamide P1/P2

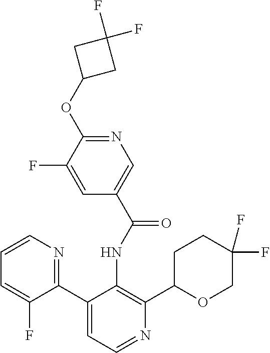 [SMILES: O=C(Nc1c(-c2ncccc2F)ccnc1C1CCC(F)(F)CO1)c1cnc(OC2CC(F)(F)C2)c(F)c1]

Rac-6-(3,3-difluorocyclobutoxy)-N-(2′-(5,5-difluorotetrahydro-2H-pyran-2-yl)-3-fluoro-[2,4′-bipyridin]-3′-yl)-5-fluoronicotinamide (0.053 g, 0.098 mmol) was purified by chiral SFC (column: DAICEL CHIRALPAK OD-H (250 mm×30 mm, 5 μm), eluting with 20% of methanol containing 0.1% ammonium hydroxide in supercritical carbon dioxide, to afford peak 1 (retention time=1.138 min) as a colorless solid (0.015 g, 27% yield, 99% ee): ¹H NMR (400 MHz, CDCl₃) δ 9.71 (s, 1H), 8.59 (d, J=4.8 Hz, 1H), 8.44 (d, J=4.4 Hz, 1H), 8.36 (d, J=1.6 Hz, 1H), 7.73 (dd, J=1.6, 10.0 Hz, 1H), 7.61-7.49 (m, 2H), 7.32 (td, J=4.0, 8.4 Hz, 1H), 5.33-5.20 (m, 1H), 4.87 (dd, J=2.8, 10.0 Hz, 1H), 4.15-4.07 (m, 1H), 3.80-3.65 (m, 1H), 3.26-3.11 (m, 2H), 2.95-2.73 (m, 2H), 2.43-2.18 (m, 3H), 2.16-1.98 (m, 1H); MS (ES+) m/z 539.2 (M+1).

Peak 2 (retention time=1.330 min) afforded a colorless solid (0.015 g, 27% yield, 99% ee): ¹H NMR (400 MHz, CDCl₃) δ 9.71 (s, 1H), 8.59 (d, J=4.8 Hz, 1H), 8.44 (d, J=4.4 Hz, 1H), 8.36 (d, J=0.8 Hz, 1H), 7.76-7.69 (m, 1H), 7.60-7.50 (m, 2H), 7.32 (td, J=4.0, 8.4 Hz, 1H), 5.36-5.19 (m, 1H), 4.87 (dd, J=2.4, 10.0 Hz, 1H), 4.17-4.06 (m, 1H), 3.80-3.66 (m, 1H), 3.25-3.11 (m, 2H), 2.92-2.75 (m, 2H), 2.42-2.19 (m, 3H), 2.16-1.98 (m, 1H); MS (ES+) m/z 539.2 (M+1).

### Example 411

### Synthesis of N-(2′-(4,4-difluorocyclohexyl)-3-fluoro-[2,4′-bipyridin]-3′-yl)-2-isopropoxypyrimidine-5-carboxamide

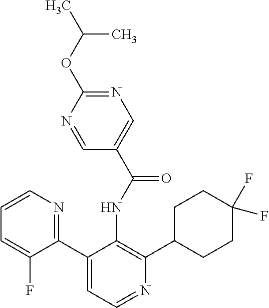 [SMILES: CC(C)Oc1ncc(C(=O)Nc2c(-c3ncccc3F)ccnc2C2CCC(F)(F)CC2)cn1]

### Step 1. Preparation 2-chloro-N-(2′-(4,4-difluorocyclohexyl)-3-fluoro-[2,4′-bipyridin]-3′-yl)pyrimidine-5-carboxamide

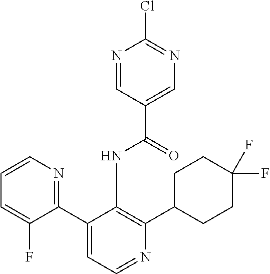 [SMILES: O=C(Nc1c(-c2ncccc2F)ccnc1C1CCC(F)(F)CC1)c1cnc(Cl)nc1]

A mixture containing 2′-(4,4-difluorocyclohexyl)-3-fluoro-[2,4′-bipyridin]-3′-amine (0.15 g, 0.49 mmol), 2-chloro-1-methylpyridinium iodide (0.37 g, 1.5 mmol), 2-chloropyrimidine-5-carboxylic acid (0.12 g, 0.73 mmol), and anhydrous tetrahydrofuran (4.9 mL) was charged with N,N-diisopropylethylamine (0.51 mL, 2.9 mmol). The reaction vessel was sealed and stirred at 68° C. for 20 h. The reaction mixture was diluted with ethyl acetate (20 mL). The organic layer was washed with 1M sodium hydroxide (20 mL), saturated ammonium chloride (2×30 mL), and water (30 mL), then dried over anhydrous magnesium sulfate, filtered, and concentrated in vacuo. The resulting solid was used in the next step without further purification (0.097 g, 44% yield): MS (ES+) m/z 449.4 (M+1), 451.4 (M+1).

### Step 2. Preparation of N-(2′-(4,4-difluorocyclohexyl)-3-fluoro-[2,4′-bipyridin]-3′-yl)-2-isopropoxypyrimidine-5-carboxamide

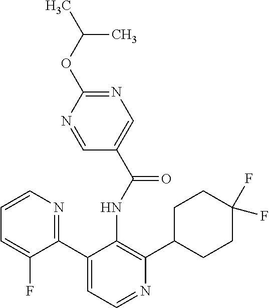 [SMILES: CC(C)Oc1ncc(C(=O)Nc2c(-c3ncccc3F)ccnc2C2CCC(F)(F)CC2)cn1]

To a mixture of 2-chloro-N-(2′-(4,4-difluorocyclohexyl)-3-fluoro-[2,4′-bipyridin]-3′-yl)pyrimidine-5-carboxamide (0.097 g, 0.22 mmol) in anhydrous DMF (1.1 mL) was added 2-propanol (2.8 mL, 2.2 mmol) and 60% sodium hydride dispersion in mineral oil (0.043 g, 1.8 mmol). The solution was stirred at 40° C. for 2 h. The reaction mixture was diluted with ethyl acetate (10 mL) and washed with saturated ammonium chloride solution (2×20 mL). The organic layer was dried over anhydrous magnesium sulfate, filtered, and concentrated in vacuo. Purification of the residue by column chromatography, eluting with a gradient of 5 to 80% ethyl acetate in heptane, provided the title compound as an off-white solid (0.035 g, 32% yield): ¹H-NMR (400 MHz; DMSO-d₆) δ 10.24 (s, 1H), 8.84 (s, 2H), 8.65 (d, J=4.9 Hz, 1H), 8.46 (dt, J=3.0, 1.5 Hz, 1H), 7.82 (ddd, J=10.1, 8.7, 1.2 Hz, 1H), 7.51-7.46 (m, 2H), 5.26 (7, J=6.2 Hz, 1H), 3.22-3.17 (m, 1H), 2.14-2.05 (m, 2H), 1.97-1.84 (m, 6H), 1.34 (d, J=6.2 Hz, 6H); MS (ES+) m/z 472.2 (M+1).

In a similar manner as described in examples disclosed herein, utilizing the appropriately substituted starting materials and intermediates, the following compounds were prepared:

| Example | Structure | Amount | | |
|---|---|---|---|---|
| No. | Name | Yield | MS (ES+) m/z | ¹H NMR |
| 412 | 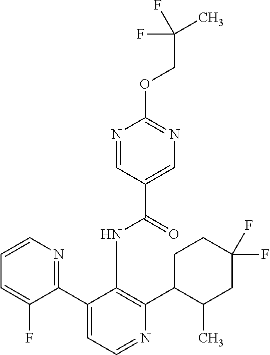 N-(2′-(4,4- difluorocyclohexyl)-3- fluoro-[2,4′-bipyridin]-3′- yl)-2-(2,2-difluoropropoxy) pyrimidine-5-carboxamide | 0.049 g 76% | 508.2 (M + 1) | (400 MHz, MeOD) δ 8.93 (s, 2H), 8.67 (d, J = 5.0 Hz, 1H), 8.47 (dt, J = 4.7, 1.5 Hz, 1H), 7.72 (ddd, J = 10.0, 8.5, 1.4 Hz, 1H), 7.51 (dd, J = 5.0, 1.4 Hz, 1H), 7.50-7.46 (m, 1H), 4.68 (t, J = 12.1 Hz, 2H), 3.26-3.20 (m, 1H), 2.20-2.04 (m, 4H), 1.95 (d, J = 13.3 Hz, 3H), 1.90-1.84 (m, 1H), 1.78 (t, J = 18.8 Hz, 3H) |
| 413 | 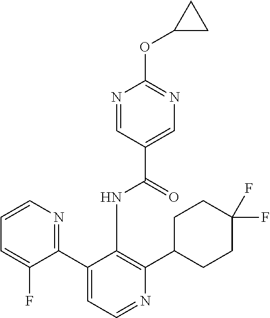 [SMILES: O=C(Nc1c(-c2ncccc2F)ccnc1C1CCC(F)(F)CC1)c1cnc(OC2CC2)nc1] 2-cyclopropoxy-N-(2′- (4,4-difluorocyclohexyl)- 3-fluoro-[2,4′-bipyridin]- 3′-yl)pyrimidine-5-carboxamide | 0.0090 g 16% | 470.2 (M + 1) | (400 MHz, MeOD) δ 8.91 (s, 2H), 8.66 (s, 1H), 8.48 (d, J = 4.6 Hz, 1H), 7.75-7.70 (m, 1H), 7.52-7.46 (m, 2H), 4.46 (tt, J = 6.1, 3.1 Hz, 1H), 3.26-3.19 (m, 1H), 2.21- 2.03 (m, 4H), 1.97-1.81 (m, 4H), 0.90-0.85 (m, 2H), 0.84-0.80 (m, 2H) |
| 414 | 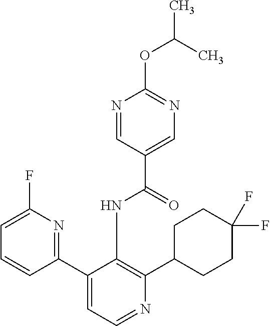 [SMILES: CC(C)Oc1ncc(C(=O)Nc2c(-c3cccc(F)n3)ccnc2C2CCC(F)(F)CC2)cn1] N-(2‘-(4,4- difluorocyclohexyl)-6- fluoro-[2,4′-bipyridin]-3′-yl)-2- isopropoxypyrimidine-5- carboxamide | 0.010 g 35% | 472.2 (M + 1) | ¹H-NMR (400 MHz; DMSO-d) δ 10.29 (s, 1H), 8.96 (s, 2H), 8.65 (d, J = 4.9 Hz, 1H), 8.06 (q, J = 8.0 Hz, 1H), 7.60 (dd, J = 7.4, 2.5 Hz, 1H), 7.53 (d, J = 4.9 Hz, 1H), 7.20 (dd, J = 8.2, 2.6 Hz, 1H), 5.29 (7, J = 6.2 Hz, 1H), 3.25-3.17 (m, 1H), 2.12-2.04 (m, 2H), 2.01-1.81 (m, 6H), 1.35 (d, J = 6.2 Hz, 6H) |

### Example 415

### Synthesis of N-(2′-(4,4-difluorocyclohexyl)-3-fluoro-[2,4′-bipyridin]-3′-yl)-6-isopropoxynicotinamide

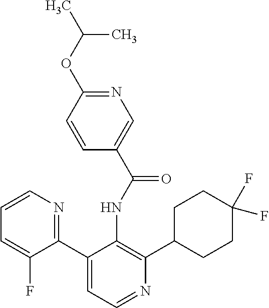 [SMILES: CC(C)Oc1ccc(C(=O)Nc2c(-c3ncccc3F)ccnc2C2CCC(F)(F)CC2)cn1]

### Step 1. Preparation N-(2′-(4,4-difluorocyclohexyl)-3-fluoro-[2,4′-bipyridin]-3′-yl)-6-fluoronicotinamide

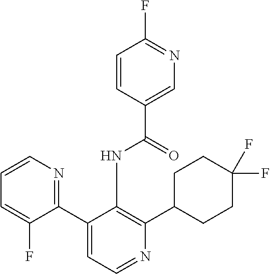 [SMILES: O=C(Nc1c(-c2ncccc2F)ccnc1C1CCC(F)(F)CC1)c1ccc(F)nc1]

A mixture containing 2-(4,4-difluorocyclohexyl)-4-(3-fluoro-2-pyridyl)pyridin-3-amine (0.10 g, 0.33 mmol), 2-chloro-1-methylpyridinium iodide (0.25 g, 0.98 mmol), 6-fluoronicotinic acid (0.069 g, 0.49 mmol), and anhydrous tetrahydrofuran (3.3 mL) was charged with N,N-diisopropylethylamine (0.34 mL, 2.0 mmol). The reaction vessel was sealed and stirred at 68° C. for 20 h. The reaction mixture was diluted with ethyl acetate (20 mL). The organic layer was washed with 1M sodium hydroxide (20 mL), saturated ammonium chloride (2×30 mL), and water (30 mL), then dried over anhydrous magnesium sulfate, filtered, and concentrated in vacuo. The resulting solid was used in the next step without further purification (0.14 g, quant. yield, crude): MS (ES+) m/z 431.4 (M+1).

### Step 2. Preparation of N-(2′-(4,4-difluorocyclohexyl)-3-fluoro-[2,4′-bipyridin]-3′-yl)-6-isopropoxynicotinamide

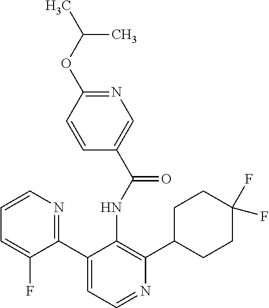 [SMILES: CC(C)Oc1ccc(C(=O)Nc2c(-c3ncccc3F)ccnc2C2CCC(F)(F)CC2)cn1]

To a mixture of N-(2′-(4,4-difluorocyclohexyl)-3-fluoro-[2,4′-bipyridin]-3′-yl)-6-fluoronicotinamide (0.14 g, 0.33 mmol) in anhydrous dimethylformamide (3.3 mL) was added 2-propanol (3.3 mL, 0.33 mmol) and 60% sodium hydride dispersion in mineral oil (0.13 g, 3.3 mmol). The solution was stirred at 40° C. for 3 h. The reaction mixture was diluted with ethyl acetate (10 mL) and washed with saturated ammonium chloride solution (2×20 mL). The organic layer was dried over anhydrous magnesium sulfate, filtered, and concentrated in vacuo. Purification of the residue by column chromatography, eluting with a gradient of 5 to 80% ethyl acetate in heptane, provided the title compound as an off-white solid (0.088 g, 55% yield): ¹H-NMR (400 MHz; DMSO-d₆) δ 10.09 (s, 1H), 8.63 (d, J=4.9 Hz, 1H), 8.53 (d, J=2.4 Hz, 1H), 8.44 (d, J=4.6 Hz, 1H), 7.95 (dd, J=8.7, 2.5 Hz, 1H), 7.82-7.77 (m, 1H), 7.46 (dt, J=12.6, 4.3 Hz, 2H), 6.79 (d, J=8.7 Hz, 1H), 5.30 (7, J=6.2 Hz, 1H), 3.22-3.16 (m, 1H), 2.16-2.08 (m, 2H), 1.97-1.80 (m, 6H), 1.30 (d, J=6.2 Hz, 6H); MS (ES+) m/z 471.2 (M+1).

### Example 416

### Synthesis of N-(2′-(4,4-difluorocyclohexyl)-3-fluoro-[2,4′-bipyridin]-3′-yl)-5-fluoro-6-isopropoxynicotinamide

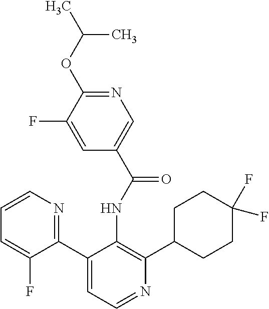 [SMILES: CC(C)Oc1ncc(C(=O)Nc2c(-c3ncccc3F)ccnc2C2CCC(F)(F)CC2)cc1F]

### Step 1. Preparation N-(2′-(4,4-difluorocyclohexyl)-3-fluoro-[2,4′-bipyridin]-3′-yl)-5,6-difluoronicotinamide

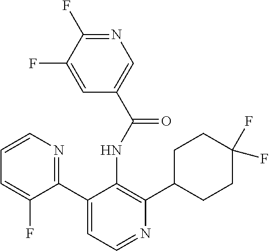 [SMILES: O=C(Nc1c(-c2ncccc2F)ccnc1C1CCC(F)(F)CC1)c1cnc(F)c(F)c1]

A mixture of 2′-(4,4-difluorocyclohexyl)-3-fluoro-[2,4′-bipyridin]-3′-amine (0.30 g, 0.98 mmol), 2-chloro-1-methylpyridinium iodide (0.75 g, 2.9 mmol), 2,3-difluoropyridine-5-carboxylic acid (0.23 g, 1.5 mmol), and anhydrous tetrahydrofuran (8.1 mL) was charged with N,N-diisopropylethylamine (1.0 mL, 5.9 mmol). The reaction vessel was sealed and stirred at 68° C. for 20 h. After cooling to ambient temperature, the mixture was diluted with methanol (5 mL) and 1M sodium hydroxide (2 mL). The mixture was stirred at ambient temperature for 30 min and then diluted with ethyl acetate (20 mL). The organic layer was washed with water (20 mL), saturated ammonium chloride (2×30 mL), and brine (30 mL), then dried over anhydrous magnesium sulfate, filtered, and concentrated in vacuo. Purification of the residue by column chromatography, eluting with a gradient of 5 to 95% ethyl acetate in heptane, provided the title compound as a yellow solid (0.035 g, 32% yield) (0.24 g, 55% yield): MS (ES+) m/z 449.4 (M+1).

### Step 2. Preparation of N-(2′-(4,4-difluorocyclohexyl)-3-fluoro-[2,4′-bipyridin]-3′-yl)-5-fluoro-6-isopropoxynicotinamide

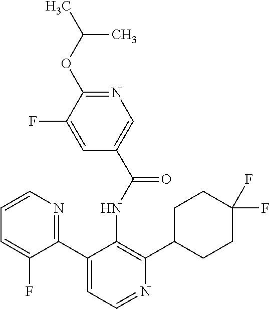 [SMILES: CC(C)Oc1ncc(C(=O)Nc2c(-c3ncccc3F)ccnc2C2CCC(F)(F)CC2)cc1F]

To a mixture of 2-chloro-N-(2′-(4,4-difluorocyclohexyl)-3-fluoro-[2,4′-bipyridin]-3′-yl)pyrimidine-5-carboxamide (0.24 g, 0.54 mmol) in anhydrous DMF (5.4 mL) was added 2-propanol (5.4 mL, 0.54 mmol) and 60% sodium hydride dispersion in mineral oil (0.22 g, 5.4 mmol). The solution was stirred at 40° C. for 2 h. The reaction mixture was diluted with ethyl acetate (10 mL) and washed with saturated ammonium chloride solution (2×20 mL). The organic layer was dried over anhydrous magnesium sulfate, filtered, and concentrated in vacuo. Purification of the residue by column chromatography, eluting with 5 to 80% ethyl acetate in heptane, provided the title compound as a colorless solid (0.13 g, 48% yield): ¹H-NMR (400 MHz; DMSO-d₆) S 10.15 (s, 1H), 8.65 (d, J=4.9 Hz, 1H), 8.45 (d, J=4.5 Hz, 1H), 8.36 (d, J=1.7 Hz, 1H), 7.89 (dd, J=11.1, 1.7 Hz, 1H), 7.81 (t, J=9.3 Hz, 1H), 7.48 (dt, J=12.7, 4.3 Hz, 2H), 5.38 (7, J=6.2 Hz, 1H), 3.20-3.18 (m, 1H), 2.12-2.08 (m, 2H), 1.98-1.82 (m, 6H), 1.35 (d, J=6.2 Hz, 6H); MS (ES+) m/z 489.2 (M+1).

In a similar manner as described in examples disclosed herein, utilizing the appropriately substituted starting materials and intermediates, the following compounds were prepared:

| Example | Structure | Amount | | |
|---|---|---|---|---|
| No. | Name | Yield | MS (ES+) m/z | ¹H NMR |
| 417 | 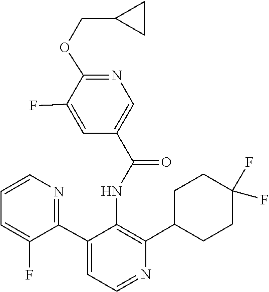 [SMILES: O=C(Nc1c(-c2ncccc2F)ccnc1C1CCC(F)(F)CC1)c1cnc(OCC2CC2)c(F)c1] 6-(cyclopropylmethoxy)- N-(2′-(4,4-difluorocyclohexyl)-3- fluoro-[2,4′-bipyridin]-3′- yl)-5-fluoronicotinamide | 0.018 g 19% | 501.2 (M + 1) | (400 MHz, DMSO-d₆) δ 10.16 (s, 1H), 8.65 (d, J = 4.9 Hz, 1H), 8.45-8.44 (m, 1H), 8.34 (d, J = 1.9 Hz, 1H), 7.91 (dd, J = 11.1, 1.9 Hz, 1H), 7.80 (td, J = 9.4, 1.1 Hz, 1H), 7.50- 7.45 (m, 2H), 4.25 (d, J = 7.3 Hz, 2H), 3.20-3.15 (m, 1H), 2.13-2.08 (m, 2H), 1.98-1.81 (m, 6H), 1.34-1.24 (m, 1H), 0.61-0.56 (m, 2H), 0.39-0.35 (m, 2H) |
| 418 | 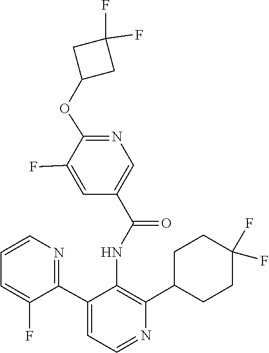 [SMILES: O=C(Nc1c(-c2ncccc2F)ccnc1C1CCC(F)(F)CC1)c1cnc(OC2CC(F)(F)C2)c(F)c1] 6-(3,3- difluorocyclobutoxy)-N- (2′-(4,4-difluorocyclohexyl)-3- fluoro-[2,4′-bipyridin]-3′- yl)-5-fluoronicotinamide | 0.077 g 90% | 537.2 (M + 1) | (400 MHz, DMSO-d₆) δ 10.21 (s, 1H), 8.65 (d, J = 4.9 Hz, 1H), 8.45-8.44 (m, 1H), 7.95 (dd, J = 10.9, 1.9 Hz, 1H), 7.83-7.78 (m, 1H), 7.51-7.45 (m, 2H), 5.29-5.20 (m, 1H), 3.27-3.15 (m, 3H), 2.91-2.76 (m, 2H), 2.12-2.08 (m, 2H), 1.98-1.82 (m, 6H) |

### Example 419

### Synthesis of 6-(2-azabicyclo[2.1.1]hexan-2-yl)-N-(2′-(4,4-difluorocyclohexyl)-3-fluoro-[2,4′-bipyridin]-3′-yl)-5-fluoronicotinamide

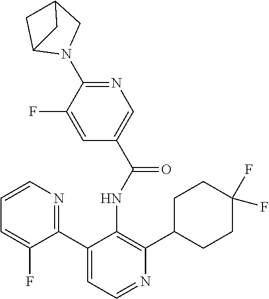 [SMILES: O=C(Nc1c(-c2ncccc2F)ccnc1C1CCC(F)(F)CC1)c1cnc(N2CC3CC2C3)c(F)c1]

### Step 1. Preparation of 6-(2-azabicyclo[2.1.1]hexan-2-yl)-N-(2′-(4,4-difluorocyclohexyl)-3-fluoro-[2,4′-bipyridin]-3′-yl)-5-fluoronicotinamide

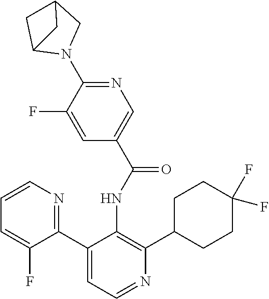 [SMILES: O=C(Nc1c(-c2ncccc2F)ccnc1C1CCC(F)(F)CC1)c1cnc(N2CC3CC2C3)c(F)c1]

To a mixture N-[2-(4,4-difluorocyclohexyl)-4-(3-fluoro-2-pyridyl)-3-pyridyl]-5,6-difluoro-pyridine-3-carboxamide (0.070 g, 0.16 mmol) in anhydrous dimethylformamide (1.6 mL) was added 2-azabicyclo[2.1.1]hexane hydrochloride (0.037 g, 0.32 mmol) and N,N-diisopropylethylamine (0.33 mL, 1.9 mmol). The solution was stirred at 50° C. for 3 h. The reaction mixture was diluted with ethyl acetate (10 mL) and washed with saturated ammonium chloride solution (2×20 mL). The organic layer was dried over anhydrous magnesium sulfate, filtered, and concentrated in vacuo. Purification of the residue by column chromatography, eluting with a gradient of 5 to 70% ethyl acetate in heptane, provided the title compound as an off-white solid (0.13 g, 48% yield): ¹H-NMR (400 MHz; DMSO-d₆) δ 9.91 (s, 1H), 8.62 (d, J=4.9 Hz, 1H), 8.46-8.44 (m, 1H), 8.32 (s, 1H), 7.82-7.77 (m, 1H), 7.67 (dd, J=13.7, 1.7 Hz, 1H), 7.49-7.43 (m, 2H), 4.85 (d, J=6.9 Hz, 1H), 3.55 (d, J=3.5 Hz, 2H), 3.20-3.13 (m, 1H), 2.95-2.92 (m, 1H), 2.13-2.08 (m, 2H), 1.97-1.82 (m, 8H), 1.36-1.30 (m, 2H); MS (ES+) m/z 512.2 (M+1).

In a similar manner as described in examples disclosed herein, utilizing the appropriately substituted starting materials and intermediates, the following compounds were prepared:

| Example | Structure | Amount | | |
|---|---|---|---|---|
| No. | Name | Yield | MS (ES+) m/z | ¹H NMR |
| 420 | 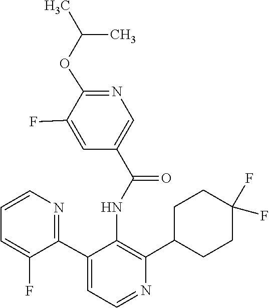 [SMILES: CC(C)Oc1ncc(C(=O)Nc2c(-c3ncccc3F)ccnc2C2CCC(F)(F)CC2)cc1F] N-(2′-(4,4- difluorocyclohexyl)-3- fluoro-[2,4′-bipyridin]-3′- yl)-5-fluoro-6-(isopropylamino) nicotinamide | 0.061 g 80% | 488.2 (M + 1) | (400 MHz, DMSO-d₆) δ 9.84 (s, 1H), 8.62 (d, J = 4.9 Hz, 1H), 8.45 (dd, J = 3.3, 1.3 Hz, 1H), 8.29 (s, 1H), 7.81-7.76 (m, 1H), 7.56 (dd, J = 12.3, 1.7 Hz, 1H), 7.49-7.42 (m, 2H), 7.04 (d, J = 7.5 Hz, 1H), 4.26 (dsextet, J = 13.7, 6.8 Hz, 1H), 3.20- 3.14 (m, 1H), 2.12-2.08 (m, 2H), 1.97-1.78 (m, 6H), 1.18 (d, J = 6.5 Hz, 6H) |

### Example 421

### Synthesis of N-(2′-(4,4-difluorocyclohexyl)-3,5-difluoro-[2,4′-bipyridin]-3′-yl)-2-isopropoxypyrimidine-5-carboxamide

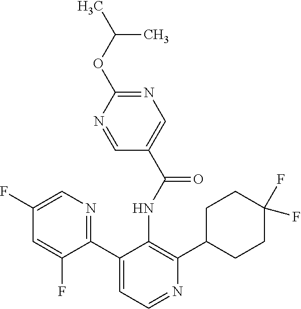 [SMILES: CC(C)Oc1ncc(C(=O)Nc2c(-c3ncc(F)cc3F)ccnc2C2CCC(F)(F)CC2)cn1]

### Step 1. Preparation of tert-butyl (2′-chloro-3,5-difluoro-[2,4′-bipyridin]-3′-yl)carbamate

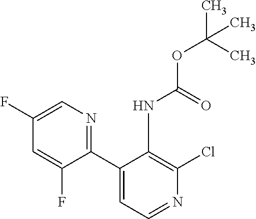 [SMILES: CC(C)(C)OC(=O)Nc1c(-c2ncc(F)cc2F)ccnc1Cl]

To a mixture of tert-butyl (2-chloro-4-iodopyridin-3-yl)carbamate (0.5 g, 1.4 mmol), 3,5-difluoro-2-tributylstannylpyridine (0.63 g, 1.6 mmol), and anhydrous dimethylformamide (3.5 mL) were sparged with nitrogen gas for 5 min. To the sparged mixture was added copper (1) iodide (0.027 g, 0.14 mmol) and tetrakis(triphenylphosphine)palladium(0) (0.098 g, 0.085 mmol). The reaction mixture was heated to 115° C. for 1 h. After cooling to ambient temperature, the reaction mixture was diluted with ethyl acetate (30 mL). The organic layer was washed with saturated ammonium chloride (2×20 mL), dried over anhydrous magnesium sulfate, filtered, and concentrated in vacuo. The residue was purified by column chromatography, eluting with a gradient of 0 to 70% of ethyl acetate in heptane, to provide the title compound as a yellow solid (0.32 g, 67% yield): MS (ES+) m/z 342.2 (M+1), 344.2 (M+1).

### Step 2. Preparation of tert-butyl (2′-(4,4-difluorocyclohexyl)-3,5-difluoro-[2,4′-bipyridin]-3′-yl)carbamate

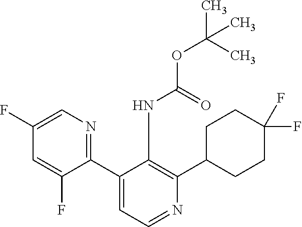 [SMILES: CC(C)(C)OC(=O)Nc1c(-c2ncc(F)cc2F)ccnc1C1CCC(F)(F)CC1]

To a mixture of (4,4″-di-t-butyl-2,2″-bipyridine)bis[3,5-difluoro-2-[5-trifluoromethyl-2-pyridinyl kN)phenyl-kCiridium(III) hexafluorophosphate (0.0061 g, 0.0054 mmol), dichloro(dimethoxyethane)nickel (0.012 g, 0.054 mmol), 4,4′-di-tert-butyl-2,2′-bipyridine (0.022 g, 0.081 mmol), tert-butyl (2′-chloro-3,5-difluoro-[2,4′-bipyridin]-3′-yl)carbamate (0.19 g, 0.54 mmol), cesium carbonate (0.32 g, 0.97 mmol) and 4,4-difluorocyclohexanecarboxylic acid (0.13 g, 0.81 mmol) was added N,N-dimethylformamide (13 mL). The mixture was purged with nitrogen for 10 s and sealed. The reaction mixture was then stirred in front of Kessil PR160L lights (440 nm) for 20 h. The reaction mixture was quenched with saturated aqueous sodium bicarbonate solution (10 mL) and extracted with ethyl acetate (3×20 mL). The combined organic extracts were washed with water (3×50 mL) and brine (50 mL), dried over anhydrous sodium sulfate, and concentrated in vacuo. Purification of the residue by column chromatography, eluting with 5 to 70% ethyl acetate in heptane, afforded the title compound as a colorless solid (0.13 g, 58% yield): MS (ES+) m/z 439.2 (M+1).

### Step 3. Preparation of 2′-(4,4-difluorocyclohexyl)-3,5-difluoro-[2,4′-bipyridin]-3′-amine

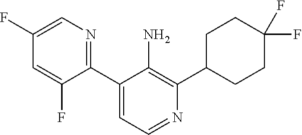 [SMILES: Nc1c(-c2ncc(F)cc2F)ccnc1C1CCC(F)(F)CC1]

A flask containing tert-butyl (2′-(4,4-difluorocyclohexyl)-3,5-difluoro-[2,4′-bipyridin]-3′-yl)carbamate (0.13 g, 0.31 mmol) in 1,4-dioxane (0.62 mL) was charged with 4M hydrogen chloride in 1,4-dioxane (0.78 mL). The reaction vessel was sealed and heated to 45° C. for 2 h. The reaction mixture was neutralized with dropwise addition of N,N-diisopropylethylamine until pH becomes neutral. The organic layer was separated and washed with brine (10 mL), dried over anhydrous magnesium sulfate, filtered, and concentrated in vacuo. The residue was purified by column chromatography, eluting with a gradient of 5 to 80% of ethyl acetate in heptane, to provide the title compound as a yellow solid (0.084 g, 83% yield): MS (ES+) m/z 326.3 (M+1).

### Step 4. Preparation of 2-chloro-N-(2′-(4,4-difluorocyclohexyl)-3,5-difluoro-[2,4′-bipyridin]-3′-yl)pyrimidine-5-carboxamide

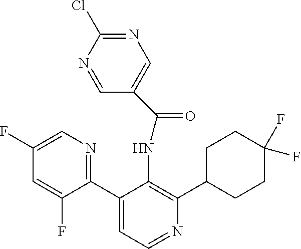 [SMILES: O=C(Nc1c(-c2ncc(F)cc2F)ccnc1C1CCC(F)(F)CC1)c1cnc(Cl)nc1]

A mixture containing 2′-(4,4-difluorocyclohexyl)-3,5-difluoro-[2,4′-bipyridin]-3′-amine (0.081 g, 0.25 mmol), 2-chloro-1-methylpyridinium iodide (0.19 g, 0.75 mmol), 2-chloropyrimidine-5-carboxylic acid (0.059 g, 0.37 mmol), and anhydrous tetrahydrofuran (2.5 mL) was charged with N,N-diisopropylethylamine (0.26 mL, 1.5 mmol). The reaction vessel was sealed and stirred at 68° C. for 20 h. The reaction mixture was diluted with ethyl acetate (20 mL). The organic layer was washed with 1M sodium hydroxide (20 mL), saturated ammonium chloride (2×30 mL), and water (30 mL), then dried over anhydrous magnesium sulfate, filtered, and concentrated in vacuo. The resulting solid was used in the next step without further purification (0.12 g, quant. yield, crude): MS (ES+) m/z 466.3 (M+1), 468.4 (M+1).

### Step 5. Preparation of N-(2′-(4,4-difluorocyclohexyl)-3,5-difluoro-[2,4′-bipyridin]-3′-yl)-2-isopropoxypyrimidine-5-carboxamide

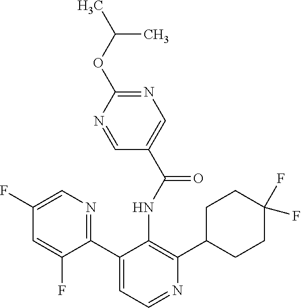 [SMILES: CC(C)Oc1ncc(C(=O)Nc2c(-c3ncc(F)cc3F)ccnc2C2CCC(F)(F)CC2)cn1]

To a mixture of 2-chloro-N-(2′-(4,4-difluorocyclohexyl)-3,5-difluoro-[2,4′-bipyridin]-3′-yl)pyrimidine-5-carboxamide (0.12 g, 0.25 mmol) in anhydrous dimethylformamide (2.5 mL) was added 2-propanol (2.5 mL, 0.25 mmol) and 60% sodium hydride dispersion in mineral oil (0.099 g, 2.5 mmol). The solution was stirred at 45° C. for 3 h. The reaction mixture was diluted with ethyl acetate (10 mL) and washed with saturated ammonium chloride solution (2×20 mL). The organic layer was dried over anhydrous magnesium sulfate, filtered, and concentrated in vacuo. Purification of the residue by preparative reverse phase HPLC, using 5 to 85% acetonitrile in water containing 0.5% of formic acid as eluent, afforded the title compound as a colorless solid (0.012 g, 10% yield): ¹H-NMR (400 MHz; DMSO-d₆) δ 10.25 (s, 1H), 8.87 (s, 2H), 8.65 (d, J=4.9 Hz, 1H), 8.57 (d, J=2.2 Hz, 1H), 8.10-8.05 (m, 1H), 7.45 (d, J=4.8 Hz, 1H), 5.27 (7, J=6.2 Hz, 1H), 3.23-3.17 (m, 1H), 2.13-2.06 (m, 2H), 2.01-1.84 (m, 6H), 1.34 (d, J=6.2 Hz, 6H). MS (ES+) m/z 490.2 (M+1)

In a similar manner as described in examples disclosed herein, utilizing the appropriately substituted starting materials and intermediates, the following compounds were prepared:

| Example | Structure | Amount | | |
|---|---|---|---|---|
| No. | Name | Yield | MS (ES+) m/z | ¹H NMR |
| 422 | 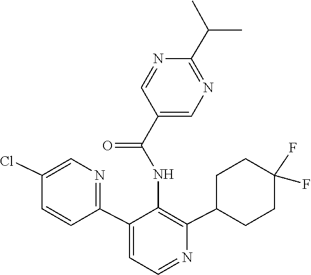 [SMILES: CC(C)c1ncc(C(=O)Nc2c(-c3ccc(Cl)cn3)ccnc2C2CCC(F)(F)CC2)cn1] N-(5-chloro-2′-(4,4- difluorocyclohexyl)-[2,4′- bipyridin]-3′-yl)-2- isopropylpyrimidine-5- carboxamide | 0.051 g 38% | 472.2 (M + 1) | (400 MHz, MeOD) δ 9.10 (s, 2H), 8.65 (d, J = 5.0 Hz, 1H), 8.63 (d, J = 2.4 Hz, 1H), 7.94 (dd, J = 8.5, 2.5 Hz, 1H), 7.69-7.66 (m, 1H), 7.54 (d, J = 5.0 Hz, 1H), 3.28 (dd, J = 13.8, 6.9 Hz, 1H), 3.24-3.17 (m, 1H), 2.20-2.14 (m, 2H), 2.10-2.03 (m, 2H), 1.96-1.93 (m, 3H), 1.90-1.81 (m, 1H), 1.38 (d, J = 6.9 Hz, 6H) |
| 423 | 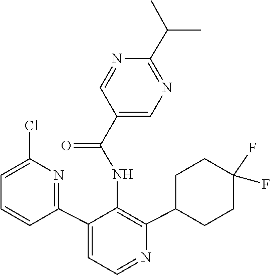 [SMILES: CC(C)c1ncc(C(=O)Nc2c(-c3cccc(Cl)n3)ccnc2C2CCC(F)(F)CC2)cn1] N-(6-chloro-2′-(4,4- difluorocyclohexyl)-[2,4′- bipyridin]-3′-yl)-2- isopropylpyrimidine-5- carboxamide | 0.020 g 14% | 472.2 (M + 1), 474.2 (M + 1) | (400 MHz, DMSO-d₆) δ 10.45 (s, 1H), 9.09 (s, 2H), 8.67 (d, J = 4.9 Hz, 1H), 7.95 (t, J = 7.8 Hz, 1H), 7.67 (d, J = 7.6 Hz, 1H), 7.52 (dd, J = 6.4, 4.3 Hz, 2H), 3.27-3.17 (m, 2H), 2.12-2.06 (m, 2H), 2.01-1.81 (m, 6H), 1.30 (d, J = 6.9 Hz, 6H) |
| 424 | 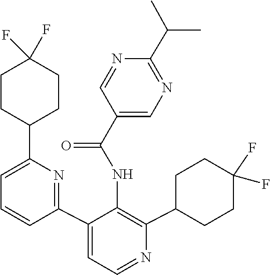 [SMILES: CC(C)c1ncc(C(=O)Nc2c(-c3cccc(C4CCC(F)(F)CC4)n3)ccnc2C2CCC(F)(F)CC2)cn1] N-(2′,6-bis(4,4- difluorocyclohexyl)-[2,4′- bipyridin]-3′-yl)-2- isopropylpyrimidine-5- carboxamide | 0.0048 g 2.7% | 556.4 (M + 1) | (400 MHz, DMSO-d₆) δ 10.39 (s, 1H), 9.10 (s, 2H), 8.64 (d, J = 4.9 Hz, 1H), 7.84-7.80 (m, 1H), 7.51-7.49 (m, 2H), 7.28 (d, J = 7.8 Hz, 1H), 3.24-3.18 (m, 2H), 2.78-2.71 (m, 1H), 2.12-2.05 (m, 2H), 2.03-1.85 (m, 9H), 1.81-1.57 (m, 5H), 1.29 (d, J = 6.8 Hz, 6H) |

In a similar manner as described in examples disclosed herein, utilizing the appropriately substituted starting materials and intermediates, the following compounds were prepared:

| Example | Structure | Amount | | |
|---|---|---|---|---|
| No. | Name | Yield | MS (ES+) m/z | ¹H NMR |
| 425 | 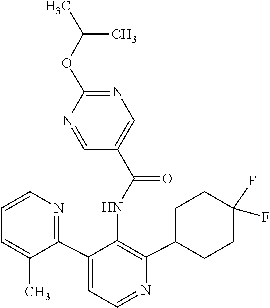 [SMILES: Cc1cccnc1-c1ccnc(C2CCC(F)(F)CC2)c1NC(=O)c1cnc(OC(C)C)nc1] N-(2′-(4,4- difluorocyclohexyl)-3- methyl-[2,4′-bipyridin]-3′- yl)-2-isopropoxypyrimidine-5- carboxamide | 0.022 g 46% | 468.2 (M + 1) | (400 MHz, DMSO-d₆) δ 10.09 (s, 1H), 8.72 (s, 2H), 8.60 (d, J = 4.9 Hz, 1H), 8.39 (d, J = 4.4 Hz, 1H), 7.68 (d, J = 7.7 Hz, 1H), 7.33 (d, J = 4.8 Hz, 1H), 7.25 (dd, J = 7.7, 4.8 Hz, 1H), 5.23 (7, J = 6.2 Hz, 1H), 3.18-3.12 (m, 1H), 2.16 (s, 3H), 2.13-2.06 (m, 2H), 2.00- 1.82 (m, 6H), 1.32 (d, J = 6.2 Hz, 6H) |
| 426 | 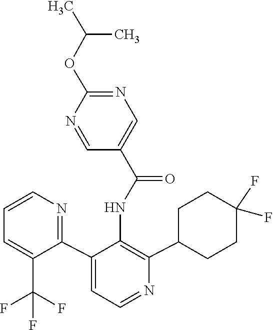 [SMILES: CC(C)Oc1ncc(C(=O)Nc2c(-c3ncccc3C(F)(F)F)ccnc2C2CCC(F)(F)CC2)cn1] N-(2′-(4,4- difluorocyclohexyl)-3- (trifluoromethyl)-[2,4′- bipyridin]-3′-yl)-2- isopropoxypyrimidine-5- carboxamide | 0.033 g 61% | 522.2 (M + 1) | (400 MHz, DMSO-d₆) δ 9.98 (s, 1H), 8.84 (d, J = 4.5 Hz, 1H), 8.71 (s, 2H), 8.63 (d, J = 4.8 Hz, 1H), 8.30 (d, J = 8.0 Hz, 1H), 7.66-7.62 (m, 1H), 7.31 (d, J = 4.8 Hz, 1H), 5.24 (dquintet, J = 12.3, 6.1 Hz, 1H), 3.16- 3.11 (m, 1H), 2.16-2.06 (m, 2H), 2.00-1.79 (m, 6H), 1.32 (d, J = 6.1 Hz, 6H) |
| 427 | 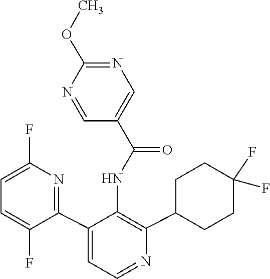 [SMILES: COc1ncc(C(=O)Nc2c(-c3nc(F)ccc3F)ccnc2C2CCC(F)(F)CC2)cn1] N-(2′-(4,4- difluorocyclohexyl)-3,6- difluoro-[2,4′-bipyridin]- 3′-yl)-2-methoxypyrimidine-5- carboxamide | 0.055 g 46% | 462.2 (M + 1) | (400 MHz, DMSO-d₆) δ 10.36 (s, 1H), 8.92 (s, 2H), 8.69 (d, J = 4.9 Hz, 1H), 8.35 (s, 1H), 7.46 (d, J = 4.9 Hz, 1H), 7.31 (dd, J = 4.4, 2.7 Hz, 1H), 3.99 (s, 3H), 3.27- 3.21 (m, 1H), 2.13-2.07 (m, 2H), 1.99-1.83 (m, 6H) |

### Example 428

### Synthesis of 2-(tert-butyl)-N-(2′-(4,4-difluorocyclohexyl)-2,5-difluoro-[3,4′-bipyridin]-3′-yl)pyrimidine-5-carboxamide

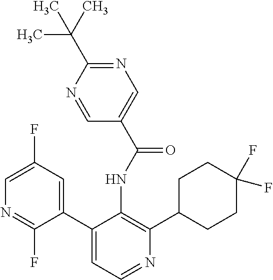 [SMILES: CC(C)(C)c1ncc(C(=O)Nc2c(-c3cc(F)cnc3F)ccnc2C2CCC(F)(F)CC2)cn1]

### Step 1. Preparation of tert-butyl (2′-chloro-2,5-difluoro-[3,4′-bipyridin]-3′-yl)carbamate

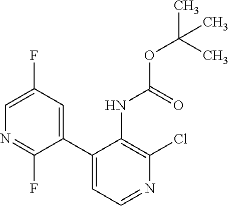 [SMILES: CC(C)(C)OC(=O)Nc1c(-c2cc(F)cnc2F)ccnc1Cl]

A mixture of (2,5-difluoropyridin-3-yl)boronic acid (0.39 g, 2.5 mmol) in 1,4-dioxane (8.1 mL) and water (0.90 mL) was degassed with nitrogen for 10 minutes. To the reaction mixture was added potassium carbonate (0.94 g, 6.8 mmol), tert-butyl N-(2-chloro-4-iodo-3-pyridyl)carbamate (0.80 g, 2.3 mmol), [1,1′-bis(diphenylphosphino)ferrocene]dichloropalladium(II), complex with dichloromethane (1:1) (0.19 g, 0.23 mmol), and the reaction mixture was stirred at 90° C. for 3 h. After cooling to ambient temperature, the mixture was filtered through a bed of Celite, and the filter pad was washed with ethyl acetate (2×20 mL). The combined filtrate was washed with saturated ammonium chloride (2×25 mL) and concentrated in vacuo. The residue was purified by column chromatography, eluting with a gradient of 0 to 40% ethyl acetate in heptane, to afford the title compound as a yellow solid (0.39 g, 51% yield): MS (ES+) m/z 342.2 (M+1), 344.2 (M+1)

### Step 2. Preparation of tert-butyl (2′-(4,4-difluorocyclohexyl)-2,5-difluoro-[3,4′-bipyridin]-3′-yl)carbamate

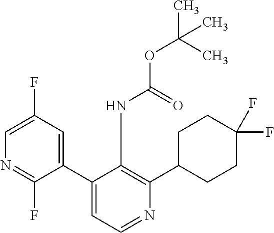 [SMILES: CC(C)(C)OC(=O)Nc1c(-c2cc(F)cnc2F)ccnc1C1CCC(F)(F)CC1]

To a mixture of (4,4″-di-t-butyl-2,2″-bipyridine)bis[3,5-difluoro-2-[5-trifluoromethyl-2-pyridinyl kN)phenyl-kC]iridium(III) hexafluorophosphate (0.013 g, 0.011 mmol), dichloro(dimethoxyethane)nickel (0.025 g, 0.11 mmol), 4,4′-di-tert-butyl-2,2′-bipyridine (0.045 g, 0.17 mmol), tert-butyl (2′-chloro-2,5-difluoro-[3,4′-bipyridin]-3′-yl)carbamate (0.39 g, 1.1 mmol), cesium carbonate (0.66 g, 2.0 mmol) and 4,4-difluorocyclohexanecarboxylic acid (0.28 g, 1.7 mmol) was added N,N-dimethylformamide (23 mL). The mixture was purged with nitrogen for 10 s and sealed. The reaction mixture was then stirred in front of Kessil PR160L lights (440 nm) for 20 h. The reaction mixture was quenched with saturated aqueous sodium bicarbonate solution (30 mL) and extracted with ethyl acetate (3×30 mL). The combined organic extracts were washed with water (2×50 mL) and brine (50 mL), dried over anhydrous sodium sulfate, and concentrated in vacuo. Purification of the residue by a column chromatography, eluting with 5-90% ethyl acetate in heptane, afforded the title compound as a light-yellow solid (0.32 g, 67% yield): MS (ES+) m/z 426.4 (M+1)

### Step 3. Preparation of 2′-(4,4-difluorocyclohexyl)-2,5-difluoro-[3,4′-bipyridin]-3′-amine

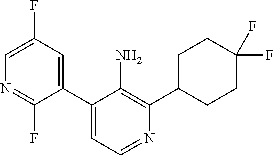 [SMILES: Nc1c(-c2cc(F)cnc2F)ccnc1C1CCC(F)(F)CC1]

To a solution of tert-butyl (2′-(4,4-difluorocyclohexyl)-2,5-difluoro-[3,4′-bipyridin]-3′-yl)carbamate (0.32 g, 0.75 mmol) in 1,4-dioxane (3.0 mL) was added 4M hydrogen chloride in 1,4-dioxane (3.8 mL). The reaction vessel was sealed and heated to 45° C. for 2 h. The reaction mixture was neutralized with dropwise addition of N,N-diisopropylethylamine until the pH was neutral. The organic layer was separated and washed with brine (10 mL), dried over anhydrous magnesium sulfate, filtered, and concentrated in vacuo. The residue was purified by column chromatography, eluting with a gradient of 5 to 80% of ethyl acetate in heptane, to provide the title compound as a yellow solid (0.23 g, 92% yield): MS (ES+) m/z 326.2 (M+1).

### Step 4. Preparation of 2-(tert-butyl)-N-(2′-(4,4-difluorocyclohexyl)-2,5-difluoro-[3,4′-bipyridin]-3′-yl)pyrimidine-5-carboxamide

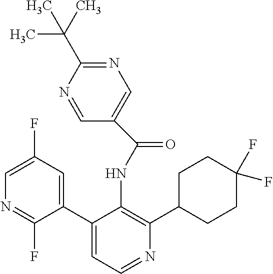 [SMILES: CC(C)(C)c1ncc(C(=O)Nc2c(-c3cc(F)cnc3F)ccnc2C2CCC(F)(F)CC2)cn1]

A mixture containing 2′-(4,4-difluorocyclohexyl)-2,5-difluoro-[3,4′-bipyridin]-3′-amine (0.11 g, 0.34 mmol), 2-chloro-1-methylpyridinium iodide (0.26 g, 1.0 mmol), 2-tert-butylpyrimidine-5-carboxylic acid (0.093 g, 0.52 mmol), and anhydrous tetrahydrofuran (3.4 mL) was charged with N,N-diisopropylethylamine (0.36 mL, 2.1 mmol). The reaction vessel was sealed and stirred at 68° C. for 20 h. The reaction mixture was diluted with ethyl acetate (20 mL). The organic layer was washed with 1M sodium hydroxide (20 mL), saturated ammonium chloride (2×30 mL), and water (30 mL), then dried over anhydrous magnesium sulfate, filtered, and concentrated in vacuo. Purification of the residue by column chromatography, eluting with a gradient of 5 to 95% ethyl acetate in heptane, provided the title compound as a colorless solid (0.14 g, 76% yield): ¹H-NMR (400 MHz; DMSO-d₆) δ 10.44 (s, 1H), 9.00 (s, 2H), 8.67 (d, J=4.9 Hz, 1H), 8.29 (dd, J=2.8, 1.6 Hz, 1H), 8.00 (td, J=7.6, 3.0 Hz, 1H), 7.46 (d, J=4.9 Hz, 1H), 3.26-3.23 (m, 1H), 2.08-1.84 (m, 8H), 1.37 (s, 9H); MS (ES+) m/z 488.3 (M+1).

In a similar manner as described in examples disclosed herein, utilizing the appropriately substituted starting materials and intermediates, the following compounds were prepared:

| Example | Structure | Amount | | |
|---|---|---|---|---|
| No. | Name | Yield | MS (ES+) m/z | ¹H NMR |
| 429 | 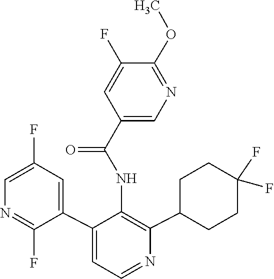 [SMILES: COc1ncc(C(=O)Nc2c(-c3cc(F)cnc3F)ccnc2C2CCC(F)(F)CC2)cc1F] N-(2′-(4,4- difluorocyclohexyl)-2,5- difluoro-[3,4′-bipyridin]- 3′-yl)-5-fluoro-6- methoxynicotinamide | 0.077 g 44% | 479.2 (M + 1) | (400 MHz, DMSO-d₆) δ 10.24 (s, 1H), 8.66 (d, J = 4.9 Hz, 1H), 8.42 (d, J = 1.7 Hz, 1H), 8.27 (t, J = 1.4 Hz, 1H), 7.99-7.94 (m, 2H), 7.45 (d, J = 4.9 Hz, 1H), 4.01 (s, 3H), 3.23- 3.17 (m, 1H), 2.13-2.05 (m, 2H), 2.00-1.82 (m, 6H) |
| 430 | 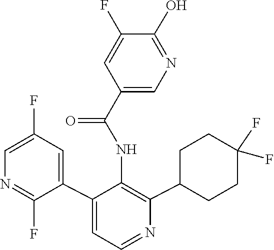 [SMILES: O=C(Nc1c(-c2cc(F)cnc2F)ccnc1C1CCC(F)(F)CC1)c1cnc(O)c(F)c1] N-(2′-(4,4- difluorocyclohexyl)-2,5- difluoro-[3,4′-bipyridin]- 3′-yl)-5-fluoro-6- hydroxynicotinamide | 0.0078 g 4.8% | 465.2 (M + 1) | (400 MHz, DMSO-d₆) δ 10.00 (s, 1H), 8.63 (d, J = 4.9 Hz, 1H), 8.27 (d, J = 0.9 Hz, 1H), 7.94 (td, J = 7.6, 2.8 Hz, 1H), 7.88 (s, 1H), 7.65 (d, J = 11.5 Hz, 1H), 7.42 (d, J = 4.9 Hz, 1H), 3.19-3.14 (m, 1H), 2.13-2.07 (m, 2H), 1.99-1.76 (m, 6H) |

### Example 431

### Synthesis of 2-(tert-butyl)-N-(2′-(4,4-difluorocyclohexyl)-5-fluoro-2-methoxy-[3,4′-bipyridin]-3′-yl)pyrimidine-5-carboxamide

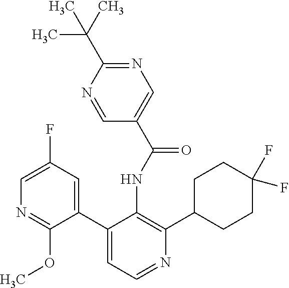 [SMILES: COc1ncc(F)cc1-c1ccnc(C2CCC(F)(F)CC2)c1NC(=O)c1cnc(C(C)(C)C)nc1]

### Step 1. Preparation of 2-(tert-butyl)-N-(2′-(4,4-difluorocyclohexyl)-5-fluoro-2-methoxy-[3,4′-bipyridin]-3′-yl)pyrimidine-5-carboxamide

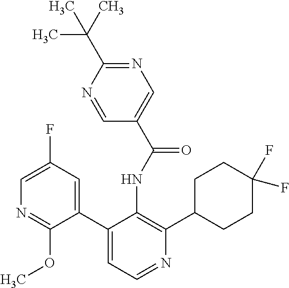 [SMILES: COc1ncc(F)cc1-c1ccnc(C2CCC(F)(F)CC2)c1NC(=O)c1cnc(C(C)(C)C)nc1]

To a mixture of N-(2′-(4,4-difluorocyclohexyl)-3-fluoro-[2,4′-bipyridin]-3′-yl)-6-fluoronicotinamide (0.070 g, 0.14 mmol) in anhydrous dimethylsulfoxide (1.3 mL) was added methanol (1.3 mL, 0.14 mmol) and 60% sodium hydride dispersion in mineral oil (0.057 g, 1.4 mmol). The solution was stirred at 65° C. for 18 h. The reaction mixture was diluted with ethyl acetate (10 mL) and washed with saturated ammonium chloride solution (2×20 mL). The organic layer was dried over anhydrous magnesium sulfate, filtered, and concentrated in vacuo. Purification of the residue by column chromatography, eluting with a gradient of 5 to 80% ethyl acetate in heptane, provided the title compound as a colorless solid (0.088 g, 55% yield): ¹H-NMR (400 MHz; DMSO-d₆) δ 10.21 (s, 1H), 8.96 (s, 2H), 8.60 (d, J=4.9 Hz, 1H), 8.18 (d, J=3.0 Hz, 1H), 7.66 (dd, J=8.4, 2.8 Hz, 1H), 7.35 (d, J=4.9 Hz, 1H), 3.77 (s, 3H), 3.21-3.14 (m, 1H), 2.12-1.80 (m, 9H), 1.37 (s, 9H); MS (ES+) m/z 500.4 (M+1)

### Example 432

### Synthesis of 2-(tert-butyl)-N-(2′-(4,4-difluorocyclohexyl)-3,6-difluoro-[2,4′-bipyridin]-3′-yl)pyrimidine-5-carboxamide

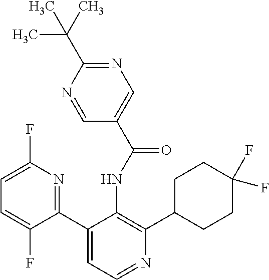 [SMILES: CC(C)(C)c1ncc(C(=O)Nc2c(-c3nc(F)ccc3F)ccnc2C2CCC(F)(F)CC2)cn1]

### Step 1. Preparation of tert-butyl (2′-chloro-3,6-difluoro-[2,4′-bipyridin]-3′-yl)carbamate

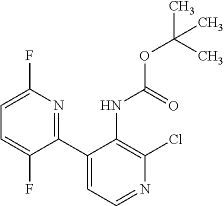 [SMILES: CC(C)(C)OC(=O)Nc1c(-c2nc(F)ccc2F)ccnc1Cl]

To a solution of tert-butyl (2-chloro-4-iodopyridin-3-yl)carbamate (0.43 g, 1.2 mmol) in anhydrous DMF (3.0 mL) was added tributyl-(3,6-difluoro-2-pyridyl)stannane (0.54 g. 1.3 mmol), copper(I) iodide (0.023 g, 0.12 mmol) and tetrakis(triphenylphosphine)palladium(0) (0.084 g. 0.073 mmol). The reaction mixture was degassed with nitrogen for 10 minutes and stirred at 115° C. for 3 h. After cooling to ambient temperature, the mixture was filtered through a bed of Celite and the filter pad was washed with ethyl acetate (2×20 mL). The filtrate was washed with saturated ammonium chloride (2×25 mL) and water (25 mL), then concentrated in vacuo. The residue was purified by column chromatography, eluting with a gradient of 5 to 40% ethyl acetate in heptane, to afford the title compound as a yellow solid (0.35 g, 84% yield): MS (ES+) m/z 342.2 (M+1), 344.2 (M+1).

### Step 2. Preparation of tert-butyl (2′-(4,4-difluorocyclohexyl)-3,6-difluoro-[2,4′-bipyridin]-3′-yl)carbamate

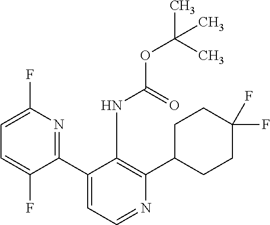 [SMILES: CC(C)(C)OC(=O)Nc1c(-c2nc(F)ccc2F)ccnc1C1CCC(F)(F)CC1]

To a mixture of (4,4″-di-t-butyl-2,2″-bipyridine)bis[3,5-difluoro-2-[5-trifluoromethyl-2-pyridinyl kN)phenyl-kC} iridium(III) hexafluorophosphate (0.0072 g, 0.0069 mmol), dichloro(dimethoxyethane)nickel (0.015 g, 0.069 mmol), 4,4′-di-tert-butyl-2,2′-bipyridine (0.028 g, 0.10 mmol), tert-butyl (2′-chloro-3,6-difluoro-[2,4′-bipyridin]-3′-yl)carbamate (0.24 g, 0.69 mmol), cesium carbonate (0.40 g, 1.2 mmol) and 4,4-difluorocyclohexanecarboxylic acid (0.17 g, 1.0 mmol) was added N,N-dimethylformamide (14 mL). The mixture was purged with nitrogen for 10 s and sealed. The reaction mixture was then stirred in front of Kessil PR160L lights (440 nm) for 20 h. The reaction mixture was quenched with saturated aqueous sodium bicarbonate solution (30 mL) and extracted with ethyl acetate (3×30 mL). The combined organic extracts were washed with water (2×50 mL) and brine (50 mL), dried over anhydrous sodium sulfate, and concentrated in vacuo. The residue was used in the next step without further purification (0.44 g, quant. yield, crude): MS (ES+) m/z 426.4 (M+1).

### Step 3. Preparation of 2′-(4,4-difluorocyclohexyl)-3,6-difluoro-[2,4′-bipyridin]-3′-amine

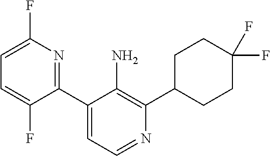 [SMILES: Nc1c(-c2nc(F)ccc2F)ccnc1C1CCC(F)(F)CC1]

A mixture containing tert-butyl (2′-(4,4-difluorocyclohexyl)-3,6-difluoro-[2,4′-bipyridin]-3′-yl)carbamate (0.44 g, 1.0 mmol) in 1,4-dioxane (2.0 mL) was charged with 4M hydrogen chloride in 1,4-dioxane (2.6 mL). The reaction vessel was sealed and heated to 45° C. for 2 h. The reaction mixture was neutralized with dropwise addition of N,N-diisopropylethylamine until pH becomes neutral. The organic layer was separated and washed with brine (10 mL), dried over anhydrous magnesium sulfate, filtered, and concentrated in vacuo. The residue was purified by column chromatography, eluting with a gradient of 10 to 70% of ethyl acetate in heptane, to provide the title compound as a yellow solid (0.17 g, 50% yield): MS (ES+) m/z 326.3 (M+1).

### Step 4. Preparation of 2-(tert-butyl)-N-(2′-(4,4-difluorocyclohexyl)-2,5-difluoro-[3,4′-bipyridin]-3′-yl)pyrimidine-5-carboxamide

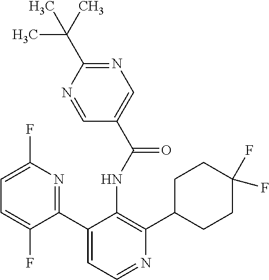 [SMILES: CC(C)(C)c1ncc(C(=O)Nc2c(-c3nc(F)ccc3F)ccnc2C2CCC(F)(F)CC2)cn1]

A mixture containing 2-(4,4-difluorocyclohexyl)-4-(3,6-difluoro-2-pyridyl)pyridin-3-amine (0.10 g, 0.31 mmol), 2-chloro-1-methylpyridinium iodide (0.24 g, 0.92 mmol), 2-tert-butylpyrimidine-5-carboxylic acid (0.083 g, 0.46 mmol), and anhydrous tetrahydrofuran (3.6 mL) was charged with N,N-diisopropylethylamine (0.32 mL, 1.8 mmol). The reaction vessel was sealed and stirred at 68° C. for 20 h. The reaction mixture was diluted with ethyl acetate (20 mL). The organic layer was washed with 1M sodium hydroxide (20 mL), saturated ammonium chloride (2×30 mL), and water (30 mL), then it was dried over anhydrous magnesium sulfate, filtered, and concentrated in vacuo. Purification of the residue by column chromatography, eluting with a gradient of 5 to 95% ethyl acetate in heptane, provided the title compound as a colorless solid (0.14 g, 76% yield): ¹H-NMR (400 MHz; MeOD) δ 9.02 (s, 2H), 8.70 (d, J=4.9 Hz, 1H), 8.20 (d, J=1.1 Hz, 1H), 7.44 (d, J=4.9 Hz, 1H), 7.16 (dd, J=4.5, 2.8 Hz, 1H), 3.24-3.18 (m, 1H), 2.20-2.14 (m, 2H), 2.11-2.04 (m, 2H), 1.97-1.93 (m, 2H), 1.90-1.80 (m, 2H), 1.43 (s, 9H); MS (ES+) m/z 488.3 (M+1) Example 433

### Synthesis of N-(2-(4,4-difluorocyclohexyl)-4-(2,5-difluorophenyl)pyridin-3-yl)-3-methoxybicyclo[1.1.1]pentane-1-carboxamide

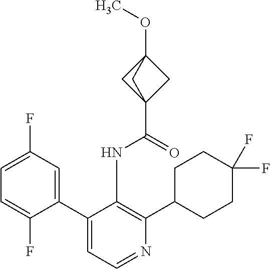 [SMILES: COC12CC(C(=O)Nc3c(-c4cc(F)ccc4F)ccnc3C3CCC(F)(F)CC3)(C1)C2]

### Step 1. Preparation of N-(2-(4,4-difluorocyclohexyl)-4-(2,5-difluorophenyl)pyridin-3-yl)-3-methoxybicyclo[1.1.1]pentane-1-carboxamide

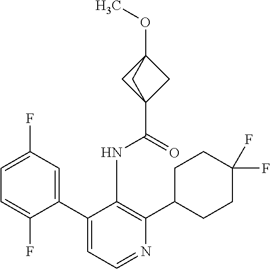 [SMILES: COC12CC(C(=O)Nc3c(-c4cc(F)ccc4F)ccnc3C3CCC(F)(F)CC3)(C1)C2]

To a mixture of 2-(4,4-difluorocyclohexyl)-4-(2,5-difluorophenyl)pyridin-3-amine (0.080 g, 0.25 mmol), 2-chloro-1-methylpyridinium iodide (0.19 g, 0.74 mmol), 3-methoxybicyclo [1.1.1]pentane-1-carboxylic acid (0.053 g, 0.37 mmol), and anhydrous tetrahydrofuran (2.5 mL) was added N,N-diisopropylethylamine (0.26 mL, 1.5 mmol). The reaction vessel was sealed and stirred at 68° C. for 20 h. The reaction mixture was diluted with ethyl acetate (20 mL). The organic layer was washed with 1 M sodium hydroxide (20 mL, saturated ammonium chloride (2×30 mL), and water (30 mL, and then it was dried over anhydrous magnesium sulfate, filtered, and concentrated in vacuo. The residue was purified by column chromatography, eluting with a gradient of 5 to 70% of ethyl acetate in heptane, to provide the title compound as a colorless solid (0.072 g, 64% yield): ¹H-NMR (400 MHz; DMSO-d₆) δ 9.42 (, 1H), 8.55 (d, J=4.9 Hz, 1H), 7.41-7.29 (m, 3H), 7.15 (ddd, J=8.9, 5.7, 3.1 Hz, 1H), 3.18 (, 3H), 3.06-3.01 (m, 1H), 2.18-2.12 (m, 2H), 1.96 (s, 6H), 1.93-1.79 (n, 6H); MS (ES+) m/z 449.2 (M+1)

In a similar manner as described in examples disclosed herein, utilizing the appropriately substituted starting materials and intermediates, the following compounds were prepared:

| Example | Structure | Amount | | |
|---|---|---|---|---|
| No. | Name | Yield | MS (ES+) m/z | ¹H NMR |
| 434 | 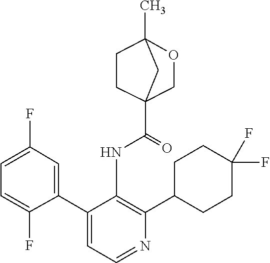 [SMILES: CC12CCC(C(=O)Nc3c(-c4cc(F)ccc4F)ccnc3C3CCC(F)(F)CC3)(CO1)C2] N-(2-(4,4- difluorocyclohexyl)-4- (2,5-difluorophenyl)pyridin-3- yl)-1-methyl-2- oxabicyclo[2.2.1] heptane-4-carboxamide | 0.087 g 61% | 463.2 (M + 1) | (400 MHz, DMSO-d₆) δ 9.35 (s, 1H), 8.56 (d, J = 4.9 Hz, 1H), 7.41-7.30 (m, 3H), 7.14 (ddd, J = 8.8, 5.7, 3.1 Hz, 1H), 3.58 (m, 2H), 3.07-3.02 (m, 1H), 2.13 (m, 2H), 1.94-1.54 (m, 12H), 1.29 (s, 3H) |
| 435 | 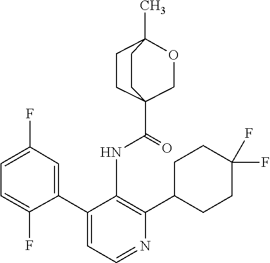 [SMILES: CC12CCC(C(=O)Nc3c(-c4cc(F)ccc4F)ccnc3C3CCC(F)(F)CC3)(CC1)CO2] N-(2-(4,4- difluorocyclohexyl)-4- (2,5-difluorophenyl)pyridin-3- yl)-1-methyl-2- oxabicyclo[2.2.2]octane- 4-carboxamide | 0.091 g 61% | 477.2 (M + 1) | (400 MHz, DMSO-d₆) δ 9.21 (s, 1H), 8.54 (d, J = 4.9 Hz, 1H), 7.35 (ddt, J = 20.6, 12.7, 4.3 Hz, 3H), 7.10 (ddd, J = 8.8, 5.7, 3.1 Hz, 1H), 3.63 (s, 2H), 3.02-2.98 (m, 1H), 2.14-2.10 (m, 2H), 1.96-1.55 (m, 14H), 0.99 (s, 3H) |
| 436 | 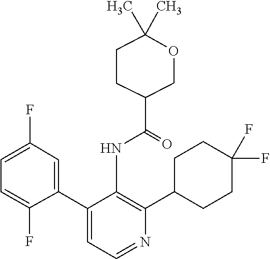 [SMILES: CC1(C)CCC(C(=O)Nc2c(-c3cc(F)ccc3F)ccnc2C2CCC(F)(F)CC2)CO1] difluorocyclohexyl)-4-(2,5- difluorophenyl)pyridin-3-yl)-6,6- dimethyltetrahydro-2/7- pyran-3-carboxamide | 0.069 g 60% | 465.2 (M + 1) | (400 MHz, DMSO-d₆) δ 9.58 (s, 1H), 8.54 (d, J = 4.9 Hz, 1H), 7.36 (ddt, J = 18.2, 8.7, 4.2 Hz, 2H), 7.29 (d, J = 4.9 Hz, 1H), 7.14- 7.10 (m, 1H), 3.51-3.40 (m, 2H), 3.10-3.02 (m, 1H), 2.39 (tt, J = 10.2, 5.0 Hz, 1H), 2.15-2.10 (m, 2H), 1.95-1.75 (m, 6H), 1.67-1.47 (m, 3H), 1.34-1.22 (m, 1H), 1.10 (d, J = 6.7 Hz, 6H) |

### Example 437

### Synthesis of N-(4-(4,4-difluorocyclohexyl)-6-(2,5-difluorophenyl)pyrimidin-5-yl)-5-fluoro-6-methoxynicotinamide

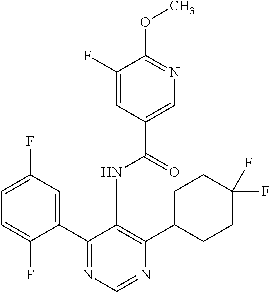 [SMILES: COc1ncc(C(=O)Nc2c(-c3cc(F)ccc3F)ncnc2C2CCC(F)(F)CC2)cc1F]

### Step 1. Preparation of 4-chloro-6-(2,5-difluorophenyl)pyrimidin-5-amine

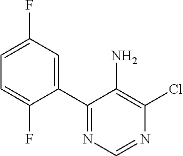 [SMILES: Nc1c(Cl)ncnc1-c1cc(F)ccc1F]

To 5-amino-4,6-dichloropyrimidine (3.0 g, 18 mmol) was added 1,4-dioxane (82 mL) and water (9.2 mL) and the mixture was sparged with nitrogen for 10 min. To the mixture was added 2.5-difluorophenylboronc acid (2.9 g, 18 mmol), potassium carbonate (7.59 g, 54. mmol), and [1,1′-Bis(diphenylphosphino)ferrocene]dichloropalladium(II), complex with dichloromethane (1.55 g, 1.83 mmol), and the solution was sparged with nitrogen for 2 min. The flask was sealed under a nitrogen atmosphere and heated to 68° C. for 4 h. The reaction mixture was cooled to ambient temperature and diluted with ethyl acetate (200 mL). The organic layer was washed with saturated ammonium chloride (2×100 mL), water (100 mL), dried over anhydrous magnesium sulfate, filtered, and concentrated in vacuo. Purification of the residue by column chromatography, eluting with 5-80% ethyl acetate in heptane, afforded the title compound as a light-yellow solid (2.7 g, 61% yield): MS (ES+) m/z 242.2 (M+1), 244.2 (M+1).

### Step 2. Preparation of 4-(4,4-difluorocyclohex-1-en-1-yl)-6-(2,5-difluorophenyl)pyrimidin-5-amine

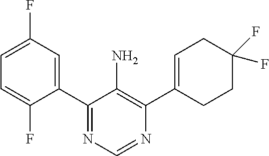 [SMILES: Nc1c(C2=CCC(F)(F)CC2)ncnc1-c1cc(F)ccc1F]

To 4-chloro-6-(2,5-difluorophenyl)pyrimidin-5-amine (2.0 g, 8.3 mmol) was added 1,4-dioxane (50 mL) and water (5.5 mL) and the mixture was sparged with nitrogen for 10 min. To the mixture was added 2-(4,4-difluorocyclohex-1-en-1-yl)-4,4,5,5-tetramethyl-1,3,2-dioxaborolane (3.0 g, 12 mmol), potassium carbonate (3.4 g, 25 mmol), and [1,1′-Bis(diphenylphosphino)ferrocene]dichloropalladium(II), complex with dichloromethane (0.68 g, 0.83 mmol), and the solution was sparged with nitrogen for 2 min. The flask was sealed under a nitrogen atmosphere and heated to 90° C. for 4 h. The reaction mixture was cooled to ambient temperature and diluted with ethyl acetate (100 mL). The organic layer was washed with saturated ammonium chloride (2×50 mL) and water (100 mL), and then dried over anhydrous magnesium sulfate, filtered, and concentrated in vacuo. Purification of the residue by column chromatography, eluting with 5-95% ethyl acetate in heptane, afforded the title compound as a light-yellow solid (2.3 g, 86% yield): MS (ES+) m/z 324.2 (M+1)

### Step 3. Preparation of 4-(4,4-difluorocyclohexyl)-6-(2,5-difluorophenyl)pyrimidin-5-amine

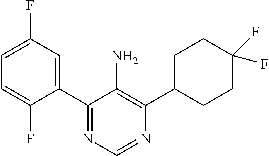 [SMILES: Nc1c(-c2cc(F)ccc2F)ncnc1C1CCC(F)(F)CC1]

To a mixture of 4-(4,4-difluorocyclohex-1-en-1-yl)-6-(2,5-difluorophenyl)pyrimidin-5-amine (1.8 g, 5.50 mmol) in methanol (55 mL) and ethyl acetate (55 mL) was degassed with nitrogen for 10 minutes. The reaction mixture was added 10% palladium on carbon (0.88 g) and placed under a hydrogen atmosphere at ambient temperature for 16 h. The reaction was stirred at 65° C. for 30 minutes. After cooling to ambient temperature, the mixture was diluted with ethyl acetate (50 mL), filtered through a bed of Celite and the filtrate was concentrated in vacuo. The resulting residue was used as is in the next step (1.34 g, 75% yield): MS (ES+) m/z 326.2 (M+1).

### Step 4. Preparation of N-(4-(4,4-difluorocyclohexyl)-6-(2,5-difluorophenyl)pyrimidin-5-yl)-5-fluoro-6-methoxynicotinamide

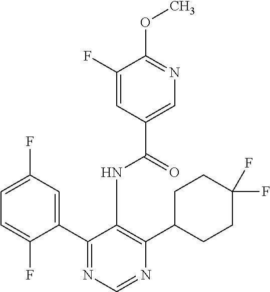 [SMILES: COc1ncc(C(=O)Nc2c(-c3cc(F)ccc3F)ncnc2C2CCC(F)(F)CC2)cc1F]

To a mixture of 4-(4,4-difluorocyclohexyl)-6-(2,5-difluorophenyl)pyrimidin-5-amine (0.092 g, 0.28 mmol), 5-fluoro-6-methoxynicotinic acid (0.097 g, 0.56 mmol) and 2-chloro-1-methylpyridinium iodide (0.22 g, 0.85 mmol) and N,N-diisopropylethylamine (0.49 mL, 2.8 mmol) was added anhydrous tetrahydrofuran (2.4 mL). The reaction mixture was stirred at 65° C. for 3 h. After cooling to ambient temperature, the mixture was diluted with methanol (5 mL) and 1M sodium hydroxide (2 mL). The mixture was stirred at ambient temperature for 30 min before it was diluted with ethyl acetate (20 mL). The organic layer was washed with saturated ammonium chloride solution (2×30 mL), dried over anhydrous magnesium sulfate, filtered, and concentrated in vacuo. Purification of the residue by preparative HPLC, eluting with a gradient of 15 to 95% acetonitrile in water with 0.5% formic acid, provided the title compound as a colorless solid (0.090 g, 67% yield): +H-NMR (400 MHz; DMSO-d₆) δ 10.39 (s, 1H), 9.22 (s, 1H), 8.43 (d, J=1.9 Hz, 1H), 7.97 (dd, J=11.0, 2.0 Hz, 1H), 7.39-7.31 (i, 3H), 4.03 (, 3H), 3.26-3.19 (m, 1H), 2.15-2.07 (m, 2H), 2.01-1.82 (n, 6H); MS (ESI+) m/z 479.2 (M+1)

In a similar manner as described in examples disclosed herein, utilizing the appropriately substituted starting materials and intermediates, the following compounds were prepared:

| Example | Structure | Amount | | |
|---|---|---|---|---|
| No. | Name | Yield | MS (ES+) m/z | ¹H NMR |
| 438 | 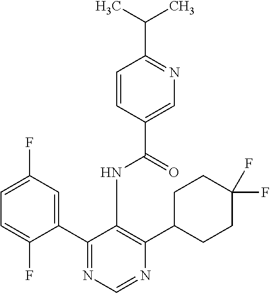 [SMILES: CC(C)c1ccc(C(=O)Nc2c(-c3cc(F)ccc3F)ncnc2C2CCC(F)(F)CC2)cn1] N-(4-(4,4- difluorocyclohexyl)-6- (2,5-difluorophenyl)pyrimidin- 5-yl)-6-isopropylnicotinamide | 0.11 g 61% | 473.2 (M + 1) | ¹H-NMR (400 MHz; DMSO-d₆) δ 10.42 (s, 1H), 9.22 (s, 1H), 8.84 (d, J = 2.1 Hz, 1H), 8.01 (dd, J = 8.1,2.3 Hz, 1H), 7.42 (d, J = 8.1 Hz, 1H), 7.38-7.31 (m, 3H), 3.27-3.20 (m, 1H), 3.09 (7, J = 6.9 Hz, 1H), 2.12-2.11 (m, 2H), 2.03-1.88 (m, 6H), 1.24 (d, J = 6.9 Hz, 6H) |
| 439 | 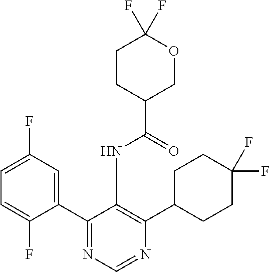 [SMILES: O=C(Nc1c(-c2cc(F)ccc2F)ncnc1C1CCC(F)(F)CC1)C1CCC(F)(F)OC1] N-(4-(4,4- difluorocyclohexyl)-6- (2,5-difluorophenyl)pyrimidin- 5-yl)-6,6-difluorotetrahydro-2H- pyran-3-carboxamide | 0.27 g 92% | 474.2 (M + 1) | ¹H-NMR (400 MHz; DMSO-d₆): δ 9.84 (s, 1H), 9.16 (s, 1H), 7.44- 7.35 (m, 2H), 7.27 (ddd, J = 8.4, 5.7, 2.7 Hz, 1H), 4.16 (dd, J = 10.6, 2.4 Hz, 1H), 3.92 (dt, J = 12.4, 6.3 Hz, 1H), 3.74 (td, J = 15.0, 12.5 Hz, 1H), 3.12-3.05 (m, 1H), 2.15-2.03 (m, 4H), 1.97-1.83 (m, 8H) |
| 440 | 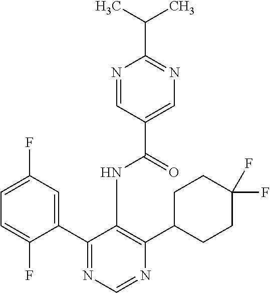 [SMILES: CC(C)c1ncc(C(=O)Nc2c(-c3cc(F)ccc3F)ncnc2C2CCC(F)(F)CC2)cn1] N-(4-(4,4- difluorocyclohexyl)-6- (2,5-difluorophenyl)pyrimidin- 5-yl)-2-isopropylpyrimidine-5- carboxamide | 0.087 g 48% | 474.2 (M + 1) | ¹H-NMR (400 MHz, DMSO-d₆) δ 10.60 (s, 1H), 9.23 (s, 1H), 9.01 (s, 2H), 7.42-7.33 (m, 3H), 3.30-3.16 (m, 2H), 2.11-1.88 (m, 8H), 1.29 (d, J = 6.9 Hz, 6H) |
| 441 | 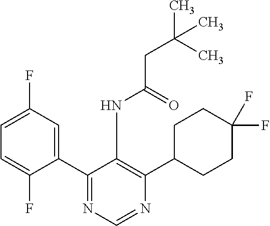 [SMILES: CC(C)(C)CC(=O)Nc1c(-c2cc(F)ccc2F)ncnc1C1CCC(F)(F)CC1] N-(4-(4,4- difluorocyclohexyl)-6- (2,5-difluorophenyl)pyrimidin- 5-yl)-3,3-dimethylbutanamide | 0.087 g 55% | 424.2 (M + 1) | ¹H-NMR (400 MHz; DMSO-d₆) δ 9.72 (s, 1H), 9.13 (s, 1H), 7.37 (t, J = 6.2 Hz, 2H), 7.28-7.24 (m, 1H), 3.14-3.11 (m, 1H), 2.18 (m, 2H), 2.08 (s, 2H), 1.85 (m, 6H), 0.84 (s, 9H) |
| 442 | 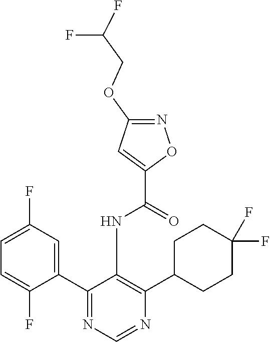 [SMILES: O=C(Nc1c(-c2cc(F)ccc2F)ncnc1C1CCC(F)(F)CC1)c1cc(OCC(F)F)no1] N-(4-(4,4- difluorocyclohexyl)- 6-(2,5-difluorophenyl) pyrimidin-5-yl)-3- (2,2-difluoroethoxy) isoxazole-5-carboxamide | 0.079 g 64% | 501.2 (M + 1) | ¹H-NMR (400 MHz; DMSO-d₆) δ 10.92 (s, 1H), 9.24 (s, 1H), 7.39- 7.32 (m, 3H), 7.05 (s, 1H), 6.42 (tt, J = 53.9, 3.1 Hz, 1H), 4.59 (td, J = 15.0, 3.1 Hz, 2H), 3.23-3.17 (m, 1H), 2.12-2.08 (m, 2H), 2.03-1.86 (m, 6H) |
| 443 | 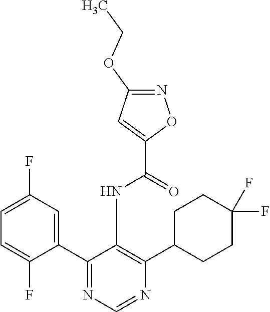 [SMILES: CCOc1cc(C(=O)Nc2c(-c3cc(F)ccc3F)ncnc2C2CCC(F)(F)CC2)on1] N-(4-(4,4- difluorocyclohexyl)- 6-(2,5-difluorophenyl) pyrimidin-5-yl)-3- ethoxyisoxazole-5-carboxamide | 0.061 g 49% | 465.2 (M + 1) | ¹H-NMR (400 MHz; DMSO-d₆) δ 10.86 (s, 1H), 9.23 (s, 1H), 7.39- 7.31 (m, 3H), 6.89 (s, 1H), 4.28 (q, J = 7.0 Hz, 2H), 3.23-3.16 (m, 1H), 2.12-2.08 (m, 2H), 2.03-1.81 (m, 6H), 1.35 (t, J = 7.0 Hz, 3H) |
| 444 | 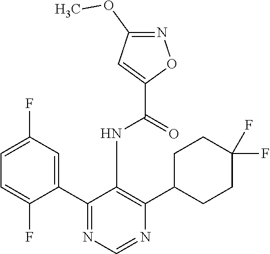 N-(4-(4,4- difluorocyclohexyl)-6- (2,5-difluorophenyl)pyrimidin- 5-yl)-3-methoxyisothiazole-5- carboxamide | 0.048 g 66% | 467.2 (M + 1) | ¹H-NMR (400 MHz; DMSO-d₆) δ 10.67 (s, 1H), 9.24 (s, 1H), 7.39- 7.32 (m, 4H), 3.96 (s, 3H), 3.22 (tt, J = 9.4, 4.7 Hz, 1H), 2.16-2.08 (m, 2H), 2.03-1.81 (m, 6H) |

### Example 445

### Synthesis of N-(4-(4,4-difluorocyclohexyl)-6-(2,5-difluorophenyl)pyrimidin-5-yl)-6-isopropoxynicotinamide

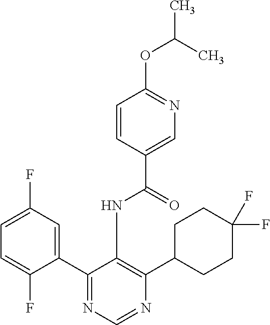 [SMILES: CC(C)Oc1ccc(C(=O)Nc2c(-c3cc(F)ccc3F)ncnc2C2CCC(F)(F)CC2)cn1]

### Step 1. Preparation of N-(4-(4,4-difluorocyclohexyl)-6-(2,5-difluorophenyl)pyrimidin-5-yl)-6-fluoronicotinamide

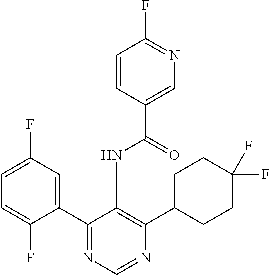 [SMILES: O=C(Nc1c(-c2cc(F)ccc2F)ncnc1C1CCC(F)(F)CC1)c1ccc(F)nc1]

To a mixture of 4-(4,4-difluorocyclohexyl)-6-(2,5-difluorophenyl)pyrimidin-5-amine (0.15 g, 0.46 mmol), 6-fluoronicotinic acid (0.098 g, 0.69 mmol) and 2-chloro-1-methylpyridinium iodide (0.35 g, 1.4 mmol) and N,N-diisopropylethylamine (0.48 mL, 2.8 mmol) was added anhydrous tetrahydrofuran (4.6 mL). The reaction mixture was stirred at 68° C. for 3 h. After cooling to ambient temperature, the mixture was diluted with methanol (5 mL) and 1M sodium hydroxide (2 mL). The mixture was stirred at ambient temperature for 30 min before it was diluted with ethyl acetate (20 mL). The organic layer was washed with water (2×30 mL), dried over anhydrous magnesium sulfate, filtered, and concentrated in vacuo. The resulting residue was used as is in the next step (0.21 g, 100% yield, crude).

### Step 2. Preparation of N-(4-(4,4-difluorocyclohexyl)-6-(2,5-difluorophenyl)pyrimidin-5-yl)-6-isopropoxynicotinamide

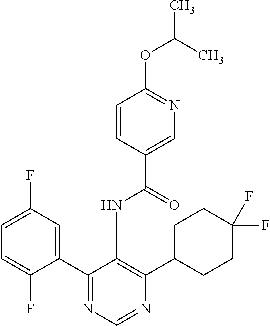 [SMILES: CC(C)Oc1ccc(C(=O)Nc2c(-c3cc(F)ccc3F)ncnc2C2CCC(F)(F)CC2)cn1]

To N-(4-(4,4-difluorocyclohexyl)-6-(2,5-difluorophenyl)pyrimidin-5-yl)-6-fluoronicotinamide (0.21 g, 0.46 mmol) was added anhydrous isopropanol (3.8 mL), N,N-dimethylformamide (3.8 mL), and 60% sodium hydride dispersion in mineral oil (0.18 g, 4.6 mmol). The solution was stirred at 45° C. for 2 h. The reaction mixture was diluted with ethyl acetate (20 mL) and washed with saturated ammonium chloride solution (2×20 mL). The organic layer was dried over anhydrous magnesium sulfate, filtered, and concentrated in vacuo. Purification of the residue by column chromatography, eluting with 5-60% ethyl acetate in heptane, afforded the title compound as a colorless solid (0.14 g, 61% yield): ¹H-NMR (400 MHz; DMSO-d₆) S 10.27 (s, 1H), 9.21 (s, 1H), 8.59 (d, J=1.4 Hz, 1H), 7.99 (dd, J=8.6, 1.9 Hz, 1H), 7.34 (t, J=5.0 Hz, 3H), 6.83 (d, J=8.7 Hz, 1H), 5.31 (dt, J=12.2, 6.1 Hz, 1H), 3.23-3.20 (m, 1H), 2.11 (m, 2H), 1.99-1.88 (m, 6H), 1.31 (d, J=6.1 Hz, 6H); MS (ES+) m/z 489.2 (M+1).

### Example 446

### Synthesis of N-(4-(4,4-difluorocyclohexyl)-6-(2,5-difluorophenyl)pyrimidin-5-yl)-5-fluoro-6-isopropoxynicotinamide

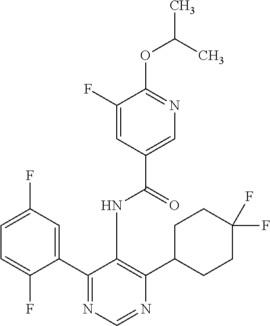 [SMILES: CC(C)Oc1ncc(C(=O)Nc2c(-c3cc(F)ccc3F)ncnc2C2CCC(F)(F)CC2)cc1F]

### Step 1. Preparation of N-(4-(4,4-difluorocyclohexyl)-6-(2,5-difluorophenyl)pyrimidin-5-yl)-5,6-difluoronicotinamide

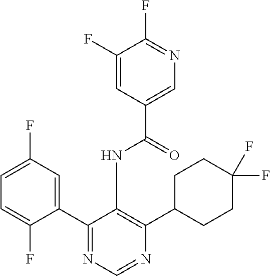 [SMILES: O=C(Nc1c(-c2cc(F)ccc2F)ncnc1C1CCC(F)(F)CC1)c1cnc(F)c(F)c1]

To a mixture of 4-(4,4-difluorocyclohexyl)-6-(2,5-difluorophenyl)pyrimidin-5-amine (0.15 g, 0.46 mmol), 2,3-difluoropyridine-5-carboxylic acid (0.11 g, 0.69 mmol) and 2-chloro-1-methylpyridinium iodide (0.35 g, 1.4 mmol) and N,N-diisopropylethylamine (0.48 mL, 2.8 mmol) was added anhydrous tetrahydrofuran (4.6 mL). The reaction mixture was stirred at 68° C. for 3 h. After cooling to ambient temperature, the mixture was diluted with methanol (5 mL) and 1M sodium hydroxide (2 mL). The mixture was stirred at ambient temperature for 30 min before it was diluted with ethyl acetate (20 mL). The organic layer was washed with saturated water (2×30 mL), dried over anhydrous magnesium sulfate, filtered and concentrated in vacuo. The resulting residue was used as is in the next step (0.22 g, 100% yield, crude)

### Step 2. Preparation of N-(4-(4,4-difluorocyclohexyl)-6-(2,5-difluorophenyl)pyrimidin-5-yl)-5-fluoro-6-isopropoxynicotinamide

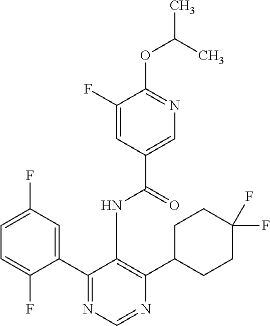 [SMILES: CC(C)Oc1ncc(C(=O)Nc2c(-c3cc(F)ccc3F)ncnc2C2CCC(F)(F)CC2)cc1F]

To a solution of N-(4-(4,4-difluorocyclohexyl)-6-(2,5-difluorophenyl)pyrimidin-5-yl)-6-fluoronicotinamide (0.21 g, 0.46 mmol) was added anhydrous isopropanol (4.6 mL), N,N-Dimethylformamide (4.6 mL), and 60% sodium hydride dispersion in mineral oil (0.18 g, 4.6 mmol). The solution was stirred at ambient temperature for 2 h. The reaction mixture was diluted with ethyl acetate (20 mL) and washed with saturated ammonium chloride solution (2×20 mL). The organic layer was dried over anhydrous magnesium sulfate, filtered, and concentrated in vacuo. Purification of the residue by column chromatography, eluting with 5-60% ethyl acetate in heptane, afforded the title compound as an off-white solid (0.050 g, 21% yield): ¹H-NMR (400 MHz; DMSO-d₆) δ 10.36 (s, 1H), 9.22 (s, 1H), 8.41 (d, J=1.9 Hz, 1H), 7.94 (dd, J=11.0, 1.9 Hz, 1H), 7.38-7.32 (m, 3H), 5.39 (hept, J=6.2 Hz, 1H), 3.26-3.19 (m, 1H), 2.12 (m, 2H), 2.03-1.88 (m, 6H), 1.35 (d, J=6.2 Hz, 6H); MS (ES+) m/z 507.2 (M+1).

### Example 447 and 448

### Synthesis of N-(4-(2,5-difluorophenyl)-6-(5,5-difluorotetrahydro-2H-pyran-2-yl)pyrimidin-5-yl)-2-isopropylpyrimidine-5-carboxamide P1/P2

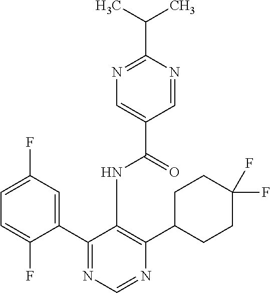 [SMILES: CC(C)c1ncc(C(=O)Nc2c(-c3cc(F)ccc3F)ncnc2C2CCC(F)(F)CC2)cn1]

### Step 1. Preparation of tert-butyl (4,6-dichloropyrimidin-5-yl)carbamate

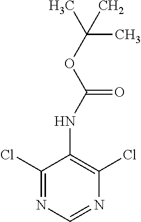 [SMILES: CC(C)(C)OC(=O)Nc1c(Cl)ncnc1Cl]

To a solution of 4,6-dichloropyrimidine-5-carboxylic acid (3.5 g, 18 mmol) in tert-butanol (91 mL) and N,N-dimethylformamide (60 mL) was added diphenylphosphonic azide (5.8 mL, 45 mmol), and triethylamine (6.3 mL, 45 mmol). The solution was heated at 85° C. for 2 h. The reaction mixture was cooled to ambient temperature and diluted with ethyl acetate (400 mL). The reaction mixture was washed with saturated sodium bicarbonate solution (3×250 mL), water (250 mL), and brine (250 mL). The organic layer was dried over anhydrous magnesium sulfate, filtered, and concentrated in vacuo. Purification of the residue by reverse-phased column chromatography, eluting with 5-95% acetonitrile in water, afforded the title compound as a yellow solid (1.5 g, 32% yield): MS (ES+) m/z 262.0 (M+1), 264.0 (M+1).

### Step 2. Preparation of tert-butyl (4-chloro-6-(2,5-difluorophenyl)pyrimidin-5-yl)carbamate

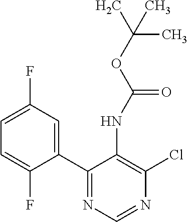 [SMILES: CC(C)(C)OC(=O)Nc1c(Cl)ncnc1-c1cc(F)ccc1F]

To tert-butyl N-(4,6-dichloropyrimidin-5-yl)carbamate (0.70 g, 2.7 mmol) was added 1,4-dioxane (12 mL) and water (1.3 mL) and the mixture was sparged with nitrogen for 10 min. To the mixture was added 2.5-difluorophenylboronc acid (0.44 g, 2.8 mmol), potassium carbonate (1.1 g, 8.0 mmol), and [1,1′-bis(diphenylphosphino)ferrocene]dichloropalladium(II), complex with dichloromethane (0.22 g, 0.27 mmol), and the solution was sparged with nitrogen for 2 min. The flask was sealed under a nitrogen atmosphere and heated to 68° C. for 3 h. The reaction mixture was cooled to ambient temperature and diluted with ethyl acetate (50 mL). The organic layer was washed with saturated ammonium chloride (2×50 mL) and water (50 mL), dried over anhydrous magnesium sulfate, filtered, and concentrated in vacuo. Purification of the residue by reverse-phased column chromatography, eluting with 5-95% acetonitrile in water, afforded the title compound as a light-yellow solid (0.35 g, 39% yield): MS (ES+) m/z 342.2 (M+1), 344.2 (M+1).

### Step 3. Preparation of tert-butyl (4-(2,5-difluorophenyl)-6-(5,5-difluorotetrahydro-2H-pyran-2-yl)pyrimidin-5-yl)carbamate

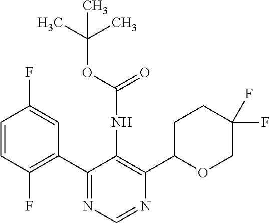 [SMILES: CC(C)(C)OC(=O)Nc1c(-c2cc(F)ccc2F)ncnc1C1CCC(F)(F)CO1]

To a vial containing tert-butyl (4-chloro-6-(2,5-difluorophenyl)pyrimidin-5-yl)carbamate (0.20 g, 0.59 mmol) was added 5,5-difluorooxane-2-carboxylic acid (0.15 g, 0.88 mmol), (4,4″-di-t-butyl-2,2″-bipyridine)bis[3,5-difluoro-2-[5-trifluoromethyl-2-pyridinyl-kN)phenyl-kC]iridium(II) hexafluorophosphate (0.0065 g, 0.0059 mmol), dichloro(dimethoxyethane)nickel (0.013 g, 0.059 mmol), 4-tert-butyl-2-(4-tert-butyl-2-pyridyl)pyridine (0.035 g, 0.088 mmol), cesium carbonate (0.38 g, 1.2 mmol), and N,N-dimethylformamide (15 mL). The vial was sealed, and the reaction mixture was stirred in front of Kessil PR160L lights (440 nm) for 20 h. The reaction mixture was diluted with ethyl acetate (250 mL) and washed with saturated ammonium chloride solution (2×50 mL), water (50 mL), and brine (50 mL). The organic layer was dried over anhydrous magnesium sulfate, filtered, and concentrated in vacuo. Purification of the residue by column chromatography, eluting with 5-70% ethyl acetate in heptane, afforded the title compound as a yellow solid (0.20 g, 81% yield): MS (ES+) m/z 428.4 (M+1).

### Step 4. Preparation of 4-(2,5-difluorophenyl)-6-(5,5-difluorotetrahydro-2H-pyran-2-yl)pyrimidin-5-amine

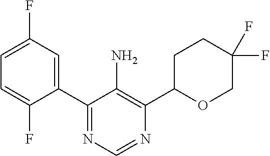 [SMILES: Nc1c(-c2cc(F)ccc2F)ncnc1C1CCC(F)(F)CO1]

To a mixture of tert-butyl (4-(2,5-difluorophenyl)-6-(5,5-difluorotetrahydro-2H-pyran-2-yl)pyrimidin-5-yl)carbamate (0.20 g, 0.47 mmol) in 1,4-dioxane (0.95 mL) was added 4 M hydrogen chloride in 1,4-dioxane (1.2 mL, 4.7 mmol). The reaction mixture was stirred for 20 h. The reaction mixture was diluted with ethyl acetate (20 mL) and washed with saturated ammonium hydroxide solution (2×20 mL), and brine (20 mL). The organic layer was dried over anhydrous magnesium sulfate, filtered, and concentrated in vacuo. Purification of the residue by column chromatography, eluting with 5-50% ethyl acetate in heptane, afforded the title compound as a light-yellow solid (0.12 g, 79% yield): MS (ES+) m/z 328.2 (M+1).

### Step 5. Preparation of N-(4-(2,5-difluorophenyl)-6-(5,5-difluorotetrahydro-2H-pyran-2-yl)pyrimidin-5-yl)-2-isopropylpyrimidine-5-carboxamide

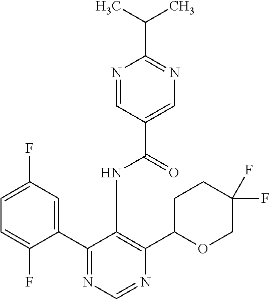 [SMILES: CC(C)c1ncc(C(=O)Nc2c(-c3cc(F)ccc3F)ncnc2C2CCC(F)(F)CO2)cn1]

To a mixture of 4-(2,5-difluorophenyl)-6-(5,5-difluorotetrahydro-2H-pyran-2-yl)pyrimidin-5-amine (0.12 g, 0.37 mmol), 2-isopropylpyrimidine-5-carboxylic acid (0.092 g, 0.55 mmol), 2-chloro-1-methylpyridinium iodide (0.28 g, 1.1 mmol) and N,N-diisopropylethylamine (0.39 mL, 2.2 mmol) added anhydrous tetrahydrofuran (3.7 mL). The reaction mixture was stirred at 68° C. for 3 h. After cooling to ambient temperature, the mixture was diluted with methanol (5 mL) and 1M sodium hydroxide (2 mL). The mixture was stirred at ambient temperature for 30 minutes and then diluted with ethyl acetate (20 mL). The organic layer was washed with saturated ammonium chloride solution (2×30 mL), dried over anhydrous magnesium sulfate, filtered, and concentrated in vacuo. Purification of the residue by column chromatography, eluting with a gradient of 5 to 80% ethyl acetate in heptane, afforded the title compound a colorless solid (0.11 g, 61% yield): ¹H-NMR (400 MHz; DMSO-d₆) δ 10.63 (s, 1H), 9.32 (s, 1H), 9.01 (s, 2H), 7.40 (q, J=7.4 Hz, 3H), 4.99 (d, J=8.5 Hz, 1H), 3.98-3.93 (m, 1H), 3.83 (dt, J=19.6, 12.1 Hz, 1H), 3.21 (7, J=6.9 Hz, 1H), 2.30-2.14 (m, 3H), 2.08-2.00 (m, 1H), 1.29 (d, J=6.9 Hz, 6H); MS (ES+) m/z 476.2 (M+1).

### Step 6. Preparation of N-(4-(2,5-difluorophenyl)-6-(5,5-difluorotetrahydro-2H-pyran-2-yl)pyrimidin-5-yl)-2-isopropylpyrimidine-5-carboxamide P1/P2

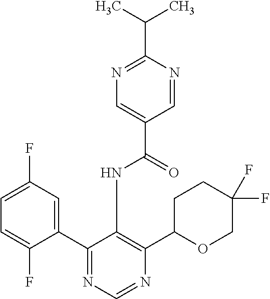 [SMILES: CC(C)c1ncc(C(=O)Nc2c(-c3cc(F)ccc3F)ncnc2C2CCC(F)(F)CO2)cn1]

N-(4-(2,5-difluorophenyl)-6-(5,5-difluorotetrahydro-2H-pyran-2-yl)pyrimidin-5-yl)-2-isopropylpyrimidine-5-carboxamide (0.10 g, 0.21 mmol) was purified by chiral SFC (column: DAICEL CHIRALPAK IC (250 mm×30 mm, 10 μm), eluting with 25% of ethanol containing 0.1% ammonium hydroxide in supercritical carbon dioxide, to afford peak 1 (retention time=0.971 min) as a colorless solid (0.025 g, 24% yield, 99% ee): ¹H NMR (400 MHz, DMSO-d₆) δ 10.61 (s, 1H), 9.31 (s, 1H), 9.00 (s, 2H), 7.43-7.33 (m, 3H), 5.01-4.96 (m, 1H), 4.00-3.90 (m, 1H), 3.82 (td, J=12.0, 19.6 Hz, 1H), 3.21 (td, J=6.8, 13.6 Hz, 1H), 2.32-2.12 (m, 3H), 2.07-1.99 (m, 1H), 1.29 (d, J=6.8 Hz, 6H); MS (ES+) m/z 476.2 (M+1).

Peak 2 (retention time=1.036 min) afforded a colorless solid (0.030 g, 29% yield, 95% ee): ¹H NMR (400 MHz, DMSO-d₆) δ 10.61 (s, 1H), 9.30 (s, 1H), 9.00 (s, 2H), 7.43-7.33 (m, 3H), 4.98 (d, J=7.6 Hz, 1H), 4.00-3.90 (m, 1H), 3.82 (td, J=12.0, 19.6 Hz, 1H), 3.21 (td, J=6.8 13.6 Hz, 1H), 2.31-2.15 (m, 3H), 2.08-1.97 (m, 1H), 1.29 (d, J=6.8 Hz, 6H); MS (ES+) m/z 476.2 (M+1).

In a similar manner as described in examples disclosed herein, utilizing the appropriately substituted starting materials and intermediates, the following compounds were prepared:

| Example | Structure | Amount | | |
|---|---|---|---|---|
| No. | Name | Yield | MS (ES+) m/z | ¹H NMR |
| 449 | 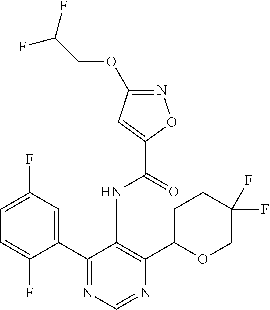 [SMILES: O=C(Nc1c(-c2cc(F)ccc2F)ncnc1C1CCC(F)(F)CO1)c1cc(OCC(F)F)no1] | 0.035 g 27% | 503.2 (M + 1) | (400 MHz, DMSO-d₆) δ 10.87 (s, 1H), 9.32 (s, 1H), 7.38 (q, J = 5.9 Hz, 3H), 7.05 (s, 1H), 6.42 (tt, J = 53.8, 3.1 Hz, 1H), 4.97-4.95 (m, 1H), 4.58 (td, J = 15.0, 3.1 Hz, 2H), 3.97-3.91 (m, 1H), 3.79 (dt, J = 21.8, 10.9 Hz, 1H), 2.34-2.12 (m, 3H), 2.08-1.99 (m, 1H) |
| | 3-(2,2-difluoroethoxy)-N- | | | |
| | (4-(2,5-difluorophenyl)-6-(5,5- | | | |
| | difluorotetrahydro-2H- | | | |
| | pyran-2-yl)pyrimidin-5- | | | |
| | yl)isoxazole-5-carboxamide | | | |
| 450 | 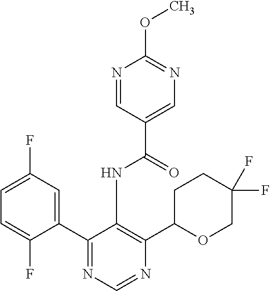 [SMILES: COc1ncc(C(=O)Nc2c(-c3cc(F)ccc3F)ncnc2C2CCC(F)(F)CO2)cn1] | 0.065 g 23% | 464.2 (M + 1) | (400 MHz, DMSO-d₆) δ 10.51 (s, 1H), 9.30 (s, 1H), 8.93 (s, 2H), 7.37 (m, 3H), 4.99-4.96 (m, 1H), 3.99 (s, 3H), 3.97- 3.92 (m, 1H), 3.87-3.76 (m, 1H), 2.30-2.01 (m, 4H) |
| | N-(4-(2,5- | | | |
| | difluorophenyl)-6-(5,5- | | | |
| | difluorotetrahydro-2H- | | | |
| | pyran-2-yl)pyrimidin-5- | | | |
| | yl)-2-methoxypyrimidine- | | | |
| | 5-carboxamide | | | |
| 451 | 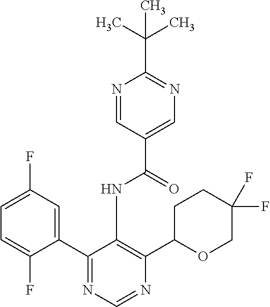 [SMILES: CC(C)(C)c1ncc(C(=O)Nc2c(-c3cc(F)ccc3F)ncnc2C2CCC(F)(F)CO2)cn1] | 0.076 g 30% | 490.2 (M + 1) | (400 MHz, DMSO-d₆) δ 10.63 (s, 1H), 9.32 (s, 1H), 9.02 (s, 2H), 7.44- 7.35 (m, 3H), 5.01-4.98 (m, 1H), 3.99-3.93 (m, 1H), 3.89-3.78 (m, 1H), 2.30-2.16 (m, 3H), 2.05-2.01 (m, 1H), 1.38 (s, 9H) |
| | 2-(tert-butyl)-N-(4-(2,5- | | | |
| | difluorophenyl)-6-(5,5- | | | |
| | difluorotetrahydro-2H- | | | |
| | pyran-2-yl)pyrimidin-5- | | | |
| | yl)pyrimidine-5-carboxamide | | | |
| 452 | 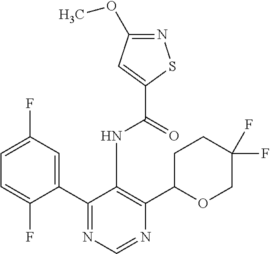 [SMILES: COc1cc(C(=O)Nc2c(-c3cc(F)ccc3F)ncnc2C2CCC(F)(F)CO2)sn1] | 0.062 g 48% | 469.2 (M + 1) | (400 MHz, DMSO-d₆) δ 10.68 (s, 1H), 9.32 (s, 1H), 7.40-7.35 (m, 4H), 4.98-4.95 (m, 1H), 3.96 (s, 3H), 3.94-3.92 (m, 1H), 3.86-3.75 (m, 1H), 2.33-2.13 (m, 3H), 2.06-1.99 (m, 1H) |
| | N-(4-(2,5- | | | |
| | difluorophenyl)-6-(5,5- | | | |
| | difluorotetrahydro-2H- | | | |
| | pyran-2-yl)pyrimidin-5-yl)-3- | | | |
| | methoxyisothiazole-5-carboxamide | | | |
| 453 | 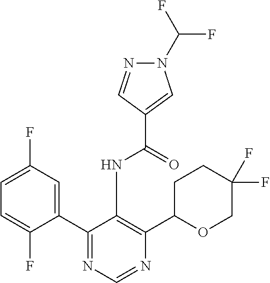 [SMILES: O=C(Nc1c(-c2cc(F)ccc2F)ncnc1C1CCC(F)(F)CO1)c1cnn(C(F)F)c1] | 0.037 g 31% | 472.2 (M + 1) | (400 MHz, MeOD) δ 9.23 (s, 1H), 8.57 (s, 1H), 8.09 (s, 1H), 7.58 (t, J = 59.4 Hz, 1H), 7.34-7.30 (m, 1H), 7.26-7.19 (m, 2H), 4.93 (m, 1H), 4.07-4.00 (m, 1H), 3.76 (ddd, J = 23.0, 12.2, 7.5 Hz, 1H), 2.38-2.30 (m, 1H), 2.27-2.10 (m, 3H) |
| | 1-(difluoromethyl)-N-(4- | | | |
| | (2,5-difluorophenyl)-6- | | | |
| | (5,5-difluorotetrahydro- | | | |
| | 2H-pyran-2-yl)pyrimidin- | | | |
| | 5-yl)-1H-pyrazole-4-carboxamide | | | |
| 454 | 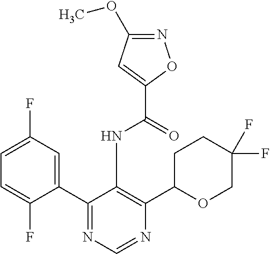 [SMILES: COc1cc(C(=O)Nc2c(-c3cc(F)ccc3F)ncnc2C2CCC(F)(F)CO2)on1] | 0.13 g 62% | 453.2 (M + 1) | (400 MHz, DMSO-d₆) δ 10.82 (s, 1H), 9.31 (s, 1H), 7.42-7.34 (m, 3H), 6.92 (s, 1H), 4.96 (dd, J = 7.1, 3.0 Hz, 1H), 3.95 (s, 3H), 3.93- 3.90 (m, 1H), 3.85-3.74 (m, 1H), 2.27-2.11 (m, 3H), 2.09-2.01 (m, 1H) |
| | N-(4-(2,5- | | | |
| | difluorophenyl)-6-(5,5- | | | |
| | difluorotetrahydro-2H- | | | |
| | pyran-2-yl)pyrimidin-5- | | | |
| | yl)-3-methoxyisoxazole- | | | |
| | 5-carboxamide | | | |
| 455 | 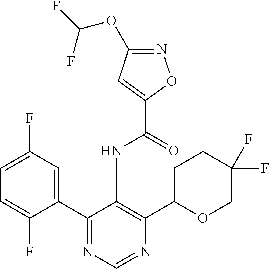 [SMILES: O=C(Nc1c(-c2cc(F)ccc2F)ncnc1C1CCC(F)(F)CO1)c1cc(OC(F)F)no1] | 0.080 g 33% | 489.2 (M + 1) | (400 MHz, DMSO-d₆) δ 11.02 (s, 1H), 9.32 (s, 1H), 7.72-7.25 (m, 5H), 4.97 (d, J = 8.8 Hz, 1H), 3.97-3.90 (m, 1H), 3.79 (dt, J = 21.7, 10.9 Hz, 1H), 2.35-2.14 (m, 3H), 2.09-2.01 (m, 1H) |
| | 3-(difluoromethoxy)-N- | | | |
| | (4-(2,5-difluorophenyl)-6-(5,5- | | | |
| | difluorotetrahydro-2H- | | | |
| | pyran-2-yl)pyrimidin-5- | | | |
| | yl)isoxazole-5-carboxamide | | | |
| 456 | 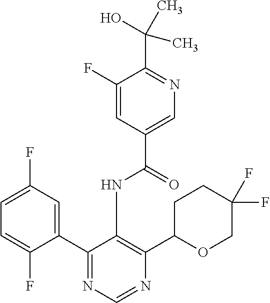 [SMILES: CC(C)(O)c1ncc(C(=O)Nc2c(-c3cc(F)ccc3F)ncnc2C2CCC(F)(F)CO2)cc1F] | 0.11 g 53% | 509.2 (M + 1) | (400 MHz, DMSO-d₆) δ 10.58 (s, 1H), 9.31 (s, 1H), 8.68 (s, 1H), 7.94 (dd, J = 11.9, 1.6 Hz, 1H), 7.38 (dt, J = 6.3, 3.0 Hz, 3H), 5.37 (s, 1H), 4.99-4.96 (m, 1H), 3.98-3.92 (m, 1H), 3.87-3.76 (m, 1H), 2.31-2.13 (m, 3H), 2.09-2.02 (m, 1H), 1.52 (s, 6H) |
| | N-(4-(2,5- | | | |
| | difluorophenyl)-6-(5,5- | | | |
| | difluorotetrahydro-2H- | | | |
| | pyran-2-yl)pyrimidin-5- | | | |
| | yl)-5-fluoro-6-(2- | | | |
| | hydroxypropan-2- | | | |
| | yl)nicotinamide | | | |
| 457 | 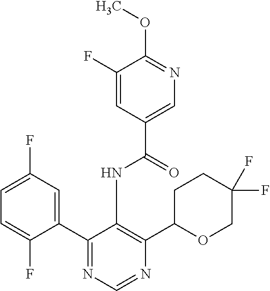 [SMILES: COc1ncc(C(=O)Nc2c(-c3cc(F)ccc3F)ncnc2C2CCC(F)(F)CO2)cc1F] | 0.019 g 11% | 481.2 (M + 1) | (400 MHz, DMSO-d₆) δ 10.43 (d, J = 0.3 Hz, 1H), 9.29 (s, 1H), 8.43 (d, J = 1.7 Hz, 1H), 7.98 (dd, J = 11.0, 1.5 Hz, 1H), 7.39-7.35 (m, 3H), 4.97-4.95 (m, 1H), 4.01 (s, 3H), 3.98-3.92 (m, 1H), 3.84-3.73 (m, 1H), 2.33-2.11 (m, 3H), 2.07-1.99 (m, 1H) |
| | N-(4-(2,5- | | | |
| | difluorophenyl)-6-(5,5- | | | |
| | difluorotetrahydro-2H- | | | |
| | pyran-2-yl)pyrimidin-5- | | | |
| | yl)-5-fluoro-6- | | | |
| | methoxynicotinamide | | | |
| 458 | 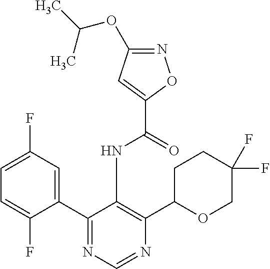 [SMILES: CC(C)Oc1cc(C(=O)Nc2c(-c3cc(F)ccc3F)ncnc2C2CCC(F)(F)CO2)on1] | 0.087 g 55% | 481.2 (M + 1) | (400 MHz, DMSO-d₆) δ 10.79 (s, 1H), 9.31 (s, 1H), 7.40-7.35 (m, 3H), 6.88 (s, 1H), 4.97-4.95 (m, 1H), 4.83 (7, J = 6.1 Hz, 1H), 3.97-3.91 (m, 1H), 3.85-3.74 (m, 1H), 2.34-2.11 (m, 3H), 2.08-1.99 (m, 1H), 1.34 (d, J = 6.1 Hz, 6H) |
| | N-(4-(2,5- | | | |
| | difluorophenyl)-6-(5,5- | | | |
| | difluorotetrahydro-2H- | | | |
| | pyran-2-yl)pyrimidin-5-yl)-3- | | | |
| | isopropoxyisoxazole-5- | | | |
| | carboxamide | | | |
| 459 | 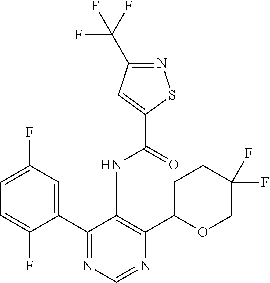 [SMILES: O=C(Nc1c(-c2cc(F)ccc2F)ncnc1C1CCC(F)(F)CO1)c1cc(C(F)(F)F)ns1] | 0.11 g 59% | 507.2 (M + 1) | (400 MHz, DMSO-d₆) δ 10.95 (s, 1H), 9.34 (s, 1H), 8.40 (s, 1H), 7.42- 7.36 (m, 3H), 5.01-4.98 (m, 1H), 3.97-3.91 (m, 1H), 3.87-3.76 (m, 1H), 2.34-2.14 (m, 3H), 2.09-1.99 (m, 1H) |
| | N-(4-(2,5- | | | |
| | difluorophenyl)-6-(5,5- | | | |
| | difluorotetrahydro-2H- | | | |
| | pyran-2-yl)pyrimidin-5-yl)-3- | | | |
| | (trifluoromethyl)isothiazole- | | | |
| | 5-carboxamide | | | |
| 460 | 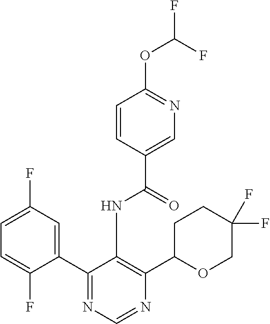 [SMILES: O=C(Nc1c(-c2cc(F)ccc2F)ncnc1C1CCC(F)(F)CO1)c1ccc(OC(F)F)nc1] | 0.12 g 68% | 499.2 (M + 1) | (400 MHz, DMSO-d₆) δ 10.49 (s, 1H), 9.31 (s, 1H), 8.65 (d, J = 2.3 Hz, 1H), 8.22 (dd, J = 8.6, 2.4 Hz, 1H), 7.79 (t, J = 72.3 Hz, 1H), 7.40-7.35 (m, 3H), 7.23 (d, J = 8.6 Hz, 1H), 4.99-4.96 (m, 1H), 3.98-3.92 (m, 1H), 3.85-3.75 (m, 1H), 2.35-2.11 (m, 3H), 2.05-1.99 (m, 1H) |
| | 6-(difluoromethoxy)-N- | | | |
| | (4-(2,5-difluorophenyl)-6-(5,5- | | | |
| | difluorotetrahydro-2H- | | | |
| | pyran-2-yl)pyrimidin-5- | | | |
| | yl)nicotinamide | | | |
| 461 | 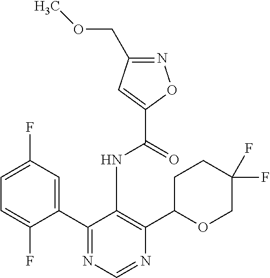 [SMILES: COCc1cc(C(=O)Nc2c(-c3cc(F)ccc3F)ncnc2C2CCC(F)(F)CO2)on1] | 0.030 g 23% | 467.2 (M + 1) | (400 MHz, DMSO-d₆) δ 10.87 (s, 1H), 9.31 (s, 1H), 7.39-7.36 (m, 3H), 7.15 (s, 1H), 4.97 (dd, J = 6.8, 3.2 Hz, 1H), 4.54 (s, 2H), 3.96-3.89 (m, 1H), 3.84-3.73 (m, 1H), 3.33 (s, 3H), 2.34- 2.11 (m, 3H), 2.09-2.00 (m, 1H) |
| | N-(4-(2,5- | | | |
| | difluorophenyl)-6-(5,5- | | | |
| | difluorotetrahydro-2H- | | | |
| | pyran-2-yl)pyrimidin-5-yl)-3- | | | |
| | (methoxymethyl)isoxazole- | | | |
| | 5-carboxamide | | | |
| 462 | 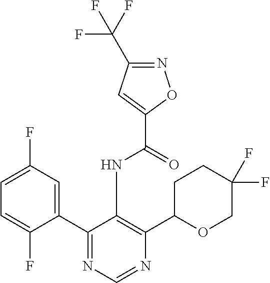 [SMILES: O=C(Nc1c(-c2cc(F)ccc2F)ncnc1C1CCC(F)(F)CO1)c1cc(C(F)(F)F)no1] | 0.062 g 38% | 491.2 (M + 1) | (400 MHz, DMSO-d₆) δ 11.19 (s, 1H), 9.33 (s, 1H), 7.84 (s, 1H), 7.41- 7.38 (m, 3H), 5.01-4.98 (m, 1H), 3.97-3.90 (m, 1H), 3.85-3.74 (m, 1H), 2.34-2.13 (m, 3H), 2.08-1.99 (m, 1H) |
| | N-(4-(2,5- | | | |
| | difluorophenyl)-6-(5,5- | | | |
| | difluorotetrahydro-2H- | | | |
| | pyran-2-yl)pyrimidin-5-yl)-3- | | | |
| | (trifluoromethyl)isoxazole- | | | |
| | 5-carboxamide | | | |
| 463 | 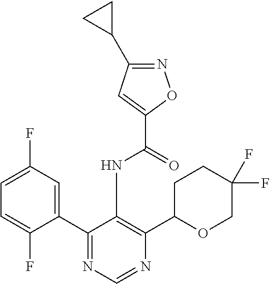 [SMILES: O=C(Nc1c(-c2cc(F)ccc2F)ncnc1C1CCC(F)(F)CO1)c1cc(C2CC2)no1] | 0.049 g 37% | 463.2 (M + 1) | (400 MHz, DMSO-d₆) δ 10.80 (s, 1H), 9.30 (s, 1H), 7.37 (ddd, J = 7.8, 5.6, 2.3 Hz, 3H), 6.92 (d, J = 7.9 Hz, 1H), 4.96-4.94 (m, 1H), 3.96-3.89 (m, 1H), 3.84-3.73 (m, 1H), 2.34-2.13 (m, 3H), 2.11-2.01 (m, 2H), 1.10-1.01 (m, 2H), 0.88-0.76 (m, 2H) |
| | 3-cyclopropyl-N-(4-(2,5- | | | |
| | difluorophenyl)-6-(5,5- | | | |
| | difluorotetrahydro-2H- | | | |
| | pyran-2-yl)pyrimidin-5- | | | |
| | yl)isoxazole-5- | | | |
| | carboxamide | | | |
| 464 | 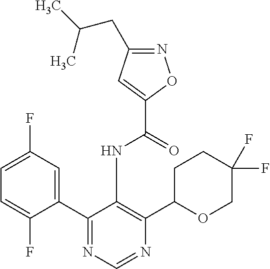 [SMILES: CC(C)Cc1cc(C(=O)Nc2c(-c3cc(F)ccc3F)ncnc2C2CCC(F)(F)CO2)on1] | 0.085 g 53% | 479.2 (M + 1) | (400 MHz, DMSO-d₆) δ 9.95 (s, 1H), 8.46 (s, 1H), 6.52 (dd, J = 7.5, 5.4 Hz, 3H), 6.20 (s, 1H), 4.13-4.11 (m, 1H), 3.10-3.04 (m, 1H), 2.99-2.88 (m, 1H), 1.71 (d, J = 7.1 Hz, 2H), 1.46-1.26 (m, 3H), 1.24-1.16 (m, 1H), 1.10 (dq, J = 13.5, 6.8 Hz, 1H), 0.06 (d, J = 6.6 Hz, 6H) |
| | N-(4-(2,5- | | | |
| | difluorophenyl)-6-(5,5- | | | |
| | difluorotetrahydro-2H- | | | |
| | pyran-2-yl)pyrimidin-5- | | | |
| | yl)-3-isobutylisoxazole- | | | |
| | 5-carboxamide | | | |
| 465 | 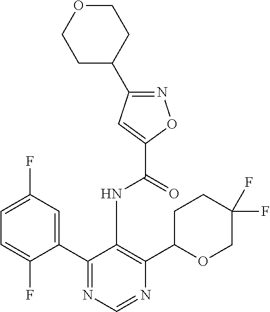 [SMILES: O=C(Nc1c(-c2cc(F)ccc2F)ncnc1C1CCC(F)(F)CO1)c1cc(C2CCOCC2)no1] | 0.058 g 34% | 507.2 (M + 1) | (400 MHz, DMSO-d₆) δ 10.84 (s, 1H), 9.31 (s, 1H), 7.38 (t, J = 6.7 Hz, 3H), 7.17 (s, 1H), 4.99- 4.96 (m, 1H), 3.96-3.89 (m, 3H), 3.85-3.74 (m, 1H), 3.44 (td, J = 11.6, 2.0 Hz, 2H), 3.10-3.02 (m, 1H), 2.34-2.11 (m, 3H), 2.07-1.99 (m, 1H), 1.86-1.82 (m, 2H), 1.72-1.62 (m, 2H) |
| | N-(4-(2,5- | | | |
| | difluorophenyl)-6-(5,5- | | | |
| | difluorotetrahydro-2H- | | | |
| | pyran-2-yl)pyrimidin-5- | | | |
| | yl)-3-(tetrahydro-2H- | | | |
| | pyran-4-yl)isoxazole-5- | | | |
| | carboxamide | | | |
| 466 | 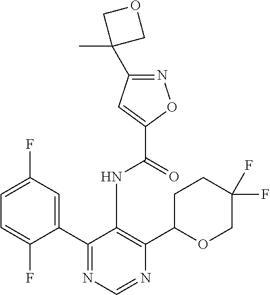 [SMILES: CC1(c2cc(C(=O)Nc3c(-c4cc(F)ccc4F)ncnc3C3CCC(F)(F)CO3)on2)COC1] | 0.032 g 20% | 493.2 (M + 1) | (400 MHz, DMSO-d₆) δ 10.90 (s, 1H), 9.32 (s, 1H), 7.39 (t, J = 6.6 Hz, 3H), 7.33 (s, 1H), 4.99- 4.97 (m, 1H), 4.79 (d, J = 6.0 Hz, 2H), 4.55 (d, J = 6.0 Hz, 2H), 3.96-3.90 (m, 1H), 3.85-3.74 (m, 1H), 2.35-2.12 (m, 3H), 2.09-2.02 (m, 1H), 1.67 (s, 3H) |
| | N-(4-(2,5- | | | |
| | difluorophenyl)-6-(5,5- | | | |
| | difluorotetrahydro-2H- | | | |
| | pyran-2-yl)pyrimidin-5- | | | |
| | yl)-3-(3-methyloxetan-3- | | | |
| | yl)isoxazole-5- | | | |
| | carboxamide | | | |
| 467 | 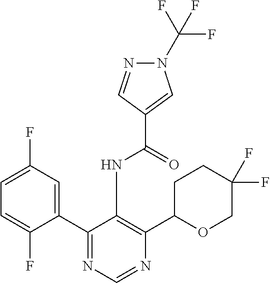 [SMILES: O=C(Nc1c(-c2cc(F)ccc2F)ncnc1C1CCC(F)(F)CO1)c1cnn(C(F)(F)F)c1] | 0.11 g 62% | 490.2 (M + 1) | (400 MHz, DMSO-d₆) δ 10.26 (s, 1H), 9.30 (s, 1H), 9.04 (s, 1H), 8.35 (s, 1H), 7.36 (t, J = 6.6 Hz, 3H), 4.94 (dd, J = 9.8, 2.3 Hz, 1H), 4.00- 3.93 (m, 1H), 3.86-3.75 (m, 1H), 2.32-2.14 (m, 3H), 2.06-1.98 (m, 1H) |
| | N-(4-(2,5- | | | |
| | difluorophenyl)-6-(5,5- | | | |
| | difluorotetrahydro-2H- | | | |
| | pyran-2-yl)pyrimidin-5- | | | |
| | yl)-1-(trifluoromethyl)- | | | |
| | 1H-pyrazole-4- | | | |
| | carboxamide | | | |

In a similar manner as described in examples disclosed herein, utilizing appropriate chiral separation condition, following compounds were prepared:

| Example | Structure | SFC | Retention time (Rₜ), ee; ¹H |
|---|---|---|---|
| No. | P1 or P2 | Condition | NMR, MS (ES+), Rₜ |
| 468 | 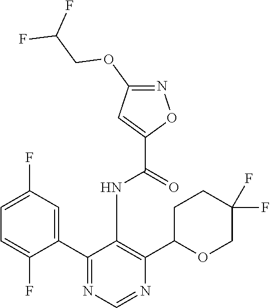 [SMILES: O=C(Nc1c(-c2cc(F)ccc2F)ncnc1C1CCC(F)(F)CO1)c1cc(OCC(F)F)no1] | column: DAICEL CHIRALPAK IC (250 mm × 30 mm, 5 μm) eluting with 15% of methanol containing 0.1% ammonium hydroxide in supercritical carbon dioxide | 0.685 min, 99%; (400 MHz, DMSO-d₆) δ 10.85 (s, 1H), 9.31 (s, 1H), 7.49-7.26 (m, 3H), 7.04 (s, 1H), 6.42 (tt, J = 3.2, 54.0 Hz, 1H), 5.04- 4.85 (m, 1H), 4.58 (dt, J = 2.8, 15.2 Hz, 2H), 4.02-3.88 (m, 1H), 3.85-3.71 (m, 1H), 2.31-2.11 (m, 3H), 2.08- 1.94 (m, 1H); MS (ES+) m/z 503.1 (M + 1) |
| | 3-(2,2-difluoroethoxy)-N- | | |
| | (4-(2,5-difluorophenyl)-6- | | |
| | (5,5-difluorotetrahydro-2H- | | |
| | pyran-2-yl)pyrimidin-5- | | |
| | yl)isoxazole-5- | | |
| | carboxamide | | |
| | P1 | | |
| 469 | 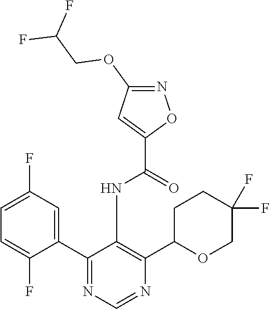 | column: DAICEL CHIRALPAK IC (250 mm × 30 mm, 5 μm) eluting with 15% of methanol containing 0.1% ammonium hydroxide in supercritical carbon dioxide | 0.857 min, 98%; (400 MHz, DMSO-d₆) δ 10.84 (s, 1H), 9.31 (s, 1H), 7.42-7.31 (m, 3H), 7.04 (s, 1H), 6.58-6.25 (m, 1H), 4.95 (d, J = 8.8 Hz, 1H), 4.58 (dt, J = 2.8, 14.8 Hz, 2H), 3.99-3.88 (m, 1H), 3.85-3.72 (m, 1H), 2.31- 2.10 (m, 3H), 2.09-1.97 (m, 1H); MS (ES+) m/z 503.1 (M + 1), |
| | 3-(2,2-difluoroethoxy)-N- | | |
| | (4-(2,5-difluorophenyl)-6- | | |
| | (5,5-difluorotetrahydro-2H- | | |
| | pyran-2-yl)pyrimidin-5- | | |
| | yl)isoxazole-5- | | |
| | carboxamide | | |
| | P2 | | |
| 470 | 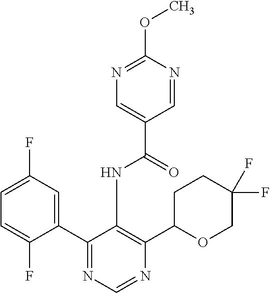 [SMILES: COc1ncc(C(=O)Nc2c(-c3cc(F)ccc3F)ncnc2C2CCC(F)(F)CO2)cn1] | column: DAICEL CHIRALPAK IC (250 mm × 30 mm, 5 μm) eluting with 15% of methanol containing 0.1% ammonium hydroxide in supercritical carbon dioxide | 1.445 min, 99%; (400 MHz, CDCl₃) δ 9.15 (s, 1H), 8.88 (s, 3H), 7.43 (ddd, J = 3.2, 5.2, 8.4 Hz, 1H), 7.13-6.98 (m, 2H), 4.82 (dd, J = 3.2, 10.4 Hz, 1H), 4.20-4.12 (m, 1H), 4.10 (s, 3H), 3.80-3.67 (m, 1H), 2.42-2.31 (m, 1H), 2.27-1.99 (m, 3H); MS (ES+) m/z 464.2 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-2-methoxypyrimidine-5- | | |
| | carboxamide | | |
| | P1 | | |
| 471 | 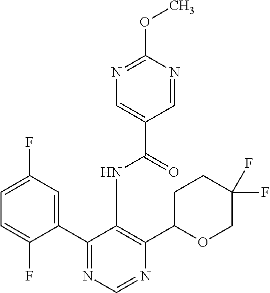 [SMILES: COc1ncc(C(=O)Nc2c(-c3cc(F)ccc3F)ncnc2C2CCC(F)(F)CO2)cn1] | column: DAICEL CHIRALPAK IC (250 mm × 30 mm, 5 μm) eluting with 15% of methanol containing 0.1% ammonium hydroxide in supercritical carbon dioxide | 1.590 min, 99%; (400 MHz, CDCl₃) δ 9.15 (s, 1H), 8.88 (s, 3H), 7.43 (ddd, J = 3.2, 5.2, 8.4 Hz, 1H), 7.15-6.92 (m, 2H), 4.82 (dd, J = 2.8, 10.4 Hz, 1H), 4.20-4.11 (m, 1H), 410 (s, 3H), 3.74 (td, J = 12.4, 17.2 HZ, 1H), 2.43- 2.30(m, 1H), 2.28-1.98 (m, 3H); MS (ES+) m/z 464.2 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-2-methoxypyrimidine-5- | | |
| | carboxamide | | |
| | P2 | | |
| 472 | 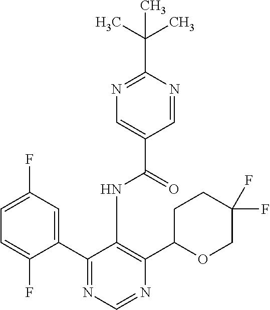 [SMILES: CC(C)(C)c1ncc(C(=O)Nc2c(-c3cc(F)ccc3F)ncnc2C2CCC(F)(F)CO2)cn1] | column: DAICEL CHIRALPAK IC (250 mm × 30 mm, 5 μm) eluting with 20% of isopropanol containing 0.1% ammonium hydroxide in supercritical carbon dioxide | 1.008 min, 98%; (400 MHz, DMSO-d₆) δ 10.62 (s, 1H), 9.31 (s, 1H), 9.01 (s, 2H), 7.38 (dd, J = 5.2, 7.6 Hz, 3H), 4.98 (d, J = 8.4 Hz, 1H), 4.00-3.90 (m, 1H), 3.83 (td, J = 12.4, 19.2 Hz, 1H), 2.30- 2.13 (m, 3H), 2.03 (d, J = 10.0 Hz, 1H), 1.37 (s, 9H); MS (ES+) m/z 490.3 (M + 1) |
| | 2-(tert-butyl)-N-(4-(2,5- | | |
| | difluorophenyl)-6-(5,5- | | |
| | difluorotetralydro-2H- | | |
| | pyran-2-yl)pyrimidin-5- | | |
| | yl)pyrimicline-5- | | |
| | carboxamide | | |
| | P1 | | |
| 473 | 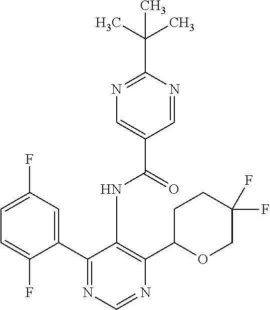 [SMILES: CC(C)(C)c1ncc(C(=O)Nc2c(-c3cc(F)ccc3F)ncnc2C2CCC(F)(F)CO2)cn1] | column: DAICEL CHIRALPAK IC (250 mm × 30 mm, 5 μm) eluting with 20% of isopropanol containing 0.1% ammonium hydroxide in supercritical carbon dioxide | 1.187 min, 99%; (400 MHz, DMSO-d₆) δ 10.62 (s, 1H), 9.31 (s, 1H), 9.01 (s, 2H), 7.44-7.32 (m, 3H), 4.98 (d, J = 9.2 Hz, 1H), 4.00-3.90 (m, 1H), 3.90-3.76 (m, 1H), 2.30-2.14 (m, 3H), 2.03 (d, J = 10.4 Hz, 1H), 1.37 (s, 9H); MS (ES+) m/z 490.3 (M + 1) |
| | 2-(tert-butyl)-N-(4-(2,5- | | |
| | difluorophenyl)-6-(5,5- | | |
| | difluorotetralydro-2H- | | |
| | pyran-2-yl)pyrimidin-5- | | |
| | yl)pyrimicline-5- | | |
| | carbooxamide | | |
| | P2 | | |
| 474 | 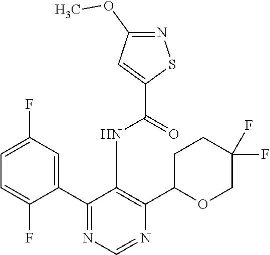 | column: DAICEL CHIRALPAK IC (250 mm × 30 mm, 5 μm) eluting with 20% of methanol containing 0.1% ammonium hydroxide in supercritical carbon dioxide | 0.869 min, 99%; (400 MHz, CDCl₃) δ 9.15 (s, 1H), 8.76 (s, 1H), 7.41 (ddd, J = 3.2, 5.2, 8.0 Hz, 1H), 7.16-7.00 (m, 2H), 6.89 (s, 1H), 4.81 (dd, J = 3.6, 10.4 Hz, 1H), 4.16 (dtd, J = 2.8, 6.0, 12.4 Hz, 1H), 4.04 (s, 3H), 3.83- 3.66 (m, 1H), 2.48-2.35 (m, 1H), 2.31-2.01 (m, 3H); MS (ES+) m/z 469.2 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-3-methoxyisothiazole- | | |
| | 5-carboxamide | | |
| | P1 | | |
| 475 | 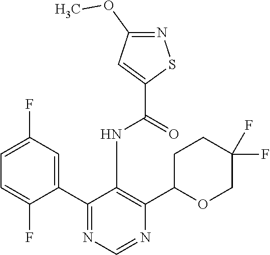 | column: DAICEL CHIRALPAK IC (250 mm × 30 mm, 5 μm) eluting with 20% of methanol containing 0.1% ammonium hydroxide in supercritical carbon dioxide | 0.993 min, 99%; (400 MHz, CDCl₃) δ 9.07 (s, 1H), 8.68 (s, 1H), 7.33 (ddd, J = 3.2, 5.2, 8.4 Hz, 1H), 6.99 (dt, J = 4.4, 9.2 Hz, 2H), 6.81 (s, 1H), 4.73 (dd, J = 3.6, 10.0 Hz, 1H), 3.96 (s, 3H), 3.76- 3.57 (m, 1H), 2.32 (td, J = 3.6, 12.8 Hz, 1H), 2.22-1.90 (m, 3H); MS (ES+) m/z 469.2 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-3-methoxyisothiazole- | | |
| | 5-carboxamide | | |
| | P2 | | |
| 476 | 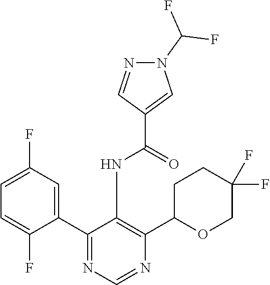 [SMILES: O=C(Nc1c(-c2cc(F)ccc2F)ncnc1C1CCC(F)(F)CO1)c1cnn(C(F)F)c1] | column: DAICEL CHIRALPAK AD (250 mm × 30 mm, 10 μm) eluting with 20% of isopropyl alcohol containing 0.1% ammonium hydroxide in supercritical carbon dioxide | 1.054 min, 99%; (400 MHz, CDCl₃) δ 9.14 (s, 1H), 8.63 (s, 1H), 8.23 (s, 1H), 7.93 (s, 1H), 7.42 (ddd, J = 3.2, 5.2, 8.0 Hz, 1H), 7.35-6.98 (m, 3H), 4.81 (dd, J = 5.6, 8.0 Hz, 1H), 4.18 (dtd, J = 2.8, 6.0, 12.4 Hz, 1H), 3.85-3.66 (m, 1H), 2.38 (qd, J = 3.6, 16.8 Hz, 1H), 2.26-1.99 (m, 3H); MS (ES+) m/z 472.1 (M + 1) |
| | 1-(difluoromethyl)-N-(4- | | |
| | (2,5-difluorophenyl)-6-(5,5- | | |
| | difluorotetrahydro-2H- | | |
| | pyran-2-yl)pyrimidin-5-yl)- | | |
| | 1H-pyrazole-4- | | |
| | carboxamide | | |
| | P1 | | |
| 477 | 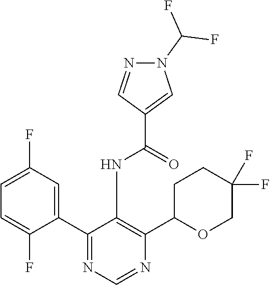 [SMILES: O=C(Nc1c(-c2cc(F)ccc2F)ncnc1C1CCC(F)(F)CO1)c1cnn(C(F)F)c1] | column: DAICEL CHIRALPAK AD (250 mm × 30 mm, 10 μm) eluting with 20% of methanol containing 0.1% ammonium hydroxide in supercritical carbon dioxide | 1.243 min, 99%; (400 MHz, CDCl₃) δ 9.14 (s, 1H), 8.63 (s, 1H), 8.23 (s, 1H), 7.93 (s, 1H), 7.42 (ddd, J = 3.2, 5.2, 8.0 Hz, 1H), 7.35-6.98 (m, 3H), 4.81 (dd, J = 5.6, 8.0 Hz, 1H), 4.18 (dtd, J = 2.8, 6.0, 12.4 Hz, 1H), 3.85-3.66 (m, 1H), 2.38 (qd, J = 3.6, 16.8 Hz, 1H), 2.26-1.99 (m, 3H); MS (ES+) m/z 472.1 (M + 1) |
| | 1-(difluoromethyl)-N-(4- | | |
| | (2,5-difluorophenyl)-6-(5,5- | | |
| | difluorotetrahydro-2H- | | |
| | pyran-2-yl)pyrimidin-5-yl)- | | |
| | 1H-pyrazole-4- | | |
| | carboxamide | | |
| | P2 | | |
| 478 | 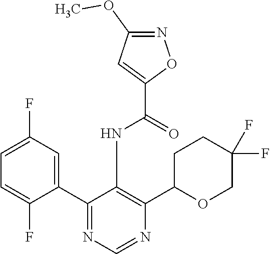 | column: DAICEL CHIRALPAK IC (250 mm × 30 mm, 10 μm) eluting with 20% of methanol containing 0.1% ammonium hydroxide in supercritical carbon dioxide | 0.871 min, 99%; (400 MHz, DMSO-d₆) δ 10.81 (s, 1H), 9.31 (s, 1H), 7.40-7.35 (m, 3H), 6.91 (s, 1H), 4.97-4.94 (m, 1H), 3.95 (s, 3H), 3.84- 3.73 (m, 1H), 2.33-2.15 (m, 3H), 2.05-2.02 (m, 1H); MS (ES+) m/z 453.1 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-3-methoxyisoxazole-5- | | |
| | carboxamide | | |
| | P1 | | |
| 479 | 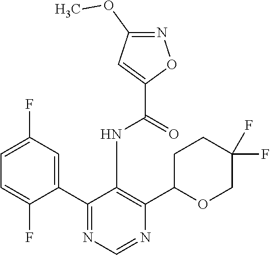 | column: DAICEL CHIRALPAK IC (250 mm × 30 mm, 10 μm) eluting with 20% of methanol containing 0.1% ammonium hydroxide in supercritical carbon dioxide | 1.111 min, 99%; (400 MHz, CDCl₃) δ 10.81 (s, 1H), 9.31 (s, 1H), 7.40-7.35 (m, 3H), 6.91 (s, 1H), 4.97-4.94 (m, 1H), 3.84-3.73 (m, 1H), 2.34- 2.11 (m, 3H), 2.07-2.00 (m, 1H); MS (ES+) m/z 453.1 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-3-methoxyisoxazole-5- | | |
| | carboxamide | | |
| | P2 | | |
| 480 | 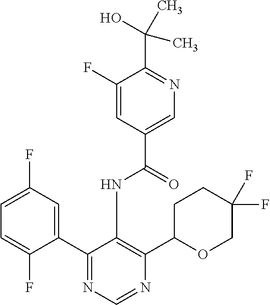 [SMILES: CC(C)(O)c1ncc(C(=O)Nc2c(-c3cc(F)ccc3F)ncnc2C2CCC(F)(F)CO2)cc1F] | column: DAICEL CHIRALPAK IC (250 mm × 30 mm, 10 μm) eluting with 25% of methanol containing 0.1% ammonium hydroxide in supercritical carbon dioxide | 1.432 min, 99%; (400 MHz, CDCl₃) δ 9.16 (s, 1H), 8.98 (s, 1H), 8.70 (s, 1H), 7.76 (d, J = 10.4 Hz, 1H), 7.48-7.39 (m, 1H), 7.13-7.07 (m, 1H), 7.07-6.99 (m, 1H), 5.29 (s, 1H), 4.83 (dd, J = 2.8, 10.0 Hz, 1H), 4.23-4.10 (m, 1H), 3.83-3.67 (m, 1H), 2.43- 2.31 (m, 1H), 2.28-2.20 (m, 1H), 2.19-2.01 (m, 2H), 1.61 (s, 6H); MS (ES+) m/z 509.0 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-5-fluoro-6-(2- | | |
| | hydroxypropan-2- | | |
| | yl)nicotinamide | | |
| | P1 | | |
| 481 | 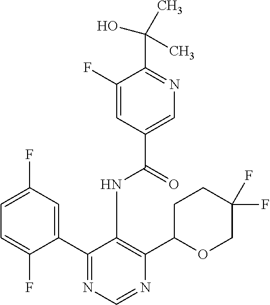 [SMILES: CC(C)(O)c1ncc(C(=O)Nc2c(-c3cc(F)ccc3F)ncnc2C2CCC(F)(F)CO2)cc1F] | column: DAICEL CHIRALPAK IC (250 mm × 30 mm, 10 μm) eluting with 25% of methanol containing 0.1% ammonium hydroxide in supercritical carbon dioxide | 1.637 min, 99%; (400 MHz, CDCl₃) δ 9.16 (s, 1H), 8.98 (s, 1H), 8.70 (s, 1H), 7.76 (dd, J = 1.2, 10.4 Hz, 1H), 7.50-7.37 (m, 1H), 7.12- 7.07 (m, 1H), 7.07-7.01 (m, 1H), 5.29 (s, 1H), 4.83 (dd, J = 2.8, 10.4 Hz, 1H), 4.27- 4.07 (m, 1H), 3.81-3.69 (m, 1H), 2.42-2.36 (m, 1H), 2.27-2.20 (m, 1H), 2.19- 1.98 (m, 2H), 1.61 (s, 6H); MS (ES+) m/z 509.0 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-5-fluoro-6-(2- | | |
| | hydroxypropan-2- | | |
| | yl)nicotinamide | | |
| | P2 | | |
| 482 | 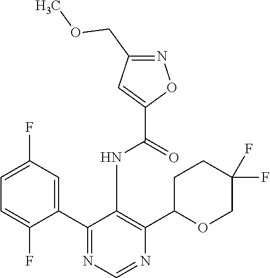 | column: CHIRALPAK IC (150 mm × 4.6 mm) eluting with methanol in supercritical carbon dioxide | 2.364 min, 99%; (400 MHz, DMSO-d₆) δ 10.87 (s, 1H), 9.31 (s, 1H), 739-7.36 (m, 3H), 7.15 (s, 1H), 4.99-4.96 (m, 1H), 4.55 (s, 2H), 3.96- 3.89 (m, 1H), 3.84-3.73 (m, 1H), 3.33 (s, 3H), 2.34-2.11 (m, 3H), 2.09-2.01 (m, 1H); MS (ES+) m/z 467.2 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-3- | | |
| | (methoxymethyl)isoxazole- | | |
| | 5-carboxamide | | |
| | P1 | | |
| 483 | 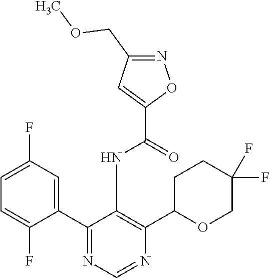 | column: CHIRALPAK IC (150 mm × 4.6 mm) eluting with methanol in supercritical carbon dioxide | 2.624 min, 99%; (400 MHz, DMSO-d₆) δ 10.87 (d, J = 0.2 Hz, 1H), 9.31 (s, 1H), 7.39- 7.36 (m, 3H), 7.15 (s, 1H), 4.97 (dd, J = 9.9, 2.2 Hz, 1H), 4.54 (s, 2H), 3.96-3.89 (m, 1H), 3.84-3.73 (m, 1H), 3.33 (s, 3H), 2.34-2.12 (m, 3H), 2.09-2.01 (m, 1H); MS (ES+) m/z 467.2 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-3- | | |
| | (methoxymethyl)isoxazole- | | |
| | 5-carboxamide | | |
| | P2 | | |
| 484 | 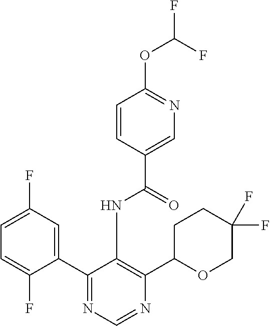 [SMILES: O=C(Nc1c(-c2cc(F)ccc2F)ncnc1C1CCC(F)(F)CO1)c1ccc(OC(F)F)nc1] | column: CHIRALPAK IC (150 mm × 4.6 mm) eluting with methanol containing 0.1% ammonium hydroxide in supercritical carbon dioxide | 2.260 min, 99%; (400 MHz, DMSO-d₆) δ 10.45 (s, 1H), 9.30 (s, 1H), 8.65 (d, J = 2.3 Hz, 1H), 8.22 (dd, J = 8.6, 2.5 Hz, 1H), 7.79 (t, J = 72.3 Hz, 1H), 7.39-7.35 (m, 3H), 7.23 (d, J = 8.5 Hz, 1H), 4.99-4.96 (m, 1H), 3.98-3.91 (m, 1H), 3.85-3.74 (m, 1H), 2.34-2.13 (m, 3H), 2.08-2.01 (m, 1H); MS (ES+) m/z 499.2 (M + 1) |
| | 6-(difluoromethoxy)-N-(4- | | |
| | (2,5-difluorophenyl)-6-(5,5- | | |
| | difluorotetrahydro-2H- | | |
| | pyran-2-yl)pyrimidin-5- | | |
| | yl)nicotinamide | | |
| | P1 | | |
| 485 | 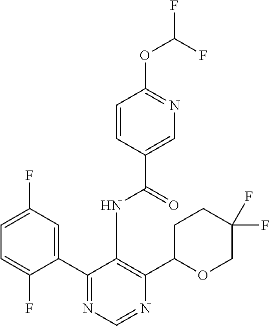 [SMILES: O=C(Nc1c(-c2cc(F)ccc2F)ncnc1C1CCC(F)(F)CO1)c1ccc(OC(F)F)nc1] | column: CHIRALPAK IC (150 mm × 4.6 mm) eluting with methanol containing 0.1% ammonium hydroxide in supercritical carbon dioxide | 2.389 min, 99%; (400 MHz, DMSO-d₆) δ 10.45 (s, 1H), 9.30 (s, 1H), 8.65 (d, J = 2.3 Hz, 1H), 8.22 (dd, J = 8.6, 2.4 Hz, 1H), 7.79 (t, J = 72.3 Hz, 1H), 7.39-7.35 (m, 3H), 7.23 (d, J = 8.6 Hz, 1H), 4.99-4.96 (m, 1H), 3.98-3.91 (m, 1H), 3.85-3.74 (m, 1H), 2.35-2.12 (m, 3H), 2.08-2.00 (m, 1H); MS (ES+) m/z 499.2 (M + 1) |
| | 6-(difluoromethoxy)-N-(4- | | |
| | (2,5-difluorophenyl)-6-(5,5- | | |
| | difluorotetrahydro-2H- | | |
| | pyran-2-yl)pyrimidin-5- | | |
| | yl)nicotinamide | | |
| | P2 | | |
| 486 | 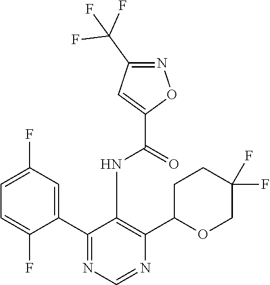 [SMILES: O=C(Nc1c(-c2cc(F)ccc2F)ncnc1C1CCC(F)(F)CO1)c1cc(C(F)(F)F)no1] | column: CHIRALPAK IC (150 mm × 4.6 mm) eluting with methanol in supercritical carbon dioxide | 1.683 min, 99%; (400 MHz, DMSO-d₆) δ 11.18 (d, J = 0.4 Hz, 1H), 9.33 (s, 1H), 7.84 (s, 1H), 7.41-7.37 (m, 3H), 5.01-4.98 (m, 1H), 3.97- 3.90 (m, 1H), 3.85-3.74 (m, 1H), 2.34-2.11 (m, 3H), 2.08- 2.00 (m, 1H); MS (ES+) m/z 491.2 (M + 1) |
| | N-(4-(2,5-difluorophenyl)- | | |
| | 6-(5,5-difluorotetrahydro- | | |
| | 2H--pyran-2-yl)pyrimidin-5- | | |
| | yl)-3- | | |
| | (trifluoromethyl)isoxazole- | | |
| | 5-carboxamide | | |
| | P1 | | |
| 487 | 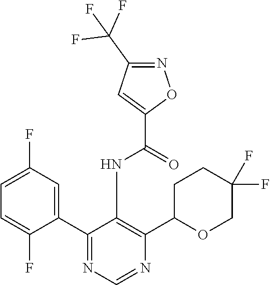 [SMILES: O=C(Nc1c(-c2cc(F)ccc2F)ncnc1C1CCC(F)(F)CO1)c1cc(C(F)(F)F)no1] | column: CHIRALPAK IC (150 mm × 4.6 mm) eluting with methanol in supercritical carbon dioxide | 1.981 min, 99%; (400 MHz, DMSO-d₆) δ 11.18 (s, 1H), 9.33 (s, 1H), 7.84 (s, 1H), 7.43-7.35 (m, 3H), 5.01-4.98 (m, 1H), 3.97-3.90 (m, 1H), 3.85-3.74 (m, 1H), 2.34-2.11 (m, 3H), 2.09-2.00 (m, 1H); MS (ES+) m/z 491.2 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-3- | | |
| | (trifluoromethyl)isoxazole- | | |
| | 5-carboxamide | | |
| | P2 | | |
| 488 | 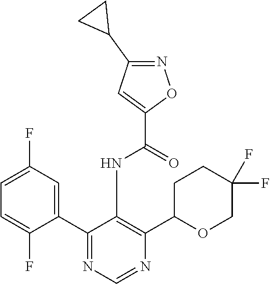 | column: LUXCEL-2 (250 mm × 4.6 mm) eluting with methanol in supercritical carbon dioxide | 3.064 min, 99%; (400 MHz, DMSO-d₆) δ 10.80 (s, 1H), 9.30 (s, 1H), 7.39-7.35 (m, 3H), 6.92 (s, 1H), 4.97-4.94 (m, 1H), 3.96-3.89 (m, 1H), 3.83-3.72 (m, 1H), 2.33-1.99 (m, 5H), 1.11-1.00 (m, 2H), 0.87-0.76 (m, 2H); MS (ES+) m/z 463.2 (M + 1) |
| | 3-cyclopropyl-N-(4-(2,5- | | |
| | difluorophenyl)-6-(5,5- | | |
| | difluorotetrahydro-2H- | | |
| | pyran-2-yl)pyrimidin-5- | | |
| | yl)isoxazole-5- | | |
| | carboxamide | | |
| | P1 | | |
| 489 | 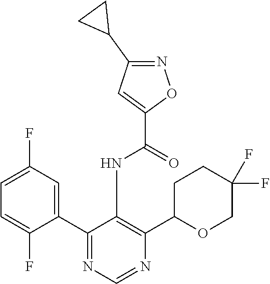 | column: LUXCEL-2 (250 mm × 4.6 mm) eluting with methanol in supercritical carbon dioxide | 3.520 min, 99%; (400 MHz, DMSO-d₆) δ 10.80 (s, 1H), 9.30 (s, 1H), 7.39-7.35 (m, 3H), 6.92 (s, 1H), 4.97-4.94 (m, 1H), 3.96-3.89 (m, 1H), 3.83-3.73 (m, 1H), 2.33-2.00 (m, 5H), 1.08-1.03 (m, 2H), 0.84-0.80 (m, 2H); MS (ES+) m/z 463.2 (M + 1) |
| | 3-cyclopropyl-N-(4-(2,5- | | |
| | difluorophenyl)-6-(5,5- | | |
| | difluorotetrahydro-2H- | | |
| | pyran-2-yl)pyrimidin-5- | | |
| | yl)isoxazole-5- | | |
| | carboxamide | | |
| | P2 | | |
| 490 | 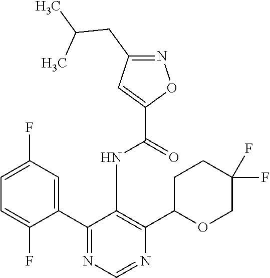 | column: LUXCEL-2 (250 mm × 4.6 mm) eluting with methanol in supercritical carbon dioxide | 2.817 min, 99%; (400 MHz, DMSO-d₆) δ 10.80 (s, 1H), 9.31 (s, 1H), 7.37 (dd, J = 7.5, 5.4 Hz, 3H), 7.04 (s, 1H), 4.99-4.96 (m, 1H), 3.96- 3.89 (m, 1H), 3.84-3.74 (m, 1H), 2.56 (d, J = 7.1 Hz, 2H), 2.34-2.12 (m, 3H), 2.08-2.01 (m, 1H), 1.95 (dq, J = 13.5, 6.7 Hz, 1H), 0.91 (d, J = 6.6 Hz, 6H); MS (ES+) m/z 479.2 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-3-isobutylisoxazole-5- | | |
| | carboxamide | | |
| | P1 | | |
| 491 | 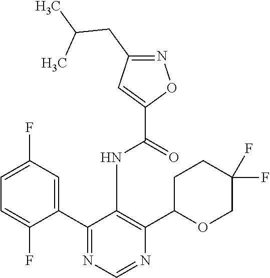 | column: LUXCEL-2 (250 mm × 4.6 mm) eluting with methanol in supercritical carbon dioxide | 3.188 min, 99%; (400 MHz, DMSO-d₆) δ 10.80 (s, 1H), 9.31 (s, 1H), 7.39-7.35 (m, 3H), 7.04 (s, 1H), 4.99-4.96 (m, 1H), 3.96-3.89 (m, 1H), 3.84-3.74 (m, 1H), 2.56 (d, J = 7.1 Hz, 2H), 2.34-2.12 (m, 3H), 2.09-2.01 (m, 1H), 1.94 (tt, J = 13.5, 6.7 Hz, 1H), 0.91 (d, J = 6.6 Hz, 6H); MS (ES+) m/z 479.2 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-3-isobutylisoxazole-5- | | |
| | carboxamide | | |
| | P2 | | |
| 492 | 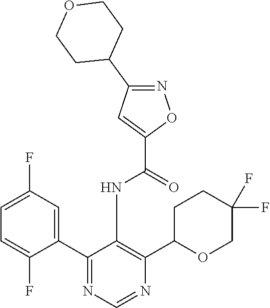 | column: LUXCEL-2 (250 mm × 4.6 mm) eluting with methanol in supercritical carbon dioxide | 3.543 min, 99%; (400 MHz, DMSO-d₆) δ 10.84 (d, J = 0.3 Hz, 1H), 9.30 (s, 1H), 7.37 (t, J = 6.6 Hz, 3H), 7.16 (s, 1H), 4.99-4.96 (m, 1H), 3.96-3.89 (m, 3H), 3.85-3.74 (m, 1H), 3.44 (td, J = 11.6, 2.0 Hz, 2H), 3.06 (tt, J = 11.6, 3.8 Hz, 1H), 2.33-2.11 (m, 3H), 2.06-2.02 (m, 1H), 1.84 (dd, J = 12.7, 1.7 Hz, 2H), 1.67 (qd, J = 12.2, 4.0 Hz, 2H); MS (ES+) m/z 507.2 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-3-(tetrahydro-2H-pyran- | | |
| | 4-yl)isoxazole-5- | | |
| | carboxamide | | |
| | P1 | | |
| 493 | 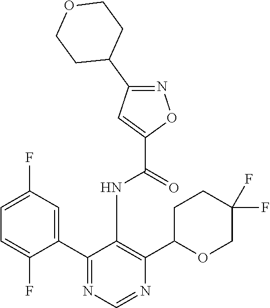 [SMILES: O=C(Nc1c(-c2cc(F)ccc2F)ncnc1C1CCC(F)(F)CO1)c1cc(C2CCOCC2)no1] | column: LUXCEL-2 (250 mm × 4.6 mm) eluting with methanol in supercritical carbon dioxide | 4.163 min, 99%; (400 MHz, DMSO-d₆) δ 10.84 (s,1H), 9.30 (s, 1H), 7.37 (t, J = 6.7 Hz, 3H), 7.16 (d, J = 0.3 Hz, 1H), 4.98-4.95 (m, 1H), 3.96- 3.87 (m, 3H), 3.85-3.74 (m, 1H), 3.44 (td, J = 11.6, 2.0 Hz, 2H), 3.09-3.02 (m, 1H), 2.34-2.10 (m, 3H), 2.07-2.01 (m, 1H), 1.86-1.82 (m, 2H), 1.72-1.62 (m, 2H); MS (ES+) m/z 507.2 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-3-(tetrahydro-2H-pyran- | | |
| | 4-yl)isoxazole-5- | | |
| | carboxamide | | |
| | P2 | | |
| 494 | 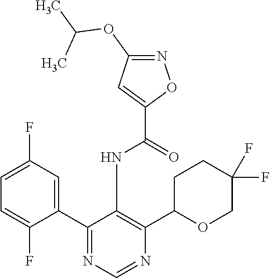 | column: DAICEL CHIRALPAK IC (250 mm × 30 mm, 5 μm) eluting with 25% ethanol containing 0.1% ammonium formate in supercritical carbon dioxide | 0.824 min, 99%; (400 MHz, CDCl₃) δ 9.18 (s, 2H), 7.39 (ddd, J = 2.8, 5.2, 8.0 Hz, 1H), 7.15-7.04 (m, 2H), 6.41 (s, 1H), 4.94 (td, J = 6.4, 12.4 Hz, 1H), 4.78 (dd, J = 4.0, 9.6 Hz, 1H), 4.27-4.17 (m, 1H), 3.84-3.69 (m, 1H), 2.50-2.35 (m, 1H), 2.33- 2.19 (m, 2H), 2.18-1.98 (m, 1H), 1.41 (d, J = 6.0 Hz, 6H); MS (ES+) m/z 481.1 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-3-isopropoxyisoxazole- | | |
| | 5-carboxamide | | |
| | P1 | | |
| 495 | 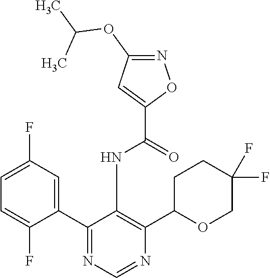 [SMILES: CC(C)Oc1cc(C(=O)Nc2c(-c3cc(F)ccc3F)ncnc2C2CCC(F)(F)CO2)on1] | column: DAICEL CHIRALPAK IC (250 mm × 30 mm, 5 μm) eluting with 25% ethanol containing 0.1% ammonium formate in supercritical carbon dioxide | 1.119 min, 99%; (400 MHz, CDCl₃) δ 9.27-9.11 (m, 2H), 7.40 (ddd, J = 2.8, 5.2, 8.0 Hz, 1H), 7.17-7.04 (m, 2H), 6.42 (s, 1H), 5.10-4.89 (m, 1H), 4.85-4.71 (m, 1H), 4.32-4.11 (m, 1H), 3.86- 3.68 (m, 1H), 2.58-2.37 (m, 1H), 2.35-2.19 (m, 2H), 2.18-1.98 (m, 1H), 1.42 (d, J = 6.0 Hz, 6H); MS (ES+) m/z 481.1 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-3-isopropoxyisoxazole- | | |
| | 5-carboxamide | | |
| | P2 | | |
| 496 | 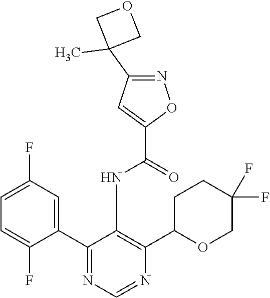 | column: LUXCEL-2 (250 mm × 4.6 mm) eluting with 20% methanol containing 0.1% ammonium formate in supercritical carbon dioxide | 3.008 min, 99%; (400 MHz, DMSO-d₆) δ 10.92 (s, 1H), 9.29 (t, J = 0.2 Hz, 1H), 7.39- 7.36 (m, 3H), 7.29 (m, 1H), 4.99-4.96 (m, 1H), 4.79-4.78 (m, 2H), 4.54 (d, J = 6.0 Hz, 2H), 3.97-3.90 (m, 1H), 3.84- 3.73 (m, 1H), 2.34-2.11 (m, 3H), 2.09-2.02 (m, 1H), 1.67 (s, 3H); MS (ES+) m/z 493.2 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-3-(3-methyloxetan-3- | | |
| | yl)isoxazole-5- | | |
| | carboxamide | | |
| | P1 | | |
| 497 | 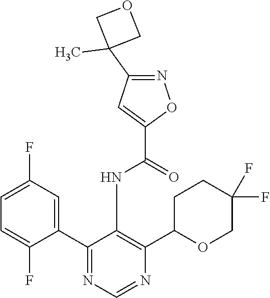 | column: LUXCEL-2 (250 mm × 4.6 mm) eluting with 20% methanol containing 0.1% ammonium formate in supercritical carbon dioxide | 3.353 min, 99%; (400 MHz, DMSO-d₆) δ 10.95 (s, 1H), 9.25 (s, 1H), 7.40-7.34 (m, 3H), 7.26 (m, 1H), 4.98-4.96 (m, 1H), 4.79 (dd, J = 5.9, 0.9 Hz, 2H), 4.54 (d, J = 6.0 Hz, 2H), 3.97-3.90 (m, 1H), 3.83-3.72 (m, 1H), 2.34-2.10 (m, 3H), 2.09-2.03 (m, 1H), 1.67 (s, 3H); MS (ES+) m/z 493.2 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-3-(3-methyloxetan-3- | | |
| | yl)isoxazole-5- | | |
| | carboxamide | | |
| | P2 | | |
| 498 | 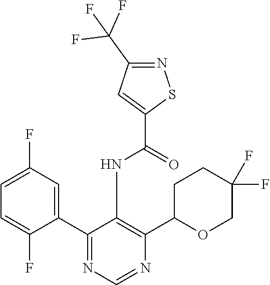 [SMILES: O=C(Nc1c(-c2cc(F)ccc2F)ncnc1C1CCC(F)(F)CO1)c1cc(C(F)(F)F)ns1] | column: DAICEL CHIRALPAK IC (250 mm × 30 mm, 5 μm) eluting with 15% isopropanol containing 0.1% ammonium formate in supercritical carbon dioxide | 0.703 min, 96%; (400 MHz, CDCl₃) δ 9.16 (s, 1H), 8.99 (s, 1H), 7.74 (s, 1H), 7.46- 7.40 (m, 1H), 7.14-7.02 (m, 2H), 4.86-4.80 (m, 1H), 4.21-4.14 (m, 1H), 3.82- 3.70 (m, 1H), 2.45-2.33 (m, 1H), 2.27-2.05 (m, 3H); MS (ES+) m/z 506.9 (M + 1), Rt = 0.703 min |
| | N-(4-(2,5-difluorophenyl)- | | |
| | 6-(5,5-difluorotetrahydro- | | |
| | 2H-pyran-2-yl)pyrimidin-5- | | |
| | yl)-3- | | |
| | (trifluoromethyl)isothiazole- | | |
| | 5-carboxamide | | |
| | P1 | | |
| 499 | 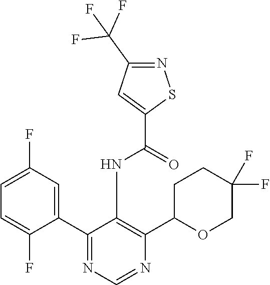 [SMILES: O=C(Nc1c(-c2cc(F)ccc2F)ncnc1C1CCC(F)(F)CO1)c1cc(C(F)(F)F)ns1] | column: DAICEL CHIRALPAK IC (250 mm × 30 mm, 5 μm) eluting with 15% isopropanol containing 0.1% ammonium formate in supercritical carbon dioxide | 0.848 min, 98%; (400 MHz, CDCl₃) δ 9.16 (s, 1H), 9.00 (s, 1H), 7.74 (s, 1H), 7.49- 7.39 (m, 1H), 7.16-7.01 (m, 2H), 4.83 (dd, J = 2.8, 10.4 Hz, 1H), 4.25-4.10 (m, 1H), 3.76 (td, J = 12.4, 17.2 Hz, 1H), 2.45-2.33 (m, 1H), 2.27-2.05 (m, 3H); MS (ES+) m/z 506.9 (M + 1), Rt = 0.848 min |
| | N-(4-(2,5-difluorophenyl)- | | |
| | 6-(5,5-difluorotetrahydro- | | |
| | H-pyran-2-yl)pyrimidin-5- | | |
| | yl)-3- | | |
| | (trifluoromethyl)isothiazole- | | |
| | 5-carboxamide | | |
| | P2 | | |
| 500 | 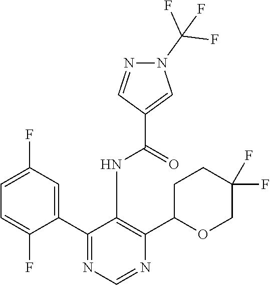 [SMILES: O=C(Nc1c(-c2cc(F)ccc2F)ncnc1C1CCC(F)(F)CO1)c1cnn(C(F)(F)F)c1] | column: LUXCEL-2 Cellulose (250 mm × 10 mm) eluting with 5% methanol containing 0.1% ammonium hydroxide in supercritical carbon dioxide | 2.307 min, 99%; (400 MHz, DMSO-d₆) δ 10.31 (s, 1H), 9.29 (s, 1H), 9.05 (s, 1H), 8.35 (s, 1H), 7.36 (t, J = 6.5 Hz, 3H), 4.96-4.93 (m, 1H), 3.99-3.92 (m, 1H), 3.85-3.74 (m, 1H), 2.35-2.11 (m, 3H), 2.07-1.96 (m, 1H); MS (ES+) m/z 490.2 (M + 1), Rt = 2.307 min |
| | N-(4-(2,5-difluorophenyl)- | | |
| | 6-(5,5-difluorotetrahydro- | | |
| | 2H-pyran-2-yl)pyrimidin-5- | | |
| | yl)-1-(trifluoromethyl)-lH- | | |
| | pyrazole-4-carboxamide | | |
| | P1 | | |
| 501 | 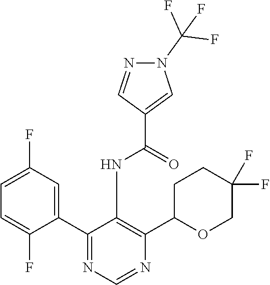 [SMILES: O=C(Nc1c(-c2cc(F)ccc2F)ncnc1C1CCC(F)(F)CO1)c1cnn(C(F)(F)F)c1] | column: LUXCEL-2 Cellulose (250 mm × 10 mm) eluting with 5% methanol containing 0.1% ammonium hydroxide in supercritical carbon dioxide | 2.407 min, 96%; (400 MHz, DMSO-d₆) δ 10.32 (s, 1H), 9.29 (s, 1H), 9.05 (s, 1H), 8.35 (s, 1H), 7.36 (t, J = 6.4 Hz, 3H), 4.96-4.93 (m, 1H), 3.99-3.92 (m, 1H), 3.85-3.74 (m, 1H), 2.34-2.12 (m, 3H), 2.06-1.97 (m, 1H); MS (ES+) m/z 490.2 (M + 1) |
| | N-(4-(2,5-difluorophenyl)- | | |
| | 6-(5,5-difluorotetrahydro-- | | |
| | 2H-pyran-2-yl)pyrimidin-5- | | |
| | yl)-1-(trifluoromethyl)-lH- | | |
| | pyrazole-4-carboxamide | | |
| | P2 | | |

### Example 502

### Synthesis of 3-(2,2-difluoroethoxy)-N-(4-(2,5-difluorophenyl)-6-(3-methyl-4-oxocyclohexyl)pyrimidin-5-yl)isoxazole-5-carboxamide

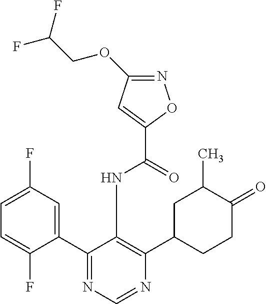 [SMILES: CC1CC(c2ncnc(-c3cc(F)ccc3F)c2NC(=O)c2cc(OCC(F)F)no2)CCC1=O]

### Step 1. Preparation of tert-butyl (4-(2,5-difluorophenyl)-6-(3-methyl-4-oxocyclohexyl)pyrimidin-5-yl)carbamate

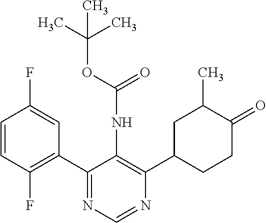 [SMILES: CC1CC(c2ncnc(-c3cc(F)ccc3F)c2NC(=O)OC(C)(C)C)CCC1=O]

To a vial containing tert-butyl (4-chloro-6-(2,5-difluorophenyl)pyrimidin-5-yl)carbamate (0.15 g, 0.44 mmol) was added 3-methyl-4-oxocyclohexanecarboxylic acid (0.21 g, 1.3 mmol), (4,4″-di-t-butyl-2,2″-bipyridine)bis[3,5-difluoro-2-[5-trifluoromethyl-2-pyridinyl-kN)phenyl-kC]iridium(II) hexafluorophosphate (0.0050 g, 0.0044 mmol), dichloro(dimethoxyethane)nickel (0.0096 g, 0.044 mmol), 4-tert-butyl-2-(4-tert-butyl-2-pyridyl)pyridine (0.018 g, 0.066 mmol), cesium carbonate (0.44 g, 1.4 mmol), and N,N-dimethylformamide (11 mL). The vial was sealed, and the reaction mixture was stirred in front of Kessil PR160L lights (440 nm) for 20 h. The reaction mixture was diluted with ethyl acetate (25 mL) and washed with saturated ammonium chloride solution (2×50 mL), water (50 mL), and brine (50 mL). The organic layer was dried over anhydrous magnesium sulfate, filtered, and concentrated in vacuo. The resulting residue was used as is in the next step without further purification (0.18 g, 99% yield): MS (ES+) m/z 418.4 (M+1).

### Step 2. Preparation of 4-(5-amino-6-(2,5-difluorophenyl)pyrimidin-4-yl)-2-methylcyclohexan-1-one

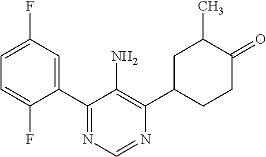 [SMILES: CC1CC(c2ncnc(-c3cc(F)ccc3F)c2N)CCC1=O]

To a mixture of tert-butyl (4-(2,5-difluorophenyl)-6-(3-methyl-4-oxocyclohexyl)pyrimidin-5-yl)carbamate (0.18 g, 0.44 mmol) in 1,4-dioxane (0.88 mL) was added 4 M hydrogen chloride in 1,4-dioxane (1.1 mL, 4.4 mmol). The reaction mixture was stirred for 20 h. The reaction mixture was diluted with ethyl acetate (20 mL) and washed with saturated ammonium hydroxide solution (2×20 mL), and brine (20 mL). The organic layer was dried over anhydrous magnesium sulfate, filtered, and concentrated in vacuo. The resulting residue was used as is in the next step without further purification (0.14 g, quant. yield): MS (ES+) m/z 318.3 (M+1).

### Step 3. Preparation of 3-(2,2-difluoroethoxy)-N-(4-(2,5-difluorophenyl)-6-(3-methyl-4-oxocyclohexyl)pyrimidin-5-yl)isoxazole-5-carboxamide

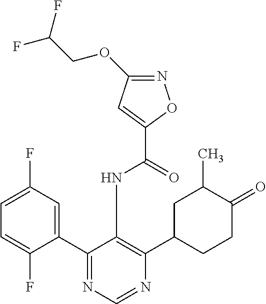 [SMILES: CC1CC(c2ncnc(-c3cc(F)ccc3F)c2NC(=O)c2cc(OCC(F)F)no2)CCC1=O]

To a mixture of 4-(5-amino-6-(2,5-difluorophenyl)pyrimidin-4-yl)-2-methylcyclohexan-1-one (0.14 g, 0.44 mmol), 3-(2,2-difluoroethoxy)isoxazole-5-carboxylic acid (0.13 g, 0.66 mmol) and 2-chloro-1-methylpyridinium iodide (0.34 g, 1.3 mmol) and N,N-diisopropylethylamine (0.46 mL, 2.6 mmol) added anhydrous tetrahydrofuran (2.9 mL). The reaction mixture was stirred at 68° C. for 3 h. After cooling to ambient temperature, the mixture was diluted with methanol (5 mL) and 1M sodium hydroxide (2 mL). The mixture was stirred at ambient temperature for 30 min before it was diluted with ethyl acetate (20 mL). The organic layer was washed with saturated ammonium chloride solution (2×30 mL), dried over anhydrous magnesium sulfate, filtered, and concentrated in vacuo. Purification of the residue by column chromatography, eluting with a gradient of 5 to 60% ethyl acetate in heptane, afforded the title compound a colorless solid (0.070 g, 31% yield): ¹H-NMR (400 MHz; DMSO-d₆) δ 11.00 (s, 1H), 9.22 (s, 1H), 7.37 (dtd, J=12.8, 7.2, 2.1 Hz, 3H), 7.08 (s, 1H), 6.43 (tt, J=53.8, 3.1 Hz, 1H), 4.59 (ddd, J=16.6, 13.4, 3.2 Hz, 2H), 3.69-3.62 (m, 1H), 2.77-2.56 (m, 2H), 2.31-2.27 (m, 1H), 2.13-2.10 (m, 2H), 2.04-1.93 (m, 1H), 1.76 (q, J=12.6 Hz, 1H), 0.93 (d, J=6.5 Hz, 3H); MS (ES+) m/z 493.2 (M+1).

### Example 503 and 504

### Synthesis of 3-(3,3-difluorocyclobutoxy)-N-(4-(2,5-difluorophenyl)-6-(5,5-difluorotetrahydro-2H-pyran-2-yl)pyrimidin-5-yl)isoxazole-5-carboxamide P1/P2

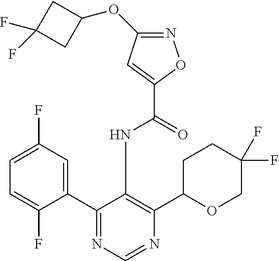 [SMILES: O=C(Nc1c(-c2cc(F)ccc2F)ncnc1C1CCC(F)(F)CO1)c1cc(OC2CC(F)(F)C2)no1]

### Step 1. Preparation of methyl 3-(3,3-difluorocyclobutoxy)isoxazole-5-carboxylate

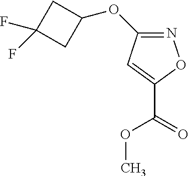 [SMILES: COC(=O)c1cc(OC2CC(F)(F)C2)no1]

Methyl 3-hydroxyisoxazole-5-carboxylate (0.20 g, 1.4 mmol) was dissolved in anhydrous tetrahydrofuran (14 mL), followed by 3,3-difluorocyclobutan-1-ol (0.23 g, 2.1 mmol) and triphenylphosphine (0.48 g, 1.8 mmol). To the mixture was added diisopropylazodicarboxylate (0.36 mL, 1.8 mmol) dropwise. After stirring for 15 minutes at ambient temperature under a nitrogen atmosphere, the reaction mixture was stirred at 50° C. for 16 h. Purification of the residue by column chromatography, eluting with a gradient of 0 to 60% ethyl acetate in heptane, afforded the title compound as an off-white solid (0.18 g, 56% yield): ¹H-NMR (400 MHz; DMSO-d₆) δ 7.17 (s, 1H), 5.04-4.95 (m, 1H), 3.89 (s, 3H), 3.23-3.12 (m, 2H), 2.90-2.79 (m, 2H); MS (ES+) m/z 234.2 (M+1)

### Step 2. Preparation of 3-(3,3-difluorocyclobutoxy)isoxazole-5-carboxylic acid

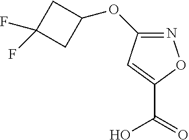 [SMILES: O=C(O)c1cc(OC2CC(F)(F)C2)no1]

To a mixture of methyl 3-(3,3-difluorocyclobutoxy)isoxazole-5-carboxylate (0.45 g, 1.9 mmol) in tetrahydrofuran (1.9 mL) and methanol (1.9 mL) was added 5M sodium hydroxide solution (0.58 mL) in water (1.9 mL). After stirring at ambient temperature for 8 h, the reaction mixture was diluted with ethyl acetate (20 mL) and water (20 mL). The aqueous phase was acidified with 1 M hydrochloric acid solution and extracted with ethyl acetate (2×25 mL). The combined organic layers were dried over anhydrous magnesium sulfate, filtered, and concentrated in vacuo. The resulting residue was used as is in the next step without further purification (0.50 g, quant. yield): ¹H-NMR (400 MHz; DMSO-d₆) δ 6.14 (s, 1H), 4.93-4.84 (m, 1H), 3.20-3.07 (m, 2H), 2.83-2.69 (m, 2H); MS (ES+) m/z 220.2 (M+1).

### Step 3. Preparation of 3-(3,3-difluorocyclobutoxy)-N-(4-(2,5-difluorophenyl)-6-(5,5-difluorotetrahydro-2H-pyran-2-yl)pyrimidin-5-yl)isoxazole-5-carboxamide

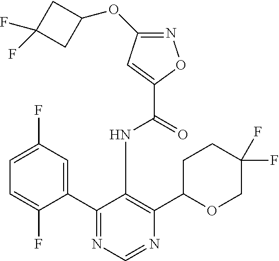 [SMILES: O=C(Nc1c(-c2cc(F)ccc2F)ncnc1C1CCC(F)(F)CO1)c1cc(OC2CC(F)(F)C2)no1]

To a mixture of 4-(2,5-difluorophenyl)-6-(5,5-difluorotetrahydro-2H-pyran-2-yl)pyrimidin-5-amine (0.17 g, 0.47 mmol), 3-(3,3-difluorocyclobutoxy)isoxazole-5-carboxylic acid (0.16 g, 0.71 mmol) and 2-chloro-1-methylpyridinium iodide (0.36 g, 1.4 mmol) and N,N-diisopropylethylamine (0.49 mL, 2.8 mmol) added anhydrous tetrahydrofuran (4.7 mL). The reaction mixture was stirred at 68° C. for 20 h. After cooling to ambient temperature, the mixture was diluted with methanol (5 mL) and 1M sodium hydroxide (2 mL). The mixture was stirred at ambient temperature for 1 h before it was diluted with ethyl acetate (20 mL). The organic layer was washed with saturated ammonium chloride solution (2×30 mL), dried over anhydrous magnesium sulfate, filtered, and concentrated in vacuo. Purification of the residue by column chromatography, eluting with a gradient of 20 to 95% ethyl acetate in heptane, afforded the title compound a colorless solid (0.087 g, 35% yield): ¹H-NMR (400 MHz; DMSO-d₆) δ 10.85 (d, J=0.2 Hz, 1H), 9.31 (s, 1H), 7.42-7.34 (m, 3H), 6.98 (s, 1H), 5.04-4.92 (m, 2H), 3.97-3.91 (m, 1H), 3.84-3.74 (m, 1H), 3.24-3.11 (m, 2H), 2.90-2.76 (m, 2H), 2.34-2.10 (m, 3H), 2.08-1.99 (m, 1H); MS (ES+) m/z 529.2 (M+1).

### Step 4. Preparation of 3-(3,3-difluorocyclobutoxy)-N-(4-(2,5-difluorophenyl)-6-(5,5-difluorotetrahydro-2H-pyran-2-yl)pyrimidin-5-yl)isoxazole-5-carboxamide P1/P2

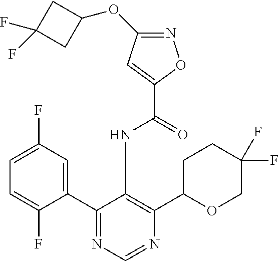 [SMILES: O=C(Nc1c(-c2cc(F)ccc2F)ncnc1C1CCC(F)(F)CO1)c1cc(OC2CC(F)(F)C2)no1]

3-(3,3-difluorocyclobutoxy)-N-(4-(2,5-difluorophenyl)-6-(5,5-difluorotetrahydro-2H-pyran-2-yl)pyrimidin-5-yl)isoxazole-5-carboxamide (0.070 g, 0.13 mmol) was purified by chiral SFC (column: Lux Cel-2 (250 mm×4.6 mm), eluting with 5% to 60% methanol containing in supercritical carbon dioxide, to afford peak 1 (retention time=2.651 min) as a colorless solid (0.030 g, 43% yield, 99% ee): ¹H NMR (400 MHz, DMSO-d₆) δ 10.84 (s, 1H), 9.31 (s, 1H), 7.43-7.34 (m, 3H), 6.98 (s, 1H), 5.03-4.93 (m, 2H), 3.97-3.90 (m, 1H), 3.84-3.73 (m, 1H), 3.23-3.11 (m, 2H), 2.90-2.75 (m, 2H), 2.34-2.11 (m, 3H), 2.08-1.99 (m, 1H); MS (ES+) m/z 529.2 (M+1).

Peak 2 (retention time=2.965 min) afforded a colorless solid (0.030 g, 43% yield, 99% ee): ¹H NMR (400 MHz, DMSO-d₆) δ 10.84 (s, 1H), 9.31 (s, 1H), 7.42-7.35 (m, 3H), 6.97 (s, 1H), 5.03-4.94 (m, 2H), 3.97-3.90 (m, 1H), 3.84-3.73 (m, 1H), 3.24-3.11 (m, 2H), 2.90-2.76 (m, 2H), 2.35-2.11 (m, 3H), 2.07-1.99 (m, 1H); MS (ES+) m/z 529.2 (M+1).

In a similar manner as described in examples disclosed herein, utilizing the appropriately substituted starting materials, the following intermediates were prepared:

| Structure | Amount | | |
|---|---|---|---|
| Name | Yield | MS (ES+) m/z | ¹H NMR |
| 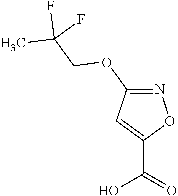 | 0.56 g Quant. | 208.2 (M + 1) | (400 MHz, DMSO-d₆) δ 7.10-7.08 (m, 1H), 4.57 (t, J = 13.1 Hz, 2H), 1.73 (t, J = 19.3 Hz, 3H) |
| difluoropropoxy)isoxazole- | | | |
| 5-carboxylic acid | | | |
| 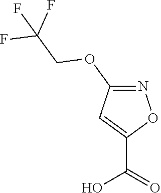 [SMILES: O=C(O)c1cc(OCC(F)(F)F)no1] | 0.48 g Quant. | 212.2 (M + 1) | (400 MHz, DMSO-d₆) δ 6.94 (s, 1H), 5.00 (q, J = 8.8 Hz, 2H). |
| 3-(2,2,2- | | | |
| trifluoroethoxy)isoxazole- | | | |
| 5-carboxylic acid | | | |
| 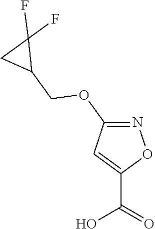 [SMILES: O=C(O)c1cc(OCC2CC2(F)F)no1] | 0.36 g Quant. | 220.2 (M + 1) | (400 MHz, DMSO-d₆) δ 6.14 (s, 1H), 4.34-4.28 (m, 1H), 4.11 (ddd, J = 11.0, 8.9, 1.9 Hz, 1H), 2.32-2.20 (m, 1H), 1.77-1.68 (m, 1H), 1.58-1.49 (m, 1H). |
| 3-((2,2- | | | |
| difluorocycloprop- | | | |
| yl)methoxy)isoxazole- | | | |
| 5-carboxylic acid | | | |
| 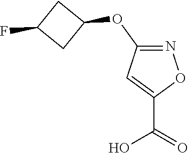 [SMILES: O=C(O)c1cc(O[C@H]2C[C@@H](F)C2)no1] | 0.27 g Quant. | 202.2 (M + 1) | (400 MHz, DMSO-d₆) δ 6.13 (s, 1H), 4.85 (dquintet, J = 56.3, 6.4 Hz, 1H), 4.53-4.45 (m, 1H), 3.00-2.92 (m, 2H), 2.33-2.20 (m, 2H), 1.64 (s, 1H) |
| 3-((1R,3R or | | | |
| 1S,3S)-3- | | | |
| fluorocyclobutoxy)isoxazole-5- | | | |
| carboxylic acid | | | |
| 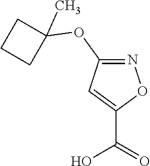 | 0.17g Quant. | 198.2 (M + 1) | (400 MHz, DMSO-d₆) δ 5.97 (s, 1H), 2.35-2.27 (m, 2H), 2.13-2.07 (m, 2H), 1.79-1.71 (m, 1H), 1.71-1.65 (m, 1H), 1.54 (s, 3H) |
| 3-(1- | | | |
| methylcyclo- | | | |
| butoxy)isoxazole-5- | | | |
| carboxylic acid | | | |
| 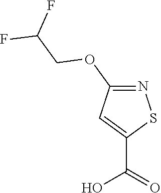 [SMILES: O=C(O)c1cc(OCC(F)F)ns1] | 0.28 g 98% | 210.0 (M + 1) | (400 MHz, DMSO-d₆) δ 6.69 (s, 1H), 6.38 (tt, J = 54.6, 3.5 Hz, 1H), 4.53 (td, J = 15.0, 3.5 Hz, 2H). |
| 3-(2,2- | | | |
| difluoroethoxy)isothiazole-5- | | | |
| carboxylic acid | | | |
| 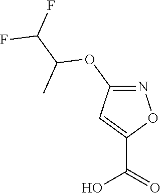 | 0.63 g Quant. | 208.0 (M + 1) | (400 MHz, DMSO-d₆) δ 6.37-6.09 (m, 2H), 4.91 (tqd, J = 12.1, 6.1, 2.8 Hz, 1H), 1.64 (s, 1H), 1.35 (d, J = 6.5 Hz, 3H) |
| 3-((1,1- | | | |
| difluoropropan-2- | | | |
| yl)oxy)isoxazole- | | | |
| 5-carboxylic acid | | | |

In a similar manner as described in examples disclosed herein, utilizing the appropriately substituted starting materials and intermediates, the following compounds were prepared:

| Example | Structure | Amount | | |
|---|---|---|---|---|
| No. | Name | Yield | MS (ES+) m/z | ¹H NMR |
| 505 | 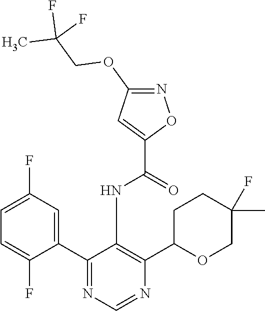 | 0.11 g 56% | 517.2 (M + 1) | (400 MHz, DMSO-d₆) δ 10.89 (s, 1H), 9.31 (s, 1H), 7.40-7.36 (m, 3H), 7.04 (s, 1H), 4.98-4.95 (m, 1H), 4.57 (dd, J = 13.1 Hz, 2H), 3.97-3.91 (m, 1H), 3.85-3.74 (m, 1H), 2.34-2.11 (m, 3H), 2.05-2.01 (m, 1H), 1.73 (t, J = 19.3 Hz, 3H) |
| | N-(4-(2,5- | | | |
| | difluorophenyl)-6-(5,5- | | | |
| | difluorotetrahydro-2H- | | | |
| | pyran-2-yl)pyrimidin-5- | | | |
| | yl)-3-(2,2- | | | |
| | difluoropropoxy)isoxazole- | | | |
| | 5-carboxamide | | | |
| 506 | 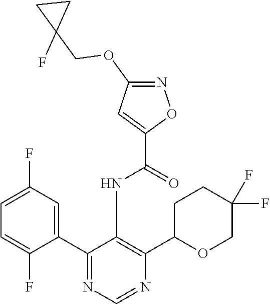 [SMILES: O=C(Nc1c(-c2cc(F)ccc2F)ncnc1C1CCC(F)(F)CO1)c1cc(OCC2(F)CC2)no1] | 0.083 g 39% | 511.2 (M + 1) | (400 MHz, DMSO-d₆) δ 10.84 (s, 1H), 9.32 (s, 1H), 7.40-7.35 (m, 3H), 7.00 (s, 1H), 4.97-4.95 (m, 1H), 4.56 (d, J = 23.0 Hz, 2H), 3.97-3.91 (m, 1H), 3.85-3.74 (m, 1H), 2.34-2.11 (m, 3H), 2.08-2.01 (m, 1H), 1.19-1.11 (m, 2H), 0.93 (q, J = 7.3 Hz, 2H) |
| | N-(4-(2,5- | | | |
| | difluorophenyl)-6-(5,5- | | | |
| | difluorotetrahydro-2H- | | | |
| | pyran-2-yl)pyrimidin-5- | | | |
| | yl)-3-((1- | | | |
| | fluorocyclopropyl)methoxy)isoxazole-5- | | | |
| | carboxamide | | | |
| 507 | 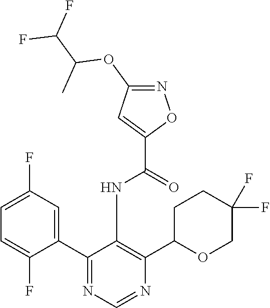 | 0.065 g 35% | 517.2 (M + 1) | (400 MHz, DMSO-d₆) δ 10.86 (s, 1H), 9.32 (s, 1H), 7.40-7.35 (m, 3H), 7.03 (s, 1H), 6.28 (td, J = 54.4, 2.7 Hz, 1H), 5.08-4.95 (m, 2H), 3.97-3.91 (m, 1H), 3.80 (dt, J = 21.5, 10.9 Hz, 1H), 2.34-2.12 (m, 3H), 2.08-2.00 (m, 1H), 1.39 (d, J = 6.5 Hz, 3H) |
| | N-(4-(2,5- | | | |
| | difluorophenyl)-6-(5,5- | | | |
| | difluorotetrahydro-2H- | | | |
| | pyran-2-yl)pyrimidin-5- | | | |
| | yl)-3-((1,1- | | | |
| | difluoropropan-2- | | | |
| | yl)oxy)isoxazole-5- | | | |
| | carboxamide | | | |
| 508 | 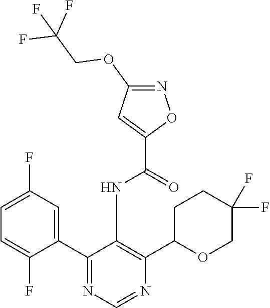 [SMILES: O=C(Nc1c(-c2cc(F)ccc2F)ncnc1C1CCC(F)(F)CO1)c1cc(OCC(F)(F)F)no1] | 0.087 g 46% | 521.2 (M + 1) | (400 MHz, DMSO-d₆) δ 10.91 (s, 1H), 9.32 (s, 1H), 7.38 (ddd, J = 7.7, 5.5, 2.7 Hz, 3H), 7.12 (s, 1H), 5.04 (q, J = 8.7 Hz, 2H), 4.98-4.95 (m, 1H), 3.97-3.91 (m, 1H), 3.85-3.74 (m, 1H), 2.33-2.11 (m, 3H), 2.03 (t, J = 8.3 Hz, 1H) |
| | N-(4-(2,5- | | | |
| | difluorophenyl)-6-(5,5- | | | |
| | difluorotetrahydro-2H- | | | |
| | pyran-2-yl)pyrimidin-5- | | | |
| | yl)-3-(2,2,2- | | | |
| | trifluoroethoxy)isoxazole- | | | |
| | 5-carboxamide | | | |
| 509 | 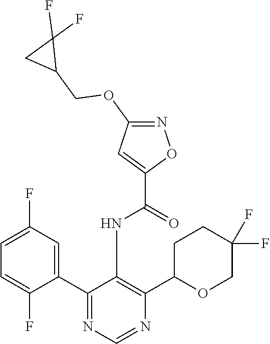 | 0.035 g 16% | 529.2 (M + 1) | (400 MHz, DMSO-d₆) δ 10.84 (d, J = 0.4 Hz, 1H), 9.31 (s, 1H), 7.40- 7.35 (m, 3H), 6.97 (s, 1H), 4.97-4.94 (m, 1H), 4.45-4.40 (m, 1H), 4.23-4.18 (m, 1H), 3.97-3.91 (m, 1H), 3.79 (dt, J = 21.6, 10.9 Hz, 1H), 2.35-2.26 (m, 2H), 2.24-2.11 (m, 2H), 2.05-2.00 (m, 1H), 1.81-1.73 (m, 1H), 1.63-1.55 (m, 1H) |
| | 3-((2,2- | | | |
| | difluorocyclopropyl)methoxy)- | | | |
| | N-(4-(2,5- | | | |
| | difluorophenyl)-6-(5,5- | | | |
| | difluorotetrahydro-2H- | | | |
| | pyran-2-yl)pyrimidin-5- | | | |
| | yl)isoxazole-5- | | | |
| | carboxamide | | | |
| 510 | 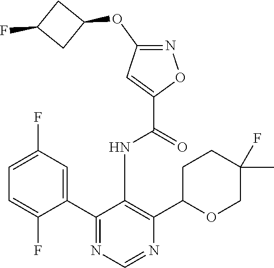 | 0.065 g 30% | 511.2 (M + 1) | (400 MHz, DMSO-d₆) δ 10.83 (s, 1H), 9.31 (s, 1H), 7.40-7.35 (m, 3H), 6.92 (s, 1H), 4.98-4.92 (m, 1H), 4.84-4.78 (m, 1H), 4.63-4.55 (m, 1H), 3.98-3.89 (m, 1H), 3.85-3.74 (m, 1H), 3.04-2.94 (m, 2H), 2.40-2.10 (m, 5H), 2.07-1.98 (m, 1H), |
| | N-(4-(2,5- | | | |
| | difluorophenyl)-6-(5,5- | | | |
| | difluorotetrahydro-2H- | | | |
| | pyran-2-yl)pyrimidin-5- | | | |
| | yl)-syn-3- | | | |
| | fluorocyclobutoxy)-isoxazole- | | | |
| | 5-carboxamide | | | |
| 511 | 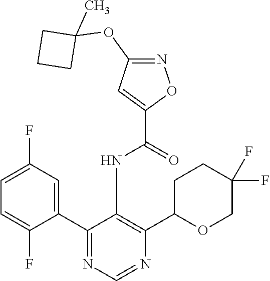 [SMILES: CC1(Oc2cc(C(=O)Nc3c(-c4cc(F)ccc4F)ncnc3C3CCC(F)(F)CO3)on2)CCC1] | 0.045 g 24% | 507.2 (M + 1) | (400 MHz, DMSO-d₆) δ 10.78 (s, 1H), 9.30 (s, 1H), 7.38 (q, J = 6.7 Hz, 3H), 6.80 (d, J = 0.2 Hz, 1H), 4.97-4.95 (m, 1H), 3.97-3.90 (m, 1H), 3.86-3.75 (m, 1H), 2.38-2.11 (m, 7H), 2.04-2.00 (m, 1H), 1.81-1.72 (m, 1H), 1.70-1.63 (m, 1H), 1.58 (s, 3H) |
| | N-(4-(2,5- | | | |
| | difluorophenyl)-6-(5,5- | | | |
| | difluorotetrahydro-2H- | | | |
| | pyran-2-yl)pyrimidin-5- | | | |
| | yl)-3-(1- | | | |
| | methylcyclobutoxy)isoxazole- | | | |
| | 5-carboxamide | | | |
| 512 | 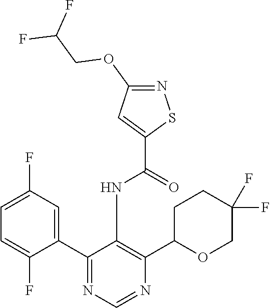 [SMILES: O=C(Nc1c(-c2cc(F)ccc2F)ncnc1C1CCC(F)(F)CO1)c1cc(OCC(F)F)ns1] | 0.068 g 32% | 519.2 (M + 1) | (400 MHz, DMSO-d₆) δ 10.73 (s, 1H), 9.32 (s, 1H), 7.45 (s, 1H), 7.40- 7.36 (m, 3H), 6.57-6.28 (m, 1H), 4.98-4.96 (m, 1H), 4.64 (td, J = 15.1, 3.3 Hz, 2H), 3.99-3.92 (m, 1H), 3.81 (dt, J = 21.4, 10.9 Hz, 1H), 2.34-2.13 (m, 3H), 2.07-1.98 (m, 1H) |
| | 3-(2,2-difluoroethoxy)-N- | | | |
| | (4-(2,5-difluorophenyl)- | | | |
| | 6-(5,5- | | | |
| | difluorotetrahydro-2H- | | | |
| | pyran-2-yl)pyrimidin-5- | | | |
| | yl)isothiazole-5- | | | |
| | carboxamide | | | |

In a similar manner as described in examples disclosed herein, utilizing the appropriate chiral separation conditions, the following compounds were prepared:

| Example | Structure | SFC | Retention time (Rₜ), ee; ¹H NMR; |
|---|---|---|---|
| No. | Name | Conditions | MS (ES+) m/z |
| 513 | 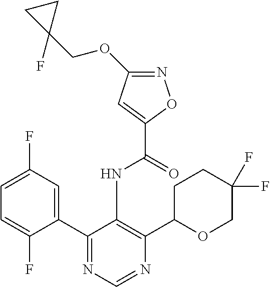 [SMILES: O=C(Nc1c(-c2cc(F)ccc2F)ncnc1C1CCC(F)(F)CO1)c1cc(OCC2(F)CC2)no1] | column: LUX CEL-2 (250 mm × 10 mm) eluting with 30% methanol containing 0.1% ammonium hydroxide in supercritical carbon dioxide | 2.673 min, 99%; (400 MHz, DMSO-d₆) δ 10.85 (s, 1H), 9.30 (s, 1H), 7.40-7.35 (m, 3H), 6.99 (s, 1H), 4.97-4.95 (m, 1H), 4.55 (d, J = 23.0 Hz, 2H), 3.98-3.91 (m, 1H), 3.84-3.74 (m, 1H), 2.33-2.12 (m, 3H), 2.07-2.00 (m, 1H), 1.19-1.11 (m, 2H), 0.95- 0.90 (m, 2H); MS (ES+) m/z 511.2 (M + 1) |
| | N-(4-(2,5-difluorophenyl)- | | |
| | 6-(5,5-difluorotetrahydro- | | |
| | 2H-pyran-2-yl)pyrimidin- | | |
| | 5-yl)-3-((1- | | |
| | fluorocyclopropyl)methoxy) | | |
| | isoxazole-5- | | |
| | carboxamide | | |
| | P1 | | |
| 514 | 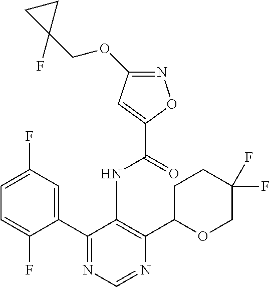 [SMILES: O=C(Nc1c(-c2cc(F)ccc2F)ncnc1C1CCC(F)(F)CO1)c1cc(OCC2(F)CC2)no1] | column: LUX CEL-2 (250 mm × 10 mm) eluting with 30% methanol containing 0.1% ammonium hydroxide in supercritical carbon dioxide | 3.012 min, 99%; (400 MHz, DMSO-d₆) δ 10.85 (s, 1H), 9.30 (s, 1H), 7.39-7.35 (m, 3H), 6.98 (s, 1H), 4.96 (dd, J = 9.9, 2.0 Hz, 1H), 4.55 (d, J = 23.0 Hz, 2H), 3.98-3.91 (m, 1H), 3.84-3.73 (m, 1H), 2.34-2.11 (m, 3H), 2.09- 2.01 (m, 1H), 1.15 (dt, J = 18.7, 7.0 Hz, 2H), 0.95-0.90 (m, 2H) |
| | N-(4-(2,5-difluorophenyl)- | | |
| | 6-(5,5-difluorotetrahydro- | | |
| | 2H-pyran-2-yl)pyrimidin- | | |
| | 5-yl)-3-((1- | | |
| | fluorocyclopropyl)methoxy) | | |
| | isoxazole-5- | | |
| | carboxamide | | |
| | P2 | | |
| 515 | 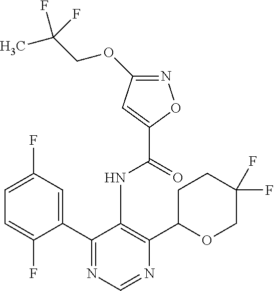 [SMILES: CC(F)(F)COc1cc(C(=O)Nc2c(-c3cc(F)ccc3F)ncnc2C2CCC(F)(F)CO2)on1] | column: LUX CEL-2 (250 mm × 4.6 mm) eluting with 10% methanol containing 0.1% ammonium hydroxide in supercritical carbon dioxide | 2.391 min, 99%; (400 MHz, DMSO-d₆) δ 10.92 (s, 1H), 9.30 (s, 1H), 7.39-7.36 (m, 3H), 7.03 (s, 1H), 4.97-4.95 (m, 1H), 4.57 (t, J = 13.1 Hz, 2H), 3.97-3.91 (m, 1H), 3.84-3.73 (m, 1H), 2.34-2.11 (m, 3H), 2.08-2.01 (m, 1H), 1.73 (t, J = 19.3 Hz, 3H); MS (ES+) m/z 517.2 (M + 1) |
| | N-(4-(2,5-difluorophenyl)- | | |
| | 6-(5,5-d ifluorotetrahydro- | | |
| | 2H-pyran-2-yl)pyrimidin- | | |
| | 5-yl)-3-(2,2- | | |
| | difluoropropoxy)isoxazole- | | |
| | 5-carboxamide | | |
| | P1 | | |
| 516 | 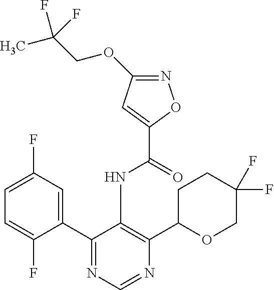 [SMILES: CC(F)(F)COc1cc(C(=O)Nc2c(-c3cc(F)ccc3F)ncnc2C2CCC(F)(F)CO2)on1] | column: LUX CEL-2 (250 mm × 4.6 mm) eluting with 10% methanol containing 0.1% ammonium hydroxide in supercritical carbon dioxide | 2.690 min, 98%; (400 MHz, DMSO-d₆) δ 9.23 (s, 1H), 7.37 (td, J = 6.0, 4.5 Hz, 3H), 6.99- 6.97 (m, 1H), 4.97-4.95 (m, 1H), 4.59-4.52 (m, 2H), 3.97-3.90 (m, 1H), 3.83-3.72 (m, 1H), 2.30- 2.12 (m, 3H), 2.09-2.01 (m, 1H), 1.73 (t, J = 19.3 Hz, 3H); MS (ES+) m/z 517.2 (M + 1) |
| | N-(4-(2,5-difluorophenyl)- | | |
| | 6-(5,5-difluorotetrahydro- | | |
| | 2H-pyran-2-yl)pyrimidin- | | |
| | 5-yl)-3-(2,2- | | |
| | difluoropropoxy)isoxazole- | | |
| | 5-carboxamide | | |
| | P2 | | |
| 517 | 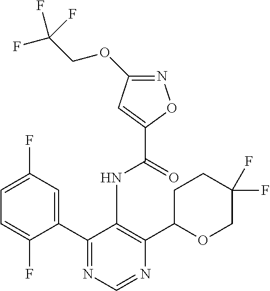 [SMILES: O=C(Nc1c(-c2cc(F)ccc2F)ncnc1C1CCC(F)(F)CO1)c1cc(OCC(F)(F)F)no1] | column: LUX CEL-2 (250 mm × 4.6 mm) eluting with 7% methanol containing 0.1% ammonium hydroxide in supercritical carbon dioxide | 2.180 min, 99%; (400 MHz, DMSO-d₆) δ 10.95 (s, 1H), 9.29 (s, 1H), 7.38 (t, J = 6.3 Hz, 3H), 7.09 (s, 1H), 5.03 (q, J = 8.7 Hz, 2H), 4.97-4.95 (m, 1H), 3.97- 3.90 (m, 1H), 3.84-3.73 (m, 1H), 2.34-2.13 (m, 3H), 2.09-2.00 (m, 1H); MS (ES+) m/z 521.2 (M + 1) |
| | N-(4-(2,5-difluorophenyl)- | | |
| | 6-(5,5-difluorotetrahydro- | | |
| | 2H-pyran-2-yl)pyrimidin- | | |
| | 5-yl)-3-(2,2,2- | | |
| | trifluoroethoxy)isoxazole- | | |
| | 5-carboxamide | | |
| | P1 | | |
| 518 | 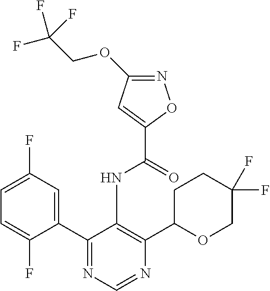 [SMILES: O=C(Nc1c(-c2cc(F)ccc2F)ncnc1C1CCC(F)(F)CO1)c1cc(OCC(F)(F)F)no1] | column: LUX CEL-2 (250 mm × 4.6 mm) eluting with 7% methanol containing 0.1% ammonium hydroxide in supercritical carbon dioxide | 2.435 min, 99%; (400 MHz, DMSO-d₆) δ 10.97 (s, 1H), 9.29 (s, 1H), 7.38 (t, J = 6.3 Hz, 3H), 7.09 (s, 1H), 5.02 (t, J = 8.7 Hz, 2H), 4.97 (dd, J = 7.1, 3.0 Hz, 1H), 3.97-3.91 (m, 1H), 3.84- 3.73 (m, 1H), 2.35-2.11 (m, 3H), 2.05-2.01 (m, 1H); MS (ES+) m/z 521.2 (M + 1) |
| | N-(4-(2,5-difluorophenyl)- | | |
| | 6-(5,5-difluorotetrahydro- | | |
| | 2H-pyran-2-yl)pyrimidin- | | |
| | 5-yl)-3-(2,2,2- | | |
| | trifluoroethoxy)isoxazole- | | |
| | 5-carboxamide | | |
| | P2 | | |
| 519 | 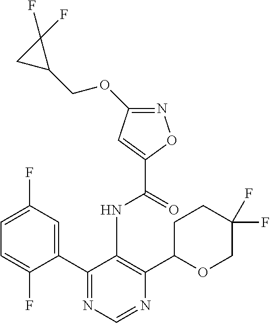 [SMILES: O=C(Nc1c(-c2cc(F)ccc2F)ncnc1C1CCC(F)(F)CO1)c1cc(OCC2CC2(F)F)no1] | column: LUX CEL-2 (250 mm × 10 mm) eluting with 15% methanol containing 0.1% ammonium hydroxide in supercritical carbon dioxide | 2.535 min, 99%; (400 MHz, DMSO-d₆) δ 10.79 (s, 1H), 9.25 (s, 1H), 7.37 (t, J = 6.6 Hz, 3H), 6.89 (t, J = 0.5 Hz, 1H), 4.96- 4.93 (m, 1H), 4.42 (ddd, J = 10.8, 7.3, 3.0 Hz, 1H), 4.23-4.18 (m, 1H), 3.97-3.90 (m, 1H), 3.83-3.72 (m, 1H), 2.35-2.10 (m, 4H), 2.08-2.00 (m, 1H), 1.81- 1.71 (m, 1H), 1.63-1.54 (m, 1H); MS (ES+) m/z 529.2 (M + 1) |
| | 3-((2,2- | | |
| | difluorocyclopropyl)methoxy)- | | |
| | N-(4-(2,5- | | |
| | difluorophenyl)-6-(5,5- | | |
| | difluorotetrahydro-2H- | | |
| | pyran-2-yl)pyrimidin-5- | | |
| | yl)isoxazole-5- | | |
| | carboxamide | | |
| | P1 | | |
| 520 | 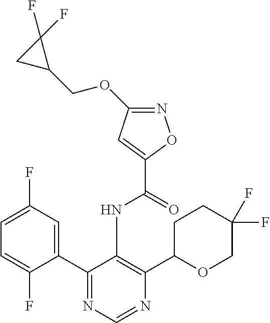 [SMILES: O=C(Nc1c(-c2cc(F)ccc2F)ncnc1C1CCC(F)(F)CO1)c1cc(OCC2CC2(F)F)no1] | column: LUX CEL-2 (250 mm × 10 mm) eluting with 15% methanol containing 0.1% ammonium hydroxide in supercritical carbon dioxide | 2.845 min, 99%; (400 MHz, DMSO-d₆) δ 10.91 (s, 1H), 9.26 (s, 1H), 7.37 (t, J = 6.6 Hz, 3H), 6.90 (t, J = 0.3 Hz, 1H), 4.96- 4.94 (m, 1H), 4.42 (ddd, J = 10.7, 7.2, 3.1 Hz, 1H), 4.23-4.18 (m, 1H), 3.99-3.89 (m, 1H), 3.78 (dt, J = 21.7, 10.9 Hz, 1H), 2.35- 2.13 (m, 4H), 2.10-2.00 (m, 1H), 1.81-1.72 (m, 1H), 1.63-1.55 (m, 1H); MS (ES+) m/z 529.2 (M + 1) |
| | 3-((2,2- | | |
| | difluorocyclopropyl)methoxy)- | | |
| | N-(4-(2,5- | | |
| | difluorophenyl)-6-(5,5- | | |
| | difluorotetrahydro-2H- | | |
| | pyran-2-yl)pyrimidin-5- | | |
| | yl)isoxazole-5- | | |
| | carboxamide | | |
| | P2 | | |
| 521 | 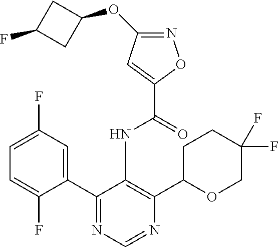 [SMILES: O=C(Nc1c(-c2cc(F)ccc2F)ncnc1C1CCC(F)(F)CO1)c1cc(O[C@H]2C[C@@H](F)C2)no1] | column: LUX CEL-2 (250 mm × 10 mm) eluting with 15% methanol containing 0.1% ammonium hydroxide in supercritical carbon dioxide | 2.736 min, 99%; (400 MHz, DMSO-d₆) δ 10.82 (s, 1H), 9.30 (s, 1H), 7.40-7.35 (m, 3H), 6.91 (s, 1H), 4.98-4.92 (m, 1H), 4.81 (quintet, J = 6.4 Hz, 1H), 4.59 (tdt, J = 6.4, 6.1, 5.4 Hz, 1H), 3.97-3.90 (m, 1H), 3.85-3.74 (m, 1H), 3.04-2.95 (m, 2H), 2.39- 2.25 (m, 3H), 2.24-2.00 (m, 3H); MS (ES+) m/z 511.2 (M + 1), Rt = 2.736 min |
| | N-(4-(2,5-difluorophenyl)- | | |
| | 6-(5,5-difluorotetrahydro- | | |
| | 2H-pyran-2-yl)pyrimidin- | | |
| | 5-yl)-3-(syn-3- | | |
| | fluorocyclobutoxy) | | |
| | isoxazole-5-carboxamide | | |
| | P1 | | |
| 522 | 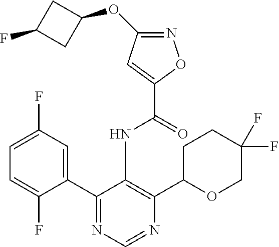 [SMILES: O=C(Nc1c(-c2cc(F)ccc2F)ncnc1C1CCC(F)(F)CO1)c1cc(O[C@H]2C[C@@H](F)C2)no1] | column: LUX CEL-2 (250 mm × 10 mm) eluting with 15% methanol containing 0.1% ammonium hydroxide in supercritical carbon dioxide | 3.102 min, 99%; (400 MHz, DMSO-d₆) δ 10.84 (s, 1H), 9.29 (s, 1H), 7.39-7.35 (m, 3H), 6.90 (s, 1H), 4.96 (q, J = 6.3 Hz, 1H), 4.81 (quintet, J = 6.4 Hz, 1H), 4.62-4.54 (m, 1H), 3.97-3.90 (m, 1H), 3.84-3.73 (m, 1H), 3.04- 2.95 (m, 2H), 2.40-2.11 (m, 5H), 2.08-2.00 (m, 1H); MS (ES+) m/z 511.2 (M + 1), Rₜ = 3.102 min |
| | N-(4-(2,5-difluorophenyl)- | | |
| | 6-(5,5-difluorotetrahydro- | | |
| | 2H-pyran-2-yl)pyrimidin- | | |
| | 5-yl)-3-(syn-3- | | |
| | fluorocyclobutoxy) | | |
| | isoxazole-5-carboxamide | | |
| | P2 | | |
| 523 | 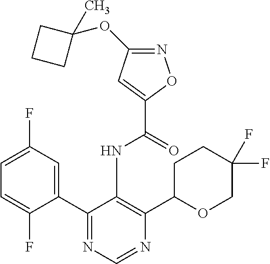 [SMILES: CC1(Oc2cc(C(=O)Nc3c(-c4cc(F)ccc4F)ncnc3C3CCC(F)(F)CO3)on2)CCC1] | column: LUX CEL-2 (250 mm × 10 mm) eluting with 20% methanol containing 0.1% ammonium hydroxide in supercritical carbon dioxide | 2.811 min, 99%; (400 MHz, DMSO-d₆) δ 10.79 (s, 1H), 9.29 (s, 1H), 7.38 (q, J = 6.6 Hz, 3H), 6.79 (s, 1H), 4.97-4.95 (m, 1H), 3.97-3.91 (m, 1H), 3.80 (dt, J = 21.5, 10.9 Hz, 1H), 2.38-2.26 (m, 3H), 2.24-2.11 (m, 4H), 2.04-2.00 (m, 1H), 1.81-1.72 (m, 1H), 1.67 (dd, J = 19.0, 9.4 Hz, 1H), 1.58 (s, 3H); MS (ES+) m/z 507.2 (M + 1) |
| | N-(4-(2,5-difluorophenyl)- | | |
| | 6-(5,5-difluorotetrahydro- | | |
| | 2H-pyran-2-yl)pyrimidin- | | |
| | 5-yl)-3-(1- | | |
| | methylcyclobutoxy) | | |
| | isoxazole-5-carboxamide | | |
| | P1 | | |
| 524 | 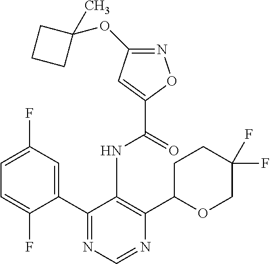 [SMILES: CC1(Oc2cc(C(=O)Nc3c(-c4cc(F)ccc4F)ncnc3C3CCC(F)(F)CO3)on2)CCC1] | column: LUX CEL-2 (250 mm × 10 mm) eluting with 20% methanol containing 0.1% ammonium hydroxide in supercritical carbon dioxide | 3.276 min, 99%; (400 MHz, DMSO-d₆) δ 10.82 (s, 1H), 9.26 (s, 1H), 7.39-7.35 (m, 3H), 6.75 (s, 1H), 4.97-4.94 (m, 1H), 3.97- 3.90 (m, 1H), 3.85-3.74 (m, 1H), 2.38-2.25 (m, 3H), 2.23-2.10 (m, 4H), 2.04-2.01 (m, 1H), 1.81- 1.72 (m, 1H), 1.70-1.63 (m, 1H), 1.58 (s, 3H); MS (ES+) m/z 507.2 (M + 1) |
| | N-(4-(2,5-difluorophenyl)- | | |
| | 6-(5,5-difluorotetrahydro- | | |
| | 2H-pyran-2-yl)pyrimidin- | | |
| | 5-yl)-3-(1- | | |
| | methylcyclobutoxy) | | |
| | isoxazole-5-carboxamide | | |
| | P2 | | |
| 525 | 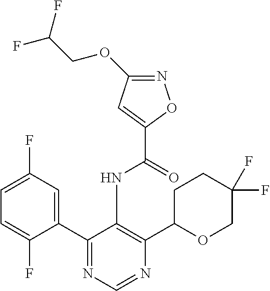 [SMILES: O=C(Nc1c(-c2cc(F)ccc2F)ncnc1C1CCC(F)(F)CO1)c1cc(OCC(F)F)no1] | column: LUX CEL-2 (250 mm × 10 mm) eluting with 10% acetonitrile/ ethanol 1:1 mixture containing 0.1% ammonium hydroxide in supercritical carbon dioxide | 2.551 min, 99%; (400 MHz, DMSO-d₆) δ 10.76 (s, 1H), 9.29 (s, 1H), 7.42 (s, 1H), 7.37 (t, J = 6.6 Hz, 3H), 6.42 (tt, J = 54.2, 3.3 Hz, 1H), 4.98-4.95 (m, 1H), 4.64 (td, J = 15.1, 3.2 Hz, 2H), 3.99-3.92 (m, 1H), 3.80 (dt, J = 21.3, 10.9 Hz, 1H), 2.34-2.12 (m, 3H), 2.07-2.00 (m, 1H); MS (ES+) m/z 519.2 (M + 1) |
| | 3-(2,2-difluoroethoxy)-N- | | |
| | (4-(2,5-difluorophenyl)-6- | | |
| | (5,5-difluorotetrahydro- | | |
| | 2H-pyran-2-yl)pyrimidin- | | |
| | 5-yl)isothiazole-5- | | |
| | carboxamide | | |
| | P1 | | |
| 526 | 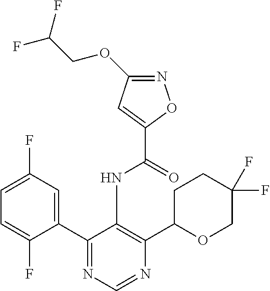 [SMILES: O=C(Nc1c(-c2cc(F)ccc2F)ncnc1C1CCC(F)(F)CO1)c1cc(OCC(F)F)no1] | column: LUX CEL-2 (250 mm × 10 mm) eluting with 10% acetonitrile/ ethanol 1:1 mixture containing 0.1% ammonium hydroxide in supercritical carbon dioxide | 2.685 min, 99%; (400 MHz, DMSO-d₆) δ 10.75 (s, 1H), 9.31 (s, 1H), 7.44 (s, 1H), 7.38 (t, J = 6.5 Hz, 3H), 6.42 (tt, J = 54.2, 3.3 Hz, 1H), 4.98-4.96 (m, 1H), 4.64 (td, J = 15.1, 3.2 Hz, 2H), 3.99-3.92 (m, 1H), 3.86-3.75 (m, 1H), 2.34-2.12 (m, 3H), 2.04- 2.00 (m, 1H); MS (ES+) m/z 519.2 (M + 1) |
| | 3-(2,2-difluoroethoxy)-N- | | |
| | (4-(2,5-difluorophenyl)-6- | | |
| | (5,5-difluorotetrahydro- | | |
| | 2H-pyran-2-yl)pyrimidin- | | |
| | 5-yl)isothiazole-5- | | |
| | carboxamide | | |
| | P2 | | |

### Example 527

### Synthesis of N-(4-(2,5-difluorophenyl)-6-(5,5-difluorotetrahydro-2H-pyran-2-yl)pyrimidin-5-yl)-3-(oxetan-2-ylmethoxy)isoxazole-5-carboxamide

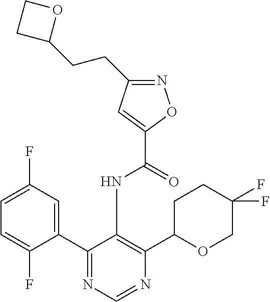 [SMILES: O=C(Nc1c(-c2cc(F)ccc2F)ncnc1C1CCC(F)(F)CO1)c1cc(CCC2CCO2)no1]

### Step 1. Preparation of methyl 3-(oxetan-2-ylmethoxy)isoxazole-5-carboxylate

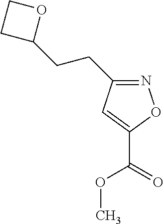

Methyl 3-hydroxyisoxazole-5-carboxylate (0.41 g, 2.9 mmol) was dissolved in N,N-dimethylformamide (14 mL), followed by 2-(iodomethyl)oxetane (0.85 g, 4.3 mmol) and potassium carbonate (0.99 g, 7.2 mmol). After stirring for further 3 h at ambient temperature, the reaction mixture was diluted with ethyl acetate (30 mL), washed with saturated ammonium chloride (2×35 mL), dried over anhydrous magnesium sulfate, filtered, and concentrated in vacuo. Purification of the residue by column chromatography, eluting with 0 to 40% ethyl acetate in heptane, afforded the title compound as a light-yellow solid (0.37 g, 60% yield): MS (ES+) m/z 214.2 (M+1).

### Step 2. Preparation of 3-(oxetan-2-ylmethoxy)isoxazole-5-carboxylic acid

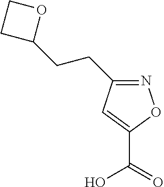 [SMILES: O=C(O)c1cc(CCC2CCO2)no1]

To a mixture of methyl 3-(oxetan-2-ylmethoxy)isoxazole-5-carboxylate (0.37 g, 1.7 mmol) in tetrahydrofuran (1.7 mL) and methanol (1.7 mL) was added 5M sodium hydroxide solution (0.52 mL) in water (1.7 mL). After stirring at ambient temperature for 8 h, the reaction mixture was diluted with ethyl acetate (20 mL) and partitioned with water (2×20 mL). The combined aqueous layers were concentrated in vacuo. The resulting residue was used as is in the next step without further purification (0.31 g, 76% yield): MS (ES+) m/z 200.2 (M+1).

### Step 3. Preparation of N-(4-(2,5-difluorophenyl)-6-(5,5-difluorotetrahydro-2H-pyran-2-yl)pyrimidin-5-yl)-3-(oxetan-2-ylmethoxy)isoxazole-5-carboxamide

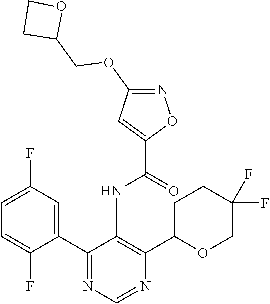 [SMILES: O=C(Nc1c(-c2cc(F)ccc2F)ncnc1C1CCC(F)(F)CO1)c1cc(OCC2CCO2)no1]

To a mixture of 4-(2,5-difluorophenyl)-6-(5,5-difluorotetrahydro-2H-pyran-2-yl)pyrimidin-5-amine hydrochloride (0.10 g, 0.27 mmol), 3-(oxetan-2-ylmethoxy)isoxazole-5-carboxylic acid (0.098 g, 0.41 mmol) and 2-chloro-1-methylpyridinium iodide (0.21 g, 0.82 mmol) and N,N-diisopropylethylamine (0.29 mL, 1.6 mmol) added anhydrous tetrahydrofuran (2.5 mL). The reaction mixture was stirred at 68° C. for 20 h. After cooling to ambient temperature, the mixture was diluted with methanol (2.5 mL) and 5N sodium hydroxide (2.5 mL), and stirred at 80° C. for 1.5 h. The solvent was concentrated in vacuo and partitioned between 1M sodium hydroxide solution (10 mL) and ethyl acetate (15 mL), washed with water (15 mL), dried over anhydrous magnesium sulfate, filtered, and concentrated in vacuo. Purification of the residue by reverse-phased column chromatography, eluting with a gradient of 20 to 95% acetonitrile in water, afforded the title compound a colorless solid (0.0076 g, 5.3% yield): ¹H-NMR (400 MHz; DMSO-d₆) δ 10.86 (s, 1H), 9.28 (s, 1H), 7.37 (t, J=6.0 Hz, 3H), 6.94 (s, 1H), 5.04-4.93 (m, 2H), 4.56-4.32 (m, 5H), 3.97-3.91 (m, 1H), 3.84-3.75 (m, 1H), 2.74-2.65 (m, 1H), 2.30-2.10 (m, 3H), 2.05-2.01 (m, 1H); MS (ES+) m/z 509.2 (M+1).

### Example 528 and 529

### Synthesis of N-(4-(2,5-difluorophenyl)-6-(5,5-difluorotetrahydro-2H-pyran-2-yl)pyrimidin-5-yl)-2-isopropoxypyrimidine-5-carboxamide P1/P2

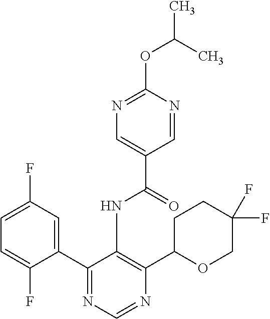 [SMILES: CC(C)Oc1ncc(C(=O)Nc2c(-c3cc(F)ccc3F)ncnc2C2CCC(F)(F)CO2)cn1]

### Step 1. Preparation of 2-chloro-N-(4-(2,5-difluorophenyl)-6-(5,5-difluorotetrahydro-2H-pyran-2-yl)pyrimidin-5-yl)pyrimidine-5-carboxamide

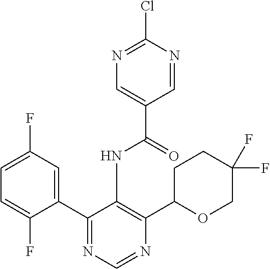 [SMILES: O=C(Nc1c(-c2cc(F)ccc2F)ncnc1C1CCC(F)(F)CO1)c1cnc(Cl)nc1]

To a mixture of 4-(2,5-difluorophenyl)-6-(5,5-difluorotetrahydro-2H-pyran-2-yl)pyrimidin-5-amine (0.19 g, 0.59 mmol), 2-chloropyrimidine-5-carboxylic acid (0.14 g, 0.89 mmol) and 2-chloro-1-methylpyridinium iodide (0.45 g, 1.8 mmol) and N,N-diisopropylethylamine (0.62 mL, 3.5 mmol) added anhydrous tetrahydrofuran (5.9 mL). The reaction mixture was stirred at 68° C. for 18 h. After cooling to ambient temperature, the mixture was diluted with methanol (5 mL) and 1M sodium hydroxide (2 mL) and stirred at ambient temperature for 30 min before it was diluted with ethyl acetate (20 mL). The organic layer was washed with saturated ammonium chloride solution (2×30 mL), dried over anhydrous magnesium sulfate, filtered, and concentrated in vacuo. Purification of the residue by column chromatography, eluting with a gradient of 5 to 70% ethyl acetate in heptane, afforded the title compound as a colorless solid (0.20 g, 71% yield): MS (ES+) m/z 468.4 (M+1) 470.4 (M+1).

### Step 2. Preparation of rac-,N-(4-(2,5-difluorophenyl)-6-(5,5-difluorotetrahydro-2H-pyran-2-yl)pyrimidin-5-yl)-2-isopropoxypyrimidine-5-carboxamide

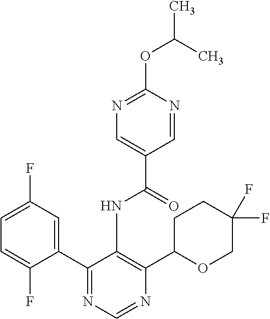 [SMILES: CC(C)Oc1ncc(C(=O)Nc2c(-c3cc(F)ccc3F)ncnc2C2CCC(F)(F)CO2)cn1]

To 2-chloro-N-(4-(2,5-difluorophenyl)-6-(5,5-difluorotetrahydro-2H-pyran-2-yl)pyrimidin-5-yl)pyrimidine-5-carboxamide (0.096 g, 0.20 mmol) was added anhydrous isopropanol (2.0 mL), N,N-dimethylformamide (2.0 mL), and 60% sodium hydride dispersion in mineral oil (0.082 g, 2.0 mmol). The solution was stirred at ambient temperature for 2 h. The reaction mixture was diluted with ethyl acetate (20 mL) and washed with saturated ammonium chloride solution (2×20 mL). The organic layer was dried over anhydrous magnesium sulfate, filtered, and concentrated in vacuo. Purification of the residue by column chromatography, eluting with a gradient of 5 to 80% ethyl acetate in heptane, afforded the title compound as a colorless solid (0.058 g, 53% yield): ¹H-NMR (400 MHz; DMSO-d₆) δ 10.46 (s, 1H), 9.30 (s, 1H), 8.90 (s, 2H), 7.37 (t, J=6.6 Hz, 3H), 5.28 (7, J=6.2 Hz, 1H), 4.97 (dd, J=6.9, 2.9 Hz, 1H), 3.99-3.92 (m, 1H), 3.87-3.76 (m, 1H), 2.33-2.13 (m, 3H), 2.07-1.99 (m, 1H), 1.34 (d, J=6.2 Hz, 6H); MS (ES+) m/z 492.2 (M+1).

### Step 7. Preparation of N-(4-(2,5-difluorophenyl)-6-(5,5-difluorotetrahydro-2H-pyran-2-yl)pyrimidin-5-yl)-2-isopropoxypyrimidine-5-carboxamide P1/P2

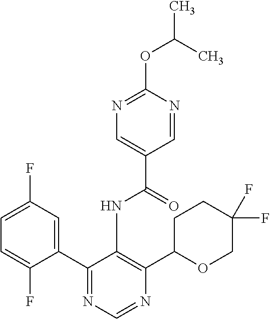 [SMILES: CC(C)Oc1ncc(C(=O)Nc2c(-c3cc(F)ccc3F)ncnc2C2CCC(F)(F)CO2)cn1]

N-(4-(2,5-difluorophenyl)-6-(5,5-difluorotetrahydro-2H-pyran-2-yl)pyrimidin-5-yl)-2-isopropoxypyrimidine-5-carboxamide (0.030 g, 0.060 mmol) was purified by chiral SFC (column: DAICEL CHIRALPAK IG (250 mm×30 mm, 5 μm), eluting with 25% of isopropyl alcohol containing 0.1% ammonium hydroxide in supercritical carbon dioxide, to afford peak 1 (retention time=1.204 min) as a colorless solid (0.0059 g, 24% yield, 99% ee): ¹H NMR (400 MHz, DMSO-d₆) δ 10.44 (s, 1H), 9.29 (s, 1H), 8.89 (s, 2H), 7.49-7.29 (m, 3H), 5.27 (td, J=6.0, 12.4 Hz, 1H), 4.96 (d, J=8.4 Hz, 1H), 3.99-3.90 (m, 1H), 3.87-3.73 (m, 1H), 2.31-2.13 (m, 3H), 2.02 (d, J=10.4 Hz, 1H), 1.34 (d, J=6.4 Hz, 6H); MS (ES+) m/z 492.0 (M+1).

Peak 2 (retention time=1.302 min) afforded a colorless solid (0.0073 g, 23% yield, 96% ee): ¹H NMR (400 MHz, DMSO-d₆) δ 10.44 (s, 1H), 9.29 (s, 1H), 8.89 (s, 2H), 7.40-7.32 (m, 3H), 5.27 (td, J=6.0, 12.4 Hz, 1H), 4.96 (d, J=8.0 Hz, 1H), 3.99-3.91 (m, 1H), 3.87-3.74 (m, 1H), 2.31-2.12 (m, 3H), 2.02 (d, J=10.4 Hz, 1H), 1.34 (d, J=6.0 Hz, 6H); MS (ES+) m/z 492.0 (M+1).

In a similar manner as described in examples disclosed herein, utilizing the appropriately substituted starting materials and intermediates, the following compounds were prepared:

| Example | Structure | Amount | | |
|---|---|---|---|---|
| No. | Name | Yield | MS (ES+) m/z | ¹H NMR |
| 530 | 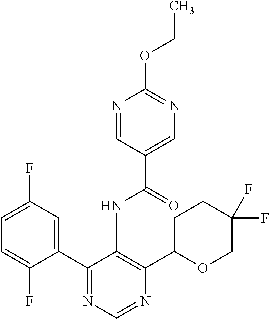 [SMILES: CCOc1ncc(C(=O)Nc2c(-c3cc(F)ccc3F)ncnc2C2CCC(F)(F)CO2)cn1] | 0.084 g 79% | 478.2 (M + 1) | (400 MHz, DMSO-d₆) δ 10.48 (s, 1H), 9.30 (s, 1H), 8.91 (s, 2H), 7.37 (t, J = 6.5 Hz, 3H), 4.99-4.96 (m, 1H), 4.43 (q, J = 7.1 Hz, 2H), 3.99-3.92 (m, 1H), 3.87-3.76 (m, 1H), 2.34-2.13 (m, 3H), 2.07-1.99 (m, 1H), 1.35 (t, J = 7.1 Hz, 3H) |
| | N-(4-(2,5- | | | |
| | difluorophenyl)-6-(5,5- | | | |
| | difluorotetrahydro-2H- | | | |
| | pyran-2-yl)pyrimidin-5- | | | |
| | yl)-2-ethoxypyrimidine-5- | | | |
| | carboxamide | | | |
| 531 | 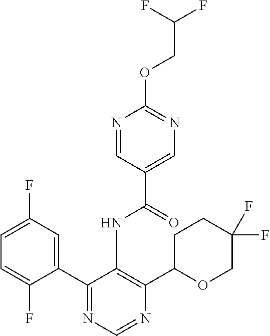 [SMILES: O=C(Nc1c(-c2cc(F)ccc2F)ncnc1C1CCC(F)(F)CO1)c1cnc(OCC(F)F)nc1] | 0.060 g 25% | 514.2 (M + 1) | (400 MHz, DMSO-d₆) δ 10.56 (s, 1H), 9.31 (s, 1H), 8.96 (s, 2H), 7.40- 7.36 (m, 3H), 6.45 (tt, J = 54.3, 3.3 Hz, 1H), 4.99-4.97 (m, 1H), 4.71 (td, J = 15.0, 3.3 Hz, 2H), 3.98-3.92 (m, 1H), 3.87-3.76 (m, 1H), 2.30-2.13 (m, 3H), 2.08-2.02 (m, 1H) |
| | 2-(2,2-difluoroethoxy)-N- | | | |
| | (4-(2,5-difluorophenyl)- | | | |
| | 6-(5,5- | | | |
| | difluorotetrahydro-2H- | | | |
| | pyran-2-yl)pyrimidin-5- | | | |
| | yl)pyrimidine-5- | | | |
| | carboxamide | | | |

In a similar manner as described in examples disclosed herein, utilizing appropriate chiral separation condition, following compounds were prepared:

| | Structure | | |
|---|---|---|---|
| Example | Name | SFC | Retention time (Rt), ee; ¹H NMR; |
| No. | P1 or P2 | Conditions | MS (ES+) m/z |
| 532 | 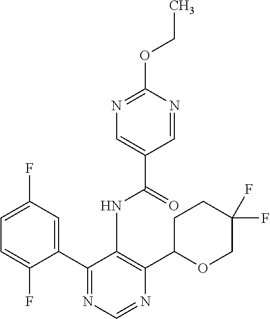 [SMILES: CCOc1ncc(C(=O)Nc2c(-c3cc(F)ccc3F)ncnc2C2CCC(F)(F)CO2)cn1] | DAICEL CHIRALCEL OJ (250 mm × 30 mm, 10 μm) eluting with 15% of methanol containing 0.1% ammonium hydroxide in supercritical carbon dioxide | 0.843 min, 98%; (400 MHz, DMSO-d₆) δ 10.46 (s, 1H), 9.29 (s, 1H), 8.90 (s, 2H), 7.37 (t, J = 6.4 Hz, 3H), 4.96 (d, J = 8.8 Hz, 1H), 4.42 (q, J = 7.2 Hz, 2H), 3.99-3.89 (m, 1H), 3.88-3.73 (m, 1H), 2.32-2.11 (m, 3H), 2.04-2.01 (m, 1H), 1.35 (t, J = 7.2 Hz, 3H); MS (ES+) m/z 478.1 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-2-ethoxypyrimidine-5- | | |
| | carboxamide | | |
| | P1 | | |
| 533 | 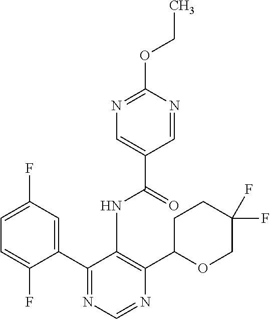 [SMILES: CCOc1ncc(C(=O)Nc2c(-c3cc(F)ccc3F)ncnc2C2CCC(F)(F)CO2)cn1] | DAICEL CHIRALCEL OJ (250 mm × 30 mm, 10 μm) eluting with 15% of methanol containing 0.1% ammonium hydroxide in supercritical carbon dioxide | 0.949 min, 98%; (400 MHz, DMSO-d₆) δ 10.46 (s, 1H), 9.29 (s, 1H), 8.90 (s, 2H), 7.36 (t, J = 6.4 Hz, 3H), 4.96 (d, J = 8.4 Hz, 1H), 4.42 (q, J = 7.2 Hz, 2H), 3.99-3.89 (m, 1H), 3.87-3.73 (m, 1H), 2.31-2.09 (m, 3H), 2.04-2.02 (m, 1H), 1.34 (t, J = 7.2 Hz, 3H); MS (ES+) m/z 478.1 (M + 1) |
| | N-(4-(2,5-difluorophenyl)- | | |
| | 6-(5,5-difluorotetrahydro- | | |
| | 2H-pyran-2-yl)pyrimidin-5- | | |
| | yl)-2-ethoxypyrimidine-5- | | |
| | carboxamide | | |
| | P2 | | |
| 534 | 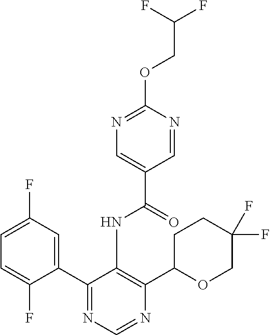 [SMILES: O=C(Nc1c(-c2cc(F)ccc2F)ncnc1C1CCC(F)(F)CO1)c1cnc(OCC(F)F)nc1] | DAICEL CHIRALCEL OJ (250 mm × 30 mm, 5 μm) eluting with 15% of methanol containing 0.1% ammonium hydroxide in supercritical carbon dioxide | 0.831 min, 99%; (400 MHz, CDCl₃) δ 9.18-9.13 (m, 1H), 8.95-8.85 (m, 3H), 7.47-7.41 (m, 1H), 7.13-7.00 (m, 2H), 6.34-6.00 (m, 1H), 4.86-4.80 (m, 1H), 4.70-4.63 (m, 2H), 4.22-4.07 (m, 1H), 3.79-3.69 (m, 1H), 2.40-2.32 (m, 1H), 2.27-2.19 (m, 1H), 2.17-2.04 (m, 2H); MS (ES+) m/z 514.2 (M + 1) |
| | 2-(2,2-difluoroethoxy)-N- | | |
| | (4-(2,5-difluorophenyl)-6- | | |
| | (5,5-difluorotetrahydro-2H- | | |
| | pyran-2-yl)pyrimidin-5- | | |
| | yl)pyrimidine-5- | | |
| | carboxamide | | |
| | P1 | | |
| 535 | 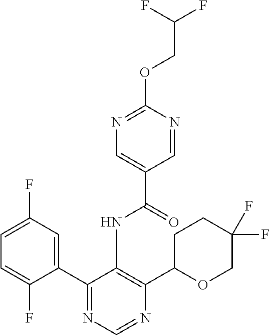 [SMILES: O=C(Nc1c(-c2cc(F)ccc2F)ncnc1C1CCC(F)(F)CO1)c1cnc(OCC(F)F)nc1] | 15% of methanol containing 0.1% ammonium hydroxide in supercritical carbon dioxide | 0.962 min, 99%; (400 MHz, CDCl₃) δ 9.17-9.14 (m, 1H), 8.96-8.92 (m, 1H), 8.90-8.87 (m, 2H), 7.48-7.40 (m, 1H), 7.13-6.99 (m, 2H), 6.32-6.01 (m, 1H), 4.87-4.79 (m, 1H), 4.70-4.63 (m, 2H), 4.23-4.10 (m, 1H), 3.79-3.69 (m, 1H), 2.41-2.31 (m, 1H), 2.27-2.19 (m, 1H), 2.17-2.04 (m, 2H); MS (ES+) m/z 514.2 (M + 1) |
| | 2-(2,2-difluoroethoxy)-N- | | |
| | (4-(2,5-difluorophenyl)-6- | | |
| | (5,5-difluorotetrahydro-2H- | | |
| | pyran-2-yl)pyrimidin-5- | | |
| | yl)pyrimidine-5- | | |
| | carboxamide | | |
| | P2 | | |

### Example 536

### Synthesis of N-(4-(4,4-difluorocyclohexyl)-6-(3-fluoropyridin-2-yl)pyrimidin-5-yl)-2-isopropylpyrimidine-5-carboxamide

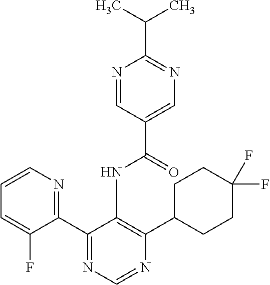 [SMILES: CC(C)c1ncc(C(=O)Nc2c(-c3ncccc3F)ncnc2C2CCC(F)(F)CC2)cn1]

### Step 1. Preparation of 2-(I³-methyl)propan-2-yl (4-chloro-6-(3-fluoropyridin-2-yl)pyrimidin-5-yl)carbamate

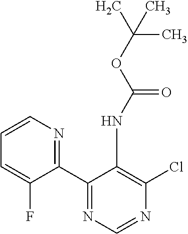 [SMILES: CC(C)(C)OC(=O)Nc1c(Cl)ncnc1-c1ncccc1F]

To a solution of tert-butyl N-(4,6-dichloropyrimidin-5-yl)carbamate (0.50 g, 1.9 mmol) in N,N-dimethylformamide (7.6 mL) were added tetrakis(triphenylphosphine)palladium(0) (0.13 g, 0.11 mmol), copper (1) iodide (0.036 g, 0.19 mmol) and 3-fluoro-2-(tributylstannyl)pyridine (0.73 g, 1.9 mmol). After stirring the mixture under a nitrogen atmosphere at 100° C. for 3 h, the mixture was cooled to ambient temperature and diluted with ethyl acetate (20 mL) was washed with ammonium chloride solution (2×20 mL), 1M potassium fluoride solution (20 mL), and brine (20 mL), and then concentrated in vacuo. The residue was purified by column chromatography, eluting with a gradient of 5 to 95% ethyl acetate in heptane, to afford the title compound as a yellow solid (0.32 g, 52% yield): MS (ES+) m/z 325.2 (M+1), 327.2 (M+1).

### Step 3. Preparation of tert-butyl (4-(4,4-difluorocyclohexyl)-6-(3-fluoropyridin-2-yl)pyrimidin-5-yl)carbamate

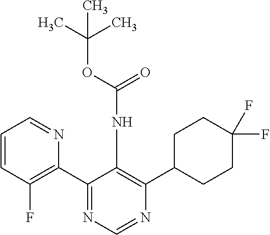 [SMILES: CC(C)(C)OC(=O)Nc1c(-c2ncccc2F)ncnc1C1CCC(F)(F)CC1]

To a vial containing 2-(I³-methyl)propan-2-yl (4-chloro-6-(3-fluoropyridin-2-yl)pyrimidin-5-yl)carbamate (0.30 g, 0.92 mmol) was added 4,4-Difluorocyclohexanecarboxylic acid (0.23 g, 1.4 mmol), (4,4″-di-t-butyl-2,2″-bipyridine)bis[3,5-difluoro-2-[5-trifluoromethyl-2-pyridinyl-kN)phenyl-kC]iridium(II) hexafluorophosphate (0.010 g, 0.0092 mmol), dichloro(dimethoxyethane)nickel (0.020 g, 0.092 mmol), 4-tert-butyl-2-(4-tert-butyl-2-pyridyl)pyridine (0.037 g, 0.14 mmol), cesium carbonate (0.54 g, 1.7 mmol), and N,N-dimethylformamide (18 mL). The vial was sealed, and the reaction mixture was stirred in front of Kessil PR160L lights (440 nm) for 20 h. The reaction mixture was diluted with ethyl acetate (250 mL) and washed with saturated ammonium chloride solution (2×50 mL), water (50 mL), and brine (50 mL). The organic layer was dried over anhydrous magnesium sulfate, filtered, and concentrated in vacuo. The resulting residue was used as is in the next step without further purification (0.27 g, 72% yield): MS (ES+) m/z 409.4 (M+1).

### Step 4. Preparation of 4-(4,4-difluorocyclohexyl)-6-(3-fluoropyridin-2-yl)pyrimidin-5-amine

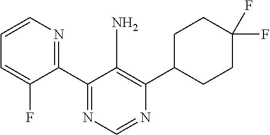 [SMILES: Nc1c(-c2ncccc2F)ncnc1C1CCC(F)(F)CC1]

To a mixture of tert-butyl (4-(4,4-difluorocyclohexyl)-6-(3-fluoropyridin-2-yl)pyrimidin-5-yl)carbamate (0.27 g, 0.66 mmol) in 1,4-dioxane (1.3 mL) was added 4 M hydrogen chloride in 1,4-dioxane (1.7 mL, 6.6 mmol). The reaction mixture was stirred at ambient temperature for 20 h. The reaction mixture was diluted with ethyl acetate (20 mL) and washed with saturated ammonium hydroxide solution (2×20 mL), and brine (20 mL). The organic layer was dried over anhydrous magnesium sulfate, filtered, and concentrated in vacuo. Purification of the residue by column chromatography, eluting with 5-50% ethyl acetate in heptane, afforded the title compound as a light-yellow solid (0.050 g, 25% yield): MS (ES+) m/z 309.3 (M+1).

### Step 5. Preparation of N-(4-(4,4-difluorocyclohexyl)-6-(3-fluoropyridin-2-yl)pyrimidin-5-yl)-2-isopropylpyrimidine-5-carboxamide

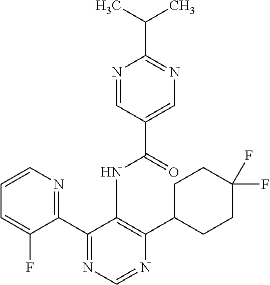 [SMILES: CC(C)c1ncc(C(=O)Nc2c(-c3ncccc3F)ncnc2C2CCC(F)(F)CC2)cn1]

To a mixture of 4-(4,4-difluorocyclohexyl)-6-(3-fluoropyridin-2-yl)pyrimidin-5-amine (0.026 g, 0.084 mmol), 2-isopropylpyrimidine-5-carboxylic acid (0.021 g, 0.13 mmol) and 2-chloro-1-methylpyridinium iodide (0.065 g, 0.25 mmol) and N,N-diisopropylethylamine (0.088 mL, 0.51 mmol) added anhydrous tetrahydrofuran (0.85 mL). The reaction mixture was stirred at 68° C. for 3 h. After cooling to ambient temperature, the mixture was diluted with methanol (2 mL) and 1M sodium hydroxide (1.5 mL). The mixture was stirred at ambient temperature for 30 min before it was diluted with ethyl acetate (20 mL). The organic layer was washed with saturated ammonium chloride solution (2×30 mL), dried over anhydrous magnesium sulfate, filtered, and concentrated in vacuo. Purification of the residue by column chromatography, eluting with a gradient of 5 to 80% ethyl acetate in heptane, afforded the title compound as a colorless solid (0.0091 g, 23% yield): ¹H-NMR (400 MHz; DMSO-d₆) δ 9.22 (s, 1H), 9.02 (s, 2H), 8.48 (dt, J=4.6, 1.3 Hz, 1H), 7.77 (ddd, J=9.9, 8.6, 1.2 Hz, 1H), 7.56 (dt, J=8.6, 4.3 Hz, 1H), 3.31-3.23 (m, 2H), 2.22-2.15 (m, 2H), 2.14-2.05 (m, 2H), 2.00-1.96 (m, 3H), 1.92-1.84 (m, 1H), 1.36 (d, J=6.9 Hz, 6H); MS (ES+) m/z 457.3 (M+1).

In a similar manner as described in examples disclosed herein, utilizing the appropriately substituted starting materials and intermediates, the following compounds were prepared:

| Example | | Amount | | |
|---|---|---|---|---|
| No. | Structure | Yield | MS (ES+) m/z | ¹H NMR |
| 537 | 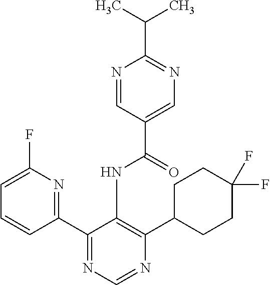 [SMILES: CC(C)c1ncc(C(=O)Nc2c(-c3cccc(F)n3)ncnc2C2CCC(F)(F)CC2)cn1] | 0.0065 g 29% | 457.2 (M + 1) | (400 MHz, DMSO-d₆) δ 10.68 (s, 1H), 9.24 (s, 1H), 9.16 (s, 2H), 8.18 (q, J = 8.0 Hz, 1H), 8.07 (dd, J = 7.4, 2.1 Hz, 1H), 7.30 (dd, J = 8.2, 2.6 Hz, 1H), 3.31- 3.30 (m, 1H), 3.24 (dq, J = 13.8, 6.9 Hz, 1H), 2.16-2.02 (m, 3H), 1.96-1.88 (m, 5H), 1.32 (d, J = 6.9 Hz, 6H) |
| | N-(4-(4,4- | | | |
| | difluorocyclohexyl)-6-(6- | | | |
| | fluoropyridin-2- | | | |
| | yl)pyrimidin-5-yl)-2- | | | |
| | isopropylpyrimidine-5- | | | |
| | carboxamide | | | |

### Example 538

### Synthesis of N-(4-(4,4-difluorocyclohexyl)-6-(pyridin-2-yl)pyrimidin-5-yl)-5-fluoro-6-methoxynicotinamide

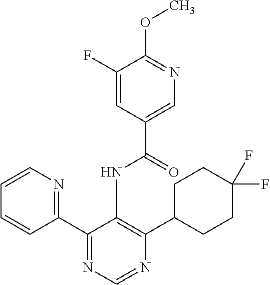 [SMILES: COc1ncc(C(=O)Nc2c(-c3ccccn3)ncnc2C2CCC(F)(F)CC2)cc1F]

### Step 1. Preparation of 4-chloro-6-(3-fluoropyridin-2-yl)pyrimidin-5-amine

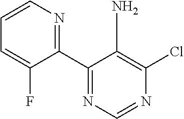 [SMILES: Nc1c(Cl)ncnc1-c1ncccc1F]

To a solution of 5-amino-4,6-dichloropyrimidine (0.50 g, 3.0 mmol) in N,N-dimethylformamide (24 mL) were added tetrakis(triphenylphosphine)palladium(0) (0.2 g, 0.18 mmol), copper (1) iodide (0.058 g, 0.30 mmol) and 3-fluoro-2-(tributylstannyl)pyridine (0.82 g, 2.1 mmol). After stirring the mixture under nitrogen at 100° C. for 3 h, the mixture was cooled to ambient temperature and diluted with ethyl acetate (15 mL) was washed with 1M hydrochloric acid solution (2×20 mL) and brine (20 mL), and concentrated in vacuo. The residue was purified by column chromatography, eluting with a gradient of 5 to 95% ethyl acetate in heptane, to afford the title compound as a yellow solid (0.19 g, 27% yield): MS (ES+) m/z 225.2 (M+1), 227.2 (M+1).

### Step 2. Preparation of 4-(4,4-difluorocyclohex-1-en-1-yl)-6-(3-fluoropyridin-2-yl)pyrimidin-5-amine

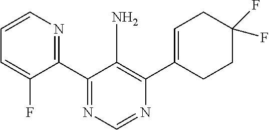 [SMILES: Nc1c(C2=CCC(F)(F)CC2)ncnc1-c1ncccc1F]

To 4-chloro-6-(3-fluoropyridin-2-yl)pyrimidin-5-amine (0.17 g, 0.77 mmol) was added 1,4-dioxane (4.6 mL) and water (0.51 mL) and the mixture was sparged with nitrogen for 10 min. To the mixture was added 2-(4,4-difluorocyclohex-1-en-1-yl)-4,4,5,5-tetramethyl-1,3,2-dioxaborolane (0.28 g, 1.2 mmol), potassium carbonate (0.32 g, 2.3 mmol), and [1,1′-bis(diphenylphosphino)ferrocene]dichloropalladium(II), complex with dichloromethane (0.063 g, 0.077 mmol), and the solution was sparged with nitrogen for 2 min. The flask was sealed under a nitrogen atmosphere and heated to 90° C. for 4 h. The reaction mixture was cooled to ambient temperature and diluted with ethyl acetate (100 mL). The organic layer was washed with saturated ammonium chloride (2×50 mL), water (100 mL), dried over anhydrous magnesium sulfate, filtered, and concentrated in vacuo. Purification of the residue by column chromatography, eluting with 5 to 95% ethyl acetate in heptane, afforded the title compound as a brown solid (0.16 g, 66% yield): MS (ES+) m/z 307.4 (M+1).

### Step 3. Preparation of 4-(4,4-difluorocyclohexyl)-6-(2,5-difluorophenyl)pyrimidin-5-amine

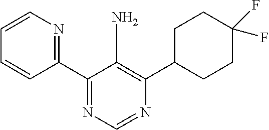 [SMILES: Nc1c(-c2ccccn2)ncnc1C1CCC(F)(F)CC1]

A mixture of 4-(4,4-difluorocyclohex-1-en-1-yl)-6-(2,5-difluorophenyl)pyrimidin-5-amine (0.16 g, 0.51 mmol) in methanol (2.5 mL) and ethyl acetate (2.5 mL) was degassed with nitrogen for 10 minutes. To the reaction mixture was added 10% palladium on carbon (0.54 g), ammonium formate (0.64 g, 10 mmol) and acetic acid (0.087 mL, 1.5 mmol). The reaction was stirred at 65° C. for 1 hour and left to stir at 45° C. for 24 h. The mixture was diluted with ethyl acetate (30 mL), filtered through a bed of Celite and the filtrate was concentrated in vacuo. Purification by column chromatography using a gradient of 5-50% ethyl acetate in heptane, and then 0 to 25% methanol in ethyl acetate, afforded the title compound as an off-white solid (0.11 g, 69% yield): ¹H-NMR (400 MHz; DMSO-d₆) δ 8.72 (dt, J=4.9, 0.8 Hz, 1H), 8.56 (d, J=8.1 Hz, 1H), 8.48 (s, 1H), 8.01 (td, J=7.8, 1.6 Hz, 1H), 7.50-7.47 (m, 1H), 7.45 (d, J=10.1 Hz, 2H), 3.21-3.15 (m, 1H), 2.20-2.13 (m, 2H), 2.10-1.91 (m, 4H), 1.84-1.74 (m, 2H); MS (ES+) m/z 291.2 (M+1).

### Step 4. Preparation of N-(4-(4,4-difluorocyclohexyl)-6-(pyridin-2-yl)pyrimidin-5-yl)-5-fluoro-6-methoxynicotinamide

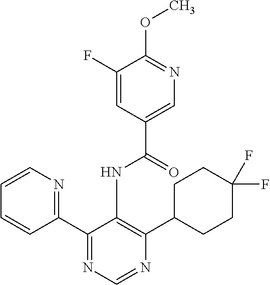 [SMILES: COc1ncc(C(=O)Nc2c(-c3ccccn3)ncnc2C2CCC(F)(F)CC2)cc1F]

To a mixture of 4-(4,4-difluorocyclohexyl)-6-(2,5-difluorophenyl)pyrimidin-5-amine (0.10 g, 0.35 mmol), 5-fluoro-6-methoxynicotinic acid (0.091 g, 0.53 mmol) and 2-chloro-1-methylpyridinium iodide (0.27 g, 1.1 mmol) and N,N-diisopropylethylamine (0.37 mL, 2.1 mmol) added anhydrous tetrahydrofuran (3.4 mL). The reaction mixture was stirred at 68° C. for 3 h. After cooling to ambient temperature, the mixture was diluted with methanol (5 mL) and 1M sodium hydroxide (2 mL). The mixture was stirred at ambient temperature for 30 min before it was diluted with ethyl acetate (20 mL). The organic layer was washed with saturated ammonium chloride solution (2×30 mL), dried over anhydrous magnesium sulfate, filtered and concentrated in vacuo. Purification of the residue by column chromatography, eluting with a gradient of 5 to 80% ethyl acetate in heptane, afforded the title compound a colorless solid (0.12 g, 71% yield): ¹H-NMR (400 MHz; DMSO-d₆) δ 10.69 (s, 1H), 9.20 (s, 1H), 8.57-8.55 (m, 2H), 8.10-8.06 (m, 2H), 7.99 (td, J=7.8, 1.7 Hz, 1H), 7.48 (ddd, J=7.5, 4.8, 0.9 Hz, 1H), 4.04 (s, 3H), 3.24-3.19 (m, 1H), 2.15-2.11 (m, 2H), 2.02-1.84 (m, 6H); MS (ES+) m/z 444.2 (M+1).

### Example 539 and 540

### Synthesis of N-(4-(2,5-difluorophenyl)-6-(syn-2-(trifluoromethyl)cyclohexyl)pyrimidin-5-yl)-2-isopropylpyrimidine-5-carboxamide P1,D1/P2,D1

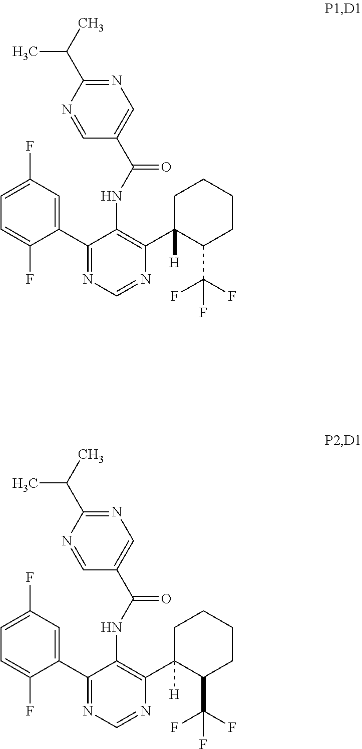 [SMILES: CC(C)c1ncc(C(=O)Nc2c(-c3cc(F)ccc3F)ncnc2[C@@H]2CCCC[C@@H]2C(F)(F)F)cn1 ; CC(C)c1ncc(C(=O)Nc2c(-c3cc(F)ccc3F)ncnc2[C@H]2CCCC[C@H]2C(F)(F)F)cn1]

### Step 1. Preparation of tert-butyl (4-(2,5-difluorophenyl)-6-(6-(trifluoromethyl)cyclohex-1-en-1-yl)pyrimidin-5-yl)carbamate

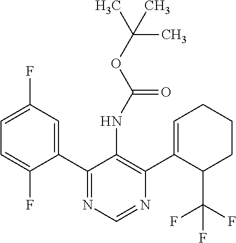 [SMILES: CC(C)(C)OC(=O)Nc1c(C2=CCCCC2C(F)(F)F)ncnc1-c1cc(F)ccc1F]

A mixture of tert-butyl N-[4-chloro-6-(2,5-difluorophenyl)pyrimidin-5-yl]carbamate (0.25 g, 0.72 mmol) in 1,4-dioxane (4.5 mL) was degassed with nitrogen for 10 minutes. To the reaction mixture was added 4-methyl-N—[(Z)-[2-(trifluoromethyl)cyclohexylidene]amino]benzenesulfonamide (0.36 g, 1.1 mmol), tris(dibenzylideneacetone)dipalladium(0) (0.066 g, 0.072 mmol), tricyclohexylphosphine tetrafluoroborate (0.053 g, 0.14 mmol) and lithium tert-butoxide (0.17 g, 2.2 mmol). The reaction was stirred at 110° C. for 16 h. After cooling to ambient temperature, the reaction mixture was diluted in water (30 mL) and the aqueous phase was extracted with ethyl acetate (3×20 mL). The combined organic phase was washed with brine (20 mL), dried over anhydrous magnesium sulfate, filtered, and concentrated in vacuo. The residue was purified by column chromatography, eluting with a gradient of 5 to 60% ethyl acetate in heptane, to afford the title compound as an orange solid (0.14 g, 42% yield): MS (ES+) m/z 456.4 (M+1).

### Step 2. Preparation of 4-(2,5-difluorophenyl)-6-(6-(trifluoromethyl)cyclohex-1-en-1-yl)pyrimidin-5-amine

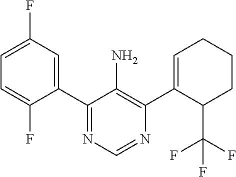 [SMILES: Nc1c(C2=CCCCC2C(F)(F)F)ncnc1-c1cc(F)ccc1F]

To tert-butyl (4-(2,5-difluorophenyl)-6-(2-(trifluoromethyl)cyclohexyl)pyrimidin-5-yl)carbamate (0.14 g, 0.30 mmol) was added 4.0 M hydrochloric acid in anhydrous dioxane (1.5 mL, 6.1 mmol) and 1,4-dioxane (1.2 mL), and the reaction mixture was stirred at ambient temperature for 16 h. The reaction mixture was diluted in saturated potassium carbonate (15 mL) and the aqueous phase was extracted with ethyl acetate (3×20 mL). The combined organic phase was washed with brine (20 mL), dried over anhydrous magnesium sulfate, filtered, and concentrated in vacuo. The resulting residue was used as is in the next step (0.11 g, quant. yield): MS (ES+) m/z 356.2 (M+1).

### Step 3. Preparation of 4-(2,5-difluorophenyl)-6-(2-(trifluoromethyl)cyclohexyl)pyrimidin-5-amine

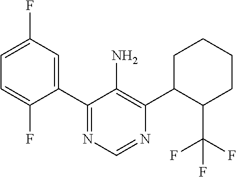 [SMILES: Nc1c(-c2cc(F)ccc2F)ncnc1C1CCCCC1C(F)(F)F]

A mixture of 4-(2,5-difluorophenyl)-6-(6-(trifluoromethyl)cyclohex-1-en-1-yl)pyrimidin-5-amine (0.11 g, 0.30 mmol) in ethanol (5.0 mL) and acetic acid (2.0 mL) was degassed with nitrogen for 10 minutes. To the reaction mixture was added ammonium formate (0.19 g, 3.0 mmol) and 10% palladium hydroxide (0.047 g, 0.33 mmol). The reaction mixture was stirred at 80° C. for 2.5 h. After cooling to ambient temperature, the mixture was diluted in ethyl acetate (25 mL) and filtered through a bed of Celite. The filtrate was washed with sodium bicarbonate (15 mL) and brine (15 mL), dried with anhydrous magnesium sulfate, and concentrated in vacuo. The resulting residue was used as is in the next step (0.11 g, 99% yield): MS (ES+) m/z 358.4 (M+1).

### Step 4. Preparation of rac-N-(4-(2,5-difluorophenyl)-6-(2-(trifluoromethyl)cyclohexyl)pyrimidin-5-yl)-2-isopropylpyrimidine-5-carboxamide

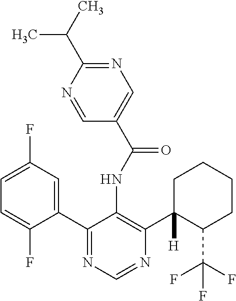 [SMILES: CC(C)c1ncc(C(=O)Nc2c(-c3cc(F)ccc3F)ncnc2[C@@H]2CCCC[C@@H]2C(F)(F)F)cn1]

To a mixture of 4-(2,5-difluorophenyl)-6-(2-(trifluoromethyl)cyclohexyl)pyrimidin-5-amine (0.11 g, 0.30 mmol) in anhydrous tetrahydrofuran (3.0 mL) was added N,N-diisopropylethylamine (0.31 mL, 1.8 mmol), 2-chloro-1-methylpyridinium iodide (0.23 g, 0.89 mmol) and 2-isopropylpyrimidine-5-carboxylic acid (0.074 g, 0.45 mmol). The reaction mixture was stirred at 65° C. for 8 h. After cooling to ambient temperature, to the reaction mixture was added methanol (3 mL) and 10 M sodium hydroxide (1 mL) and the mixture was stirred at ambient temperature for 10 minutes. The reaction mixture was diluted with ethyl acetate (10 mL), and the organic phase was washed with 1 M sodium hydroxide (20 mL) and saturated ammonium chloride (20 mL), dried with anhydrous magnesium sulfate, filtered, and concentrated in vacuo. The residue was purified by column chromatography, eluting with a gradient of 5 to 90% ethyl acetate in heptane. Further purification by reverse-phase column chromatography, using a gradient of 10 to 95% acetonitrile in water as eluent, afforded a single diastereomer as colorless solid (0.064 g, 41% yield): ¹H NMR (400 MHz, DMSO-d₆) δ 10.63 (d, J=0.5 Hz, 1H), 9.23 (s, 1H), 8.98 (s, 2H), 7.41-7.32 (m, 3H), 3.78-3.68 (m, 1H), 3.20 (7, J=6.9 Hz, 1H), 2.91-2.81 (m, 1H), 2.47-2.40 (m, 1H), 2.09-1.92 (m, 2H), 1.84-1.67 (m, 3H), 1.52-1.39 (m, 2H), 1.29 (d, J=6.9 Hz, 6H); MS (ES+) m/z 506.3 (M+1).

### Step 5. Preparation of N-(4-(2,5-difluorophenyl)-6-(2-(trifluoromethyl)cyclohexyl)pyrimidin-5-yl)-2-isopropylpyrimidine-5-carboxamide P1,D1/P2,D1

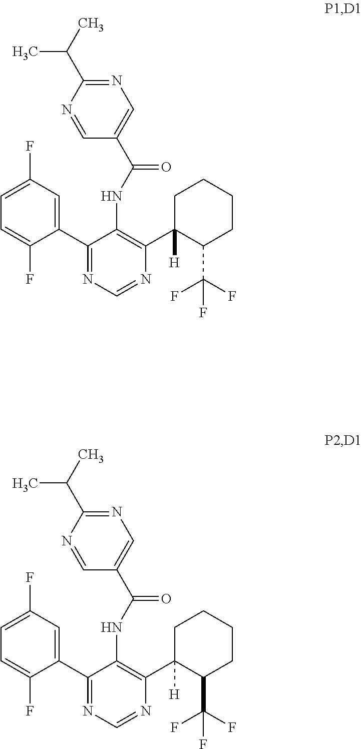 [SMILES: CC(C)c1ncc(C(=O)Nc2c(-c3cc(F)ccc3F)ncnc2[C@@H]2CCCC[C@@H]2C(F)(F)F)cn1 ; CC(C)c1ncc(C(=O)Nc2c(-c3cc(F)ccc3F)ncnc2[C@H]2CCCC[C@H]2C(F)(F)F)cn1]

N-(4-(2,5-difluorophenyl)-6-(2-(trifluoromethyl)cyclohexyl)pyrimidin-5-yl)-2-isopropylpyrimidine-5-carboxamide (0.050 g, 0.099 mmol) was purified by chiral SFC (column: DAICEL CHIRALPAK AD (250 mm×30 mm, 10 μm), eluting with 25% of ethanol containing 0.1% ammonium hydroxide in supercritical carbon dioxide, to afford peak 1 (retention time=0.779 min) as a colorless solid (0.025 g, 24% yield, 99% ee): ¹H NMR (400 MHz, DMSO-d₆) δ 10.59 (s, 1H), 9.23 (s, 1H), 8.97 (s, 2H), 7.40-7.32 (m, 3H), 3.81-3.67 (m, 1H), 3.20 (td, J=6.8, 13.6 Hz, 1H), 2.94-2.76 (m, 1H), 2.10-1.64 (m, 6H), 1.52-1.41 (m, 2H), 1.29 (d, J=6.8 Hz, 6H); MS (ES+) m/z 506.3 (M+1).

Peak 2 (retention time=1.118 min) afforded a colorless solid (0.030 g, 29% yield, 95% ee): ¹H NMR (400 MHz, DMSO-d₆) δ 10.62-10.57 (m, 1H), 9.23 (s, 1H), 8.97 (s, 2H), 7.35 (dd, J=5.6, 8.0 Hz, 3H), 3.79-3.67 (m, 1H), 3.24-3.16 (m, 1H), 2.93-2.77 (m, 1H), 2.15-1.61 (m, 6H), 1.52-1.39 (m, 2H), 1.29 (d, J=6.8 Hz, 6H); MS (ES+) m/z 506.3 (M+1).

In a similar manner as described in examples disclosed herein, utilizing the appropriately substituted starting materials and intermediates, the following compounds were prepared:

| Example | Structure | | | |
|---|---|---|---|---|
| No. | Name | Amount | MS (ES+) m/z | ¹H NMR |
| 541 | 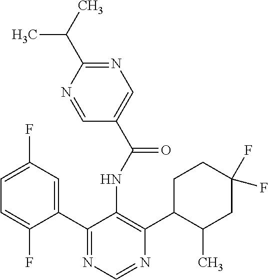 [SMILES: CC(C)c1ncc(C(=O)Nc2c(-c3cc(F)ccc3F)ncnc2C2CCC(F)(F)CC2C)cn1] | 0.0070 g 6.1% | 488.4 (M + 1) | (400 MHz, DMSO-d₆) δ 10.71 (s, 1H), 9.24 (s, 1H), 9.00 (s, 2H), 7.41- 7.36 (m, 3H), 3.22 (dq, J = 13.8, 6.9 Hz, 1H), 2.96-2.89 (m, 1H), 2.29-2.19 (m, 1H), 2.14-2.06 (m, 2H), 1.97-1.66 (m, 4H), 1.29 (d, J = 6.9 Hz, 6H), 0.66 (d, J = 6.2 Hz, 3H) |
| | N-(4-(4,4-difluoro-2- | | | |
| | methylcyclohexyl)-6- | | | |
| | (2,5- | | | |
| | difluorophenyl)pyrimidin- | | | |
| | 5-yl)-2- | | | |
| | isopropylpyrimidine-5- | | | |
| | carboxamide | | | |
| | P1 | | | |
| 542 | 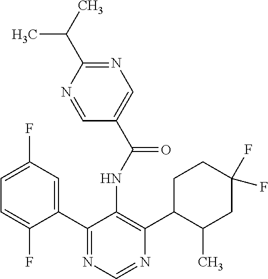 [SMILES: CC(C)c1ncc(C(=O)Nc2c(-c3cc(F)ccc3F)ncnc2C2CCC(F)(F)CC2C)cn1] | 0.015 g 13% | 488.3 (M + 1) | (400 MHz, DMSO-d₆) δ 10.66 (s, 1H), 9.23 (s, 1H), 8.98 (s, 2H), 7.41- 7.32 (m, 3H), 3.52 (s, 1H), 3.20 (dquintet, J = 13.8, 6.9 Hz, 1H), 2.36- 1.83 (m, 7H), 1.29 (d, J = 6.9 Hz, 6H), 0.76 (d, J = 6.7 Hz, 3H) |
| | N-(4-(4,4-difluoro-2- | | | |
| | methylcyclohexyl)-6- | | | |
| | (2,5- | | | |
| | difluorophenyl)pyrimidin- | | | |
| | 5-yl)-2- | | | |
| | isopropylpyrimidine-5- | | | |
| | carboxamide | | | |
| | P2 | | | |
| 543 | 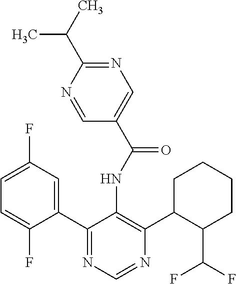 [SMILES: CC(C)c1ncc(C(=O)Nc2c(-c3cc(F)ccc3F)ncnc2C2CCCCC2C(F)F)cn1] | 0.034 g 30% | 488.3 (M + 1) | (400 MHz, DMSO-d₆) δ 10.63 (s, 1H), 9.21 (s, 1H), 8.98 (s, 2H), 7.41- 7.32 (m, 3H), 6.07 (td, J = 56.6, 5.6 Hz, 1H), 3.62 (s, 1H), 3.20 (dquintet, J = 13.8, 6.9 Hz, 1H), 2.40-2.23 (m, 2H), 2.08-2.01 (m, 1H), 1.71-1.64 (m, 3H), 1.60-1.55 (m, 1H), 1.45-1.39 (m, 2H), 1.29 (d, J = 6.9 Hz, 6H) |
| | N-(4-(2- | | | |
| | (difluoromethyl)cyclohexyl)- | | | |
| | 6-(2,5- | | | |
| | difluorophenyl)pyrimidin- | | | |
| | 5-yl)-2- | | | |
| | isopropylpyrimidine-5- | | | |
| | carboxamide | | | |

### Example 544

In a similar manner as described in examples described herein, utilizing the appropriately substituted starting materials and intermediates, the following compounds were prepared:

| Example | | Amount | MS (ES+) | |
|---|---|---|---|---|
| No. | Structure | Yield | m/z | ¹H NMR |
| 544 | 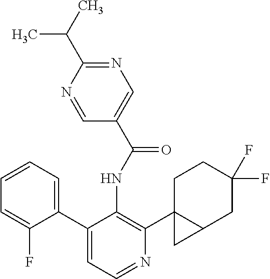 [SMILES: CC(C)c1ncc(C(=O)Nc2c(-c3ccccc3F)ccnc2C23CCC(F)(F)CC2C3)cn1] | 0.030 g 11% | 467.2 (M + 1) | (400 MHz, DMSO-d₆) δ 10.13 (s, 1H), 8.91 (s, 2H), 8.55 (d, J = 4.9 Hz, 1H), 7.41-7.38 (m, 3H), 7.29 (t, J = 9.4 Hz, 1H), 7.21 (t, J = 7.0 Hz, 1H), 3.18 (sept, J = 6.9 Hz, 1H), 2.37-2.29 (m, 1H), 2.24-2.11 (m, 2H), 2.09-1.95 (m, 1H), 1.84-1.73 (m, 2H), 1.55-1.48 (m, 1H), 1.27 (d, J = 6.9 Hz, 6H), 1.23-1.19 (m, 1H), 0.89 (t, J = 5.6 Hz, 1H) |
| | N-(2-(4,4 | | | |
| | difluorobicyclo[4.1.0] | | | |
| | heptan-1-yl)-4-(2- | | | |
| | fluorophenyl)pyridin-3-yl)- | | | |
| | 2-isopropylpyrimidine-5- | | | |
| | carboxamode | | | |

### Example 545-568

In a similar manner as described in examples described herein, utilizing the appropriately substituted starting materials and intermediates, the following compounds were prepared:

| Example | Structure | Amount | MS (ES+) | |
|---|---|---|---|---|
| No. | Name | Yield | m/z | ¹H NMR; ¹⁹F NMR |
| 545 | 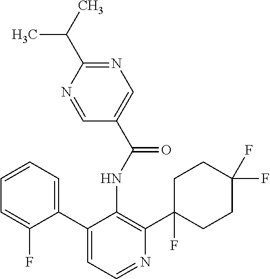 [SMILES: CC(C)c1ncc(C(=O)Nc2c(-c3ccccc3F)ccnc2C2(F)CCC(F)(F)CC2)cn1] | 0.023 g 8% | 473.6 (M + 1) | (400 MHz, DMSO-d₆) δ 10.28 (s, 1H), 8.86 (s, 2H), 8.65 (d, J = 4.9 Hz, 1H), 7.53 (d, J = 4.7 Hz, 1H), 7.44-7.38 (m, 2H), 7.30 (t, J = 9.2 Hz, 1H), 7.24-7.20 (m, 1H), 3.17 (sept, J = 6.9 Hz, 1H), 2.33- 2.07 (m, 8H), 1.26 (d, J = 6.9 Hz, 6H); (376 MHz, DMSO- d₆) δ −90.9, −102.5, −114.9, −165.9 |
| | N-(4-(2-fluorophenyl)-2- | | | |
| | (1,4,4- | | | |
| | trifluorocyclohexyl)pyridin- | | | |
| | 3-yl)-2- | | | |
| | isopropylpyrimidine-5- | | | |
| | carboxamide | | | |
| 546 | 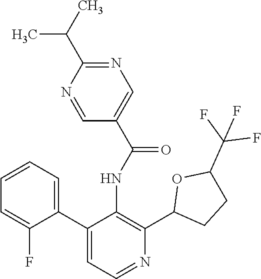 [SMILES: CC(C)c1ncc(C(=O)Nc2c(-c3ccccc3F)ccnc2C2CCC(C(F)(F)F)O2)cn1] | 0.069 g 28% | 475.6 (M + 1) | (400 MHz, DMSO-d₆) δ 10.45 (s, 1H), 8.93 (s, 2H), 8.67 (d, J = 4.8 Hz, 1H), 7.48 (d, J = 4.7 Hz, 1H), 7.46-7.36 (m, 2H), 7.30 (t, J = 9.2 Hz, 1H), 7.26-7.22 (m, 1H), 5.50 (t, J = 6.4 Hz, 1H), 4.80-4.71 (m, 1H), 3.18 (sept, J = 6.9 Hz, 1H), 2.49-2.40 (m, 1H), 2.37-2.21 (m, 2H), 2.11-2.02 (m, 1H), 1.27 (d, J = 6.9 Hz, 6H); (376 MHz, DMSO-d₆) δ −77.0, −115.0 |
| | N-(4-(2-fluorophenyl)-2-(5- | | | |
| | (trifluoromethyl)tetrahydro- | | | |
| | furan-2-yl)pyridin-3-yl)-2- | | | |
| | isopropylpyrimidine-5- | | | |
| | carboxamide | | | |
| | D1 | | | |
| 547 | 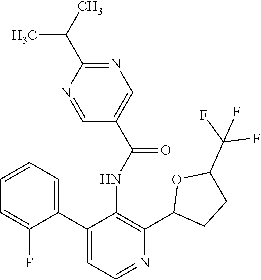 [SMILES: CC(C)c1ncc(C(=O)Nc2c(-c3ccccc3F)ccnc2C2CCC(C(F)(F)F)O2)cn1] | 0.007 g 3% | 475.6 (M + 1) | (400 MHz, DMSO-d₆) δ 10.46 (s, 1H), 8.94 (s, 2H), 8.69 (d, J = 4.8 Hz, 1H), 7.49-7.48 (m, 1H), 7.45-7.36 (m, 3H), 7.32-7.22 (m, 2H), 5.34 (t, J = 6.4 Hz, 1H), 4.49 (dq, J = 13.2, 6.7 Hz, 1H), 3.18 (sept, J = 6.9 Hz, 1H), 2.46-2.42 (m, 1H), 2.36-2.27 (m, 1H), 2.25-2.19 (m, 2H), 1.27 (d, J = 6.9 Hz, 6H); (376 MHz, DMSO-d₆) δ −76.4, −115.0 |
| | N-(4-(2-fluorophenyl)-2-(5- | | | |
| | (trifluoromethyl)tetrahydro- | | | |
| | furan-2-yl)pyridin-3-yl)-2- | | | |
| | isopropylpyrimidine-5- | | | |
| | carboxamide | | | |
| | D2 | | | |
| 548 | 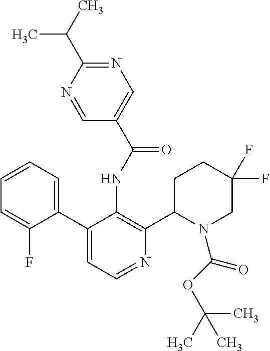 [SMILES: CC(C)c1ncc(C(=O)Nc2c(-c3ccccc3F)ccnc2C2CCC(F)(F)CN2C(=O)OC(C)(C)C)cn1] | 0.418 g 96% | 556.2 (M + 1) | (400 MHz, DMSO-d₆) δ 10.38-10.24 (m, 1H), 8.90 (s, 2H), 8.63 (d, J = 4.9 Hz, 1H), 7.50-7.49 (m, 1H), 7.45-7.36 (m, 2H), 7.30 (t, J = 9.2 Hz, 1H), 7.25-7.21 (m, 1H), 5.78- 5.48 (m, 1H), 4.21-4.01 (m, 1H), 3.41-3.25 (m, 1H), 3.16 (sept, J = 6.9 Hz, 1H), 2.70- 1.94 (m, 4H), 1.25 (d, J = 6.9 Hz, 6H), 1.18 (s, 9H) |
| | tert-butyl 5,5-difluoro-2-(4- | | | |
| | (2-fluorophenyl)-3-(2- | | | |
| | isopropylpyrimidine-5- | | | |
| | carboxamido)pyridin-2- | | | |
| | yl)piperidine-1-carboxylate | | | |
| 549 | 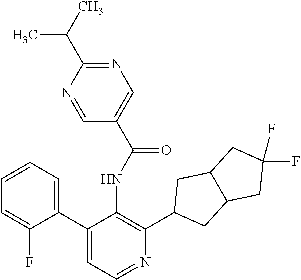 [SMILES: CC(C)c1ncc(C(=O)Nc2c(-c3ccccc3F)ccnc2C2CC3CC(F)(F)CC3C2)cn1] | 0.055 g 55% | 481.2 (M + 1) | (400 MHz, DMSO-d₆, dr = 1:2) δ 10.31 (s, 2H), 10.28 (s, 1H), 8.92 (s, 2H), 8.91 (d, J = 6.1 Hz, 4H), 8.61 (d, J = 4.9 Hz, 1H), 8.59 (d, J = 4.9 Hz, 2H), 7.44-7.20 (m, 15H), 3.78-3.69 (m, 2H), 3.45 (tt, J = 11.8, 6.0 Hz, 1H), 3.18 (sept, J = 6.9 Hz, 2H), 3.18 (sept, J = 6.9 Hz, 1H), 2.84- 2.75 (m, 4H), 2.67-2.60 (m, 2H), 2.33-1.90 (m, 15H), 1.82-1.69 (m, 9H), 1.27 (dd, J = 6.9, 1.7 Hz, 18H); (376 MHz, DMSO-d₆) δ −90.2 (d, J = 225 Hz), −92.3 (d, J = 225 Hz), −95.2 (d, J = 224 Hz), −96.4 (d, J = 224 Hz), −114.9, −115.0 |
| | N-(2-(5,5- | | | |
| | difluorooctahydropentalen- | | | |
| | 2-yl)-4-(2- | | | |
| | fluorophenyl)pyridin-3-yl)- | | | |
| | 2-isopropylpyrimidine-5- | | | |
| | carboxamide | | | |
| | dr = 1:2 | | | |
| 550 | 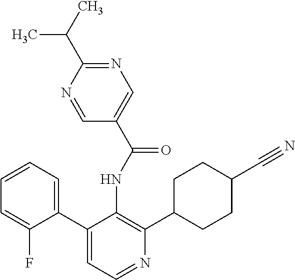 [SMILES: CC(C)c1ncc(C(=O)Nc2c(-c3ccccc3F)ccnc2C2CCC(C#N)CC2)cn1] | 0.165 g 69% | 444.2 (M + 1) | (400 MHz, DMSO-d₆, dr = 1:1) δ 10.31 (s, 2H), 8.92 (s, 4H), 8.65 (d, J = 4.9 Hz, 1H), 8.59 (d, J = 4.9 Hz, 1H), 7.44-7.21 (m, 10H), 3.24- 3.12 (m, 4H), 3.08-3.02 (m, 2H), 1.99-1.58 (m, 16H), 1.28 (dd, J = 6.9, 3.4 Hz, 12H); (376 MHz, DMSO-d₆) δ −114.9, −115 |
| | N-(2-(4-cyanocyclohexyl)- | | | |
| | 4-(2-fluorophenyl)pyridin- | | | |
| | 3-yl)-2- | | | |
| | isopropylpyrimidine-5- | | | |
| | carboxamide | | | |
| | dr = 1:1 | | | |
| 551 | 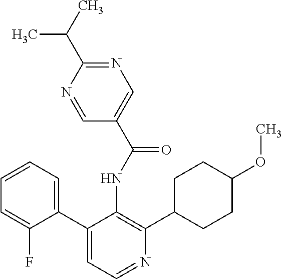 [SMILES: COC1CCC(c2nccc(-c3ccccc3F)c2NC(=O)c2cnc(C(C)C)nc2)CC1] | 0.031 g 13% | 449.2 (M + 1) | (400 MHz, DMSO-d₆) δ 10.29 (s, 1H), 8.92 (s, 2H), 8.59 (d, J = 4.9 Hz, 1H), 7.43-7.34 (m, 2H), 7.32-7.20 (m, 3H), 3.43 (s, 1H), 3.24 (s, 3H), 3.18 (sept, J = 6.9 Hz, 1H), 3.07-3.00 (m, 1H), 1.96-1.93 (m, 4H), 1.50-1.38 (m, 4H), 1.28 (d, J = 6.9 Hz, 6H); (376 MHz, DMSO-d₆) δ −114.9 |
| | N-(4-(2-fluorophenyl)-2-(4- | | | |
| | methoxycyclohexyl)pyridin- | | | |
| | 3-yl)-2- | | | |
| | isopropylpyrimidine-5- | | | |
| | carboxamide | | | |
| 552 | 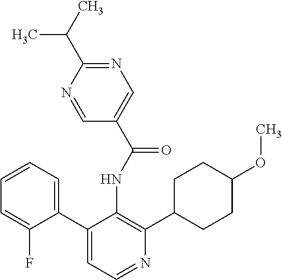 [SMILES: COC1CCC(c2nccc(-c3ccccc3F)c2NC(=O)c2cnc(C(C)C)nc2)CC1] | 0.050 g 21% | 449.2 (M + 1) | (400 MHz, DMSO-d₆) δ 10.30 (s, 1H), 8.91 (s, 2H), 8.58 (d, J = 4.9 Hz, 1H), 7.43-7.33 (m, 2H), 7.32-7.26 (m, 2H), 7.22 (td, J = 7.5, 1.0 Hz, 1H), 3.24-3.22 (m, 3H), 3.22-3.13 (m, 2H), 2.98 (tt, J = 11.5, 3.4 Hz, 1H), 2.10- 2.06 (m, 2H), 1.82-1.64 (m, 4H), 1.28 (d, J = 6.9 Hz, 6H), 1.24-1.13 (m, 2H); (376 MHz, DMSO-d₆) δ −115.0 |
| | N-(4-(2-fluorophenyl)-2-(4- | | | |
| | methoxycyclohexyl)pyridin- | | | |
| | δ-3-yl)-2- | | | |
| | isopropylpyrimidine-5- | | | |
| | carboxamide | | | |
| | D2 | | | |
| 553 | 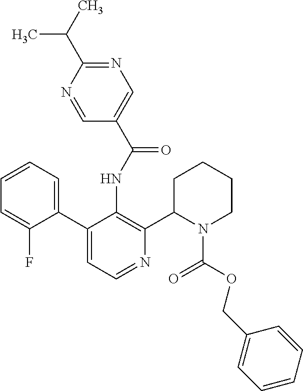 [SMILES: CC(C)c1ncc(C(=O)Nc2c(-c3ccccc3F)ccnc2C2CCCCN2C(=O)OCc2ccccc2)cn1] | 0.318 g 107% | 554.2 (M + 1) | (400 MHz, DMSO-d₆, rotamers are present) δ 10.36 (s, 1H), 8.89 (s, 2H), 8.58 (s, 1H), 7.41-7.18 (m, 10H), 5.66 (s, 1H), 4.91 (s, 2H), 3.93 (d, J = 10.4 Hz, 1H), 3.56-3.45 (m, 1H), 3.17 (sept, J = 6.9 Hz, 1H), 2.13- 2.07 (m, 1H), 1.81-1.66 (m, 3H), 1.56-1.51 (m, 1H), 1.46- 1.41 (m, 1H), 1.26 (d, J = 6.9 Hz, 6H) |
| | Benzyl 2-(4-(2- | | | |
| | fluorophenyl)-3-(2- | | | |
| | isopropylpyrimidine-5- | | | |
| | carboxamido)pyridin-2- | | | |
| | yl)piperidine-1-carboxylate | | | |
| 554 | 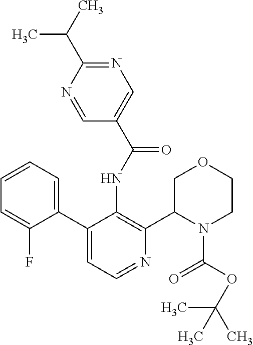 [SMILES: CC(C)c1ncc(C(=O)Nc2c(-c3ccccc3F)ccnc2C2COCCN2C(=O)OC(C)(C)C)cn1] | 0.249 g 88% | 522.3 (M + 1) | (400 MHz, DMSO-d₆) δ 10.44-10.17 (m, 0.3H), 8.88 (s, 2H), 8.58 (d, J = 4.9 Hz, 1H), 7.44-7.35 (m, 3H), 7.30 (t, J = 9.4 Hz, 1H), 7.23 (td, J = 7.5, 0.9 Hz, 1H), 5.36-5.16 (m, 1H), 4.24-4.09 (m, 1H), 3.90-3.73 (m, 3H), 3.68-3.64 (m, 1H), 3.47-3.40 (m, 1H), 3.17 (sept, J = 6.9 Hz, 1H), 1.34-1.26 (m, 15H) |
| | Tert-butyl 3-(4-(2- | | | |
| | fluorophenyl)-3-(2- | | | |
| | isopropylpyrimidine-5- | | | |
| | carboxamido)pyridin-2- | | | |
| | yl)morpholine-4- | | | |
| | carboxylate | | | |
| 555 | 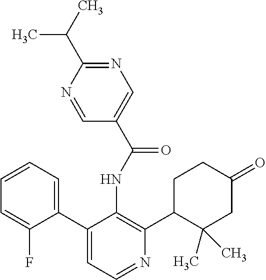 [SMILES: CC(C)c1ncc(C(=O)Nc2c(-c3ccccc3F)ccnc2C2CCC(=O)CC2(C)C)cn1] | 0.023 g 3% | 461.4 (M + 1) | (400 MHz, DMSO-d₆) δ 10.58-10.25 (m, 1H), 8.93 (s, 2H), 8.62 (d, J = 4.9 Hz, 1H), 7.45-7.21 (m, 5H), 3.67-3.39 (m, 1H), 3.24-3.13 (m, 1H), 2.45-2.19 (m, 5H), 2.01-1.97 (m, 1H), 1.28 (d, J = 6.9 Hz, 6H), 0.91 (s, 3H), 0.81-0.77 (m, 3H) |
| | (2-(2,2-dimethyl-4- | | | |
| | oxocyclohexyl)-4-(2- | | | |
| | fluorophenyl)pyridin-3-yl)- | | | |
| | 2-isopropylpyrimidine-5- | | | |
| | carboxamide | | | |
| 556 | 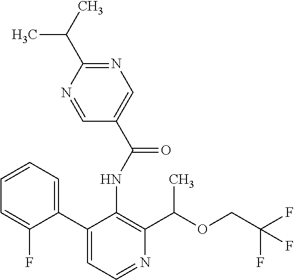 [SMILES: CC(C)c1ncc(C(=O)Nc2c(-c3ccccc3F)ccnc2C(C)OCC(F)(F)F)cn1] | 0.087 g 70% | 463.7 (M + 1) | (400 MHz, DMSO-d₆) δ 10.38 (s, 1H), 8.92 (s, 2H), 8.72 (d, J = 4.9 Hz, 1H), 7.49 (dd, J = 4.9, 0.7 Hz, 1H), 7.47-7.37 (m, 2H), 7.33-7.28 (m, 1H), 7.25 (td, J = 7.5, 1.0 Hz, 1H), 5.08 (q, J = 6.3 Hz, 1H), 3.98-3.77 (m, 2H), 3.18 (sept, J = 6.9 Hz, 1H), 1.49 (d, J = 6.4 Hz, 3H), 1.27 (d, J = 6.9 Hz, 6H) |
| | N-(4-(2-fluorophenyl)-2-(1- | | | |
| | (2,2,2- | | | |
| | trifluoroethoxy)ethyl) | | | |
| | pyridin-3-yl)-2- | | | |
| | isopropylpyrimidine-5- | | | |
| | carboxamide | | | |
| 557 | 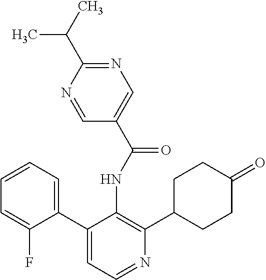 [SMILES: CC(C)c1ncc(C(=O)Nc2c(-c3ccccc3F)ccnc2C2CCC(=O)CC2)cn1] | 0.078 g 67% | 433.2 (M + 1) | (400 MHz, DMSO-d₆) δ 10.39 (s, 1H), 8.96 (s, 2H), 8.60 (d, J = 4.9 Hz, 1H), 7.45-7.35 (m, 3H), 7.32-7.27 (m, 1H), 7.24 (td, J = 7.5, 1.1 Hz, 1H), 3.58-3.51 (m, 1H), 3.19 (sept, J = 7.0 Hz, 1H), 2.59-2.50 (m, 2H), 2.28 (d, J = 14.3 Hz, 2H), 2.08-1.98 (m, 4H), 1.28 (d, J = 6.9 Hz, 6H) |
| | N-(4-(2-fluorophenyl)-2-(4- | | | |
| | oxocyclohexyl) pyridin-3- | | | |
| | yl)-2-isopropylpyrimidine- | | | |
| | 5-carboxamide | | | |
| 558 | 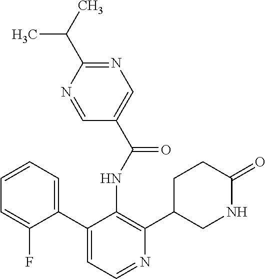 [SMILES: CC(C)c1ncc(C(=O)Nc2c(-c3ccccc3F)ccnc2C2CCC(=O)NC2)cn1] | 0.034 g 15% | 434.2 (M + 1) | (400 MHz, DMSO-d₆) δ 10.39 (s, 1H), 8.93 (s, 2H), 8.62 (d, J = 4.9 Hz, 1H), 7.53 (s, 1H), 7.44-7.35 (m, 3H), 7.32-7.22 (m, 2H), 3.48-3.39 (m, 2H), 3.30-3.29 (m, 1H), 3.18 (sept, J = 6.9 Hz, 1H), 2.26 (t, J = 3.5 Hz, 2H), 2.14- 2.07 (m, 1H), 1.91-1.87 (m, 1H), 1.27 (d, J = 6.9 Hz, 6H) |
| | N-(4-(2-fluorophenyl)-2-(6- | | | |
| | oxopiperidin-3-yl)pyridin-3- | | | |
| | yl)-2-isopropylpyrimidine- | | | |
| | 5-carboxamide | | | |
| 559 | 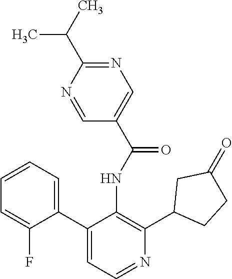 [SMILES: CC(C)c1ncc(C(=O)Nc2c(-c3ccccc3F)ccnc2C2CCC(=O)C2)cn1] | 0.143 g 63% | 419.2 (M + 1) | (400 MHz, DMSO-d₆) δ 10.36 (s, 1H), 8.95 (s, 2H), 8.61 (d, J = 4.9 Hz, 1H), 7.45-7.35 (m, 3H), 7.32-7.22 (m, 2H), 3.94 (dt, J = 12.7, 6.0 Hz, 1H), 3.18 (sept, J = 6.9 Hz, 1H), 2.59-2.47 (m, 2H), 2.34-2.17 (m, 3H), 2.13- 2.04 (m, 1H), 1.27 (d, J = 6.9 Hz, 6H); (376 MHz, DMSO- d₆) δ −114.9 |
| | N-(4-(2-fluorophenyl)-2-(3- | | | |
| | oxocyclopentyl)pyridin-3- | | | |
| | yl)-2-isopropylpyrimidine- | | | |
| | 5-carboxamide | | | |
| 560 | 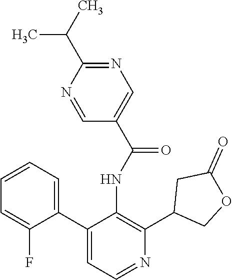 [SMILES: CC(C)c1ncc(C(=O)Nc2c(-c3ccccc3F)ccnc2C2COC(=O)C2)cn1] | 0.016 g 9% | 421.2 (M + 1) | (400 MHz, DMSO-d₆) δ 10.39 (s, 1H), 8.95 (s, 2H), 8.65 (d, J = 4.9 Hz, 1H), 7.47-7.41 (m, 2H), 7.39-7.24 (m, 3H), 4.59 (t, J = 8.1 Hz, 1H), 4.34-4.30 (m, 1H), 4.29- 4.22 (m, 1H), 3.24-3.14 (m, 1H), 2.88 (dd, J = 17.1, 8.7 Hz, 1H), 2.77 (dd, J = 17.2, 5.6 Hz, 1H), 1.27 (d, J = 6.9 Hz, 6H) |
| | N-(4-(2-fluorophenyl)-2-(5- | | | |
| | oxotetrahydrofuran-3- | | | |
| | yl)pyridin-3-yl)-2- | | | |
| | isopropylpyrimidine-5- | | | |
| | carboxamide | | | |
| 561 | 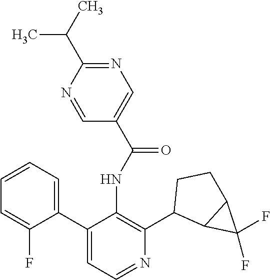 [SMILES: CC(C)c1ncc(C(=O)Nc2c(-c3ccccc3F)ccnc2C2CCC3C2C3(F)F)cn1] | 0.020 g 11% | 453.2 (M + 1) | (400 MHz, DMSO-d₆) δ 10.40 (s, 1H), 8.91 (s, 2H), 8.67 (d, J = 4.9 Hz, 1H), 7.45-7.39 (m, 3H), 7.31-7.22 (m, 2H), 6.33-6.29 (m, 1H), 5.86-5.84 (m, 1H), 3.85 (s, 1H), 3.17 (sept, J = 6.9 Hz, 1H), 2.47-2.28 (m, 2H), 2.28- 2.14 (m, 1H), 1.94-1.89 (m, 1H), 1.27 (d, J = 6.9 Hz, 6H) |
| | N-(2-(6,6- | | | |
| | difluorobicyclo[3.1.0]hexan- | | | |
| | 2-yl)-4-(2- | | | |
| | fluorophenyl)pyridin-3-yl)- | | | |
| | 2-isopropylpyrimidine-5- | | | |
| | carboxamide | | | |
| 562 | 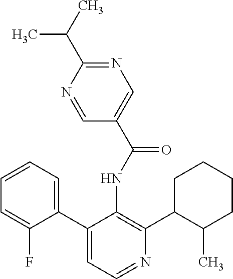 [SMILES: CC(C)c1ncc(C(=O)Nc2c(-c3ccccc3F)ccnc2C2CCCCC2C)cn1] | 0.036 g 15% | 433.3 (M + 1) | (400 MHz, DMSO-d₆) δ 10.29 (s, 1H), 8.90 (s, 2H), 8.60 (d, J = 4.9 Hz, 1H), 7.42-7.35 (m, 2H), 7.31-7.26 (m, 2H), 7.24-7.20 (m, 1H), 3.18 (sept, J = 6.9 Hz, 1H), 2.67-2.61 (m, 1H), 2.01-1.90 (m, 1H), 1.74 (dt, J = 25.8, 13.6 Hz, 4H), 1.56-1.42 (m, 1H), 1.38-1.17 (m, 8H), 1.02 (q, J = 12.1 Hz, 1H), 0.60- 0.59 (m, 3H) |
| | N-(4-(2-fluorophenyl)-2-(2- | | | |
| | methylcyclohexyl)pyridin- | | | |
| | 3-yl)-2- | | | |
| | isopropylpyrimidine-5- | | | |
| | carboxamide | | | |
| 563 | 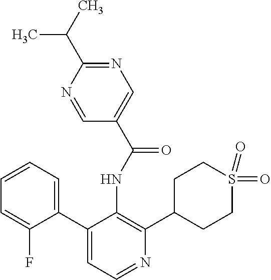 [SMILES: CC(C)c1ncc(C(=O)Nc2c(-c3ccccc3F)ccnc2C2CCS(=O)(=O)CC2)cn1] | 0.051 g 40% | 469.2 (M + 1) | (400 MHz, DMSO-d₆) δ 10.33 (s, 1H), 8.93 (s, 2H), 8.63 (d, J = 4.9 Hz, 1H), 7.46-7.40 (m, 1H), 7.39-7.28 (m, 3H), 7.24 (td, J = 7.5, 1.1 Hz, 1H), 3.46-3.34 (m, 3H), 3.20 (sept, J = 6.9 Hz, 1H), 3.11 (t, J = 12.4 Hz, 2H), 2.33 (q, J = 12.6 Hz, 2H), 2.07 (d, J = 14.3 Hz, 2H), 1.28 (d, J = 6.9 Hz, 6H) |
| | N-(2-(1,1- | | | |
| | dioxidotetrahydro-2H- | | | |
| | thiopyran-4-yl)-4-(2- | | | |
| | fluorophenyl)pyridin-3-yl)- | | | |
| | 2-isopropylpyrimidine-5- | | | |
| | carboxamide | | | |
| 564 | 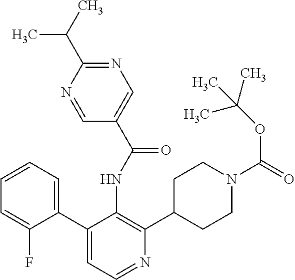 [SMILES: CC(C)c1ncc(C(=O)Nc2c(-c3ccccc3F)ccnc2C2CCN(C(=O)OC(C)(C)C)CC2)cn1] | 0.251 g 90% | 520.2 (M + 1) | (400 MHz, DMSO-d₆) δ 10.33 (s, 1H), 8.92 (s, 2H), 8.60 (d, J = 4.9 Hz, 1H), 7.44-7.21 (m, 5H), 4.04 (d, J = 8.6 Hz, 2H), 3.23-3.15 (m, 2H), 2.84-2.73 (m, 2H), 1.70 (dd, J = 0.7, 0.3 Hz, 4H), 1.41 (s, 9H), 1.27 (d, J = 6.9 Hz, 6H) |
| | Tert-butyl 4-(4-(2- | | | |
| | fluorophenyl)-3-(2- | | | |
| | isopropylpyrimidine-5- | | | |
| | carboxamido)pyridin-2- | | | |
| | yl)piperidine-1-carboxylate | | | |
| 565 | 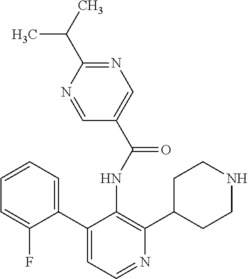 [SMILES: CC(C)c1ncc(C(=O)Nc2c(-c3ccccc3F)ccnc2C2CCNCC2)cn1] | 0.138 g 61% | 420.2 (M + 1) | (400 MHz, DMSO-d₆) δ 10.55 (s, 1H), 8.94 (s, 2H), 8.62 (d, J = 4.9 Hz, 1H), 8.42 (s, 1H), 7.43-7.35 (m, 3H), 7.31-7.26 (m, 1H), 7.22 (td, J = 7.5, 1.1 Hz, 1H), 3.32-3.13 (m, 4H), 2.86-2.83 (m, 2H), 1.97 (q, J = 11.5 Hz, 2H), 1.81 (d, J = 13.2 Hz, 2H), 1.27 (d, J = 6.9 Hz, 6H) |
| | Tert-butyl 4-(4-(2- | | | |
| | fluorophenyl)-3-(2- | | | |
| | isopropylpyrimidine-5- | | | |
| | carboxamido)pyridin-2- | | | |
| | yl)piperidine-1-carboxylate | | | |
| 566 | 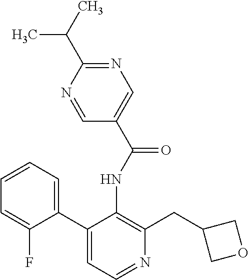 [SMILES: CC(C)c1ncc(C(=O)Nc2c(-c3ccccc3F)ccnc2CC2COC2)cn1] | 0.034 g 21% | 407.2 (M + 1) | (400 MHz, DMSO-d₆) δ 10.34 (s, 1H), 8.96 (s, 2H), 8.53 (d, J = 5.0 Hz, 1H), 7.44-7.39 (m, 1H), 7.38-7.34 (m, 2H), 7.29 (t, J = 9.2 Hz, 1H), 7.25-7.22 (m, 1H), 4.71 (dd, J = 7.8, 5.9 Hz, 2H), 4.36 (t, J = 6.2 Hz, 2H), 3.48 (s, 1H), 3.26-3.12 (m, 3H), 1.27 (d, J = 6.9 Hz, 6H) |
| | N-(4-(2-fluorophenyl)-2- | | | |
| | (oxetan-3-ylmethyl)pyridin- | | | |
| | 3-yl)-2- | | | |
| | isopropylpyrimidine-5- | | | |
| | carboxamide | | | |
| 567 | 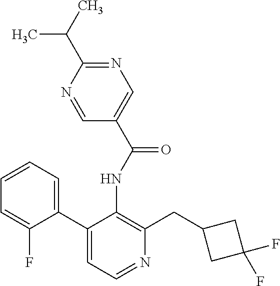 [SMILES: CC(C)c1ncc(C(=O)Nc2c(-c3ccccc3F)ccnc2CC2CC(F)(F)C2)cn1] | 0.067 g 38% | 441.2 (M + 1) | (400 MHz, DMSO-d₆) δ 10.34 (s, 1H), 8.94 (s, 2H), 8.58 (d, J = 5.0 Hz, 1H), 7.44-7.34 (m, 3H), 7.31-7.29 (m, 1H), 7.24 (td, J = 7.5, 0.9 Hz, 1H), 3.18 (sept, J = 6.9 Hz, 1H), 3.04 (d, J = 6.0 Hz, 2H), 2.73-2.61 (m, 3H), 2.41- 2.27 (m, 2H), 1.27 (d, J = 6.9 Hz, 6H) |
| | N-(2-((3,3- | | | |
| | difluorocyclobutyl)methyl)- | | | |
| | 4-(2-fluorophenyl)pyridin- | | | |
| | 3-yl)-2- | | | |
| | isopropylpyrimidine-5- | | | |
| | carboxamide | | | |
| 568 | 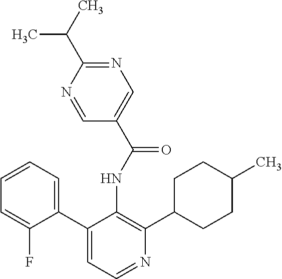 [SMILES: CC1CCC(c2nccc(-c3ccccc3F)c2NC(=O)c2cnc(C(C)C)nc2)CC1] | 0.124 g 53% | 434.2 (M + 1) | (400 MHz, DMSO-d₆, dr = 2:1) δ 10.30 (s, 3H), 8.92 (s, 6H), 8.61 (d, J = 4.9 Hz, 1H), 8.58 (d, J = 4.9 Hz, 2H), 7.42-7.34 (m, 6H), 7.30-7.27 (m, 6H), 7.25-7.20 (m, 3H), 3.18 (sept, J = 6.9 Hz, 3H), 3.04-2.90 (m, 3H), 1.90-1.36 (m, 24H), 1.27 (d, J = 6.9 Hz, 18H), 1.08-0.91 (m, 6H), 0.87 (d, J = 6.5 Hz, 6H); (376 MHz, DMSO-d₆) δ −114.9, −115.0 |
| | N-(4-(2-fluorophenyl)-2-(4- | | | |
| | methylcyclohexyl)pyridin- | | | |
| | 3-yl)-2- | | | |
| | isopropylpyrimidine-5- | | | |
| | carboxamide | | | |
| | dr = 2:1 | | | |

### Example 569-571

In a similar manner as described in examples disclosed herein, utilizing the appropriately substituted starting materials and intermediates, the following compounds were prepared:

| Example | Structure | Amount | MS (ES+) | |
|---|---|---|---|---|
| No. | Name | Yield | m/z | ¹H NMR; ¹⁹F NMR |
| 569 | 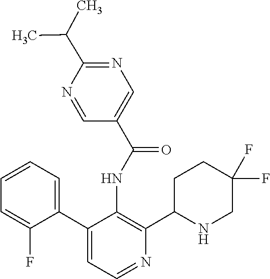 [SMILES: CC(C)c1ncc(C(=O)Nc2c(-c3ccccc3F)ccnc2C2CCC(F)(F)CN2)cn1] | 0.328 g 98% | 456.0 (M + 1) | (400 MHz, DMSO-d₆) δ 10.81 (s, 1H), 9.05 (s, 2H), 8.77 (d, J = 5.0 Hz, 1H), 7.64 (dd, J = 4.9, 0.9 Hz, 1H), 7.46-7.41 (m, 2H), 7.35-7.30 (m, 1H), 7.28-7.22 (m, 1H), 4.84 (d, J = 10.7 Hz, 1H), 3.72-3.58 (m, 2H), 3.25-3.11 (m, J = 6.5 Hz, 1H), 2.40- 2.27 (m, 3H), 2.01 (s, 1H), 1.32-1.22 (m, 6H); (376 MHz, DMSO-d₆) δ-100.0, -115.1 |
| | N-(2-(5,5-difluoropiperidin-2-yl)-4-(2- | | | |
| | fluorophenyl)pyridin-3-yl)-2- | | | |
| | isopropylpyrimidine-5-carboxamide | | | |
| 570 | 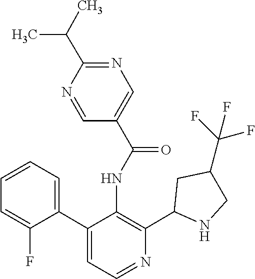 [SMILES: CC(C)c1ncc(C(=O)Nc2c(-c3ccccc3F)ccnc2C2CC(C(F)(F)F)CN2)cn1] | 0.212 g 87% | 474.2 (M + 1) | (400 MHz, DMSO-d₆) δ 10.92 (s, 1H), 10.58 (s, 1H), 9.45 (s, 1H), 9.03 (s, 2H), 8.75 (d, J = 5.0 Hz, 1H), 7.67-7.65 (m, 1H), 7.49-7.43 (m, 2H), 7.35-7.26 (m, 2H), 5.20 (s, 1H), 3.85 (t, J = 10.2 Hz, 1H), 3.61 (dq, J = 16.0, 8.0 Hz, 1H), 3.20 (sept, J = 6.9 Hz, 1H), 2.63-2.55 (m, 1H), 2.40-2.33 (m, 1H), 1.29 (d, J = 6.9 Hz, 6H); (376 MHz, DMSO-d₆) δ-69.5, -115.0 |
| | N-(4-(2-fluorophenyl)-2-(4-(trifluoromethyl) | | | |
| | pyrrolidin-2-yl)pyridin-3-yl)-2- | | | |
| | isopropylpyrimidine-5-carboxamide | | | |
| 571 | 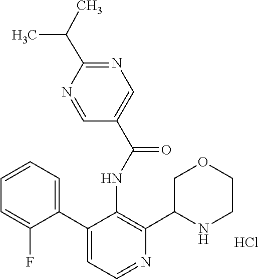 [SMILES: CC(C)c1ncc(C(=O)Nc2c(-c3ccccc3F)ccnc2C2COCCN2)cn1 ; Cl] | 0.222 g 92% | 422.2 (M + 1) | (400 MHz, DMSO-d₆) δ 10.86 (s, 1H), 9.92 (s, 1H), 9.51-9.40 (m, 1H), 9.05 (s, 2H), 8.78 (d, J = 5.0 Hz, 1H), 7.67 (dd, J = 5.0, 0.9 Hz, 1H), 7.47-7.42 (m, 2H), 7.34- 7.24 (m, 2H), 4.93-4.87 (m, 1H), 4.34-4.27 (m, 1H), 3.99 (dd, J = 12.2, 1.9 Hz, 1H), 3.80 (td, J = 12.1, 2.1 Hz, 1H), 3.62 (t, J = 11.3 Hz, 1H), 3.32-3.24 (m, 2H), 3.19 (sept, J = 6.9 Hz, 1H), 1.28 (d, J = 6.9 Hz, 6H) |
| | N-(4-(2-fluorophenyl)-2- | | | |
| | (morpholin-3-yl)pyridin-3- | | | |
| | yl)-2-isopropylpyrimidine-5- | | | |
| | carboxamide hydrochloric acid salt | | | |

### Example 572-575

In a similar manner as described in Example 43, utilizing the appropriately substituted starting materials and intermediates, the following compounds were prepared:

| Example | Structure | Amount | MS (ES+) | |
|---|---|---|---|---|
| No. | Name | Yield | m/z | ¹H NMR; ¹⁹F NMR |
| 572 | 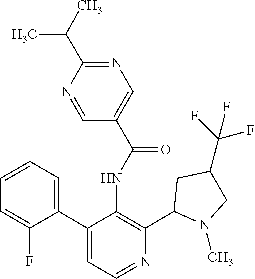 [SMILES: CC(C)c1ncc(C(=O)Nc2c(-c3ccccc3F)ccnc2C2CC(C(F)(F)F)CN2C)cn1] | 0.071 g 46% | 488.2 (M + 1) | (400 MHz, DMSO-d₆) δ 10.46 (s, 1H), 8.91 (s, 2H), 8.66 (d, J = 4.9 Hz, 1H), 7.44-7.37 (m, 3H), 7.31-7.19 (m, 2H), 3.89 (t, J =7.7 Hz, 1H), 3.35-3.13 (m, 3H), 2.42- 2.31 (m, 2H), 2.22-2.15 (m, 1H), 2.08 (s, 3H), 1.28 (d, J = 6.9 Hz, 6H); (376 MHz, DMSO-d₆) δ-69.4, -115.0 |
| | N-(4-(2-fluorophenyl)-2-(1-methyl-4- | | | |
| | (trifluoromethyl)pyrrolidin-2-yl)pyridin-3- | | | |
| | 2-yl)-isopropylpyrimidine-5-carboxamide | | | |
| 573 | 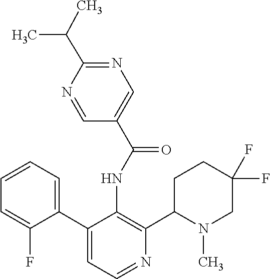 [SMILES: CC(C)c1ncc(C(=O)Nc2c(-c3ccccc3F)ccnc2C2CCC(F)(F)CN2C)cn1] | 0.028 g 27% | 470.2 (M + 1) | (400 MHz, DMSO-d₆) δ 10.59 (s, 1H), 8.93 (s, 2H), 8.61 (d, J = 4.9 Hz, 1H), 7.42-7.37 (m, 3H), 7.30-7.25 (m, 1H), 7.21 (td, J = 7.5, 0.9 Hz, 1H), 3.64-3.62 (m, 1H), 3.28-3.13 (m, 2H), 2.59-2.50 (m, 1H), 2.17-2.09 (m, 1H), 2.00-1.85 (m, 3H), 2.03 (s, 3H), 1.27 (d, J = 6.9 Hz, 6H) |
| | N-(2-(5,5-difluoro-1-methylpiperidin-2-yl)- | | | |
| | 4-(2-fluorophenyl)pyridin-3-yl)-2- | | | |
| | isopropylpyrimidine-5-carboxamide | | | |
| 574 | 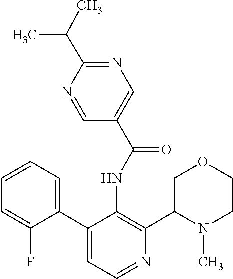 [SMILES: CC(C)c1ncc(C(=O)Nc2c(-c3ccccc3F)ccnc2C2COCCN2C)cn1] | 0.051 g 27% | 436.2 (M + 1) | (400 MHz, DMSO-d₆) δ 10.62 (s, 1H), 8.95 (s, 2H), 8.63 (d, J = 4.9 Hz, 1H), 7.42-7.36 (m, 3H), 7.31-7.21 (m, 2H), 3.78-3.74 (m, 2H), 3.64-3.50 (m, 3H), 3.19 (sept, J = 6.9 Hz, 1H), 2.79 (d, J = 11.8 Hz, 1H), 2.23 (td, J = 11.6, 3.0 Hz, 1H), 1.96 (s, 3H), 1.28 (d, J = 6.9 Hz, 6H) |
| | N-(4-(2-fluorophenyl)-2-(4-methylmorpholin- | | | |
| | 3-yl)pyridin-3-yl)-2- | | | |
| | isopropylpyrimidine-5-carboxamide | | | |
| 575 | 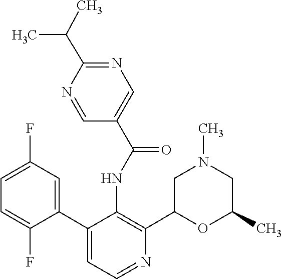 [SMILES: CC(C)c1ncc(C(=O)Nc2c(-c3cc(F)ccc3F)ccnc2C2CN(C)C[C@@H](C)O2)cn1] | 0.045 g 60% | 468.2 (M + 1) | (400 MHz, CD₃OD, dr = 4.5:1) δ 8.99 (s, 2H), 8.98 (s, 9H), 8.68 (d, J = 4.6 Hz, 1H), 8.67 (d, J = 4.6 Hz, 4.5H), 7.52 (d, J = 5.0 Hz, 1H), 7.49 (d, J = 5.0 Hz, 4.5H), 7.26-7.14 (m, 16.5H), 5.31 (t, J = 5.3 Hz, 4.5H), 5.01 (dd, J = 10.4, 2.2 Hz, 1H), 4.06 (qt, J = 6.7, 3.5 Hz, 4.5H), 3.79 (dqd, J = 10.1, 6.5, 2.5 Hz, 1H), 3.24 (sept, J = 6.9 Hz, 5.5H), 2.98 (d, J = 11.4 Hz, 1H), 2.82-2.75 (m, 10H), 2.47 (qd, J = 12.5, 3.6 Hz, 9H), 2.38 (t, J = 11.1 Hz, 1H), 2.33 (s, 3H), 2.29 (s, 13.5H), 1.92 (t, J = 11.0 Hz, 1H), 1.35 (d, J = 6.9 Hz, 33H), 1.26 (d, J = 6.6 Hz, 13.5H), 1.19 (d, J = 6.3 Hz, 3H) |
| | N-(4-(2,5-difluorophenyl)-2-((6R)-4,6- | | | |
| | dimethylmorpholin-2-yl)pyridin-3-yl)-2- | | | |
| | isopropylpyrimidine-5-carboxamide | | | |
| | dr = 4.5:1 | | | |

### Example 576

### Synthesis of N-(2-(5,5-difluoro-1-(2,2,2-trifluoroethyl)piperidin-2-yl)-4-(2-fluorophenyl)pyridin-3-yl)-2-isopropylpyrimidine-5-carboxamide

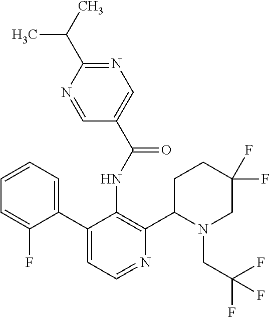 [SMILES: CC(C)c1ncc(C(=O)Nc2c(-c3ccccc3F)ccnc2C2CCC(F)(F)CN2CC(F)(F)F)cn1]

To a mixture of N-(2-(5,5-difluoropiperidin-2-yl)-4-(2-fluorophenyl)pyridin-3-yl)-2-isopropylpyrimidine-5-carboxamide (0.063 g, 0.14 mmol) in anhydrous tetrahydrofuran (1.4 mL) was added N,N-diisopropylethylamine (0.24 mL, 1.38 mmol) and 2,2,2-trifluoroethyl trifluoromethanesulfonate (0.10 mL, 0.69 mmol). The reaction mixture was stirred at 65° C. for 5 h. After cooling to ambient temperature, the reaction mixture was diluted with ethyl acetate (75 mL), washed with saturated ammonium chloride (2×25 mL), dried over anhydrous magnesium sulfate, filtered, and concentrated in vacuo. Purification of the residue by preparative reverse phase HPLC, eluting with a gradient of 15-90% acetonitrile in water containing 0.5% formic acid, afforded the title compound as a colorless solid (0.026 g, 35% yield): ¹H NMR (400 MHz, DMSO-d₆) δ 10.40 (s, 1H), 8.89 (s, 2H), 8.68 (d, J=4.9 Hz, 1H), 7.47-7.36 (m, 3H), 7.33-7.22 (m, 2H), 4.31-4.30 (m, 1H), 3.81 (q, J=12.7 Hz, 1H), 3.24-3.08 (m, 3H), 3.08-2.98 (m, 1H), 2.29-2.15 (m, 1H), 2.07-1.92 (m, 3H), 1.27 (d, J=6.9 Hz, 6H); ¹⁹F NMR (400 MHz, DMSO-d₆) δ −69.2, −98.6, −114.9; MS (ES+) m/z 538.2 (M+1).

### Example 577

In a similar manner as described in Example 576, utilizing the appropriately substituted starting materials and intermediates, the following compounds were prepared:

| Example | | Amount | MS (ES+) | |
|---|---|---|---|---|
| No. | Structure | Yield | m/z | ¹H NMR; ¹⁹F NMR |
| 577 | 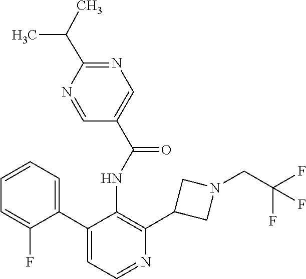 [SMILES: CC(C)c1ncc(C(=O)Nc2c(-c3ccccc3F)ccnc2C2CN(CC(F)(F)F)C2)cn1] | 0.023 g 42% | 474.2 (M + 1) | (400 MHz, DMSO-d₆) δ 10.27 (s, 1H), 8.94 (s, 2H), 8.65 (d, J = 5.0 Hz, 1H), 7.46-7.39 (m, 2H), 7.37-7.22 (m, 3H), 4.10 (quintet, J = 8.0 Hz, 1H), 3.70 (t, J = 7.3 Hz, 2H), 3.56 (s, 2H), 3.24- 3.15 (m, 3H), 1.28 (d, J = 6.9 Hz, 6H); (376 MHz, DMSO-d₆) δ-69.7, -115.0 |
| | N-(4-(2-fluorophenyl)-2-(1-(2,2,2- | | | |
| | trifluoroethyl)azetidin-3-yl)pyridin-3-yl)-2- | | | |
| | isopropylpyrimidine-5-carboxamide | | | |

### Example 578

### Synthesis of N-(2-(1-acetyl-5,5-difluoropiperidin-2-yl)-4-(2-fluorophenyl)pyridin-3-yl)-2-isopropylpyrimidine-5-carboxamide

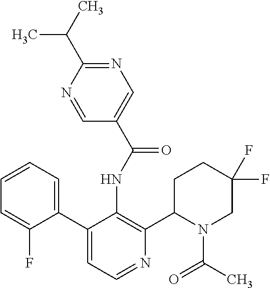 [SMILES: CC(=O)N1CC(F)(F)CCC1c1nccc(-c2ccccc2F)c1NC(=O)c1cnc(C(C)C)nc1]

To a mixture of N-(2-(5,5-difluoropiperidin-2-yl)-4-(2-fluorophenyl)pyridin-3-yl)-2-isopropylpyrimidine-5-carboxamide (0.063 g, 0.14 mmol) in anhydrous tetrahydrofuran (1.4 mL) was added N,N-diisopropylethylamine (0.24 mL, 1.4 mmol) and acetyl chloride (0.01 mL, 0.2 mmol). The reaction mixture was stirred at ambient temperature for 2.5 h. The reaction mixture was diluted with ethyl acetate (75 mL) and the organic phase was washed with saturated ammonium chloride (2×25 mL), dried over anhydrous magnesium sulfate, filtered, and concentrated in vacuo. Purification of the residue by preparative reverse phase HPLC, eluting with a gradient of 15 to 90% acetonitrile in water containing 0.5% formic acid, afforded the title compound as a colorless solid (0.029 g, 42% yield): ¹H NMR (400 MHz, DMSO-d₆, rotamers are present) δ 10.42 (s, 0.4H), 10.37 (s, 0.6H), 8.92 (s, 0.8H), 8.90 (s, 1.2H), 8.65 (d, J=4.9 Hz, 1H), 7.51-7.49 (m, 1H), 7.47-7.35 (m, 2H), 7.33-7.20 (m, 2H), 6.05 (s, 0.6H), 5.48 (s, 0.4H), 4.71 (t, J=14.4 Hz, 0.4H), 4.08 (t, J=12.9 Hz, 0.6H), 3.89 (ddd, J=34.0, 13.9, 3.8 Hz, 0.4H), 3.71-3.56 (m, 0.6H), 3.18 (sept, J=6.8 Hz, 1H), 2.84-2.65 (m, 0.6H), 2.30-2.05 (m, 3H), 1.97-1.91 (m, 3.4H), 1.28 (dd, J=6.9, 1.7 Hz, 6H); MS (ES+) m/z 498.2 (M+1).

### Example 579

### Synthesis of N-(4-(2-fluorophenyl)-2-(1-(oxetan-3-yl)azetidin-3-yl)pyridin-3-yl)-2-isopropylpyrimidine-5-carboxamide

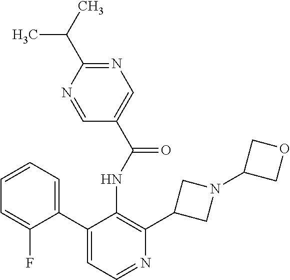 [SMILES: CC(C)c1ncc(C(=O)Nc2c(-c3ccccc3F)ccnc2C2CN(C3COC3)C2)cn1]

### Step 1. Preparation of tert-butyl 3-(4-(2-fluorophenyl)-3-(2-isopropylpyrimidine-5-carboxamido)pyridin-2-yl)azetidine-1-carboxylate

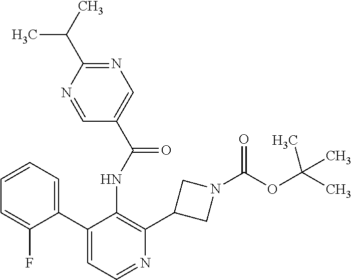 [SMILES: CC(C)c1ncc(C(=O)Nc2c(-c3ccccc3F)ccnc2C2CN(C(=O)OC(C)(C)C)C2)cn1]

To a vial containing N-(2-chloro-4-(2-difluorophenyl)pyridin-3-yl)-2-isopropylpyrimidine-5-carboxamide (0.300 g, 0.809 mmol), cesium carbonate (0.554 g, 1.70 mmol), nickel(II) chloride dimethoxyethane adduct (0.018 g, 0.081 mmol), 4-tert-butyl-2-(4-tert-butyl-2-pyridyl)pyridine (0.033 g, 0.12 mmol), (4,4″-di-tert-butyl-2,2″-bipyridine)bis[3,5-difluoro-2-[5-trifluoromethyl-2-pyridinyl-kN)phenyl-kC]iridium(II) hexafluorophosphate (0.009 g, 0.008 mmol), 1-(tert-butoxycarbonyl)azetidine-3-carboxylic acid (0.326 g, 1.62 mmol) was added anhydrous N,N-dimethylformamide (20.0 mL) and the headspace was flushed with nitrogen for 10 seconds. The vial was sealed and placed in front of 4 Kessil PR160L lights (440 nm) for 24 h. To the reaction mixture was added cesium carbonate (0.277 g, 0.850 mmol), 1-(tert-butoxycarbonyl)azetidine-3-carboxylic acid (0.155 g, 0.771 mmol) and the headspace was flushed with nitrogen for 10 seconds. The vial was sealed and placed in front of 4 Kessil PR160L lights (440 nm) for 8 h. The reaction mixture was diluted with ethyl acetate (150 mL) and the organic phase was washed with saturated sodium bicarbonate (50 mL), saturated ammonium chloride (2×50 mL), water (50 mL), and brine (50 mL), dried over anhydrous magnesium sulfate, filtered, and concentrated in vacuo. The residue was purified by column chromatography, using a gradient of 15 to 100% of ethyl acetate in heptane as eluent, to afford the title compound as a colorless oil (0.138 g, 35% yield): MS (ES+) m/z 492.2 (M+1).

### Step 2. Preparation of N-(2-(azetidin-3-yl)-4-(2-fluorophenyl)pyridin-3-yl)-2-isopropylpyrimidine-5-carboxamide hydrochloric acid salt

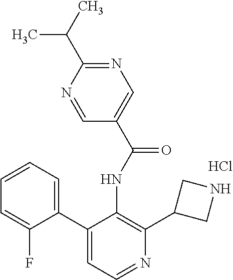 [SMILES: CC(C)c1ncc(C(=O)Nc2c(-c3ccccc3F)ccnc2C2CNC2)cn1 ; Cl]

To a mixture of tert-butyl 3-(4-(2-fluorophenyl)-3-(2-isopropylpyrimidine-5-carboxamido)pyridin-2-yl)azetidine-1-carboxylate (0.138 g, 0.347 mmol) in anhydrous 1,4-dioxane (4.0 mL) was added 4.0 M hydrochloric acid in anhydrous dioxane (2.0 mL, 2.0 mmol). The reaction mixture was stirred at ambient temperature for 3 h. The reaction mixture was concentrated in vacuo, to afford the title compound as a crude colorless oil (0.161 g, quantitative yield): MS (ES+) m/z 392.0 (M+1).

### Step 3. Preparation of N-(4-(2-fluorophenyl)-2-(1-(oxetan-3-yl)azetidin-3-yl)pyridin-3-yl)-2-isopropylpyrimidine-5-carboxamide formic acid salt

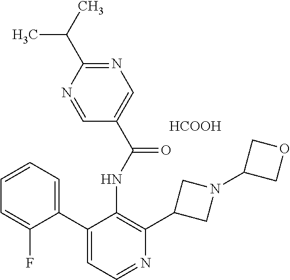 [SMILES: CC(C)c1ncc(C(=O)Nc2c(-c3ccccc3F)ccnc2C2CN(C3COC3)C2)cn1 ; "HCOOH"]

To a mixture of N-(2-(azetidin-3-yl)-4-(2-fluorophenyl)pyridin-3-yl)-2-isopropylpyrimidine-5-carboxamide hydrochloric acid salt (0.050 g, 0.18 mmol) in anhydrous dichloromethane (2.3 mL) was added 3-oxetanone (0.042 g, 0.58 mmol) and acetic acid (0.035 g, 0.58 mmol). The reaction mixture was left to stir at ambient temperature for 30 minutes. To the reaction mixture was added sodium triacetoxyborohydride (0.248 g, 1.17 mmol). The reaction mixture was stirred at ambient temperature for 24 h. The reaction mixture was diluted with ethyl acetate (75 mL) and the organic phase was washed with saturated sodium bicarbonate (2×50 mL), dried over anhydrous magnesium sulfate, filtered, and concentrated in vacuo. Purification of the residue by preparative reverse phase HPLC, using 10 to 60% acetonitrile in water containing 0.5% of formic acid as eluent, afforded the title compound as a colorless solid (0.017 g, 33% yield): ¹H NMR (400 MHz, DMSO-d₆) δ 10.28 (s, 1H), 8.94 (s, 2H), 8.64 (d, J=5.0 Hz, 1H), 8.15 (s, 0.18H), 7.45-7.22 (m, 5H), 4.54 (t, J=6.6 Hz, 2H), 4.37 (t, J=5.8 Hz, 2H), 4.06 (quintet, J=7.9 Hz, 1H), 3.70 (tt, J=6.6, 5.3 Hz, 1H), 3.58 (t, J=7.5 Hz, 2H), 3.45-3.40 (m, 2H), 3.19 (sept, J=6.9 Hz, 1H), 1.28 (d, J=6.9 Hz, 6H); MS (ES+) m/z 448.2 (M+1).

### Example 580

### Synthesis of N-(4-(2,5-difluorophenyl)-2-(4-hydroxycyclohexyl)pyridin-3-yl)-2-isopropylpyrimidine-5-carboxamide

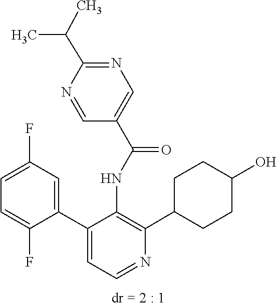 [SMILES: CC(C)c1ncc(C(=O)Nc2c(-c3cc(F)ccc3F)ccnc2C2CCC(O)CC2)cn1]

### Step 1. Preparation of 2-chloro-4-(2,5-difluorophenyl)pyridin-3-amine hydrochloric acid salt

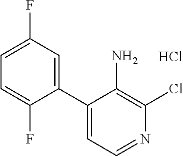 [SMILES: Nc1c(-c2cc(F)ccc2F)ccnc1Cl ; Cl]

To a mixture of tert-butyl (2-chloro-4-(2,5-difluorophenyl)pyridin-3-yl)carbamate (6.00 g, 17.6 mmol) in anhydrous dichloromethane (35.0 mL) was added 4.0 M hydrochloric acid in anhydrous dioxane (44.0 mL, 176 mmol). The reaction mixture was stirred at 40° C. for 1.5 h. After cooling to ambient temperature, the reaction mixture was concentrated in vacuo, to afford the title compound as a crude red oil (4.88 g, quantitative yield): MS (ES+) m/z 241.2 (M+1), 243.2 (M+1).

### Step 2. Preparation of N-(2-chloro-4-(2,5-difluorophenyl)pyridin-3-yl)-2-isopropylpyrimidine-5-carboxamide

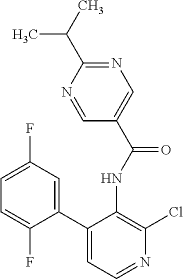 [SMILES: CC(C)c1ncc(C(=O)Nc2c(-c3cc(F)ccc3F)ccnc2Cl)cn1]

To a mixture of 2-chloro-4-(2,5-difluorophenyl)pyridin-3-amine hydrochloric acid salt (4.88 g, 17.6 mmol) in anhydrous tetrahydrofuran (176 mL) and pyridine (28.5 mL, 352 mmol) was added 2-chloro-1-methylpyridinium iodide (18.0 g, 70.4 mmol) and 2-isopropylpyrimidine-5-carboxylic acid (3.51 g, 21.1 mmol). The reaction mixture was stirred at 65° C. for 3 days. After cooling to ambient temperature, the reaction mixture was diluted with methanol (20 mL) and 5 M sodium hydroxide (10 mL). The reaction mixture was stirred at ambient temperature for 10 minutes. The reaction mixture was diluted with ethyl acetate (300 mL), and the organic phase was washed with water (100 mL), 1 M hydrochloric acid (2×100 mL), saturated potassium carbonate (100 mL), dried over anhydrous magnesium sulfate, filtered, and concentrated in vacuo. The residue was purified by column chromatography, eluting with a gradient of 10 to 50% of ethyl acetate in heptane, to afford the title compound as a colorless solid (3.14 g, 46% yield): ¹H NMR (400 MHz, CDCl₃) δ 8.99 (s, 2H), 8.47 (d, J=5.0 Hz, 1H), 7.71 (s, 1H), 7.33 (d, J=5.0 Hz, 1H), 7.15-7.04 (m, 3H), 3.29 (sept, J=6.9 Hz, 1H), 1.36 (d, J=6.9 Hz, 6H); ¹⁹F NMR (400 MHz, CDCl₃) δ −116.8 (d, J=17 Hz), −120.9 (d, J=17 Hz); MS (ES+) m/z 241.2 (M+1), 243.2 (M+1).

### Step 3. Preparation of N-(4-(2-fluorophenyl)-2-(4-hydroxycyclohexyl)pyridin-3-yl)-2-isopropylpyrimidine-5-carboxamide

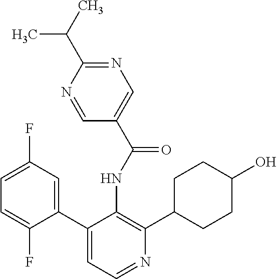 [SMILES: CC(C)c1ncc(C(=O)Nc2c(-c3cc(F)ccc3F)ccnc2C2CCC(O)CC2)cn1]

To a vial containing N-(2-chloro-4-(2,5-difluorophenyl)pyridin-3-yl)-2-isopropylpyrimidine-5-carboxamide (0.250 g, 0.643 mmol), cesium carbonate (0.440 g, 1.35 mmol), nickel(II) chloride dimethoxyethane adduct (0.014 g, 0.064 mmol), 4-tert-butyl-2-(4-tert-butyl-2-pyridyl)pyridine (0.026 g, 0.097 mmol), (4,4″-di-tert-butyl-2,2″-bipyridine)bis[3,5-difluoro-2-[5-trifluoromethyl-2-pyridinyl-kN)phenyl-kC]iridium(II) hexafluorophosphate (0.007 g, 0.006 mmol), and 4-hydroxycyclohexanecarboxylic acid (0.185 g, 1.29 mmol) was added anhydrous N,N-dimethylformamide (16.0 mL) and the headspace was flushed with nitrogen for 10 seconds. The vial was sealed and placed in front of 4 Kessil PR160L lights (440 nm) for 16 h. The reaction mixture was diluted with ethyl acetate (150 mL) and the organic phase was washed with saturated sodium bicarbonate (50 mL), water (3×50 mL), brine (50 mL), dried over anhydrous magnesium sulfate, filtered, and concentrated in vacuo. The residue was purified by column chromatography, using a gradient of 20 to 100% of ethyl acetate in heptane as eluent, to afford the title compound as a colorless solid (0.132 g, 46% yield): ¹H NMR (400 MHz, CD₃CN, dr=2:1) δ 8.89 (s, 4H), 8.89 (s, 2H), 8.66 (s, 3H), 8.63 (d, J=4.9 Hz, 1H), 8.59 (d, J=4.9 Hz, 2H), 7.26 (d, J=4.9 Hz, 3H), 7.20-7.08 (m, 9H), 3.99-3.92 (m, 1H), 3.60-3.51 (m, 2H), 3.22 (sept, J=6.9 Hz, 3H), 3.08-2.94 (m, 3H), 2.88 (s, 2H), 2.76 (s, 1H), 2.74 (d, J=4.5 Hz, 2H), 2.57 (d, J=3.0 Hz, 1H), 2.09 (qd, J=12.9, 3.8 Hz, 2H), 2.00-1.90 (m, 4H), 1.83-1.71 (m, 9H), 1.59-1.47 (m, 3H), 1.31 (d, J=6.9 Hz, 18H), 1.28-1.20 (mn, 3H); MS (ES+) m/z 453.4 (M+1).

### Example 581-612

In a similar manner as described in Example 580, utilizing the appropriately substituted starting materials and intermediates, the following compounds were prepared:

| Example | Structure | Amount | MS (ES+) | |
|---|---|---|---|---|
| No. | Name | Yield | m/z | ¹H NMR; ¹⁹F NMR |
| 581 | 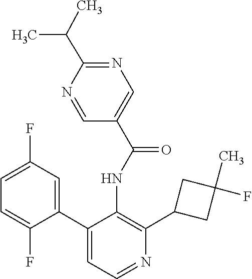 [SMILES: CC(C)c1ncc(C(=O)Nc2c(-c3cc(F)ccc3F)ccnc2C2CC(C)(F)C2)cn1] | 0.025 g 9% | 441.4 (M + 1) | (400 MHz, CD₃CN) δ 8.90 (s, 2H), 8.64 (d, J = 4.9 Hz, 1H), 8.57 (s, 1H), 7.31 (d, J = 4.9 Hz, 1H), 7.21-7.10 (m, 3H), 4.02 (quintet, J = 8.5 Hz, 1H), 3.22 (sept, J = 6.9 Hz, 1H), 2.58 (dqd, J = 19.8, 11.6, 10.2 Hz, 4H), 1.46 (d, J = 22.2 Hz, 3H), 1.31 (d, J = 6.9 Hz, 6H) |
| | N-(4-(2,5-difluorophenyl)-2-(3-fluoro-3- | | | |
| | methylcyclobutyl)pyridin-3-yl)-2- | | | |
| | isopropylpyrimidine-5-carboxamide | | | |
| | D1 | | | |
| 582 | 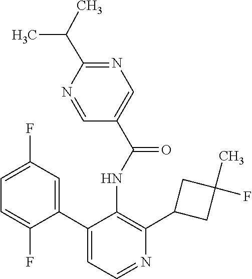 [SMILES: CC(C)c1ncc(C(=O)Nc2c(-c3cc(F)ccc3F)ccnc2C2CC(C)(F)C2)cn1] | 0.038 g 13% | 441.4 (M + 1) | (400 MHz, CD₃CN) δ 8.91 (s, 2H), 8.67 (d, J = 4.9 Hz, 1H), 8.57 (s, 1H), 7.31 (d, J = 4.8 Hz, 1H), 7.19-7.10 (m, 3H), 3.44-3.35 (m, 1H), 3.23 (sept, J = 6.9 Hz, 1H), 2.77- 2.66 (m, 2H), 2.46-2.39 (m, 2H), 1.51 (d, J = 22.2 Hz, 3H), 1.31 (d, J= 6.9 Hz, 6H) |
| | N-(4-(2,5-difluorophenyl)-2-(3-fluoro-3- | | | |
| | methylcyclobutyl)pyridin-3-yl)-2- | | | |
| | isopropylpyrimidine-5-carboxamide | | | |
| | D2 | | | |
| 583 | 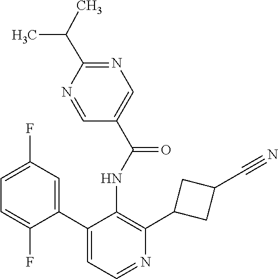 [SMILES: CC(C)c1ncc(C(=O)Nc2c(-c3cc(F)ccc3F)ccnc2C2CC(C#N)C2)cn1] | 0.039 g 23% | 434.3 (M + 1) | (400 MHz, DMSO-d₆, dr = 3:1) δ 10.32 (s, 4H), 8.98 (s, 2H), 8.97 (s, 6H), 8.71 (d, J = 4.9 Hz, 1H), 8.68 (d, J = 5.0 Hz, 3H),7.43 (d, J = 4.9 Hz, 4H), 7.35 (td, J = 9.1, 4.5 Hz, 4H), 7.26 (ddq, J = 20.6, 8.1, 4.0 Hz, 8H), 4.16 (quintet, J = 8.2 Hz, 3H), 3.94 (quintet, J = 8.9 Hz, 1H), 3.44-3.30 (m, 4H), 3.20 (dquintet, J = 13.7, 6.9 Hz, 4H), 2.73-2.55 (m, 16H), 1.28 (d, J = 6.9 Hz, 24H) |
| | N-(2-(3-cyanocyclobutyl)-4-(2,5- | | | |
| | difluorophenyl)pyridin-3-yl)-2- | | | |
| | isopropylpyrimidine-5-carboxamide | | | |
| | dr = 3:1 | | | |
| 584 | 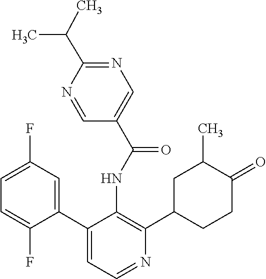 [SMILES: CC1CC(c2nccc(-c3cc(F)ccc3F)c2NC(=O)c2cnc(C(C)C)nc2)CCC1=O] | 0.063 g 21% | 465.4 (M + 1) | (400 MHz, DMSO-d₆) δ 10.44 (s, 1H), 8.97 (s, 2H), 8.65 (d, J = 4.9 Hz, 1H), 7.42-7.34 (m, 2H), 7.31-7.25 (m, 2H), 3.72-3.64 (m, 1H), 3.20 (sept, J = 6.9 Hz, 1H), 2.80-2.68 (m, 1H), 2.49-2.43 (m, 1H), 2.33-1.97 (m, 4H), 1.85-1.77 (m, 1H), 1.28 (d, J = 6.9 Hz, 6H), 1.01 (d, J = 6.1 Hz, 3H) |
| | N-(4-(2,5-difluorophenyl)-2-(3-methyl- | | | |
| | 4-oxocyclohexyl)pyridin-3-yl)-2- | | | |
| | isopropylpyrimidine-5-carboxamide | | | |
| | D1 | | | |
| 585 | 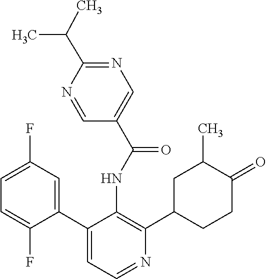 [SMILES: CC1CC(c2nccc(-c3cc(F)ccc3F)c2NC(=O)c2cnc(C(C)C)nc2)CCC1=O] | 0.101 g 34% | 465.4 (M + 1) | (400 MHz, DMSO-d₆) δ 10.44 (s, 1H), 9.00 (s, 2H), 8.62 (d, J = 4.9 Hz, 1H), 7.38 (dq, J = 10.1, 4.8 Hz, 2H), 7.32-7.24 (m, 2H), 3.68 (tt, J = 11.7, 3.4 Hz, 1H), 3.21 (sept, J = 6.9 Hz, 1H), 2.71- 2.56 (m, 2H), 2.28-2.25 (m, 1H), 2.13-1.96 (m, 3H), 1.84- 1.74 (m, 1H), 1.29 (d, J = 6.9 Hz, 6H), 0.92 (d, J = 6.5 Hz, 3H) |
| | N-(4-(2,5-difluorophenyl)-2-(3-methyl-4- | | | |
| | oxocyclohexyl)pyridin-3-yl)-2- | | | |
| | isopropylpyrimidine-5-carboxamide | | | |
| | D2 | | | |
| 586 | 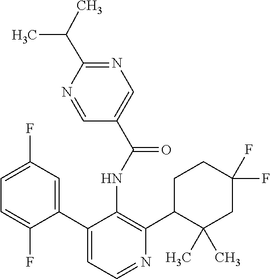 [SMILES: CC(C)c1ncc(C(=O)Nc2c(-c3cc(F)ccc3F)ccnc2C2CCC(F)(F)CC2(C)C)cn1] | 0.007 g 1% | 501.3 (M + 1) | (400 MHz, DMSO-d₆, rotamers are present) δ 10.56-10.22 (m, 1H), 8.93 (s, 2H), 8.63 (d, J = 4.9 Hz, 1H), 7.36-7.26 (m, 4H), 3.24-3.04 (m, 2H), 2.29-2.13 (m, 2H), 2.01-1.70 (m, 4H), 1.28 (d, J = 6.9 Hz, 6H), 0.92-0.74 (m, 6H) |
| | N-(2-(4,4-difluoro-2,2-dimethylcyclohexyl)- | | | |
| | 4-(2,5-difluorophenyl)pyridin-3-yl)-2- | | | |
| | isopropylpyrimidine-5-carboxamide | | | |
| 587 | 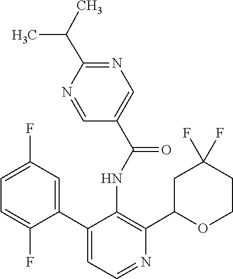 [SMILES: CC(C)c1ncc(C(=O)Nc2c(-c3cc(F)ccc3F)ccnc2C2CC(F)(F)CCO2)cn1] | 0.087 g 36% | 475.2 (M + 1) | (400 MHz, DMSO-d₆) δ 10.47 (s, 1H), 8.97 (s, 2H), 8.69 (d, J = 4.9 Hz, 1H), 7.56 (d, J = 4.9 Hz, 1H), 7.40-7.34 (m, 1H), 7.29 (tt, J = 7.6, 3.8 Hz, 2H), 4.89 (d, J = 11.2 Hz, 1H), 4.07-4.03 (m, 1H), 3.58 (td, J = 11.3, 3.9 Hz, 1H), 3.19 (sept, J = 6.9 Hz, 1H), 2.61-2.45 (m, 1H), 2.29- 2.22 (m, 1H), 2.16-2.00 (m, 2H), 1.28 (d, J = 6.9 Hz, 6H) |
| | N-(4-(2,5-difluorophenyl)-2-(4,4- | | | |
| | difluorotetrahydro-2H-pyran-2-yl)pyridin-3- | | | |
| | yl)-2-isopropylpyrimidine-5-carboxamide | | | |
| 588 | 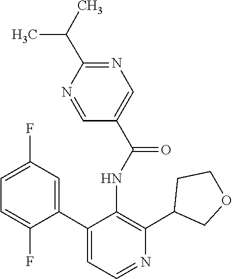 [SMILES: CC(C)c1ncc(C(=O)Nc2c(-c3cc(F)ccc3F)ccnc2C2CCOC2)cn1] | 0.093 g 42% | 425.2 (M + 1) | (400 MHz, DMSO-d₆) δ 10.39 (s, 1H), 8.97 (s, 2H), 8.64 (d, J = 4.9 Hz, 1H), 7.41-7.33 (m, 2H), 7.26 (dtt, J = 13.8, 6.9, 3.5 Hz, 2H), 4.03 (t, J = 7.3 Hz, 1H), 3.94- 3.71 (m, 4H), 3.19 (sept, J = 6.9 Hz, 1H), 2.25-2.17 (m, 2H), 1.28 (d, J = 6.9 Hz, 6H) |
| | N-(4-(2,5-difluorophenyl)-2- | | | |
| | (tetrahydrofuran-3-yl)pyridin-3-yl)-2- | | | |
| | isopropylpyrimidine-5-carboxamide | | | |
| 589 | 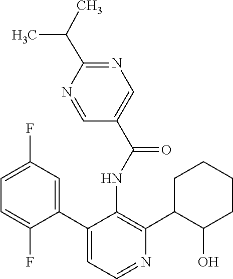 [SMILES: CC(C)c1ncc(C(=O)Nc2c(-c3cc(F)ccc3F)ccnc2C2CCCCC2O)cn1] | 0.013 g 11% | 453.4 (M + 1) | (400 MHz, DMSO-d₆) δ 10.32 (s, 1H), 8.97 (s, 2H), 8.62 (d, J = 4.9 Hz, 1H), 7.37-7.31 (m, 2H), 7.28-7.21 (m, 2H), 4.49 (s, 1H), 3.87- 3.80 (m, 1H), 3.19 (sept, J = 6.9 Hz, 1H), 2.94-2.88 (m, 1H), 1.95-1.92 (m, 1H), 1.79- 1.52 (m, 4H), 1.37-1.11 (m, 9H) |
| | N-(4-(2,5-difluorophenyl)-2-(2- | | | |
| | hydroxycyclohexyl)pyridin-3-yl)-2- | | | |
| | isopropylpyrimidine-5-carboxamide | | | |
| | P1 | | | |
| 590 | 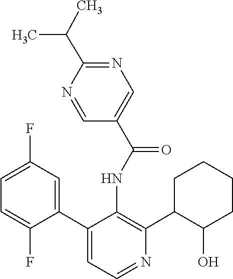 [SMILES: CC(C)c1ncc(C(=O)Nc2c(-c3cc(F)ccc3F)ccnc2C2CCCCC2O)cn1] | 0.079 g 12% | 453.4 (M + 1) | (400 MHz, DMSO-d₆) δ 10.43 (s, 1H), 8.94 (s, 2H), 8.61 (d, J = 5.0 Hz, 1H), 7.44 (d, J = 5.0 Hz, 1H), 7.39-7.33 (m, 1H), 7.27 (qd, J = 8.2, 3.8 Hz, 2H), 5.53 (s, 1H), 4.12 (s, 1H), 3.19 (tt, J = 14.4, 7.5 Hz, 2H), 1.96 (q, J = 12.0 Hz, 1H), 1.79-1.66 (m, 3H), 1.52-1.27 (m, 10H) |
| | N-(4-(2,5-difluorophenyl)-2-(2- | | | |
| | hydroxycyclohexyl)pyridin-3-yl)-2- | | | |
| | isopropylpyrimidine-5-carboxamide | | | |
| | P2 | | | |
| 591 | 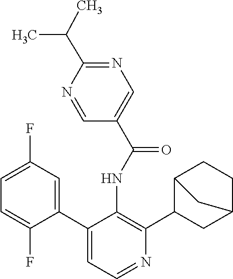 [SMILES: CC(C)c1ncc(C(=O)Nc2c(-c3cc(F)ccc3F)ccnc2C2CC3CCC2C3)cn1] | 0.047 g 20% | 449.4 (M + 1) | (400 MHz, DMSO-d₆) δ 10.36 (s, 1H), 8.96 (s, 2H), 8.59 (d, J = 4.9 Hz, 1H), 7.35 (quintet, J = 5.9 Hz, 2H), 7.26 (dtd, J = 9.6, 6.3, 3.4 Hz, 2H), 3.24-3.13 (m, 2H), 2.42-2.04 (m, 3H), 1.65 (d, J = 9.2 Hz, 1H), 1.48-1.43 (m, 3H), 1.28 (d, J = 6.9 Hz, 6H), 1.21-1.19 (m, 2H), 1.04 (d, J = 7.8 Hz, 1H) |
| | N-(2-(bicyclo[2.2.1]heptan-2-yl)-4-(2,5- | | | |
| | difluorophenyl)pyridin-3-yl)-2- | | | |
| | isopropylpyrimidine-5-carboxamide | | | |
| 592 | 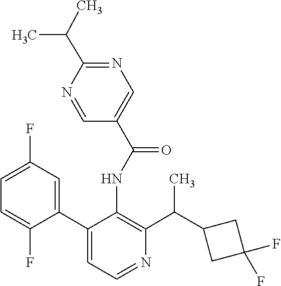 [SMILES: CC(C)c1ncc(C(=O)Nc2c(-c3cc(F)ccc3F)ccnc2C(C)C2CC(F)(F)C2)cn1] | 0.137 g 56% | 473.3 (M + 1) | (400 MHz, DMSO-d₆) δ 10.42 (d, J = 6.8 Hz, 1H), 8.96 (s, 2H), 8.64 (d, J = 4.9 Hz, 1H), 7.38-7.25 (m, 4H), 3.28-3.15 (m, 2H), 2.74-2.55 (m, 2H), 2.41-2.32 (m, 2H), 2.19-2.12 (m, 1H), 1.28 (d, J = 6.9 Hz, 6H), 1.15-1.14 (m, 3H) |
| | N-(2-(1-(3,3- | | | |
| | difluorocyclobutyl)ethyl)-4- | | | |
| | (2,5-difluorophenyl)pyridin-3-yl)-2- | | | |
| | isopropylpyrimidine-5-carboxamide | | | |
| 593 | 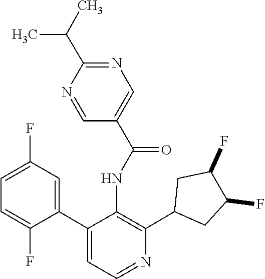 [SMILES: CC(C)c1ncc(C(=O)Nc2c(-c3cc(F)ccc3F)ccnc2C2C[C@@H](F)[C@@H](F)C2)cn1] | 0.072 g 31% | 459.2 (M + 1) | (400 MHz, DMSO-d₆) δ 10.40 (s, 1H), 8.97 (s, 2H), 8.65 (d, J = 4.9 Hz, 1H), 7.41 (d, J = 4.9 Hz, 1H), 7.36 (td, J = 9.2, 4.5 Hz, 1H), 7.30- 7.22 (m, 2H), 5.35-5.18 (m, 2H), 4.01 (quintet, J = 7.9 Hz, 1H), 3.19 (sept, J = 6.9 Hz, 1H), 2.33-2.11 (m, 4H), 1.28 (d, J = 6.9 Hz, 6H) |
| | N-(2-(anti-3,4-difluorocyclopentyl)- | | | |
| | 4-(2,5-difluorophenyl)pyridin-3- | | | |
| | yl)-2-isopropylpyrimidine-5-carboxamide | | | |
| 594 | 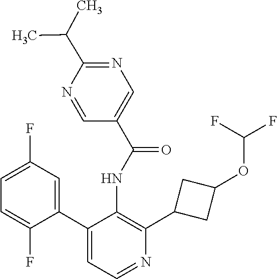 [SMILES: CC(C)c1ncc(C(=O)Nc2c(-c3cc(F)ccc3F)ccnc2C2CC(OC(F)F)C2)cn1] | 0.044 g 24% | 475.2 (M + 1) | (400 MHz, DMSO-d₆) δ 10.34 (s, 1H), 8.99 (s, 2H), 8.69 (d, J = 4.9 Hz, 1H), 7.42-7.34 (m, 2H), 7.26 (dddd, J = 18.0, 10.5, 7.1, 3.4 Hz, 2H), 6.64 (t, J = 75.9 Hz, 1H), 4.62 (quintet, J = 7.5 Hz, 1H), 3.54-3.46 (m, 1H), 3.20 (sept, J = 6.9 Hz, 1H), 2.63-2.56 (m, 2H), 2.46- 2.41 (m, 2H), 1.29 (d, J = 6.9 Hz, 6H); (376 MHz, DMSO-d₆) δ-8.15, -118.8 (d, J = 18 Hz), -120.4 (d, J = 18 Hz) |
| | N-(2-(3-(difluoromethoxy)cyclobutyl)- | | | |
| | 4-(2,5-difluorophenyl)pyridin-3- | | | |
| | yl)-2-isopropylpyrimidine-5-carboxamide | | | |
| | D1 | | | |
| 595 | 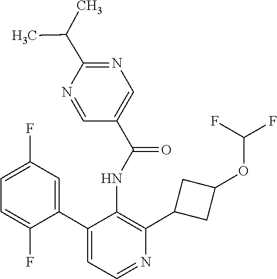 [SMILES: CC(C)c1ncc(C(=O)Nc2c(-c3cc(F)ccc3F)ccnc2C2CC(OC(F)F)C2)cn1] | 0.044 g 24% | 475.2 (M + 1) | (400 MHz, DMSO-d₆) δ 10.34 (s, 1H), 8.99 (s, 2H), 8.69 (d, J = 4.9 Hz, 1H), 7.42-7.34 (m, 2H), 7.26 (dddd, J = 18.0, 10.5, 7.1, 3.4 Hz, 2H), 6.64 (t, J = 75.9 Hz, 1H), 4.62 (quintet, J = 7.5 Hz, 1H), 3.54-3.46 (m, 1H), 3.20 (sept, J = 6.9 Hz, 1H), 2.63-2.56 (m, 2H), 2.46- 2.41 (m, 2H), 1.29 (d, J = 6.9 Hz, 6H); (376 MHz, DMSO-d₆) δ-81.5, -118.8 (d, J = 18 Hz), -120.4 (d, J = 18 Hz) |
| | N-(2-(3-(difluoromethoxy)cyclobutyl)- | | | |
| | 4-(2,5-difluorophenyl)pyridin-3-yl)-2- | | | |
| | isopropylpyrimidine-5-carboxamide | | | |
| | D2 | | | |
| 596 | 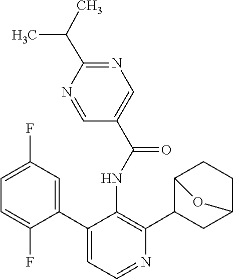 [SMILES: CC(C)c1ncc(C(=O)Nc2c(-c3cc(F)ccc3F)ccnc2C2CC3CCC2O3)cn1] | 0.113 g 49% | 451.2 (M + 1) | (400 MHz, DMSO-d₆) δ 10.36 (s, 1H), 8.96 (s, 2H), 8.59 (d, J = 4.9 Hz, 1H), 7.39-7.34 (m, 2H), 7.31-7.22 (m, 2H), 4.68-4.52 (m, 2H), 3.42-3.35 (m, 1H), 3.20 (sept, J = 6.9 Hz, 1H), 2.44- 2.37 (m, 1H), 1.73 (dd, J = 11.4, 8.9 Hz, 1H), 1.55-1.46 (m, 4H), 1.28 (d, J = 6.9 Hz, 6H) |
| | N-(2-(7-oxabicyclo[2.2.1]heptan-2- | | | |
| | yl)-4-(2,5-difluorophenyl)pyridin-3- | | | |
| | yl)-2-isopropylpyrimidine-5-carboxamide | | | |
| 597 | 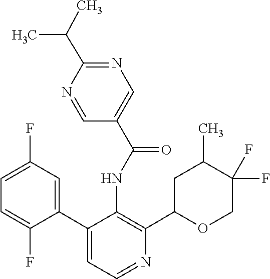 [SMILES: CC(C)c1ncc(C(=O)Nc2c(-c3cc(F)ccc3F)ccnc2C2CC(C)C(F)(F)CO2)cn1] | 0.130 g 41% | 489.4 (M + 1) | (400 MHz, DMSO-d₆) δ 10.36 (s, 1H), 8.96 (s, 2H), 8.68 (d, J = 4.9 Hz, 1H), 7.54 (d, J = 4.9 Hz, 1H), 7.37 (td, J = 9.6, 4.6 Hz, 1H), 7.33-7.27 (m, 2H), 5.20 (d, J = 5.1 Hz, 1H), 3.85 (ddd, J = 21.7, 12.5, 4.3 Hz, 1H), 3.66 (td, J = 12.5, 7.6 Hz, 1H), 3.19 (sept, J = 6.9 Hz, 1H), 2.68-2.54 (m, 1H), 2.43 (t, J = 6.7 Hz, 1H), 1.82 (ddt, J = 13.7, 6.3, 3.3 Hz, 1H), 1.28 (d, J = 6.9 Hz, 6H), 1.12 (d, J = 7.0 Hz, 3H); (376 MHz, DMSO-d₆) δ-104.3 (d, J = 243 Hz), -116.1 (d, J = 241 Hz), -118.8 (d, J = 18 Hz), -120.5 (d, J = 18 Hz) |
| | N-(2-(5,5-difluoro-4-methyltetrahydro- | | | |
| | 2H-pyran-2-yl)-4-(2,5- | | | |
| | difluorophenyl)pyridin-3- | | | |
| | yl)-2-isopropylpyrimidine-5-carboxamide | | | |
| | D1 | | | |
| 598 | 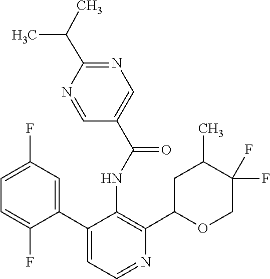 [SMILES: CC(C)c1ncc(C(=O)Nc2c(-c3cc(F)ccc3F)ccnc2C2CC(C)C(F)(F)CO2)cn1] | 0.048 g 15% | 489.4 (M + 1) | (400 MHz, DMSO-d₆) δ 10.39 (s, 1H), 8.97 (s, 2H), 8.67 (d, J = 4.9 Hz, 1H), 7.52 (d, J = 4.8 Hz, 1H), 7.36 (td, J = 9.2, 4.4 Hz, 1H), 7.29 (tt, J = 7.7, 3.8 Hz, 2H), 4.97 (d, J = 10.8 Hz, 1H), 3.93 (t, J = 11.8 Hz, 1H), 3.76 (dd, J = 32.5, 12.1 Hz, 1H), 3.19 (sept, J = 6.9 Hz, 1H), 2.37 (dtt, J = 26.2, 12.7, 6.4 Hz, 1H), 2.07 (q, J = 12.2 Hz, 1H), 1.92 (d, J = 14.5 Hz, 1H), 1.28 (d, J = 6.9 Hz, 6H), 1.04 (d, J = 6.6 Hz, 3H); (376 MHz, DMSO-d₆) δ-104.3 (d, J = 243 Hz), -116.1 (d, J = 241 Hz), -118.8 (d, J = 18 Hz), -120.5 (d, J = 18 Hz) |
| | N-(2-(5,5-difluoro-4-methyltetrahydro- | | | |
| | 2H-pyran-2-yl)-4-(2,5-difluorophenyl) | | | |
| | pyridin-3-yl)-2-isopropylpyrimidine- | | | |
| | 5-carboxamide | | | |
| | D2 | | | |
| 599 | 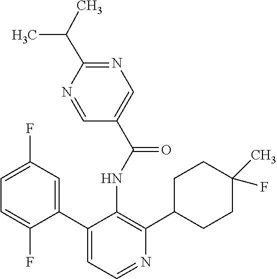 [SMILES: CC(C)c1ncc(C(=O)Nc2c(-c3cc(F)ccc3F)ccnc2C2CCC(C)(F)CC2)cn1] | 0.041 g 20% | 469.3 (M + 1) | (400 MHz, DMSO-d₆) δ 10.38 (s, 1H), 8.95 (s, 2H), 8.62 (d, J = 4.9 Hz, 1H), 7.38-7.32 (m, 2H), 7.29-7.22 (m, 2H), 3.19 (sept, J = 6.9 Hz, 1H), 3.03 (t, J = 11.9 Hz, 1H), 1.96-1.84 (m, 4H), 1.61- 1.47 (m, 4H), 1.35-1.21 (m, 9H); (376 MHz, DMSO-d₆) δ-118.9 (d, J = 18 Hz), -120.3 (d, J = 18 Hz), -149.8 |
| | N-(4-(2,5-difluorophenyl)-2-(4-fluoro-4- | | | |
| | methylcyclohexyl)pyridin-3-yl)-2- | | | |
| | isopropylpyrimidine-5-carboxamide | | | |
| | D1 | | | |
| 600 | 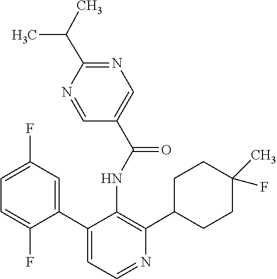 [SMILES: CC(C)c1ncc(C(=O)Nc2c(-c3cc(F)ccc3F)ccnc2C2CCC(C)(F)CC2)cn1] | 0.042 g 20% | 469.3 (M + 1) | (400 MHz, DMSO-d₆) δ 10.37 (s, 1H), 8.96 (s, 2H), 8.62 (d, J = 4.9 Hz, 1H), 7.38-7.32 (m, 2H), 7.26 (dtd, J = 11.2, 7.7, 3.4 Hz, 2H), 3.24-3.07 (m, 2H), 1.88-1.72 (m, 8H), 1.43 (d, J = 22.9 Hz, 3H), 1.28 (d, J = 6.9 Hz, 6H); (376 MHz, DMSO-d₆) δ-118.9 (d, J = 18 Hz), -120.3 (d, J = 18 Hz), -125.1 |
| | N-(4-(2,5-difluorophenyl)-2-(4-fluoro-4- | | | |
| | methylcyclohexyl)pyridin-3-yl)-2- | | | |
| | isopropylpyrimidine-5-carboxamide | | | |
| | D2 | | | |
| 601 | 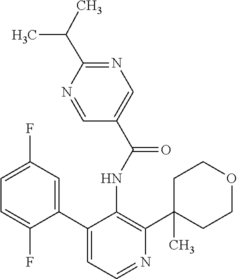 [SMILES: CC(C)c1ncc(C(=O)Nc2c(-c3cc(F)ccc3F)ccnc2C2(C)CCOCC2)cn1] | 0.006 g 0.8% | 453.2 (M + 1) | (400 MHz, DMSO-d₆, rotamers are present) δ 10.41 (s, 0.5H), 10.23 (s, 0.5H), 9.00 (s, 1H), 8.83 (s, 1H), 8.73 (s, 0.5H), 8.66 (d, J = 4.8 Hz, 0.5H), 7.50 (s, 0.5H), 7.42-7.24 (m, 3.5H), 3.73-3.60 (m, 2H), 3.56-3.46 (m, 2H), 3.24-3.15 (m, 1H), 2.45-2.42 (m, 0.5H), 2.35- 2.25 (m, 1.5H), 1.81-1.66 (m, 2H), 1.35 (s, 1.5H), 1.31 (s, 1.5H), 1.28 (dd, J = 6.9, 5.5 Hz, 6H) |
| | N-(4-(2,5-difluorophenyl)-2-(4- | | | |
| | methyltetrahydro-2H-pyran-4-yl)pyridin- | | | |
| | 3-yl)-2-isopropylpyrimidine-5-carboxamide | | | |
| 602 | 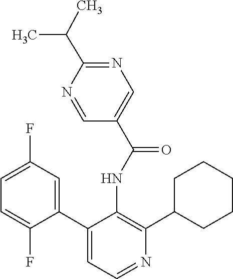 [SMILES: CC(C)c1ncc(C(=O)Nc2c(-c3cc(F)ccc3F)ccnc2C2CCCCC2)cn1] | 0.029 g 34% | 437.4 (M + 1) | (400 MHz, DMSO-d₆) δ 10.36 (s, 1H), 8.95 (s, 2H), 8.61 (d, J = 4.9 Hz, 1H), 7.35 (dq, J = 10.1, 4.8 Hz, 2H), 7.25 (tdd, J = 10.9, 7.5, 3.3 Hz, 2H), 3.19 (sept, J = 6.9 Hz, 1H), 3.01 (tt, J = 11.5, 3.1 Hz, 1H), 1.78-1.70 (m, 4H), 1.70-1.55 (m, 3H), 1.34- 1.17 (m, 9H) |
| | N-(2-cyclohexyl-4-(2,5- | | | |
| | difluorophenyl)pyridin-3-yl)-2- | | | |
| | isopropylpyrimidine-5-carboxamide | | | |
| 603 | 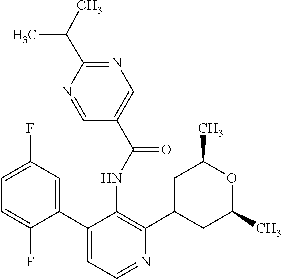 [SMILES: CC(C)c1ncc(C(=O)Nc2c(-c3cc(F)ccc3F)ccnc2C2C[C@@H](C)O[C@@H](C)C2)cn1] | 0.115 g 30% | 467.2 (M + 1) | (400 MHz, CD₃OD) δ 8.95 (s, 2H), 8.62 (d, J = 5.0 Hz, 1H), 7.35 (d, J = 5.0 Hz, 1H), 7.24-7.17 (m, 1H), 7.17-7.11 (m, 2H), 3.63-3.56 (m, 2H), 3.36 (tt, J = 11.8, 3.9 Hz, 1H), 3.22 (sept, J = 6.9 Hz, 1H), 1.75 (dt, J = 12.5, 1.8 Hz, 2H), 1.63 (q, J = 12.1 Hz, 2H), 1.33 (d, J = 6.9 Hz, 6H), 1.20 (d, J = 6.2 Hz, 6H) |
| | N-(4-(2,5-difluorophenyl)-2-((2R,6S)-2,6- | | | |
| | dimethyltetrahydro-2H-pyran-4-yl)pyridin-3- | | | |
| | yl)-2-isopropylpyrimidine-5-carboxamide | | | |
| | D1 | | | |
| 604 | 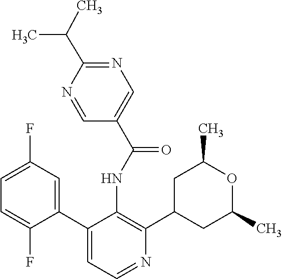 [SMILES: CC(C)c1ncc(C(=O)Nc2c(-c3cc(F)ccc3F)ccnc2C2C[C@@H](C)O[C@@H](C)C2)cn1] | 0.027 g 7% | 467.4 (M + 1) | (400 MHz, CD₃OD) δ 8.95 (s, 2H), 8.66 (d, J = 4.9 Hz, 1H), 7.36 (d, J = 4.9 Hz, 1H), 7.25-7.13 (m, 3H), 4.31-4.23 (m, 2H), 3.71 (t, J = 6.1 Hz, 1H), 3.25 (sept, J = 6.9 Hz, 1H), 1.95 (d, J = 9.7 Hz, 2H), 1.64 (ddd, J = 13.4, 11.3, 6.2 Hz, 2H), 1.36 (d, J = 6.9 Hz, 6H), 1.15 (d, J = 6.2 Hz, 6H) |
| | N-(4-(2,5-difluorophenyl)-2-((2R,6S)-2,6- | | | |
| | dimethyltetrahydro-2H-pyran-4-yl)pyridin-3- | | | |
| | yl)-2-isopropylpyrimidine-5-carboxamide | | | |
| | D2 | | | |
| 605 | 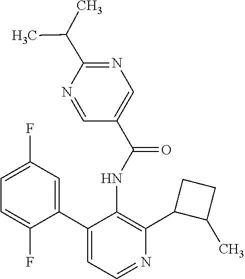 [SMILES: CC(C)c1ncc(C(=O)Nc2c(-c3cc(F)ccc3F)ccnc2C2CCC2C)cn1] | 0.143 g 44% | 423.2 (M + 1) | (400 MHz, DMSO-d₆) δ 10.31 (s, 1H), 8.97 (s, 2H), 8.65 (d, J = 4.9 Hz, 1H), 7.38-7.32 (m, 2H), 7.29-7.21 (m, 2H), 3.45 (q, J = 8.6 Hz, 1H), 3.19 (sept, J = 6.9 Hz, 1H), 2.74-2.64 (m, 1H), 2.22- 1.94 (m, 3H), 1.53 (quintet, J = 9.5 Hz, 1H), 1.28 (d, J = 6.9 Hz, 6H), 1.03-1.01 (m, 3H) |
| | N-(4-(2,5-difluorophenyl)-2-(2- | | | |
| | methylcyclobutyl)pyridin-3-yl)-2- | | | |
| | isopropylpyrimidine-5-carboxamide | | | |
| 606 | 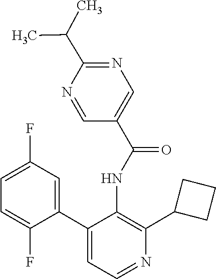 [SMILES: CC(C)c1ncc(C(=O)Nc2c(-c3cc(F)ccc3F)ccnc2C2CCC2)cn1] | 0.055 g 35% | 409.2 (M + 1) | (400 MHz, DMSO-d₆) δ 10.27 (s, 1H), 8.97 (s, 2H), 8.65 (d, J = 4.9 Hz, 1H), 7.35 (dq, J = 9.3, 4.6 Hz, 2H), 7.25 (dddd, J = 17.3, 10.4, 6.8, 3.5 Hz, 2H), 3.92 (quintet, J = 8.6 Hz, 1H), 3.19 (sept, J = 6.9 Hz, 1H), 2.38-2.32 (m, 2H), 2.21-2.13 (m, 2H), 1.95 (dq, J = 18.7, 9.3 Hz, 1H), 1.82-1.74 (m, 1H), 1.28 (d, J = 6.9 Hz, 6H) |
| | N-(2-cyclobutyl-4-(2,5- | | | |
| | difluorophenyl)pyridin-3- | | | |
| | yl)-2-isopropylpyrimidine-5-carboxamide | | | |
| 607 | 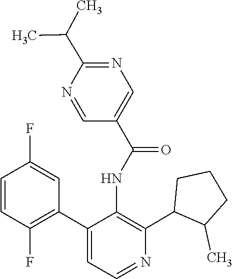 [SMILES: CC(C)c1ncc(C(=O)Nc2c(-c3cc(F)ccc3F)ccnc2C2CCCC2C)cn1] | 0.083 g 37% | 437.4 (M + 1) | (400 MHz, DMSO-d₆) δ 10.37 (s, 1H), 8.95 (s, 2H), 8.63 (d, J = 4.9 Hz, 1H), 7.35 (dt, J = 13.9, 4.7 Hz, 2H), 7.25 (td, J = 6.4, 3.5 Hz, 2H), 3.19 (sept, J = 6.9 Hz, 1H), 3.04 (q, J = 8.7 Hz, 1H), 2.28 (s, 1H), 2.00-1.67 (m, 5H), 1.29-1.19 (m, 7H), 0.84 (s, 3H) |
| | N-(4-(2,5-difluorophenyl)-2-(2- | | | |
| | methylcyclopentyl)pyridin-3-yl)-2- | | | |
| | isopropylpyrimidine-5-carboxamide | | | |
| 608 | 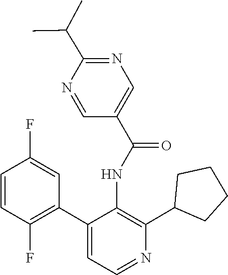 [SMILES: CC(C)c1ncc(C(=O)Nc2c(-c3cc(F)ccc3F)ccnc2C2CCCC2)cn1] | 0.037 g 34% | 423.2 (M + 1) | (400 MHz, DMSO-d₆) δ 10.34 (s, 1H), 8.96 (s, 2H), 8.61 (d, J = 4.9 Hz, 1H), 7.38-7.31 (m, 2H), 7.25 (tdd, J = 10.0, 6.6, 3.3 Hz, 2H), 3.49 (dt, J = 16.1, 8.0 Hz, 1H), 3.19 (sept, J = 13.8, 6.9 Hz, 1H), 1.94-1.72 (m, 6H), 1.59 (t, J = 5.7 Hz, 2H), 1.28 (d, J = 6.9 Hz, 6H) |
| | N-(2-cyclopentyl-4-(2,5- | | | |
| | difluorophenyl)pyridin-3- | | | |
| | yl)-2-isopropylpyrimidine-5-carboxamide | | | |
| 609 | 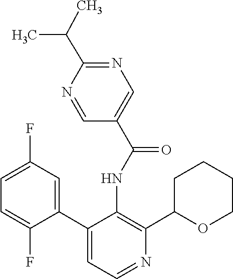 [SMILES: CC(C)c1ncc(C(=O)Nc2c(-c3cc(F)ccc3F)ccnc2C2CCCCO2)cn1] | 0.101 g 45% | 439.2 (M + 1) | (400 MHz, DMSO-d₆) δ 10.36 (s, 1H), 8.98 (s, 2H), 8.64 (d, J = 4.9 Hz, 1H), 7.47 (d, J = 4.8 Hz, 1H), 7.35 (td, J = 9.6, 4.6 Hz, 1H), 7.27 (ddd, J = 8.4, 5.9, 2.9 Hz, 2H), 4.67 (dd, J = 10.8, 1.9 Hz, 1H), 3.95-3.92 (m, 1H), 3.51-3.45 (m, 1H), 3.19 (sept, J = 6.9 Hz, 1H), 1.99- 1.84 (m, 2H), 1.71 (d, J = 13.4 Hz, 1H), 1.62-1.49 (m, 3H), 1.28 (d, J = 6.9 Hz, 6H) |
| | N-(4-(2,5-difluorophenyl)-2- | | | |
| | (tetrahydro-2H-pyran-2-yl)pyridin-3-yl)- | | | |
| | 2-isopropylpyrimidine-5-carboxamide | | | |
| 610 | 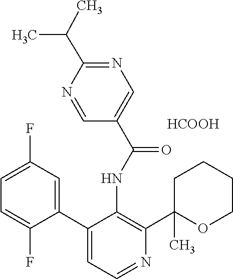 [SMILES: CC(C)c1ncc(C(=O)Nc2c(-c3cc(F)ccc3F)ccnc2C2(C)CCCCO2)cn1 ; "HCOOH"] | 0.005 g 0.3% | 453.2 (M + 1) | (400 MHz, DMSO-d₆) δ 10.46 (s, 1H), 9.00 (s, 2H), 8.73 (s, 1H), 8.49 (s, 0.3H), 7.54 (s, 1H), 7.42-7.27 (m, 3H), 3.75 (dt, J = 11.3, 4.1 Hz, 1H), 3.51-3.44 (m, 1H), 3.20 (sept, J = 6.9 Hz, 1H), 1.69-1.61 (m, 2H), 1.57-1.49 (m, 1H), 1.45-1.33 (m, 5H), 1.28 (d, J = 6.9 Hz, 6H), 1.23 (s, 1H) |
| | N-(4-(2,5-difluorophenyl)-2-(2- | | | |
| | methyltetrahydro-2H-pyran-2- | | | |
| | yl)pyridin-3-yl)-2-isopropylpyrimidine- | | | |
| | 5-carboxamide formic acid salt | | | |
| 611 | 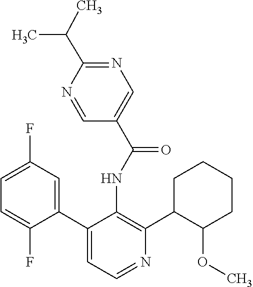 [SMILES: COC1CCCCC1c1nccc(-c2cc(F)ccc2F)c1NC(=O)c1cnc(C(C)C)nc1] | 0.088 g 23% | 467.4 (M + 1) | (400 MHz, DMSO-d₆) δ 10.37 (s, 1H), 8.95 (s, 2H), 8.63 (d, J = 4.9 Hz, 1H), 7.34 (dq, J = 9.5, 4.8 Hz, 2H), 7.27-7.23 (m, 2H), 3.73 (td, J = 10.3, 3.8 Hz, 1H), 3.18 (sept, J = 6.9 Hz, 1H), 3.07- 3.00 (m, 4H), 2.22 (d, J = 9.0 Hz, 1H), 1.84 (d, J = 12.8 Hz, 1H), 1.76 (d, J = 11.7 Hz, 1H), 1.63 (t, J = 6.4 Hz, 1H), 1.49-1.39 (m, 1H), 1.36- 1.08 (m, 9H) |
| | N-(4-(2,5-difluorophenyl)-2-(2- | | | |
| | methoxycyclohexyl)pyridin-3-yl)-2- | | | |
| | isopropylpyrimidine-5-carboxamide | | | |
| 612 | 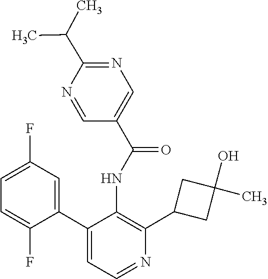 [SMILES: CC(C)c1ncc(C(=O)Nc2c(-c3cc(F)ccc3F)ccnc2C2CC(C)(O)C2)cn1] | 0.090 g 32% | 439.2 (M + 1) | (400 MHz, DMSO-d₆, dr = 1:1) δ 10.27 (s, 1H), 10.26 (s, 1H), 8.98 (d, J = 2.6 Hz, 2H), 8.97 (s, 2H), 8.63 (dd, J = 4.9, 2.6 Hz, 2H), 7.38-7.32 (m, 4H), 7.30-7.20 (m, 4H), 4.96 (s, 1H), 4.85 (s, 1H), 3.95 (quintet, J = 8.6 Hz, 1H), 3.42-3.37 (m, 1H), 3.20 (dtd, J = 13.8, 6.9, 1.7 Hz, 2H), 2.40-2.30 (m, 4H), 2.24- 2.17 (m, 4H), 1.28 (d, J = 6.9 Hz, 15H), 1.20 (s, 3H) |
| | N-(4-(2,5-difluorophenyl)-2-(3-hydroxy-3- | | | |
| | methylcyclobutyl)pyridin-3-yl)-2- | | | |
| | isopropylpyrimidine-5-carboxamide | | | |
| | dr = 1:1 | | | |

### Example 613

### Synthesis of N-(4-(2,5-difluorophenyl)-2-(2-fluorocyclohexyl)pyridin-3-yl)-2-isopropylpyrimidine-5-carboxamide

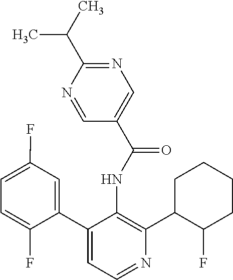 [SMILES: CC(C)c1ncc(C(=O)Nc2c(-c3cc(F)ccc3F)ccnc2C2CCCCC2F)cn1]

### Step 1. Preparation of tert-butyl (4-(2,5-difluorophenyl)-2-(2-hydroxycyclohexyl)pyridin-3-yl)carbamate

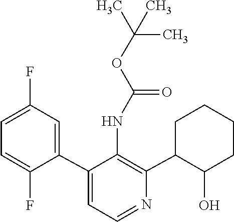 [SMILES: CC(C)(C)OC(=O)Nc1c(-c2cc(F)ccc2F)ccnc1C1CCCCC1O]

To a mixture of tert-butyl (2-chloro-4-(2,5-difluorophenyl)pyridin-3-yl)carbamate (1.20 g, 3.52 mmol), cesium carbonate (1.21 g, 3.70 mmol), nickel(II) chloride dimethoxyethane adduct (0.039 g, 0.18 mmol), 4-tert-butyl-2-(4-tert-butyl-2-pyridyl)pyridine (0.071 g, 0.26 mmol), (4,4″-di-tert-butyl-2,2″-bipyridine)bis[3,5-difluoro-2-[5-trifluoromethyl-2-pyridinyl-kN)phenyl-kC]iridium(III) hexafluorophosphate (0.020 g, 0.018 mmol), and 2-hydroxycyclohexane-1-carboxylic acid (0.508 g, 3.52 mmol), split into two vials, was added anhydrous N,N-dimethylacetamide (22 mL) to each, and the headspace was flushed with nitrogen for 10 seconds. The vials were sealed and placed in front of 4 Kessil PR160L lights (440 nm) for 16 h. The two reaction mixtures were combined and diluted with ethyl acetate (300 mL). The organic phase was washed with saturated sodium bicarbonate (100 mL), saturated ammonium chloride (3×100 mL), and brine (100 mL), then dried over anhydrous magnesium sulfate, filtered, and concentrated in vacuo. The residue was purified by column chromatography, using a gradient of 10 to 100% of ethyl acetate in heptane as eluent, to afford the title compound as a yellow solid (0.535 g, 75% yield): MS (ES+) m/z 405.4 (M+1).

### Step 2. Preparation of 2-(3-amino-4-(2,5-difluorophenyl)pyridin-2-yl)cyclohexan-1-ol

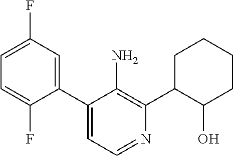 [SMILES: Nc1c(-c2cc(F)ccc2F)ccnc1C1CCCCC1O]

To a mixture of tert-butyl (4-(2,5-difluorophenyl)-2-(2-hydroxycyclohexyl)pyridin-3-yl)carbamate (0.535 g, 1.32 mmol) in anhydrous dichloromethane (4.4 mL) was added 4.0 M hydrochloric acid in anhydrous dioxane (3.3 mL, 13 mmol). The reaction mixture was stirred at ambient temperature for 3.5 h. The reaction mixture was diluted with saturated potassium carbonate (50 mL) and the aqueous phase was extracted with ethyl acetate (2×75 mL). The combined organic phases were washed with brine (50 mL), dried over anhydrous magnesium sulfate, filtered, and concentrated in vacuo. The residue was purified by column chromatography, using a gradient of 15 to 37% of ethyl acetate in heptane as eluent, to afford the title compound as a colorless solid (0.249 g, 62% yield): ¹H NMR (400 MHz, CDCl₃) δ 8.03 (dd, J=4.9, 1.4 Hz, 1H), 7.16 (td, J=8.8, 4.5 Hz, 1H), 7.07 (tdd, J=11.0, 7.5, 3.4 Hz, 2H), 6.88 (d, J=4.9 Hz, 1H), 4.14-4.07 (m, 1H), 3.85 (s, 2H), 2.92 (s, 1H), 2.72 (ddd, J=12.2, 9.3, 3.1 Hz, 1H), 2.12-2.09 (m, 1H), 1.96 (d, J=13.6 Hz, 1H), 1.86-1.77 (m, 2H), 1.61-1.42 (m, 3H), 1.39-1.28 (m, 1H); MS (ES+) m/z 305.4 (M+1).

### Step 3. Preparation of 4-(2,5-difluorophenyl)-2-(2-fluorocyclohexyl)pyridin-3-amine

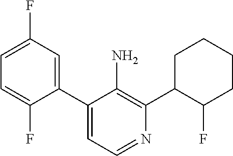 [SMILES: Nc1c(-c2cc(F)ccc2F)ccnc1C1CCCCC1F]

To a mixture of 2-(3-amino-4-(2,5-difluorophenyl)pyridin-2-yl)cyclohexan-1-ol (0.100 g, 0.329 mmol) in anhydrous dichloromethane (3.3 mL) at 0° C. was added diethylaminosulfur trifluoride (0.21 mL, 1.6 mmol) and the reaction mixture was left to stir at 0° C. for 1 h. To the reaction mixture was added water (20 mL), and the aqueous phase was extracted with ethyl acetate (60 mL). The organic phase was washed with brine (20 mL), dried over anhydrous magnesium sulfate, filtered, and concentrated in vacuo. The residue was purified by column chromatography, eluting with a gradient of 5 to 100% of ethyl acetate in heptane to afford the title compound as a yellow solid (0.058 g, 58% yield): MS (ES+) m/z 307.4 (M+1).

### Step 4. Preparation of N-(4-(2,5-difluorophenyl)-2-(2-fluorocyclohexyl)pyridin-3-yl)-2-isopropylpyrimidine-5-carboxamide

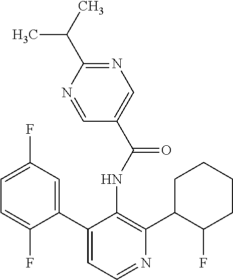 [SMILES: CC(C)c1ncc(C(=O)Nc2c(-c3cc(F)ccc3F)ccnc2C2CCCCC2F)cn1]

To a mixture of 4-(2,5-difluorophenyl)-2-(2-fluorocyclohexyl)pyridin-3-amine (0.058 g, 0.19 mmol) in anhydrous tetrahydrofuran (3.8 mL) was added N,N-diisopropylethylamine (0.33 mL, 1.9 mmol), 2-chloro-1-methylpyridinium iodide (0.146 g, 0.573 mmol) and 2-isopropylpyrimidine-5-carboxylic acid (0.035 g, 0.21 mmol). The reaction mixture was stirred at 40° C. for 24 h. After cooling to ambient temperature, to the reaction mixture was added 2-chloro-1-methylpyridinium iodide (0.049 g, 0.19 mmol) and 2-isopropylpyrimidine-5-carboxylic acid (0.016 g, 0.095 mmol). The reaction mixture was stirred at 40° C. for 18 h. After cooling to ambient temperature, the reaction mixture was diluted with 1 M sodium hydroxide and the aqueous phase was extracted with ethyl acetate (150 mL). The organic phase was washed with saturated ammonium chloride (50 mL) and brine (50 mL), dried over anhydrous magnesium sulfate, filtered, and concentrated in vacuo. The residue was purified by column chromatography, eluting with a gradient of 15 to 75% of ethyl acetate in heptane, to afford the title compound as a colorless solid (0.034 g, 21% yield): ¹H NMR (400 MHz, DMSO-d₆) δ 10.41 (s, 1H), 8.96 (s, 2H), 8.66 (d, J=4.9 Hz, 1H), 7.40-7.33 (m, 2H), 7.27 (tq, J=8.3, 4.1 Hz, 2H), 5.06 (dtd, J=48.5, 10.3, 4.5 Hz, 1H), 3.27-3.14 (m, 2H), 2.16-2.12 (m, 1H), 1.93-1.89 (m, 1H), 1.78 (d, J=11.4 Hz, 1H), 1.63 (d, J=11.1 Hz, 1H), 1.58-1.36 (m, 3H), 1.29-1.18 (m, 7H); ¹H NMR (400 MHz, DMSO-d₆) δ −118.9, −120.3, −169.4; MS (ES+) m/z 455.2 (M+1).

### Example 614

### Synthesis of N-(4-(2,5-difluorophenyl)-2-((6R)-6-methylmorpholin-2-yl)pyridin-3-yl)-2-isopropylpyrimidine-5-carboxamide hydrochloric formic acid mixed salt

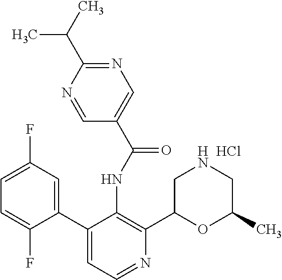 [SMILES: CC(C)c1ncc(C(=O)Nc2c(-c3cc(F)ccc3F)ccnc2C2CNC[C@@H](C)O2)cn1 ; Cl]

### Step 1. Preparation of tert-butyl (6R)-2-(4-(2,5-difluorophenyl)-3-(2-isopropylpyrimidine-5-carboxamido)pyridin-2-yl)-6-methylmorpholine-4-carboxylate

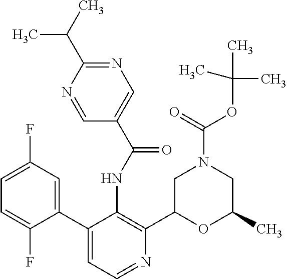 [SMILES: CC(C)c1ncc(C(=O)Nc2c(-c3cc(F)ccc3F)ccnc2C2CN(C(=O)OC(C)(C)C)C[C@@H](C)O2)cn1]

To a vial containing N-(2-chloro-4-(2,5-difluorophenyl)pyridin-3-yl)-2-isopropylpyrimidine-5-carboxamide (0.400 g, 1.03 mmol), cesium carbonate (0.536 g, 1.65 mmol), nickel(II) chloride dimethoxyethane adduct (0.023 g, 0.10 mmol), 4-tert-butyl-2-(4-tert-butyl-2-pyridyl)pyridine (0.041 g, 0.15 mmol), (4,4″-di-tert-butyl-2,2″-bipyridine)bis[3,5-difluoro-2-[5-trifluoromethyl-2-pyridinyl-kN)phenyl-kC]iridium(III) hexafluorophosphate (0.012 g, 0.010 mmol), and (2R,6R)-4-(tert-butoxycarbonyl)-6-methylmorpholine-2-carboxylic acid (0.379 g, 1.54 mmol) was added anhydrous N,N-dimethylacetamide (26.0 mL) and the headspace was flushed with nitrogen for 10 seconds. The vial was sealed and placed in front of 4 Kessil PR160L lights (440 nm) for 16 h. The reaction mixture was diluted with ethyl acetate (150 mL) and the organic phase was washed with saturated sodium bicarbonate (50 mL), water (3×50 mL), brine (50 mL), dried over anhydrous magnesium sulfate, filtered, and concentrated in vacuo. The residue was purified by column chromatography, using a gradient of ethyl acetate in heptane as eluent, to afford the title compound as a colorless solid (0.175 g, 31% yield): MS (ES+) m/z 554.6 (M+1).

### Step 2. Preparation of N-(4-(2,5-difluorophenyl)-2-((6R)-6-methylmorpholin-2-yl)pyridin-3-yl)-2-isopropylpyrimidine-5-carboxamide hydrochloric formic acid mixed salt

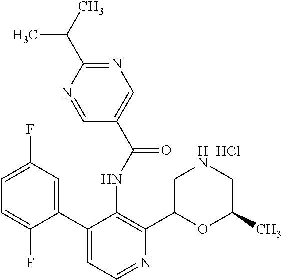 [SMILES: CC(C)c1ncc(C(=O)Nc2c(-c3cc(F)ccc3F)ccnc2C2CNC[C@@H](C)O2)cn1 ; Cl]

To tert-butyl (6R)-2-(4-(2,5-difluorophenyl)-3-(2-isopropylpyrimidine-5-carboxamido)pyridin-2-yl)-6-methylmorpholine-4-carboxylate (0.175 g, 0.315 mmol) was added 4.0 M hydrochloric acid in anhydrous dioxane (2.40 mL, 9.46 mmol). The reaction mixture was stirred at ambient temperature for 16 h. The reaction mixture was concentrated in vacuo. The residue was purified by reverse phase column chromatography, eluting with a gradient of 5 to 95% of acetonitrile in water containing 0.5% formic acid, to afford the title compound as a colorless solid (0.084 g, 54% yield): ¹H NMR (400 MHz, DMSO-d₆, dr=3:2) δ 10.40 (s, 2H), 10.35 (s, 3H), 9.00 (s, 4H), 8.98 (s, 6H), 8.66 (dd, J=4.9, 1.4 Hz, 5H), 8.24 (s, 1.25H), 7.51 (d, J=5.0 Hz, 3H), 7.49 (d, J=4.9 Hz, 2H), 7.36 (td, J=9.0, 4.3 Hz, 5H), 7.32-7.25 (m, 10H), 5.06 (t, J=4.0 Hz, 3H), 4.81 (t, J=6.3 Hz, 2H), 3.98-3.07 (m, 25H), 2.97-2.91 (m, 5H), 2.80 (ddd, J=15.8, 12.9, 2.7 Hz, 5H), 2.44 (dd, J=12.9, 7.1 Hz, 3H), 2.33 (dd, J=12.5, 10.6 Hz, 2H), 1.28 (dd, J=6.9, 1.6 Hz, 30H), 1.00 (d, J=6.2 Hz, 6H), 0.97 (d, J=6.4 Hz, 9H); MS (ES+) m/z 454.3 (M+1).

### Example 615

### Synthesis of 3-(2,2-difluoroethoxy)-N-(4-(2,5-difluorophenyl)-2-(1,1-dioxidotetrahydro-2H-thiopyran-4-yl)pyridin-3-yl)isoxazole-5-carboxamide

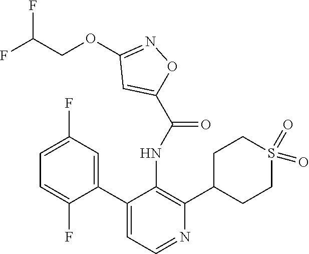 [SMILES: O=C(Nc1c(-c2cc(F)ccc2F)ccnc1C1CCS(=O)(=O)CC1)c1cc(OCC(F)F)no1]

### Step 1. Preparation of 4-(3-amino-4-(2,5-difluorophenyl)pyridin-2-yl)tetrahydro-2H-thiopyran 1,1-dioxide

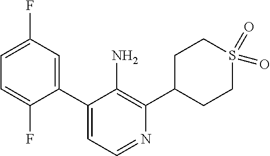 [SMILES: Nc1c(-c2cc(F)ccc2F)ccnc1C1CCS(=O)(=O)CC1]

To a vial containing tert-butyl (2-chloro-4-(2,5-difluorophenyl)pyridin-3-yl)carbamate (0.250 g, 0.734 mmol), cesium carbonate (0.502 g, 1.54 mmol), nickel(II) chloride dimethoxyethane adduct (0.016 g, 0.073 mmol), 4-tert-butyl-2-(4-tert-butyl-2-pyridyl)pyridine (0.030 g, 0.11 mmol), (4,4″-di-tert-butyl-2,2″-bipyridine)bis[3,5-difluoro-2-[5-trifluoromethyl-2-pyridinyl-kN)phenyl-kC]iridium(III) hexafluorophosphate (0.008 g, 0.007 mmol) and tetrahydro-2H-thiopyran-4-carboxylic acid 1,1-dioxide (0.262 g, 1.47 mmol) was added anhydrous N,N-dimethylformamide (18.3 mL) and the headspace was flushed with nitrogen for 10 seconds. The vial was sealed and placed in front of 4 Kessil PR160L lights (440 nm) for 20 h. The reaction mixture was diluted with ethyl acetate (150 mL) and the organic phase was washed with saturated sodium bicarbonate (50 mL), saturated ammonium chloride (50 mL), water (3×50 mL), and brine (50 mL), then dried over anhydrous magnesium sulfate, filtered, and concentrated in vacuo. To the residue was added 4.0 M hydrochloric acid in anhydrous dioxane (4.50 mL, 18.3 mmol) and the reaction mixture was stirred at ambient temperature for 3.5 h. The reaction mixture was concentrated in vacuo. The residue was purified by column chromatography, using a gradient of 10 to 100% of ethyl acetate containing 10% triethylamine and 10% 2-propanol in heptane as eluent, to afford the title compound as a yellow solid (0.151 g, 61% yield): MS (ES+) m/z 339.2 (M+1).

### Step 2. Preparation of 3-(2,2-difluoroethoxy)-N-(4-(2,5-difluorophenyl)-2-(1,1-dioxidotetrahydro-2H-thiopyran-4-yl)pyridin-3-yl)isoxazole-5-carboxamide

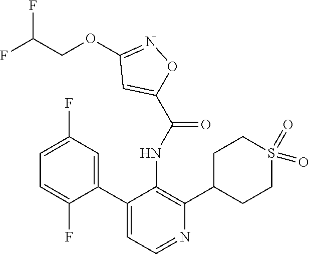 [SMILES: O=C(Nc1c(-c2cc(F)ccc2F)ccnc1C1CCS(=O)(=O)CC1)c1cc(OCC(F)F)no1]

To a mixture of 4-(3-amino-4-(2,5-difluorophenyl)pyridin-2-yl)tetrahydro-2H-thiopyran 1,1-dioxide (0.075 g, 0.22 mmol) in anhydrous tetrahydrofuran (4.4 mL) was added N,N-diisopropylethylamine (0.39 mL, 2.2 mmol), 2-chloro-1-methylpyridinium iodide (0.170 g, 0.665 mmol) and 2-isopropylpyrimidine-5-carboxylic acid (0.064 g, 0.33 mmol). The reaction mixture was stirred at 50° C. for 16 h. After cooling to ambient temperature, the reaction mixture was diluted with methanol (4.5 mL) and 5 M sodium hydroxide (1.3 mL) and stirred and ambient temperature for 5 minutes. The reaction mixture was diluted with ethyl acetate (100 mL), and the organic phase was washed with water (30 mL) and saturated ammonium chloride (30 mL), dried over anhydrous magnesium sulfate, filtered, and concentrated in vacuo. The residue was purified by reverse phase column chromatography, eluting with a gradient of 10 to 100% of acetonitrile in water, to afford the title compound as a colorless solid (0.086 g, 75% yield): ¹H NMR (400 MHz, DMSO-d₆) δ 10.66 (s, 1H), 8.65 (d, J=4.9 Hz, 1H), 7.41-7.39 (m, 1H), 7.38-7.27 (m, 2H), 7.22 (ddd, J=8.7, 5.6, 3.1 Hz, 1H), 6.98 (s, 1H), 6.42 (tt, J=53.8, 3.1 Hz, 1H), 4.57 (td, J=15.0, 3.1 Hz, 2H), 3.42-3.28 (m, 3H), 3.10 (d, J=12.2 Hz, 2H), 2.36-2.26 (m, 2H), 2.04 (d, J=12.5 Hz, 2H); MS (ES+) m/z 514.2 (M+1).

### Example 616

### Synthesis of N-(2-(3,3-difluorocyclopentyl)-4-(2,5-difluorophenyl)pyridin-3-yl)-2-isopropoxypyrimidine-5-carboxamide

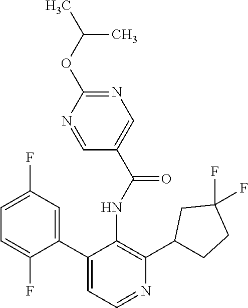 [SMILES: CC(C)Oc1ncc(C(=O)Nc2c(-c3cc(F)ccc3F)ccnc2C2CCC(F)(F)C2)cn1]

### Step 1. Preparation of 2-chloro-N-(2-chloro-4-(2,5-difluorophenyl)pyridin-3-yl)pyrimidine-5-carboxamide

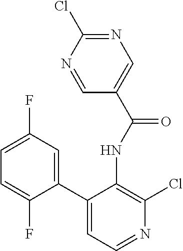 [SMILES: O=C(Nc1c(-c2cc(F)ccc2F)ccnc1Cl)c1cnc(Cl)nc1]

To tert-butyl (2-chloro-4-(2,5-difluorophenyl)pyridin-3-yl)carbamate (2.00 g, 5.87 mmol) was added 4.0 M hydrochloric acid in anhydrous dioxane (20.0 mL, 192 mmol) and the reaction mixture was stirred at 45° C. temperature for 2 h. To the crude material was added anhydrous tetrahydrofuran (120 mL), N,N-diisopropylethylamine (15.5 mL, 88.0 mmol), 2-chloro-1-methylpyridinium iodide (4.50 g, 17.6 mmol) and 2-chloropyrimidine-5-carboxylic acid (2.79 g, 17.6 mmol). The reaction mixture was stirred at 70° C. for 3.5 h. After cooling to ambient temperature, to the reaction mixture was added 2-chloro-1-methylpyridinium iodide (1.50 g, 5.87 mmol) and 2-chloropyrimidine-5-carboxylic acid (0.465 g, 2.93 mmol). The reaction mixture was stirred at 70° C. for 16 h. After cooling to ambient temperature, the reaction mixture was diluted with saturated ammonium chloride (100 mL), and the aqueous phase was extracted with ethyl acetate (3×100 mL), dried over anhydrous magnesium sulfate, filtered, and concentrated in vacuo. The residue was purified by column chromatography, eluting with a gradient of 5 to 75% of ethyl acetate in heptane, to afford the title compound as an orange solid (0.995 g, 45% yield): MS (ES+) m/z 381.2 (M+1), 383.2 (M+1).

### Step 2. Preparation of N-(2-chloro-4-(2,5-difluorophenyl)pyridin-3-yl)-2-isopropoxypyrimidine-5-carboxamide

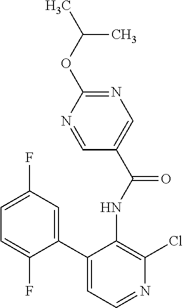 [SMILES: CC(C)Oc1ncc(C(=O)Nc2c(-c3cc(F)ccc3F)ccnc2Cl)cn1]

To a mixture of 2-chloro-N-(2-chloro-4-(2,5-difluorophenyl)pyridin-3-yl)pyrimidine-5-carboxamide (0.995 g, 2.61 mmol) in anhydrous N,N-dimethylformamide (13.0 mL) and 2-propanol (26 mL) at 0° C. was added 60% sodium hydride dispersion in mineral oil (0.157 g, 3.92 mmol). The reaction mixture was stirred at 45° C. for 16 h. After cooling to ambient temperature, to the reaction mixture was added 60% sodium hydride dispersion in mineral oil (0.157 g, 3.92 mmol). The reaction mixture was stirred at 50° C. for 24 h. After cooling to ambient temperature, the reaction mixture was diluted with ethyl acetate (200 mL), and the organic phase was washed with 1 M sodium hydroxide (2×50 mL), water (3×50 mL), and ammonium chloride (2×50 mL). The mixture was dried over anhydrous magnesium sulfate, filtered, and concentrated in vacuo. The residue was purified by column chromatography, eluting with a gradient of 5 to 75% of ethyl acetate in heptane, to afford the title compound as a colorless solid (0.395 g, 37% yield): MS (ES+) m/z 405.2 (M+1), 407.2 (M+1).

### Step 3. Preparation of N-(2-(3,3-difluorocyclopentyl)-4-(2,5-difluorophenyl)pyridin-3-yl)-2-isopropoxypyrimidine-5-carboxamide

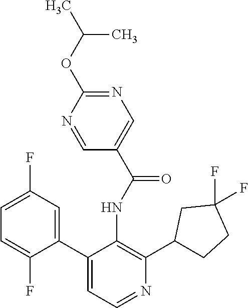 [SMILES: CC(C)Oc1ncc(C(=O)Nc2c(-c3cc(F)ccc3F)ccnc2C2CCC(F)(F)C2)cn1]

To a vial containing N-(2-chloro-4-(2,5-difluorophenyl)pyridin-3-yl)-2-isopropoxypyrimidine-5-carboxamide (0.250 g, 0.618 mmol), cesium carbonate (0.282 g, 0.865 mmol), nickel(II) chloride dimethoxyethane adduct (0.014 g, 0.062 mmol), 4-tert-butyl-2-(4-tert-butyl-2-pyridyl)pyridine (0.025 g, 0.093 mmol), (4,4″-di-tert-butyl-2,2″-bipyridine)bis[3,5-difluoro-2-[5-trifluoromethyl-2-pyridinyl-kN)phenyl-kC]iridium(III) hexafluorophosphate (0.007 g, 0.006 mmol) and 3,3-difluorocyclopentanecarboxylic acid (0.121 g, 0.803 mmol) was added anhydrous N,N-dimethylformamide (15.4 mL) and the headspace was flushed with nitrogen for 10 seconds. The vial was sealed and placed in front of 4 Kessil PR160L lights (440 nm) for 16 h. The reaction mixture was diluted with ethyl acetate (100 mL) and the organic phase was washed with saturated sodium bicarbonate (2×50 mL) and saturated ammonium chloride (2×50 mL), dried over anhydrous magnesium sulfate, filtered, and concentrated in vacuo. The residue was purified by column chromatography, using a gradient of 15 to 75% of ethyl acetate in heptane as eluent, to afford the title compound as a colorless solid (0.088 g, 30% yield): ¹H NMR (400 MHz, DMSO-d₆) δ 10.21 (s, 1H), 8.87 (s, 2H), 8.65 (d, J=4.9 Hz, 1H), 7.41 (d, J=4.9 Hz, 1H), 7.34 (td, J=9.1, 4.6 Hz, 1H), 7.30-7.21 (m, 2H), 5.26 (sept, J=6.2 Hz, 1H), 3.86-3.77 (m, 1H), 2.67-2.38 (m, 2H), 2.32-1.92 (m, 4H), 1.33 (d, J=6.2 Hz, 6H); MS (ES+) m/z 475.2 (M+1).

### Example 617

### Synthesis of N-(2-(syn-4,4-difluoro-2-methylcyclohexyl)-4-(2,5-difluorophenyl)pyridin-3-yl)-2-isopropylpyrimidine-5-carboxamide

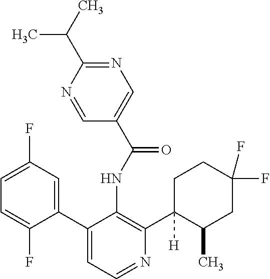 [SMILES: CC(C)c1ncc(C(=O)Nc2c(-c3cc(F)ccc3F)ccnc2[C@H]2CCC(F)(F)C[C@H]2C)cn1]

### Step 1. Preparation of tert-butyl (2-(4,4-difluoro-6-methylcyclohex-1-en-1-yl)-4-(2,5-difluorophenyl)pyridin-3-yl)carbamate and tert-butyl (2-(4,4-difluoro-2-methylcyclohex-1-en-1-yl)-4-(2,5-difluorophenyl)pyridin-3-yl)carbamate

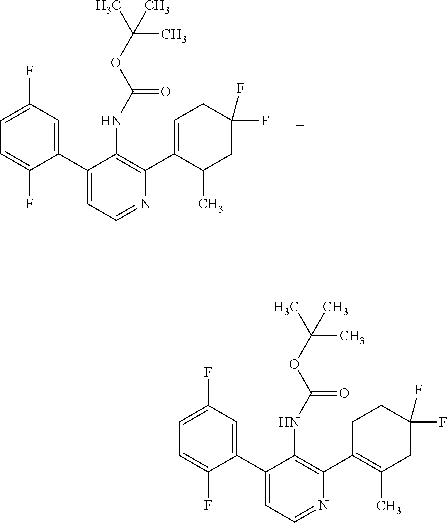 [SMILES: CC1CC(F)(F)CC=C1c1nccc(-c2cc(F)ccc2F)c1NC(=O)OC(C)(C)C ; CC1=C(c2nccc(-c3cc(F)ccc3F)c2NC(=O)OC(C)(C)C)CCC(F)(F)C1]

A mixture of tert-butyl (2-chloro-4-(2,5-difluorophenyl)pyridin-3-yl)carbamate (3.30 g, 9.70 mmol) in dioxane (59 mL) was degassed with nitrogen for 10 minutes. To the reaction mixture was added N—[(Z)-(4,4-difluoro-2-methyl-cyclohexylidene)amino]-4-methyl-benzenesulfonamide (4.60 g, 14.5 mmol),¹ tris(dibenzylideneacetone)dipalladium(0) (0.886 g, 0.968 mmol), tricyclohexylphosphine tetrafluoroborate (0.749 g, 2.03 mmol) and lithium tert-butoxide (2.33 g, 29.1 mmol). The reaction mixture was stirred at 100° C. for 16 h. After cooling to ambient temperature, the reaction mixture was diluted with water (300 mL) and the aqueous phase was extracted with ethyl acetate (3×200 mL). The combined organic phases were washed with brine (200 mL), dried over anhydrous magnesium sulfate, filtered and concentrated in vacuo. The residue was purified by column chromatography, eluting with a gradient of 5 to 40% of ethyl acetate in heptane, to afford the title compounds as an orange solid (3.35 g, 79% yield): MS (ES+) m/z 437.4 (M+1). ¹The methyl hydrazone was synthesized following a literature procedure: J. Org. Chem. 2021, 86, 7333.

### Step 2. 2-(4,4-difluoro-6-methylcyclohex-1-en-1-yl)-4-(2,5-difluorophenyl)pyridin-3-amine and 2-(4,4-difluoro-2-methylcyclohex-1-en-1-yl)-4-(2,5-difluorophenyl)pyridin-3-amine

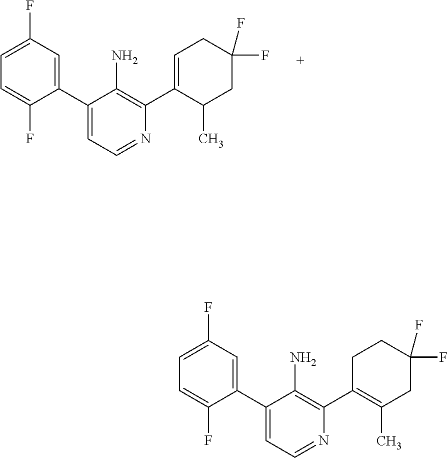 [SMILES: CC1CC(F)(F)CC=C1c1nccc(-c2cc(F)ccc2F)c1N ; CC1=C(c2nccc(-c3cc(F)ccc3F)c2N)CCC(F)(F)C1]

To a mixture of tert-butyl (2-(4,4-difluoro-6-methylcyclohex-1-en-1-yl)-4-(2,5-difluorophenyl)pyridin-3-yl)carbamate and tert-butyl (2-(4,4-difluoro-2-methylcyclohex-1-en-1-yl)-4-(2,5-difluorophenyl)pyridin-3-yl)carbamate (3.35 g, 7.68 mmol) was added 4.0 M hydrochloric acid in anhydrous dioxane (48.0 mL, 192 mmol) and the reaction mixture was stirred at ambient temperature for 16 h. The reaction mixture was diluted with saturated potassium carbonate (100 mL) and the aqueous phase was extracted with ethyl acetate (3×200 mL). The combined organic phase was washed with brine (200 mL), dried over anhydrous magnesium sulfate, filtered and concentrated in vacuo. The residue was purified by column chromatography, eluting with a gradient of 15 to 65% of ethyl acetate in heptane. Further purification by reverse-phase column chromatography, using a gradient of 5 to 35% of acetonitrile in water containing 0.5% formic acid as eluent, afforded the title compounds as colorless solid (1.67 g, 65% yield): MS (ES+) m/z 337.2 (M+1).

### Step 3. Preparation of (2-(syn-4,4-difluoro-2-methylcyclohexyl)-4-(2,5-difluorophenyl)pyridin-3-amine

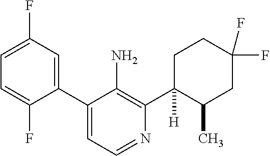 [SMILES: C[C@@H]1CC(F)(F)CC[C@@H]1c1nccc(-c2cc(F)ccc2F)c1N]

A mixture of 2-(4,4-difluoro-6-methylcyclohex-1-en-1-yl)-4-(2,5-difluorophenyl)pyridin-3-amine and 2-(4,4-difluoro-2-methylcyclohex-1-en-1-yl)-4-(2,5-difluorophenyl)pyridin-3-amine (1.57 g, 4.66 mmol) in ethanol (78 mL) and acetic acid (47 mL) was degassed with nitrogen for 10 minutes. To the reaction mixture was added ammonium formate (8.81 g, 139 mmol) and 20% palladium hydroxide on carbon (1.31 g). The reaction mixture was stirred at 80° C. for 2.5 h. After cooling to ambient temperature, the mixture was diluted with ethyl acetate (500 mL) and filtered through a bed of Celite. The filtrate was washed with sodium bicarbonate (150 mL), brine (150 mL), dried over anhydrous magnesium sulfate, and concentrated in vacuo. The residue was purified by column chromatography, eluting with a gradient of 5 to 35% of ethyl acetate in heptane. The residue was further purified by column chromatography, eluting with a gradient of 10 to 25% of ethyl acetate in heptane, to afford the title compound as a colorless solid (0.800 g, 51% yield): ¹H NMR (400 MHz, CDCl₃) δ 8.05 (d, J=4.9 Hz, 1H), 7.18 (tdd, J=8.8, 4.5, 0.5 Hz, 1H), 7.13-7.06 (m, 2H), 6.89 (d, J=4.8 Hz, 1H), 3.66 (s, 2H), 3.02 (dd, J=5.1, 4.2 Hz, 1H), 2.67-2.53 (m, 1H), 2.46-2.21 (m, 3H), 2.05-1.82 (m, 3H), 0.92-0.90 (m, 3H); MS (ES+) m/z 399.2 (M+1).

### Step 4. Preparation of N-(2-syn-4,4-difluoro-2-methylcyclohexyl)-4-(2,5-difluorophenyl)pyridin-3-yl)-2-isopropylpyrimidine-5-carboxamide

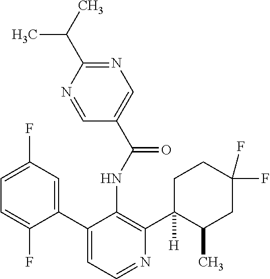 [SMILES: CC(C)c1ncc(C(=O)Nc2c(-c3cc(F)ccc3F)ccnc2[C@H]2CCC(F)(F)C[C@H]2C)cn1]

To a mixture of (2-(syn-4,4-difluoro-2-methylcyclohexyl)-4-(2,5-difluorophenyl)pyridin-3-amine (0.088 g, 0.26 mmol) in anhydrous tetrahydrofuran (5.20 mL) was added N,N-diisopropylethylamine (0.46 mL, 2.6 mmol), 2-chloro-1-methylpyridinium iodide (0.200 g, 0.784 mmol) and 2-isopropylpyrimidine-5-carboxylic acid (0.065 g, 0.39 mmol). The reaction mixture was stirred at 65° C. for 8 h. After cooling to ambient temperature, the reaction mixture was diluted with methanol (5 mL) and 10 M sodium hydroxide (1 mL) and stirred at ambient temperature for 10 minutes. The reaction mixture was diluted with ethyl acetate (100 mL), and the organic phase was washed with 1 M sodium hydroxide (30 mL) and saturated ammonium chloride (30 mL), dried over anhydrous magnesium sulfate, filtered, and concentrated in vacuo. The residue was purified by column chromatography, eluting with a gradient of 10 to 50% of ethyl acetate in heptane, to afford the title compound as a colorless solid (0.98 g, 77% yield): ¹H NMR (400 MHz, DMSO-d₆) δ 10.36 (s, 1H), 8.94 (s, 2H), 8.63 (d, J=4.9 Hz, 1H), 7.40-7.33 (m, 2H), 7.29-7.24 (m, 2H), 3.50-3.43 (m, 1H), 3.19 (sept, J=6.9 Hz, 1H), 2.44-2.39 (m, 1H), 2.33-2.18 (m, 2H), 2.11-1.79 (m, 4H), 1.28 (d, J=6.9 Hz, 6H), 0.74 (d, J=5.4 Hz, 3H); MS (ES+) m/z 487.2 (M+1).

### Example 618-621

In a similar manner as described in Example 617, utilizing the appropriately substituted starting materials and intermediates, the following compounds were prepared:

| Example | Structure | Amount | MS (ES+) | |
|---|---|---|---|---|
| No. | Name | Yield | m/z | ¹H NMR |
| 618 | 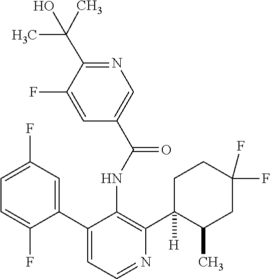 [SMILES: C[C@@H]1CC(F)(F)CC[C@@H]1c1nccc(-c2cc(F)ccc2F)c1NC(=O)c1cnc(C(C)(C)O)c(F)c1] | 0.147 g 85% | 520.4 (M + 1) | (400 MHz, DMSO-d₆) δ 10.32 (s, 1H), 8.63 (d, J = 4.9 Hz, 1H), 8.62 (t, J = 1.6 Hz, 1H), 7.88 (dd, J = 11.9, 1.5 Hz, 1H), 7.39 (d, J = 4.9 Hz, 1H), 7.34 (dt, J = 9.3, 4.6 Hz, 1H), 7.26-7.24 (m, 2H), 5.34 (s, 1H), 3.47-3.43 (m, 1H), 2.57-2.41 (m, 1H), 2.33- 2.03 (m, 3H), 1.99-1.79 (m, 3H), 1.51 (d, J = 0.5 Hz, 6H), 0.74 (s, 3H) |
| | N-(2-(syn-4,4-difluoro-2-methylcyclohexyl)- | | | |
| | 4-(2,5-difluorophenyl)pyridin-3-yl)-5- | | | |
| | fluoro-6-(2-hydroxypropan-2-yl)nicotinamide | | | |
| 619 | 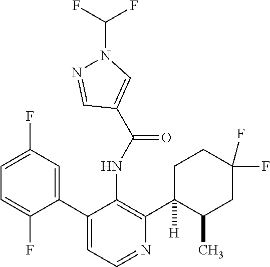 [SMILES: C[C@@H]1CC(F)(F)CC[C@@H]1c1nccc(-c2cc(F)ccc2F)c1NC(=O)c1cnn(C(F)F)c1] | 0.108 g 72% | 483.2 (M + 1) | (400 MHz, CD₃CN) δ 8.59 (d, J = 4.9 Hz, 1H), 8.31 (d, J = 0.5 Hz, 2H), 7.95 (s, 1H), 7.37 (t, J = 59.5 Hz, 1H), 7.30 (dd, J = 4.9, 0.8 Hz, 1H), 7.19-7.07 (m, 3H), 2.65- 2.47 (m, 1H), 2.63-2.49 (m, 1H), 2.42-2.23 (m, 2H), 2.09- 2.06 (m, 1H), 1.91-1.84 (m, 3H), 0.78 (d, J = 7.1 Hz, 3H) |
| | N-(2-(syn-4,4-difluoro-2-methylcyclohexyl)- | | | |
| | 4-(2,5-difluorophenyl)pyridin-3-yl)-1- | | | |
| | (difluoromethyl)-1H-pyrazole-4-carboxamide | | | |
| 620 | 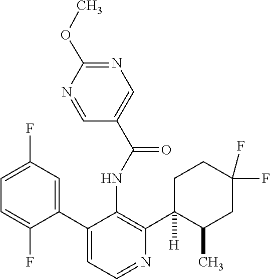 [SMILES: COc1ncc(C(=O)Nc2c(-c3cc(F)ccc3F)ccnc2[C@H]2CCC(F)(F)C[C@H]2C)cn1] | 0.114 g 54% | 475.2 (M + 1) | (400 MHz, DMSO-d₆) δ 10.22 (s, 1H), 8.87 (s, 2H), 8.63 (d, J = 4.9 Hz, 1H), 7.39 (dd, J = 4.9, 0.7 Hz, 1H), 7.34 (td, J = 9.3, 4.4 Hz, 1H), 7.25 (td, J = 7.5, 4.4 Hz, 2H), 3.97 (s, 3H), 3.46 (s, 1H), 2.50-2.39 (m, 1H), 2.34-2.18 (m, 2H), 2.13-2.04 (m, 1H), 2.01-1.79 (m, 3H), 0.74 (d, J = 5.1 Hz, 3H) |
| | N-(2-(syn-4,4-difluoro-2-methylcyclohexyl)- | | | |
| | 4-(2,5-difluorophenyl)pyridin-3-yl)-2- | | | |
| | methoxypyrimidine-5-carboxamide | | | |
| 621 | 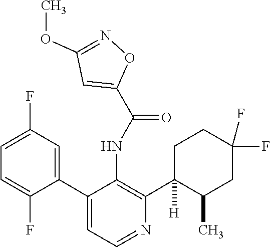 [SMILES: COc1cc(C(=O)Nc2c(-c3cc(F)ccc3F)ccnc2[C@H]2CCC(F)(F)C[C@H]2C)on1] | 0.171 g 83% | 464.2 (M + 1) | (400 MHz, DMSO-d₆) δ 10.64 (s, 1H), 8.63 (d, J = 4.9 Hz, 1H), 7.39-7.22 (m, 4H), 6.83 (s, 1H), 3.93 (s, 3H), 3.39 (s, 1H), 2.59-2.44 (m, 1H), 2.30-2.17 (m, 2H), 2.06-1.79 (m, 4H), 0.73 (d, J = 6.8 Hz, 3H) |
| | N-(2-syn-4,4-difluoro-2-methylcyclohexyl)- | | | |
| | 4-(2,5-difluorophenyl)pyridin-3- | | | |
| | yl)-3-methoxyisoxazole-5-carboxamide | | | |

### Example 622

### Synthesis of N-(2-(anti-4,4-difluoro-2-methylcyclohexyl)-4-(2,5-difluorophenyl)pyridin-3-yl)-2-isopropylpyrimidine-5-carboxamide

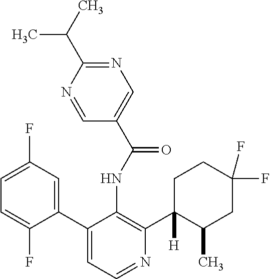 [SMILES: CC(C)c1ncc(C(=O)Nc2c(-c3cc(F)ccc3F)ccnc2[C@@H]2CCC(F)(F)C[C@H]2C)cn1]

### Step 1. Preparation of 2-(anti-4,4-difluoro-2-methylcyclohexyl)-4-(2,5-difluorophenyl)pyridin-3-amine

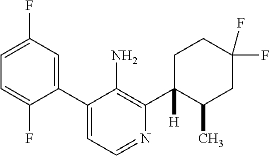 [SMILES: C[C@@H]1CC(F)(F)CC[C@H]1c1nccc(-c2cc(F)ccc2F)c1N]

A mixture of 2-(4,4-difluoro-6-methylcyclohex-1-en-1-yl)-4-(2,5-difluorophenyl)pyridin-3-amine and 2-(4,4-difluoro-2-methylcyclohex-1-en-1-yl)-4-(2,5-difluorophenyl)pyridin-3-amine (1.57 g, 4.66 mmol) in ethanol (78 mL) and acetic acid (47 mL) was degassed with nitrogen for 10 minutes. To the reaction mixture was added ammonium formate (8.81 g, 139 mmol) and 20% palladium hydroxide on carbon (1.31 g). The reaction mixture was stirred at 80° C. for 2.5 h. After cooling to ambient temperature, the mixture was diluted with ethyl acetate (500 mL) and filtered through a bed of Celite. The filtrate was washed with sodium bicarbonate (150 mL), brine (150 mL), dried over anhydrous magnesium sulfate, and concentrated in vacuo. The residue was purified by column chromatography, eluting with a gradient of 5 to 35% of ethyl acetate in heptane. The residue was further purified by column chromatography, eluting with a gradient of 10 to 25% of ethyl acetate in heptane to afford the title compound as a colorless solid (0.369 g, 23% yield): ¹H NMR (400 MHz, CDCl₃) δ 8.11 (d, J=4.8 Hz, 1H), 7.18 (td, J=8.8, 4.5 Hz, 1H), 7.14-7.06 (m, 2H), 6.89 (d, J=4.8 Hz, 1H), 3.66 (s, 2H), 2.51-2.40 (m, 2H), 2.31-2.20 (m, 2H), 2.08-1.98 (m, 1H), 1.92-1.74 (m, 2H), 1.61 (dtd, J=34.8, 13.0, 3.1 Hz, 1H), 0.83 (d, J=6.1 Hz, 3H); MS (ES+) m/z 399.2 (M+1).

### Step 2. Preparation of N-(2-(anti-4,4-difluoro-2-methylcyclohexyl)-4-(2,5-difluorophenyl)pyridin-3-yl)-2-isopropylpyrimidine-5-carboxamide

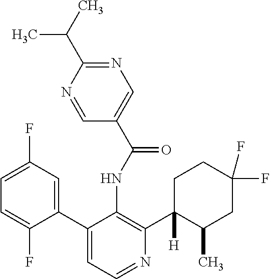 [SMILES: CC(C)c1ncc(C(=O)Nc2c(-c3cc(F)ccc3F)ccnc2[C@@H]2CCC(F)(F)C[C@H]2C)cn1]

To a mixture of 2-(anti-4,4-difluoro-2-methylcyclohexyl)-4-(2,5-difluorophenyl)pyridin-3-amine (0.022 g, 0.064 mmol) in anhydrous tetrahydrofuran (1.30 mL) was added N,N-diisopropylethylamine (0.11 mL, 0.64 mmol), 2-chloro-1-methylpyridinium iodide (0.049 g, 0.19 mmol) and 2-isopropylpyrimidine-5-carboxylic acid (0.016 g, 0.10 mmol). The reaction mixture was stirred at 65° C. for 8 h. After cooling to ambient temperature, to the reaction mixture was added in methanol (3 mL) and 10 M sodium hydroxide (1 mL) and stirred at ambient temperature for 1 h. The reaction mixture was diluted with ethyl acetate (75 mL), and the organic phase was washed with 1 M sodium hydroxide (25 mL), saturated ammonium chloride (25 mL), dried over anhydrous magnesium sulfate, filtered, and concentrated in vacuo. The residue was purified by column chromatography, eluting with a gradient of 10 to 45% of ethyl acetate in heptane, to afford the title compound as a colorless solid (0.013 g, 41% yield): ¹H NMR (300 MHz, DMSO-d₆) 10.36 (s, 1H), 8.95 (s, 2H), 8.65 (d, J=4.9 Hz, 1H), 7.39-7.23 (m, 4H), 3.20 (sept, J=6.9 Hz, 1H), 2.85 (t, J=9.0 Hz, 1H), 2.31-2.20 (m, 1H), 2.12-2.03 (m, 2H), 1.96-1.63 (m, 4H), 1.28 (d, J=6.9 Hz, 6H), 0.64 (d, J=4.3 Hz, 3H); MS (ES+) m/z 487.2 (M+1).

### Example 623-624

In a similar manner as described in Example 622, utilizing the appropriately substituted starting materials and intermediates, the following compounds were prepared:

| Example | Structure | Amount | MS (ES+) | |
|---|---|---|---|---|
| No. | Name | Yield | m/z | ¹H NMR |
| 623 | 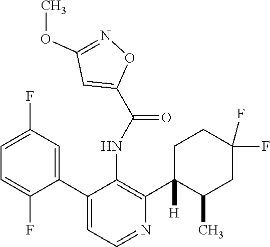 [SMILES: COc1cc(C(=O)Nc2c(-c3cc(F)ccc3F)ccnc2[C@@H]2CCC(F)(F)C[C@H]2C)on1] | 0.143 g 84% | 464.2 (M + 1) | (400 MHz, DMSO-d₆) δ 10.64 (d, J = 0.3 Hz, 1H), 8.64 (d, J = 4.9 Hz, 1H), 7.37-7.22 (m, 4H), 6.84 (s, 1H), 3.94 (s, 3H), 2.77 (t, J = 9.9 Hz, 1H), 2.29-2.22 (m, 1H), 2.11-2.05 (m, 2H), 1.91- 1.84 (m, 1H), 1.80-1.70 (m, 2H), 1.70-1.59 (m, 1H), 0.61 (d, J= 6.1 Hz, 3H) |
| | N-(2-(anti-4,4-difluoro-2-methylcyclohexyl)- | | | |
| | 4-(2,5-difluorophenyl)pyridin-3-yl)-3- | | | |
| | methoxyisoxazole-5-carboxamide | | | |
| 624 | 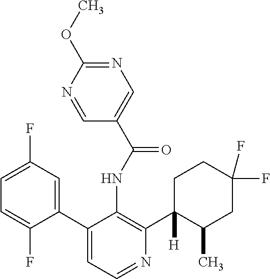 [SMILES: COc1ncc(C(=O)Nc2c(-c3cc(F)ccc3F)ccnc2[C@@H]2CCC(F)(F)C[C@H]2C)cn1] | 0.122 g 70% | 475.4 (M + 1) | (400 MHz, DMSO-d₆) δ 10.22 (s, 1H), 8.88 (s, 2H), 8.64 (d, J = 4.9 Hz, 1H), 7.34 (dq, J = 9.5, 4.6 Hz, 2H), 7.28-7.22 (m, 2H), 3.98 (s, 3H), 2.84 (t, J = 9.2 Hz, 1H), 2.30-2.22 (m, 1H), 2.08-2.06 (m, 2H), 1.91-1.61 (m, 4H), 0.64 (d, J = 3.3 Hz, 3H) |
| | N-(2-(anti-4,4-difluoro-2-methylcyclohexyl)- | | | |
| | 4-(2,5-difluorophenyl)pyridin-3- | | | |
| | yl)-2-methoxypyrimidine-5-carboxamide | | | |

### Example 625

### Synthesis of N-(2-(4,4-difluoro-2-methylcyclohex-1-en-1-yl)-4-(2,5-difluorophenyl)pyridin-3-yl)-2-isopropylpyrimidine-5-carboxamide

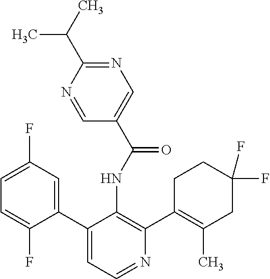 [SMILES: CC1=C(c2nccc(-c3cc(F)ccc3F)c2NC(=O)c2cnc(C(C)C)nc2)CCC(F)(F)C1]

### Step 1. Preparation of 2-(4,4-difluoro-2-methylcyclohex-1-en-1-yl)-4-(2,5-difluorophenyl)pyridin-3-amine

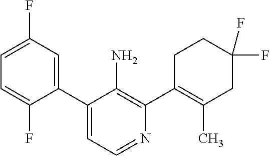 [SMILES: CC1=C(c2nccc(-c3cc(F)ccc3F)c2N)CCC(F)(F)C1]

A mixture of 2-(4,4-difluoro-6-methylcyclohex-1-en-1-yl)-4-(2,5-difluorophenyl)pyridin-3-amine and 2-(4,4-difluoro-2-methylcyclohex-1-en-1-yl)-4-(2,5-difluorophenyl)pyridin-3-amine (1.57 g, 4.66 mmol) in ethanol (78 mL) and acetic acid (47 mL) was degassed with nitrogen for 10 minutes. To the reaction mixture was added ammonium formate (8.81 g, 139 mmol) and 20% palladium hydroxide on carbon (1.31 g). The reaction mixture was stirred at 80° C. for 2.5 h. After cooling to ambient temperature, the mixture was diluted with ethyl acetate (500 mL) and filtered through a bed of Celite. The filtrate was washed with sodium bicarbonate (150 mL), brine (150 mL), dried over anhydrous magnesium sulfate, and concentrated in vacuo. The residue was purified by column chromatography, eluting with a gradient of 5 to 35% of ethyl acetate in heptane. The residue was further purified by column chromatography, eluting with a gradient of 10 to 25% of ethyl acetate in heptane to afford the title compound as a colorless solid (0.288 g, 18% yield): ¹H NMR (400 MHz, CDCl₃) δ 8.09 (d, J=4.9 Hz, 1H), 7.21-7.14 (m, 1H), 7.13-7.08 (m, 2H), 6.97 (d, J=4.7 Hz, 1H), 3.75 (s, 2H), 2.74-2.47 (m, 4H), 2.25-2.16 (m, 2H), 1.59 (s, 3H); MS (ES+) m/z 337.4 (M+1).

### Step 2. Preparation of N-(2-(4,4-difluoro-2-methylcyclohex-1-en-1-yl)-4-(2,5-difluorophenyl)pyridin-3-yl)-2-isopropylpyrimidine-5-carboxamide

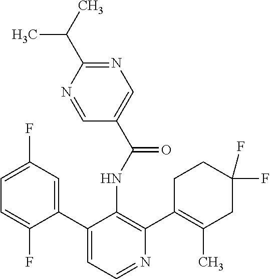 [SMILES: CC1=C(c2nccc(-c3cc(F)ccc3F)c2NC(=O)c2cnc(C(C)C)nc2)CCC(F)(F)C1]

To a mixture 2-(4,4-difluoro-2-methylcyclohex-1-en-1-yl)-4-(2,5-difluorophenyl)pyridin-3-amine (0.120 g, 0.357 mmol) in anhydrous tetrahydrofuran (7.1 mL) was added N,N-diisopropylethylamine (0.93 mL, 5.4 mmol), 2-chloro-1-methylpyridinium iodide (0.322 g, 1.26 mmol) and 2-isopropylpyrimidine-5-carboxylic acid (0.180 g, 3.04 mmol). The reaction mixture was stirred at 65° C. for 16 h. After cooling to ambient temperature, to the reaction mixture was added in methanol (7 mL) and 5 M sodium hydroxide (2 mL). The reaction mixture was stirred at 40 for 15 minutes. After cooling to ambient temperature, the reaction mixture was diluted with ethyl acetate (100 mL), and the organic phase was washed with water (30 mL), saturated ammonium chloride (30 mL), brine (30 mL), dried over anhydrous magnesium sulfate, filtered, and concentrated in vacuo. The residue was purified by column chromatography, eluting with a gradient of 10 to 65% of ethyl acetate in heptane, to afford the title compound as a colorless solid (0.147 g, 85% yield): ¹H NMR (300 MHz, DMSO-d₆) 10.18 (s, 1H), 8.85 (s, 2H), 8.64 (d, J=5.0 Hz, 1H), 7.46-7.44 (m, 1H), 7.38 (td, J=9.2, 4.6 Hz, 1H), 7.34-7.25 (m, 2H), 3.18 (sept, J=6.9 Hz, 1H), 2.60-2.52 (m, 4H), 2.07 (tt, J=13.4, 6.5 Hz, 2H), 1.41 (s, 3H), 1.28 (d, J=6.9 Hz, 6H); MS (ES+) m/z 485.4 (M+1).

### Example 626

### Synthesis of N-(2-(4,4-difluoro-1-hydroxycyclohexyl)-4-(2,5-difluorophenyl)pyridin-3-yl)-2-isopropylpyrimidine-5-carboxamide

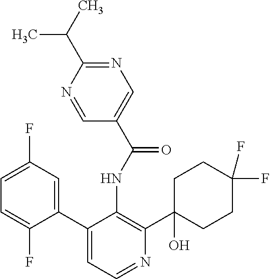 [SMILES: CC(C)c1ncc(C(=O)Nc2c(-c3cc(F)ccc3F)ccnc2C2(O)CCC(F)(F)CC2)cn1]

### Step 1. Preparation of tert-butyl (2-(4,4-difluorocyclohex-1-en-1-yl)-4-(2,5-difluorophenyl)pyridin-3-yl)carbamate

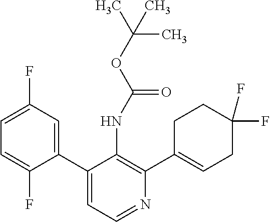 [SMILES: CC(C)(C)OC(=O)Nc1c(-c2cc(F)ccc2F)ccnc1C1=CCC(F)(F)CC1]

A mixture of tert-butyl (2-chloro-4-(2,5-difluorophenyl)pyridin-3-yl)carbamate (5.00 g, 14.7 mmol) in 1,4-dioxane (74 mL) and water (8.0 mL) was degassed with nitrogen for 10 minutes. To the reaction mixture was added 2-(4,4-difluorocyclohex-1-en-1-yl)-4,4,5,5-tetramethyl-1,3,2-dioxaborolane (4.66 g, 19.1 mmol), potassium carbonate (5.07 g, 36.7 mmol), and dichloro[1,1′-bis(diphenyl-phosphino)ferrocene]palladium(II) dichloromethane adduct (1.24 g, 1.47 mmol). The reaction mixture was stirred at 80° C. for 4 h. After cooling to ambient temperature, the mixture was diluted with ethyl acetate (300 mL) and filtered through a bed of Celite. The filtrate was washed with saturated ammonium chloride (100 mL), dried over anhydrous magnesium sulfate, and concentrated in vacuo. The residue was purified by column chromatography, eluting with a gradient of 5 to 35% of ethyl acetate in heptane to afford the title compound as a colorless solid (5.08 g, 82% yield): MS (ES+) m/z 423.4 (M+1).

### Step 2. Preparation of 2-(4,4-difluorocyclohex-1-en-1-yl)-4-(2,5-difluorophenyl)pyridin-3-amine

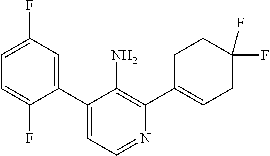 [SMILES: Nc1c(-c2cc(F)ccc2F)ccnc1C1=CCC(F)(F)CC1]

To tert-butyl (2-(4,4-difluoro-6-methylcyclohex-1-en-1-yl)-4-(2,5-difluorophenyl)pyridin-3-yl)carbamate (2.96 g, 7.01 mmol) was added 4.0 M hydrochloric acid in anhydrous dioxane (35.0 mL, 140 mmol) and the reaction mixture was stirred at ambient temperature for 5 h. The reaction mixture was diluted with saturated potassium carbonate (200 mL) and the aqueous phase was extracted with ethyl acetate (3×200 mL). The combined organic phase was washed with brine (200 mL), dried over anhydrous magnesium sulfate, filtered and concentrated in vacuo. Purification of the residue by column chromatography, eluting with a gradient of 10 to 65% of ethyl acetate in heptane afforded the title compound as a yellow oil (2.59 g, 115% yield): MS (ES+) m/z 323.3 (M+1).

### Step 3. Preparation of 1-(3-amino-4-(2,5-difluorophenyl)pyridin-2-yl)-4,4-difluorocyclohexan-1-ol

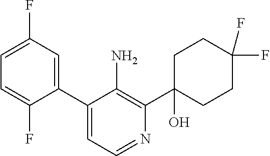 [SMILES: Nc1c(-c2cc(F)ccc2F)ccnc1C1(O)CCC(F)(F)CC1]

To a mixture of 2-(4,4-difluorocyclohex-1-en-1-yl)-4-(2,5-difluorophenyl)pyridin-3-amine (2.59 g, 8.04 mmol) in anhydrous tetrahydrofuran (47.0 mL) at 0° C. was added borane methyl sulfide complex (7.50 mL, 75.0 mmol) and the reaction mixture was left to stir at ambient temperature for 16 h. To the reaction mixture at 0° C. was added 5 M sodium hydroxide (28 mL), 35% hydrogen peroxide in water (9.5 mL, 98 mmol), anhydrous tetrahydrofuran (30 mL) and stirred at ambient temperature for 24 h. The reaction mixture was diluted with saturated ammonium chloride (100 mL), and the aqueous phase was extracted with ethyl acetate (3×200 mL). The combined organic phases were washed with brine (200 mL), dried over anhydrous magnesium sulfate, filtered, and concentrated in vacuo. The residue was purified by reverse phase column chromatography, eluting with a gradient of eluting with a gradient of 5 to 50% of acetonitrile in water with 0.5% formic acid to afford the title compound as a colorless solid (0.837 g, 35% yield): ¹H NMR (400 MHz, CDCl₃) δ 7.98 (d, J=4.7 Hz, 1H), 7.18 (td, J=8.8, 4.5 Hz, 1H), 7.12 (td, J=6.8, 3.1 Hz, 1H), 7.07 (ddd, J=8.4, 5.5, 3.0 Hz, 1H), 6.97 (d, J=4.8 Hz, 1H), 4.10 (s, 2H), 2.44 (t, J=12.2 Hz, 2H), 2.37-2.20 (m, 2H), 2.10 (d, J=9.4 Hz, 4H); MS (ES+) m/z 341.2 (M+1).

### Step 4. Preparation of N-(2-(4,4-difluoro-1-hydroxycyclohexyl)-4-(2,5-difluorophenyl)pyridin-3-yl)-2-isopropylpyrimidine-5-carboxamide

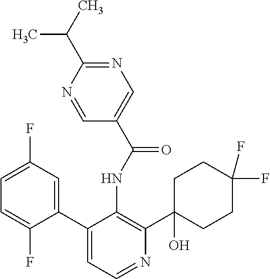 [SMILES: CC(C)c1ncc(C(=O)Nc2c(-c3cc(F)ccc3F)ccnc2C2(O)CCC(F)(F)CC2)cn1]

To a mixture of 1-(3-amino-4-(2,5-difluorophenyl)pyridin-2-yl)-4,4-difluorocyclohexan-1-ol (0.041 g, 0.12 mmol) in anhydrous tetrahydrofuran (2.4 mL) was added N,N-diisopropylethylamine (0.21 mL, 1.2 mmol), 2-chloro-1-methylpyridinium iodide (0.123 g, 0.483 mmol) and 2-isopropylpyrimidine-5-carboxylic acid (0.032 g, 0.19 mmol). The reaction mixture was stirred at 40° C. for 24 h. After cooling to ambient temperature, the reaction mixture was diluted with methanol (3 mL) and 10 M sodium hydroxide (1 mL) and stirred and ambient temperature for 3 h. The reaction mixture was diluted saturated ammonium chloride (50 mL), and extracted with ethyl acetate (3×75 mL). The organic phase was dried over anhydrous magnesium sulfate, filtered, and concentrated in vacuo. The residue was purified by column chromatography, eluting with a gradient of 10 to 65% of ethyl acetate in heptane, to afford the title compound as a colorless solid (0.042 g, 71% yield): ¹H NMR (400 MHz, DMSO-d₆) δ 10.31 (s, 1H), 8.93 (s, 2H), 8.58 (d, J=4.9 Hz, 1H), 7.46 (d, J=4.8 Hz, 1H), 7.38-7.29 (m, 2H), 7.23 (tt, J=8.1, 3.9 Hz, 1H), 5.98 (s, 1H), 3.19 (sept, J=6.9 Hz, 1H), 2.26-2.09 (m, 4H), 1.97 (d, J=9.7 Hz, 4H), 1.28 (d, J=6.9 Hz, 6H); MS (ES+) m/z 489.2 (M+1).

### Example 627

### Synthesis of N-(2-(4,4-difluorocyclohex-1-en-1-yl)-4-(2,5-difluorophenyl)pyridin-3-yl)-2-isopropylpyrimidine-5-carboxamide

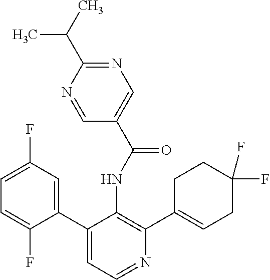 [SMILES: CC(C)c1ncc(C(=O)Nc2c(-c3cc(F)ccc3F)ccnc2C2=CCC(F)(F)CC2)cn1]

### Step 1. Preparation of N-(2-(4,4-difluoro-1-hydroxycyclohexyl)-4-(2,5-difluorophenyl)pyridin-3-yl)-2-isopropylpyrimidine-5-carboxamide

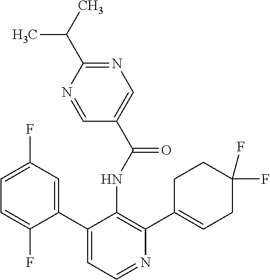 [SMILES: CC(C)c1ncc(C(=O)Nc2c(-c3cc(F)ccc3F)ccnc2C2=CCC(F)(F)CC2)cn1]

To tert-butyl (2-(4,4-difluorocyclohex-1-en-1-yl)-4-(2,5-difluorophenyl)pyridin-3-yl)carbamate (0.150 g, 0.355 mmol) was added 4 M hydrochloric acid in anhydrous 1,4-dioxane (2.70 mL, 10.7 mmol). The reaction mixture was left to stir at ambient temperature for 4 h. The volatiles were removed in vacuo. To the reaction mixture was added anhydrous tetrahydrofuran (7.0 mL), N,N-diisopropylethylamine (1.25 mL, 7.10 mmol), 2-chloro-1-methylpyridinium iodide (0.272 g, 1.07 mmol) and 2-isopropylpyrimidine-5-carboxylic acid (0.089 g, 0.53 mmol). The reaction mixture was stirred at 65° C. for 16 h. After cooling to ambient temperature, to the reaction mixture was added 2-chloro-1-methylpyridinium iodide (0.091 g, 0.36 mmol) and 2-isopropylpyrimidine-5-carboxylic acid (0.030 g, 0.35 mmol). The reaction mixture was stirred at 65° C. for 16 h. After cooling to ambient temperature, to the reaction mixture was added methanol (7.0 mL) and 5 M sodium hydroxide (2.0 mL). The reaction mixture was stirred at ambient temperature for 1 h. The reaction mixture was diluted with ethyl acetate (150 mL) and the organic phase was washed with water (50 mL), saturated ammonium chloride (50 mL), and brine (50 mL). The mixture was dried over anhydrous magnesium sulfate, filtered, and concentrated in vacuo. The residue was purified by column chromatography, eluting with a gradient of 10 to 65% of ethyl acetate in heptane, to afford the title compound as a colorless solid (0.130 g, 78% yield): ¹H NMR (400 MHz, DMSO-d₆) δ 10.32 (s, 1H), 8.88 (s, 2H), 8.63 (d, J=4.9 Hz, 1H), 7.45 (d, J=4.9 Hz, 1H), 7.39 (td, J=9.1, 4.6 Hz, 1H), 7.34-7.25 (m, 2H), 5.79 (s, 1H), 3.19 (sept, J=6.9 Hz, 1H), 2.69 (t, J=6.0 Hz, 2H), 2.61 (t, J=14.5 Hz, 2H), 2.14 (tt, J=13.9, 6.9 Hz, 2H), 1.28 (d, J=6.9 Hz, 6H); MS (ES+) m/z 471.2 (M+1).

### Example 628

### Synthesis of N-(4-(2,5-difluorophenyl)-2-(1,4,4-trifluorocyclohexyl)pyridin-3-yl)-2-isopropylpyrimidine-5-carboxamide

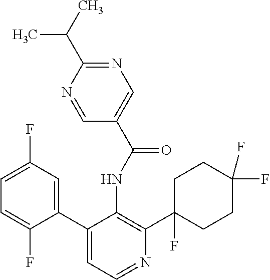 [SMILES: CC(C)c1ncc(C(=O)Nc2c(-c3cc(F)ccc3F)ccnc2C2(F)CCC(F)(F)CC2)cn1]

### Step 1. Preparation of 4-(2,5-difluorophenyl)-2-(1,4,4-trifluorocyclohexyl)pyridin-3-amine

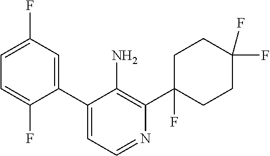 [SMILES: Nc1c(-c2cc(F)ccc2F)ccnc1C1(F)CCC(F)(F)CC1]

To a mixture of 1-(3-amino-4-(2,5-difluorophenyl)pyridin-2-yl)-4,4-difluorocyclohexan-1-ol (0.257 g, 0.756 mmol) in anhydrous dichloromethane (7.6 mL) at 0° C. was added diethylaminosulfur trifluoride (0.50 mL, 3.8 mmol). The reaction mixture was left to stir at 0° C. for 2 h. The reaction mixture was diluted with water (20 mL), and the aqueous phase was extracted with ethyl acetate (3×50 mL). The combined organic phase was washed with brine (200 mL), dried over anhydrous magnesium sulfate, filtered, and concentrated in vacuo. The residue was purified by column chromatography, eluting with a gradient of 5 to 45% of ethyl acetate in heptane to afford the title compound as a colorless solid (0.246 g, 95% yield): ¹H NMR (400 MHz, CDCl₃) 97.99 (dd, J=4.7, 1.0 Hz, 1H), 7.22-7.16 (m, 1H), 7.15-7.11 (m, 1H), 7.10-7.06 (m, 1H), 6.98 (d, J=4.7 Hz, 1H), 4.32 (s, 2H), 2.51-2.36 (m, 4H), 2.30-2.14 (m, 4H); ¹⁹F NMR (376 MHz, CDCl₃) −93.0 (d, J=236 Hz), −103.7 (dd, J=237.7 Hz), −117.5 (d, J=18 Hz), −119.8 (d, J=18 Hz), −165.9; MS (ES+) m/z 343.4 (M+1).

### Step 2. Preparation of N-(4-(2,5-difluorophenyl)-2-(1,4,4-trifluorocyclohexyl)pyridin-3-yl)-2-isopropylpyrimidine-5-carboxamide

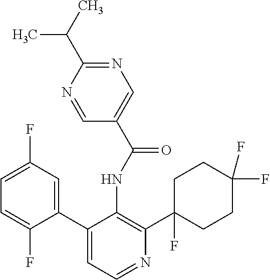 [SMILES: CC(C)c1ncc(C(=O)Nc2c(-c3cc(F)ccc3F)ccnc2C2(F)CCC(F)(F)CC2)cn1]

To a mixture of 4-(2,5-difluorophenyl)-2-(1,4,4-trifluorocyclohexyl)pyridin-3-amine (0.125 g, 0.370 mmol) in anhydrous tetrahydrofuran (7.3 mL) was added N,N-diisopropylethylamine (0.64 mL, 3.7 mmol), 2-chloro-1-methylpyridinium iodide (0.280 g, 1.10 mmol) and 2-isopropylpyrimidine-5-carboxylic acid (0.067 g, 0.40 mmol). The reaction mixture was stirred at 65° C. for 16 h. After cooling to ambient temperature, to the reaction mixture was added 2-chloro-1-methylpyridinium iodide (0.093 g, 0.37 mmol) and 2-isopropylpyrimidine-5-carboxylic acid (0.030 g, 0.183 mmol). The reaction mixture was stirred at 65° C. for 3 h. After cooling to ambient temperature, the reaction mixture was diluted with methanol (7.5 mL) and 10 M sodium hydroxide (1 mL) and stirred and ambient temperature for 10 minutes. The reaction mixture was diluted with saturated ammonium chloride (75 mL) and extracted with ethyl acetate (2×75 mL). The combined organic phase was dried over anhydrous magnesium sulfate, filtered, and concentrated in vacuo. The residue was purified by column chromatography, eluting with a gradient of 10 to 50% of ethyl acetate in heptane, to afford the title compound as a colorless solid (0.132 g, 74% yield): ¹H NMR (400 MHz, DMSO-d₆) δ 10.32 (s, 1H), 8.91 (s, 2H), 8.68 (d, J=4.9 Hz, 1H), 7.57 (dd, J=4.8, 0.7 Hz, 1H), 7.38 (td, J=9.5, 4.5 Hz, 1H), 7.31-7.25 (m, 2H), 3.18 (sept, J=6.9 Hz, 1H), 2.43-2.22 (m, 4H), 2.19-1.99 (m, 4H), 1.27 (d, J=6.9 Hz, 6H); ¹⁹F NMR (376 MHz, DMSO-d₆) δ −91.0 (d, J=234 Hz), −102.5 (d, J=234, 7 Hz), −119.7 (dd, J=503, 14 Hz), −165.8; MS (ES+) m/z 491.2 (M+1).

### Example 629

### Synthesis of N-(2-(syn-4,4-difluoro-2-(methyl-d₃)cyclohexyl)-4-(2,5-difluorophenyl)pyridin-3-yl)-1-(difluoromethyl)-1H-pyrazole-4-carboxamide

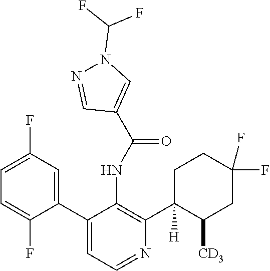 [SMILES: [2H]C([2H])([2H])[C@@H]1CC(F)(F)CC[C@@H]1c1nccc(-c2cc(F)ccc2F)c1NC(=O)c1cnn(C(F)F)c1]

### Step 1. Preparation of N′-(4,4-difluoro-2-(methyl-d₃)cyclohexylidene)-4-methylbenzenesulfonohydrazide and N′-(4,4-difluoro-2,2-bis(methyl-d₃)cyclohexylidene)-4-methylbenzenesulfonohydrazide

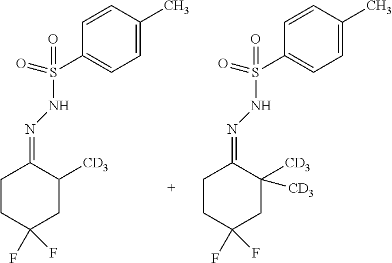 [SMILES: [2H]C([2H])([2H])C1CC(F)(F)CC/C1=N/NS(=O)(=O)c1ccc(C)cc1 ; [2H]C([2H])([2H])C1(C([2H])([2H])[2H])CC(F)(F)CC/C1=N/NS(=O)(=O)c1ccc(C)cc1]

To a mixture of 4,4-difluorocyclohexanone (8.00 g, 60.0 mmol) in anhydrous tetrahydrofuran (240 mL) at −78° C. was added 1 M lithium bis(trimethylsilyl)amide in anhydrous tetrahydrofuran (60.0 mL, 60.0 mmol) dropwise and the reaction mixture was stirred at −78° C. for 20 minutes. To the reaction mixture was added iodomethane-d₃ (3.71 mL, 60.0 mmol) and the reaction mixture was left to warm to ambient temperature for 16 h. The reaction mixture was diluted with saturated ammonium chloride (300 mL) and the aqueous phase was extracted with ethyl acetate (2×300 mL). The combined organic phase was dried over anhydrous magnesium sulfate, filtered, and concentrated in vacuo. To the reaction mixture in methanol (160 mL) was added p-toluenesulfonhydrazide (12.22 g, 65.61 mmol). The reaction mixture was stirred at 70° C. for 16 h. After cooling to ambient temperature, the reaction mixture was concentrated in vacuo. The residue was purified by column chromatography, eluting with a gradient of 10 to 16% of ethyl acetate in heptane, to afford the title compounds as a colorless solid (4.74 g, −30% dimethylated): MS (ES+) m/z 320.4 (M+1), 337.4 (M+1)

### Step 2. Preparation of tert-butyl (2-(4,4-difluoro-6-(methyl-d₃)cyclohex-1-en-1-yl)-4-(2,5-difluorophenyl)pyridin-3-yl)carbamate

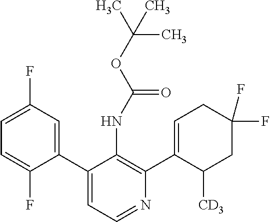 [SMILES: [2H]C([2H])([2H])C1CC(F)(F)CC=C1c1nccc(-c2cc(F)ccc2F)c1NC(=O)OC(C)(C)C]

A mixture of tert-butyl (2-chloro-4-(2,5-difluorophenyl)pyridin-3-yl)carbamate (3.37 g, 9.88 mmol) in dioxane (66 mL) was degassed with nitrogen for 20 minutes. To the reaction mixture was added N′-(4,4-difluoro-2-(methyl-d₃)cyclohexylidene)-4-methylbenzenesulfonohydrazide containing ˜30% N′-(4,4-difluoro-2,2-bis(methyl-d₃)cyclohexylidene)-4-methylbenzenesulfonohydrazide (4.735 g), tris(dibenzylideneacetone)dipalladium(0) (1.36 g, 1.48 mmol), tricyclohexylphosphine tetrafluoroborate (1.13 g, 3.06 mmol) and lithium tert-butoxide (2.77 g, 34.6 mmol). The reaction was stirred at 100° C. for 16 h. After cooling to ambient temperature, the reaction mixture was diluted with ethyl acetate (300 mL) and filtered through a bed of Celite. The filtrate was washed with water (100 mL) and brine (100 mL), dried over anhydrous magnesium sulfate, and concentrated in vacuo. The residue was purified by column chromatography, eluting with a gradient of 10 to 25% of ethyl acetate in heptane to afford the title compound as a red oil (2.37 g, 55% yield): MS (ES+) m/z 440.4 (M+1).

### Step 2. 2-(4,4-difluoro-6-(methyl-d₃)cyclohex-1-en-1-yl)-4-(2,5-difluorophenyl)pyridin-3-amine

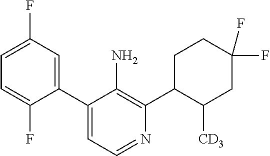 [SMILES: [2H]C([2H])([2H])C1CC(F)(F)CCC1c1nccc(-c2cc(F)ccc2F)c1N]

To a mixture of tert-butyl (2-(4,4-difluoro-6-(methyl-d₃)cyclohex-1-en-1-yl)-4-(2,5-difluorophenyl)pyridin-3-yl)carbamate (2.37 g, 5.39 mmol) in anhydrous dioxane (11 mL) was added 4.0 M hydrochloric acid in anhydrous dioxane (4.70 mL, 135 mmol) and the reaction mixture was stirred at ambient temperature for 16 h. To the reaction mixture was added 4.0 M hydrochloric acid in anhydrous dioxane (1.90 mL, 53.9 mmol) and the reaction mixture was stirred at 50° C. for 2 h. After cooling to ambient temperature, the mixture was diluted with saturated sodium bicarbonate (200 mL) and the aqueous phase was extracted with ethyl acetate (2×150 mL). The combined organic phase was washed with brine (150 mL), dried over anhydrous magnesium sulfate, filtered and concentrated in vacuo. Purification of the residue by column chromatography, eluting with a gradient of 10 to 100% of ethyl acetate in heptane, afforded the title compound as colorless solid (1.77 g, 97% yield): MS (ES+) m/z 340.4 (M+1).

### Step 3. Preparation of 2-(anti-4,4-difluoro-2-(methyl-d3)cyclohexyl)-4-(2,5-difluorophenyl)pyridin-3-amine

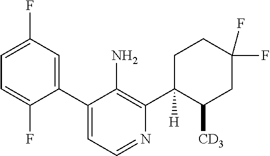 [SMILES: [2H]C([2H])([2H])[C@@H]1CC(F)(F)CC[C@@H]1c1nccc(-c2cc(F)ccc2F)c1N]

A mixture of 2-(4,4-difluoro-6-(methyl-d₃)cyclohex-1-en-1-yl)-4-(2,5-difluorophenyl)pyridin-3-amine (1.54 g, 4.55 mmol) in ethanol (45 mL) and acetic acid (0.31 mL, 5.5 mmol) was degassed with nitrogen for 10 minutes. To the reaction mixture was added ammonium formate (8.61 g, 137 mmol) and 20% palladium hydroxide on carbon (1.28 g). The reaction mixture was stirred at 80° C. for 1 h. After cooling to ambient temperature, to the reaction mixture was added ammonium formate (8.61 g, 137 mmol) and 20% palladium hydroxide on carbon (1.28 g). The reaction mixture was stirred at 80° C. for 2.5 h. After cooling to ambient temperature, the mixture was diluted with ethyl acetate (300 mL) and filtered through a bed of Celite. The filtrate was washed with potassium carbonate (100 mL), brine (100 mL), dried over anhydrous magnesium sulfate, and concentrated in vacuo. The residue was purified by column chromatography, eluting with a gradient of 5 to 15% of ethyl acetate in heptane to afford the title compound as a colorless solid (0.666 g, 43% yield): MS (ES+) m/z 342.2 (M+1).

### Step 4. Preparation of N-(2-(syn-4,4-difluoro-2-(methyl-d₃)cyclohexyl)-4-(2,5-difluorophenyl)pyridin-3-yl)-1-(difluoromethyl)-1H-pyrazole-4-carboxamide

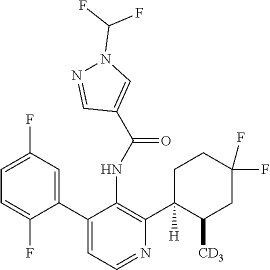 [SMILES: [2H]C([2H])([2H])[C@@H]1CC(F)(F)CC[C@@H]1c1nccc(-c2cc(F)ccc2F)c1NC(=O)c1cnn(C(F)F)c1]

To a mixture of 2-(syn-4,4-difluoro-2-(methyl-d₃)cyclohexyl)-4-(2,5-difluorophenyl)pyridin-3-amine (0.105 g, 0.308 mmol) in anhydrous tetrahydrofuran (6.20 mL) was added N,N-diisopropylethylamine (1.10 mL, 6.15 mmol), 2-chloro-1-methylpyridinium iodide (0.393 g, 1.54 mmol) and 1-(difluoromethyl)-1H-pyrazole-4-carboxylic acid (0.150 g, 0.923 mmol). The reaction mixture was stirred at 65° C. for 24 h. After cooling to ambient temperature, to the reaction mixture was added 2-chloro-1-methylpyridinium iodide (0.079 g, 0.31 mmol) and 1-(difluoromethyl)-1H-pyrazole-4-carboxylic acid (0.025 g, 0.15 mmol). The reaction mixture was stirred at 65° C. for 16 h. After cooling to ambient temperature, the reaction mixture was diluted with 1 M sodium hydroxide (50 mL) and extracted with ethyl acetate (2×100 mL). The combined organic phase was washed with saturated ammonium chloride (50 mL) and brine (50 mL), dried over anhydrous magnesium sulfate, filtered, and concentrated in vacuo. The residue was purified by reverse phase column chromatography, using a gradient of 10 to 95% of acetonitrile in water containing 0.5% formic acid as eluent, to afford the title compound as a colorless solid (0.073 g, 49% yield): ¹H NMR (400 MHz, DMSO-d₆) δ 9.94 (s, 1H), 8.68 (s, 1H), 8.61 (d, J=4.9 Hz, 1H), 8.14 (s, 1H), 7.85 (t, J=58.8 Hz, 1H), 7.36 (d, J=4.9 Hz, 1H), 7.31 (td, J=9.1, 4.5 Hz, 1H), 7.24 (tt, J=7.8, 3.9 Hz, 2H), 3.43 (s, 1H), 2.58-2.41 (m, 1H), 2.34-2.01 (m, 3H), 1.96-1.78 (m, 3H); MS (ES+) m/z 486.3 (M+1).

### Example 630

In a similar manner as described in Example 629, utilizing the appropriately substituted starting materials and intermediates, the following compounds were prepared:

| Example | Structure | Amount | MS (ES+) | |
|---|---|---|---|---|
| No. | Name | Yield | m/z | ¹H NMR |
| 630 | 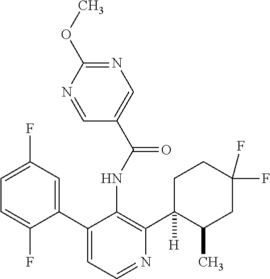 | 0.095 g 45% | 478.2 (M + 1) | (400 MHz, DMSO-d₆) δ 10.23 (s, 1H), 8.87 (s, 2H), 8.63 (d, J = 4.9 Hz, 1H), 7.39 (d, J = 5.0 Hz, 1H), 7.34 (td, J = 9.3, 4.5 Hz, 1H), 7.30- 7.21 (m, 2H), 3.97 (s, 3H), 3.48-3.42 (m, 1H), 2.46-2.38 (m, 1H), 2.33-2.17 (m, 2H), 2.12-2.05 (m, 1H), 2.01-1.79 (m, 3H) |
| | N-(2-(syn-4,4-difluoro-2-(methyl-d₃) | | | |
| | cyclohexyl)-4-(2,5-difluorophenyl)pyridin- | | | |
| | 3-yl)-2-methoxypyrimidine-5-carboxamide | | | |

### Example 631

### Synthesis of (2-(anti-4,4-difluoro-2-(methyl-d₃)cyclohexyl)-4-(2,5-difluorophenyl)pyridin-3-yl)-1-(difluoromethyl)-1H-pyrazole-4-carboxamide

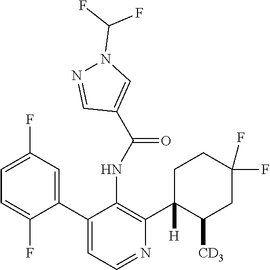 [SMILES: [2H]C([2H])([2H])[C@@H]1CC(F)(F)CC[C@H]1c1nccc(-c2cc(F)ccc2F)c1NC(=O)c1cnn(C(F)F)c1]

### Step 1. Preparation of 2-(anti-4,4-difluoro-2-(methyl-d3)cyclohexyl)-4-(2,5-difluorophenyl)pyridin-3-amine

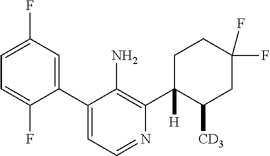 [SMILES: [2H]C([2H])([2H])[C@@H]1CC(F)(F)CC[C@H]1c1nccc(-c2cc(F)ccc2F)c1N]

A mixture of 2-(4,4-difluoro-6-(methyl-d₃)cyclohex-1-en-1-yl)-4-(2,5-difluorophenyl)pyridin-3-amine (1.54 g, 4.55 mmol) in ethanol (45 mL) and acetic acid (0.31 mL, 5.5 mmol) was degassed with nitrogen for 10 minutes. To the reaction mixture was added ammonium formate (8.61 g, 137 mmol) and 20% palladium hydroxide on carbon (1.28 g). The reaction mixture was stirred at 80° C. for 1 h. After cooling to ambient temperature, to the reaction mixture was added ammonium formate (8.61 g, 137 mmol) and 20% palladium hydroxide (1.28 g). The reaction mixture was stirred at 80° C. for 2.5 h. After cooling to ambient temperature, the mixture was diluted with ethyl acetate (300 mL) and filtered through a bed of Celite. The filtrate was washed with potassium carbonate (100 mL), brine (100 mL), dried over anhydrous magnesium sulfate, and concentrated in vacuo. The residue was purified by column chromatography, eluting with a gradient of 5 to 15% of ethyl acetate in heptane to afford the title compound as a colorless solid (0.325 g, 21% yield): MS (ES+) m/z 342.2 (M+1).

### Step 2. Preparation of N-(2-(anti-4,4-difluoro-2-(methyl-d₃)cyclohexyl)-4-(2,5-difluorophenyl)pyridin-3-yl)-1-(difluoromethyl)-1H-pyrazole-4-carboxamide

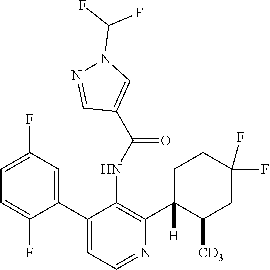 [SMILES: [2H]C([2H])([2H])[C@@H]1CC(F)(F)CC[C@H]1c1nccc(-c2cc(F)ccc2F)c1NC(=O)c1cnn(C(F)F)c1]

To a mixture of 2-(anti-4,4-difluoro-2-(methyl-d₃)cyclohexyl)-4-(2,5-difluorophenyl)pyridin-3-amine (0.105 g, 0.308 mmol) in anhydrous tetrahydrofuran (6.20 mL) was added N,N-diisopropylethylamine (1.10 mL, 6.15 mmol), 2-chloro-1-methylpyridinium iodide (0.393 g, 1.54 mmol) and 1-(difluoromethyl)-1H-pyrazole-4-carboxylic acid (0.150 g, 0.923 mmol). The reaction mixture was stirred at 65° C. for 24 h. After cooling to ambient temperature, the reaction mixture was diluted with 1 M sodium hydroxide (50 mL) and extracted with ethyl acetate (2×100 mL). The combined organic phases were washed with saturated ammonium chloride (50 mL) and brine (50 mL), dried over anhydrous magnesium sulfate, filtered, and concentrated in vacuo. The residue was purified by reverse phase column chromatography, eluting with a gradient of 10 to 95% of acetonitrile in water containing 0.5% formic acid as eluent, to afford the title compound as a colorless solid (0.064 g, 43% yield): ¹H NMR (400 MHz, DMSO-d₆) δ 9.92 (d, J=0.3 Hz, 1H), 8.69 (s, 1H), 8.62 (d, J=4.9 Hz, 1H), 8.15 (s, 1H), 7.80 (d, J=58.8 Hz, 1H), 7.34-7.20 (m, 4H), 2.79-2.77 (m, 1H), 2.27-2.22 (m, 1H), 2.08 (d, J=3.5 Hz, 2H), 1.90-1.57 (m, 4H); MS (ES+) m/z 486.4 (M+1).

### Example 632

In a similar manner as described in Example 631, utilizing the appropriately substituted starting materials and intermediates, the following compounds were prepared:

| Example | Structure | Amount | MS (ES+) | |
|---|---|---|---|---|
| No. | Name | Yield | m/z | ¹H NMR |
| 632 | 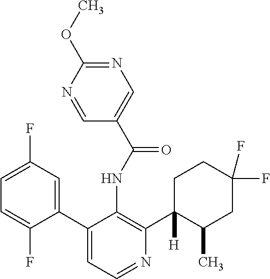 | 0.050 g 34% | 478.3 (M + 1) | (400 MHz, DMSO-d₆) δ 10.23 (s, 1H), 8.88 (s, 2H), 8.64 (d, J = 4.9 Hz, 1H), 7.34 (dq, J = 9.6, 4.7 Hz, 2H), 7.28-7.23 (m, 2H), 3.98 (s, 3H), 2.84 (t, J = 8.4 Hz, 1H), 2.27-2.21 (m, 1H), 2.08-2.05 (m, 2H), 1.95-1.61 (m, 4H) |
| | N-(2-(anti-4,4-difluoro-2-(methyl-d₃) | | | |
| | cyclohexyl)-4-(2,5-difluorophenyl)pyridin- | | | |
| | 3-yl)-2-methoxypyrimidine-5-carboxamide | | | |

### Example 633

### Synthesis of N-(2-(4,4-difluoro-6,6-bis(methyl-d₃)cyclohex-1-en-1-yl)-4-(2,5-difluorophenyl)pyridin-3-yl)-2-isopropylpyrimidine-5-carboxamide

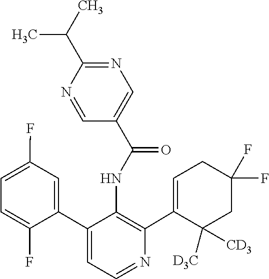 [SMILES: [2H]C([2H])([2H])C1(C([2H])([2H])[2H])CC(F)(F)CC=C1c1nccc(-c2cc(F)ccc2F)c1NC(=O)c1cnc(C(C)C)nc1]

### Step 1. Preparation of tert-butyl (2-(4,4-difluoro-6,6-bis(methyl-d₃)cyclohex-1-en-1-yl)-4-(2,5-difluorophenyl)pyridin-3-yl)carbamate

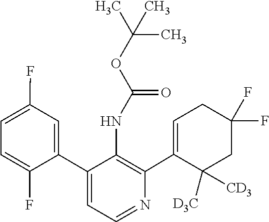 [SMILES: [2H]C([2H])([2H])C1(C([2H])([2H])[2H])CC(F)(F)CC=C1c1nccc(-c2cc(F)ccc2F)c1NC(=O)OC(C)(C)C]

A mixture of tert-butyl (2-chloro-4-(2,5-difluorophenyl)pyridin-3-yl)carbamate (3.27 g, 9.48 mmol) in dioxane (66 mL) was degassed with nitrogen for 20 minutes. To the reaction mixture was added N′-(4,4-difluoro-2-(methyl-d₃)cyclohexylidene)-4-methylbenzenesulfonohydrazide containing 30% N′-(4,4-difluoro-2,2-bis(methyl-d₃)cyclohexylidene)-4-methylbenzenesulfonohydrazide (4.74 g), tris(dibenzylideneacetone)dipalladium(0) (1.36 g, 1.48 mmol), tricyclohexylphosphine tetrafluoroborate (1.13 g, 3.06 mmol) and lithium tert-butoxide (2.77 g, 34.6 mmol). The reaction was stirred at 100° C. for 16 h. After cooling to ambient temperature, the mixture was diluted with ethyl acetate (300 mL) and filtered through a bed of Celite. The filtrate was washed with water (100 mL), brine (100 mL), dried over anhydrous magnesium sulfate, and concentrated in vacuo. The residue was purified by column chromatography, eluting with a gradient of 10 to 25% of ethyl acetate in heptane to afford the title compound as a brown solid (0.664 g, 15% yield): MS (ES+) m/z 457.4 (M+1).

### Step 2. Preparation of 2-(4,4-difluoro-6,6-bis(methyl-d₃)cyclohex-1-en-1-yl)-4-(2,5-difluorophenyl)pyridin-3-amine

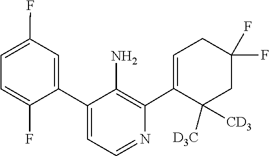 [SMILES: [2H]C([2H])([2H])C1(C([2H])([2H])[2H])CC(F)(F)CC=C1c1nccc(-c2cc(F)ccc2F)c1N]

To tert-butyl (2-(4,4-difluoro-6-(methyl-d₃)cyclohex-1-en-1-yl)-4-(2,5-difluorophenyl)pyridin-3-yl)carbamate (0.664 g, 1.45 mmol) was added 4.0 M hydrochloric acid in anhydrous dioxane (18.2 mL, 72.7 mmol). The reaction mixture was stirred at 40° C. for 16 h. After cooling to ambient temperature, the mixture was diluted with ethyl acetate (150 mL). The combined organic phase was washed with saturated potassium carbonate (50 mL), brine (50 mL), dried over anhydrous magnesium sulfate, filtered, and concentrated in vacuo. Purification of the residue by column chromatography, eluting with a gradient of 5 to 50% of ethyl acetate in heptane, afforded the title compound as an orange solid (0.779 g, 150% yield): ¹H NMR (400 MHz, CDCl₃) δ 8.07 (d, J=4.9 Hz, 1H), 7.19-7.08 (m, 3H), 6.97 (d, J=4.9 Hz, 1H), 5.63 (quintet, J=3.3 Hz, 1H), 3.81 (s, 2H), 2.72 (td, J=14.1, 3.8 Hz, 2H), 2.13 (t, J=14.9 Hz, 2H); MS (ES+) m/z 357.4 (M+1).

### Step 3. Preparation of N-(2-(4,4-difluoro-6,6-bis(methyl-d₃)cyclohex-1-en-1-yl)-4-(2,5-difluorophenyl)pyridin-3-yl)-2-isopropylpyrimidine-5-carboxamide

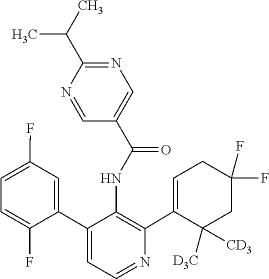 [SMILES: [2H]C([2H])([2H])C1(C([2H])([2H])[2H])CC(F)(F)CC=C1c1nccc(-c2cc(F)ccc2F)c1NC(=O)c1cnc(C(C)C)nc1]

To a mixture of 2-(4,4-difluoro-6,6-bis(methyl-d₃)cyclohex-1-en-1-yl)-4-(2,5-difluorophenyl)pyridin-3-amine (0.100 g, 0.281 mmol) in anhydrous tetrahydrofuran (5.6 mL) was added N,N-diisopropylethylamine (0.49 mL, 2.8 mmol), 2-chloro-1-methylpyridinium iodide (0.215 g, 0.841 mmol) and 2-isopropylpyrimidine-5-carboxylic acid (0.070 g, 0.42 mmol). The reaction mixture was stirred at 65° C. for 16 h. After cooling to ambient temperature, the reaction mixture was diluted with methanol (5 mL) and 5 M sodium hydroxide (1 mL). The reaction mixture was stirred at 45° C. for 15 minutes. The reaction mixture was diluted with ethyl acetate (150 mL) and the organic phase was washed with water (50 mL), saturated ammonium chloride (50 mL), brine (50 mL), dried over anhydrous magnesium sulfate, filtered, and concentrated in vacuo. The residue was purified by column chromatography, eluting with a gradient of 10 to 75% of ethyl acetate in heptane. Further purification by reverse phase column chromatography, using a gradient of 10 to 95% acetonitrile in water as eluent, afforded the title compound as colorless solid to afford the title compound as a colorless solid (0.071 g, 50% yield): ¹H NMR (400 MHz, DMSO-d₆) δ 10.13 (s, 1H), 8.80 (s, 2H), 8.63 (d, J=4.9 Hz, 1H), 7.45 (d, J=4.9 Hz, 1H), 7.37 (td, J=8.9, 4.5 Hz, 1H), 7.27 (tt, J=8.0, 4.0 Hz, 2H), 5.61-5.54 (m, 1H), 3.17 (sept, J=6.9 Hz, 1H), 2.69 (td, J=14.6, 3.3 Hz, 2H), 2.06 (t, J=15.2 Hz, 2H), 1.27 (dd, J=6.9, 0.8 Hz, 6H); ¹⁹F NMR (376 MHz, DMSO-d₆) δ −87.9, −119.1 (d, J=17 Hz), −120.6 (d, J=18 Hz); MS (ES+) m/z 505.4 (M+1).

### Example 634

### Synthesis of 3-(2,2-difluoroethoxy)-N-(4-(2,5-difluorophenyl)-2-(3-(trifluoromethyl) tetrahydro-2H-pyran-4-yl)pyridin-3-yl)isoxazole-5-carboxamide

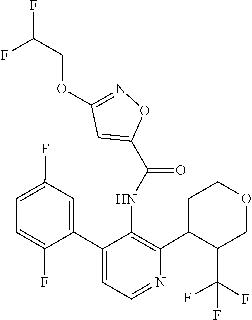 [SMILES: O=C(Nc1c(-c2cc(F)ccc2F)ccnc1C1CCOCC1C(F)(F)F)c1cc(OCC(F)F)no1]

### Step 1. Preparation of 4-(2,5-difluorophenyl)-2-(3,6-dihydro-2H-pyran-4-yl)pyridin-3-amine 2,2,2-trifluoroacetic acid salt

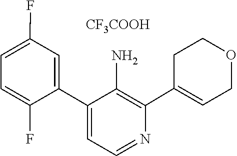 [SMILES: Nc1c(-c2cc(F)ccc2F)ccnc1C1=CCOCC1 ; "CF3COOH"]

A mixture of tert-butyl (2-chloro-4-(2,5-difluorophenyl)pyridin-3-yl)carbamate (3.00 g, 8.80 mmol) in dioxane (44 mL) and water (4.9 mL) was degassed with nitrogen for 10 minutes. To the reaction mixture was added [1,1′-bis(diphenylphosphino)ferrocene]dichloropalladium(II) dichloromethane complex (0.745 g, 0.880 mmol), 3,6-dihydro-2H-pyran-4-boronic acid pinacol ester (2.40 g, 11.4 mmol), and potassium carbonate (3.04 g, 22.0 mmol). The reaction mixture was stirred at 85° C. for 12 h. After cooling to ambient temperature, the reaction mixture was diluted with ethyl acetate (400 mL) and filtered through Celite. The organic layer was washed with saturated ammonium chloride (200 mL) and brine (200 mL), dried over anhydrous magnesium sulfate, filtered, and concentrated in vacuo. To the residue was added dichloromethane (29 mL) and 4 M hydrochloric acid in anhydrous 1,4-dioxane (33.0 mL, 132 mmol). The reaction mixture was stirred at ambient temperature for 18 h. To the reaction mixture was added saturated potassium carbonate (200 mL) and the aqueous phase was extracted with ethyl acetate (2×200 mL). The combined organic fractions were washed with brine, dried over anhydrous magnesium sulfate, filtered, and concentrated in vacuo. The residue was purified by column chromatography, eluting with a gradient of 10 to 50% of ethyl acetate in heptane, followed by suspension in diethyl ether (50 mL) and 2,2,2-trifluoroacetic acid (5 mL) and filtration, to provide the title compound as a colorless solid (2.57 g, 73% yield): ¹H NMR (400 MHz, CDCl₃) δ 8.23 (d, J=5.6 Hz, 1H), 7.36 (d, J=5.5 Hz, 1H), 7.31-7.21 (m, 2H), 7.13 (ddd, J=8.0, 5.3, 2.8 Hz, 1H), 6.37-6.34 (m, 1H), 4.37 (q, J=2.7 Hz, 2H), 4.01 (t, J=5.3 Hz, 2H), 2.62-2.56 (m, 2H); ¹⁹F NMR (376 MHz, CDCl₃) δ − 75.6, −115.8 (d, J=18 Hz), −118.9 (d, J=18 Hz); MS (ES+) m/z 289.2 (M+1).

### Step 2. Preparation of 4-(2,5-difluorophenyl)-2-(3-(trifluoromethyl)-3,6-dihydro-2H-pyran-4-yl)pyridin-3-amine and 4-(2,5-difluorophenyl)-2-(3,6-dihydro-2H-pyran-4-yl)-6-(trifluoromethyl)pyridin-3-amine

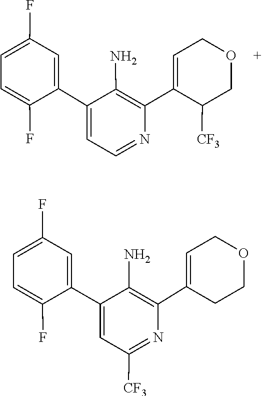 [SMILES: Nc1c(-c2cc(F)ccc2F)ccnc1C1=CCOCC1C(F)(F)F ; Nc1c(-c2cc(F)ccc2F)cc(C(F)(F)F)nc1C1=CCOCC1]

A mixture of 4-(2,5-difluorophenyl)-2-(3,6-dihydro-2H-pyran-4-yl)pyridin-3-amine 2,2,2-trifluoroacetic acid salt (1.50 g, 3.73 mmol), and anhydrous dimethylsulfoxide (37 mL) was sparged with nitrogen for 10 minutes. To the mixture was added tris(2,2′-bipyridyl)dichlororuthenium(II) hexahydrate (0.060 g, 0.093 mmol) and 5-(trifluoromethyl) dibenzothiophenium trifluoromethylsulfonate. The vial was sealed, and the reaction mixture was stirred in front of Kessil PR160L lights (440 nm) for 18 h. The reaction mixture was diluted with ethyl acetate (200 mL) and the organic phase was washed with saturated ammonium chloride (75 mL) brine (75 mL), dried over anhydrous magnesium sulfate, filtered, and concentrated in vacuo. The residue was subjected to column chromatography, eluting with a gradient of 5 to 100% of ethyl acetate in heptane to afford the title compounds as a red oil (0.391 g, mixture of regioisomers, 29% yield).

### Step 3. Preparation of 4-(2,5-difluorophenyl)-2-(3-(trifluoromethyl)tetrahydro-2H-pyran-4-yl)pyridin-3-amine formic acid salt

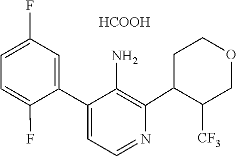 [SMILES: Nc1c(-c2cc(F)ccc2F)ccnc1C1CCOCC1C(F)(F)F ; "HCOOH"]

A mixture of 4-(2,5-difluorophenyl)-2-(3-(trifluoromethyl)-3,6-dihydro-2H-pyran-4-yl)pyridin-3-amine and 4-(2,5-difluorophenyl)-2-(3,6-dihydro-2H-pyran-4-yl)-6-(trifluoromethyl)pyridin-3-amine (0.391 g, 1.10 mmol) in ethanol (11 mL) and acetic acid (0.26 mL, 4.4 mmol) was degassed with nitrogen for 10 minutes. To the reaction mixture was added ammonium formate (0.693 g, 11.0 mmol) and 20% palladium hydroxide on carbon (0.170 g). The reaction mixture was stirred at 80° C. for 3.5 h. After cooling to ambient temperature, the mixture was diluted with ethyl acetate (200 mL) and filtered through a bed of Celite. The filtrate was washed with potassium carbonate (50 mL), brine (50 mL), dried over anhydrous magnesium sulfate, and concentrated in vacuo. The residue was purified by column chromatography, eluting with a gradient of 5 to 100% of ethyl acetate in heptane. The residue was further purified by column chromatography, eluting with a gradient of 15 to 95% of acetonitrile in water containing 0.5% formic acid, to afford the title compound as a colorless solid (0.139 g, 31% yield): ¹H NMR (400 MHz, CD₃CN) δ 8.13 (s, 0.6H), 7.95 (t, J=4.6 Hz, 1H), 7.23 (dqd, J=16.4, 8.4, 4.1 Hz, 2H), 7.12 (ddd, J=8.5, 5.5, 3.1 Hz, 1H), 6.95 (dd, J=9.9, 4.8 Hz, 1H), 4.97 (br s, 3H), 4.32 (d, J=12.5 Hz, 1H), 4.14 (d, J=10.7 Hz, 1H), 3.78 (d, J=12.4 Hz, 1H), 3.61 (td, J=11.6, 2.1 Hz, 1H), 3.52-3.50 (m, 1H), 2.97-2.95 (m, 1H), 2.72 (q, J=12.6 Hz, 1H), 1.65 (d, J=14.1 Hz, 1H); ¹⁹F NMR (376 MHz, CD₃CN) δ −62.1, −119.8, −121.5; MS (ES+) m/z 359.4 (M+1).

### Step 4. Preparation of 3-(2,2-difluoroethoxy)-N-(4-(2,5-difluorophenyl)-2-(3-(trifluoromethyl)tetrahydro-2H-pyran-4-yl)pyridin-3-yl)isoxazole-5-carboxamide

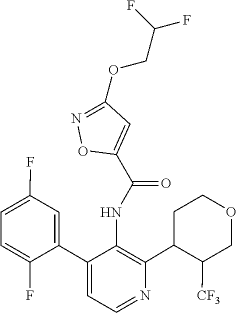 [SMILES: O=C(Nc1c(-c2cc(F)ccc2F)ccnc1C1CCOCC1C(F)(F)F)c1cc(OCC(F)F)no1]

To a mixture of 4-(2,5-difluorophenyl)-2-(3-(trifluoromethyl)tetrahydro-2H-pyran-4-yl)pyridin-3-amine formic acid salt (0.139 g, 0.344 mmol) in anhydrous tetrahydrofuran (7.0 mL) was added N,N-diisopropylethylamine (0.60 mL, 3.4 mmol), 2-chloro-1-methylpyridinium iodide (0.264 g, 1.03 mmol), and 3-(2,2-difluoroethoxy)isoxazole-5-carboxylic acid (0.080 g, 0.41 mmol). The reaction mixture was stirred at 50° C. for 18 h. After cooling to ambient temperature, to the reaction mixture was added N,N-diisopropylethylamine (0.30 mL, 1.8 mmol), 2-chloro-1-methylpyridinium iodide (0.131 g, 0.517 mmol), and 3-(2,2-difluoroethoxy)isoxazole-5-carboxylic acid (0.066 g, 0.35 mmol). The reaction mixture was stirred at 50° C. for 5.5 h. After cooling to ambient temperature, to the reaction mixture was added methanol (5 mL) and 5 M sodium hydroxide (2 mL). The reaction mixture was stirred at ambient temperature for 10 minutes. The reaction mixture was diluted with 1 M sodium hydroxide (25 mL) and extracted with ethyl acetate (100 mL). The organic phase was washed with saturated ammonium chloride (25 mL) and brine (25 mL), dried over anhydrous magnesium sulfate, filtered, and concentrated in vacuo. The residue was purified by column chromatography, eluting with a gradient of 10 to 85% of ethyl acetate in heptane. The residue was further purified by reverse phase column chromatography, eluting with a gradient of 10 to 85% of acetonitrile in water, to afford the title compound as a colorless solid (0.126 g, 69% yield): ¹H NMR (400 MHz, DMSO-d₆) δ 10.66 (s, 1H), 8.66 (d, J=4.9 Hz, 1H), 7.42 (d, J=4.9 Hz, 1H), 7.28 (ddtd, J=33.6, 16.7, 8.2, 4.1 Hz, 3H), 6.96 (s, 1H), 6.41 (tt, J=53.9, 3.1 Hz, 1H), 4.56 (td, J=15.0, 3.1 Hz, 2H), 4.22 (dd, J=12.3, 2.2 Hz, 1H), 4.11-4.08 (m, 1H), 3.74 (s, 1H), 3.64 (d, J=11.6 Hz, 1H), 3.57-3.52 (m, 1H), 2.86 (s, 1H), 2.67-2.54 (m, 1H), 1.59 (d, J=13.1 Hz, 1H); MS (ES+) m/z 534.2 (M+1).

### Example 635

### Synthesis of N-(4-(2,5-difluorophenyl)-2-(tetrahydro-2H-pyran-4-yl)-6-(trifluoromethyl)pyridin-3-yl)-2-isopropylpyrimidine-5-carboxamide

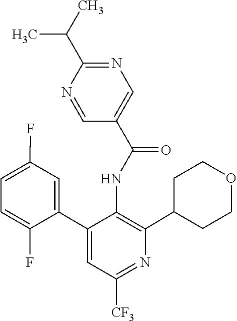 [SMILES: CC(C)c1ncc(C(=O)Nc2c(-c3cc(F)ccc3F)cc(C(F)(F)F)nc2C2CCOCC2)cn1]

### Step 1. Preparation of 4-(2,5-difluorophenyl)-2-(tetrahydro-2H-pyran-4-yl)-6-(trifluoromethyl)pyridin-3-amine

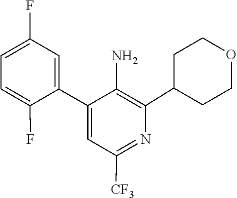 [SMILES: Nc1c(-c2cc(F)ccc2F)cc(C(F)(F)F)nc1C1CCOCC1]

A mixture of 4-(2,5-difluorophenyl)-2-(3-(trifluoromethyl)-3,6-dihydro-2H-pyran-4-yl)pyridin-3-amine and 4-(2,5-difluorophenyl)-2-(3,6-dihydro-2H-pyran-4-yl)-6-(trifluoromethyl)pyridin-3-amine (0.391 g, 1.10 mmol) in ethanol (11 mL) and acetic acid (0.26 mL, 4.4 mmol) was degassed with nitrogen for 10 minutes. To the reaction mixture was added ammonium formate (0.693 g, 11.0 mmol) and 20% palladium hydroxide on carbon (0.170 g). The reaction mixture was stirred at 80° C. for 3.5 h. After cooling to ambient temperature, the mixture was diluted with ethyl acetate (200 mL) and filtered through a bed of Celite. The filtrate was washed with potassium carbonate (50 mL) and brine (50 mL), dried over anhydrous magnesium sulfate, and concentrated in vacuo. The residue was purified by column chromatography, eluting with a gradient of 5 to 100% of ethyl acetate in heptane. The residue was further purified by column chromatography, eluting with a gradient of 15 to 95% of acetonitrile in water containing 0.5% formic acid, to afford the title compound as a colorless solid (0.063 g, 16% yield): ¹H NMR (400 MHz, CD₃CN) δ 7.32 (s, 1H), 7.30-7.21 (m, 2H), 7.16 (ddd, J=8.7, 5.6, 3.1 Hz, 1H), 4.70 (s, 2H), 4.01 (dd, J=11.1, 3.8 Hz, 2H), 3.55 (td, J=11.8, 2.0 Hz, 2H), 3.08 (tt, J=11.3, 3.8 Hz, 1H), 1.87 (td, J=12.1, 4.0 Hz, 2H), 1.77-1.73 (m, 2H); ¹⁹F NMR (376 MHz, CD₃CN) δ −66.9, −119.7, −121.7; MS (ES+) m/z 359.4 (M+1).

### Step 2. Preparation of N-(4-(2,5-difluorophenyl)-2-(tetrahydro-2H-pyran-4-yl)-6-(trifluoromethyl)pyridin-3-yl)-2-isopropylpyrimidine-5-carboxamide

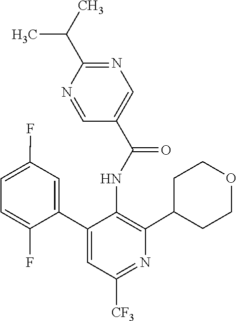 [SMILES: CC(C)c1ncc(C(=O)Nc2c(-c3cc(F)ccc3F)cc(C(F)(F)F)nc2C2CCOCC2)cn1]

To a mixture of 4-(2,5-difluorophenyl)-2-(tetrahydro-2H-pyran-4-yl)-6-(trifluoromethyl)pyridin-3-amine (0.063 g, 0.18 mmol) in anhydrous tetrahydrofuran (3.5 mL) was added N,N-diisopropylethylamine (0.31 mL, 1.8 mmol), 2-chloro-1-methylpyridinium iodide (0.135 g, 0.529 mmol), and 2-isopropylpyrimidine-5-carboxylic acid (0.044 g, 0.26 mmol). The reaction was stirred at 50° C. for 18 h. After cooling to ambient temperature, to the reaction mixture was added N,N-diisopropylethylamine (0.15 mL, 0.84 mmol), 2-chloro-1-methylpyridinium iodide (0.090 g, 0.35 mmol), and 2-isopropylpyrimidine-5-carboxylic acid (0.029 g, 0.18 mmol). The reaction was stirred at 50° C. for 6 h. After cooling to ambient temperature, the reaction mixture was diluted with methanol (5 mL) and 5 M sodium hydroxide (2 mL). The reaction mixture was stirred at ambient temperature for 30 minutes. The reaction mixture was diluted with 1 M sodium hydroxide (25 mL) and extracted with ethyl acetate (100 mL). The organic phase was washed with saturated ammonium chloride (25 mL), brine (25 mL), dried over anhydrous magnesium sulfate, filtered, and concentrated in vacuo. The residue was purified by column chromatography, eluting with a gradient of 15 to 100% of ethyl acetate in heptane, to afford the title compound as a colorless solid (0.049 g, 55% yield): ¹H NMR (400 MHz, DMSO-d₆) δ 10.63 (s, 1H), 8.96 (s, 2H), 7.92 (s, 1H), 7.34 (dtt, J=26.1, 8.6, 4.3 Hz, 3H), 3.93 (dd, J=10.9, 3.3 Hz, 2H), 3.44 (s, 3H), 3.20 (sept, J=6.9 Hz, 1H), 1.87 (q, J=11.5 Hz, 2H), 1.66 (d, J=12.5 Hz, 2H), 1.29 (d, J=6.9 Hz, 6H); ¹⁹F NMR (376 MHz, DMSO-d₆) δ −66.4, −118.5 (d, J=18 Hz), −120.1 (d, J=18 Hz); MS (ES+) m/z 507.2 (M+1).

### Example 636

### Synthesis of N-(2-(3-(difluoromethyl)tetrahydro-2H-pyran-4-yl)-4-(2,5-difluorophenyl)pyridin-3-yl)-2-isopropylpyrimidine-5-carboxamide

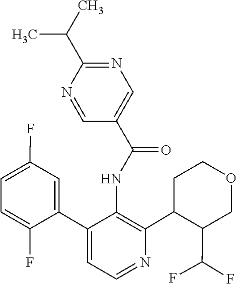 [SMILES: CC(C)c1ncc(C(=O)Nc2c(-c3cc(F)ccc3F)ccnc2C2CCOCC2C(F)F)cn1]

### Step 1. Preparation of 2-(3-(difluoromethyl)tetrahydro-2H-pyran-4-yl)-4-(2,5-difluorophenyl)pyridin-3-amine

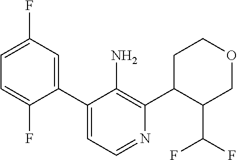 [SMILES: Nc1c(-c2cc(F)ccc2F)ccnc1C1CCOCC1C(F)F]

A mixture of 4-(2,5-difluorophenyl)-2-(3,6-dihydro-2H-pyran-4-yl)pyridin-3-amine 2,2,2-trifluoroacetic acid salt (, zinc(II) difluoromethanesulfinate (0.360 g, 2.45 mmol), and tris[2-phenylpyridinato-C²,N]iridium(III) (0.002 g, 0.003 mmol) in dimethylsulfoxide (3.1 mL) was degassed with nitrogen for 10 minutes. The vial was sealed, and the reaction mixture was stirred in front of Kessil PR160L lights (440 nm) for 22 h. The reaction mixture was diluted with ethyl acetate (75 mL) and the organic phase was washed with saturated ammonium chloride (25 mL) and brine (25 mL), dried over anhydrous magnesium sulfate, filtered, and concentrated in vacuo. The residue was purified by reverse phase column chromatography, eluting with a gradient of 5 to 95% of acetonitrile in water containing 0.5% formic acid, to afford the title compounds as a colorless oil (0.064 g, containing ˜73% 4-(2,5-difluorophenyl)-2-(3,6-dihydro-2H-pyran-4-yl)pyridin-3-amine by LCMS).

### Step 2. Preparation of N-(2-(3-(difluoromethyl)tetrahydro-2H-pyran-4-yl)-4-(2,5-difluorophenyl)pyridin-3-yl)-2-isopropylpyrimidine-5-carboxamide

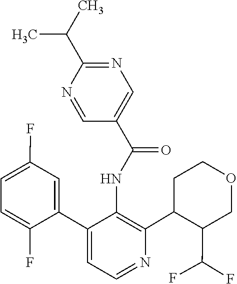 [SMILES: CC(C)c1ncc(C(=O)Nc2c(-c3cc(F)ccc3F)ccnc2C2CCOCC2C(F)F)cn1]

To a mixture of 4-(2,5-difluorophenyl)-2-(tetrahydro-2H-pyran-4-yl)-6-(trifluoromethyl)pyridin-3-amine (0.063 g, 0.18 mmol) in anhydrous tetrahydrofuran (3.5 mL) was added N,N-diisopropylethylamine (0.33 mL, 1.9 mmol), 2-chloro-1-methylpyridinium iodide (0.144 g, 0.564 mmol), and 2-isopropylpyrimidine-5-carboxylic acid (0.047 g, 0.28 mmol). The reaction mixture was stirred at 50° C. for 16 h. After cooling to ambient temperature, the reaction mixture was diluted with 1 M sodium hydroxide (25 mL) and extracted with ethyl acetate (75 mL). The organic phase was washed with saturated ammonium chloride (25 mL), brine (25 mL), dried over anhydrous magnesium sulfate, filtered, and concentrated in vacuo. The residue was purified by column chromatography, eluting with a gradient of 5 to 100% of ethyl acetate in heptane. The residue was diluted with 3 mL of methanol and 5 M NaOH (0.5 mL) was added. The reaction mixture was stirred at ambient temperature for 10 minutes. The reaction mixture was diluted with saturated potassium carbonate (25 mL) and the aqueous phase was extracted with ethyl acetate (2×50 mL). The combined organic phase was dried over anhydrous magnesium sulfate, filtered, and concentrated in vacuo. The residue was purified by column chromatography, eluting with a gradient of 20 to 100% of ethyl acetate in heptane, to afford the title compound as a colorless solid (0.005 g, 5% yield): ¹H NMR (400 MHz, DMSO-d₆) δ 10.39 (s, 1H), 8.94 (s, 2H), 8.63 (d, J=4.9 Hz, 1H), 7.42 (d, J=4.9 Hz, 1H), 7.36 (td, J=9.2, 4.6 Hz, 1H), 7.26 (dtd, J=12.7, 8.6, 3.8 Hz, 2H), 6.13 (td, J=56.2, 6.1 Hz, 1H), 4.14 (d, J=11.4 Hz, 1H), 3.97 (d, J=10.2 Hz, 1H), 3.74-3.51 (m, 4H), 3.19 (sept, J=6.9 Hz, 1H), 1.62 (d, J=13.8 Hz, 1H), 1.28 (d, J=6.9 Hz, 6H), 1.23 (s, 1H); MS (ES+) m/z 489.2 (M+1).

### Example 637

In a similar manner as described in examples disclosed herein, utilizing the appropriately substituted starting materials and intermediates, the following compounds were prepared:

| Example | Structure | Amount | MS (ES+) | |
|---|---|---|---|---|
| No. | Name | Yield | m/z | ¹H NMR |
| 637 | 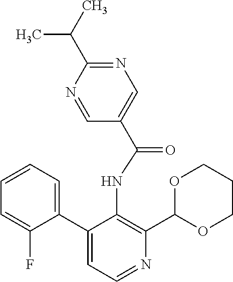 [SMILES: CC(C)c1ncc(C(=O)Nc2c(-c3ccccc3F)ccnc2C2OCCCO2)cn1] | 0.071 g 50% | 423.2 (M + 1) | (400 MHz, DMSO-d₆) δ 10.25 (s, 1H), 8.97 (s, 2H), 8.61 (d, J = 4.9 Hz, 1H), 7.52-7.51 (m, 1H), 7.43-7.37 (m, 2H), 7.30-7.26 (m, 1H), 7.21 (td, J = 7.5, 1.1 Hz, 1H), 5.77 (s, 1H), 4.10 (dd, J = 11.0, 4.7 Hz, 2H), 3.92-3.87 (m, 2H), 3.18 (sept, J = 6.9 Hz, 1H), 1.95-1.83 (m, 1H), 1.40 (d, J = 13.4 Hz, 1H), 1.28 (d, J = 6.9 Hz, 6H) |
| | N-(2-(1,3-dioxan-2-yl)-4-(2- | | | |
| | fluorophenyl)pyridin-3-yl)-2- | | | |
| | isopropylpyrimidine-5-carboxamide | | | |

### Example 638

### Synthesis of N-(3-(4,4-difluorocyclohexyl)-5-(2,5-difluorophenyl)pyridin-4-yl)-5-fluoro-6-methoxynicotinamide

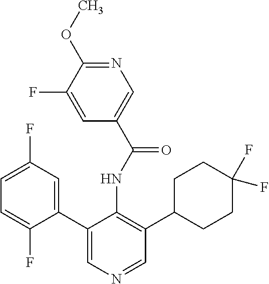 [SMILES: COc1ncc(C(=O)Nc2c(-c3cc(F)ccc3F)cncc2C2CCC(F)(F)CC2)cc1F]

### Step 1. Preparation of 3-bromo-5-(2,5-difluorophenyl)pyridin-4-amine

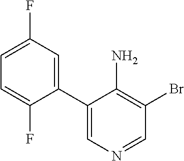 [SMILES: Nc1c(Br)cncc1-c1cc(F)ccc1F]

A mixture of 4-amino-3,5-dibromopyridine (3.00 g, 11.9 mmol) in 1,2-dimethoxyethane (74 mL) and water (36 mL), 2,5-difluorophenylboronic acid (2.07 g, 13.1 mmol), and sodium carbonate (3.29 g, 23.8 mmol) was degassed with nitrogen for 10 minutes. To the reaction mixture was added trans-dichlorobis(triphenylphosphine)palladium (II) (0.418 g, 0.600 mmol). The reaction was stirred at 90° C. for 2.5 h. After cooling to ambient temperature, the reaction mixture was diluted with water (200 mL) and the aqueous phase was extracted with ethyl acetate (3×200 mL). The combined organic phases were dried over anhydrous magnesium sulfate, filtered, and concentrated in vacuo. The residue was purified by column chromatography, eluting with a gradient of 5 to 75% of ethyl acetate in heptane, to afford the title compound as a colorless solid (0.862 g, 25% yield): MS (ES+) m/z 285.2 (M+1), 287.2 (M+1).

### Step 2. Preparation of 3-(4,4-difluorocyclohex-1-en-1-yl)-5-(2,5-difluorophenyl)pyridin-4-amine

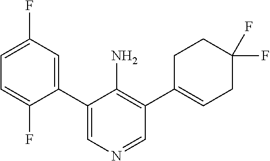 [SMILES: Nc1c(C2=CCC(F)(F)CC2)cncc1-c1cc(F)ccc1F]

A mixture of 3-bromo-5-(2,5-difluorophenyl)pyridin-4-amine (0.862 g, 3.02 mmol) in dioxane (30 mL) and water (7.6 mL) was degassed with nitrogen for 10 minutes. To the reaction mixture was added sodium carbonate (2.09 g, 15.1 mmol), 2-(4,4-difluorocyclohex-1-en-1-yl)-4,4,5,5-tetramethyl-1,3,2-dioxaborolane (0.885 g, 3.63 mmol), and tetrakis(triphenylphosphine)palladium(0) (0.699 g, 0.604 mmol). The reaction was stirred at 100° C. for 18 h. After cooling to ambient temperature, to the reaction mixture was added ethyl acetate (200 mL) and the reaction mixture was filtered through a bed of Celite. The combined filtrate was washed with saturated ammonium chloride (2×75 mL), dried with magnesium sulfate, filtered, and concentrated in vacuo. The residue was purified by column chromatography, eluting with a gradient of 5 to 100% of ethyl acetate in heptane, to afford the title compound as a pink oil (0.826 g, 85% yield): MS (ES+) m/z 323.2 (M+1).

### Step 3. Preparation of 3-(4,4-difluorocyclohexyl)-5-(2,5-difluorophenyl)pyridin-4-amine acetic acid salt

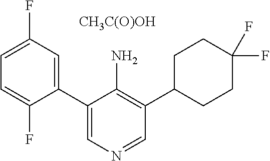

A mixture of 3-(4,4-difluorocyclohex-1-en-1-yl)-5-(2,5-difluorophenyl)pyridin-4-amine (0.826 g, 2.56 mmol) in methanol (13.0 mL), ethyl acetate (13.0 mL) was degassed with nitrogen for 10 minutes. To the reaction mixture was 10% palladium on carbon (0.273 g). The reaction mixture was degassed with hydrogen for 10 minutes. The reaction mixture was stirred at ambient temperature under an atmosphere of hydrogen for 18 h. To the reaction mixture was added acetic acid (0.44 mL, 7.7 mmol) and 10% palladium on carbon (0.273 g). The reaction mixture was degassed with hydrogen for 10 minutes. The reaction mixture was stirred at ambient temperature under an atmosphere of hydrogen for 5 h. The reaction mixture was diluted with ethyl acetate (150 mL) and filtered through a bed of Celite. The filtrate was concentrated in vacuo. The residue was purified by column chromatography, eluting with a gradient of 5 to 100% of ethyl acetate in heptane, to afford the title compound as a colorless solid (0.579 g, 60% yield): MS (ES+) m/z 325.2 (M+1).

### Step 4. Preparation of N-(3-(4,4-difluorocyclohexyl)-5-(2,5-difluorophenyl)pyridin-4-yl)-5-fluoro-6-methoxynicotinamide

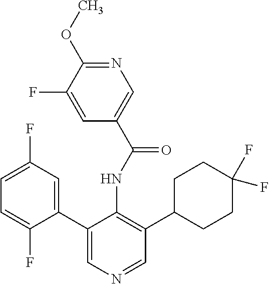 [SMILES: COc1ncc(C(=O)Nc2c(-c3cc(F)ccc3F)cncc2C2CCC(F)(F)CC2)cc1F]

To a mixture of 3-(4,4-difluorocyclohexyl)-5-(2,5-difluorophenyl)pyridin-4-amine acetic acid salt (0.100 g, 0.260 mmol) in anhydrous tetrahydrofuran (3.0 mL) was added N,N-diisopropylethylamine (0.54 mL, 3.1 mmol), 2-chloro-1-methylpyridinium iodide (0.158 g, 0.617 mmol), and 5-fluoro-6-methoxynicotinic acid (0.063 g, 0.37 mmol). The reaction mixture was stirred at 65° C. for 3 h. After cooling to ambient temperature, the reaction mixture was diluted with ethyl acetate (75 mL) and the organic phase was washed with saturated ammonium chloride (3×25 mL), dried over anhydrous magnesium sulfate, filtered, and concentrated in vacuo. The residue was purified by column chromatography, eluting with a gradient of 15 to 100% of ethyl acetate in heptane, to afford the title compound as a colorless solid (0.033 g, 18% yield): ¹H NMR (400 MHz, DMSO-d₆) δ 10.21 (s, 1H), 8.70 (s, 1H), 8.49 (s, 1H), 8.37 (d, J=1.4 Hz, 1H), 7.93-7.90 (m, 1H), 7.31 (td, J=9.4, 4.6 Hz, 1H), 7.27-7.20 (m, 2H), 4.00 (s, 3H), 3.00 (t, J=11.8 Hz, 1H), 2.14-2.09 (m, 2H), 1.99-1.77 (m, 6H); MS (ES+) m/z 478.2 (M+1).

### Example 639-666

Single enantiomers of the given examples were obtained by chiral SFC using the conditions specified:

| Example | Structure Name | SFC | Retention time (Rₜ), ee; ¹H |
|---|---|---|---|
| No. | P1 or P2 | Conditions | NMR; MS (ES+) m/z |
| 639 | 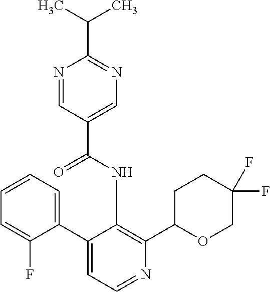 [SMILES: CC(C)c1ncc(C(=O)Nc2c(-c3ccccc3F)ccnc2C2CCC(F)(F)CO2)cn1] | Chiralpak AD-3 column (50 mm × 4.6 mm, 3 μm) eluting with 5 to 40% methanol containing 0.05% diethyl amine in supercritical carbon dioxide | 0.957 min, 99%; (400 MHz, CDCl₃) δ 8.94 (s, 3H), 8.56 (d, J = 5.2 Hz, 1H), 7.46-7.32 (m, 3H), 7.24-7.19 (m, 1H), 7.13 (t, J = 9.2 Hz, 1H), 5.06-4.97 (m, 1H), 4.13 (td, J = 23.2, 6.2, 2.6 Hz, 1H), 3.83-3.67 (m, 1H), 3.28 (q, J = 6.8 Hz, 1H), 2.40- 2.28 (m, 1H), 2.25-2.07 (m, 3H), 1.37 (d, J = 6.8 Hz, 6H); 457.2 (M + 1) |
| | N-(2-(5,5- | | |
| | difluorotetrahydro-2H- | | |
| | pyran-2-yl)-4-(2- | | |
| | fluorophenyl)pyridin-3-yl)- | | |
| | 2-isopropylpyrimidine-5- | | |
| | carboxamide | | |
| | P1 | | |
| 640 | 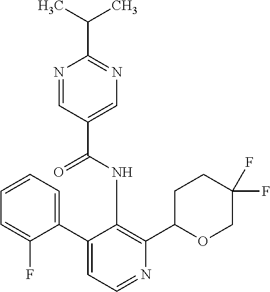 [SMILES: CC(C)c1ncc(C(=O)Nc2c(-c3ccccc3F)ccnc2C2CCC(F)(F)CO2)cn1] | Chiralpak AD-3 column (50 mm × 4.6 mm, 3 μm) eluting with 5 to 40% methanol containing 0.05% diethyl amine in supercritical carbon dioxide | 1.16 min, 99%; (400 MHz, CDCl₃) δ 8.97-8.85 (m, 3H), 8.56 (d, J = 5.0 Hz, 1H), 7.43- 7.30 (m, 3H), 7.24-7.17 (m, 1H), 7.12 (t, 9.2 Hz, 1H), 4.94-4.85 (m, 1H), 4.17-4.05 (m, 1H), 3.80-3.65 (m, 1H), 3.28 (t, J = 6.8, 13.8 Hz, 1H), 2.41-2.26 (m, 1H), 2.24-1.99 (m, 3H), 1.36 (d, J = 6.8 Hz, 6H); 457.1 (M + 1) |
| | N-(2-(5,5- | | |
| | difluorotetrahydro-2H- | | |
| | pyran-2-yl)-4-(2- | | |
| | fluorophenyl)pyridin-3-yl)- | | |
| | 2-isopropylpyrimidine-5- | | |
| | carboxamide | | |
| | P2 | | |
| 641 | 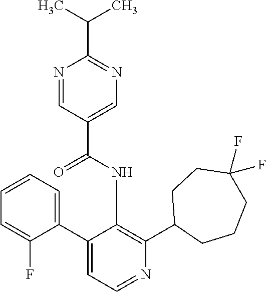 [SMILES: CC(C)c1ncc(C(=O)Nc2c(-c3ccccc3F)ccnc2C2CCCC(F)(F)CC2)cn1] | Chiralpak OJ column (250 mm × 30 mm, 10 μm) eluting with 25% methanol containing 0.1% ammonium hydroxide in supercritical carbon dioxide | 0.816 min, 99%; (400 MHz, CDCl₃) δ 8.91 (s, 2H), 8.68 (d, J = 5.2 Hz, 1H), 7.56-7.47 (m, 1H), 7.45-7.32 (m, 2H), 7.26- 7.15 (m, 3H), 3.29 (quin, J = 6.8 Hz, 1H), 3.22-3.08 (m, 1H), 2.50-2.32 (m, 1H), 2.20-1.88 (m, 7H), 1.66-1.50 (m, 2H), 1.37 (d, J = 6.8 Hz, 6H); 469.4 (M + 1) |
| | N-(2-(4,4- | | |
| | difluorocycloheptyl)-4-(2- | | |
| | fluorophenyl)pyridin-3-yl)- | | |
| | 2-isopropylpyrimidine-5- | | |
| | carboxamide | | |
| | P1 | | |
| 642 | 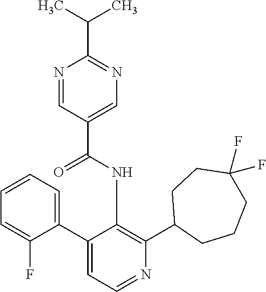 [SMILES: CC(C)c1ncc(C(=O)Nc2c(-c3ccccc3F)ccnc2C2CCCC(F)(F)CC2)cn1] | Chiralpak OJ column (250 mm × 30 mm, 10 μm) eluting with 25% methanol containing 0.1% ammonium hydroxide in supercritical carbon dioxide | 1.193 min, 99%; (400 MHz, CDCl₃) δ 8.91 (s, 2H), 8.68 (d, J = 4.8 Hz, 1H), 7.49 (d, J = 1.6 Hz, 1H), 7.45-7.33 (m, 2H), 7.26-7.16 (m, 3H), 3.29 (td, J = 6.8, 13.6 Hz, 1H), 3.14 (dt, J = 5.2, 9.2 Hz, 1H), 2.50-2.33 (m, 1H), 2.22-1.90 (m, 7H), 1.59- 1.53 (m, 2H), 1.37 (d, J = 6.8 Hz, 6H); 469.4 (M + 1) |
| | N-(2-(4,4- | | |
| | difluorocycloheptyl)-4-(2- | | |
| | fluorophenyl)pyridin-3-yl)- | | |
| | 2-isopropylpyrimidine-5- | | |
| | carboxamide | | |
| | P2 | | |
| 643 | 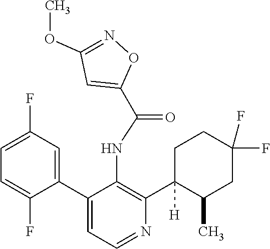 [SMILES: COc1cc(C(=O)Nc2c(-c3cc(F)ccc3F)ccnc2[C@H]2CCC(F)(F)C[C@H]2C)on1] | Chiralcel OJ column (250 mm × 30 mm, 10 μm) eluting with 20% methanol in supercritical carbon dioxide | 0.718 min, 99%; (400 MHz, CDCl₃) δ 8.66 (d, J = 4.8 Hz, 1H), 7.85 (s, 1H), 7.21 (d, J = 4.8 Hz, 1H), 7.16-7.00 (m, 3H), 6.49 (s, 1H), 4.02 (s, 3H), 3.34-3.25 (m, 1H), 2.70-2.38 (m, 2H), 2.38-2.23 (m, 1H), 2.21-2.06 (m, 1H), 2.06-1.83 (m, 3H), 0.84 (d, J = 7.2 Hz, 3H); 464.2 (M + 1) |
| | N-(2-(syn-4,4-difluoro-2- | | |
| | methylcyclohexyl)-4-(2,5- | | |
| | difluorophenyl)pyridin-3- | | |
| | yl)-3-methoxyisoxazole-5- | | |
| | carboxamide | | |
| | P1 | | |
| 644 | 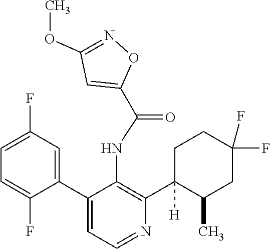 [SMILES: COc1cc(C(=O)Nc2c(-c3cc(F)ccc3F)ccnc2[C@H]2CCC(F)(F)C[C@H]2C)on1] | Chiralpak OJ column (250 mm × 30 mm, 10 μm) eluting with 20% methanol in supercritical carbon dioxide | 1.014 min, 99%; (400 MHz, CDCl₃) δ 8.66 (d, J = 4.8 Hz, 1H), 7.84 (s, 1H), 7.21 (d, J = 4.8 Hz, 1H), 7.15-7.01 (m, 3H), 6.49 (s, 1H), 4.02 (s, 3H), 3.34-3.25 (m, 1H), 2.69-2.40 (m, 2H), 2.38-2.22 (m, 1H), 2.21-2.07 (m, 1H), 2.06-1.85 (m, 3H), 0.84 (d, J = 7.2 Hz, 3H); 464.1 (M + 1) |
| | N-(2-(syn-4,4-difluoro-2- | | |
| | methylcyclohexyl)-4-(2,5- | | |
| | difluorophenyl)pyridin-3- | | |
| | yl)-3-methoxyisoxazole-5- | | |
| | carboxamide | | |
| | P2 | | |
| 645 | 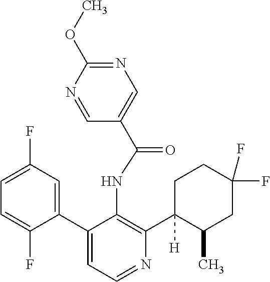 [SMILES: COc1ncc(C(=O)Nc2c(-c3cc(F)ccc3F)ccnc2[C@H]2CCC(F)(F)C[C@H]2C)cn1] | Chiralpak OJ column (250 mm × 30 mm, 10 μm) eluting with 30% methanol containing 0.1% ammonium hydroxide in supercritical carbon dioxide | 1.196 min, 99%; (400 MHz, CDCl₃) δ 8.81 (s, 2H), 8.67 (d, J = 4.8 Hz, 1H), 7.42 (s, 1H), 7.22 (d, J = 4.8 Hz, 1H), 7.16- 7.00 (m, 3H), 4.08 (s, 3H), 3.40-3.26 (m, 1H), 2.74-2.40 (m, 2H), 2.38-2.24 (m, 1H), 2.23-2.09 (m, 1H), 2.07-1.78 (m, 3H), 0.86 (d, J = 7.2 Hz, 3H); MS (ES+) m/z 475.3 (M + 1) |
| | N-(2-(syn-4,4-difluoro-2- | | |
| | methylcyclohexyl)-4-(2,5- | | |
| | difluorophenyl)pyridin-3- | | |
| | yl)-2-methoxypyrimidine-5- | | |
| | carboxamide | | |
| | P1 | | |
| 646 | 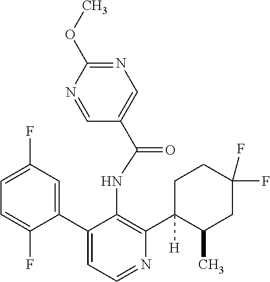 [SMILES: COc1ncc(C(=O)Nc2c(-c3cc(F)ccc3F)ccnc2[C@H]2CCC(F)(F)C[C@H]2C)cn1] | Chiralpak OJ column (250 mm × 30 mm, 10 μm) eluting with 30% methanol containing 0.1% ammonium hydroxide in supercritical carbon dioxide | 1.489 min, 99%; (400 MHz, CDCl₃) δ 8.81 (s, 2H), 8.67 (d, J = 4.8 Hz, 1H), 7.42 (s, 1H), 7.22 (d, J = 4.8 Hz, 1H), 7.16- 7.00 (m, 3H), 4.08 (s, 3H), 3.38-3.27 (m, 1H), 2.74-2.40 (m, 2H), 2.31 (d, J = 4.4 Hz, 1H), 2.22-2.09 (m, 1H), 2.07- 1.81 (m, 3H), 0.86 (d, J = 7.2 Hz, 3H); 475.4 (M + 1) |
| | N-(2-(syn-4,4-difluoro-2- | | |
| | methylcyclohexyl)-4-(2,5- | | |
| | difluorophenyl)pyridin-3- | | |
| | yl)-2-methoxypyrimidine-5- | | |
| | carboxamide | | |
| | P2 | | |
| 647 | 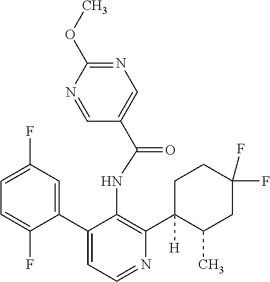 [SMILES: COc1ncc(C(=O)Nc2c(-c3cc(F)ccc3F)ccnc2[C@H]2CCC(F)(F)C[C@@H]2C)cn1] | Chiralpak OJ column (250 mm × 30 mm, 10 μm) eluting with 20% methanol containing 0.1% ammonium hydroxide in supercritical carbon dioxide | 0.984 min, 99%; (400 MHz, CDCl₃) δ 8.83 (s, 2H), 8.72 (d, J = 4.8 Hz, 1H), 7.44 (s, 1H), 7.21 (d, J = 4.8 Hz, 1H), 7.16- 7.01 (m, 3H), 4.09 (s, 3H), 2.68-2.55 (m, 1H), 2.51-2.35 (m, 1H), 2.23 (d, J = 2.8 Hz, 2H), 2.16-2.04 (m, 1H), 2.04- 1.93 (m, 1H), 1.85-1.64 (m, 1H), 1.59-1.42 (m, 1H), 0.75 (d, J = 6.6 Hz, 3H); 475.1 (M + 1) |
| | N-(2-(anti-4,4-difluoro-2- | | |
| | methylcyclohexyl)-4-(2,5- | | |
| | difluorophenyl)pyridin-3- | | |
| | yl)-2-methoxypyrimidine-5- | | |
| | carboxamide | | |
| | P1 | | |
| 648 | 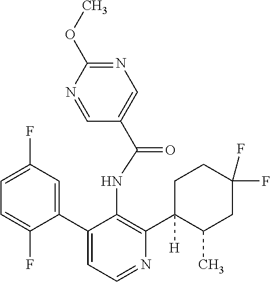 [SMILES: COc1ncc(C(=O)Nc2c(-c3cc(F)ccc3F)ccnc2[C@H]2CCC(F)(F)C[C@@H]2C)cn1] | Chiralpak OJ column (250 mm × 30 mm, 10 μm) eluting with 20% methanol containing 0.1% ammonium hydroxide in supercritical carbon dioxide | 1.164 min, 98%; (400 MHz, CDCl₃) δ 8.83 (s, 2H), 8.72 (d, J = 4.8 Hz, 1H), 7.42 (s, 1H), 7.21 (d, J = 4.8 Hz, 1H), 7.16- 6.99 (m, 3H), 4.09 (s, 3H), 2.68-2.57 (m, 1H), 2.43 (dd, J = 5.2, 11.2 Hz, 1H), 2.23 (d, J = 2.8 Hz, 2H), 2.17-2.05 (m, 1H), 2.04-1.94 (m, 1H), 1.85-1.64 (m, 1H), 1.58-1.43 (m, 1H), 0.75 (d, J = 6.4 Hz, 3H); 475.1 (M + 1) |
| | N-(2-(anti-4,4-difluoro-2- | | |
| | methylcyclohexyl)-4-(2,5- | | |
| | difluorophenyl)pyridin-3- | | |
| | yl)-2-methoxypyrimidine-5- | | |
| | carboxamide | | |
| | P2 | | |
| 649 | 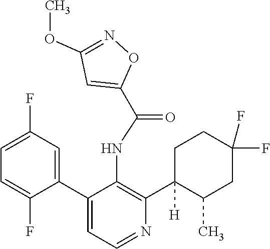 [SMILES: COc1cc(C(=O)Nc2c(-c3cc(F)ccc3F)ccnc2[C@H]2CCC(F)(F)C[C@@H]2C)on1] | Chiralpak IC column (250 mm × 30 mm, 5 μm) eluting with 15% ethanol containing 0.1% ammonium hydroxide in supercritical carbon dioxide | 0.582 min, 98%; (400 MHz, DMSO-d₆) δ 10.62 (s, 1H), 8.64 (d, J = 4.8 Hz, 1H), 7.39-7.20 (m, 4H), 6.83 (s, 1H), 3.94 (s, 3H), 2.83-2.71 (m, 1H), 2.33-2.18 (m, 1H), 2.16-2.00 (m, 2H), 1.96-1.57 (m, 4H), 0.62 (d, J = 6.0 Hz, 3H); 464.3 (M+ 1) |
| | N-(2-(anti-4,4-difluoro-2- | | |
| | methylcyclohexyl)-4-(2,5- | | |
| | difluorophenyl)pyridin-3- | | |
| | yl)-3-methoxyisoxazole-5- | | |
| | carboxamide | | |
| | P1 | | |
| 650 | 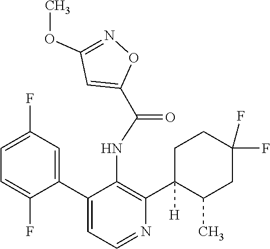 [SMILES: COc1cc(C(=O)Nc2c(-c3cc(F)ccc3F)ccnc2[C@H]2CCC(F)(F)C[C@@H]2C)on1] | Chiralpak 1C column (250 mm × 30 mm, 5 μm) eluting with 15% ethanol containing 0.1% ammonium hydroxide in supercritical carbon dioxide | 0.770 min, 98%; (400 MHz, DMSO-d₆) δ 10.62 (s, 1H), 8.64 (d, J = 4.8 Hz, 1H), 7.40-7.20 (m, 4H), 6.83 (s, 1H), 3.94 (s, 3H), 2.85-2.71 (m, 1H), 2.31- 2.20 (m, 1H), 2.16-2.01 (m, 2H), 1.94-1.58 (m, 4H), 0.62 (d, J = 6.0 Hz, 3H); 464.3 (M + 1) |
| | N-(2-(anti-4,4-difluoro-2- | | |
| | methylcyclohexyl)-4-(2,5- | | |
| | difluorophenyl)pyridin-3- | | |
| | yl)-3-methoxyisoxazole-5- | | |
| | carboxamide | | |
| | P2 | | |
| 651 | 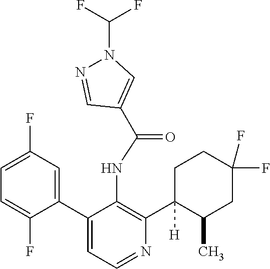 [SMILES: C[C@@H]1CC(F)(F)CC[C@@H]1c1nccc(-c2cc(F)ccc2F)c1NC(=O)c1cnn(C(F)F)c1] | Chiralpak AD column (250 mm × 30 mm, 10 μm) eluting with 2-propanol containing 0.1% ammonium hydroxide in supercritical carbon dioxide | 0.821 min, 99%; (400 MHz, CDCl₃) δ 8.65 (d, J = 4.8 Hz, 1H), 8.21 (s, 1H), 7.86 (s, 1H), 7.34-7.03 (m, 6H), 3.41-3.27 (m, 1H), 2.72-2.41 (m, 2H), 2.37-2.25 (m, 1H), 2.21-2.10 (m, 1H), 2.04-1.82 (m, 3H), 0.85 (d, J = 7.2 Hz, 3H); 483.1 (M + 1) |
| | N-(2-(syn-4,4-difluoro-2- | | |
| | methylcyclohexyl)-4-(2,5- | | |
| | difluorophenyl)pyridin-3- | | |
| | yl)-1-(difluoromethyl)-1H- | | |
| | pyrazole-4-carboxamide | | |
| | P1 | | |
| 652 | 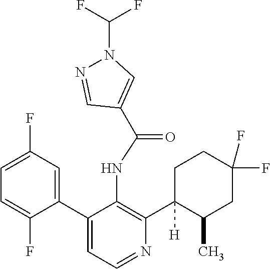 [SMILES: C[C@@H]1CC(F)(F)CC[C@@H]1c1nccc(-c2cc(F)ccc2F)c1NC(=O)c1cnn(C(F)F)c1] | Chiralpak AD column (250 mm × 30 mm, 10 μm) eluting with 2-propanol containing 0.1% ammonium hydroxide in supercritical carbon dioxide | 0.942 min, 99%; (400 MHz, CDCl₃) δ 8.65 (d, J = 4.8 Hz, 1H), 8.21 (s, 1H), 7.86 (s, 1H), 7.34-7.04 (m, 6H), 3.41-3.29 (m, 1H), 2.73-2.40 (m, 2H), 2.37-2.25 (m, 1H), 2.21-2.09 (m, 1H), 2.06-1.80 (m, 3H), 0.85 (d, J = 7.2 Hz, 3H); 483.1 (M + 1) |
| | N-(2-(syn-4,4-difluoro-2- | | |
| | methylcyclohexyl)-4-(2,5- | | |
| | difluorophenyl)pyridin-3- | | |
| | yl)-1-(difluoromethyl)-1H- | | |
| | pyrazole-4-carboxamide | | |
| | P2 | | |
| 653 | 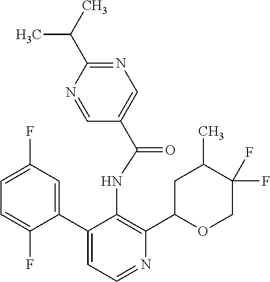 [SMILES: CC(C)c1ncc(C(=O)Nc2c(-c3cc(F)ccc3F)ccnc2C2CC(C)C(F)(F)CO2)cn1] | Chiralpak AD column (250 mm × 30 mm, 10 μm) eluting with 2-propanol containing 0.1% ammonium hydroxide in supercritical carbon dioxide | 1.182 min, 99%; (400 MHz, DMSO-d₆) δ 10.35 (s, 1H), 8.95 (s, 2H), 8.66 (d, J = 4.8 Hz, 1H), 7.54 (d, J = 4.8 Hz, 1H), 7.40-7.33 (m, 1H), 7.31-7.27 (m, 2H), 5.20-5.18 (m, 1H), 385-3.79 (m, 1H), 3.66-3.63 (m, 1H), 3.16 (m, 1H), 2.61- 2.58 (m, 1H), 2.45-2.41 (m, 1H), 1.84-1.82 (m, 1H), 1.28 (d, J = 6.8 Hz, 6H), 1.28 (d, J = 7.2 Hz, 3H); 489.2 (M + 1) |
| | N-(2-(5,5-difluoro-4- | | |
| | methyltetrahydro-2H- | | |
| | pyran-2-yl)-4-(2,5- | | |
| | difluorophenyl)pyridin-3- | | |
| | yl)-2-isopropylpyrimidine- | | |
| | 5-carboxamide | | |
| | P1, D1 | | |
| 654 | 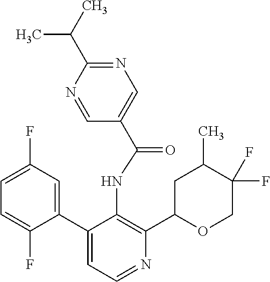 [SMILES: CC(C)c1ncc(C(=O)Nc2c(-c3cc(F)ccc3F)ccnc2C2CC(C)C(F)(F)CO2)cn1] | Chiralpak AD column (250 mm × 30 mm, 10 μm) eluting with 2-propanol containing 0.1% ammonium hydroxide in supercritical carbon dioxide | 1.396 min, 99%; (400 MHz, DMSO-d₆) δ 10.34 (s, 1H), 8.95 (s, 2H), 8.67 (d, J = 4.8 Hz, 1H), 7.54 (d, J = 4.8 Hz, 1H), 7.33-7.31 (m, 1H), 7.30-7.27 (m, 2H), 5.20 (d, J = 4.0 Hz, 1H), 3.86-3.68 (m, 1H), 3.66- 3.62 (m, 1H), 3.23-3.16 (m, 1H), 2.65-2.59 (m, 1H), 2.45- 2.42 (m, 1H), 1.87-1.80 (m, 1H), 1.28 (d, J = 6.8 Hz, 6H), 1.12 (d, J = 7.2 Hz, 3H); 489.2 (M + 1) |
| | N-(2-(5,5-difluoro-4- | | |
| | methyltetrahydro-2H- | | |
| | pyran-2-yl)-4-(2,5- | | |
| | difluorophenyl)pyridin-3- | | |
| | yl)-2-isopropylpyrimidine- | | |
| | 5-carboxamide | | |
| | P2, D1 | | |
| 655 | 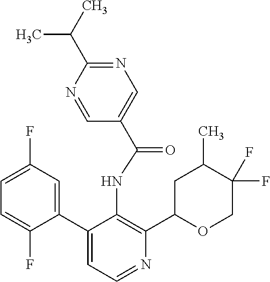 [SMILES: CC(C)c1ncc(C(=O)Nc2c(-c3cc(F)ccc3F)ccnc2C2CC(C)C(F)(F)CO2)cn1] | Chiralpak IC column (250 mm × 30 mm, 10 μm) eluting with 2-propanol containing 0.1% ammonium hydroxide in supercritical carbon dioxide | 1.135 min, 99%; (400 MHz, DMSO-d₆) δ 10.34 (s, 1H), 8.95 (s, 2H), 8.67 (d, J = 4.8 Hz, 1H), 7.54 (d, J = 4.8 Hz, 1H), 7.33-7.31 (m, 1H), 7.30-7.27 (m, 2H), 5.20 (d, J = 4.0 Hz, 1H), 3.86-3.68 (m, 1H), 3.66- 3.62 (m, 1H), 3.23-3.16 (m, 1H), 2.65-2.59 (m, 1H), 2.45- 2.42 (m, 1H), 1.87-1.80 (m, 1H), 1.28 (d, J = 6.8 Hz, 6H), 1.12 (d, J = 7.2 Hz, 3H); 489.2 (M + 1) |
| | N-(2-(5,5-difluoro-4- | | |
| | methyltetrahydro-2H- | | |
| | pyran-2-yl)-4-(2,5- | | |
| | difluorophenyl)pyridin-3- | | |
| | yl)-2-isopropylpyrimidine- | | |
| | 5-carboxamide | | |
| | P1, D2 | | |
| 656 | 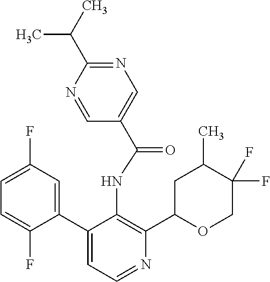 [SMILES: CC(C)c1ncc(C(=O)Nc2c(-c3cc(F)ccc3F)ccnc2C2CC(C)C(F)(F)CO2)cn1] | Chiralpak AD column (250 mm × 30 mm, 10 μm) eluting with 2-propanol containing 0.1% ammonium hydroxide in supercritical carbon dioxide | 1.396 min, 99% ee; (400 MHz, DMSO-d₆) δ 10.34 (s, 1H), 8.95 (s, 2H), 8.67 (d, J = 4.8 Hz, 1H), 7.54 (d, J = 4.8 Hz, 1H), 7.33-7.31 (m, 1H), 7.30-7.27 (m, 2H), 5.20 (d, J = 4.0 Hz, 1H), 3.86-3.68 (m, 1H), 3.66- 3.62 (m, 1H), 3.23-3.16 (m, 1H), 2.65-2.59 (m, 1H), 2.45- 2.42 (m, 1H), 1.87-1.80 (m, 1H), 1.28 (d, J = 6.8 Hz, 6H), 1.12 (d, J = 7.2 Hz, 3H); 489.2 (M + 1) |
| | N-(2-(5,5-difluoro-4- | | |
| | methyltetrahydro-2H- | | |
| | pyran-2-yl)-4-(2,5- | | |
| | difluorophenyl)pyridin-3- | | |
| | yl)-2-isopropylpyrimidine- | | |
| | 5-carboxamide | | |
| | P2, D2 | | |
| 657 | 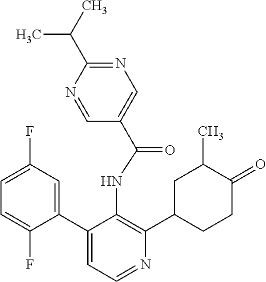 [SMILES: CC1CC(c2nccc(-c3cc(F)ccc3F)c2NC(=O)c2cnc(C(C)C)nc2)CCC1=O] | Chiralpak AD-H column (250 mm × 30 mm, 10 μm) eluting with 25% ethanol containing 0.1% ammonium hydroxide in supercritical carbon dioxide | 1.532 min, 97%; (400 MHz, CDCl₃) δ 8.91 (s, 2H), 8.60 (d, J = 5.2 Hz, 1H), 7.54 ( s, 1H), 7.15 (d, J = 5.2 Hz, 1H), 7.13- 6.98 (m, 3H), 3.46-3.36 (m, 1H), 3.28-3.16 (m, 1H), 2.50- 2.30 (m, 3H), 2.23-2.11 (m, 3H), 2.06-1.91 (m, 1H), 1.30 (d, J = 6.8 Hz, 6H), 1.00 (d, J = 6.4 Hz, 3H); 465.3 (M + 1) |
| | N-(4-(2,5-difluorophenyl)- | | |
| | 2-(3-methyl-4- | | |
| | oxocyclohexyl)pyridin-3- | | |
| | yl)-2-isopropylpyrimidine- | | |
| | 5-carboxamide | | |
| | P1, D2 | | |
| 658 | 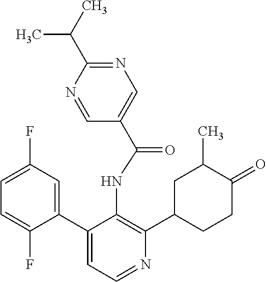 [SMILES: CC1CC(c2nccc(-c3cc(F)ccc3F)c2NC(=O)c2cnc(C(C)C)nc2)CCC1=O] | Chiralpak AD-H column (250 mm × 30 mm, 10 μm) eluting with 25% ethanol containing 0.1% ammonium hydroxide in supercritical carbon dioxide | 1.388 min, 93%; (400 MHz, CDCl₃) δ 8.91 (s, 2H), 8.60 (d, J = 4.8 Hz, 1H), 7.54-7.49 (m, 1H), 7.15 (d, J = 4.8 Hz, 1H), 7.12-7.00 (m, 3H), 3.47-3.36 (m, 1H), 3.30-3.17 (m, 1H), 2.49-2.29 (m, 3H), 2.25-2.12 (m, 3H), 2.06-1.90 (m, 1H), 1.30 (d, J = 6.8 Hz, 6H), 1.00 (d, J = 6.4 Hz, 3H); 465.2 (M + 1) |
| | N-(4-(2,5-difluorophenyl)- | | |
| | 2-(3-methyl-4- | | |
| | oxocyclohexyl)pyridin-3- | | |
| | yl)-2-isopropylpyrimidine- | | |
| | 5-carboxamide | | |
| | P2, D2 | | |
| 659 | 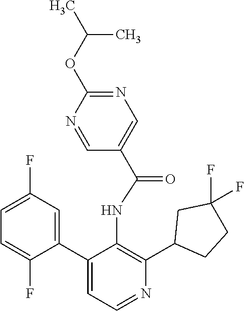 [SMILES: CC(C)Oc1ncc(C(=O)Nc2c(-c3cc(F)ccc3F)ccnc2C2CCC(F)(F)C2)cn1] | Chiralpak IC column (250 mm × 30 mm, 20 μm) eluting with 15% 2- propanol containing 0.1% ammonium hydroxide in supercritical carbon dioxide | 1.242 min, 99%; (400 MHz, DMSO-d₆) δ 10.21 (s, 1H), 8.86 (s, 2H), 8.65 (d, J = 4.8 Hz, 1H), 7.40 (d, J = 4.8 Hz, 1H), 7.37-7.30 (m, 1H), 7.30-7.18 (m, 2H), 5.26 (sept, J = 6.0 Hz, 1H), 3.80 (quin, J = 8.4 Hz, 1H), 2.58 (s, 1H), 2.46-2.35 (m, 1H), 2.34-2.22 (m, 1H), 2.21-2.03 (m, 2H), 2.02-1.90 (m, 1H), 1.33 (d, J = 6.0 Hz, 6H); 475.2 (M + 1) |
| | N-(2-(3,3- | | |
| | difluorocyclopentyl)-4-(2,5- | | |
| | difluorophenyl)pyridin-3- | | |
| | yl)-2- | | |
| | isopropoxypyrimidine-5- | | |
| | carboxamide | | |
| | P1 | | |
| 660 | 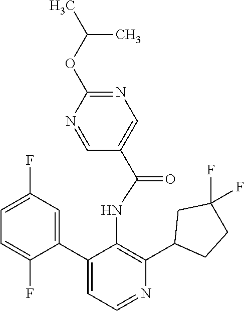 [SMILES: CC(C)Oc1ncc(C(=O)Nc2c(-c3cc(F)ccc3F)ccnc2C2CCC(F)(F)C2)cn1] | Chiralpak IC column (250 mm × 30 mm, 20 μm) eluting with 15% 2- propanol containing 0.1% ammonium hydroxide in supercritical carbon dioxide | 1.468 min, 97%; (400 MHz, DMSO-d₆) δ 10.21 (s, 1H), 8.86 (s, 2H), 8.65 (d, J = 4.8 Hz, 1H), 7.40 (d, J = 4.8 Hz, 1H), 7.37-7.30 (m, 1H), 7.30-7.18 (m, 2H), 5.26 (quin, J = 6.0 Hz, 1H), 3.86-3.75 (m, 1H), 2.58- 2.52 (m, 1H), 2.45-2.36 (m, 1H), 2.33-2.23 (m, 1H), 2.20- 2.03 (m, 2H), 2.02-1.87 (m, 1H), 1.33 (d, J = 6.0 Hz, 6H); 475.2 (M + 1) |
| | N-(2-(3,3- | | |
| | difluorocyclopentyl)-4-(2,5- | | |
| | difluorophenyl)pyridin-3- | | |
| | yl)-2- | | |
| | isopropoxypyrimidine-5- | | |
| | carboxamide | | |
| | P2 | | |
| 661 | 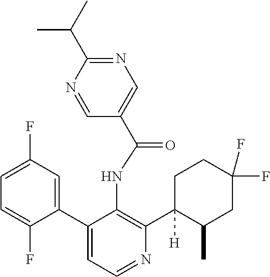 [SMILES: CC(C)c1ncc(C(=O)Nc2c(-c3cc(F)ccc3F)ccnc2[C@H]2CCC(F)(F)C[C@H]2C)cn1] | Chiralcel OJ column (250 mm × 30 mm, 10 μm) eluting with 20% methanol containing 0.1% ammonium hydroxide in supercritical carbon dioxide | 0.853 min, 99%; (400 MHz, CD₃OD) δ 8.93 (s, 2H), 8.64 (d, J = 4.8 Hz, 1H), 7.35 (d, J = 4.8 Hz, 1H), 7.25-7.18 (m, 1H), 7.17-7.10 (m, 2H), 3.51-3.41 (m, 1H) 3.28-3.20 (m, 1H), 2.70-2.47 (m, 1H), 2.44-2.26 (m, 2H), 2.23-2.11 (m, 1H), 2.02-1.79 (m, 3H), 1.34 (d, J = 6.8 Hz, 6H), 0.85 (d, J = 7.2 Hz, 3H); 487.2 (M + 1) |
| | N-(2-(syn-4,4-difluoro-2- | | |
| | methylcyclohexyl)-4-(2,5- | | |
| | difluorophenyl)pyridin-3- | | |
| | yl)-2-isopropylpyrimidine- | | |
| | 5-carboxamide | | |
| | P1 | | |
| 662 | 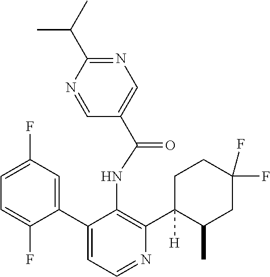 [SMILES: CC(C)c1ncc(C(=O)Nc2c(-c3cc(F)ccc3F)ccnc2[C@H]2CCC(F)(F)C[C@H]2C)cn1] | Chiralcel OJ column (250 mm × 30 mm, 10 μm) eluting with 20% methanol containing 0.1% ammonium hydroxide in supercritical carbon dioxide | 1.205 min, 99%; (400 MHz, CD₃OD) δ 8.93 (s, 2H), 8.64 (d, J = 4.8 Hz, 1H), 7.35 (d, J = 4.8 Hz, 1H), 7.25-7.18 (m, 1H), 7.17-7.10 (m, 2H), 3.51-3.41 (m, 1H) 3.28-3.20 (m, 1H), 2.70-2.47 (m, 1H), 2.44-2.26 (m, 2H), 2.23-2.11 (m, 1H), 2.02-1.79 (m, 3H), 1.34 (d, J = 6.8 Hz, 6H), 0.85 (d, J = 7.2 Hz, 3H); 487.2 (M + 1) |
| | N-(2-(syn-4,4-difluoro-2- | | |
| | methylcyclohexyl)-4-(2,5- | | |
| | difluorophenyl)pyridin-3- | | |
| | yl)-2-isopropylpyrimidine- | | |
| | 5-carboxamide | | |
| | P2 | | |
| 663 | 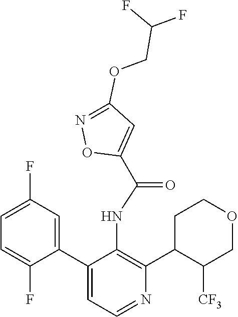 [SMILES: O=C(Nc1c(-c2cc(F)ccc2F)ccnc1C1CCOCC1C(F)(F)F)c1cc(OCC(F)F)no1] | ChiralPak IG column (250 mm × 10 mm) eluting with 5% methanol containing 10 mM ammonium formate in supercritical carbon dioxide | 2.40 min, 99%; (400 MHz, DMSO-d₆) δ 10.67 (s, 1H), 8.65 (d, J = 3.9 Hz, 1H), 7.42 (d, J = 4.6 Hz, 1H), 7.28 (ddtd, J = 32.5, 15.6, 7.9, 3.9 Hz, 3H), 6.95 (s, 1H), 6.41 (tt, J = 53.9, 3.1 Hz, 1H), 4.56 (td, J = 14.9, 3.1 Hz, 2H), 4.22 (dd, J = 12.3, 2.1 Hz, 1H), 4.11-4.07 (m, 1H), 3.75 (s, 1H), 3.64 (d, J = 11.9 Hz, 1H), 3.58-3.52 (m, 1H), 2.87 (s, 1H), 2.67-2.53 (m, 1H), 1.58 (d, J = 13.5 Hz, 1H); 534.2 (M + 1) |
| | 3-(2,2-difluoroethoxy)-N- | | |
| | (4-(2,5-difluorophenyl)-2- | | |
| | (3- | | |
| | (trifluoromethyl)tetrahydro- | | |
| | 2H-pyran-4-yl)pyridin-3- | | |
| | yl)isoxazole-5-carboxamide | | |
| | P1 | | |
| 664 | 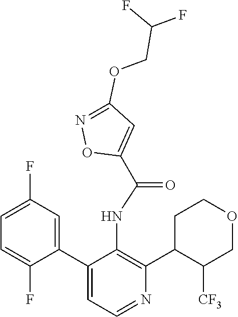 [SMILES: O=C(Nc1c(-c2cc(F)ccc2F)ccnc1C1CCOCC1C(F)(F)F)c1cc(OCC(F)F)no1] | ChiralPak IG column (250 mm × 10 mm) eluting with 5% methanol containing 10 mM ammonium formate in supercritical carbon dioxide | 2.62 min, 99%; (400 MHz, DMSO-d₆) δ 10.66 (s, 1H), 8.65 (d, J = 4.5 Hz, 1H), 7.42 (d, J = 4.9 Hz, 1H), 7.37-7.20 (m, 3H), 6.95 (s, 1H), 6.41 (tt, J = 53.8, 3.1 Hz, 1H), 4.56 (td, J = 15.0, 3.1 Hz, 2H), 4.22 (dd, J = 12.4, 2.1 Hz, 1H), 4.09 (dt, J = 11.0, 3.3 Hz, 1H), 3.74 (s, 1H), 3.64 (d, J = 11.9 Hz, 1H), 3.55 (td, J = 11.2, 2.1 Hz, 1H), 2.86 (s, 1H), 2.68-2.53 (m, 1H), 1.59 (d, J = 11.6 Hz, 1H); 534.2 (M + 1) |
| | 3-(2,2-difluoroethoxy)-N- | | |
| | (4-(2,5-difluorophenyl)-2- | | |
| | (3- | | |
| | (trifluoromethyl)tetrahydro- | | |
| | 2H-pyran-4-yl)pyridin-3- | | |
| | yl)isoxazole-5-carboxamide | | |
| | P2 | | |
| 665 | 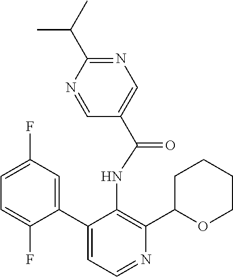 [SMILES: CC(C)c1ncc(C(=O)Nc2c(-c3cc(F)ccc3F)ccnc2C2CCCCO2)cn1] | ChiralPak IC column (250 mm × 10 mm) eluting with 30% 1:1 acetoni- trile:ethanol containing 10 mM ammonium formate in supercritical carbon dioxide | 3.36 min, 99%; (400 MHz, DMSO-d₆) δ 10.36 (s, 1H), 8.98 (s, 2H), 8.64 (d, J = 4.9 Hz, 1H), 7.47 (d, J = 4.8 Hz, 1H), 7.35 (td, J = 9.6, 4.6 Hz, 1H), 7.27 (td, J = 7.1, 3.2 Hz, 2H), 4.68-4.66 (m, 1H), 3.95-3.92 (m, 1H), 3.51-3.45 (m, 1H), 3.19 (sept, J = 6.9 Hz, 1H), 1.98-1.86 (m, 2H), 1.71 (d, J = 13.3 Hz, 1H), 1.60-1.48 (m, 3H), 1.28 (d, J = 6.9 Hz, 6H); 439.2 (M + 1) |
| | N-(4-(2,5-difluorophenyl)- | | |
| | 2-(tetrahydro-2H-pyran-2- | | |
| | yl)pyridin-3-yl)-2- | | |
| | isopropylpyrimidine-5- | | |
| | carboxamide | | |
| | P1 | | |
| 666 | 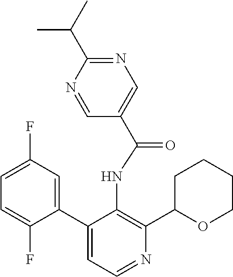 [SMILES: CC(C)c1ncc(C(=O)Nc2c(-c3cc(F)ccc3F)ccnc2C2CCCCO2)cn1] | ChiralPak IC column (250 mm × 10 mm) eluting with 30% 1:1 acetoni- trile:ethanol containing 10 mM ammonium formate in supercritical carbon dioxide | 3.97 min, 99%; (400 MHz, DMSO-d₆) δ 10.36 (s, 1H), 8.98 (s, 2H), 8.64 (d, J = 4.9 Hz, 1H), 7.47 (d, J = 4.8 Hz, 1H), 7.35 (td, J = 9.6, 4.6 Hz, 1H), 7.27 (td, J = 7.1, 3.2 Hz, 2H), 4.68-4.66 (m, 1H), 3.95-3.92 (m, 1H), 3.51-3.45 (m, 1H), 3.19 (sept, J = 6.9 Hz, 1H), 1.98-1.86 (m, 2H), 1.71 (d, J = 13.3 Hz, 1H), 1.60-1.48 (m, 3H), 1.28 (d, J = 6.9 Hz, 6H); 439.2 (M + 1) |
| | N-(4-(2,5-difluorophenyl)- | | |
| | 2-(tetrahydro-2H-pyran-2- | | |
| | yl)pyridin-3-yl)-2- | | |
| | isopropylpyrimidine-5- | | |
| | carboxamide | | |
| | P2 | | |

### Biological Example 1

As disclosed above, typical assays for testing compounds of the disclosure are known, for example as disclosed in Crestey, F. et al., ACS Chem Neurosci (2015), Vol. 6, pp. 1302-1308, AA43279 (Frederiksen, K. et al., Eur J Neurosci (2017), Vol. 46, pp. 1887-1896) and Lu AE98134 (von Schoubyea, N. L. et al., Neurosci Lett (2018), Vol. 662, pp. 29-35) which employs the use of automated planar patch clamp techniques to study the effects of the chemical agent on the gating of sodium channels. The sodium channel isoforms of interest are stably expressed in Human Embryonic Kidney Cells and the currents that flow through those channels in response to a depolarizing voltage clamp step from −120 mV to 0 mV are measured in the presence of increasing concentrations of the chemical agents. The area under the sodium current trace which correlates to the magnitude of sodium flux through the cell mebrane is used to quantify the effects on gating of the channels. Other parameters that are measured in the assay include the peak current, time constant of open state inactivation and the voltage dependence of steady state inactivation properties. The concentration responses are used to determine potency of each chemical agents effects on modulating the sodium channel isoform gating.

Each of the aforementioned references are hereby incorporated by reference in their entirety.

TABLE 2
Biological activity of representative
compounds of formula (I) or (II)

| | | Empirical |
|---|---|---|
| Example No. | EC₅₀ | Eₘₐₓ |
| 1 | ++++ | ++++ |
| 2 | ++++ | ++++ |
| 3 | ++++ | ++++ |
| 4 | ++++ | ++++ |
| 5 | +++ | +++ |
| 6 | ++++ | +++ |
| 7 | ++++ | ++++ |
| 8 | ++++ | +++ |
| 9 | ++++ | +++ |
| 10 | ++ | ++ |
| 11 | ++++ | + |
| 12 | ++++ | + |
| 13 | ++++ | + |
| 14 | ++ | +++ |
| 15 | ++++ | ++++ |
| 16 | ++ | ++ |
| 17 | +++ | +++ |
| 18 | ++ | +++ |
| 19 | ++ | ++ |
| 20 | + | + |
| 21 | + | + |
| 22 | +++ | +++ |
| 23 | +++ | ++++ |
| 24 | +++ | ++++ |
| 25 | ++++ | ++++ |
| 26 | +++ | +++ |
| 27 | ++++ | +++ |
| 28 | +++ | ++ |
| 29 | +++ | + |
| 30 | +++ | ++ |
| 31 | ++++ | + |
| 32 | +++ | +++ |
| 33 | +++ | +++ |
| 34 | ++++ | +++ |
| 35 | +++ | +++ |
| 36 | ++++ | +++ |
| 37 | ++++ | +++ |
| 38 | + | + |
| 39 | + | + |
| 40 | ++ | + |
| 41 | ++ | ++ |
| 42 | ++++ | + |
| 43 | + | ++ |
| 44 | ++++ | + |
| 45 | ++++ | + |
| 46 | +++ | +++ |
| 47 | ++++ | +++ |
| 48 | +++ | +++ |
| 49 | ++++ | + |
| 50 | + | + |
| 51 | +++ | + |
| 52 | ++ | + |
| 53 | +++ | ++ |
| 54 | ++++ | ++++ |
| 55 | +++ | +++ |
| 56 | ++++ | — |
| 57 | ++++ | — |
| 58 | ++++ | — |
| 59 | ++++ | — |
| 60 | ++++ | — |
| 61 | ++++ | — |
| 62 | +++ | — |
| 63 | — | — |
| 64 | — | — |
| 65 | — | — |
| 66 | — | — |
| 67 | — | — |
| 68 | — | — |
| 69 | — | — |
| 70 | — | — |
| 71 | — | — |
| 72 | — | — |
| 73 | — | — |
| 74 | — | — |
| 75 | — | — |
| 76 | — | — |
| 77 | — | — |
| 78 | ++++ | — |
| 79 | + | — |
| 80 | ++++ | — |
| 81 | ++++ | — |
| 82 | ++++ | — |
| 83 | +++ | — |
| 84 | ++ | — |
| 85 | ++++ | — |
| 86 | +++ | — |
| 87 | +++ | — |
| 88 | +++ | — |
| 89 | ++ | — |
| 90 | ++++ | — |
| 91 | ++++ | — |
| 92 | ++++ | — |
| 93 | ++++ | — |
| 94 | ++++ | — |
| 95 | ++ | — |
| 96 | +++ | — |
| 97 | ++++ | — |
| 98 | ++++ | — |
| 99 | ++++ | — |
| 100 | ++ | — |
| 101 | ++++ | — |
| 102 | ++++ | — |
| 103 | ++++ | — |
| 104 | ++++ | — |
| 105 | ++++ | — |
| 106 | ++++ | — |
| 107 | +++ | — |
| 108 | ++++ | — |
| 109 | ++++ | — |
| 110 | ++++ | — |
| 111 | +++ | — |
| 112 | ++++ | — |
| 113 | +++ | — |
| 114 | ++++ | — |
| 115 | +++ | — |
| 116 | +++ | — |
| 117 | +++ | — |
| 118 | ++++ | — |
| 119 | ++++ | — |
| 120 | +++ | — |
| 121 | ++++ | — |
| 122 | ++++ | — |
| 123 | ++++ | — |
| 124 | ++++ | — |
| 125 | — | — |
| 126 | ++++ | — |
| P1 | | |
| 127 | ++++ | — |
| P2 | | |
| 128 | ++++ | — |
| 129 | ++++ | — |
| 130 | ++++ | — |
| 131 | ++++ | — |
| 132 | ++++ | — |
| 133 | ++++ | — |
| 134 | ++++ | — |
| 135 | ++++ | — |
| 136 | ++++ | — |
| 137 | ++++ | — |
| 138 | ++++ | — |
| 139 | ++++ | — |
| 140 | ++++ | — |
| 141 | ++++ | — |
| 142 | ++++ | — |
| 143 | ++++ | — |
| 144 | ++++ | — |
| 145 | ++++ | — |
| 146 | ++++ | — |
| 147 | ++++ | — |
| 148 | ++++ | — |
| 149 | +++ | — |
| 150 | +++ | — |
| 151 | ++++ | — |
| 152 | — | — |
| 153 | ++++ | — |
| 154 | ++++ | — |
| 155 | ++++ | — |
| 156 | ++++ | — |
| 157 | ++++ | — |
| 158 | ++++ | — |
| 159 | ++++ | — |
| 160 | ++++ | — |
| 161 | ++++ | — |
| 162 | ++++ | — |
| 163 | ++++ | — |
| 164 | ++++ | — |
| 165 | ++++ | — |
| 166 | ++++ | — |
| 167 | ++++ | — |
| 168 | ++++ | — |
| 169 | ++++ | — |
| 170 | ++++ | — |
| 171 | +++ | — |
| 172 | +++ | — |
| 173 | ++++ | — |
| 174 | ++++ | — |
| 175 | ++++ | — |
| 176 | ++++ | — |
| 177 | ++++ | — |
| 178 | ++++ | — |
| 179 | ++++ | — |
| 180 | ++++ | — |
| 181 | — | — |
| 182 | ++++ | — |
| 183 | ++++ | — |
| 184 | ++++ | — |
| 185 | ++++ | — |
| 186 | ++ | — |
| 187 | ++ | — |
| 188 | ++ | — |
| 189 | — | — |
| 190 | — | — |
| 191 | — | — |
| 192 | — | — |
| 193 | — | — |
| 194 | — | — |
| 195 | ++++ | — |
| 196 | ++++ | — |
| 197 | ++++ | — |
| 198 | ++++ | — |
| 199 | +++ | — |
| 200 | ++++ | — |
| 201 | +++ | — |
| 202 | ++++ | — |
| 203 | ++ | — |
| 204 | — | — |
| 205 | — | — |
| 206 | — | — |
| 207 | — | — |
| 208 | — | — |
| 209 | — | — |
| 210 | ++++ | — |
| 211 | — | — |
| 212 | ++++ | — |
| P1 | | |
| 213 | ++ | — |
| P2 | | |
| 214 | — | — |
| P1 | | |
| 215 | — | — |
| P2 | | |
| 216 | — | — |
| P1 | | |
| 217 | — | — |
| P2 | | |
| 218 | ++ | — |
| P1 | | |
| 219 | +++ | — |
| P2 | | |
| 220 | — | — |
| 221 | ++ | — |
| 222 | ++ | — |
| 223 | +++ | — |
| 224 | +++ | — |
| 225 | ++++ | — |
| 226 | ++++ | — |
| P1 | | |
| 227 | ++ | — |
| P2 | | |
| 228 | ++++ | — |
| 229 | ++ | — |
| 230 | +++ | — |
| 231 | +++ | — |
| 232 | +++ | — |
| 233 | +++ | — |
| 234 | +++ | — |
| 235 | ++ | — |
| P1 | | |
| 236 | ++ | — |
| P2 | | |
| 237 | ++++ | — |
| 238 | — | — |
| 239 | — | — |
| 240 | — | — |
| 241 | ++++ | — |
| 242 | ++++ | — |
| 243 | ++++ | — |
| 244 | ++++ | — |
| 245 | ++++ | — |
| 246 | ++++ | — |
| 247 | +++ | — |
| 248 | ++++ | — |
| 249 | +++ | — |
| 250 | +++ | — |
| 251 | ++++ | — |
| 252 | ++++ | — |
| 253 | +++ | — |
| 254 | ++ | — |
| 255 | ++++ | — |
| 256 | ++++ | — |
| 257 | ++++ | — |
| 258 | ++++ | — |
| 259 | ++++ | — |
| 260 | ++++ | — |
| 261 | ++++ | — |
| 262 | ++++ | — |
| 263 | ++++ | — |
| 264 | +++ | — |
| 265 | ++++ | — |
| 266 | ++++ | — |
| 267 | ++++ | — |
| 268 | +++ | — |
| 269 | ++ | — |
| 270 | +++ | — |
| 271 | +++ | — |
| 272 | ++++ | — |
| 273 | ++ | — |
| 274 | +++ | — |
| 275 | +++ | — |
| 276 | ++ | — |
| 277 | +++ | — |
| 278 | ++++ | — |
| 279 | ++ | — |
| 280 | ++ | — |
| 281 | ++++ | — |
| 282 | ++++ | — |
| 283 | +++ | — |
| 284 | +++ | — |
| 285 | ++++ | — |
| 286 | ++++ | — |
| 287 | ++++ | — |
| 288 | — | — |
| 289 | +++ | — |
| 290 | +++ | — |
| 291 | ++++ | — |
| 292 | ++++ | — |
| 293 | ++++ | — |
| 294 | ++++ | — |
| 295 | ++++ | — |
| 296 | ++++ | — |
| P1 | | |
| 297 | ++++ | — |
| P2 | | |
| 298 | ++++ | — |
| 299 | ++++ | — |
| 300 | ++++ | — |
| P1 | | |
| 301 | ++++ | — |
| P2 | | |
| 302 | ++++ | — |
| 303 | ++++ | — |
| 304 | ++++ | — |
| 305 | ++++ | — |
| 306 | ++++ | — |
| 307 | ++++ | — |
| 308 | ++++ | — |
| 309 | ++++ | — |
| 310 | ++ | — |
| 311 | ++++ | — |
| 312 | ++ | — |
| 313 | ++++ | — |
| 314 | ++++ | — |
| 315 | ++ | — |
| 316 | ++++ | — |
| 317 | ++++ | — |
| 318 | ++++ | — |
| 319 | ++++ | — |
| 320 | — | — |
| 321 | ++++ | — |
| 322 | ++++ | — |
| 323 | ++++ | — |
| 324 | ++++ | — |
| 325 | +++ | — |
| 326 | ++++ | — |
| 327 | +++ | — |
| 328 | +++ | — |
| 329 | ++++ | — |
| 330 | +++ | — |
| 331 | ++++ | — |
| 332 | +++ | — |
| 333 | ++ | — |
| 334 | ++++ | — |
| 335 | — | — |
| P1 | | |
| 336 | — | — |
| P2 | | |
| 337 | — | — |
| P3 | | |
| 338 | — | — |
| P4 | | |
| 339 | — | — |
| P1 | | |
| 340 | — | — |
| P2 | | |
| 341 | — | — |
| P3 | | |
| 342 | — | — |
| P4 | | |
| 343 | ++++ | — |
| P1 | | |
| 344 | — | — |
| P1 | | |
| 345 | — | — |
| P2 | | |
| 346 | — | — |
| P1 | | |
| 347 | — | — |
| P2 | | |
| 348 | — | — |
| 349 | — | — |
| P1 | | |
| 350 | — | — |
| P2 | | |
| 351 | — | — |
| 352 | — | — |
| 353 | +++ | — |
| P1 | | |
| 354 | ++++ | — |
| P2 | | |
| 355 | +++ | — |
| P1 | | |
| 356 | ++++ | — |
| P2 | | |
| 357 | ++ | — |
| P1 | | |
| 358 | ++++ | — |
| P2 | | |
| 359 | +++ | — |
| P1 | | |
| 360 | ++++ | — |
| P2 | | |
| 361 | +++ | — |
| P1 | | |
| 362 | ++++ | — |
| P2 | | |
| 363 | +++ | — |
| P1 | | |
| 364 | ++++ | — |
| P2 | | |
| 365 | ++++ | — |
| P1 | | |
| 366 | +++ | — |
| P2 | | |
| 367 | ++++ | — |
| P1 | | |
| 368 | +++ | — |
| P2 | | |
| 369 | ++ | — |
| P1 | | |
| 370 | ++++ | — |
| P2 | | |
| 371 | ++ | — |
| P1 | | |
| 372 | ++++ | — |
| P2 | | |
| 373 | ++++ | — |
| P1 | | |
| 374 | ++ | — |
| P2 | | |
| 375 | ++++ | — |
| P1 | | |
| 376 | ++ | — |
| P2 | | |
| 377 | +++ | — |
| 378 | ++++ | — |
| 379 | ++++ | — |
| 380 | ++ | — |
| 381 | ++ | — |
| 382 | ++ | — |
| 383 | ++++ | — |
| 384 | +++ | — |
| 385 | ++ | — |
| 386 | ++ | — |
| 387 | — | — |
| 388 | — | — |
| 389 | — | — |
| 390 | +++ | — |
| 391 | ++ | — |
| 392 | ++++ | — |
| 393 | ++++ | — |
| 394 | ++ | — |
| 395 | ++++ | — |
| 396 | — | — |
| 397 | ++++ | — |
| 398 | ++++ | — |
| 399 | ++++ | — |
| 400 | ++ | — |
| 401 | +++ | — |
| 402 | +++ | — |
| 403 | ++++ | — |
| 404 | +++ | — |
| 405 | ++++ | — |
| D1 | | |
| 406 | ++++ | — |
| 407 | ++++ | — |
| 408 | +++ | — |
| 409 | +++ | — |
| P1 | | |
| 410 | ++++ | — |
| P2 | | |
| 411 | — | — |
| 412 | ++++ | — |
| 413 | ++++ | — |
| 414 | ++++ | — |
| 415 | ++++ | — |
| 416 | — | — |
| 417 | ++++ | — |
| 418 | ++++ | — |
| 419 | ++++ | — |
| 420 | +++ | — |
| 421 | +++ | — |
| 422 | +++ | — |
| 423 | +++ | — |
| 424 | ++ | — |
| 425 | +++ | — |
| 426 | ++ | — |
| 427 | ++ | — |
| 428 | ++++ | — |
| 429 | +++ | — |
| 430 | ++ | — |
| 431 | +++ | — |
| 432 | +++ | — |
| 433 | +++ | — |
| 434 | +++ | — |
| 435 | ++++ | — |
| 436 | +++ | — |
| 437 | ++++ | — |
| 438 | ++++ | — |
| 439 | — | — |
| 440 | ++++ | — |
| 441 | ++++ | — |
| 442 | ++++ | — |
| 443 | ++++ | — |
| 444 | ++++ | — |
| 445 | ++++ | — |
| 446 | ++++ | — |
| 447 | ++++ | — |
| P1 | | |
| 448 | ++++ | — |
| P2 | | |
| 449 | ++++ | — |
| 450 | ++++ | — |
| 451 | ++++ | — |
| 452 | — | — |
| 453 | ++++ | — |
| 454 | +++ | — |
| 455 | ++++ | — |
| 456 | ++++ | — |
| 457 | ++++ | — |
| 458 | — | — |
| 459 | — | — |
| 460 | — | — |
| 461 | — | — |
| 462 | — | — |
| 463 | — | — |
| 464 | ++++ | — |
| 465 | ++++ | — |
| 466 | ++++ | — |
| 467 | ++++ | — |
| 468 | +++ | — |
| P1 | | |
| 469 | ++++ | — |
| P2 | | |
| 470 | +++ | — |
| P1 | | |
| 471 | ++++ | — |
| P2 | | |
| 472 | ++++ | — |
| P1 | | |
| 473 | ++++ | — |
| P2 | | |
| 474 | +++ | — |
| P1 | | |
| 475 | ++++ | — |
| P2 | | |
| 476 | ++++ | — |
| P1 | | |
| 477 | +++ | — |
| P2 | | |
| 478 | +++ | — |
| P1 | | |
| 479 | ++++ | — |
| P2 | | |
| 480 | ++++ | — |
| P1 | | |
| 481 | ++++ | — |
| P2 | | |
| 482 | ++ | — |
| P1 | | |
| 483 | ++++ | — |
| P2 | | |
| 484 | +++ | — |
| P1 | | |
| 485 | ++++ | — |
| P2 | | |
| 486 | ++ | — |
| P1 | | |
| 487 | ++++ | — |
| P2 | | |
| 488 | +++ | — |
| P1 | | |
| 489 | ++++ | — |
| P2 | | |
| 490 | +++ | — |
| P1 | | |
| 491 | ++++ | — |
| P2 | | |
| 492 | +++ | — |
| P1 | | |
| 493 | ++++ | — |
| P2 | | |
| 494 | +++ | — |
| P1 | | |
| 495 | ++++ | — |
| P2 | | |
| 496 | +++ | — |
| P1 | | |
| 497 | ++++ | — |
| P2 | | |
| 498 | +++ | — |
| P1 | | |
| 499 | ++++ | — |
| P2 | | |
| 500 | — | — |
| P1 | | |
| 501 | — | — |
| P2 | | |
| 502 | — | — |
| 503 | +++ | — |
| P1 | | |
| 504 | ++++ | — |
| P2 | | |
| 505 | ++++ | — |
| 506 | — | — |
| 507 | — | — |
| 508 | ++++ | — |
| 509 | — | — |
| 510 | — | — |
| 511 | — | — |
| 512 | — | — |
| 513 | — | — |
| P1 | | |
| 514 | — | — |
| P2 | | |
| 515 | — | — |
| P1 | | |
| 516 | — | — |
| P2 | | |
| 517 | — | — |
| P1 | | |
| 518 | — | — |
| P2 | | |
| 519 | — | — |
| P1 | | |
| 520- | — | — |
| P2 | | |
| 521 | — | — |
| P1 | | |
| 522 | — | — |
| P2 | | |
| 523 | — | — |
| P1 | | |
| 524 | — | — |
| P2 | | |
| 525 | — | — |
| P1 | | |
| 526 | — | — |
| P2 | | |
| 527 | +++ | — |
| 528 | ++++ | — |
| P1 | | |
| 529 | ++++ | — |
| P2 | | |
| 530 | ++++ | — |
| 531 | ++++ | — |
| 532 | ++++ | — |
| P1 | | |
| 533 | — | — |
| P2 | | |
| 534 | ++++ | — |
| P1 | | |
| 535 | ++++ | — |
| P2 | | |
| 536 | +++ | — |
| 537 | +++ | — |
| 538 | ++++ | — |
| 539 | ++++ | — |
| P1, D1 | | |
| 540 | ++++ | — |
| P2, D1 | | |
| 541 | ++++ | — |
| P1 | | |
| 542 | ++++ | — |
| P2 | | |
| 543 | — | — |
| 544 | +++ | — |
| 545 | +++ | — |
| 546 | +++ | — |
| D1 | | |
| 547 | ++ | — |
| D2 | | |
| 548 | ++ | — |
| 549 | ++ | — |
| 550 | ++ | — |
| 551 | ++ | — |
| D1 | | |
| 552 | ++ | — |
| D2 | | |
| 553 | ++ | — |
| 554 | ++ | — |
| 555 | ++++ | — |
| 556 | ++ | — |
| 557 | ++++ | — |
| 558 | ++ | — |
| 559 | +++ | — |
| 560 | +++ | — |
| 561 | +++ | — |
| 562 | ++++ | — |
| 563 | ++++ | — |
| 564 | ++ | — |
| 565 | ++ | — |
| 566 | — | — |
| 567 | — | — |
| 568 | +++ | — |
| 569 | +++ | — |
| 570 | ++ | — |
| 571 | ++ | — |
| 572 | +++ | — |
| 573 | ++++ | — |
| 574 | ++ | — |
| 575 | — | — |
| 576 | ++++ | — |
| 577 | ++ | — |
| 578 | ++ | — |
| 579 | ++ | — |
| 580 | ++ | — |
| 581 | +++ | — |
| P1 | | |
| 582 | +++ | — |
| P2 | | |
| 583 | ++++ | — |
| 584 | ++++ | — |
| P1 | | |
| 585 | ++++ | — |
| P2 | | |
| 586 | ++++ | — |
| 587 | +++ | — |
| 588 | ++ | — |
| 589 | +++ | — |
| P1 | | |
| 590 | +++ | — |
| P2 | | |
| 591 | ++++ | — |
| 592 | ++++ | — |
| 593 | ++++ | — |
| 594 | ++ | — |
| D1 | | |
| 595 | ++ | — |
| D2 | | |
| 596 | ++ | — |
| 597 | ++++ | — |
| D1 | | |
| 598 | ++++ | — |
| D2 | | |
| 599 | ++ | — |
| D1 | | |
| 600 | ++++ | — |
| D2 | | |
| 601 | — | — |
| 602 | ++++ | — |
| 603 | ++ | — |
| D1 | | |
| 604 | ++ | — |
| D2 | | |
| 605 | +++ | — |
| 606 | +++ | — |
| 607 | ++++ | — |
| 608 | — | — |
| 609 | — | — |
| 610 | — | — |
| 611 | — | — |
| 612 | ++ | — |
| 613 | ++++ | — |
| 614 | — | — |
| 615 | ++++ | — |
| 616 | ++++ | — |
| 617 | ++++ | — |
| 618 | ++++ | — |
| 619 | ++++ | — |
| 620 | ++++ | — |
| 621 | ++++ | — |
| 622 | ++++ | — |
| 623 | ++++ | — |
| 624 | ++++ | — |
| 625 | — | — |
| 626 | ++++ | — |
| 627 | — | — |
| 628 | ++++ | — |
| 629 | ++++ | — |
| 630 | ++++ | — |
| 631 | ++++ | — |
| 632 | ++++ | — |
| 633 | — | — |
| 634 | — | — |
| 635 | — | — |
| 636 | — | — |
| 637 | ++ | — |
| 638 | — | — |
| 639 | +++ | — |
| P1 | | |
| 640 | ++++ | — |
| P2 | | |
| 641 | ++++ | — |
| P1 | | |
| 642 | ++++ | — |
| P2 | | |
| 643 | ++++ | — |
| P1 | | |
| 644 | +++ | — |
| P2 | | |
| 645 | ++++ | — |
| P1 | | |
| 646 | ++++ | — |
| P2 | | |
| 647 | ++++ | — |
| P1 | | |
| 648 | +++ | — |
| P2 | | |
| 649 | ++++ | — |
| P1 | | |
| 650 | +++ | — |
| P2 | | |
| 651 | ++++ | — |
| P1 | | |
| 652 | ++++ | — |
| P2 | | |
| 653 | ++ | — |
| P1, D1 | | |
| 654 | ++++ | — |
| P2, D1 | | |
| 655 | ++ | — |
| P1, D2 | | |
| 656 | ++++ | — |
| P2, D2 | | |
| 657 | ++++ | — |
| P1, D2 | | |
| 658 | ++++ | — |
| P2, D2 | | |
| 659 | ++++ | — |
| P1 | | |
| 660 | ++++ | — |
| P2 | | |
| 661 | ++++ | — |
| P1 | | |
| 662 | ++++ | — |
| P2 | | |
| 663 | — | — |
| P1 | | |
| 664 | — | — |
| P2 | | |
| 665 | — | — |
| P1 | | |
| 666 | — | — |
| P2 | | |
| 667 | ++++ | — |
| 668 | ++++ | — |
| 669 | ++++ | — |
| 670 | ++++ | — |
| 671 | ++++ | — |
| 672 | +++ | — |
| 673 | +++ | — |
| 674 | ++++ | — |
| 675 | ++++ | — |
| 676 | ++++ | — |
| 677 | +++ | — |
| 678 | +++ | — |
| 679 | ++ | — |

For EC₅₀ values:
++++ indicates a value less than 1 μM
+++ indicates a value from 1 up to 10 μM
++ indicates a value from 10 up to 50 μM
+ indicates a value of 50 μM or more
For Empirical Eₘₐₓ values:
++++ indicates a value greater than 7.5
+++ indicates a value from 5.0 up to 7.5 μM
++ indicates a value from 2.0 up to 5.0 μM
+ indicates a value less than 2.0 μM

All of the U.S. patents, U.S. patent application publications, U.S. patent applications, foreign patents, foreign patent applications and non-patent publications referred to in this specification are incorporated herein by reference in their entireties.

Although the foregoing disclosure has been described in some detail to facilitate understanding, it will be apparent that certain changes and modifications may be practiced within the scope of the appended claims. Accordingly, the described embodiments are to be considered as illustrative and not restrictive, and the disclosure is not to be limited to the details given herein, but may be modified within the scope and equivalents of the appended claims.

1. A compound of formula (I): 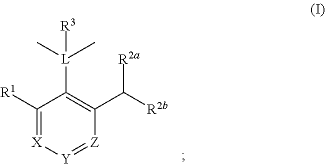
wherein:
X, Y, and Z are each independently N or CR^{1b}, provided that at least one and no more than two of X, Y, and Z are N;
L is a direct bond, —NR⁴C(═O)—, or —C(═O)NR⁴—;
R¹ is methoxy, —R⁵N(R⁶)₂, alkenyl, or has one of the following structures: 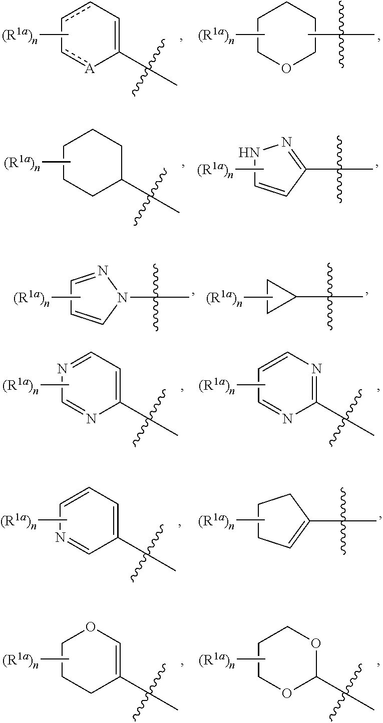
wherein:
each [symbol] is independently a single or double bond such that all valences are satisfied;
each R^{1a} is independently alkyl, halo, haloalkyl, —R⁵OR⁶, —R⁵N(R⁶)₂, —R⁵OC(═O)R⁶, optionally substituted cycloalkyl, or —R⁵C(═O)OR⁶;
A is O, N, or C;
each R^{1b} is independently hydrogen, halo, alkyl, or haloalkyl;
R^{2a} and R^{2b}, together with the carbon to which they are attached, form an optionally substituted cycloalkyl, an optionally substituted cycloalkenyl, an optionally substituted aryl, an optionally substituted heterocyclyl, or an optionally substituted heteroaryl;
or R^{2a} is hydrogen or alkyl and R^{2b} is an optionally substituted heterocyclyl or an optionally substituted cycloalkyl;
or R^{2a} and R^{2b} are both alkyl;
or R^{2a} is alkyl and R^{2b} is haloalkoxy;
R³ is alkyl, cyanoalkyl, —R⁵OR⁶, —R⁵N(R⁶)₂, an optionally substituted cycloalkyl, an optionally substituted aryl, an optionally substituted heterocyclyl, an optionally substituted heteroaryl, an optionally substituted cycloalkylalkyl, an optionally substituted heterocyclylalkyl;
R⁴ is hydrogen or alkyl;
each R⁵ is independently a direct bond or an optionally substituted alkylene chain;
each R⁶ is independently hydrogen, alkyl, haloalkyl, optionally substituted cycloalkyl, or optionally substituted arylalkyl;
or two R⁶'s, together with the nitrogen to which they are both attached, form an optionally substituted heterocyclyl; and
n is 0, 1, 2, 3, 4, or 5,
as a stereoisomer, enantiomer, or tautomer thereof or a mixture thereof, or a pharmaceutically acceptable salt, solvate, isotopologue, or prodrug thereof.

2. The compound of claim 1, wherein the compound has the following formula (Ia): 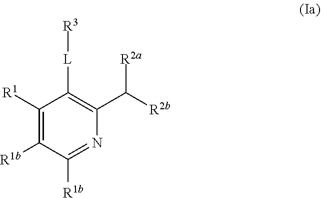
and R¹, R^{1b}, R^{2a}, R^{2b}, L and R³ are as defined in claim 1,
as a stereoisomer, enantiomer, or tautomer thereof or a mixture thereof, or a pharmaceutically acceptable salt, solvate, isotopologue, or prodrug thereof.

3. The compound of claim 1, wherein the compound has the following formula (Ia1): 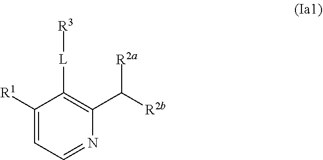
and R¹, R^{1b}, R^{2a}, R^{2b}, L and R³ are as defined in claim 1,
as a stereoisomer, enantiomer, or tautomer thereof or a mixture thereof, or a pharmaceutically acceptable salt, solvate, isotopologue, or prodrug thereof.

4. The compound of claim 1, wherein the compound has the following formula (Ib): 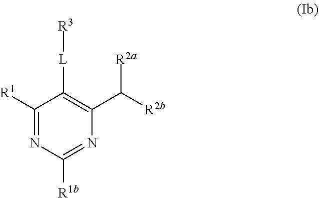
and R¹, R^{1b}, R^{2a}, R^{2b}, L and R³ are as defined in claim 1,
as a stereoisomer, enantiomer, or tautomer thereof or a mixture thereof, or a pharmaceutically acceptable salt, solvate, isotopologue, or prodrug thereof.

5. The compound of claim 1, wherein the compound has the following formula (Ib1): 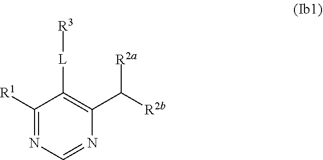
and R¹, R^{1b}, R^{2a}, R^{2b}, L and R³ are as defined in claim 1,
as a stereoisomer, enantiomer, or tautomer thereof or a mixture thereof; or a pharmaceutically acceptable salt, solvate, isotopologue, or prodrug thereof.

6. The compound of claim 1, wherein the compound has the following formula (Ic): 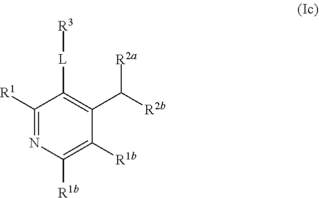
and R¹, R^{1b}, R^{2a}, R^{2b}, L and R³ are as defined in claim 1,
as a stereoisomer, enantiomer, or tautomer thereof or a mixture thereof; or a pharmaceutically acceptable salt, solvate, isotopologue, or prodrug thereof.

7. The compound of claim 1, wherein the compound has the following formula (Ic1): 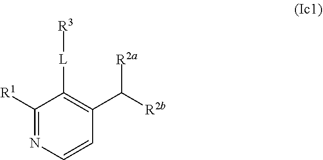
and R¹, R^{1b}, R^{2a}, R^{2b}, L and R³ are as defined in claim 1,
as a stereoisomer, enantiomer, or tautomer thereof or a mixture thereof, or a pharmaceutically acceptable salt, solvate, isotopologue, or prodrug thereof.

8. The compound of claim 1, wherein the compound has the following formula (Id): 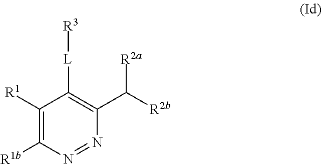
and R¹, R^{1b}, R^{2a}, R^{2b}, L and R³ are as defined in claim 1,
as a stereoisomer, enantiomer, or tautomer thereof or a mixture thereof, or a pharmaceutically acceptable salt, solvate, isotopologue, or prodrug thereof.

9. The compound of claim 1, wherein the compound has the following formula (Ie): 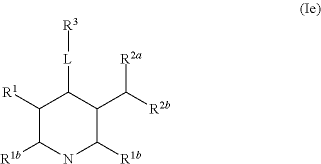
and R¹, R^{1b}, R^{2a}, R^{2b}, L and R³ are as defined in claim 1,
as a stereoisomer, enantiomer, or tautomer thereof or a mixture thereof, or a pharmaceutically acceptable salt, solvate, isotopologue, or prodrug thereof.

10. The compound of claim 1, wherein:
R¹ has one of the following structures: 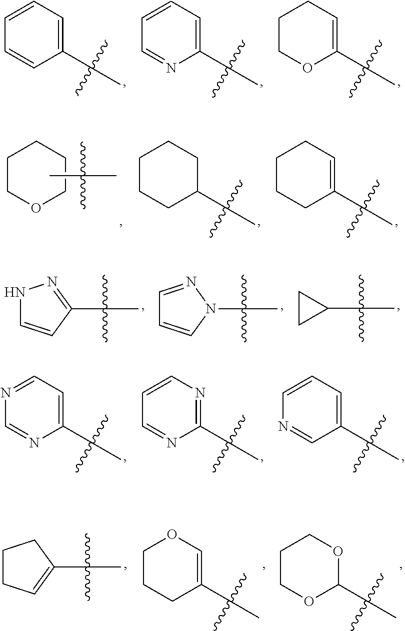
as a stereoisomer, enantiomer, or tautomer thereof or a mixture thereof, or a pharmaceutically acceptable salt, solvate, isotopologue, or prodrug thereof.

11. The compound of claim 1, wherein:
R¹ has one of the following structures: 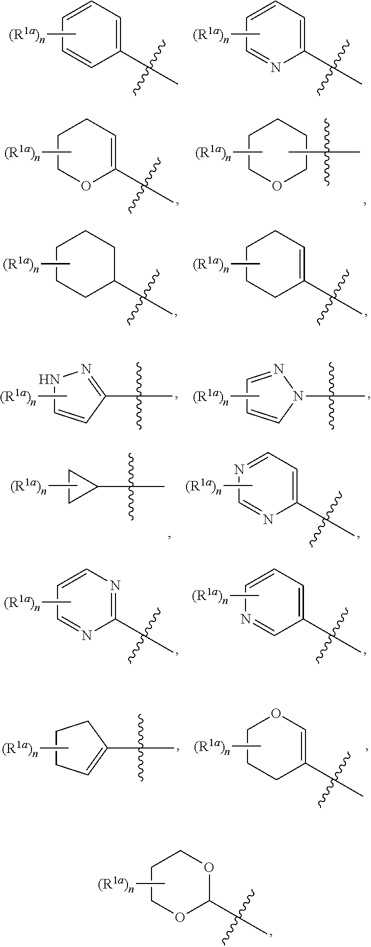
wherein:
n is 1, 2, 3, 4, or 5;
as a stereoisomer, enantiomer, or tautomer thereof or a mixture thereof, or a pharmaceutically acceptable salt, solvate, isotopologue, or prodrug thereof.

12. The compound of claim 11, wherein:
each R^{1a} is independently methyl, methoxy, trifluoromethyl, fluoro, chloro,
or has the following structure: 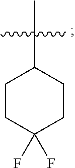 [SMILES: *C1CCC(F)(F)CC1]
as a stereoisomer, enantiomer, or tautomer thereof or a mixture thereof, or a pharmaceutically acceptable salt, solvate, isotopologue, or prodrug thereof.

13. The compound of claim 1, wherein:
R¹ has one of the following structures: 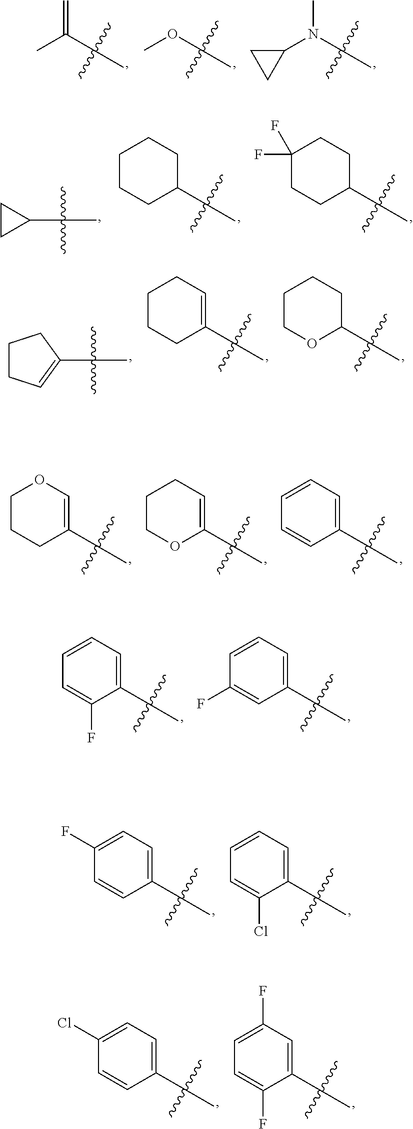 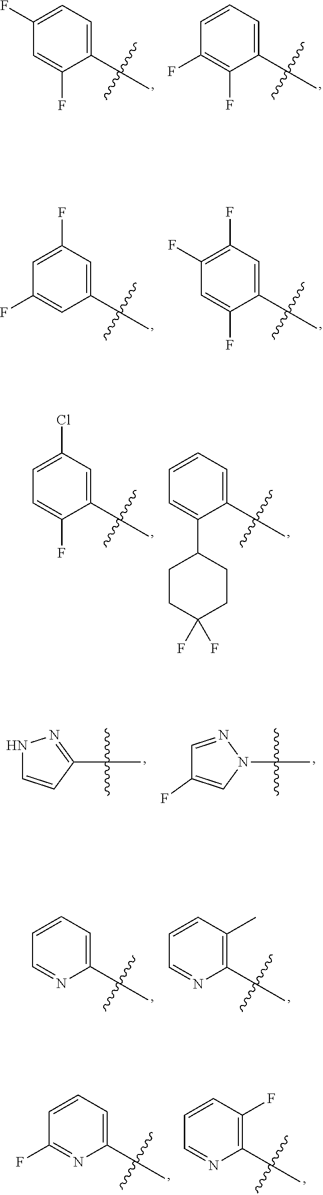 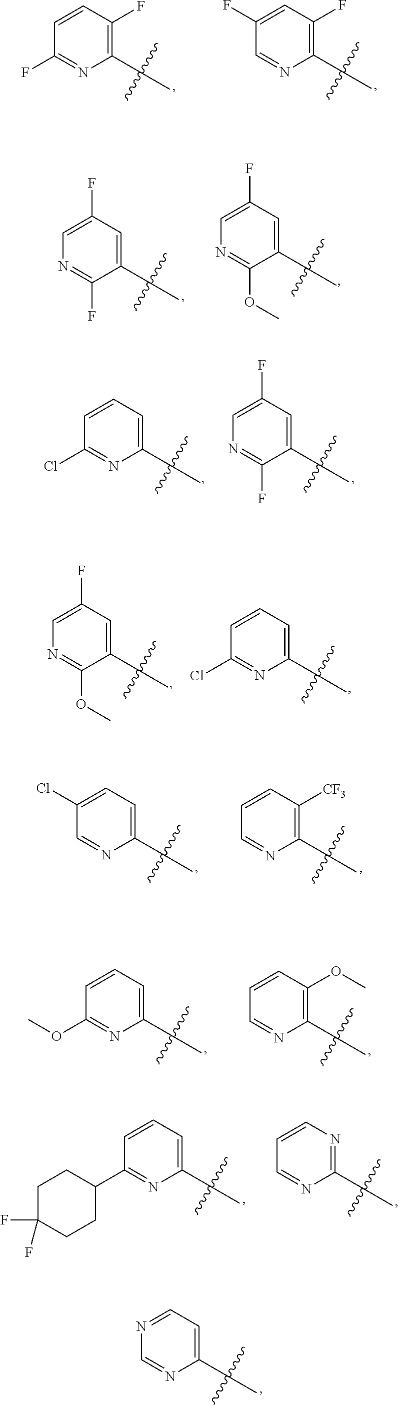
as a stereoisomer, enantiomer, or tautomer thereof or a mixture thereof, or a pharmaceutically acceptable salt, solvate, isotopologue, or prodrug thereof.

14. The compound of claim 1, wherein:
R¹ has one of the following structures: 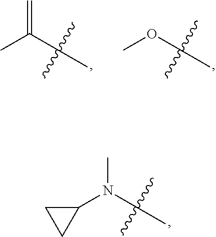
as a stereoisomer, enantiomer, or tautomer thereof or a mixture thereof, or a pharmaceutically acceptable salt, solvate, isotopologue, or prodrug thereof.

15. The compound of claim 1, wherein:
R¹ has one of the following structures: 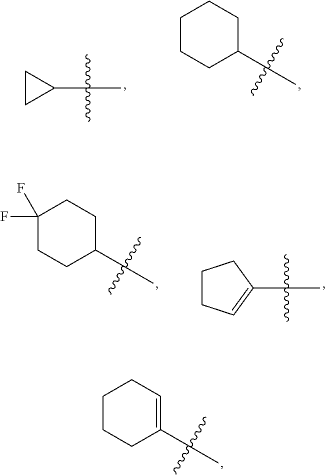
as a stereoisomer, enantiomer, or tautomer thereof or a mixture thereof, or a pharmaceutically acceptable salt, solvate, isotopologue, or prodrug thereof.

16. The compound of claim 1, wherein:
R¹ has one of the following structures: 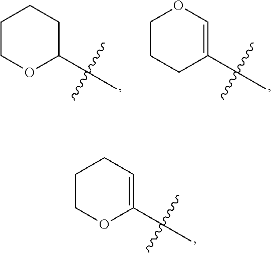 [SMILES: *C1CCCCO1 ; *C1=COCCC1 ; *C1=CCCCO1]
as a stereoisomer, enantiomer, or tautomer thereof or a mixture thereof; or a pharmaceutically acceptable salt, solvate, isotopologue, or prodrug thereof.

17. The compound of claim 1, wherein:
R¹ has one of the following structures: 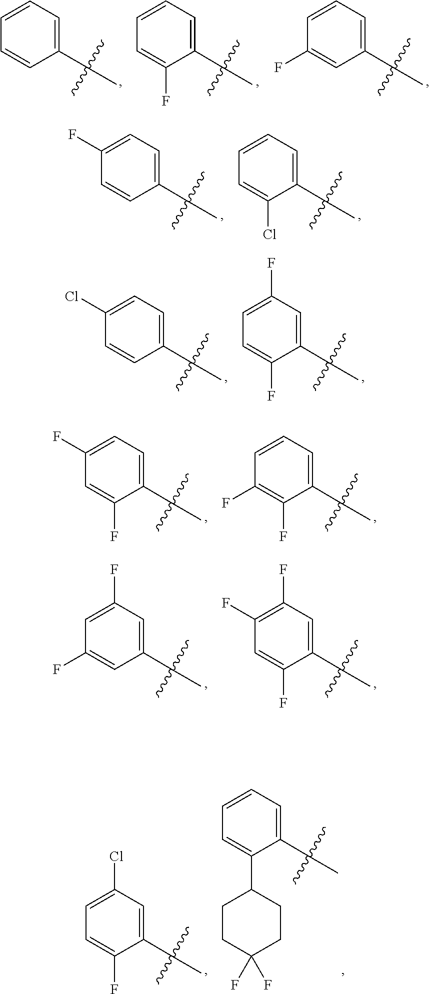
as a stereoisomer, enantiomer, or tautomer thereof or a mixture thereof, or a pharmaceutically acceptable salt, solvate, isotopologue, or prodrug thereof.

18. The compound of claim 1, wherein:
R¹ has one of the following structures: 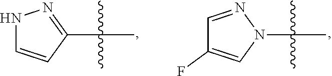 [SMILES: *c1cc[nH]n1 ; *n1cc(F)cn1]
as a stereoisomer, enantiomer, or tautomer thereof or a mixture thereof, or a pharmaceutically acceptable salt, solvate, isotopologue, or prodrug thereof.

19. The compound of claim 1, wherein:
R¹ has one of the following structures: 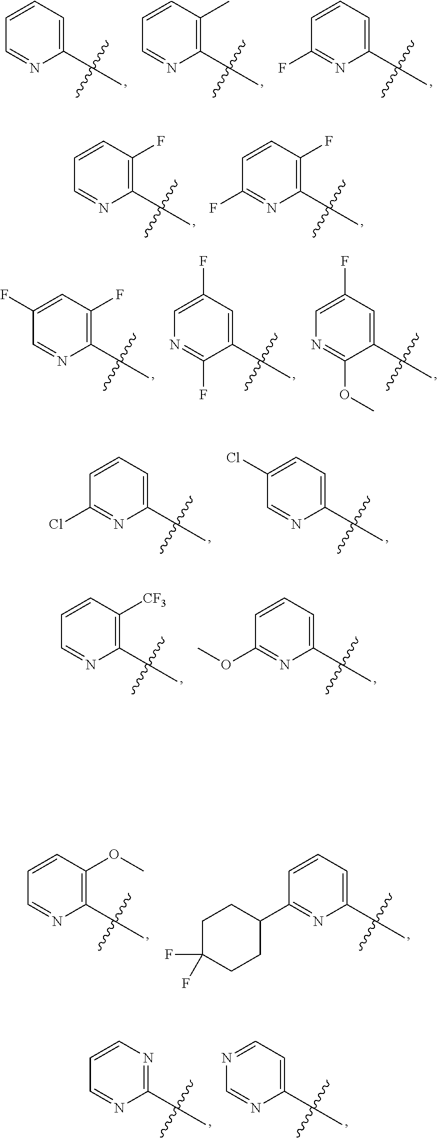
as a stereoisomer, enantiomer, or tautomer thereof or a mixture thereof, or a pharmaceutically acceptable salt, solvate, isotopologue, or prodrug thereof.

20. The compound of claim 1, wherein:
R^{2a} and R^{2b}, together with the carbon to which they are attached, form an optionally substituted cycloalkyl;
as a stereoisomer, enantiomer, or tautomer thereof or a mixture thereof, or a pharmaceutically acceptable salt, solvate, isotopologue, or prodrug thereof.

21. The compound of claim 1, wherein:
R^{2a} and R^{2b}, together with the carbon to which they are attached, form an optionally substituted monocyclic, fused, or spirocyclic cycloalkyl;
as a stereoisomer, enantiomer, or tautomer thereof or a mixture thereof, or a pharmaceutically acceptable salt, solvate, isotopologue, or prodrug thereof.

22. The compound of claim 1, wherein:
R^{2a} and R^{2b}, together with the carbon to which they are attached, form an optionally substituted cycloalkenyl;
as a stereoisomer, enantiomer, or tautomer thereof or a mixture thereof, or a pharmaceutically acceptable salt, solvate, isotopologue, or prodrug thereof.

23. The compound of claim 1, wherein:
R^{2a} and R^{2b}, together with the carbon to which they are attached, form an optionally substituted aryl;
as a stereoisomer, enantiomer, or tautomer thereof or a mixture thereof, or a pharmaceutically acceptable salt, solvate, isotopologue, or prodrug thereof.

24. The compound of claim 1, wherein:
R^{2a} and R^{2b}, together with the carbon to which they are attached, form an optionally substituted heterocyclyl;
as a stereoisomer, enantiomer, or tautomer thereof or a mixture thereof, or a pharmaceutically acceptable salt, solvate, isotopologue, or prodrug thereof.

25. The compound of claim 1, wherein:
R^{2a} and R^{2b}, together with the carbon to which they are attached, form an optionally substituted N-heterocyclyl;
as a stereoisomer, enantiomer, or tautomer thereof or a mixture thereof, or a pharmaceutically acceptable salt, solvate, isotopologue, or prodrug thereof.

26. The compound of claim 1, wherein:
R^{2a} and R^{2b}, together with the carbon to which they are attached, form an optionally substituted monocyclic N-heterocyclyl;
as a stereoisomer, enantiomer, or tautomer thereof or a mixture thereof, or a pharmaceutically acceptable salt, solvate, isotopologue, or prodrug thereof.

27. The compound of claim 1, wherein:
R^{2a} and R^{2b}, together with the carbon to which they are attached, form an optionally substituted O-heterocyclyl;
as a stereoisomer, enantiomer, or tautomer thereof or a mixture thereof, or a pharmaceutically acceptable salt, solvate, isotopologue, or prodrug thereof.

28. The compound of claim 1, wherein:
R^{2a} and R^{2b}, together with the carbon to which they are attached, form an optionally substituted monocyclic or fused O-heterocyclyl;
as a stereoisomer, enantiomer, or tautomer thereof or a mixture thereof, or a pharmaceutically acceptable salt, solvate, isotopologue, or prodrug thereof.

29. The compound of claim 1, wherein:
R^{2a} and R^{2b}, together with the carbon to which they are attached, form an optionally substituted heteroaryl;
as a stereoisomer, enantiomer, or tautomer thereof or a mixture thereof, or a pharmaceutically acceptable salt, solvate, isotopologue, or prodrug thereof.

30. The compound of claim 1, wherein:
R^{2a} hydrogen or alkyl and R^{2b} is an optionally substituted heterocyclyl or an optionally substituted cycloalkyl;
as a stereoisomer, enantiomer, or tautomer thereof or a mixture thereof, or a pharmaceutically acceptable salt, solvate, isotopologue, or prodrug thereof.

31. The compound of claim 1, wherein:
R^{2a} and R^{2b} are both alkyl;
as a stereoisomer, enantiomer, or tautomer thereof or a mixture thereof, or a pharmaceutically acceptable salt, solvate, isotopologue, or prodrug thereof.

32. The compound of claim 1, wherein:
R^{2a} is alkyl and R^{2b} is haloalkoxy;
as a stereoisomer, enantiomer, or tautomer thereof or a mixture thereof, or a pharmaceutically acceptable salt, solvate, isotopologue, or prodrug thereof.

33. The compound of claim 1, wherein:
R^{2a} and R^{2b}, together with the carbon to which they are attached, form one of the following structures: 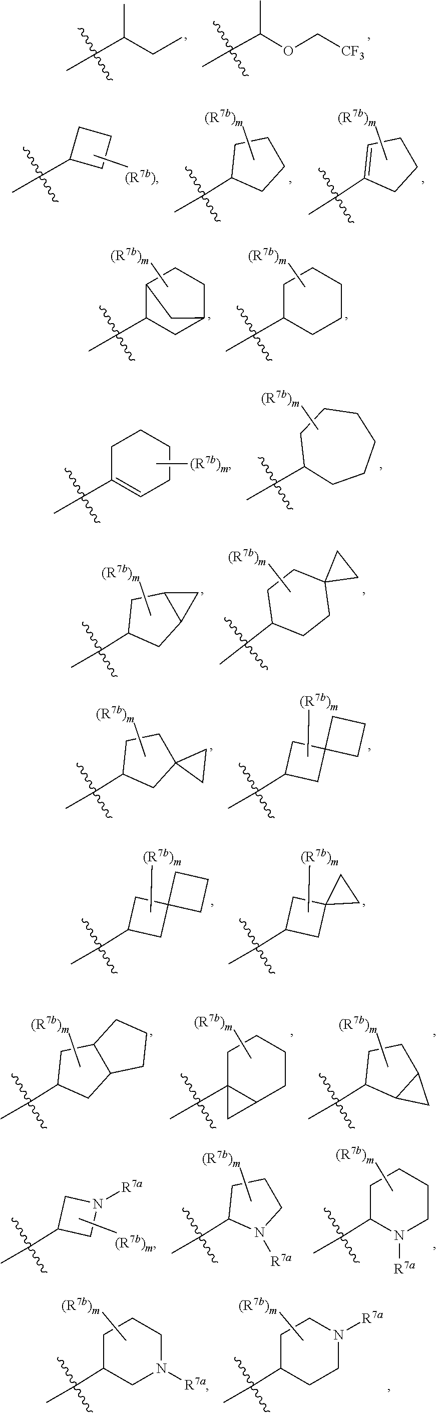 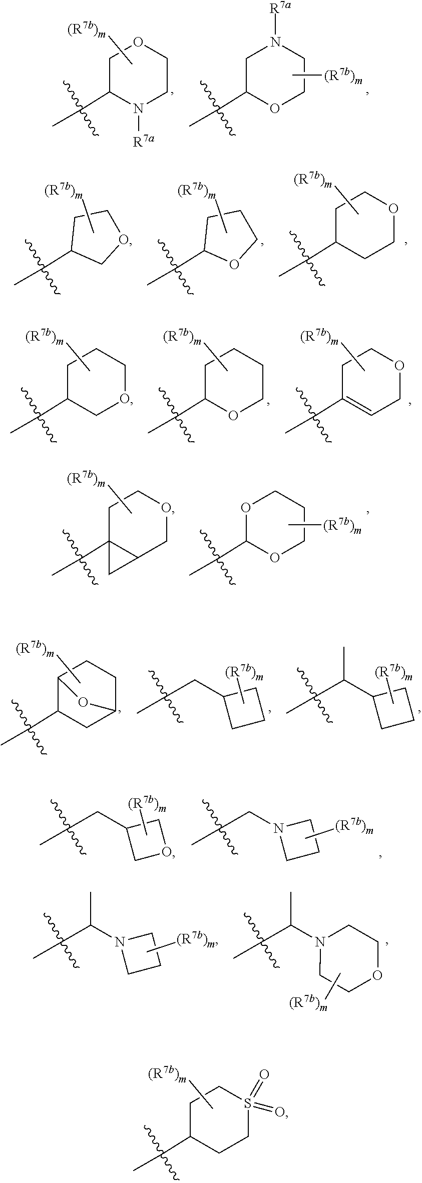
R^{7a} is hydrogen, alkyl, haloalkyl, —R^{7c}C(═O)R^{7d}, —R^{7c}C(═O)OR^{7d}, or heterocyclyl;
each R^{7b} is independently alkyl, halo, haloalkyl, cyano, —R^{7c}OR^{7d}, or —R^{7c}OC(═O)R^{7d}, or two R^{7b}'s, together with the carbon to which they are both attached join to form —C(═O)—;
each R^{7c} is independently a direct bond or an optionally substituted alkylene chain;
each R^{7d} is independently hydrogen, alkyl, haloalkyl, optionally substituted cycloalkyl, or optionally substituted arylalkyl;
or two R^{7d}'s, together with the nitrogen to which they are both attached, form an optionally substituted heterocyclyl; and
m is 0, 1, 2, 3, 4, or 5,
as a stereoisomer, enantiomer, or tautomer thereof or a mixture thereof, or a pharmaceutically acceptable salt, solvate, isotopologue, or prodrug thereof.

34. The compound of claim 1, wherein:
R^{2a} and R^{2b}, together with the carbon to which they are attached, form one of the following structures: 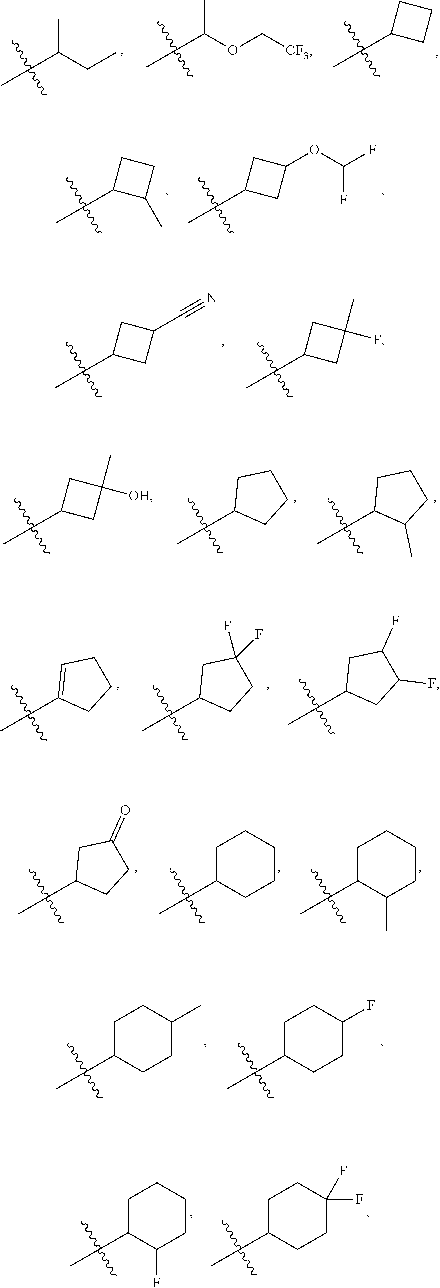 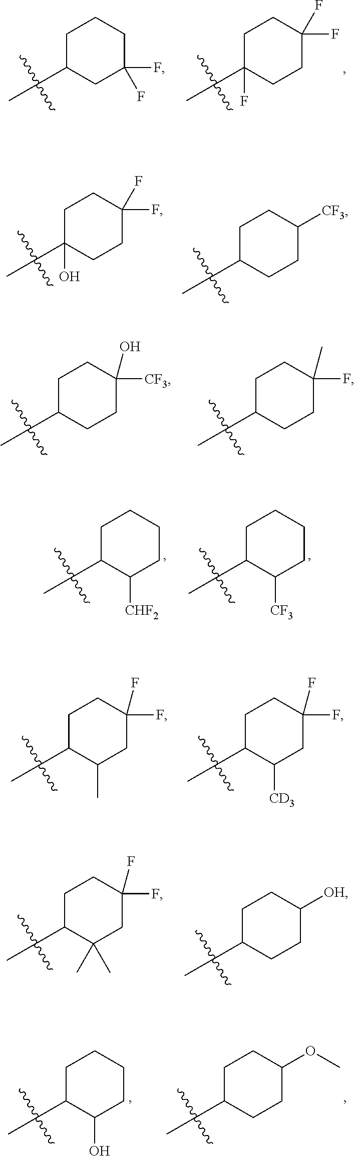 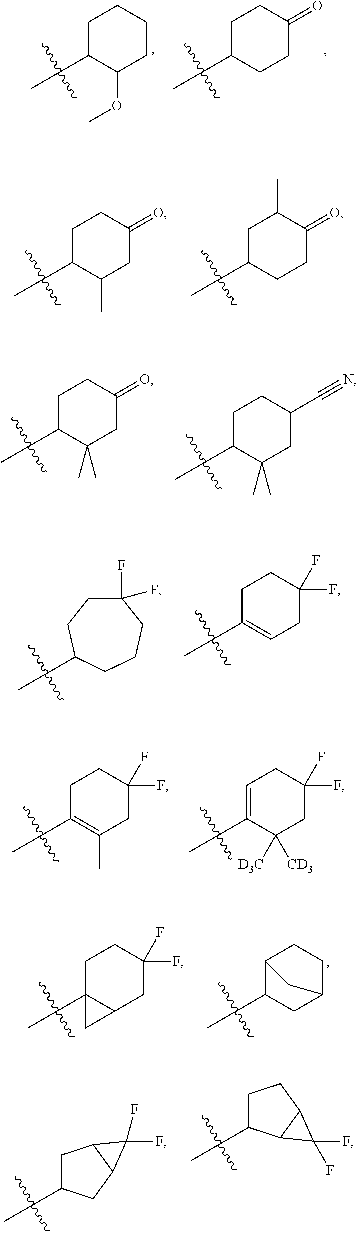 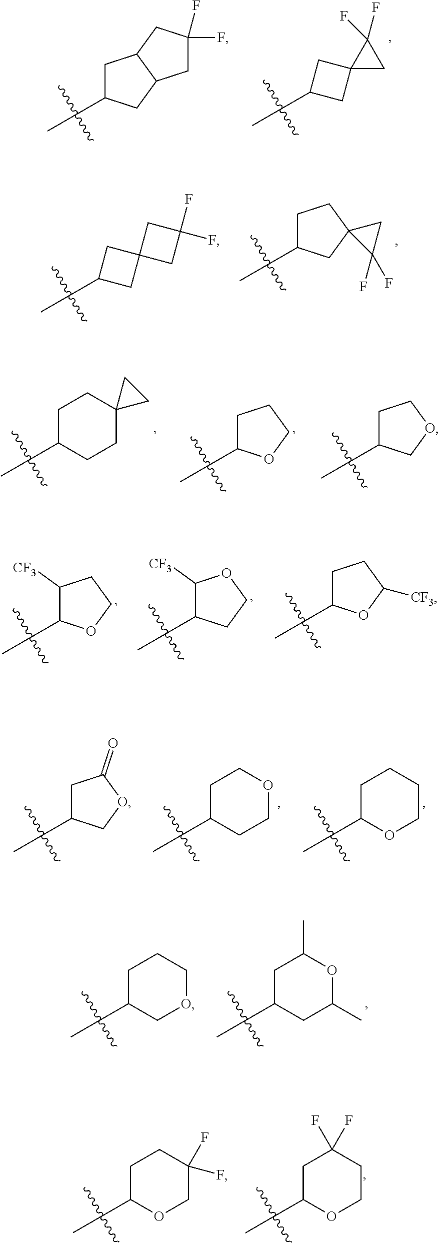 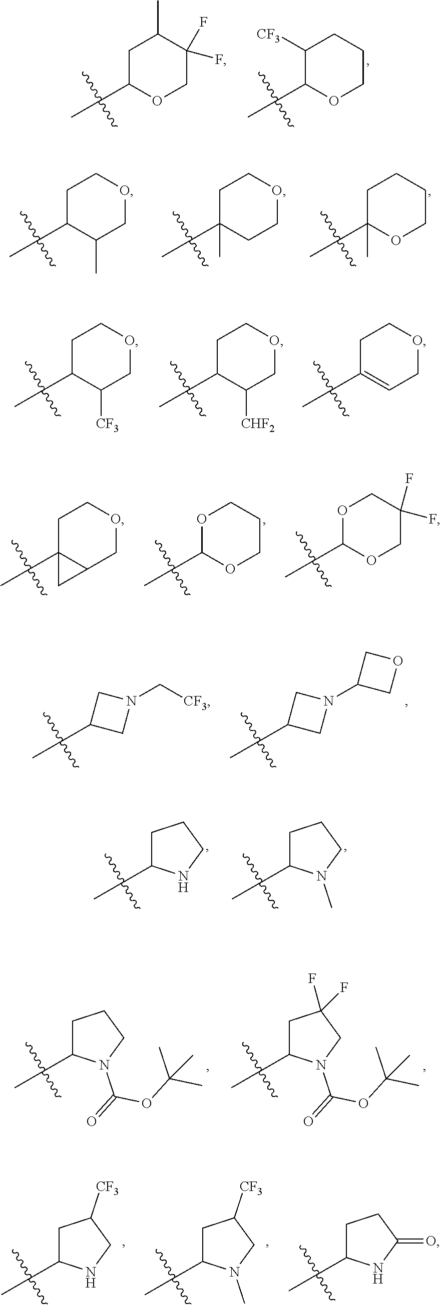 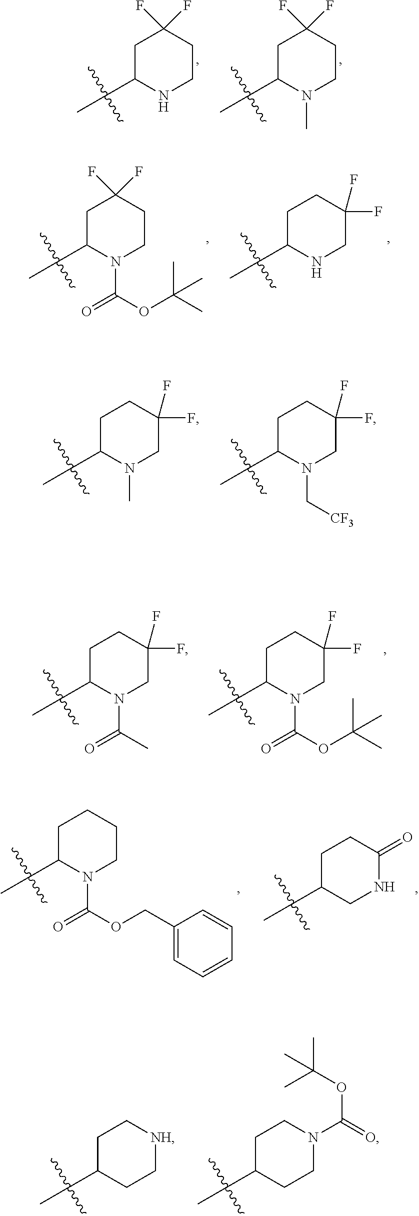 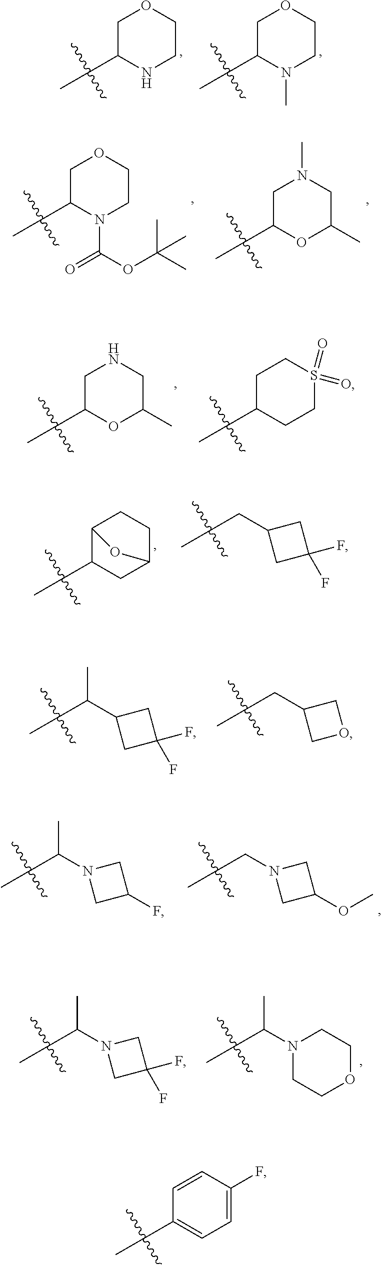
as a stereoisomer, enantiomer, or tautomer thereof or a mixture thereof; or a pharmaceutically acceptable salt, solvate, isotopologue, or prodrug thereof.

35. The compound of claim 1, wherein:
R^{2a} and R^{2b}, together with the carbon to which they are attached, form one of the following structures: 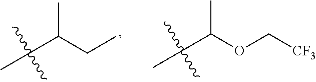
as a stereoisomer, enantiomer, or tautomer thereof or a mixture thereof; or a pharmaceutically acceptable salt, solvate, isotopologue, or prodrug thereof.

36. The compound of claim 1, wherein:
R^{2a} and R^{2b}, together with the carbon to which they are attached, form one of the following structures: 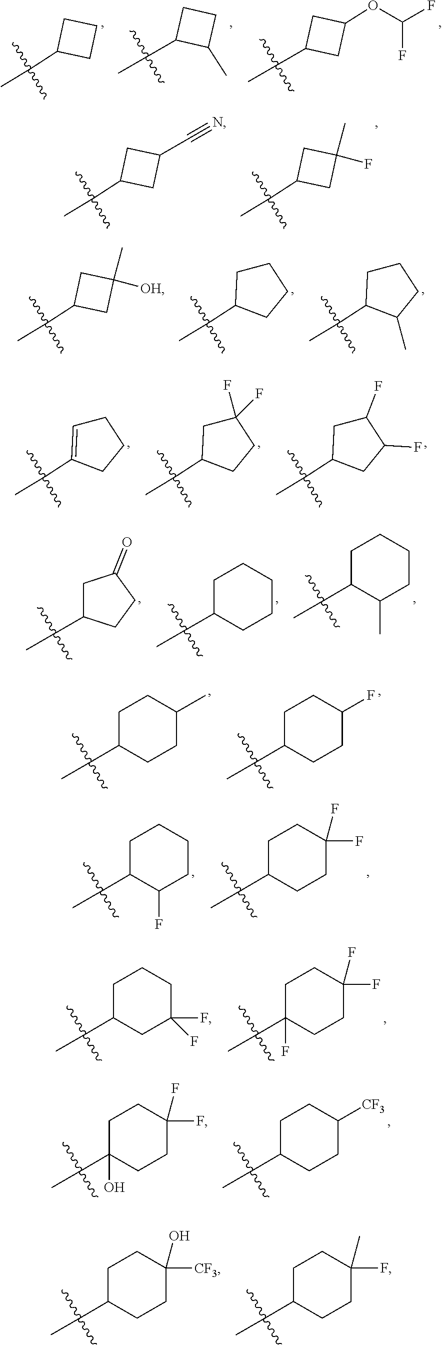 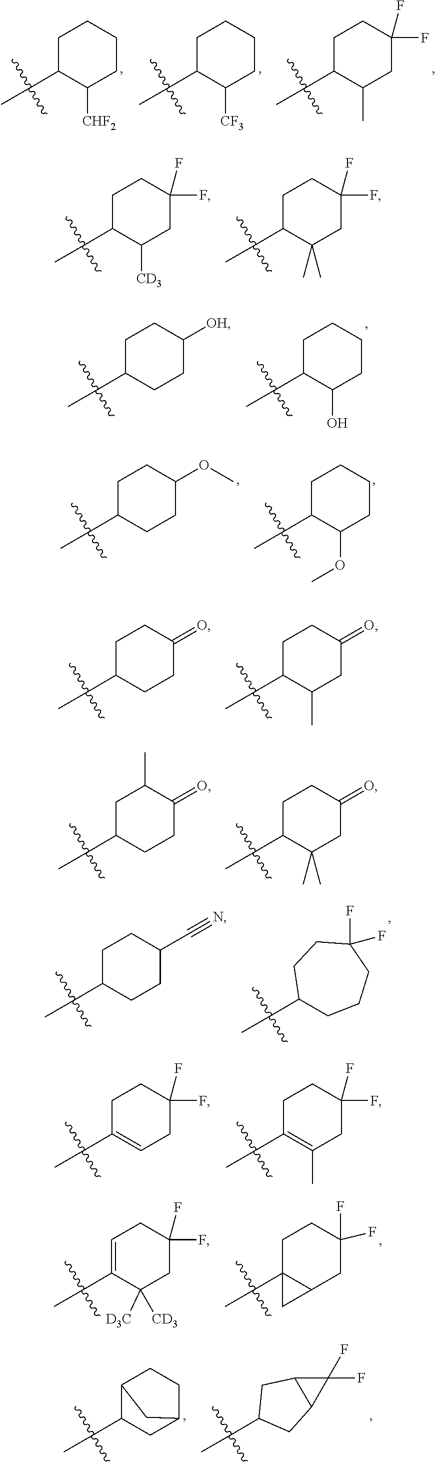 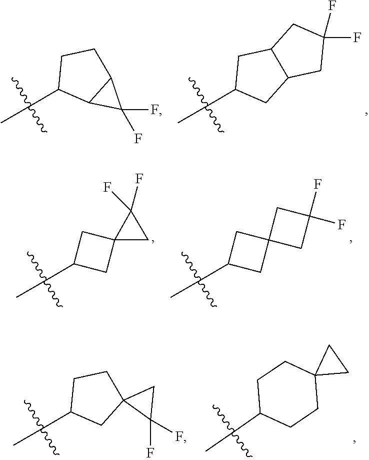
as a stereoisomer, enantiomer, or tautomer thereof or a mixture thereof; or a pharmaceutically acceptable salt, solvate, isotopologue, or prodrug thereof.

37. The compound of claim 1, wherein:
R^{2a} and R^{2b}, together with the carbon to which they are attached, form one of the following structures: 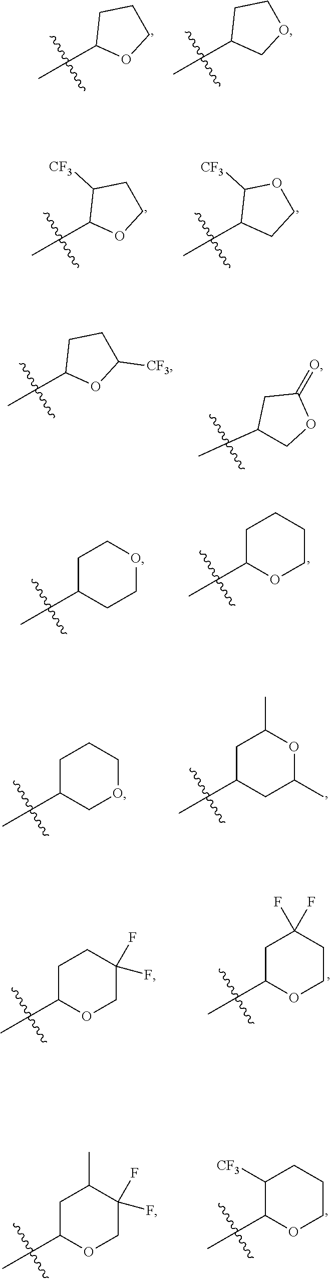 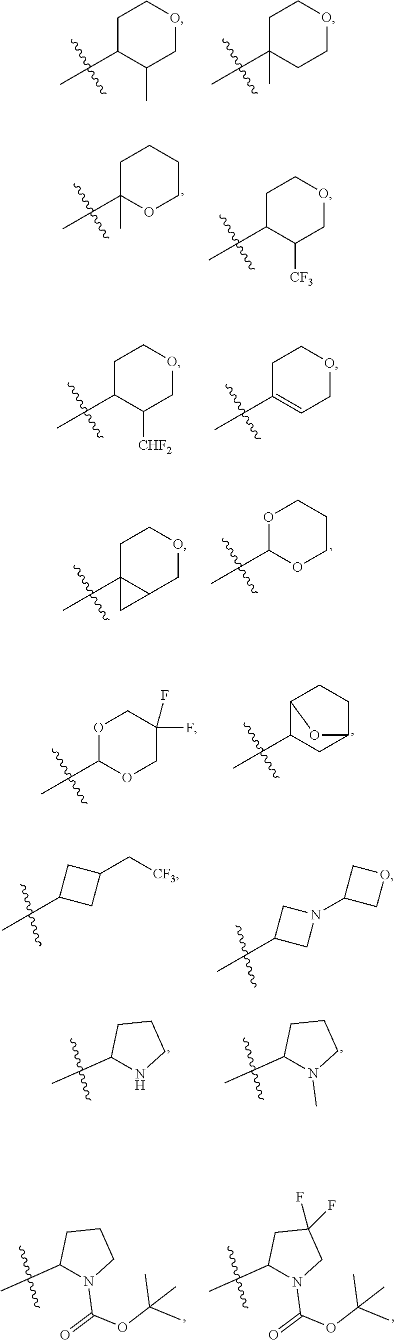 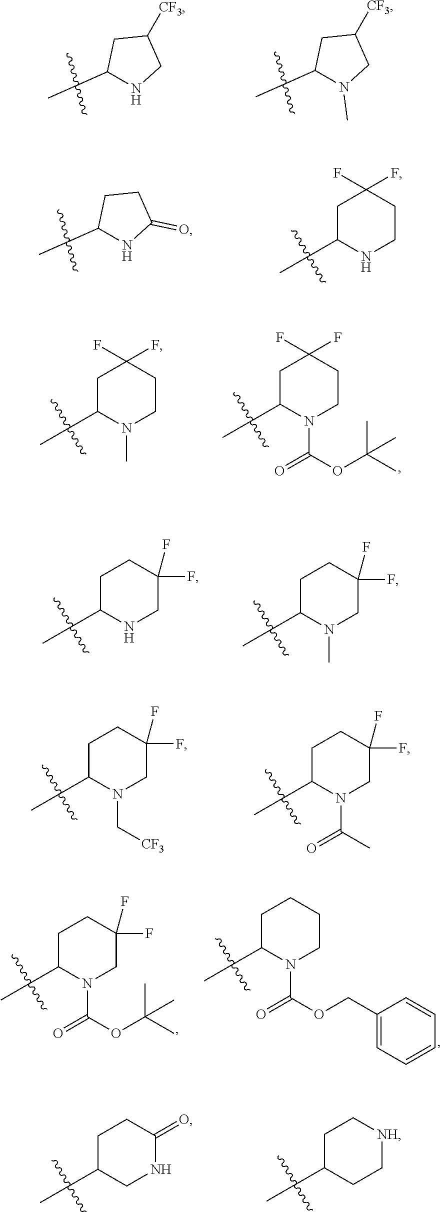 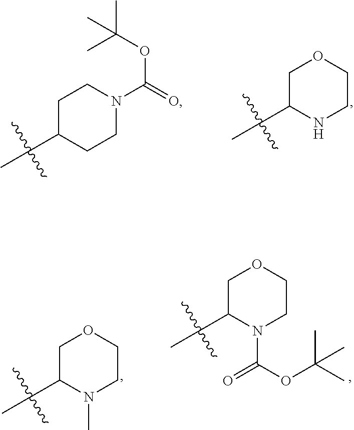
as a stereoisomer, enantiomer, or tautomer thereof or a mixture thereof, or a pharmaceutically acceptable salt, solvate, isotopologue, or prodrug thereof.

38. The compound of claim 1, wherein:
R^{2a} and R^{2b}, together with the carbon to which they are attached, form one of the following structures: 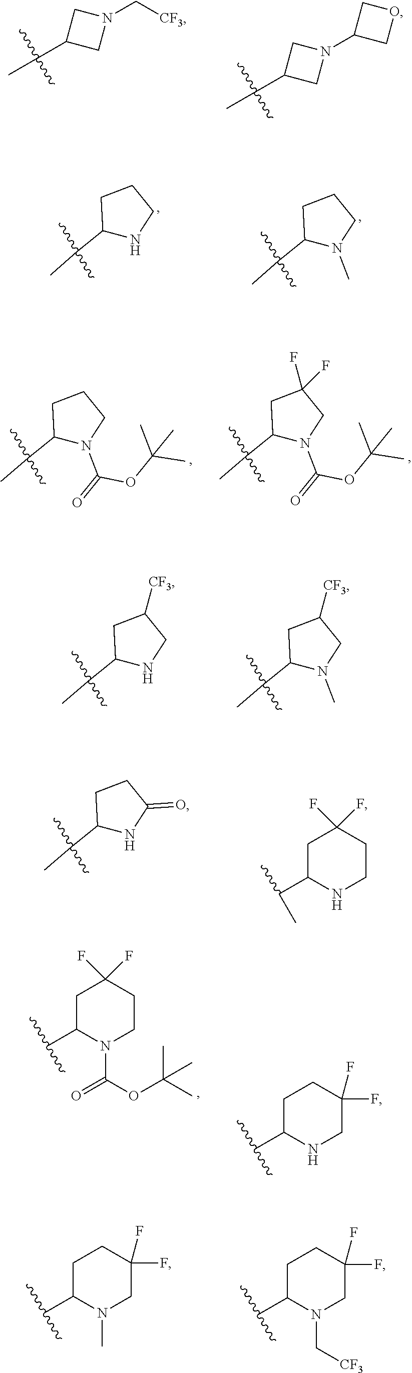 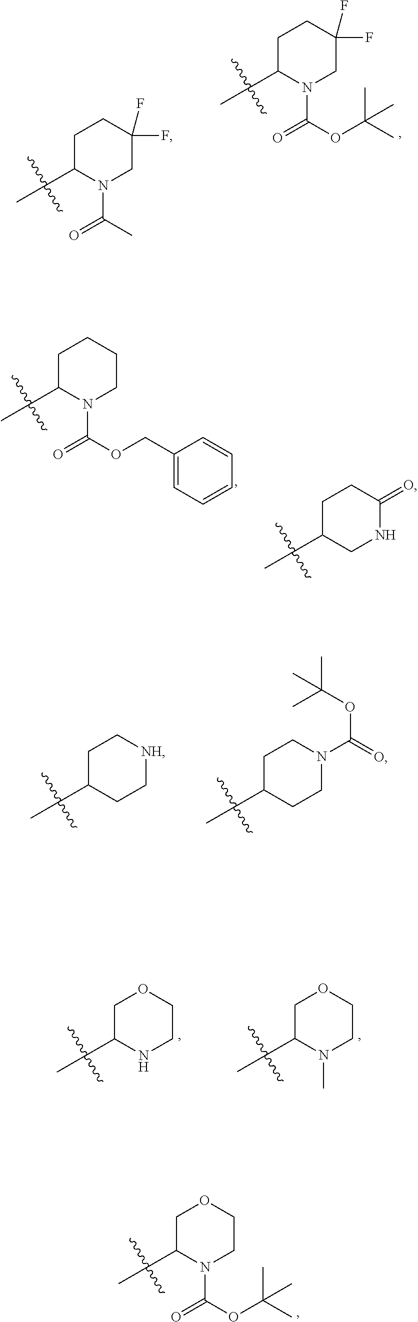
as a stereoisomer, enantiomer, or tautomer thereof or a mixture thereof; or a pharmaceutically acceptable salt, solvate, isotopologue, or prodrug thereof.

39. The compound of claim 1, wherein:
R^{2a} and R^{2b}, together with the carbon to which they are attached, form one of the following structures: 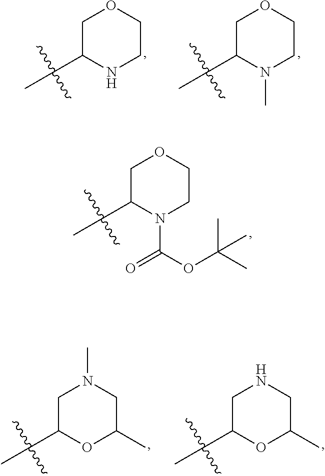
as a stereoisomer, enantiomer, or tautomer thereof or a mixture thereof, or a pharmaceutically acceptable salt, solvate, isotopologue, or prodrug thereof.

40. The compound of claim 1, wherein:
R^{2a} and R^{2b}, together with the carbon to which they are attached, form one of the following structures: 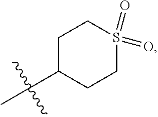 [SMILES: *C1CCS(=O)(=O)CC1]
as a stereoisomer, enantiomer, or tautomer thereof or a mixture thereof, or a pharmaceutically acceptable salt, solvate, isotopologue, or prodrug thereof.

41. The compound of claim 1, wherein:
R^{2a} and R^{2b}, together with the carbon to which they are attached, form one of the following structures: 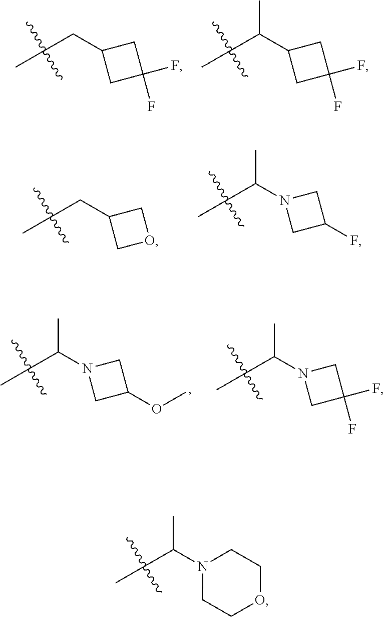
as a stereoisomer, enantiomer, or tautomer thereof or a mixture thereof; or a pharmaceutically acceptable salt, solvate, isotopologue, or prodrug thereof.

42. The compound of claim 1, wherein:
R^{2a} and R^{2b}, together with the carbon to which they are attached, form one of the following structures: 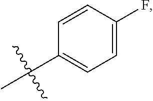 [SMILES: *c1ccc(F)cc1]
as a stereoisomer, enantiomer, or tautomer thereof or a mixture thereof, or a pharmaceutically acceptable salt, solvate, isotopologue, or prodrug thereof.

43. The compound of claim 1, wherein:
R³ is alkyl;
as a stereoisomer, enantiomer, or tautomer thereof or a mixture thereof, or a pharmaceutically acceptable salt, solvate, isotopologue, or prodrug thereof.

44. The compound of claim 1, wherein:
R³ is cyanoalkyl;
as a stereoisomer, enantiomer, or tautomer thereof or a mixture thereof, or a pharmaceutically acceptable salt, solvate, isotopologue, or prodrug thereof.

45. The compound of claim 1, wherein:
R³ is —R⁵OR⁶;
as a stereoisomer, enantiomer, or tautomer thereof or a mixture thereof, or a pharmaceutically acceptable salt, solvate, isotopologue, or prodrug thereof.

46. The compound of claim 1, wherein:
R³ is —R⁵N(R⁶)₂;
as a stereoisomer, enantiomer, or tautomer thereof or a mixture thereof, or a pharmaceutically acceptable salt, solvate, isotopologue, or prodrug thereof.

47. The compound of claim 1, wherein:
R³ is an optionally substituted cycloalkyl;
as a stereoisomer, enantiomer, or tautomer thereof or a mixture thereof, or a pharmaceutically acceptable salt, solvate, isotopologue, or prodrug thereof.

48. The compound of claim 1, wherein:
R³ is an optionally substituted aryl;
as a stereoisomer, enantiomer, or tautomer thereof or a mixture thereof, or a pharmaceutically acceptable salt, solvate, isotopologue, or prodrug thereof.

49. The compound of claim 1, wherein:
R³ is an optionally substituted heterocyclyl;
as a stereoisomer, enantiomer, or tautomer thereof or a mixture thereof, or a pharmaceutically acceptable salt, solvate, isotopologue, or prodrug thereof.

50. The compound of claim 1, wherein:
R³ is an optionally substituted N-heterocyclyl;
as a stereoisomer, enantiomer, or tautomer thereof or a mixture thereof, or a pharmaceutically acceptable salt, solvate, isotopologue, or prodrug thereof.

51. The compound of claim 1, wherein:
R³ is an optionally substituted heteroaryl;
as a stereoisomer, enantiomer, or tautomer thereof or a mixture thereof, or a pharmaceutically acceptable salt, solvate, isotopologue, or prodrug thereof.

52. The compound of claim 1, wherein:
R³ is an optionally substituted N-heteroaryl;
as a stereoisomer, enantiomer, or tautomer thereof or a mixture thereof, or a pharmaceutically acceptable salt, solvate, isotopologue, or prodrug thereof.

53. The compound of claim 1, wherein:
R³ is an optionally substituted 5- or 6-membered heteroaryl;
as a stereoisomer, enantiomer, or tautomer thereof or a mixture thereof, or a pharmaceutically acceptable salt, solvate, isotopologue, or prodrug thereof.

54. The compound of claim 1, wherein:
R³ is an optionally substituted fused bicyclic heteroaryl;
as a stereoisomer, enantiomer, or tautomer thereof or a mixture thereof, or a pharmaceutically acceptable salt, solvate, isotopologue, or prodrug thereof.

55. The compound of claim 1, wherein:
R³ is an optionally substituted cycloalkylalkyl;
as a stereoisomer, enantiomer, or tautomer thereof or a mixture thereof, or a pharmaceutically acceptable salt, solvate, isotopologue, or prodrug thereof.

56. The compound of claim 1, wherein:
R³ is an optionally substituted heterocyclylalkyl;
as a stereoisomer, enantiomer, or tautomer thereof or a mixture thereof, or a pharmaceutically acceptable salt, solvate, isotopologue, or prodrug thereof.

57. The compound of claim 1, wherein:
R³ has one of the following structures: 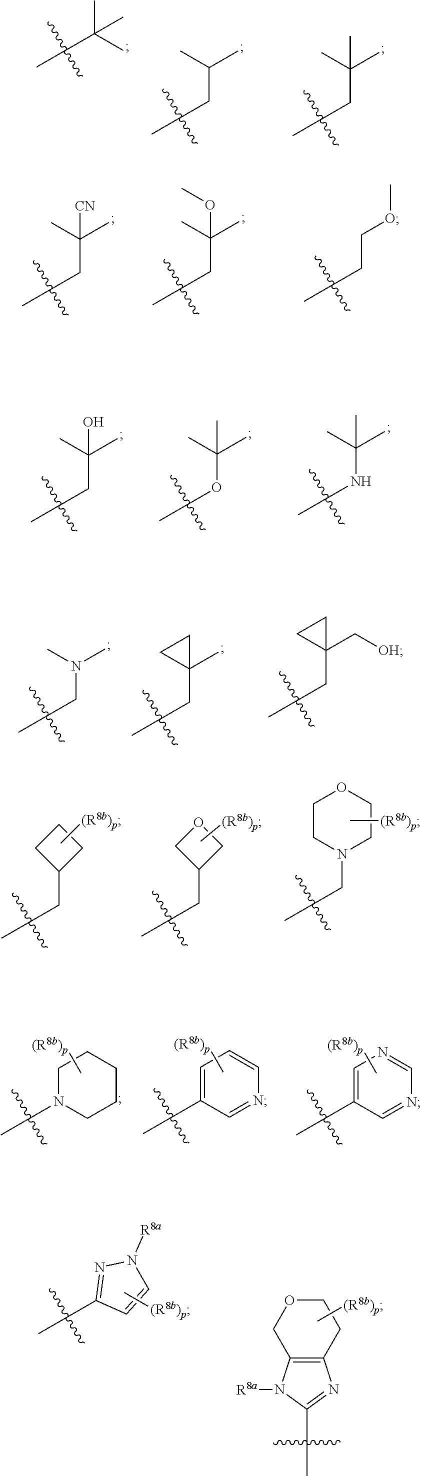 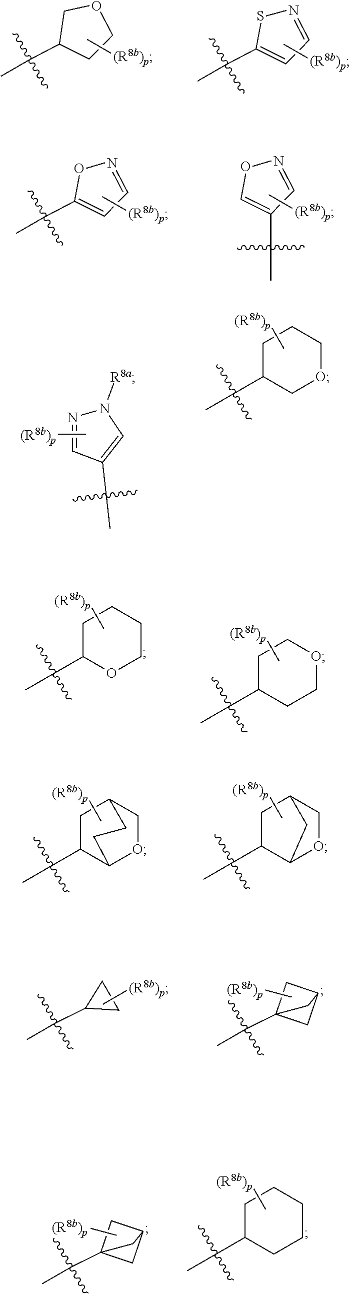 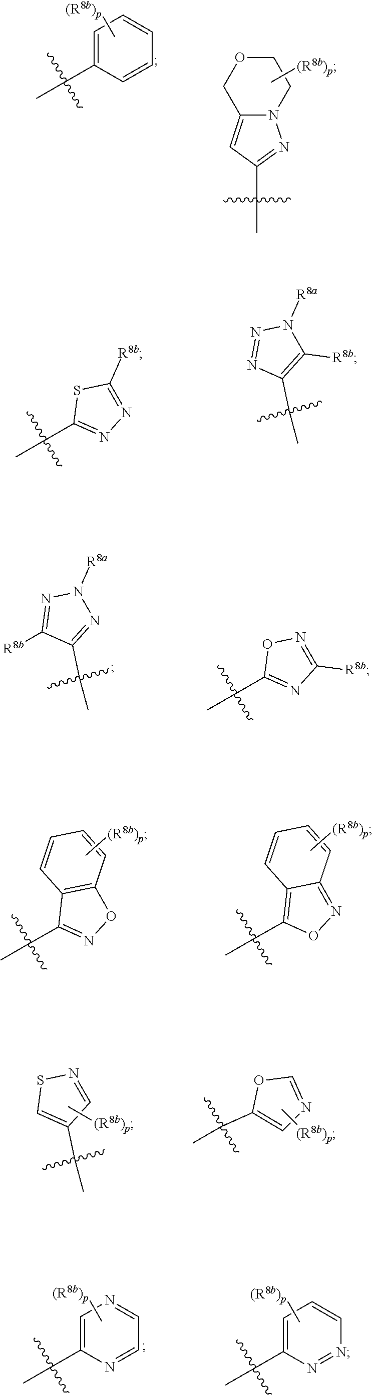 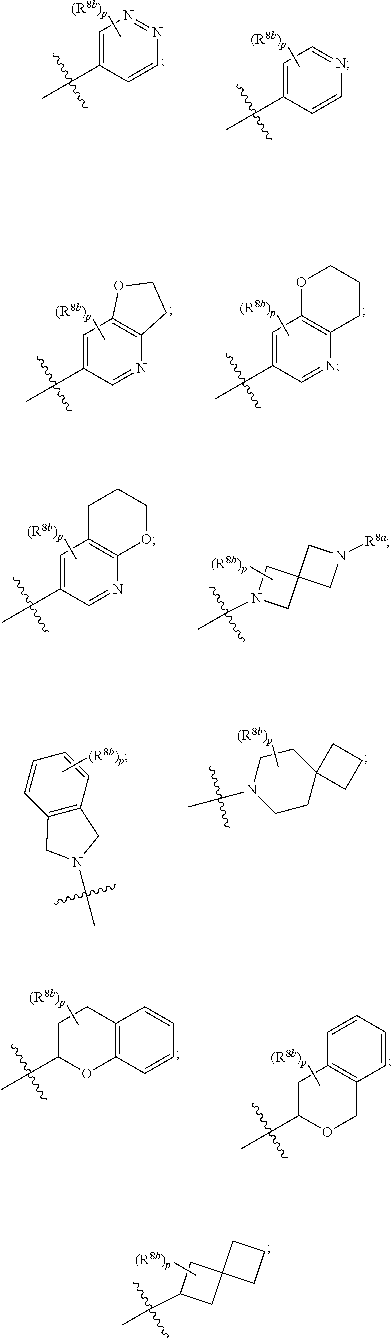
wherein:
R^{8a} is hydrogen, alkyl, haloalkyl, —C(═O)OR^{8d}, optionally substituted aryl, optionally substituted heterocyclylalkyl, optionally substituted cycloalkylalkyl, or optionally substituted cycloalkyl;
each R^{8b} is independently alkyl, optionally substituted cycloalkyl, cyano, halo, —R^{8c}OR^{8d}, —OR^{8c}N(R^{8d})₂, —C(═O)N(R^{8d})₂, —R^{8c}N(R^{8d})₂, or optionally substituted heterocyclyl,
each R^{8c} is independently a direct bond or an optionally substituted alkylene chain;
each R^{8d} is independently hydrogen, alkyl, haloalkyl, hydroxyalkyl, alkoxyalkyl, haloalkoxyalkyl, optionally substituted heterocyclyl, optionally substituted heterocyclylalkyl, optionally substituted cycloalkyl, or optionally substituted cycloalkylalkyl;
or two R^{8d}'s, together with the nitrogen to which they are both attached, form an optionally substituted heterocyclyl;
p is 0, 1, 2, 3, 4, or 5; and
as a stereoisomer, enantiomer, or tautomer thereof or a mixture thereof; or a pharmaceutically acceptable salt, solvate, isotopologue, or prodrug thereof.

58. The compound of claim 1, wherein:
R³ has one of the following structures: 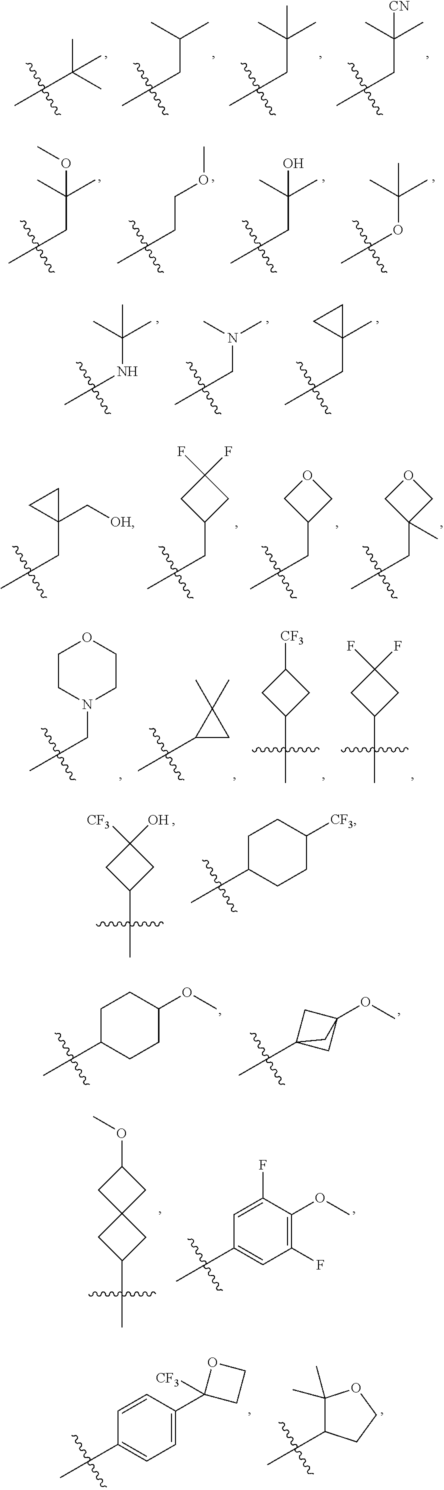 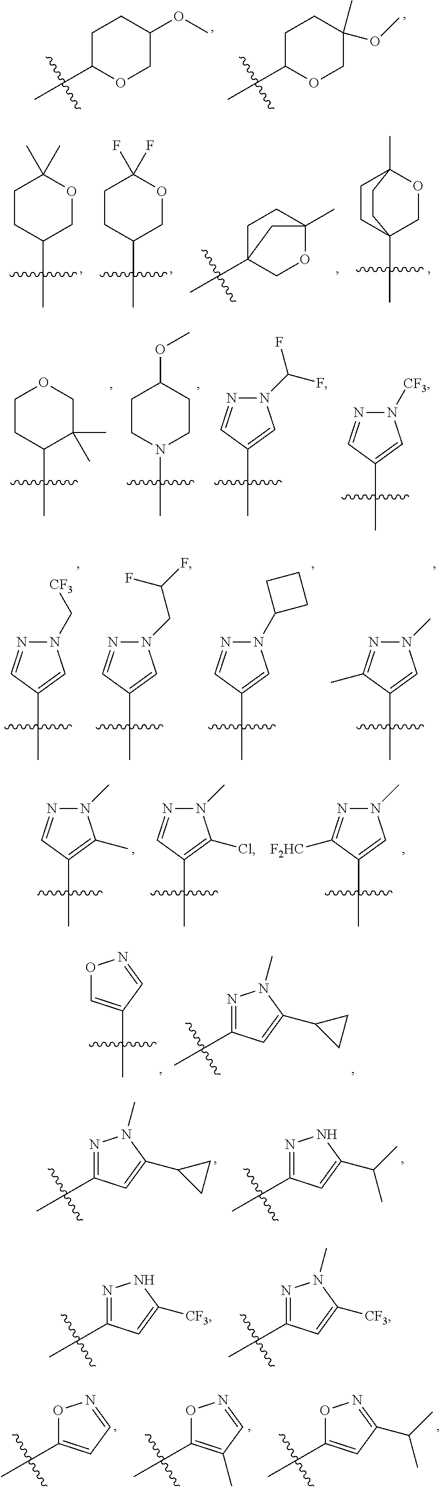 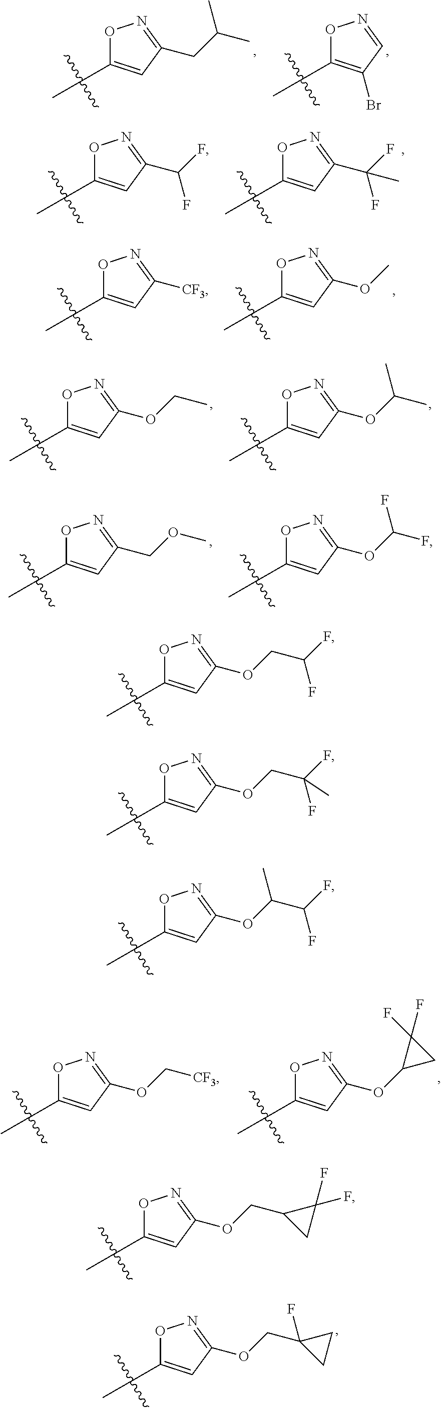 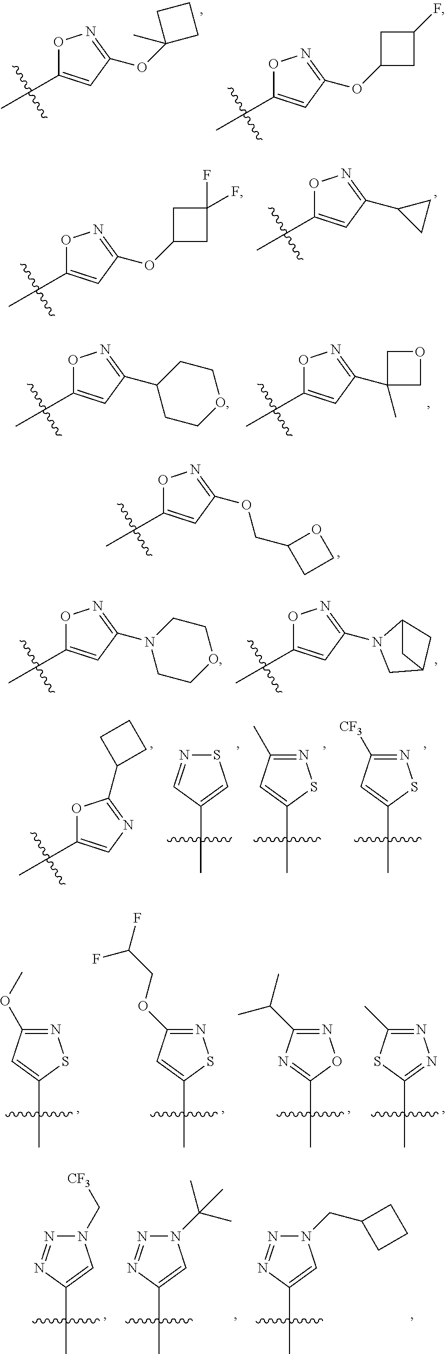 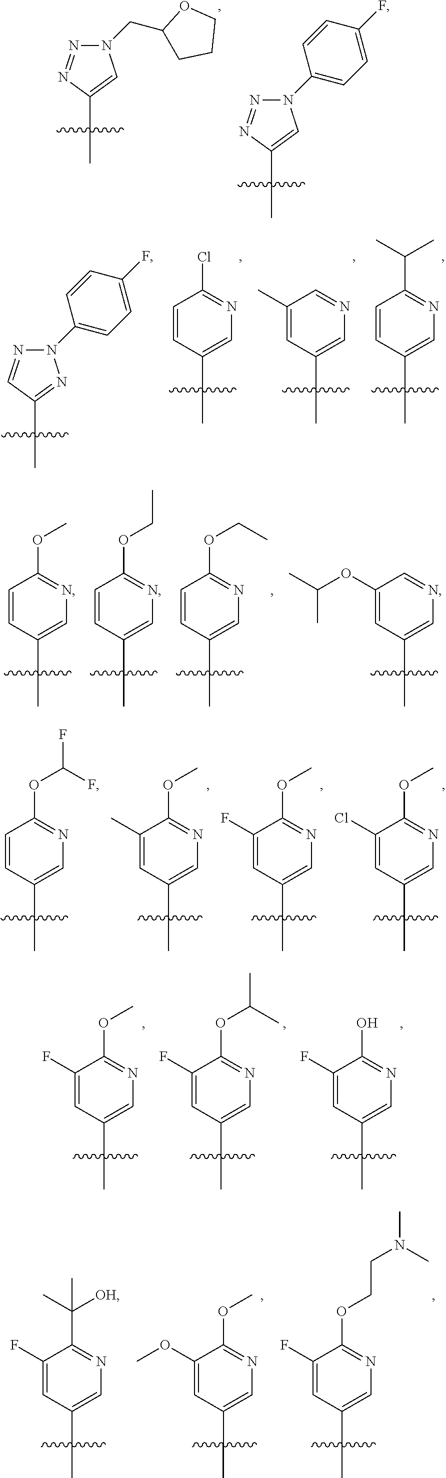 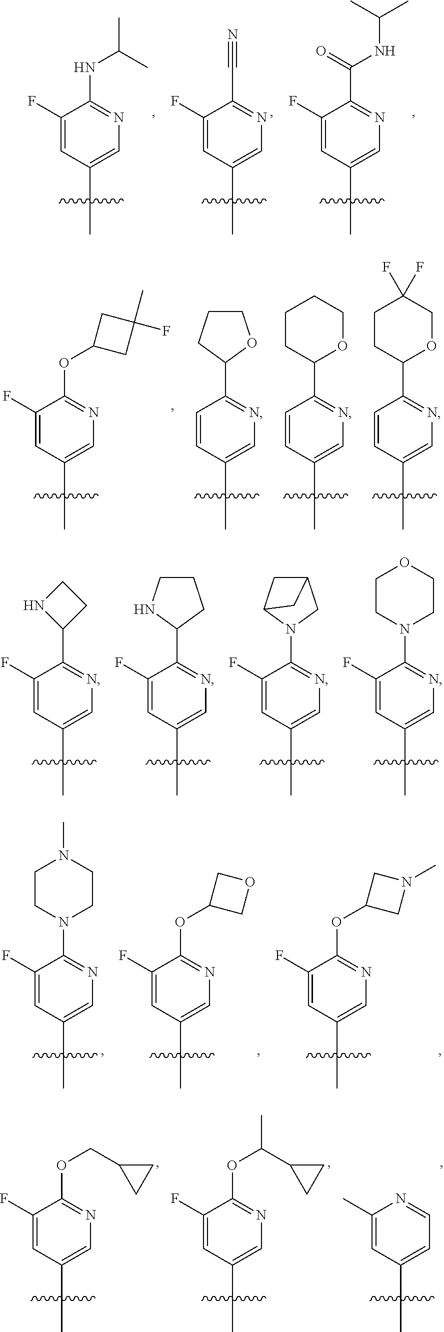 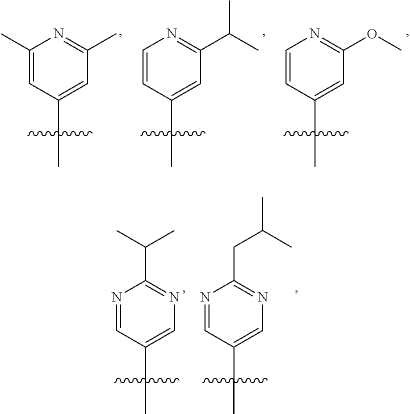 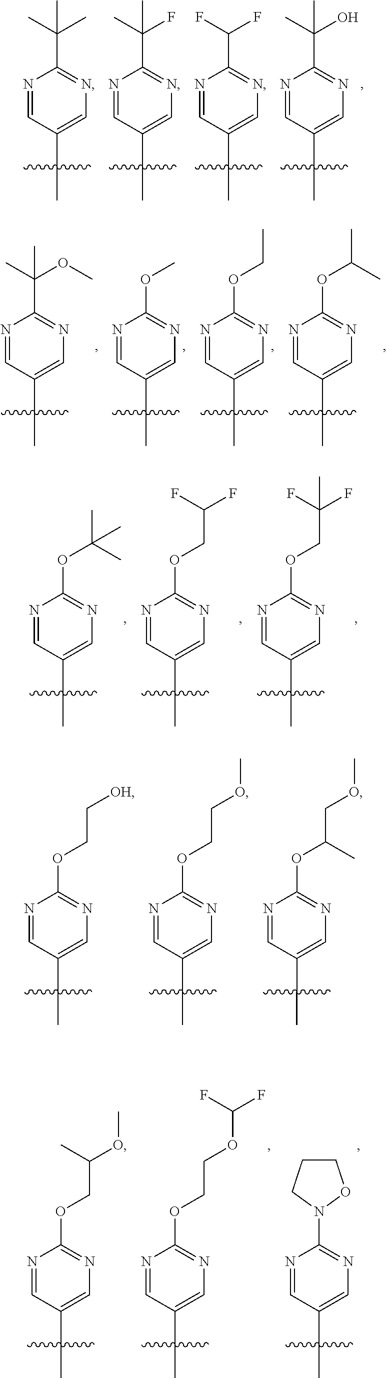 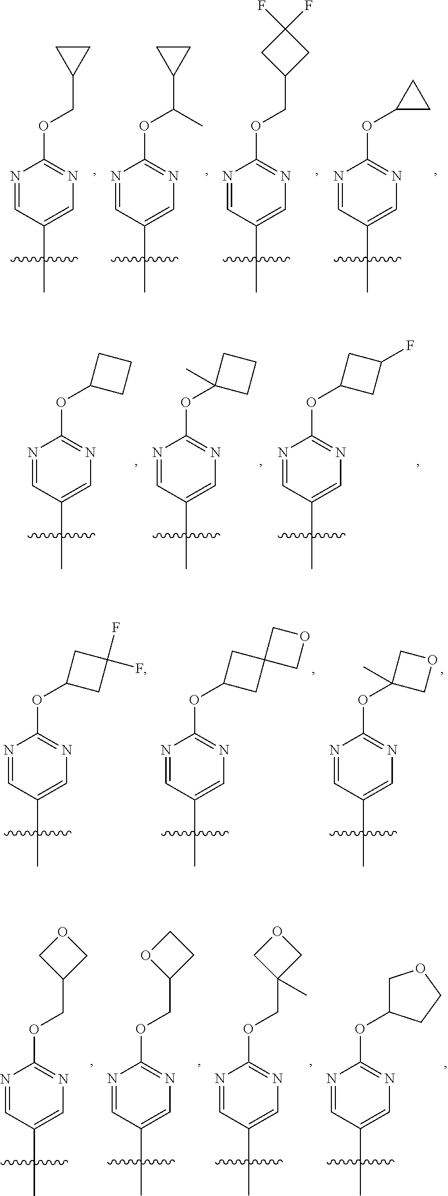 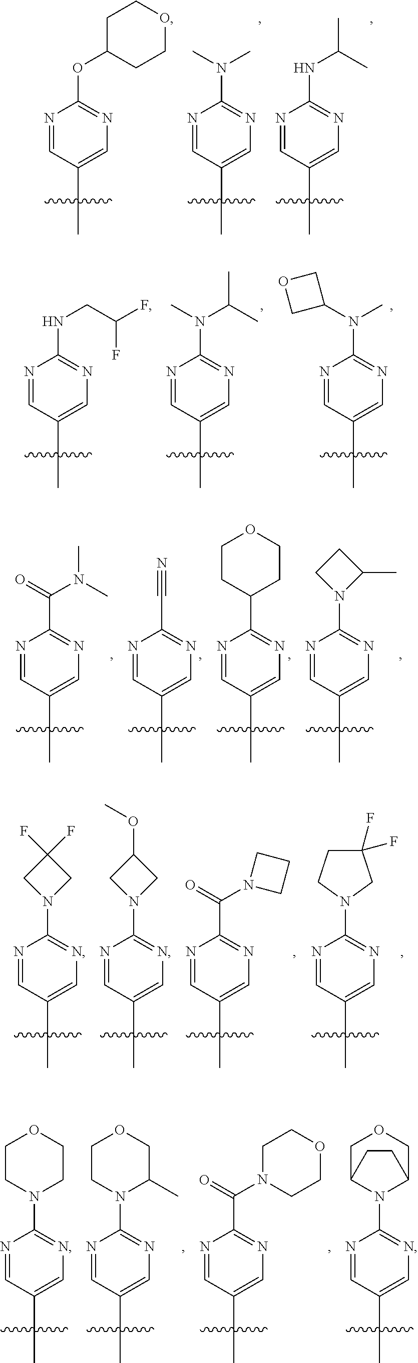 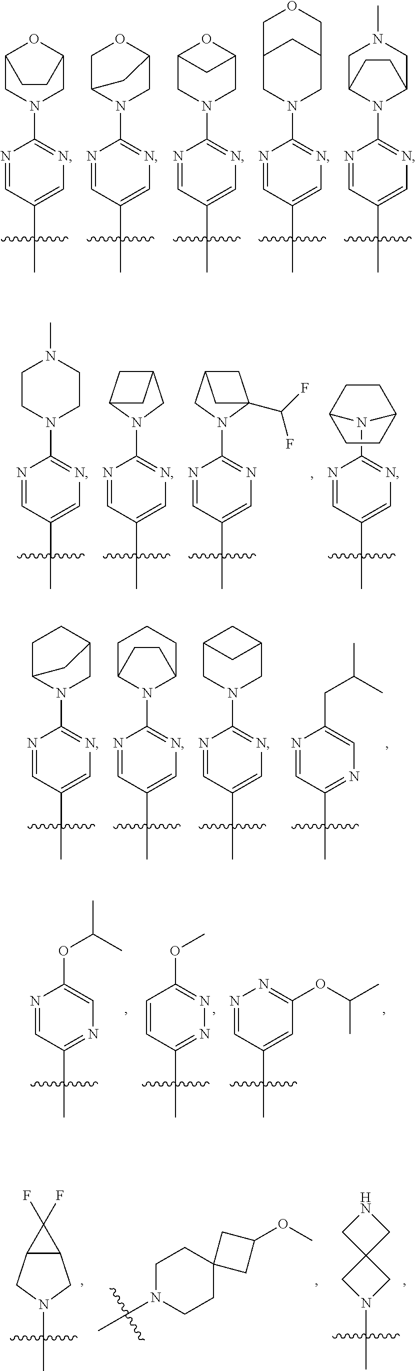 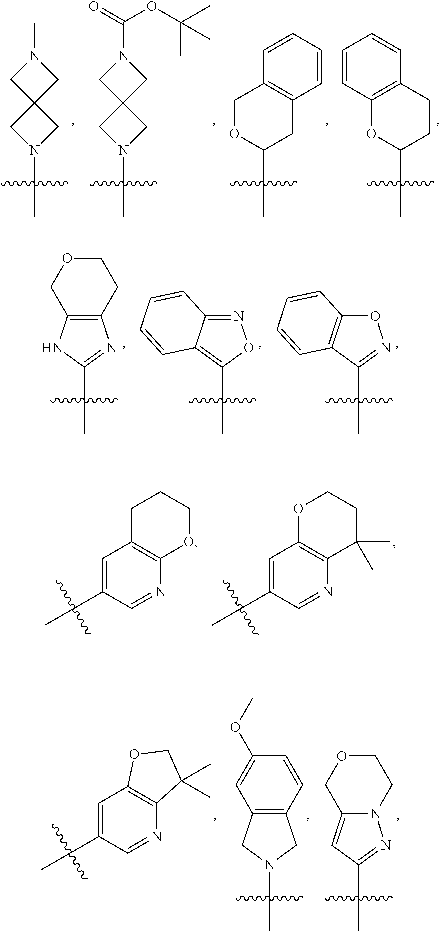
as a stereoisomer, enantiomer, or tautomer thereof or a mixture thereof; or a pharmaceutically acceptable salt, solvate, isotopologue, or prodrug thereof.

59. The compound of claim 1, wherein:
R³ has one of the following structures: 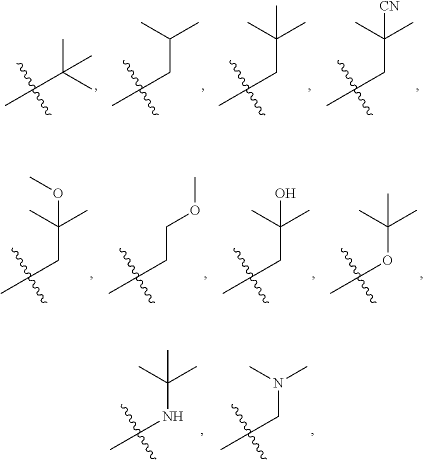
as a stereoisomer, enantiomer, or tautomer thereof or a mixture thereof, or a pharmaceutically acceptable salt, solvate, isotopologue, or prodrug thereof.

60. The compound of claim 1, wherein:
R³ has one of the following structures: 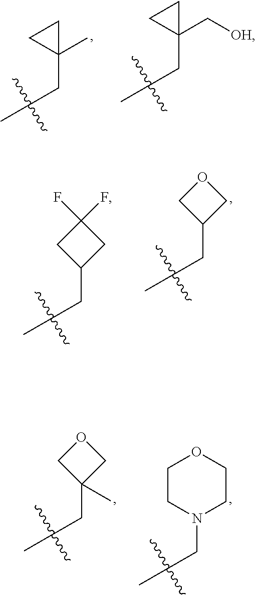
as a stereoisomer, enantiomer, or tautomer thereof or a mixture thereof; or a pharmaceutically acceptable salt, solvate, isotopologue, or prodrug thereof.

61. The compound of claim 1, wherein:
R³ has one of the following structures: 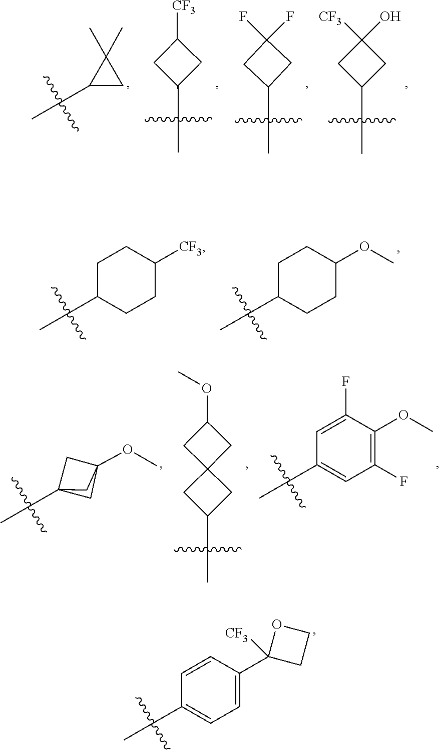
as a stereoisomer, enantiomer, or tautomer thereof or a mixture thereof, or a pharmaceutically acceptable salt, solvate, isotopologue, or prodrug thereof.

62. The compound of claim 1, wherein:
R³ has one of the following structures: 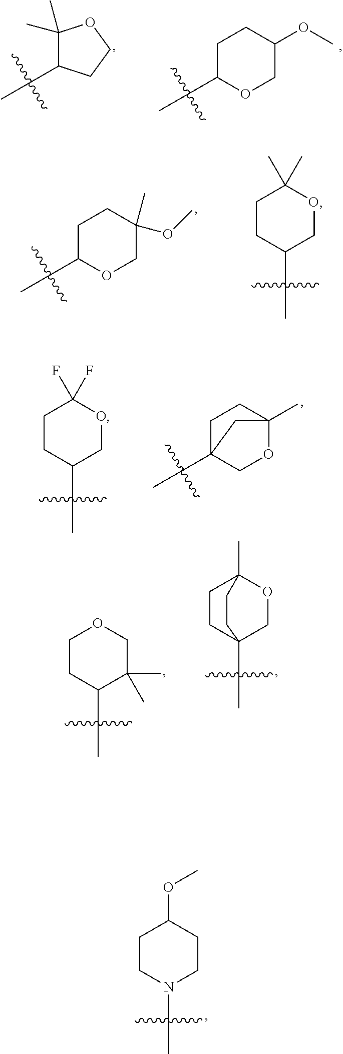
as a stereoisomer, enantiomer, or tautomer thereof or a mixture thereof, or a pharmaceutically acceptable salt, solvate, isotopologue, or prodrug thereof.

63. The compound of claim 1, wherein:
R³ has one of the following structures: 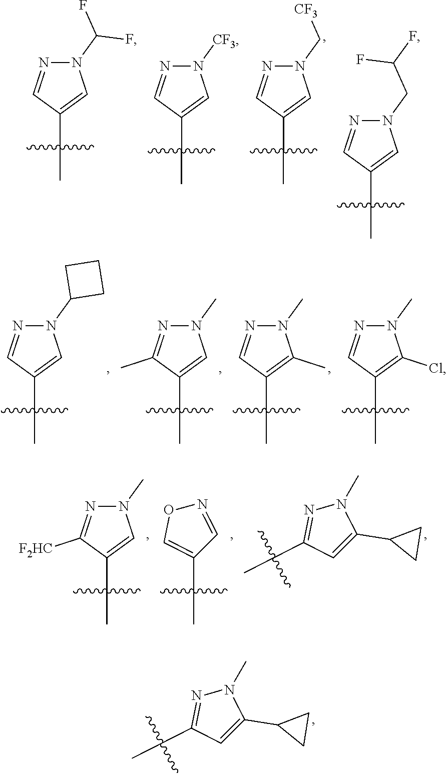 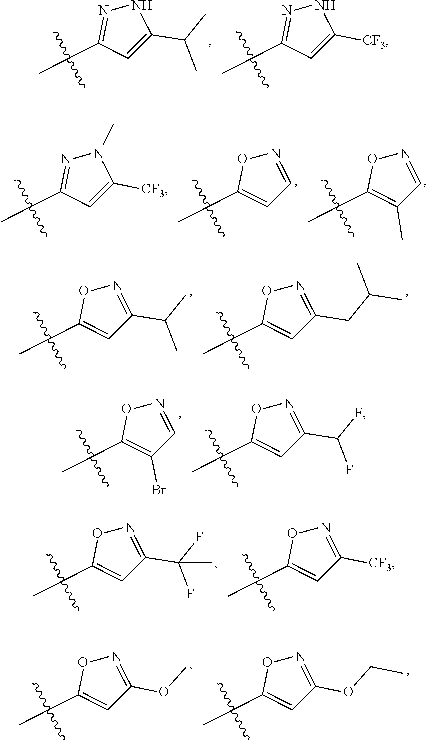 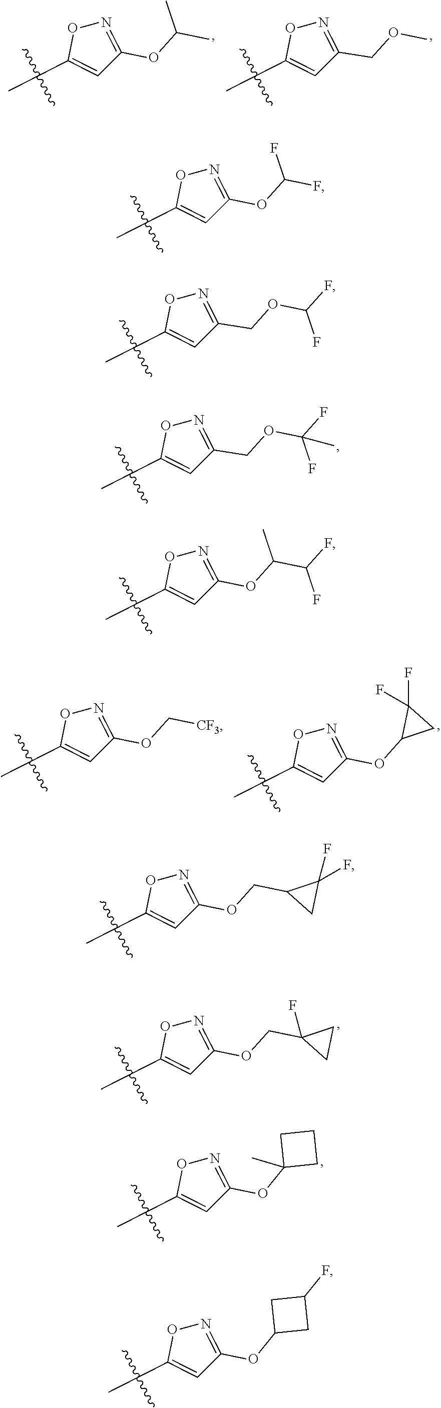 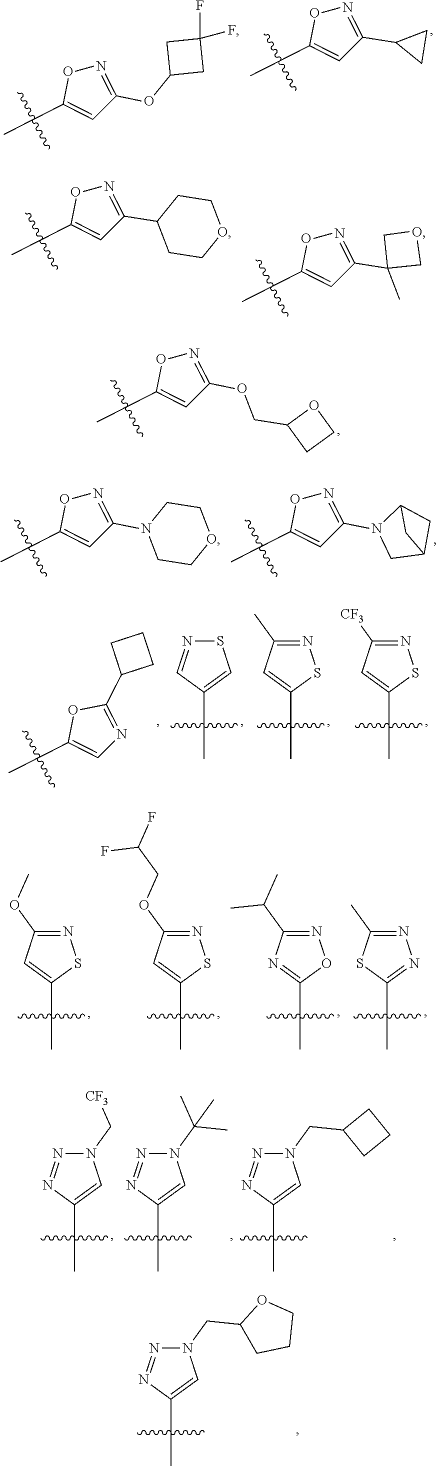 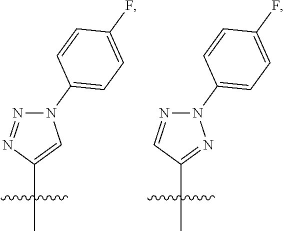 [SMILES: *c1cn(-c2ccc(F)cc2)nn1 ; *c1cnn(-c2ccc(F)cc2)n1]
as a stereoisomer, enantiomer, or tautomer thereof or a mixture thereof, or a pharmaceutically acceptable salt, solvate, isotopologue, or prodrug thereof.

64. The compound of claim 1, wherein:
R³ has one of the following structures: 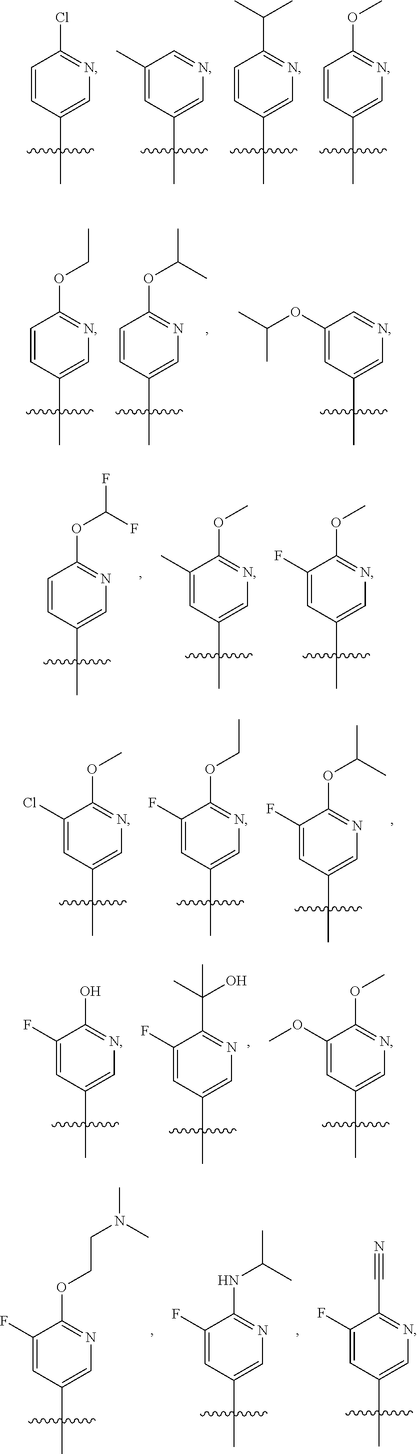 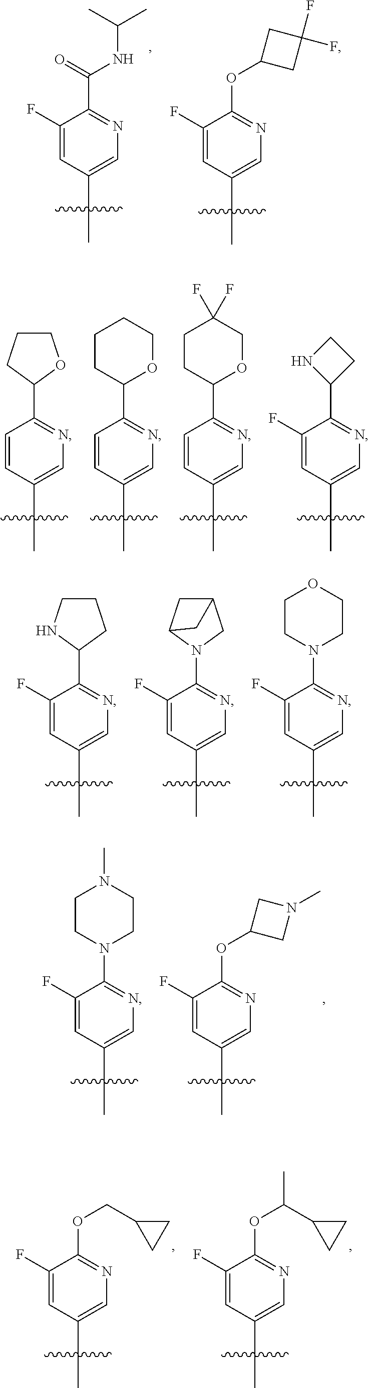 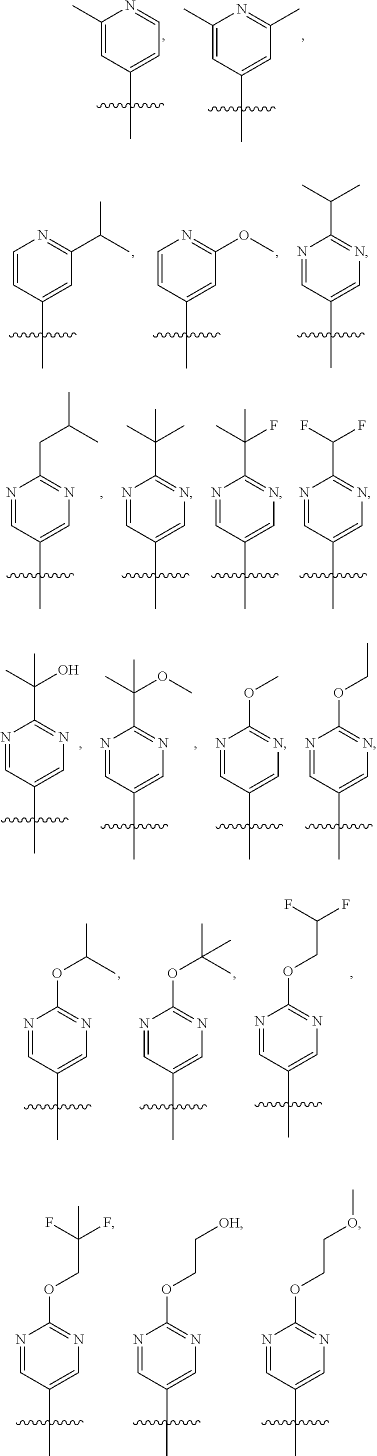 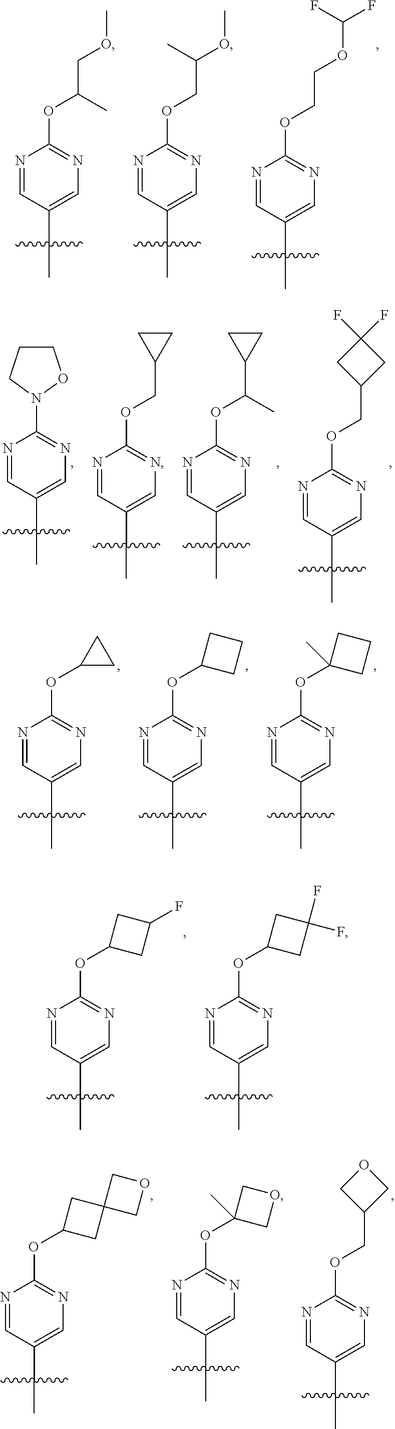 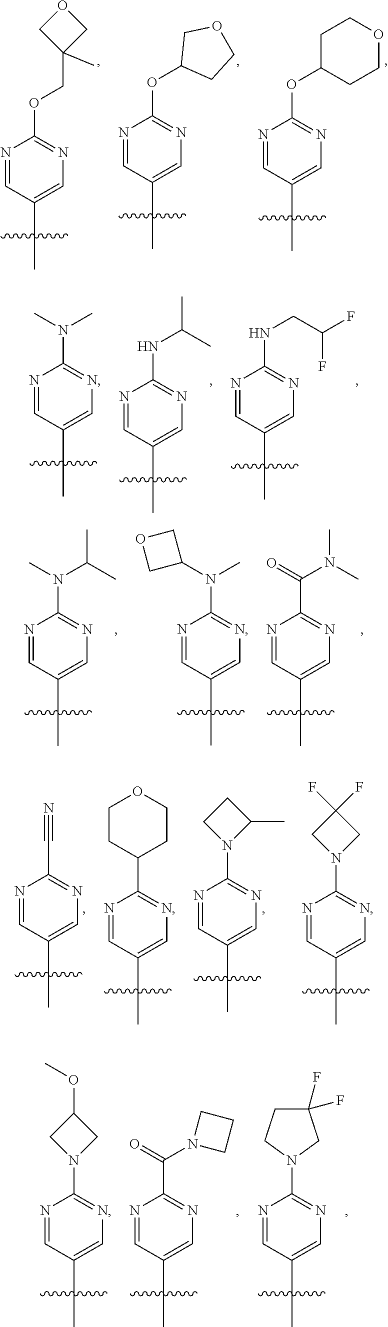 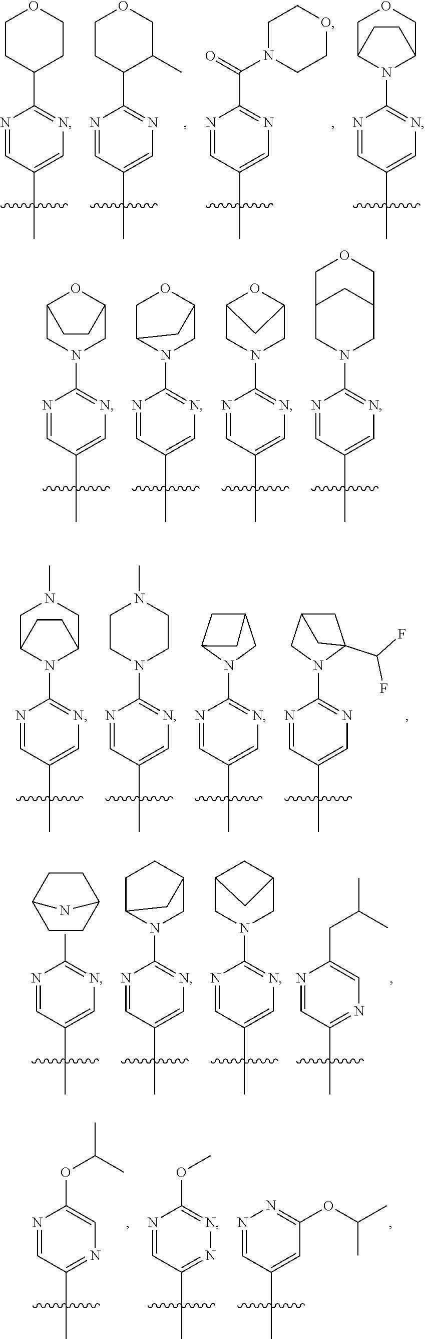
as a stereoisomer, enantiomer, or tautomer thereof or a mixture thereof, or a pharmaceutically acceptable salt, solvate, isotopologue, or prodrug thereof.

65. The compound of claim 1, wherein:
R³ has one of the following structures: 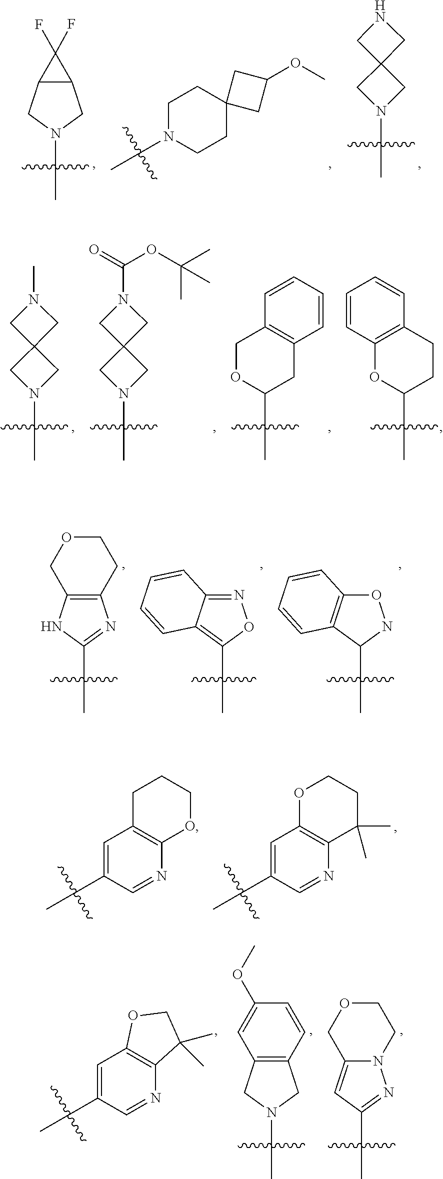
as a stereoisomer, enantiomer, or tautomer thereof or a mixture thereof, or a pharmaceutically acceptable salt, solvate, isotopologue, or prodrug thereof.

66. The compound of claim 1, wherein:
L is a direct bond;
as a stereoisomer, enantiomer, or tautomer thereof or a mixture thereof, or a pharmaceutically acceptable salt, solvate, isotopologue, or prodrug thereof.

67. The compound of claim 1, wherein:
L is —C(═O)NR⁴—;
as a stereoisomer, enantiomer, or tautomer thereof or a mixture thereof, or a pharmaceutically acceptable salt, solvate, isotopologue, or prodrug thereof.

68. The compound of claim 1, wherein:
L is —NR⁴C(═O)—;
as a stereoisomer, enantiomer, or tautomer thereof or a mixture thereof; or a pharmaceutically acceptable salt, solvate, isotopologue, or prodrug thereof.

69. The compound of claim 1, wherein:
R⁴ is hydrogen;
as a stereoisomer, enantiomer, or tautomer thereof or a mixture thereof, or a pharmaceutically acceptable salt, solvate, isotopologue, or prodrug thereof.

70. The compound of claim 1, wherein:
R⁴ is methyl;
as a stereoisomer, enantiomer, or tautomer thereof or a mixture thereof; or a pharmaceutically acceptable salt, solvate, isotopologue, or prodrug thereof.

71. A compound having one of the following structures: 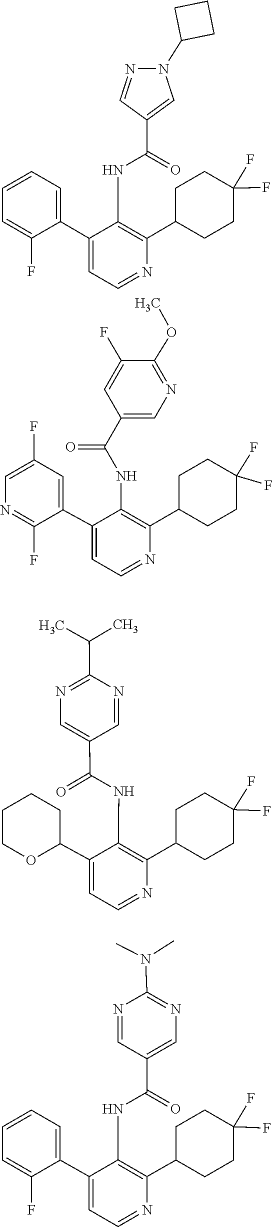 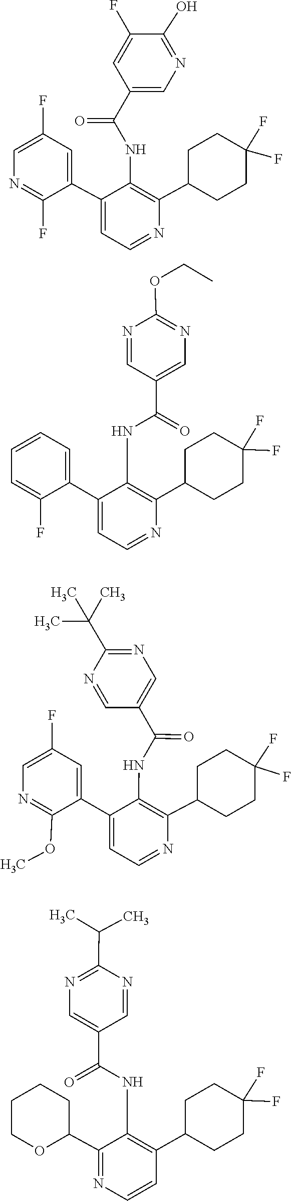 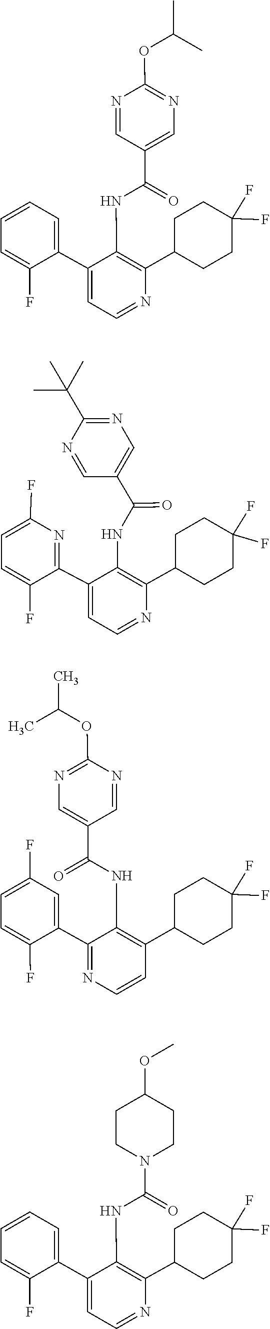 [SMILES: CC(C)Oc1ncc(C(=O)Nc2c(-c3ccccc3F)ccnc2C2CCC(F)(F)CC2)cn1 ; CC(C)(C)c1ncc(C(=O)Nc2c(-c3nc(F)ccc3F)ccnc2C2CCC(F)(F)CC2)cn1 ; CC(C)Oc1ncc(C(=O)Nc2c(C3CCC(F)(F)CC3)ccnc2-c2cc(F)ccc2F)cn1 ; COC1CCN(C(=O)Nc2c(-c3ccccc3F)ccnc2C2CCC(F)(F)CC2)CC1] 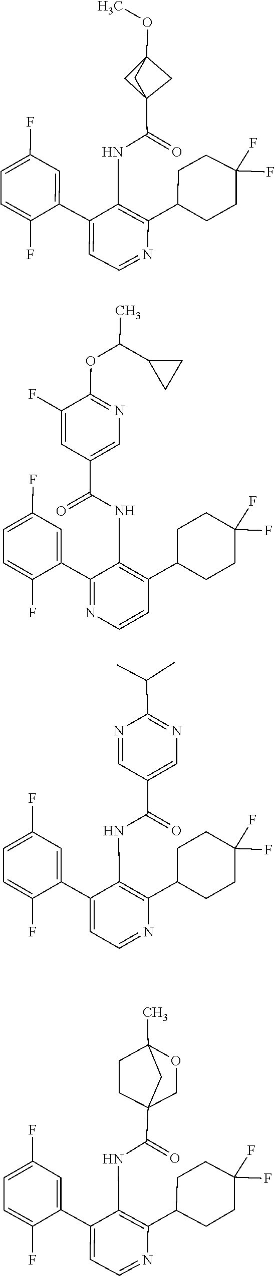 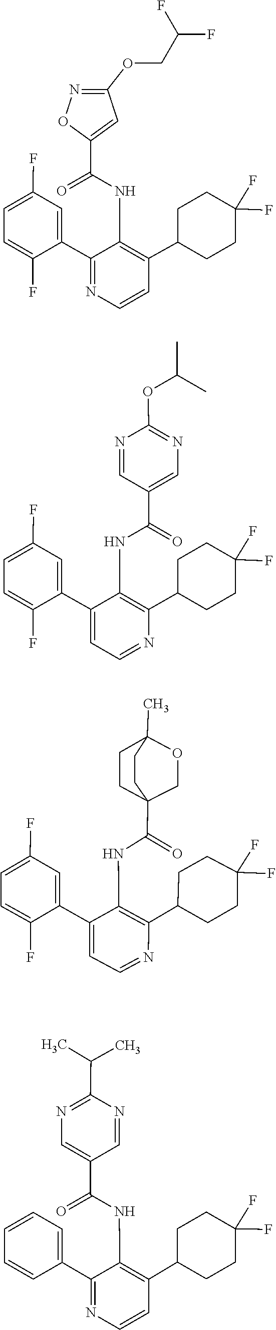 [SMILES: O=C(Nc1c(C2CCC(F)(F)CC2)ccnc1-c1cc(F)ccc1F)c1cc(OCC(F)F)no1 ; CC(C)Oc1ncc(C(=O)Nc2c(-c3cc(F)ccc3F)ccnc2C2CCC(F)(F)CC2)cn1 ; CC12CCC(C(=O)Nc3c(-c4cc(F)ccc4F)ccnc3C3CCC(F)(F)CC3)(CC1)CO2 ; CC(C)c1ncc(C(=O)Nc2c(C3CCC(F)(F)CC3)ccnc2-c2ccccc2)cn1] 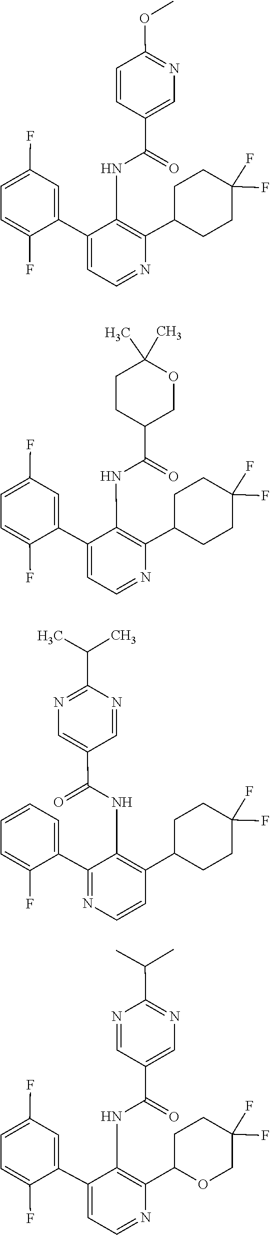 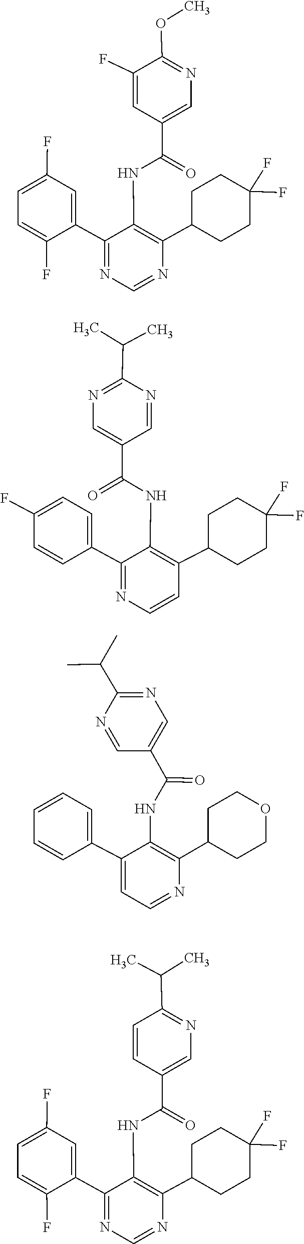 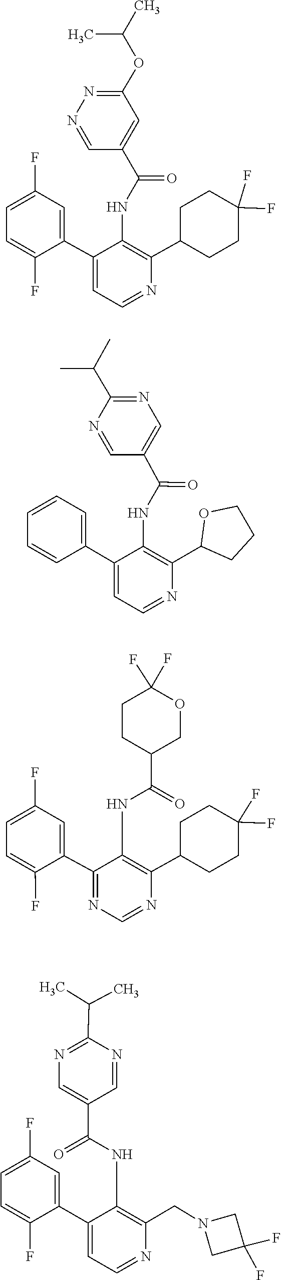 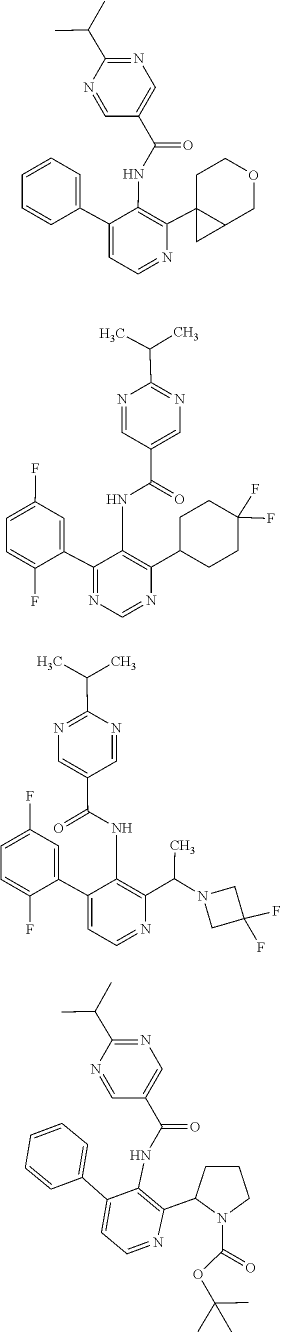 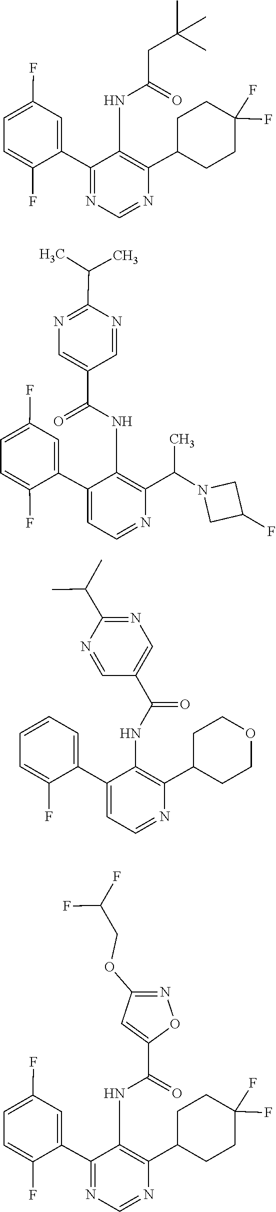 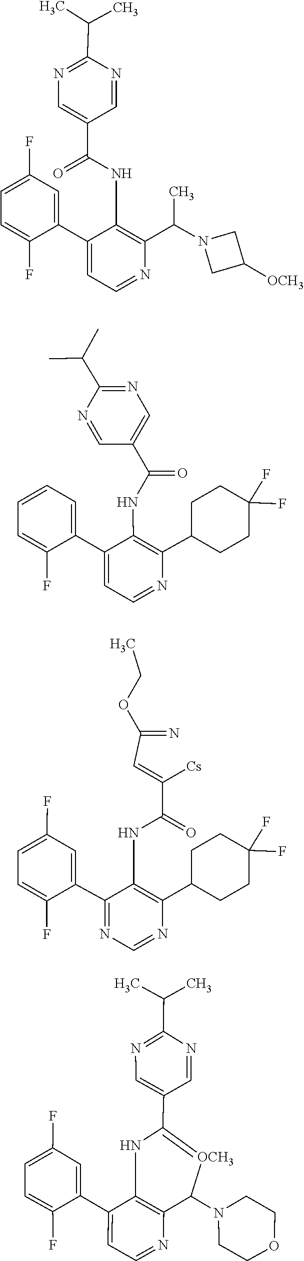 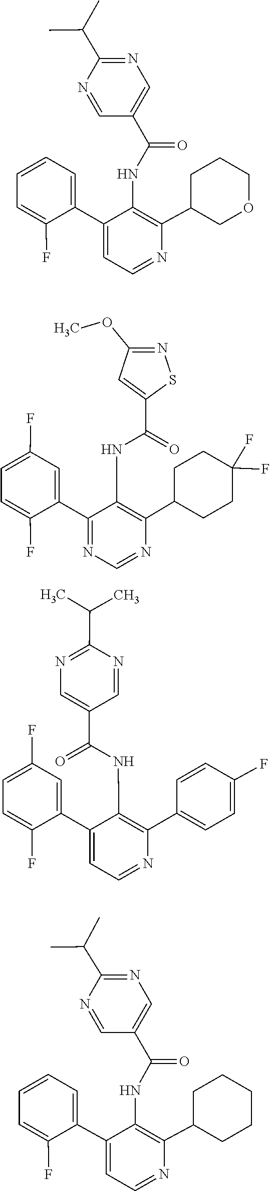 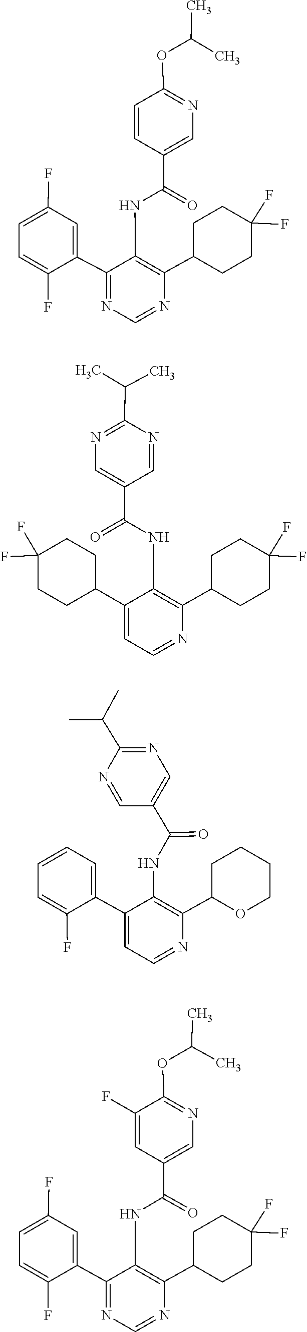 [SMILES: CC(C)Oc1ccc(C(=O)Nc2c(-c3cc(F)ccc3F)ncnc2C2CCC(F)(F)CC2)cn1 ; CC(C)c1ncc(C(=O)Nc2c(C3CCC(F)(F)CC3)ccnc2C2CCC(F)(F)CC2)cn1 ; CC(C)c1ncc(C(=O)Nc2c(-c3ccccc3F)ccnc2C2CCCCO2)cn1 ; CC(C)Oc1ncc(C(=O)Nc2c(-c3cc(F)ccc3F)ncnc2C2CCC(F)(F)CC2)cc1F] 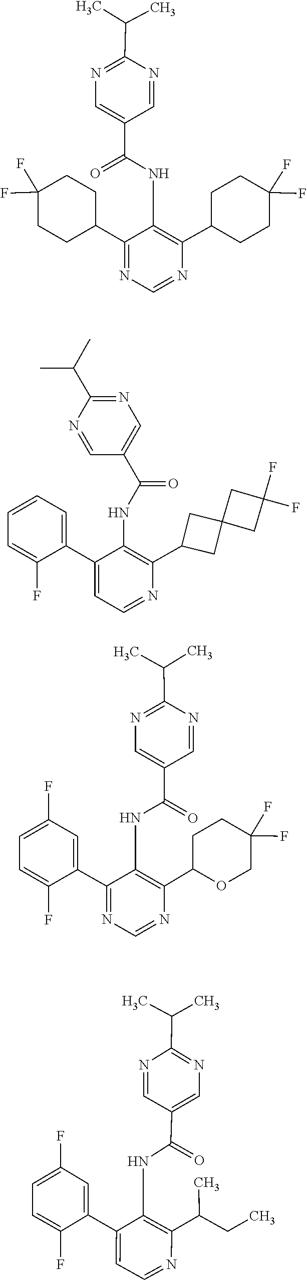 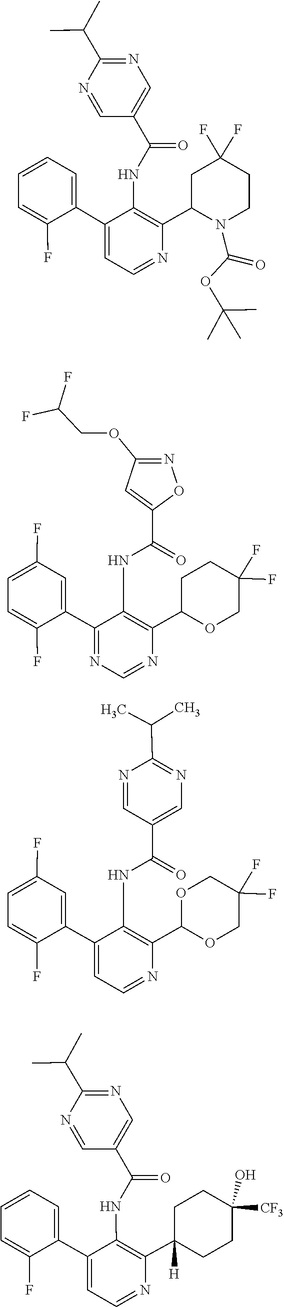 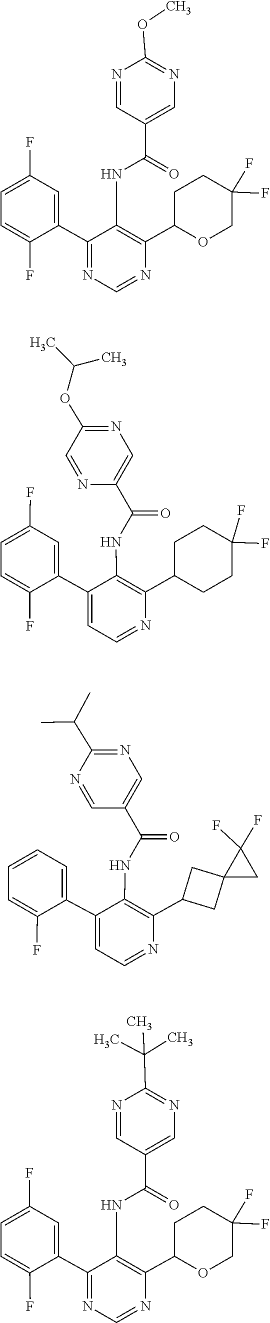 [SMILES: COc1ncc(C(=O)Nc2c(-c3cc(F)ccc3F)ncnc2C2CCC(F)(F)CO2)cn1 ; CC(C)Oc1cnc(C(=O)Nc2c(-c3cc(F)ccc3F)ccnc2C2CCC(F)(F)CC2)cn1 ; CC(C)c1ncc(C(=O)Nc2c(-c3ccccc3F)ccnc2C2CC3(C2)CC3(F)F)cn1 ; CC(C)(C)c1ncc(C(=O)Nc2c(-c3cc(F)ccc3F)ncnc2C2CCC(F)(F)CO2)cn1] 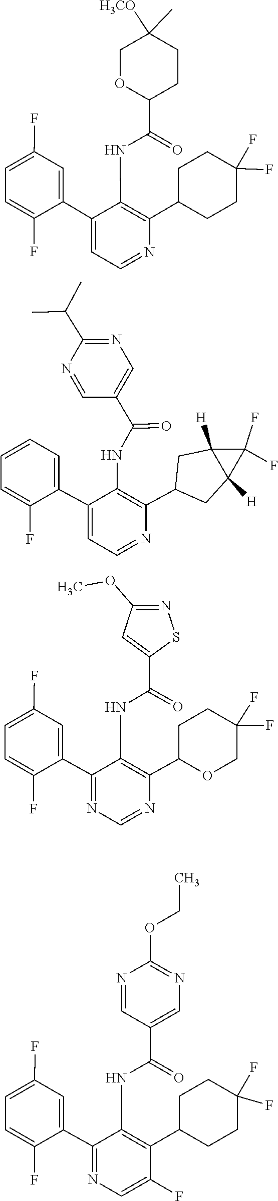 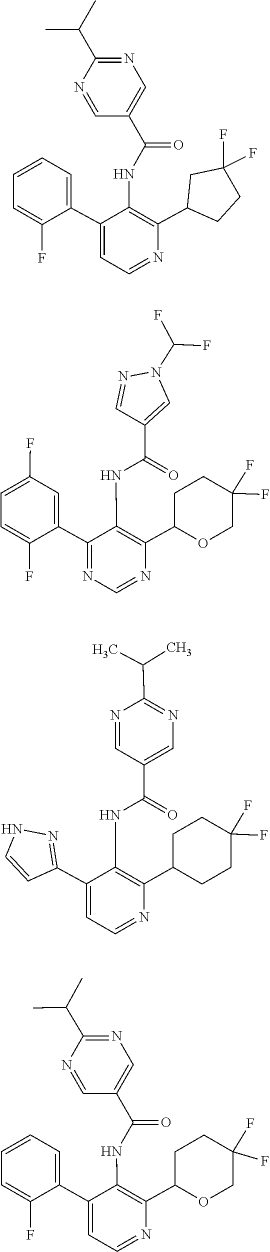 [SMILES: CC(C)c1ncc(C(=O)Nc2c(-c3ccccc3F)ccnc2C2CCC(F)(F)C2)cn1 ; O=C(Nc1c(-c2cc(F)ccc2F)ncnc1C1CCC(F)(F)CO1)c1cnn(C(F)F)c1 ; CC(C)c1ncc(C(=O)Nc2c(-c3cc[nH]n3)ccnc2C2CCC(F)(F)CC2)cn1 ; CC(C)c1ncc(C(=O)Nc2c(-c3ccccc3F)ccnc2C2CCC(F)(F)CO2)cn1] 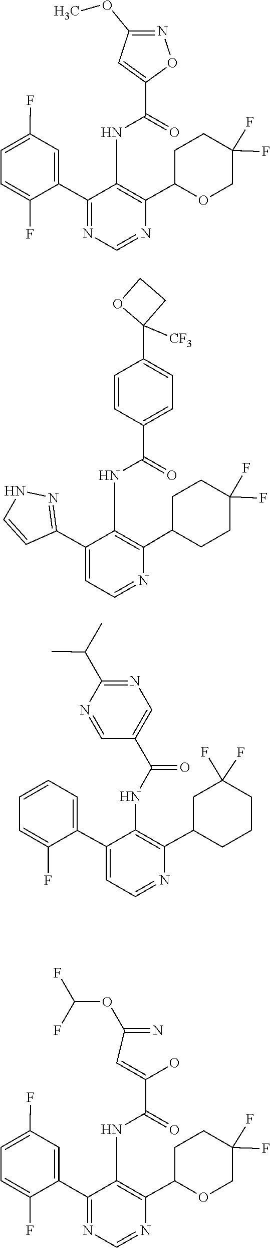 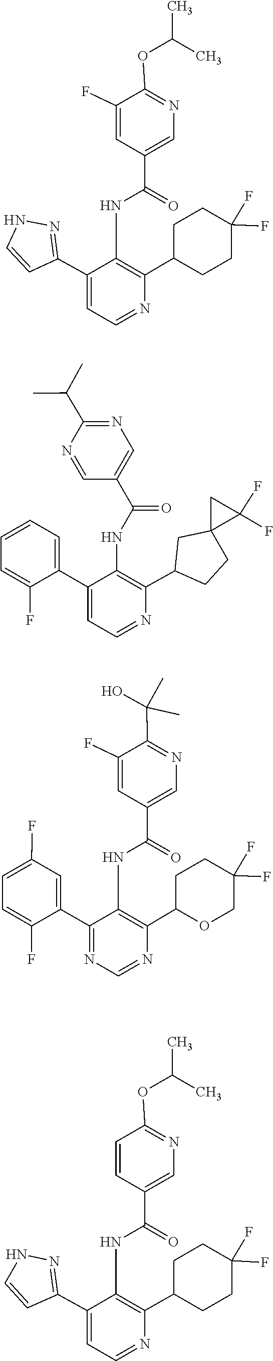 [SMILES: CC(C)Oc1ncc(C(=O)Nc2c(-c3cc[nH]n3)ccnc2C2CCC(F)(F)CC2)cc1F ; CC(C)c1ncc(C(=O)Nc2c(-c3ccccc3F)ccnc2C2CCC3(C2)CC3(F)F)cn1 ; CC(C)(O)c1ncc(C(=O)Nc2c(-c3cc(F)ccc3F)ncnc2C2CCC(F)(F)CO2)cc1F ; CC(C)Oc1ccc(C(=O)Nc2c(-c3cc[nH]n3)ccnc2C2CCC(F)(F)CC2)cn1] 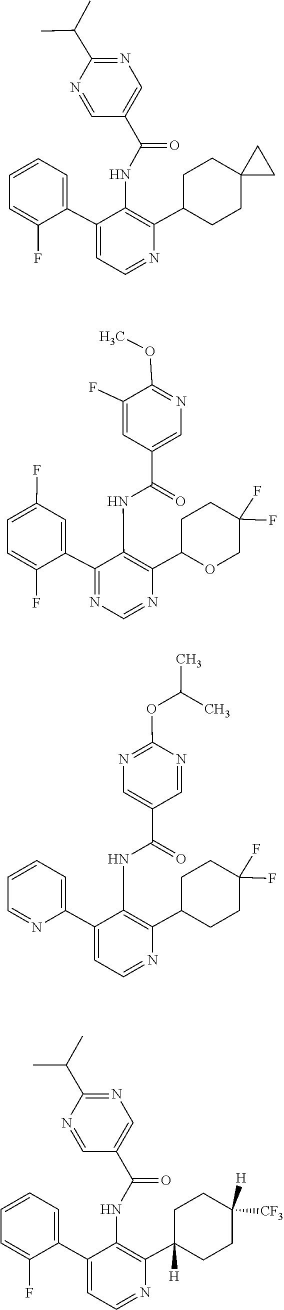 [SMILES: CC(C)c1ncc(C(=O)Nc2c(-c3ccccc3F)ccnc2C2CCC3(CC2)CC3)cn1 ; COc1ncc(C(=O)Nc2c(-c3cc(F)ccc3F)ncnc2C2CCC(F)(F)CO2)cc1F ; CC(C)Oc1ncc(C(=O)Nc2c(-c3ccccn3)ccnc2C2CCC(F)(F)CC2)cn1 ; CC(C)c1ncc(C(=O)Nc2c(-c3ccccc3F)ccnc2[C@H]2CC[C@@H](C(F)(F)F)CC2)cn1] 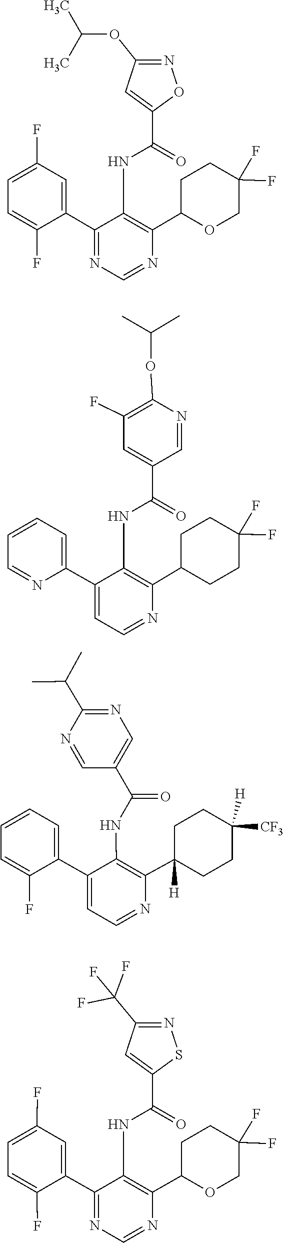 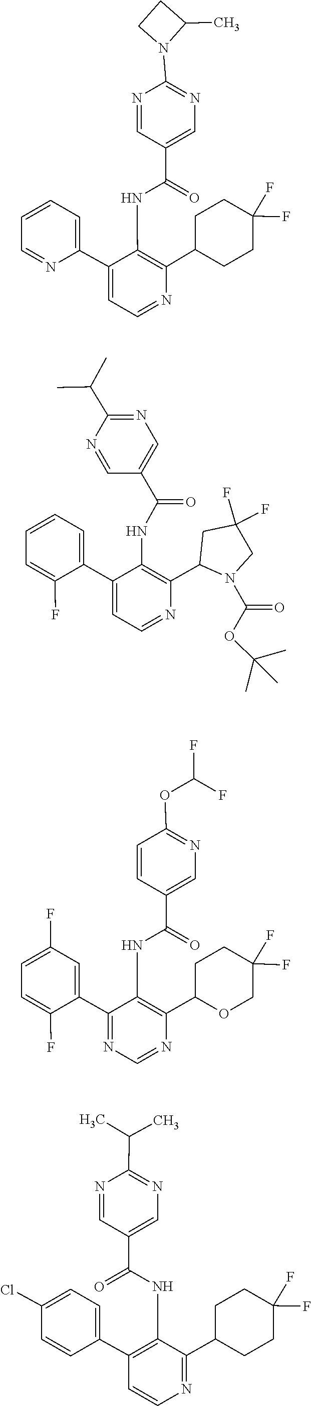 [SMILES: CC1CCN1c1ncc(C(=O)Nc2c(-c3ccccn3)ccnc2C2CCC(F)(F)CC2)cn1 ; CC(C)c1ncc(C(=O)Nc2c(-c3ccccc3F)ccnc2C2CC(F)(F)CN2C(=O)OC(C)(C)C)cn1 ; O=C(Nc1c(-c2cc(F)ccc2F)ncnc1C1CCC(F)(F)CO1)c1ccc(OC(F)F)nc1 ; CC(C)c1ncc(C(=O)Nc2c(-c3ccc(Cl)cc3)ccnc2C2CCC(F)(F)CC2)cn1] 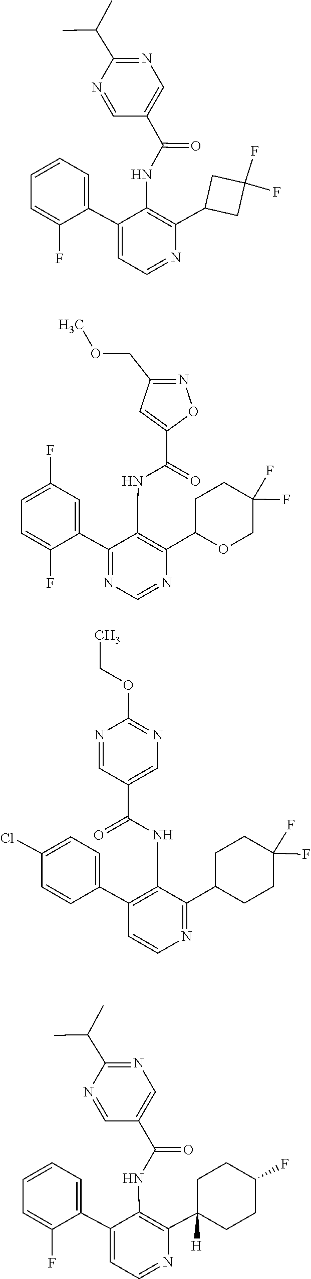 [SMILES: CC(C)c1ncc(C(=O)Nc2c(-c3ccccc3F)ccnc2C2CC(F)(F)C2)cn1 ; COCc1cc(C(=O)Nc2c(-c3cc(F)ccc3F)ncnc2C2CCC(F)(F)CO2)on1 ; CCOc1ncc(C(=O)Nc2c(-c3ccc(Cl)cc3)ccnc2C2CCC(F)(F)CC2)cn1 ; CC(C)c1ncc(C(=O)Nc2c(-c3ccccc3F)ccnc2[C@H]2CC[C@@H](F)CC2)cn1] 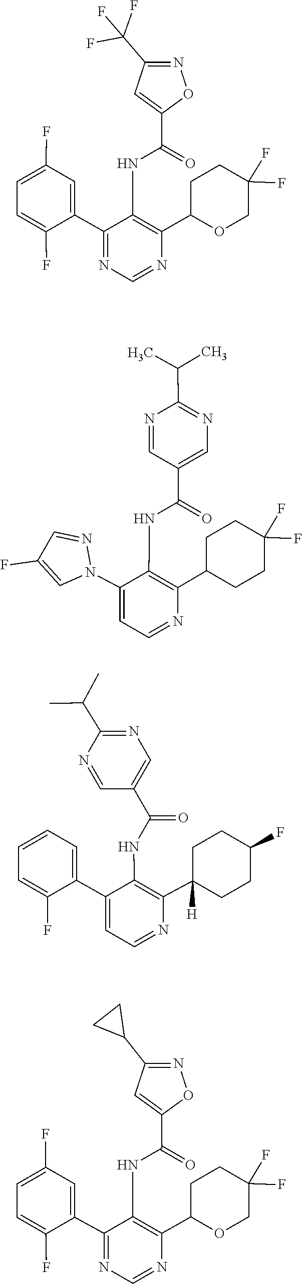 [SMILES: O=C(Nc1c(-c2cc(F)ccc2F)ncnc1C1CCC(F)(F)CO1)c1cc(C(F)(F)F)no1 ; CC(C)c1ncc(C(=O)Nc2c(-n3cc(F)cn3)ccnc2C2CCC(F)(F)CC2)cn1 ; CC(C)c1ncc(C(=O)Nc2c(-c3ccccc3F)ccnc2[C@H]2CC[C@H](F)CC2)cn1 ; O=C(Nc1c(-c2cc(F)ccc2F)ncnc1C1CCC(F)(F)CO1)c1cc(C2CC2)no1] 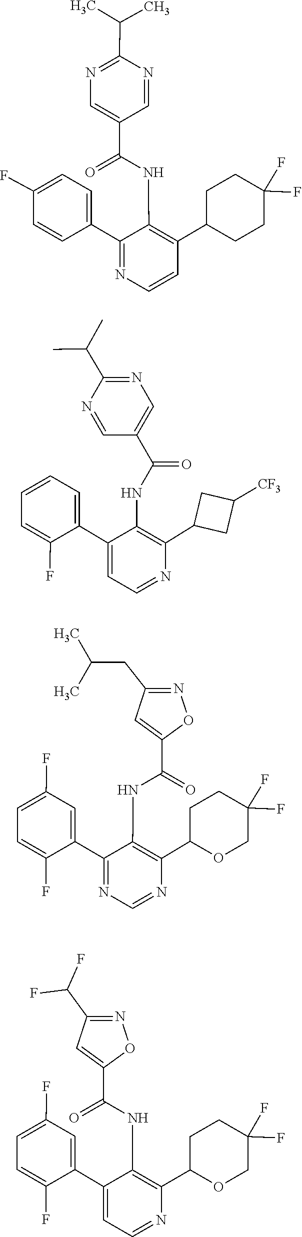 [SMILES: CC(C)c1ncc(C(=O)Nc2c(C3CCC(F)(F)CC3)ccnc2-c2ccc(F)cc2)cn1 ; CC(C)c1ncc(C(=O)Nc2c(-c3ccccc3F)ccnc2C2CC(C(F)(F)F)C2)cn1 ; CC(C)Cc1cc(C(=O)Nc2c(-c3cc(F)ccc3F)ncnc2C2CCC(F)(F)CO2)on1 ; O=C(Nc1c(-c2cc(F)ccc2F)ccnc1C1CCC(F)(F)CO1)c1cc(C(F)F)no1] 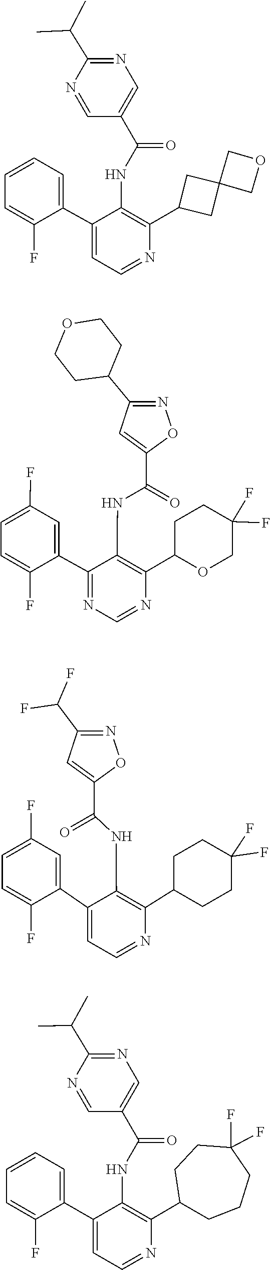 [SMILES: CC(C)c1ncc(C(=O)Nc2c(-c3ccccc3F)ccnc2C2CC3(COC3)C2)cn1 ; O=C(Nc1c(-c2cc(F)ccc2F)ncnc1C1CCC(F)(F)CO1)c1cc(C2CCOCC2)no1 ; O=C(Nc1c(-c2cc(F)ccc2F)ccnc1C1CCC(F)(F)CC1)c1cc(C(F)F)no1 ; CC(C)c1ncc(C(=O)Nc2c(-c3ccccc3F)ccnc2C2CCCC(F)(F)CC2)cn1] 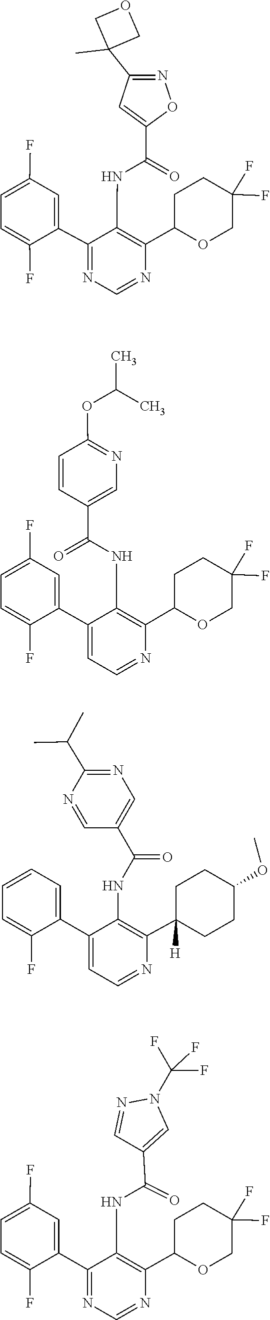 [SMILES: CC1(c2cc(C(=O)Nc3c(-c4cc(F)ccc4F)ncnc3C3CCC(F)(F)CO3)on2)COC1 ; CC(C)Oc1ccc(C(=O)Nc2c(-c3cc(F)ccc3F)ccnc2C2CCC(F)(F)CO2)cn1 ; CO[C@H]1CC[C@@H](c2nccc(-c3ccccc3F)c2NC(=O)c2cnc(C(C)C)nc2)CC1 ; O=C(Nc1c(-c2cc(F)ccc2F)ncnc1C1CCC(F)(F)CO1)c1cnn(C(F)(F)F)c1] 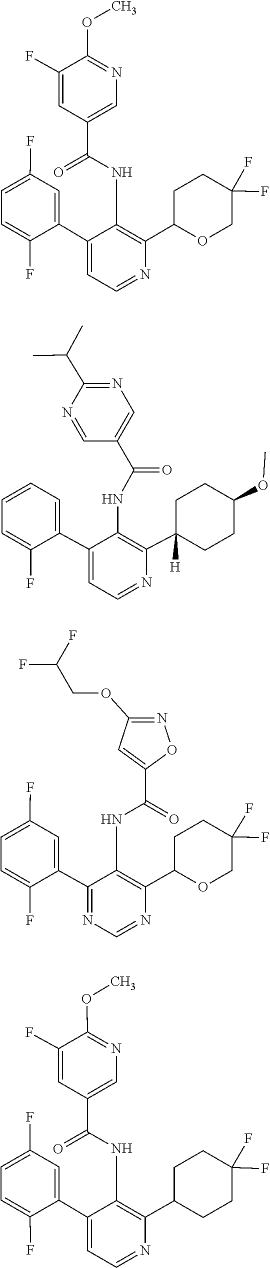 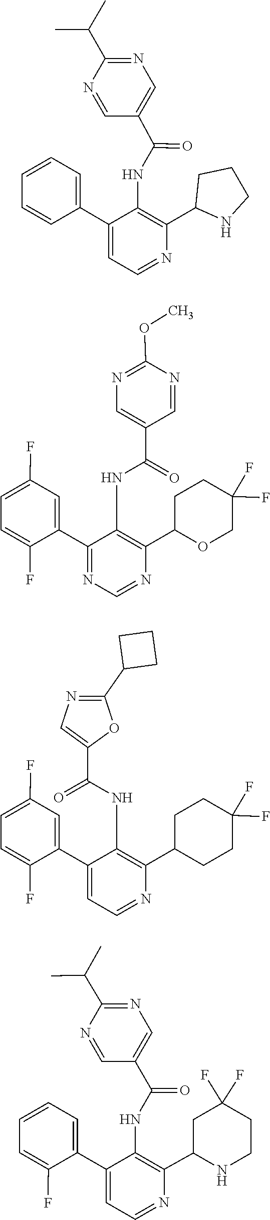 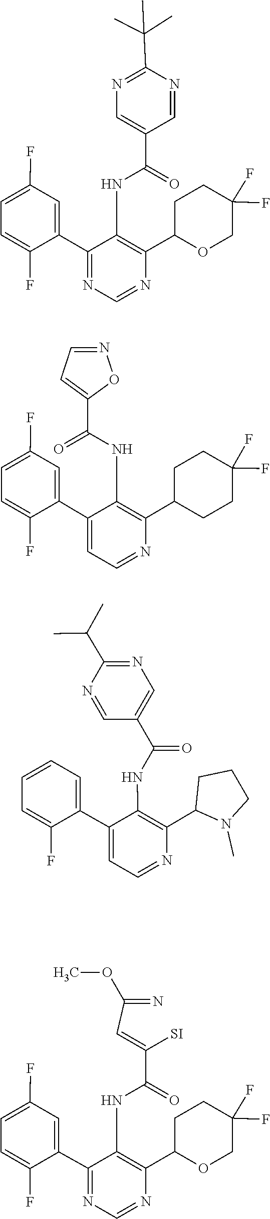 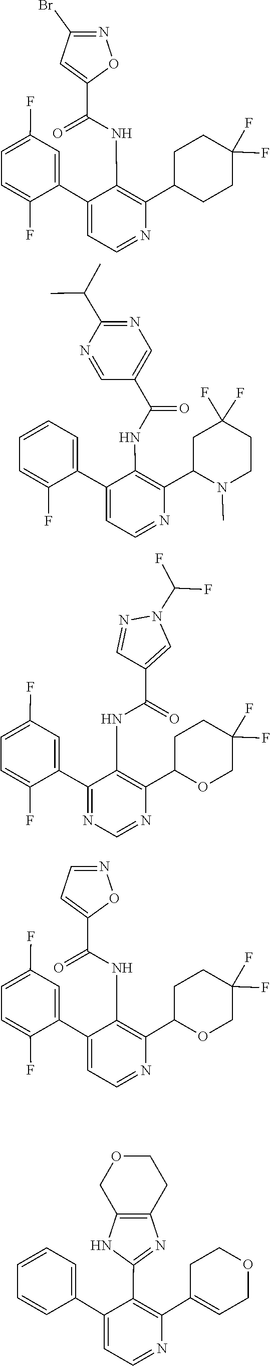 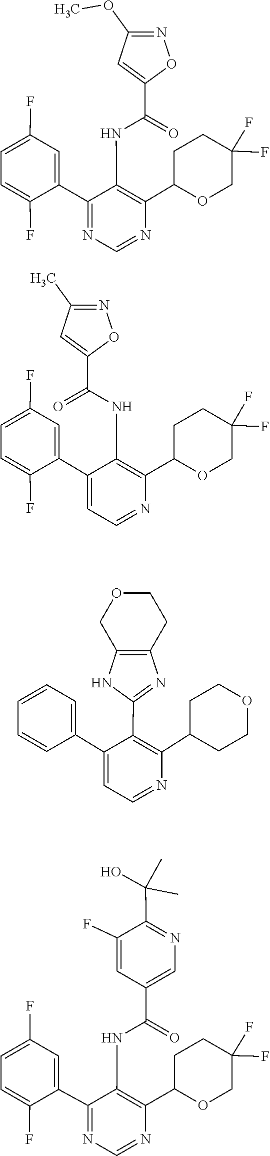 [SMILES: COc1cc(C(=O)Nc2c(-c3cc(F)ccc3F)ncnc2C2CCC(F)(F)CO2)on1 ; Cc1cc(C(=O)Nc2c(-c3cc(F)ccc3F)ccnc2C2CCC(F)(F)CO2)on1 ; c1ccc(-c2ccnc(C3CCOCC3)c2-c2nc3c([nH]2)COCC3)cc1 ; CC(C)(O)c1ncc(C(=O)Nc2c(-c3cc(F)ccc3F)ncnc2C2CCC(F)(F)CO2)cc1F] 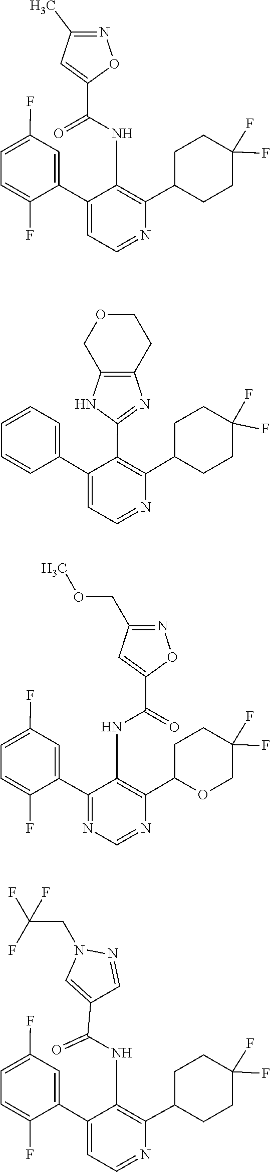 [SMILES: Cc1cc(C(=O)Nc2c(-c3cc(F)ccc3F)ccnc2C2CCC(F)(F)CC2)on1 ; FC1(F)CCC(c2nccc(-c3ccccc3)c2-c2nc3c([nH]2)COCC3)CC1 ; COCc1cc(C(=O)Nc2c(-c3cc(F)ccc3F)ncnc2C2CCC(F)(F)CO2)on1 ; O=C(Nc1c(-c2cc(F)ccc2F)ccnc1C1CCC(F)(F)CC1)c1cnn(CC(F)(F)F)c1] 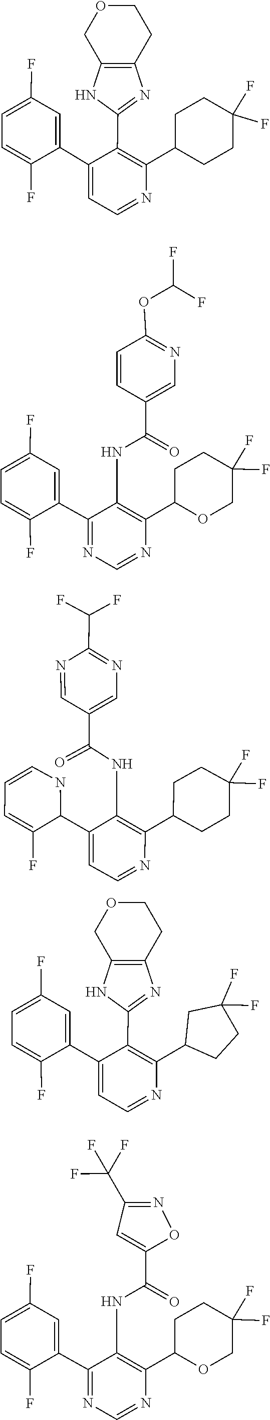 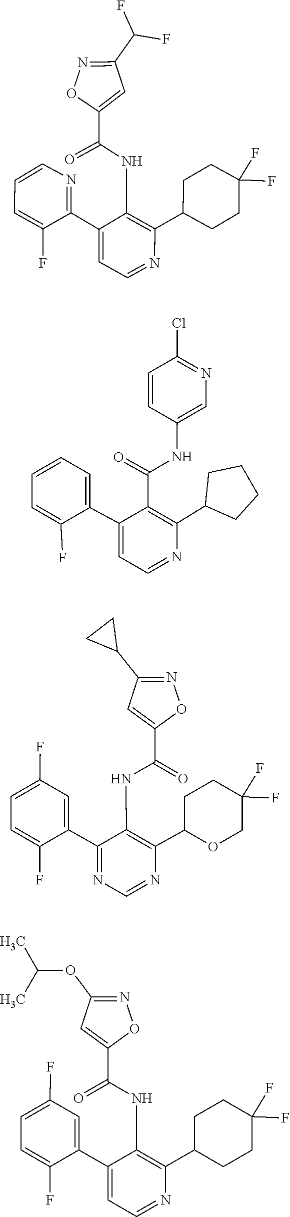 [SMILES: O=C(Nc1c(-c2ncccc2F)ccnc1C1CCC(F)(F)CC1)c1cc(C(F)F)no1 ; O=C(Nc1ccc(Cl)nc1)c1c(-c2ccccc2F)ccnc1C1CCCC1 ; O=C(Nc1c(-c2cc(F)ccc2F)ncnc1C1CCC(F)(F)CO1)c1cc(C2CC2)no1 ; CC(C)Oc1cc(C(=O)Nc2c(-c3cc(F)ccc3F)ccnc2C2CCC(F)(F)CC2)on1] 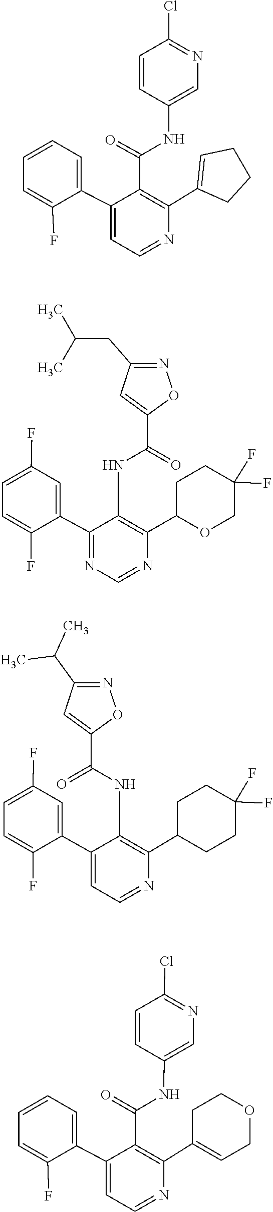 [SMILES: O=C(Nc1ccc(Cl)nc1)c1c(-c2ccccc2F)ccnc1C1=CCCC1 ; CC(C)Cc1cc(C(=O)Nc2c(-c3cc(F)ccc3F)ncnc2C2CCC(F)(F)CO2)on1 ; CC(C)c1cc(C(=O)Nc2c(-c3cc(F)ccc3F)ccnc2C2CCC(F)(F)CC2)on1 ; O=C(Nc1ccc(Cl)nc1)c1c(-c2ccccc2F)ccnc1C1=CCOCC1] 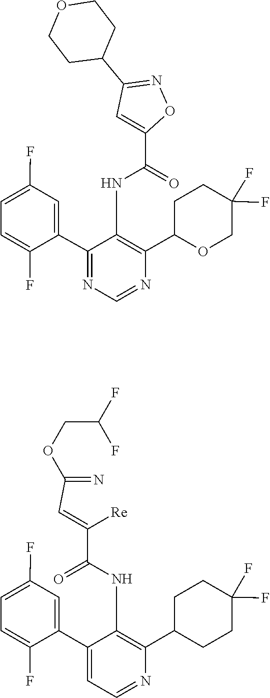 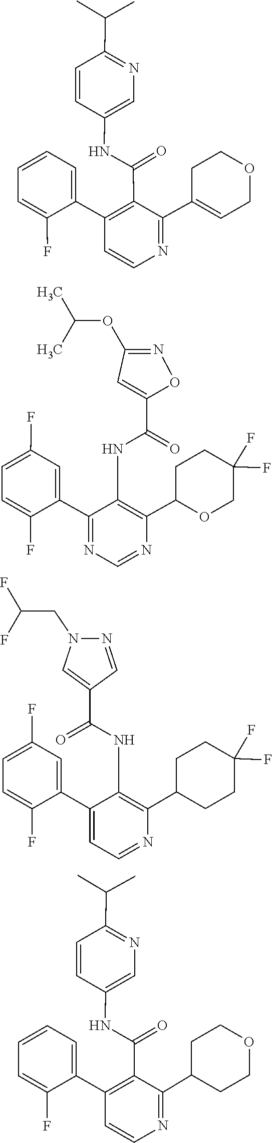 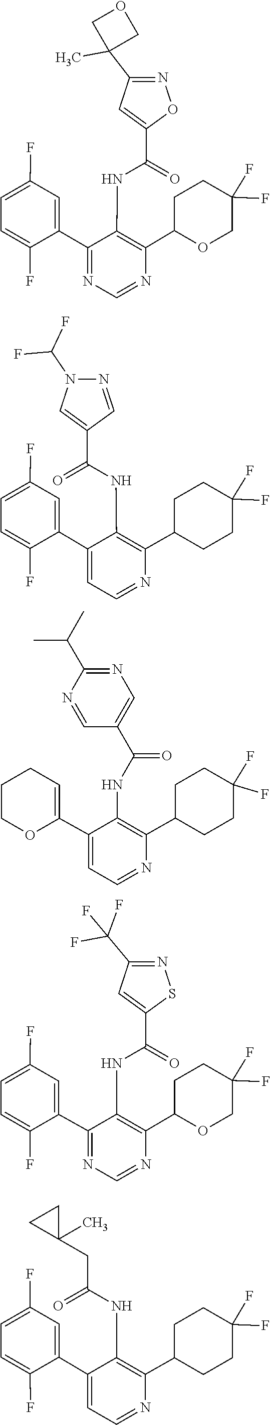 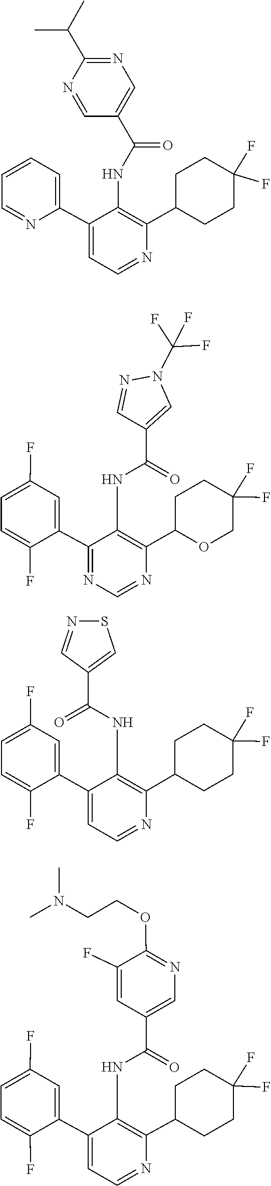 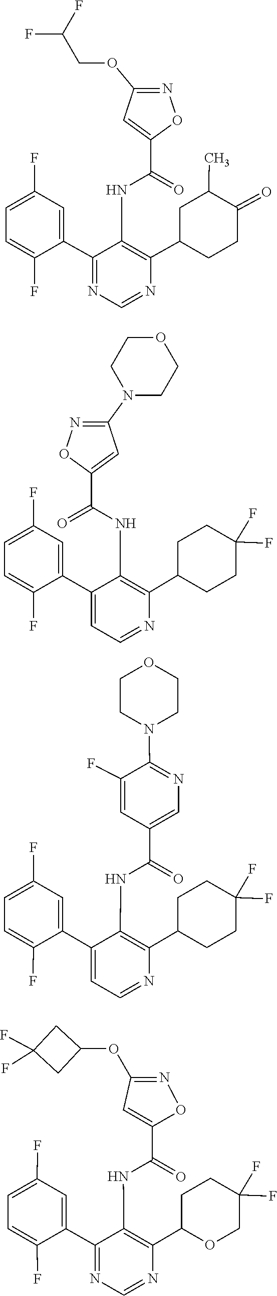 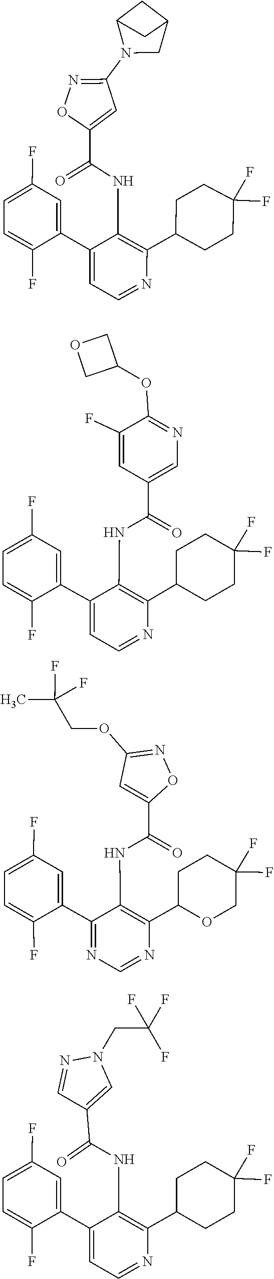 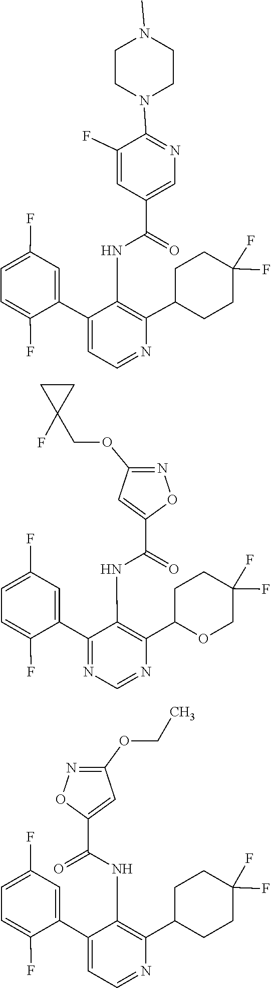 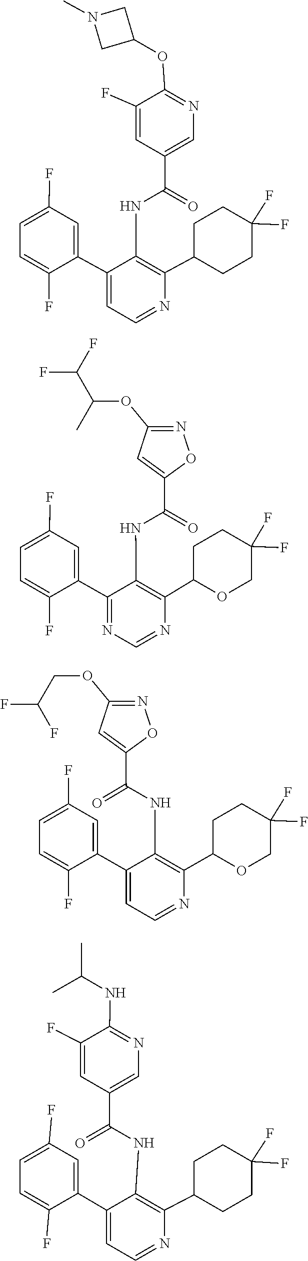 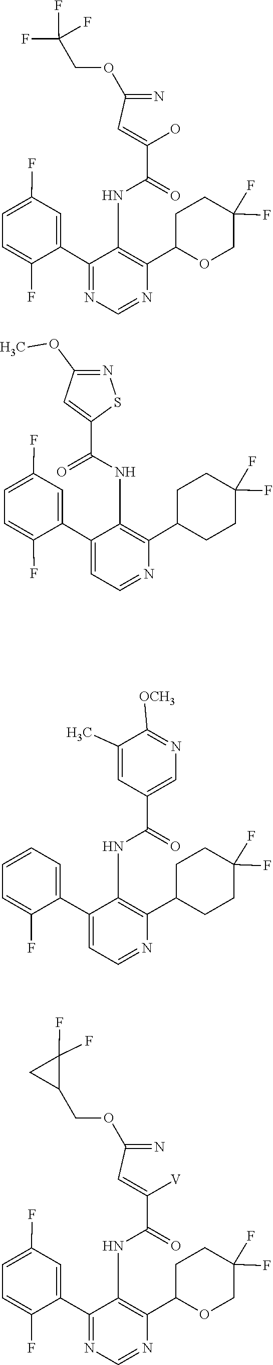 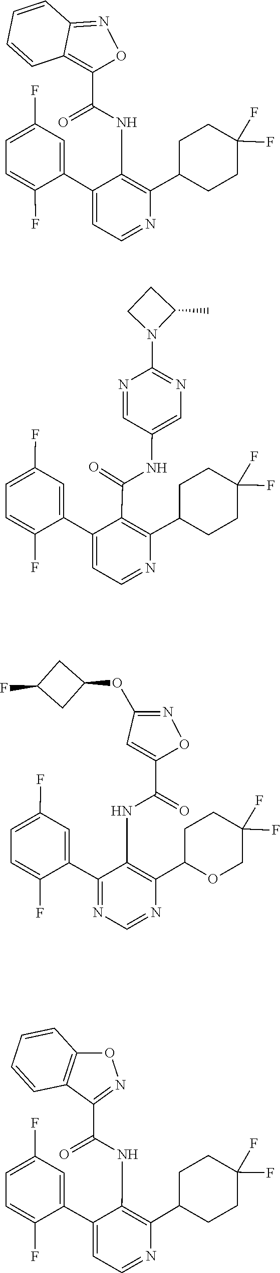 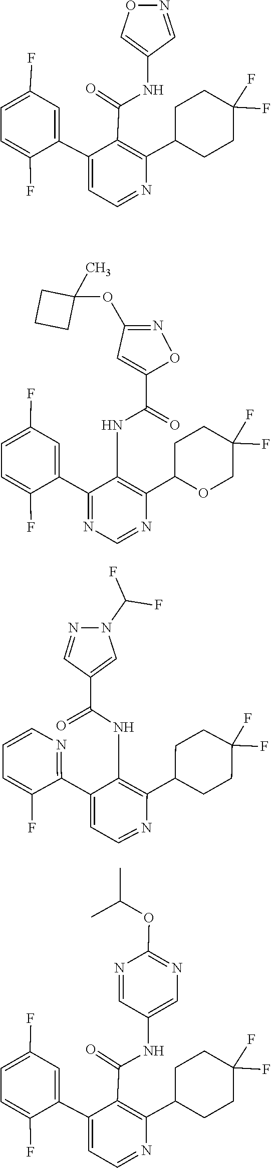 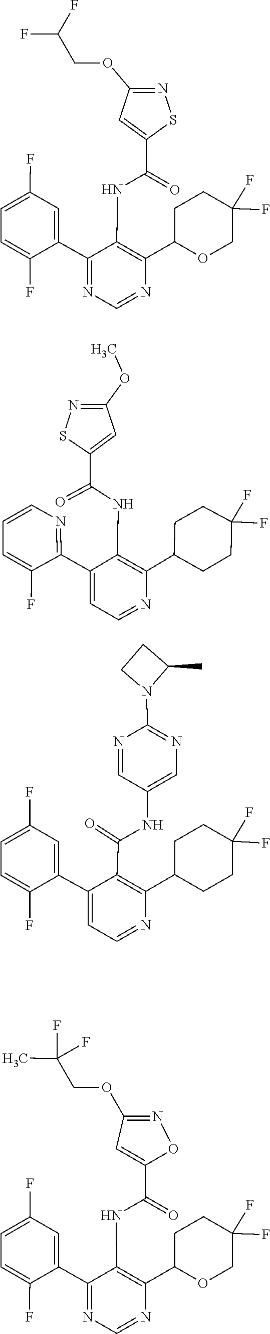 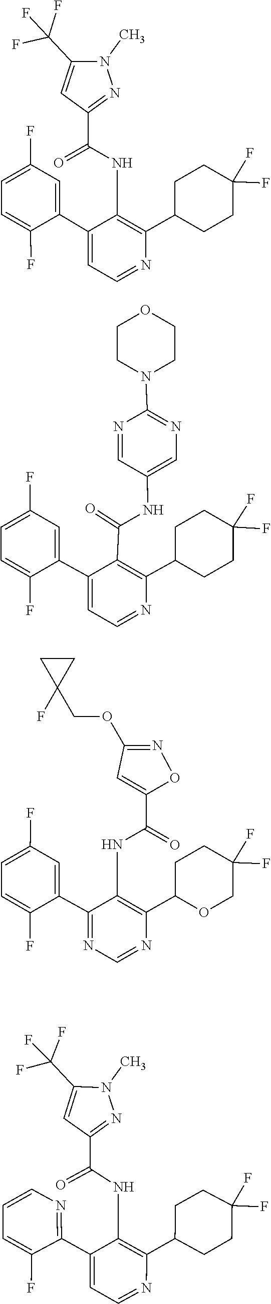 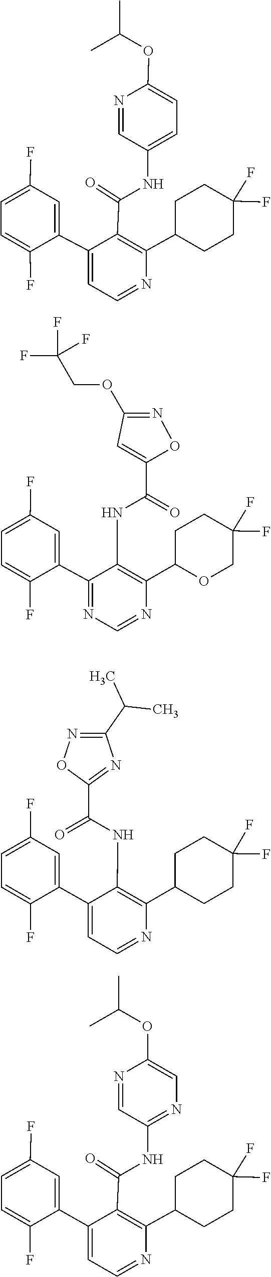 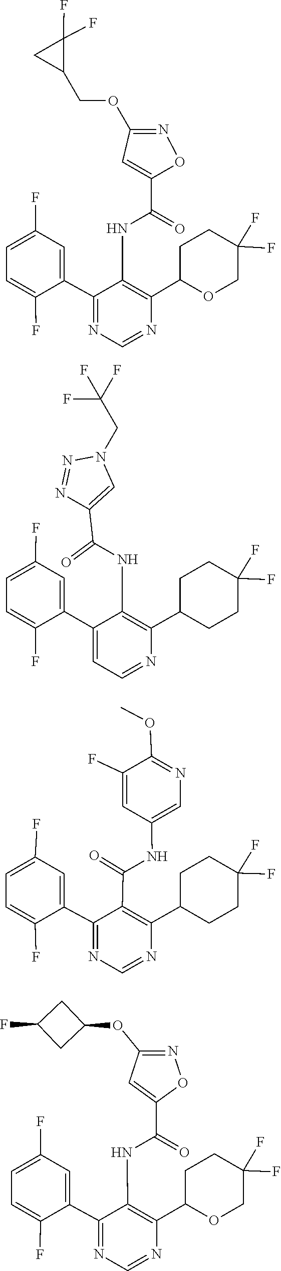 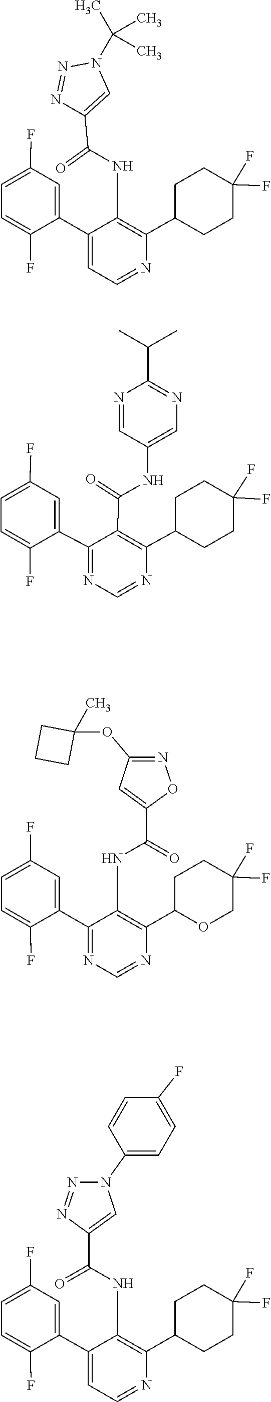 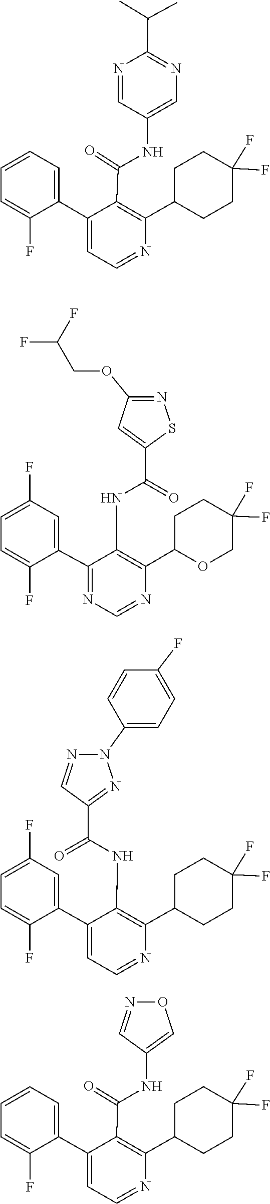 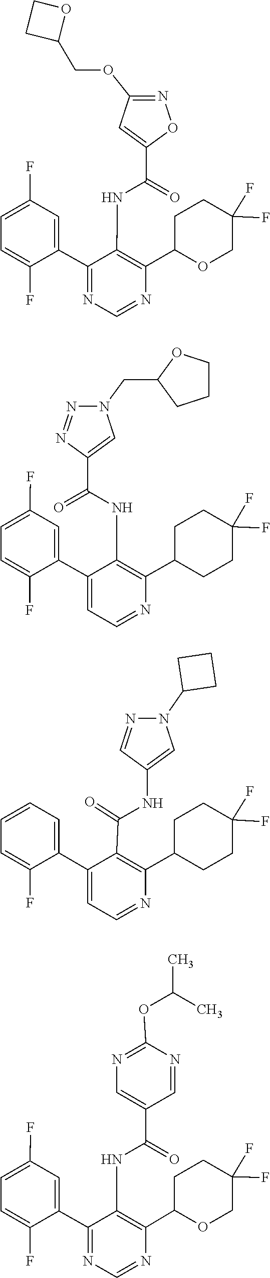 [SMILES: O=C(Nc1c(-c2cc(F)ccc2F)ncnc1C1CCC(F)(F)CO1)c1cc(OCC2CCO2)no1 ; O=C(Nc1c(-c2cc(F)ccc2F)ccnc1C1CCC(F)(F)CC1)c1cn(CC2CCCO2)nn1 ; O=C(Nc1cnn(C2CCC2)c1)c1c(-c2ccccc2F)ccnc1C1CCC(F)(F)CC1 ; CC(C)Oc1ncc(C(=O)Nc2c(-c3cc(F)ccc3F)ncnc2C2CCC(F)(F)CO2)cn1] 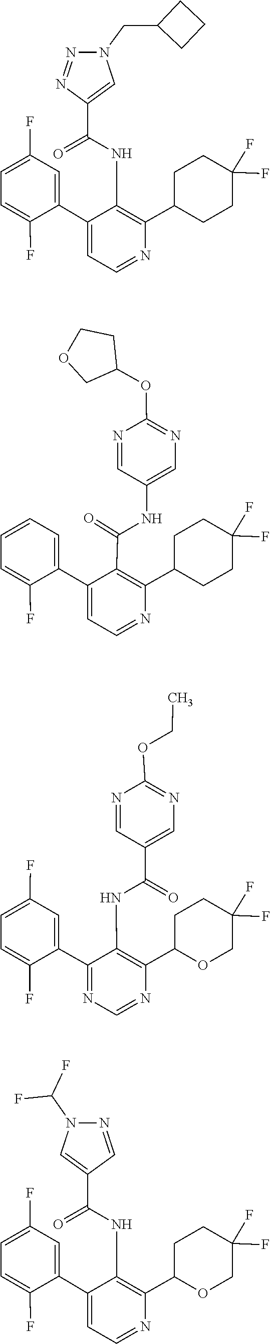 [SMILES: O=C(Nc1c(-c2cc(F)ccc2F)ccnc1C1CCC(F)(F)CC1)c1cn(CC2CCC2)nn1 ; O=C(Nc1cnc(OC2CCOC2)nc1)c1c(-c2ccccc2F)ccnc1C1CCC(F)(F)CC1 ; CCOc1ncc(C(=O)Nc2c(-c3cc(F)ccc3F)ncnc2C2CCC(F)(F)CO2)cn1 ; O=C(Nc1c(-c2cc(F)ccc2F)ccnc1C1CCC(F)(F)CO1)c1cnn(C(F)F)c1] 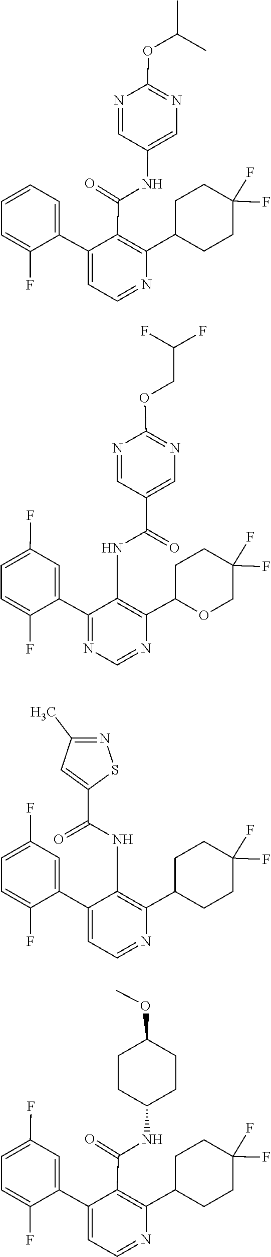 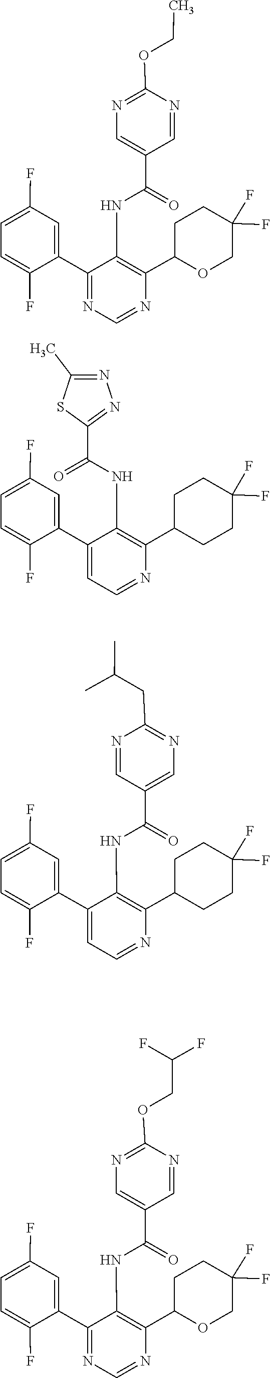 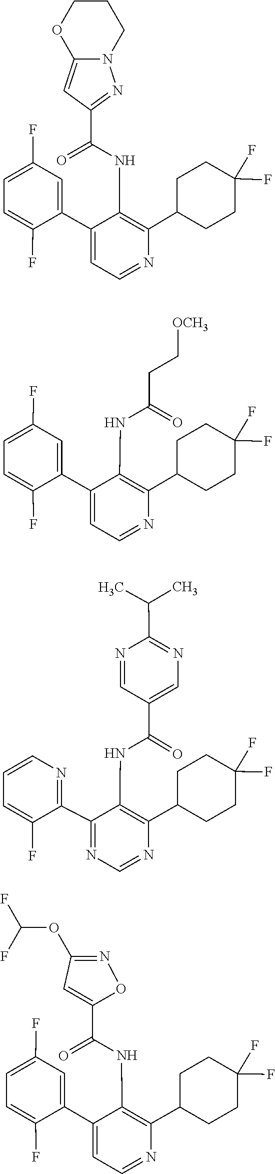 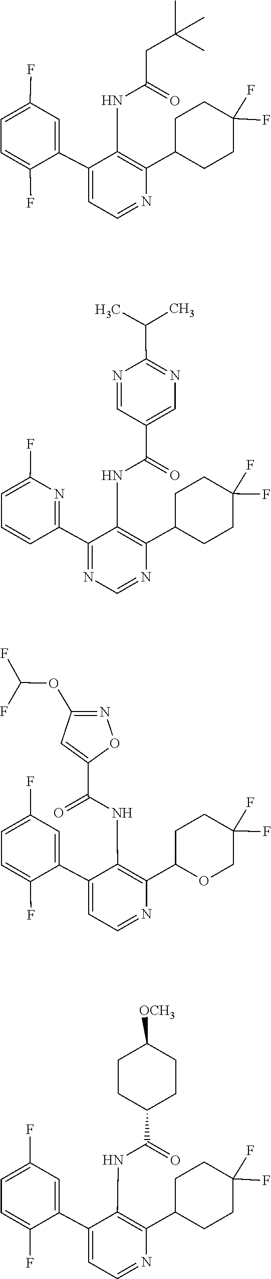 [SMILES: CC(C)(C)CC(=O)Nc1c(-c2cc(F)ccc2F)ccnc1C1CCC(F)(F)CC1 ; CC(C)c1ncc(C(=O)Nc2c(-c3cccc(F)n3)ncnc2C2CCC(F)(F)CC2)cn1 ; O=C(Nc1c(-c2cc(F)ccc2F)ccnc1C1CCC(F)(F)CO1)c1cc(OC(F)F)no1 ; CO[C@H]1CC[C@H](C(=O)Nc2c(-c3cc(F)ccc3F)ccnc2C2CCC(F)(F)CC2)CC1] 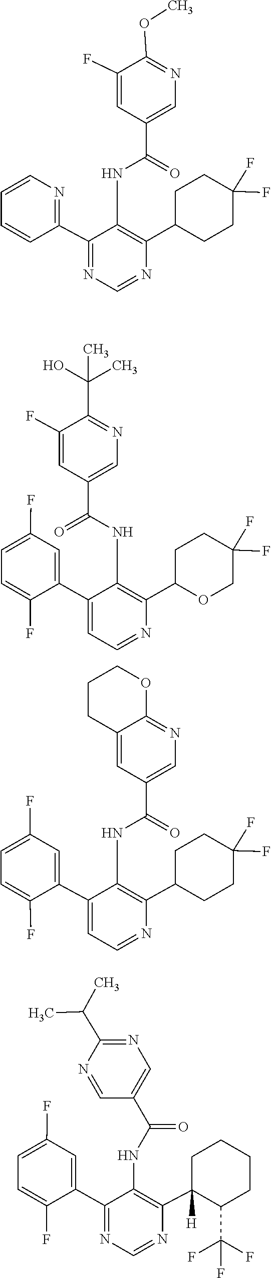 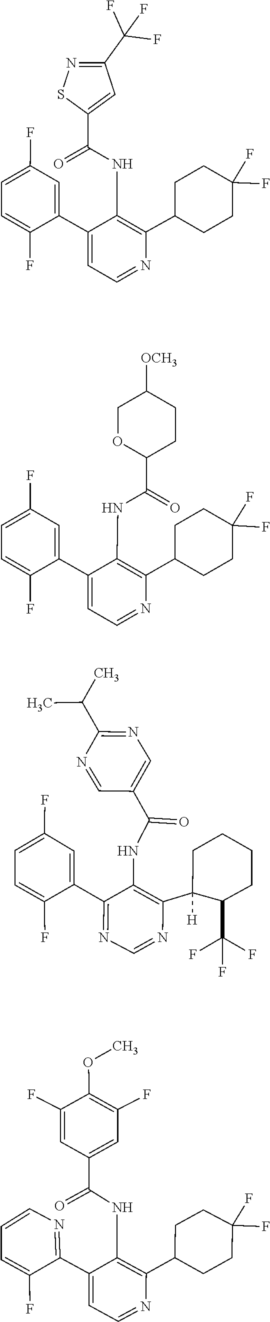 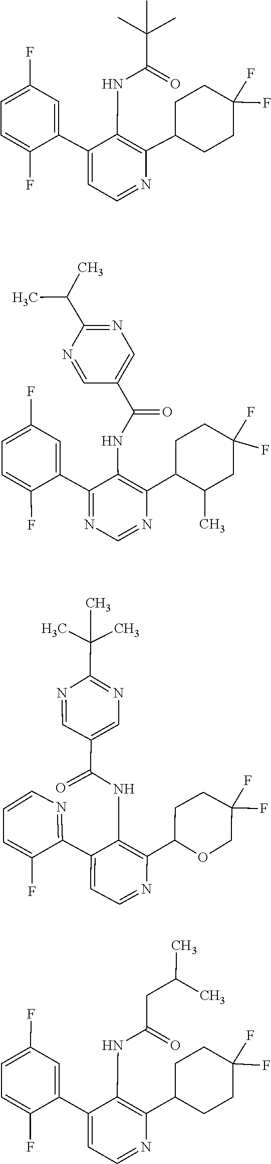 [SMILES: CC(C)(C)C(=O)Nc1c(-c2cc(F)ccc2F)ccnc1C1CCC(F)(F)CC1 ; CC(C)c1ncc(C(=O)Nc2c(-c3cc(F)ccc3F)ncnc2C2CCC(F)(F)CC2C)cn1 ; CC(C)(C)c1ncc(C(=O)Nc2c(-c3ncccc3F)ccnc2C2CCC(F)(F)CO2)cn1 ; CC(C)CC(=O)Nc1c(-c2cc(F)ccc2F)ccnc1C1CCC(F)(F)CC1] 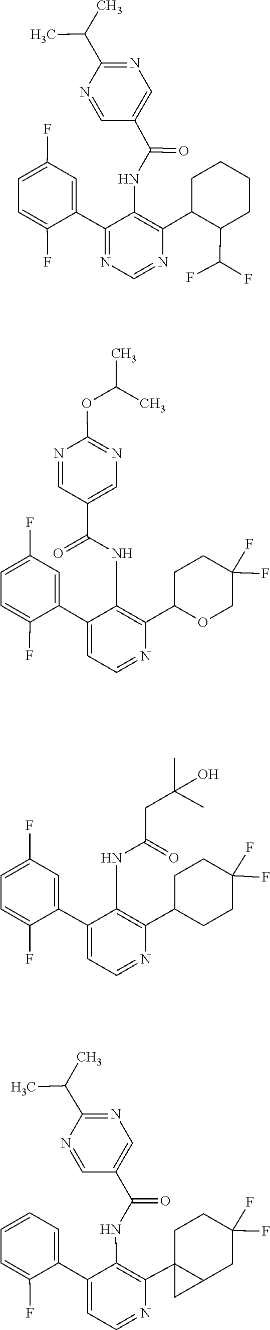 [SMILES: CC(C)c1ncc(C(=O)Nc2c(-c3cc(F)ccc3F)ncnc2C2CCCCC2C(F)F)cn1 ; CC(C)Oc1ncc(C(=O)Nc2c(-c3cc(F)ccc3F)ccnc2C2CCC(F)(F)CO2)cn1 ; CC(C)(O)CC(=O)Nc1c(-c2cc(F)ccc2F)ccnc1C1CCC(F)(F)CC1 ; CC(C)c1ncc(C(=O)Nc2c(-c3ccccc3F)ccnc2C23CCC(F)(F)CC2C3)cn1] 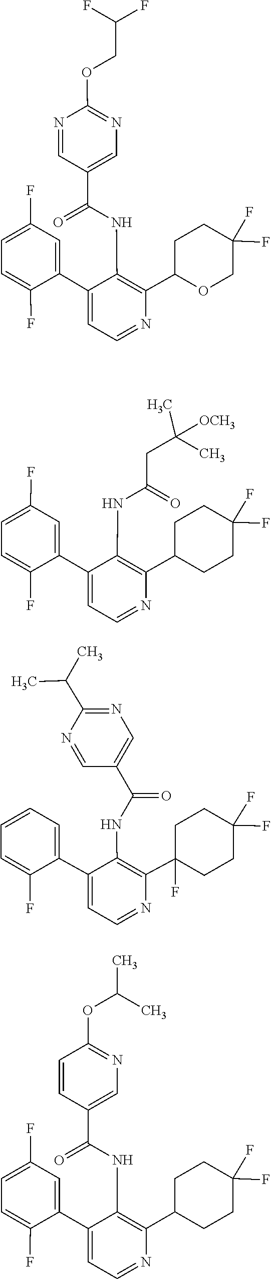 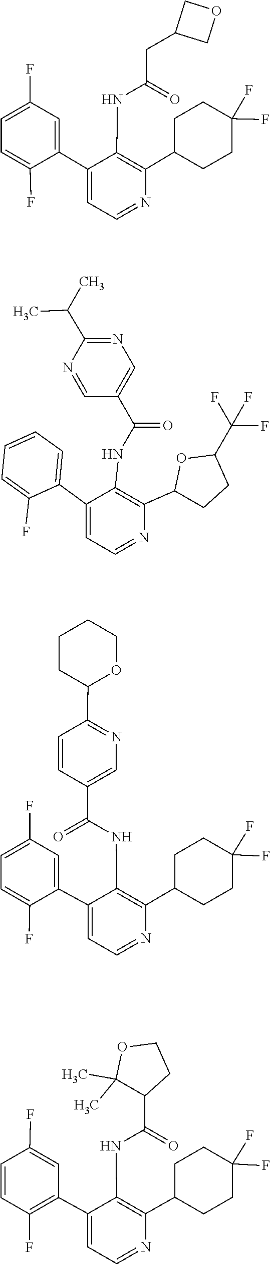 [SMILES: O=C(CC1COC1)Nc1c(-c2cc(F)ccc2F)ccnc1C1CCC(F)(F)CC1 ; CC(C)c1ncc(C(=O)Nc2c(-c3ccccc3F)ccnc2C2CCC(C(F)(F)F)O2)cn1 ; O=C(Nc1c(-c2cc(F)ccc2F)ccnc1C1CCC(F)(F)CC1)c1ccc(C2CCCCO2)nc1 ; CC1(C)OCCC1C(=O)Nc1c(-c2cc(F)ccc2F)ccnc1C1CCC(F)(F)CC1] 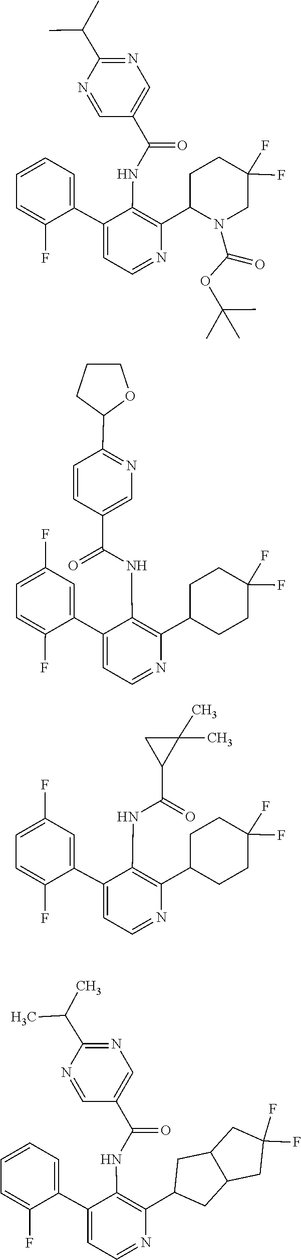 [SMILES: CC(C)c1ncc(C(=O)Nc2c(-c3ccccc3F)ccnc2C2CCC(F)(F)CN2C(=O)OC(C)(C)C)cn1 ; O=C(Nc1c(-c2cc(F)ccc2F)ccnc1C1CCC(F)(F)CC1)c1ccc(C2CCCO2)nc1 ; CC1(C)CC1C(=O)Nc1c(-c2cc(F)ccc2F)ccnc1C1CCC(F)(F)CC1 ; CC(C)c1ncc(C(=O)Nc2c(-c3ccccc3F)ccnc2C2CC3CC(F)(F)CC3C2)cn1] 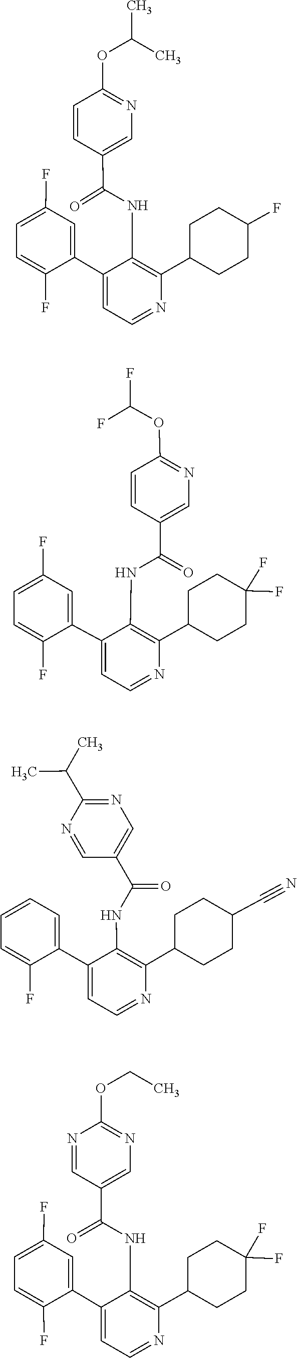 [SMILES: CC(C)Oc1ccc(C(=O)Nc2c(-c3cc(F)ccc3F)ccnc2C2CCC(F)CC2)cn1 ; O=C(Nc1c(-c2cc(F)ccc2F)ccnc1C1CCC(F)(F)CC1)c1ccc(OC(F)F)nc1 ; CC(C)c1ncc(C(=O)Nc2c(-c3ccccc3F)ccnc2C2CCC(C#N)CC2)cn1 ; CCOc1ncc(C(=O)Nc2c(-c3cc(F)ccc3F)ccnc2C2CCC(F)(F)CC2)cn1] 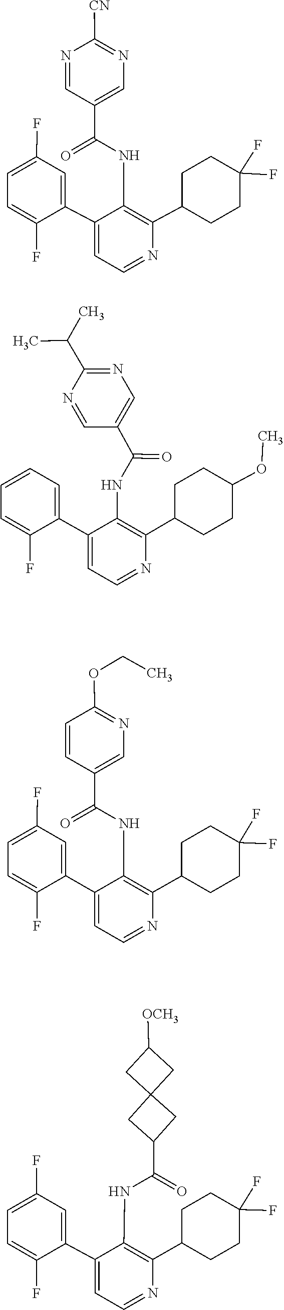 [SMILES: N#Cc1ncc(C(=O)Nc2c(-c3cc(F)ccc3F)ccnc2C2CCC(F)(F)CC2)cn1 ; COC1CCC(c2nccc(-c3ccccc3F)c2NC(=O)c2cnc(C(C)C)nc2)CC1 ; CCOc1ccc(C(=O)Nc2c(-c3cc(F)ccc3F)ccnc2C2CCC(F)(F)CC2)cn1 ; COC1CC2(C1)CC(C(=O)Nc1c(-c3cc(F)ccc3F)ccnc1C1CCC(F)(F)CC1)C2] 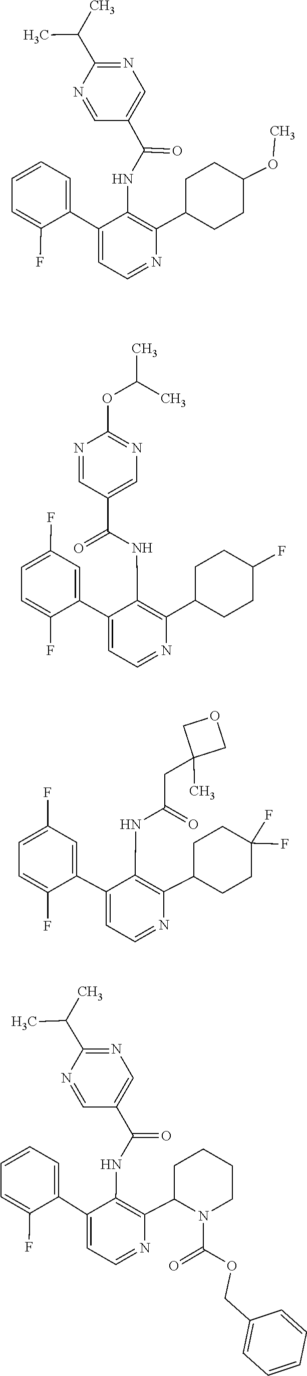 [SMILES: COC1CCC(c2nccc(-c3ccccc3F)c2NC(=O)c2cnc(C(C)C)nc2)CC1 ; CC(C)Oc1ncc(C(=O)Nc2c(-c3cc(F)ccc3F)ccnc2C2CCC(F)CC2)cn1 ; CC1(CC(=O)Nc2c(-c3cc(F)ccc3F)ccnc2C2CCC(F)(F)CC2)COC1 ; CC(C)c1ncc(C(=O)Nc2c(-c3ccccc3F)ccnc2C2CCCCN2C(=O)OCc2ccccc2)cn1] 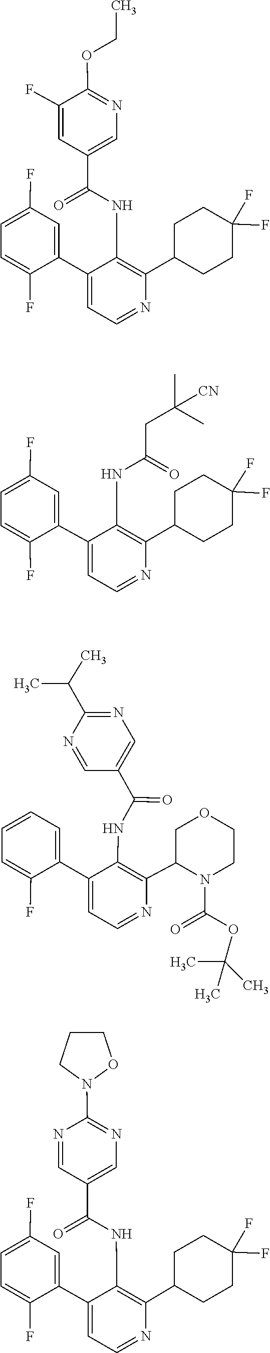 [SMILES: CCOc1ncc(C(=O)Nc2c(-c3cc(F)ccc3F)ccnc2C2CCC(F)(F)CC2)cc1F ; CC(C)(C#N)CC(=O)Nc1c(-c2cc(F)ccc2F)ccnc1C1CCC(F)(F)CC1 ; CC(C)c1ncc(C(=O)Nc2c(-c3ccccc3F)ccnc2C2COCCN2C(=O)OC(C)(C)C)cn1 ; O=C(Nc1c(-c2cc(F)ccc2F)ccnc1C1CCC(F)(F)CC1)c1cnc(N2CCCO2)nc1] 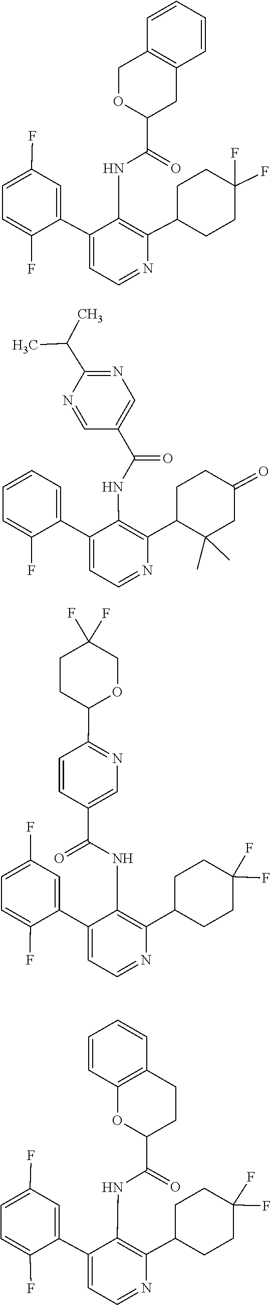 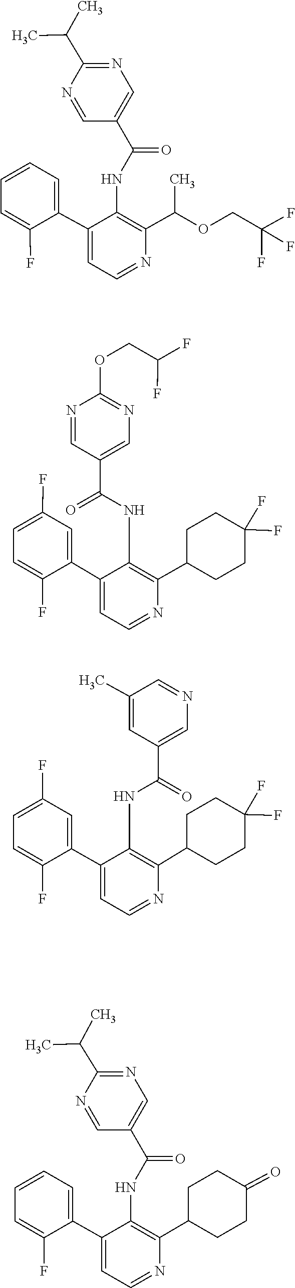 [SMILES: CC(C)c1ncc(C(=O)Nc2c(-c3ccccc3F)ccnc2C(C)OCC(F)(F)F)cn1 ; O=C(Nc1c(-c2cc(F)ccc2F)ccnc1C1CCC(F)(F)CC1)c1cnc(OCC(F)F)nc1 ; Cc1cncc(C(=O)Nc2c(-c3cc(F)ccc3F)ccnc2C2CCC(F)(F)CC2)c1 ; CC(C)c1ncc(C(=O)Nc2c(-c3ccccc3F)ccnc2C2CCC(=O)CC2)cn1] 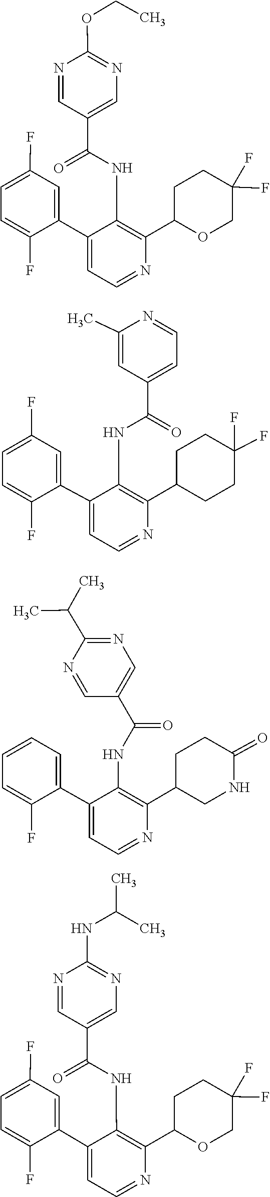 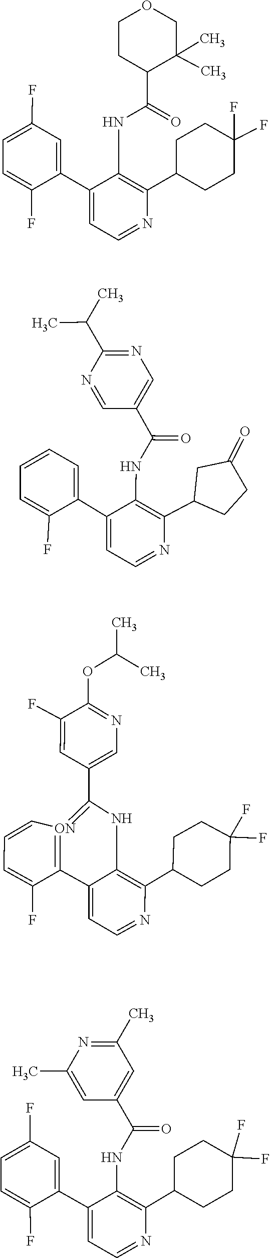 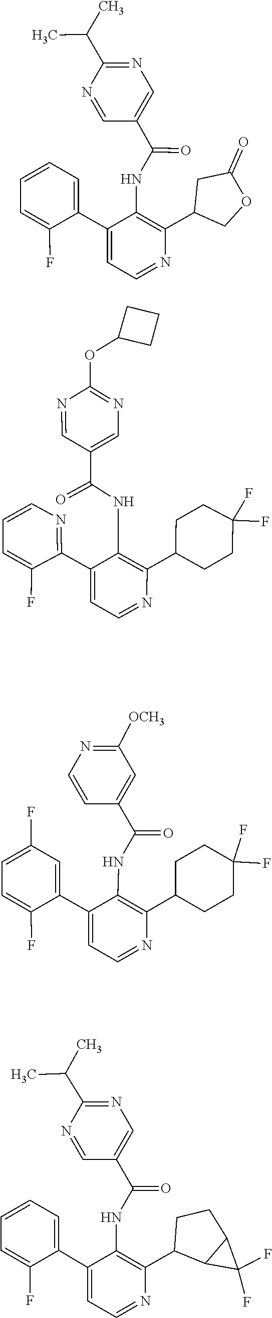 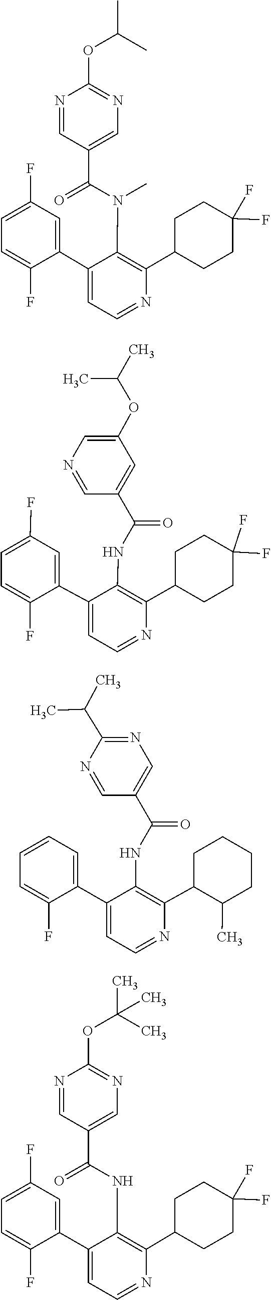 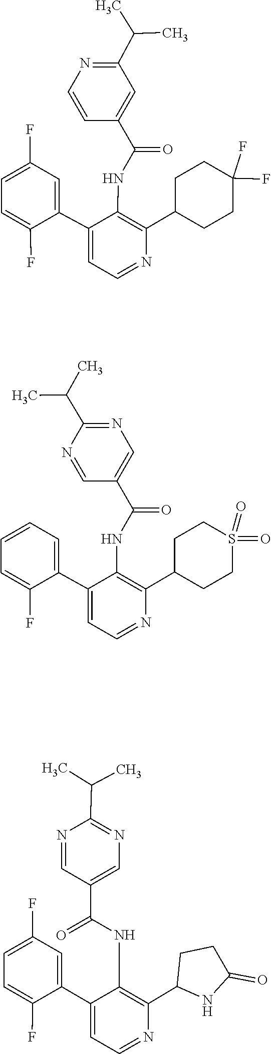 [SMILES: CC(C)c1cc(C(=O)Nc2c(-c3cc(F)ccc3F)ccnc2C2CCC(F)(F)CC2)ccn1 ; CC(C)c1ncc(C(=O)Nc2c(-c3ccccc3F)ccnc2C2CCS(=O)(=O)CC2)cn1 ; CC(C)c1ncc(C(=O)Nc2c(-c3cc(F)ccc3F)ccnc2C2CCC(=O)N2)cn1] 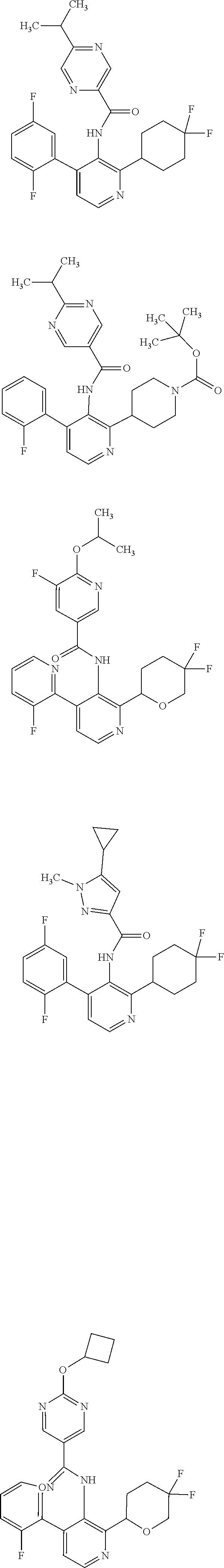 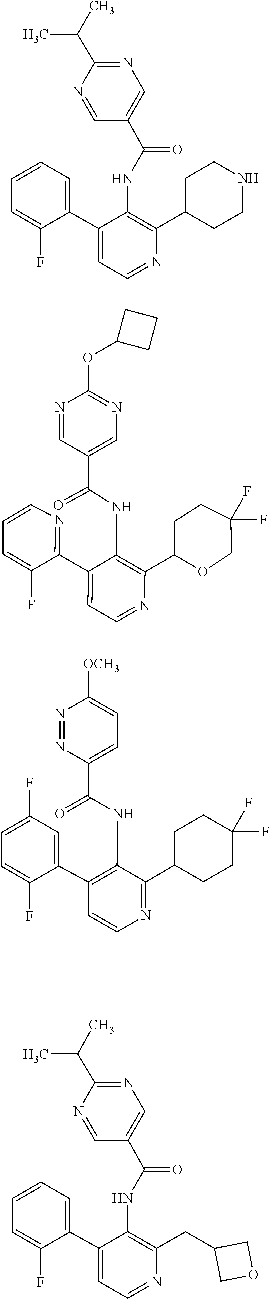 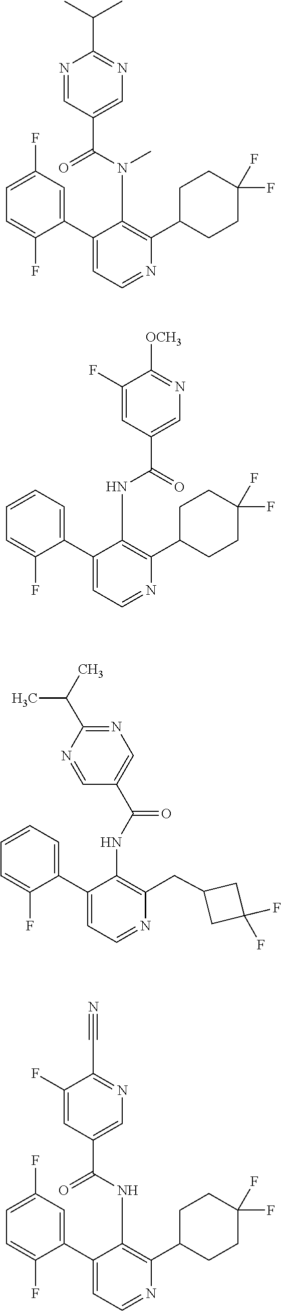 [SMILES: CC(C)c1ncc(C(=O)N(C)c2c(-c3cc(F)ccc3F)ccnc2C2CCC(F)(F)CC2)cn1 ; COc1ncc(C(=O)Nc2c(-c3ccccc3F)ccnc2C2CCC(F)(F)CC2)cc1F ; CC(C)c1ncc(C(=O)Nc2c(-c3ccccc3F)ccnc2CC2CC(F)(F)C2)cn1 ; N#Cc1ncc(C(=O)Nc2c(-c3cc(F)ccc3F)ccnc2C2CCC(F)(F)CC2)cc1F] 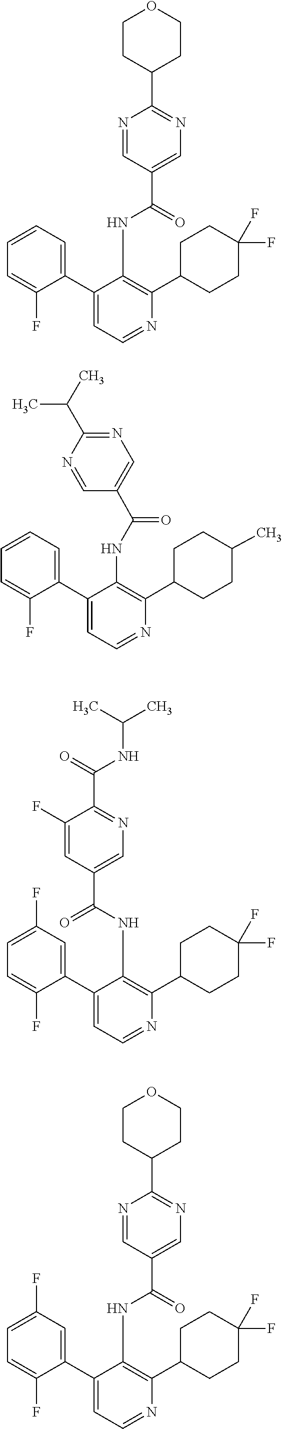 [SMILES: O=C(Nc1c(-c2ccccc2F)ccnc1C1CCC(F)(F)CC1)c1cnc(C2CCOCC2)nc1 ; CC1CCC(c2nccc(-c3ccccc3F)c2NC(=O)c2cnc(C(C)C)nc2)CC1 ; CC(C)NC(=O)c1ncc(C(=O)Nc2c(-c3cc(F)ccc3F)ccnc2C2CCC(F)(F)CC2)cc1F ; O=C(Nc1c(-c2cc(F)ccc2F)ccnc1C1CCC(F)(F)CC1)c1cnc(C2CCOCC2)nc1] 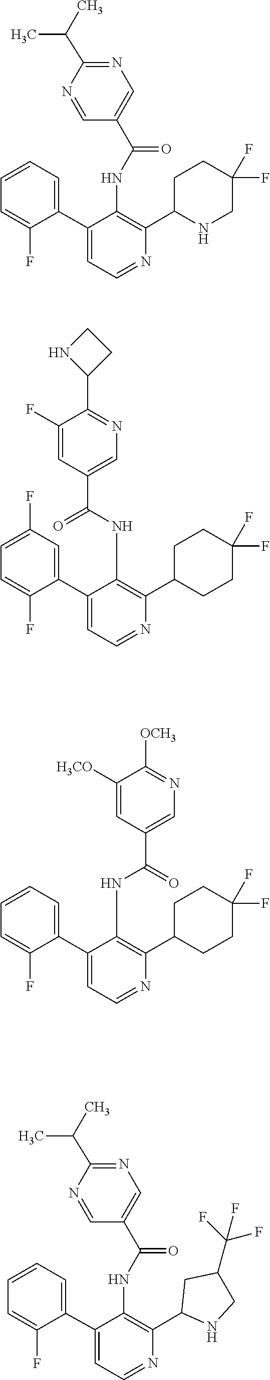 [SMILES: CC(C)c1ncc(C(=O)Nc2c(-c3ccccc3F)ccnc2C2CCC(F)(F)CN2)cn1 ; O=C(Nc1c(-c2cc(F)ccc2F)ccnc1C1CCC(F)(F)CC1)c1cnc(C2CCN2)c(F)c1 ; COc1cc(C(=O)Nc2c(-c3ccccc3F)ccnc2C2CCC(F)(F)CC2)cnc1OC ; CC(C)c1ncc(C(=O)Nc2c(-c3ccccc3F)ccnc2C2CC(C(F)(F)F)CN2)cn1] 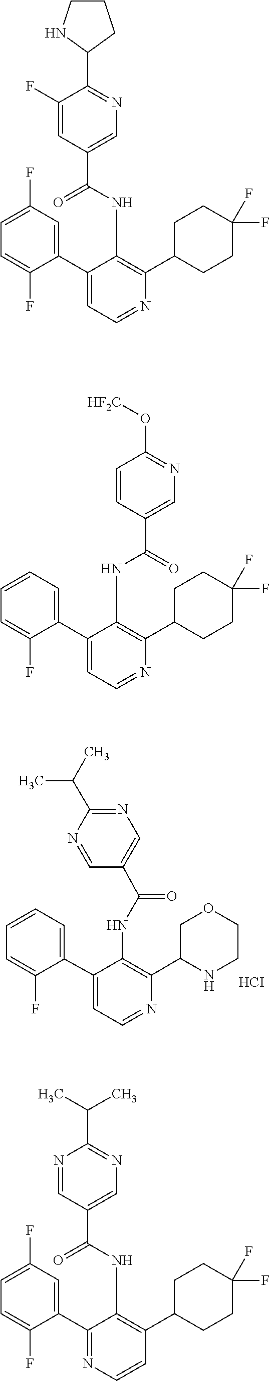 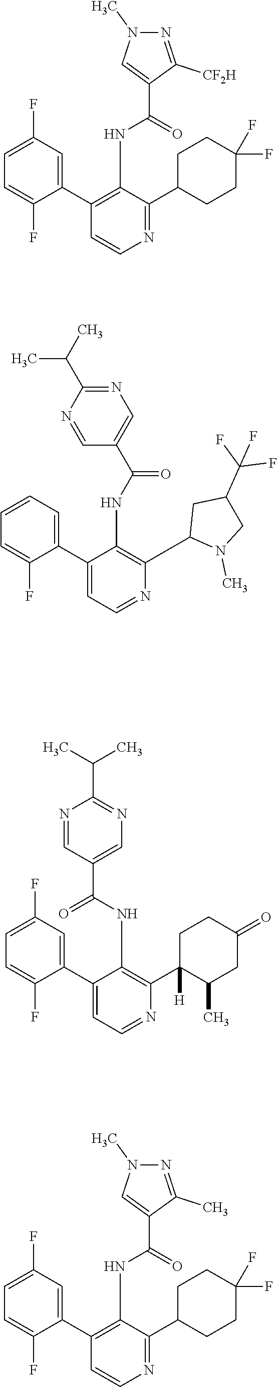 [SMILES: Cn1cc(C(=O)Nc2c(-c3cc(F)ccc3F)ccnc2C2CCC(F)(F)CC2)c(C(F)F)n1 ; CC(C)c1ncc(C(=O)Nc2c(-c3ccccc3F)ccnc2C2CC(C(F)(F)F)CN2C)cn1 ; CC(C)c1ncc(C(=O)Nc2c(-c3cc(F)ccc3F)ccnc2[C@@H]2CCC(=O)C[C@H]2C)cn1 ; Cc1nn(C)cc1C(=O)Nc1c(-c2cc(F)ccc2F)ccnc1C1CCC(F)(F)CC1] 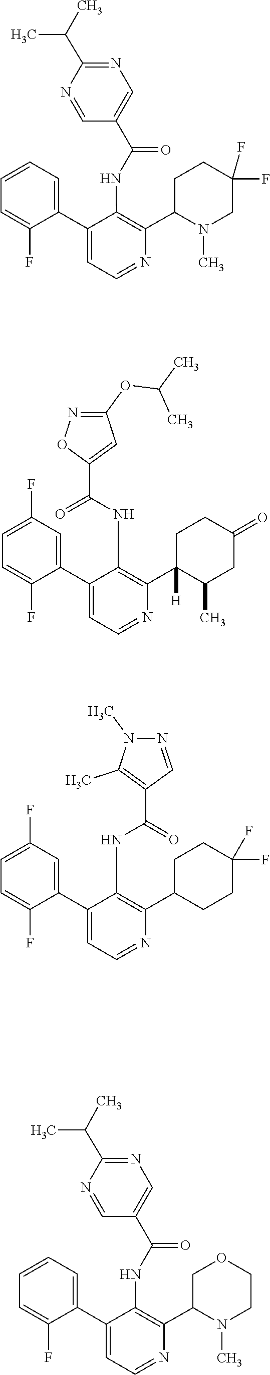 [SMILES: CC(C)c1ncc(C(=O)Nc2c(-c3ccccc3F)ccnc2C2CCC(F)(F)CN2C)cn1 ; CC(C)Oc1cc(C(=O)Nc2c(-c3cc(F)ccc3F)ccnc2[C@@H]2CCC(=O)C[C@H]2C)on1 ; Cc1c(C(=O)Nc2c(-c3cc(F)ccc3F)ccnc2C2CCC(F)(F)CC2)cnn1C ; CC(C)c1ncc(C(=O)Nc2c(-c3ccccc3F)ccnc2C2COCCN2C)cn1] 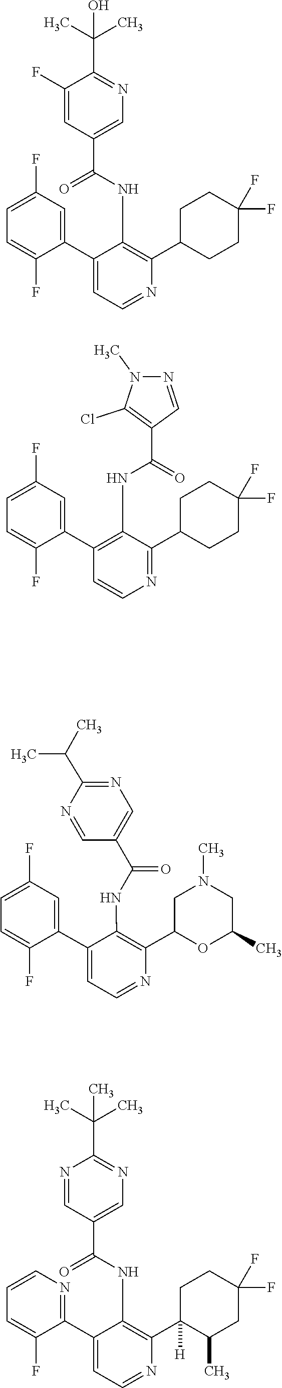 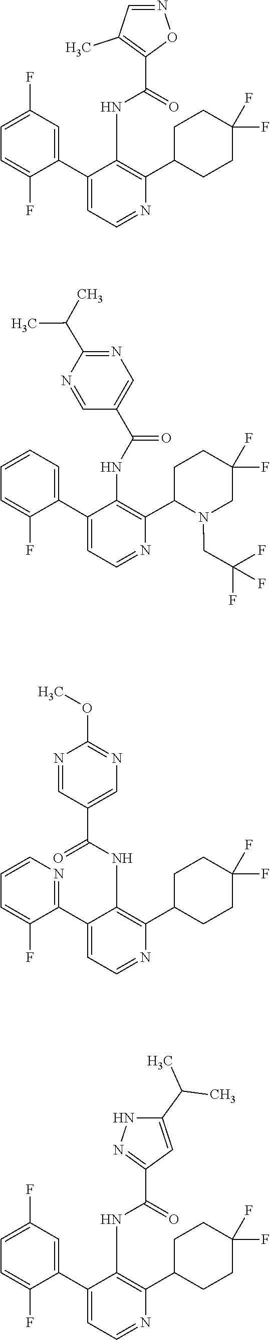 [SMILES: Cc1cnoc1C(=O)Nc1c(-c2cc(F)ccc2F)ccnc1C1CCC(F)(F)CC1 ; CC(C)c1ncc(C(=O)Nc2c(-c3ccccc3F)ccnc2C2CCC(F)(F)CN2CC(F)(F)F)cn1 ; COc1ncc(C(=O)Nc2c(-c3ncccc3F)ccnc2C2CCC(F)(F)CC2)cn1 ; CC(C)c1cc(C(=O)Nc2c(-c3cc(F)ccc3F)ccnc2C2CCC(F)(F)CC2)n[nH]1] 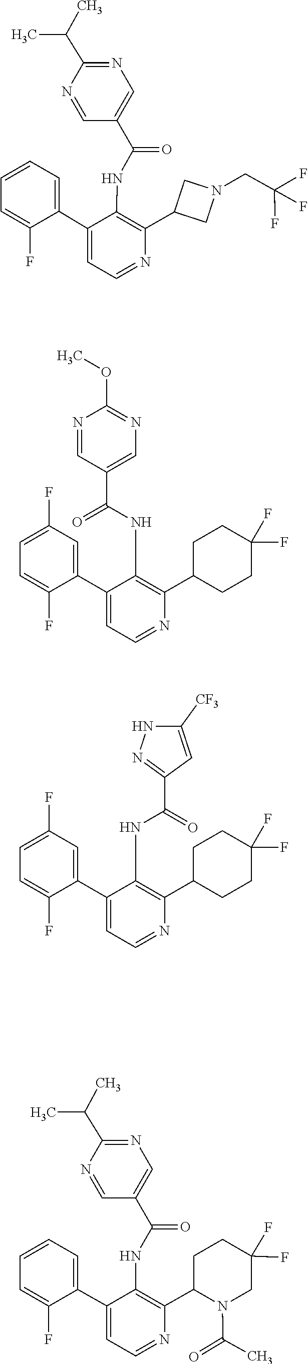 [SMILES: CC(C)c1ncc(C(=O)Nc2c(-c3ccccc3F)ccnc2C2CN(CC(F)(F)F)C2)cn1 ; COc1ncc(C(=O)Nc2c(-c3cc(F)ccc3F)ccnc2C2CCC(F)(F)CC2)cn1 ; O=C(Nc1c(-c2cc(F)ccc2F)ccnc1C1CCC(F)(F)CC1)c1cc(C(F)(F)F)[nH]n1 ; CC(=O)N1CC(F)(F)CCC1c1nccc(-c2ccccc2F)c1NC(=O)c1cnc(C(C)C)nc1] 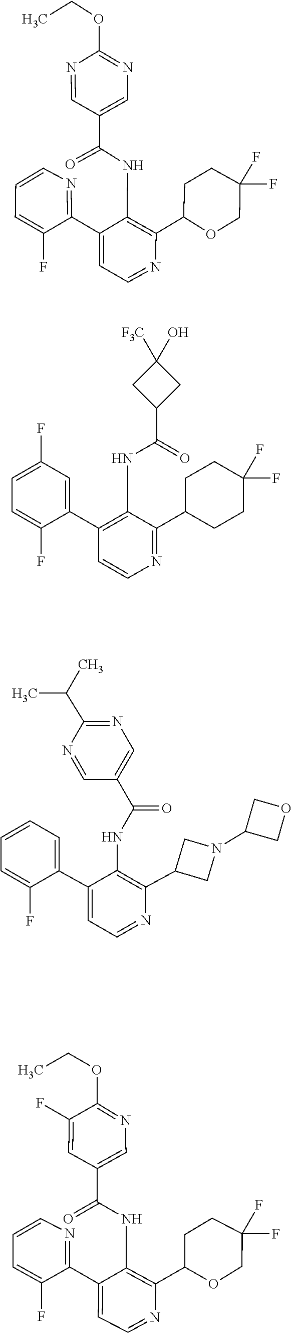 [SMILES: CCOc1ncc(C(=O)Nc2c(-c3ncccc3F)ccnc2C2CCC(F)(F)CO2)cn1 ; O=C(Nc1c(-c2cc(F)ccc2F)ccnc1C1CCC(F)(F)CC1)C1CC(O)(C(F)(F)F)C1 ; CC(C)c1ncc(C(=O)Nc2c(-c3ccccc3F)ccnc2C2CN(C3COC3)C2)cn1 ; CCOc1ncc(C(=O)Nc2c(-c3ncccc3F)ccnc2C2CCC(F)(F)CO2)cc1F] 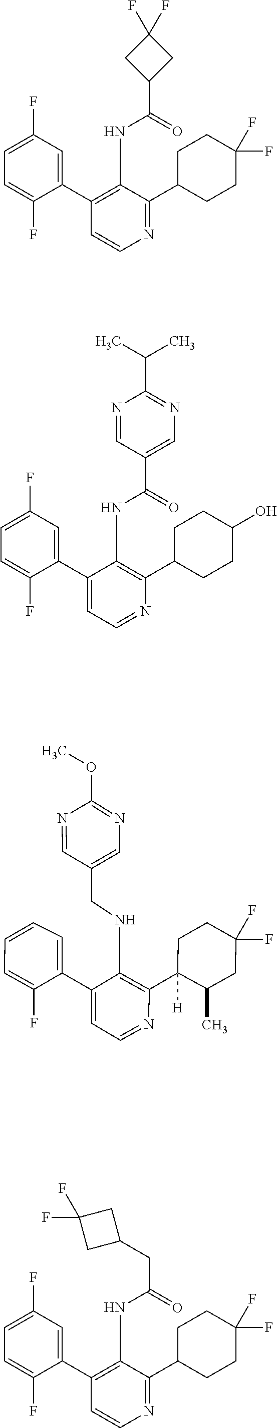 [SMILES: O=C(Nc1c(-c2cc(F)ccc2F)ccnc1C1CCC(F)(F)CC1)C1CC(F)(F)C1 ; CC(C)c1ncc(C(=O)Nc2c(-c3cc(F)ccc3F)ccnc2C2CCC(O)CC2)cn1 ; COc1ncc(CNc2c(-c3ccccc3F)ccnc2[C@H]2CCC(F)(F)C[C@H]2C)cn1 ; O=C(CC1CC(F)(F)C1)Nc1c(-c2cc(F)ccc2F)ccnc1C1CCC(F)(F)CC1] 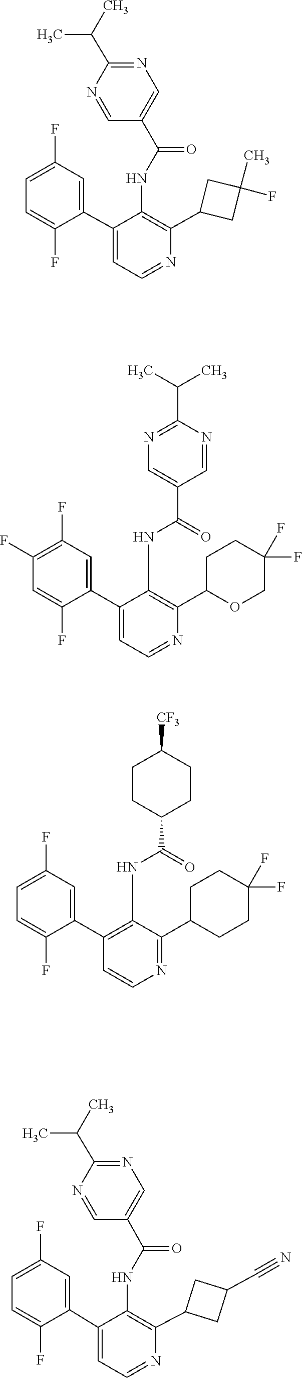 [SMILES: CC(C)c1ncc(C(=O)Nc2c(-c3cc(F)ccc3F)ccnc2C2CC(C)(F)C2)cn1 ; CC(C)c1ncc(C(=O)Nc2c(-c3cc(F)c(F)cc3F)ccnc2C2CCC(F)(F)CO2)cn1 ; O=C(Nc1c(-c2cc(F)ccc2F)ccnc1C1CCC(F)(F)CC1)[C@H]1CC[C@H](C(F)(F)F)CC1 ; CC(C)c1ncc(C(=O)Nc2c(-c3cc(F)ccc3F)ccnc2C2CC(C#N)C2)cn1] 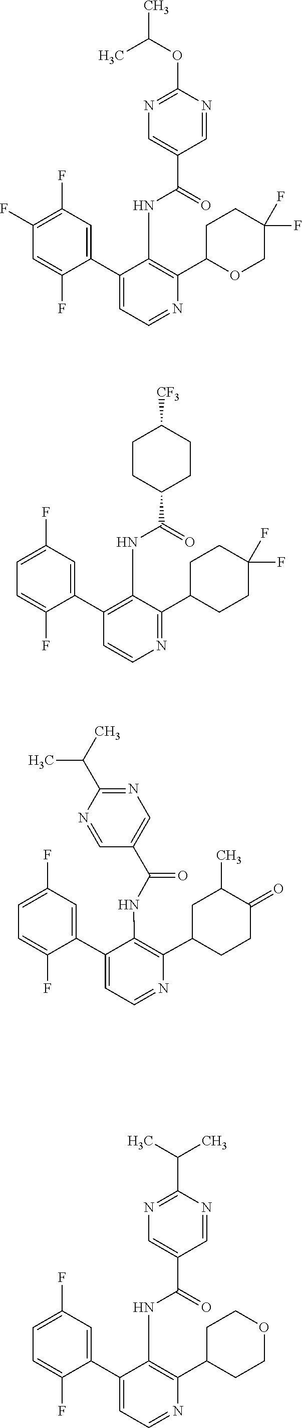 [SMILES: CC(C)Oc1ncc(C(=O)Nc2c(-c3cc(F)c(F)cc3F)ccnc2C2CCC(F)(F)CO2)cn1 ; O=C(Nc1c(-c2cc(F)ccc2F)ccnc1C1CCC(F)(F)CC1)[C@H]1CC[C@@H](C(F)(F)F)CC1 ; CC1CC(c2nccc(-c3cc(F)ccc3F)c2NC(=O)c2cnc(C(C)C)nc2)CCC1=O ; CC(C)c1ncc(C(=O)Nc2c(-c3cc(F)ccc3F)ccnc2C2CCOCC2)cn1] 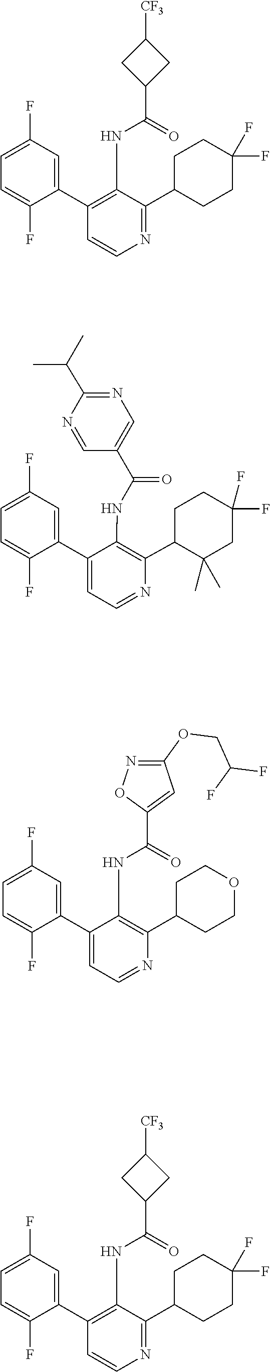 [SMILES: O=C(Nc1c(-c2cc(F)ccc2F)ccnc1C1CCC(F)(F)CC1)C1CC(C(F)(F)F)C1 ; CC(C)c1ncc(C(=O)Nc2c(-c3cc(F)ccc3F)ccnc2C2CCC(F)(F)CC2(C)C)cn1 ; O=C(Nc1c(-c2cc(F)ccc2F)ccnc1C1CCOCC1)c1cc(OCC(F)F)no1 ; O=C(Nc1c(-c2cc(F)ccc2F)ccnc1C1CCC(F)(F)CC1)C1CC(C(F)(F)F)C1] 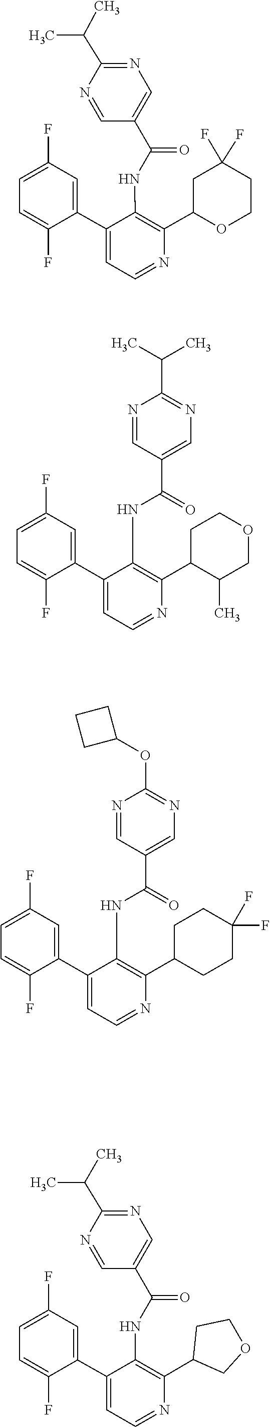 [SMILES: CC(C)c1ncc(C(=O)Nc2c(-c3cc(F)ccc3F)ccnc2C2CC(F)(F)CCO2)cn1 ; CC(C)c1ncc(C(=O)Nc2c(-c3cc(F)ccc3F)ccnc2C2CCOCC2C)cn1 ; O=C(Nc1c(-c2cc(F)ccc2F)ccnc1C1CCC(F)(F)CC1)c1cnc(OC2CCC2)nc1 ; CC(C)c1ncc(C(=O)Nc2c(-c3cc(F)ccc3F)ccnc2C2CCOC2)cn1] 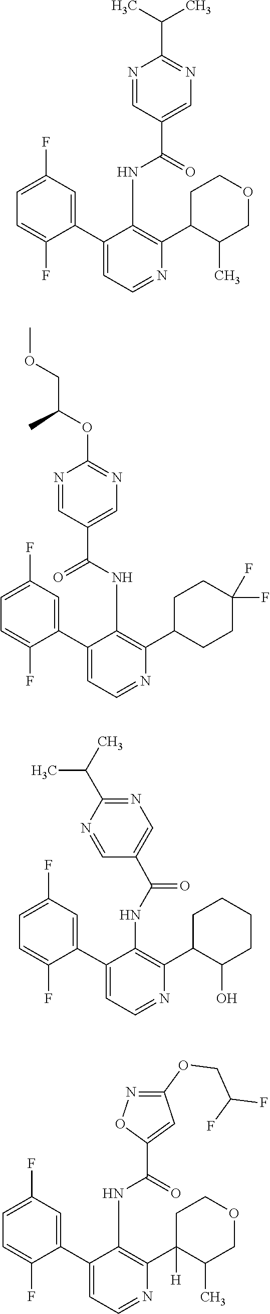 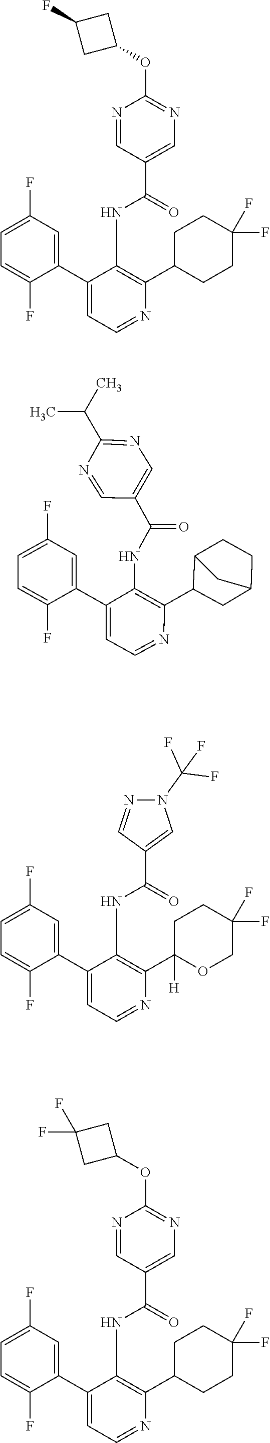 [SMILES: O=C(Nc1c(-c2cc(F)ccc2F)ccnc1C1CCC(F)(F)CC1)c1cnc(O[C@H]2C[C@H](F)C2)nc1 ; CC(C)c1ncc(C(=O)Nc2c(-c3cc(F)ccc3F)ccnc2C2CC3CCC2C3)cn1 ; O=C(Nc1c(-c2cc(F)ccc2F)ccnc1C1CCC(F)(F)CO1)c1cnn(C(F)(F)F)c1 ; O=C(Nc1c(-c2cc(F)ccc2F)ccnc1C1CCC(F)(F)CC1)c1cnc(OC2CC(F)(F)C2)nc1] 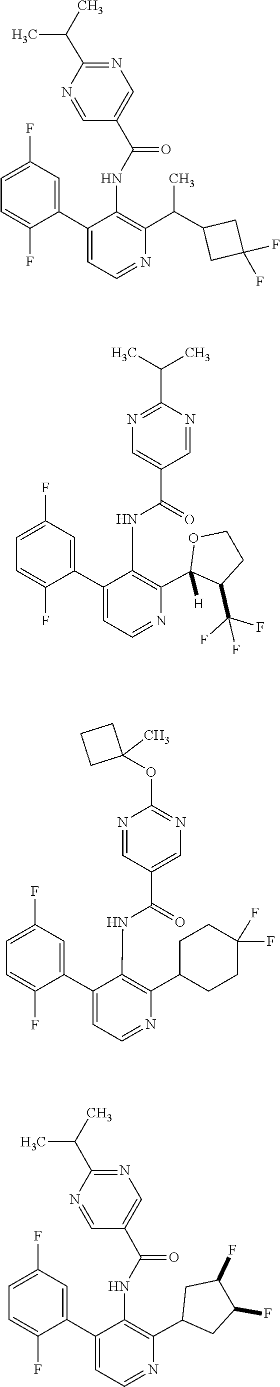 [SMILES: CC(C)c1ncc(C(=O)Nc2c(-c3cc(F)ccc3F)ccnc2C(C)C2CC(F)(F)C2)cn1 ; CC(C)c1ncc(C(=O)Nc2c(-c3cc(F)ccc3F)ccnc2[C@@H]2OCC[C@H]2C(F)(F)F)cn1 ; CC1(Oc2ncc(C(=O)Nc3c(-c4cc(F)ccc4F)ccnc3C3CCC(F)(F)CC3)cn2)CCC1 ; CC(C)c1ncc(C(=O)Nc2c(-c3cc(F)ccc3F)ccnc2C2C[C@@H](F)[C@@H](F)C2)cn1] 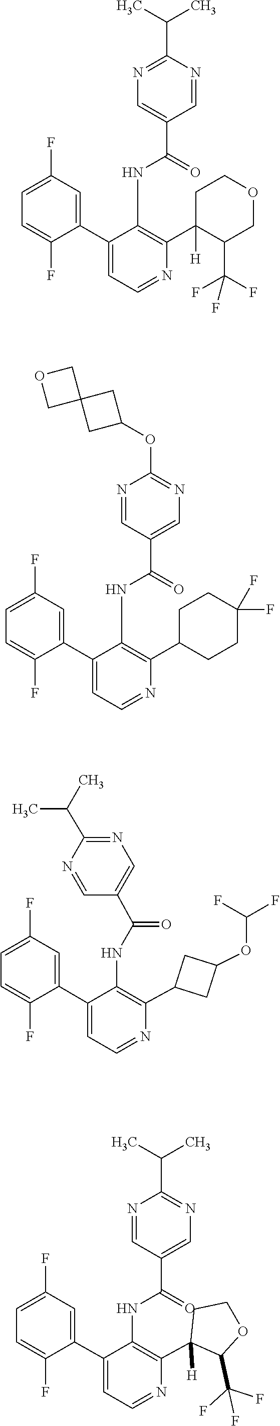 [SMILES: CC(C)c1ncc(C(=O)Nc2c(-c3cc(F)ccc3F)ccnc2C2CCOCC2C(F)(F)F)cn1 ; O=C(Nc1c(-c2cc(F)ccc2F)ccnc1C1CCC(F)(F)CC1)c1cnc(OC2CC3(COC3)C2)nc1 ; CC(C)c1ncc(C(=O)Nc2c(-c3cc(F)ccc3F)ccnc2C2CC(OC(F)F)C2)cn1 ; CC(C)c1ncc(C(=O)Nc2c(-c3cc(F)ccc3F)ccnc2[C@@H]2CCO[C@H]2C(F)(F)F)cn1] 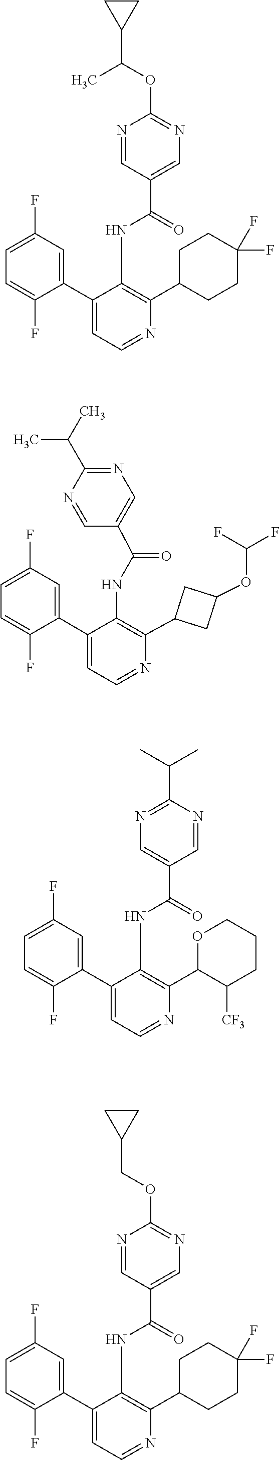 [SMILES: CC(Oc1ncc(C(=O)Nc2c(-c3cc(F)ccc3F)ccnc2C2CCC(F)(F)CC2)cn1)C1CC1 ; CC(C)c1ncc(C(=O)Nc2c(-c3cc(F)ccc3F)ccnc2C2CC(OC(F)F)C2)cn1 ; CC(C)c1ncc(C(=O)Nc2c(-c3cc(F)ccc3F)ccnc2C2OCCCC2C(F)(F)F)cn1 ; O=C(Nc1c(-c2cc(F)ccc2F)ccnc1C1CCC(F)(F)CC1)c1cnc(OCC2CC2)nc1] 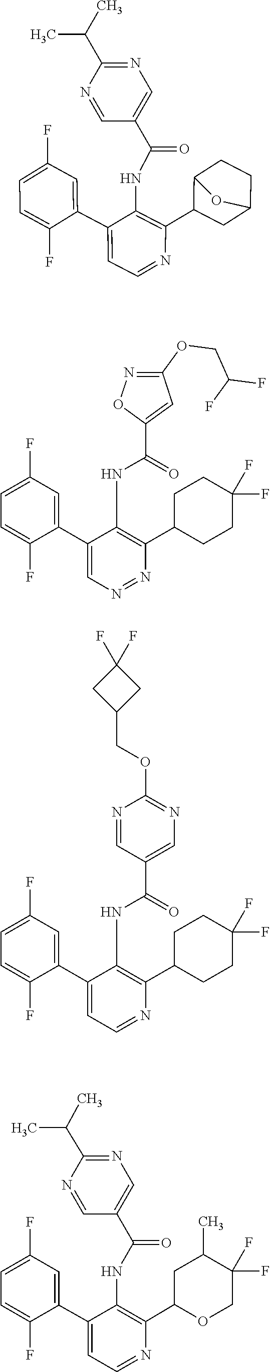 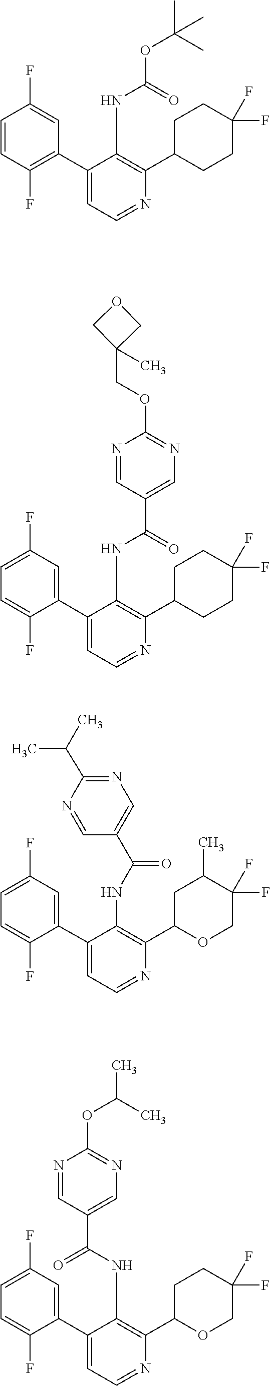 [SMILES: CC(C)(C)OC(=O)Nc1c(-c2cc(F)ccc2F)ccnc1C1CCC(F)(F)CC1 ; CC1(COc2ncc(C(=O)Nc3c(-c4cc(F)ccc4F)ccnc3C3CCC(F)(F)CC3)cn2)COC1 ; CC(C)c1ncc(C(=O)Nc2c(-c3cc(F)ccc3F)ccnc2C2CC(C)C(F)(F)CO2)cn1 ; CC(C)Oc1ncc(C(=O)Nc2c(-c3cc(F)ccc3F)ccnc2C2CCC(F)(F)CO2)cn1] 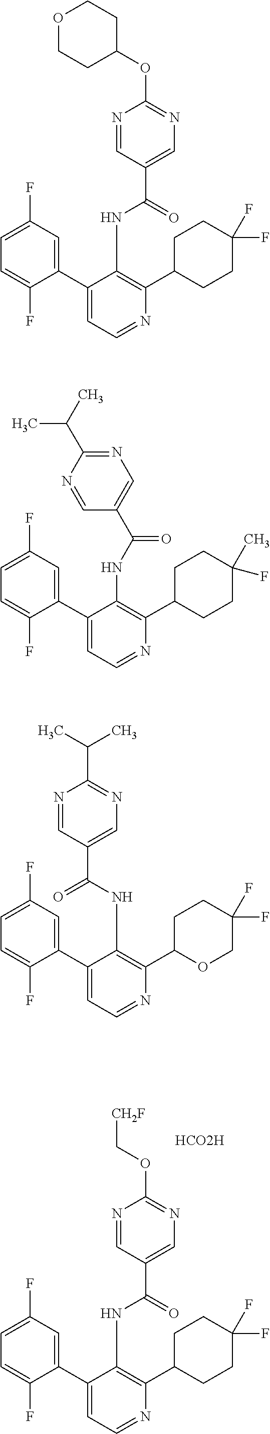 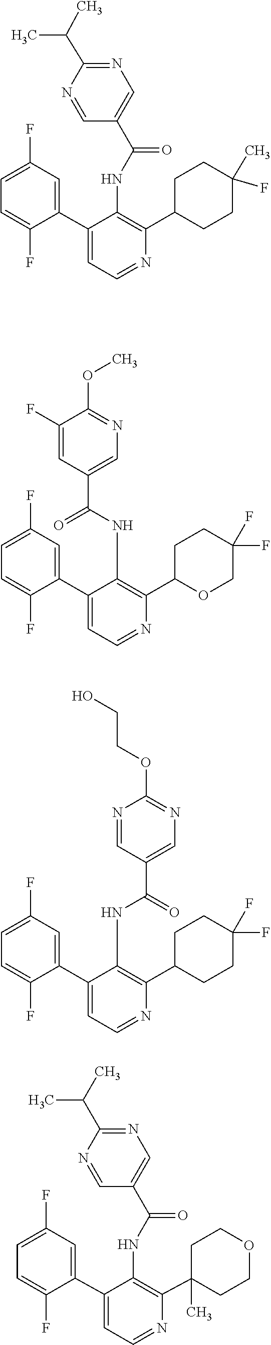 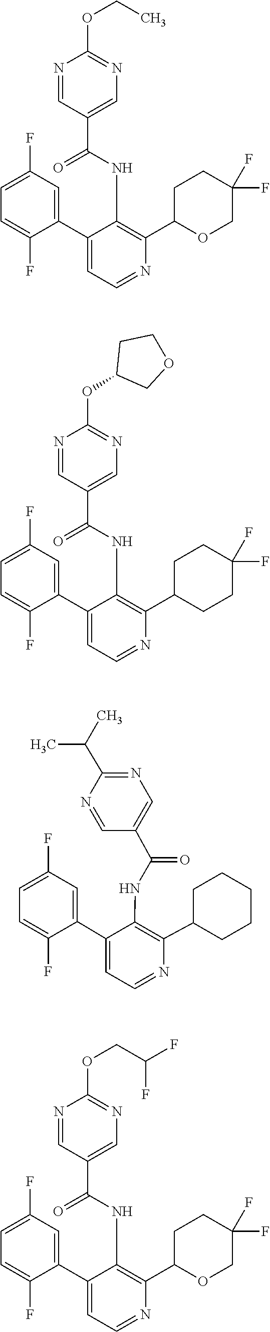 [SMILES: CCOc1ncc(C(=O)Nc2c(-c3cc(F)ccc3F)ccnc2C2CCC(F)(F)CO2)cn1 ; O=C(Nc1c(-c2cc(F)ccc2F)ccnc1C1CCC(F)(F)CC1)c1cnc(O[C@@H]2CCOC2)nc1 ; CC(C)c1ncc(C(=O)Nc2c(-c3cc(F)ccc3F)ccnc2C2CCCCC2)cn1 ; O=C(Nc1c(-c2cc(F)ccc2F)ccnc1C1CCC(F)(F)CO1)c1cnc(OCC(F)F)nc1] 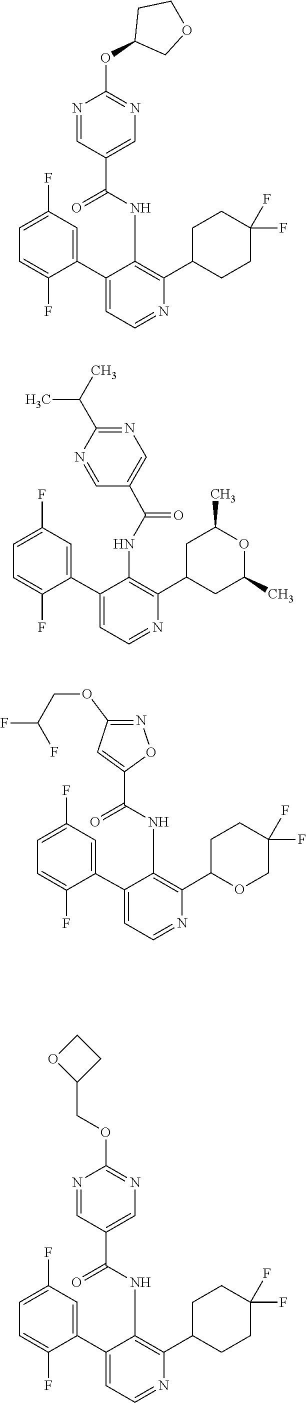 [SMILES: O=C(Nc1c(-c2cc(F)ccc2F)ccnc1C1CCC(F)(F)CC1)c1cnc(O[C@H]2CCOC2)nc1 ; CC(C)c1ncc(C(=O)Nc2c(-c3cc(F)ccc3F)ccnc2C2C[C@@H](C)O[C@@H](C)C2)cn1 ; O=C(Nc1c(-c2cc(F)ccc2F)ccnc1C1CCC(F)(F)CO1)c1cc(OCC(F)F)no1 ; O=C(Nc1c(-c2cc(F)ccc2F)ccnc1C1CCC(F)(F)CC1)c1cnc(OCC2CCO2)nc1] 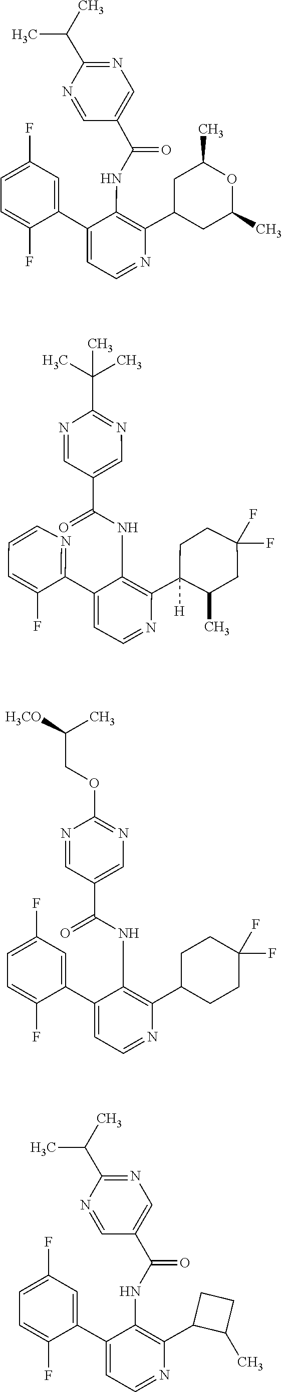 [SMILES: CC(C)c1ncc(C(=O)Nc2c(-c3cc(F)ccc3F)ccnc2C2C[C@@H](C)O[C@@H](C)C2)cn1 ; C[C@@H]1CC(F)(F)CC[C@@H]1c1nccc(-c2ncccc2F)c1NC(=O)c1cnc(C(C)(C)C)nc1 ; CO[C@@H](C)COc1ncc(C(=O)Nc2c(-c3cc(F)ccc3F)ccnc2C2CCC(F)(F)CC2)cn1 ; CC(C)c1ncc(C(=O)Nc2c(-c3cc(F)ccc3F)ccnc2C2CCC2C)cn1] 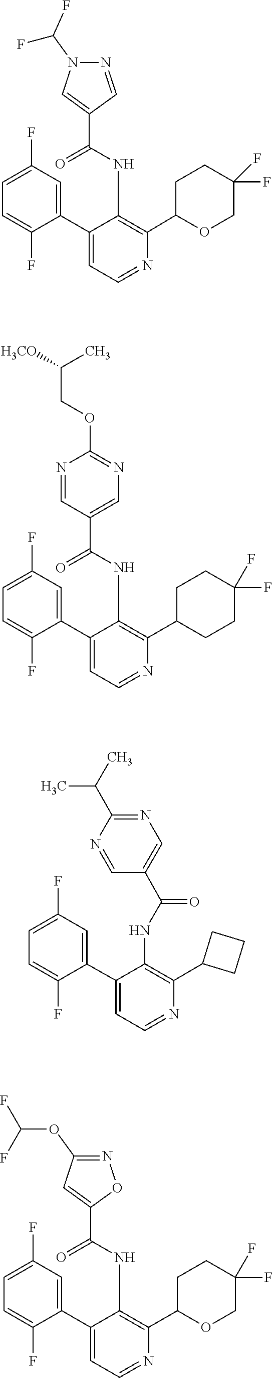 [SMILES: O=C(Nc1c(-c2cc(F)ccc2F)ccnc1C1CCC(F)(F)CO1)c1cnn(C(F)F)c1 ; CO[C@H](C)COc1ncc(C(=O)Nc2c(-c3cc(F)ccc3F)ccnc2C2CCC(F)(F)CC2)cn1 ; CC(C)c1ncc(C(=O)Nc2c(-c3cc(F)ccc3F)ccnc2C2CCC2)cn1 ; O=C(Nc1c(-c2cc(F)ccc2F)ccnc1C1CCC(F)(F)CO1)c1cc(OC(F)F)no1] 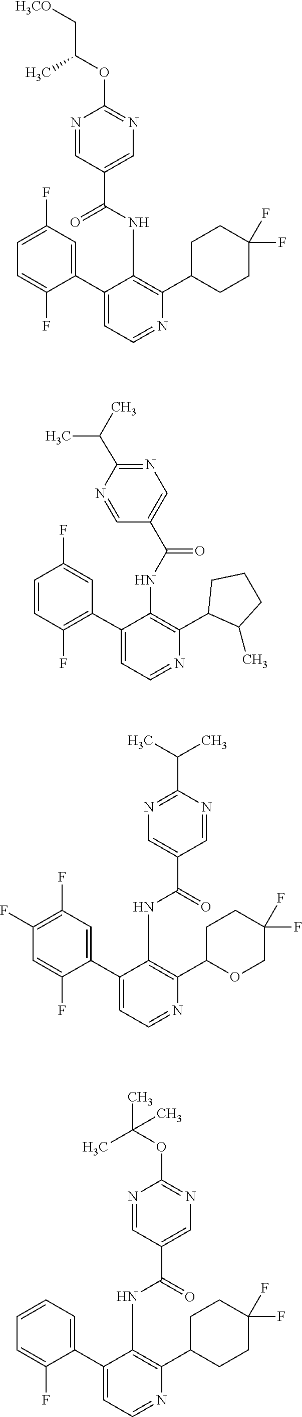 [SMILES: COC[C@@H](C)Oc1ncc(C(=O)Nc2c(-c3cc(F)ccc3F)ccnc2C2CCC(F)(F)CC2)cn1 ; CC(C)c1ncc(C(=O)Nc2c(-c3cc(F)ccc3F)ccnc2C2CCCC2C)cn1 ; CC(C)c1ncc(C(=O)Nc2c(-c3cc(F)c(F)cc3F)ccnc2C2CCC(F)(F)CO2)cn1 ; CC(C)(C)Oc1ncc(C(=O)Nc2c(-c3ccccc3F)ccnc2C2CCC(F)(F)CC2)cn1] 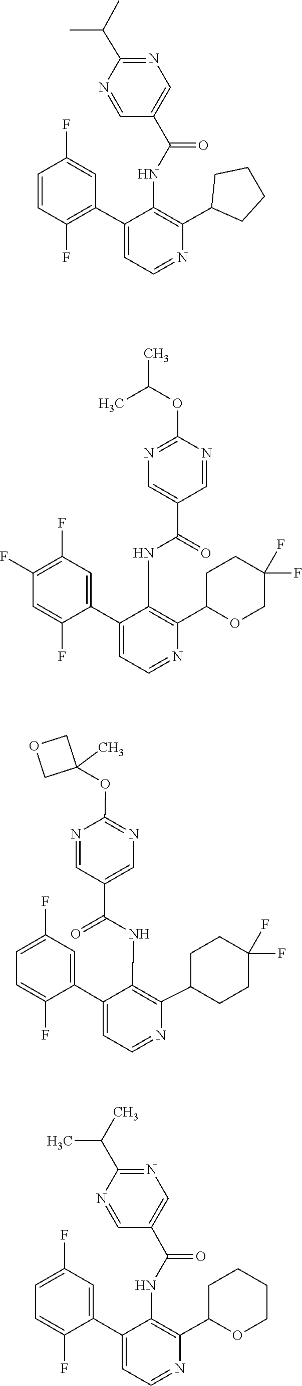 [SMILES: CC(C)c1ncc(C(=O)Nc2c(-c3cc(F)ccc3F)ccnc2C2CCCC2)cn1 ; CC(C)Oc1ncc(C(=O)Nc2c(-c3cc(F)c(F)cc3F)ccnc2C2CCC(F)(F)CO2)cn1 ; CC1(Oc2ncc(C(=O)Nc3c(-c4cc(F)ccc4F)ccnc3C3CCC(F)(F)CC3)cn2)COC1 ; CC(C)c1ncc(C(=O)Nc2c(-c3cc(F)ccc3F)ccnc2C2CCCCO2)cn1] 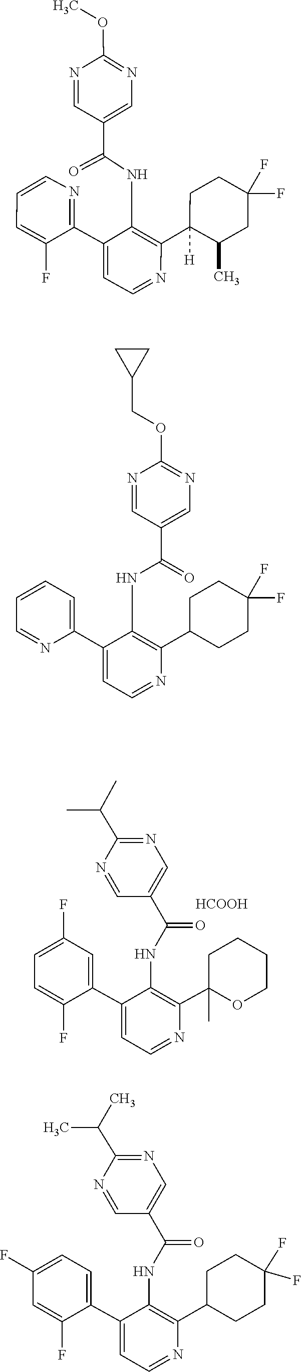 [SMILES: COc1ncc(C(=O)Nc2c(-c3ncccc3F)ccnc2[C@H]2CCC(F)(F)C[C@H]2C)cn1 ; O=C(Nc1c(-c2ccccn2)ccnc1C1CCC(F)(F)CC1)c1cnc(OCC2CC2)nc1 ; CC(C)c1ncc(C(=O)Nc2c(-c3cc(F)ccc3F)ccnc2C2(C)CCCCO2)cn1 ; "HCOOH" ; CC(C)c1ncc(C(=O)Nc2c(-c3ccc(F)cc3F)ccnc2C2CCC(F)(F)CC2)cn1] 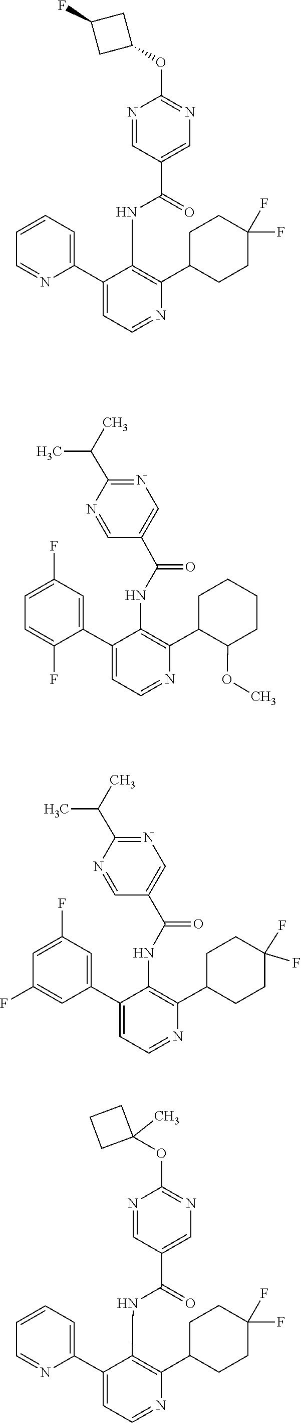 [SMILES: O=C(Nc1c(-c2ccccn2)ccnc1C1CCC(F)(F)CC1)c1cnc(O[C@H]2C[C@H](F)C2)nc1 ; COC1CCCCC1c1nccc(-c2cc(F)ccc2F)c1NC(=O)c1cnc(C(C)C)nc1 ; CC(C)c1ncc(C(=O)Nc2c(-c3cc(F)cc(F)c3)ccnc2C2CCC(F)(F)CC2)cn1 ; CC1(Oc2ncc(C(=O)Nc3c(-c4ccccn4)ccnc3C3CCC(F)(F)CC3)cn2)CCC1] 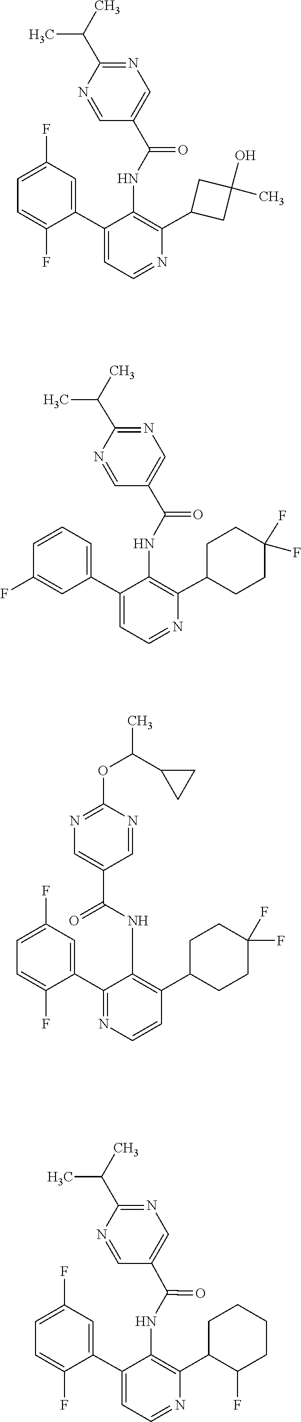 [SMILES: CC(C)c1ncc(C(=O)Nc2c(-c3cc(F)ccc3F)ccnc2C2CC(C)(O)C2)cn1 ; CC(C)c1ncc(C(=O)Nc2c(-c3cccc(F)c3)ccnc2C2CCC(F)(F)CC2)cn1 ; CC(Oc1ncc(C(=O)Nc2c(C3CCC(F)(F)CC3)ccnc2-c2cc(F)ccc2F)cn1)C1CC1 ; CC(C)c1ncc(C(=O)Nc2c(-c3cc(F)ccc3F)ccnc2C2CCCCC2F)cn1] 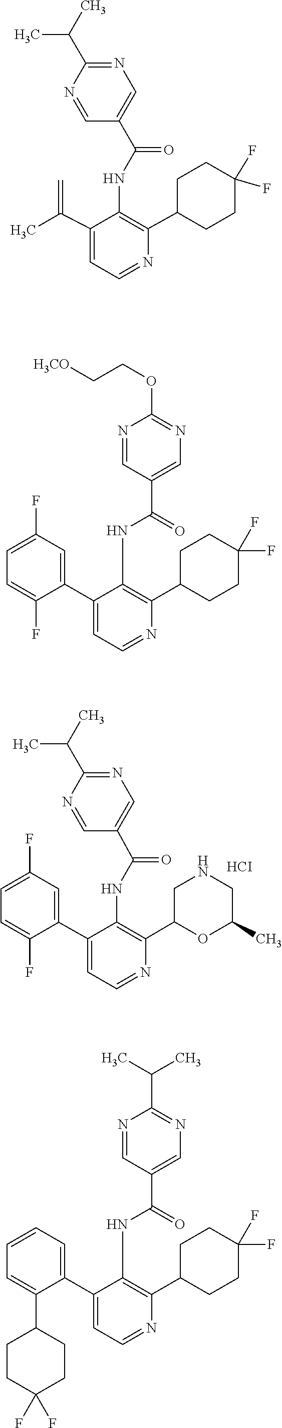 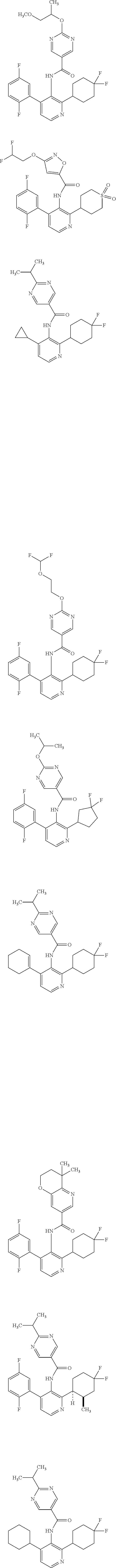 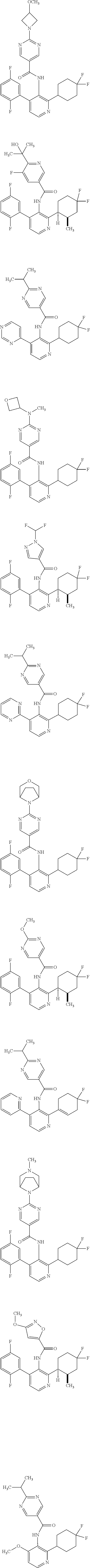 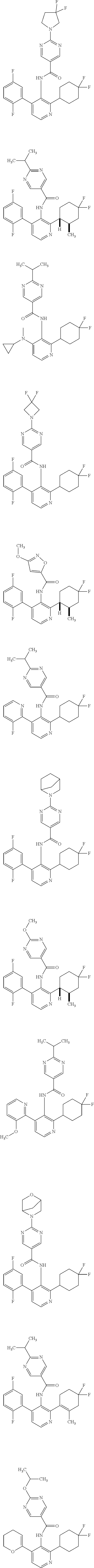 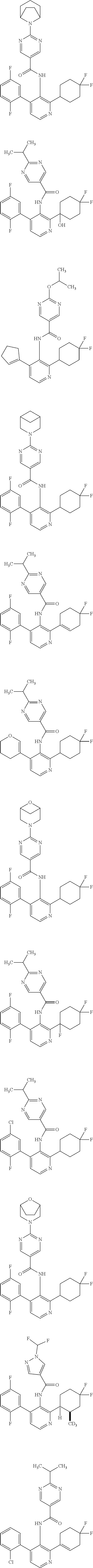 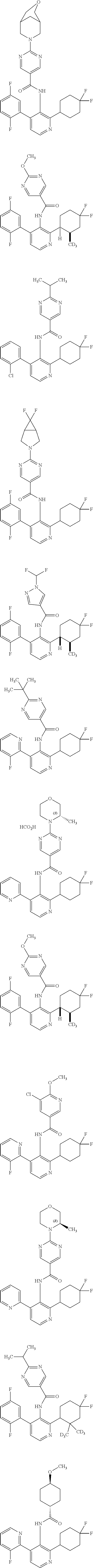 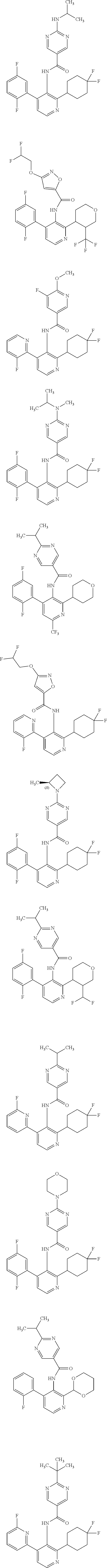 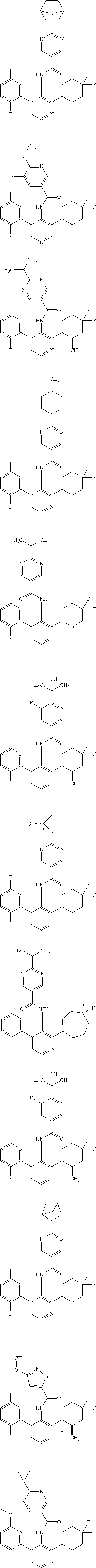 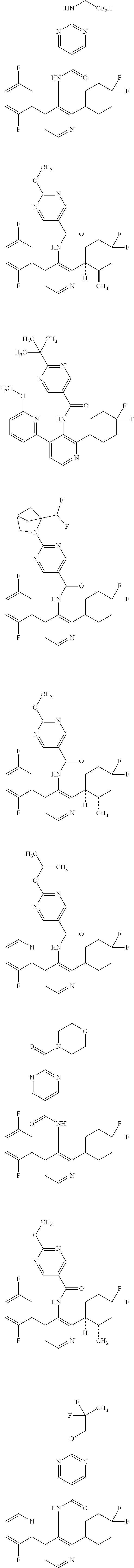 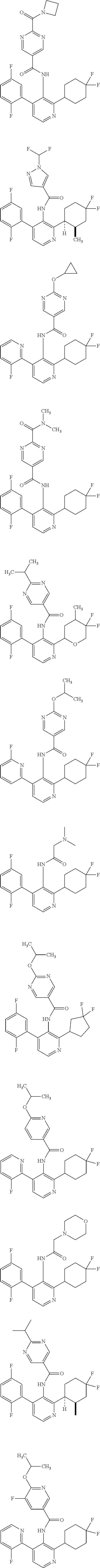 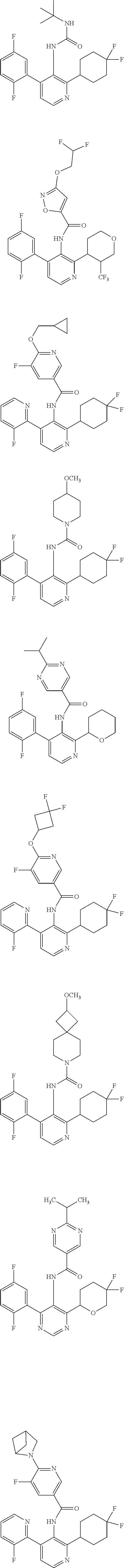 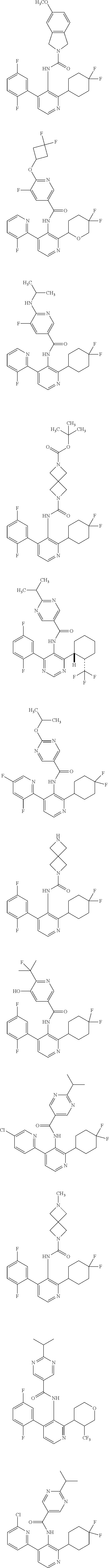 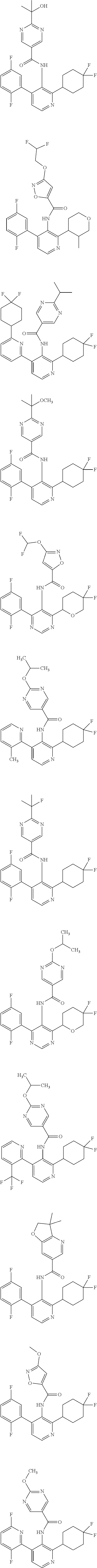 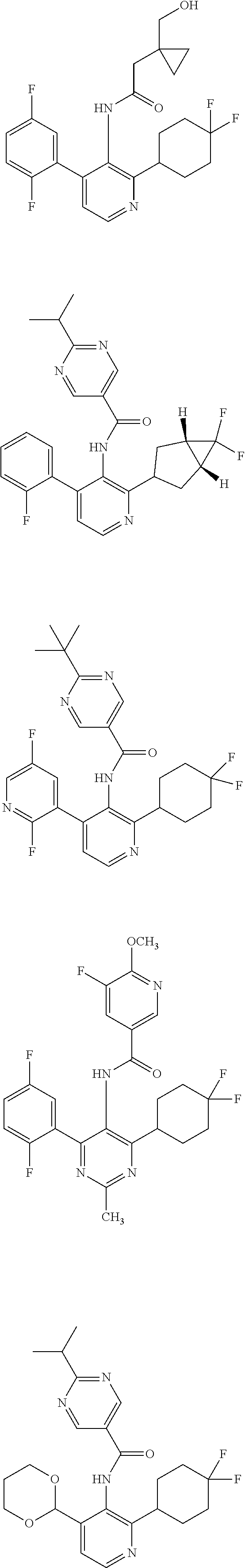 [SMILES: O=C(CC1(CO)CC1)Nc1c(-c2cc(F)ccc2F)ccnc1C1CCC(F)(F)CC1 ; CC(C)c1ncc(C(=O)Nc2c(-c3ccccc3F)ccnc2C2C[C@@H]3[C@H](C2)C3(F)F)cn1 ; CC(C)(C)c1ncc(C(=O)Nc2c(-c3cc(F)cnc3F)ccnc2C2CCC(F)(F)CC2)cn1 ; COc1ncc(C(=O)Nc2c(-c3cc(F)ccc3F)nc(C)nc2C2CCC(F)(F)CC2)cc1F ; CC(C)c1ncc(C(=O)Nc2c(C3OCCCO3)ccnc2C2CCC(F)(F)CC2)cn1]
as a stereoisomer, enantiomer, or tautomer thereof or a mixture thereof, or a pharmaceutically acceptable salt, solvate, isotopologue, or prodrug thereof.

72. A pharmaceutical composition comprising a pharmaceutically acceptable excipient and a compound of formula (I): 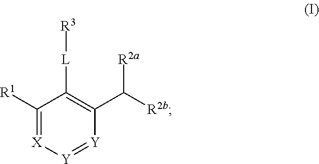
wherein:
X, Y, and Z are each independently N or CR^{1b}, provided that at least one and no more than two of X, Y, and Z are N;
L is a direct bond, —NR⁴C(═O)—, or —C(═O)NR⁴—;
R¹ is methoxy, —R⁵N(R⁶)₂, alkenyl, or has one of the following structures: 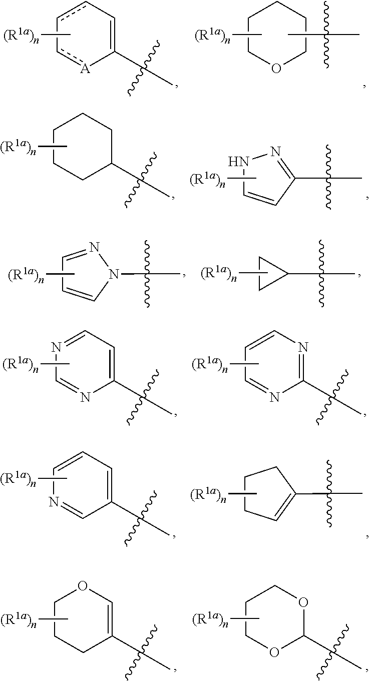
wherein:
each [symbol] is independently a single or double bond such that all valences are satisfied;
each R^{1a} is independently alkyl, halo, haloalkyl, —R⁵OR⁶, —R⁵N(R⁶)₂, —R⁵OC(═O)R⁶, optionally substituted cycloalkyl, or —R⁵C(═O)OR⁶;
A is O, N, or C;
each R^{1b} is independently hydrogen, halo, alkyl, or haloalkyl;
R^{2a} and R^{2b}, together with the carbon to which they are attached, form an optionally substituted cycloalkyl, an optionally substituted cycloalkenyl, an optionally substituted aryl, an optionally substituted heterocyclyl, or an optionally substituted heteroaryl;
or R^{2a} is hydrogen or alkyl and R^{2b} is an optionally substituted heterocyclyl or an optionally substituted cycloalkyl;
or R^{2a} and R^{2b} are both alkyl;
or R^{2a} is alkyl and R^{2b} is haloalkoxy;
R³ is alkyl, cyanoalkyl, —R⁵OR⁶, —R⁵N(R⁶)₂, an optionally substituted cycloalkyl, an optionally substituted aryl, an optionally substituted heterocyclyl, an optionally substituted heteroaryl, an optionally substituted cycloalkylalkyl, an optionally substituted heterocyclylalkyl;
R⁴ is hydrogen or alkyl;
each R⁵ is independently a direct bond or an optionally substituted alkylene chain;
each R⁶ is independently hydrogen, alkyl, haloalkyl, optionally substituted cycloalkyl, or optionally substituted arylalkyl;
or two R⁶'s, together with the nitrogen to which they are both attached, form an optionally substituted heterocyclyl; and
n is 0, 1, 2, 3, 4, or 5,
as a stereoisomer, enantiomer, or tautomer thereof or a mixture thereof, or a pharmaceutically acceptable salt, solvate, isotopologue, or prodrug thereof.

73. A method of treating a disease or condition in a mammal modulated by a voltage-gated sodium channel, wherein the method comprises administering to a mammal in need thereof a therapeutically effective amount of a compound of formula (I): 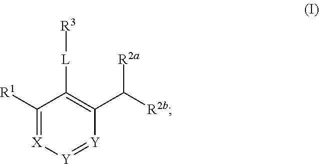
wherein:
X, Y, and Z are each independently N or CR^{1b}, provided that at least one and no more than two of X, Y, and Z are N;
L is a direct bond, —NR⁴C(═O)—, or —C(═O)NR⁴—;
R¹ is methoxy, —R⁵N(R⁶)₂, alkenyl, or has one of the following structures: 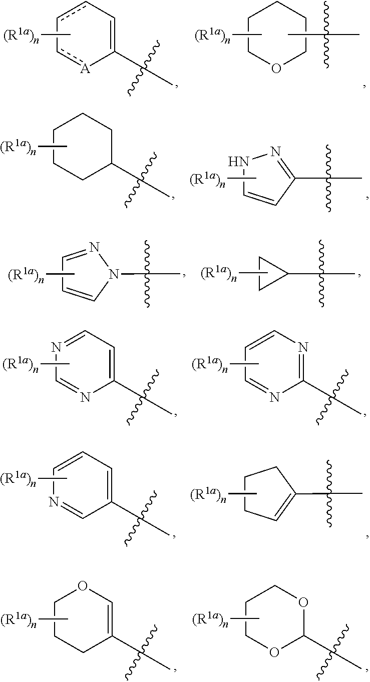
wherein:
each [symbol] is independently a single or double bond such that all valences are satisfied;
each R^{1a} is independently alkyl, halo, haloalkyl, —R⁵OR⁶, —R⁵N(R⁶)₂, —R⁵OC(═O)R⁶, optionally substituted cycloalkyl, or —R⁵C(═O)OR⁶;
A is O, N, or C;
each R^{1b} is independently hydrogen, halo, alkyl, or haloalkyl;
R^{2a} and R^{2b}, together with the carbon to which they are attached, form an optionally substituted cycloalkyl, an optionally substituted cycloalkenyl, an optionally substituted aryl, an optionally substituted heterocyclyl, or an optionally substituted heteroaryl;
or R^{2a} is hydrogen or alkyl and R^{2b} is an optionally substituted heterocyclyl or an optionally substituted cycloalkyl;
or R^{2a} and R^{2b} are both alkyl;
or R^{2a} is alkyl and R^{2b} is haloalkoxy;
R³ is alkyl, cyanoalkyl, —R⁵OR⁶, —R⁵N(R⁶)₂, an optionally substituted cycloalkyl, an optionally substituted aryl, an optionally substituted heterocyclyl, an optionally substituted heteroaryl, an optionally substituted cycloalkylalkyl, an optionally substituted heterocyclylalkyl;
R⁴ is hydrogen or alkyl;
each R⁵ is independently a direct bond or an optionally substituted alkylene chain;
each R⁶ is independently hydrogen, alkyl, haloalkyl, optionally substituted cycloalkyl, or optionally substituted arylalkyl;
or two R⁶'s, together with the nitrogen to which they are both attached, form an optionally substituted heterocyclyl; and
n is 0, 1, 2, 3, 4, or 5,
as a stereoisomer, enantiomer, or tautomer thereof or a mixture thereof, or a pharmaceutically acceptable salt, solvate, isotopologue, or prodrug thereof.
